(19) United States
(12) Patent Application Publication
(10) Pub. No.: US 2026/0325678 A1
(43) Pub. Date: Oct. 22, 2026

(54) Chimeric Antigen Receptors Targeting CD19

(71) Applicants: Orna Therapeutics, Inc., Watertown, MA (US); ReNAgade Therapeutics Management Inc., Watertown, MA (US)

(72) Inventors: Isin Dalkilic-Liddle, Watertown, MA (US); Robert Mabry, Watertown, MA (US); Thomas Lee, Watertown, MA (US); Karolina Anna Kosakowska, Watertown, MA (US); Muthusamy Jayaraman, Walpole, MA (US); Ganapathy Subramanian Sankaran, Watertown, MA (US)

(73) Assignees: Orna Therapeutics, Inc., Watertown, MA (US); ReNAgade Therapeutics Management Inc., Watertown, MA (US)

(21) Appl. No.: 19/288,568

(22) Filed: Aug. 1, 2025

Related U.S. Application Data
(60) Provisional application No. 63/714,186, filed on Oct. 31, 2024.
(60) Provisional application No. 63/744,463, filed on Jan. 13, 2025.
(60) Provisional application No. 63/793,971, filed on Apr. 24, 2025.
(60) Provisional application No. 63/802,341, filed on May 8, 2025.
(60) Provisional application No. 63/825,195, filed on Jun. 17, 2025.
(60) Provisional application No. 63/844,433, filed on Jul. 15, 2025.

(51) Int. Cl. A61K 48/00; A61K 31/7105; A61K 35/17; A61K 40/11; A61K 40/31; A61K 40/42; A61P 35/00; C07K 16/28; C07K 16/44; C12N 5/0783

(52) U.S. Cl. CPC A61K 48/005; A61K 31/7105; A61K 35/17; A61K 40/11; A61K 40/31; A61K 40/4211; A61K 48/0041; A61P 35/00; C07K 16/2803; C07K 16/44; C12N 5/0636; A61K 2239/13; A61K 2239/48; C07K 2317/31; C07K 2317/565; C07K 2317/622; C12N 2510/00

(57) ABSTRACT

Provided herein are RNA sequences encoding a chimeric antigen receptor (CAR) comprising a CD19 antigen binding molecule, along with related compositions and methods. In some embodiments, circular RNAs encoding the CD19 binding molecule or CD19 CAR, along with related compositions, methods, and precursors, are described herein. In some embodiments, these compositions comprise a transfer vehicle, e.g., comprising an ionizable lipid described herein.

### CROSS-REFERENCE TO RELATED APPLICATIONS

This application claims priority to U.S. Application No. 63/844,433, filed Jul. 15, 2025, U.S. Application No. 63/825,195, filed Jun. 17, 2025, U.S. Application No. 63/802,341, filed May 8, 2025, U.S. Application No. 63/793,971, filed Apr. 24, 2025, U.S. Application No. 63/744,463, filed Jan. 13, 2025, and U.S. Application No. 63/714,186, filed Oct. 31, 2024, the entire contents of which are incorporated by reference herein for all purposes.

### REFERENCE TO A SEQUENCE LISTING XML

The present application contains a Sequence Listing which has been submitted electronically in XML format. Said XML copy, created on Jul. 31, 2025, is named “01318-0017-00US_SL.xml” and is 684,539 bytes in size. The information in the electronic format of the sequence listing is incorporated herein by reference in its entirety.

Adoptive T-cell immunotherapy is a rapidly growing field, in particular, in cancer treatments. However, while CAR-T therapies have become an important tool in cancer treatments, they have toxic side effects and involve complex procedures. Treatment with CAR-T can lead to a large and rapid release of cytokines into the blood and can cause cytokine release syndrome (CRS) or CAR-T cell-related encephalopathy syndrome (CRES), also referred to as neurotoxicity associated with CAR-T. CRS is the most common and well-described toxicity associated with CAR-T therapy, occurring in over 90% of patients at any grade and is characterized by high fever, hypotension, hypoxia and/or multiple organ toxicity and can lead to death. Neurotoxicity is characterized by damage to nervous tissue that can cause tremors, encephalopathy, dizziness or seizures. Additionally, prior to infusion, the patients generally undergo lymphodepletion. Lymphodepletion is known to increase CAR-T cell expansion and enhance efficacy of infused CAR-T cells by, for example, altering the tumor phenotype and microenvironment. However, lymphodepletion agents often cause side effects to the patients. For example, lymphodepletion can cause neutropenia, anemia, thrombocytopenia, and immunosuppression, leading to a greater risk of infection, along with other toxicities. In addition to the toxicities associated with targeted CAR-T therapies, there are procedures, specialized equipment, and costs involved in producing the modified lymphocytes. CAR-T therapies require an assortment of protocols to isolate, genetically modify, and selectively expand the redirected cells before infusing them back into the patient.

In anti-CD19 CAR T cell therapy for refractory systemic lupus erythematosus, autologous T cells from five SLE patients were transduced with a lentiviral anti-CD19 CAR vector, expanded and reinfused into the patients after lymphodepletion with fludarabine and cyclophosphamide. CAR T cells expanded in vivo led to deep depletion of B cells, improvement of clinical symptoms and normalization of laboratory parameters including seroconversion of anti-double-stranded DNA antibodies. “Remission of SLE according to DORIS criteria was achieved in all five patients after 3 months and the median (range) Systemic Lupus Erythematosus Disease Activity Index score after 3 months was 0 (2).” See Mackensen et al., Anti-CD19 CAR T cell therapy for refractory systemic lupus erythematosus, Nature Medicine (2022); see also Nunez et al., Cytokine and reactivity profiles in SLE patients following anti-CD19 CART therapy, Molecular Therapy (2023).

Circular RNA (circRNA or oRNA®) is a stable form of RNA that provides an advantage compared to linear RNA in structure and function, especially in the case of molecules that are prone to folding in an inactive conformation (Wang and Ruffner, 1998). Circular RNA polynucleotides lack the free ends necessary for exonuclease-mediated degradation, causing them to be resistant to several mechanisms of RNA degradation and granting extended half-lives when compared to, e.g., linear mRNA comprising the same expression sequence. Circularization may allow for the stabilization of RNA polynucleotides that generally suffer from short half-lives and may improve the overall efficacy of exogenous mRNA in a variety of applications. Circular RNA can also be particularly interesting and useful for in vivo applications, especially in the research area of RNA-based control of gene expression and therapeutics.

Because circRNAs are more stable and can be expressed in tissue-specific manner, and because using circRNAs can avoid the lymphodepletiose n step of traditional therapies, circRNAs provide an attractive alternative to traditional CAR therapies and other therapies. Accordingly, provided herein are circular RNA constructs that comprise an internal ribosome entry site (IRES) and at least one expression sequence encoding a CD19 binding molecule. In certain embodiments, the binding molecule encodes a CAR that targets a CD19 molecule (e.g., a CD19 antigen molecule), for use in treating cancer. The circular RNA can be formulated with a transfer vehicle to facilitate and/or enhance the delivery and release of circRNA to one or more target cells. Accordingly, lipid nanoparticles (LNPs) or other transfer vehicles containing ionizable lipids may be used to deliver the circular RNA described herein, for example, to a patient in need of treatment.

### BRIEF DESCRIPTION OF THE DRAWINGS

FIGS. 1A-IF show a schematic of selected internal ribosome entry sites (IRES) selected for construct design of oRNA. FIG. 1A depicts a general sequence construct of a linear RNA polynucleotide precursor (10). The sequence as provided is illustrated in a 5′ to 3′ order of a 5′ enhanced intron element (20), a 5′ enhanced exon element (30), a core functional element (40), a 3′ enhanced exon element (50) and a 3′ enhanced intron element (60). FIG. 1B shows an exemplary linear RNA polynucleotide precursor (10) comprising in the following 5′ to 3′ order, a leading untranslated sequence (21), a 5′ affinity tag (22), a 5′ external spacer (26), a 3′ intron fragment (28), a 3′ exon fragment (32), a 5′ internal duplex region (34), a 5′ internal spacer (36), a TIE (42), a coding element (46), a stop region (48), a 3′ internal spacer (52), a 3′ internal duplex region (54), a 5′ exon fragment (56), a 5′ intron fragment (62), a 3′ external spacer (64), a 3′ affinity tag (68), and a terminal untranslated sequence (69). FIG. 1C illustrates exemplary locations for an accessory element (70) (e.g., a miRNA binding site) included in a linear RNA polynucleotide located within the core functional element (40), for example where 42 is the TIE (translation initiation element), 46 is the coding region, 47 is the noncoding region, and 48 is the stop region (stop codon or stop cassette). IRES candidates (i.e., synthetic and non-synthetic IRES) were ranked according to: (1) absorbance profile of the polysome species contained in sucrose gradients collected from cells translating circular RNAs having the IRES (depicted in FIG. 1D); and (2): half-life of circular RNAs having the IRES (illustrated in FIG. 1E). FIG. 1F depicts the combined relationship between the polysome absorbance profile and the half-life stability used to select the IRES candidates.

FIGS. 2A-2F depict anti-CD19 chimeric antigen receptor (CAR) expression (e.g., via mean fluorescent activity (gMFI)) and durability in primary human T cells post electroporation of circular RNA comprising an internal ribosome entry site (IRES) from Table 4 and encoding a CD19-CD28ζ CAR. The circular RNAs were generated from an IVT reaction of DNA Templates 1-30 from Table αα. The primary human T cells were treated with commercially available CD19 CAR biotin detection reagent containing streptavidin and R-phycoerythrin (PE) fluorophore (indicated as “sCD19 biotin-R-PE”). FIG. 2A depicts flow cytometry results at 24 hours (left) and 72 hours (right) for circular RNAs encoding CD19 CAR further comprising Binder X. FIG. 2B depicts flow cytometry results at 24 hours (left) and 72 hours (right) for circular RNAs encoding CD19 CAR further comprising Binder Y. FIG. 2C-2F shows expression at 24 hours (FIG. 2C), expression at 72 hours (FIG. 2D), total expression (FIG. 2E), and durability for CD19-CD28ζ CARs comprising Binder X (left) or Binder Y (right) (FIG. 2F).

FIGS. 3A-3B depict anti-CD19 chimeric antigen receptor (CAR) expression (e.g., via mean fluorescent activity (gMFI) and flow cytometry analysis) in primary human T cells (FIG. 3A) and in cynomolgus macaque T cells (FIG. 3B) 24 hours post transfection of circular RNA comprising an internal ribosome entry site (IRES) from Table 4 and encoding a CD19-CD28ζ CAR formulated in LNP (e.g., using Formulation A). The circular RNAs comprised Binder X or Binder Y. The primary human T cells were treated with commercially available CD19 CAR biotin detection reagent containing streptavidin and R-phycoerythrin (PE) fluorophore (indicated as “sCD19 biotin-R-PE”).

FIGS. 4A-4C depict anti-CD19 chimeric antigen receptor (CAR) expression (e.g., frequency or via mean fluorescent activity (gMFI)) or cytotoxic killing in primary human T cells post electroporation of circular RNA encoding a CD19-CD28ζ CAR. The circular RNAs were generated from an IVT reaction of DNA Templates 1, 31-35. The primary human T cells were treated with commercially available CD19 CAR biotin detection reagent containing streptavidin and R-phycoerythrin (PE) fluorophore (e.g., indicated as “SAV^{PE}”). “No stain” and “SAV^{PE}” were negative controls. FIGS. 4A-4B depict flow cytometry results at 24 hours post electroporation of circular RNAs encoding anti-CD19 CARs in human T cells. FIG. 4C illustrates calculated tumor cell killing within Nalm6 cells via percent cytotoxicity (indicated as “% killing”) at 24 hours post electroporation of circular RNAs encoding CD19-CD28ζ CAR with an effector-to-target (“E:T”) ratio of 1:10. % killing has been normalized to the mock control (indicated as “Mock”) comprising T cells not electroporated with circular RNA.

FIGS. 5A-5D depict anti-CD19 chimeric antigen receptor (CAR) expression (e.g., via mean fluorescent activity (gMFI)) and durability (e.g., ratio of gMFI at 72 hours compared to 24 hours) in primary cynomolgus T cells post transfection of circular RNA comprising an internal ribosome entry site (IRES) from Table 4 and encoding a CD19-CD28ζ CAR. Circular RNAs were formulated into LNPs (e.g., using Formulation A to form oRNA-LNPs). The circular RNAs comprised of either Binder B, Binder C, Binder D, or Binder E. FIG. 5A depicts flow cytometry analysis (e.g., via “% CAR”) for the circular RNAs encoding anti-CD19 CAR comprising one of five binders in cynomolgus T cells at 24 hours (FIG. 5A) and at 72 hours (FIG. 5B). FIG. 5C-5D depict gMFI expression for the circular RNAs encoding the anti-CD19 CARs at 24 hours (FIG. 5C) and at 72 hours (FIG. 5D) post transfection of the oRNA-LNPs.

FIGS. 6A-6B depict durability of anti-CD19 chimeric antigen receptor (CAR) expression (measured as area under the curve (“AUC”)) (FIG. 6A) and fold change of expression (measured using the geometric mean fluorescent intensity (gMFI) at 72 hours as compared to a 24-hour baseline)) (FIG. 6B) post transfection of oRNA-LNPs encoding CD19-CD28ζ CARs to human T cells. The circular RNAs comprised an internal ribosome entry site (IRES) from Table 4 and either a anti-CD19 CAR comprising Binder B, C, D or E.

FIGS. 7A-D depict frequency (e.g., at 24 hours (FIG. 7A) and 72 hours (FIG. 7B)) and mean fluorescent intensity (gMFI) (e.g., at 24 hours (FIG. 7C) and 72 hour (FIG. 7D) of human T cells post transfection with oRNA-LNP encoding CD19-CD28ζ chimeric antigen receptors (CARs) comprising different internal ribosome entry sites (IRES) from Table 4 and anti-CD19 CAR binders. The circular RNAs comprised of Binder B, C, D, or E. The human T cells were treated with commercially available soluble CD19 detection reagent containing R-phycoerythrin (PE) fluorophore (indicated as “sCD19-PE”).

FIGS. 8A-8H depict anti-CD19 chimeric antigen receptor (CAR) expression (e.g., frequency (%) or via mean fluorescent activity (gMFI)) in primary human T cells post transfection of circular RNA encoding a CD19-CD28ζ CAR formulated in LNP at varying doses. The circular RNAs were generated from an IVT reaction of DNA Templates 1, 36, 37, 42, 45, 46, 50, 51, 55, 64, 68, 73. “Mock” represents a “mock control” comprising T cells not transfected. FIG. 8A-8D depict flow cytometry results at 24 hours for circular RNAs encoding anti-CD19 CAR encoding anti-CD19 CAR binders. FIG. 8E-8H illustrate calculated tumor cell killing within Nalm6 cells via percent cytotoxicity (indicated as “% killing”) at 48 hours post co-culture. FIG. 8G-8H illustrates calculated tumor cell killing within humanized cynomolgus CD19 (K562.cyCD19) cells via percent cytotoxicity (indicated as “% killing”) at 48 hours post co-culture. % killing has been normalized to the mock control (indicated as “Mock”) comprising T cells not electroporated with circular RNA.

FIGS. 9A-9D depicts anti-CD19 chimeric antigen receptor (CAR) expression (e.g., frequency (%) (indicated as “% CAR positive”) or via mean fluorescent activity (gMFI)) in primary human T cells post transfection of circular RNA comprising an internal ribosome entry site (IRES) from Table 4 and encoding a CD19-CD28ζ CAR formulated in an LNP. The circular RNAs comprised of an anti-CD19 CAR comprising Binder B, C D, E, F, H, or I. “Mock” represents a “mock control” comprising T cells not transfected. FIGS. 9A-9B depict flow cytometry results at 24 hours for circular RNAs encoding an anti-CD19 CAR. FIGS. 9C-9D depict flow cytometry results at 72 hours.

FIGS. 10A-10D depict anti-CD19 chimeric antigen receptor (CAR) expression (e.g., frequency (%) (indicated as “% CAR positive”) or via mean fluorescent activity (gMFI)) in primary cynomolgus T cells post transfection of circular RNA comprising an internal ribosome entry site (IRES) from Table 4 and encoding a CD19-CD28ζ CAR formulated in an LNP. The circular RNAs comprised of an anti-CD19 CAR comprising Binder B, C D, E, F, H, or I. “Mock” represents a “mock control” comprising T cells not transfected. FIG. 10A-10B depict flow cytometry results at 24 hours for circular RNAs encoding an anti-CD19 CAR in two donor cells (e.g., Donor B (top) and Donor C (bottom)). FIGS. 10C-10D depict flow cytometry results at 72 hours in two donor cells (e.g., Donor B (top) and Donor C (bottom)).

FIGS. 11A-11B depict durability of anti-CD19 chimeric antigen receptor (CAR) expression (measured as area under the curve (“AUC”)) (FIG. 11A) and fold change of expression (measured using the geometric mean fluorescent intensity (gMFI) at 72 hours as compared to a 24-hour baseline)) (FIG. 11B) post transfection of oRNA-LNPs encoding CD19-CD28ζ CARs to human T cells. The circular RNAs comprised of an anti-CD19 CAR comprising Binder B, C D, E, F, H, or I and an internal ribosome entry site (IRES) from Table 4.

FIGS. 12A-12B depict anti-CD19 chimeric antigen receptor (CAR) expression (e.g. via mean fluorescence intensity (gMFI)) in primary cynomolgus T cells following LNP transfection. The circular RNAs were generated from an IVT reaction of DNA templates 1, 37, 64, 100, 137-186 and then formulated using Formulation A to create oRNA-LNPs. FIGS. 12A-12B depict flow cytometry results at 24 hours for circular RNAs encoding anti-CD19 CAR expression sequences comprising sequence alterations (e.g., codon optimizations, lysine to arginine mutations, or linker extension (e.g., at least one addition of a G4S linker) in two donor T cells (e.g., Donor D (illustrated in FIG. 12A) and Donor B (illustrated in FIG. 12B)). “Mock” control are T cells that were not transfected with circular RNAs.

FIGS. 13A-13C depict anti-CD19 chimeric antigen receptor (CAR) expression (e.g. via expression frequency of the entire population) in primary human T cells (FIG. 13A-13B) and in primary cynomolgus T cells (FIG. 13C) following oRNA-LNP transfection. Circular RNAs were generated from an IVT reaction of DNA templates 1, 37, 46, 100, 137-144, 147-153, 156-158, 171-173 and then formulated using Formulation A to create oRNA-LNPs. FIG. 13A-13C depict flow cytometry results at 24 hours (FIG. 13A and FIG. 13C) and 72 hours (FIG. 13B) for circular encoding anti-CD19 binders further comprising sequence alterations (e.g., codon optimizations, lysine to arginine mutations, or a linker extension (e.g., at least one addition of a G4S linker). “Mock” control are T cells that were not transfected with circular RNAs.

FIGS. 14A-14B illustrate anti-CD19 CAR expression (e.g. via mean fluorescence intensity (gMFI)) of primary human (FIG. 14A) and cynomolgus (FIG. 14B) T cells as compared to the calculated tumor cell killing (“% Killing”) elicited by the T cells against K562 expressing either human CD19 (indicated as “huCD19”) (FIG. 14A) or cynomolgus CD19 (indicated as “cyCD19”) (FIG. 14B) CAR. “Mock” control are T cells that were not transfected with circular RNAs. % Killing was collected at 72 hours after transfection and 48 hours post continuous co-culture.

FIGS. 15A-15B depict anti-CD19 chimeric antigen receptor (CAR) expression (e.g. via mean fluorescence intensity (gMFI)) in primary cynomolgus T cells following oRNA-LNP transfection. The circular RNAs were generated from an IVT reaction of DNA Templates 1, 37, 46, 64, 110, 139, 141, 152, 156, 171-173, 187-227 or 229 and formulated using Formulation A to create oRNA-LNPs. FIG. 15A-15B depict flow cytometry results at 24 hours for circular RNAs encoding anti-CD19 CAR Binders and comprising an internal ribosome entry site from Table 4. Circular RNAs encoded anti-CD19 CAR expression sequences further comprising sequence alterations (e.g., codon optimizations, lysine to arginine mutations, or linker extension (e.g., at least one addition of a G4S linker) in two donor T cells (e.g., Donor D (FIG. 15A) and Donor E (FIG. 15B)). “Mock” control are T cells that were not transfected with circular RNAs. FIGS. 15C-15D illustrate anti-CD19 CAR expression (e.g. via mean fluorescence intensity (gMFI)) of primary human (FIG. 15C) and cynomolgus (FIG. 15D) T cells as compared to the calculated tumor cell killing (“% Killing”) elicited by the T cells against K562 expressing either human CD19 (indicated as “huCD19”) (FIG. 15C) or cynomolgus CD19 (indicated as “cyCD19”) (FIG. 15D) CAR. “Mock” control are T cells that were not transfected with circular RNAs. % Killing was collected at 72 hours after transfection and 48 hours post continuous co-culture.

FIGS. 16A-16C depict anti-CD19 chimeric antigen receptor (CAR) expression (e.g. via durability of mean fluorescence intensity) in primary cynomolgus T cells following oRNA-LNP transfection. The circular RNAs were generated from an IVT reaction of DNA templates 1, 37, 46, 64, 100, 137—and formulated using Formulation A to create oRNA-LNPs. FIG. 16A-16B depict flow cytometry results as the area under the curve (AUC) of total expression from 24 hours and 72 hours post transfection of the oRNA-LNPs, wherein the circular RNAs encoding anti-CD19 CARs comprised either internal ribosome entry site (IRES) IRES C (FIG. 16A), IRES U (FIG. 16B), or IRES V (FIG. 16C). Circular RNAs encoded anti-CD19 CAR expression sequences further comprising sequence alterations (e.g., codon optimizations, lysine to arginine mutations, or linker extension (e.g., at least one addition of a G4S linker) in donor T cells (e.g., Donor D). “Mock” control are T cells that were not transfected with circular RNAs.

FIGS. 17A-17B depict anti-CD19 chimeric antigen receptor (CAR) expression (e.g. via expression frequency of the entire population) in primary human T cells following oRNA-LNP transfection. The circular RNAs were generated from an IVT reaction of DNA templates 1, 37, 46, 64, 100, 139-140, 153, 171-173, 188, 190, 201, 207-208, 217-218, or 225 and then formulated with Formulation A to create oRNA-LNPs. FIGS. 17A-17B depict flow cytometry results at 24 hours (FIG. 17A) and 72 hours (FIG. 17B). Circular RNAs encoded anti-CD19 CAR expression sequences further comprising sequence alterations (e.g., codon optimizations, lysine to arginine mutations, or linker extension (e.g., at least one addition of a G4S linker). “Mock” control are T cells that were not transfected with circular RNAs.

FIGS. 18A-18B illustrate anti-CD19 CAR expression (e.g. via mean fluorescence intensity (gMFI)) of primary human (FIG. 18A) and cynomolgus (FIG. 18B) T cells and the calculated tumor cell killing (“% Killing”) elicited by the T cells against K562 expressing either human CD19 (indicated as “huCD19”) (FIG. 18A) or cynomolgus CD19 (indicated as “cyCD19”) (FIG. 18B) protein at a 1:1 or 1:3 E:T, respectively. % Killing is collected at 72 hours after transfection and 48 hours of continuous co-culture. “Mock” control are T cells that were not transfected with circular RNAs.

FIG. 19 depicts calculated tumor cell killing within humanized CD19 (indicated as “K562.huCD19”) cells. Primary human T cells were transfected with circular RNA encoding a CD19-CD28ζ CAR formulated in LNP at varying doses (e.g., 0.5, 0.1, 0.5, 1. 5, 10 ng). The circular RNAs were generated from an IVT reaction of DNA Templates 1, 37, 42, 45, 46, 55, 64, 73, 153, 159, or 227. 24 hours after the transfection, effector T cells were co-cultured with K562.huCD19 cells. FIG. 19 illustrates the number of tumor cells remaining (indicated as “counts (GFP+CD3−)”) at 48 hours post co-culture with K562.huCD19 cells.

FIGS. 20A-20D depict anti-CD19 chimeric antigen receptor (CAR) expression (e.g., frequency (%) or via mean fluorescent activity (gMFI)) in primary cynomolgus T cells post transfection of circular RNA encoding a CD19-CD28ζ CAR formulated in LNP at varying doses. The circular RNAs were generated from an IVT reaction of DNA Templates 1, 37, 42, 45-46, 55, 64, 73, 153, 159. FIGS. 20A-20B depict flow cytometry results at 24 hours for circular RNAs encoding anti-CD19 CAR encoding an anti-CD19 CAR binder. FIG. 20C illustrates calculated tumor cell killing with K562.cyCD19 cells via tumor cells remaining (indicated as “count (GFP+CD3−)”) at 48 hours post co-culture. FIG. 20D depicts expression of CD19 CAR at 48 hours following co-culture in remaining primary cynomolgus T cells.

FIG. 21 shows B cells as % of baseline following administration of anti-CD19 oCAR (CD19 panCAR) formulated in LNP formulation F-B at 0.1 mg/kg and 0.3 mg/kg, with dexamethasone pre-treatment, to non-human primate as compared to negative control (a non-NHP cross-reactive anti-CD19 oCAR).

FIGS. 22A and 22B are bar graphs showing B cell depletion in the spleens of NHP subjects administered with CD19 panCAR constructs of the present disclosure, as reported in Example 15. FIG. 22A shows cells as a percentage of total CD45+ cells and FIG. 22B shows cells per gram of tissue.

FIGS. 23A and 23B are bar graphs showing B cell depletion in the mesenteric lymph nodes (mLN) of NHP subjects administered with CD19 panCAR constructs of the present disclosure, as reported in Example 15. FIG. 23A shows cells as a percentage of total CD45+ cells and FIG. 23B shows cells per gram of tissue.

FIGS. 24A and 24B are bar graphs showing B cell depletion in the pooled draining lymph nodes (dLN) of NHP subjects administered with CD19 panCAR constructs of the present disclosure, as reported in Example 15. FIG. 24A shows cells as a percentage of total CD45+ cells and FIG. 24B shows cells per gram of tissue.

FIGS. 25A and 25B are bar graphs showing B cell depletion in the bone marrow of NHP subjects administered with CD19 panCAR constructs of the present disclosure, as reported in Example 15. FIG. 25A shows cells as a percentage of total CD45+ cells and FIG. 25B shows cells normalized to B cells per 10⁶ Total Bone Marrow cells.

FIGS. 26A-26N are graphs showing B cell depletion in peripheral blood samples of NHP subjects administered with CD19 panCAR constructs of the present disclosure, as reported in Example 15. FIG. 26A shows B cells normalized as a percentage of baseline B cell values in peripheral blood up to Day 7. FIGS. 26B-26E are alternative representations of the data in FIG. 26A, broken down to show the results from the individual subjects. FIG. 26F shows B cells normalized as a percentage of baseline B cell values in peripheral blood up to Day 39. FIGS. 26G-26J are alternative representations of the data in FIG. 26F, broken down to show the results from the individual subjects. Dotted vertical lines in each of FIGS. 26B-26J indicate dosing timepoints. FIG. 26K shows CAR expression on T cells. FIG. 26L shows upregulation of an exemplary cytotoxicity marker. These data show that rapid cytotoxic effects occur after the first dose, and the majority of target cell killing occurs after the first dose. FIG. 26M shows peripheral B cell depletion over time after dosing. FIG. 26N shows reconstituted B cells (naive B cells and switched memory cells) over time after dosing.

FIGS. 27A-27C are bar graphs showing B Cell count in humanized mice dosed with LNPs F-S1 (VHH) or F-S2 (oCAR) as described in Example 16. FIGS. 27A, 27B, and 27C report B cell depletion in the peripheral blood, spleen and bone marrow (respectively) of the subject at Day 7, post 2×Q3D dosing.

FIGS. 27D-27E are bar graphs showing protein expression as determined from peripheral blood samples collected from humanized mice dosed with LNPs F-S1 (VHH) (FIG. 27D) or F-S2 (oCAR) (FIG. 27E), 24 hours post dose, as described in Example 16.

FIGS. 28A-28E are bar graphs showing peripheral blood B Cell counts in humanized mice dosed with LNPs F-S1 (VHH) or F-S2 (oCAR), 24 hours post dose, as described in Example 16.

FIGS. 29A-29E are bar graphs showing peripheral blood B Cell counts in humanized mice dosed with LNPs F-S1 (VHH) or F-S2 (oCAR), 72 hours post second dose on Day 4 (Day 7 total), as described in Example 16.

FIGS. 30A-30D are bar graphs showing splenic B Cell counts in humanized mice dosed with LNPs F-S1 (VHH) or F-S2 (oCAR), 72 hours post second dose on Day 4 (Day 7 total), as described in Example 16.

FIGS. 31A-31D are bar graphs showing bone marrow B Cell counts in humanized mice dosed with LNPs F-S1 (VHH) or F-S2 (oCAR), 72 hours post second dose on Day 4 (Day 7 total), as described in Example 16.

FIGS. 32A-32B are bar graphs showing protein expression as determined from peripheral blood samples collected from humanized mice dosed with LNP F-S2 (oCAR), at Day 5 (FIG. 32A) and Day 7 (FIG. 32B), as described in Example 17.

FIGS. 33A-33B are bar graphs showing peripheral blood B Cell counts in humanized mice dosed with LNPs F-S1 (VHH) or F-S2 (oCAR), 72 hours post second dose on Day 4 (Day 7 total), as described in Example 17.

FIGS. 34A-34B are bar graphs showing splenic B Cell counts in humanized mice dosed with LNPs F-S1 (VHH) or F-S2 (oCAR), 72 hours post second dose on Day 4 (Day 7 total), as described in Example 17.

FIGS. 35A-35B are bar graphs showing bone marrow B Cell counts in humanized mice dosed with LNPs F-S1 (VHH) or F-S2 (oCAR), 72 hours post second dose on Day 4 (Day 7 total), as described in Example 17.

FIGS. 36A-36E are CD19 IHC staining images of NHP spleens at 0.5× magnification after administration of PBS, or one of Formulations F-S1 or F-S2 at one of three doses (0.1, 0.5 and 1.0 mg/kg), 72 hours after the last (second) administered dose, as reported in Example 15. FIGS. 36A, 36B and 36C show the spleens of the subjects administered two doses of F-S2 at 0.1, 0.5 and 1.0 mg/kg, respectively. FIG. 36D shows the spleen of the subject administered PBS. FIG. 36E shows the spleen of the subject administered 1.0 mg/kg of Formulation F-S1. Spleens were collected as described in Example 15 and stained with CD19 IHC. Brown (darker) spots indicate positive CD19 staining while blue (lighter) spots are nuclei counterstains, providing contrast. A reduction in CD19 positive staining in the B-cell follicles, as well as the red pulp of the spleen, was observed in all three groups treated with F-S2, as compared to the PBS or F-S1 control groups.

FIG. 37A shows % CAR expression in blood for circular RNA (oRNA) CD19 CAR and linear mRNA CD19 CAR at 24 hours. FIG. 37B shows hCD20/CD19+ B cell counts in blood for oRNA CD19 CAR and mRNA CD19 CAR at 192 hours. FIG. 37C shows normalized hCD20/CD19+ B cell in blood for oRNA CD19 CAR and linear mRNA CD19 CAR at 192 hours. FIG. 37D shows % hCD20/CD19-All+ B cell frequency in blood for oRNA CD19 CAR and linear mRNA CD19 CAR at 192 hours. FIG. 37E shows normalized hCD20/CD19-All+ in blood for oRNA CD19 CAR and linear mRNA CD19 CAR at 192 hours. FIG. 37F shows hCD20/CD19-All+ frequency in spleen for oRNA CD19 CAR and linear mRNA CD19 CAR. FIG. 37G shows hCD20/CD19-All+ counts in spleen for oRNA CD19 CAR and linear mRNA CD19 CAR.

FIG. 38A shows % CAR expression for oRNA CD19 CAR and linear mRNA CD19 CAR over time. FIG. 38B shows Nalm6 GFP+ counts for oRNA CD19 CAR and linear mRNA CD19 CAR at 48 hours.

### DETAILED DESCRIPTION

Reference will now be made in detail to certain embodiments, examples of which are illustrated in the accompanying drawings. While the disclosure is described in conjunction with the illustrated embodiments, it will be understood that they are not intended to limit the disclosure to those embodiments. On the contrary, the disclosure is intended to cover all alternatives, modifications, and equivalents, which may be included within the disclosure as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### 1. Definitions

Unless stated otherwise, the following terms and phrases as used herein are intended to have the following meanings:

As used herein, linear nucleic acid molecules are said to have a “5′-terminus” (or “5′ end”) and a “3′-terminus” (or “3′ end”) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A “terminal nucleotide,” as used herein, is the nucleotide at the end position of the 3′- or 5′-terminus.

As used herein, the term “3′ intron segment” (or “3′ intron fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or 100% similarity to the 3′-proximal end of a natural intron (e.g., a group I or group II intron). In certain embodiments, the 3′ intron segment includes the 5′ nucleotide of the splice site dinucleotide. “3′ exon segment” (or “3′ exon fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or 100% similarity to the 5′-proximal end of an exon adjacent to a “3′ intron segment” as described herein. In certain embodiments, the 3′ exon segment includes the 3′ nucleotide of the splice site dinucleotide.

The term “5′ intron segment” (or “5′ intron fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or higher 100% similarity to the 5′-proximal end of a natural intron (e.g., a group I or group II intron). In certain embodiments, the 5′ intron segment includes the 3′ nucleotide of the splice site dinucleotide. “5′ exon segment” (or “5′ exon fragment”) refers to a sequence with at least 75%, at least 80%, at least 85%, at least 90%, at least 91%, at least 92%, at least 93%, at least 94%, at least 95%, at least 96%, at least 97%, at least 98%, at least 99%, or higher 100% similarity to the 3′-proximal end of an exon adjacent to a “5′ intron segment” as described herein. In certain embodiments, the 5′ exon segment includes the 5′ nucleotide of the splice site dinucleotide.

In some embodiments, the 3′ intron segment and the 3′ exon segment together form a first portion of an autocatalytic or self-splicing intron-exon sequence. In some embodiments, the 5′ intron segment and the 5′ exon segment together form the remainder (i.e., second portion) of the autocatalytic or self-splicing intron-exon sequence. In these embodiments, a linear nucleic acid molecule, e.g., RNA, comprising the 3′ intron segment and the 3′ exon segment at the 5′ end of the linear nucleic acid molecule and further the 5′ intron segment and the 5′ exon segment at the 3′ end the linear nucleic acid molecule, is capable of autocatalytically self-splicing and thereby capable of forming a circular nucleic acid molecule, e.g., circular RNA. In these embodiments, the 3′ intron segment and the 5′ intron segments are excised from the circular nucleic acid molecule, e.g., circular RNA, and the 3′ exon segment and the 5′ exon segment are retained in the circular nucleic acid molecule, e.g., circular RNA. Each retained post-splicing exon segment may be referred to as a self-splicing or self-spliced exon segment, e.g., a 3′ self-splicing or self-spliced exon segment and a 5′ self-splicing or self-spliced exon segment.

In some embodiments, the intron segment is a “Group I intron” and the corresponding exon segment may be referred to as a “Group I exon” or “Group 1 self-splicing exon” or “Group I self-spliced exon segment” or the like. In some embodiments, the intron segment is a “Group II intron” and the corresponding exon segment may be referred to as a “Group II exon” or “Group II self-splicing exon” or “Group II self-spliced exon segment” or the like.

In some embodiments, the retained, post-splicing, self-splicing 3′ or 5′ exon segment is a non-coding sequence in the circular nucleic acid molecule, e.g., circular RNA. In some embodiments, the circular nucleic acid molecule, e.g., circular RNA, further comprises a desired coding sequence, and the retained, post-splicing, self-splicing 3′ or 5′ exon segment is (e.g., designed) to be a portion of the desired expression sequence, contiguous with the desired coding sequence, and/or in frame with the desired coding sequence.

Within a circular nucleic acid molecule, e.g., derived from a linear nucleic acid precursor, and comprising a coding sequence, the 5′ to 3′ orientation of the coding sequence may be used to inform whether other sequences within the circular nucleic acid are 5′ and/or 3′, e.g., for example, 5′ is nearer to the 5′ of the coding sequence, and the 3′ end is downstream of the coding sequence. As used herein, within a circular nucleic acid molecule, e.g., derived from a linear nucleic acid precursor, reference to a “5′” or “3′” portion of the molecule may correspond to the orientation of the sequence within the linear nucleic acid precursor.

As used herein, “splice site” refers to the junction consisting of a dinucleotide between an exon and an intron in an unspliced RNA. As used herein, the term “splice site” refers to a dinucleotide that is partially or fully included in a group I or group II intron and/or exon and between which a phosphodiester bond is cleaved during RNA circularization. A “splice site dinucleotide” refers two nucleotides: a 5′ splice site nucleotide and the 3′ splice site nucleotide. A “5′ splice site” refers to the natural 5′ dinucleotide of the intron and/or exon e.g., group I or group II intron and/or exon, while a “3′ splice site” refers to the natural 3′ dinucleotide of the intron and/or exon. Exemplary splice site dinucleotides are shown in Table 1 below.

TABLE 1
Exemplary Splice
Site Dinucleotides

| 5' | 3' | 5' | 3' |
|---|---|---|---|
| nt of | nt of | nt of | nt of |
| a 3' | a 3' | a 5' | a 5' |
| splice | splice | splice | splice |
| site | site | site | site |
| di-nt | di-nt | di-nt | di-nt |
| A | A | A | A |
| A | A | A | C |
| A | A | A | G |
| A | A | A | U |
| A | A | C | A |
| A | A | C | C |
| A | A | C | G |
| A | A | C | U |
| A | A | G | A |
| A | A | G | C |
| A | A | G | G |
| A | A | G | U |
| A | A | U | A |
| A | A | U | C |
| A | A | U | G |
| A | A | U | U |
| A | C | A | A |
| A | C | A | C |
| A | C | A | G |
| A | C | A | U |
| A | C | C | A |
| A | C | C | C |
| A | C | C | G |
| A | C | C | U |
| A | C | G | A |
| A | C | G | C |
| A | C | G | G |
| A | C | G | U |
| A | C | U | A |
| A | C | U | C |
| A | C | U | G |
| A | C | U | U |
| A | G | A | A |
| A | G | A | C |
| A | G | A | G |
| A | G | A | U |
| A | G | C | A |
| A | G | C | C |
| A | G | C | G |
| A | G | C | U |
| A | G | G | A |
| A | G | G | C |
| A | G | G | G |
| A | G | G | U |
| A | G | U | A |
| A | G | U | C |
| A | G | U | G |
| A | G | U | U |
| A | U | A | A |
| A | U | A | C |
| A | U | A | G |
| A | U | A | U |
| A | U | C | A |
| A | U | C | C |
| A | U | C | G |
| A | U | C | U |
| A | U | G | A |
| A | U | G | C |
| A | U | G | G |
| A | U | G | U |
| A | U | U | A |
| A | U | U | C |
| A | U | U | G |
| A | U | U | U |
| C | A | A | A |
| C | A | A | C |
| C | A | A | G |
| C | A | A | U |
| C | A | C | A |
| C | A | C | C |
| C | A | C | G |
| C | A | C | U |
| C | A | G | A |
| C | A | G | C |
| C | A | G | G |
| C | A | G | U |
| C | A | U | A |
| C | A | U | C |
| C | A | U | G |
| C | A | U | U |
| C | C | A | A |
| C | C | A | C |
| C | C | A | G |
| C | C | A | U |
| C | C | C | A |
| C | C | C | C |
| C | C | C | G |
| C | C | C | U |
| C | C | G | A |
| C | C | G | C |
| C | C | G | G |
| C | C | G | U |
| C | C | U | A |
| C | C | U | C |
| C | C | U | G |
| C | C | U | U |
| C | G | A | A |
| C | G | A | C |
| C | G | A | G |
| C | G | A | U |
| C | G | C | A |
| C | G | C | C |
| C | G | C | G |
| C | G | C | U |
| C | G | G | A |
| C | G | G | C |
| C | G | G | G |
| C | G | G | U |
| C | G | U | A |
| C | G | U | C |
| C | G | U | G |
| C | G | U | U |
| C | U | A | A |
| C | U | A | C |
| C | U | A | G |
| C | U | A | U |
| C | U | C | A |
| C | U | C | C |
| C | U | C | G |
| C | U | C | U |
| C | U | G | A |
| C | U | G | C |
| C | U | G | G |
| C | U | G | U |
| C | U | U | A |
| C | U | U | C |
| C | U | U | G |
| C | U | U | U |
| G | A | A | A |
| G | A | A | C |
| G | A | A | G |
| G | A | A | U |
| G | A | C | A |
| G | A | C | C |
| G | A | C | G |
| G | A | C | U |
| G | A | G | A |
| G | A | G | C |
| G | A | G | G |
| G | A | G | U |
| G | A | U | A |
| G | A | U | C |
| G | A | U | G |
| G | A | U | U |
| G | C | A | A |
| G | C | A | C |
| G | C | A | G |
| G | C | A | U |
| G | C | C | A |
| G | C | C | C |
| G | C | C | G |
| G | C | C | U |
| G | C | G | A |
| G | C | G | C |
| G | C | G | G |
| G | C | G | U |
| G | C | U | A |
| G | C | U | C |
| G | C | U | G |
| G | C | U | U |
| G | G | A | A |
| G | G | A | C |
| G | G | A | G |
| G | G | A | U |
| G | G | C | A |
| G | G | C | C |
| G | G | C | G |
| G | G | C | U |
| G | G | G | A |
| G | G | G | C |
| G | G | G | G |
| G | G | G | U |
| G | G | U | A |
| G | G | U | C |
| G | G | U | G |
| G | G | U | U |
| G | U | A | A |
| G | U | A | C |
| G | U | A | G |
| G | U | A | U |
| G | U | C | A |
| G | U | C | C |
| G | U | C | G |
| G | U | C | U |
| G | U | G | A |
| G | U | G | C |
| G | U | G | G |
| G | U | G | U |
| G | U | U | A |
| G | U | U | C |
| G | U | U | G |
| G | U | U | U |
| U | A | A | A |
| U | A | A | C |
| U | A | A | G |
| U | A | A | U |
| U | A | C | A |
| U | A | C | C |
| U | A | C | G |
| U | A | C | U |
| U | A | G | A |
| U | A | G | C |
| U | A | G | G |
| U | A | G | U |
| U | A | U | A |
| U | A | U | C |
| U | A | U | G |
| U | A | U | U |
| U | C | A | A |
| U | C | A | C |
| U | C | A | G |
| U | C | A | U |
| U | C | C | A |
| U | C | C | C |
| U | C | C | G |
| U | C | C | U |
| U | C | G | A |
| U | C | G | C |
| U | C | G | G |
| U | C | G | U |
| U | C | U | A |
| U | C | U | C |
| U | C | U | G |
| U | C | U | U |
| U | G | A | A |
| U | G | A | C |
| U | G | A | G |
| U | G | A | U |
| U | G | C | A |
| U | G | C | C |
| U | G | C | G |
| U | G | C | U |
| U | G | G | A |
| U | G | G | C |
| U | G | G | G |
| U | G | G | U |
| U | G | U | A |
| U | G | U | C |
| U | G | U | G |
| U | G | U | U |
| U | U | A | A |
| U | U | A | C |
| U | U | A | G |
| U | U | A | U |
| U | U | C | A |
| U | U | C | C |
| U | U | C | G |
| U | U | C | U |
| U | U | G | A |
| U | U | G | C |
| U | U | G | G |
| U | U | G | U |
| U | U | U | A |
| U | U | U | C |
| U | U | U | G |
| U | U | U | U |

As used herein, the term “permutation site” refers to a site in an intron and/or exon (e.g., a group I or II intron and/or exon) where a cut is made prior to permutation of the intron/or exon. For example, such a cut generates an intron sequence comprising a 3′ intron segment and a sequence comprising a 5′ intron segment (e.g., group I or group II intron fragments) that are permuted to be on either side of a stretch of precursor RNA to be circularized. The permuted intron segments are thereby called “3′ permuted intron segments” or “3′ permuted elements” and “5′ permuted intron segments” or “5′ permuted elements” in the context of said precursor RNA. As used herein, “permuted intron segment” and “permuted intron element” are used interchangeably. In some embodiments, the permutation site consists of a dinucleotide.

As used herein, the singular forms “a,” “an,” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide.

A used herein, the terms “about,” or “approximately” are understood as within a range of normal tolerance in the art, for example within 2 standard deviations of the mean. “About” can be understood as within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value. Unless otherwise clear from the context, all numerical values provided herein are modified by the term “about.”

As used herein, “accessory element” or “accessory sequences” refer to internal spacer(s), external spacer(s), and/or homology arm(s). As used herein, a “combined accessory element” or “combined accessory sequences” comprises the accessory element and further comprises an intron and/or exon segment. In some embodiments, the accessory element increases circularization efficiency and/or translation efficiency in a circular RNA as compared to a control circular RNA without the accessory sequences.

As used herein, an “affinity sequence” or “affinity tag” is a region of a polynucleotide sequence ranging from one (1) nucleotide to hundreds or thousands of nucleotides containing a repeated set of nucleotides for the purposes of aiding purification of a polynucleotide sequence. For example, an affinity sequence may comprise, but is not limited to, a polyA or polyAC sequence. In some embodiments, affinity tags are used in purification methods, referred to herein as “affinity-purification,” in which selective binding of a binding agent to molecules comprising an affinity tag facilitates separation from molecules that do not comprise an affinity tag. In some embodiments, an affinity-purification method is a “negative selection” purification method, in which unwanted species, such as linear RNA, are selectively bound and removed and wanted species, such as circular RNA, are eluted and separated from unwanted species.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

An “antigen binding molecule,” “antigen binding portion,” or “antibody fragment” refers to any molecule that specifically binds to a desired antigen. In some embodiments, an antigen binding molecule comprises the antigen binding parts (e.g., CDRs) of an antibody or antibody-like molecule. An antigen binding molecule may include the antigenic complementarity determining regions (CDRs). Examples of antibody fragments include, but are not limited to, Fab, Fab′, F(ab′)2, Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In further embodiments, the antigen binding molecule is an antibody fragment, including one or more of the complementarity determining regions (CDRs) thereof, that specifically binds to the antigen. In further embodiments, the antigen binding molecule is a single chain variable fragment (scFv). In some embodiments, the antigen binding molecule comprises or consists of avimers.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain may comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions may be further subdivided into regions of hypervariability, termed complementarity determining regions (CDRs), interspersed with regions that are more conserved, termed framework regions (FR). CDRs may be described by numbering known in the art, for example, Kabat numbering, Chothia numbering, AbM numbering, or contact numbering. Each VH and VL may comprise three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component of the classical complement system. Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies (sdAb) (including, e.g., heavy chain-only antibodies (HcAbs), variable domain of new antigen receptor (VNAR), variable heavy domain of heavy chain (VHH)) or nanobodies), monovalent antibodies, single chain antibodies or single-chain variable fragments (scFv), camelid antibodies, affibodies, Fab fragments, F(ab′)2 fragments, disulfide-linked variable fragments (sdFv), anti-idiotypic (anti-id) antibodies (including, e.g., anti-anti-Id antibodies), minibodies, domain antibodies, synthetic antibodies (sometimes referred to herein as “antibody mimetics”), divalent single chain antibodies or diabodies and antigen-binding fragments of any of the above. In some embodiments, antibodies described herein refer to polyclonal antibody populations.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some embodiments, the variable region is a human variable region. In some embodiments, the variable region comprises rodent or murine CDRs and human framework regions (FRs). In particular embodiments, the variable region is a primate (e.g., non-human primate) variable region. In some embodiments, the variable region comprises rodent or murine CDRs and primate (e.g., non-human primate) framework regions (FRs). The terms “VL” and “VL domain” are used interchangeably to refer to the light chain variable region of an antibody or an antigen-binding molecule thereof. The terms “VH” and “VH domain” are used interchangeably to refer to the heavy chain variable region of an antibody or an antigen-binding molecule thereof.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which may exhibit various effector functions, such as interaction with the Fc receptor. The constant region of an immunoglobulin molecule generally has a more conserved amino acid sequence relative to an immunoglobulin variable domain.

As used herein, “aptamer” refers in general to either an oligonucleotide of a single defined sequence or a mixture of said nucleotides, wherein the mixture retains the properties of binding specifically to the target molecule (e.g., eukaryotic initiation factor, 40S ribosome, polyC binding protein, polyA binding protein, polypyrimidine tract-binding protein, argonaute protein family, Heterogeneous nuclear ribonucleoprotein K and La and related RNA-binding protein). Thus, as used herein “aptamer” denotes both singular and plural sequences of nucleotides, as defined hereinabove. The term “aptamer” is meant to refer to a single- or double-stranded nucleic acid which is capable of binding to a protein or other molecule. In general, aptamers preferably comprise about 10 to about 100 nucleotides, preferably about 15 to about 40 nucleotides, more preferably about 20 to about 40 nucleotides, in that oligonucleotides of a length that falls within these ranges are readily prepared by conventional techniques. Optionally, aptamers can further comprise a minimum of approximately 6 nucleotides, preferably 10, and more preferably 14 or 15 nucleotides, that are necessary to effect specific binding. In some embodiments, the circRNA described herein comprises and/or functions as an aptamer.

As used herein, “autoimmunity” is defined as persistent and progressive immune reactions to non-infectious self-antigens, as distinct from infectious non self-antigens from bacterial, viral, fungal, or parasitic organisms which invade and persist within mammals and humans. Autoimmune conditions include scleroderma, Grave's disease, Crohn's disease, Sjorgen's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, and thyroiditis, as well as in the generalized autoimmune diseases typified by human Lupus.

“Autoantigen” or “self-antigen” as used herein refers to an antigen or epitope which is native to the mammal and which is immunogenic in said mammal.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y may generally be represented by the dissociation constant (K_{D} or K_{d}). Affinity may be measured and/or expressed in a number of ways known in the art, including, but not limited to, equilibrium dissociation constant (K_{D}), and equilibrium association constant (K_{A} or Kₐ). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} may be determined by techniques known to one of ordinary skill in the art, such as BIACORE® or KinExA.

As used herein, the term “specifically binds,” refers to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a K_{A} that is at least 2 logs, 2.5 logs, 3 logs, 4 logs or greater than the K_{A} when the molecules bind to another antigen.

As used herein, “bicistronic RNA” refers to a polynucleotide that includes two expression sequences coding for two distinct proteins. These expression sequences can be separated by a nucleotide sequence encoding a cleavable peptide such as a protease cleavage site. They can also be separated by a ribosomal skipping element.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, multiple myeloma (MM), Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, neoplasm of the central nervous system (CNS), primary CNS lymphoma, tumor angiogenesis, spinal axis tumor, brain stem glioma, pituitary adenoma, epidermoid cancer, squamous cell cancer, T cell lymphoma, environmentally induced cancers including those induced by asbestos, other B cell malignancies, and combinations of said cancers. In some embodiments, the methods disclosed herein may be used to reduce the tumor size of a tumor derived from, for example, sarcomas and carcinomas, fibrosarcoma, myxosarcoma, liposarcoma, chondrosarcoma, osteogenic sarcoma, Kaposi's sarcoma, sarcoma of soft tissue, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinomna, lung cancer, colorectal cancer, squamous cell carcinoma, basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, carcinoma of the fallopian tubes, carcinoma of the endometrium, carcinoma of the cervix, carcinoma of the vagina, carcinoma of the vulva, carcinoma of the renal pelvis, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the terms “circRNA,” “circular polyribonucleotide,” “circular RNA,” “circularized RNA,” “circular RNA polynucleotide” and “oRNA” are used interchangeably and refer to a single-stranded polyribonucleotide wherein the 3′ and 5′ ends that are normally present in a linear RNA polynucleotide have been joined together, e.g., by covalent bonds. As used herein, such terms also include preparations comprising circRNAs.

As used herein, the term “circularization efficiency” refers to a measurement of the rate of formation of amount of resultant circular polyribonucleotide as compared to its linear starting material.

The expression sequences in the polynucleotide construct may be separated by a “cleavage site” sequence which enables polypeptides encoded by the expression sequences, once translated, to be expressed separately by the cell, e.g., eukaryotic cell. A “self-cleaving peptide” refers to a peptide which is translated without a peptide bond between two adjacent amino acids, or functions such that when the polypeptide comprising the proteins and the self-cleaving peptide is produced, it is immediately cleaved or separated into distinct and discrete first and second polypeptides without the need for any external cleavage activity.

As used herein, “co-administering” refers to administering a therapeutic agent provided herein in conjunction with one or more additional therapeutic agents sufficiently close in time such that the therapeutic agent provided herein can enhance the effect of the one or more additional therapeutic agents, or vice versa.

As used herein, “coding element,” “coding sequence,” “coding nucleic acid,” or “coding region” is region located within the expression sequence and encodings for one or more proteins or polypeptides (e.g., therapeutic protein).

As used herein, a “noncoding element,” “noncoding sequence,” “non-coding nucleic acid,” or “noncoding nucleic acid” is a region located within the expression sequence. This sequence by itself does not encode for a protein or polypeptide, but may have other regulatory functions, including but not limited, allow the overall polynucleotide to act as a biomarker or adjuvant to a specific cell.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-IBB ligand, agonist or antibody that binds Toll-like receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) LI. A co-stimulatory ligand includes, without limitation, an antibody that specifically binds with a co-stimulatory molecule present on a T cell, such as, but not limited to, 4-1BB/CD137, B7-H3, CD2, CD27, CD28, CD30, CD40, CD7, CD86, CD152, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD152, CD154, CD16, CD160 (BY55), CD 18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-la, CDl-lb, CDl-lc, CDl-ld, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, IT GAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD1 la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

As used herein, an antigen binding molecule, an antibody, or an antigen binding molecule thereof “cross-competes” with a reference antibody or an antigen binding molecule thereof if the interaction between an antigen and the first binding molecule, an antibody, or an antigen binding molecule thereof blocks, limits, inhibits, or otherwise reduces the ability of the reference binding molecule, reference antibody, or an antigen binding molecule thereof to interact with the antigen. Cross competition may be complete, e.g., binding of the binding molecule to the antigen completely blocks the ability of the reference binding molecule to bind the antigen, or it may be partial, e.g., binding of the binding molecule to the antigen reduces the ability of the reference binding molecule to bind the antigen. In some embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays may be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

A “cytokine,” as used herein, refers to a non-antibody protein that is released by one cell in response to contact with a specific antigen, wherein the cytokine interacts with a second cell to mediate a response in the second cell. A cytokine may be endogenously expressed by a cell or administered to a subject. Cytokines may be released by immune cells, including macrophages, B cells, T cells, neutrophils, dendritic cells, eosinophils and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-la, IL-lb, IL-6, IL-13, IL-17a, IL-23, IL-27, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), TGF-beta, IL-35, and perforin. Examples of acute phase-proteins include, but are not limited to, C-reactive protein (CRP) and serum amyloid A (SAA).

The terms “deoxyribonucleic acid” and “DNA” as used herein mean a polymer composed of deoxyribonucleotides. The terms “ribonucleic acid” and “RNA” as used herein mean a polymer composed of ribonucleotides.

As used herein, the term “DNA template” refers to a DNA sequence capable of transcribing a linear RNA polynucleotide. For example, but not intending to be limiting, a DNA template may include a DNA vector, PCR product or plasmid.

As used herein, the terms “duplexed,” “double-stranded,” and “hybridized” are used interchangeably and refer to double-stranded nucleic acids formed by hybridization of two single strands of nucleic acids containing complementary sequences. Sequences of the two single-stranded nucleic acids can be fully complementary or partially complementary. In some embodiments, a nucleic acid provided herein may be fully double-stranded or partially double-stranded. In most cases, genomic DNA is double-stranded.

As used herein, two “duplex sequences,” “duplex forming sequences,” “duplex region,” “duplex forming regions,” “homology arms,” or “homology regions,” complement, or are complementary, fully or partially, to one another when the two regions share a sufficient level of sequence identity to one another's reverse complement to act as substrates for a hybridization reaction. In some embodiments, two duplex forming sequences are thermodynamically favored to cross-pair in a sequence specific interaction. As used herein, polynucleotide sequences have “homology” when they are either identical or share sequence identity to a reverse complement or “complementary” sequence. The percent sequence identity between a homology region and a counterpart homology region's reverse complement can be any percent of sequence identity that allows for hybridization to occur. In some embodiments, an internal duplex forming region of a polynucleotide disclosed herein is capable of forming a duplex with another internal duplex forming region and does not form a duplex with an external duplex forming region.

As used herein, the term “encode” refers broadly to any process whereby the information in a polymeric macromolecule is used to direct the production of a second molecule that is different from the first. The second molecule may have a chemical structure that is different from the chemical nature of the first molecule. For example, a DNA template (e.g., a DNA vector) may encode a RNA polynucleotide; a precursor RNA polynucleotide (e.g., a linear precursor RNA polynucleotide) may encode a mature RNA polynucleotide (e.g., a circular RNA polynucleotide).

As used herein, “endogenous” means a substance that is native to, i.e., naturally originated from, a biological system (e.g., an organism, a tissue, or a cell). For example, in some embodiments, a “endogenous polynucleotide” is normally expressed in a cell or tissue. In some embodiments, a polynucleotide is still considered endogenous if the control sequences, such as a promoter or enhancer sequences which activate transcription or translation, have been altered through recombinant techniques.

As used herein, the term “heterologous” means from any source other than naturally occurring sequences.

As used herein, an “endonuclease site” refers to a stretch of nucleotides within a polynucleotide that is capable of being recognized and cleaved by an endonuclease protein.

An “eukaryotic initiation factor” or “eIF” refers to a protein or protein complex used in assembling an initiator tRNA, 40S and 60S ribosomal subunits required for initiating eukaryotic translation.

As used herein, an “epitope” is a term in the art and refers to a localized region of an antigen to which an antibody may specifically bind. An epitope may be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some embodiments, the epitope to which an antibody binds may be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen NE (1997) Structure 5: 1269-1274; McPherson A (1976) J Biol Chem 251: 6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. Patent Publication No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323).

As used herein, the term “expression sequence” refers to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon.”

As used herein, a “fusion protein” is a protein with at least two domains that are encoded by separate genes that have been joined to transcribe for a single peptide.

As used herein, the term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

As used herein, an “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

As used herein, the term “immunogenic” or “immunostimulatory” refers to a potential to induce an immune response to a substance. An immune response may be induced when an immune system of an organism or a certain type of immune cells is exposed to an immunogenic substance. The term “non-immunogenic” refers to a lack of or absence of an immune response above a detectable threshold to a substance. No immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cells is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

As used herein, an “internal ribosome entry site” or “IRES” refers to an RNA sequence or structural element capable of initiating translation of a polypeptide in the absence of a typical RNA cap structure. An IRES sequence may be naturally occurring or synthetic, e.g., derived from a naturally occurring virus IRES. In some embodiments, the IRES sequence ranges in size from 10 to 1000 nucleotides. In some embodiments, the IRES sequence if greater than 1000 nucleotides in size. In some embodiments, the IRES sequence ranges in size from 100 to 200 nucleotides, 201 to 300 nucleotides, 301 to 400 nucleotides, 401 to 500 nucleotides, 501 to 600 nucleotides, 601 to 700 nucleotides, 701 to 800 nucleotides, 801 to 900 nucleotides, 901 to 1000 nucleotides.

As used herein, an “intervening region” refers to the portion of an RNA sequence that comprises one or more noncoding or one or more coding elements, or combinations thereof (e.g., translation initiation element, coding element, and/or stop codon) between splice sites. In some embodiments, the intervening regions are between the 5′ combined accessory element and the 3′ combined accessory element or between the 3′ intron fragment and the 5′ intron fragment in a precursor RNA polynucleotide. In some embodiments, the intervening region is between the monotron element and terminal element in other precursor RNA polynucleotides.

As used herein, “isolated” or “purified” generally refers to isolation of a substance (for example, in some embodiments, a compound, a polynucleotide, a protein, a polypeptide, a polynucleotide composition, or a polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50, 60, 70, 75, 80, 85, 90, 95, 96, 97, 98, or 99% of the sample. In additional embodiments, a substantially purified component comprises about, 80%-85%, or 90%-95%, 95-99%, 96-99%, 97-99%, or 95-100% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is more than as it is found naturally.

As used herein, a “leading untranslated sequence” is a region of polynucleotide sequences ranging from 1 nucleotide to hundreds of nucleotides located at the upmost 5′ end of a polynucleotide sequence. The sequences can be defined or can be random. A leading untranslated sequence is non-coding.

As used herein, a “terminal untranslated sequence” is a region of polynucleotide sequences ranging from 1 nucleotide to hundreds of nucleotides located at the downmost 3′ end of a polynucleotide sequence. The sequences can be defined or can be random. A terminal untranslated sequence is non-coding.

As used herein, the terms “terminal sequence” or “terminal element” are used interchangeably to refer to an RNA sequence capable of complexing with a monotron sequence or monotron element. The terminal sequence comprises a splice site nucleotide from the natural group I or group II intron present in the monotron. In some embodiments, the terminal sequence further comprises a natural exon or a fragment thereof and/or a synthetic sequence.

The term “lymphocyte” as used herein includes natural killer (NK) cells, T cells, or B cells. NK cells are a type of cytotoxic (cell toxic) lymphocyte that represent a major component of the innate immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). T cell receptors (TCR) differentiate T cells from other lymphocyte types. The thymus, a specialized organ of the immune system, is the primary site for T cell maturation. There are numerous types of T cells, including: helper T cells (e.g., CD4+ cells), cytotoxic T cells (also known as TC, cytotoxic T lymphocytes, CTL, T-killer cells, cytolytic T cells, CD8+ T cells or killer T cells), memory T cells ((i) stem memory cells (TSCM), like naive cells, are CD45RO−, CCR7+, CD45RA+, CD62L+(L-selectin), CD27+, CD28+ and IL-7Ra+, but also express large amounts of CD95, IL-2R, CXCR3, and LFA-1, and show numerous functional attributes distinctive of memory cells); (ii) central memory cells (TCM) express L-selectin and CCR7, they secrete IL-2, but not IFNγ or IL-4, and (iii) effector memory cells (TEM), however, do not express L-selectin or CCR7 but produce effector cytokines like IFNγ and IL-4), regulatory T cells (Tregs, suppressor T cells, or CD4+CD25+ or CD4+ FoxP3+ regulatory T cells), natural killer T cells (NKT) and gamma delta T cells. B-cells, on the other hand, play a principal role in humoral immunity (with antibody involvement). B-cells make antibodies, are capable of acting as antigen-presenting cells (APCs) and turn into memory B-cells and plasma cells, both short-lived and long-lived, after activation by antigen interaction. In mammals, immature B-cells are formed in the bone marrow.

As used herein, a “miRNA site” or “miRNA binding site” refers to a stretch of nucleotides within a polynucleotide that is capable of forming a duplex with at least 8 nucleotides of a natural miRNA sequence.

As used herein, the terms “monotron,” “monotron sequence,” or “monotron element” are used interchangeably to refer a segment of a precursor RNA polynucleotide that is located at either the 5′ or 3′ end of the polynucleotide, i.e., either 5′ or 3′ from the intervening region. A monotron element refers to a sequence with 70% or higher similarity to a natural group I or group II intron including the splice site dinucleotide. In some embodiments, the monotron is capable of contributing to ribozymatic activity that allows it to enzymatically self-cleave. In some embodiments, the monotron is capable of forming a phosphodiester bond with a terminal sequence, i.e., a sequence containing a splice site dinucleotide and optionally a natural exon sequence or fragment thereof. In some embodiments, the terminal sequence is upstream of the monotron in a linear precursor. In some embodiments, the monotron sequence is upstream of the terminal sequence in a linear precursor. When the terminal sequence is upstream to the monotron in a linear precursor, the monotron can perform two transesterification reactions, e.g., sequentially, self-cleavage and formation of a phosphodiester bond with the terminal sequence. In embodiments in which the terminal sequence is upstream to the monotron in the linear precursor, (a) the monotron is capable of interacting with a nucleophile that is capable of cleaving at the splice site dinucleotide at or near the 5′ end of the monotron, and (b) the cleavage product of (a), i.e., the 5′ splice site nucleotide, e.g., having a 3′ hydroxyl group, engages in a transesterification reaction (cleaves) at the splice site nucleotide of the terminal sequence, yielding a circular RNA or oRNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving at the splice site dinucleotide at or near the 5′ end of the monotron. When the monotron is upstream of the terminal sequence in a linear precursor, the monotron can also perform two transesterification reactions. In embodiments in which the monotron is upstream of the terminal sequence in the linear precursor, (a) the monotron is capable of interacting with a nucleophile that is capable of cleaving at the splice site nucleotide of the terminal element, and (b) the cleavage product of (a), i.e., the 5′ splice site nucleotide, e.g., having a 3′ hydroxyl group, engages in a transesterification reaction (cleaves) at the splice site dinucleotide at or near the 3′ end of the monotron, yielding a circular RNA or oRNA. In these embodiments, the monotron interacts with the nucleophile (e.g., a guanosine, e.g., a free guanosine that is introduced to the precursor) by forming a binding pocket with the nucleophile, and the linear precursor is capable of adopting a conformation in which the nucleophile is in proximity to and is capable of cleaving the splice site nucleotide of the terminal element.

In some embodiments, the monotron comprises a 5′ proximal end of a natural group I or group II intron including the splice site dinucleotide and optionally a natural exon sequence or fragment thereof. In some embodiments, the 5′ end of the monotron refers to nucleotides within the 5′ half of the monotron. In some embodiments, the 3′ end of the monotron refers to nucleotides within the 3′ half of the monotron. In some embodiments, at or near the 5′ end of the monotron refers to within the 5′ half of the monotron. In some embodiments, at or near the 5′ end of the monotron refers to within the first ten 5′ positions in the monotron. In some embodiments, at the 5′ end of the monotron refers to the first 5′ position(s) in the monotron. In some embodiments, at or near the 3′ end of the monotron refers to within the 3′ half of the monotron. In some embodiments, at or near the 3′ end of the monotron refers to within the last ten 3′ positions in the monotron. In some embodiments, at the 3′ end of the monotron refers to last 3′ position(s) in the monotron.

The term “nucleophile” refers to a nucleophilic nucleotide or nucleoside capable of initiating a nucleophilic attack at a splice site and/or transesterification reaction (cleavage) at a splice site.

The term “nucleotide” and “nucleoside” refer to a ribonucleotide, a deoxyribonucleotide, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleosides are similar to nucleotides, e.g., comprising purines and pyrimidines, but without the additional phosphate group.

“Modified” nucleotide or nucleosides, or nucleoside or nucleotide “analogs” include nucleotides or nucleoside having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide or nucleoside modifications are also meant to include nucleotides or nucleoside with bases such as inosine, queuosine, xanthine; sugars such as 2′-methyl ribose; non-natural phosphodiester linkages such as methylphosphonate, phosphorothioate and peptide linkages. The “modified” nucleotide or nucleoside may be naturally occurring (e.g., pseudouridine) or synthetic. Nucleotide or nucleoside modifications include 5-methoxyuridine, 1-methylpseudouridine, and 6-methyladenosine. Exemplary nucleotide or nucleotide modifications are described herein. As exhibited by the exemplary nucleotide or nucleotide modification, such modifications differ from mutations selected from insertions, deletions, addition, or subtraction of nucleotides, for example, the mutations in a permuted Group I and Group II intron segment. As used herein, a nucleotide or nucleoside “comprising no nucleotide or nucleoside modifications” (i.e., comprising 0% modifications) can be interchangeable with “an unmodified nucleotide or nucleoside” in context. A modified polynucleotide sequence contains at least one nucleotide or nucleoside having a modification, e.g., between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides are modified. In some embodiments, “% modification” refers to the level of incorporation within a polynucleotide, i.e., the number of modified nucleotides or nucleosides in a polynucleotide sequence divided by the total number of nucleotides or nucleosides (modified or unmodified) in the polynucleotide sequence. In some embodiments, “% modification” refers to the relative quantity of modified nucleotide or nucleoside used to generate the polynucleotide (e.g., 5% modified adenosine refers to feeding 5 mM modified adenosine and 95 mM unmodified adenosine to generate a polynucleotide sequence).

All nucleotide sequences disclosed herein can represent an RNA sequence or a corresponding DNA sequence. It is understood that deoxythymidine (dT or T) in a DNA is transcribed into a uridine (U) in an RNA. As such, “T” and “U” may be used interchangeably herein in nucleotide sequences.

The terms “nucleic acid”, “polynucleotide”, and “nucleic acid molecule,” are used interchangeably herein to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). For general discussion see The Biochemistry of the Nucleic Acids 5-36, Adams et al., ed., 11th ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Naturally occurring nucleic acids are comprised of nucleotides, including guanine, cytosine, adenine, thymine, and uracil containing nucleotides (G, C, A, T, and U respectively).

As used herein, an “oligonucleotide” is a polynucleotide comprising fewer than 1000 nucleotides, such as a polynucleotide comprising fewer than 500 nucleotides or fewer than 100 nucleotides.

As used herein, “polyA” means a polynucleotide or a portion of a polynucleotide consisting of nucleotides comprising adenine. As used herein, “polyT” means a polynucleotide or a portion of a polynucleotide consisting of nucleotides comprising thymine. As used herein, “polyAC” means a polynucleotide or a portion of a polynucleotide consisting of nucleotides comprising adenine or cytosine.

As used herein, the term “ribosomal skipping element” refers to a nucleotide sequence encoding a short peptide sequence capable of causing generation of two peptide chains from translation of one RNA molecule. While not wishing to be bound by theory, it is hypothesized that ribosomal skipping elements function by (1) terminating translation of the first peptide chain and re-initiating translation of the second peptide chain; or (2) cleavage of a peptide bond in the peptide sequence encoded by the ribosomal skipping element by an intrinsic protease activity of the encoded peptide, or by another protease in the environment (e.g., cytosol).

The terms “sequence identity,” or “sequence similarity” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex regions.

As used here, the term “splicing efficiency” refers to a measurement of the rate of splicing activity (e.g., none, low, or high) in a splicing or self-splicing reaction, for example, in portions of a precursor RNA polynucleotide capable of self-circularization. In some embodiments, the splicing activity of, e.g., a monotron element or intron segment, is affected by the structure and/or sequence of the linear RNA polynucleotide.

As used herein, “structured” with regard to RNA refers to an RNA sequence that is predicted by the RNAFold software or similar predictive tools to form a structure (e.g., a hairpin loop) with itself or other sequences in the same RNA molecule. As used herein, “unstructured” with regard to RNA refers to an RNA sequence that is not predicted by RNA structure predictive tools to form a structure (e.g., a hairpin loop) with itself or other sequences in the same RNA molecule. In some embodiments, unstructured RNA can be functionally characterized using nuclease protection assays.

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

As used herein, “translation initiation element” or “TIE” refers to a portion of the intervening region comprising a sequence to allow translation efficiency of an encoded protein. In some embodiments, core functional elements comprising one or more coding elements will further comprise one or more TIEs. In some embodiments, where the intervening region comprises one or more noncoding elements, the TIE can be part of the noncoding element. In some embodiments, the TIE comprises an internal ribosome entry site (IRES).

As used herein, “transcription” refers to the formation or synthesis of an RNA molecule by an RNA polymerase using a DNA molecule as a template. The disclosure is not limited with respect to the RNA polymerase that is used for transcription. For example, in some embodiments, a T7-type RNA polymerase can be used.

As used herein, “translation” refers to the formation of a polypeptide molecule by a ribosome based upon an RNA template.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

As used herein, the term “transfect” or “transfection” refers to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

As used herein, “transfer vehicle” includes any of the standard pharmaceutical carriers, diluents, excipients, and the like, which are generally intended for use in connection with the administration of biologically active agents, including nucleic acids. In certain embodiments of the present disclosure, the transfer vehicles (e.g., lipid nanoparticles) are prepared to encapsulate one or more materials or therapeutic agents (e.g., circRNA). The process of incorporating a desired therapeutic agent (e.g., circRNA) into a transfer vehicle is referred to herein as or “loading” or “encapsulating” (Lasic, et al., FEBS Lett., 312: 255-258, 1992). The transfer vehicle-loaded or -encapsulated materials (e.g., circRNA) may be completely or partially located in the interior space of the transfer vehicle, within a bilayer membrane of the transfer vehicle, or associated with the exterior surface of the transfer vehicle.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. The treatment or prevention provided by the method disclosed herein can include treatment or prevention of one or more conditions or symptoms of the disease. Also, for purposes herein, “prevention” can encompass delaying the onset of the disease, or a symptom or condition thereof, e.g., prophylaxis of disease.

As used herein, the terms “upstream” and “downstream” refer to relative positions of genetic code, e.g., nucleotides, sequence elements, in polynucleotide sequences. In some embodiments, in an RNA polynucleotide, upstream is toward the 5′ end of the polynucleotide and downstream is toward the 3′ end. In some embodiments, in a DNA polynucleotide, upstream is toward the 5′ end of the coding strand for the gene in question and downstream is toward the 3′ end.

As used herein, a “vaccine” refers to a composition for generating immunity for the prophylaxis and/or treatment of diseases. Accordingly, vaccines are medicaments which comprise antigens and are intended to be used in humans or animals for generating specific defense and protective substances upon administration to the human or animal.

### A. Lipid Definitions

As used herein, the phrase “biodegradable lipid” or “degradable lipid” refers to any of a number of lipid species that are broken down in a host environment on the order of minutes, hours, or days ideally making them less toxic and unlikely to accumulate in a host over time. Common modifications to lipids include ester bonds, and disulfide bonds among others to increase the biodegradability of a lipid.

As used herein, the phrase “biodegradable PEG lipid” or “degradable PEG lipid” refers to any of a number of lipid species where the PEG molecules are cleaved from the lipid in a host environment on the order of minutes, hours, or days ideally making them less immunogenic. Common modifications to PEG lipids include ester bonds, and disulfide bonds among others to increase the biodegradability of a lipid.

As used herein, the term “cationic lipid” or “ionizable lipid” refers to any of a number of lipid species that carry a net positive charge at a selected pH, such as physiological pH 4 and a neutral charge at other pHs such as physiological pH 7.

As used herein, the term “PEG” means any polyethylene glycol or other polyalkylene ether polymer.

As generally defined herein, a “PEG-OH lipid” (also referred to herein as “hydroxy-PEGylated lipid”) is a PEGylated lipid having one or more hydroxyl (—OH) groups on the lipid.

As used herein, a “phospholipid” is a lipid that includes a phosphate moiety and one or more carbon chains, such as unsaturated fatty acid chains.

As used herein, the term “structural lipid” refers to sterols and also to lipids containing sterol moieties. As defined herein, “sterols” are a subgroup of steroids consisting of steroid alcohols.

The terms “head-group” and “tail-group,” when used herein to describe the compounds (e.g., lipids) of the present disclosure, and in particular functional groups that are comprised in such compounds, are used for ease of reference to describe the orientation of such compounds or of one or more functional groups relative to other functional groups. For example, in certain embodiments, a hydrophilic head-group (e.g., guanidinium) is bound (e.g., by one or more of hydrogen-bonds, van der Waals' forces, ionic interactions and covalent bonds) to a cleavable functional group (e.g., a disulfide group), which in turn is bound to a hydrophobic tail-group (e.g., cholesterol). In certain embodiments, the compounds disclosed herein comprise, for example, at least one hydrophilic head-group and at least one hydrophobic tail-group, each bound to at least one cleavable group, thereby rendering such compounds amphiphilic.

As used herein, the term “amphiphilic” means the ability to dissolve in both polar (e.g., water) and non-polar (e.g., lipid) environments. For example, in certain embodiments, the compounds (e.g., lipids) disclosed herein comprise at least one lipophilic tail-group (e.g., cholesterol or a C₆₋₂₀ alkyl) and at least one hydrophilic head-group (e.g., imidazole), each bound to a cleavable group (e.g., disulfide).

As used herein, the term “hydrophilic” is used to indicate in qualitative terms that a functional group is water-preferring, and typically such groups are water-soluble. For example, disclosed herein are compounds (e.g., ionizable lipids) that comprise a cleavable group (e.g., a disulfide (S—S) group) bound to one or more hydrophilic groups (e.g., a hydrophilic head-group), wherein such hydrophilic groups comprise or are selected from the group consisting of imidazole, guanidinium, amino, imine, enamine, an optionally-substituted alkyl amino (e.g., an alkyl amino such as dimethylamino) and pyridyl.

As used herein, the term “hydrophobic” is used to indicate in qualitative terms that a functional group is water-avoiding, and typically such groups are not water soluble. In certain embodiments, at least one of the functional groups of moieties that comprise the compounds disclosed herein is hydrophobic in nature (e.g., a hydrophobic tail-group comprising a naturally occurring lipid such as cholesterol). For example, disclosed herein are compounds (e.g., ionizable lipids) that comprise a cleavable functional group (e.g., a disulfide (S—S) group) bound to one or more hydrophobic groups, wherein such hydrophobic groups may comprise, or may be selected from, one or more naturally occurring lipids such as cholesterol, an optionally substituted, variably saturated or unsaturated C₆-C₂₀ alkyl, and/or an optionally substituted, variably saturated or unsaturated C₆-C₂₀ acyl.

As used herein, the term “liposome” generally refers to a vesicle composed of lipids (e.g., amphiphilic lipids) arranged in one or more spherical bilayer or bilayers. Such liposomes may be unilamellar or multilamellar vesicles which have a membrane formed from a lipophilic material and an aqueous interior that contains the encapsulated circRNA to be delivered to one or more target cells, tissues and organs.

As used herein, the phrase “lipid nanoparticle” or “LNP” refers to a transfer vehicle comprising one or more cationic or ionizable lipids, stabilizing lipids, structural lipids, and helper lipids.

In certain embodiments, the compositions described herein comprise one or more liposomes or lipid nanoparticles. Examples of suitable lipids (e.g., ionizable lipids) that may be used to form the liposomes and lipid nanoparticles contemplated include one or more of the compounds disclosed herein (e.g., HGT4001, HGT4002, HGT4003, HGT4004 and/or HGT4005). Such liposomes and lipid nanoparticles may also comprise additional ionizable lipids such as C12-200, dLin-KC2-DMA, and/or HGT5001, helper lipids, structural lipids, PEG-modified lipids, MC3, DLinDMA, DLinkC2DMA, cKK-E12, ICE, HGT5000, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLincarbDAP, DLinCDAP, kLin-K-DMA, dLin-K-XTC2-DMA, HGT4003, and combinations thereof.

In some embodiments, a lipid, e.g., an ionizable lipid, disclosed herein comprises one or more cleavable groups. The terms “cleave” and “cleavable” are used in this regard to mean that one or more chemical bonds (e.g., one or more of covalent bonds, hydrogen-bonds, van der Waals' forces and/or ionic interactions) between atoms in or adjacent to the subject functional group are broken (e.g., hydrolyzed) or are capable of being broken upon exposure to selected conditions (e.g., upon exposure to enzymatic conditions). In certain embodiments, the cleavable group is a disulfide functional group, and in particular embodiments is a disulfide group that is capable of being cleaved upon exposure to selected biological conditions (e.g., intracellular conditions). In certain embodiments, the cleavable group is an ester functional group that is capable of being cleaved upon exposure to selected biological conditions. For example, the disulfide groups may be cleaved enzymatically or by a hydrolysis, oxidation or reduction reaction. Upon cleavage of such disulfide functional group, the one or more functional moieties or groups (e.g., one or more of a head-group and/or a tail-group) that are bound thereto may be liberated. Exemplary cleavable groups may include, but are not limited to, disulfide groups, ester groups, ether groups, and any derivatives thereof (e.g., alkyl and aryl esters). In certain embodiments, the cleavable group is not an ester group or an ether group. In some embodiments, a cleavable group is bound (e.g., bound by one or more of hydrogen-bonds, van der Waals' forces, ionic interactions and covalent bonds) to one or more functional moieties or groups (e.g., at least one head-group and at least one tail-group). In certain embodiments, at least one of the functional moieties or groups is hydrophilic (e.g., a hydrophilic head-group comprising one or more of imidazole, guanidinium, amino, imine, enamine, optionally-substituted alkyl amino and pyridyl).

### B. Chemical Definitions

The disclosure may include compounds and pharmaceutically acceptable salts thereof, pharmaceutical compositions containing such compounds and methods of using such compounds and compositions, and the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “aliphatic” or “aliphatic group,” means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule or multiple points of attachment to the rest of the molecule, as would be readily apparent to a person of ordinary skill in the art based on the context of the described molecule. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic, bicyclic, or polycyclic C₃-C₁₄ hydrocarbon that is completely saturated or that contains one or more units of unsaturation, but which is not aromatic, that has a single point of attachment to the rest of the molecule. Exemplary aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. Examples of bicyclic and polycyclic cycloalkyls include bridged, fused, and spirocyclic carbocyclyls.

As used herein, the term “alkyl” refers to both straight and branched chain C₁₋₄₀ hydrocarbons (e.g., C₆₋₂₀ hydrocarbons), and include both saturated and unsaturated hydrocarbons. In certain embodiments, the alkyl may comprise one or more cyclic alkyls and/or one or more heteroatoms such as oxygen, nitrogen, or sulfur and may optionally be substituted with substituents (e.g., one or more of alkyl, halo, alkoxyl, hydroxy, amino, aryl, ether, ester or amide). In certain embodiments, a contemplated alkyl includes (9Z,12Z)-octadeca-9,12-dien. The use of designations such as, for example, “C₆₋₂₀” is intended to refer to an alkyl (e.g., straight or branched chain and inclusive of alkenes and alkyls) having the recited range carbon atoms. In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂-8 alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋ₛ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene” group, it is understood that the range or number refers to the range or number of carbons in the linear carbon divalent chain. “Alkylene,” “alkenylene,” and “alkynylene” groups may be substituted or unsubstituted with one or more substituents as described herein.

The term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term comprises any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

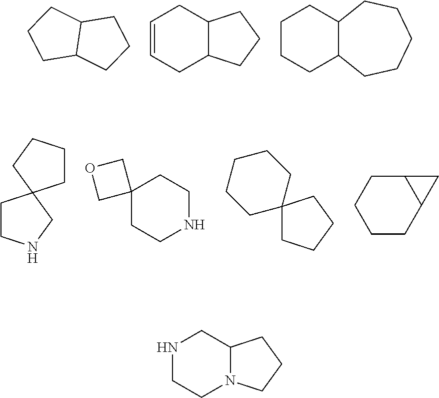

Exemplary bridged bicyclics include:

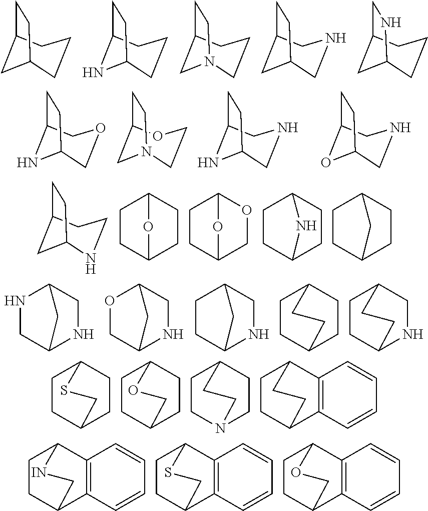

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈ cycloalkyl,” derived from a cycloalkane. Exemplary cycloalkyl groups include, but are not limited to, cyclohexanes, cyclopentanes, cyclobutanes and cyclopropanes.

As used herein, “cyano” refers to —CN.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

The terms “halo” and “halogen” as used herein refer to an atom selected from fluorine (fluoro, F), chlorine (chloro, Cl), bromine (bromo, Br), and iodine (iodo, I). In certain embodiments, the halo group is either fluoro or chloro.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. Illustrative substituents include, but are not limited to, halogens, hydroxyl groups, or any other organic groupings containing any number of carbon atoms, for example, 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; P(O)₂R^{∘}; P(O)R^{∘}₂; OP(O)R^{∘}₂; OP(O)(OR^{∘})₂; —SiR^{∘}₃; —(C₁₋₄ straight or branched alkylene)O—N(R^{∘})₂; or (C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Heteroatoms such as nitrogen may have hydrogen substituents and/or any permissible substituents of organic compounds described herein which satisfy the valences of the heteroatoms. It is understood that “substitution” or “substituted” comprises the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In typical embodiments, the present disclosure is intended to encompass the compounds disclosed herein, and the pharmaceutically acceptable salts, pharmaceutically acceptable esters, tautomeric forms, polymorphs, and prodrugs of such compounds. In some embodiments, the present disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

In certain embodiments, the compounds (e.g., ionizable lipids) and the transfer vehicles (e.g., lipid nanoparticles) of which such compounds are a component exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein.

It is to be understood that the terminology used herein is for the purpose of describing particular embodiments only, and is not intended to be limiting. Unless specifically stated or obvious from context, as used herein, the term “or” is understood to be inclusive. Unless defined herein and below in the reminder of the specification, all technical and scientific terms used herein have the same meaning as commonly understood by one of ordinary skill in the art.

### 2. Circular RNA

Provided herein are RNA constructs, e.g., circular RNA constructs, encoding a CD19 binding molecule, e.g., CAR, and related pharmaceutical compositions comprising transfer vehicles, wherein the RNA constructs are capable of in vivo delivery to immune cells for therapy. According to the present disclosure, the RNA constructs, e.g., circular RNA constructs, provided herein can be injected into an animal (e.g., a human), such that a polypeptide encoded by the RNA is expressed inside the animal, for example by one or more immune cells, e.g., T cells, NK cells, macrophages, etc.

In some embodiments, the CD19 binding molecule may be encoded by linear RNA (mRNA) constructs, and pharmaceutical compositions comprising transfer vehicles and said linear mRNA constructs are capable of in vivo delivery to immune cells for therapy.

In some embodiments, provided herein are circular RNA comprising, optionally in the following order, a 3′ self-spliced exon segment, optionally a first spacer, a translation initiation element (TIE) (e.g., comprising an Internal Ribosome Entry Site (IRES)), an expression sequence encoding a CD19 binding molecule (e.g., encoding a chimeric antigen receptor (CAR)), optionally a second spacer, and a 5′ self-spliced exon segment.

In some embodiments, the circular RNA and/or linear mRNA comprises expression sequences encoding two or more CD19 binding molecules. In some embodiments, the two or more CD19 molecules are two or more copies of the same CD19 binding molecule. In some embodiments, the two or more CD19 molecules are two or more copies of different CD19 binding molecules. In some embodiments, the two or more CD19 molecules are different CD19 binding molecules. In some embodiments, a first CD19 binding molecule comprises a first set of complementary determining region sequences (CDRs), and a second CD19 binding molecule comprises a second set of CDRs that differs from the first set of CDRs; in these embodiments, the first set of CDRs and the second set of CDRs may bind to the same or to different epitopes of CD19. In some embodiments, a first CD19 binding molecule binds a first epitope of a CD19 antigen, and a second CD19 binding molecule binds a second epitope of CD19 that differs from the first epitope. In some embodiments, the circular RNA comprises expression sequences that in conjunction encodes for a single CD19 binding molecule.

In certain embodiments, the RNA, e.g., circular RNA and/or linear mRNA, is formulated into a pharmaceutical composition. In certain embodiments, the pharmaceutical composition comprises a transfer vehicle. In certain embodiments, a circular RNA and/or linear mRNA construct comprising a TIE and at least one expression sequence encoding a CD19 binding molecule is formulated into a pharmaceutical composition comprising a transfer vehicle.

In certain embodiments, pharmaceutical compositions comprising a circular RNA and/or linear mRNA construct comprising a TIE and at least one expression sequence encoding a CD19 binding molecule, and a transfer vehicle are disclosed. In certain embodiments, the transfer vehicle facilitates and/or enhances the delivery and release of circular RNA and/or linear mRNA to one or more target cells.

In certain embodiments, the circular RNA and/or linear mRNA constructs and related pharmaceutical compositions comprise a TIE and at least one expression sequence encoding a CD19 binding molecule, wherein the TIE is capable of facilitating expression of the protein when delivered in vivo.

In some embodiments, a polynucleotide encodes a protein that is made up of subunits that are encoded by more than one gene. For example, the protein may be a heterodimer, wherein each chain or subunit of the protein is encoded by a separate gene. It is possible that more than one circular RNA and/or linear mRNA molecule is delivered in the transfer vehicle and each circular RNA and/or linear mRNA encodes a separate subunit of the protein. Alternatively, a single circular RNA may be engineered to encode more than one subunit (e.g., subunits of a CD19 binding molecule). In certain embodiments, separate circular RNA and/or linear mRNA molecules encoding the individual subunits may be administered in separate transfer vehicles.

In certain embodiments, the circular RNA and/or linear mRNA comprises a TIE and at least one expression sequence encoding an anti-CD19 CAR construct. In some embodiments, the CAR may be programmed to both recognize a CD19 antigen and, when bound to that CD19 antigen, activate the immune cell to attack and destroy the cell. In certain embodiments, the payload encoded by the circular RNA and/or linear mRNA polynucleotide may be optimized through use of a specific internal ribosome entry sites (IRES) within the TIE. The TIE can comprise an untranslated region (UTR), aptamer complex, or a combination thereof. The UTR can be in whole or in part from a viral or eukaryotic mRNA. In some embodiments, TIE, e.g., IRES, specificity within a circular RNA can significantly enhance expression of the CD19 binding molecule encoded within the coding element.

The circular RNA is produced by transcription of a DNA template that results in formation of a precursor linear RNA polynucleotide capable of circularizing. Linear precursor RNA polynucleotides are provided for producing circular RNA constructs and related pharmaceutical compositions. The DNA template shares the same sequence as the precursor linear RNA polynucleotide prior to splicing of the precursor linear RNA polynucleotide. The DNA template shares the same sequence as the precursor linear RNA polynucleotide prior to splicing of the precursor linear RNA polynucleotide. In some embodiments, said linear precursor RNA polynucleotide undergoes splicing to remove a 3′ intron element and 5′ intron element during the process of circularization. In some embodiments, the resulting circular RNA polynucleotide lacks a 3′ intron element and a 5′ intron element, but maintains a 3′ exon element, an intervening region comprising an anti-CD19 CAR coding sequence, and a 5′ exon element. Circularization strategies are known in the art and described elsewhere herein. In certain embodiments, the resulting circular RNA can include a PIE (permuted intron-exon) region, a translation region (IRES and coding/noncoding elements), and a PIE region. The resulting permuted intron-exon (PIE) regions allow for 5′ and 3′ ends of the RNA to covalently link and form the circular RNA.

In certain embodiments circular RNA and/or linear mRNA provided herein is produced inside a cell. In some embodiments, linear precursor RNA, e.g., for making circular RNA, is transcribed using a DNA template (e.g., in some embodiments, using a vector provided herein) in the cytoplasm by a bacteriophage RNA polymerase, or in the nucleus by host RNA polymerase II and then circularized.

In certain embodiments, the circular RNA and/or linear mRNA provided herein is injected into an animal (e.g., a human, non-human primate, or rodent), such that the CD19 binding molecule, e.g., CAR, encoded by the circular RNA and/or linear mRNA molecule is expressed inside the animal. In certain embodiments, the CD19 binding molecule, e.g., CAR, encoded by the circular RNA and/or linear mRNA is embedded at least in part on the surface or inside of a cell within the animal.

In some embodiments, the DNA (e.g., vector), linear RNA (e.g., precursor linear RNA for making circular RNA), linear mRNA, and/or circular RNA polynucleotide provided herein is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length. In some embodiments, the polynucleotide is at least 300 nucleotides, 400 nucleotides, 500 nucleotides, 600 nucleotides, 700 nucleotides, 800 nucleotides, 900 nucleotides, 1000 nucleotides, 1100 nucleotides, 1200 nucleotides, 1300 nucleotides, 1400 nucleotides, 1500 nucleotides, 2000 nucleotides, 2500 nucleotides, 3000 nucleotides, 3500 nucleotides, 4000 nucleotides, 4500 nucleotides, or 5000 nucleotides in length. In some embodiments, the polynucleotide is no more than 3000 nucleotides, 3500 nucleotides, 4000 nucleotides, 4500 nucleotides, 5000 nucleotides, 6000 nucleotides, 7000 nucleotides, 8000 nucleotides, 9000 nucleotides, or 10000 nucleotides in length. In some embodiments, the length of a DNA, linear RNA, and/or circular RNA polynucleotide provided herein is about 300 nucleotides, 400 nucleotides, 500 nucleotides, 600 nucleotides, 700 nucleotides, 800 nucleotides, 900 nucleotides, 1000 nucleotides, 1100 nucleotides, 1200 nucleotides, 1300 nucleotides, 1400 nucleotides, 1500 nucleotides, 2000 nucleotides, 2500 nucleotides, 3000 nucleotides, 3500 nucleotides, 4000 nucleotides, 4500 nucleotides, 5000 nucleotides, 6000 nucleotides, 7000 nucleotides, 8000 nucleotides, 9000 nucleotides, or 10000 nucleotides.

In some embodiments, the circular RNA polynucleotide provided herein has higher functional stability than linear mRNA comprising the same expression sequence. In some embodiments, the circular RNA provided herein has higher functional stability than linear mRNA comprising the same expression sequence, modified nucleotides (e.g., 5moU modifications), an optimized UTR, a cap, and/or a polyA tail. In some embodiments, the circular RNA provided herein has greater durability than linear mRNA comprising the same expression sequence.

In some embodiments, the circular RNA polynucleotide provided herein has a functional half-life of at least 5 hours, 10 hours, 15 hours, 20 hours, 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of a linear mRNA polynucleotide comprising the same expression sequence. In some embodiments, functional half-life can be assessed through the detection of functional protein synthesis.

In some embodiments, the circular RNA polynucleotide provided herein has a half-life of at least 5 hours, 10 hours, 15 hours, 20 hours, 30 hours, 40 hours, 50 hours, 60 hours, 70 hours, or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of a linear mRNA polynucleotide comprising the same expression sequence. In some embodiments, the circular RNA polynucleotide, or pharmaceutical composition thereof, has a functional half-life in a human cell greater than or equal to that of a pre-determined threshold value. In some embodiments the functional half-life is determined by a functional protein assay. For example, in some embodiments, the functional half-life is determined by an in vitro luciferase assay, wherein the activity of Gaussia luciferase (GLuc) is measured in the media of human cells (e.g. HepG2) expressing the circular RNA polynucleotide every 1, 2, 6, 12, or 24 hours over 1, 2, 3, 4, 5, 6, 7, or 14 days. In other embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear mRNA polynucleotide comprising the same expression sequence as the circular RNA polynucleotide.

In some embodiments, the circular RNA provided herein may have a higher magnitude of expression than linear mRNA comprising the same expression sequence, e.g., a higher magnitude of expression 24 hours after administration of RNA to cells. In some embodiments, the circular RNA provided herein has a higher magnitude of expression than linear mRNA comprising the same expression sequence, 5 moU modifications, an optimized UTR, a cap, and/or a polyA tail. In some embodiments, the circular RNA provided herein has a greater duration of expression than that of a linear mRNA polynucleotide comprising the same expression sequence. In some embodiments, the circular RNA provided herein has a greater duration of expression of a CAR (e.g., CD19 CAR) than that of a linear mRNA polynucleotide comprising the same expression sequence. In some embodiments, the circular RNA provided herein has a greater duration of expression of a CAR (e.g., CD19 CAR) in immune cells (e.g., T cells) than that of a linear mRNA polynucleotide comprising the same expression sequence.

In some embodiments, the circular RNA provided herein may be less immunogenic than a linear mRNA comprising the same expression sequence when exposed to an immune system of an organism or a certain type of immune cell. In some embodiments, the circular RNA provided herein is associated with modulated production of cytokines when exposed to an immune system of an organism or a certain type of immune cell. For example, in some embodiments, the circular RNA provided herein is associated with reduced production of IFN-β1, RIG-I, IL-2, IL-6, IFNγ, and/or TNFα when exposed to an immune system of an organism or a certain type of immune cell as compared to linear mRNA comprising the same expression sequence. In some embodiments, the circular RNA provided herein is associated with less IFN-β1, RIG-I, IL-2, IL-6, IFNγ, and/or TNFα transcript induction when exposed to an immune system of an organism or a certain type of immune cell as compared to linear mRNA comprising the same expression sequence. In some embodiments, the circular RNA provided herein is less immunogenic than linear mRNA comprising the same expression sequence. In some embodiments, the circular RNA provided herein is less immunogenic than linear mRNA comprising the same expression sequence, modified nucleotides (e.g., 5moU modifications), an optimized UTR, a cap, and/or a polyA tail. In some embodiments, the circular RNA provided herein has greater functionality, and/or exhibits greater cytotoxicity levels in an immune cells, and/or exhibits greater B cell depletion when expressed, and/or exhibits greater B cell depletion over time and/or over a longer period of time when expressed, and/or exhibits greater B cell depletion in the blood (e.g., peripheral blood), spleen, bone marrow and/or lymph nodes (e.g., mesenteric lymph nodes and/or pooled draining lymph nodes) when expressed as compared to linear mRNA comprising the same expression sequence. In some embodiments, the circular RNA encoding a CD19 binding molecule (e.g., CD19 CAR) has greater functionality than linear mRNA comprising the same expression sequence. In some embodiments, the circular RNA encoding a CD19 binding molecule (e.g., CD19 CAR) has greater cytotoxicity levels in an immune cell compared to linear mRNA comprising the same expression sequence. In some embodiments, the circular RNA encoding a CD19 binding molecule (e.g., CD19 CAR) comprises greater B cell depletion when expressed compared to linear mRNA comprising the same expression sequence. In some embodiments, the circular RNA encoding a CD19 binding molecule (e.g., CD19 CAR) comprises greater B cell depletion over time and/or over a longer period of time when expressed compared to linear mRNA comprising the same expression sequence. In some embodiments, the circular RNA encoding a CD19 binding molecule (e.g., CD19 CAR) comprises greater B cell depletion in the blood (e.g., peripheral blood), spleen, bone marrow and/or lymph nodes (e.g., mesenteric lymph nodes and/or pooled draining lymph nodes) when expressed compared to linear mRNA comprising the same expression sequence.

In some embodiments, the circular RNA and/or linear mRNA provided herein can be encapsulated by a transfer vehicle (e.g., LNPs), which can deliver these RNA constructs. Encapsulating the circular RNA and/or linear mRNA in the transfer vehicle, for example can efficiently introduce the CD19 binding molecule, e.g., CAR, genes to immune cells, e.g., T cells. The transfer vehicles can comprise, e.g., ionizable lipids, PEG-modified lipids, helper lipids, and/or structural lipids, that are capable of encapsulating the circular RNAs. Pharmaceutical compositions are provided for circular RNA constructs comprising an IRES, an expression sequence encoding a CD19 binding molecule (e.g., CD19 CAR), and a transfer vehicle.

In certain embodiments, the circular RNA and/or linear mRNA constructs provided herein can be transfected into a cell as is or can be transfected in DNA vector form and transcribed in the cell. Transcription of circular RNA and/or linear mRNA from a transfected DNA vector can be via added polymerases or polymerases encoded by nucleic acids transfected into the cell, or preferably via endogenous polymerases. Accordingly, also provided herein is a eukaryotic cell comprising a circular RNA and/or linear mRNA polynucleotide provided herein. In some embodiments, the eukaryotic cell is a human cell. In some embodiments, the eukaryotic cell is an immune cell. In some embodiments, the eukaryotic cell is a T cell, natural killer cell (NK cell), dendritic cell, macrophage, B cell, neutrophil, or basophil. Also provided herein is a prokaryotic cell comprising a circular RNA and/or linear mRNA polynucleotide provided herein.

In some embodiments, provided herein is a T cell, e.g., human T cell, comprising the circular RNA and/or linear mRNA constructs provided herein. In some embodiments, provided herein is a helper T cell, e.g., human helper T cell, comprising the circular RNA and/or linear mRNA constructs provided herein. In some embodiments, provided herein is a cytotoxic T cell, e.g., human cytotoxic T cell, comprising the circular RNA and/or linear mRNA constructs provided herein. In some embodiments, provided herein is a NK cell, e.g., human NK cell, comprising the circular RNA and/or linear mRNA constructs provided herein. In some embodiments, provided herein is a macrophage, e.g., human macrophage, comprising the circular RNA and/or linear mRNA constructs provided herein. In some embodiments, provided herein is a monocyte, e.g., human monocyte, comprising the circular RNA and/or linear mRNA constructs provided herein. In some embodiments, provided herein is a myeloid cell, human monocyte, comprising the circular RNA and/or linear mRNA constructs provided herein. In some embodiments, these cells are present in the bone marrow. In some embodiments, these cells are present in the spleen. In some embodiments, these cells are present in the blood, e.g., peripheral blood.

In some embodiments, provided herein is a CD3+ cell, e.g., human CD3+ cell, comprising the circular RNA and/or linear mRNA constructs provided herein. In some embodiments, provided herein is a CD4+ cell, e.g., human CD4+ cell, comprising the circular RNA and/or linear mRNA constructs provided herein. In some embodiments, provided herein is a CD8+ cell, e.g., human CD8+ cell, comprising the circular RNA and/or linear mRNA constructs provided herein. In some embodiments, provided herein is a CD14+ cell, e.g., human CD14+ cell, comprising the circular RNA and/or linear mRNA constructs provided herein. In some embodiments, provided herein is a CD16+ cell, e.g., human CD16+ cell, comprising the circular RNA and/or linear mRNA constructs provided herein. In some embodiments, provided herein is a CD56+ cell, e.g., human CD56+ cell, comprising the circular RNA and/or linear mRNA constructs provided herein. In some embodiments, provided herein is a CD11B+ cell, e.g., human CD11B+ cell, comprising the circular RNA and/or linear mRNA constructs provided herein. In some embodiments, provided herein is a CD33+ cell, e.g., human CD33+ cell, comprising the circular RNA and/or linear mRNA constructs provided herein. In some embodiments, provided herein is a CD33+ CD14+ cell, e.g., human CD33+ CD14+ cell, comprising the circular RNA and/or linear mRNA constructs provided herein. In some embodiments, provided herein is a CD33+ CD14+ cell, e.g., human CD33+ CD64+ cell, comprising the circular RNA and/or linear mRNA constructs provided herein. In some embodiments, these cells are present in the bone marrow. In some embodiments, these cells are present in the spleen. In some embodiments, these cells are present in the blood, e.g., peripheral blood.

The circular RNA and/or linear mRNA can be unmodified, partially modified or completely modified. In one embodiment, the circular RNA and/or linear mRNA contains at least one nucleoside modification. In one embodiment, up to 100% of the nucleosides of the circular RNA and/or linear mRNA are modified. In one embodiment, at least one nucleoside modification is a uridine modification or an adenosine modification. In one embodiment, at least one nucleoside modification is selected from N6-methyladenosine (m6A), pseudouridine (ψ), N1-methylpseudouridine (m1ψ), and 5-methoxyuridine (5moU). In one embodiment, the precursor RNA is modified with methylpseudouridine (m1ψ). In certain embodiments, a coding sequence (e.g., encoding a CD19 binding molecule) of the circular RNA and/or linear mRNA contains at least one nucleoside modification. In certain embodiments, a coding sequence (e.g., encoding a CD19 binding molecule) of a circular RNA and/or linear mRNA does not contain any nucleotide modifications.

In certain embodiments, a provided polynucleotide (e.g., a DNA template, a linear precursor RNA for making circular RNA, a linear mRNA polynucleotide, or a circular RNA polynucleotide) comprises modified nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2G (N²,N²-dimethylguanosine); m²Gm (N²,2′-O-dimethylguanosine); m²₂Gm (N²,N²,2′-O-trimethylguanosine); Gr(p) (2′-O-ribosylguanosine(phosphate)); yW (wybutosine); o₂yW (peroxywybutosine); OHyW (hydroxywybutosine); OHyW* (undermodified hydroxy wybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G+(archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ 2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine. In some embodiments, such modified nucleosides provide additional stability and resistance to immune activation.

In some embodiments, provided herein is a circular RNA polynucleotide comprising, in the following order, a 3′ self-spliced exon segment, a translation initiation element (TIE), an expression sequence encoding a CD19 binding molecule with which the TIE is not naturally associated, and a 5′ self-spliced exon segment.

In some embodiments, provided herein is a circular RNA polynucleotide comprising: i) a 5′ combined accessory element; ii) an intervening region; and iii) a 3′ combined accessory element, where the intervening region is between the 5′ combined accessory element and the 3′ combined accessory element.

In some embodiments, the 5′ combined accessory element comprises a 3′ self-spliced exon segment. In some embodiments, the 3′ self-spliced exon segment comprises an exon segment or fragment thereof. In some embodiments, the 3′ self-spliced exon segment comprises a 3′ nucleotide of a 3′ splice site dinucleotide. In some embodiments, the 3′ self-spliced exon segment comprises an exon segment and a 3′ nucleotide of a 3′ splice site dinucleotide. In some embodiments, the exon segment comprises a natural exon sequence or non-naturally occurring sequence. In some embodiments, the 3′ splice site dinucleotides are distinct from the natural splice site dinucleotide(s) associated with a natural Group I or Group II intron sequence.

In some embodiments, the 3′self-spliced exon segment comprises a sequence having a percent sequence identity of about 70%, 75%, 80%, 85%, 90%, 91%, 92%, 93%, 94%, 95%, 96%, 97%, 98%, 99% or more to a sequence selected from Table 2 herein, Table 3 herein, or PCT/US2024/027627, the contents of which are hereby incorporated by reference. In some embodiments, the 3′ self-spliced exon segment is selected from an exon segment disclosed in Table 2 herein, Table 3 herein, or PCT/US2024/027627, the contents of which are hereby incorporated by reference. In some embodiments, the self-spliced exon segment is, e.g., 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table 2 or is e.g., 5 nucleotides, 6 nucleotides, 7 nucleotides, 8 nucleotides, 9 nucleotides, or 10 nucleotides from the exonic sequences of Table 3. See also PCT/US2024/027627, the contents of which are hereby incorporated by reference.

In some embodiments, the 3′ combined accessory element comprises a 5′ self-spliced exon segment. In some embodiments, the 5′ self-spliced exon segment comprises an exon segment or fragment thereof. In some embodiments, the 5′ self-spliced exon segment comprises a 5′ nucleotide of a 5′ splice site dinucleotide. In some embodiments, the 5′ self-spliced exon segment comprises an exon segment and a 5′ nucleotide of a 5′ splice site dinucleotide. In some embodiments, the exon segment comprises a natural exon sequence or non-naturally occurring sequence. In some embodiments, the 5′splice site dinucleotides are distinct from the natural splice site dinucleotide(s) associated with a natural Group I or Group II intron sequence.

In some embodiments, the 5′self-spliced exon segment comprises a sequence having a percent sequence identity of about 70%, 75%, 80%, 85%, 90%, 91%, 92%, 93%, 94%, 95%, 96%, 97%, 98%, 99% or more to a sequence selected from Table 2 herein, Table 3 herein, or PCT/US2024/027627, the contents of which are hereby incorporated by reference. In some embodiments, the 5′ self-spliced exon segment is selected from an exon segment in Table 2 herein, Table 3 herein, or PCT/US2024/027627, the contents of which are hereby incorporated by reference. In some embodiments, the self-spliced exon segment is e.g., 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table 2 or is e.g., 5 nucleotides, 6 nucleotides, 7 nucleotides, 8 nucleotides, 9 nucleotides, or 10 nucleotides from the exonic sequences of Table 3. See also PCT/US2024/027627, the contents of which are hereby incorporated by reference.

In some embodiments, as set forth herein, the intervening region comprises an expression sequence (coding region or element) encoding a CD19 binding molecule. In some embodiments, the intervening region comprises at least one translation initiation element (TIE). In some embodiments, the TIE comprises a viral or eukaryotic internal ribosome entry site (IRES). In some embodiments, the IRES comprises a sequence selected from the sequences in Table 4 or a fragment thereof or a sequence from PCT/US2022/033091 or PCT/US2023/084046, the contents of which are hereby incorporated by reference. In some embodiments the TIE is operably linked to an expression sequence encoding CD19 with which the TIE is not naturally associated.

In some embodiments, the intervening region comprises an untranslated region (UTR). In some embodiments, the UTR comprises one or more noncoding elements. In some embodiments, the one or more noncoding elements are selected from, e.g., a natural 3′ Untranslated Region (UTR), a natural 5′ Untranslated Region (UTR), a synthetic spacer sequence, an aptamer, and lncRNA, miRNA, and a miRNA sponge. In some embodiments, the noncoding element is or comprises the TIE.

In some embodiments, the intervening region comprises an expression sequence encoding a CD19 binding molecule, e.g., CAR. In some embodiments, the expression sequence encodes two or more polypeptides. In some embodiments, the expression sequence comprises one or more expression sequences or portions thereof, e.g., Table 2.

In some embodiments, provided herein are circular RNA polynucleotides comprising, in the following order, i) a 5′ combined accessory element comprising a 3′ self-spliced exon segment; ii) an intervening region comprising an expression sequence encoding a CD19 binding molecule; and iii) a 3′ combined accessory element comprising a 5′ self-spliced exon segment. In some embodiments, the 3′ self-spliced exon segment and/or the 5′ self-spliced exon segment is selected from an exon segment disclosed herein, e.g., in Table 2 or Table 3, or PCT/US2024/027627.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment; ii) an intervening region encoding a CD19 binding molecule; and iii) a 3′ combined accessory element comprising a 5′ self-spliced exon segment, wherein the 5′ self-spliced exon segment comprises an exon segment. In some embodiments, the 3′ self-spliced exon segment and/or the 5′ self-spliced exon segment is selected from an exon segment disclosed herein, e.g., in Table 2 or Table 3, or PCT/US2024/027627.

In some embodiments, provided herein are circular RNA polynucleotides comprising, in the following order, i) a 5′ combined accessory element comprising a 3′ self-spliced exon segment, wherein the 3′ self-spliced exon segment comprises an exon segment and a 3′ nucleotide of a 3′ splice site dinucleotide; ii) an intervening region encoding a CD19 binding molecule; and iii) a 3′ combined accessory element comprising a 5′ self-spliced exon segment, wherein the 5′ self-spliced exon segment comprises an exon segment and a 5′ nucleotide of a 5′ splice site dinucleotide. In some embodiments, the 3′ self-spliced exon segment and/or the 5′ self-spliced exon segment is selected from an exon segment disclosed herein, e.g., in Table 2 or Table 3, or PCT/US2024/027627.

A circular RNA polynucleotide comprising, in the following order, a 3′ self-spliced exon segment, an intervening region comprising an expression sequence encoding a CD19 binding molecule, and a 5′ self-spliced exon segment, wherein at least one of the 3′ or 5′ self-spliced exon segments is selected from an exon segment comprising a sequence selected from Table 2, Table 3, or PCT/US2024/027627.

As a non-limiting example, a circular RNA polynucleotide comprises the following elements operably connected and arranged in the following sequence:
(a) a 3′ exon segment comprising a Group I or Group II exon 3′ nucleotide of a 3′ splice site dinucleotide;
(b) an intervening region comprising an expression sequence encoding a CD19 binding molecule; and
(c) a 5′ exon segment comprising a Group I or Group II exon 5′ nucleotide of a 5′ splice site dinucleotide.

As set forth in detail herein, in some embodiments, a circular RNA polynucleotide comprises a retained portion of a monotron element. See, e.g., supra. In some embodiments, a circular RNA polynucleotide comprises: a 5′ internal spacer, a 5′ internal duplex, at least a portion of a terminal element (or sequence or segment), at least a portion of a monotron element (or sequence or segment), a 3′ internal duplex, a 3′ internal spacer, an intervening region comprising an expression sequence encoding a CD19 binding molecule. In some embodiments, the intervening region further comprises TIE, e.g., operably linked to the expression sequence. In some embodiments, the monotron element present in the precursor RNA polynucleotide, of which a portion is retained in the circular RNA polynucleotide, comprises a polynucleotide sequence that has a percent sequence identity of about 70%, 75%, 80%, 85%, 90%, 91%, 92%, 93%, 94%, 95%, 96%, 97%, 98%, 99% or more to a sequence selected from PCT/US2024/027627.

In some embodiments, the circular RNA polynucleotide comprises the following elements operably connected and arranged in the following sequence:
(a) a 5′ internal spacer,
(b) a 5′ internal duplex,
(c) at least a portion of a terminal element,
(d) at least a portion of a monotron element,
(e) a 3′ internal duplex,
(f) a 3′ internal spacer, and
(g) an intervening region, optionally comprising a coding region, and IRES.

In some embodiments, the circular RNA polynucleotide comprises the following elements operably connected and arranged in the following sequence:
(a) a 5′ internal spacer,
(b) a 5′ internal duplex,
(c) at least a portion of a monotron element,
(d) at least a portion of a terminal element,
(e) a 3′ internal duplex,
(f) a 3′ internal spacer, and
(g) an intervening region, optionally comprising a coding region, and IRES.

As a further non-limiting example, a circular RNA polynucleotide comprises the following elements operably connected and arranged in the following sequence:
(a) at least a portion of a terminal element,
(b) a 3′ exon segment comprising a 3′ nucleotide of a 3′ splice site dinucleotide,
(c) an intervening region,
(d) a 5′ exon segment comprising a 5′ nucleotide of a 5′ splice site dinucleotide, and
(e) at least a portion of a monotron element;
wherein the 5′ and/or 3′ splice site dinucleotides are distinct from the natural splice site dinucleotide(s) associated with a natural Group I or Group II intron sequence.

In some embodiments, element (d) comprises the first nucleotide of a 5′ Group I or Group II splice site dinucleotide and a natural exon sequence. In some embodiments, element (b) comprises the second nucleotide of a 3′ Group I or Group II exon splice site dinucleotide and a natural exon sequence.

In some embodiments, in the circular RNA polynucleotide, the 5′ exon element comprises the second nucleotide of a 3′ Group I or Group II exon splice site dinucleotide and a natural exon sequence. In some embodiments, the 3′ exon element fragment comprises the first nucleotide of a 5′ Group I or Group II splice site dinucleotide and a natural exon sequence. In some embodiments, the 5′ exon element comprises a 5′ internal duplex; and the 3′ exon element comprises a 3′ internal duplex. In some embodiments, the 5′ exon element comprises a 5′ internal spacer. In some embodiments, the 3′ exon element comprises a 3′ internal spacer.

In some embodiments, the circular RNA polynucleotide comprises a 5′ internal duplex and a 3′ internal duplex. See, e.g., supra.

In some embodiments, the circular RNA polynucleotide comprises a 5′ internal homology region and/or a 3′ internal homology region. See, e.g., supra.

In some embodiments, the circular RNA polynucleotide comprises internal spacers (IS) of different lengths, e.g., a 5′internal spacer and/or a 3′ internal spacer. See, e.g., supra.

In some embodiments, the circular RNA polynucleotide retains portions of the precursor RNA polynucleotides, described elsewhere herein in detail. In some embodiments, portions of the precursor RNA polynucleotide are removed upon circularization. For example, in some embodiments, the circular RNA polynucleotide does not comprise a 5′ external spacer and/or a 3′ external spacer. In some embodiments, the circular RNA polynucleotide does not comprise a 5′ intron segment and/or 3′ intron segment. In some embodiments, the circular RNA polynucleotide does not comprise affinity tags. In some embodiments, the circular RNA polynucleotide does not comprise external homology regions. In some embodiments, the circular RNA polynucleotide does not retain a portion of a monotron element. In certain embodiments, the circular RNA polynucleotide does not retain a monotron element.

In some embodiments, and as described in more detail elsewhere herein, the circular RNA polynucleotide comprises modified nucleotides and/or modified nucleosides, namely comprising at least one modified A, C, G, or U/T nucleotide or nucleoside. Exemplary modifications are described in detail elsewhere herein. See, e.g., infra. In some embodiments, a circular RNA polynucleotide comprises modified nucleotides and/or modified nucleosides where between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. In some embodiments, portions of the polynucleotide comprise between 1% and 10% modification of the nucleotides or nucleosides. In some embodiments, portions of the circular RNA polynucleotide comprise less than 10% modification. In some embodiments, portions of the polynucleotide or the polynucleotide in its entirety comprise no nucleotide or nucleoside modifications. In some embodiments, a circular RNA polynucleotide may lack modifications, where the linear precursors used to produce the circular RNA polynucleotide contained modifications (e.g., in the introns). In some embodiments, incorporation of a nucleotide or nucleoside modification to a precursor RNA polynucleotide hinders or lowers the capacity of the circular RNA to circularize, splice, or express. In some embodiments, the circular RNA polynucleotide is from about 50 nucleotides to about 15 kilobases in length.

In some embodiments, the circular RNA polynucleotide has an in vivo duration of therapeutic effect in a subject of at least about 10 hours. In some embodiments, the circular RNA polynucleotide has a functional half-life of at least about 10 hours. In some embodiments, the circular RNA polynucleotide has a duration of therapeutic effect in a cell greater than or equal to that of a linear mRNA polynucleotide comprising the same expression sequence. In some embodiments, the circular RNA polynucleotide has a functional half-life in a cell greater than or equal to that of a linear mRNA polynucleotide comprising the same expression sequence. In some embodiments, the circular RNA polynucleotide has an in vivo duration of therapeutic effect in a subject greater than that of a linear mRNA polynucleotide having the same expression sequence. In some embodiments, the circular RNA polynucleotide has an in vivo functional half-life in a subject greater than that of a linear mRNA polynucleotide having the same expression sequence.

In some embodiments, provided herein is a non-naturally occurring RNA polynucleotide comprising a translation initiation element (TIE), an expression sequence encoding a CD19 binding molecule (e.g., with which the TIE is not naturally associated), and a means for self-splicing. In some embodiments, provided herein is a non-naturally occurring RNA polynucleotide comprising a translation initiation element (TIE), an expression sequence encoding a CD19 binding molecule (e.g., with which the TIE is not naturally associated), and a means for self-circularization. In some embodiments, provided herein is provided herein is a non-naturally occurring RNA polynucleotide comprising a translation initiation element (TIE), an expression sequence encoding a CD19 binding molecule (e.g., with which the TIE is not naturally associated), and an autocatalytic intron-exon means for self-splicing. In some embodiments, provided herein is a non-naturally occurring RNA polynucleotide comprising a translation initiation element (TIE), an expression sequence encoding a CD19 binding molecule (e.g., with which the TIE is not naturally associated), and an autocatalytic intron-exon means for self-circularization. In some embodiments, provided herein is a non-naturally occurring RNA polynucleotide comprising, in the following order, a 3′ exon segment means for self-splicing, a translation initiation element, an expression sequence encoding a CD19 binding molecule (e.g., with which the TIE is not naturally associated), and a 5′ exon segment means for self-splicing. In some embodiments, provided herein is a non-naturally occurring RNA polynucleotide comprising, in the following order, a 3′ exon segment means for self-circularization, a TIE, an expression sequence encoding a CD19 binding molecule (e.g., with which the TIE is not naturally associated), and a 5′ exon segment means for self-circularization. In some embodiments, provided herein is a non-naturally occurring RNA polynucleotide comprising, in the following order, a 3′ exon segment, a TIE, an expression sequence encoding a CD19 binding molecule, and a 5′ exon segment, wherein the exon segments are means for self-splicing. In some embodiments, provided herein is a non-naturally occurring RNA polynucleotide comprising, in the following order, a 3′ exon segment, a TIE, an expression sequence encoding a CD19 binding molecule, and a 5′ exon segment, wherein the exon segments are means for self-circularization. In some embodiments, provided herein is a circular RNA polynucleotide comprising, in the following order, a 3′ exon segment means for self-circularization, a TIE, an expression sequence encoding a CD19 binding molecule, and a 5′ exon segment means for self-circularization. In some embodiments, provided herein is a circular RNA polynucleotide comprising, in the following order, a 3′ exon segment, a TIE, an expression sequence encoding a CD19 binding molecule, and a 5′ exon segment, wherein the exon segments are means for self-splicing.

### A. Precursor Linear RNA

Disclosed herein are precursor RNAs capable of producing circular RNAs. In some embodiments, the precursor RNA is a linear RNA that comprises both 5′ intron and exon elements and 3′ exon and intron elements or comprising only 3′ exon and intron elements for producing circular RNAs with enhanced circularization efficiency.

Accordingly, provided herein is a precursor RNA polynucleotide capable of producing a circular RNA polynucleotide after splicing, wherein the precursor RNA polynucleotide comprises both 5′ intron and exon elements and 3′ exon and intron elements (e.g., combined accessory elements). Also provided is a precursor RNA polynucleotide capable of producing a circular RNA polynucleotide after splicing, wherein the precursor RNA polynucleotide comprises only 3′ exon and intron elements.

In some embodiments, a provided precursor RNA polynucleotide comprises (i) 3′ permuted intron segment comprising a 5′ nucleotide of a 3′ splice site dinucleotide; and (ii) a 3′ exon segment comprising a 3′ nucleotide of a 3′ splice site dinucleotide. Exemplary splice site dinucleotides are provided in the Table set forth herein. In some embodiments, a provided precursor RNA polynucleotide comprises (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, a provided precursor RNA polynucleotide comprises a terminal element comprising (a) an excised terminal segment and a retained terminal segment or (b) a natural exon or a fragment thereof.

In some embodiments, a provided precursor RNA polynucleotide comprises (i) a 5′ intron element comprising a 3′ permuted intron segment comprising a 5′ nucleotide of a 3′ splice site dinucleotide; (ii) a 5′ exon element comprising a 3′ exon segment comprising a 3′ nucleotide of a 3′ splice site dinucleotide; (iii) a 3′ exon element comprising a 5′ exon segment comprising a 5′ nucleotide of a 5′ splice site; and (iv) a 3′ intron element comprising a 5′ permuted intron segment comprising a 3′ nucleotide of a 5′ splice site dinucleotide. In some embodiments, a provided precursor RNA polynucleotide comprises (i) a terminal element comprising (a) an excised terminal segment and/or a retained terminal segment or (b) a natural exon or a fragment thereof; (ii) a 5′ intron element comprising a 3′ permuted intron segment comprising a 5′ nucleotide of a 3′ splice site dinucleotide; and (iii) a 5′ exon element comprising a 3′ exon segment comprising a 3′ nucleotide of a 3′ splice site dinucleotide.

In some embodiments, a provided precursor RNA polynucleotide comprises a 5′ combined accessory element comprising (i) a 3′ permuted intron segment comprising a 5′ nucleotide of a 3′ splice site dinucleotide; and (ii) a 3′ exon segment comprising a 3′ nucleotide of a 3′ splice site dinucleotide. In some embodiments, element (ii) is located upstream to the intervening region. In some embodiments, the 5′ combined accessory element comprises a 3′ exon segment comprising a Group I or Group II exon 3′ nucleotide of a 3′ splice site dinucleotide.

In some embodiments, a provided precursor RNA polynucleotide comprises a 3′ combined accessory element comprising (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, element (ii) is located downstream to the intervening region. In some embodiments, a 3′ combined accessory element comprises a 5′ exon segment comprising a Group I or Group II exon 5′ nucleotide of a 5′ splice site dinucleotide.

In some embodiments, a provided precursor RNA polynucleotide comprises a 5′ combined accessory element, an intervening region, and a 3′ combined accessory element. In some embodiments, (a) the 5′ combined accessory element comprises (i) a 3′ permuted intron segment comprising a 5′ nucleotide of a 3′ splice site dinucleotide; and (ii) a 3′ exon segment comprising a 3′ nucleotide of a 3′ splice site dinucleotide; and (b) the 3′ combined accessory element comprising (i) a 5′ exon segment comprising a 5′ nucleotide of a 5′ splice site dinucleotide; and (ii) a 5′ permuted intron segment comprising a 3′ nucleotide of a 5′ splice site dinucleotide. In some embodiments, the 5′ nucleotide of a 3′ splice site dinucleotide, 3′ nucleotide of a 3′ splice site dinucleotide, 5′ nucleotide of a 5′ splice site dinucleotide and 3′ nucleotide of a 5′ splice site dinucleotide are optionally a combination of nucleotides or a portion of a sequence selected from PCT/US2024/027627.

In some embodiments, the 5′ combined accessory element is located 5′ to the intervening region; and the intervening region is located is 5′ to the 3′ combined accessory element.

In some embodiments, a provided precursor RNA polynucleotide comprises a terminal element, an intervening region (e.g., comprising an expression sequence encoding a CD19 binding molecule), and a monotron element. In some embodiments, the monotron element is located 5′ to the intervening region, which is located 5′ to the terminal element. In other embodiments, the monotron element is located 3′ to the intervening region, which is located 3′ to the terminal element. As set forth in further detail below, in some embodiments, the terminal element comprises a splice site nucleotide and the monotron element comprises a splice site dinucleotide and a splice site nucleotide.

In some embodiments, the precursor RNA polynucleotide is linear.

In some embodiments, permuted intron-exon splicing results in circularization of the precursor RNA polynucleotide. During splicing, a transesterification reaction can occur at the 5′ splice site and a second transesterification reaction can occur at the 3′ splice site. In some embodiments, splicing of the precursor RNA polynucleotide results in the removal of the 3′ intron element and the 5′ intron element. Accordingly, the circular RNA polynucleotide produced after splicing of the precursor RNA polynucleotide lacks the 3′ intron segment and the 5′ intron segment, but retains the 3′ exon segment and the 5′ exon segment.

In some embodiments, the precursor RNA polynucleotide is capable of circularizing when incubated in the presence of one or more guanosine nucleotides or nucleoside (e.g., GTP) and a divalent cation (e.g., Mg²⁺).

In some embodiments, the precursor RNA polynucleotide is between 300 and 10000, between 400 and 9000, between 500 and 8000, between 600 and 7000, between 700 and 6000, between 800 and 5000, between 900 and 5000, between 1000 and 5000, between 1100 and 5000, between 1200 and 5000, between 1300 and 5000, between 1400 and 5000, or between 1500 and 5000 nucleotides (nt) in length. In some embodiments, the precursor RNA polynucleotide is at least 300 nt, at least 400 nt, at least 500 nt, at least 600 nt, at least 700 nt, at least 800 nt, at least 900 nt, at least 1000 nt, at least 1100 nt, at least 1200 nt, at least 1300 nt, at least 1400 nt, at least 1500 nt, at least 2000 nt, at least 2500 nt, at least 3000 nt, at least 3500 nt, at least 4000 nt, at least 4500 nt, or at least 5000 nt in length. In some embodiments, the precursor RNA polynucleotide is no more than 3000 nt, no more than 3500 nt, no more than 4000 nt, no more than 4500 nt, no more than 5000 nt, no more than 6000 nt, no more than 7000 nt, no more than 8000 nt, no more than 9000 nt, or no more than 10000 nt in length. In some embodiments, the precursor RNA polynucleotide is about 300 nt, about 400 nt, about 500 nt, about 600 nt, about 700 nt, about 800 nt, about 900 nt, about 1000 nt, about 1100 nt, about 1200 nt, about 1300 nt, about 1400 nt, about 1500 nt, about 2000 nt, about 2500 nt, about 3000 nt, about 3500 nt, about 4000 nt, about 4500 nt, about 5000 nt, about 6000 nt, about 7000 nt, about 8000 nt, about 9000 nt, or about 10000 nt in length.

In various embodiments, provided herein are DNA templates that transcribe into precursor RNA polynucleotides of the disclosure. Accordingly, provided herein are DNA templates comprising sequences encoding the precursor RNAs of the disclosure. In some embodiments, the DNA template or polynucleotide of the present disclosure comprises a vector, a PCR product, a plasmid, a minicircle DNA, a cosmid, an artificial chromosome, a complementary DNA (cDNA), an extrachromosomal DNA (ecDNA), a doggybone DNA (dbDNA), a close-ended DNA (ceDNA), a viral polynucleotide, or a fragment thereof. In some embodiments, the polynucleotide of the present disclosure is selected from a DNA plasmid, a cosmid, a PCR product, dbDNA, close-ended DNA (ceDNA), and a viral polynucleotide. In some embodiments, the polynucleotide further comprises a promoter segment or sequence. In some embodiments, the DNA template is linearized. In other embodiments, the DNA template is non-linearized. In some embodiments, the DNA template is single-stranded. In some embodiments, the DNA template is double-stranded. In some embodiments, the DNA template comprises in whole or in part from a viral, bacterial or eukaryotic vector.

In various embodiments, provided herein is a circular RNA polynucleotide produced by circularization of a precursor RNA polynucleotide described herein.

In some embodiments, the circular RNA polynucleotide is produced inside a cell. In some embodiments, a provided precursor RNA is transcribed using a DNA template in the cytoplasm (e.g., by a bacteriophage RNA polymerase) or nucleus (e.g., by host RNA polymerase II) and then circularized.

In some embodiments, the circular RNA polynucleotide is between 300 and 10000, between 400 and 9000, between 500 and 8000, between 600 and 7000, between 700 and 6000, between 800 and 5000, between 900 and 5000, between 1000 and 5000, between 1100 and 5000, between 1200 and 5000, between 1300 and 5000, between 1400 and 5000, or between 1500 and 5000 nucleotides (nt) in length. In some embodiments, the circular RNA polynucleotide is at least 300 nt, at least 400 nt, at least 500 nt, at least 600 nt, at least 700 nt, at least 800 nt, at least 900 nt, at least 1000 nt, at least 1100 nt, at least 1200 nt, at least 1300 nt, at least 1400 nt, at least 1500 nt, at least 2000 nt, at least 2500 nt, at least 3000 nt, at least 3500 nt, at least 4000 nt, at least 4500 nt, or at least 5000 nt in length. In some embodiments, the circular RNA polynucleotide is no more than 3000 nt, no more than 3500 nt, no more than 4000 nt, no more than 4500 nt, no more than 5000 nt, no more than 6000 nt, no more than 7000 nt, no more than 8000 nt, no more than 9000 nt, or no more than 10000 nt in length. In some embodiments, the circular RNA polynucleotide is about 300 nt, about 400 nt, about 500 nt, about 600 nt, about 700 nt, about 800 nt, about 900 nt, about 1000 nt, about 1100 nt, about 1200 nt, about 1300 nt, about 1400 nt, about 1500 nt, about 2000 nt, about 2500 nt, about 3000 nt, about 3500 nt, about 4000 nt, about 4500 nt, about 5000 nt, about 6000 nt, about 7000 nt, about 8000 nt, about 9000 nt, or about 10000 nt in length.

Circular RNA polynucleotides lack the free ends necessary for exonuclease-mediated degradation, causing them to be resistant to several mechanisms of RNA degradation and granting extended half-lives when compared to linear mRNA comprising the same expression sequence. Circularization may allow for the stabilization of RNA polynucleotides that generally suffer from short half-lives and may improve the overall efficacy of exogenous mRNA in a variety of applications.

In some embodiments, the circular RNA polynucleotide has a functional half-life of at least 5 hours, 10 hours, 15 hours, 20 hours, 30 hours, 40 hours, 50 hours, 60 hours, 70 hours, or 80 hours. In some embodiments, the circular RNA polynucleotide has a functional half-life of 5-80, 10-70, 15-60, or 20-50 hours. In some embodiments, the circular RNA polynucleotide has a functional half-life greater (e.g., at least 1.5-fold greater or at least 2-fold greater) than that of a linear mRNA polynucleotide comprising the same expression sequence. In some embodiments, the circular RNA polynucleotide, or a pharmaceutical composition thereof, has a functional half-life in a human cell greater than or equal to that of a pre-determined threshold value. In some embodiments, the functional half-life is determined by a functional protein assay. For example, in some embodiments, the functional half-life is determined by an in vitro luciferase assay, wherein the activity of Gaussia luciferase (GLuc) is measured in the media of human cells (e.g., HepG2) expressing the circular RNA polynucleotide every 1, 2, 6, 12, or 24 hours over 1, 2, 3, 4, 5, 6, 7, or 14 days. In some embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear mRNA polynucleotide comprising the same expression sequence as the circular RNA polynucleotide. In some embodiment, the functional half-life of a circular RNA polynucleotides provided herein in eukaryotic cells (e.g., mammalian cells, such as human cells) as assessed by protein synthesis is at least 20 hours (e.g., at least 80 hours).

In some embodiments, the circular RNA polynucleotide provided herein has higher functional stability than a linear mRNA comprising the same expression sequence. In some embodiments, the circular RNA provided herein has higher functional stability than a linear mRNA comprising the same expression sequence, 5moU modifications, optimized UTR, cap, and/or polyA tail. In some embodiments, the circular RNA provided herein has greater durability than a linear mRNA comprising the same expression sequence.

In some embodiments, a provided circular RNA polynucleotide may have a higher magnitude of expression, e.g., a higher magnitude of expression 24 hours after administration of RNA to cells, than a linear mRNA comprising the same expression sequence. In some embodiments, the circular RNA polynucleotide has a higher magnitude of expression than an mRNA comprising the same expression sequence, 5moU modifications, optimized UTR, cap, and/or polyA tail. In some embodiments, the circular RNA provided herein has a greater duration of expression than that of a linear mRNA polynucleotide comprising the same expression sequence. In some embodiments, the circular RNA provided herein has a greater duration of expression of a CAR (e.g., CD19 CAR) than that of a linear mRNA polynucleotide comprising the same expression sequence. In some embodiments, the circular RNA provided herein has a greater duration of expression of a CAR (e.g., CD19 CAR) in immune cells (e.g., T cells) than that of a linear mRNA polynucleotide comprising the same expression sequence.

In some embodiments, a provided circular RNA polynucleotide is transfected into a cell. In some embodiments, the DNA template, which transcribes into the precursor RNA polynucleotide from which the circular RNA polynucleotide is produced, is transfected into a cell and subsequently transcribed in the cell. Transcription of the circular RNA from the transfected DNA template may be induced via polymerases. In some embodiments, the polymerases are endogenous polymerases of the cell. In some embodiments, the polymerases are added to the cell. In some other embodiments, the polymerases are encoded by one or more nucleic acids transfected into the cell.

In some embodiments, the circular RNA polynucleotide is administered to an animal (e.g., a human) such that a CD19 binding molecule, e.g., CD19 CAR, encoded by the circular RNA polynucleotide is expressed inside the animal.

In some embodiments, a provided circular RNA is less immunogenic than a linear mRNA comprising the same expression sequence when exposed to an immune system of an organism or a certain type of immune cell. In some embodiments, the circular RNA is associated with modulated production of cytokines when exposed to an immune system of an organism or a certain type of immune cell. For example, in some embodiments, the circular RNA is associated with reduced production of IFN-β1, RIG-I, IL-2, IL-6, IFNγ, and/or TNFα when exposed to an immune system of an organism or a certain type of immune cell as compared to linear mRNA comprising the same expression sequence. In some embodiments, the circular RNA is associated with less IFN-β1, RIG-I, IL-2, IL-6, IFNγ, and/or TNFα transcript induction when exposed to an immune system of an organism or a certain type of immune cell as compared to linear mRNA comprising the same expression sequence. In some embodiments, the circular RNA is less immunogenic than linear mRNA comprising the same expression sequence. In some embodiments, the circular RNA is less immunogenic than linear mRNA comprising the same expression sequence, 5moU modifications, an optimized UTR, a cap, and/or a polyA tail.

In some embodiments, the circular RNA provided herein has greater functionality, and/or exhibits greater cytotoxicity levels in an immune cells, and/or exhibits greater B cell depletion when expressed, and/or exhibits greater B cell depletion over time and/or over a longer period of time when expressed, and/or exhibits greater B cell depletion in the blood (e.g., peripheral blood), spleen, bone marrow and/or lymph nodes (e.g., mesenteric lymph nodes and/or pooled draining lymph nodes) when expressed as compared to linear mRNA comprising the same expression sequence. In some embodiments, the circular RNA encoding a CD19 binding molecule (e.g., CD19 CAR) has greater functionality than linear mRNA comprising the same expression sequence. In some embodiments, the circular RNA encoding a CD19 binding molecule (e.g., CD19 CAR) has greater cytotoxicity levels in an immune cell compared to linear mRNA comprising the same expression sequence. In some embodiments, the circular RNA encoding a CD19 binding molecule (e.g., CD19 CAR) comprises greater B cell depletion when expressed compared to linear mRNA comprising the same expression sequence. In some embodiments, the circular RNA encoding a CD19 binding molecule (e.g., CD19 CAR) comprises greater B cell depletion over time and/or over a longer period of time when expressed compared to linear mRNA comprising the same expression sequence. In some embodiments, the circular RNA encoding a CD19 binding molecule (e.g., CD19 CAR) comprises greater B cell depletion in the blood (e.g., peripheral blood), spleen, bone marrow and/or lymph nodes (e.g., mesenteric lymph nodes and/or pooled draining lymph nodes) when expressed compared to linear mRNA comprising the same expression sequence. In some embodiments herein, a subject's B cell count is decreased 24 hours or 48 hours post-treatment. In some embodiments, a subject's B cell count is decreased at, e.g., 24 hours, 48 hours, 3 days, 4 days, 5 days, 6 days, 7 days, 8 days, or 9 days post-treatment, as compared to a baseline value (e.g., pre-treatment). In some embodiments, a subject's B cell count continues to decrease and/or maintains a decreased value (e.g., compared to baseline) beyond 10 days post-treatment.

Various circular RNA, circular RNA constructs, compositions comprising circular RNA, precursor RNA, and related methods are described, for example in U.S. patent application Ser. No. 17/853,576, WO2019236673, WO2020237227, WO2021113777, WO2021226597, WO2021189059, WO2021236855, WO2022261490, WO2023056033, WO2023081526, WO2023141586, WO2023250375, and WO2024102677, which are each incorporated by reference in their entireties.

### B. Intron Elements, Exon Elements, Terminal Elements

Polynucleotides provided herein (e.g., DNA templates, precursor RNA polynucleotides, or circular RNA polynucleotides) may comprise one or more intron elements, exon elements, and/or terminal elements. In some embodiments, each intron element, exon element, and terminal element may independently comprise one or more spacers, intron segments, exon segments, duplex regions, affinity sequences, and/or untranslated elements. These sequence elements within the intron elements, exon elements, or terminal elements are arranged to optimize circularization and/or protein expression.

a. Intron and Exon Elements

In various embodiments, an intron element (e.g., 3′ intron element or 5′ intron element) comprises a permuted intron segment. In some embodiments, a 3′ permuted intron segment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural intron (e.g., a group I or group II intron) including the 5′ nucleotide of the 3′ splice site dinucleotide. In some embodiments, a 5′ permuted intron fragment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural intron (e.g., a group I or group II intron) including the 3′ nucleotide of the 5′ splice site dinucleotide. Exemplary splice site dinucleotides are described in the Table herein.

In some embodiments, an intron element comprises an intron derived from a trans-splicing ribozyme. In some embodiments, the intron element comprises a Group I trans-splicing ribozyme (e.g., a Tetrahymena trans-splicing ribozyme) segment. In some embodiments, the trans-splicing ribozyme segment along with an exon segment that may cleave a target site (e.g., a sequence of interest and/or a coding element) and subsequently ligate cleaved targe site to a 3′ exon to form a circular RNA product.

In various embodiments, a provided polynucleotide (e.g., a DNA template or a precursor RNA polynucleotide) comprises a 5′ exon element located upstream to the intervening region. In some embodiments, a provided polynucleotide comprises a 3′ intron element located downstream to the intervening region. In various embodiments, a provided polynucleotide comprises a 3′ exon element located upstream to the intervening region. In some embodiments, a provided polynucleotide comprises a 3′ intron element located upstream to the intervening region.

According to the present disclosure, the 3′ exon element and 5′ exon element each comprise an exon segment. In some embodiments, the 5′ exon element comprises a 3′ exon segment. In some embodiments, the 3′ exon element comprises a 5′ exon segment. In some embodiments, the 3′ and/or 5′ exon segment is a self-spliced or self-splicing exon segment. In some embodiments, the self-spliced and/or self-splicing exon segment comprises in part or in whole a naturally occurring exon sequence from a virus, bacterium or eukaryotic DNA vector. In other embodiments, the self-spliced and/or self-splicing exon segment comprises in part or in whole a non-naturally occurring sequence.

In some embodiments, a 3′ exon segment is a contiguous sequence at least 75% homologous (e.g., at least 80%, 85%, 90%, 91%, 92%, 93%, 94%, 95%, 96%, 97%, 98%, 99% or 100% homologous) to the 5′-proximal end of an exon adjacent a 3′ intron segment as described herein, including the 3′ nucleotide of the splice site dinucleotide. In some embodiments, a 5′ exon segment is a contiguous sequence at least 75% homologous (e.g., at least 80%, 85%, 90%, 91%, 92%, 93%, 94%, 95%, 96%, 97%, 98%, 99% or 100% homologous) to the 3′-proximal end of an exon adjacent a 5′ intron segment as described herein, including the 5′ nucleotide of the splice site dinucleotide.

In some embodiments, at least one of the exon segments is less than 15 nucleotides in length. In some embodiments, the 3′ exon segment and/or 5′ exon segment comprises a Group I exon segment or a Group II exon segment less than 15 nucleotides in length.

In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some embodiments, the circular RNA comprises a self-spliced exon segment that is 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides from the exonic sequences of Table 2 (in which sequences are shown as 15-nucleotide exonic sequence, intronic sequence, 15-nucleotide exonic sequence), e.g., contiguous nucleotides from the 5′ or 3′ end of the exonic sequences of Table 2. In some embodiments, the circular RNA comprises a self-spliced exon segment that is 5 nucleotides, 6 nucleotides, 7 nucleotides, 8 nucleotides, 9 nucleotides, or 10 nucleotides from the exonic sequences of Table 3 (in which sequences are shown as 10-nucleotide exonic sequence, intronic sequence, 10-nucleotide exonic sequence), e.g., contiguous nucleotides from the 5′ or 3′ end of the exonic sequences of Table 3. See also PCT/US2024/027627.

In some embodiments, the intron segment is a Group I intron and the exon segment comprises a Group I self-splicing exon segment. In some embodiments, the intron segment is a Group II intron and the exon segment comprises a Group II self-splicing exon segment.

In some embodiments, the exon element comprises a sequence directed to a native Group I intron-adjacent exon segment sequence or Group II intron-adjacent exon segment sequence, or fragment thereof. In some embodiments, the exon element comprises at least one mutation of a native Group I intron-adjacent exon segment sequence or Group II intron-adjacent exon segment sequence, or fragment thereof. In some embodiments, the exon element comprises at least one deletion of a native Group I intron-adjacent exon segment sequence or Group II intron-adjacent exon segment sequence, or fragment thereof. In some embodiments, the exon element comprises at least one insertion of a native Group I intron-adjacent exon segment sequence or Group II intron-adjacent exon segment sequence, or fragment thereof. In some embodiments, the native Group I intron segment or Group II intron segment sequences are selected from a sequence in Table 2 or Table 3, below.

b. Terminal Elements

In various embodiments, a provided polynucleotide (e.g., a DNA template or a precursor RNA polynucleotide) comprises a terminal element. In some embodiments, the terminal element is located upstream to the intervening region. In some embodiments, the terminal element is non-intronic. In some embodiments, the terminal element lacks one or both nucleotides of a natural splice site dinucleotide associated with a natural Group I or Group II intron sequence. In some embodiments, a portion or the entire terminal element is excised after circularization of a precursor RNA polynucleotide comprising said terminal element.

In some embodiments, a polynucleotide comprises a terminal element, an intervening region, and a monotron. In some embodiments, the polynucleotide comprises, in the following order, a terminal element, an intervening region, and a monotron. In some embodiments, the polynucleotide comprises, in the following order, a monotron, an intervening region, and a terminal element. In some embodiments, the terminal element comprises a splice site nucleotide capable of engaging in a transesterification reaction with the monotron.

In some embodiments, the terminal element comprises an excised terminal segment and a retained terminal segment. In the same embodiments, the retained terminal segment is retained after circularization of a precursor RNA polynucleotide comprising such a terminal element. In the same embodiments, the exercised terminal segment is not retained after circularization of a precursor RNA polynucleotide comprising such a terminal element. In still the same embodiments, the nucleotide sequence of the terminal element is non-natural or synthetic.

In some embodiments, the terminal element comprises a natural exon or a fragment thereof. In some embodiments, the terminal element is retained after circularization of a precursor RNA polynucleotide comprising said terminal element.

In some embodiments, the terminal element is capable of binding to a 3′ intron element (e.g., the 3′ intron element comprised in the same polynucleotide). In some embodiments, the terminal element is capable of directing or functionalizing the splicing activity of a 3′ intron element (e.g., the 3′ intron element comprised in the same polynucleotide).

c. Monotron

Provided herein is a precursor RNA polynucleotide comprising a monotron (also called a monotron element or monotron sequence) and a terminal element (also called a terminal sequence). In some embodiments, the monotron has ribozymatic activity that allows it to enzymatically self-cleave. In some embodiments, the monotron is capable of forming a phosphodiester bond with a terminal sequence, i.e., a sequence containing a splice site dinucleotide and optionally a natural exon sequence or fragment thereof. In some embodiments, the precursor RNA polynucleotide comprises a terminal element; an intervening region, and a monotron element.

In some embodiments, the precursor RNA polynucleotide comprises, in the following order, (a) a terminal element; (b) an intervening region (e.g., comprising an expression sequence encoding a CD19 binding molecule, e.g., CD19 CAR), and (c) a monotron element. In some embodiments, the terminal sequence is upstream of the monotron sequence in the precursor RNA polynucleotide. In such embodiments: (i) the terminal element comprises a splice site nucleotide, (ii) the monotron element comprises a splice site dinucleotide at or near the 5′ end of the monotron, and (iii) the monotron element is capable of interacting with a nucleophile that is capable of cleaving at the splice site dinucleotide at or near the 5′ end of the monotron, where the cleavage product of (iii) comprises a 5′ splice site nucleotide that is capable of cleaving at the splice site nucleotide of the terminal element. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site dinucleotide at or near the 5′ end of the monotron. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments, the precursor RNA polynucleotide comprises, in the following order, (a) a monotron element; (b) an intervening region (e.g., comprising an expression sequence encoding a CD19 binding molecule, e.g., CD19 CAR), and (c) terminal element. In some embodiments, the monotron sequence is upstream of the terminal sequence in the precursor RNA polynucleotide. In such embodiments: (i) the monotron element comprises a splice site dinucleotide at or near the 3′ end of the monotron, (ii) the terminal element comprises a splice site nucleotide, and (iii) the monotron element is capable of interacting with a nucleophile that is capable of cleaving at the splice site nucleotide of the terminal element, where the cleavage product of (iii) comprises a 5′ splice site nucleotide that is capable of cleaving at the splice site dinucleotide at or near the 3′ end of the monotron. In some embodiments, the nucleophile is a free nucleophile that is introduced to the precursor RNA polynucleotide, e.g., not in cis and/or covalently linked to the precursor RNA polynucleotide. In some embodiments, the nucleophile is a guanosine that is capable of cleaving at the splice site nucleotide of the terminal element. In some embodiments, the guanosine is a free guanosine that is introduced to the precursor RNA polynucleotide, e.g., not in cis and/or covalently linked to the precursor RNA polynucleotide. In some embodiments, the cleavage product of (iii) comprises a 5′ splice site nucleotide having a 3′ hydroxyl group that is capable of cleaving at the splice site nucleotide of the terminal element.

In some embodiments where the terminal sequence is upstream to the monotron, the monotron can perform two transesterification reactions. The monotron can (a) self-cleave and (b) form a phosphodiester bond with the terminal sequence. In some embodiments, the reactions (a) and (b) are sequential. In some embodiments, (a) the monotron is capable of interacting with a nucleophile that is capable of cleaving at the splice site dinucleotide at or near the 5′ end of the monotron, and (b) the cleavage product of (a), i.e., the 5′ splice site nucleotide, e.g., having a 3′ hydroxyl group, engages in a transesterification reaction (cleaves) at the splice site nucleotide of the terminal sequence, yielding a circular RNA or oRNA. In these embodiments, the monotron interacts with the nucleophile by forming a binding pocket with the nucleophile, and the linear precursor is capable of adopting a conformation in which the nucleophile is in proximity to and is capable of cleaving at the splice site dinucleotide at or near the 5′ end of the monotron. In some embodiments, the nucleophile can be a guanosine, e.g., a free guanosine that is introduced to the precursor RNA polynucleotide.

In some embodiments where the monotron sequence is upstream of the terminal sequence, the monotron can also perform two transesterification reactions. In some embodiments, (a) the monotron is capable of interacting with a nucleophile that is capable of cleaving at the splice site nucleotide of the terminal element, and (b) the cleavage product of (a), i.e., the 5′ splice site nucleotide, e.g., having a 3′ hydroxyl group, engages in a transesterification reaction (cleaves) at the splice site dinucleotide at or near the 3′ end of the monotron, yielding a circular RNA or oRNA. In these embodiments, the monotron interacts with the nucleophile by forming a binding pocket with the nucleophile, and the linear precursor is capable of adopting a conformation in which the nucleophile is in proximity to and is capable of cleaving the splice site nucleotide of the terminal element. In some embodiments, the nucleophile can be a guanosine, e.g., a free guanosine that is introduced to the precursor RNA polynucleotide.

In some embodiments, the monotron comprises a 5′ proximal end of a natural group I or group II intron including the splice site dinucleotide and optionally a natural exon sequence or fragment thereof. In some embodiments, the 5′ end of the monotron refers to nucleotides within the 5′ half of the monotron. In some embodiments, the 3′ end of the monotron refers to nucleotides within the 3′ half of the monotron. In some embodiments, at or near the 5′ end of the monotron refers to within the 5′ half of the monotron. In some embodiments, at or near the 5′ end of the monotron refers to within the first ten 5′ positions in the monotron. In some embodiments, at the 5′ end of the monotron refers to the first 5′ position(s) in the monotron. In some embodiments, at or near the 3′ end of the monotron refers to within the 3′ half of the monotron. In some embodiments, at or near the 3′ end of the monotron refers to within the last ten 3′ positions in the monotron. In some embodiments, at the 3′ end of the monotron refers to last 3′ position(s) in the monotron.

In some embodiments, the splice site nucleotide of the terminal element is not a natural splice site dinucleotide associated with a natural Group I or Group II intron sequence. In some embodiments, the terminal element comprises at least a portion of a natural exon or a fragment of a natural exon. In some embodiments, the natural exon is a Group I or Group II exon. In some embodiments, the natural exon or fragment thereof is 10-20 nucleotides in length. In some embodiments, the terminal element comprises a synthetic derivative of a natural exon or fragment thereof. In some embodiments, the terminal element comprises an exon or synthetic nucleotides that are longer than the splice site nucleotide that can help with splicing.

In some embodiments, the terminal element sequence has a percent sequence identity of about 70%, 75%, 80%, 85%, 90%, 91%, 92%, 93%, 94%, 95%, 96%, 97%, 98%, 99% or more to an exon fragment of a sequence selected from Table 2 or Table 3. In some embodiments, the terminal element sequence comprises an exon fragment comprising one, two, three, four, five, six, seven, eight, nine, ten, or more mutations to a sequence selected from Table 2 or Table 3. The mutations are, for example, selected from insertions, deletions, mutations, additions, and subtractions. In some embodiments, the terminal element or exon fragment thereof comprises a polynucleotide sequence selected from a sequence set forth in PCT/US2024/027627.

In some embodiments, the terminal element is less than 500, less than 450, less than 400, less than 350, less than 300, less than 250, less than 200, less than 150, less than 100, less than 90, less than 80, less than 70, less than 60, less than 50, less than 40, less than 30, less than 20, or less than 10 nucleotides in length.

In some embodiments, the terminal element is capable of directing or functionalizing the splicing activity of the monotron element.

In some embodiments, a portion of the terminal segment is retained upon circularization. In some embodiments, a portion of the terminal segment is excised upon circularization. In some embodiments, all or a portion of the terminal element is excised post-circularization. In some embodiments, the terminal element is not excised upon cleavage and is retained post-cleavage.

In some embodiments, the monotron element comprises at least a portion of a Group I or Group II intron. In some embodiments, Group I or Group II intron is selected from a genus and/or species described in Table 2 or Table 3. In some embodiments, the Group I or Group II intron is from a gene selected from Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, and Tilletiopsis orzyzicola. In some embodiments, the monotron element or a fragment thereof has a percent sequence identity of about 70%, 75%, 80%, 85%, 90%, 91%, 92%, 93%, 94%, 95%, 96%, 97%, 98%, 99% or more to a sequence selected from Table 2 or Table 3. In some embodiments, the monotron element sequence or fragment thereof comprises one, two, three, four, five, six, seven, eight, nine, ten, or more mutations to a sequence selected from Table 2 or Table 3. The mutations are, for example, selected from insertions, deletions, additions, and subtractions. In some embodiments the monotron element sequence or fragment thereof comprises a polynucleotide sequence having a percent sequence identity of about 70%, 75%, 80%, 85%, 90%, 91%, 92%, 93%, 94%, 95%, 96%, 97%, 98%, 99%, or more to a portion of a sequence set forth in PCT/US2024/027627.

In some embodiments, the Group I or Group II intron or introns, or portion thereof, are at least 10 nucleotides in length.

In some embodiments, the monotron element comprises at least one mutation of a native Group I intron-adjacent exon sequence or Group II intron-adjacent exon sequence. In some embodiments, the at least one mutation is at least one substation mutation of a native Group I intron-adjacent exon sequence or Group II intron-adjacent exon sequence. In some embodiments, the at least one mutation is at least one deletion of a native Group I intron-adjacent exon sequence or Group II intron-adjacent exon sequence. In some embodiments, at least one of the exon segments is less than 15 nucleotides in length. In some embodiments, the monotron element comprises a 3′ exon segment and/or 5′ exon segment, wherein the 3′ or 5′ exon segment comprises a Group I exon segment or a Group II exon segment less than 15 nucleotides in length. In some embodiments, the 3′ exon segments and/or 5′ exon segments, have a percent sequence identity of about 70%, 75%, 80%, 85%, 90%, 91%, 92%, 93%, 94%, 95%, 96%, 97%, 98%, 99% or more to a native Group I intron-adjacent exon sequence or Group II intron-adjacent exon sequence.

In some embodiments, the monotron element is less than 500 nucleotides in length.

In some embodiments, the monotron element is capable of inducing circularization when it interacts with the terminal element. In some embodiments, a portion of the monotron element is excised post-circularization. In some embodiments, the monotron element is fully excised post-circularization. In some embodiments, a portion of the monotron element and a portion of the terminal element are retained and excised post-circularization.

In some embodiments, the precursor RNA polynucleotide comprises at least one affinity tag or affinity sequence. Affinity sequences are described in further detail herein. In some embodiments, the affinity tag comprises a polyA sequence or is a polyA affinity tag. In some embodiments the terminal element comprises an affinity tag. In some embodiments, the terminal element comprises a 5′ affinity tag or a 3′ affinity tag. In some embodiments, the monotron element comprises an affinity tag. In some embodiments, the monotron element comprises a 3′ affinity tag or a 5′ affinity tag.

In some embodiments, the precursor RNA polynucleotide comprises an internal and/or external spacer. Spacers of the present disclosure are described in further detail herein. In some embodiments, the precursor RNA polynucleotide comprises an internal spacer sequence positioned between the terminal element and the intervening region. In some embodiments, the precursor RNA polynucleotide comprises an internal spacer sequence positioned between the intervening region and the monotron element. In some embodiments, the precursor RNA polynucleotide comprises an external spacer. In some embodiments, the external spacer is positioned adjacent to the terminal element. In some embodiments, the external spacer is positioned adjacent to the monotron element. In some embodiments, the precursor RNA polynucleotide comprises internal spacers and/or external spacers. The internal spacers and external spacers can each comprise an unstructured, structured or randomly generated polynucleotide sequence. In some embodiments, the internal spacers and external spacers are at least 5 nucleotides in length and can be about 5-60 nucleotides in length. In some embodiments, the internal and external spacers are 5-60 nucleotides in length, inclusive.

In some embodiments, the precursor RNA polynucleotide comprises one or more duplexes. Duplexes of the present disclosure are described in further detail herein. In some embodiments, the precursor RNA polynucleotide comprises a 5′ internal duplex sequence and a 3′ internal duplex sequence. In embodiments where the terminal element is upstream of the monotron element, the 5′ internal duplex sequence is positioned between the terminal element and the intervening region, and the 3′ internal duplex sequence is positioned between the intervening region and the monotron element. In embodiments where the monotron element is upstream of the terminal element, the 5′ internal duplex sequence is positioned between monotron and the intervening region, and the 3′ internal duplex sequence is positioned between the intervening region and the terminal element. In some embodiments, the 5′ internal duplex sequence and 3′ internal duplex sequence are at least 80% complementary. In some embodiments, a duplex is 3-100 nucleotides in length. In some embodiments, a duplex is 5-20 nucleotides in length, inclusive. In some embodiments, the 5′ and 3′ internal duplex sequences are capable of forming, and are predicted to form, a contiguous duplex. In some embodiments, the continuous duplex has a length of no longer than about 35 nucleotides. In some embodiments, the 5′ internal duplex sequence and/or 3′ internal duplex sequence each have a GC content of at least 10%.

In some embodiments, the precursor RNA polynucleotide comprises at least one affinity tag and at least one external spacer. In some embodiments, the precursor RNA polynucleotide comprises at least one internal duplex and at least one internal spacer, for example a 5′ affinity tag and 5′ internal spacer and/or a 3′ affinity tag and 3′ internal spacer. In embodiments where the polynucleotide comprises a 5′ affinity tag, the 5′ affinity tag is positioned adjacent to the 5′ external spacer, and in certain embodiments is positioned 5′ to the 5′ external spacer. In some embodiments where the polynucleotide comprises a 3′ affinity tag, the 3′ affinity tag is positioned adjacent to the 3′ external spacer, and in certain embodiments is positioned 3′ to the 3′ external spacer.

In some embodiments, the precursor RNA polynucleotide comprises at least one duplex and at least one internal spacer. In some embodiments, the precursor RNA polynucleotide comprises at least one internal duplex and at least one internal spacer, for example a 5′ internal duplex and a 5′ internal spacer and/or a 3′ internal duplex and a 3′ internal spacer. In some embodiments where the polynucleotide comprises a 5′ internal duplex, the 5′ internal duplex is positioned adjacent to the 5′ internal spacer, and in certain embodiments is positioned 5′ to the 5′ internal spacer. In some embodiments where the polynucleotide comprises a 3′ internal duplex, the 3′ internal duplex is positioned adjacent to the 3′ internal spacer, and in certain embodiments, the 3′ internal duplex is positioned 3′ to the 3′ internal spacer.

In some embodiments, the precursor polynucleotide comprises a 3′ and/or 5′ exon segment. In some embodiments, at least a portion of the 3′ and/or 5′ exon segment is codon optimized.

In some embodiments, the precursor RNA polynucleotide described above further comprises a leading untranslated sequence and/or a lagging untranslated sequence. For example, the precursor RNA polynucleotide can comprise a 5′ external spacer that is positioned between a leading untranslated sequence and the terminal element if the terminal element is upstream of the monotron element; or between a leading untranslated sequence and the monotron element if the monotron element is upstream of the terminal element. In some embodiments, the precursor RNA polynucleotide comprises a 3′ external spacer that is positioned between the monotron element and a lagging untranslated sequence if the terminal element is upstream of the monotron element; or between the terminal element and a lagging untranslated sequence if the monotron element is upstream of the terminal element.

As described in detail elsewhere herein, the intervening region of the precursor RNA polynucleotide can comprise sequences directed to, for example, an aptamer, an expression sequence encoding a CD19 binding molecule, a stop codon or stop cassette, an intervening region comprising an untranslated region, a noncoding element. In some embodiments, the intervening region comprises an untranslated region, which can comprise one or more non-coding element, including but not limited to, a natural 5′ Untranslated Region (UTR), a natural 3′ Untranslated Region (UTR), a synthetic spacer sequence, an aptamer, TIE, a viral or eukaryotic IRES, or sequences selected from, e.g., lncRNA, miRNA, or a miRNA sponge.

In some embodiments, the polynucleotide comprises at least one mutation of a native Group I intron-adjacent exon sequence or Group II intron-adjacent exon sequence. The mutation can be, for example, at least one mutation of a native Group I or native Group II intron-adjacent exon sequence. For example, the mutation can be one substitution, at least one deletion, and/or at least one insertion of a native Group I or Group II intron-adjacent exon sequence. In some embodiments, at least one of the exon segments is less than 15 nucleotides in length. In some embodiments, the 3′ exon segment and/or 5′ exon segment comprises a Group I exon segment or a Group II exon segment. In some embodiments, the at least one exon segment is less than 15 nucleotides in length. In some embodiments, the at least one exon segment has a percent sequence identity of about 70%, 75%, 80%, 85%, 90%, 91%, 92%, 93%, 94%, 95%, 96%, 97%, 98%, 99% or more to a native Group I intron-adjacent exon sequence or Group II intron-adjacent exon sequence. In some embodiments, the at least one segment is selected from a 3′ exon segment, 5′ exon segment, or a 3′ and 5′ permuted exon segment. In some embodiments, the exon sequence or fragment is in the terminal element.

Also provided herein are polynucleotides encoding the precursor RNA polynucleotides described above that comprise a monotron and terminal element. Polynucleotides, for example, DNA templates comprising sequences encoding the precursor RNAs described above, and their uses in related methods are described elsewhere herein. Polynucleotides of the present disclosure can comprise, for example, an expression vector, DNA plasmid, a cosmid, a PCR product, dbDNA close-ended DNA (ceDNA), and a viral polynucleotide. In some embodiments, the polynucleotides can comprise a promoter segment, for example a T7 promoter, SP6 promoter or a fragment thereof.

Also provided herein are circular RNA polynucleotides produced by the precursor RNAs described above that comprise a monotron and terminal element. Circular RNAs are described in detail elsewhere herein. In some embodiments, a circular RNA polynucleotide comprises: at least a portion of a terminal element, an intervening region comprising an expression sequence encoding a CD19 binding molecule, and at least a portion of a monotron element. In some embodiments, a circular RNA comprises: (a) at least a portion of a terminal element, (b) a 3′ exon segment comprising a 3′ nucleotide of a 3′ splice site dinucleotide, (c) an intervening region comprising an expression sequence encoding a CD19 binding molecule, (d) a 5′ exon segment comprising a 5′ nucleotide of a 5′ splice site dinucleotide, and (e) at least a portion of a monotron element. In some embodiments, (d) comprises the first nucleotide of a 5′ Group I or Group II splice site dinucleotide and a natural exon sequence and (b) comprises the second nucleotide of a 3′ Group I or Group II exon splice site dinucleotide and a natural exon sequence. In some embodiments, the 5′ and/or 3′ splice site dinucleotides are distinct from the natural splice site dinucleotide(s) associated with a natural Group I or Group II intron sequence. In some embodiments, the circular RNA polynucleotides comprise additional elements, including but not limited to, a 5′ internal duplex and/or 3′ internal duplex; a 5′ internal spacer and/or 3′ internal spacer. In some embodiments, the circular RNA polynucleotide is from about 50 nucleotides to about 15 kilobases in length.

Related cells comprising the precursor RNA polynucleotides, delivery or transfer vehicles, and pharmaceutical compositions thereof are described elsewhere herein in further detail. Related methods of producing circularized RNA and related methods of treating a subject in need thereof are also provided herein.

Also provided herein are methods of identifying a monotron element and terminal element pair that allows production of a circular RNA that is translatable or biologically active inside a eukaryotic cell, comprising, for example: (i) inserting a mutated 5′ and 3′ Group I or Group II intron sequence derived from a database of native intronic sequence to form a monotron element into a precursor RNA polynucleotide described above; (ii) inserting a synthetic polynucleotide sequence to form a terminal element into a precursor RNA polynucleotide described above; (iii) transcribing the polynucleotide into RNA in vitro or allowing the polynucleotide to be transcribed into RNA by a cell; and (iv) determining the circularization efficiency of the RNA produced by the polynucleotide by identifying the amount of circularized RNA, the amount of excised intronic sequences, the amount of precursor RNA remaining after circularization, and combinations thereof. In some embodiments, the mutated 5′ and 3′ Group I or Group II intron sequence comprises at least one deletion, insertion or substitution of at least one nucleotide. In some embodiments, the 5′ or 3′ Group I or Group II intronic sequences, or combinations thereof are sequenced.

Also provided herein are methods for determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide described above, the modifying comprising: (i) mutating at least one nucleotide and/or altering the length of the terminal element and/or monotron element of the DNA sequence encoding the precursor RNA polynucleotide described above; (ii) altering the length of the 5′ and/or 3′ internal and/or external spacer sequence of the DNA sequence encoding precursor RNA polynucleotide described above; (iii) altering the length of the 5′ and/or 3′ internal duplex sequence of the DNA sequence encoding the precursor RNA polynucleotide described above; (iv) altering the length of the 5′ and/or 3′ exon sequence of the DNA sequence encoding the precursor RNA polynucleotide described above; (iv) or combinations thereof; and transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof. In some embodiments, the methods further comprise comparing the circularization efficiency of the polynucleotide with a polynucleotide comprising a native intronic sequence, or a parent polynucleotide.

d. Exemplary Elements

For means of example and not intended to be limiting, in some embodiments, a 5′ intron element comprises, in the following 5′ to 3′ order: a 5′ leading sequence, an optional 5′ external duplex, a 5′ affinity tag, a 5′ external spacer, and a 3′ permuted intron segment. In the same embodiments, the 5′ exon element comprises, in the following 5′ to 3′ order: a 3′ exon segment, an optional 5′ internal duplex, and a 5′ internal spacer. In the same embodiments, the 3′ exon element comprises, in the following 5′ to 3′ order: a 3′ internal spacer, an optional 3′ internal duplex, and a 5′ exon segment. In still the same embodiments, the 3′ intron element comprises, in the following 5′ to 3′ order: a 5′ permuted intron segment, a 3′ external spacer, an optional 3′ external duplex, a 3′ affinity tag, and a 3′ lagging sequence.

As another exemplary embodiment, a terminal element comprises, in the following 5′ to 3′ order: a 5′ leading sequence, a 5′ external spacer, an excised terminal segment, a retained terminal segment, an optional 5′ internal duplex, and a 5′ internal spacer. In the same embodiments, the 3′ exon element comprises, in the following 5′ to 3′ order: a 3′ internal spacer, an optional 3′ internal duplex, and a 5′ exon segment. In still the same embodiments, the 3′ intron element comprises, in the following 5′ to 3′ order: a 5′ permuted intron segment, a 3′ external spacer, an optional 3′ external duplex, and a 3′ lagging sequence.

In some embodiments, the terminal element sequence has a percent sequence identity of about 70%, 75%, 80%, 85%, 90%, 91%, 92%, 93%, 94%, 95%, 96%, 97%, 98%, 99% or more to an exon fragment of a sequence selected from Table 2 or Table 3.

For means of example and not intended to be limiting, in some embodiments, a 3′ intron element comprises in the following 5′ to 3′ order: a leading untranslated sequence, a 5′ affinity tag, an optional 5′ external duplex region, a 5′ external spacer, and a 3′ intron fragment. In the same embodiments, the 3′ exon element comprises in the following 5′ to 3′ order: a 3′ exon fragment, an optional 5′ internal duplex region, an optional 5′ internal duplex region, and a 5′ internal spacer. In the same embodiments, the 5′ exon element comprises in the following 5′ to 3′ order: a 3′ internal spacer, an optional 3′ internal duplex region, and a 5′ exon fragment. In still the same embodiments, the 3′ intron element comprises in the following 5′ to 3′ order: a 5′ intron fragment, a 3′ external spacer, an optional 3′ external duplex region, a 3′ affinity tag, and a trailing untranslated sequence. In some embodiments, the affinity tag is a polyA affinity tag.

In some embodiments, the 5′ intron element is located 5′ to the 5′ exon element. In some embodiments, the 5′ intron element is adjacent to the 5′ exon element. In some embodiments, the 3′ intron element is located 3′ to the 3′ exon element. In some embodiments, the 3′ intron element is adjacent to the 3′ exon element.

In some embodiments, the 5′ exon element comprises a 5′ internal duplex sequence located 3′ to the 3′ exon segment. In some embodiments, the 3′ exon element comprises a 3′ internal duplex sequence located 5′ to the 5′ exon segment. In some embodiments, the 5′ intron element comprises a 5′ external duplex sequence located 5′ to the 3′ permuted intron segment. In some embodiments, the 3′ intron element comprises a 3′ external duplex sequence located 3′ to the 5′ permuted intron segment. In some embodiments, the 5′ intron element is adjacent to the 5′ exon element. In some embodiments, the 3′ intron element is located 3′ to the 3′ exon element. In some embodiments, the 3′ intron element is adjacent to the 3′ exon element.

In some embodiments, the 5′ intron comprises a 5′ affinity tag, a 5′ external spacer, and the 3′ permuted intron segment. In some embodiments, the 5′ exon comprises the 3′ exon segment, a 5′ internal duplex sequence, and a 5′ internal spacer. In some embodiments, the 5′ affinity tag is adjacent to the 5′ external spacer. In some embodiments, the 5′ affinity tag is located 5′ to the 5′ external spacer. In some embodiments, the 5′ internal duplex sequence is adjacent to the 5′ internal spacer. In some embodiments, the 5′ internal duplex sequence is located 5′ to the 5′ internal spacer. In some embodiments, the 3′ exon comprises a 3′ internal spacer, 3′ internal duplex sequence, and the 5′ exon segment. In some embodiments, the 3′ intron comprises the 5′ permuted intron segment, a 3′ external spacer, and a 3′ affinity tag. In some embodiments, the 3′ affinity tag is adjacent to the 3′ external spacer. In some embodiments, the 3′ affinity tag is located 3′ to the 3′ external spacer. In some embodiments, the 3′ internal duplex sequence is adjacent to the 3′ internal spacer. In some embodiments, the 3′ internal duplex sequence is located 3′ to the 3′ internal spacer. In some embodiments, the affinity tag is a polyA affinity tag.

In some embodiments, the 5′ exon comprises a 5′ internal duplex sequence located between the 3′ exon segment and the intervening region. In some embodiments, the 3′ exon comprises a 3′ internal duplex sequence positioned between the intervening region and the 5′ exon segment. In some embodiments, the polynucleotide comprises a 5′ internal duplex sequence and a 3′ internal duplex sequence.

In some embodiments, the 3′ and 5′ permuted intron segments each independently comprise a Group I intron segment, a Group II intron segment, a synthetic intron segment, or a variant thereof. In some embodiments, the 3′ permuted intron segment comprises a 3′ Group I intron segment or a variant thereof. In some embodiments, the 5′ permuted intron segment comprises a 5′ Group I intron segment or a variant thereof. In some embodiments, the 3′ permuted intron segment comprises a 3′ Group II intron segment or a variant thereof. In some embodiments, the 5′ permuted intron segment comprises a 5′ Group II intron segment or a variant thereof.

In some embodiments, the 3′ permuted intron segment or element, 5′ permuted intron segment or element, or both the 3′ and 5′ permuted intron segments or elements are at least 100, at least 90, at least 80, at least 70, at least 60, and/or at least 50 nucleotides in length. In some embodiments, the 3′ permuted intron element, 5′ permuted intron element, or both the 3′ and 5′ permuted intron elements are at least 50 nucleotides in length. In some embodiments, the 3′ permuted intron element, 5′ permuted intron element, or both the 3′ and 5′ permuted intron elements have a percent sequence identity of about 70%, 75%, 80%, 85%, 90%, 91%, 92%, 93%, 94%, 95%, 96%, 97%, 98%, 99%, or more to a naturally occurring intron.

In some embodiments, the 3′ permuted intron element, 5′ permuted intron element, or both the 3′ and 5′ permuted intron elements comprise a native Group I intron segment or Group II intron segment sequence. In some embodiments, the 3′ permuted intron element, 5′ permuted intron element, or both the 3′ and 5′ permuted intron elements comprise one or more nucleotide substitutions of a native Group I intron segment or Group II intron segment sequence. In some embodiments, the 3′ permuted intron element, 5′ permuted intron element, or both the 3′ and 5′ permuted intron elements comprise one or more nucleotide insertions of a native Group I intron segment or Group II intron segment sequence. In some embodiments, the 3′ permuted intron element, 5′ permuted intron element, or both the 3′ and 5′ permuted intron elements comprise one or more nucleotide deletions of a native Group I intron segment or Group II intron segment sequence. In some embodiments, the 3′ permuted intron element, 5′ permuted intron element, or both the 3′ and 5′ permuted intron elements comprise a nucleotide substitution of one or both the dinucleotide of a native Group I or Group II intron splice site dinucleotide. In some embodiments, the 3′ Group I or Group II intron segment or the 5′ Group I or Group II intron segment comprises one, two, three, four, five, six, seven, eight, nine, ten, or more mutations of a native Group I intron or Group II intron sequence. In some embodiments, the mutations are selected from insertion, deletion, mutation, addition, and subtraction. In some embodiments, the mutations are deletions of two or more nucleotides of the 3′ Group I or Group II intron segment or the 5′ Group I or Group II intron segment, or combinations thereof. In some embodiments, the mutations are two or more deletions of the 5′ Group I intron segment at the 3′ end or two or more deletions of the 3′ Group I intron segment at the 5′ end.

In some embodiments, the native Group I intron segment or Group II intron segment sequences are selected from a sequence in Table 2 or Table 3, below. In some embodiments, the 3′ and/or 5′ permuted intron element comprise a polynucleotide sequence having a percent sequence identity of about 70%, 75%, 80%, 85%, 90%, 91%, 92%, 93%, 94%, 95%, 96%, 97%, 98%, 99%, or more to a naturally occurring intron selected from a sequence set forth in Table 2 or Table 3, or a fragment or segment thereof, or PCT/US2024/027627.

In some embodiments, the 3′ permuted intron segment comprises a 3′ Group I or Group II intron segment derived from a gene selected from a genus and/or species selected from column 2 of Table 2 or Table 3; and/or the 5′ permuted intron segment comprises a 5′ Group I or Group II intron segment derived from a gene selected from a genus and/or species selected from column 2 of Table 2 or Table 3.

In some embodiments, the 3′ Group I or Group II intron segment or the 5′ Group I or Group II intron segment are derived from a gene selected from a species selected from: Cyanobacterium Anabaena sp., T4 phage, Hypocrea pallida, Bulbithecium hyalosporum, Myoarachis inversa, Geosmithia argillacea, Coxiella burnetii, Agrobacterium tumefaciens, Azoarcus, Nostoc, Cordyceps capitata, Prochlorothrix hollandica, Tilletiopsis orzyzicola, Tetrahymena thermophila, and Staphylococcus phage Twort.

TABLE 2
Group I introns (flanked by 15 nt exons)

| | SEQ ID | |
|---|---|---|
| Species | NO: | Sequence (15 nt exon - intron - 15 nt exon) |
| Anabaena_ | 2 | gaatggtagacgctacggacttaaataattgagccttagagaagaaattctttaagtggatgctctcaaactcagggaaacctaaatcta |
| sp | | gctatagacaaggcaatcctgagccaagccgaagtagtaattagtaagttaacaacagataacttacagctaatcggaaggtgcagag |
| | | actcgacgggagctaccctaacgtcaagacgagggtaaagagagagtccaattctcaaagccaataggcagtagcgaaagctgcgg |
| | | gagaatgaaaatccgttgaccttaaacggt |
| Hypocrea_ | 3 | gagggcaagtctggtaaacgttcgctggtgccagaaatagccagccctgtaaaatggctagtcgagtgctcgatctgtagcgggtgag |
| pallida | | caccggcgagacaacctggatcggggaagcctaagggacctgtcacagggtcctatggtgatcccgagcagagccgcaaggcctg |
| | | tgtagagcacgctaaggtgtcggtcccgtcctctgcggcggggcttaaggtacgtgccaatcccctgcgaaagctgggccctaggga |
| | | gtagcacccgctgtgcgaagctctaggggacctgtgcccgccgtggcgcaggccgtccgacagtcgtcagactgctcactataatgg |
| | | ccagcagccgcggt |
| Bulbithecium_ | 4 | gagggcaagtctggtgaactgttggtgccagagatagctgcggcccgcaacggcagctagtgggtgctcgatctgtgacgggtgag |
| hyalosporum | | cacctgcgagacaacctggatcgggggaccctaagggcgccacgtgcgcctatggaaatcccgagcagagccgcaaggcctgtgt |
| | | agagcgcgccaaggtgtcggtctgacttctgtcaggcttgaggtacgtgccgatccccagcgaaagctgggcccagtggactagcac |
| | | ccgtcgtgcgaagccgctggggagtccgtgatggactttagcgacaaatcggccagcagccgcggt |
| Mycoarachis_ | 5 | gagggcaagtctggtgaactgttggtgccagatagagccgcggccctgaacggcggctagtcgagtgcccgtcagtgacgggtgg |
| inversa | | gcaccggcgagacaacctggatcgggggaccctaagggccgagaggcctatggaaatcccgagcagagcctgtagaggcctgtg |
| | | tagagcgcgccaaggtgtcggtccgtccccgcggggacgggcttaaggtacgtgccgaccccccgcgaaagcggggcctagggg |
| | | atgagcacccgtcgtgcgacgcccctggggagcatacgggctatgtataacggccagcagccgcggt |
| Mitochondrion_ | 6 | tagttgaatttgtttaacaagttccggagaatagtaatattctcatgaaaatcgggtgaattgtcaaggagacctcctacaatggagatgac |
| Allomyces_ | | ttgcagcgaagcttatatcagtgcttaatagatataagaacgttcaacgactaatcggtgagtagcactaacaataaaccggacactagg |
| macrogynus | | tggtgagtacaaccaacaataatccacccacgagcgcccgacaaccataattatggttgatgacatagtctgaacaggcaggtgactg |
| | | tctgaagagaaatttaaacgatttctcgataacataattgttgtttgggg |
| Geosmithia_ | 7 | ggcaccacaaggcgtaaaccattgctacagccgcagcaactctgcgccgaacagcagcccgaaagggtgatgtggtgccctctaag |
| argillacea | | cggtaaatgctagtctgcccccgggcaggcgacacgctcaaattgcgggaaacccctaaagctcccagctaccaagccgccggtcg |
| | | aaagacggcggtggccaggttaattgcctcgggtacggtcacaacgctgggagatgcatgggcgatccgcagccaagcccctacgt |
| | | ccccccggggatacggggaaggttcagagactagatgggcgtgggttgaccgggaggggggcgtgtccccccccgggggctta |
| | | agatatagtccggcaccgggagagatcccggtggtcaggtccacctgtaacacgggagcctgcggctta |
| Stephanoascus_ | 8 | aggctgaaacttaaaggaattgacggaagggcaccaccaggagtaaacttttacagctagccgcagtaactctgctccaaaaagcag |
| ciferrii | | cctgaaagggttagtggtgtcttcttttaattgctagtcgatatttttgtatcggcaacacgctcaaattgcgggaaattcctaaaggctttatt |
| | | accaagctaacattgaaaggtgttggtggccgagcgaaatgccctgggtatggtaaaaacataaagcatgttacaatggatgatccgc |
| | | agccaagctcctaagttcttgaaaaaaggatatggagaaggttcagagactaaatgggtgtgggtgtttgtaacgcttaagatatagtcc |
| | | gtccatgactgaaaagtcaatggtggtaaccttaaaacgggagcctgcggcttaatttgactcaacacggggaaactcac |
| Coxiella | 9 | tggggttagccctcgggcgaagctcttgatcgaagccccggtaaacggcggccgtaactataaattaaccgttgtagttacgtaaataa |
| burnetii | | acttggctatatgctggaaactccgagtatcctccagtactcatctcaactaaaattgaggtagtaaaaatctgcgaggtgcggacaatc |
| RSA_493 | | agcaggaaagactaatctttttgattagaatcctcagagactaatacgccgagcgtacttagaaaaaatccattctaagtgtgatgataga |
| | | gtccgatctctatagcgatatagagggttttcaaaagaaacctgttaatcattatcccatgattaacataacgtcaataaacggtcctaagg |
| | | tagcgaaattccttgtcgggtaaaaaatt |
| Agrobacterium_ | 10 | gagcacaggattcctaaggtaaattgggggttgcgcccggaaacgacgcaatcgatctgctcaaagtcggggaaagcttcgctggtc |
| tumefaciens | | ttccagtatgccaatcccgagccaagctccggagaaatccggtgaaggtgtagagactggatgggcagcacctaaaggccgcaagg |
| | | tccacggtgaagggacagtccagaccacgaacgcccttatccgattgagatgggggcggcgaaagccgaagcggtatgaatcctg |
| | | gggtcgga |
| Ankistrodesmus_ | 11 | atgggtgaaagcactattgcccatcatagcagctgtccacaaagcagtctgctagtcgtcgttgcaccgctttgcaaggcgcaacgcac |
| stipitatus | | ggcgagaccgtcgaattgcggggacatccttacagctcaagctaccaacttgtcgcagaaatgctggcaagggccggggtaatgac |
| | | ccagggtatggtaaaaacgcttgagattggataatccgcagccaagcaccttacccggattcaacgtccggaaaggtgaaggttcaga |
| | | gactaggtggcggtcggtcacgagacgtggcttaagatagagtccgttcgcgctgaaaagcgttcgtcgggaagaaagccttagcaa |
| | | ggccggagccagacgaagggtagccattattggctgcctcgaggtacacgggtggtgc |
| Monacrosporium_ | 12 | tttagaggaagtaaaagtcgtaacaaggtttgaagtatgacccttctgtggtgacacttacgagaagcctttgcggccccgcaaggggt |
| leptosporum | | accggccgcgactataaatgaaaggcaagccggcattaaatcgcaagtcagctcagctggctacactttcgaattgcgggaaacccct |
| | | aaagccgacctctaccaactgttcagggaaacctggatggggcctgtgctaacatcacagggtacggtaacaatgaggaggatatat |
| | | gggcaatccgcagccaagtccctaaggctctcgagctatgggaaaggttcacagactaagtggaagtgggttggggcggttgtaatc |
| | | cgccccggcttaagatatagtcgggctgtccgcgagagcagacaggatgtcacaaatcaaccgttccgtaggtgaacctgcggaag |
| | | gatcatta |
| Nostoc_ | 13 | agataactgagccttgaaggagaaatccctcaagtggaagctctcaaactcagggaaacctaaatctggtgacagacatggcaatcct |
| sp._F. | | gagccaagcccaaaaattttagatttgcgattagtcttaaattcaaaatctaaaatccaaaattacgggaaggtgcagagacccgacgg |
| | | gagctaccctaacgtgaagtcgagggtaaagggagagtccaattctcaaaacctgatttggcgattgccagcaagtagcagtgaaaac |
| | | tgcgggagaatg |
| Nostoc_ | 14 | agataactgagccttgaaggagaaatccctcaagtggaagctctcaaactcagggaaacctaaatctggtgacagacatggcaatcct |
| sp._C. | | gagccaagccgaaaaaagtcgtgagtgcagagtaaattaaaactcttaactcctaactattcggaaggtgcagagacccgacgggag |
| | | ctaccctaacgttaagtcgagggtaaagggagagtccaattctcaaaacctgatttggctattgctatcaggtagcagtgaaaactgcgg |
| | | gaggatg |
| Monoraphidium_ | 15 | ggagaagtcgtaacaaggtttccgttgaattcactagcggataacagctgccctgaaaggggggctagtcattgatagctcatgatgg |
| terrestre | | tgagggctttcaattggcaacactgtcgaattgcgggaaattccttacagttctcactaccaagccttcgtggaaacacagtcggtggcc |
| | | tagggtaatcacctagggtatggtcaaaacgtgagggattggaaaatccgcagccaagctcctaaaggcgacatgacctatggagaa |
| | | ggttcagagactaggtggcagtgggctagttgctgcgtgcaattagtttaagatagagtccggccctaccgtgaggtagcctcaagac |
| | | gaaaggactaaccagcccagagcttgaggagctgatgtgttacgtatcagtgggatcaaccgaggtgaacctgcggaaggatcat |
| Arxula_ | 16 | cagaaaagttaccacagggatgtgctaagtctattaagacaaatttacaacataacgtcccaggcatcgactcccattgatgcctagtcc |
| adeninivorans | | ggctatagcttacttgtgcttgctatagttagctctctagaagagtgtaagggcaacacgtctggatgcggggaactctcgctaggtcttt |
| | | ggtaccaaacattggcggtaacaccccaatgtagagtctgttggggcagacgggtaaaaatccaaagaatagagacaatccgcagct |
| | | gacctggccaaaagtatatttacaaagtagctaggcagttcaacgctcgctaagatgtgggttgaccattaatggtcggcttaaggtacg |
| | | ggctacgccacccgagagggtgcaactacctaccgggcgcacaacctgagaagtagttggtcatggtagtacggaactggcttgtgg |
| | | cagtc |
| Penicillium_ | 17 | gaaagttaggggatcgaagacgatttaaggaagcctcccggcctggtttccgtggaaatcaggccggagatggtatttggtcgtccaa |
| oblatum | | agtaagcctgaaaggacttgctagtctcggccgtggccgaggcgacaccgtcaaattgcggggacctcctaaagcctggactaccaa |
| | | gccgacgccgaaaggcgccggtggccggggtaatgacctagggtatggtaacaacgtccgggatgtgacaatggacaatccgcag |
| | | ccaagcgctaccgcccctccggggaccacgcgtgcagttcacagactagatgtcggtgggggatccgtctcctaagatatagtcgag |
| | | gcccagcgcgaaagcctgggagtatccgcagataccgtcgtagtcttaaccataaactatgccg |
| Staphylococcus | 18 | aactactgaaagcatAAATAATTGTGCCTTTATACAGTAATGTATATCGAAAAATCCTCTA |
| phage | | ATTCAGGGAACACCTAAACAAACTAAGATGTAGGCAATCCTGAGCTAAGCTCTT |
| Twort | | AGTAATAAGAGAAAGTGCAACGACTATTCCGATAGGAAGTAGGGTCAAGTGACT |
| | | CGAAATGGGGATTACCCTTCTAGGGTAGTGATATAGTCTGAACATATATGGAAAC |
| | | ATATAGAAGGATAGGAGTAACGAACCTATTCGTAACATAATTGaacttttagttattt |
| Cryptodiaporthe_ | 19 | gagcatgggggcgaaagactaatcgaactgttgacgcacgagagtgtgggcacaccgtatgaaaattcgttttcgcgggctgctagg |
| corni | | cagttaagagtccggccgtaaggcagggctagtaggcttcagagaggcctgcgacaccgtcaaattgcggggacaccccattagaa |
| | | agctgcgctaccgcctacggctcgaaagagctgtcaggcaccaggtgtaacggcctgggtatggtaataacgcgtgtacgcaaggg |
| | | gcaatccgcagccaatccctaacccacctctataggtggtatggaggcagttcacagactagacggcggtgggcaccgttacagaag |
| | | gtgcttaagatatagtcgagtcccgcctagcaataggtgggtagtttaacgctagtagctggttaccgccgaagtttccctcagg |
| Pleurocapsa | 20 | acggacttaaaaaaattgagccttggcagagaaatctgtcatgcgaacgctctcaaattcagggaaacctaagtctggcaacagatatg |
| | | gcaatcctgagccaagccttaatcaaggaaaaaaacatttttaccttttaccttgaaaggaaggtgcagagactcaacgggagctaccc |
| | | taacaggtcaagctgagggtaaagagagagtccaattctcaaagccagcagatggcagtagcgaaagctgcgggagaatgaaaatc |
| | | cgt |
| Spizellomyces_ | 21 | cgttgattacgtccctgcccttttccttgatggtacaaggggctaaatgtgctcccgaaaggaatgcgcaagtcttcagtggcaccgaag |
| punctatus | | gcaacactgtcaaattgcggggaagccctaaagctttgtctaccaagcagggatggaaacatttctgtggccagggtaatgacctcgg |
| | | gtatggtaaaaacggcaaagatagaacaatgggtaatccgcagccaagtcctaaacttgcacccagctggggcaagcatggatgca |
| | | gttcacagactaaatggcagtgggctctgcgatagagattattgcggggcttaagatatagtcggacccaccgcgagagcggttctcta |
| | | gatgaagctcagtgatttccaggtgcctgaagaactgatcctgagcccgagctagagtgtaaagggagcatcgttgtacacaccgccc |
| | | gtcgct |
| Chlamydomonas | 22 | caggcttagttgtaggcctcctattaggcaactaatgggctaacattgggtgaattgcatgaaagctaaggcgtaacgctatgctaacct |
| moewusii | | gcagcgaattacatcgactgatattaatataaaaatagatgtacacgttcaacgactagcgagtgatgtaaatcagtcaaataatctcgcc |
| Z17234 | | acgagcgcccgacatcttgatatttacttagacccccgcttaagtaaatatggccaagatgatgacatagtctgagcaacgtggaaaca |
| | | cgttggagccaaagataaaaaactttggcacatttctgaaacatgaaatgttcacgttaaaaaaaataaaaacgtgaaaaacataactg |
| Kirchneriella_ | 23 | ggaattgacggaagggcaccaccaggcgttaaaatagctctagcgccttagagtctcctcgagaggggaggctagtctgtagtggct |
| aperta | | cacatcggtgagagcttctatcaggctacactgtcgaattgcggggaaatccttatagctcaagctaccaagccaggatggaaacattc |
| | | tggtggcctggttaatgacccagggtatggtaacaacgcttgagattggacaatccgcagccaagcttcccaccaggtaatgctgagg |
| | | aagacggttcagagactaagtggcagtggggggtgcaaacctgcttaagatagagtccgtccctaccgaaaggtagctctgagagg |
| | | aaaggcccaaccagcctaaagctcagagagcttgcttgagcaagtgggagtaaacgggagcctgcggcttaatttgactcaacacgg |
| | | g |
| Pneumocystis | 24 | taaacggcgggagtaactatgactcaccttctgagggtcatgaaagcggcgtgaaaacgttagctagtgatctggaataaattcagatt |
| carinii | | gcgacactgtcaaattgcggggaagccctaaagattcaactactaagcagtttgtggaaacacagctgtggccgagttaatagccctg |
| | | ggtatagtaacaatgttgaatatgaatcttttgcgagatgaaatgggtgatccgcagccaagtcctaagggcatttttgtctatggatgcag |
| | | ttcaacgactagatggcagtgggtattgtaaggaattgcagttttcttgcagtgcttaaggtatagtctatcctctttcgaaagaaagagtat |
| | | attgtgctcttaaggtagccaa |
| Panellus_ | 25 | ccaccaggtgtaaacttgttagccacagttaactctgcactaaaaagcagccctaacgggtgaggtggttcagccctagagtgcgcaat |
| stipticus | | gactcttaaatctaaatgctagtctgtgcatccttgtgttgcacgggcgagaccttcaaactgcgggaaactccttagagctttcactacc |
| | | gctgctatcactgaaaagtgtagtctgcaccagggtaatgacctcgggtatggtaataacgtgaaagattggacaatccgcagccaagt |
| | | ccctaaaggtagttattgacctacgggaaaggttcagagactagatggaggttggtcaactaagttggcttaaggtatagtccgtctctg |
| | | catgaaagtgcaagaggtttgaaatgggagcctgcggctta |
| Dunaliella_ | 26 | accaggcgttaaaaagcagactcagcgcctaagagtcagtgggaaaccattggctagtgcttgggtttcattactcaagtgcaacactg |
| parva | | atcaaattgcgggaaagccctaaagctttgctaaccaagctaagtgtgaaagcactcagtggccgggttaaagacctcgggtatggta |
| | | aaatcagcaaagatgcaacaatgggcaatccgcagccaagctcctgaagccttataggcaatggagaaggttcagagactaaatggc |
| | | agtgggccaacttgttggcttaagatatagtccgtcccagctgaaaggctgtctgctagaggaagacctttctaggtctgagagctagta |
| | | gagggtaggagaagtctctcctacctggaggaaacgggagcc |
| Azoarcus_ | 27 | ctcatatttcgatgtgccttgcgccgggaaaccacgcaagggatggtgtcaaattcggcgaaacctaagcgcccgcccgggcgtatg |
| sp | | gcaacgccgagccaagcttcggcgcctgcgccgatgaaggtgtagagactagacggcacccacctaaggcaaacgctatggtgaa |
| | | ggcatagtccagggagtggcgaaagtcacacaaaccggaatccgt |
| Ditopella_ | 28 | gagcatgggggcgaaagactaatcgaactgttagctactaggcagttaagagtctcccagtaatgggaggctagtaggctctttttcag |
| ditopa | | agtttgcgacaccatcaaattgcggggacaccctgttataacgctatgataccgcgcctcgtggaaacacagagagtagcactaggtg |
| | | taacggcctaggtatggtaaaaagtcattagctaggggcaatccgcagccaagccctacgtcttcggatatgggtgcagttcacagact |
| | | aaacgttggtgggcttagaatctgtattctaggcttaagatatagtcgaatccgcctagtaataggtgggtatcaaatgaataggtgcaaa |
| | | aacttgtttatttgactacagcagaaatgctagtagctggttaccgccgaagtttccctcaggatag |
| Anabaena_ | 29 | cggacttaaataattgagccttagagaagaaattctttaagtggatgctctcaaactcagggaaacctaaatctagctatagacaaggca |
| sp. | | atcctgagccaagccgaagtagtaattagtaagttaacaacagataacttacagctagtcggaaggtgcagagactcgacgggagcta |
| | | ccctaacgtcaagacgagggtaaagagagagtccaattctcaaagccaataggcagtagcgaaagctgcgggagaatgaaaatccg |
| chlamydomonad_ | 30 | gttatagtttatttgatggtactcttactcggataaccaagcaaaaggttagtccgtgtcagtttaaacgactggcaagtcgattctattggat |
| sp._ | | cggcaacaccatcgaattgcgggaaagtccttagagccagtgctaccaagctggagtggaaacacaccggtggccagggtaatgac |
| NDem224P_ | | ctcgggtatggtaaaaacgcactggattggataatccgcagccaagctcctaagggtagcattgcctatctatggagaaggtccaacg |
| 3d | | actaagtgttggtgggctggtagcaaaccggcttaagatatagtctggccctgctgagaggcagccttgggattaaaggcttaaacagc |
| | | ccggagcccaaggggttggagcacatgctctgaccggaacaaccgaaccgtagtaattctagagctaatacgtgcgtaaatcccg |
| Penicillium_ | 31 | gaaagttaggggatcgaagacgatttagggccctcccggcgtccgccgacgccgggggatggtatttggtcgtcaaaagtaagctga |
| sabulosum | | aaaggtcttgctagtcccggccgcggccggggcgacaccgtcaaattgcggggactccctaaagcctgaactaccaagccgacgcc |
| | | gaaaggcgccggtggccggggtaatgacctagggtacggtaacaacgttcaggatgtgccaatgggtaatccgcagccaagcgcta |
| | | ccgcccgtcccgggccacgcgtgcagttcacagactagatgtcggtgggggatccgtcccctaagatatagtcgagccccggagcg |
| | | aaagcccggggaggatccgcagataccgtcgtagtcttaaccataaactatgccg |
| Ustilago_ | 32 | accaggagtaaactttcacagctagccgcagtgtctctgctccaaaaagccgcctgaaagggtcggtgcgtgttcctatttgaaataatg |
| maydis | | gctagtctgatgcgaatcaggcgacacatccaaattgcgggaaactccttagagctctagctaccgcggccccatcgtgagattaggg |
| | | tgcagcaccaggttaatggcctcgggtatggtaaaaacgctagagattggacaatccgcagccaagcctcttcgcaagaagtggaag |
| | | gttcagagactaaatggttgtgggtggcagacggtttcgcgaatgtatgaatcaatcataaacacgtgaaggaagtctgctgcttaagat |
| | | atagtccgagccacagggaaaccttgggaattttggcaacaaaccgaaacagataaataacatcgggagcctgc |
| Pleurastrum_ | 33 | ccaggcgtaaaaccaagccgtttggctctgcgccagtcgggtgtgaggagggcctcaaacaagctcctcacacgagaaacccggct |
| paucicelulare | | agtcctggtgcggcttgcatcgcgccgccgcacctgggcaacatcatcgaattgctggaaacccctaaagctcttggtaccaacctgc |
| | | atgggaaaccgtgcgggggcccactccggggaggttataatccaagagatgcaacaatgggcaatcagcagcgaagaccctgtcg |
| | | gttgggacttgcgtcccggcccatgggtaacgttcagagactaagtggtgatgggccccgatgccacccggcctcggggcttaagat |
| | | atagtccggccccggcgaaagccgatgtcgagatgagggggtgcgaccccagagctcggcagtagggatcagagttcctgctctaa |
| | | tgtnggagcg |
| Macropharyngomonas_ | 34 | agtacaaggaagcggaaggctataacaggtttaaaggcttgagtggcgcgaaagcgaccgctttgtggctacaaagtcgcgacacc |
| halophila | | gtcaaattgcgggaacaccctaaagcttcaactaccaagcgtatgtggaaacacttgcgtggccagggtaatgacctcgggtatggta |
| | | aaaacgttgaagatgaaacaatgggcaatccgcagccaagccctaaagggatttttcctaagggtgcagttcacagactagatgtcgg |
| | | tgggtagagtcgtactctgcttaagatatagtcgaggccaccgtgaaaacggtgcacagtggaggaaattcggggtaactcctggatg |
| | | gagagccgctgtgggtagacaaaagtctactggtaacctgctgtaatgcccttagatgttctgggctgca |
| Nostoc_ | 35 | agataactgagccttggaggagaaatccctcaagtggaagctctcaaactcagggaaacctaaatctgacaacagacatggcaatcct |
| sp._A. | | gagccaagcccaaaaattttagatttgtgattttagatttacgattagtcttcaatccaaaattcaaaatctaaaatccaaaattgcgggaag |
| | | gtgcagagacccgacgggagctaccctaacgttaagtcgagggtaaagggagagtccaattcttaaaacctgagctggctattgccat |
| | | taggtagcagtgaaaactgcggaagaatg |
| Nostoc_ | 36 | agataactgagccttgaaggagaaatccctcaagtggaagctctcaaactcagggaaacctaaatctggttacagacatggcaatcct |
| sp._Nephroma_ | | gagccaagcccaaaagttttagatttgcgattttagatttgcgattaatcttcaatccaaaattcaaaatcgttcgactgagcgaagccgaa |
| resupinatum | | gtctaaaatccaaaattgagggaaggtgcagagacccgacgggagctaccctaacgttaagtcgagggtaaagggagagtccaattc |
| | | tcaaaatctgatctggctattgtcatcaggtagcagtgaaaactgcgggagaatg |
| Nostoc_ | 37 | agacgctncggacttaaataattgagccttgaaggagaaatccctcaagtggaagctctcaaactcagggaaacctaaatctggtgac |
| sp._J | | agacatggcaatcctgagccaagccgaagaaagtcctgagtcatgagtgctgagtgctgagtaaatttaaaactcttaactcctaactcc |
| | | taactcataactgttcggaaggtgcagagacccgacgggagctaccctaacgttaagtcgagggtaaagggagagtccaattctcaaa |
| | | atctgatctggctattgtcatcaggtagcaatgaaaattgcaggagaatgaaaatccgttgaccg |
| Cordyceps_ | 38 | aattggagggcaagtctggtgaacgtcgctggcgccagagatagccgccctcggtaaacaaccggctagtcgagcgctcgttgcgt |
| capitata | | gacgggtgagcgccggcgagacaacctggatcggggaaggctaagggccccgcgggcctacgctgatcccgagcagagccacg |
| | | cccggtggcctgtgtagagcgcgccaaggtgtcggtcccgaggtctcctcggggcttaaggtacgtgccaatcccctgcgaaagca |
| | | gggccctgcggaacagcacccgtcgtgcgaagccgcagggagcccccctccccgggggggcgtgtgctacaggccagcagccg |
| | | cggtaattccag |
| Nodularia | 39 | acggacttagaaaactgagccttgatcgagaaatctttcaagtggaagctctcaaattcagggaaacctaaatctgtttacagatatggc |
| | | aatcctgagccaagccgaaacaagtcctgagtgttaaagctcataactcatcggaaggtgcagagactcgacgggagctaccctaac |
| | | gttaagccgagggtaaagggagagtccaattctcaaagccgaaggttattaaaacctggcagcagtgaaagctgcgggagaatgaa |
| | | aatccgt |
| Dactylella_ | 40 | gtcgtaacaaggttgaagtaagatccttctacagtgacacttacgagaagcctttgtggccccgcaaggggtacctgccgcgactataa |
| copepodii | | acaatgcaagtgggtattaaatcgcaagtcagctttagctggccacactttcgaattgcggggaaaccctaaagctcaccataccaacc |
| | | agcctgggaaatcaggttggggcccgtgctaactccacgggatatggtaataatgggatgagatgtaacaatgggcaatccgcagcc |
| | | aagtccctaaggtcttgtccagactacgggaaaggttcacagactaagtggaagtgggccggagtttgtaccctccggcttaagatata |
| | | gtcgggctgcctgtgagagcaagtgggaagtcacagttgcacttcaaaccgttccgtaggtgaacctgcggaaggatcatta |
| Prochlorothrix_ | 41 | agacgctgcggacttagataaactgggccttggtggagaaatccgcgaagtgtaagctctcaaattcagggaaacctaaggccaagg |
| hollandica | | gctagggcaatcctgagccaagctgagggatcaaccacccctaggggcgttgaccttcggaaggtgcagagactcgacgggagct |
| | | accctaacaggccgtctgccaaggcaggcgatcgctgagggtaaagggagagtccaattctcaaaacctctggcaccacgtcaggg |
| | | gcaacagtgaaagctgtgggagaatgaaaatccgttgaccg |
| Protomyces_ | 42 | ttgacggaagggcaccaccaggagtaaacgtttatgtcgcagttaactctgctccgaaaagccactcgaaagagtgtggtgtccttaga |
| pachydermus | | ggctagtcagtccgctggcaacacaatcaaattgcgggaaactccttagagctcaagctaccgctgctgcccagagatgggctgtgg |
| | | caccaggttaacgacctcgggtatggtaacaacgcctgagattggacaatccgcagccaagctccctcatgggaagaaggttcagag |
| | | actagatggttgtgggttgaacacgtttatctgaagtatttgaacggttcggcttaagatatagtccaatccaatgtggaaacgcactggtc |
| | | tgaaagatagaaaatgggagcctgcgcttaatttgac |
| Monacrosporium_ | 43 | gtcgtaacaaggtttggaatataagatccttccgtgacgcttatgagaagcctttgtggccccgcaaggggtacccgccgcgactgtaa |
| bembicodes | | aaaaggatgcaggtattaaatcgcaagtcagcctttggctggccacactttcgaattgcggggatgccctaaagccctcctctaccaac |
| | | caagctgggaaacctgcttggggcccgtgttaacggcacggggtacggtaacaacgaggttggataggtgggtaatccgcagccga |
| | | gtccctaaggcaagccgctatgggaaaggttcacagactaagtggaagtgggctggatgctccagcttaagatatagtcgggctgttg |
| | | gcgagagcgaacagatctgtcacttcatcatgattagtgcaaaaccgttccgtaggtgaacctgcggaaggatcatta |
| Tilletiopsis_ | 44 | gaggcaataacaggttaacctatcacaggcctgtaacagctgcgaggcagctagtccagcgtcgacgtctgcatttggggcagatgg |
| oryzicola | | ggtctgtttagaccttggcgcgggcgacacaacctggtacaggaaacgccccggcagcaatgccaggccgatcctgtggcgagctc |
| | | tgcgaggagccgtcgcaacgtgcggaaaggtgtgggtctttctaggaaaggcttaaggtacgtactaatccccggggaaacccggc |
| | | ctctgcgagaagagcccaaactctatgcgcagggggaggcaacggtgttgccttggtatacatgctgtgatgcccttag |
| Protomyces_ | 45 | acggaagggcaccaccaggagtaacgtttatgtcgcagttaactctgctccgaaaagccactcgaaagagtgtggtgtccttagaggc |
| inouyei | | tagtcagtacgctggcaacacaatcaaattggggaaactccttagagctcaagctaccgctgctgcccagagatgggttgtggcacc |
| | | aggttaacgacctcgggtatggtaacaacgcttgagattggacaatccgcagccaagctccctcatgggaagaaggttcagagacta |
| | | gatggttgtgggttgaacacacgtttaattgaaacagttcagcttaagatatagtccaatccagtgtggaaacgcactggtctaaaagata |
| | | gaaaatgggagcctgcggcttaatttga |
| Artomyces_ | 46 | ttgacggaagggcaccaccaggagtaaacgcaacagcttagccgcagtggctctgctccgaacagcagcccgaaagggtgaggtg |
| pyxidatus | | gtttaaaagccttgaggttcgatccgaggacctctaaagcgaaatgctagtctccctcgtggggggcgacaccttcaaattgacgggg |
| | | aactcctaaagctcagagcaccaagcttcgttccgaaagggggggtggccaggttaatagcctcgggtacggtaaaagactctcga |
| | | gatgttacaatggacgatccgcagccaaggccctacggcttcccttcgcggggtgagctacgggacaggttcagagactagatggag |
| | | gtgggtcgtcgcgcgtcgacggcttaaggtatagtccgttcgcgagcgaaagctcgatgaggtttaggtaaacccgcctttgacaaaa |
| | | cacggga |
| Nostoc_ | 47 | agataactgagccttgaaggagaaatccctcaagtggaagctctcaaactcagggaaacctaaatctggtgacagacatggcaatcct |
| sp._B | | gagccaagcccaaaaattttagatttgcgattagtcttaaatctaaaatccaaaattgcgggaaggtgcagagacccgacgggagctac |
| | | cctaacgttaagccgagggtaaagggagagtccaattctcaaaacctgagctggctattgccatcaggtagcagtgaaaactgcggg |
| | | agaatg |
| Fusarium | 48 | ggcaccaccaggggtaaactacaacggtttctcgaaagccgcagttaactctgctcctaaaagccgctctaacgagttgatggtgttcc |
| solani_f._ | | ctggctcaggcctaaactaatggctagtctcctcggaggcgacatcctcaaactgcgggaaactcctaaagctcacactaccaagcag |
| sp._phaseoli | | gcctcgaaagagagctctgtggccggggtaacgacctagggtacggtaaaaacgtgtgagatgctacagtggacgatccgcagcca |
| | | agcccctacgtgcgccagcatacggggaaggttcagagacttgacggggggggtgggctacaggcctgcctaagataaagtccg |
| | | gccccgcgtgaaagcgctgggggttacaactgcttctgcactattcgaaatactgcatcgaggccccagtaaaacgggagcctgcgg |
| | | cttaatttgact |
| Nostoc_ | 49 | agataactgagccttgaaggagaaatccctcaagtggaagctctcaaactcagggaaacctaaatctggtgacagacatggcaatcct |
| sp._I. | | gagccaagcccaaaaattttagatttgtgattttagatttatgattagtcttcaatccaaaattcaaaatctaaaatccaaaattgcgggaag |
| | | gtgcagagacccgacgggagctaccctaacgttaagtcgagggtaaagggagagtccaattctcaaaacctggccttgctaaagcca |
| | | tcaggtagcagtgaaaactgcgggagaatg |
| Pnetmocystis_ | 50 | aactatgactcaccttttgagggtcatgaaagcggcgcgaaagtgttagctagtgatccgaaaaataaattcgggttgcgacactgtcaa |
| carinii | | attgcggggagtccctaaagattcaactactaagcagcttgtggaaacacagttgtggccgagttaatagccctgggtatagtaacaat |
| | | gttgaatatgactcttaattgaggaaatgggtgatccgcagccaaatcctaaggacattttattgtctatggatgcagttcagcgactagac |
| | | ggcagtgggtattgtagagatatggggttatttatggccttatctacaatgcttaaggtatagtctaatctctttcgaaagaaagagtagtgt |
| | | gctcttaaggt |
| Trebotxia_ | 51 | aggtttccgtaggtttnantatgcggataaaagcaggcttgaaaagagtttcctgctagtacctaactttttaagtaaggtgcgacattgtc |
| gelatinosa | | aaattgcctggaaatcctgtcaagcccctggtaccgctcatcttgaggaaactctggtgtgcacctcggtgaaagtcgagggtatggta |
| | | ataatccaggggataaggacaataggcagccaactgctaaagancccgaaggccctcangcaggcagtccacagactaaatggc |
| | | aatgggtcaaggagcattaactgctcattggcttaagatatagtcggtcctcgctgaaaagcggccaatgagaggaattctctttgagaa |
| | | ggagagctcattggagtttgggtaatactctgcaatgcaaattgtatgagggcaataagcctccaaactgggtgaaacgaggtgaa |
| Bionectria_ | 52 | aattgacggaagggcaccaccaggggttaacagaccgcagtcgctctgcccctaaaagcagccgggaaactggttaaggtgggagt |
| atreofulva | | ccttggccggaaggccgaaaactgctagtcatcgaacgatggcaacaccctcaaactgacgggaacgtcctaaagctgatcaacacc |
| | | aagcaagtgtccgaaaggtgcttgtggccgagctaatagccctgggtatggtaaaagcttgatcanatgctacaatggataactcgca |
| | | gccaagcccctaagtagagcgatctatacggggaaggttcagagacttaacgggggtgggtgtggtgtanaccatgcctaagataaa |
| | | gtccgtccgcctgcgaaagcttgcgtagaaaaccgtaaaaacgggagcctgcggcttaatttgactcaacac |
| Pleurastrum_ | 53 | gattctgtgggtgaacggtgtccgtgacactactgccccagcgggtgaaagcctacgctagtcgtccttgctcgacccaaattgctata |
| paucicellulare_ | | gcaagggaacggcgagaccatcgaattgcgggaaccccctaaagcttgcaccaccaaggcatgcgggaaaccgacatgctggcc |
| SAG_4631 | | agggtaatgacctcgggtacggtaaaagcgtgcaagatgcaacaatgggcaatccgcagccaagcgccctaacaggtaatgctgag |
| | | ggtgaaggttcagagactaagtggtggtcggtgccggagcctgtgtgccccggtgcttaagatatagtccggcactacggaaacgta |
| | | gccacgagaggaaaggccagcagcgcccggagctcgtggaggctgtcggtgctccgctcaccgggantgtgactaatcgatgtga |
| | | ttttgtgggtggtgcat |
| Dunaliella_ | 54 | gacggaagggcaccaccaggcgttaacttagcagcaagctcagcgcctcaaagtcgaagggaaacctttggctagtatctgggtgta |
| salina | | gatttcacctaagtgcaacactgttcaaattgcgggaaagccctaaagctttgctaaccaagctgtcctagaaatgggatggtggccag |
| | | gtgaaagaccttgggtacggtaaaatcagcaaagatgcaacaatgggcaatccgcagccaagctcctacgggctgtcaaagcctatg |
| | | gagaaggttcagagactaaatggcagtgggcaagcatggcaatgcttgcttaagatatagtccgtcccagctgagaagctgcctatga |
| | | gaggaatgccgtaaggcaggagagctaataggaagtaagtgtctttaatcaacttacttggattccacgggagcctgcggcttaatttga |
| | | ct |
| Protomyces_ | 55 | cggaagggcaccaccaggagtaaacgtttatgtcgcagttaactctgctccgaaaagccgctcgaaagagtcatggtgtccttgaagg |
| macrosporus | | ctagtcagttcgctggcgacatttccaaattgcgggaaactccttagagctcaagctaccgcctctgtttagtgatagacagctggcacc |
| | | gggttaacgacctcgggtatggtaaaaacgtttgagattggacaatccgcagccagcttccttgagaggaagaaggttcagagactag |
| | | atggatgtgggtgaagtctttggatatgaagattttgcttaagatatagtccaatccaaagcggagacgcattggttacaacaagtagaaa |
| | | atgggagcctgcgcttaatttga |
| Planktothrix | 56 | acggacttaaagataaattgagccttgaggcgagaaatctctcaagtgtaagctgtcaaattcagggaaacctaaatctgtaaattcaga |
| | | caaggcaatcctgagccaagcctaggggtattagaaatgagggagtttccccaatctaagatcaatacctaggaaggtgcagagactc |
| | | gacgggagctaccctaacgttaagccgagggtaaagagagagtccaattctcaaagccaattggtagtagcgaaagctacgggaga |
| | | atgaaaatccgt |
| Bionectria_ | 57 | aattgacggaagggcaccaccaggggttaacagaccgcagtcgctctgcccctaaaagcagccgggaaactggttaggtgggaaa |
| ochroleuca | | gtccttggccggaaggccgaatactgctagtcatcgaacgatggcaacaccctcaaactgacgggaacgtcctaaagccgatcaaca |
| | | ccaagcaggcgtccgaaaggtgcctgtggccgagctaatagccctggagatggtaaaagcttgatcggatgctacaatggataactc |
| | | gcagccaagcccctacgtgttccgacatacggggaaggttcagagacttaacggggggggtgtggtgtagaccatgcctaagataa |
| | | agtccgtccgcctgcgaaagcttgcgaatacaaccgtaaaaacgggagcctgcggcttaatttgactcaac |
| Prochlorothrix_ | 58 | gtcaacccgtagaccatcaggggggcgtggcggaatggtagacgctgcggacttagataaactgggccttggtggagaaatccgcg |
| hollandica | | aagtgtaagctctcaaattcagggaaacctaaggccaagggctagggcaatcctgagccaagctgagggatcaaccacccctaggg |
| | | gcgttgaccttcggaaggtgcagagactcgacgggagctaccctaacaggccgtctgccaaggcaggcgatcgctgagggtaaag |
| | | ggagagtccaattctcaaaacctctggcaccacgtcaggggcaacagtgaaagctgtgggagaatgaaaatccgttgaccgtgaggt |
| | | tcgtgagggttcaagtccctccgcc |
| Dactylella_ | 59 | gtcgtaacaaggtttgaagtaagtatccttccgagggtgacgctcacgagaagcctttgtggccccgcaaggggtacccgccgcgact |
| huisuniana | | gtaaagaatgaaagcaggtattaaatcgcaagtcagctctgctggccacactttcgaattgcggggacaccctaaagcctgcctctacc |
| | | aaccgatcggggaaacctgaacggggcctgtgctaacgacacagggtacggtaacaacgaggaggatgcgaagaaatgggcgat |
| | | ccgcagccaacccctaaggcctgaaaaaggctacgggagaggttcacagactaagtggaagtgggctggaggtgttgaaaacctcc |
| | | ggcttaagatatagtcgggccctgcgcgagagcgcgggggagtcacaactggactgaaccgttccgtaggtgaacctgcggaagg |
| | | atcatta |
| Graphium_ | 60 | aaattgacggaagggcaccaccaggggttaacgtctaactgttaagccacagaagaaatctgctcctgaaagccgcccgaaagggt |
| putredinis | | gaggtggtgtccggagcgtgcgagctcgaatttaaaggctagtcctccaatgcggagggcgacatcctcaaattgcggggaactcct |
| | | aaagctttaactccaaagccggttcgcgaaagccgccggtggccaggttaacagcctcgggtactggaaaaacgtggaagatgcga |
| | | caatggacaatccgcagccaagcccctaaaggctccccgtcgagggacgcccacggggaaggttcagagacttaacggggatgg |
| | | gtattcgccgagaggcgcctgcttaagataaagtccggccctgcgcgagagcgcgggggtagcagggaactgcggcttaatttgact |
| | | caac |
| Capronia_ | 61 | ggcgaaagactaatcgaaccattaagaagccactcggtggttgagtgtaggcccgaaagggtccgcaagtgccggatcctggcgcg |
| pilosella | | acactgtcaaattgcgggaacaccctaaaactccagggaccgcctggcccccgaaagtgggccgaggcaccaggttaacgacctc |
| | | gggcacggtaatactcctggagatgtaacaatgggcaatctgcagccaagtcctacggggtcctccggggcctacggatgcagttca |
| | | cagactagatggcagtgggctcggagagcttaagatatagtcggccccctggggagacccaggcgtgtcgctaaactcgaggtgaa |
| | | ggattccgcccccggcggccacctcgagcctgaacgacgcgggcctgtgttaaacaggtcggataagcgctggtagctggttcctgc |
| | | cgaagt |
| Gaetmannomyces_ | 62 | gggagtaactatgactcaagaattggcgaacgtgagagggtcatgaaagcgggggaaaccggcgctagtgggcagccctgcctg |
| graminis_ | | cgacattgtcaaattgcgggaacctcctaaagcctcgcccaccgcggcccgccgaaaggctgggtgcagcaccaggttaacgacct |
| var._avenae | | cggggacggtaatagcggcgaggatatgtggacaacctgcagccaagtcctaagttttggggtccgacgcgatgtcaagcgccgga |
| | | cctgggccaaaggcgaccgaaatagggatgcagttcaacgactagacggcaatgggtccccggggtgacggccacggcgcttcgc |
| | | cgtcgtcccatacaaggggcttaagatatagtcttgtggtccgcttaacggcgtgtctgccacgtaaaagctcttaaggtagcc |
| Aspergillus | 63 | cgctagggatttataactgtgagtcctccaatattataaaatgttggtaatatattgggtaaatttcaaagacaacttttctccacgtcaggat |
| psudoglaucus | | atagtgtatttgaagcgaaacttattttagcagtgaaaaagcaaataaggacgttcaacgactaaaaggtgagtattgctaacaataatcc |
| | | ttttttttaatgcccaacatctttattaactagttaataaagatgatgaaatagtctgaaccattttgagaaaagtggaaataaaagaaaatctt |
| | | ttatgataacataaattgaacaggctaa |
| Arthrobotrys_ | 64 | gtcgtaacaaggtttgaagtttgttcccttccacaggtgacgctcacgagaagcctttgtgaccccgcaaggggtacccgccgcgactg |
| cladodes | | taaagaatggaatgtgggtattaaatcgcaagtcagccgccgctggcaacactttcgaattgcgggaacaccctaaagcccacctcta |
| | | ccaaccagcctgagaaatccagctggggcccgtgttaacagcacgggaaatggtcataatgaggtgggataggtcagcctttccggtt |
| | | ggcatcatggacaatccgcagccaagtccctaaggtccagctggggctacgggaaaggttcacagactaggtggaagtgggttggtt |
| | | cagcaccaacttaagatatagtcgggctctgcgcgaaagcggaggggagtcactgttccgtaggtgaacctgcggaaggatcatta |
| Geosmithia_ | 65 | gaaagttaggggatcgaagacgatttaagaagggtccgcgccccccccggtgggggccggctcggtatttggtcgttaaaagtaagc |
| viridis | | tcaaagtgtcttgctagtcgccgcgaccgcgggggcaacaccgtcaaactgcgggaacgtcctaaagcctgaactaccaagccgac |
| | | gccgaaaggcgccggtggccggggtaatgacccagggtacggtaacaacgttcaggatgcgccaatggataacctgcaaccaagc |
| | | gctaccgcctattggccatgcgtgcagttcacagactagatgtgggtggggggttgtaccccttaagatatagtcgaagcccagtgtga |
| | | aagcctgggattagttgcagataccgtcgtagtcttaaccataaactatgccg |
| Thiomargarita | 66 | aggattagatactacactaagtgtcccccagactggtgacagtctggtgtgcatccagctatatcggtgaaaccccattggggtaatacc |
| namibiensis | | gagggaagctatattatatatatattaataaatagccccgtagagactatgtaggtaaggagatagaagatgataaaatcaaaatcatcat |
| | | taaagttatagaattatcagagaatgatatagtccaagccttatggtaacatgagggcacttgaccctggtag |
| Mitochondrion_ | 67 | ctgcgttagttgaatttgtttaacaagttccggagaatagtaatattctcatgaaaatcgggtgaattgtcaaggagacctcctacaatgga |
| Allomyces | | gatgacttgcagcgaagcttatatcagtgcttaatagatataagaacgttcaacgactaatcggtgagtagcactaacaataaaccggac |
| macrogynus | | actaggtggtgagtacaaccaacaataatccacccacgagcgcccgacaaccataattatggttgatgacatagtctgaacaggcagg |
| | | tgactgtctgaagagaaatttaaacgatttctcgataacataattgttgtttggggtggat |
| Mitochondrion_ | 68 | tacctcgactacggtacttttgtgccctgctttagcagtaaccgggttaattttgagaacatctaataagacaattcaaagcctatatataact |
| Trimorphomyces_ | | gtataagttgaacgactattaataataattaactagaaaacctggctcctacttggagatgaaatagtctaaacattagtgtgaactaatga |
| papilionaceus | | aaccaataaaaggtgataatatgtattgacgatcagttaatgg |
| Genicularia_ | 69 | aagtcgtaacaaggttttgccttgagcagacgcttgcggaagccttagcagcccacaagggttcccctcgcgactatatttaaaggaag |
| spirotaenia | | gggattgaatgctagtgcctgagctgccaacttgggtgcgacatcgccaaattgcggagaactccacatttgcctcgattaccaagcgg |
| | | accgccgaaaggaccccgtggccgggctaattgctttgggtatggtcaaaacatcgaggaaaaggatgatccgcagcgaaaccctct |
| | | tgttgtacaaaagtgggtggcgttcacagactaaacggcgatgggttccagaaattcgaaacctgggcttaagatgtagtcggtttgtctt |
| | | cgaaagtggcagattgcagagtttcaccgttccgtaggtgaacc |
| Oscillatoria_ | 70 | gtagctcgtcgggctcaatacgcaagttaactaaacgcttaatagttagcattgcgatgggctgcatactaaggaaactggtatgcgata |
| williamsii | | gcctctcaaattcggggaagccggtagcgcggtaatcccgagccaagctctgaacaagagaaggtgtagagactcgatgggaggc |
| | | accctaacagttaaagctgagggtgaagggagagtccagaccacaaactggaaagataccgggcagcgaaagctgtagatggtac |
| | | gcataacccgaaggtc |
| Anabaena_ | 71 | aaataattgagccttagagaagaaattctttaagtggatgctctcaaactcagggaaacctaaatctagctatagacaaggcaatcctga |
| sp | | gccaagctgaagtagtaattagtaagttaacaacagataacttacagctaatcggaaggtgcagagactcgacgggagctaccctaac |
| | | gtcaagacgagggtaaagagagagtccaattctcaaagccaataggcagtagcgaaacgtgcggagaatg |
| Oscillatoria_ | 72 | agaaaactgagccttattggagaaatccattaagtgaccgctctcaaattcagggaaacctaactctggtaacagacaaggcaatcctg |
| sp._ | | agccaagccgaaatttcggaaggtgcagagactcgacgggagctaccctaacgtaaagccgagggtaaagggagagtccaattctc |
| | | aaaaccagaattctggcagcagcgaaagttgcgggagaatg |
| Microcystis_ | 73 | acgcaacggacttagaccaaacttgagtgctcgcagggaaacctgcgatgcagaactgctcaaattcggggaaacctcttaactggc |
| aeruginosa | | aatcccgagccaagccttggcgcgaaagtgcctcggaaggtgtagagactagacgggcagcacctaaaccccgccagcggggca |
| | | cggtgaagggatagtccagaccacaaacattcgggctaccgagtggcggtgaaaaccgtagcaggtaagaaaatccgtcgggct |
| Fischerella_ | 74 | cggtcaacggattttcattctcccgcagttttcactactgcctagtagactagctacttgtggctttgagaattggactctccctttaccctcg |
| ambigua | | acttaacgttagggtagctcccgtcgggtctctgcaccttccaaataagttgtttgttatttatcgcttgtcttttgttattcaacaattgacgaa |
| | | tgacaaatcacaaatgactaatttggcttggctcaggattgccttgtctgttgccagatttaggtttccctgaatttgagagcattcacttaaa |
| | | gagtttcttctttaaggctcaattacttaagtccgtagcgtct |
| Synechococcus_ | 75 | tcacgacctttacagtcagcggattttcattctctctgcaactttcgctgccatcaacaatcgcgatgttgagaattggactatccctttaccc |
| sp | | tcggctgaatccgttagggtagctcccgtctagtctctgcaccttctaaagctgttagttgtttagctttagcttggctcaggattgccatga |
| | | ctttaatgtttaaagttttaggtttccctgagtttgagagctgccactcgatccctttcgcgatcgaggcccagttttctaagtccgctgcgtc |
| | | taccgttccgccac |
| Synechococcus_ | 76 | cgacctttacagtcagcggattttcattctctctgcaactttcgctgccatcaacaatcgcgatgttgagaattggactatccctttaccctc |
| PCC6301 | | ggctgaatccgttagggtagctcccgtctagtctctgcaccttctaaagctgttagttgtttagctttagcttggctcaggattgccatgactt |
| | | taatgtttaaagttttaggtttccctgagtttgagagctgccactcgatccctttcgcgatcgaggcccagttttctaagtccgctgcgtctac |
| | | cgttcc |
| Chloroplast_ | 77 | agacgctacggacttaattggattgagccttggtatggaaacctactaagtgataattttcaaattcagagaaaccctggaattaataaaa |
| Gentiana_ | | agggcaatcctgagccaaatcctagttttcgaaaaagcaaaaagaaaggcttagaaagaaatgaaagaaataaaggataggtgcaga |
| brachyphylla | | gactcaacggaagctgttctaacaaatggagttgattgcgttggtaaagaaacctttctaccaaaaattccaaaaggatgaggaaaagg |
| | | ggtatatacagactgaatcaaatgattcacccacaacctgcagatcttttcacgaacggattaatcagacgagaataaagagagagtcc |
| | | cattctgcatgtcaatgccgacaacaatgaaatttatagtaagaggaaaa |
| Mycosphaerella_ | 78 | agggcaagtctggtggacgtccagtcccaatgctggcgccagaagcagtgaggccccaactcactagtcgatgcacccaccaacag |
| juvenis | | agagggcgggtgccggcaagacgacctggtacgagggaccccttaacggccgcctataggtggggccgaaggcaatctcgtggc |
| | | gacctccctcacacggaggcgtcgtaacgcgcggaaaggcgtcggtcgcgataagcggcttaaggaacgtgctagacccgaggga |
| | | aacctcgctccttctgatcagccccctctcggcgaagcagtggaggcgcccggcgaacgggtgtggacgagtcaatgaaaatcggc |
| | | cagcagccgcggta |
| Barrmaelia_ | 79 | agggcaagtctggtgaactgaactcagcttccagctggcgccagagatagtggttaaaatcactagtcgagcatcctctcccacggtg |
| oxyacanthae | | aggacgccggcaaggcaacctggattcggggaaggctaaggggcttacgggccctatgccaatcccgagcagaaattacttctgtgt |
| | | agagcgcggaaaggtgccggtcgtgagacctaccctggaacagcctaccctgtagcaagttaccctgtaacgagttaccctgtaggc |
| | | cttctcggttggggctcgcggctcgagggacgtgccaaccacctacgaaagtaggttctagtatcccgagcgtagagctaggggtatg |
| | | gagccctgtattttagggtcgtcagtcactgatagtggccagcagccgcggta |
| leotiomycete_ | 80 | ggagcctgcggcttaatttgactcaacacggggaaactcaccaggttaactacagttacgtctgggcctggaatagttatttgacaacta |
| sp. | | gtacacgcctttacttgtgtgggagaagtctcccaattttggacacaaggcctttcaggagggtttggcggtgcgacactacctggtaca |
| | | ggggacgcctcgctggagtgatccggcaggccgactctgtggcgagctcgagtaacgtcgagccgtcgcaacggcgggaaagga |
| | | gtgggttcggtggatct |
| Tilletiopsis_ | 81 | gaggcaataacaggttaacctatcacaggcctgtaacagctgcaaggcagctagtccagcgtcgacgtctgcgtttgaggcagatgg |
| flava | | ggtcttttggaccttggcgcgggcgacacaacctggtacaggaaacgccccggcagcaatgccaggccgatctgttggcgagctctg |
| | | cgaggagccgtcgcaacgtgcggaaaggtgtgggtctttctctatgaaaggcttaaggtacgtactaatccccggggaaacccgggc |
| | | ctctgcgagaagagcccaaactctatgcgcagggggaagcttgcggaatgcaaagctttggcgatgatcctttgtgacatcgataaat |
| | | gctgtgatgcccttag |
| Papulosa_ | 82 | cgatggaagttcgaggcaataacaggtcaacgcatcgcaggcctgtaatagtttaaataacgctagtccgctggatttacagcgggcg |
| amerospora. | | acactaccttgtacaggggacgcagtgctcgtcttgggagtctgccaatcctgtggcgagccctttgggccgtcgcaacgcgcgcag |
| | | ggagtgggctggggcttattggctccggcttaagatacgtgctaagctactgggtgaaaaccaggcttcgcgcttaaaaacgcggag |
| | | gatttcgtgacgaacttagaaatagactgctgtgatgcccttagatgttctgggccgcacgcgcgt |
| Chloroplast_ | 83 | cagcagccgcggtctggcgtaactaatagaagttgcaatgccgccatagcgagtgatcgttatgtaaaaaaaccctgctcataacggt |
| Chlamydomonas | | gaagccttagtaatttatctttttagggtattttacggtaataccgtgggaagtttatcaacaaaaggctgcccgcggcaaagccgcgggc |
| moewusii | | acaaaaaaagcttttttaaaacgtgttgatataaccccgtaacgacttttcttgaaagagaaagaggtgctttattagcgcctaacacgca |
| | | ggcacttacgagtcctaaatgcagcccgcggaaaccgcgggcggctatggtaataatctagcccgcggctttgccgcgggcgatgc |
| | | ctaaagtctaaagtaagttgaaggtatagtctgagcctctggaaacaggggaataatcg |
| Chlamydomonas_ | 84 | gcccattaaagtggtacgtgagctgggttcaaaacgtaaataacactgcgtgtagttgtaaaaatacaactaaaagatttggcttatatcg |
| callosa | | gcgaaaccttactatctttttaactgtacgatagatggcaatgccgagggaagttcttattctaaattaatagattaagacacctgtagaga |
| | | ctttataagtttataaaacttataagatacttcctacaaaggaaagtataatacgccaacccctactttttacctgagcttataaacaagcga |
| | | aaggctaaagcggggaggagatatagtccatgctcttatgaaaataggagggaaggtttaacaatatttgttaaactgcatgcgtgaga |
| | | cagttcggtccatatccg |
| Amoebidium_ | 85 | ggaaagtagacgcgttccggtgaagagtgttctacccttacgacaaaccgctcgaatgggccttgtgagagacaactctcacattgca |
| parasiticum | | gttggctatatgctagaaactaccgttgtattcataaatacaaaatgttatctactcttattgtatatgggagtgacaatgatggacacgagg |
| | | tacaattagcaggaaaccaaaggttacaagggtaacttagtaggatcttcagagactacacgccaaccatcttgaattagcaagatgaa |
| | | gacatagtccatagtggattagtaaagatactagtattaatattaatctactagcgtcaagctgagaaagggttagttcgatgaagacaat |
| | | cagtgttaagg |
| Chara_ | 86 | agcgaaattcctcgacaaaggggtattttctgaacagcatcaaagacaaaatctggctatatgctggaacctccgagtgacgcaagca |
| vulgaris | | aaatgcgtccggacaatcagcagggaaggaagtcatacatgtgttgatacatgcgctgcgcccctcaacgactacacgccagacatc |
| | | cctgcttgggatgatgatatagtctgaacttcgtggcgacacgaaggaggaaggcctcggttgaaatgatgttcgaagtttatttcaatta |
| | | acacaattgtgtcgcataagtagcgacctgcacgaatggtgtaacg |
| Mitochondrion_ | 87 | cgacgcgcgtgttcggcaccttgaggcgctacgcaagcgtagcgccgattcatcggggtatttttatagtgatataaaaaaaagaaaaa |
| Chlamydomonas_ | | ggctaaaagctggaaaatctataatatgataatcagctggaacaaaagtatgtaataagtaatagttattatatataaaagattcttcaacg |
| moewusii | | attaaaagcctagctcttttaataaagagtatgatgtagtctgatc |
| Pyrictlaria_ | 88 | atttagccgaggctaaaaaaatattctgatttggtctggttatatgtcgcaaattcctggaaaatctttataaggtaatcagcaggaaaactt |
| higginsii | | aattaatttagtgattaaactcttcatcgactaaacgtgacaatttaatttttttattaaataagagatagtcaaaatttcgatgtgaatcgaata |
| | | atatttattataagagcaccaggttcccagagcgctaggcgattattataatttatattagccctcccttggaatcag |
| Stigeoclonium_ | 89 | agtgaacggcggccgtaactatgacggtcaactttagggccgtctttaaagataactttttgctggaacctccataaaaagagcaatcag |
| helveticum | | caagagattatattatttgttctaataatttcaacatctgtataaatttccgtctacattaaatggcttactttacgaatgtcttcctttatgggatg |
| | | ccatgagaatacaaaaaggccaaaaaaatcaaagttttgaaactcttgaagacgcgcaaaattatgtacgagattttgttgcgaacaaat |
| | | aaaaatagtttcttcagagactactggttatccgattttaaatattttagtatttaagattgaagatatagtccgatattatatgaatgtgtaataa |
| | | ataccttgcctaaggtagcgaaattctttgtcgagtaag |
| Neochloris_ | 90 | ggcagctgtaactatgacagttctaaggttaagaaatgctagctttagtaaaatcaaactatttgctgggaacccgtaataaaggaaatca |
| aquatica | | gcaggtaactctataataatttatttatagagcacctcagagactatacgtttgacgttttgatgaaaacatcaactttaagatagagtccag |
| | | cgttttttgaaagaataacgattatctgagcgaaattcattgtcgagtaagtttcg |
| Pedinomonas_ | 91 | caggctgatcttccccaagagttcacatcgacgggaaggtttggcacctcaactggggtctatcaatgtgaattgataatgcacaagtta |
| tuberculata | | gctatatgctggaaaatccggaggatccaaaaacccaccctaattttgttaggtcggaaaagttttttggttcggacaatcagcagggaa |
| | | gaccatgtgcccctcaacgactacacgctgacctacttaaaaaaaaagtagatgatatagtctgaacttcaaggcgacttgaaggtattc |
| | | atttttctgtgaacgaactgcaactagcagcacgcgaaaaggaacacgttttcgattgcattcgaaaattaacgtctttgcgatgtcggct |
| | | catcgcaacctggggcggtagtacgtcccaagggttgggctgttc |
| Chlamydomonas_ | 92 | caggctgatcttccccaagagttcacatcgacgggaaggtttggcacctcgacatagggggtcgctttcacgcaagtgaaggctgtaa |
| starrii | | attagctgtatgctggaacttacctattaggtaaaatcagcaggaagtgaagcacacgtgtttagctcctcaacgactacatgctaacact |
| | | cttgtccttgcgtcctgtgccgcgccgcgccttcttgtccttcgccgaaggcgcggacgcgaaggcgaaggcaaggacgcgaaggg |
| | | gaaaggacgcaagagcagagtgatgatatagtctgaactacgctttaacggcatagaccaacttagttggtttctttctagataacctag |
| | | gagggataacatatttgcgatgtcggctcatcacatcctcggtctgtagtaggtccgaagggttgggctgttc |
| Chara_sp. | 93 | gatcttccccaagagttcacatcgacgggaaggtttggcacctcaaatcggggcaatagtccagtaatgggaaaaaaagtagagaat |
| CL_20021 | | ttggctatatgctgggacatccgtagtttctaatctaaaaaaataagtgacaatagcgtaggaaatgaattgaatgaattcggattatcagc |
| | | agggaagtttcttcaagatacccctcaacgactacacgccatgtatcctttgcttttcaaggataatgatagagtctgagcttttaggtaact |
| | | aaaaggccactattgagtgttagtggcgttaaaccatacatgtcgaaattccccggaaaattgataatgtttaattaacacattgcgatgtc |
| | | ggctcttcgccacctggagcggtagtacgttccaagggttgggctgttc |
| Chara_ | 94 | gatcttccccaagagttcacatcgacgggaaggtttggcacctcaaatcggggcaatcgtccagtaatgggaaaataaagtagagaat |
| corallina | | ttggctatatgctgggacatccgtagtttctaatcaaaaaaatcagtaacaatagcgtaggaaatgaattgaatgaattcggattatcagca |
| | | gggaagtttcttcaagatacccctcaacgactacacgccatgtatcctttgcttttcaaggataatgatagagtctgagcttttaggtaacta |
| | | aaaggccactattgagtgtgagtggcgttaaaccatacatgtataaattacccggactaaaaatccataaaaaaatcaaattgagaatgtt |
| | | taattaacacattgcgatgtcggctcttcgccacctggagcggtagtacgttccaagggttgggctgttc |
| Selenastrum_ | 95 | cctcttacacggataaccgtataatacatgcggaagaagtactcgcgttcctagtagccttaaaaggctgcgacattgcaattgcctgaa |
| capricornutum | | ccctgttaatctcaccagtaccgctgctgtcccgaaagggaccacgcagcaccttggcaacgcccgagggtgtggtaaaaacctgaa |
| | | gagtaggggcgatgggcagccaagtcctgttggtagccaatacctacggatgcagttcacagactaaatggtaatgggttcttgagttt |
| | | gactacacaagagcttaagatatagtcggtgggtgccaaaaggcagaccgcaggaggacaacaaccacacgttgtctgagagcctg |
| | | tggtggacagtaatagttgggtgcaaccaaccggtctgaccgtctgctcccggaacaaacgagtaattctagagct |
| Chlorella_ | 96 | tactcggataaccgtaaggattnttgcgggagggaancgaaagcctgccctagtggctcgaaaagcgagctgcgacactgtcgaatt |
| trebotxioides | | gcccggacgtcctgctacgctggagggaccgtctgggtcgggagaccggcctggacactcgtggcgaaagcctcgaggtacggta |
| | | acactcctcccgctaggggctatgggcagccaagctctaagtcgctctcgcagcgatacgagtgcagtccacagactagatggcagt |
| | | gggtatcgctcatcgaagcgatgcttaagacatagtcggtccccaccgagaggtggttcgacggaggatcgaggcgaccgcctcgt |
| | | gagagccgccgaggtcgggcgaggtctagctagacgacccgccgacggagcacacgagtaattctagagc |
| Delitschia_ | 97 | gaacgaaatttaggggatcgaaaacaattttacagttaaacgtgacagtttctggtcgtcgaaagtgcgtaagcactaatctgccccccc |
| didyma | | cggggggggcgacatcatcaaattgcggggacccctgggaaccccagctaccacacacggttgaaaggcactgtgccaacttcg |
| | | gagagagaccgaantgaagtaaaaacgctgggtcaggatctattcctacaggaaatccgcagccgacgccctaaaccccctcccgg |
| | | gggcatgggtgcacttcacagactaaattgtgatgggtarggggcaytscctcctgcttaagatttattcgggcccggtgcgaaarcgst |
| | | ngnaacaatcttaanaatycgncagataccgtcgtagtcttaaccgtaaactatg |
| Coelastrella_ | 98 | gaattgacggaagggcaccaccaggcgttaataagagctgctcagctttagcgccagtggcttggaaacaagctgctagtcacactcc |
| multistriata | | tatttgtgagtcagagacttgctatagcagtgtggcaacatcctcaaattgctgggaagccctaaagctgctacataccaaagttggttgg |
| | | aaacaactgactggccaggttaacaacctcgggtatggtgacaactgtagcagatgaagcatgctcacatgctgaagtgggttatcag |
| | | cagccaagtcctaaacgtgctcggtgtgttcaactctaccgcaaggtagcttattccaagacacaccaagcatgcatggatgcagttca |
| | | cagactaaatggggggggccctcgaagcttaagatataggagcctgcggcttaatttgactcaacacgggaaaacttaccaggtcca |
| | | g |
| Naohidea_ | 99 | gtctaggttcgggggggagtatggtcaagcgttgcggccatgaagagtgacaaggtaacttgttgctagtctatttaataggcaacacc |
| sebacea | | gtgcaaattgcggcaacacccttaagcttgttaacaccaagcacggtaccgaaaggtacgtgtggccagatgtagtgatctgggtatg |
| | | gttatagtttatcangtttgggcaagcacgcagccaagttcttactcgttagcgagagaatgcagttcacagactaaatgccggtgggta |
| | | ggaaaaagcaatttcattgcaaaagtgttcttatcaaatcctacttaagatatagtcgggccacgtcgaaagatagtggattagcctgcgc |
| | | aaggctgaaacttaaaggaattgacggaagggcaccaccag |
| Sclerotinia_ | 100 | gaaacttaaagaaattgacggaaaggcaccaccaggcgttaactgcagtaactctgcgcctaaaagcagctcgtaagagttggtggta |
| trifoliorum | | gccttcaaatatgctagttggaaatcagctataccttcaaactgcggggaactccttaaaaactcaactactaaacctcaattgaaagatt |
| | | gtggtggccagctgattctgggtagagtaataatgttgagaatttggacaatccgcatccaaccctctaaggttttaaactacgaggaag |
| | | gttcagagactaaatgtaggtaggtagcgtcgctacttaagatatagtccgcctcgagattaacgtctcaagaataacaatgggagcctg |
| | | cggcttaatttgactcaacacggggaaactcaccagg |
| Pseudallescheria_ | 101 | ggaagggcaccaccaggggttaacgtctaactgttaagccacagaagaaatctgctcctgaaagccgcccgaaagggtgaggtggt |
| boydii | | gtccggagcgtgcgagctcgaatttaaaggctagtcctccaatgcggagggcgacatcctcaaattgnggggaactcctaaagctttc |
| | | actccaaagccggttcgcgaaagccgccggtggccaggttaacagcctcgggtactggaaaaacgtggaagatgcgacaatggac |
| | | aatccgnagccaagcccctaaaggntccccgtcgagggacgcccacggggaaggttcagagacttaacggggatgggtatgcgcc |
| | | gagaggcgcctgnttaagataaagtccggccctgcncgagagcgcgggggtagcagggaacctncggcttaatttg |
| Sporobolomyces_ | 102 | acttaaaggaattgacggaagggcaccaccaggagttaacgcaagacaaagctctgctcttcaaggcgaaccagaaatggtttgtggt |
| sasicola | | gtcccttgtaacattcgccagtactctacgcgagggcgacactttcaaattgcggggaactcctgagagccctcagctaccaagtcgaa |
| | | gcgctgaaaagcctttgatggccagattaaaaatcttgggtacggtaacaacgttgagggattggatgatccgcagccgcctccccttc |
| | | aatggggcgcggttcagagactaaacggaagtgggtccactctaaggatctaagatatagtccatggcttgaggaaactcaaag |
| Nigrosabulum_ | 103 | ggctgaaacttaaagaaattgacggaagggcaccaccaggggtaaaatccgcagaagctctgcccctaagagcagccgggagacc |
| globosum | | ggtcggtgggagaaaacagcccgggcgccgcagagcgcgaaccaactgctagtcggcccccccgggggtccggcgacatcctc |
| | | aaattgcggggaatccctaaagccgcgtgctaccaagtgggccgccgaaaggccgcccgtggccgggtctaacaaccccgggtac |
| | | gttacaacgcaccggatgacaacaatgggtgacctcgcagccaagctcctacacggcttgccgcgcaggaggaaggtccagagact |
| | | tgacggggatgggtcccgccgccagccggcgtggcctaagataaagtccgtctgcgcgcgaaagcgctgcagagccctaaacgg |
| | | gacgtgcggcttaatttgactcaac |
| Beauveria | 104 | attgacggaagggcaccaccaggggtaaaccgcttcatgcagcccgcagtagctctgctcccaaaagcagcccgaaagggtcaagt |
| bassiana | | ggtgttcctgtccgcgctccggcgcggcgaaccaaagtgctagtctctccggaggcgacaccctcaaattgcgggaaacttctaaaac |
| | | cagtgtcaccaagccggcgtcgaaaaggcgtcggtggccggggcaatgacctagggtacggtaaaagcacactggatatatggac |
| | | aatccgcagccaagcctcgtcacggggaaggttcagagactaaatggggggggtagtgtgctggaggattcgtcctccgagcgctc |
| | | gctcacacactgcttaagatatagtccgcccgcgctggaaacagcgcggattggcaagctcaaataaacgggagcctgcggcttaatt |
| | | tgactc |
| Mycoarachis_ | 105 | ggctgaaacttaaagaaattgacggaagggcaccaccaggggtaaaacgccgcaagctctgctccggaaagcagccaggagactg |
| inversa | | gtcggtggtgccttcagaacacaagtgctagtcaccctccgcggtggcgacaccctcaaattgcgggaaatccctaaagccgcgtgct |
| | | accaaccggaccgccgaaaggcggccggggctgggctaactgccacaggtatggtcacaacgcaccggatgagcggccgagag |
| | | gccgcgaagtgggtgatccgcagccaagctcctacgtagaggtcccctctatacggagaaggtccagagacttgacggggggggt |
| | | gcggcagcggccgcgccgaagataaagtccgtccggcgtgcgaaagcgccccggatccctaaacgggagcctgcggcttaatttg |
| | | actcaacacggggaatctca |
| uncultured | 106 | ggaagggcaccaccaggagttcgccgaactgcgatttattagcagactctgctcctgaaagccgcccgtagaagggtcggtggtcgt |
| soil_fungus | | ccaggcttcgagccgaacaagtgctagtcacgcgagtggcaacatcgcccaactgcgggaacgtcctaaagctctacctaccaaccc |
| | | gccgtcgaaaggcgtcgggggcccggttaatgcccggggtacggtcacaacgggagagatatacaatggataatccgcaaccaag |
| | | cccctacagcaatctgccacggggaaggtccagagactaaacggcggtgggctcacggggagcttaagatatantccgtccgggg |
| | | atcagcgtcccacgga |
| Kirschsteiniothelia_ | 107 | gaaattgacggaagggcaccaccaggcgttgtggcagactctgcgccgagatgcggccccccaggcggggggtgggtggtgccg |
| aethiops | | tccttaaatgccagtctccgacgagcctcatcgtcgggggcgacatgccgaaattgcggggacatcccaaggctccgaccaccaggt |
| | | cgccccccgaaaggcgggcgacgaccgggttctccccgggtagggtgggcacggtagagctggggatcactcgcagccgagcg |
| | | ccttccgttccatcggggccccgagggcccggggggaccacggcgccgggtccagagactagacaggcatggggggccccgtt |
| | | gcggaggggcccgcttaagatatagtccacgagcatccgaggatgcctccatcagggagcctgcggcttaatttgactcaacacg |
| Protomyces_ | 108 | tatggtcgcaaggctgaaacttaaagaaattgacggaagggcaccaccaggagtaaacgtttatgtcgcagttaactctgctccgaaaa |
| lactucae- | | gccactcgaaggagtgtggtgtccttagaggctagtcagtacgctggcaacacaatcaaattgcgggaaactccttagagctcaagct |
| debilis | | accgctgctgcccagagatgggttgtggcaccaggttaacgacctcgggtatggtaacaacgcttgagattggacaatccgcagcca |
| | | agctccctcacgggaagaaggttcagagactagatggttgtgggttgaacacacgtttattgaaacatgttcggcttaagatatagtcca |
| | | atccagtgtggaaacgcactggtctaaaagatagaaaatgggagcctgcggcttaatttgactaacacggggaaactcaccaggt |
| Bensingtonia_ | 109 | accaggagttaacaaacaggctctgctccaaacagtagctcgaatagagtacggtggtcgctgtcgtttaaatactagtctgtttataaca |
| ciliata | | ggcaagatcttcaaattgcggggaagtactggggctctaatctaccaagtcattgtctgaaaaggcatgatggcctgaggtaataactc |
| | | aggatatggttacaacgattagagatgttacaatgtatgattcgcagcccaatccctcaatatgggatcaggttcagagactaaacggag |
| | | atcgatctaggcattgaactaggtttaagatatagtccattattctgcttaacagcaattgaatgtattaattgggagcctgcggcttaa |
| Pyxidiophora_ | 110 | acaccaggggtaaacttcgaactgtagccgcagtcgctctgctcctaaaagcagccgggaaaccgtcaggtggtaagctccttgccc |
| arvernensis | | aggtgggcgaagtattgctagtcgtcgtaagggcaacaccctcaaattgacgggaagtccctaaagccagctgagaccaagcgagta |
| | | tccgaaaggtgctcgtggccgagctaatagccctgggtatggtaaaacagcagctggatattacaatgggtaatccgcagccaagctc |
| | | ctaaacagatttatactgcacggagaaggttcagagacttgacggggggggtgcgtaattggtttttattgaactgcttacacgtgctta |
| | | agataaagtccgttgctgctgtaagcaggacaggtcttggtaacctttgaccattaaagggagtgcggctt |
| Geomyces_ | 111 | cttaaagaaattgacggaagggcaccaccaggagtaaacttagtttcgtcgcagcaaactctgctccagaaagcagcccgaaagggt |
| pannorum_ | | tagtggtgtcccaggcctcgggccgaattaaatgctagtctgccacggcaggcgacaccctcaaactgcggggaactcctaaagcttc |
| var._ | | taggtaccaagccggcgtgcgaaagctgccggcggccagggtaattgcctcgggtaccgccaaaaccctagaagatgctacaatgg |
| pannorum | | acaatccgcattcaagcccctaagggaccacggtcctacggggaaggttcagagacttgatggggggggtttgagaatactcaggc |
| | | ttaaggtaaagtccggccgctggagaaatccatgcggattggtaaataaaacgggagctgcggcttaatttgactcaacacgggaaac |
| | | tcaccaggtcca |
| Penicilliopsis_ | 112 | tcgcaaggctgaaacttaaagaaattgacggaagggcaccacaaggcgtaaacttttaccgctagccgcagtaactctgcgccgaac |
| clavariiformis | | agcagcccgaaagggtgatgtggtgaccctctaaacagtaaatgctagtccagcacggctgggcgacacgctcaaattgcgggaaa |
| | | ccccttagagcctctagctaccaagccgccggtcgaaagacggcggtggccaggttaattccctcgggtacggtgataacgctagag |
| | | gattgggcaatccgcagccaagcccctacgttccgcagggaatacggggaaggttcagagactaaatgggtgtgggttgggtcctcc |
| | | gggggaggacaccagcttaagatatagtccggcgccaggggaaaccatggcggttcaggtatacctgtcttaacgggagcctgcgg |
| | | cttaatttgactcaa |
| Dactylella_ | 113 | cttaaaggaattgacggaagggcaccaccagatgtaaaccgtagttaactctgcatctgaaagccgtctcgggagtagcatcctgaag |
| cylindrospora | | gttgaatctggccctgttcgcaatggctagtccaagctttttcagctgggcaacaccctcaaattgcgggaaactcctaaagctgctgaa |
| | | caccaaacctgaccggtaacggttgggtggccgccctgaaagggtgcgggtatggtaacagagtcagcagattgctcgccagcggg |
| | | ccagcaaatggacgatccgcagccaagcgaccggttcaaacccaaccggttgaaggttcagagactagatggggggggttttcaaa |
| | | aacttgaaggcttaaggtatagtccagccgttccagcgaaagcaaacgggtcaggcaatgggagcctgcggcttaatttgactcaaca |
| | | cggggaaac |
| Graphium_ | 114 | ggaagggcaccaccaggggtaaactgaagccgtatcgctctgccccgaaaagcgagcccgtaacgggttcggtggttgccggcatt |
| basitruncatum | | cgctaggtccccgtccaggcgggggcgacaccctcaacgtgcggggaaccctcgaagggcaccactcctaagtaacggtcgaaag |
| | | acgcgctatggcggcgccgtaatggccgccggtacagggaaaacgtggtgcttaggaggcgattcgcagccaagctcctaccccg |
| | | cctccggggcgcagggagaaggtccagagacttgacgggggggggtgngcgcgcagcccgcgccnnngngcctaagataaa |
| | | gtccgtgcctgcgcgagagcgcgggaatgctctacagcagagccccgaacaggggatgcggcttaatttgactcaacac |
| Graphium_ | 115 | ggaagggcaccaccaggggtaaactgcaaagccgtatcgctctgccccggaaagcgagcccgtaacgggtccagtggtcgccgg |
| penicillioides | | cattcgctaagtctgcacgcgcagcggcgacaccctcaaattgcggggaaccctcgaagggcgtcactcctaaggacggtcgaaag |
| | | gcgcgctatggngcagcgtaacggctgcnntacagggacaacgtgancctaggaggcgattcgaagccaagctcctaccngcgc |
| | | cccggcgccgtagggagaaggtccagagacttgacggggggggtaggtcctcgcgggcccgcctaagataaagtccgtgcctnc |
| | | gcgagagcgcgggaatgctccaagcagagccccggaaaggggctgcggcttaatttgactcaacacgggg |
| Scotiellopsis_ | 116 | ntcaacacgggaaaacttaccaggtccagacatagtgtaacgatagctgctcagctttagcgccagtggcttggaaacaagctgctag |
| oocystiformis | | tcacactcccatctctaggcaagacagcctgcagtatggatgtggcaacaccctcaaattgctgggaagccctaaagctgctacatacc |
| | | aaagcttgttggaaacaactagctggccaggttaactacctcgggtatggtgacaattgtcccagacatagggaggattgacaagattg |
| | | agagntntttctng |
| Bensingtonia_ | 117 | gcgttgattacgtccctgcccttttttcattttaaacgcttgatgtacaaggggttcaaagtgtcttggtaaaagatgctagtagtgaacacg |
| thailandica | | gtattactatttgtcaacttgccagaccgtcaaattgctggaaagcctaaagctttaactaccaagtcaagacagaaatgtactgatggct |
| | | gggtcacgacccagggtaaggtgaaaacgttaaagatgtttaacaatgggtaatcagcagccaagccctaaggtgtcttgtacactag |
| | | ggtgagttnacagactagatgtcggtcggcttttcgattaatcgaaaaagcttaagatatagtcggcccaagtctgaaaggactttggat |
| | | aaagtgttgtacacaccgcccgtcg |
| Gonatozygon_ | 118 | accttatcatttagaggaaggagaagtcgtaacaaggttttgcctcgagcaaacgcttgcggaagccttagcagcccgaaaaggttcc |
| aculeatum | | cctcgcgaccaaatccgaaggaaggggattgaatgctagtgcccaagttgccaacttgggcgcgacatcgccaaattgcggagaact |
| | | ccactatttcgcctcgattaccaagcggtacgccgaaaggactccgtggccgggctagttgcctcgggtaaggtcataatatcgaggat |
| | | gagggaagatccgcagccaagccctcgcgccaaacgctcgtgggtgctgttcacagactaaacggcggtgggttccggactctaga |
| | | agcctggagcctaagatatagtcgttcgcctcgaaagagcaatctgcagagattcaccgttccgtaggtgaacctgcagaaggatca |
| Mesotaenium_ | 119 | aacaaggtttggctcgatcgtggatcctttcgcggttgaaactaccgaagccttagctgcccgaaagggtgacctccgcgactcgcaa |
| caldariorum | | aacaaggagaggtctcaaaagctagtggctcctagctgctgctcgtgcagtgagtgcgacatcaccaaattgcgggaaatccctaaag |
| | | ctttgactaccaagtgcggcgccgaaaggctcgcatggccagggtaatgacctcgggtatggtaacaacgtcaaagatgcaacaatg |
| | | ggctatccgcagccaagtcctcgccgcttcgcagcggttggatgctgttcacagactagatggtggtgggttctcggatgcgtcccgg |
| | | ggcttaagatatagtcgggcctgcttggaaacagcgggaaatcgcagatgcattcaccgttccgtaggtgaacctgcagaaggatcaa |
| Statrastrum_ | 120 | aacaaggttccgtttatgatccttctcgccccgttatgggttcgagtcgcacggaagccttagcactatgtgcattgtattttaatacgctgc |
| sp._M752 | | acccgtgctagtgccaatgatgatgtcattggtgcgacgtcatcaaactgcgggaaagctcctatatggcatgggataccaagcacgg |
| | | catcacacattgtgtgtgatgagtgtggctgagttaatgcactcgggtaaggtaacaacccccatgtcaagaggaccgacctgcagcc |
| | | aagcctttggttataccaagggtgctgttcacagactaaatgatgacgggttgctaatcacaaagcgtgattgcagctcaagatatagtc |
| | | ggactcactctagagtgagacaacacgtaataccttcccgcacgtgaattcaccg |
| Cylindrocystis_ | 121 | tttagaggaaggagaagtcgtaacaaggtttcattgcgtccttctgcaagtttgctactgaagccttagctgcctgaaaaggtgaccttga |
| brebissonii | | cgacttgataaagaaacaaaggtcttaaatgctagtgttcaccctgagtggacgcgacatcaccaaattgcgggaaaaccctaaagctt |
| | | tgactaccaagtacacgtccgaaaggagtgcatggccagggtaatgacctcgggtatggtaataacgtcaaagatgaaacaatgggt |
| | | aatccgcagccaagtccttgcttgtcatggatgccgttcacagactaaatggtggtgggtttgcgaagcaagcttaagatatattcgggc |
| | | ctctcggaaaccaaaagggggcagtttcnccgttccgtaggtgaacctgcagaaggatca |
| Closterium_ | 122 | gaggaaggagaagtcgtaacaaggttgcccctgggcacgatccttccgaggaccccagacggaagcctgagctatctctgtccgcg |
| spinosporum_ | | gcgtggagagagagagagctaggtgtggctcatgccataccgacaccgtccaactgcgcgagagttccagaccctggccggtacca |
| var._ | | agcgctctcaagagcgtggccggagaagctgtcatccatcgcggctctcttcgggtatggtcataacccggcaccgggctcaaggat |
| spinosporum | | aatccgcagccaacccgtctcaggcgggaccgttcacagactgaacgacggtgggtccatgcgtgcgtgggcttaagatacagtcg |
| | | gcatctccccatttaagggagattcgagcaagcacagtcggcctgcagcgatttcaccgttccgtaggtgaacctgcggaaggatcatt |
| | | g |
| Closterium_ | 123 | gaggaaggagaagtcgtaacaaggttgcccctgggcacttccttccgacaagggacccagacggaagcctgagcaccgcccgctg |
| spinosporum_ | | gcgcacagcgggctcggtgctaggcggtgcgcgcgtgcgcgcactgtcgacaccgtccaactgcgagagagttcccgaccgcgg |
| var._ | | atggtaccaagcgcctcagagcgtggccggaggcggagcgccgctcccccctcygggtatggtcacaacccatccctggcagacg |
| malaysiense | | gatgatccgcagccaacccgtcgcgtcgtgacgggccagttcacagactgaacgacggtgggtccgggtgtcgcaagccgcgaaa |
| | | cccgggcttaagatacagtcggcggctccgggagaaccggagtcccaccccctcccctcgcagaggacttcaccgttccgtaggtg |
| | | aacctgcggaaggatcattg |
| Phialophora_ | 124 | ggtcatttagaggaagtaaaagtcgtaacaaggtttcaccacggagaccatgcatcccctgagggaggcagcgcgactgtaaataac |
| gregata_ | | gctgccgtatgcaagtcaggctaccgctctggcaacacgatcgaattgcggggacctcctaaagcttcactcactacctacccccgaa |
| f._sp._ | | aggaggcagcacagttataacagtgaagatgttacaatggacgatccgcagcggagaccctacagcgcaagccacgggtagcgttc |
| adzukicola | | acagactaagtgattgtgggtagtacggtatactacttaagatatagtcgaacccttactgaaagtgaggggtaaaggtccgtaggtgaa |
| | | cctgcgga |
| Dactylella_ | 125 | gtcgtaacaaggtttgaagtaagtttccttccataggtgacgctcacgagaagcctttgtggccccgcaaggggtacctgccacgactg |
| gampsospora | | taaagaatggatgcaggtattaaatcgcaagtcagctagcagctggcaacacttttgaattgacggggacaccctaaagcccacctcta |
| | | ccaaccagctggagaaatccggttggggcctgtgttaacagcacagggtacggtaacaacgaggtcggataagccagccctctggc |
| | | acaatgggtaatcctcagcccaagtccctaatcttttcgttcagaaaaagacatgggaaaggttcacagactaagtggaagtgggttggt |
| | | tcctgaacccaacttaagatatagtcgggctctgtgcgaaagcagagaggagtcactgttccgtaggtgaacctgcggaaggatcatta |
| anamorphic_ | 126 | gaggaagtaaaagtcgtaacaaggttttgctaagcaacagtccttctgtagcagagcttaacggagaccttgcgccaaaaggcgacta |
| fungus_ | | taaataatcggcagcaagtcagtatcactggcgacacaatcgaattgcggggacgctttaaagctcgacggtaccaaccgctggaaat |
| JCM_8069 | | cccagcggggctagcatgctagaggtcagaactcgtcgagatgtacaataagcaatccgcagcggtccccttcgaggggccgtcca |
| | | cagactaagtggttgtgggtaggacgtctgttctatctaagatatagtcgggccttgcgagagatcgcaggggtttctgcgtccgta |
| Encoelia_ | 127 | gcgtataggggcgaaagactaatcgaaccattggaagctactaggtggttaagaagagacttgaacaagtctcctagtcctattgtagg |
| fascicularis | | gcaacactatcaaattgatcgggaactacctgttaaattctatgctaccgcagcctggtcgaaagacgggttgcgcaccaggttaacga |
| | | ccttggggatggtaataatgcttagatagggtatatccgcagcgaaatcctaaggctcaggctatggatagcgttcacagactagacga |
| | | tagtgggcttggcagctctatcttgccacagcttaagatatagtcgaacccttcagagatgcggggcgtgagtgatggatgcaagtctgt |
| | | tgctcagcgcatcagatgctagtagctggttcctgccgaagtttccctcagga |
| Grovesinia_ | 128 | gcgtataggggcgaaagactaatcgaatcatccacatggcatatatttatatagttgctaggtgattaagaagaggcttgaacaaggtct |
| pyramidalis | | cctagtcctaccttagggcaacactatcaaattgttcgggaactacctgttagaattctaagctaccgcagctttgtcgaaagacggttgc |
| | | gcaccaggttaatgacctcggggatggtaataatgcttagatagggtatatccgcagcgaaatcctaaggcttctataagctatggatag |
| | | cgttcacagactagacgatagtggggcttttatgatattgaatcattagggcttaagatatagtcgaaaccttcagagatgaagggcatga |
| | | gcaataggatatgaatacaatccgaatgctcatgcattacatgctagtagctggttcctgccgaagtttccctcaggaatagttgctaggt |
| | | gattaagaagaggcttgaacaaggtctcctagtcctaccttagggcaacactatcaaattgttcgggaactacctgttagaattctaagct |
| | | accgcagctttgtcgaaagacggttgcgcaccaggttaatgacctcggggatggtaataatgcttagatagggtatatccgcagcgaa |
| | | atcctaaggcttctataagctatggatagcgttcacagactagacgatagtggggcttttatgatattgaatcattagggcttaagatatagt |
| | | cgaaaccttcagagatgaagggcatgagcaataggatatgaatacaatccgaatgctcatgcattacatgctagtagctggttcctgcc |
| | | gaagtttccctcagga |
| Myriosclerotinia_ | 129 | gcgtaaaggggcgaaagactaatcgaatcattaacactactaggtgattaagaagaaacttgaacaagtttcctagtcctatttcagggc |
| curreyana | | aacactatcaaattgatcgggaactacctgctgaattctaagctaccgcagccttgtctaaagacgggtgcgcaccaggttaataacctc |
| | | ggggatggtaataatgcttagatagggtacatccgcagcgaaatcctaaggcctttgctatggatagcgttcacagactagacgatagt |
| | | gggcttctcagtgagagcttaagatatagtcgaaaccttcagagatggagggcgtgagtatcttcgaatgctcatgcatcaaatgctagt |
| | | agctggttcctgccgaagtttccctcagga |
| Myriosclerotinia_ | 130 | gcgtaaaggggcgaaagactaatcgaaccattaaggaatagaccaagctctaggtggttgagaaacccctttggtattagtcctggag |
| ciborium | | acagggcgacattgtcaaattgttcggggaccacccgttaaattacatactaccgcaacaggcctgaaaggcctgcgagcactaggg |
| | | gtagcgcctctagggatggtaataacgtatgtatagggtatatccgcagcgaagttctaaggcctcacggctacgaatcgcgttcacag |
| | | actagacggcaatgggctcttcacagggcttaagatatagtcgaacccctcagagatgaggatggaatcaatgctagtagctggttcct |
| | | gccgaagtttccctcaggagcgtataggggcgaaagactaatcgaaccattaaggaatagaccaagctc |
| Myriosclerotinia_ | 131 | gcgtataggggcgaaagactaatcgaaccattaaggaatagaccaagctctaggtggttgagaaacccctttggtattagtcctggaga |
| luztlae | | cagggcgacattgtcaaattgttcggggaccacctgttaaattacatactaccgcaacagcgttgaaaggcctgtgagcactagaggta |
| | | acgcctttagggatggtaataacgtatgtatagggtatatccgcagcgaagttctaaggtcttacggctacgaatcgcgttcacagacta |
| | | gacggcaatgggctcttcacagggcttaagatatagtcgaacccctcagagatgaggatgggatcaatgctagtagctggttcctgcc |
| | | gaagtttccctcagga |
| Myriosclerotinia_ | 132 | gcgtataggggcgaaagactaatcgaaccattaaggaatagaccaagctctaggtggttgagaaacccccatggtattagtcctggag |
| caricisampullacae | | acagggcgacattgtcaaattgttcggggaccacctgtcaagttacacactaccgcaacagtgctgaaaagctgtgagcactagagat |
| | | agcgcctctagggatggtaataacgtgtgtatagggtatatccgcagcgaagttctaaggcctcgcggctacgaatcgcgttcacaga |
| | | ctagacggcaatgggtcctcgcagggcttaagatatagtcgaacccctcagagatgaggatggaatcaatgctagtagctggttcctg |
| | | ccgaagtttccctcagga |
| Botrytis_ | 133 | gcgtataggggcgaaagactaatcgaaccattaaggaatagaccaagctctaggtggttgagaaacccccttggtattagtcctggag |
| tulipae | | acagggcgacattgtcaaattgttcggggaccacctgttaaattacatactaccgcagcagtgctgaaaggcctgtgagcactagaggt |
| | | aacgcctctagggatggtaataacgtatgtatagggtatatccgcagcgaagttctaaggcctcacggctacgaatcgcgttcacagac |
| | | tagacggcaatgggctcctaacggggcttaagatatagtcgaacccctcagagatgaggatggaatcaaagctagtagctggttcctg |
| | | ccgaagtttccctcagga |
| Botrytis_ | 134 | gcgtataggggcgaaagactaatcgaaccattaaggaatagaccaagctctaggtggttgagaaacccctttggtattagtcctggaga |
| porri | | cagggcgacattgtcaaattgttcggggaccacctgttaaattacacgctaccgcaacagtgctgaaaggcctgtgagcactagaggt |
| | | aacgcctctagggatggtaataacgcgtgtatagggtatatccgcagcgaagttctaaggccttcacggctacgaatcgcgttcacaga |
| | | ctagacggcaatgggctcctcgcggggcttaagatatagtcgaacccctcagagatgaggatggaatcaaagctagtagctggttcct |
| | | gccgaagtttccctcagga |
| Sclerotinia_ | 135 | gcgtataggggcgaaagactaatcgaatcattaaggaatagaccaagctctaggtgattgagaaacccctttggtattagtcctggaga |
| sclerotiorum | | cagggcgacattgtcaaattgttcggggaccacctgttaaattatatgctactgcagcagtgctgaaaggcctgtgagcactgagggta |
| | | acgccctcagggatggtaataacgcatatatagggtatatccgcagcgaagttctaaggcttcgagctatgaatcgcgttcacagacta |
| | | gacggcaatgggctcctcgcggggcttaagatatagtcgaacccctcagagatgaggatggaatcaatgctagtagctggttcctgcc |
| | | gaagtttccctcagga |
| Sclerotinia_ | 136 | gcgtataggggcgaaagactaatcgaaccattaaggaatagaccaagctctaggtggttgagaaacccctttgtattagtcctggaaac |
| tetraspora | | agggcgacattgtcaaattgttcggggaccacccgctaaattacatgctaccgcagcagtgctgaaaggcctgtgagcactagaggta |
| | | acgcctctagggatggtaataacgcgtgtatagggtatatccgcagcgaagttctaaggccttctgctacgaatcgcgttcacagacta |
| | | gacggcaatgggctccttgcggggcttaagatatagtcgaacccctcagagatgaggatggaatcaatgctagtagctggttcctgcc |
| | | gaagtttccctcagga |
| Nostoc_sp._ | 137 | agataactgagccttgaaggagaaatccctcaagtggaagctctcaaactcagggaaacctaaatctggtgacagacatggcaatcct |
| PCC73102. | | gagccaagcccgaaaattttagatttgcgattttagatttgggattagttttcaatctaaaatccaaaacccaaaattgagggaaggtgca |
| | | gagacccgacgggagctaccctaacgtaaagtcgagggtaaagggagagtccaattctcaaaatctgatttggctattgccatcaggt |
| | | agcagtgaaaactgcgggaggatg |
| Chloroplast_ | 138 | aattggattgagccttggtatggaaacctactaagtgataattttcaaattcagagaaaccctggaattaataaaaagggcaatcctgagc |
| Gentiana_ | | caaatcctatttttcgaaaaagcaaaaagaaaggcttagaaagaaataaaggataggtgcagagactcaacggaagctgttctaacaa |
| ligustica | | atggagttggttgcgttggtaaagaaatctttctaccaaaaattccaaaaggatgaagaagaggggtgactgtatcaaatgattcgccca |
| | | cagcctgtagatcaacggattaatcggacgagaataaagagagagtcccattctgcatgtcaatgccgacaacaatgaaattgatagta |
| | | agagg |
| Chloroplast_ | 139 | atcggtagacgctacggacttaattggattgagccttggtatggaaacctactaagtgataactttcaaattcagagaaacccgggaatta |
| Draba_ | | acaatgggcaatcctgagccaaatcctggtttacgcgaacaaaccaaagtgtagaaagcgagaaagaagggataggtgcagagact |
| tomentosa | | caatggaagctgttctaacaaatggagttcactaccttgtattgatcaaatgattcacttcatagtctgatagatccttggtggaactgattaa |
| | | tcggacgagaataaagatagagtcccattctacatgtcaatactgacaacaatgaaatttatagtaagatgaaaatccgttgacttttaaaa |
| | | tcgtgagggttcaagtccctctatccccccaaacagtctgtttg |
| Chloroplast_ | 140 | atcggtagacgctacggacttaattggattgagccttggtatggaaacctactaagtgataactttcaaattcagagaaacccgggaatta |
| Draba_ | | acaatgggcaatcctgagccaaatcctggtttacgcgaacaaaccaaagtgtagaaagcgagaaagaagggataggtgcagagact |
| dubia | | caatggaagctgttctaacaaatggagttcactaccttgtattgatcaaatgattcacttcatagtctgatagatccttggtggaactgattaa |
| | | tcggacgagaataaagatagagtcccattctacatgtcaatactgacaacaatgaaatttatagtaagatgaaaatccgttgacttttaaaa |
| | | tcgtgagggttcaagtccctctatccccatccccaactatactc |
| T4 td | 141 | GATGTTTTCTTGGGTTAATTGAGGCCTGAGTATAAGGTGACTTATACTTGTAATCT |
| | | ATCTAAACGGGGAACCTCTCTAGTAGACAATCCCGTGCTAAATTGTAGGACTGGT |
| | | TCTACATAAATGCCTAACGACTATCCCTTTGGGGAGTAGGGTCAAGTGACTCGAA |
| | | ACGATAGACAACTTGCTTTAACAAGTTGGAGATATAGTCTGCTCTGCATGGTGAC |
| | | ATGCAGCTGGATATAATTCCGGGGTAAGATTAACGACCTTATCTGAACATAATGC |
| | | TACCGTTTAATATT |
| Tetrahymena | 142 | TAACTATGACTCTCTAAATTGCAAAATTTACCTTTGGAGGGAAAAGTTATCAGGC |
| thermophila | | CTGCACCTGATAGCTAGTCTTTAAACCAATAGATTGCATCGGTTTAATAGGCAAG |
| | | ACCGTCAAATTGCGGGAAAAGGGTCAACAGCCGTTCAGTACCAAGTCTCAGGGG |
| | | AAACTTTGAGATGGCCTTGCAAAGGATATGGTAATAAGCTGACGGACAGGGTCC |
| | | TAACCACGCAGCCAAGTCCTAAGTCAACATTTCGGTGTTGATATGGATGCAGTTC |
| | | ACAGACTAAATGTCGGTCGGGGAAGAATAGGTATTCTTCTCATAAGATATAGTCG |
| | | GACCTCTCCTTAATGGGAGCTGGCGGATGAAGTGGTGCAACACTAGAGCCGCCG |
| | | GGAACTAATAATAAACATAATATTAGTTTTGGACTAATCGTAAGGTAGCCAAATG |

TABLE 3
Group II introns (flanked by 10 nt exons)

| | SEQ ID | |
|---|---|---|
| Species | NO: | Sequence (10 nt exon - intron - 10 nt exon) |
| Paraburk | 143 | tacgcctatggtgcgacacgaagcatgaggctcgttgttgaagagcgcagtgcatgtccgcttcatcattgtccagtcg |
| holderia | | cgaacatgttcgtacgatagaggacccgctgtgcgaccagaggtggcctaggaaagcatctttcgttggcgacgacg |
| xenovorans | | ctggatgaagcctttccgacaacgtagtccaactgtatgcgccgccgcgaggtgacgtgtagaagctctgagcagcg |
| | | acggggccgagacgcaaggctggccggtatgggtaacacatgtgaatcgctgataaacgtcgtcagaagaagaatg |
| | | ccaaagctgctgtcaggcatttaccaaacaggtgcacgggggatgctccgttcataggtcggagcacgtcgtcgag |
| | | atgcccgccggcgaaaaggcggaccctaacccggtcgtgtgatgaagcgggaacgtggtaagcccatacagttgcc |
| | | cacgaggcagacgagtcgcaaggcgagttgttgactgtgcgggtaagagaagacggaaaaagcgaatgctgggct |
| | | gaaacggtccagataagggtttcaacattaccctacgcgaaagcgtgcagacttccgtccggtgtccagcgcaaagct |
| | | gtcgaaagacttcgtagatggaagcatgaaagagcatcgttcgcggtgtctgcgcccttcgctgcggcggaagaacc |
| | | tcggcaagacaatgcgcaggcgtcagatggtggacgccggtcacaccggcgtgtgactttggaatgcttgagccgta |
| | | tgcagggaaacttgcacgtacggttcttaggggaggccgcagcggcgacgctgtggccttacccgattttaagcccg |
| Methanosarcina | 144 | gagggtcatggtgtgccacgaggctgcagaaaagatgagggtagacccctcggactcgctttacaggaagttattcc |
| acetivorans | | aaaccacccataagctgctttggtcaaaagccacggtagtgagcattatgggaaaaggcgaggctatgacttcgtcag |
| | | gtgtgggttaaggagccgaatgaaaatgaaccactgaagacgtatcgaaaacgcgatgatgtcgtcaaaaccaggtg |
| | | gcgtgactaacctgggataagtctggaagatactctgtttactgaccagatggtgaccggtatataggtggcgggaact |
| | | taactcgtgcttttctgtggaacgtgggaacctgctcatcgatgtaaagagaaacctccaagcggtggacccgcaagg |
| | | acaaaagtatcgatgcgatgaacaggggcggaacaattcgtagtagtgatgaagtgcctgtaatgggcatggagcga |
| | | agggattgtattatccagttttgaactgcggtcaaccttgtcttcaagggaggaaccaatggacaaaacaaggctttatg |
| | | gctggataatgggagccgtatgaatcgagagattcaagtacggatctgagagggcctgggggtgaaattcccctggg |
| | | ctacttaccaatgcccaga |
| Thermosyn- | 145 | agacaaggcagtgcgacgcgaaagctagccagatgattgtcccactagcccaacaagctagaacgggaccggttgt |
| echococcus | | tcccccaaccgtagcctaaggaggcatgcgtgactggtaacggtcaggtatgaagccctcccgacaatgaagcccg |
| elongatus | | aaccggaaggttggagccgaatccgtgaggaggaagcaacttcaccagcgtcaggtgatagggagctaggcttgag |
| | | ggtatggtgaacgcaagtgaagtgacgctagaagcctcgttactccaagcaggccaaagatgctgataggcctgagc |
| | | caaaatggcaaagcggattggattcactgcttgcttttcagtgaacggggacagcaactccgccggggtatagtcacc |
| | | acctaacccctcgtgtcatctggttggaacgcggtaagcccgtatcttcgccttgaacattcaaggcaggcaaaccgta |
| | | aggaatgctgatgggggtgcgggtagaggaggtgggaaaaagcgaatgctagcctgtaatgggctagatagggatt |
| | | gagaatgctggcaggacattcaacatcatcccactcgaaagagggcagacttcccgccggtccccccttacgagaaa |
| | | gtctatagaaccttcctaatggtgacaatgcaaatggcggtgatcttgagtcactggtgcggtcaccaacctgctcaaaa |
| | | ggtagagaggctgtagatgagtatcgtaaaggttgcatgcacaaccggttcctcttaaatgaggggtcctggggggct |
| | | cgagccggatgcggggaaacttgcacgtccggttcctagggggctagggggcagcgatgcccccctgctacccga |
| | | caatgacggtg |
| Bryopsis | 146 | aatgcatgacgtgcgacacgaaagtcgctgtatttagtagatatttacaaatcttatcctagcattcggatattcccatactt |
| maxima | | tgggtgcaatcctataataataaaagattgtgctaagccttagtaaaagccaaaacttatgatgttctcgtaatataaggca |
| | | agggaaaatagaatagagttggtttaacggcctcgtaaagatgagaacagcccaaaagggttaactacttcgctaaca |
| | | gactgaatggatcgtcgtggtcattcaggaatgcttctaagaagtcggggtgattgtctccaaaactattctatgtatctaa |
| | | atatagtggaacgtgggaaatccaaaaatctccctatgggtaggtgtttagcaataaacactaaaagattttggattgcg |
| | | gattgtcctgttatgtaagaattccatgcaaaggaataatgaacccatagcaaaaaggaaaatatgggatgcgaattact |
| | | cagtaaaaagtaagttctaatacagaaaacagcccatatataactggaattcaggaaagttatttccaacaaatgaggac |
| | | aaggtcttatttcataaaatttatattaaaataacaaagaacaaaggagattgacttatgatagtactttgggagactcacc |
| | | agagtggataccatccaattggaacaccacaatggcacaagttcggaatttaaaatatagaatctttatggcgaaactag |
| | | aaggtaatcggagaacattacgaagattacaaaaattgatgttagatagtcgccacaagatactcctgtccgtgagaag |
| | | ggtaacggtgattaatgatggaaaaatagattcctgggatagataaagttctaataaaagataaaaatgacagaatggta |
| | | ttggtagaaaaactctataaaatgaatattttacaatacgagcctaagcccgtgaagagaatacaaataccaaaatcaaa |
| | | cgggaaaactagacctctgggaatccctaccatactagacagaatgtatacaagccatagtgctaaatgcattgcaacc |
| | | agaatgggaagcctgttttgaagcaaatagttacgctttcgcccaggaagaagtgcacacgacgcgattggtcgggtg |
| | | ttcaacgtactaagtgttaagtcaaatggaaacaataataaaccatggatattagatgcacatatcgaagggtttttcgata |
| | | acatatgtcatcaagacatattagaaaaactcgacaacttcccagccaaaaagcttataaaccgttggttaaaagcagg |
| | | ctacatggaagaagatatcttttcaaaaactgagaaaggtacaccgaaaggatccattatcagcccactactcgcaaac |
| | | atcgcattgtatgagttagaggaatttcttaaagcatcaccagactcaacaggtcgagtgagggggaatagagtatacg |
| | | ttcgatatgcagacgacttcatagtcatatgcgcatcaaaaacggaagctcaacaaaccaagaaacaaatctcaggttg |
| | | gttaaaaaccaaaggtctcagatttgcgccaaataaaataaaaatcagtgatattgataaaggctttgaatttttaggtgta |
| | | gatattcgccatgtaagtacgaatgcctcgcataaaaccaaaggtaaaatgcttctgatacgcccctctacaaaggctat |
| | | cgacaaaataacacaaaaactgaaaaaagaatggtttctcccgagaagtaaactcatagaaacggtactcacacgtct |
| | | aaacccaatgatcacgggctgggcaaattaccatcaaaaattttcatccacggatatctttaataaattagacaactggac |
| | | attccagaaaagttggaattatgcatccagaatgcacgccaacaaatcgaaacattggatttatgataagtattatggga |
| | | acttcaatttaaaccgaaaggaccgttggatattcggagataaggaaacaggaagataccttaaaaagtttgcttggttc |
| | | aatataaagagacacgtggtggtaaaaggctacaattccccttgtgacccaaggttacaagaatactgggaagaacgc |
| | | cggaaaactctctataaagtcagtagaacctaatcggaacaaataatcgcgaaaaatcaagattatagatgtccagtttg |
| | | tgaacacccgatgagaatctacagaaacatcacctaatttcccaacgaaatataaaaattgaaaattattggaacctagt |
| | | atatatcatagacaatgccatcaattctgacattccaacccgaaaattgaacgagaattacatggaaaattttattcgagg |
| | | aaaatcgtgttcgtaagcgtctaacaaagaatccgcgaacacacatagaaagattaaaactcgaacttttagaacataa |
| | | ggcttgagccgtatgcggggagactcgcacgtacggttcttaggggggaattggtggaaacgcctgacctacccgac |
| | | tatttaactg |
| Methanosarcinales | 147 | cgttactcacgtgcgccacgaagctgcacaagagatgagggttcggcccctcgaagcctgcttgcaggagccggctt |
| archaeon | | caaaccgccttaagctgctgtggtcaaaagccatggtggtgagcgttaaggaaaaggtcccacaagatgggatcagg |
| | | tgtagatcatggagacgaacgtaagtgaaccgctgaggaagtgtcgaaagcgtagggacgacgtcaaaaccaggg |
| | | gctggtcgttgacctgggataagtctgggagagcgcctgcttactgcccagatggcgtccggcatgaaggcggcgtg |
| | | aacatggtcctggctcttgtgtggaacgtgggaacctatcccctaatgttaagggagaaacacaagtgggaggacccc |
| | | acaagtgtgagagtaccgatgcaggggaataggggcggaccagcccgtattagtgatgaagtttctgtaatggagata |
| | | gagcgaaggggttgggtcatcctgcttgatttaaagatcaaccaggagatgggaggaatctttgatgaaagcaaagcc |
| | | atttaagatttccaagaaagttgtattggaagcgtggaaagaggtgaaagcaaaccgtggagcagcaggtgtagataa |
| | | gaagtcgattgcggattttgagaaggatcttaataataacctttacaagatcaggaatcggatgtcatcggggagctactt |
| | | tcctccaccggtgaggacggtagggataccgaagaagagtgggggagagaggttgttaggcatacccactgtggct |
| | | gacagagtggcccagacagtggcaaagatgtaccttgagcctttggtagagccatactttcacaaggactcatacggg |
| | | tacagacctggtaaatcagcgattcaagcagttggagtgacgcgtaagcgttgctggaggtatgattggatgcttgaatt |
| | | cgacatcaaggggttatttgacaatattaatcataacctcttgataagggcggtaaggaaacacacaaactgtaaatgga |
| | | tgcttttgtacatagacagatggttgaaggctccatttcagagacaagatggtacgttagtgcagagagaaaaaggaac |
| | | tccccaaggtggcgtgataagccccttgcttgccaatctcgttctgcactatgtgtttgataaatggatggagcggaacta |
| | | tccgcaagttccattctgccgctatgctgatgatggtgttgtgcactgccggtctgaagccgaggcattaaagctaagga |
| | | aaactctgggggctaggtttgggaagtacaatcttgagcttcatccggagaagacgaagatcgtctattgtaaagatgat |
| | | gatcgtcgagatgactatcccaacacgagttttgattttcttggctttacattccggccaaggaggtcaaagaatcggtgg |
| | | ggtaagcttttcatcaactttactcctgccgtgagtaataatgcggcagaggcgatgaggcaaaaggctcgaagatgg |
| | | aagatgcacctgcgaagtgacaagtccttggacgatctgtcaaggatgttcagtcctattatcagaggctggatcaatta |
| | | ctatgggcgcttttataaatccgcgctgtacccaactttgcaacatctgaacagaacgttagtcagatgggcaatgagga |
| | | aatttaagaggtttagacaccatcgtaggcgtgcggaatactggttaggggaaattgctcaaaggcagcccgggctgtt |
| | | tccgcattggcagatgggtgtgagacctacggctggatgatgggagcccggtgagctgagaggttcacgccgggttc |
| | | tgagaggacgtaggggtgagattcccctgcgttactcacctgcgccacga |
| Marchantia | 148 | aagagttatcatgacaaaatgcaattacgagcaactactagaccccgagatatttaggctagcttacgagctaaagaaa |
| polymorpha | | tcgaaatcaggcaatatgaaacctggtgcggataaagaaactctcgacggtttctcccaagcctatgttgagaaggtcg |
| | | tccgtcaactaaaagacgaatcatttcaattccgtccgtcacgaagagaattcattcctaaagcagacggcaagcttcgt |
| | | tccctgggcataccatcacctagagacaagatagtacaagaggtcatgaggaggatccttgaacctgtatttgaaccgc |
| | | gattcctggattcgtctcacggatttagacctcatcgatcaccgcacacggctctacgacaaatccgtcgatggacagg |
| | | cacctcctggatgatcgaaggagacatcaaaggatacttcgacaacatcgatcatcacctactcgcgggattcatagca |
| | | gagttggtaaaggaccaacggcttctcgcgctttattggaaattggtacgcgctggctatgtaaatcaaggcaaagcag |
| | | agccacacttgctaacaggagtacctcaaggaaggatactatcgcctctgctttccaacatctacctacaccagttcgat |
| | | ctattcatggaggaaatcaaagtcaaatatacaacgaccggtgcgctttccaaaaacaacccgatttacttgaaggcgc |
| | | ggaataaatactacaaacttgtgaaatcattaaaggcttcttccgccgaaatcatccgagcgagacgcgatatgttgaaa |
| | | atgacttacgggattcaaacaggttctagggtgcgttatgttagatacgcggacgattgggtgatcggggtcacgggtc |
| | | caaaagccctggccgtacaaatcaaagaagaggtctctaccttcctccaagaaaaactaaaactttcgcttcaggccga |
| | | aaaaacacgtattaccaatctatcaagaagcgaagctttattcctaggaaccttaataagcataacaactcgtaaatacgt |
| | | gcaaagccagaaggtgggggggggcacaggcgagcctccctcggtagaatacgcctgtgcatccccatcgatatc |
| | | cttatcgggaagctctcacaaatgggggcgtgcgacgaaaagggaacgcccaaagcggtgaccaaatggatctttct |
| | | aaacgtgggagaaataatcaacaaatatatggctgtgttccggggatactacaactactactcattcgcagacgatatcc |
| | | atcaccttctccaaataatatacatactaagatactcggctatcaacacggtcgcccgtaaactggggcttaacacagcc |
| | | aaagtaataaaacgcttcggcgtggacctaatcttccgggaccacacgaatgagatcaagcataagctcaatttcccgc |
| | | gatccctacctaataagcgcatgaacttcgccttgagtccgccttcggaccctagagttctatttgatacaagctgcgctc |
| | | gcattcagtgttgaacgacctgtg |
| Vibrio | 149 | ttttcttttggtgcgccgagcatggcgttgatctaggaggtgaaagtcctctacgggctcagtcgagcgagaaccgttag |
| harveyi | | cctatgcaagggtgttcactgtgaggtgaaatctgaaggaagcaaatggcaaaacttggttttgacgaacagaaatctg |
| | | atagtaggtcactacaacttgggcaacctagccatagatagaatagcccaatgcctcgacgggaagtgtgtagtgata |
| | | aatcaggcacggccaagggaaagagcaacgtcttacctcgggagatcttattgtttgtctcgatagagactaatgcagc |
| | | agtgatgttgtgtgacgggcaataagaagtcagcagagggcatagtaccttgagacagtacaacttaagggaaggcc |
| | | tgaactgaatatatcaagaagtaagtcactagacactcaatcatgtggagtcatagcaagatggacatcatctctacgtc |
| | | ccaatgggcgataccgcaagtaaagctcacggtgacgaagaatggcaagcatggtcggcgtagacaggaggacga |
| | | gtcttggtgacctcaacttcgttgatggaacaaatctgctcctcaaccaacctgaatcaagctttacgaagagtgaagaa |
| | | gaacaagggatgtgcaggtgtcgacaaactcgacatagatgcaactatctttaagcttcgacaagcttcaaacgggca |
| | | agcgcttcgccagagtcttctggatggaagctaccgacctcaacccgttcttggtgtgggaatccccaaaccgagtggt |
| | | ggcgtcagacaactaggtatcccaacagtgctcgatagaattgtccaacaggcaatcacctcagtactgtcagatatct |
| | | atgaagctaagttctccaatagcagttacggatttagacccagccgcagtgcacatcatgcgctagcggcagccagtc |
| | | gctacatccgagaggggcgaggttatgtagtagatatcgacctagcgaaatacttcgataccgttaatcacgatagact |
| | | gatgcacagactgtcagaggatatcgcggataaacgagtattgaagctcatcaggtcatatctacaggcaggcataat |
| | | gcgaaacggtttagtcgaacagagacaacgagggacaccacagggtggtccattatctccgttgctatccaatatcgt |
| | | attagatgaactggataaagagctagagcgtagagggcataagttctgtcgatatgcagacgactgtcaaatctatgttg |
| | | gtagtgaggaagccgcttatcgagtgaaagagtcgataacggagtacttggagcaaaaactgaaactcacggtaaac |
| | | cgagagaaaagtgcagcaacaagagtgacagagcgaacttacctaagccaccgcttcgggatagatgggacaatcc |
| | | atatatcgaagccagcgcaaactcaaatgaagacgcgagtgcggcagataacgaagcgaaaccgaggtcgagagtt |
| | | aaaggtgataatcgccgagttaactcaatacctaagaggttggcaagactacttcaaactcgccatacggaagagcag |
| | | catgaagcgcttggacgaatggatacggcgacggctaagatgctatcgacttaagcaacgcaaacacagatacggc |
| | | atagcgacatggttacagcgacaaggtgtaacagagcgtaatgcgtggaagttagcgatgtcagataaaggttggtgg |
| | | cggctagcattaacgccccagacaaatcacgctatgccaacaaaatggttcgaggagttgggtctgtactcgttgcga |
| | | gatgggtatgagtcactaaaagtatattcggaaccgccgtatgcgacccacgcttgtacggtggtgtgagaggacgga |
| | | ggccgtgaggcctcctcctactcgattattgccttt |
| Crocosphaera | 150 | ttaggggatagtgcgattcgttgagctagaaagctgacatccaagtatattacaaggatacagcgtgtagctcgttcaag |
| watsonii | | agccttttaaaaggtaatccaaaggttaaatgaccaatcaccctccgaagaatcaccctccgaagagaaaacaaagcc |
| | | tagagcgcattctttaaaaggggtaaatacgaaggtcgaatgggtaaaacaacacccaacaaagcaataactaataaa |
| | | gtttacataacttaattagttattaccgaactagctaagtagggataaccgaacagccaatgaacctggaaacttgaataa |
| | | ctgttggtttgttggcagaaaggagccacactcggaatcaagggataactcatcccaagagtgacgacgtaaccctcc |
| | | gttttggaattagtaaacccaggatggaagcagggttaaaaggagaacccgtcatgaaacagcatttcggagtcgttct |
| | | ttaagtcaatcggtcatggttaaacattacgttgaatcatcgtcctgaaccctcatgaatactataagtagccaaatctgttt |
| | | gataggctgcgttcaaaagaacaaggtgaaaagtggagagtttgtcccctcatccacacagattcctaaaaataacctg |
| | | ggggagagatgaaacctaaaaccgatatacccaaatcaacggaacgaattaagtccatgatttctcttaccatcaggtc |
| | | gatgatgtaagtagtgacaaacttaagcgcaagggtcatggatgtgagaagcaataagaagcaaatgcctacttgtaat |
| | | gagtacggataagctgacgtattgctggttaattagaagaaatagtctctattagaggtagttaaatgtcgaatgcgagta |
| | | taaccaagactacgccagaatggaacacaatcaactgggcaaaagtccagaggaaagtgtttaagcttcaaaaaaga |
| | | atatttcaagctgttaaatcaggtaataaagttaaagctaaaaagcttcagaagttactgttaaagtcacactacgcaaag |
| | | ctcctagccatacgcaaagtgacccaagacaaccaaggcaagaaaactgctggagttgatgggataaaggcattacg |
| | | ccccaaccaaagactaaagctaattaaagaacttaccttaaaaggttacaaagcaaaggcacttcgacgggtttggatt |
| | | cctaagcctggaagagaagaaaaacgtggactgggaataccaaccatgaaagacagagcgatgcaagccttggtta |
| | | aatcagccttagaaccatattgggaagcccaattcgagggaacatcttatggtttccgtccaggcagaagcgcacatga |
| | | cgctatcggtagaatatactctggaatcaatcaatgttccggtggtaaatatgtcttagatgccgatatagcaaaatgcttc |
| | | gataagattaaccatgagtacctactgtccaaagtagattgtccatatctcatcaaaagaatcattaaacaatggttacaa |
| | | agcggggtaatggacaatggtgtatttgaggaaaccgattcaggaacacctcaaggtggtgtaataagtcccttgttgg |
| | | ctaacattgcactacatggaatgataaatgacacaatgaacaagtttcccaaatccttttacagaaatgggaaacaagtg |
| | | agggataatcaacccaaaatcatccgatacgcagatgacttcgtaattatacataacgaatacgaagtcatcctacaatg |
| | | taaagaaataattgcacaatggttaaaaaaggctggactggaactaaaaccagaaaagacctccatcagacatacctta |
| | | aaaagtattgaacacgatggacaaaccgttgaccctggtttcgattttcttggattcaacatcaggtcatattcaaaggga |
| | | aagcaccgttcaggtaaaaccccacgtggcaagataataggaagcaagacactaatcaaaccaagcaagaaaaaaa |
| | | tactagctcaccacgaggctttaaaagaggtcattaaagacaataaaaaagccccacaagcagcattaatcgcaagatt |
| | | aaatcccataatactaggatggtgtaactactatcgaacggtcgcaagtaaggaaacatttgcatccgaaaacaacgta |
| | | ctatggaatatgctaagagcttggacagttaacaggaaaaagaaaaaaacaccattgtatgaagcccttagaaaatattt |
| | | ctcatacggaaagtacggaaaatggacgtttcaaacagaagaagcagtcctctactatcatgccgaaacggaaataaa |
| | | gagacatcaattggttaaacctgacgcatcaccttatgacggaaactggacttactggagtaaaagacggggaacatat |
| | | accgggacaccagctagagtatcaagactcttaaagaaacaaaaaggtatatgccctcaatgtaagcaacacttcaca |
| | | cctgaagacttgatagaagttgaccacattatccctaaatcgaaaggtggtttggatacatacaacaatcttcaagctcta |
| | | catcgtcactgtcatgaggccaaaagcaaaaatgactacctctatgattggcacttataatggctatgaatggaaagacg |
| | | atgtattaacagtaccctagacaagggactaattgccgagagccgtgtgaggtgaaagtctcacgcacggttcggact |
| | | gggagtcgtggtaggtgactaccacttcgacccctaatcaagcttatt |
| Candidatus | 151 | cgttaattcggtacgccaagaagcgtatatatatcagttggtgcaaatccaacccgccaattgaccgttcgggcggtagt |
| Kuenenia | | gaaagaggcagaaaacctcacgggagaacgtaaacagcgatgaatacgttcgagtcccctgtagcgagcagtcaga |
| stuttgartiensis | | ccgtagcgcaagcgaacacgcaggcttcgttacgctaaaaatacgatagatgagcctcccggattgggcgaaatctgt |
| | | aatcgcataggtagaaagagacaaacggtagaactatgcggtgtcgtcggagttgagagtgacggtatgactgtaaa |
| | | aggtatatatatgaacttgggaggtctcattgttcaggttgatgaccgtaacgcatactataagggaagccgaagggtat |
| | | agcgggataatgagaagtcggagatgcccatagtagtgaagatggtgaagacaacataacttcaccgtagcgaagg |
| | | ggcattacttcagttaacgtttacaaagaaaggaggaaattgtattgggagacctacaagcaataccgaaatcgcaaat |
| | | gagcgatgatgaaagagtccgagactttcagcgtaaactatatcgaaaagccaaacaggaagaaggatttcgcttttat |
| | | gtattgtatgacaaggtgagaatgttgcactttttgagagaggcgtacaagcgctgcaaagccaatggaggaagcgcc |
| | | ggggcagacgggattacatttgaagatgtggagtcatacggggtggagaagtttcttggagagataatagaggaactt |
| | | gaaaacaaaacctacgaaccacagccggtactgagagtgtacatacctaaaaccaacggtaagacacgaccattgg |
| | | gaatccctgtgataaaagacagggtagtgcagatgagtgtaaagcttgtaatagagccgatattcgaagcagactttga |
| | | agatagctcctacggattccgaccgggacgttcagcgggcgatgctgtgaggaaaatcaaagaaaaactgcgagaa |
| | | gggaaaacagaggtatttgacgcagacctgagttcttattttgacacaataccgcacaaggaattgttactgttaatagg |
| | | gatgaggataagcgacaagaacgtactgcacttgataaaaatgtggttaaaagcaccggtgatagaggaaggcaaac |
| | | caggaggcgggaggaaaaataagataggaacaccgcagggaagcgtaatatcaccattattagcaaacatatatctg |
| | | cacatgctcgacaaagcagtcaatagagagaatggggttttctataagtatgggataacaatcatacgttatgcggatga |
| | | ttgggtattgatggcaaagcgaatacctcgtgaagcattagactatctcaacaggttgttaaagaagttaaagctgagtct |
| | | taacgaggataagagtaaaatagtaaaagctgaagaagagagctttgactttctcggacataccatatccttctcagaag |
| | | atttgtttggtaggaaacacaagaaatactggaacatagaaccaagcaggaaatcacagaagaaggttagggaaaag |
| | | ataggaaactacctaaagagcaatgggcataaagcggcggaaaaagtagctaatgaactgaatgcaataacaagag |
| | | gatggataaattacttcacaataaagggagtaacgtatccaaacaaggcgaaaagagacctacgatattatcttttcagg |
| | | agattaacgagatattataagaggaaaagtcagcgcaggagcaagctctataatcgaggagcgtttaaggaattagtc |
| | | aatagatattgactaatagacccgacaaaatatgttcctgccagacaacctgtgaaagcttaagaagaagactgtcgga |
| | | aagccgtgtgcgggaaaattgcatgcacggtttgacgaggggggcagatgataagtatatggcggagatatggtggc |
| | | actggcgggaaaccagccagcaaacagagaaaacaaacttctgcctaagttctagtcgtctgtttctactctaccgttca |
| | | ttag |
| Candidatus | 152 | agcgccgggtgtgcgcccagcatgggcgcgatgtgatggggtggaagtcccctgtgggagtaccgacgtgacttca |
| Accumulibacter | | tgatgaagccatgataaccactagccgaaggcaagggcaaggtcgtgaggacttgtctggaggaagccggagcgc |
| phosphatis | | aaaagtgcgaggcgacggacagaaactgcatagaaggcctggtctgcgggcgagttggcaccccatgacgaagcc |
| | | caccggatacggcggacaaggtaaatgcagcgcttgtgcacggaaacatcgcgttcttacctggggagacctgatcg |
| | | gcaagcgattgccgacgtttcgcaaaagcagtggggatcgctgcttaacagaatgatccgggcgttgcgggaggctg |
| | | ccggccgcagacgagcgaacaggcagcgagcgaagcgaaggccatagcgcgacggcgagcaatcgccagcgt |
| | | gactgatcaggagtcagcagaggccatagtagcgacaccgaaaggtaacgcgaagggccgaacatgaacagaca |
| | | aggaggagccgtgagtcactcgaacccgacaatgaacccgaccgggggagccggggatcggcgtgatcttgccca |
| | | gccagccttgcacgacgccccgaaggaaggttgtatggggaaggacttgatggaagccgtactctcaccggccaac |
| | | ctgaaacaggcgtggcgtcgagtgaaatccaatcgcggcgcaccgggaatcgacggactgcgcattgaggacttcc |
| | | cggcgtatgcttgtgagcactggccggccatccgccagaccctgagcgagggtcgctatcagccacaagcggtgcg |
| | | acgggtgattatcccgaagcccaatggtggcgaacgcgcgctgggcatcccgaccgttgttgatcgggtcgtccagc |
| | | aagccatcgctcaaatcatgacaccgatcttcgatccggaattctccgaatcgagctacgggtttcggccccgacgctc |
| | | cgcccacggcgcactcaagcaggtcagagccgacctcaaggccggctatcgcatcgccgttgatctcgacttggcg |
| | | aaatttttcgacaacgtcgatcacgacatcctgatggcccgagtggccagaaaggtgagcgacaagcggttgctggc |
| | | cctcatcggccgctacctgcggggggtgtcatgattggttccacgcttcaacccagcgagttgggaacgccgcaag |
| | | gcggacccctctcgccgctgctggccaacatcctgcttgacgatctggatcggactctggaagggcgaggccaccg |
| | | ctttgcgcggtacgccgacgacctcatggttctggtcaagagcgaacgggccggccagcgcgtcaaggccagtctc |
| | | accgcctacctcggccggcaactcaaactgccggtcaacgagaagaagagccaggtggcaaagatcgagcagtgc |
| | | gtttttctgggcttcaccttcaggaagaacaaactgcgctggtcggatgccgccttcgccgacttcaagcaccgcctcc |
| | | gggaactgacggggcgaagctggggagtctcgatgccacatcggttcgagaaactcgggcagtacctgcgcggct |
| | | ggatgggctactttggcatctcggagtattaccgaccgattcccgaactggatgaatggctacgccgacgtgtgcgcat |
| | | gtgctactggaaacagtggcgtttgtgtcgcacgaagatcagccaccttctggcgctgggggtcgaccggcggacgg |
| | | cgatcttgacgggcgtcagcagcaagagctactggcatttgtctcggtcaaaggccacgcaagtcgggatgacgaac |
| | | gattggttgagagcacaaggtctggtgagtatccggaatctttggatgaaggctcacggttacgcttaacaacgtcgtgt |
| | | gcgccctgttgatgaaccgcccggtgcggacccgcatgccgggtggtgtggggagggccggttagacgccggccc |
| | | ttacccgattagggcctga |
| Bordetella | 153 | ctgcggcaaggtgcgccgtatcggcgcttagaagaagcgtagcgcaagctaccagggctactggaatgccctgtact |
| petrii | | caccggacttatccctgcctgcaagggcttgaattccgaaaggaaggggggagtgcagcatgttcggaaaaggcctt |
| | | ttacccaacagctattagcggggggggccagagtaagacgtgtatggtcaaagctgaattgcctgaagtctagttcc |
| | | aagcggtctaccagtcccgacgctggcgaaagtagctgaaacgccagactcgcgagttgtgggaaatgttttgcctct |
| | | cccacaaaccagtatcgcgagcgcaaccccgatcaggggattacggaacgaacggacgacctaaccacgtcgcat |
| | | ctttgtctaagcgtaaatgcgggatcgcctaaacaggtcgtaagacctgcatggcgacggagtcttcgtattacccgat |
| | | gtggcctctgtaatggaggttgctggccgcagcagtaagcggcaccgacatggctataagcgagcagcaatgcgcg |
| | | tgaaaggcccactgtccggggggaaggaagacagcttatctgtaaccaactcatcgaaaaggaggttgtgctgatgct |
| | | aaccgacactactaatcgaaggttggatgcgctgggaatcttgtctcaacagggcaagcgcatcaacggccttagtcg |
| | | tctcatggaaaacccaatcctgtggaagcaggcgtatgtcaacatctatgcgaattccggtgcaacaacggccggcgt |
| | | agatggctcgtcactggatggtatgtcctacgagcggctggccggcctgatggccgccgtcaaaagcggtaattatcg |
| | | cttcaagcccgtgaggcgagtcctcattccaaagagcaatgggaaaacccgtccgctcggcatccccactggggatg |
| | | acaagctggtacaggaggttgtacggatgcttctcgtgaagatctacgagccggtcttttccgacgactcgcacggtttt |
| | | cgcaacgggcgttcgtgccacaccgctctcatgcaagtccggcagaaatggacgggcatgaagtggatcgtgaaca |
| | | tggatatcaaaggttacttcgacaacatcgaccatgaagttctggtggatgtgcttgccaaacgcattgacgataagcgc |
| | | tttcttgggctgattcattcgatgctgaaggcgggctacatggaggactggaagttccatgacaccttcagcgggacac |
| | | cgcaaggcggagttgtctccccggtcttggccaacatctacttgcacgaactcgacgagtacgttgcagggttgaagg |
| | | ccgaattcaaccgaggaaaccgccgggcgtcgaaccgcgagtacaagcggatcagtggtgcaattgaacggttgat |
| | | gaaacgcattgatgcctacaaagccgatggcgactcgccaaaggtcgaggaagcgaagcgtgaattggccgaacttt |
| | | atctacgccgcaaggcactgagcagctccgacccgatggacgccaattaccggcgtctggtgtatgtccggtatgcc |
| | | gatgattttctgatcggcatcatcggcagtcgggacgaggcggtaaccgtcatgcagcgagtcgcaggtttcatcagc |
| | | gacaaactgcacttggagatcgctgaggaaaagtccggtgtggttcacgcatcggacggagttcgctttctggggtac |
| | | gacgttcgcacgtactccggtgatcgcaacgtgcgcaccgtccgttcaggacggagcatcacagcacgcagcgtatc |
| | | cgagcggatgcaattgcacgtaccggctgaaaagctgcgtagtttctgccagcgaaagggctacggcgtctatgacg |
| | | ctcacacccccacgcacaggagcgaactgatctcactgagcgaagcggaaatcgttcaaacctacaacgctgaaatg |
| | | cgaggccttgccaactactacggtttggcgaacagcgtcagtcgtgtcctgaacaagctccatcacctctggttgacga |
| | | gtttgtggaagacattggcggcaaaacgtcgttcgaccgtcacgaaggtagcgcggagcatgaagcgccagggtgg |
| | | atacgtactggctgttcggggtaactcggactcccacacttttcccatctacagcctgaaagacataaggaaagaaccc |
| | | atcacgttccagagtattgacctaccccccaagacttgggttttcacgcatagccgctcggagttgatccagcggctcg |
| | | aagcgaggaagtgcgagtactgcgggaccaccgaaggcagtttccaagtgcatcacattcggaagctgaaggacgt |
| | | ggacaaagggaagtacctctggccacaggtgatgtcatggcgccgtcgcaagactttggtgctgtgcgttccttgccat |
| | | cagaagctacacgcaggcgttctttgaaaggactgttcctagtcaagtaagtggagagccggatacgctgaaaggtgt |
| | | aagtccggttcggaggggggctgacgggtgatcctttacgggacgcccgcagcctaccctacacgaccgtcg |
| Cupriavidus | 154 | agtacgattagtgcgccaggagactggcagagagtagtcgatgaaagttcgatacaggaaaggaatagcgaaccac |
| taiwanensis | | cctggccccgagtcatgcggcggcacccgcgagggtgcagccgaagcgttgacaggggagagtgcaggccagc |
| | | cattgagccgcgaaatcattctgtctggagcgccgacgctgttgatcgaagcggaaggcaacatcgtcgagggcgct |
| | | aaacgcgagttctcggcgggctccacgcggtcgaagaccctgagcatgcacagaagctctctgcacaggaactggg |
| | | agatctcgccagtgcccaccgagcagctggcggtggggggtcggggaaggccgatggccgtacgcccgacatc |
| | | gacgctggcgagaagtcagatgcttgcgtagtaccgaggaacgatccgaacaacgacgcggccgcaaagccggc |
| | | gtctgcggaggatcgggagggaaggcgagcagccaagaggaacgccgaacaatctcccgcgccccggacacag |
| | | agccggtcccgcgcgtcgacggggttgaacggcgtacgcgaagcagcctgtgcccaaaaggcatcgggcaagaa |
| | | ggtgcagttcaccgcgctgatgcaccacatcacgccgcgattgctgatcgacagcttcatgcatttgaagaagtcggc |
| | | ggcggccggggttgatggagtgacatggcacgactacgaggaatgcttggtcgagcggataggcaagctctggga |
| | | cgcggtgcaagctggccgctaccgtgccctgccatcaaggcgagtgtacatacccaaagcggacggcaaacagcg |
| | | tccgctaggcatcgctgcgctggaggacaagatcgtccagcaagcggtggtgacagtgctcacaccaatctacgagt |
| | | ccgacttccttgggttttcctacgggttccggcccggacgtggccagcaccaagcgcttgatgcgctgtgggtggggc |
| | | tgcattggaagaaagtgaactgggtgctcgacgcagatattcgctcgttcttcgacacggtcgaccacgggtggatgat |
| | | gcggttccttgagcaccgaattgcggataagcgcctgctgcggctcatccgcaagtggctgacggccggcgtcatcg |
| | | agaacggggcgaagacggaaatacgggtaggtaccccacagggggcggtgatctcaccgctgctcgcgaacatct |
| | | acctgcactacgtctttgacttatggctccagcgatggcgtcgccgcgacgctaagggtgacgtgatcgtcgtgcgata |
| | | tgctgacgatagcgttgtgggcttcgaggccgaggctgatgcctcgcgattcctggaggccttaaaggcacgatttgct |
| | | cagttcgggctgtcgctcaacgaacagaagacgcgagtgttgcagtttggtcgctacgcggccagcctacgcaagcg |
| | | cgctggcttgggccggccgcagacgtttgactttcttgggttcacgcacatttgcgcgaccaagcggtcgaatggcgg |
| | | tttcatcgtcaggcggttgacatcgagcaagcggatgcgtgccacactcaaggcactgcggcaggcgctgtaccggc |
| | | gccggcacgaaccgatcgcagtggtcggcacatggctacgccgtgtgatgcagggctacttcaactatcacgccgtc |
| | | ccgggcaacaacctgcgattaagtagattccgttctggggtctgccgagcctggttacatgccctccggcgacgtggg |
| | | caatatgggcgaatgtcctgggcgcgattcctccgacgggtcgcgccatatgtaccgtcagttcgcgttctacaccctt |
| | | acccaacacagcgcttcttcgcgtcatgactcaaggcaggagccgtatgcggtagccctgcacgtacggatctgtgc |
| | | ggggggggggggtaacctccgtccctaccgcgatccccgtcaca |
| Clostridium | 155 | gaatatactatagccatacaataaaagtgcgaaacgttatcctataagtaagaaagttttaaaattttcttacgaaaaggat |
| tetani | | agaacttaaaagttctaactgttctactaaagtaataagtgaaaatcttatttaaagcaaacaaccaagtagctttaagtcta |
| | | agtcccctacacaagttttatactactatgcaaaacttgtgaagctaggtaaggtcgtaatccgtgaaagtcggatgcgg |
| | | ggctccttaaaagattactatggtaaacataagctaatccattaagatgcgatttatatgtattttatactgttaaatatttttgt |
| | | gcttgtggcttggtataaaacagttaagatgaagtacttaactggttttggaataattggttgttaaactaaaacattataaat |
| | | cgttagtggatacctaaggtaatcaaaaatagggataggtagaatggaacgtttgatgctgtatatgaagaggtttagta |
| | | gaacctaggacacatatacgggctcagcaggttcatagtagctatgatactcagccggaagtcaaattaattttgaaata |
| | | cttctatggtaacataggagaaggataaaactgagtgagccaaggaacctagtcggtaatagaaaagtggaagttaaa |
| | | acaaatataagattttagaattaatttaattaatgaacggaattaatttaatgatatttaaagttagacggttataaattaaaca |
| | | tttcaaaattaaaccatatccaaattcataaatatagctagatcatatcactagtttaaaaataaataaatcatttcaaattact |
| | | attaagtaaggtattaataccttacttaatagtaatctcattacataagagaattactagattagcagacagattcataaaaa |
| | | ctatatcaactaggacaatagaaaatatatttatacacttcctattatcgagcgaacgccttatgcgatgaaagtcgcacgt |
| | | agggtgtagaccaagcgaaatcctatgcatttaggatagtgaggtatagcaaaggagaaaa |
| Thermosyn- | 156 | aggttcttctgttgcgacgtggagctaaccagatgattgtctcgctaacccgaaaggttagaatgagaccagctgttccc |
| echococcus | | ccagctgtagcctagggaggcatgcgtgactggcaacggtcaggtatgaagccctcccgacaacgtagcctgaacc |
| elongatus | | gtaaggttgaagccgaatccgtgaggaggaagcaacttcaccagtgtcaggtgatagggagctaggcttgagggtat |
| | | ggtgaacgtaagtgaagtgacgctagaagcctcgtgatcttagacaggccaaagacgctgataggcctgggccaaa |
| | | atggcaaacggacaggactcactgtttgttcttcagtgaggggggacgatagctccgtcggggcagagtcaccacct |
| | | aacccctcatatgtcatctaaacggaacgtggtaagcccgtactttcgccttgataggctaagaacaaggcaagtggac |
| | | cgcggggaacacccatggaagtgcgggtaaaggaggtgggaaaaagcgaatgccaatctgtaatggattggatag |
| | | ggattgaggatggtggcaggacatccaacatcatcccactcgaaagagggcagacttcccactggtccccagttacg |
| | | agaaaactcacagaacctccctaatggtgacaaagcaaatggcggtgatcttgagtcactggtgcggtcaccaacctg |
| | | ctcaaaaggtagagaggctgtagatgagtatcgtaaaggttgcatgcacaaccggttcctcttaaatgaggggtcctgg |
| | | ggggctcgagccggatgcagggaaacttgcacgtccggttcctagggggctagggggcagcaatgcccccctgct |
| | | acccgaccgacatttct |
| Azotobacter | 157 | tgaacgcttagtgcgccagttaaagctggcggaggatagtcgatgaaagttcgatactggtaagaaatggcgaatcaa |
| vinelandii | | ccagcccccgagtcatgcacgttgcaccgtgaggtgcagggtgaagcgttgacaggggaaaccgatgggccagcc |
| | | attgagccgcgtaatcagaaatccgggacgccgatgctgttaagcgaagcagaaggcaatacggggcatggcgttat |
| | | acgccagtcatgccctggtcctgcgcggtcggagaccctgcgcacgtcgggaagtccttcgcacagaaactgggag |
| | | atctcagtgatgcccggtgcgcatgcactgggcggagcaggcaaggccaaaagctgtaaccctgctgtctacgtcgc |
| | | tgagaagtcggatacgtccgtagtacctgagaaaccgtcgaacaaaggggctagccctgcggagagggtggagga |
| | | aaggggcgtagccaaggggaacaccgacaagaaccccacgccccggacgctgagccggagcagttgcgtgtcga |
| | | tgggacttgaaggtgtacgtgaagcagcccgtagggacaagggtatgcagttcacggcattgttgcaccacatcacac |
| | | cgcaactgttggcgcagagcttctacgcacttcgccgcgacgcggcggtgggagtggacggtatgtcgtggcgaga |
| | | atacgaagaagacctccaccagaggggggcaagttgcacgcacggctccacagaggagcctatcgggcaacgcc |
| | | gtcacggcgggtatatatccccaaagctgatggcaggcagcggccactgggtattgcctctttggaggacaagatcgt |
| | | acagcaggcagtcgtcaccgttctgaatgcgatctatgaggaggatttccaggggttctcgtatgggtttcggccggga |
| | | cgaagccagcacgatgcgctggatgcgttgacggtcgcgctcaagagccagaaggtgaactggatactggacgcg |
| | | gacatcacgtcgttctttgacgagatcgaccatgaatggatgctgatgtttctggggcaccgaattgcagaccgacgca |
| | | tgctcggactcatctgcaaatggcttcaggcgggagtaatggaggatggccgtaggttggctgcgaccaaggggact |
| | | ccccaaggcgcagtgatatcgccgttgctggcgaatatctatcttcactacgtgctggatctgtgggcaaggcagtggc |
| | | gccaacggcatgcccgtggcgagatgattgttgtgcgctacgcggacgacagcgtggtgggtttcaggacgcaatg |
| | | gcaggcgcagcgtttcctggtgcagttgcaggaacgcatggccaggttcggtctatctcttaatgcctcgaaaacacg |
| | | gctgatcgagttcggtcgctttgctgtacagaatcgcaggcggcaagggctgggcaaaccggagacgtttgatttcct |
| | | gggcttcactcattgctgtagtaccaacagaagcgggggatttcaaatcctgcggctgacggtcaagaagcggatgc |
| | | gtgcgacactgcaagccatccggatagcactgaatcgccggcgacatgagccgatccgagtcgtaggtcaatggctc |
| | | ggcagtgtggttggaggatatttcaactaccacgccgtgccgggaaacctgatccgtcttgacggttttcgggtggcgg |
| | | tttgccgtctttggcggcaagccctcaaacggcgcagccagcgtaaccggctccagtggtcgcgctatggacgccttg |
| | | cagacctctatataccaagacccagaactgcacatccttaccctgaggagcgcttcgcgtcacgtacctgaggcagga |
| | | gccgtatgcggtagttccgcacgtacggatctgtgcggggggggcaggtaactgccattcctaccgcgacctatagg |
| | | aac |
| Bacillus | 158 | gcaggtcgtggtgcgaatcgttcctaagtgaaaagcttaggcatcttagacaagggaaggccccattaagatagaact |
| cereus | | aatgattcgagagacagaatgatagagtaacgtcttgaaatgtttcccctaagtctccgacatgcattgaaaggtaggat |
| | | gtttaaatgtgtgaagctcggtgaagacggctgacagataccgtagtgaaaaatgtgttttaaccgaaagtccttttagta |
| | | agaggatgtatcccataggccgggggtccgtaaagtatctatggtgagaatgttatatgaaataactgacgaactttcga |
| | | attaacgagtctataaccatacaatcagaaatgatagtaaaccatattttgtgttgtgtgtgaggttaagtaaattcgcggct |
| | | atgaacaaccttacagtattataggtactgtctagcacgcaggctcatagaaggcacctaagggtaccatatagtggat |
| | | agaatcattggaacgatgaaagctcaggacgcagagaacctacattctgtgaagcggtggtaaggaaaaggaggaa |
| | | tcctataacttatctttatttgagtgatagcgatgtcatagtagcgtggaatttatggaaacataaaggagcgaagggcatt |
| | | agtcaatattgtgaggaaaaagcaatattgatggcaagccgtatgaagggaaactttcatgtacggtttagtgtggggg |
| | | aaaaagcagagattatatcaaagttttacctatcacaataaggagagaa |
| unknown | 159 | gggtcgggccgaaagcgccgattaaaatcggtattggatagagaatgaaagagtcttatggtggaaggtttagcaaac |
| bacterium | | cacaccagcctcgagtcatgcggcaattgcagtaatgcattggctgaagcgttgacagagggaagcacaggcgggc |
| | | tattgagctccgaaatcactcattctcgggtgccgaccacgtggtccgatggggaaggcaacacgttttgccgcgttatt |
| | | atgcgagctgtaagacgacccggcggagtcctagaaccttgcatgtgtggacatcctttacgcgagaatcgggagatc |
| | | cgtgagtcttcctcaaaatgaaaggaacggccagtgaaagagaaattctgtacgcctggcatgaacgacttacggaag |
| | | tcggacaacaacatagtacctgagaagcttgcgaacaaggattttaaacatccgcggagcaggtggagggaagggc |
| | | gttgaccaaggggaacatcataaacgctgccgcagtctgtacacagggacaggtagctgcgtcgaccggcctttgtg |
| | | gtgtgtgtagaaaagctcagcaggacaagagcgccagatttaacaatctttttcaccatattacgccagagctgttgactt |
| | | caagtttctacaacctgaatagaaaagcagctgctggagtagatgaaatgacatggcgcaagtacaaggaaggctcg |
| | | cctggacgaatagcagatttaaatgaacgtgtgcataccggaagctatagagctaaacccgttagaagaagctacatta |
| | | ataaatctgacggccggaaaaggccacttggtgttactgctctggaggataagatagtccagcaggcagtctcaacga |
| | | tattaaatcaaatctatgagaccgattttatgggtttcagctatggtttcagagaaaaacgtagtcaacacaatgccctcga |
| | | tgctttatatattggaatatcgaggcgtaagataaattacatacttgatgcagatatctctggattttttgataaaatcaatcat |
| | | gactggttattgaaattcctggagcatcgagtagcggatcgcaaaatattgcggctgattaaaaaatggctaaaagtcg |
| | | gtgtgattgaagatggtaaacgaacatcacttgaagttggtaccccacaagggtctgtgatttcacccgtgttggcaaat |
| | | gtttacctgcactacgcacaagatctttgggcacaccaatggagaaaaaggcatgcagacggagacgtgattattgtg |
| | | cgttacgccgatgatagtgttgtagggtttcaatacagaaaagatgcagacaggttcttgaaggatctgattgagagaat |
| | | gggtcagttcggtctcgaactacacccagtcaaaactcgattaatagaatttggacgctttgcagtggttaatcgccgga |
| | | aaagaggcgaaagaaaacctgaaacgtttgatttcttgggcttcacacactcctgctccaaaacacgcactggacgatt |
| | | cttcatacgccgtaaaacgattaagaaacggtttgtccagaagtataaggaagtgaagcaagaactgaaggaacgtat |
| | | gcatgatgatgtgaaggaaactggaaaatggctgaattcagtcatcaggggatttcaaaattactatgcagtccctggta |
| | | atatgaatttggtaaagagtttctatgaccaaacaacgcgggcatggctacataatctaagacgcagaagccacaaag |
| | | ggcaaagtttcagttggttgcgctttacaaaattaatcaggtggttgataccgagagttcgaattgcacatccttttcctga |
| | | ccagagatttcagcgttattacctgaggtaggagcccagtgcggtagttccgcacgctgggatctgaacggggggtg |
| | | ctgggtaaccggcattcctaccgtgacgtatcaagtg |
| Methanosarcinales | 160 | aaaatgataggagtgccgagataaatgcataatatccagttggtgaaaatccaacctgaggaaaggttagccgccagc |
| archaeon | | tcagaactgaaccttgcgcccagcctggtaacaggctgtggtgaagccaggggaatgggatactgggccgcaacgc |
| | | aagtgaaggaattcagccacgaaatatttattgtcccggaggccgacgttgtcttgttgacggaaggcagtactctcatc |
| | | gccgatagtcttattgggacatggcaaggcgatgcgagcccggcggggtctaagaccgtggcacggtatcaaacgg |
| | | atattatggaaactcgggagacccagagtgttctccttacaggagtatgtcctatcaagcctataaacggtaagactgta |
| | | cagacgacactctgggagtcggaccagttcatagtaccggtgatgtggggtaatgcccatagagggaagggactgg |
| | | caggagcgcgatggaccggcagggacacatcccccatacccagagatgggcaaggggtgtcaacaaaactgtggt |
| | | ccataactcaacgagccagagaagatccaacctgtaagtttacgtctttagcgcacttacttacggcggattttctcaag |
| | | gaatgtttccgggagttaaagaaagataaagctcccggcgttgatggggtaacatggagaaagtatgaagagaactta |
| | | gacgagaatacagaagatttggtaacaagattgatagcgaagcaataccgaccacaaccagtaaaacgagcctatat |
| | | accgaagtcgaacggggaacgaagaccgctaggaattccagctctcgaagataagatcgtccagttggcgataaag |
| | | aaaattctggaggctatctttgaagaggacttctgtgacgtatcctacgggtttcgacccaaccgcagttgccatgacgc |
| | | actggacatggttgacatgatcatcatgaccaagccggttagctatgtggtggatatggacattgcgaagttctttgatac |
| | | ggtggatcacgaatgcctgatggaatgcctgaaacagcgagtagtagacccaagtctgctgcgaataatcgcccggt |
| | | gcttaaaatcgggagtgatggaggaaggaaaatatctggagacggataaaggcactccgcagggagggatattaag |
| | | tccgatacttgccaacatatacctccattatgcgttggacctttggtttgagaaagaggtgaaggagcagctaaaggggt |
| | | ttgctcagctcatccgttacgcagatgacttcattgtctgttttcaacacgatgatgaagccagagcgtttgggaaaacatt |
| | | aagggaaagactggctaaattcggactgacgatttcggaagagaagagcagaatcattaagtttggtcgctatgcctgt |
| | | cagcaagcaagaaagcagagtaagaaatgtgcaacttttgacttccttggttttacgctctactgcgataaaacccgaaa |
| | | cggtaaatttaaagtggggcgaaagacatctagcaagaagttcaggcagaagatgaaaataatgaatctgtggctaaa |
| | | gggcgttcgaaatcgtgtgaaattagagttatggtggctattgttagcacaaaagatgattggacactaccagtactatg |
| | | gtataagtggcaatatacggggattacggagtttctactatcatgcgacggaactcgcctttaagtggataaacaggcg |
| | | aagccagaggaagagctataattggagtcaattcaatcgttttctttcttttaatccactgccaaaaccgaagatatatcatt |
| | | tctatgccctatcaaaacaaaagtagaggatgtactcctgaagagccggatgagggaaaacttcaagtccggttctgtg |
| | | agggggctcatagtaaccttggagccaatactccaacagaaggtggactatgagccctactcgacatacagaccc |
| Methanosarcina | 161 | ggacggaatcgtgcgacgagaagttatgccatggattgcggtacaatccgcctttctatccattcagaatgtctctactct |
| acetivorans | | gatgtgcatctctggcaacggatttgtgcaaagctcagaggacaacgaggtgtaaaactgaacctgacagttccaaac |
| | | cgctagggaaatggacatatggagaaccgaaaggtgaagattcacaggggtcgtgacttgcaacatgcgaaagcag |
| | | gttgaattacgcatgagccaaatggcatcgaaacccggttatgacaatccgattccgtgtcataacgaatggacgaatg |
| | | ggtttccggcctataggatcctcctaagtgttcagcaactatctatggtatagaactcggtaaaccatttgtggccctataa |
| | | ggtaggaattccgtgaggaaaaccaataccacaggtggcagaggaaatggcaaaaagctaaagattccctgtaacg |
| | | gggaatatagaggagggaaaaaaaaactctttccttaatgcgaaagcatgcccacttgccattggtcacgaaagcaac |
| | | agggattgttaattatgaatgtaagaaaaccagagataacttcggttacggaccttacagacaaagaactcacccaaca |
| | | atggaaaagcattgattggaaaagagttaaagaagttgttaataaccttcagtctcgaattgcaagtgcagctaagaacg |
| | | gaaaatggataaccgtgaacaaactctcccgtcttctgacccggtccttatatgccaaactactttcagttcgtaaagtaa |
| | | ccactaacaagggaagccgaactcccggaattgatggaattatctggtcatcgtcggcagataagatgcgttccgcttt |
| | | acaactaacgaacaagggctaccgtgcaaaaccattaacacgaaagtacattcgaaagaagaacggtaaactacgac |
| | | ctcttagcataccaactatgtatgacagagcaatgcaaaccctgcactctctggtgctaggtccaatcgaatctgctatag |
| | | gtgacaagacttcgtttgggtttaaaccttaccgctcaactaaagatgcttacgcctaccttcacatctgtttaagcaagaa |
| | | aattgctcctgaatggattgtcgaaggtgatattaaagcctgctttgatgaaatcaaccacacttggatacttgacaacatc |
| | | cctatggataaacgaatccttaaggagtttctaaaagccggatatgtcgagaattatcatctgtttcctacggaaaaaggc |
| | | acaccacaaggaagccccatatctccaataattggaaatatggccttaaacggcttagaaaacgccttagcaatgagat |
| | | tttactccagatcagatggaacaattgacaaatctcatcaaaacaggcacaaagtcaattgtgcccgttttgctgatgact |
| | | ttgtggcaactgctgattccccggaaacagctcttgaaataatcgatgtcatccaagaatttttagatcctcgtggacttaa |
| | | gctcagcgaagaaaagactcttgtaactaatattagtgaaggattcaattttttaggctggaacttcaggaaatacaaagg |
| | | aaaactccttccgaagccatctaaagactcccaaagggaaattatcaagaaaatcagtgatgtaattcacaaagcaaaa |
| | | gcatgggatcaagaccgactgatacgaatcctcaacccaatcattaggggatggacacagtatcacaaccatacagttt |
| | | cctctgagattttcagcaaacttgatgatacagtctataacatgcttatctcttgggcgaaaagaagacactcaaataaag |
| | | gtctcacctggataatgaccaaatactggcataaatccggtagcagaaaatatgtattctgcacagagctaaagacgttg |
| | | gagaggttctccaattccaaagttgttaggcaaagactagcaagccttaacaaaaatccatttatcgacaaggaatatttc |
| | | gaacaatggaaattcatggaataccaccgaaagaaacgcatcaccaacccaattctgttctaaactgacacccgaaag |
| | | ggtagtagtggctcgagccggatgatgggaaactatcaagtccggttcctagaagacggggtagggagtaatcccac |
| | | cctgttattcgacgtacgcgcca |
| Methanosarcinales | 162 | tggtctctgtttgttgacaagaagttgcatcacagattgcccaactggctaccccttccattcggggtaatcctaccgcgg |
| archaeon | | aatgcatggatggcaacgcccacgcagtaagcctgcggaacaacgtggaaaatcagaaaatccgattcggatcaact |
| | | gctaggatacgaatgctatgaagagtgtaatgcgaaccatcgaaagagtcgtaacacaggataagcgaaagcggact |
| | | gttacgcttagaccaaaaggtacgaaatcagactataactgctgcagagacgttatagagggtggacaaacggtttcc |
| | | ggcttacagatggcttctaaggcgttcataattatctgtgacgtagaacttggtaagccccctaagctcccggaatatctg |
| | | ggtagagaccccgtgagggcgaacgatggcatagggggtagaggaatcgataaaaagcgaatgacatcttgtaatg |
| | | aggtgcatagaggttcaaaatttgccttaacccaaaagggtgctgacttatcgagtggtgtctcattgcatgaacggagg |
| | | caaacctgtgaatgtaagtaattcaattacgcccttaaaaggcgagaagcttacagacaagcgaccaaaaccccggtg |
| | | ggacgataccgactggaagagggtcgaagaacatgttaacaggctgcaaaccagaattgcaaaagcagttaagcaa |
| | | gacaagtggaatttggtcaaaaggttaagatttctgctaactaactcgttttatgcgaaattgttggcagtnaagcgtgtaa |
| | | cccagaatagagggaagagaacagctggaatcgatggcgtgaagtgggcaacactgaactccaaaatgaacgctg |
| | | ctcttatattatccgatgtgaagtataaagcaaagccgttaagaagagtttacatctcaaagcagggaacgacgaagaa |
| | | gaggcctttaggaatcccaaccatgtacgatagggcgatgcaagcgctgtacgcccttgcgttactaccgattgcaga |
| | | gacgacatctgatccacgttcgttcggcttcaggatacatagaagcacacaagatgtgcgccaatatgcatattgttgcc |
| | | ttggtgggaaatactctgcaaaatgggttttggaaggggatatcaagggatgtttcgataatatcgaccatgactggcttc |
| | | tgaacaacatcccgatggataaatcgattctaaagcaattccttaaggctggatttgtgtataatcgacatttgaatcctac |
| | | ccccgcaggaacacctcagggaggaattatctccccgatactggcgaacatgacactggatgggatggaaaaagct |
| | | atctcatctgtgtattacgtcggtaagaatgggaaaatcgataaacatcgatataatctccacaaggtgaatttcgtgaga |
| | | tatgcggacgatttcatcgttaccgcaaactcagaagagacggcaaaggagattgcggagttgattaaagagttcctga |
| | | aggcacgaggtctggaattgtcagaggaaaagactcatatcacccatatcgattgtggctttgactttttgggctggaac |
| | | ttccgcaaatacggagggaagcttctgataaagccatccaagaactcgatggggaatctcatccgtaaaatcggtgat |
| | | gtgatcaagcgagcgaaggcatggaaacaggaagacctcatcaacgtattgaaccccctcattactggctggtcgaat |
| | | tatcatcgatcggctgtagctaaggagatattcagcaaattagatcatattgtctgggatatgctctggaggtgggctaag |
| | | aggagacacccggacaaacgtaatacgtgggttgctaatagatattggcattctgtgggaactcggaacagggtgtttt |
| | | ctaccggaaggaataggttgaaactattctcggatacgaagattgtccggtgtgctggcttgaaattggataagaaccc |
| | | ctttattgaccaagactactttaacttacggaactgctgcccgatactgaaagggttatgagatgcttgagcggtatgagg |
| | | tgaaagtctcacgtaccgttctgagatgaggaggggcgggtaaccgccctattcttaatctgcggctatcctt |
| Methanosarcina | 163 | gctgatgactgtgcgccacgaaagcgaatccgtgtgacagaatgtcgcatagtgtaactatgctgcatagaagatgag |
| acetivorans | | ggtgggcccctcggaatcgctatacaggtagagattccaaaccaccataagctgctgtggtcaaaagccatggtagtg |
| | | agcgttatggaaaaggcgagacttgacctcgtcaggtatgtgctatggagacgaacgcaatgaaccactgataaagtg |
| | | tcgaaagcgtagggatgtcatcaaaaccagggggtagtcgttaacctgggataagtctggaagaaacctgtttactga |
| | | ccagatggtggccggcatgaaggtggcgtgaacttagcacaggcttctgtgtggaacgtgggaacctgtacttcgatg |
| | | gtaagagaaaattccaagcggaggacccgtaaggaggaaagtatcgaagcgatgtacaggggcggaacaatccgt |
| | | agtagtgatgaagctcctgtaatgggagtgaagcgaagggattgtgttatccagttttgaaaattggtcaacctgacaaa |
| | | tcaagggaggaacccatggacgaaacaaagccttatgaaatctctaaagatatagtacaagaagcttttcagagagta |
| | | aaagcaaacaaaggtgctgctggtgttgatgatgaaaacattgcagcttttgaatcagatctaacaaacaatctctataa |
| | | gatttggaatagaatgtcctctggctgctatttccccccatcagtgaaagcaatcgaaatccctaaaaagagcggcgga |
| | | actcgcattctgggaattcccacagtactcgacagggtagcacagatggttacaaaaatctatttggaaccgcaattaga |
| | | accactctttcaccccgactcttacggctatagacccggcaagtcagctgcagacgctcttgctgcaacacgtaagcgt |
| | | tgttggagatataactggttactggaattcgatatcaaaggattgtttgacaatattaatcacgatctgctaatgaagcaag |
| | | tcagtatgcatactgacaaaccatggatcattctctacattcagagatggctcaaagcaccctttcagatggcagatgga |
| | | acagtcaatgaacggacaaaaggaactccccagggaggcgttgttagtccgcttcttgcgaatctgtttcttcattatgct |
| | | tttgaccagtggatggatagtcatcatcggtataatccatttgaaagatacgccgatgatagtgtcatacactgccgaag |
| | | caagaaagatgcagatcgactgaggattaagttagaaaagcgtttgtctgaatttggacttgaactacatccaactaaaa |
| | | cacggatcgtctactgtaaagatgacgatcggcaagaggattatcctgaaacaaaatttgattttctcggatacaccttta |
| | | gaccccgtaggtccaaaaacaagtatggaaaacactttatcaacttcactcctgcagtcagtaatactgctaaaaagtct |
| | | atgcagcaagaaatccataattggcgtatgcacctcaaacccgatttaacgttagaagacctatcccatatgtataatcct |
| | | atagttagaggctgggtcaactactatggtctcttttacaaatctgaactgtactgtgtactcaagcatatgaatcgtgcctt |
| | | gactcgctgggccttgcgcaaatacaagaaactttcagggcacaagcgacgagcaagatactggcttgggaaaattg |
| | | ccagaagggatccaaaactttttgttcactggcaaatggggatttttcctgaggctggataatgggagccggatgagct |
| | | gagaggttcaagtccggtgctgagagggcctgggggtgaaactcccccgggctactcaccttattgttac |
| Bacillus | 164 | gtgcgacacgtttctttataagtgtgcaaacacgaagtaggagggttatcaatttgatgacaacaacggattgactgcaa |
| cereus | | gtaggaatgaaagccgtcaggttgagctgaaactacttatctgatactcctatatgcaaggcgtgatagtagtcacaaat |
| | | tgtatgaagctaggtgaagtcggctgaacaaaacctaagtgagaaatcatatggtaatggataggtcgggatgctaca |
| | | aaacatctatggtgagaatgtcctaacggactggcgaatgtacaggtttaaaggattaattcattagaaatgtgtatattgt |
| | | caacgagtagttatttcgacgctatctaccgaaaagtaagagtaaataatatgaaattcggaagatctaacgatgaggat |
| | | gtaaagataacaggcttacagcaagcacctaaagatatatgtatagctaagtcattcagaacgtggtaagcaagagact |
| | | gtcacaaatgcctactaacagacaaggtgcatataaggttctaacgaaccaaaattgctttattcttgtgaaggtgggga |
| | | cacagtaccgacgaagcatgtaacaaatgtggagggatagtccctagtcttgttcgttgaaaactaaatcaactggatat |
| | | aactcacaggatcgagtaagatgatgtgacttttcgtaagaaaagggaatgaatacgttggtgagtacaacatggtttgt |
| | | aatctagttgttttagtataaaaggataagatggggcgctgtatgcgatgaaagtcgcacgtacagtgtcaagcggggg |
| | | aaaagatggagataactttaaagtcttacctatcgcaacggataaaaag |
| Bacillus | 165 | cctatcactagcgcgacacgttcctaagtgaaaagcttaggcactgtcgaactcaacagttcagcagtgaactgtcattc |
| thuringiensis | | taagaagtcaaatgaaggagtaacgtctggaagggcttcccttaatcctccgacatgcaggaaagtaggcaagtactg |
| | | aactgtgtgaagctcggtgaagtcggttgaaggttaccgtaaattagtatctctaatacgaaagctatccagcggtggat |
| | | ggtgtaactgatagaccggaggtctataaaacactcaaggttaggatgcgcgatgaactagaggcgatcgctagtaag |
| | | cgcagacgaatccctgatggtacgggtctatatcgggagggaatcgaaaggttctctgacacaaataagtgtcgctact |
| | | gtgggtgagtaaaactctcctttatgaaagcccatatatcgttacaggcgttattaaggtagcaggctcataggggaaac |
| | | ctaaaagtgtatgtacagataagaatgacggaacgtggtaagctgccgacatggagggcttgttctctttgaagtgttgc |
| | | caaggaaagtcacaatgagattagttgtcgatataacttggtttaacggcagtgaaagtggtggcacagtaccgatgaa |
| | | acgtgtaatgaacgtggagggatagccactagtcgattgaagattgaaggttactattggttaacatggtttcgagtaag |
| | | actaagagatgtaatgctccaaagtaataaggaggttacagcccatgttaaagaaaaccaagctaagacataacgaat |
| | | attatgatacacaaaaaaagtgtatgacaatttatactcgaacagtcttaacggtaacaatttctttcaattggaaacgatg |
| | | gaacgccgtatgcccggaaacgggcgcgtacggtgtggagtgggggaaaagctggagataatctcaaaggcttacc |
| | | tatcactatcgcgacacgt |
| Onion | 166 | tattgcaaaagggcgagaaagatgcctaatttcttagtaaattaaccacttacaactgcaattagtaatgcagtacaacat |
| yellows | | atttttggtctattttgtgaaagcaagatagattatagcagaaaataaaagcaaaaaaacgagaaactgtttatcgtttgga |
| phytoplasma | | tttgcgagttgaaaatagtcatggaaaagctttgcaataagcctaagagaaaaaaacgggcagtagctataaggaaaa |
| | | acagttgataccttatattttagggcagtttatatattttttgcgtatataaaatttactaacacttattgatttaagtggcaagc |
| | | aaaattgctaaacctaaaaccaccacaaccacataatttttttatttttgtatattaattgtggttatctctcctaaaactattaa |
| | | ggtaagaaattagagtaacttctatcttcctaaaagttatttgctaaaagaaagttatttgataaaagtataaaagcatctaa |
| | | aataaacttaagtgtttaggctcaaggaaaaagcagaatcgtagtagtcgtaagaaattcaaagcttagctggaatgcat |
| | | aagtggactgacttaccaaggaatttgggaaaaccaaatacaaggcgaaagaagaaatatattttatataaacgctgtat |
| | | gcttggaaacttgcttgtacggtgttgtgcgaggctcaaaaaaacctaattattagctaataattaaggcgttttgtttctatc |
| | | gcgataattactcaa |
| Thermosyn- | 167 | agacattagggtgcgacgcgaaagctagccagatgattgtcccactagcccaacaagctagaacgggaccggttgtg |
| echococcus | | cccccaaccgtagcctagggaggcatgcgtgactggtaacggtcaggtatgaagccctcccgacaacgtagcccga |
| elongatus | | accgcaaggttgaagccgaatccgtgaggaggaagcaacttcaccagcgtcaggtgatagggagctaggcttgagg |
| | | gtatggtgaacgtaagtgaagtgacgccagaagcctcgttactctcagcaggccaaagacgctgacaggcctgggc |
| | | caaaatggcaagtggacggctacacctctcctaattaggtgtacggggacattagctccaccggggtaaagtcaccac |
| | | ctaacccctcgcgtcatctggttggaacgcggtaagcccgtacttccgccttgataggccaagcacaaggcaagtgga |
| | | ctgcgaggaacacccatggaagtgcgggtagaggagacgggaaaaagcgaatgctagcctgtaatgggttagatag |
| | | ggattgagaatgctggcaggacattcaacatcatcccactcgaaagagggcagacttcccgtcggttccccattacga |
| | | gaaaatctatagaacctccctaatggtgacaaagcaaatggcggtgaccttgagtcactggtgcggtcaccaaccctc |
| | | ccaccaagggtaggcaggctgtagatgagtatcgtaaaggttgtacacacaaccggttcctcttaaatgaggggttttg |
| | | gggggcctgagccggatgcgaggaaacttgcacgtccggttcttagggggctagggggcagtaatgcccccctgct |
| | | acccgactggccttgcg |
| Thermosyn- | 168 | tgacaaagcagtgcgacgcgaaagctagccagatgattgtcccactagcccaacaagctagaacgggaccggttgtt |
| echococcus | | cccccaaccgtagcctaaggaggcatgcgtgactggtaacggtcaggtatgaagccctcccgacaatgaagcccga |
| elongatus | | accggaaggttggagccgaatccgtgaggaggaagcaacttcaccagcgtcaggtgatagggagctaggcttgag |
| | | ggtatggtgaacgcaagtgaagtgacgctagaagcctcgttactccaagcaggccaaagatgctgataggcctgagc |
| | | caaaatggcaaagcggattggattcactgcttgcttttcagtgaacggggacagcaactccgccggggtatagtcacc |
| | | acctaacccctcgtgtcatctggttggaacgcggtaagcccgtatcttcgccttgaacattcaaggcaggcaaaccgta |
| | | aggaatgctgatgggggtgcgggtagaggaggtgggaaaaagcgaatgctagcctgtaatgggctagatagggatt |
| | | gagaatgctggcaggacattcaacatcatcccactcgaaagagggcagacttcccgccggtccccccttacgagaaa |
| | | gtctatagaaccttcctaatggtgacaatgcaaatggcggtgatcttgagtcactggtgcggtcaccaacctgctcaaaa |
| | | ggtagagaggctgtagatgagtatcgtaaaggttgcatgcacaaccggttcctcttaaatgaggggtcctggggggct |
| | | cgagccggatgcggggaaacttgcacgtccggttcctagggggctagggggcagcgatgcccccctgctacccga |
| | | caatggcggtg |
| Thermosyn- | 169 | gaggcgtggagtgcgacacgaagccgcttaagctgactgtcccactagcccaacaagctagaatgggaccggttatt |
| echococcus | | cccccagctgtagcctagggaggcatgcgtgactggcaacggtcaggtatgaagccccccgacaacgtagcccga |
| elongatus | | accgtaaggttggagctgaatccgtgaggaggaagcaacttcaccagcgtcaggtggtagggagctaggcttaagg |
| | | gtatggtgaacgcaagtgaagtgacgttagaagacccgttatcacatacaggccaaagacgctgacaggcctgagcc |
| | | aaaacggcaagcggacaagatacaacgcttccacgtcggtgtacggggatgttgatccgtcggggcatagtcaccac |
| | | ctaacccctcgtgtcaactaagcggaacgtggtaagcccgtatcttcgccttgaacattcaaggcaggcaaaccgcaa |
| | | ggaaagctgatgagggtgcgggtagaggaggcgggaaaaagcgaatgctagcctgtaatgggctagatagggatt |
| | | gagaatgttggcaggacattcaacatcatccgactcgaaagagggcagacttcccgctggtccccgattacgggaaa |
| | | acctacagaacctccctaatggtgacaaagcaaatggcggtgaccttgagtcactggtgcggtcaccaaccttacaca |
| | | caaggtagagaggctgtagatgagtatcgtaaaggttgtacatacaaccggttcctcctaaaagaggagtcttggggg |
| | | gctcgagccggatgcggggaaacttgcacgtccggttcctagggggctagggggcagtaatgcccccccgctaccc |
| | | gacaaaggctttc |
| Nicotiana | 170 | cggcttttaagtgcggctagtctcttttacacatatggatgaagtgagggattcgtccatactctcggtaaagtttggaaga |
| tabacum | | ccacgactgatcctgaaagggaatgaatggtaaaaatagcatgtcgtatcaacggaaagttctgagaatatttcattgttc |
| | | ctagatgggtataaaaccgtgttagaattcttggaacggaacaaaataaagttgggtcgaatgaataaatggatagggc |
| | | tgcggcttcaattaaattatagggaaagaaagaaaaagcaacgagcttttgttcttaatttgaatgattcccgatctaatta |
| | | gacgttaaaaatttattagtgcctgatgcgggaagggtttcttgtcccatgagtggattctccatttttttaatgaatcctaac |
| | | tattaccattttctattacggagatgtgtgtgtagaagaaacagtatattgataaagaaagttttttccgaagtcaaaagagc |
| | | gattaggttgaaaaaataaaggatttctaaccatcttattatcctataacactataacatagaccaattaaacgaaacgaaa |
| | | aaaaaaagagatgatagagaatccgttgagaagtttacctgtatccaaggtatctattcttactaaaatactttgttttaact |
| | | gtatcgcactatgtatcatttgataaccctcaaaatcttccgtctttggttcaaatcgaatttcaaatggaagaaatccaaag |
| | | atatttacagccagatagatcgcaacaacacaacttcctatatccacttatctttcaggagtatatttatgcacttgctcatg |
| | | atcatggtttaaatagaaataggtcgattttgttggaaaatccaggttataacaataaattaagtttcctaattgtgaaacgtt |
| | | taattactcgaatgtatcaacagaatcattttcttatttctactaatgattctaacaaaaattcatttttggggtgcaacaagag |
| | | tttgtattctcaaatgatatcagagggatttgcgtttattgtggaaattccgttttctctacgattaatatcttctttatcttctttcg |
| | | aaggcaaaaagatttttaaatcttataatttacgatcaattcattcaacatttccttttttagaggacaatttttcacatctaaatt |
| | | atgtattagatatactaataccctaccctgttcatctggaaatcttggttcaaactcttcgctattgggtaaaagatgcctctt |
| | | ctttacatttattacgattctttctccatgaattttggaatttgaatagtcttattacttcaaagaagcccggttactccttttcaa |
| | | aaaaaaatcaaagattcttcttcttcttatataattcttatgtatatgaatgcgaatccactttcgtctttctacggaaccaatct |
| | | tctcatttacgatcaacatcttttggagcccttcttgaacgaatatatttctatggaaaaatagaacgtcttgtagaagtcttt |
| | | gctaaggattttcaggttaccctatggttattcaaggatcctttcatgcattatgttaggtatcaaggaaaatccattctggct |
| | | tcaaaagggacgtttcttttgatgaataaatggaaattttaccttgtcaatttttggcaatgtcattgttctctgtgctttcacac |
| | | aggaaggatccatataaaccaattatccaatcattcccgtgactttatgggctatctttcaagtgtgcgactaaatccttca |
| | | atggtacgtagtcaaatgttagaaaattcatttctaatcaataatgcaattaagaagttcgatacccttgttccaattattcctt |
| | | tgattggatcattagctaaagcaaacttttgtaccgtattagggcatcccattagtaaaccggtttggtccgatttatcagat |
| | | tctgatattattgaccgatttgggcgtatatgcagaaatctttttcattattatagcggatcttccaaaaaaaagactttatatc |
| | | gaataaagtatatacttcgactttcttgtgctagaactttagctcggaaacacaaaagtactgtacgcacttttttgaaaaga |
| | | tcgggctcggaattattggaagaattcttaacgtcggaagaacaagttctttctttgaccttcccacgagcttcttctagttt |
| | | gtggggagtatatagaagtcggatttggtatttggatattttttgtatcaatgatctggcgaattatcaatgattcattcttaga |
| | | ttttctaaatggaaatttgtttctaaatgatgaagagataaaaaaatttcactattctgaaatgttgattgtaatagtaattaag |
| | | gggtaaatcaactgagtattcaactttttaaagtctttctaatttctataagaaaggaactgatgtatacatagggaaagcc |
| | | gtgtgcaatgaaaaatgcaagcacggcttggggaggggtctttacttgtttatttaatttaagattaacatttattttatttaac |
| | | aaggaacttatctactccatccgactagtt |
| Euglenamyxo- | 171 | cttttaggtgttgtgttttaatcattatataagaatttacaaaaaaatttgtgactttacaaatcatattaaaacaaagttttaact |
| cylindracea | | gagttttttattttatcttttaataaactaatgttttggaataaatcgtttgtggtggttagtaatttgcaaaaaaaaattttcaag |
| | | agtagttattccaatgattttaagcgttccaaaagctttcaggctttaattttaaaatcaaatgctgcaaaatacttagcaatt |
| | | cgctatgttctacaatctaatttggataaaaaaaatttaactattgatggcaaaactactttaagttatttagagcgtatctcgt |
| | | tattagagtttttaaaaattaattataataatcggtttcctcaaactatacaaaatgtgggttctttgaacaaaaatggtgatttt |
| | | aagttaacatttacttgtacgatatcagatcgtgtatggcaaacttttttaaatttttctatgcaaccggctcatgattctactttt |
| | | catccctttaattttggctttcggtttactacggaaaacttttttatcctaaaagtcttacttttaaatttaaaaaatcaatctcttc |
| | | ctttacaaaaacgtatatactatttttctttagaaaatagattggttgattttgattttctttatttagtgaaaaagttaagaattttt |
| | | agaactattaaaattggaatctttcgtttattaaaaaaaaactactctttgcagtattgtgaagataagtatgatacattttcta |
| | | cattgttatttaatgttcttttggatggcttgctagattttggtaatatttttcattatggttatcattttttgttaattctcaaaccaat |
| | | agagagcgagtttgttttttttaataatatttcgtgttttttttctagtataggtgtttctttttcagggaagtattcattactttctatt |
| | | acaggtttcgattttttatactggagctttacaaaaaaaggaaacttttatatttcagttccttccacaaaaaactactttaattt |
| | | tcttggacgagttaaatatatagttaacaactcgaattatggtgtaaaaatcaaagctgctaaattattcccgataatcaaa |
| | | aattggtatgtttatcataaatattcttttaagaaaggttgtagctctattattttttctataaaaaagaaagtattaaaagctttt |
| | | gttaaagagtctaaacaagatatttattctgctaagagattattggataaatgctttttttttgttgttattcacagtcttaaaatt |
| | | atcaagcaaaaaaaattttttcttgcaaaacacttagtttttcgcacaggttatttaaaatttcaatgtattcactgtggcgtga |
| | | atttaattttctcataatgtttctaaaattaataatgttttcccttaatttttctagtcaagattttatctatggcctaaaatataatttt |
| | | ttcttaacaaaatctttcctttaattaattttgaaaatctcaacggattaatcttttctcatgtagc |
| Methanospirillum | 172 | agttaatatagtgcgacaagaagcagtaaatcggattgcgaacccggtttaaaatcggtaacgccacccaatccattct |
| hungatei | | gggcgtccctacctgcacgtgcgtgtttagcaataaacgggtgcgaagctgcaggaacaacgtggtgattcccatagt |
| | | ctctttgggataagctgccagggaaaggtatcatggacaaacaaacgttgaaccgctgcaggatcgttaattatatcag |
| | | gaggaaagagatttgcccctgtccccactgagggagaatcacggtatcagtagcttgaattatgctggtacgaatgga |
| | | cgaagctgattccggtctatggggggtcctaaaataccacaattatccgatttattgaacttggaaaacccaatacgct |
| | | cctaaaaaaaaaaaaaaaaaatttttttttaggtaagaaccttgtgagaggaaccacatgcgtagtgggagtaggaggc |
| | | tgcaaaacgcgaatgcccggctgtaatggctgtggataggggttcaaactttgccctgacctgaaagggtgccgacgt |
| | | gcagctggtttttcacggcgaacaggatggtttcaatgaacgggataagttcaattacgcataaaagcgaggacatctc |
| | | ggacagaaaacttgcaaaacaatggaagaaatttccatttgctaaagcaagagattatgtaaagcgacttcagacacgt |
| | | atcgcaaaagcagtgaagaacggccaatatcgacttgcaagacgactccagtatctgcttacacattcgttttatgcaaa |
| | | aatgttggcagtacaacgagtaaccaaaaacagaggtaaaagaagtgcaggagtagatggggaaaaatggaccacc |
| | | cctgaacagaaaatgaaagcagcattaacgctttcggacaagggctatcgggcaaaacctctccggagaatctacatc |
| | | cctaaaccgcaatcgagtaaaatgcgacctctttcgattccaaccatgtatgaccgtgctatgcaagctttgtatgcaatg |
| | | gctcttatgccttgggctgagaccacagcagacaagacatcattcggattccgaatgaagcgaaatgcacaggatgct |
| | | gcttcatacacttttcagtgcttaagcagaaagacttcaggtcaatggatattagaaggtgatatccgcgggtgcttcgat |
| | | aatttcgcacaccaatggatgcttgataacatccctcttgaccaaagaatccttaaccaattcctgaaagccggttatattt |
| | | atgatggaatactctaccgtaacaagtcaggtacgccccaaggggcttaatttcccccttattggctaacatggctcttg |
| | | acggcatggaaagaatgttgaaagaacactttcccggaaataaggttcatctcatacggtttgcagatgattttctcgtaa |
| | | cggcagactcacaggaaacggcactccagtgcaaggaactcatcactgaatttcttcatgaacgagggcttgaactct |
| | | ctgaggaaaagaccaaaatcgttcatatcaacgaggggttcgatttcctgggctggaatttcagaaaattcaaaggcaa |
| | | attcctgatacaaccttcgaagaaagctattgccgcaatcattgataaagtaagggtaatcattaagtcggcgaaggcct |
| | | ggaaacaggaagaccttatcaaagccctaaatcccgtaattaagggatgggcaatgtatcaccggacggtttctgcaa |
| | | gtatgacctttgggaaacttgactgggttgtccgaaatatgttgtggagatgggcaaagcgccgtcataataataaagg |
| | | gaagagatggattgccagaaaatactggcacccaacacttaccagaaaacaggtctttagaacctctactcttactctcg |
| | | aaaacttctccaacaccaaaattcaataccgaaaattcataaaactggatgcaaatccgtttattgataccgagtattttga |
| | | aaacaggccaggagttttcctctcaaagcaaagatcgatacggatgttccttcactacgcccataaaagcgggtagtga |
| | | aaaacttgagcggattgcggtgaaaatcgcacggtccgttcttggaaggctgggttagggtaacctaacctggctattc |
| | | gacatacaccaaa |
| Methanosarcinales | 173 | gcggatgactgtgtgccacgaagctgcacgagagatgagggtttggcccctcgaagcctgcttgcaggagcaggctt |
| archaeon | | caaaccgccttaagctgctgtggttaaaagccatggtggtgagcgttaaggaaaaggtcccacaagatgggatcaggt |
| | | gtggatcatggaaacgaacgtaagtgaaccgctgaggaagtgtcgaaagcgtatggacggcgtcaaaaccagggg |
| | | ctggtcgttgacctgggataagtctgggggaaaacctgcttactgcccagatggcgtccggcataaaggcggcgtga |
| | | acatggtcctggctctcgtgtggaacgtgggaacctatcccctaatgttaagggagaaacacaagtgggaggacccc |
| | | acaagtgtgagagtaccgatgcaggggaataggggcggaccagcccgtattagtgatgaagtctctgtaatggagat |
| | | agagcgaaggggttgggtcatcctgcttgatttaaagatcaaccaggagatgggaggaatctttgatgaaagcaaagc |
| | | catttgagatttccaagaaagttgtattggaagcgtggaaagaggtgaaagcaaaccgtggagcagcaggtgtagata |
| | | agaagtcgattgcggattttgagaaggatcataagaataatctttacaagatctggaatcggatgtcatcggggagctac |
| | | tttcctccaccagtgaggacggtagggataccaaagaagagtgggggagagaggttgttaggcatacccactgtggc |
| | | tgacagagtggcccagacagtggcaaagaggtaccttgagcctttggtagagccatactttcacaaggactcatacgg |
| | | gtacagacctggtaaatcagcgattcaagcagttggagtgacgcgtaagcgttgctggaggtatgattgggtgcttgaa |
| | | ttcgacatcaaggggttatttgacaatattaatcatgacctcttgataagggcggtaaggaagcacacaaactgtaaatg |
| | | gatgcttttgtacatagacagatggttgaaggctccatttcagggagaggatggcacgctagtgcagagagaaaaagg |
| | | aacaccccaagggggtgtgataagccccttgcttgccaatctcgttttgcattatgtgtttgataaatggatggagcgaaa |
| | | ttattcgcaagttccattttgccgctatgctgatgacggagttgtgcactgtcggtctgaagcagaggctttaaggctaag |
| | | ggaaaatctggaggctcggtttgggaagtacaatctcgagcttcaccctgagaagacgaggatcgtctattgtaaagat |
| | | gacactcgtcgagatgagtatcccaacacgagttttgattttcttggctttacattcaggccaaggaggtcaaagaatcg |
| | | gtggggtaagtatttcatcaacttcactcctgccgtgagtaataaagcggcaaaggtgatgaggcagaaggctcgaag |
| | | atggaaaatacacctgcgaagtgacatgtccttagacgaactgtcaaggatgctcggtcccattatcaggggctggatc |
| | | agttattacgggagcttttataaatccgcgctgtacccaattttgcgacatctgaacaggatattagttagatgggcaatga |
| | | ggaaatttaagaggtttagacgccatcgtaggcgtgcggaatactggttgggggagattgcccaaaggcagccctgg |
| | | ctatttccacactggcagatgggtgtgagacctatggctggatgatgggagcccggtgagctgagaggttcacgccg |
| | | ggttctgagaggacgtaggggggaagttcccctgcgttactcacctcattgtctg |
| Methanosarcinales | 174 | ataaatgtgggagtgccgagctaaatgcataatatccagttggtgaaaatccaacctgaggaaaggttagcagccacc |
| archaeon | | tcagaactgaaccttgcatccagtctagtaacaggctgtggtgaagccagggggatgggatactgggccgcaacgca |
| | | agtgaagggattcagccacgaaatatttattgttccggaggccgacgttgtcctgttgacggaaggcaatactctcgtca |
| | | ccgctagtcttaataggacatggcgaggtgatgcgagcccggcggggtccgagaccgtggcacggtatcaaatgga |
| | | tattatgcgaactcgggaggcccagaatgttctcctttcaagagtatgtcctatcaaacctataaacggtaagatggtaca |
| | | gacaacactctgggagtcggaccagttcatagtaccggtgaagcagggtaatgccttcagagggaagggactggca |
| | | ggagcgcgttggaccggcagggacacaccttccatactcagagatgggcgagaggtgtcaacaaaactgtggtcca |
| | | taactctacgagctagagaagatccaacctgtaagtttacgtctttagcgcacttacttacggcggattttctcaaggaat |
| | | gtttccgggagttaaagaaagataaagctcctggcattgatggggtaacatggagaaagtatgaagagaacttagacg |
| | | agaatatagacaatttggtaacaagattgatagcgaagcaatacaggccacaaccagtcaaacgagcctatataccaa |
| | | agtcaaatggggaacgaagaccgctaggaattccatctctcgaagataagatcgtccagttggcgataaagagaattc |
| | | tggaggctatctttgaagaggtcttctgtgacgtatcatatgggtttcgacccaaccgcagttgccatgacgcgctggac |
| | | atggttgacatgatcatcatgaccaagccggttaactctgtggtggatatggacattacgaagttctttgacatggtagat |
| | | cacgaatgcatgatggaatgcctgaaacaacgtgtggtagacccaagtttgctgcgaataatcgcccggttcttaaagt |
| | | cgggagtgatggaggaaggaaaatatcagaagactgatagaggcactccacagggagggatattgagtccgatactt |
| | | gccaacatatacctccattatgcgttggacctttggtttgagaaagaggtgaagaagcagttaaatgggtttgctcagctc |
| | | acccgttacgcagatgacttcattgtctgttttcaatacgatgatgaagccagagcgtttgggaaggcattaagggaaag |
| | | actggctaaattcagactgacgatttcggaagagaagagcagaatcattgcgtttggtcgttatgcctgtcagcaagca |
| | | aggaagcagggtaagaaatgtgcaacttttgacttcctcggttttacgctctactgcgataaaacccgtaacggtaaattt |
| | | aaagtggggcgaaagacatctagcaagaagttcaggcagaagatgaaaataatgaatctgtggctaaagggcgttcg |
| | | aaatcgcatgaaattagagttatggtggccattgttagcacaaaagatgattggacattatcagtactacggtataagtgg |
| | | caatatacggggattacagagtttctactatcatacgacggaattcgcctttaagtggataaaccggcgaagtcagagg |
| | | aagagctataattggagtcaattcaatcgttttctctcttttaatccattgccaaaaccgaagatatatcatttctatgccctat |
| | | ccaaacaaaagtagaggatgtactcctgaagagccggatgagggaaaacttcatgtccggttctgtgagggggctcat |
| | | agtaaccttggagccattactccaacagaaggtggactatgagccctactcgacgcatctgtgt |
| Methanosarcinales | 175 | gttctctgcctgttgacaagaagttgcatcacagattgcctaaccggctgccccctccattcggggtaatcctaccgcgg |
| archaeon | | aatgcgtggatggtaacgcccacgcagtaagcctgcggaacaacgtgaaaaatcagaaagtccgattcggatcaatc |
| | | gccaggataaagaacgctatgaagagtgtaatgcgaaccatcgaaagagtcgtaacgcaggataagcgaaagcgg |
| | | actgttacgcttagaccaaaaggtacgaaatcagactataacggctgcagagacgttatagggaatggacagacggtt |
| | | tccggcttacaggtggcttctaaggcgttcataattatctgtgatgtagaacttggtaagcccaatatgctcccggaaaat |
| | | ccgggtagggatcccgtgagggcgaacgatggcatagtgggtagaggaatcgataaaaagcaaatgtcgatctgta |
| | | atggatcgaatagaggttcaaattttgccttaacccgaaagggtgctgacttatcgagtggtgtctcattgcatgaacgg |
| | | aggcaaacctgtgaatgtaagtaattcaattacgcccttaaaaggcgagagacttacagacaagcggccaaaacccct |
| | | gtggaatgataccgactggaagagggtcgaagaacatgttaacaggctgcaaaccagaattgcaaaagcagttaagc |
| | | aaggaaagtggaatttggtcaaaaggttaagacatctgctaactaactcgttttatgcgaaattgttggcagtaaagcgtg |
| | | taacccagaatagagggaagagaacagcgggaatcgatggcgtgaagtggacaacacagaactccaaaatgaacg |
| | | ctgctcttaaattatccgatacgaagtataaagcaaagccattaagaagagtctatatcccaaagcctggaacgacgaa |
| | | gaagcgacctttaggaatcccaaccatgtacgatagggcgatgcaagcgctgcacgctctcgcgttacaaccgattgc |
| | | agaaacaacagcagatccacgctcgtttagcttcagggtgcatagaagcacacaagatgcgcgtcaatacgcattctg |
| | | ttgccttggtgggaaatacactgcaaaatgggtttgggaaggggacatcaagggctgctttgataatatcaaccatgact |
| | | ggcttctggacaatattccgatggataaattgattctaaagcaatttcttaagggggatttgtgtataatcgacatttgaac |
| | | cccaccacagcgggaacaccacaaggaggaattatctccccgatactggcgaatatgacactggatgggatggaaa |
| | | aagccatcgcatctgtgtaccaagttggtaagaatggcaaaatcgataagtgtcgatataatccccataaggtgaatttc |
| | | gtgagatatgcggacgattttatcgtcaccgcagactcgaaagagacggcaaaggagattgctgagttgattaaagag |
| | | ttcctgaaggcacgaggtctggaattgtcagaggaaaagactcatatcacctatatcgattgtggctttgactttttgggct |
| | | ggaacttccgcaaatacggagaagagcttctgataaagccatccaagaagtcgattgggaatatcatctgtaaaatcgg |
| | | tgatgtgatcaagcgagcgaaggcatggaaacaggaagacctcatcaacgtattgaaccccatcattactggctggtc |
| | | gaattatcatcgatcggctgtagctaaagagatattcagcaaattagatcatattgtctggaatatgctctggatgtgggct |
| | | aagaggagacacccggacaaacgtaatacgtgggttgctaacagatattggcattctgtgggaactcggaactgggt |
| | | gttctctaccggaaggaataggttgagactattctcggatacgaagattgtccggtgtgccggcttgaaattggataaga |
| | | acccctttattgaccaagactactttaactttcggaaccgttgcccgatactgaaagggttatgagatgcttgagcggtat |
| | | gaggtgaaagtctcacgtaccgttatgagatgagaaggggcgggtaaccgccctattcttaatctgcgtgctctgcg |
| Amoebidium | 176 | taacagacgcatgaatgtcttggaatgaaaatggaaacccacgtattggattcaaacagggtgaagtaaactttccttttt |
| parasiticum | | atggcatgtgtatttagaacttatatctttaacatctagtttccctggcattcgacaatcaatagtaagaggaaagtctcata |
| | | attcacactcttttgatacaaaaaatctccaatgcttaaatgaactaagcaatattctgtacactaatataaatggtaaaatg |
| | | gtaaaatctattaaaggagaactatttgattatttaaactacagaacattagcgtacatgataatgggagatggagcgcgc |
| | | aaaaataaagggtttgtaatatgtctcgagaatttcacacttcaggaagtcgttttactattaaatatgttagcaatcaaattt |
| | | aatttggaggacctaactattcaaactgaagactctaaaccgtctccttttaaaaaagaaaaagaggaaattaagaagat |
| | | ttatagaatctactttggaatagagggaaccaaaacattattacctttcattaaaccttatttactagaccattttgattatcgc |
| | | atatcgggaatgactaaagcacagtatgaacaacgtggtgtaaagcataaatcttaggtagtaggag |
| Mesostigma | 177 | agaaaattggatgccgcgatttttttgcataagtgatccacatttttccctaaattcttctttatttctttccgtggtcactgcaa |
| viride | | tttctgcattatcagatcctttagatatttggatagaaaaatcgttcggaaatgaaaaatttttttattcacgttttatggatcaa |
| | | tgtattgtagcatgtttttcagacgctgattttttaaattcaaaatttgaaaaaatatcacaaaaatcttgttggaaattttctaa |
| | | atgtttttccactgttccttttttgggtggcacgctaattcactcttcaacaaggaaacccattgaatctaatgagctaaaaa |
| | | gtattaataatattaacagttcttggaaatggaaaatttcatggaatgttcttataaggttttgtcaaaatgaaagatattttcat |
| | | caaaattctgcaaaacctacggctgtgccctaccttgttccacgaactcatgaagagattttggctcattataaaaaattaa |
| | | tttttcttttttatctttattacaatcctatgcaagatcaccaagcattcaattgtttagtagatggtttaaaggaatcatgtatatt |
| | | gacattggcccttaaatataaaatgaaaagtagaactaaaataattcaaaaactttttcctcaaaagctgtaggagtttttct |
| Azotobacter | 178 | gcatgatgtagtgcgacgagagtcgctataggtaggagtacctaggggctacatcctaggcggtcaccacatggttg |
| vinelandii | | gtggccgcttttcccgcaaaacagctctaaaaacccgagatccgggcgacaaaagcccggattcagggtgccgtcaa |
| | | ggcgctaaggattcggcaattcgttgcctaaatatccggtcttccaccgtggagaccataaaacgatattcgtactgagc |
| | | ctgtatttcgcgttttgagtacaggggaggtatgggagatcgtgtccaaaagctggctgctcctatccgtagggcgcaa |
| | | gccatagctgtcattgtctcacccgaaaaccgcaaggtggccggggcgcgagaccttctgttcagccagaaagtagc |
| | | ctagggacagtgcgtcgccggtaacggcggggtgcgaagcgtcctgacaatgtagccccatgatgtctgtggtgcgt |
| | | ggtcgccgcgaggcgccatgcagaccctgcgagcaacacgcgggcgaggcgctaggctgggtggtacggccaa |
| | | cgtaagtgaactgctgataaacaccgtcaaccgcatggagccaaagttgctgacaggcttcgaccaaaagggtgcgt |
| | | gactggttgtccctcttacatctggggacacgctgacatcgagtcaccggtgaacaggcagggcctaacccactcttgt |
| | | gatggttatggaactcggtaagcccgtattttcgccactggctggtagagcgggggcaggtcgaccgcaaggaaggc |
| | | tgttgaaagtgcgggtaaaggaggtcggaaaaagcgaatgccgccctgtaacggggctggataggggccgttgccc |
| | | cgacccgaaagggtgcccacttccgactggtctttgatcgcgaaaaggcttgtaaagccataaaagctggcgtcgttc |
| | | ggtccgaccggcggcgtctgaacagaaatcctcgcggggtagtgaactcactccccgcaagggcgacgtgcgtcc |
| | | gctgctcctccggggaaatcttctttggaggaaagcaagatgagtgcgtctgaaaaggaagtacctgcgtcctccctcc |
| | | cggccagcagttggcacacgatcgactgggcaaccagtcatcggcgggtgagagggctacaggtacggatcgcga |
| | | aggcagccaagaatcggcaatggcggcaggtgaaaaccttgcagcgcatgctggtccgctcgttcgccgccaaggc |
| | | attggcggtcaaacgggtcactgaaaaccggggtcgcaggactccgggcgtcgacggggaaacctggagcacgc |
| | | ctgaaagcaaatggaaggctatttttcggttacagcgcacgggctaccggcccaggccgttgcggcgggtgtacatc |
| | | cccaaagccaacggccagcgccgaccgctgggaataccgacgatgctggatcgggccatgcaggcattatacctgc |
| | | tggcgctcgaaccggtgtccgagaccacggcggaccggaattcctatggctttcgtcctcaccgttccacggcggatg |
| | | caatcgagcaattgttcgtcaacctcggtcgcaagcactccgcgcagtgggtcatggaaggggatatcaaaggctgtt |
| | | tcgacaacatcagtcacgattggctgatcgccaacgtcccgctggacaaggcggtcttacggaaatggctgaaggcg |
| | | ggatacctggaatccggccaattgaaccccaccggggcgggaactccgcaaggaggcatcatctcgccggtgctgg |
| | | ccaacctggcgcttgatggtctggaaaaggcgctcgaatcgcgattcgggcaacgcaacaccaaagccagctacaa |
| | | gaccaaggtcaactatgtgcggtatgcggacgatttcgtcatcaccggcatctcgaaagagctgctggtgaatgaggt |
| | | gaagcccgtcgtcgccgccttcatggcggagcgcgggttgagcctcgcggcggaaaagtcgctgttcacgcatgtat |
| | | cggaggggttcgatttcctcggccagaatgttcgcaagtacggtgacaagctgctgatcaagccagctcaccggaac |
| | | gtcaaggcatttctggccaaggtcaaggcgttgatcgaggggaacaagacggctccggccagtctgttgatagacaa |
| | | gctcaacccggtcatccggggctgggccaactatcatcggccgatagtcgccaagcagaccttcaattacgtggatta |
| | | ccggatatggaagttgttgtggcggtggtgcaggcggcgacatggaaatcgctgcaaacgttggatcaaggagaaat |
| | | acttcaagcgcatcggtacgcgcagttgggtgttctccgggcggtatcccagcggcaaactggccacattgctgtacg |
| | | ccgacgacacaaccatacagcggcacaagaaaatcagagcggaggccaatccgtacgacccggaagacgagatg |
| | | tacttcgaggaaaggctggaatacgcctggcggagatcggacgaagggaaacggaaaacgctgaggctgtggcta |
| | | ggccagagcaagcgctgcccgatgtgcaagcagttgatcacgttcgagacgggatggaacatccaccacatcatca |
| | | agcggcacatgggcggaggcgacgagctggacaatctggtgctgttgcatcccaactgtcatcgacagctacacagt |
| | | gcggcaccggctctttcgattgagaaagggcttacaaaggcttgagccgtatgcggggaaactcgcacgtacggttct |
| | | taggggaggggtggccagcaatggtcgctccttacccgacagccctgtcg |
| Azotobacter | 179 | ctttttgctggtgcgcccagcatgggcgcactcctgcgggtgcaagtcccgccatgagctggtcacagcgaatgaag |
| vinelandii | | cgaagcgcaactgcgtgagggcgaccgagcgtggggaggaagcgcggagcgtaaatcgtgagccgatgaacaa |
| | | gaaccgcatagaaggcgctgccgagcagggcaagcgggccagtaaccgcgaagctcttgtgaccaaggcgaggc |
| | | ggcgtagatgcggcggttgtgcggtgaaggagtgcgttcttacctggggagatctcgcctcatgcctgaaagggcga |
| | | cggtgtcgagccggagcgagaagtcagcagaggtcgtagtagccgcagtagcggggccgcagaggccagatgcg |
| | | aaccggtgaaggaccgaacgagaggaagtgttggaaacgatgtcgatgtcggaggccatgcaccagaagcccgcg |
| | | tgcgcggggcggcatgccgcaggccagggtgaagccccggccaaggtatcccgtggtgaagccgaaggcccgc |
| | | gacatgagatggaaggcacagggtcggcgctgctggaagcggcgctgacgcgagagaacctgcggcaagcgttc |
| | | aagcgggtgcgagccaaccggggatcggcgggcgtggacggtctggacatcgaccagacggcgcgcaagctggt |
| | | gaccgagtggcctgcgatccgggagcaattgctgcgggggacgtaccggcccagtccggtacggcgggtgatgatt |
| | | ccgaagccggatgggagccaacgagaattgggtattccgacggtgacggatagactgatccagcaggcgttgttgc |
| | | aagtgctgcaactgctgcttgatccgagcttcagtgagcacagctacgggttcaggcccggaagacgggcgcatgac |
| | | gcggtgttggccgcgcaatcgttcgtgcagtcgggccggcggatagtggtggacgtggacctggagaaattcttcga |
| | | ccgggtcaaccacgacattctgatcgaccgcctacgcaaacgcatcgacgacgcgggagtcatccggctgattcgtg |
| | | cgtacctgagcgcggggatcatggatggcggggggtcatcgagcgggaccaggggacgccgcaaggcgggcc |
| | | gctgtcgccgctgttggccaacgtcctgctcgacgaggtggaccgggcgctggagcggcggggccattgcttcgtg |
| | | cgctacgccgatgactgcaacgtgtacgtacgcagtcggcggggggcgagcgggtgatgaatctgctgcgcaag |
| | | ctgtacggccggctcaagctgagggtcaacgaagccaagagcgcggtggccagtgcgttcggccgcaagttcctgg |
| | | ggtatgccttctgggcagcgccgaagggacaggtcaagcgcaaagtggcggccaagccgttggcgacgttcaagc |
| | | agcggatcaggcaactgacgcggcgcagcggtgggcgcagcatggcgcaagtcgtgcaggagctgcgtccgtat |
| | | gtgctgggctggaaggcttacttcggactgtcgcagacaccgagagtctggcgttcgctgggcgaatggctgcggca |
| | | tcggttgcgtgccgtccagctcaaacagtggaaacgcggcaagaccctgtttcgggaactgcgcgccctgggggcc |
| | | agccacgaggtggcgcaacggatcgcggccaacagccgccgatggtggcgcaacagcggcaagctcctgaacag |
| | | cgtgctcaacctggcgtggttcgaccggcttggcctaccccgactcgcctgacctcaagctctcgaaccgcccggtgc |
| | | ggacccgcatgccgggtggtgtggcaggggcgctgccttgacggcagccccctatgccgatcatgccggaa |
| Agrobacterium | 180 | ttggacgagggtgtgcggcgaggcacccgcagcagtgtgggagccgagctgcacgaaagatgagggtaggtccct |
| radiobacter | | cgaagccgaccgtcaggggtaggtttcaaaccactgcaagctgcgacggtaaagagccgtggtggtgagcgttgca |
| | | gaaaaggcggatgttagtccgtcaggtaaggttcagagagacgagcgcaagcgaaccgctgatgacgtgtcgaaaa |
| | | gtgcaagatgatgtcgaaaccgggaggaattcgttagcccgggacgagtctggggggcgacctgatttctgcccaga |
| | | cggcatccggcatgaaggcggcgtgactcttcaccaggctttcgtgtggaacgcgggaacctgtcgctccgatgttaa |
| | | gggagaaacccaagcaggcggtccctgcgagggtgagattaccgaagcggggcacaggggggagcagcccgt |
| | | agtagtgctgaaggtcctgtaatggggctggagcgaaggggttgcgttgttcagctctggagcgtggacaaccggcg |
| | | acgggaggatcctcgtggatagagcaaagccgtatacgatcccgaaaagggaggtgtgggaagcatataagagggt |
| | | tagagccaaccaaggagcggctggtatagatggccagacgattgcggacttcgaggctgatcttaggaacaacctgt |
| | | acaagctctggaatcggctggcgtcgggcagctactttccgccgccggtgcggcgggtcgacatacccaagagcga |
| | | tgggaagacgcgcccgctgggcatacctacggtcgccgatcgcgttgcgcagatggtggtcaaacgccatctggaa |
| | | ccggtggtggagcctgaattccatccggattcctatgggtatcggcccggcaaatcggcacttgatgcgatcagcgtg |
| | | gcacggcagcgatgctggcgttacaactgggttcttgatctcgatatcaaggctttctttgatagcattgaaccggacct |
| | | gctgatgcgggctgtgcgcaagcacaccgattgcccgtgggtgcttttatacattgaacgatggctgaaagcgccagt |
| | | gcagatgccggacggaaatctcgtcgccagagaacgaggaacgccacagggggggtgatcagccccctcttggc |
| | | aagcctgtttcttcactacgcgtttgacatgtggatgtgtcggaatttcccggacattccgtttgaacggtatgctgacgac |
| | | gcgatctgtcattgtcggagcgaggatcaggcgatggcgcttcagaacgctctggacgcgcgttttactgactgtggg |
| | | ctgacgcttcatcctgacaagactaaaatcgtctactgcagggatgaaagtcggcgaggtactcatccggtctacaagt |
| | | tcgactttctcggctacactttccggccgagacttgtgagtaagaaggccgggggcatgggtgtttcattcggtcctgcg |
| | | gccagtccgacggcgctcaaggcgatacggggcacgatccgtagttggtccttgcaccttcgcagtgacaaagctct |
| | | cgatgatctggcacggatgttcaactcgtacatccgcggctggatcaattactatggtcggttctgtccttcagctctcca |
| | | acctacgctttggagtgtcgagcgatacttggctcgatgggcatcgggaaagtacaagtccttgcgtaggcataaacg |
| | | gcgatcccggcattggcttttgcgcatcgcgcagcgccaacctcggctgttcgcccattggcctctgcttcatggatac |
| | | ggtcgaacaatgggagccggatgatgcgagagtatcacgtccggttctaagagagcgtgggggtggaactcccccg |
| | | cgccactcgcctcctgatttc |
| Alistipes | 181 | aagtatttgtgtgcgcccgtacaagctgcttagtaaatatcccactggcaagggattaggtaagtgttctttgaaaaacct |
| shahii | | atcatcacccgacttatccgaaagggaaaccggacggggagtgtagcatgtcgggaacgtcgtaagtcagtcagtta |
| | | ccagactgccactgaacggcaagatgaaacgatggatatgaggatgaaaaccgaattgtttgaacgacagtccgagc |
| | | tgctctacttgaaggctatgacgtaaggtaactgaaatcgtcatactgcggtactgctccggtgtataggtacggacgca |
| | | agcaagaacttgccgcagagcaaccatgataaatggtaaaggacggaaatcgcatccgacaatccatcatgccaaca |
| | | gttactaagtaagtaaacggggattacctaaaccggaacgccgtaaggctatgaggtgcagacctctgaatatccggc |
| | | acggtaacggagcctccgtagtagtccgggcagggtaacgccctgcacatggcgaagggaggcagctaattttcttt |
| | | aatacaatttaatggataatgtgagagacattatgagaagtccggaaagagtattaaacagtctgaacgaacacagtaa |
| | | ggattcgagctacaagttcgaacgcctttaccggattctgttcaacgaagagttgttctatgttgcctaccaaaagatcgc |
| | | atcgaacggaggcagtacgaccaaaggctccgacggtcgcagtatcgacgagatgagccttgcccggatcgaaac |
| | | gctgatagcctcgttgaaagacgaaagttatcagcctcacccgtcgcggagggtgcatatcccgaagaaaaacggga |
| | | aaacgcgccctctgggaataccggcttttgaagacaagctggtgcaggaggtagtccgcatgattctggaagccattt |
| | | acgagggacattttgagaccacctcgcacggtttccgacctaaacggagctgtcacactgcattacttcatattcaaaag |
| | | acgttcagcggggcgaaatggtttatcgagggcgacatcaaagggttcttcgacaacattgaccatgatgtacttgtcg |
| | | gaatcctgcgggagcgcatttcagatgaccgttttatccgtctgatacggaaattcctgaaagcgggatacgtcgagga |
| | | ttggacgtttcacaacacttacagcggaatgccgcagggaggtatcgtcagcccgattctggcgaatatatacctcgat |
| | | aaattagacaagtacgtgaaagagtatatccgacatttcgacatgggaaccaaacgcaggccgggcaaagagagca |
| | | acgatttggccaatgaacgaaaacggactgtgcggaaactgaaaaagataaaagacgggactgagaaggcggcttt |
| | | ggtcgcaagactcaaagccatcgaacaggaacgtgcagcatttccaagcggagatgaaatggacggaagttaccgc |
| | | aggctcaaatacatccgttacgccgacgattttattctgggtgtaatcggtagcaaagaggatgcactgcggataaagg |
| | | aggatattaaatcattcctatccgaaagcctcgccctcgaactgtccgaagaaaagacgctgataacccatacgggtaa |
| | | atcggcgaaatttctcggatatgagattacggtaacacggaacaatcatcaacgacgggatgtgcaaggacgtctgcg |
| | | acgcacctacggcaagcgtgtccggctgaatgtcagcatggcaacgctgcgggacaaacttctggaatacggagcta |
| | | tggaaatcaagctccgcaacgggaaagagatttggaaacccaaatgccgttcaggattgatattcaacgacgatcttga |
| | | aatcctcgaccgatacaatcgggaaacagtgggattttgcaactattacctgatcgccaacaactgcgtcgtactgcac |
| | | aacttcagatatatcatggagtacagcatgtataaaacatttgcgggcaaatataggagcacggtacgaaaaatcaaca |
| | | aaaagtaccgtctcaacaaactgttcaccgtaaagtacgagcagaaaggggtaatcaagtcccgaaccttttacaaga |
| | | caagtttcaaacgccggacaacggcgttcaacggaagctgcgacatcgaaccgtactctatcgcagacgcgagccg |
| | | aaccaatttgacagacaggctcaaagcggaaaaatgcgaattgtgtggggcaacgggcaagctgattatgcaccatg |
| | | tccgcaacctcaaagacctgaaagagaaagagagttggaaacggctcatgtcagcccgaaaacgcaagaccattgc |
| | | gttgtgtccgagttgccacaggctgcggcatctgggaaaagtttagactgaaaaaaaaaattagtggagagccggata |
| | | cgccgagaggtgtaagtccggttcgggggcgagttcttggaaacctgccgtagaaatacggtaaggcgccgggtgc |
| | | ttagcctacataaaacctg |
| Wolbachia | 182 | tcagtaccaagtgcgtccagagaaggcgagtattgttcagtaggaaatagacctaccaggataaaagtcggaatcctg |
| sp | | tagcttgggtagcagcataaagggaaaccaaaatgttgaagcctaccgacaacattatcgtaggataatggcgaattac |
| | | caggccgtagcgaaagcgaatgcacagattgcctcgaaaggaatattctgaaggtcgagccatcttggtactggcga |
| | | agacagaatagaccagacgaaaaacgaatgaaacgattggtctacttcagcggggtgttagtaacagcatggcaatg |
| | | aggacatacaaagcaactggagaagccctcctcatcccaaggagaaacaccttggagaggtaggttctataactgga |
| | | aaacaggaagtgaactgaagatgagagggtgacggatgggtgcgtagtagtgaagaagtgaggtaacgctcatgga |
| | | gcaaaggcaccctactagcagggatcttttaacaaaaggaaggtaaaggtaaaaatgacaaaaatgccgataaaatta |
| | | caagacctgagaagaaagatatacaccaaagcgaaggcagaaccggaatggcgattctggggaatccatgttcatgt |
| | | ctgtaaaatggagacactggaggaagcttacaaactaacgaagaggagtaatggggcaccagggattgatggggtg |
| | | acattcgaatcgatagagacagagggctcaagaaagtatcttcaacgaataagacacgaattaataaccaagacctat |
| | | agcccaaataggaaccggagaaaggaaataccaaaatctggagaaaaattcagaacgttgaacataccctgcatccg |
| | | ggatagaatagtgcaaacagcactgaagctaatacttgaaccgatattcgaatcagacttccaaaaaggatcatacgg |
| | | gtatagacctaagcgtaatgcgcatgaagcagtacaaaaagtaacggaagcagcaatcaaaggcaatacaaaagtaa |
| | | ttgacgtagatctaaaatcctactttgacagtgtgcgacatcatattctgatggaaaagattgccaaaagaataaatgaca |
| | | aagaaatcatgcgcatgattaagctaatcctcaaaataggaggaaaacgaggaatggcacaaggatcaccactttcac |
| | | cactactaagcaatatatacctaaatgaggtagataaaatgttggagaaagcaaaagaggtgaccaaagaaggaaagt |
| | | accaacgtatggaatatgccagatgggcagatgatttggtgatactgatcagagaatacccgaaacgggaatggctag |
| | | aaagagcagtatacagaaggttggaagaagaattagcaaaactagaagtgagagtaaatgaagaaaagacaaaagt |
| | | cattaacctaaagaaaggagaaacatttagcttcctagggtttgattttcaagagaatatcacaaaacaaggaaagtgga |
| | | atgtcaggaaaacaccgcagatgaaagcacgaacaaacctgctacgcaagttgaaggaaatatttcggcgccataaa |
| | | tcgcaaccgataaaaagagtgataaatgacatcaatccgatattgagaggggggtaaactactttaggataggaaact |
| | | ctagtcgaaagttcagttacgtaaaacactgggtggaaaagaaggtaagacgcaatttaatgcgagccaggaaagca |
| | | aagaaagggtttggttggaagaaatggagtagcgaatggatatacaaaacgctgggtctatattcagactacagaataa |
| | | ggtattacagaccgaaagcatcaccaacacaatatgtgataaactttggtaaagaacttgctaggaaagcgtagtgcgg |
| | | gaaaaccgcatgctgcgtttgacgaggcgggagctggaaacgttaacatgggaatttggattgaggcccaggatgaa |
| | | aataatggatcagccaccaaaccctaaagaggcgcgccagttctcgaccctacagatgcgctt |
| Xylella | 183 | tggcaagctcgtgcgacgtgaagtcgcttgtttcattgcttcaccgcttcggcgtgatgaagcctgatgttccgtcatcag |
| fastidiosa | | tagcctatcggtgcatgcgtggttggcaacgactgcgtgtgaagccaccgagacaatgtaggtcggcgaaagccag |
| | | gtggagaccgcgaggtcgaaaccgcatctgcgagcgccaacgcggatggatcgctagactggtcggtatggttaac |
| | | gtatgtgaagtgctgataaacaccgtcaagatgaaagagccaaagacgctgacaggctctacccaaaatggggcgta |
| | | gctggacattcctctgagctttgggaatgcacggacacgaaagctaccggtggagaggcgcaacctaacctgacctg |
| | | tgaatcaagtggaacacggtaagcccgtatctctgcccctcggggcagacagaccgtaaggaaagttcatggagatg |
| | | cgggtagaggaatgtggaaaaagcgaatgccgccctgtaatggggcggataggggctgagccgcgaggcaacatc |
| | | gcttcacgtgaaaacgggcagacttccacttggtctttctttacgagattgtttgaagaacctttttaggagagaaagcaa |
| | | atgactacgcaagctgcttgcgcgggtgcgctctccggcgactcgggcggatggcactgcatcgactgggccaact |
| | | gccaccgggaagtcaggaggctgcaaacgcgcatcgtaaaggcgacacaggaaggccgctggggcaaggtgaa |
| | | agccttgcaatggctgctgacccactcgttcagcggcaaggctcttgccgtgcggcgggtgactgaaaaccagggca |
| | | agaagactccgggtgtggacaaggtggtatggggcaccccggaaaagaaggaaaacgcagtagatgagttgcggc |
| | | gccatggccataaagccagaccgctgaggcgagtctatatcccgaagtccaatgggaagctacggccattgggtatc |
| | | ccaaccatgagagatcgcgcgatgcaggcattgcatctgctaagtctgctcccggtgtcggaaacccaagctgatgg |
| | | atgttcctacggcttccggcccgagcgttcagtggcagatgcagtcgataggtgcttcagggcgcttaatggacaacat |
| | | tccgcccaatggatactggaggccgatatcaagggatgcttcgatcacatcagccacgactggcttctgggcaacgtg |
| | | cccatggacaagcggatgctggaaatgtggttgaagtgcggcttcatggaagcaggtgcatggtcggaaactgaggc |
| | | agggacaccacaaggcgggatcatctcgccgacactggcaaacatggcgctggacgggctggaacaactattgga |
| | | acagtccttccgctcaagaatgcgagatcggcagatcaccaacccgaaggtcaaccttgtccgttatgccgatgacttt |
| | | gtgatcaccggagactcagagaagcttttgatcaaggaggtcaagccactggtggagcgattcctggcggaccgtgg |
| | | actcaccttgtctgcggagaaaaccagggtgacgcacatcgacgaaggcttcgacttcctcggccagaatgtgcgca |
| | | agtacaaagggaagttgctgatcaagccatcagcagcgagcctcaagaattgcgcagacaaaatccgggccatcgta |
| | | cggtcgaacaaggcagccaatcaggcatcgctaattcacctgctcaaccctgtgatccaggggtgggcaaacttccat |
| | | cggcatattgttgccaagaagaccttcaagtctttggatgatcatgtctggagggccctgtggaaatgggctctacggc |
| | | gtcatccggcaaaaggcaaaaagtggattcggcatcggtatttccatgcgtagcgtggtcgtcaatgggtatttgcagc |
| | | ccgatacgtggactcttcgagcaaagaggctcgatgggcaaccctccgtaaggcagtagacaccaagatcaggcga |
| | | aatacgatgatccgcatgggtgcgaatccattcgacccggagtgggaaacctactttgaacagcgcctcggcgccaa |
| | | gatgaaggacaacctacaaggtcgaaaacgtctattgcacttgtggttggaacaggacgggaagtgtcctgtatgcga |
| | | cgaaccgctgacgaaagaaagcggctggcatgtccaccatatcgtccgtcgcgtcgatggtggttccaatcaaagcg |
| | | ggaacctggtgatggttcacctgaactgtcattaccagatccacaaccttggtttggatgttgtgaaaccggctccagca |
| | | atggggctttgaaaggcttgagccgtatgaggggaaaccttcacgtacggttcttaggggaggatgtggtggcgacg |
| | | ccatgtccttacccgaccacataggca |
| Zunongwangia | 184 | ggatgaaagtgtgtgccttgaagcggcctaaagtaatgccgcaggctgttagattgatggtggtagacccaccgtagg |
| profunda | | ggttgacagacagaccctacaaaccaccttgtggtgctttatttcgaaagaatgaggtgctgagcgacaaggaaaagg |
| | | cgagagaatactcgtcaggtgaatttcagtgagctgaacgaaagtgaatcactggttaagttaaagtaccgatatcgcc |
| | | agattctgtcgaaatcggagaattgtgtttcttctccgagaataaaacttagtggcagtaacctgaagttgactaagtgac |
| | | aggcggtacagaggtggcaggacactgaaataggcgatctaacggaacaagggaatttgtacagggatggtaagg |
| | | gaaatgcacaagcggaaaccccgtgaggcagaaaaccaatgaactgtacagaggcggactaagccgtagtactgc |
| | | agaagtctctgtaatggagatggagggaagggcttagccagattagttttatgtaagacacaactttgaaagaaggatg |
| | | attgattattatgaaacaaaacaacatccaataaccaagaaaatggtattggatgcgtacaagaaagtaaagtccaaaa |
| | | agggcagtgccggagtcgatggacagagtttacagaattttagggagaacctctctggaaatttgtataaaatctggaa |
| | | ccgtatgacctcaggcagttattttccgcctgttgtaaaggaagttcgtataaccaagaaaacaggaggctttcgcagtc |
| | | tcggaattcctacggtatcggatcgcattgcgcaacaggtgattaaaagttacttagaacccaaggtggaaagcagtttt |
| | | catcagaatagttatggatacagacccaggaaaagtgcgcatcaggcactggagaaaacagtgtccagatgtggttat |
| | | tacagttgggtggtagaccttgatattcgtggattctttgataacattgaccataccctactgatgaaggcagtagaaagg |
| | | tatacaaaagagaaatgggtgttgatgtatattggaaggtggttgaaaacaggagtatccagagaaggtgaaatcacc |
| | | gatagaataaaaggtactcctcaaggcggcgtcattagtccgctacttgccaatatatttcttcactttgcattcgataaat |
| | | ggatgcaaatccatcactccaatatgcctttcgaacgatattgcgacgatgccatcatacattgcacatcagagaaacaa |
| | | gcatatttcattagagaggctgtttctaaacggatgaaagcttgtaagttggaactcaatagtgagaaaactcacattgtgt |
| | | attgcaagaatcacgtgcacagcgaaagtcacaagaatacaagttttgattttctcggttacacgttccgccccctcaga |
| | | cgaccaaccaaaaatggttggaaactgacttactttccagtaatgagccacgcctcaaagaaagaagcaaggcgaag |
| | | acttaagaaggttgtcaatagaaagtttcggggatccatccaagaactggcacagataatcaaccctatgataaggggc |
| | | tggtatcaatatttttgcaagtattcgaaatggacaacccgtggactatggtattggttaaacaggaaaatagttagatgg |
| | | attaggaggtacagaaagcgcagtagagtgcaagctcgaaaatggctaaaaaatgtgtacaagactaacccacaatt |
| | | gtttgagcattggaaacctgtcttagatataaaaccttactaattgtaccgttaatctggaagcgccgtatgatgggagact |
| | | atcacgtacggtgctgtgagagacttggggtgaaattccccttgtctactcgacccgataaagg |
| Zunongwangia | 185 | acttgcgatagtgtgccgagcaaacaatgaccggcaataaatagtcattgtgaactataattaagatggtagaatcgac |
| profunda | | ctaccggtgtcgtcttataggagaaggcatcaaaccaaccaaagatgctttggtaaagagccaaggtattgagcgttta |
| | | ggaaacggccagtcaagaattggtcaggtatgattaagagagatgaacaaaagtgaaccccttctgaggtgtcgaga |
| | | agttgctacatcctgtcaaaagccacgaagtatcgcacggggttaaagagtatagcagttacctatttattggctatacg |
| | | gcaggcgtcattcagggggcatgactcttattcaggcttagttatggaactcgggaacctgactatagatgctaaggga |
| | | aatacacaataggctcaacctagaggtagaataccaatgctgttttcagggacggactaacccgtagtagcaatgaagt |
| | | tcctgtaatgggaatagagcaaaggggttaggttatacagttacgttacatttaacaactcagttttgaggatgactttatg |
| | | caatgaaacaaaatcaatacctataagcaaagctatgatatggcaagcttataaaagagtgcgcgccaacaaaggaag |
| | | tgcagggatcgatacagtaagcattgagcaattcgatgagagtttatcaaagaacctgtacaaactttggaatcgtatgg |
| | | catcgggaagttatttcccccctgcggtcaaagaagtggaaatcccgaaaaaggatggtaaagtccgtaaattaggga |
| | | ttcctacgataagtgaccgtatcgggcaaatggtggtaaaaatgtacctggagccccgtttagaaaatgtattcaatcca |
| | | aactcctacggatatagaccaaacaaaagtgcccatcaggcacttgaacaggtcaggaaaaactgttggaaaatggat |
| | | tgggttattgatctggacattaaaggtttttttgataacattgaccatcataaaatgatgcttgctatagaaaaacacgttcct |
| | | gaaagatgggttaggttatatatagcacgttggctcgcaagtccagtaatgacaaagtcgggaaatttagtctctaacca |
| | | gggaagaggaactccccagggaggggttataagcccattactggccaaccttttccttcactatggtttagataaatgg |
| | | ctggaacagaacgacaatacggtgaaattcacacggtatgccgatgatgtgattgtgaactgtaaaagtcaaaagcac |
| | | gcagagcaaacacttgaagctatcaaaagcaggatgcatcaaattggtctggagttacatccagagaaaaccaaaatt |
| | | gtatactgtagagattaccgaaggcaagaaaagtattccaatgtgaagtttgattttctaggttattcttatcaacctagaac |
| | | tacaaaatcgaagaaatcaaacggactctatcttggctttgattgcggcataagtataagttcgaggaaacgaattgcag |
| | | ataaacttgaagaattgaaagtagagcgaatgacttctgatcgtatagtaggaattgctgccattctcaatccaatgatta |
| | | ggggatgggtgaactactatggtaaatttagaaggtcaatgctacacaaagtcttcaagttattgaataatcgaatcgtga |
| | | aatgggcaagaaaaaggtataaacgttacaaaaccagtataaaacgtgcctatcaatggtttgaaaggattaaggaac |
| | | aatatccaaaactattttatcattggcaggtgggatttgtatggtaatttgtaatttagattgtataacaagagccgtatgatg |
| | | ggagactatcacgtacggttctgtgagaggcttggggtgaaattccccttgtctactcgacaccgtaaccg |
| Vibrio | 186 | tcaagtaaaagtgtgccgtgggcacaggatcctgttatctaaggaaggtaaacaggagaatgagcataagctcatgaa |
| vulnificus | | gagatggaggtaggccctccagagtctgctgtcggaagtcggctctaaaccactttcaagtggctgttattaagagtaat |
| | | ggtcagaagcgttgaaggaaagtcagaatagaattctggcaaggtagagtgcaaggagatgagtaatcccgaaccc |
| | | atgtggacgcgtcgttaattagaaccagcatcaaaactgaggtcgttttattatctcaggacgagtacaccggtaaattcc |
| | | gaatgctgggtgtgcggtgctcggcgtaaagtgggcatgactttgcactaggctctttgtgggaaccacgggaaccag |
| | | tcatcacgatgtaaaggaagaaatataaatgactaaattcatgagtatgagagtaccgatgcgtgatactggggcggag |
| | | caattcgtagtagtgatgaagggcttgtaatgagcgtggagcgaagggattgcgtcaagtcggttggaacgatgactc |
| | | aactgccgacaggcaggaggaggtcgcacggacaaccaaaaccttttgcaatatccaaatgggatgtaatgactgcg |
| | | ttcgagaaggtaaaagccaacaaaggcggagccggagtggatggagtaacgatagaggactttgaaaaagacctta |
| | | aaaacaacctttacaagatatggaacagaatgtcatcggggtcctactttcccacaccggtcgcagcggtaagtatacc |
| | | gaaaaagtctggtggagagagggtattgggtatcccaacggtcagcgaccgagtggcgcaaactgtggtaagagac |
| | | aagcttgaaatcatgctcgagcatcacttcctagatgactcttacggctatcgagtgggtaaatctgctcatgatgcgata |
| | | gaagtcactagaagacgatgttggcagtatgattgggtactagagtttgatatcaaaggtctctttgacaatattcgtcatg |
| | | acttgttgatgaaagcggtaaagaaacatgttcagctcgctgaagagagtcagagccgggattatcaatggataacact |
| | | gtacatcgaaaggtggttagttgctccgttacagaaagcagatgggacgcaaacagaaagagagttaggaacgccac |
| | | aaggtggcgtggtaagcccagtgcttgccaacctatttcttcactatgtttttgataaatggctggagaagaattatccag |
| | | acaacccatggtgtcgatacgcagatgatggacttgtccatgcaaggacaaagccgaaagcggagaagttgaggga |
| | | tgagctagcgaagcgcttcaaggagtgcggactggagatgcacccaattaagacgaagattgtttactgcaaagatga |
| | | tattagacgagggtcaggtaagcatatagagcataaacaatttgattttctagggtataccttcagggccagaacaaata |
| | | agtgtaaacgtacaggtcaactctataatcgattcctgcctgcggtcagtatggcagcaaagaaagtcatgcgaaggc |
| | | aaatcagagagctaagagttcgccaggaaacgcagtacagcttagagcaattaagcagatggttaagtccaatgctga |
| | | atggttggataaactactacggaaagttcaggcgaagcgaattagactcggtattcagacacttcaacaagactttagta |
| | | cgttgggcgagaaggaaattcaaatcgctaaaatgtcacaaaagtcgaaccgtagcgttctttgataagctatcggctc |
| | | aatgccctagattgtttcctcactggaaatttggttcagcaagaagttttacttgatgggagccgtatgagctgagaggttc |
| | | acgtacggttctgcgagaggctgctggggtggttccggtggcctactcacctcagttggtt |
| unknown | 187 | tgagattatgtgtgcacagaggattgagcgagagtaaagcacatttaccccacataaattgctgaatcgaactatagata |
| marine | | agatggtggactgtaagccatattttaaagtttatttaaatagaagattaaytgtagaggtcgagcgaacagactggctg |
| bacterium | | agttagtcaataaatagctttaaattacaggtaaaaaaaaacaggattgaaccaccagcagggactgtcaagagtatct |
| | | gctaaaccgccttacggtgcttttatttaacaatagaggtattgagcggctaaggaaaaggggcgatgaaagaatcgttt |
| | | caggtgtttgtacaagagctgaacgaaagtaaactgaccgatgagatgtcgaaactcatttaggcagtgttggaaaccg |
| | | agggaatataatctgcttcgggacaagcacaagggtaacttgaatataccacttgagccaacactcggcgtataggca |
| | | gcaggcttgtatatgggcttgtttatggaactgtggaaaccatcatatgttgtaaattgaaacaacacaagcggtaaaac |
| | | crcgaggttaaaagtagagatacatatgatggtggcggattaagtcgtagtagtgaagaagttcttgtaatgagggtgg |
| | | agcgaaggacttaagcaaacaggagttaacaattgcaacaacttaaaattaaggatgattgaggcgttaagaccaaaa |
| | | acacaaccagtaagtaaagagcaggtatggtctgcgtggaaacgagtaaagcgaggaggaaaaggcatgggagtt |
| | | gaccatgttagtatggaagccatcgcatcgaatccacggaaatacctgtatccattgtggaatagactatcaagtggga |
| | | gttattttcctccacctgttaagttagtgcctattcccaaaggagatggcaaagaaaggatgttgggcataccaaccataa |
| | | tcgacagagtagcacaagaagtcatcaaggcggaattggaagtcatagtcgaaccgaggtttcaccccagttcatttg |
| | | gctatagaccgcacaaaagtgctcatgaagccttggaacaatgtgcaaagaatagttgggaaaggtggtatgtggtag |
| | | atttggacattaaaggattctttgacaacatagaccatgaaaagatgatggggatattacgaaagcataccaacaagaa |
| | | acatatcctactttactgcgataggtggctaaaaacccctatgcaagacagggttggaggggtacaggcaagaatgaa |
| | | aggaacgccacaaggaggtgtaataagccctttgttggcaaatctttatttgcacgaggcttttgaccaatggatatcga |
| | | caacacaacctcgtatagtatttgaaagatatgccgatgatattgttattcatacaagaagtatggagcaatcgcattttata |
| | | ttagacaaattaaaagcacggttgaaaagttattctttagagctacatcccgataagacaaagatagtatattgctatcgc |
| | | actgcgcggtttcacaaagaaggtaaagagatacccgtatcttttgatttccttggttttacttttaaaccacggctttgttta |
| | | aaatcaaacggtgagaaattttggggattccgtcctgcaattagtaaaaagagtgagaaacggatattaggagagttac |
| | | gaaagttaaaaatacatagatggatatatatgaatattcatcaagtatcacaacgcttaagagcaaaggtacgaggttgg |
| | | ctaaattattatggtagatttcgtaaatcagagcttagttggattttcaggcatctaaacatccgaatttcaaaatgggcacg |
| | | gaataaatataagttaaagacttattccaaatcttatgggtggcttaaacgtatggtaaagtggtatccaaatacatctttac |
| | | attgggagcatggctttacaggttaggtttgtttggaagagccgtatgaggggagactttcacgtacggttctgtgagag |
| | | gtttagggtgaaatcccctttacctacttgactcgcttgtgt |
| Crocosphaera | 188 | ttatacttctgtgcgattcgttgagctaaaaagctgacatccaagtacatcacaaggatacagcgtgtagctcattcaaga |
| watsonii | | gccttctaaaggcaattcaatggttaaacgaccaatctaccccaaagatgaaaaacaagagccttaaagctcaattttca |
| | | aggggcagaacacaaaggtcagaagggtaaaaacaatacccgaacggtaaaaaccaaagaaaagtttacaaactta |
| | | agtagtaattaccgaattaactaagtagggataaccgaacagtaaacgaacctagaaacttgaataactgttggtttgttg |
| | | gctaaaggagccatactcggaatcaagggagaaatcccgagagtaacgacataaccctccgttttggacttagtaaac |
| | | ccaggacggaagcagggataaaaggggatacccactgtgaaacagcacaagaaatcctcaagtcaatcggtcacg |
| | | gttaaacattacgttaaatcatcgacttgaaccctcaaaaaaccgaagtagccaaatctgtttgataggctgtgttcaaaa |
| | | gaacaaggtgaaaagtggagtttttgaagtaacatccgcacagattcctaaaagtaacctgggggagagatgaaacct |
| | | aacaccgatatacccaaaccaacggaacgaattaagtccatgattgctcttaccactggtcggtaggtaagtaggaaca |
| | | aacttaaccgcaagggtcatggatgtgagaagcaataagaagctaatgcctacttgtaataagtatggatatgctgacgt |
| | | attgctggtctattagtaagaaatagtctttattagaggtagttaatgtcgaatgcgagtataaccaagactacgccagaat |
| | | ggaacacaatcaactgggcaaaagtccagaggaaagtgtttaagcttcaaaagagaatatttcaagctgttaaatcagg |
| | | taataaagttaaagctaaaaagcttcagaagttactgttaaagtcacactacgcaaagctcttagccatacgcaaagtga |
| | | cccaagacaaccaaggcaagaaaactgccggagttgatgggaaaaaggcactacaccctaaccaaagaataaagtt |
| | | agtcaaagaattaactttaaaaggttacaaagcaaaggcactcagacgggtatggattcccaaccacagaagagatga |
| | | aaaacgtggattggggatacccaccatgaaagatagagtaatgcaagccttggttaaatcagccttagaaccatattgg |
| | | gaagcccaatttgagggaacatcctatggttttcgtccaggcagaagcgcacacgatgctatcaccagaatatacaca |
| | | gcaattaataagaaagctaggtatgtactagatgccgatatcgccaagtgctttgacaagattaaccatgattacctactg |
| | | tctaaagtagattgtccacacaacatcaaaagaatcattaaacaatggttagaatgcggcgttatggacaaaggcatctt |
| | | agaggaaaccgattcgggaacacctcaaggtggtgtaataagcccactattggctaacattgcactacacggaatgat |
| | | aaaagatacagaaaaccacttcccccgtacaaaacgtaaggaagatggaagtctacacagggagtataaacccaaaa |
| | | tcatccgttatgccgatgatttcgtagttctccacgaggattacgacgttattctacaatgtaaaactttaattgcacaatgg |
| | | ctagaaaaggtcggattggaactcaaaccagaaaaaacctccatcagacataccctaaaaagtattgaacatgacggg |
| | | aaaaccattgaccccggtttcgatttccttggattcaacataaggtcataccccgttggaaaacaccactccggtaaaac |
| | | aggaggaaattcaaaatgggggatagaatccaaagcaataggtttcaaaaccttaatcaaaccaagcaagaaaaaga |
| | | tactggcgcatcatgaggcaatcaaagaggttgtaaaagccaacaagaaagccccacaagaagtgttaatcgcaaga |
| | | ttaaaccccataatccgaggatggtgtaattattaccgaactgtaacaagtagagaaacattctcatccgaagattcaata |
| | | ttatggaatacacttagagcctggacagtaaacaggaaaaagaaagagacaccactgtatgatgccctcaaaaaatatt |
| | | tctcatacggaaaacaagggaaatggacattccaaacaagggaatatgtcctctatcaccacaccgaaacggaaatta |
| | | agagacatacactggttaagcctgaatcatcaccctacgacgggaactggacttactggagcaaaagaagaggtaca |
| | | tatactgggacaccagccagggttgcgaaactcttaaagaaacagaaaggtatatgccctcaatgcaagcaacacttc |
| | | acacctgaagacttgatagaagttgaccacatcatcccgaaatcgaaaggtggaaaggatacttacaacaacctacaa |
| | | gctctacatcgtcactgtcacgatgccaaaagcaaaaatgactacctctatgattggcacttataatggctatgaatggaa |
| | | agacgatgtattaacagtaccctagacaagggactaattgccgagagccgtgtgaggtgaaagtctcacgcacggttc |
| | | ggaatgggaggagaggaaggtgacttccttttcgacccctaatcatctatccc |
| Clostridium | 189 | gaaatttaccgcgcgtccagaagcgccgcttgtcatccgcaggcggtgtgcggacattttgggaatccggctttggca |
| cf | | agccggtaaagaaaagtatcaaagacgctgaaaaagtgtcgctttagcgtcagctggtttaccccggagggaaacca |
| | | caagggggacaatagcgtaccggaaacgtcgcaagttgggaaaaccatctacccacgactgagcggcaagacgca |
| | | acattgtgaacaaggaataaggctaaactgctttaagggcagtccgaggcgggatactcgacccggcggcgtaggat |
| | | ggttgtattcgccgtatgtcatagcggaatgtgacaggtatacacagaccgcaggatacttatgacagccagccgcagt |
| | | aagcggaaaaggacgaaaacccactccgacatcacaatacgcttttcccaaagtgtctaactggagattgcctaaacc |
| | | ggaacgccggaaccatccggctatgcgtaatgccgcaaggtgaaaaatttcaaggggtaaaaacctaagaaagatg |
| | | acgctgaatatccggcatggcaacggagccttcgtagtagtccgaggacgggaaagccgtctacatggcgaaggaa |
| | | ggcaggatgtcaaccaactcataaaaaggaaaggtgcgtgaggcattatgagaagtcctgagaatgtgttggaaagc |
| | | ctaaaatccaaggcgtgtaaccagagttacaagtacgagcggttataccgcaatctgtacaatccacaattctatctgct |
| | | ggcataccagcggatacaggcaaaaccaggcaatatgactgccggaacagacggcaagacgattgacggcatggg |
| | | aatggcgagaatcaacgccctcattgaaaaaatgcggaattccagctaccagcccaaccccgcaaggagaacatac |
| | | attccgaaatccaacggaaaaatgcgccccttgggaataccgtcattcgatgacaaactgatacaggaggtggtgcgg |
| | | ctcattctggagagtatctatgagccaaccttcagcgaccactcccacggcttccgcatgaacaaaagctgccatacgg |
| | | cactcaagtatgtgcagaaatatttcacggggacaaaatggtttgttgagggcgacataaagggctgttttgacaatgta |
| | | gaccatcacgtgctgattgccattctgcgaaagcggattgcggacgagcagttcattggtttgctctggaagttcctgaa |
| | | agccggatatatggaggactggaactaccacaatacttactccggtactccgcaaggctctattatcagccccatcctc |
| | | gcaaacatttatctgaacgaattggatcacttcatggcggaatacgcagagaagttcaactgcggagatcgccgtaga |
| | | atcaatccggcgttcaagaagaaattggatgtctgccgggggaaagaagaacggctcaaaagaaacatctccaaaat |
| | | gagcgaggaggaaaaagagggcttgctcgccgaaatcagcgaactgcggcgtagcttgcggtcaatgccgtacag |
| | | cgaccagatggacgaaggctataaaagagttttctatatccggtacgccgatgattttctgattggagtaattggcagaa |
| | | aagcggacgcagaacaggtcaaacaggatgtggggcactttatccgggaaaatctccatctggaaatgtccgaggaa |
| | | aaaactctgattacccacgggcatgactttgccaagttcctgggatatgaggtcacaatcgccaaaggtgagtgtaaca |
| | | aaaagaccagaaccggagccaccagacgagtaaacaacggaaaggtcctgctctatgtcccccacgacaagtggat |
| | | caagcggctactctcctatcatgccctcaaaatcagatacgacaaacagaatggaaacagagaggtttgggaacctgt |
| | | ccggcgcacccgcctgttgcacctggacgatttggaaatcctgaaccagtacaacgcagaaatccgtggattgtacaa |
| | | ctactatcggctcgcaaacaatgtgtctgtcctcaacaacttctactatgtaatgagatacagtatgctcaagaccttttcc |
| | | ggaaaataccggacacgaattagcagaatcattcagaagtaccgtcaagggaaagattttgttgtggagtatccgaag |
| | | aaaaacggccaggttggaaaggtgctgttctacaacaacgggtttcgccgtgataccaaagtggaaagcggcaatcc |
| | | cgatgtgattgcaagagtgtttgagaactacggacgcaatagcctgataaagcggctgaaagcgagtcgatgtgagtg |
| | | gtgcggtgcagagaatgtaccgattgaaatacaccatgtacgaaaactcaaagacttaagcggcaagaaacaatggg |
| | | aaattgccatgattgggcgcaagcgcaagacaatggcactctgcgttgactgtcacgataagttacacgctgggaaatt |
| | | agactgacatctggagagccggatacatcgagaggtgtaagtccggttcggagggaagttctcggaaacctgccata |
| | | gtgatatggcaaggcgctgggttcttaccctacttgtcacgtc |
| Clostridium | 190 | gcttttttccgtgcgcccagcatgggcgcactcttggaggtgcaagtcctcccgtgagttgatcacagcgaacgaagc |
| saccharolyticum | | gaagcgcaactgcatgagggtgaccgagtgtgggaaggaagcgtggagcgcaactgcgagccgatggacaagaa |
| | | ccggatagaaggcggtgccgagcagggcgagcgggcagaaaaccgcgaagctctcgtgatcaaggcgaggtgtc |
| | | gtaaatccggcggttgtgcagtgaaggagtgcgttcttacctggggagatctcgccttgtgcctgaaagggcgacgtc |
| | | gagaggcggagcgagaagtcagccgaggtcgtagtagtcaggggctgggccggtgaggctgaaaccgctggcga |
| | | aggaccgaacgagagggagtgtagaccgacatgtcgatgcggcaggccatgcgtcagatgcccgcgcaagcggg |
| | | gcgggcgggagtagcgcgcggtgaagccgcgcgtgtacccgtcagcgacgaagcctgcggcccgcggcgtgag |
| | | ccggaaaacacagggtcggcgctgctgcaggcagcgctgacgagagagaacctgcaacgggcgctcaaacgggt |
| | | acgtgccaataaaggcgcagcgggcgtcgacggtctggacatcgaccaaacggcacgccatctcgcgacggcctg |
| | | gcccggtatccgtgagcacctgttgcgggggacgtaccggccgagtccggtacgacgggtagcgatccctaagccg |
| | | gacggcggcgagcgcgagcttggcatcccgacggtgacggatcggctgatccagcaggcgctgctacaggtactg |
| | | caaccgatacttgatccgagcttcagcgagcacagccacggcttccggccggggcggcgtgcgcaggacgcggtg |
| | | cttgccgcgcaggcgtacgtgcagtccggccggcgcgtggtggtggatgtggacctggagaagttcttcgaccgggt |
| | | caaccacgacatcctgatcgaccgcctacagaaacgcatcgatgacgccggggtgatccggctggtgcgtgcgtatc |
| | | tgaacagcgggatcatgagggatggcgtggtgcaggagcgcgggcaggggacgccgcaaggcggaccgctgtc |
| | | gccgctgctggccaatgtcttgctcgatgaggtggacaaggccttggagcggcgaggccactgcttcgtgcgctatg |
| | | ccgacgactgcaacgtgtatgtgcgcagtcgccgggcgggagcgcgggtgatggctttgctgcggcgtctgtacgg |
| | | caagttgtgcctgaaggtcaatgaagccaagagcgcggtagccagtgtgttcggccgcaagttcctgggctacagcc |
| | | tgtgggtggcccggggaggaacggtcaagcgcaaggtggcggccaagccgctggcgacgttcaagcagcgggtg |
| | | cggcaactgacccggcgcctggggggcgcagcatgcaggacgtggtgcaaaggctgcgaccttatctgctgggtt |
| | | ggaaagcgtacttcggactggcgcaaacaccgaggatctggcgcgagctggacgaggggttgcgtcatcggctgc |
| | | gtgcgatccagctcaagcagtggaaacgagggccgaccatgttccgggagttgcgcgcgttgggcgcaagccccg |
| | | ccgtggcgcagcgggtggcggccaatagccgctgctggtggcgcaacagcgacaaactgctcaacagcgtgttga |
| | | cggtggcatggttcgaccgcctgggtctaccccgtctgtcctgacctcaacctctcgaaccgcccggtgcggacccg |
| | | catgccgggtggtgtggcaggggcgcagtctacgatgactgtcccctatgccgattccgcacagc |
| Burkholderia | 191 | tacgtacagtgtgcgccgggagaccggtagagatcaaggggtgaaagtccccgacattgcaagaactagcgattca |
| cenocepacia | | cgatttccccgagtcatgcgtggccagccgcgaggcgggttgcgaagcgttgacaggggcgctgataggccagcc |
| | | attgagccacgaaatggatacattcccgagtgctgacgtactactgtatgcggaaggcaacatcgcgcggcgcgctat |
| | | cgcgagcgctgcgcggactcggcgtggtctgagaccctggcatgtcagtacgatctctacgcgggaaccgggagat |
| | | ctcccatctggccgccgacggaaatatggtcgagcggtccgcatcgggaaggcgaggagctgaagccgatgacga |
| | | acggatgggagaagtcggactcgcccatagtagcgaagaagctggcgaacaaacccgggcaaccggatgcggag |
| | | tcagtggagcgaaggggtggggccaaggggaacacggaatggtcgcgcacgcgccggacgcagagccggataa |
| | | gcgtgtcccagagactggaccgtgtgcgtcaagccgcaaggcaaaggaagaaggagaagttcacggcgttgtacc |
| | | acttgatcgacctcgaccttctggcgacggcgttcttctggctgaagcggaaggcggcagccggggtcgatggcgtg |
| | | acgtggcacgactatgagcaagatctcgaccgaaatctcgaagacctgcatggcagactccgccggcaggcttatcg |
| | | ggcgctgcccagtcgcagacggtacataccgaaggcggatggcaaacagcgaccgctgggcattgctgcgctgga |
| | | agacaagatcgtccagcgtgcactggtggcagtactcaatgctgtctacgagatggacttcttgggattctcatacggtt |
| | | tccggccgcaacgcagccagcacgatgcgctggatgcacttgcgacgggaatcgctcgcacgagcgtgagctgga |
| | | ttctggacgccgacatcagcaggttctttgatacggtcgaccacgattggctgatccgtttcgtggaacatcgcataggc |
| | | gaccagcgtgtgatccgactgatccgcaaatggctgaaggcgggagcgatggaagacggggtgatcgagcccaca |
| | | gacgaggggacgccgcaagggtctgtcatctcgcctctgctggcgaacatctacctgcattacgtcttcgacctctgg |
| | | gcaaaccagtggcgcaagcgccacgccgaggggaacgtcgtaatcgtccgatacgccgacgacgtcgtggttggtt |
| | | tcgataaaccacacgatgcgaagcggttcaggcgggccatgcaacaacgactggagcagtttgggctgtcggtaca |
| | | cccggagaaaacccggctgatcgaattcgggcgctttgcggcccggaaccgggcaagccgagggctgggcaaac |
| | | ccgagacgttcaacttccttggattcacccacatcagtggccgtgcgaaagacggtaggttcatgctcatgcgtaagac |
| | | gcggagcgaccgtcttcgaacggcgctgaagggaatcaaggatgagttgcggcacagatggcaccagtcgattccc |
| | | gagcaaggggactggttacgccgcgtggttcaagggtacttcaactaccatgcggtgccgaccaacttcgctgcgttg |
| | | cgagccttccgcgcgcgcgagatcgacctctggaggctggcgcttcggcgccgcagccaaaaggacgacaccac |
| | | gtgggcaaagatgcaccggctcgcgaagcagtggatacccaaggctcgcatccttcacccctggccggtcgtgcgg |
| | | tttgacgccaaccacccgaggcaggagcccggtgcgcgaatcgcgcacgccgggatctgtgcggggggtgccca |
| | | gtaatgggcattcctaccgcgacagtgtagcct |
| Bacteroides | 192 | tcagaagttcgtgcgacgtgaagttgcgtaagtgattgtgtaacatcaagattacacctccagttccgtctggagtagcct |
| vulgatus | | acggacacgtgcattgggagtaatccttttgtacggagctgtcccgacaaggtagcctttccaaaaggagaagattgaa |
| | | ccgtgaggggaaatcaacggctgataacaccattcagcacgaggagcttggcttagcggtatggtcaacgaaagtga |
| | | actgtccgataaacatcgtaaggtataacgagccaaaggctgttgacaggctcggaccaaaaggtaagcagtcggct |
| | | gtcctcttgaactttgggggcacacggacattagcactgccggtggaatagacaggacctaacccgcttatttatcactt |
| | | atgtaaaacacggtaagcccgtatctctccagttcatcagagctggtaagcaagccgcaaggcaagcctacatggggt |
| | | gtgggtataggatagcggaaaaagcgaatgcctccctgtaatggggaggatacggattgagccaatatcacgggttg |
| | | gtaaaggtaacatcatccgacacgaaagtgggcagacttccgcctatggtgacactttacaagatagtttacagaacttt |
| | | taaagaaaggaaagcaaatgaacgcaaagaaattggcgtgtgcgcccgaagacatagacattcacgaactctggtcg |
| | | aaaatcgactgggacaaatgtgagcgttttgtccaaaagctgcaagcacgtattgtaaaggctcaaagggaaggcag |
| | | aaataacaaggtgaaagccctgcaatggatgcttacccactcgttctacgccaaagcattggcggtaaaacgagttac |
| | | gactaacaaaggaaaatctacttccggcgttgacaagattacatggtcttctccattggcgaaagccaaagccatattca |
| | | ctctgaaaagacatggctacaagccacaacctttaaaacgtgtaaatatcaaaaagaagaacgggaaattacgcccac |
| | | tcggaataccaacgatgaaagaccgtgcgatgcaggcattgtacctaatggcacttgacccgatagcggaaaccact |
| | | ggcgacagccattcctacggattccgcagacatcgctgtacacatgatgccatcgagcaatgctacatcgttctttctcg |
| | | ttcggtcgctcccgaatggattcttgagggggacatcaaaggatgcttcgaccatatcagccatgcgtggttaatcaac |
| | | aatatccctatggataaggagatactacgcaaatggctcgaatgcggatatgtgttcaatggagaactctttcctacgga |
| | | agagggaaccccacaaggtggcataatatccccaactcttgcaaacatggcacttgacggactgcaagatttgttgga |
| | | aaagagtgtcaagaaatatcaggtgaactacaaaaagatagttcctaagatacatcttgtacgatatgcagatgatttcat |
| | | tgtaacggcaaaggataaggaaacgatagaacaggttatacttccattagtacgtaagttccttgcggaaagaggtctg |
| | | acactatcagaagaaaagacaaaaatcacacatataaatgagggatttgactttctcggtttcaatatccgaaagttccg |
| | | caataatacgctgctgacaactccatccaaagatgcgcaaaagaggttttgcgaaaaaatccgcaagacgatagaag |
| | | ccaacaagtgtgtaaaacagaagtcacttattatgatgctgaatccaatcataaaaggttggggtaattattataaatatgg |
| | | tacttctgcgaatgtattccaccgaatggattgggaaatcttcaagaaaatatggcaatgggcaagacgcagacatccg |
| | | cagaaatgtaaaggatgggtaaaggataagtatttcagaacactaaatgggcactcgtggagatttgcggcagatatg |
| | | ggcaagaaagataagattgattatctcgaactgacctatctaccaaccatccaccatgagaaattcgtcaaggttcggc |
| | | attatgccaatccgtatgacccatcagataaatcgtactacgaatggagagaaacctaccgaatgaagcaaacgctga |
| | | aaggcaggcaatctctaataaacatttggaaacgacagaataaagtatgtcctgtatgtggagagcgtattgacaggga |
| | | aagaccttggagtatcactgaacaaatagtcagtggacgaaaggtaagaactcttgtacataccagttgtaaacgtaaa |
| | | atgcaaagtagattatgaatatgaaagtcgaaaatgagatggctctaccgataacaaagtcggtaacggtcatgttgctt |
| | | gagccgtatgcggggaaactcgcacgtacggttcttagaggggaaagctcccgtaagggggctgacctactcgaca |
| | | gttcgggcg |
| Chlorobium | 193 | tcaatttagggtgcgccgggagaccggtaagaggtaaagggtgcaagtccctgcagtgaaaggattagcgatccgc |
| phaeobacteroides | | actggccccgagtcatgcgcgcataaccgcgaggatatgcgcgaagcattgacaggggtgcatataggccagccat |
| | | tgagcggcgaaaatgagcaaatttggggagcccatgccctcctaaaagcagaaggcaatacagacgaagtgcgata |
| | | acgcgagcacaagactgtccccacgacgtcagagaacctggcatgtatgtaagcctcttacacgggaaccgggaga |
| | | tctccgataccacccatcgtcgttggcgggtcgcatcgggaaaacgtaatagttgaagccgatgatgtacggtacgga |
| | | gaagtcagacctgcccatactaccggagaagcgagcgaacaaagcgggtaagcccgtagcggagttcgtggaggg |
| | | aaggggtgggaacaagagaaatgcggaactgcaaagcatggcccggacacagggtcgggaagccgtgttccaag |
| | | cgcagggtcgcatacgtgaagcagtaaccagaaatcgaggagaaaagctgacagcgcttctgcatcacatctccgta |
| | | gactgcctgcgctggtcgtacttcgaactgaagaagaccgcagcaacgggagtcgatgggataacgtggaaagatta |
| | | cggggaaggactggaagagaaccttgcagacctgcaccggcgaatccacacgggagcgtaccgggcacagccat |
| | | cgcgccggaagtacataccgaaggcaaatggccaacaacgaccgctcggcatcgccgcattggaagacaaaattgt |
| | | acagcgggcagtggtggcgattctcacgccaatctacgaagcggagtttctggggttcagctatggattccgtccggg |
| | | acgcagtcagcatgatgcgctggatgcactcgcatacgggatcaaggtgaagaaaatcggctgggtattggacgccg |
| | | atatttcccggttttttgatacgatcagccatgagtggatgatacgctttttggaacaccgtatcggtgacaagcggattgt |
| | | ccgactgattatcaagtggctgaaagctggggtactggaagatagcgtacggatcgaggcagaagaaggaacaccg |
| | | cagggagccgtgatatcaccgctccttgcgaacatctaccttcactatgcttatgacctgtgggctaagcagtggcggg |
| | | aaaagcactgcaaaggtgatatgatcgtcgtgcgtttcgctgacgatagtgtcgcaggcttccagaacaaggaagatg |
| | | gcgaaagattcctagctgatctgaaagagcgactggcaaagttcgcgctaacgttgcatccagagaaaacccgactg |
| | | attgagttcggcaggtatgctgccaagaaccgacaaagacggggacaagggcggccggaaacctttgactttctcgg |
| | | atttacccacatctgtggagagaaagtagtggggaaaggctttcagctcttgcgcaagacgaagcgcggatcagtga |
| | | gaaccaagctcggagaggtaaagaaagagctgagacggcgaatgcatgtctcagtatcggaacagggaaagtggc |
| | | tgaacagcgtgttgagaggtcactatgcctatttcgcagtgcccacaaacacgagggcgttatcggcattccgctacca |
| | | tgttgccagacgttggatgaagagcctcaggcgccgaagccagcggcatgtgatgacatgggagcgaatgatgatat |
| | | atattgatcagtacctcccaaacccaaagatccttcacccatggccggaacaacggttttgcgtcaaacactcaagttag |
| | | gagccggatgcgggaattccgcttgtccggatctgtccggggggtgaggggtaactcttgtccctaccgggattttaaa |
| | | tatg |
| Coprococcus | 194 | aatcggaattgtgcgcccagataagggcgagtagtctagcaggtggaatgcctgtctggtaaggtctaaccaaccgcc |
| catus | | agtagcgagtcttgggtgactgtcagtaacggtagttgctaagcgtagacagcgagaaaatagagagtgtgattgagc |
| | | ctcgaaattttgacatttagaaggctgacgttctaatttcgacggaaagcaatacaaatcaattcgcattggtgagaaatg |
| | | gtttgcttctgcggggtcaaagagcactttatgttttacattgtgattattcagtcaactggggagagcctattgtttcttgcc |
| | | attgcaagtatggcaaacaactgcaaaacaggagaatgtcaaaagaacagtaggcagtcggatagcttcatagtactg |
| | | aagaagttgggtaatgccaacggaggaaagggagctacataataatggtctttctgaggacacatcaaccgtactcag |
| | | ggacggaggtattgatggaaacgaaattagaaagaatagcagaaatatcggcaaattcgccgagaccagagttcaca |
| | | tcgttgtatcatctaatcaataaagaaatgcttttgcaatgtcacaaagaattagacggaaacaaagcagtcggtgttgat |
| | | gagattactaagaaagagtatgagagaaatctggagcaaaacatagatgacttggtagaaagactgaaaagaaaatc |
| | | atacaaaccacagccatcaattagggtatatatccctaaaagcaatggaaaacttcgaccattaggaattgcgtgctatg |
| | | aggacaaaatagtccaattagcattaaagaagatattggaagctatctatgaaccgagatttctgaactgcatgtacgga |
| | | ttcagaccaaaccgaggatgtcataacgcaataaaagaactgtataagagattaaacaacacgaaaatctgctatattgt |
| | | ggatgctgacatcaaaggattcttcgaccatatgaagcatgaatggattatcaagtttctcaaactttatattaaagacccc |
| | | aatataattggtcttgtcaagaagtatcttaaagtgggagtgatggataatggtgaacttatggtgaatgaagaaggttca |
| | | gctcaaggcaatatcataagtccaatcctagcaaacatatatatgcacaatgtattgacactatggtataagtttattattac |
| | | caaagaatgcaagggagataactttctaattgcatatgcagatgattttgttgcaggatttcaatgcaaatgggaagcag |
| | | agaattactacaagcttcttaaagaacgaatggaaaagttcggtttacaattagaagatagtaaaagtcgtctcttacaaa |
| | | gcggtgcttatattgcaagggcaaagcaaaagagcggtgagtgtatcagattacaaacatttgattttctgggattcacat |
| | | tctattgtgggcgttcacgcaaaggcatgccatatataatgccaaagacaagctctaagaaattcagacagaaaattcg |
| | | aggtatcaaggtatggttatatgcgaatagagaccaaccgctaaagaaactaatgggtatgctaaatctgaaactaata |
| | | ggtcattataggtactatggcataagttttaatggcagaatgatttcaaactacaaacaacaagtgagagaacttctgttta |
| | | aggtgctgaatagacgaagtgacagaaagagttatacaagagaaggattcattgaaatgctgaaatattatccattggc |
| | | aatgccaaagatttatgtcagcttgttttgagaatgtaaatattattatgaagagccgtatgcgggaaagccgcacgtacg |
| | | gttctgtgaggggcttacattgtgaggtgtaagtctactcgactgaggtgatg |
| Cupriavidus | 195 | ttttctttttgtgcgcccggcagggcgcactcacttgagggtgaaagtcccttacacacccggcaaggggaagtgttag |
| taiwanensis | | ccataggcaagggttccccgggcgactgggggtctgaaggaagcccgaggcaaagcgctggcctgacgaacagg |
| | | aagcggatatgaggcgacgcatctgggtaaggcagcaaaaagggccaaagcccagtacttgcacggaacggtgcg |
| | | gcgtagatccgacaggcataagcgtgaaggtgggtgcgtcatacccggggagatctgtaccgctgccacggtgcta |
| | | ccggcgtcgagaggtgtcgggatggcggtgcagaagtcagcagaggccatattaggcgagccgttggcgctgaag |
| | | ggccgaacatgaatgagcgcgattaggacgacagacctcgatgcttaccgacgaagcccaaatgcacgaacgagtg |
| | | cagcccacagcggaggaaggcgggcggaacctgcccggggccgatgggggtgcggaggctggcacggcggct |
| | | gttgggcaaacgaaagcgcgggcgccatcgctgatggaggcggtggtcgagagaagcaacatgtggctggcgtac |
| | | cggagggtggtcggcaatggcggtgcggccggggtggatgccttggaggtgacggcgttgcgtgactggctgaag |
| | | gtgagctggccaagcgtcagggcggcgttgctgggtggtcagtacatcccgcagtcggtgcgcgcggtggacattc |
| | | ccaagccttcgggggggtgaggacactgggcatcccgacggtggtggaccggctgatccagcaggcgctgctgc |
| | | aggttctccaaccgctctacgaaccggggttctccgagtcgagctatggcttcaggccgaggcgcagcgcccagca |
| | | ggcggtcttgcaggcacagcggtacgtgcaggaaggccggcgatgggtggtggacatcgaccttgagaagttcttc |
| | | gaccgggtcaaccacgacatcctgatgtcgcgggtggcacggcaagtgaaggatgtccgggtgctcaagctgatcc |
| | | ggcggtatctggaagcggggctaatgcgtggcggggtggtcgaggcgaggaggcagggcacgccgcagggcgg |
| | | gccgctgtcgccgctgctgtcgaacattctgctgacggattgggaccgtgaactcgagaagcgggggttggcgttctg |
| | | tcgctacgcggacgactgcaatatctacgtccgaagccaggcagcaggacagaggctgttggccgggatgatgacg |
| | | ttccttgcggagcggctgaatctgcaggtcaatgaggccaagagtgcctgtgcgcggccttgggcacgcaagttcctg |
| | | ggctacagcctgacagcgcatcgtcaggccaagctgcgtattgccccggaaagtctgcaaagactgaccggacgaa |
| | | tcaaggaactgatgcgcaaagggcgaggaaggagcctggctcacacgatagcggtgctgaatccggtgctgcgcg |
| | | ggtggatcgggtacttccagcacacccagagcaagcggccattggaggagctggacgggtggatacggcgccggc |
| | | tgcggtgcttgctatggcggcaggccaagagccgtgctacccgcactgtgatgctgcgtcggcaaggcttaacggag |
| | | gatcgcgcgtggcactcggctcgcaacgggcagggcccatggtggaatgccggggctcgccacatgggcgcggc |
| | | cttcccgaagggctacttcgacgccttggggttgatctcgctgctggatactcagcagcgcttgcagtctcgttcgtgaa |
| | | ccgccgtatgcggaaccgcacgtacggtggtgtgagagggctgagggggtgacccctcaccctactcaatcctgcc |
| | | gcac |
| Chlorobium | 196 | atgacatggagtgcgacgtgaagtcgcgtatttgaactgttctttgcagaaccagttgtgccgtcaactgcccccaccg |
| luteolum | | gcacatgcaggatgagcaatcgccctgtgtgaagctaccggaaacgtacccgcgggaaaccgtaggccgacccgt |
| | | gaggagaagctgaatctgccagtagcaggcggagaggggcaagggggaggatggtatggttaacgcaggtgaatc |
| | | gtcacaaccatcgttataataagcaagcgaaagctcactgacaacgcttggaccaaaaggtacgcgggcaggtatgc |
| | | tcccttccatgtggagcacacacggccaacaaatcccgccggtggagagacggaacctaacccatccgcatgttctat |
| | | acgcggaacacggaaaccccgtacttccgcccggccatgccgggcaatccgactgtaaggaaggaccatgaaagt |
| | | gcgggtatgagagtggggaagaagcgaacgccgtcctgtaatcgggcggataggggttgcaacatcaccccacgt |
| | | gaaaacgggctgacgtcctcatggtctcgatttgcaagataacttgaagaacctcccatgcagaaggaacgcaaatga |
| | | acacggataaccctgtgtgtgcaccttccggcctcgactggcaagggatcaactggtcccggatcaagcgacaggtc |
| | | aggaggcttcaggcacgtattgcaaaggcaacaaaggaaggccgtcatggcagggtgaaagccttgcagtggctgc |
| | | tgacccactcgcacagcggcaaggttcttgccgtcaaacgggtgacggaaaaccgggggaaaaacaccccgggag |
| | | tcgacggagacgtctggaaaacatcaaaagcaaaggccaatgccgcagcatcgctgagacgaagaggctacaagc |
| | | cccttccccttcgaaggacctacattccgaagaagaacggtaagcaacgacctctcggcatcccaaccatgaaagac |
| | | agggccatgcaagcgctctactggcttgcactggagcctgtagcggaaaccaccgcggacggcaactcgtacgggt |
| | | tccgaccatggcggtcaacagccgacgtggcagaacagtgtttcatctgcctcgcaagacgcgattccgcacaatgg |
| | | atacttgaggccgatattgccgggtgcttcgatgctatcagccaccaatggctggtcgacaacatccccatggatacgc |
| | | cgatcctccgcaaatggctgaaggcaggcttcgtgttcaacaacgagctctttcccacagcctcgggcacgccgcaa |
| | | ggcggcatcatcagtccgggactggcaaacatgagccttgacggcctcgagcaagcgcttgcaacagcatttccgca |
| | | agcgcgaagacgaggactgaaaatgcatatggtgcgctacgctgacgacttcatcatcactggaaactcgaaagagt |
| | | ggctggaacatgagatcatgccggtagtggtcgacttccttaaaaaacgagggctctggctctcagaggaaaagacc |
| | | agggtaacgcacattacggaaggatttgatttcctcggctggaacatgcgcaagtacgacggcaagctacttatcaag |
| | | ccctcgaaagcgaacatcaaggcccacctcaccaaggtgggggaatcatcaaggcgaaaaagacgatcaagcag |
| | | gtggatctcatcggtctcctcaaccccgtcctgcggggatgggcgaactaccaccggcacagtgtcgccaaagacgt |
| | | cttcgctcgcaacgaccacgaggtctggtcaatgctctggaaatggggaaacgacgacacccaaataaaggccttc |
| | | gatggatcatggacaaatactttcatgccagaggaggcaggaaatgggtgttcgtcgccgaagaggcggaccgaaa |
| | | gaaagaacgacggctctttctggaagccagcatgccaatccagcgacacgtcaaaatcagaacgaaggcaaacccg |
| | | cacgatccagtgtggagggagtacttcagcgcccgtagaacacagatgtggaaagcgacaccattgagttgccgggt |
| | | gccttacggcgccttattcgaggcttgagccgtgtgcggtgaaagtcgcacgcacggttcttaggggacggtggcgc |
| | | agtaatgcgccgctgttacccgattatggaaaac |
| Chlorobium | 197 | caacccgttcgtgcgacgtgaaagtcgcatagttgaactgttctttgaaccagttgtgccgtcaactgctcccaccggca |
| phaeobacteroides | | catgcaggatgagtaatcgccctgtgtgaagctgccggaaacgtacccgcaggaaaccgtaggctgctccgcgagg |
| | | aaatgccgaatctgccagtagcaggcggagaggggcaagggagagagtggtcttggctaaggtatctgaatcgctg |
| | | caaccgtcgttatgtcgaacgagtgaaagctactgtaacactcgaaccaaaaaggtatgcgagtaggtaaacgcctgg |
| | | atggagcgtttacgcggccaattactctcgccggcggctaggcggagcctacaccacttgagagttcactatgcggaa |
| | | cacggtaagcccgtagtgtcgcccagcatactgggcaatcgaaccgtaaggaacgaccatgatactgtgggtaaagg |
| | | atcgaggagaaagcgaacgcctccctgtaatcgggcggataagggctacaatatcgccctatgcgaaagcaggcag |
| | | acttcctcggggtctcgctttacaagaaaacttgaagaacctcttatgcagaaggaattgcaaatgaacatgggtagtac |
| | | cgtgtgtgcaccttccgcctccaactggcaagggatcgaatggtctcaggtcaaacggcaggtcaagaggcttcagg |
| | | cccgtattgtaaaggcaacacaggaaggtcgccactgcaaagtaaaagcattgcagtggttactgactcactcgtttag |
| | | cggcaaagctcttgccgtgaagcgtgtgacggaaaaccggggaaaacacaccccgggagttgataaccaaatctgg |
| | | ataacgccaaaagcaaaaaccaatgctgtagcatcactaaagcgaaggggttacaagcccctccctcttcgaaggatc |
| | | aatattccgaagaagaacggtaagacccgacctctcggcatcccgacaatgaaagacagggccatgcaggcacttta |
| | | tctgcttgcactggaacctgtagcggaaaccactgcggacgataactcgtacgggtttcggccatggcggtcaacagc |
| | | agacgcgagtgcaagatgttttacctgcctcgctcaacgcaattccgcccaatgggtacttgaggctgatattgccagtt |
| | | gcttcgatgctataagccatgagtggttgatcgacaacattccagttgacactgcgatcctccgcctgtggctaaaagca |
| | | ggatttgtgctaaagaacgagctctttcccacagaagccggtacgccgcaaggcggaatcatcagcccggttctggc |
| | | aaacatgtgtcttgacggtctcgagaaggcgctggcgaaagcctttccgcaagcaaagaaacgcggcctgaaaatgc |
| | | acatggtgcgatacgctgacgactttgttataacaggaaactcgaaagagttgctggaaaacgaggtcttgccagtggt |
| | | ggtcgaattcttggctgaaagagggttattcctctctccggaaaagactaagataacgcacattacggaaggctttgattt |
| | | cctcggctggaatgtacgcaagtacagcggcaaactgcttatcaagccgtcgaaagtgaataccaaggcccacctcct |
| | | gaaggtgcgggaaatcattaaaggtaataaaacggccaaacaggtaagcttgatctacctcctcaatcccataatgag |
| | | gggatgggcgaactaccatcaacatgtggtcgccaagaaatcctttgctcgcaatgacgcagaaatctggtcaatgct |
| | | ctggaaatgggcaaaaagacgacacccaaacaaaggaatgcgatgggtcaaggcacggtacttcaagacccaaaa |
| | | cgcccgcaactgggtgtttgccgctaaggatgaggccacaggcaaggaaatcagacttgtcactgaagccgatacac |
| | | caatcaagcgacatgtcaaaattaagtcaaaggcgaaccctcacaatccagtgtgggccgaatactttgcagcccgca |
| | | acaaacagatgaagaaatcagcaccgatgtacagccgggcgtcacaaggcgccttatgcgaggcttgagcccagtg |
| | | cggtgaaagtcgcacgctgggttcttaggggacggtggcgtagcaatgcgccgccgttacccgacatcgagtcgt |
| Geobacter | 198 | tgtttattcggtacgccaggccaagcctggctgatctatctgactgaaaagtgtcagacatgtaaattgctcgttcaccat |
| uraniireducens | | gtagtcgcggtctgtcacgagagggtaaccaaacgtggggagtaagaccggttctttgtaagaaaagagcgaagggt |
| | | acgataactaccctgacgacgaagacgcgagcccagcgcaagcgaacccgtttcggcctcgttacacgtagttgaca |
| | | gtggccaaccttccgaatgtggtgaagcctgccatccaccggtgataaacgagcgaaagaaccggtgggactgtcc |
| | | ggggtgattggggagggcgcgtgtcgaaagatcagccgaggaacctgggaggtccgacgtaattcccaaagcgaa |
| | | agggtttcgccgaagggagaacgccgacggggaatcaataacccgccaagcgtgtacggtcggaattcggagcgg |
| | | cccatagtagccgggaagcggggtaatgcccgtggagccaaggggccgtacttcagtcatgtttacacggaaagag |
| | | aggagacccgcttgagcgacagacgctccataacggaatggatggccgaaggattcgaacctgaacccggactgc |
| | | cggtaaaagtctcccttctgcggtggaaactgagcagcaaggccaaacgagaaccgcagttccggttctatgcccttt |
| | | acgaccgaatccatcgtcgtgatgtacttgaaacggcgtggttgcgagtaaaggcgaacaagggcgcgccgggagt |
| | | cgatggtgtcagcattgaatccatcgaggtgcgggcagacggaatatccggctatcttgacgaaattcaagaaagcct |
| | | gagaaccaaaaactacaaaccatccccggtgcgtcgcgtctacatcaccaagcccaacggcaaactgcgcccgctc |
| | | ggcataccgtgcgtgcgggacagaattgtacaggcagccgtactccttatcctcgaaccgatatttgaggtggactttct |
| | | cgactgctcccatggatttcgtcccaaaaggcgtccccatggagccctcgaccaagtcggtaataacctccaactcgg |
| | | acggcaggaagtgtatgacgcggatctttcgagctactttgacagcatcccccacgagcacctgatagtagaacttga |
| | | gcggcgcatagccgaccggagtgtcctcaaactgatccggcaatggcttcacagtccggtcagagaagaagacggc |
| | | agcatcagccgtcccaaacaagggacgccacaaggcggggtaatatctccccttcttgccaacatctaccttcaccgg |
| | | ctcgaccgggcctttcacgaagaagcagacagcccctaccacttcgccagagcgcgcatggtacggtttgccgacg |
| | | acttcgtagtaatggccagacacatgggcaatcgcataaccggatggctggaagagaaactcgaaacagaccttgga |
| | | ctcagcatcaaccgtgacaaaaccggtattgtcagaatgaacaagaaagagagcctgaactttctgggattcaccctcc |
| | | gctatgaccgtgacctgcggggcagagattgggattatctcaacatcatgccgagtaagaaggccataggagcactc |
| | | aagggaaaaatccgggagaagacccgaagcggatacaaaaagccgcttgtcgaagcaattgccgaggtaaacagc |
| | | atccttcgcggatggggcaactacttcgactacgggtatccccggaaagtatttcgagacgtgaatcattacacgagga |
| | | ctcgctttcgaatatttctgaacaatcggagccaaaggcggagcaaacccttcagggcaggtgaaagcctgtatgccg |
| | | gacttaagcgatatggtctggtttacctgtaacagcaaagccacaactcctcaataatgccgtgcagcaagaggctgat |
| | | aggtaagccgtatgcgggaaatccgcacgtacggtttgacgagggggagctggttaaggcaactatggtaaggctac |
| | | ttaggcaccgccaaacgaaaggggcggaaacggacaagccaaacctaatgatgtcgtaacctgcttcctactctactg |
| | | gttaattg |
| Geobacillus | 199 | ctcttttcatgtgcgcccggcatgggtgtagtctatagggtgaaagtcccgaactgcgaaggcagaagtagcagttagc |
| sp | | ttaacgcaagggtgtctgcggcgacgcagaatctgaaggaagcgggggcaaacttccggtctgaggaacacgaa |
| | | cttcataggaggctgggtatcattgggtgagtttgcatgacaaaacgaagccctttctgccgaaggtgataccgagtaa |
| | | atgaagcagatagatggaaggaaagactgcactcttacccggggaggtctgtccgggaagccaagtgcgcttggca |
| | | accgttggagcgatccaacgctgaacggacagaagtcagcagaggtcatagtactcgtctagcttaagatagaaggg |
| | | gaaggaccgaaccatgaaggagaacggccactaggcgttcatcttctttgatgaaacagacaacccgaaagggcct |
| | | gcttgagggaggaaatggtgaagtccatgggggacctcaagagggtggagaagaagatggcacgaatagaacgg |
| | | atcgttcacgtagagaggaagaattggaatgtggatgaaacagatactgtcacgggagaatctcctgcgagcactcaa |
| | | acaagtggaaaagaataaagggtcccatggaaccgatggaatgtccgtcaaagacctgcgaagacacctcgtggaa |
| | | cattgggacgtgatacggcgtgctttggaagaagggacctacgaaccttgcccggtccgacgggtcgaaatcccgaa |
| | | accgaacggaggagtcaggttactaggaatcccgaccgtgacagaccggttcatccaacaggccatcgcccaagtg |
| | | ctcacgccgatctttgacccatccttttcggaacacagctacgggtttcgtcccggtcgaagaggacacgacgcggtg |
| | | aaaaaggcgaagcagtatattcaggaaggatatacatgggtggtagatatcgacttggaaaagttctttgatcgagtca |
| | | accatgacaaactgatggggatattagcgaaacgaattccagacaaaatcctcctaaagttgatacggaagtatttaca |
| | | ggcaggggtcatgatcaacggggtggtcatggaaacacaagaggggactccacaaggagggccgctcagtccact |
| | | tttgtccaacattctcttggatgagctggacaaagaattggaaaaacgagggcacaaatttgtacggtatgcggatgact |
| | | gcaatatctacgtaaggacgaagaaggccggggaacgggtgatgaaatcgatcacggcattcattgaaaagaaactc |
| | | cggctgaaagtcaacgaaaccaaatcggcagtggatcgaccgtggaggagaaaattcctcggttttagcttcacccca |
| | | aataaggagccaaaaatccggatcgcaaaggaaagtattcggcgcatgaagcaaaggatgcgcaccatgacgagtc |
| | | gatcgaaaccgattcccatgctcgagcgaatcgaacagctcaaccagtacattctgggatggtgtggatacttctcgtta |
| | | gcagagactccaagtgtgttcaaagaactagatggatggattcaacgaaggctgcgcatgtgccaatggaaagagtg |
| | | gaaacttccgagaaccagagtccgaaaactgcaaagtttaggagtgcccaagcggaaagcatatgaatggggaaac |
| | | actcggaagaaatattggagagtggccgctagtcccatcttgcataaagcccttggcaactcctattgggagagccaa |
| | | gggctgaagagtctttatcaacgatatgaatctctgcgtcagacttaatggaaccgccgtataccgaacggtacgtacg |
| | | gtggtgtgagaggacgagggttaatcaccctctcctactcgatcattctgttg |

In some embodiments, the 3′ or 5′ intron segments and/or 3′ or 5′ exon segments are derived from a gene selected from a species selected from: Cyanobacterium Anabaena sp., T4 phage, Coxiella burnetii, Azoarcus, Tetrahymena thermophila, and Staphylococcus phage Twort. In some embodiments, the 3′ or 5′ intron segment and/or 3′ or 5′ exon segment are developed from permuting at a position along a Cyanobacterium Anabaena sp., T4 phage, Coxiella burnetii, Azoarcus, Tetrahymena thermophila, and Staphylococcus phage Twort intron and/or exon sequence. In some embodiments, the 5′ or 3 monotron element are derived from a gene selected from a species selected from: Cyanobacterium Anabaena sp., T4 phage, Coxiella burnetii, Azoarcus, Tetrahymena thermophila, and Staphylococcus phage Twort. In some embodiments, the 3′ or 5′ intron segment and/or 3′ or 5′ exon segment are developed from permuting at a position along a Cyanobacterium Anabaena sp., T4 phage, Coxiella burnetii, Azoarcus, Tetrahymena thermophila, and Staphylococcus phage Twort intron and/or exon sequence.

In some embodiments, the intron segments and/or exon segments of a provided polynucleotide are derived from a gene from the same species (e.g., a polynucleotide comprises Azoarcus 3′ and 5′ exon segments and Azoarcus 3′ and 5′ intron segments). In other embodiments, the 3′ or 5′ intron segments or 3′ or 5′ exon segments of a provided polynucleotide are derived from genes of different species (e.g., a polynucleotide comprises an Anabaena intron segment and Staphylococcus phage Twort exon segment). In certain embodiments, the monotron element of a provided polynucleotide is derived from a gene of a different species than the 3′ or 5′ intron segments and/or 3′ or 5′ exon segments (e.g., a polynucleotide comprises a Staphylococcus phage Twort montron element and an Anabaena intron segment). In some embodiments, use of genes of one species of an intron segment and/or exon segment may allow for more efficient or effective circularization or self-splicing of one or more polynucleotides as compared to another gene of a different species. In certain embodiments, the gene used of one species develop an intron segment may more efficiently promote the interaction between an intron segment and a nucleophile (e.g., form a more efficient or effective binding pocket that promotes the transesterification reaction of a splice site nucleotide) as compared to an intron segment developed from a gene of a different species. In some embodiments, the gene of one species from which an intron segment is derived may be more efficient in forming a binding pocket for a nucleophile as compared to a different gene of the same species. In some embodiments, the species of gene from which the intron segment is derived may be more efficient in forming a binding pocket for a nucleophile as compared to a species of genes comprising the same and/or homologous sequence from a different species.

As described herein, in some embodiments, a provided polynucleotide comprises an intron segment and/or exon segment derived from permuting at a position along a Group I or Group II gene selected from Table 2 or Table 3. Location or position of the permutation sites may enhance the ability of an intron segment to effectively splice and/or circularize in a provided polynucleotide. In some embodiments, the Group I or Group II genes are permuted at a position that enhances splicing or circularization activity of an intron segment of a provided polynucleotide as compared to a different permutation site. In certain embodiments, the Group I or II genes are permuted at a position in an intron segment of a provided polynucleotide that enhances the provided polynucleotide's ability to self-circularize as compared to a different permutation site. In some embodiments, the Group I or II genes are permuted at a position that enhances or promotes the splicing activity of an intron segment to another intron segment, monotron element and/or exon segment. In some embodiments, the Group I or II genes are permuted at a position that allows the intron segment to more efficiently splice or self-splice than an intron segment permuted at a different position. In certain embodiments, a position of a permutation site may promote the interaction between an intron segment and a nucleophile (e.g., form a more efficient or effective binding pocket that promotes the transesterification reaction of a splice site nucleotide).

As described herein, permutation sites positions are described to mean that the permutation of the natural or synthetic intron occurs at the junction between the listed amino acid and the adjacent downstream amino acid (e.g., an Anabaena position 189 permutation site corresponds herein to a permutation site between amino acids 189 and 190).

In some embodiments, a provided polynucleotide comprises an intron segment derived from permuting at a position along a gene selected from a species selected from: Cyanobacterium Anabaena sp., T4 phage, Coxiella burnetii, Azoarcus, Tetrahymena thermophila, and Staphylococcus phage Twort. In certain embodiments, a provided polynucleotide comprises an intron segment derived from permuting a Cyanobacterium Anabaena sp. gene. In these embodiments, the permutation site of the Cyanobacterium Anabaena sp. gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, or 265 of the Cyanobacterium Anabaena sp. gene. In certain embodiments, a provided polynucleotide comprises an intron segment derived from permuting an Azoarcus gene. In these embodiments, the permutation site of the Azoarcus gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, or 221 of the Azoarcus gene. In certain embodiments, a provided polynucleotide comprises an intron segment derived from permuting an Coxiella burnetii gene. In these embodiments, the permutation site of the Coxiella burnetii gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, or 390 of the Coxiella burnetii gene. In certain embodiments, a provided polynucleotide comprises an intron segment derived from permuting a Tetrahymena thermophila gene. In these embodiments, the permutation site of the Tetrahymena thermophila gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, or 436 of a Tetrahymena thermophila gene. In certain embodiments, a provided polynucleotide comprises an intron segment derived from permuting an T4 phage (td) gene. In these embodiments, the permutation site of the T4 phage (td) gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, or 289 of the T4 phage (td) gene. In certain embodiments, a provided polynucleotide comprises an intron segment derived from permuting a Staphylococcus phage Twort gene. In these embodiments, the permutation site of the Staphylococcus phage Twort gene may be downstream relative to amino acid positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, or 281 of the Staphylococcus phage Twort gene.

Also provided herein are methods of identifying an exon and/or intron element or identifying a combined accessory element comprising a mutated Group I or Group II exon and/or intron segment (as described herein) that allows production of a circular RNA that is translatable or biologically active inside a eukaryotic cell. In some embodiments, such a method comprises:
(i) inserting 5′ and 3′ Group I or Group II intronic sequences derived from a database of native intronic sequence into a precursor RNA polynucleotide;
(ii) transcribing the polynucleotide into RNA in vitro or allowing the polynucleotide to be transcribed into RNA by a cell; and
(iii) determining the circularization efficiency of the RNA produced by the polynucleotide by identifying the amount of circularized RNA, the amount of excised intronic sequences, the amount of precursor RNA remaining after circularization, and combinations thereof.

In some embodiments, the mutated Group I or Group II exon and/or intron element or segment comprises a deletion, insertion or substitution of at least one nucleotide, including but not limited to a nucleotide substitution of one or both the dinucleotides of the 5′ and/or 3′ Group I splice site dinucleotides. In some embodiments, the 5′ or 3′ Group I or Group II intronic sequences, or combinations thereof are sequenced. In some embodiments, the method further comprises comparing the circularization efficiency of the polynucleotide with a polynucleotide comprising a native intronic sequence, or a parent polynucleotide.

Also provided herein are methods of identifying or determining a polynucleotide sequence that improves RNA circularization efficiency compared to a polynucleotide comprising a native intronic sequence or to a parent polynucleotide with a known sequence, the method comprising modifying a DNA sequence encoding the precursor RNA polynucleotide described herein comprising:
(i) modifying at least one nucleotide and/or altering the length of the 5′ intron element and/or 3′ intron element of the DNA sequence encoding the precursor RNA polynucleotide described herein;
(ii) altering the length of the 5′ and/or 3′ internal and/or external spacer sequence of the DNA sequence encoding precursor RNA polynucleotide;
(iii) altering the length of the 5′ and/or 3′ internal duplex sequence of the DNA sequence encoding the precursor RNA polynucleotide;
(iv) altering the length of the 5′ and/or 3′ exon sequence of the DNA sequence encoding the precursor RNA polynucleotide; or
(v) combinations thereof; and transcribing the polynucleotide comprising the DNA sequence into RNA in vitro or allowing the polynucleotide comprising the DNA sequence to be transcribed into RNA by a cell; and determining the circularization efficiency of the RNA produced by the polynucleotide comprising the DNA sequence by identifying the amount of circularized RNA, the amount of excised intronic sequences, the amount of precursor RNA remaining after circularization, and combinations thereof. In some embodiments, the method further comprises comparing the circularization efficiency of the polynucleotide with a polynucleotide comprising a native intronic sequence, or a parent polynucleotide.
   e. Spacer

In various embodiments, a provided polynucleotide (e.g., a DNA template, a precursor RNA polynucleotide, or a circular RNA polynucleotide) comprises one or more spacers.

In certain embodiments, the DNA template, precursor linear RNA polynucleotide and circular RNA provided herein comprise a 5′ and/or a 3′ spacer. In some embodiments, the polynucleotide comprises one or more spacers in the intron elements. In some embodiments, the polynucleotide comprises one or more spacers in the exon elements. In some embodiments, the polynucleotide comprises a spacer in the 3′ intron fragment (also referred to as “5′ external spacer”). In some embodiments, the polynucleotide comprises a spacer in the 5′ intron fragment (also referred to as “3′ external spacer”). In some embodiments, the polynucleotide comprises a spacer in the 3′ exon fragment (also referred to as “5′ internal spacer”). In some embodiments, the polynucleotide comprises a spacer in the 5′ exon fragment (also referred to as “3′ internal spacer”).

In certain embodiments, the polynucleotide comprises a spacer in the 3′ intron fragment and/or a spacer in the 5′ intron fragment. In some embodiments, the 5′ external spacer is located 5′ to the 3′ permuted intron segment. In some embodiments, the 5′ internal spacer is located 3′ to the 3′ exon segment. In some embodiments, the 3′ external spacer is located 3′ to the 5′ permuted intron segment. In some embodiments, the 3′ external spacer is located 5′ to the 5′ exon segment.

In certain embodiments, the polynucleotide comprises a 5′ external spacer located between a leading untranslated sequence and the 5′ or 3′ intron element. In certain embodiments, the polynucleotide comprises a 3′ external spacer located between the 3′ or 5′ intron element and a lagging untranslated sequence.

In certain embodiments where the polynucleotide comprises a monotron, the polynucleotide can comprise an internal spacer sequence positioned between the terminal element and the intervening region, and/or between the intervening region and the monotron element. In certain embodiments, the polynucleotide can comprise an external spacer positioned adjacent to the terminal element and/or an external spacer positioned adjacent to the monotron element.

In certain embodiments, the spacers aid with circularization or protein expression due to symmetry created in the overall sequence of the precursor RNA polynucleotide. In certain embodiments, including a 5′ internal spacer and/or including a spacer between the 3′ group I intron fragment and the intervening region may conserve secondary structures in those regions by preventing them from interacting, thus increasing splicing efficiency. In certain embodiments, there is a spacer, for example, between the 3′ permuted intron segment and the intervening region, wherein the spacer may prevent structured regions of an IRES or aptamer of a TIE comprised in the intervening region from interfering with the folding of the 3′ permuted intron segment or reduces the extent to which this occurs.

In some embodiments, the polynucleotide further comprises an aptamer. In some embodiments, the aptamer is synthetic.

In some embodiments, the first spacer (e.g., between the 3′ group I or II intron fragment and intervening region) and second spacer (e.g., between the two expression sequences and intervening region) comprise additional base pairing regions that are predicted to base pair with each other and not to the first and second duplex regions. In other embodiments, the first spacer (e.g., between 3′ group I or II intron fragment and intervening region) and second spacer (e.g., between the one of the intervening region and 5′ group I or II intron fragment) comprise additional base pairing regions that are predicted to base pair with each other and not to the first and second duplex regions.

In certain embodiments, the polynucleotide comprises a first (5′) and a second (3′) spacer. In some embodiments, the polynucleotide comprises a 5′ external spacer and a 3′ external spacer, wherein the spacers comprise additional base pairing regions that are predicted to base pair with each other and not to the first and second duplex regions. In other embodiments, the polynucleotide comprises a 5′ internal spacer and a 3′ internal spacer, wherein the spacers comprise additional base pairing regions that are predicted to base pair with each other and not to the first and second duplex regions. In some embodiments, such spacer base pairing brings the permuted intron segments in close proximity to each other, which may increase splicing efficiency. Additionally, in some embodiments, the combination of base pairing between the first and second duplex regions, and separately, base pairing between the first and second spacers, promotes the formation of a splicing bubble containing the permuted intron segments flanked by adjacent regions of base pairing.

Typical spacers are contiguous sequences with one or more of the following qualities: (1) predicted to avoid interfering (e.g., forming duplex) with proximal structures, for example, the IRES, expression sequence, aptamer, or intron; (2) is at least 5 nt long and no longer than 100 nt; (3) is located adjacent to the permuted intron segment; and (4) contains one or more of the following: (a) an unstructured region at least 5 nt long, (b) a region of base pairing at least 5 nt long to a distal sequence, such as another spacer, and (c) a structured region at least 5 nt long limited in scope to the sequence of the spacer. In various embodiments, a spacer is not predicted to form a duplex of more than 8 nucleotides in length with any sequences within 250 nucleotides in either direction. In some embodiments, the spacer is not predicted to form a duplex of more than 8 nucleotides in length with any sequences within 1000 nucleotides in either direction. In some embodiments, the spacer comprises an unstructured, structured or randomly generated polynucleotide sequence. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides.

In some embodiments, a spacer sequence is at least 5, 6, 7, 8, 9, 10, 11, 12, 13, 14, 15, 16, 17, 18, 19, 20, 25, or 30 nucleotides in length. In some embodiments, a spacer sequence is no more than 100, 90, 80, 70, 60, 50, 45, 40, 35, or 30 nucleotides in length. In some embodiments, a spacer sequence is between 5 and 50, 10 and 50, 20 and 50, 20 and 40, and/or 25 and 35 nucleotides in length. In certain embodiments, a spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the spacer sequence is at least 5 nucleotides in length, and/or about 5 to about 60 nucleotides in length.

In some embodiments, a spacer sequence is a polyA sequence. In some embodiments, a spacer sequence is a polyAC sequence. In some embodiments, a spacer comprises about 10%, 20%, 30%, 40%, 50%, 60%, 70%, 80%, 90%, or 100% polyAC content. In some embodiments, a spacer comprises about 10%, 20%, 30%, 40%, 50%, 60%, 70%, 80%, 90%, or 100% polypyrimidine (C/T or C/U) content.

f. Duplex

In various embodiments, a provided polynucleotide (e.g., a DNA template, a precursor RNA polynucleotide, or a circular RNA polynucleotide) comprises one or more duplexes.

In some embodiments, the polynucleotide comprises a 5′ external duplex located within the 3′ intron fragment. In some embodiments, the polynucleotide comprises a 3′ external duplex located within the 5′ intron fragment. In some embodiments, the polynucleotide comprises a 5′ internal duplex sequence and a 3′ internal duplex sequence. In some embodiments, the polynucleotide comprises a 5′ internal duplex located within the 3′ exon fragment. In some embodiments, the 5′ internal duplex sequence is positioned between the 5′ exon element and the intervening region. In some embodiments, the polynucleotide comprises a 3′ internal duplex located within the 5′ exon fragment. In some embodiments, the 3′ internal duplex sequence is positioned between the intervening region and the 3′ exon element. In certain embodiments, the polynucleotide comprises a 5′ external duplex located within the 3′ intron fragment and a 3′ external duplex located within the 5′ intron fragment. In some embodiments, the polynucleotide comprises a 5′ internal duplex located within the 3′ exon fragment and a 3′ internal duplex located within the 5′ exon fragment. In some embodiments, the polynucleotide comprises a 5′ external duplex, 5′ internal duplex, a 3′ internal duplex region, and a 3′ external duplex.

In some embodiments, the polynucleotide comprises a monotron element, intervening region, and terminal element, and a 5′ internal duplex sequence and a 3′ internal duplex sequence. In some embodiments, if the terminal element is upstream of the monotron element, the 5′ internal duplex sequence is positioned between the terminal element and the intervening region, and the 3′ internal duplex sequence is positioned between the intervening region and the monotron element. In some embodiments, if the monotron element is upstream of the terminal element, the 5′ internal duplex sequence is positioned between monotron and the intervening region, and the 3′ internal duplex sequence is positioned between the intervening region and the terminal element. In some embodiments, the 5′ or 3′ internal duplex is positioned adjacent to a 5′ or 3′ internal spacer.

In some embodiments, the polynucleotide comprises a first (5′) duplex and a second (3′) duplex (e.g., a 5′ external duplex region and a 3′ external duplex region). In certain embodiments, the first and second duplex regions may form perfect or imperfect duplexes. Thus, in certain embodiments, at least 75%, 80%, 85%, 90%, 91%, 92%, 93%, 94%, 95%, 96%, 97%, 98%, 99% or 100% of the first duplex and second duplex may be base paired with one another. In some embodiments, the duplexes regions are predicted to have less than 50% (e.g., less than 45%, less than 40%, less than 35%, less than 30%, less than 25%) base pairing with unintended sequences in the RNA (e.g., non-duplex sequences). In some embodiments, the 5′ internal duplex sequence and 3′ internal duplex sequence are at least 80%, at least 85%, at least 90%, at least 91%, at least 92%, at least 93%, at least 94%, at least 95%, at least 96%, at least 97%, at least 98%, at least 99% or 100% complementary. In some embodiments, including such first duplex and second duplex on the 5′ and 3′ ends of the precursor RNA strand, respectively, and adjacent or very close to the permuted intron segment, bring the permuted intron segments in close proximity to each other, increasing splicing efficiency.

In some embodiments, a duplex, whether, e.g., a 5′ internal duplex sequence or 3′ internal duplex sequence, is 3-100 nt in length (e.g., 3-75 nt in length, 3-50 nt in length, 20-50 nt in length, 35-50 nt in length, 5-25 nt in length, 5-20 nt in length, 9-19 nt in length). In some embodiments, a duplex has a length of about 9 to about 50 nt. In one embodiment, a duplex has a length of about 9 to about 19 nt. In one embodiment, a duplex has a length of about 5 to about 20 nt nucleotides in length, inclusive. In one embodiment, the 5′ internal duplex sequence and 3′ internal duplex sequence are each independently about 9 to about 50 nt, about 9 to about 19 nt, or about 5 to about 20 nt nucleotides in length, inclusive. In one embodiment, a duplex has a length of about 20 to about 40 nt. In some embodiments, a duplex is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nt in length. In certain embodiments, a duplex has a length of about 30 nt. In certain embodiments, the 5′ and 3′ internal duplex sequences are predicted to form a contiguous duplex. In some embodiments, the contiguous duplex has a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nt in length. In some embodiments, the contiguous duplex has a length of no longer than 35 nucleotides. In some embodiments, at least one of the exon segments is less than 15 nucleotides in length.

In some embodiments, the 5′ internal duplex sequence and/or 3′ internal duplex sequence each have a GC content of at least 10%.

In other embodiments, the polynucleotide does not comprise of any duplex to optimize translation or circularization.

g. Affinity Sequences

In various embodiments, a provided polynucleotide (e.g., a DNA template, a precursor linear RNA polynucleotide, or a circular RNA polynucleotide) may comprise an affinity sequence (or affinity tag) In some embodiments, a precursor RNA polynucleotide comprises at least one affinity tag. In some embodiments, the affinity tag is located in the 3′ intron element. In some embodiments, the affinity tag is located in the 5′ intron element. In some embodiments, both (3′ and 5′) intron elements each comprise an affinity tag. In some embodiments, the 5′ affinity tag is located 5′ to the 3′ permuted intron segment. In some embodiments, the 3′ affinity tag is located 3′ to the 5′ permuted intron segment.

In some embodiments, the polynucleotide comprises a monotron element comprising an affinity tag and/or terminal element comprising an affinity tag. In some embodiments, the terminal element comprises (a) a 5′ affinity tag if the terminal element is located upstream of the monotron element, wherein the 5′ affinity tag is located 5′ to the terminal element; or (b) a 3′ affinity tag if the monotron element is located upstream of the terminal element, wherein the 3′ affinity tag is located 3′ to the terminal element. In some embodiments, the monotron element comprises (a) a 3′ affinity tag if the terminal element is located upstream of the monotron element, wherein the 3′ affinity tag is located 3′ to the monotron element; or (b) a 5′ affinity tag if the monotron element is located upstream of the terminal element, wherein the 5′ affinity tag is located 5′ to the monotron element. In some embodiments, if the precursor RNA polynucleotide comprises an external spacer, the 5′ or 3′ affinity tag is positioned adjacent to the external spacer.

In one embodiment, an affinity tag of the 3′ intron element is the length as an affinity tag in the 5′ intron element. In some embodiments, an affinity tag of the 3′ intron element is the same sequence as an affinity tag in the 5′ intron element. In some embodiments, the affinity sequence is placed to optimize oligo-dT purification.

In some embodiments, the one or more affinity tags present in a precursor RNA polynucleotide are removed upon circularization. See, for example, FIGS. 97A and 97B from WO2022261490, which are incorporated by reference herein in entirety. In some embodiments, affinity tags are added to remaining linear RNA after circularization of precursor RNA is performed. In some such embodiments, affinity tags are added enzymatically to linear RNA. The presence of one or more affinity tags in linear RNA and their absence from circular RNA can facilitate purification of circular RNA. In some embodiments, such purification is performed using a negative selection or affinity-purification method. In some embodiments, such purification is performed using a binding agent that preferentially or specifically binds to the affinity tag.

In some embodiments, an affinity sequence, such as biotin, is added to linear RNA by ligation. In some embodiments, an oligonucleotide comprising an affinity sequence is ligated to linear RNA. In some embodiments, an oligonucleotide conjugated to an affinity handle is ligated to the linear RNA. In some embodiments, a solution comprising the linear RNA ligated to the affinity sequence or handle and the circular RNA that does not comprise an affinity sequence or handle are contacted with a binding agent comprising a solid support conjugated to an oligonucleotide complementary to the affinity sequence or to a binding partner of the affinity handle, such that the linear RNA binds to the binding agent, and the circular RNA is eluted or separated from the solid support.

In some embodiments, an affinity tag comprises a polyA sequence or is a polyA affinity tag. In some embodiments the polyA sequence is at least 15, 30, or 60 nt in length. In some embodiments, the affinity tag comprising a polyA sequence is present in two places in a precursor linear RNA. In some embodiments, one or both polyA sequences are 15-50 nt in length. In some embodiments, one or both polyA sequences are 20-25 nt in length. In some embodiments, the polyA sequence(s) is removed upon circularization. Thus, an oligonucleotide hybridizing with the polyA sequence, such as a deoxythymidine oligonucleotide (oligo(dT)) conjugated to a solid surface (e.g., a resin), can be used to separate circular RNA from its precursor RNA.

Any purification method for circular RNA described herein may comprise one or more buffer exchange steps. In some embodiments, buffer exchange is performed after in vitro transcription (IVT) and before additional purification steps. In some such embodiments, the IVT reaction solution is buffer exchanged into a buffer comprising Tris. In some embodiments, the IVT reaction solution is buffer exchanged into a buffer comprising greater than 1 mM or greater than 10 mM one or more monovalent salts, such as NaCl or KCl, and optionally comprising EDTA. In some embodiments, buffer exchange is performed after purification of circular RNA is complete. In some embodiments, buffer exchange is performed after IVT and after purification of circular RNA. In some embodiments, the buffer exchange that is performed after purification of circular RNA comprises exchange of the circular RNA into water or storage buffer. In some embodiments, the storage buffer comprises 1 mM sodium citrate, pH 6.5.

h. Leading Sequences & Lagging Sequences

In various embodiments, provided polynucleotide (e.g., a DNA template, a precursor linear RNA polynucleotide, a linear mRNA polynucleotide, or a circular RNA polynucleotide) comprises a leading untranslated sequence. In some embodiments, the leading untranslated sequence is located at the 5′ end in the 3′ intron fragment (also referred to as “5′ leading sequence”). In some embodiments, the leading untranslated sequence comprises the last nucleotide of a transcription start site (TSS). In some embodiments, the TSS is chosen from a viral, bacterial, or eukaryotic DNA template. In one embodiment, the leading untranslated sequence comprises the last nucleotide of a TSS and 0 to 100 additional nucleotides. In some embodiments, the TSS is a spacer. In some embodiments, the leading untranslated sequence contains a guanosine at the 5′ end.

In various embodiments, provided polynucleotide (e.g., a DNA template, a precursor linear RNA polynucleotide, a linear mRNA polynucleotide, or a circular RNA polynucleotide) comprises a lagging untranslated sequence (also referred to as “trailing sequence”). In some embodiments, the lagging untranslated sequence is located at the 3′ end. In some embodiments, the polynucleotide comprises a 3′ external spacer located between the 3′ intron element and a lagging untranslated sequence. In some embodiments, the polynucleotide a leading untranslated sequence at the 5′ end. In some embodiments, the polynucleotide comprises a 5′ external spacer located between a leading untranslated sequence and the 5′ intron element.

In some embodiments, the polynucleotide comprises a monotron element and a leading untranslated sequence. In some embodiments, the polynucleotide comprises a 5′ external spacer positioned between a leading untranslated sequence and either the terminal element or monotron element. In some embodiments, the polynucleotide comprises a monotron element and a lagging untranslated sequence. In some embodiments, the polynucleotide comprises a 3′ external spacer positioned between the lagging untranslated sequence and either the monotron element or terminal element.

In some embodiments, the lagging untranslated sequence comprises a restriction site sequence or a fragment thereof. In certain embodiments, the restriction site sequence or fragment thereof is used to linearize the polypeptide (e.g., DNA template). In some embodiments, the restriction site sequence is derived from a natural viral, bacterial or eukaryotic DNA template.

### C. Intervening Region

In various embodiments, a provided polynucleotide (e.g., a DNA template, a linear precursor RNA polynucleotide, a linear mRNA polynucleotide, or a circular RNA polynucleotide) comprises an intervening region.

In some embodiments, the intervening region and/or core functional element comprises one or more noncoding elements, e.g., microRNA binding site, IRES transacting factor region, restriction site, an RNA editing region, structural or sequence element, a granule site, a zip code element, or an RNA trafficking element. In some embodiments, the intervening region and/or core functional element comprises one or more expression sequences encoding a CD19 binding molecule, e.g., CD19 CAR. In some embodiments, the intervening region and/or core functional element comprises a combination of coding and noncoding elements. In some embodiments, the coding or non-coding region is a part of the core functional element or intervening region located between the 5′ end and 3′ end of the linear precursor RNA polynucleotide and resultant circular RNA.

In some embodiments, the coding element encodes two or more polypeptides (e.g., wherein at least one polypeptide comprises a CD19 binding molecule). In some embodiments, the coding element may encode one or more subunits of a polynucleotide. In some embodiments, the sequences encoding the two or more polypeptides are separated by a ribosomal skipping element or a nucleotide sequence encoding a protease cleavage site. In certain embodiments, the ribosomal skipping element encodes thosea-asigna virus 2A peptide (T2A), porcine teschovirus-1 2 A peptide (P2A), foot-and-mouth disease virus 2 A peptide (F2A), equine rhinitis A vims 2A peptide (E2A), cytoplasmic polyhedrosis vims 2A peptide (BmCPV 2A), or flacherie vims of B. mori 2A peptide (BmIFV 2A). Coding elements or regions and payloads are described in further detail elsewhere herein.

In some embodiments, the intervening region comprises at least one translation initiation element (TIE). TIEs are designed to allow translation efficiency of an encoded protein. In some embodiments, core functional elements comprising one or more coding elements will further comprise one or more TIEs. In some embodiments, a translation initiation element (TIE) comprises a synthetic TIE. In some embodiments, a synthetic TIE comprises aptamer complexes, synthetic IRES or other engineered TIEs capable of initiating translation of a linear RNA or circular RNA polynucleotide. In certain embodiments, the presence of a synthetic TIE (e.g., a synthetic IRES) in a circular RNA polynucleotide may enhance translation compared to circular RNA polynucleotide comprising a non-synthetic TIE (e.g., a non-synthetic IRES). In certain embodiments, the presence of a synthetic TIE (e.g., a synthetic IRES) in a circular RNA polynucleotide may have comparable translation functions as compared to a circular RNA polynucleotide comprising a non-synthetic TIE (e.g., non-synthetic IRES).

In some embodiments, the intervening region comprises one or more noncoding elements. In some embodiments, the noncoding element comprises an untranslated region (UTR) or fragment thereof. In some embodiments, the noncoding element is a natural 5′ UTR. In some embodiments, the noncoding element is a natural 3′ UTR. In some embodiments, the noncoding element is a synthetic spacer sequence. In some embodiments, the noncoding element is an aptamer. In some embodiments, the noncoding element is or comprises a translation initiation element (TIE). In some embodiments, the noncoding element comprises a lncRNA, miRNA, or a miRNA sponge.

In some embodiments, the intervening region comprises a TIE comprising an untranslated region (UTR) or a fragment thereof, an aptamer complex or a fragment thereof, or a combination thereof. In certain embodiments, the TIE contains modified nucleotides.

In certain embodiments, the TIE provided herein comprise an internal ribosome entry site (IRES). In certain embodiments, the TIE provided herein comprise a viral or eukaryotic internal ribosome entry site (IRES) or a fragment or variant thereof. In certain embodiments, the IRES comprises one or more modified nucleotides compared to the wild-type viral IRES or eukaryotic IRES. See, e.g., PCT Application No. US2022/33091, which is incorporated herein by reference in its entirety.

In some embodiments, the noncoding element comprises an untranslated region (UTR). In some embodiments, the noncoding element is a natural 5′ UTR. In some embodiments, the noncoding element is a natural 3′ UTR. In some embodiments, the noncoding element is a synthetic spacer sequence. In some embodiments, the noncoding element is an aptamer or synthetic aptamer. In some embodiments, the noncoding element is or comprises a translation initiation element (TIE).

a. Translation Initiation Element

In some embodiments, the DNA template, linear RNA polynucleotide, and circular RNA polynucleotide comprise an intervening region and/or core functional element. In some embodiments, the intervening region and/or core functional element comprises an expression sequence encoding a CD19 binding molecule, e.g., CD19 CAR. In some embodiments, the intervening region and/or core functional element further comprises a translation initiation element (TIE) upstream to the expression sequence encoding the CD19 binding molecule, and/or a termination element.

In some embodiments, the polynucleotide comprises a translation initiation element (TIE). In some embodiments, the intervening region comprises at least one TIE. In some embodiments, the TIE is upstream to the expression sequence encoding the CD19 binding molecule. In some embodiments, TIEs are designed to allow translation efficiency of an encoded protein (e.g., a CD19 binding molecule). Accordingly, in some embodiments, an intervening region comprising one or more coding elements further comprises one or more TIEs. In other embodiments, an intervening region comprising only noncoding elements lacks any TIEs.

In some embodiments, a TIE comprises an internal ribosome entry site (IRES). In certain embodiments, the TIE provided herein comprise a viral or eukaryotic internal ribosome entry site (IRES) or a fragment or variant thereof. In some embodiments, inclusion of an IRES permits the translation of one or more open reading frames from a circular RNA (e.g., open reading frames that form the expression sequences). In some embodiments, IRES attracts a eukaryotic ribosomal translation initiation complex and promotes translation initiation. See, e.g., PCT Publication No. WO202261490, which is incorporated herein by reference in its entirety.

i. Natural TIES: Viral & Eukaryotic/Cellular IRES

In certain embodiments, as provided herein, the payload encoded by the circular RNA polynucleotide may be optimized through use of a specific internal ribosome entry sites (IRES) within the translation initiation element (TIE). In some embodiments, IRES specificity within a circular RNA can significantly enhance expression of specific proteins encoded within the coding element. In some embodiments, the IRES comprises a viral IRES or eukaryotic IRES.

A multitude of IRES sequences are available and include sequences derived from a wide variety of viruses, such as from leader sequences of picornaviruses such as the encephalomyocarditis virus (EMCV) UTR (Jang et al., J. Virol. (1989) 63: 1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161. In some embodiments, the IRES is capable of facilitating expression of a protein encoded by the precursor RNA in a cell. In some embodiments, the IRES is capable of facilitating expression of the protein, such that the expression level of the protein is comparable to or higher than when a control IRES is used.

Different IRES sequences have varying ability to drive protein expression, and the ability of any particular identified or predicted IRES sequence to drive protein expression from linear mRNA or circular RNA constructs is unknown and unpredictable. In certain embodiments, potential IRES sequences can be bioinformatically identified based on sequence positions in viral sequences. However, the activity of such sequences has been previously uncharacterized. As demonstrated herein, such IRES sequences may have differing protein expression capability depending on cell type, for example in T cells, liver cells, or muscle cells. In some embodiments, the novel IRES sequences described herein may have at least 2, 3, 4, 5, 6, 7, 8, 9, 10, 20, 50, or 100 fold increased expression in a particular cell type compared to previously described EMCV IRES sequences.

In some embodiments, for driving protein expression, a polynucleotide (e.g., a DNA template, a linear precursor RNA polynucleotide, or a circular RNA polynucleotide) comprises an IRES operably linked to a protein coding sequence. In some embodiments, the IRES comprises a sequence selected from the sequences in Table 4 or a fragment thereof or a sequence from PCT/US2022/033091 or PCT/US2023/084046. In some embodiments, the IRES comprises a sequence at least 80%, 85%, 90%, 91%, 92%, 93%, 94%, 95%, 96%, 97%, 98%, or 99% identical to a sequence selected from the sequences in Table 4 or a fragment thereof or a sequence from PCT/US2022/033091 or PCT/US2023/084046.

In some embodiments, the IRES is derived from Aalivirus, Ailurivirus, Ampivirus, Anativirus, Aphthovirus, Aquamavirus, Avihepatovirus, Avisivirus, Boosepivirus, Bopivirus, Caecilivirus, Cardiovirus, Cosavirus, Crahelivirus, Crohivirus, Danipivirus, Dicipivirus, Diresapivirus, Enterovirus, Erbovirus, Felipivirus, Fipivirus, Gallivirus, Gruhelivirus, Grusopivirus, Harkavirus, Hemipivirus, Hepatovirus, Hunnivirus, Kobuvirus, Kunsagivirus, Limnipivirus, Livupivirus, Ludopivirus, Malagasivirus, Marsupivirus, Megrivirus, Mischivirus, Mosavirus, Mupivirus, Myrropivirus, Orivirus, Oscivirus, Parabovirus, Parechovirus, Pasivirus, Passerivirus, Pemapivirus, Poecivirus, Potamipivirus, Pygoscepivirus, Rabovirus, Rafivirus, Rajidapivirus, Rohelivirus, Rosavirus, Sakobuvirus, Salivirus, Sapelovirus, Senecavirus, Shanbavirus, Sicinivirus, Symapivirus, Teschovirus, Torchivirus, Tottorivirus, Tremovirus, Tropivirus, Hepacivirus, Pegivirus, Pestivirus, or Flavivirus. In some embodiments herein, the IRES is selected from an Enterovirus, Kobuvirus, Parechovirus, Hunnivirus, Passerivirus, Mischivirus, and Cardiovirus.

In some embodiments, the IRES is an IRES sequence derived from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAPl, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24, or an aptamer to eIF4G.

In some embodiments, the IRES comprises in whole or in part a eukaryotic or cellular IRES. In certain embodiments, the IRES is an IRES sequence derived from a human gene, wherein the human gene is ABCF1, ABCG1, ACAD10, ACOT7, ACSS3, ACTG2, ADCYAP1, ADK, AGTR1, AHCYL2, AHI1, AKAP8L, AKR1A1, ALDH3A1, ALDOA, ALG13, AMMECR1L, ANGPTL4, ANK3, AOC3, AP4B1, AP4E1, APAF1, APBB1, APC, APH1A, APOBEC3D, APOM, APP, AQP4, ARHGAP36, ARL13B, ARMC8, ARMCX6, ARPC1A, ARPC2, ARRDC3, ASAP1, ASB3, ASB5, ASCL1, ASMTL, ATF2, ATF3, ATG4A, ATP5B, ATP6VOA1, ATXN3, AURKA, AURKA, AURKA, AURKA, B3GALNT1, B3GNTL1, B4GALT3, BAAT, BAG1, BAIAP2, BAIAP2L2, BAZ2A, BBX, BCAR1, BCL2, BCS1L, BET1, BID, BIRC2, BPGM, BPIFA2, BRINP2, BSG, BTN3A2, C12orf43, C14orf93, C17orf62, C1orf226, C21orf62, C2orf15, C4BPB, C4orf22, C9orf84, CACNA1A, CALCOCO2, CAPN11, CASP12, CASP8AP2, CAV1, CBX5, CCDCl20, CCDCl7, CCDCl86, CCDC51, CCN1, CCND1, CCNT1, CD2BP2, CD9, CDC25C, CDC42, CDC7, CDCA7L, CDIP1, CDK1, CDK11A, CDKN1B, CEACAM7, CEP295NL, CFLAR, CHCHD7, CHIA, CHIC1, CHMP2A, CHRNA2, CLCN3, CLEC12A, CLEC7A, CLECL1, CLRN1, CMSS1, CNIH1, CNR1, CNTN5, COG4, COMMD1, COMMD5, CPEB1, CPS1, CRACR2B, CRBN, CREM, CRYBG1, CSDE1, CSF2RA, CSNK2A1, CSTF3, CTCFL, CTH, CTNNA3, CTNNB1, CTNNB1, CTNND1, CTSL, CUTA, CXCR5, CYB5R3, CYP24A1, CYP3A5, DAG1, DAP3, DAP5, DAXX, DCAF4, DCAF7, DCLRElA, DCP1A, DCTN1, DCTN2, DDX19B, DDX46, DEFB123, DGKA, DGKD, DHRS4, DHX15, DI03, DLG1, DLL4, DMD UTR, DMD ex5, DMKN, DNAH6, DNAL4, DUSP13, DUSP19, DYNC1I2, DYNLRB2, DYRKIA, ECI2, ECT2, EIF1AD, EIF2B4, EIF4G1, EIF4G2, EIF4G3, ELANE, ELOVL6, ELP5, EMCN, ENO1, EPB41, ERMN, ERVV-1, ESRRG, ETFB, ETFBKMT, ETV1, ETV4, EXD1, EXT1, EZH2, FAM111B, FAM157A, FAM213A, FBXO25, FBXO9, FBXW7, FCMR, FGF1, FGF1, FGF1A, FGF2, FGF2, FGF-9, FHL5, FMR1, FN1, FOXP1, FTH1, FUBP1, G3BP1, GABBR1, GALC, GART, GAS7, gastrin, GATA1, GATA4, GFM2, GHR, GJB2, GLI1, GLRA2, GMNN, GPAT3, GPATCH3, GPR137, GPR34, GPR55, GPR89A, GPRASP1, GRAP2, GSDMB, GSTO2, GTF2B, GTF2H4, GUCYlB2, HAX1, HCST, HIGD1A, HIGD1B, HIPK1, HISTIHIC, HIST1H3H, HK1, HLA-DRB4, HMBS, HMGA1, HNRNPC, HOPX, HOXA2, HOXA3, HPCAL1, HR, HSP90AB1, HSPA1A, HSPA4L, HSPA5, HYPK, IFFO1, IFT74, IFT81, IGF1, IGF1R, IGF1R, IGF2, IL11, IL17RE, ILIRL1, IL1RN, IL32, IL6, ILF2, ILVBL, INSR, INTS13, IP6K1, ITGA4, ITGAE, KCNE4, KERA, KIAA0355, KIAA0895L, KIAA1324, KIAA1522, KIAA1683, KIF2C, KIZ, KLHL31, KLK7, KRR1, KRT14, KRT17, KRT33A, KRT6A, KRTAP10-2, KRTAP13-3, KRTAP13-4, KRTAP5-11, KRTCAP2, LACRT, LAMB1, LAMB3, LANCL1, LBX2, LCAT, LDHA, LDHAL6A, LEF1, LINC-PINT, LMO3, LRRC4C, LRRC7, LRTOMT, LSM5, LTB4R, LYRM1, LYRM2, MAGEA11, MAGEA8, MAGEB1, MAGEB16, MAGEB3, MAPT, MARS, MC1R, MCCC1, METTL12, METTL7A, MGC16025, MGC16025, MIA2, MIA2, MITF, MKLN1, MNT, MORF4L2, MPD6, MRFAP1, MRPL21, MRPS12, MSI2, MSLN, MSN, MT2A, MTFR1L, MTMR2, MTRR, MTUS1, MYB, MYC, MYCL, MYCN, MYL10, MYL3, MYLK, MYO1A, MYT2, MZB1, NAPIL1, NAV1, NBAS, NCF2, NDRG1, NDST2, NDUFA7, NDUFB11, NDUFC1, NDUFS1, NEDD4L, NFAT5, NFE2L2, NFE2L2, NFIA, NHEJ1, NHP2, NIT1, NKRF, NME1-NME2, NPAT, NR3C1, NRBF2, NRF1, NTRK2, NUDCD1, NXF2, NXT2, ODC1, ODF2, OPTN, OR10R2, OR11L1, OR2M2, OR2M3, OR2M5, OR2T10, OR4C15, OR4F17, OR4F5, OR5H1, OR5K1, OR6C3, OR6C75, OR6N1, OR7G2, p53, P2RY4, PAN2, PAQR6, PARP4, PARP9, PC, PCBP4, PCDHGC3, PCLAF, PDGFB, PDZRN4, PELO, PEMT, PEX2, PFKM, PGBD4, PGLYRP3, PHLDA2, PHTF1, PI4 KB, PIGC, PIM1, PKD2L1, PKM, PLCB4, PLD3, PLEKHA1, PLEKHB1, PLS3, PML, PNMA5, PNN, POC1A, POC1B, POLD2, POLD4, POU5F1, PPIG, PQBP1, PRAME, PRPF4, PRR11, PRRT1, PRSS8, PSMA2, PSMA3, PSMA4, PSMD11, PSMD4, PSMD6, PSME3, PSMG3, PTBP3, PTCH1, PTHLH, PTPRD, PUS7L, PVRIG, QPRT, RAB27A, RAB7B, RABGGTB, RAET1E, RALGDS, RALYL, RARB, RCVRN, REG3G, RFC5, RGL4, RGS19, RGS3, RHD, RINL, RIPOR2, RITA1, RMDN2, RNASE1, RNASE4, RNF4, RPA2, RPL17, RPL21, RPL26L1, RPL28, RPL29, RPL41, RPL9, RPS11, RPS13, RPS14, RRBP1, RSU1, RTP2, RUNX1, RUNX1T1, RUNX1T1, RUNX2, RUSC1, RXRG, S100A13, S100A4, SAT1, SCHIP1, SCMH1, SEC14L1, SEMA4A, SERPINA1, SERPINB4, SERTAD3, SFTPD, SH3D19, SHC1, SHMT1, SHPRH, SIM1, SIRT5, SLC11A2, SLC12A4, SLC16A1, SLC25A3, SLC26A9, SLC5A11, SLC6A12, SLC6A19, SLC7A1, SLFN11, SLIRP, SMAD5, SMARCADI, SMN1, SNCA, SNRNP200, SNRPB2, SNX12, SOD1, SOX13, SOX5, SP8, SPARCL1, SPATA12, SPATA31C2, SPN, SPOP, SQSTM1, SRBD1, SRC, SREBF1, SRPK2, SSB, SSB, SSBP1, ST3GAL6, STAB1, STAMBP, STAU1, STAU1, STAU1, STAU1, STAU1, STK16, STK24, STK38, STMN1, STX7, SULT2B1, SYK, SYNPR, TAF1C, TAGLN, TANK, TAS2R40, TBC1D15, TBXAS1, TCF4, TDGF1, TDP2, TDRD3, TDRD5, TESK2, THAP6, THBD, THTPA, TIAM2, TKFC, TKTL1, TLR10, TM9SF2, TMC6, TMCO2, TMED10, TMEM116, TMEM126A, TMEM159, TMEM208, TMEM230, TMEM67, TMPRSS13, TMUB2, TNFSF4, TNIP3, TP53, TP53, TP73, TRAF1, TRAK1, TRIM31, TRIM6, TRMT1, TRMT2B, TRPM7, TRPM8, TSPEAR, TTC39B, TTLL11, TUBB6, TXLNB, TXNIP, TXNL1, TXNRD1, TYROBP, U2AF1, UBA1, UBE2D3, UBE2I, UBE2L3, UBE2V1, UBE2V2, UMPS, UNG, UPP2, USMG5, USP18, UTP14A, UTRN, UTS2, VDR, VEGFA, VEGFA, VEPH1, VIPAS39, VPS29, VSIG10L, WDHD1, WDR12, WDR4, WDR45, WDYHV1, WRAP53, XIAP, XPNPEP3, YAP1, YWHAZ, YY1AP1, ZBTB32, ZNF146, ZNF250, ZNF385A, ZNF408, ZNF410, ZNF423, ZNF43, ZNF502, ZNF512, ZNF513, ZNF580, ZNF609, ZNF707, or ZNRD1.

In some embodiments, the cell is a myotube. In some embodiments, the IRES is derived from Bopivirus, Oscivirus, Hunnivirus, Passerivirus, Mischivirus, Kobuvirus, Enterovirus, Cardiovirus, Salivirus, Rabovirus, Parechovirus, Gallivirus, or Sicinivirus. In some embodiments, the IRES is derived from Hunnivirus, Passerivirus, Kobuvirus, Bopivirus, or Enterovirus. In some embodiments, the IRES is derived from Enterovirus I, Enterovirus F, Enterovirus E, Enterovirus J, Enterovirus C, Enterovirus A, Enterovirus B, Aichivirus B, Parechovirus A, Cardiovirus F, Cardiovirus B, or Cardiovirus E.

In some embodiments, the cell is a hepatocyte. In some embodiments, the IRES is derived from Enterovirus, Bopivirus, Mischivirus, Gallivirus, Oscivirus, Cardiovirus, Kobuvirus, Rabovirus, Salivirus, Parechovirus, Hunnivirus, Tottorivirus, Passerivirus, Cosavirus, or Sicinivirus. In some embodiments, the IRES is derived from Enterovirus, Mischivirus, Kobuvirus, Bopivirus, or Gallivirus. In some embodiments, the IRES is derived from Enterovirus B, Enterovirus A, Enterovirus D, Enterovirus J, Enterovirus C, Rhinovirus B, Enterovirus H, Enterovirus I, Enterovirus E, Enterovirus F, Aichivirus B, Aichivirus A, Parechovirus A, Cardiovirus F, Cardiovirus E, or Cardiovirus B.

In some embodiments, the cell is a T cell. In some embodiments, the IRES is derived from Passerivirus, Bopivirus, Hunnivirus, Mischivirus, Enterovirus, Kobuvirus, Rabovirus, Tottorivirus, Salivirus, Cardiovirus, Parechovirus, Megrivirus, Allexivirus, Oscivirus, or Shanbavirus. In some embodiments, the IRES is derived from Passerivirus, Hunnivirus, Mischivirus, Enterovirus, or Kobuvirus. In some embodiments, the IRES is derived from Enterovirus I, Enterovirus D, Enterovirus C, Enterovirus A, Enterovirus J, Enterovirus H, Aichivirus B, Parechovirus A, or Cardiovirus B.

In some embodiments, for driving protein expression, a provided circular RNA comprises an IRES operably linked to a protein coding sequence. In some embodiments, the IRES comprises a sequence selected from Table 4, or SEQ ID NOs: 1-2983 and 3282-3287 of PCT/US2022/33091 or a fragment thereof, or a sequence from PCT/US2023/084046. In some embodiments, the IRES comprises a sequence at least 80%, 85%, 90%, 91%, 92%, 93%, 94%, 95%, 96%, 97%, 98%, or 99% identical to a sequence selected from Table 4, or a sequence selected from SEQ ID NOs: 1-2983 and 3282-3287 of PCT/US2022/33091, or a sequence from PCT/US2023/084046. In some embodiments, the circular RNA disclosed herein comprises an IRES sequence at least 80%, 85%, 90%, 91%, 92%, 93%, 94%, 95%, 96%, 97%, 98%, or 99% identical to a sequence selected from Table 4, or SEQ ID NOs: 1-2983 and 3282-3287 of PCT/US2022/33091 or a fragment thereof, or a sequence from PCT/US2023/084046. In some embodiments, the circular RNA disclosed herein comprises an IRES sequence selected from Table 4, or SEQ ID NOs: 1-2983 and 3282-3287 of PCT/US2022/33091 or a fragment thereof, or a sequence from PCT/US2023/084046.

Further exemplary IRES sequences are provided in Table 4. In some embodiments, the precursor RNA polynucleotide, circular RNA constructs and related pharmaceutical compositions disclosed herein comprise an IRES sequence that is at least 80%, 85%, 90%, 91%, 92%, 93%, 94%, 95%, 96%, 97%, 98%, or 99% identical to an IRES sequence in Table 4. In some embodiments, the precursor RNA polynucleotide, circular RNA constructs and related pharmaceutical compositions disclosed herein comprise an IRES sequence in Table 4.

TABLE 4
IRES Sequences

| ID | Sequence |
|---|---|
| 4-1 | TTAAAACAGCTCTGGGGTTGTTCCCACCCCAGAGGCCCACGTGGCGGCCAG |
| | TACTCCGGTATTACGGTACCCTTGTACGCCTGTTTTATACTCCCTTCCCCTGT |
| | AACTTAGAAGCATACAAACCAAGTTCAATAGAAGGGGGTACAAACCAGTA |
| | CCACCACGAACAAGCACTCCTGTTTCCCCGGTGACATTGCATAGACTGTAC |
| | CCACGGTTGAAAGCGATCGATCCGTTACCCGCTCCTGTACTTCGAGAAGCC |
| | TAGTATCATCTTGGAATCTTCGATGCGTTGCGCTCAGCACTCAACCCCAGAG |
| | TGTAGCTTAGGCTGATGAGTCTGGACGTCCCCCACCGGCGACGGTGGTCCA |
| | GGCTGCGTTGGCGGCCTACCTGTGGCCCAAAGCCACAGGACGCTAGTTGTG |
| | AACAAGGTGTGAAGAGCCTATTGAGCTACAAGAGAGTCCTCCGGCCCCTGA |
| | ATGCGGCTAATCCTAACCACGGAGCAGGCAGTTGCAAACCAGCAACCGGC |
| | CTGTCGTAACGCGCAAGTCTGTGGCGGAACCGACTACTTTGGGTGTCCGTG |
| | TTTCCTTTTATTTTTACAATGGCTGCTTATGGTGACAATCATAGATTGTTATC |
| | ATAAAGCGACTTGGATTGGCCATCCGGTGAAAGTAAAACACATTGTTTACT |
| | TGTTTGTTGGATTCACTCCAATTAACACTTTTACTTACAAACTCATTACAAC |
| | AACTCTATTAATTAGAGATAAGCATCACA (SEQ ID NO: 200) |
| 4-2 | TTTCCCCTGTTCGTAACTAAGTGTGTGCCCAATCTCCTCACTCCTGCTGGCTT |
| | CACCGACCGGCAGTGTCCAAAATGCTAGGTGAATCCCCTCCCTTTCCTCTGG |
| | GCTTCTGCCCAGCTTCCTCCCCCCAGCCTGACGTGACACAGGCTGTGCAAA |
| | GACCCCGCGAAAGCTGCCAAAAGTGGCAATTGTGGGTCCCCCCTTTGTAAA |
| | GGCGTCGAGTCTTTCTCCCTCAAGGCTAGACCCGTCAGTGAATTCTGTCGGG |
| | CAACTAGTGACGCCACTGCACGCCTCTGACCTCGGCCGCGGAGTGCTGCCC |
| | CCCAAGTCGTGCCCCTGACCACAAGTTGTGCTGTCTGGCAAACATTGTCTGT |
| | GAGAATGTTCCGCTGTGGCTGCCAAGCCTGGCAACAGGCTGCCCCAGTGTG |
| | CGTAGTTCTCATCCAGACTTCGGTCTGGCAACTTGCTGTTAAGACACGGCGT |
| | AAGGGGCGTGTGCCAACGCCCTGGAACGAGTGTCCACTCTAATACCCCGAG |
| | GAATGCTACGCAGGTACCCCTGGTTCGCCAGGGATCTGAGCGTAGGCTAAT |
| | TGTCTAAGGGTATTTTCATTTCCCATTCTTTCTTTCTTGTTCATA (SEQ ID NO: |
| | 201) |
| 4-3 | TCCCCGGCATGAGAGGAATAGACTCTTTCAGGGTTGAAGCCACGAGTGTCG |
| | TTACCCGCACTGGTACTACGCAAAGCCTAGTAACATCTTGAAACTCTTTTTG |
| | GTTGGTCGTTCCACTAGTTACCCCCTAGTAGACCTGGCAGATGAGGCAGGA |
| | CGCTCCCCACTGGCGACAGTGGTCCTGCCTGCGTGGCTGCCTGCACACCCTT |
| | CGGGGTGTGAAGCCAAAAGAAAGACAAGGTGTGAAGAGCCCCGTGTGCTA |
| | CCAGTGAATCCTCCGGCCCCTGAATGCGGCTAATCTTACCCCACAGCTATTG |
| | CACACAATCCAGTGTGTATGTAGTCGTAATGAGCAATTGTGGGACGGAACC |
| | GACTACTTTGGGTGTCCGTGTTTCCTTTTATTCCCATGTTTCTGCTTATGGTG |
| | ACAATACTGACGTATAGTGTTGTTACC (SEQ ID NO: 202) |
| 4-4 | TTAAAACAGCGGATGGGTATCCCACCATCCGGCCCACTGGGTGTAGTACTC |
| | TGGTACATTGTACCTTTGTACGCCTGTTTTCCCCCTCTTGTACCCGCCCTTCA |
| | AGCTCCTTGCCCAAGTAACGTTAGAAGTTTGAACATTGGTACAATAGGAAG |
| | CATCACATCCAGTGGTGTACTGTACAAACACTTCTGTTGCCCCGGAGCGAG |
| | GTATAGATGGTCCCCACCGTCAAAAGCCTTTAACCGTTATCCGCCAATCAA |
| | CTACGTAATGGCTAGTAGCACCTTGGATTTAAGTTGGCGTTCGATCAGGTG |
| | GTAACCCCCACTAGTTTGGTCGATGAGGCTAGGAATTCCCCACGGGTGACC |
| | GTGTCCTAGCCTGCGTGGCGGCCAACCCAGCATCCGCTGGGACGCCAATTT |
| | AATGACATGGTGTGAAGACCTGCATGTGCTTGATTGTGAGTCCTCCGGCCC |
| | CTGAATGCGGCTAACCCTAACCCCGGAGCCTTGCAGCACAATCCAGTGTTG |
| | TTAAGGTCGTAATGAGCAATTCTGGGATGGGACCGACTACTTTGGGTGTCC |
| | GTGTTTCTTATTTTTCTTGAATTTTTCTTATGGTCACAGCATATATACATTAT |
| | ATACTGTGATC (SEQ ID NO: 203) |
| 4-5 | TTAAAATAGCCTCAGGGTTGTTCCCACCCTGAGGGCCCACGTGGTGTAGTA |
| | CTCTGGTATTACGGTACCTTTGTACGCCTATTTTATACCCCCTTCCCCAAGT |
| | AATTTAGAAGCAAGCACAAACCAGTTCAGTAGTAAGCAGTACAATCCAGTA |
| | CTGTAATGAACAAGTACTTCTGTTACCCCGGAAGGGTCTATCGGTAAGCTG |
| | TACCCACGGCTGAAGAATGACCTACCGTTAACCGGCTACCTACTTCGAGAA |
| | GCCTAGTAATGCCGTTGAAGTTTTATTGACGTTACGCTCAGCACACTACCCC |
| | GTGTGTAGTTTTGGCTGATGAGTCACGGCACTCCCCACGGGCGACCGTGGC |
| | CGTGGCTGCGTTGGCGGCCAACCAAGGAGTGCAAGCTCCTTGGACGTCATA |
| | TTACAGACATGGTGTGAAGAGCCTATTGAGCTAGGTGGTAGTCCTCCGGCC |
| | CCTGAATGCGGCTAATCCTAACTCCGGAGCATATCGGTGCGAACCAGCACT |
| | TGGTGTGTTGTAATACGTAAGTCTGGAGCGGAACCGACTACTTTGGGTGTC |
| | CGTGTTTCCTGTTTTAACTTTTATGGCTGCTTATGGTGACAATTTAACATTGT |
| | TACCATATAGCTGTTGGGTTGGCCATCCGGATTTTGTTATAAAACCATTTCC |
| | TCGTGCCTTGACCTTTAACACATTTGTGAACTTCTTTAAATCCCTTTTATTAG |
| | TCCTTAAATACTAAGA (SEQ ID NO: 204) |
| 4-6 | TTCAAACAGCCTGGGGGTTGTACCCACCCCTGGGGCCCACGTGGCGCTAGT |
| | ACTCTGGTACGTTAGTACCTTTGTACGCCTGTTTTCCCCTCCCTTAAACAAA |
| | TTAAGATTACCACTACTGAGGGGAGTAGTCCGACTCCGCTCCGGTACTGCC |
| | GCACCAGTACTCCGGTACACTTAGTACCCTAGTACGGAGTAGATGGTATCC |
| | CCACCCCGCAACTTAGAAGCATGCAAACAAACCGACCAATAGGCGCACGA |
| | TATCCAGTCGTGTTTCGGTCAAGCACTTCTGTCTCCCCGGTCCGAAAGGATC |
| | GTTACCCGCCCGACCCACTACGAGAAGCCCAGTAACTGGCCAAGTGATTGC |
| | GAAGTTGCGCTCAGCCACAACCCCAGTGGTAGCTCTGGAAGATGGGGCTCG |
| | CGTCTCCCCCGTGGTGACACGGTCGCTTGCCCGCGTGTGCTTCCGGGTTCGG |
| | CCTACGCCGTTCACTTCAATGTCACGTAACCAGCCAAGAGCCTATTGTGCTG |
| | GGACGGTTTTCCTCCGGGGCCGTGAATGCTGCTAATCCCAACCTCCGAGCG |
| | TGTGCGCACAACCCAGTGTTGCTACGTCGTAATGCGTAAGTTGGAGGCGGA |
| | ACAGACTACTTTCGGTACCCCGTGTTTCCTTTAAATTTTATTCATTATTTTAT |
| | GGTGACAATTGCTGAGATCTGCGAATTAGCGACTCTGCCGTTGAATATTGC |
| | TCTGTACTATTTGGTTGCATTCCACAAAACCTCTGACATCCCCAGTACATAC |
| | ATTACTTTACTTGTTTACCTCAATCTAAAGCACAAGCTAGATAATACAAA |
| | (SEQ ID NO: 205) |
| 4-7 | TTTAAACAGCCTGGGGGTTGTTCCCACCCCTGGGGCCCACGTGGCGCTAGT |
| | ACTCTGGTACGCTAGTACCTTTGTACGCCTGTTTTTCCCCTCCCTTAAATAA |
| | ATCAAGGTTGCCACTACTGAGGGGAGTAGTCCGACTCCGCTCCAGCAATGC |
| | TGCACCAGTGCACTGGTACGCTAGTACCTTTTCACGGAGTAGATGGTATCC |
| | CTTACCCCGGAACCTAGAAGATTGCACACAAACCGACCAATAGGCGCACCG |
| | CATCCAGCCGTGCAGCGGTCAAGCACTTCTGTCTCCCCGGTCTGTAAAGAT |
| | CGTTATCCGCCCGACCCACTACGAAAAGCCTAGTAACTGGCCAAGTGAACG |
| | CGAAGTTGCGCTCCGCCACAACCCCAGTGGTAGCTCTGGAAGATGGGGCTC |
| | GCACCACCCCCGTGGTAACACGGTTGCCTGCCCGCGTGTGCTTCCGGGTTC |
| | GGTCTCGTGCCGTTCACTTCAACTTCACGCAACCAGCCAAGAGCCTATTGTG |
| | CTGGGACGGTTTTCCTCCGGGGCCGTGAATGCTGCTAATCCCAACCTCCGA |
| | GCGTGTGCGCACAATCCAGTGTTGCTACGTCGTAACGCGTAAGTTGGAGGC |
| | GGAACAGACTACTTTCGGTACCCCGTGTTTCCTCTCATTTTATTTAATATTTT |
| | ATGGTGACAATTGTTGAGATTTGCGCTCTTGCAACGTTGCCATTGAATATTG |
| | GCTTATACTATTTGGTTGCCTTTTACAAAACCTCTGATATACCCAGTTCTTA |
| | CATTGATCTGCTTGTTTTTCTCAATTTGAAGTATAGACTACAAATAGCAAA |
| | (SEQ ID NO: 206) |
| 4-8 | CCCCCCTCCCCCCCTTCCCTTCCCTTTGCAACGCAACAATTGTAAGTGCCCT |
| | CACCTGTCAATTGGGACCACCACTTTCAGTGACCCCATGCGAAGTGCTGAG |
| | AGAAAGGAAGCTTTCTTACCCTTCATTTGTGAACCCACTGGTCTAAGCCGCT |
| | TGGAATACGATGAGTGGAAAAGTTCATTCTTAATGGAGTGAAACATGCTTA |
| | AATTTCCAGCTCGTGCTGGTCTTTCCAGTACGGGGCGGCCCTGTCTGGCCGT |
| | AATTCTTCAGAGTGTCACGCCACACTTGTGGATCTCACGTGCCACATGACA |
| | GCGCTACAGCTGGAACTGGGTGCTTGGTGCCCATGGAGTAACAGCGAAAA |
| | GTGTTAGATCAAGCCTTGCTTGGGCTATGAGCCTGCGGAACAACAACTGGT |
| | AACAGTTGCCTCAGGGGCCGAAAGCCACGGTGTTAACAGCACCCTCATAGT |
| | TTGATCCACCTCAGGGTGGTGATGTTTAGCAGTTAGTAGTTGCCAATCTGTG |
| | TTCACTGAAATCTCGGCATACCGTGTAGTGTACAGGGGTGAAGGATGCCCA |
| | GAAGGTACCCGTAGGTAACCTTAAGAGACTATGGATCTGATCTGGGGCCTT |
| | GTCCGGAGTGCTTTACACACGGCTCAAGGTTAAAAAACGTCTAGCCCCACA |
| | GAGCCCGAGGGATTCGGGTTTTCCCTTTAAAAACCCGACTAGAGCTTATGG |
| | TGACAATTATTGCTGTTCAGACGAACAGTGTAATTGTTGTCTATTCACAGCA |
| | GTTCTATCAGAGCTTTTCCCACAACGGATCTTCTTGGCAAGCAAATACAGC |
| | AGGAGTCAAT (SEQ ID NO: 207) |
| 4-9 | CACTACGTTACGGTTCCCGCCCGGGACAACTGGTACCCCATTAGGCTACAA |
| | CATGGCTGAAAAGGGTATTGGGTCCCCCCGGATTGTGTCCGTTCGTAGTGT |
| | GTGTAACGTGGTTTACCATCTCCACTAACATTGGACTAAGCATTTCATCTTT |
| | CCTCCCCGATTGTGTACTCACTTGGCTAACGCTGGGTGGTCGCGGTTGGGTC |
| | CTTGATTTACTTTTTCTCGTCTAAGCATTCCGACTGTCCTCCCCGATTATGTG |
| | CTCATTCAGTTAACTGCTGGGTGGTCATGACTAACATCGAGGAACCTTCTGT |
| | CCACGCTTACTTTGAGCTCCGGTCGCTTGACGCTTGTAGGGCGATAGGGTTA |
| | TCTTCCTGACAACATCTTTATTCTACCTCCATAGGCTCTATCTATGGAGACG |
| | GAGTGTGGCACCCGTCCCTTCTTTGGGAGCTTCGGTAGTGACGCCCTTTGTC |
| | ACTCTCGCCAGCCGAGGCATGCCTGGTGCCAGGTAGCAAAGAAAGCATATG |
| | TTTAAGGACTTGACTGATTTAGCGCAAGAGTTTGTAGCGATGTCCATAGTGT |
| | CTGCGGATTCCCCACACGGCGACGTGTGCCGCGGAGGCCAAAAGCCACGGT |
| | GTTCACAGCACCCCTATGGATGCCCACAGACCCCAGTGGGCACTCTTGTTG |
| | CCGGACTTTCAGGAAATTAGGCATAGGCTCTTCTCAAACTCCTGGCATTGG |
| | ACTAGGTAAGAATGCCCCGGAGGTACCCCAGTACTCCTTCGGGAGTCTGGG |
| | ATCTGACCGGGGGCCCCACAAACATGCTTTACGTGTTTCGTGCGGTCAAAA |
| | ATTGTCTAACTAGTCCCAACCTTGAACAAGGGATTGTTCTTTCCTTTTTATT |
| | ACTGAGACTGGCCTATGGTGACAACAGAGATTGACTGTGAATACAGTTATT |
| | TTCTGGTGTTTATCATTTGGTTTTTCTCCGTGCTCTTTTACCTTTGTGGTATTT |
| | GTTCTTTAGATAGGCAAA (SEQ ID NO: 208) |
| 4-10 | CCCGGCCACCCCCTTTCGACGCGGGTACTGCGATAGTGCCACCCCAGTCTTT |
| | CCTACTCCCGACTCCCGACTCTAACCCAGGTTCCTTGGAACAGGAACACCA |
| | ATATACTCATCCCCTGGATGCTGACTAATCAGAGGAACGTCAGCATTTTCC |
| | GGCCCAGGCTAAGAGAAGTAGATAAGTTAGATTCCAAATTGATTTATCATC |
| | CCCTTGACGAATTCGCGTTGGAAATGCACCTCTCACTTGCCGCTCTTCACAC |
| | CCATTAACTTGATTCGGCCTCTGTGTTGAGCCCCTTGTTGAAGTGCTTCCCT |
| | CCATCGTGACGTGGTTGGAGATCTAAGTCAACCGACTCCGACGAAACTACC |
| | ATCATGCCTCCCCGATTATGTGATGCTTTCTGCCCTGCTGGGTGGAGCATCC |
| | TCGGGTTGAGAAAACCTTCTTCCTTTTTCCTTGGACCCCGGTCCCCCGGTCT |
| | AAGCCGCTTGGAATAAGACAGGGTTATCTTCACCTCTTCCTTCTTCTACTTC |
| | ATAGTGTTCTATACTATGAAAGGGTATGTGTCGCCCCTTCCTTCTTTGGAGA |
| | ACACGCGCGGCGGTCTTTCCGTCTCTCGAAAAGCGCGTGTGCGACATGCAG |
| | AGAACCGTGAAGAAAGCAGTTTGCGGACTAGCTTTAGTGCCCACAAGAAA |
| | ACAGCTGTAGCGACCACACAAAGGCAGCGGACCCCCCCTCCTGGCAACAG |
| | GAGCCTCTGCGGCCAAAAGCCACGTGGATAAGATCCACCTTTGTGTGCGGC |
| | ACAACCCCAGTGCCCTGGTTTCTTGGTGACACTTCAGTGAAAACGCAAATG |
| | GCGATCTGAAGCGCCTCTGTAGGAAAGCCAAGAATGTCCAGGAGGTACCCC |
| | TTCCCTCGGGAAGGGATCTGACCTGGAGACACATCACATGTGCTTTACACC |
| | TGTGCTTGTGTTTAAAAATTGTCACAGCTTTCCCAAACCAAGTGGTCTTGGT |
| | TTTCACTCTTTAAACTGATTTCACT (SEQ ID NO: 209) |
| 4-11 | CCCCCGGTTACCCCCTTTCGACGCGGGTACTGCGATAGTGCCACCCCAGTCT |
| | TTCCTACTCCCGACTCCCGACCCTAACCCAGGTTCCTCGGAACAGGAACAC |
| | AAATTTACTCATCCCCTGGATGCTGACTAATCAGAGGAACGTCAGCATTTTC |
| | CGGCCCAGGCTTAGAGAAGTAGATAAGTTAGAATCTAAATTGATATGACTT |
| | CCCCTTGACGAATTCACGTTGGAAATGCACCCCTCACTTGCCGCTCTTCACA |
| | CCCACTAATTGATTCGGCCTACTGTGTTGAGCCCCTTGTTGAAGTGCTTCCC |
| | TCCCTCGTGACGTGGTTGGAGAAATCTTGTCACCCGACTCCGACGAAACTA |
| | CCATCATGCCTCCCCGATTATGTGATGCTTTCTGCCCTGCTGGGTGGAGTAT |
| | CCTCGGGTTGAGAAATCCTTCTTCCTTTTACCTTGGACCTTGGTCCCCCGGT |
| | CTAAGCCGCTTGGAATAAGACAGGGTTATTTTCACCTCTTCTTCTTCTACTT |
| | CATGGTGCTCTATACCATGAAAGGGTATGTGTCGCCCCTTCCTTCTTGGAGA |
| | ACTCACGCGGCGGTCTTCCGTCTCTCAAAAAGCGCGAGTGCGACATGCAGA |
| | GTAACGCGAAGAAAGCAGTTCCTGGCCTAGCTCTAGTGCCCACAAGAAAAC |
| | GGCTGTAGCGACCACACAAAGGCAGCGGAACTCCCCTCCTGGTAACAGGA |
| | GCCTCTGCGGCCAAAAGCCACGTGGATTAGATCCACCTTTGTGTGCGGTGC |
| | AACCCCAGCACCCCGGTTTCTTGTTGACACTCTAGTGAATCCTTGAATGGCA |
| | ATCTCAAGCGCCTCTGTAGGAAAGCCAAGAATGTCCAGGAGGTACCCCTTC |
| | CTCGCGGAAGGGATCTGACCTGGAGACACATCACACGTGCTTTACACTTGT |
| | GCTTGTGTTTAAAAATTGTCACAGCTTTCCCAAACCAAGTGGTCTTGGTTTT |
| | CCTTTTTTATCCTACTGTCAAT (SEQ ID NO: 210) |
| 4-12 | TCCTCACCCATGCTTTTCCTACCCCCACCACGCCCGCATGTTTACTGCTTTCC |
| | TTGATGCTGCCCGTGACTACTTCCATGACCTCCCCAACCCAAACCTCAAACG |
| | CCTTAAGCTATCATGGGCTCTCTATCACCAATCCCCTTCCTTCCCACCCAGA |
| | ACCCCCCCCTCCCTCCCCTACAACGTGTATGAGCAAGACGAGTTTGACAAG |
| | CTCGCCGAAGCCATGCTCACCCACTCTCCCTTCCCCACATCCCATTACCTCT |
| | TCCACCCCCTCCCGTCCAACGCCCGCACCATCGTCGAGCGTGAGGCTGACT |
| | GGGATGGCTGTGATCTAGAAAAGAAATGGCTCGACCTCGTCATAGAGGAC |
| | GATGCCAAGTTCCTTCTGGAGAACGGCTCTCTCCCGTTTGGCTCCACCCTTG |
| | CC (SEQ ID NO: 211) |
| 4-13 | TTCTCGCCTGAGTCAACAAAGCGAGAAACCTGCCCCTCCAGCGCCAGACGA |
| | GCGGCATAAAACTTGAACTTCTGGCATGCTCCACCACCCTTTTCCCCATTCC |
| | AACCCCCATTGCGCTCTCAAGGTCGCGCTTTTTCGAGACTAGCTCGGATTCA |
| | AAAGTTCCTGGCACCCTTTGCCCCTTCAGGCCCTTAAGGTAGGAACTGACCT |
| | TGTGCTGTGCCCTCGGTGCGGAAGTGCTACTGCGTAGCGATTGTAAGATCC |
| | CTTTGTGGTTCTGCCCTGGCAAGGTTATAGAGTACTGTGATCCGCTGCGGAT |
| | GCCATCCTGGTAACAGGACCCCCAGTGTGCGCAACAGTATGTTCACGGTCT |
| | TCCGTGTCCACCACATTCGGAACACTGCTCTCGTGAAACAGTGTGTGTCCA |
| | ATCCCTGCAATCAGTATCAACTACACCACCTAGGAATGCTAGGAAGGTACC |
| | CCGGTCCGCCGGGATCTGATCCTAGGCTAATTGTCTACGGTGGTGCTCCTTT |
| | TTATTTTCCACTTCAATTCATTGGTTACAACTGCTCGATCCCTGTGTTTGCTG |
| | CCCTTCTCTGCTCTCATCGCCATTCTCAAGTGTTCACACTGTCCAAGTTCCTT |
| | TGGTTGTTCGCTTCCACTTGCCACTGTCAACTCTTGTC (SEQ ID NO: 212) |
| 4-14 | TCACCCTCTTTTCCGGTGGTCCGGACCCAGACCACCGTTACTCCATTCAGCT |
| | TCTTCGGAACCTGTTTGGAGGAATTAAACGGGCACCCACCCACCTTCACCC |
| | CCTTTTCGTAACTAAGTGTGTGCCCAATCTCATGACTCCTGCTGACTTCACC |
| | GACCAGCAGTGTCCAAAACGCTAGGTGAATTTCCTTCCTCCCCCTCTGGGCT |
| | TCTGCCCAGCTCCCTCCCTCCAGCCTGACGTGCCACAGGCTGTGCAAAGAC |
| | CCCGCGAAAGCTGCCAAAAGTGGCAATTGTGGGTCCCCCCTTTGTAAAGGC |
| | GTCGAGTCTTTCTCCCTTAAGGCTAGACCCGTCAGTGAATTCTGTCGGGCAA |
| | CTAGTGACGCCACTGCATGCCTCCGACCTCGGCCGCGGAGTGCTGCCCCCC |
| | AAGTCGTGCCCCTGACTACAAGTTGTGCTGTCTGGCAAACATTGTCTGTGA |
| | GAATGTTCCGCTGTGGCTGCCAAGCCTGGTAACAGGCTGCCCCAGTGTGCG |
| | TAGTTCTCATCCAGACTTCGGTCTGGCAACTTGCTGTTAAGACACGGCGTAA |
| | GGGGCGTGTGCCAACGCCCTGGAACGAGTGTCCACTCTAATACCCCGAGGA |
| | ATGCTACGCAGGTACCCCTGGCTCCCCAGGGATCTGAGCGTAGGCTAATTG |
| | TCTAAGGGTATTTTCATTTCCCACTCTTTCTTTCTTGTTCATA (SEQ ID NO: |
| | 213) |
| 4-15 | TCTGTCCTCACCCCATCTTCCCTTCTTTCCTGCACCGTTACGCTTACTCGCAT |
| | GTGCATTGAGTGGTGCACGTGCTTGAACAAACAGCTACACTCACATGGGGG |
| | CGGGTTTTCCCGCCCTGCGGCCTCTCGCGAGGCCCACCCCTCCCCTTCCTCC |
| | CATAACTACAGTGCTTTGGTAGGTAAGCATCCTGATCCCCCGCGGAAGCTG |
| | CTCACGTGGCAACTGTGGGGACCCAGACAGGTTATCAAAGGCACCCGGTCT |
| | TTCCGCCTTCAGGAGTATCCCTGCTAGTGAATTCTAGTAGGGCTCTGCTTGG |
| | TGCCAACCTCCCCCAAATGCGCGCTGCGGGAGTGCTCTTCCCCAACTCACC |
| | CTAGTATCCTCTCATGTGTGTGCTTGGTCAGCATATCTGAGACGATGTTCCG |
| | CTGTCCCAGACCAGTCCAGTAATGGACGGGCCAGTGTGCGTAGTCGTCTTC |
| | CGGCTTGTCCGGCGCATGTTTGGTGAACCGGTGGGGTAAGGTTGGTGTGCC |
| | CAACGCCCGTACTTTGGTGATACCTCAAGACCACCCAGGAATGCCAGGGAG |
| | GTACCCCGCTTCACAGCGGGATCTGACCCTGGGCTAATTGTCTACGGTGGTT |
| | CTTCTTGCTTCCACTTCTTTCTACTGTTCATG (SEQ ID NO: 214) |
| 4-16 | ATTCTCGGGCTACGGCCCTGGAGCCACTCCGGCTCCTAAAGATTTAGAAGT |
| | TTGAGCACACCCGCCCACTAGGGCCCCCCATCCAGGGGGGCAACGGGCAA |
| | GCACTTCTGTTTCCCCGGTATGATCTGATAGGCTGTAACCACGGCTGAAAC |
| | AGAGATTATCGTTATCCGCTTCACTACTTCGAGAAGCCTAGTAATGATGGG |
| | TGAAATTGAATCCGTTGATCCGGTGTCTCCCCCACACCAGAAACTCATGAT |
| | GAGGGTTGCCATCCCGGCTACGGCGACGTAGCGGGCATCCCTGCGCTGGCA |
| | TGAGGCCTCTTAGGAGGACGGATGATATGGATCTTGTCGTGAAGAGCCTAT |
| | TGAGCTAGTGTCGACTCCTCCGCCCCCGTGAATGCGGCTAATCCTAACCCC |
| | GGAGCAGGTGGGTCCAATCCAGGGCCTGGCCTGTCGTAATGCGTAAGTCTG |
| | GGACGGAACCGACTACTTTCGGGAAGGCGTGTTTCCATTTGTTCATTATTTG |
| | TGTGTTTATGGTGACAACTCTGGGTAAACGTTCTATTGCGTTTATTGAGAGA |
| | TTCCCAACAATTGAACAAACGAGAACTACCTGTTTTATTAAATTTACACAG |
| | AGAAGAATTACA (SEQ ID NO: 215) |
| 4-17 | GTGGCCACGCCCGGGCCACCGATACTTCCCTTCACTCCTTCGGGACTGTTGG |
| | GGAGGAACACAACAGGGCTCCCCTGTTTTCCCATTCCTTCCCCCTTTTCCCA |
| | ACCCCAACCGCCGTATCTGGTGGCGGCAAGACACACGGGTCTTTCCCTCTA |
| | AAGCACAATTGTGTGTGTGTCCCAGGTCCTCCTGCGTACGGTGCGGGAGTG |
| | CTCCCACCCAACTGTTGTAAGCCTGTCCAACGCGTCGTCCTGGCAAGACTAT |
| | GACGTCGCATGTTCCGCTGCGGATGCCGACCGGGTAACCGGTTCCCCAGTG |
| | TGTGTAGTGCGATCTTCCAGGTCCTCCTGGTTGGCGTTGTCCAGAAACTGCT |
| | TCAGGTAAGTGGGGTGTGCCCAATCCCTACAAAGGTTGATTCTTTCACCAC |
| | CTTAGGAATGCTCCGGAGGTACCCCAGCAACAGCTGGGATCTGACCGGAGG |
| | CTAATTGTCTACGGGTGGTGTTTCCTTTTTCTTTTCACACAACTCTACTGCTG |
| | ACAACTCACTGACTATCCACTTGCTCTGTCACG (SEQ ID NO: 216) |
| 4-18 | TTTGCTCAGCGTAACTTCTCCGGGTTACGTGGAGACCAAAAGGCTACGGAG |
| | ACTCGGGCTACGGCCCTGGAGCACCTAGGTGCTCCTAAAGACGTTAGAAGT |
| | TGTACAAACTCGCCCAATAGGGCCCCCCAACCAGGGGGGTAGCGGGCAAG |
| | CACTTCTGTTTCCCCGGTATGATCTCATAGGCTGTACCCACGGCTGAAAGAG |
| | AGATTATCGTTACCCGCCTCACTACTTCGAGAAGCCCAGTAATGGTTCATG |
| | AAGTTGATCTCGTTGACCCGGTGTTTCCCCCACACCAGAAACCTGTGATGG |
| | GGGTGGTCATCCCGGTCATGGCGACATGACGGACCTCCCCGCGCCGGCACA |
| | GGGCCTCTTCGGAGGACGAGTGACATGGATTCAACCGTGAAGAGCCTATTG |
| | AGCTAGTGTTGATTCCTCCGCCCCCGTGAATGCGGCTAATCCCAACTCCGG |
| | AGCAGGCGGGCCCAAACCAGGGTCTGGCCTGTCGTAACGCGAAAGTCTGG |
| | AGCGGAACCGACTACTTTCGGGAAGGCGTGTTTCCTTTTGTTCCTTTTATCA |
| | AGTTTTATGGTGACAACTCCTGGTAGACGTTTTATTGCGTTTATTGAGAGAT |
| | TTCCAACAATTGAACAGACTAGAACCACTTGTTTTATCAAACCCTCACAGA |
| | ATAAGATAACA (SEQ ID NO: 217) |
| 4-19 | CCCCCCTCCCCCCCTTCCCTTCCCTTTGCAACGCAACAATTGTAAGTGCCCT |
| | CACCTGTCAATTGGGACCACCACTTTCAGTGACCCCATGCGAAGTGCTGAG |
| | AGAAAGGAAGCTTTCTTACCCTTCATTTGTGAACCCACTGGTCTAAGCCGCT |
| | TGGAATACGATGAGTGGAAAAGTTCATTCTTAATGGAGTGAAACATGCTTA |
| | AATTTCCAGCTCGTGCTGGTCTTTCCAGTACGGGGCGGCCCTGTCTGGCCGT |
| | AATTCTTCAGAGTGTCACGCCACACTTGTGGATCTCACGTGCCACATGACA |
| | GCGCTACAGCTGGAACTGGGTGCTTGGTGCCCATGGAGTAACAGCGAAAA |
| | GTGTTAGATCAAGCCTTGCTTGGGCTATGAGCCTGCGGAACAACAACTGGT |
| | AACAGTTGCCTCAGGGGCCGAAAGCCACGGTGTTAACAGCACCCTCATAGT |
| | TTGATCCACCTCAGGGTGGTGATGTTTAGCAGTTAGTAGTTGCCAATCTGTG |
| | TTCACTGAAATCTCGGCATACCGTGTAGTGTACAGGGGTGAAGGATGCCCA |
| | GAAGGTACCCGTAGGTAACCTTAAGAGACTATGGATCTGATCTGGGGCCTT |
| | GTCCGGAGTGCTTTACACACGGCTCAAGGTTAAAAAACGTCTAGCCCCACA |
| | GAGCCCGAGGGATTCGGGTTTTCCCTTTAAAAACCCGACTAGAGCTTATGG |
| | TGACAATTATTGCTGTTCAGACGAACAGTGTAATTGTTGTCTATTCACAGCA |
| | GTTCTATCAGAGCTTTTCCCACAACGGATCTTCTTGGCAAGCAAATACAGC |
| | AGGAGTCAAT (SEQ ID NO: 207) |
| 4-20 | GTGGCCACGCCCGGGCCACCGATACTTCCCTTCACTCCTTCGGGACTGTTGG |
| | GGAGGAACACAACAGGGCTCCCCTGTTTTCCCATTCCTTCCCCCTTTTCCCA |
| | ACCCCAACCGCCGTATCTGGTGGCGGCAAGACACACGGGTCTTTCCCTCTA |
| | AAGCACAATTGTGTGTGTGTCCCAGGTCCTCCTGCGTACGGTGCGGGAGTG |
| | CTCCCACCCAACTGTTGTAAGCCTGTCCAACGCGTCGTCCTGGCAAGACTAT |
| | GACGTCGCATGTTCCGCTGCGGATGCCGACCGGGTAACCGGTTCCCCAGTG |
| | TGTGTAGTGCGATCTTCCAGGTCCTCCTGGTTGGCGTTGTCCAGAAACTGCT |
| | TCAGGTAAGTGGGGTGTGCCCAATCCCTACAAAGGTTGATTCTTTCACCAC |
| | CTTAGGAATGCTCCGGAGGTACCCCAGCAACAGCTGGGATCTGACCGGAGG |
| | CTAATTGTCTACGGGTGGTGTTTCCTTTTTCTTTTCACACAACTCTACTGCGT |
| | ACAACTCACTGACTATCCACTTGCTCTGTCACG (SEQ ID NO: 218) |
| 4-21 | TTTGCTCAGCGTAACTTCTCCGGGTTACGTGGAGACCAAAAGGCTACGGAG |
| | ACTCGGGCTACGGCCCTGGAGCACCTAGGTGCTCCTAAAGACGTTAGAAGT |
| | TGTACAAACTCGCCCAATAGGGCCCCCCAACCAGGGGGGTAGCGGGCAAG |
| | CACTTCTGTTTCCCCGGTATGATCTCATAGGCTGTACCCACGGCTGAAAGAG |
| | AGATTATCGTTACCCGCCTCACTACTTCGAGAAGCCCAGTAATGGTTCATG |
| | AAGTTGATCTCGTTGACCCGGTGTTTCCCCCACACCAGAAACCTGTGATGG |
| | GGGTGGTCATCCCGGTCATGGCGACATGACGGACCTCCCCGCGCCGGCACA |
| | GGGCCTCTTCGGAGGACGAGTGACATGGATTCAACCGTGAAGAGCCTATTG |
| | AGCTAGTGTTGATTCCTCCGCCCCCGTGAATGCGGCTAATCCCAACTCCGG |
| | AGCAGGCGGGCCCAAACCAGGGTCTGGCCTGTCGTAACGCGAAAGTCTGG |
| | AGCGGAACCGACTACTTTCGGGAAGGCGTGTTTCCTTTTGTTCCTTTTATCA |
| | AGTTTTATGGTGACAACTCCTGGTAGACGTTTTATTGCGTTTATTGAGAGAT |
| | TTCCAACAATTGAACAGACTAGAACCACTTGTTTTATCAAACCCTCACAGA |
| | ATAAGATAACA (SEQ ID NO: 217) |
| 4-22 | CACTATTTAGAGAAAGTAGGAGTCTGGATTCCCAAGGCCCACTCGGTTCAA |
| | CTTCGGTTTCCGGACAAATACAAAGAACCTCAGTCCTCTTGGTACTTTCTCG |
| | CCTGAGTCTACAAAGCGAGAAACCTGCCCCTCTAACGCCAGACGAGCGGCA |
| | TAAAACTCGAACTTCTGGCACGTTCCACCACCCCTTCTCCTATCCCAACCCC |
| | CATTGCGCTCTCAAGGTCGCGCTTTTCCGAGACTAGCTCGGATTCAAAAAG |
| | TTCCTGGCACCCTTTACCCCTTCAGGCCCTTAAGGTAGGAACTGACCTTGTG |
| | CTGTGATCTCGGTGCGGAAGTGCTACTGCGTAGTGATTGTAAGATCCTTTTG |
| | TGGTTCTGCCCTGGCAAGGCTACAGAGTGCTGTGATCCGCTGCGGATGCCA |
| | TCCTGGTAACAGGACCCCCAGTGTGCGCAACAGTATGTTCACGGTCTTCCG |
| | TGTTCACCACATTCGGAACACTGCTTTCGTGAAACAGTGTGTGTCCAATCCC |
| | TGTGATCAGTATCAACCACACCACCTAGGAATGCTAGGAAGGTACCCCGGT |
| | TCGCCGGGATCTGATCCTAGGCTAATTGTCTACGGTGGTGCTCCTTTTTATT |
| | TTCCACTTCAACTCACTGGTTACAACTGCTTGATTTCTGTGTTTGCTGCTTTT |
| | CTCTGCTCTCACTGCCATTCTCAAGTGTTCACACTGTCCAAGCTCCTTTGGTT |
| | GTTCGCTTCCACTTGCCACTGTCAACTCTTGTC (SEQ ID NO: 219) |
| 4-23 | CAACAGGACATACGATGCCGACAGTAAAACAGCTCTGGGGTTGTTCCCACC |
| | CCAGAGGCCCACGTGGCGGCTAGTACTCTGGTATCGCGGTACCTTTGTACG |
| | CCTGTTTTATACCCCTACCCCATGTAACTTTAGAAGCAGTTCAACAAGTTCA |
| | ATAGGGGGGGTGCAAACCAGCACCACCACGAACAAGCACTTCTGTAACCC |
| | CGGTGACCTTGTATAAGCTGTACCCACGGCTGAAAGCGAGTGATCCGTTAT |
| | CCGCTTAAGTACTTCGAGAAGCCTAGTATCACCTTAGAATCTTCGATGCGTT |
| | GCGCTCAGCACTCTATCCCGAGTGTAGCTTAGGCTGATGAGTCTGGGCGTT |
| | CCTCACCGGCGACGGTGGCCCAGGCTGCGTTGGCGGCCTACCCATGGCTAA |
| | CGCCATGGGACGCTAGATGTGAACAAGGTGTGAAGAGCCTATTGAGCTACT |
| | TAAGAGTCCTCCGGCCCCTGAATGCGGCTAATCCCAACCACGGAGCAGGTG |
| | CCTTCAACCCAGAGGGTGGCCTGTCGTAACGCGCAAGTCTGTGGCGGAACC |
| | GACTACTTTGGGTGTCCGTGTTTCCTTTTATCTTTTATATGGCTGCTTATGGT |
| | GACAATCATAGATTGTTATCATAAAGCGACTTGGATTGGCCATCCGGTAAA |
| | ATACAAACACATCATTTACTTGTTTGTTGGATTTACTCCACTCTCCCAATTT |
| | ACTCCTAGCATAATATCCATTGTTTTGTTAACAAGACATTACTATCACA |
| | (SEQ ID NO: 220) |
| 4-24 | GAACGTAGCGGCTACACTACTTAAACAAGCCTGTGGGTTGTTCCCACCCAC |
| | AGGGCCCACTGGGCGCTAGCACACTGATTCTGCGGGATCTTTGTGCGCCTG |
| | TTTTATAACCCCTTCCCTAAGCAGCAACTTAGAAGCTTCACACTATCACGAC |
| | CAGCAGTGGGCGTGACGCACCAGTCACGTCTTGGTCAAGCACTTCTGTTTC |
| | CCCGGACTGAGTATCAATAGACTGCTCATGCGGTTGAAGGAGAAAACGTTC |
| | GTTATCCGGCTAACTACTTCGAGAAACCTAGTAGCACCGTGAAAGTTGCGG |
| | AGTGTTTCGCTCAGCACTTCCCCCGTGTAGATCAGGTCGATGAGTCACTGTA |
| | AACCCCACGGGCGACCGTGACAGTGGCTGCGTTGGCGGCCTGCCCATGGGG |
| | AAACCCATGGGACGCTCTAATACAGACACGGTGTGAAGAGTCTATTGAGCT |
| | AGTTAGTAGTCCTCCGGCCCCTGAATGCGGCTAATCCTAACTGCGGAGCGC |
| | GTACCCTCAACCCAGAGGGCGGCGCGTCGTAATGGGTAACTCTGCAGCGGA |
| | ACCGACTACTTTGGGTGTCCGTGTTTCCTTTTTATTCCTTATTGGCTGCTTAT |
| | GGTGACAATTGAAAAGTTGTTACCATATAGCTATTGGATTGGCCATCCAGT |
| | GACCAACAGAGCTATTGTTTACCTGTTTATTGGATACGTCCCTCTTAATCTC |
| | AAGACCATCCAAACTCTTGATTATATATTACTCCTTAATCATAAGAA (SEQ |
| | ID NO: 221) |
| 4-25 | CGAGCTCCGTGAATAAACTCCTTCACTTTCCATAACTACAGTGCTTTGGTAG |
| | GTAAGCATCCTGACCCCCCGCGGAAGCTGCCAACGTGGCAACTGTGGGGAT |
| | CCAGGCAGGTTATCAAAGGCACCCGGTCTTTCCGCCTTCAGGAGTATCCCT |
| | GCCGGTGAATTCCGACAGGGCTCTGCTTGGTGCCAACCTCCCCCAAATGCG |
| | CGCTGCGGGAGTGCTCTTCCCCAACTCATCTTAGTAACCTCTCATGTGTGTG |
| | CTTGGTCAGCATATCTGAGGCGACGTTCCGCTGTCCCAGACCAGTCCAGCA |
| | ATGGACGGGCCAGTGTGCGTAGTCGCTTTCCGGTTTCCCGGCGCATGTTTGG |
| | CGAAACGCTGAGGTAAGGTTGGTGTGCCCAATGCCCGTAATTTGGTGACAC |
| | CTCAAGACCACCCAGGAATGCCAGGGAGGTACCCCACTTCGGTGGGATCTG |
| | ACCCTGGGCTAATTGTCTACGGTGGTTCTTCTTGCTTCCACTTCTCTTTTTTC |
| | TGGCATG (SEQ ID NO: 222) |
| 4-26 | AGGAGTGCTGATATAGATTCGGATTTAGGCCGTGGTGCCCGTGGGCACGAC |
| | GGCCACGGCTCCAGCCGCACTCCTCTCCACGCCTTGTCCCACGGGACATAA |
| | AGTGGAGCCCTAAGCCCTAAACCCCCATGTTACCCAATTTTCTTATCCACTT |
| | CCCTTTTTCCCTTTGACTCACATTTTTCTTGACCGCGCTTGTGAGGCGGTTCC |
| | CGTACAGTTGAGTTGACGGGTCCCGCGCGGGACCTGAAAATTACTCCATAC |
| | TGATTAGGAAAGGTTCTAGTACGCTTGTACCCTTAAGGCCAGCGGGCTACC |
| | TCCTTTAGGTCTTATAGCCCCAACTGGTTAGAACCACTCCAGCTCACCTCTA |
| | TCGGTGCGGGAGTGCTACCACCCCTGCCTGCGTCAAGCCCGTTTTTCTAGTG |
| | ATCTGTCCACAAACACTTGGATTCCCGTCGCGACTGACAGAACCTGGCGAC |
| | AGGTGTCTCGATGTGTGTGACCAAGTGGCATGAGAATCGGAAACTAACGCT |
| | GGTAAGATGGTAAGTGCCCAATCCCTGTGGGGTGATGTTTTTCTCTGCATAG |
| | GAATGCTGCGAAGGTACCCCGGCCTTGAGTCGGGATCTGATCGCAGGCTAA |
| | TTGTCCTCTGTGCAGTTCATTTCCTTTTCTTTTCATTGGCATC (SEQ ID NO: |
| | 223) |
| 4-27 | GTCCACTGAGACATCTTACTCACCCACTCACAGGACCCACGCGGTGCTAGC |
| | ACTCTGGTTCCACGGGACCTTTGTGCGCCTGTTTTATGTCCCCTCCCCAATTT |
| | GTAACTTAGAAGCAATTTACAACACTGATCAGTAGCAGGCATAGCGCACCA |
| | GCTATGTCTTGATCAAGCACTTCTGTTTCCCCGGACCGAGTATCAATAGACT |
| | GTTCACACGGTTGAAGGAGAAAGCGTCCGTTATCCGGCTAACTACTTCGAG |
| | AAACCCAGTAGCACCATTGAAACTACAGAGTGTTTCGCTCCACACTTCCCC |
| | CGTGTAGATCAGGTCGATGAGTCACTGCAATCCCCACGGGCGACCGTGGCA |
| | GTGGCTGCGCTGGCGGCCTGCCTATGGGGCAACCCATAGGACGCTCTAATG |
| | TGGACATGGTGTGAAGAGTCTATTGAGCTAGTTAGTAGTCCTCCGGCCCCT |
| | GAATGCGGCTAATCCTAACTGCGGAGCACATGCCTTCAATCCAGAGGGTAG |
| | TGTGTCGTAATGGGCAACTCTGCAGCGGAACCGACTACTTTGGGTGTCCGT |
| | GTTTCCTTTTATTCTCACATTGGCTGCTTATGGTGACAATTACAGAATTGTT |
| | ACCATATAGCTATTGGATTGGCCATCCGGTGTGCAATAGAGCTATTATATA |
| | CCTATTTGTTGGCTTTGTACCACTGACCTTAAAATCTTTAACCACTCTTGAG |
| | TGTATATTAACCCTTAATACAATCAAAC (SEQ ID NO: 224) |
| 4-28 | TCCGAGGAACAGCATAAACATAAAACAGCCTTGGGGTTGTTCCCACTCCAA |
| | GGGCCCACGTGGCGGCTAGTACTCTGGTACTTCGGTACCTTTGTACGCCTGT |
| | TTTATCTCCCTTCCCAATGTAATTTAGAAGCTCTTAAATCAAGGCTCAATAG |
| | GTGGGGCGCAAACCAGCGCTCTCATGAGCAAGCACTCCTGTCTCCCCGGTG |
| | TGGTTGTATAAACTGTTCCCACGGTTGAAAACAACCTATCCGTTATCCGCTA |
| | TAGTACTTCGAGAAACCTAGTATCACCTTTGGATTGTTGACGCGTTGCGCTC |
| | AGCACACTAACCCGTGTGTAGCTTGGGTCGATGAGTCTGGACACACCCCAC |
| | TGGCGACAGTGGTCCAGGCTGCGTTGGCGGCCTACTCATGGTGAAAACCAT |
| | GAGACGCTAGACATGAACAAGGTGTGAAGAGTCTATTGAGCTACTATAGA |
| | GTCCTCCGGCCCCTGAATGCGGCTAATCCTAACCATGGAGCAAGTGCTCAC |
| | AGGCCAGTGAGTTGCTTGTCGTAATGCGCAAGTCCGTGGCGGAACCGACTA |
| | CTTTGGGTGTCCGTGTTTCACTTTTTACTTTTATGACTGCTTATGGTGACAAT |
| | TTGATATTGTTACCATTTAGCTTGTCAAATTAATTGCAAAGGATCCTAAATC |
| | TTACTTACCAATTTGCATTTTGACAATCTTGATTTGAAAATTTTAATA (SEQ |
| | ID NO: 225) |
| 4-29 | TTAAAACAGCCTGTGGGTTGTGACCCACCCACAGGGCCCACTGGGCGCTAG |
| | CACTCTGATTCTACGGAATCCTTGTGCGCCTGTTTTATGTCCCTTCCCCCAAT |
| | CAGTAACTTAGAAGCATTGCACCTCTTTCGACCGTTAGCAGGCGTGGCGCA |
| | CCAGCCATGTCTTGGTCAAGCACTTCTGTTTCCCCGGACCGAGTATCAATAG |
| | ACTGCTCACGCGGTTGAGGGAGAAAACGTCCGTTACCCGGCTAACTACTTC |
| | GAGAAGCCTAGTAGCACCATGAAAGTTGCAGAGTGTTTCGCTCAGCACTTC |
| | CCCCGTGTAGATCAGGTCGATGAGTCACTGCGATCCCCACGGGCGACCGTG |
| | GCAGTGGCTGCGTTGGCGGCCTGCCTGTGGGGTAACCCACAGGACGCTCTA |
| | ATATGGACATGGTGCAAAGAGTCTATTGAGCTAGTTAGTACTCCTCCGGCC |
| | CCTGAATGCGGCTAATCCTAACTGCGGAGCACATACCCTCGACCCAGGGGG |
| | CAGTGTGTCGTAACGGGCAACTCTGCAGCGGAACCGACTACTTTGGGTGTC |
| | CGTGTTTCCTTTTATTCTTATACTGGCTGCTTATGGTGACAATTGAAAGATT |
| | GTTACCATATAGCTATTGGATTGGCCATCCGGTGTGCAACAGAGCTATTATT |
| | TACCTATTTGTTGGGTATATACCACTCACATCCAGAAAAACCCTCGACACA |
| | CTAGTATACATTCTTTACTTGAATTCTAGAAA (SEQ ID NO: 226) |
| 4-30 | TTAAAACAGCTCTGGGGTTGTTCCCACCCCAGAGGCCCACGTGGCGGCTAG |
| | TACTCCGGTATCACGGTACCCTTGTACGCCTGTTTTACACTCCCTTCCCCCG |
| | TAACTTAGAAGAAACAAACAAAGTTCAATAGAAGGGGGTACAAACCAGTA |
| | CCACCACGAACAAGCACTTCTGTTTCCCCGGTGACGTTGTATAGACTGTACC |
| | CACGGTCGAAAACGATTGATCCGTTATCCGCTTTTGTACTTCGAGAAGCCTA |
| | GTATCATCTTGGAATCTTCGATGCGTTGCGCTCAGCACTCAATCCCAGAGTG |
| | TAGCTTAGGTCGATGAGTCTGGACGTTCCTCACCGGCGACGGTGGTCCAGG |
| | CTGCGTTGGCGGCCTACCTGTGGCCCAAAGCCACAGGACGCTAGTTGTGAA |
| | CAAGGTGTGAAGAGCCTATTGAGCTACAAGAGAGTCCTCCGGCCCCTGAAT |
| | GCGGCTAATCCTAACCACGGAGCAAGGGTGTGTGAGCCAACACATACCTTG |
| | TCGTAATGCGTAAGTTCGTGGCGGAACCGACTACTTTGGGTGTCCGTGTTTC |
| | CTTTTATTCTTGTATTGGCTGCTTATGGTGACAATCATAGATTGTTATCATA |
| | AAGCGACTTGGATTGGCCATCCAGTGGAAGTGAAATACCTTGTCTACCTGT |
| | CTGTTGGTTTTACTCCATTTGACTAATTCACTTACAAGCTTATTGCTATAATT |
| | TTTATTGACTAAACAACAATACTACA (SEQ ID NO: 227) |
| 4-31 | CGTAGGTCCACATATTGTTGCTGTTTTCCCATTCCTTCCCCCTTTTCCCAACC |
| | CCAACCGCCGTATCTGGTGGCGGCAAGACACACGGGTCTTTCCCTCTAAAG |
| | CACAATTGTGTGTGTGTCCCAGGTCCTCCTGCGTACGGTGCGGGAGTGCTCC |
| | CACCCAACTGTTGTAAGCCTGTCCAACGCGTCGTCCTGGCAAGACTATGAC |
| | GTCGCATGTTCCGCTGCGGATGCCGACCGGGTAACCGGTTCCCCAGTGTGT |
| | GTAGTGCGATCTTCCAGGTCCTCCTGGTTGGCGTTGTCCAGAAACTGCTTCA |
| | GGTAAGTGGGGTGTGCCCAATCCCTACAAAGGTTGATTCTTTCACCACCTTA |
| | GGAATGCTCCGGAGGTACCCCAGCAACAGCTGGGATCTGACCGGAGGCTA |
| | ATTGTCTACGGGTGGTGTTTCCTTTTTCTTTTCACACAACTCTACTGCTGACA |
| | ACTCACTGACTATCCACTTGCTCTCTTGTGCCTTTCTGCTCTGGTTCAAGTTC |
| | CTTGATTGTTTTTGACTGCTTTTCACTGCTTTTCTTCTCACAATCCTTGCTCA |
| | GTTCAAAGTC (SEQ ID NO: 228) |
| 4-32 | ACCACCGTGAATGCGACAATGGCGCGCCTCGGCAAGGCCGCCGATACCGC |
| | ACTGTGCTACTTCGGTACCACTCTGCGGGAATGACAATATGCCCGGTGTAT |
| | AGTTTGCCTGTTTCTCGCACCGTTACCGCTCGTTCGGGAATGTGAAACTGGC |
| | ACCCCTCCTCTCCCCTACCACCCTTTCTCCTTCGCCCCCATTCATAATTTACA |
| | ACGCCGCACACAGCGGCGGCCGCCAAGGGCTAGCCTGGCGGTTATAAAAG |
| | GAACCTGGGTCTTTCCCTCTTCAAGCCAAAAGGTAGGTTCCCTGTGTCCCTG |
| | AATGCTCGGTGAGGAATGCTGCACCGTAACGCTTTGTGAAGTGTTTGCAAG |
| | TTCTGGCCCGGCAAGCCTACAGAGTGCTGTGATCCGCTGCGGACGCCATCC |
| | TGGTAACAGGACCCCCAGTGTGCGCAACAGTATGTTCAGACTTCGGTTTGT |
| | TCACTTGCTTTCATGGACCATTGCGCGAAAGTGCGTGCGCCATATCCCTGTA |
| | CTTCAGGTGTGCTTCTCTGGACCCTAGGAATGCTGCGAAGGTACCCCGTTTC |
| | GGCGGGATCTGATCGCAGGCTAATTGTCTATGGGTTCAGTTTCCTTTTTCTT |
| | TACTCCACAATTGACTGCTTAACTGACTCTGGATCTTGTGCTTCCACTGCTC |
| | TTTCTGCTCTCAAAACGGCTTCACTTACCAACTCTCACCTTTCGACCAACAC |
| | CATTTACACACTAACTTTTTTCGACTCTTCTGACTCCTGGCTTGGTGAAGAC |
| | (SEQ ID NO: 229) |
| 4-33 | TGAGGGAGATACTAACCCTGCCACCCTTTCTCCTTCGCCCCCATTCATAATT |
| | TACAACGCCGCACACAGCGGCGGCCGCCAAGGGCTAGCCTGGCGGTTATA |
| | AAAGGAACCTGGGTCTTTCCCTCTTCAAGCCAAAAGGTAGGTTCCCTGTGT |
| | CCCTGAATGCTCGGTGAGGAATGCTGCACCGTAACGCTTTGTGAAGTGTTT |
| | GCAAGTTCTGGCCCGGCAAGCCTACAGAGTGCTGTGATCCGCTGCGGACGC |
| | CATCCTGGTAACAGGACCCCCAGTGTGCGCAACAGTATGTTCAGACTTCGG |
| | TTTGTTCACTTGCTTTCATGGACCATTGCGCGAAAGTGCGTGCGCCATATCC |
| | CTGTACTTCAGGTGTGCTTCTCTGGACCCTAGGAATGCTGCGAAGGTACCCC |
| | GTTTCGGCGGGATCTGATCGCAGGCTAATTGTCTATGGGTTCAGTTTCCTTT |
| | TTCTTTACTCCACAATTGACTGCTTAACTGACTCTGGATCTTGTGCTTCCACT |
| | GCTCTTTCTGCTCTCAAAACGGCTTCACTTACCAACTCTCACCTTTCGACCA |
| | ACACCATTTACACACTAACTTTTTTCGACTCTTCTGACTCCTGGCTTGGTGA |
| | AGAC (SEQ ID NO: 230) |
| 4-34 | ATCGAAAGGCTTATACGATCCCACGTGTGTATGCCAACCTACATGGACTCG |
| | TGCATGTAGGTTGGATTCACAACCCTTTCATAACCCCCCCCTTTTAACCCAA |
| | CCCTTCGTAACCGTACGCTTCACTCGCCTTTGGGTATAGCGGCCCAATGTGC |
| | TGAAGAAAAGGATACGCTATAAGGGGCCAACGGGTGGTGGCCCTTAAGAC |
| | CACCCAACCTAGAAGCTTGTACACTCGGGCAATAGTGAGGCCCACATCCAG |
| | TGGGTCAAGCCCAAAGCATTCTTGTTCCCCGGTATGATCTCATAAGCTGTAC |
| | CCACGGCTGAAAGAGTGATTATCGTTATCCCACTCAGTACTTCGGAGAGCC |
| | TAGTACACCACTTGGAAATGGAAGTCTGTGATCCGGGGTTGACCCTGAACC |
| | CCAGAAACTCATGATGAGGCTAACCTTCCCGAACACGGCGACGTGTGGTTA |
| | GCCTGCGCTGGCATGAGGCCTCTTTGTAGGCAGACTGAAATGGAAGGGTGA |
| | CGAAGAGCCGACTGAGCTACTGTTTTATTCCTCCGGCCCCCTGAATGCGGCT |
| | AATCCTAACTCCTGGTCCAGTACTTGTAACCCAACAGGTGGCTGGTCGTAA |
| | TGCGTAAGCCGGGAGCGGAACCGACTACTTTGGGGCGTCCGTGTTTCTCAA |
| | TATTATTCATTTCTAGCTTATGGTGACAATTTATGATTGCAGAGATTGTGCT |
| | GTATTTGTGTCTGAGAGAAGAAGTAACAAT (SEQ ID NO: 231) |
| 4-35 | TCGGTGTCAGGCCTCACCTTTTAAAACAGCCTGTGGGTTGTTCCCACCCACA |
| | GGGCCCACTGGGCGCTAGCACTCTGATTTCACGAAATCTTTGTGCGCCTGTT |
| | TTATATCCCTTCCCCCAGTCTGAAACATAGAAGCAATGCGCATCACTGATC |
| | AATAGCAGGCGTAACGCGCCAGTTACGTCATGATCAAGCACATCTGTCTCC |
| | CCGGACCGAGTATCAATAGACTGCTTGCGCGGTTGAAGGAGAAACAGTTCG |
| | TTACCCGGCTAACTACTTCGAGAAGCCCAGTAGCACCATGGAAGCTGCAGG |
| | GTGTTTCGCTCAGCACTTCCCCCGTGTAGATCAGGTCGATGAGTCACTGCAA |
| | CCCCCACAGGCGACTGTGGCAGTGGCTGCGTTGGCGGCCTGCCTATGGGGA |
| | GACCCATAGGACGCTCTAATGTGGACATGGTGTGAAGAGCCTATTGAGCTA |
| | GTTAGTAGTCCTCCGGCCCCTGAATGCGGCTAATCCTAACTGCGGAGCACA |
| | CGCCCTCAACCCAGAGGGTAGTGTGTCGTAACGGGCAACTCTGCAGCGGAA |
| | CCGACTACTTTGGGTGTCCGTGTTTCTTATTATTCTTATATTGGCAGCTTATG |
| | GTGACAATTACAGAATTGTTACCATATAGCTATTGGATTGGCCATCCGGTGT |
| | GCAATAGAGCTGTTATATACCTGTTTGTTGGTTTTGTGCCACTAACTTTAAA |
| | ATCTATAGTCACTCTTAATTTCATATTGGCTCTCAACACAATTAAAC (SEQ |
| | ID NO: 232) |
| 4-36 | CGTGAATCTCGGCATCCCATCCATGCAACTTAGAAGTTTTTTAAACAAAGTT |
| | CAATAGAAGGGGGTACAAACCAGTGCCACCACGAACAAGCACTTCTGTTTC |
| | CCCGGTGAAGCTGCATAGACTGTCCCCACGGTTGAAAGCGGCAGATCCGTC |
| | ATCCGCTTTGGTACTTCGAGAAACCTAGTATCACCTTGGAATCTTCGATGCG |
| | TTGCGCTCAGCACTCAACCCCAGAGTGTAGCTTAGGTTGATGAGTCTGGAC |
| | AATCCTCACTGGCGACAGTGGTCCAGGCTGCGTTGGCGGCCTACCTGTGGT |
| | GAAAACCACAGGACGCTAGATGTGAACAAGGTGTGAAGAGTCTATTGAGC |
| | TACCAAAGAGTCCTCCGGCCCCTGAATGCGGCTAATCCTAACCACGGAGCA |
| | AGTACCCACAAACCAGTGGGCAGCTTGTCGTAACGCGCAAGTCTGTGGCGG |
| | AACCGACTACTTTGGGTGTCCGTGTTTCCTTTTTATTTTATCATGGCTGCTTA |
| | TGGTGACAATCTAAGATTGTTATCATATAGCTATTGGATTGGCCATCCGGTG |
| | ACTAACAGAGATATTATATACCTATTTGTTGGTTTTGTTAGATTAAATACTA |
| | TCACATTCAAAACTCTTCTTTATATCATACAATTGAACAGTAGAAAGAGAA |
| | A (SEQ ID NO: 233) |
| 4-37 | CGATCTTTGTCAAATACCGTTTAAAACAGCTCTGGGGTTGTACCCACCCCAG |
| | AGGCCCACGTGGCGGCTAGTACTCCGGTATTGCGGTACCCTTGTACGCCTG |
| | TTTTATACTCCCTTCCCGTAACTTAGACGCACAAAACCAAGTTCAATAGAA |
| | GGGGGTACAAACCAGTACCACCACGAACAAGCACTTCTGTTTCCCCGGTGA |
| | TGTCGTATAGACTGCTTGCGTGGTTGAAAGCGACGGATCCGTTATCCGCTTA |
| | TGTACTTCGAGAAGCCCAGTACCACCTCGGAATCTTCGATGCGTTGCGCTC |
| | AGCACTCAACCCCAGAGTGTAGCTTAGGCTGATGAGTCTGGACATCCCTCA |
| | CCGGTGACGGTGGTCCAGGCTGCGTTGGCGGCCTACCTATGGCTAACGCCA |
| | TGGGACGCTAGTTGTGAACAAGGTGTGAAGAGCCTATTGAGCTACATAAGA |
| | ATCCTCCGGCCCCTGAATGCGGCTAATCCCAACCTCGGAGCAGGTGGTCAC |
| | AAACCAGTGATTGGCCTGTCGTAACGCGCAAGTCCGTGGCGGAACCGACTA |
| | CTTTGGGTGTCCGTGTTTCCTTTTATTTTATTGTGGCTGCTTATGGTGACAAT |
| | CACAGATTGTTATCATAAAGCGAATTGGATTGGCCATCCGGTGAAAGTGAG |
| | ACTCATTATCTATCTGTTTGCTGGATCCGCTCCATTGAGTGTGTTTACTCTAA |
| | GTACAATTTCAACAGTTATTTCAATCAGACAATTGTATCATA (SEQ ID NO: |
| | 234) |
| 4-38 | CGATCTTTGTCAAATACCGTTTAAAACAGCTCTGGGGTTGTACCCACCCCAG |
| | AGGCCCACGTGGCGGCTAGTACTCCGGTATTGCGGTACCCTTGTACGCCTG |
| | TTTTATACTCCCTTCCCGTAACTTAGACGCACAAAACCAAGTTCAATAGAA |
| | GGGGGTACAAACCAGTACCACCACGAACAAGCACTTCTGTTTCCCCGGTGA |
| | TGTCGTATAGACTGCTTGCGTGGTTGAAAGCGACGGATCCGTTATCCGCTTA |
| | TGTACTTCGAGAAGCCCAGTACCACCTCGGAATCTTCGATGCGTTGCGCTC |
| | AGCACTCAACCCCAGAGTGTAGCTTAGGCTGATGAGTCTGGACATCCCTCA |
| | CCGGTGACGGTGGTCCAGGCTGCGTTGGCGGCCTACCTATGGCTAACGCCA |
| | TGGGACGCTAGTTGTGAACAAGGTGTGAAGAGCCTATTGAGCTACATAAGA |
| | ATCCTCCGGCCCCTGAATGCGGCTAATCCCAACCTCGGAGCAGGTGGTCAC |
| | AAACCAGTGATTGGCCTGTCGTAACGCGCAAGTCCGTGGCGGAACCGACTA |
| | CTTTGGGTGTCCGTGTTTCCTTTTATTTTATTGTGGCTGCTTATGGTGACAAT |
| | CACAGATTGTTATCATAAAGCGAATTGGATTGGCCATCCGGTGAAAGTGAG |
| | ACTCATTATCTATCTGTTTGCTGGATCCGCTCCATTGAGTGTGTTTACTCTAA |
| | GTACAATTTCAACAGTTATTTCAATCAGACAATTGTATCATA (SEQ ID NO: |
| | 234) |
| 4-39 | TTTAAAACAGCTCTAGGGTTGTTCCCACCCTAGAGGCCCAAGTGGCGGCTA |
| | GCACTCTGGTATTACGGTACCTTTGTGCGCCTGTTTTATATCCCTTCCCCCAT |
| | GTAACTTAGAAGATATTAAACAAAGTTCAATAGGAGGGGGTACAAACCAG |
| | TGCCACCACGAACAAACACTTCTGTTTCCCCGGTGAAGCTACATAGACTGT |
| | TCCCACGGTTGAAAGTGGCAGATCCGTTATCCGCTTTGGTACTTCGAGAAA |
| | CCTAGTACCACCTTGGAATCTTCGATGCGTTGCGCTCAGCACTCAACCCCAG |
| | AGTGTAGCTTAGGTCGATGAGTCTGGACGATCCTCACTGGCGACAGTGGTC |
| | CAGGCTGCGTTGGCGGCCTACCTGTGGCGAAAGCCACAGGACGCTAGTTGT |
| | GAACAAGGTGTGAAGAGTCTATTGAGCTACCAAAGAGTCCTCCGGCCCCTG |
| | AATGCGGCTAATCCCAACCACGGAGCAAGTGCCCACAAACCAGTGGGTGG |
| | CTTGTCGTAATGCGTAAGTCTGTGGCGGAACCGACTACTTTGGGTGTCCGTG |
| | TTTCCTTTTATTTTTAAACTGGCTGCTTATGGTGACAATCTAAGATTGTTATC |
| | ATATAGCTATTGGATTGGCCATCCGGTGACTAACAGAGATCTTGCATACCT |
| | GTTTGTTGGTTTTACTAAACTAGATATAGTTACATTTAAAACTCTTCTTTATA |
| | TCATACAGTTGAATAGTAGAAAGAGAAA (SEQ ID NO: 235) |
| 4-40 | TTTGCTCAGCGTAACTTCTCCGGGTTACGTGGAGACCAAAAGGCTACGGAG |
| | ACTCGGGCTACGGCCCTGGAGCACCTAGGTGCTCCTAAAGACGTTAGAAGT |
| | TGTACAAACTCGCCCAATAGGGCCCCCCAACCAGGGGGGTAGCGGGCAAG |
| | CACTTCTGTTTCCCCGGTATGATCTCATAGGCTGTACCCACGGCTGAAAGAG |
| | AGATTATCGTTACCCGCCTCACTACTTCGAGAAGCCCAGTAATGGTTCATG |
| | AAGTTGATCTCGTTGACCCGGTGTTTCCCCCACACCAGAAACCTGTGATGG |
| | GGGTGGTCATCCCGGTCATGGCGACATGACGGACCTCCCCGCGCCGGCACA |
| | GGGCCTCTTCGGAGGACGAGTGACATGGATTCAACCGTGAAGAGCCTATTG |
| | AGCTAGTGTTGATTCCTCCGCCCCCGTGAATGCGGCTAATCCCAACTCCGG |
| | AGCAGGCGGGCCCAAACCAGGGTCTGGCCTGTCGTAACGCGAAAGTCTGG |
| | AGCGGAACCGACTACTTTCGGGAAGGCGTGTTTCCTTTTGTTCCTTTTATCA |
| | AGTTTTATGGTGACAACTCCTGGTAGACGTTCCTGTGCGTCCTGTGAGAGAT |
| | TTCCAACAATTGAACAGACTAGAACCACTTGTTTTATCAAACCCTCACAGA |
| | ATAAGATAACA (SEQ ID NO: 236) |
| 4-41 | TTTGCTCAGCGTAACTTCTCCGGGTTACGTGGAGACCAAAAGGCTACGGAG |
| | ACTCGGGCTACGGCCCTGGAGCACCTAGGTGCTCCTAAAGACGTTAGAAGT |
| | TGTACAAACTCGCCCAATAGGGCCCCCCAACCAGGGGGGTAGCGGGCAAG |
| | CACTTCTGTTTCCCCGGTATGATCTCATAGGCTGTACCCACGGCTGAAAGAG |
| | AGATTATCGTTACCCGCCTCACTACTTCGAGAAGCCCAGTAATGGTTCATG |
| | AAGTTGATCTCGTTGACCCGGTGTTTCCCCCACACCAGAAACCTGTGATGG |
| | GGGTGGTCATCCCGGTCATGGCGACATGACGGACCTCCCCGCGCCGGCACA |
| | GGGCCTCTTCGGAGGACGAGTGACATGGATTCAACCGTGAAGAGCCTATTG |
| | AGCTAGTGTTGATTCCTCCGCCCCCGTGAATGCGGCTAATCCCAACTCCGG |
| | AGCAGGCGGGCCCAAACCAGGGTCTGGCCTGTCGTAACGCGAAAGTCTGG |
| | AGCGGAACCGACTACTTTCGGGAAGGCGTGTTTCCTTTTATTTTTAAACTGG |
| | CTTTTTATGGTGACAACTCCTGGTAGACGTTCCTGTGCGTCCTGTGAGAGAT |
| | TTCCAACAATTGAACAGACTAGAACCACTTGTTTTATCAAACCCTCACAGA |
| | ATAAGATAACA (SEQ ID NO: 237) |
| 4-42 | AGGAGTGCTGATATAGATTCGGATTTAGGCCGTGGTGCCCGTGGGCACGAC |
| | GGCCACGGCTCCAGCCGCACTCCTCTCCACGCCTTGTCCCACGGGACATAA |
| | AGTGGAGCCCTAAGCCCTAAACCCCCATGTTACCCAATTTTCTTATCCACTT |
| | CCCTTTTTCCCTTTGACTCACATTTTTCTTGACCGCGCTTGTGAGGCGGTTCC |
| | CGTACAGTTGAGTTGACGGGTCCCGCGCGGGACCTGAAAATTACTCCATAC |
| | TGATTAGGAAAGGTTCTAGTACGCTTGTACCCTTAAGGCCAGCGGGCTACC |
| | TCCTTTAGGTCTTATAGCCCCAACTGGTTAGAACCACTCCAGCTCACCTCTA |
| | TCGGTGCGGGAGTGCTACCACCCCTGCCTGCGTCAAGCCCGTTTTTCTAGTG |
| | ATCTGTCCACAAACACTTGGATTCCCGTCGCGACTGACAGAACCTGGCGAC |
| | AGGTGTCTCGATGTGTGTGACCAAGTGGCATGAGAATCGGAAACTAACGCT |
| | GGTAAGATGGTAAGTGCCCAATCCCTGTGGGGTGATGTTTTTCTCTGCATAG |
| | GAATGCTGCGAAGGTACCCCGGCCTTGAGTCGGGATCTGATCGCAGGCTAA |
| | TTGTCCTCTGTGCAGTTCATTTCCTTTTCTTTTCATTGGCATC (SEQ ID NO: |
| | 223) |
| 4-43 | TTTAAAACAGCTCTAGGGTTGTTCCCACCCTAGAGGCCCAAGTGGCGGCTA |
| | GCACTCTGGTATTACGGTACCTTTGTGCGCCTGTTTTATATCCCTTCCCCCAT |
| | GTAACTTAGAAGATATTAAACAAAGTTCAATAGGAGGGGGTACAAACCAG |
| | TGCCACCACGAACAAACACTTCTGTTTCCCCGGTGAAGCTACATAGACTGT |
| | TCCCACGGTTGAAAGTGGCAGATCCGTTATCCGCTTTGGTACTTCGAGAAA |
| | CCTAGTACCACCTTGGAATCTTCGATGCGTTGCGCTCAGCACTCAACCCCAG |
| | AGTGTAGCTTAGGTCGATGAGTCTGGACATTCCCCACTGGCGACAGTGGTC |
| | CAGGCTGCGTTGGCGGCCTACCTGTGGCGAAAGCCACAGGACGCTAGTTGT |
| | GAACAAGGTGTGAAGAGTCTATTGAGCTACCAAAGAGTCCTCCGGCCCCTG |
| | AATGCGGCTAATCCCAACCACGGAGCAAGTGCCCACAAACCAGTGGGTGG |
| | CTTGTCGTAATGCGTAAGTCTGTGGCGGAACCGACTACTTTGGGTGTCCGTG |
| | TTTCCTTTTATTTTTAAACTGGCTGCTTATGGTGACAATCTAAGATTGTTATC |
| | ATATAGCTATTGGATTGGCCATCCGGTGACTAACAGAGATCTTGCATACCT |
| | GTTTGTTGGTTTTACTAAACTAGATATAGTTACATTTAAAACTCTTCTTTATA |
| | TCATACAGTTGAATAGTAGAAAGAGAAA (SEQ ID NO: 238) |

Mutations of IRES and accessory sequences are encompassed herein to increase or reduce IRES activities, for example, by truncating the 5′ and/or 3′ ends of an IRES, adding a spacer 5′ to an IRES, modifying the 6 nucleotides 5′ to the translation initiation site (Kozak sequence), modification of (e.g., mutations) alternative translation initiation sites, and creating chimeric/hybrid IRES sequences. In some embodiments, the IRES sequence in the polynucleotide disclosed herein comprises one or more of these modifications relative to a natural or native IRES.

ii. Synthetic TIEs: Aptamer Complexes, Modified Nucleotides, IRES Variants & Other Engineered TIEs

In certain embodiments, a TIE provided herein is a synthetic TIE. In some embodiments, a synthetic TIE comprises aptamer complex, synthetic IRES, or other engineered TIE capable of initiating translation of a linear RNA or circular RNA polynucleotide.

In some embodiments, one or more aptamer sequences are capable of binding to a component of a eukaryotic initiation factor to either enhance or initiate translation. In some embodiments, an aptamer may be used to enhance translation in vivo and in vitro by promoting specific eukaryotic initiation factors (eIF) (e.g., certain aptamers disclosed in International Pat. Appl. No. PCT/EP2018/078794 are capable of binding to eukaryotic initiation factor 4F (eIF4F)). In some embodiments, an aptamer or a complex of aptamers may be capable of binding to EIF4G, EIF4E, EIF4A, EIF4B, EIF3, EIF2, EIF5, EIF1, EIF1A, 40S ribosome, PCBP1 (polyC binding protein), PCBP2, PCBP3, PCBP4, PABP1 (polyA binding protein), PTB, Argonaute protein family, HNRNPK (heterogeneous nuclear ribonucleoprotein K), or La protein.

b. Stop Codon or Stop Cassette

In various embodiments, the intervening region and/or core functional element comprises a stop codon or stop cassette. In some embodiments, the sequence is located downstream to a TIE and coding element. In some embodiments, the sequence is located downstream to a coding element and upstream to a TIE. In some embodiments, the intervening region comprises a stop codon. In one embodiment, the intervening region comprises a stop cassette. In some embodiments, the stop cassette comprises at least 2 stop codons. In some embodiments, the stop cassette comprises at least 2 frames of stop codons. In the same embodiment, the frames of the stop codons in a stop cassette each comprise 1, 2 or more stop codons. In some embodiments, the stop cassette comprises a LoxP or a RoxStopRox, or frt-flanked stop cassette. In the same embodiment, the stop cassette comprises a lox-stop-lox stop cassette.

### D. Additional Elements

In various embodiments, a provided polynucleotide (e.g., a DNA template, a linear precursor RNA polynucleotide, or a circular RNA polynucleotide) further comprises one or more elements for enhancing circularization, translation, or both. In certain embodiments, these elements are located with specificity between or within the intron elements, exon elements, or intervening region of the polynucleotide.

As an example, but not intended to be limiting, a polynucleotide, a precursor RNA polynucleotide, or circular RNA can comprise an IRES transacting factor region, a miRNA binding site, a restriction site, an RNA editing region, a structural or sequence element, a granule site, a zip code element, and/or an RNA trafficking element or another specialized sequence as found in the art that enhances promotes circularization and/or translation of the protein encoded within the circular RNA polynucleotide.

In some embodiments, the polynucleotide, precursor RNA polynucleotide, or circular RNA comprises an IRES transacting factor (ITAF) region. In some embodiments, the IRES transacting factor region modulates the initiation of translation through binding to PC-P1-PCBP4 (polyC binding protein), PABP1 (polyA binding protein), PTB (polyprimidine tract binding), Argonaute protein family, HNRNPK (Heterogeneous nuclear ribonucleoprotein K protein), or La protein. In some embodiments, the IRES transacting factor region comprises a polyA, polyC, polyAC, or polyprimidine track. In some embodiments, the ITAF region is located within the intervening region or core functional element. In some embodiments, the ITAF region is located within the TIE.

In certain embodiments, the polynucleotide, precursor RNA polynucleotide, or circular RNA comprises a lncRNA, miRNA, or a miRNA sponge. In certain embodiments, at least one miRNA binding site is included. In some embodiments the miRNA binding site is located within the 5′ intron element, 5′ exon element, intervening region or core functional element, 3′ exon element, and/or 3′ intron element. In some embodiments, the miRNA binding site is located within the spacer within the intron element or exon element. In certain embodiments, the miRNA binding site comprises the entire spacer regions. In some embodiments, the 5′ intron element and 3′ intron elements each comprise identical miRNA binding sites. In another embodiment, the miRNA binding site of the 5′ intron element comprises a different, in length or nucleotides, miRNA binding site than the 3′ intron element. In one embodiment, the 5′ exon element and 3′ exon element comprise identical miRNA binding sites. In other embodiments, the 5′ exon element and 3′ exon element comprise different, in length or nucleotides, miRNA binding sites. In some embodiments, the miRNA binding sites are located adjacent to each other within the circular RNA construct, linear mRNA construct, linear RNA polynucleotide precursor, and/or DNA template. In certain embodiments, the first nucleotide of one of the miRNA binding sites follows the first nucleotide last nucleotide of the second miRNA binding site. In some embodiments, the miRNA binding site is located within a translation initiation element (TIE) of an intervening region or core functional element. In one embodiment, the miRNA binding site is located before, trailing or within an internal ribosome entry site (IRES). In another embodiment, the miRNA binding site is located before, trailing, or within an aptamer complex.

Incorporation of miRNA sequences can permit tissue-specific expression of a coding sequence within an intervening region or core functional element. For example, in a circular RNA intended to express a protein in immune cells, miRNA binding sequences resulting in expression suppression in tissues such as the liver or kidney may be desired. Such miRNA binding sequences may be selected based on the cell or tissue expression of miRNAs. The unique sequences defined by the miRNA nomenclature are widely known and accessible to those working in the microRNA field. For example, they can be found in the miRDB public database. As a non-limiting example, one or more miR-122 target sites can be inserted in the circular RNA.

In some embodiments, the miR-122 site can comprise the following sequence:

(SEQ ID NO: 239)
CAAACACCATTGTCACACTCCAA

### E. Modifications

In certain embodiments, a provided polynucleotide (e.g., a precursor RNA polynucleotide, a circular RNA polynucleotide, or a DNA template) comprises modified nucleotides and/or modified nucleosides, namely comprising at least one modified A, C, G, or U/T nucleotide or nucleoside. As exhibited by the exemplary nucleotide or nucleotide modification presented below, such modifications differ from mutations selected from insertions, deletions, addition, or subtraction of nucleotides, for example, the mutations in a permuted Group I and Group II intron segment.

In some embodiments, the polynucleotide is a precursor RNA polynucleotide and comprises at least one modified A, C, G, or U nucleotide or nucleoside. In some embodiments, the precursor RNA polynucleotide is linear. In some embodiments, the precursor RNA polynucleotide is capable of producing a circular RNA comprising at least one modified nucleotide or nucleoside after splicing. In some embodiments, the precursor RNA polynucleotide comprising one or more modified nucleotide or nucleoside is capable of circularizing when incubated in the presence of one or more guanosine nucleotides or nucleoside (e.g., GTP) and a divalent cation (e.g., Mg2+). In some embodiments, the polynucleotide is a circular RNA polynucleotide and comprises at least one modified A, C, G, or U nucleotide or nucleoside modifications.

In some embodiments, modified nucleotides or nucleosides occur throughout a precursor RNA polynucleotide. In some embodiments, the RNA polynucleotide comprises 5′ and 3′ combined accessory elements comprising one or more modified nucleotides. In some embodiments, the RNA polynucleotide comprises an intron element and/or exon element comprising one or more modified nucleotide or nucleoside.

In some embodiments, portions of the 3′ and/or 5′ intron and/or exon segments in a linear precursor RNA polynucleotide of the present disclosure contain modified nucleotides or nucleosides. In some embodiments, the secondary structures of at least the intron and/or exon segments are preserved. In some embodiments, the terminal element comprises at least one modified nucleotide or nucleoside. In some embodiments, the terminal element, intervening region, and/or monotron comprises at least one modified nucleotide or nucleoside. In certain embodiments, the RNA polynucleotide comprises a spacer comprising at least one modified nucleotide or nucleoside. In certain embodiments, the RNA polynucleotide comprises a duplex comprising at least one modified nucleotide or nucleoside. In certain embodiments, the RNA polynucleotide comprises an affinity sequence comprising at least one modified nucleotide or nucleoside. In certain embodiments, the RNA polynucleotide comprises a leading and/or lagging strand comprising at least one modified nucleotide or nucleoside. In some embodiments, the RNA polynucleotide comprises an expression sequence encoding a CD19 binder comprising at least one modified nucleotide or nucleoside. In some embodiments, the RNA polynucleotide comprises a translation initiation element (TIE) comprising at least one modified nucleotide or nucleoside. In certain embodiments, the polynucleotide comprises a stop codon and/or stop cassette comprising one or more modified nucleotide or nucleoside.

In some embodiments, a precursor RNA polynucleotide comprising at least one modified A, C, G, or U nucleotide or nucleoside comprises at least a portion of each of:
a. a 5′ combined accessory element, comprising:
   i. a 3′ intron segment,
   ii. a 3′ exon segment,
b. an intervening region comprising an internal ribosome entry site (IRES) and an expression sequence encoding a CD19 binding molecule,
c. a 3′ combined accessory element, comprising:
   i. a 5′ exon segment, and
   ii. a 5′ intron segment.

In some embodiments, a circular RNA comprising at least one modified A, C, G, or U nucleotide or nucleoside comprises at least a portion of each of:
a. a post-splicing 3′ exon segment,
b. optionally a 5′ internal homology region,
c. optionally a 5′ spacer,
d. an intervening region comprising an internal ribosome entry site (IRES) and an expression sequence encoding a CD19 binding molecule,
e. optionally a 3′ spacer,
f. optionally a 3′ internal homology region, and
g. a post-splicing 5′ exon segment.

In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i⁶A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² 2G (N²,N²-dimethylguanosine); m²Gm (N²,2′-O-dimethylguanosine); m²₂Gm (N²,N²,2′-O-trimethylguanosine); Gr(p) (2′-O-ribosylguanosine(phosphate)); yW (wybutosine); o₂yW (peroxywybutosine); oHyW (hydroxywybutosine); OhyW* (undermodified hydroxy wybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵U^{m}(5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶^{²}A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ 2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); N1-methylpseudouridine; or ac⁶A (N⁶-acetyladenosine).

In some embodiments, the modified nucleoside may include a compound selected from the group of: 170yridine-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-methylpseudouridine; and N2,N2-dimethyl-6-thio-guanosine.

In another embodiment, the modifications are independently selected from 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine.

In some embodiments, the modified nucleoside is N1-methylpseudouridine.

In some embodiments, the modified nucleotide or nucleoside is selected from one or more of: 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine, 5-(2-amino)propyl uridine, 5-halocytidine, 5-halouridine, 4-acetylcytidine, 1-methyladenosine, 2-methyladenosine, 3-methyicytidine, 6-methyluridine, 2-methylguanosine, 7-methylguanosine, 2,2-dimethylguanosine, 5-methylaminoethyluridine, 5-methyloxyuridine, 7-deaza-adenosine, 6-azouridine, 6-azocytidine, 6-azothymidine, 5-methyl-2-thiouridine, 2-thiouridine, 4-thiouridine, 2-thiocytidine, dihydrouridine, pseudouridine, queuosine, archaeosine, naphthyl substituted naphthyl groups, an O- and N-alkylated purines and pyrimidines, N6-methyladenosine, 5-methylcarbonylmethyluridine, uridine 5-oxyacetic acid, pyridine-4-one, pyridine-2-one, aminophenol, 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

Additional modified nucleotides and nucleosides can be selected from clinically validated modified nucleotides described in the art. See, e.g., US20190345503A1 (m⁶A-modified circRNA); US20220288176A1 (m⁶A modification of circRNA); US20220251578A1 (at least one N6-methyladenosine (m⁶A)); WO2022271965A2 (N6-methyladenosine, 2-thiouridine, and 2′-O-methylcytidine), which are each incorporated by reference in their entireties.

In some embodiments, a first and second precursor polynucleotide are provided, where the first precursor RNA polynucleotide comprises a 3′ intron fragment of a first intron (Intron 1), a 5′ intron fragment of a second intron (Intron 2), a translation initiation element, a fragment of a sequence of interest (e.g., coding region encoding a CD19 binding molecule), and two exon fragments that correspond with the intron fragments; and the second precursor comprises a 3′ intron fragment of the second intron (Intron 2) and a 5′ intron fragment of the first intron (Intron 1), a fragment of the sequence of interest of the first precursor, and exon fragments corresponding to those in the first precursor. In these embodiments, the first and second linear precursor RNA polynucleotides are capable of forming a circular RNA. In some embodiments, the first precursor comprises no nucleotide or nucleoside modifications and the second precursor comprises nucleotide or nucleoside modifications. In some embodiments, the first precursor comprises nucleotide or nucleoside modifications and the second precursor comprises no nucleotide or nucleoside modifications. In some embodiments, the first precursor and the second precursor comprise no nucleotide or nucleoside modifications. In some embodiments, the first precursor and the second precursor comprise nucleotide or nucleoside modifications.

Indeed, contrary to publications contending that, for example, “[i]ncorporation of m6A modification into circRNA does not affect splicing to form circRNA” (see, e.g., Chen et al., 2019, Mol Cell, N6-Methyladenosine Modification Controls Circular RNA Immunity), the disclosures herein demonstrate that the incorporation of certain nucleotide and/or nucleoside modifications to a precursor RNA polynucleotide can affect the circularization and/or splicing of the circular RNA. (See Kariko et al., 2005, Immunity, Suppression of RNA recognition by Toll-like receptors: the impact of nucleoside modification and the evolutionary origin of RNA; Kariko et al., 2005, Mol Ther, Incorporation of pseudouridine into mRNA yields superior nonimmunogenic vector with increased translational capacity and biological stability; Wesselhoeft et al., 2019, Mol Cell, RNA Circularization Diminishes Immunogenicity and Can Extend Translation Duration In Vivo; Chen et al., 2022, Nature Biotechnology, Engineering circular RNA for enhanced protein production). Modified nucleotide or nucleosides may exhibit different physical properties to their unmodified counterparts. In some embodiments, the presence of a modified nucleotide or nucleoside can affect the folding patterns and/or function of an accessory element, translation initiation element (TIE), and/or an expression sequence encoding a CD19 binding molecule within the circular RNA or linear precursor. Position and composition of a nucleotide or nucleoside modification in a polynucleotide are impacted by the nucleotide or nucleoside composition (i.e., A, C, G, or U nucleotide or nucleoside) of the accessory elements, TIE, or expression sequence(s) encoding a CD19 binding molecule.

In some embodiments, in a provided polynucleotide (e.g., a precursor RNA polynucleotide, or a circular RNA polynucleotide, described in more detail elsewhere herein), between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are unmodified. In some embodiments, a provided polynucleotide (e.g., a precursor RNA polynucleotide, or a circular RNA polynucleotide, described in more detail elsewhere herein) comprises modified nucleotides and/or modified nucleosides where between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. In some embodiments, between 1% and 10% of the nucleotides or nucleosides are modified.

In some embodiments, in portions of the polynucleotide (e.g., a precursor RNA polynucleotide, or a circular RNA polynucleotide, described in more detail elsewhere herein), between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides are modified. For example, in some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the intervening region are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the IRES are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the noncoding or coding region are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ intron segment and/or 3′ intron segment are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ exon segment or post-splicing exon segment and/or 3′ exon segment or post-splicing exon segment are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ spacer and/or 3′ spacer are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90%, or 90% and 100% of the nucleotides or nucleosides in the 5′ homology region and/or 3′ homology region are modified. In some embodiments, the secondary structures of at least the intron and/or exon segments are preserved. In some embodiments, the secondary structure of the coding or noncoding region is preserved. In some embodiments, the IRES is unmodified or substantially unmodified to preserve secondary structure needed to initiate translation.

In some embodiments, between 1% and 10% of the nucleotides or nucleosides are modified in a polynucleotide of the present disclosure (e.g., a precursor RNA polynucleotide, or a circular RNA polynucleotide, described in more detail elsewhere herein). In some embodiments, portions of the polynucleotide comprise between 1% and 10% modification of the nucleotides or nucleosides. For example, in some embodiments, between 1% and 10% of the nucleotides or nucleosides in the intervening region are modified. In some embodiments, between 11% and 10% of the nucleotides or nucleosides in the IRES are modified. In some embodiments, between 1% and 10% of the nucleotides or nucleosides in the noncoding or coding region are modified. In some embodiments, between 1% and 10% of the nucleotides or nucleosides in the 5′ intron segment and/or 3′ intron segment are modified. In some embodiments, between 11% and 10% of the nucleotides or nucleosides in the 5′ exon segment or post-splicing exon segment and/or 3′ exon segment or post-splicing exon segment are modified. In some embodiments, between 1% and 10% of the nucleotides or nucleosides in the 5′ spacer and/or 3′ spacer are modified. In some embodiments, between 1% and 10% of the nucleotides or nucleosides in the 5′ homology region and/or 3′ homology region are modified.

In some embodiments, the polynucleotides comprising modified nucleotides and/or modified nucleosides provide additional stability and resistance to immune activation. In some embodiments, polynucleotides comprising modified nucleotides and/or modified nucleosides maintain stability and resistance to immune activation as compared to a corresponding polynucleotide comprising no modified nucleotides and/or modified nucleosides.

In some embodiments, a precursor RNA polynucleotide with modified nucleotides and/or nucleosides improves circularization as compared to a corresponding linear precursor RNA polynucleotide comprising no nucleotide or nucleoside modifications or other appropriate control. In other embodiments, a precursor RNA polynucleotide with modified nucleotides and/or nucleosides maintains the same circularization as compared to a corresponding precursor RNA polynucleotide comprising no nucleotide or nucleoside modifications or other appropriate control. In some embodiments, the precursor polynucleotides comprising modified nucleotides and/or modified nucleosides maintain circularization at greater than 50%, greater than 60%, greater than 70%, greater than 80%, greater than 90%, or greater than 100% (i.e., improves circularization), as compared to a corresponding precursor polynucleotide comprising no nucleotide or nucleoside modifications. In some embodiments, the precursor polynucleotides maintain circularization at greater than 70% as compared to a corresponding precursor polynucleotide comprising no nucleotide or nucleoside modifications. In some embodiments, the precursor polynucleotides maintain circularization at greater than 80% as compared to a corresponding precursor polynucleotide comprising no nucleotide or nucleoside modifications. In some embodiments, the precursor polynucleotides maintain circularization at greater than 90% as compared to a corresponding precursor polynucleotide comprising no nucleotide or nucleoside modifications. In some embodiments, the precursor polynucleotides exhibit greater than 100% circularization (i.e., improved circularization) as compared to a corresponding precursor polynucleotide comprising no nucleotide or nucleoside modifications.

In some embodiments a circular RNA with modified nucleotides and/or nucleosides reduces immunogenicity and/or improves translation of the coding region as compared to a corresponding circular RNA comprising no nucleotide or nucleoside modifications. In other embodiments, a circular RNA polynucleotide with modified nucleotides and/or nucleosides maintains the same immunogenicity and/or translation of the coding region as compared to a corresponding circular RNA comprising no nucleotide or nucleoside modifications. For example, in some embodiments, the circular RNAs described herein comprising at least one modified A, C, G, or U nucleotide or nucleoside exhibit reduced immunogenicity, without losing circularization and/or translation. In some embodiments, the circular RNAs described herein exhibit immunogenicity that is reduced by about 10% to about 99%, for example reduced by about 10%, 15%, 20%, 25%, 30%, 35%, 40%, 45%, 50%, 55%, 60%, 65%, 70%, 75%, 80%, 85%, 90%, 95%, or 99% as compared to a corresponding circular RNA comprising no nucleotide or nucleoside modifications. In some embodiments, the polynucleotides comprising modified nucleosides provide additional stability and resistance to immune activation.

In some embodiments, portions of the polynucleotide (e.g., a precursor RNA polynucleotide, or a circular RNA polynucleotide) or the polynucleotide in its entirety comprises no nucleotide or nucleoside modifications. In some embodiments, portions of the polynucleotide (e.g., a precursor RNA polynucleotide, or a circular RNA polynucleotide, described in more detail elsewhere herein) comprise less than 10% modification. For example, in some embodiments, the intervening region comprises no nucleotide or nucleoside modifications or less than 10% of the nucleotides or nucleosides therein are modified. In some embodiments, the IRES comprises no nucleotide or nucleoside modifications or less than 10% of the nucleotides or nucleosides therein are modified. In some embodiments, the noncoding or coding region comprises no nucleotide or nucleoside modifications or less than 10% of the nucleotides or nucleosides therein are modified. In some embodiments, the 5′ intron segment and/or 3′ intron segment comprises no nucleotide or nucleoside modifications or less than 10% of the nucleotides or nucleosides therein are modified. In some embodiments, the 5′ exon segment or post-splicing exon segment and/or 3′ exon segment or post-splicing exon segment comprises no nucleotide or nucleoside modifications or less than 10% of the nucleotides or nucleosides therein are modified. In some embodiments, the 5′ spacer and/or 3′ spacer comprises no nucleotide or nucleoside modifications or less than 10% of the nucleotides or nucleosides therein are modified. In some embodiments, the 5′ homology region, 3′ homology region comprises no nucleotide or nucleoside modifications or less than 10% of the nucleotides or nucleosides therein are modified.

In some embodiments herein, between 1% and 10% of the nucleotides or nucleosides are modified in a linear precursor RNA polynucleotide or circular RNA of the present disclosure. In some embodiments in a linear precursor RNA polynucleotide or circular RNA of the present disclosure, the intervening region comprises no nucleotide or nucleoside modifications or is less than 10% modified; the IRES comprises no nucleotide or nucleoside modifications or is less than 10% modified; the noncoding or coding region comprises no nucleotide or nucleoside modifications or is less than 10% modified; the 5′ intron segment and/or 3′ intron segment comprises no nucleotide or nucleoside modifications or is less than 10% modified; the 5′ exon segment or post-splicing exon segment and/or 3′ exon segment or post-splicing exon segment comprises no nucleotide or nucleoside modifications or is less than 10% modified; the 5′ spacer and/or 3′ spacer comprises no nucleotide or nucleoside modifications or is less than 10% modified; and/or the 5′ homology region, 3′ homology region comprises no nucleotide or nucleoside modifications or is less than 10% modified.

In some embodiments, modified nucleotides or nucleotides occur throughout a precursor RNA polynucleotide. In other embodiments, portions of the 3′ and/or 5′ intron and/or exon segments in a linear precursor RNA polynucleotide of the present disclosure contain modified nucleotides or nucleosides, but the remaining portions of the linear precursor do not comprise nucleotide or nucleoside modifications. In some embodiments, portions of the 3′ and/or 5′ intron and/or exon segments in a linear precursor RNA polynucleotide of the present disclosure contain modified nucleotides or nucleosides, but the remaining portions of the linear precursor comprise minimal nucleotide or nucleoside modifications. In some embodiments, portions of the 3′ and/or 5′ intron and/or exon segments in a linear precursor RNA polynucleotide of the present disclosure contain modified nucleotides or nucleosides, but the remaining portions of the linear precursor comprise less than 90%, 80%, 70%, 60%, 50%, 40%, 30%, 20%, or 10% modified nucleotides or nucleosides.

In some embodiments, where the circular RNA is produced from a linear precursor and where the linear precursor is modified at the 3′ and/or 5′ ends only, the circular RNA contains only the modified nucleotide or nucleosides that remain after circularization.

In certain embodiments, a circular RNA is prepared by providing modified nucleotides or nucleosides to precursor RNA comprising:
a. a 5′ combined accessory element, comprising:
   i. a 3′ intron segment,
   ii. a 3′ exon segment,
b. an intervening region comprising an internal ribosome entry site (IRES) and a noncoding or coding region,
c. a 3′ combined accessory element, comprising:
   i. a 5′ exon segment, and
   ii. a 5′ intron segment.

In certain embodiments, a circular RNA is prepared by providing a first and second linear precursor RNA polynucleotide, wherein the first and second linear precursor RNA polynucleotides are capable of forming a circular RNA. In some embodiments, either the first precursor or the second precursor but not both precursors comprises at least one modified A, C, G, or U nucleotide or nucleoside. In some embodiments, the first precursor comprises at least one modified A, C, G, or U nucleotide or nucleoside and the second precursor comprises no modified nucleotides or nucleosides. In some embodiments, the second precursor comprises least one modified A, C, G, or U nucleotide or nucleoside and the first precursor comprises no modified nucleotides or nucleosides.

In some embodiments, the first precursor comprises a 3′ intron fragment of a first intron (Intron 1), a 5′ intron fragment of a second intron (Intron 2), a translation initiation element, a fragment of a sequence of interest (e.g., coding region), and two exon fragments that correspond with the intron fragments. In some embodiments, the second precursor comprises a 3′ intron fragment of the second intron (Intron 2) and a 5′ intron fragment of the first intron (Intron 1), a fragment of the sequence of interest of the first precursor, and exon fragments corresponding to those in the first precursor.

In some embodiments, the TIE of the first precursor RNA polynucleotides comprises an IRES. In some embodiments, the first precursor RNA polynucleotide comprises a noncoding or coding region.

In some embodiments, the first and second precursor RNA polynucleotides further comprise spacers and/or homology arms.

In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the first linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the first linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the first linear precursor are unmodified.

In some embodiments, in portions of the first linear polynucleotide, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides are modified. For example, in some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the intervening region of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the TIE (e.g., IRES) of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the noncoding or coding region of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the 5′ and/or 3′ intron fragment of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the 5′ and/or 3′ exon fragment of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the spacer of the first linear polynucleotide are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the internal and/or external homology region of the first linear polynucleotide are modified.

In some embodiments, in portions of the first linear polynucleotide, less than 10% of the nucleotides or nucleosides are modified. For example, in some embodiments, less than 10% of the nucleotides or nucleosides in the intervening region of the first linear polynucleotide are modified. In some embodiments, less than 10% of the nucleotides or nucleosides in the TIE (e.g., IRES) of the first linear polynucleotide are modified. In some embodiments, less than 10% of the nucleotides or nucleosides in the noncoding or coding region of the first linear polynucleotide are modified. In some embodiments, less than 10% of the nucleotides or nucleosides in the 5′ and/or 3′ intron fragment of the first linear polynucleotide are modified. In some embodiments, less than 10% of the nucleotides or nucleosides in the 5′ and/or 3′ exon fragment of the first linear polynucleotide are modified. In some embodiments, less than 10% of the nucleotides or nucleosides in the spacer of the first linear polynucleotide are modified. In some embodiments, less than 10% of the nucleotides or nucleosides in the internal and/or external homology region of the first linear polynucleotide are modified.

In some embodiments, the intervening region of the first linear polynucleotide comprises no nucleotide or nucleoside modifications. In some embodiments, the TIE (e.g, IRES) of the first linear polynucleotide comprises no nucleotide or nucleoside modifications. In some embodiments, the noncoding or coding region of the first linear polynucleotide comprises no nucleotide or nucleoside modifications. In some embodiments, the 5′ and/or 3′ intron fragment of the first linear polynucleotide comprises no nucleotide or nucleoside modifications. In some embodiments, the 5′ and/or 3′ exon fragment of the first linear polynucleotide comprises no nucleotide or nucleoside modifications. In some embodiments, the spacer of the first linear polynucleotide comprises no nucleotide or nucleoside modifications. In some embodiments, the internal and/or external homology region of the first linear polynucleotide comprises no nucleotide or nucleoside modifications.

In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the second linear precursor are modified. In some embodiments, between 0% and 99%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 99% of the nucleotides or nucleosides in the second linear precursor are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the second linear precursor are unmodified.

In some embodiments, in portions of the second linear polynucleotide, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the noncoding or coding region of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the 5′ and/or 3′ intron fragment of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the 5′ and/or 3′ exon fragment of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the spacer of the second linear polynucleotide are modified. In some embodiments, between 1% and 100%, 1% and 2%, 1% and 3%, 1% and 4%, 1% and 5%, 5% and 6%, 5% and 7%, 5% and 8%, 5% and 9%, 5% and 10%, 10% and 20%, 20% and 30%, 30% and 40%, 40% and 50%, 50% and 60%, 60% and 70%, 70% and 80%, 80% and 90% or 90% and 100% of the nucleotides or nucleosides in the internal and/or external homology region of the second linear polynucleotide are modified.

In some embodiments, in portions of the second linear polynucleotide, less than 10% of the nucleotides or nucleosides are modified. For example, in some embodiments, less than 10% of the nucleotides or nucleosides in the noncoding or coding region of the second linear polynucleotide are modified. In some embodiments, less than 10% of the nucleotides or nucleosides in the 5′ and/or 3′ intron fragment of the second linear polynucleotide are modified. In some embodiments, less than 10% of the nucleotides or nucleosides in the 5′ and/or 3′ exon fragment of the second linear polynucleotide is modified. In some embodiments, less than 10% of the nucleotides or nucleosides in the spacer of the second linear polynucleotide are modified. In some embodiments, less than 10% of the nucleotides or nucleosides in the internal and/or external homology region of the second linear polynucleotide are modified.

For example, in some embodiments, the noncoding or coding region of the second linear polynucleotide comprises no nucleotide or nucleoside modifications. In some embodiments, the 5′ intron fragment and/or 3′ intron fragment of the second linear polynucleotide comprises no nucleotide or nucleoside modifications. In some embodiments, the 5′ exon fragment and/or 3′ exon fragment of the second linear polynucleotide comprises no nucleotide or nucleoside modifications. In some embodiments, the spacer of the second linear polynucleotide comprises no nucleotide or nucleoside modifications. In some embodiments, the internal and/or external homology region of the second linear polynucleotide comprises no nucleotide or nucleoside modifications.

In some embodiments, in a first linear precursor of the present disclosure, the intervening region comprises no nucleotide or nucleoside modifications or is less than 10% modified; the TIE (e.g., IRES) comprises no nucleotide or nucleoside modifications or is less than 10% modified; the noncoding or coding region comprises no nucleotide or nucleoside modifications or is less than 10% modified; the 5′ and/or 3′ intron fragment comprises no nucleotide or nucleoside modifications or is less than 10% modified; the 5′ and/or 3′ exon fragment comprises no nucleotide or nucleoside modifications or is less than 10% modified; the spacer comprises no nucleotide or nucleoside modifications or is less than 10% modified; and/or the internal and/or external homology region comprises no nucleotide or nucleoside modifications or is less than 10% modified.

In some embodiments, in a second linear precursor of the present disclosure, the noncoding or coding region comprises no nucleotide or nucleoside modifications or is less than 10% modified; the 5′ intron fragment and/or 3′ intron fragment comprises no nucleotide or nucleoside modifications or is less than 10% modified; the 5′ exon fragment and/or 3′ exon fragment comprises no nucleotide or nucleoside modifications or is less than 10% modified; the spacer comprises no nucleotide or nucleoside modifications or is less than 10% modified; and/or the internal and/or external homology region comprises no nucleotide or nucleoside modifications or is less than 10% modified.

In some embodiments, incorporation of a nucleotide or nucleoside modification to a precursor RNA polynucleotide hinders or lowers the capacity of the circular RNA to circularize, splice, or express. In some embodiments, the precursor polynucleotide comprising no modified nucleotides and/or nucleosides maintains or improves circularization as compared to a precursor polynucleotide comprising one or more nucleotide or nucleoside modification. In some embodiments, the precursor polynucleotide comprising no modified nucleotides or nucleosides maintains circularization at greater than 50%, greater than 60%, greater than 70%, greater than 80%, greater than 90%, or greater than 100% (i.e., improves circularization), as compared to a corresponding precursor polynucleotide comprising one or more nucleotide or nucleoside modification.

In some embodiments, the polynucleotides comprising no nucleotide or nucleoside modifications, for an example a circular RNA, has comparable or reduced immunogenicity as compared to a polynucleotide comprising one or more nucleotide or nucleoside modification. In some embodiments, the circular RNAs described herein (i.e., a circular RNA polynucleotide comprising no nucleotide or nucleoside modification) exhibit immunogenicity that is reduced by about 10% to about 99%, for example reduced by about 10%, 15%, 20%, 25%, 30%, 35%, 40%, 45%, 50%, 55%, 60%, 65%, 70%, 75%, 80%, 85%, 90%, 95%, or 99% as compared to a corresponding circular RNA comprising one or more nucleotide or nucleoside modifications. In some embodiments, the polynucleotides comprising no modified nucleotides and/or modified nucleosides, for example a circular RNA, maintain or improve translation of a coding region as compared to a corresponding polynucleotide comprising one or more nucleotide or nucleoside modifications. In some embodiments, the polynucleotides comprising no modified nucleosides provide additional stability and resistance to immune activation. In some embodiments, for example, the polynucleotide comprising no modified A, C, G, or U nucleotide or nucleoside maintains expression at greater than 50%, greater than 60%, greater than 70%, greater than 80%, greater than 90%, or greater than 100%, as compared to a corresponding precursor polynucleotide comprising one or more nucleotide or nucleoside modifications. In some embodiments, the non-modified polynucleotides maintain expression at greater than 70% as compared to a corresponding polynucleotide comprising one or more nucleotide or nucleoside modifications. In some embodiments, the non-modified polynucleotides maintain expression at greater than 80% as compared to a corresponding polynucleotide comprising one or more nucleotide or nucleoside modifications. In some embodiments, the non-modified polynucleotides maintain expression at greater than 90% as compared to a corresponding polynucleotide comprising one or more nucleotide or nucleoside modifications. In some embodiments, the non-modified polynucleotides exhibit greater than 100% expression (i.e., improved expression) as compared to a corresponding polynucleotide comprising one or more nucleotide or nucleoside modifications. In some embodiments, the non-modified polynucleotides exhibit greater purification efficacy as compared to a corresponding polynucleotide comprising one or more nucleotide or nucleoside modifications. In some embodiments, for example, the polynucleotide comprising no modified A, C, G, or U nucleotide or nucleoside exhibits greater purification efficacy at greater than 50%, greater than 60%, greater than 70%, greater than 80%, greater than 90%, or greater than 100%, as compared to a corresponding precursor comprising one or more nucleotide or nucleoside modification.

### F. Codon Optimization

In particular embodiments, polynucleotides may be codon-optimized. A codon-optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the core functional element. In some embodiments, a codon-optimized sequence comprises a codon-optimized CAR sequence. In some embodiments, a codon-optimized sequence comprises a codon-optimized antigen binding domain sequence. Codon optimization can be performed by methods known in the art.

### 3. Expression Sequences and Payloads

In various embodiments, a provided polynucleotide (e.g., a DNA template, a linear RNA polynucleotide, or a circular RNA polynucleotide) comprises at least one expression sequence encoding a CD19 binding molecule, e.g., CD19 CAR. In certain embodiments, the polynucleotide comprises the expression sequence and a TIE. In certain embodiments, the polynucleotide comprises the expression sequence and an IRES, wherein the IRES can facilitate expression of the protein when delivered in vivo. In some embodiments, the expression sequence is a part of the intervening region or core functional element located in between the 5′ end and 3′ end of a linear precursor RNA polynucleotide or resultant circular RNA.

In some embodiments, a provided polynucleotide (e.g., a DNA template, a linear RNA polynucleotide, or a circular RNA polynucleotide) encodes one or more chimeric antigen receptors (CARs). CARs are genetically-engineered receptors. These engineered receptors may be inserted into and expressed by immune cells, including T cells via circular RNA as described herein. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. In some embodiments, the CAR encoded by the polynucleotide comprises (i) an antigen-binding molecule that specifically binds to a target antigen, (ii) a hinge domain, a transmembrane domain, and an intracellular domain, and (iii) an activating domain.

In some embodiments, an orientation of the CARs in accordance with the disclosure comprises an antigen binding domain (such as an scFv or VHH) in tandem with a costimulatory domain and an activating domain. The costimulatory domain may comprise one or more of an extracellular portion, a transmembrane portion, and an intracellular portion. In other embodiments, multiple costimulatory domains may be utilized in tandem.

i. Antigen Binding Domain

CARs may be engineered to bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (scFv). An scFv is a single chain antibody fragment having the variable regions of the heavy and light chains of an antibody linked together. See U.S. Pat. Nos. 7,741,465, and 6,319,494, as well as Eshhar et al., Cancer Immunol Immunotherapy (1997) 45: 131-136. An scFv retains the parent antibody's ability to specifically interact with target antigen. scFvs are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest (e.g., a CD19 molecule). Bispecific and multispecific CARs are contemplated within the scope of the disclosure, with specificity to more than one target of interest.

In some embodiments, the antigen binding molecule comprises a single chain, wherein the heavy chain variable region and the light chain variable region are connected by a linker. In some embodiments, the VH is located at the N terminus of the linker and the VL is located at the C terminus of the linker. In other embodiments, the VL is located at the N terminus of the linker and the VH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids.

In some embodiments, the antigen binding molecule comprises a single domain antibody (sdAB). The sdAB is a single chain antibody, wherein the N-terminal of the domain is capable of binding to an antigen without requiring domain pairing. See Harmsen & Haard, Appl. Microbiol. Biotechnol., Volume 77, No. 1, 2007 (13-22); see also Wilton et al., ACS, Synth. Biol, Volume 7, No. 11, 2018 (2480-2484). In some embodiments, the sdAB is a heavy chain antibody (hcAbs). In some embodiments, the heavy chain antibody comprises only heavy chains. In some embodiments, the antigen binding molecule (e.g., the heavy chain antibody) comprises a nanobody or variable heavy domain of heavy chain (VHH) sequence, or a variable domain of a new antigen receptor (VNAR) sequence. In some embodiments, the antigen binding molecule (e.g., the heavy chain antibody) may bind to an antigen without domain pairing using three complementarity-determining regions (CDRs)). In some embodiments, embodiments, the antigen biding molecule (e.g., the heavy chain antibody) comprises one, two or three CDRs. In some embodiments, the sdAB comprises a camelid sdAb.

In some embodiments, the antigen binding molecule comprises a VHH sequence. In some embodiments, the VHH sequence lacks a CH1 domain. In some embodiments, the VHH sequence comprises a framework region (FR). In some embodiments, the VHH sequence comprises one, two, three and/or four framework regions. In some embodiments, the VHH sequence comprises one, two or three CDRs.

In certain embodiments, the CAR comprises one, two, or more VHH sequences. In certain embodiments, the CAR comprises two VHH sequences bound by a linker sequence (e.g., a G4S linker sequence). In some embodiments, the first VHH is located at the N terminus of the linker and the second VHH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids. In some embodiments, the linker sequence comprises a G4S linker sequence (i.e., glycine-glycine-glycine-glycine-serine sequence). In some embodiments, the linker sequence comprises at least one G4S linker sequence. In some embodiments, the linker sequence comprises one, two, three, four, or five G4S linker sequences. In some embodiments, the linker sequence comprises more than five G4S linker sequences. In some embodiments, the G4S linker sequence comprises the sequence of SEQ ID NO: 1.

In certain embodiments, a circular RNA and/or linear mRNA encoding a CAR comprising a VHH sequence comprises greater or comparable levels of CAR expression in an immune cell compared to circular RNA and/or linear mRNA encoding a CAR comprising a scFv or an alternative antigen binding domain available in the art. In certain embodiments, a circular RNA and/or linear mRNA encoding a CAR comprising a VHH sequence comprises greater or comparable cytotoxicity levels in an immune cell compared to a circular RNA and/or linear mRNA encoding a CAR comprising a scFv or an alterative antigen binding domain available in the art.

In some embodiments, the antigen binding molecule comprises a DARPin. In some embodiments, the antigen binding molecule comprises an anticalin or other synthetic protein capable of specific binding to target protein.

In some embodiments, the CAR comprises an antigen binding domain specific for an antigen selected from the group CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-IIRa), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), MAGE family members (including MAGE-A1, MAGE-A3 and MAGE-A4), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLECI2A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), MUC16, 5T4, 8H9, avP0 integrin, avP6 integrin, alphafetoprotein (AFP), B7-H6, ca-125, CA9, CD44, CD44v7/8, CD52, E-cadherin, EMA (epithelial membrane antigen), epithelial glycoprotein-2 (EGP-2), epithelial glycoprotein-40 (EGP-40), ErbB4, epithelial tumor antigen (ETA), folate binding protein (FBP), kinase insert domain receptor (KDR), k-light chain, L1 cell adhesion molecule, MUC18, NKG2D, oncofetal antigen (h5T4), tumor/testis-antigen 1B, GAGE, GAGE-1, BAGE, SCP-1, CTZ9, SAGE, CAGE, CT10, MART-1, immunoglobulin lambda-like polypeptide 1 (IGLL1), Hepatitis B Surface Antigen Binding Protein (HBsAg), viral capsid antigen (VCA), early antigen (EA), EBV nuclear antigen (EBNA), HHV-6 p41 early antigen, HHV-6B U94 latent antigen, HHV-6B p98 late antigen, cytomegalovirus (CMV) antigen, large T antigen, small T antigen, adenovirus antigen, respiratory syncytial virus (RSV) antigen, haemagglutinin (HA), neuraminidase (NA), parainfluenza type 1 antigen, parainfluenza type 2 antigen, parainfluenza type 3 antigen, parainfluenza type 4 antigen, Human Metapneumovirus (HMPV) antigen, hepatitis C virus (HCV) core antigen, HIV p24 antigen, human T-cell lympotrophic virus (HTLV-1) antigen, Merkel cell polyoma virus small T antigen, Merkel cell polyoma virus large T antigen, Kaposi sarcoma-associated herpesvirus (KSHV) lytic nuclear antigen and KSHV latent nuclear antigen.

As a non-limiting example, in some embodiments, the circular RNA and/or linear mRNA construct comprises an IRES and at least one expression sequence encoding a CAR targeting a cancer antigen. As a non-limiting example, in some embodiments, the circular RNA and/or linear mRNA construct comprises an IRES and a CAR comprising an antigen binding domain specific for CD19. In some embodiments, the circular RNA and/or linear mRNA construct comprises an IRES and a CAR comprising an antigen binding domain specific for BCMA. In some embodiments, the circular RNA and/or linear mRNA construct comprises an IRES and a CAR comprising an antigen binding domain specific for HER2. In some embodiments, the expression sequence is codon optimized.

As a non-limiting example, in some embodiments, the circular RNA and/or linear mRNA construct comprises a CAR comprising an antigen binding domain specific for CD19 (B-lymphocyte antigen CD19). CD19 is a biomarker for normal and neoplastic B cells, as well as follicular dendritic cells. Diffuse large B cell lymphoma (DLBCL) is the most common lymphoma, accounting for about 25% to 30% of all the non-Hodgkin lymphomas, followed by FL. As CD19 is expressed in over 95% of B-cell malignancies, it is an attractive target for immunotherapeutic approaches. One known example of a CAR T cell therapy targeting CD19 is Yescarta® (Kite Pharma Inc., axicabtagene ciloleucel), an anti-CD19 28-ζ (28-zeta) CAR. Another known example of a CAR T cell therapy targeting CD19 is Kymriah® (Novartis Pharmaceutical Corp., tisagenlecleucel), an anti-CD19 BB-ζ (BB-zeta) CAR. Accordingly, in some embodiments, the expression sequence of the circular RNA and/or linear mRNA construct encodes a CAR, where the codon is directed to an anti-CD19 domain known in the art. In some embodiments, the CAR construct comprises an anti-CD19 binder. In some embodiments, the expression sequence is codon optimized.

As a further non-limiting example, in some embodiments, the circular RNA and/or linear mRNA construct comprises a CAR comprising an antigen binding domain specific for Human Epidermal Growth Factor Receptor 2 (HER2). For example, the CAR can be directed to HER2-BBζ (BB-zeta) and/or HER2-28ζ (28-zeta). Accordingly, in some embodiments, the CAR construct comprises an anti-HER2 binder. In some embodiments, the expression sequence is codon optimized.

In some embodiments, the polynucleotide comprises an internal ribosome entry site (IRES) and expression sequence encoding an antigen binding molecule. In certain embodiments, the polynucleotide comprises the expression sequence and an IRES, wherein the IRES can facilitate expression of the antigen binding molecule or a subunit of the antigen binding molecule (e.g., an antigen binding domain) when delivered in vivo.

Lengthy table referenced here
US20260325678A1-20261022-T00001
Please refer to the end of the specification for access instructions.

In some embodiments, the circular RNA constructs, linear mRNA constructs, and related pharmaceutical compositions comprise the expression sequences described in Table 5, Table 6, Table 7, Table 9, Table 10 or a functional fragment thereof, e.g., a binding fragment, e.g., a set of complementarity determining regions (CDRs). In some embodiments, the circular RNA constructs, linear mRNA constructs, and related pharmaceutical compositions disclosed herein comprise an expression sequence that is at least 80%, 85%, 90%, 91%, 92%, 93%, 94%, 95%, 96%, 97%, 98%, or 99% identical to a sequence in Table 5, Table 6, Table 7, Table 9, Table 10, or a functional fragment thereof, e.g., a binding fragment, e.g., a set of complementarity determining regions (CDRs), wherein the expression sequence produces a protein having the desired sequence. Exemplary anti-CD19 binding or binder sequences are disclosed herein and known in the art, e.g., WO2012079000, WO2014153270, WO2017025038, WO2020123691, WO2014068079, WO2015158671, WO2016014565, WO2012079000, WO2014153270, WO2017025038, WO2020123691, WO2016090320, WO2017173349, WO2019006072, WO2019164891, WO2023098846, WO2018028647, WO2020018820, WO2020150339, WO2020243546, WO2020221873, WO2020038147, the contents of which are hereby incorporated by reference in their entireties.

The exemplary anti-CD19 binder sequences in Table 6 are codon-optimized and correspond to an anti-CD19 28ζ (28 zeta) CAR. The amino acid sequence corresponding to the nucleotide sequences in Table 6 is set forth below. In some embodiments, the circular RNA constructs, linear mRNA constructs, and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to said sequence or binding fragment thereof.

(SEQ ID NO: 280)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 4, an IRES from a construct of Table 5, and a CAR sequence encoding a polypeptide comprising at least 85%, 86%, 87%, 88%, 89%, 90%, 91%, 92%, 93%, 94%, 95%, 96%, 97%, 98%, 99%, or 100% sequence identity to the sequence above or binding fragment thereof. In some embodiments, said circular RNA and related pharmaceutical compositions further comprise a CD28z costimulatory domain as described herein and optionally exhibits increased activity compared to a suitable control having an alternate costimulatory domain. In some embodiments, said circular RNA further comprises a 4-1BB costimnulatory domain as described herein and optionally exhibits increased activity compared to a suitable control having an alternate costimulatory domain.

TABLE 6
Codon Optimized Sequences (anti-CD19 28-ζ)

| Codon No: | NUCLEOTIDE SEQUENCE |
|---|---|
| 2A-19 | ATGGCACTGCCCGTCACCGCACTCCTGCTCCCACTGGCACTGCTGCT |
| | CCATGCAGCTCGCCCCGATATCCAGATGACCCAGACCACCTCTAGCC |
| | TCAGCGCCTCTCTGGGTGACCGCGTCACCATCTCTTGCCGGGCCAGC |
| | CAAGACATCTCTAAGTACCTGAACTGGTACCAGCAGAAACCTGACG |
| | GAACCGTGAAGCTGCTGATCTACCACACCAGTCGGCTGCATTCCGGG |
| | GTGCCTTCCAGGTTCAGCGGTTCCGGCTCTGGGACCGATTATAGTCT |
| | CACCATCTCCAACCTCGAGCAGGAGGACATCGCAACCTACTTCTGCC |
| | AGCAGGGGAACACCCTGCCCTACACCTTCGGTGGCGGGACCAAGCT |
| | GGAGATCACTGGAGGTGGTGGCAGCGGAGGTGGAGGATCAGGTGGA |
| | GGCGGTAGCGAGGTGAAGCTGCAGGAGTCCGGACCTGGTCTGGTGG |
| | CCCCAAGCCAGTCCCTCAGCGTCACCTGCACAGTGTCCGGGGTGTCC |
| | CTGCCTGACTACGGTGTCTCCTGGATCAGGCAACCACCCCGGAAGGG |
| | TCTCGAGTGGCTGGGCGTCATCTGGGGCTCCGAGACCACCTACTACA |
| | ACAGCGCTCTGAAGTCCCGGCTGACCATCATCAAAGACAACTCCAA |
| | GAGCCAGGTGTTCTTGAAGATGAACTCCCTGCAAACCGATGACACC |
| | GCCATCTACTACTGCGCCAAGCACTACTACTATGGCGGTAGCTACGC |
| | CATGGATTATTGGGGTCAGGGCACCAGTGTCACCGTCTCCTCCATCG |
| | AGGTGATGTACCCTCCACCCTATCTGGACAACGAGAAGTCCAACGG |
| | CACCATCATCCACGTGAAGGGCAAGCACCTGTGCCCTAGCCCTCTGT |
| | TCCCAGGACCCTCCAAGCCCTTCTGGGTGCTGGTCGTGGTGGGAGGA |
| | GTCCTGGCCTGCTATTCCCTCCTCGTCACCGTGGCATTTATCATCTTC |
| | TGGGTCCGGAGCAAGCGGTCACGCCTGCTCCACTCCGACTACATGAA |
| | CATGACTCCTCGCAGACCTGGACCCACCCGGAAGCACTACCAGCCTT |
| | ATGCCCCACCCCGCGACTTTGCCGCTTACCGCTCTCGGGTCAAGTTC |
| | TCTCGGTCAGCAGACGCCCCTGCATACCAGCAGGGCCAGAACCAGC |
| | TGTATAACGAGCTGAACCTCGGCAGACGGGAGGAGTACGATGTGCT |
| | GGACAAGAGGAGAGGCAGAGACCCCGAGATGGGTGGTAAGCCACG |
| | GCGCAAGAACCCACAGGAGGGCTTGTACAACGAACTGCAGAAGGAC |
| | AAGATGGCCGAGGCCTACAGCGAGATCGGCATGAAGGGAGAGAGG |
| | CGCAGGGGCAAGGGTCACGACGGCCTGTACCAAGGGCTGTCCACCG |
| | CAACCAAGGACACCTACGATGCCCTGCACATGCAGGCCCTCCCACC |
| | AAGG (SEQ ID NO: 251) |
| 2A-20 | ATGGCACTCCCAGTCACCGCACTTCTGCTGCCTCTCGCCCTGCTGCTC |
| | CATGCAGCCAGACCCGACATCCAGATGACCCAAACCACCAGCTCCC |
| | TGTCCGCTTCCCTGGGTGACCGGGTGACTATCTCTTGCCGGGCCTCC |
| | CAAGACATCTCCAAGTACCTGAACTGGTATCAGCAAAAGCCTGACG |
| | GCACCGTCAAGCTCCTCATCTACCATACCTCCAGACTGCACTCCGGG |
| | GTGCCTAGCAGGTTCAGCGGAAGTGGGAGCGGCACCGACTACAGCC |
| | TCACCATCTCCAACCTGGAGCAGGAGGACATCGCCACCTACTTCTGC |
| | CAGCAGGGGAACACACTGCCCTACACCTTCGGCGGTGGCACCAAGC |
| | TGGAGATCACAGGTGGCGGAGGTTCCGGAGGAGGAGGTAGTGGAGG |
| | TGGAGGCAGCGAGGTGAAGCTCCAGGAATCCGGACCAGGTCTGGTG |
| | GCTCCCAGCCAGTCCCTCAGCGTGACCTGCACCGTGAGCGGCGTGTC |
| | TCTTCCCGATTACGGAGTGTCCTGGATCAGACAGCCACCCCGGAAGG |
| | GTCTGGAGTGGCTGGGAGTGATCTGGGGTTCCGAGACCACATACTAC |
| | AACTCAGCCCTCAAGAGCCGGCTCACCATCATCAAGGATAACTCCA |
| | AGTCCCAGGTCTTCCTGAAGATGAACTCTCTCCAGACCGACGACACC |
| | GCCATCTACTACTGCGCCAAGCACTACTACTACGGCGGGTCCTACGC |
| | CATGGACTACTGGGGTCAGGGAACCTCCGTCACCGTCAGCTCTATCG |
| | AGGTGATGTACCCTCCTCCCTACCTCGACAACGAGAAGAGCAACGG |
| | CACCATCATCCATGTGAAGGGGAAGCATCTCTGCCCCTCACCCCTGT |
| | TCCCCGGACCATCCAAGCCATTCTGGGTGCTGGTGGTTGTTGGTGGG |
| | GTCCTGGCTTGCTACTCACTCCTGGTCACCGTCGCCTTCATCATCTTC |
| | TGGGTGCGGTCAAAGAGGTCCCGGCTCTTGCACTCCGATTACATGAA |
| | CATGACTCCAAGGAGGCCTGGTCCCACACGGAAGCACTACCAACCA |
| | TATGCCCCACCACGCGACTTCGCTGCTTACCGGAGCCGGGTCAAGTT |
| | CAGTCGGAGTGCAGACGCCCCAGCCTACCAGCAGGGCCAGAACCAA |
| | CTCTACAACGAGCTTAATCTGGGTCGCCGGGAGGAGTATGACGTGCT |
| | CGATAAGAGAAGGGGCCGGGATCCTGAGATGGGCGGTAAGCCCAGA |
| | CGGAAGAACCCTCAGGAGGGGTTGTATAATGAGCTCCAGAAGGACA |
| | AGATGGCCGAGGCATACTCCGAGATCGGCATGAAAGGTGAGCGGAG |
| | GAGAGGCAAGGGGCATGACGGCCTGTACCAGGGGCTCAGCACAGCC |
| | ACCAAGGATACCTATGACGCACTCCACATGCAGGCACTGCCTCCACG |
| | G (SEQ ID NO: 301) |
| 2A-21 | ATGGCTCTGCCTGTGACAGCTCTGCTGCTGCCTCTGGCTCTGCTTCTG |
| | CATGCCGCCAGACCTGACATCCAGATGACCCAGACAACCAGCAGCC |
| | TGTCTGCCAGCCTGGGCGATAGAGTGACCATCAGCTGTAGAGCCAG |
| | CCAGGACATCAGCAAGTACCTGAACTGGTATCAGCAGAAACCCGAC |
| | GGCACCGTGAAGCTGCTGATCTACCACACCAGCAGACTGCACAGCG |
| | GCGTGCCAAGCAGATTTTCTGGCAGCGGCTCTGGCACCGACTACAGC |
| | CTGACAATCAGCAACCTGGAACAAGAGGATATCGCTACCTACTTCTG |
| | CCAGCAAGGCAACACCCTGCCTTACACCTTTGGCGGAGGCACCAAG |
| | CTGGAAATCACAGGCGGCGGAGGAAGCGGAGGCGGAGGATCTGGT |
| | GGTGGTGGATCTGAAGTGAAACTGCAAGAGTCTGGCCCTGGCCTGG |
| | TGGCCCCATCTCAATCTCTGAGCGTGACCTGTACCGTCAGCGGAGTG |
| | TCCCTGCCTGATTATGGCGTGTCCTGGATCCGGCAGCCTCCTAGAAA |
| | AGGCCTGGAATGGCTGGGCGTGATCTGGGGCAGCGAGACAACCTAC |
| | TACAACAGCGCCCTGAAGTCCCGGCTGACCATCATCAAGGACAACT |
| | CCAAGAGCCAGGTGTTCCTGAAGATGAACAGCCTGCAGACCGACGA |
| | CACCGCCATCTACTATTGCGCCAAGCACTACTACTACGGCGGCAGCT |
| | ACGCCATGGATTATTGGGGCCAGGGCACCAGCGTGACCGTGTCTAG |
| | CATCGAAGTGATGTACCCTCCACCTTACCTGGACAACGAGAAGTCCA |
| | ACGGCACCATCATCCACGTGAAGGGCAAGCACCTGTGTCCTTCTCCA |
| | CTGTTCCCCGGACCTAGCAAGCCTTTCTGGGTGCTCGTTGTTGTTGGC |
| | GGCGTGCTGGCCTGTTACTCTCTGCTGGTTACCGTGGCCTTCATCATC |
| | TTTTGGGTCCGAAGCAAGCGGAGCCGGCTGCTGCACTCCGACTACAT |
| | GAACATGACCCCTAGACGGCCCGGACCAACCAGAAAGCACTACCAG |
| | CCTTACGCTCCTCCTAGAGACTTCGCCGCCTACCGGTCCAGAGTGAA |
| | GTTCAGCAGATCCGCCGATGCTCCCGCCTATCAGCAGGGCCAAAACC |
| | AGCTGTACAACGAGCTGAACCTGGGGAGAAGAGAAGAGTACGACGT |
| | GCTGGACAAGCGGAGAGGCAGAGATCCTGAAATGGGCGGCAAGCCC |
| | AGACGGAAGAATCCTCAAGAGGGCCTGTATAATGAGCTGCAGAAAG |
| | ACAAGATGGCCGAGGCCTACAGCGAGATCGGAATGAAGGGCGAGC |
| | GCAGAAGAGGCAAGGGACACGATGGACTGTACCAGGGCCTGAGCAC |
| | CGCCACCAAGGATACCTATGATGCCCTGCACATGCAGGCCCTGCCTC |
| | CAAGA (SEQ ID NO: 244) |
| 2A-22 | ATGGCCCTTCCCGTCACCGCTCTCCTCCTGCCACTGGCCTTGCTGCTG |
| | CACGCTGCACGGCCAGACATCCAGATGACCCAGACAACCAGCTCTC |
| | TGTCAGCCTCTCTCGGCGATCGCGTCACAATCAGCTGCCGCGCTTCC |
| | CAAGACATCTCCAAGTACCTGAACTGGTACCAGCAAAAGCCCGACG |
| | GCACCGTGAAGCTGCTCATCTACCACACCTCCAGACTGCATAGCGGG |
| | GTGCCCAGCAGATTCAGTGGCTCAGGCTCAGGCACCGACTACAGCCT |
| | GACCATCTCCAACCTGGAGCAGGAGGACATTGCCACATACTTCTGCC |
| | AGCAGGGCAACACCCTGCCCTACACCTTCGGAGGCGGCACAAAGCT |
| | GGAGATCACCGGTGGAGGAGGGAGTGGAGGAGGAGGCAGTGGTGG |
| | CGGAGGTTCCGAGGTGAAGCTCCAGGAATCAGGTCCAGGACTGGTC |
| | GCCCCTTCCCAGTCCCTGTCCGTCACCTGCACCGTGAGTGGCGTCAG |
| | CCTCCCAGACTACGGTGTGTCTTGGATCCGCCAACCTCCTCGCAAAG |
| | GCCTGGAATGGCTCGGCGTCATCTGGGGAAGCGAGACAACCTACTA |
| | TAACTCCGCACTGAAGTCCCGCCTCACCATCATCAAGGATAATAGCA |
| | AGAGCCAGGTCTTCCTCAAGATGAACTCCCTGCAGACCGACGATACC |
| | GCCATCTACTACTGTGCCAAGCACTACTACTACGGAGGTTCTTACGC |
| | CATGGATTACTGGGGACAGGGAACCTCTGTCACCGTCAGCTCCATCG |
| | AGGTCATGTATCCACCACCCTACCTGGACAACGAAAAGAGCAATGG |
| | CACCATCATCCACGTGAAGGGGAAGCACCTCTGCCCCTCACCCCTGT |
| | TCCCTGGTCCCTCCAAGCCTTTCTGGGTCCTGGTCGTCGTGGGAGGC |
| | GTGTTGGCCTGTTACTCCCTGCTCGTCACCGTCGCCTTCATCATCTTC |
| | TGGGTTAGGAGTAAGCGGTCCCGGCTTCTGCACTCTGACTACATGAA |
| | CATGACACCCAGAAGACCTGGGCCAACCCGGAAGCACTACCAGCCC |
| | TACGCTCCACCCAGGGACTTTGCAGCCTACAGGTCCCGCGTCAAGTT |
| | CTCCCGGTCTGCTGACGCACCTGCCTACCAGCAGGGCCAAAACCAGC |
| | TCTACAACGAGTTGAACCTCGGCAGACGGGAGGAGTACGACGTCCT |
| | CGACAAAAGGCGGGGTCGGGATCCTGAGATGGGCGGTAAGCCAAGG |
| | CGGAAGAACCCACAGGAAGGCCTCTATAATGAGCTCCAGAAGGATA |
| | AGATGGCTGAGGCCTACTCCGAGATCGGGATGAAGGGCGAAAGGAG |
| | ACGGGGTAAGGGGCACGACGGCCTCTATCAGGGTCTGAGCACCGCC |
| | ACCAAGGACACCTACGACGCCCTGCACATGCAGGCACTGCCACCTC |
| | GG (SEQ ID NO: 302) |
| 2A-23 | ATGGCTCTGCCAGTGACCGCACTGCTGCTGCCCTTAGCCTTACTCCTT |
| | CACGCAGCCAGGCCCGACATCCAGATGACCCAGACCACCAGCTCCC |
| | TTTCCGCAAGCCTCGGCGACAGGGTCACCATCTCCTGTCGGGCCAGC |
| | CAGGACATCAGCAAGTACCTGAACTGGTACCAGCAGAAGCCCGACG |
| | GCACCGTGAAGCTGCTGATCTACCACACCTCACGGCTGCACTCAGGC |
| | GTGCCCTCACGGTTTAGCGGATCAGGCAGCGGCACCGACTACAGCCT |
| | GACTATCAGCAACCTGGAGCAGGAGGACATCGCCACCTACTTCTGCC |
| | AGCAGGGCAACACCCTGCCCTACACCTTCGGAGGCGGCACCAAGCT |
| | GGAGATCACCGGTGGCGGTGGTTCAGGTGGCGGAGGCTCAGGAGGA |
| | GGCGGCAGCGAGGTGAAGCTGCAGGAGTCAGGTCCAGGACTGGTGG |
| | CACCCAGCCAGAGCCTGAGCGTGACTTGCACCGTGTCAGGCGTGAG |
| | CCTGCCAGACTACGGCGTGAGCTGGATCCGGCAGCCTCCTCGGAAG |
| | GGCTTAGAGTGGCTGGGCGTGATCTGGGGCAGCGAGACCACCTACT |
| | ACAACTCAGCCCTGAAGAGCCGGCTGACCATCATCAAGGACAACAG |
| | CAAGAGCCAGGTGTTCCTGAAGATGAACAGCCTGCAGACCGACGAC |
| | ACCGCCATCTACTACTGCGCCAAGCACTACTACTACGGCGGCAGCTA |
| | CGCCATGGACTACTGGGGACAGGGTACCAGCGTGACCGTGAGCAGC |
| | ATCGAGGTGATGTACCCTCCTCCCTACCTGGACAACGAGAAGAGCA |
| | ACGGCACCATCATCCACGTGAAGGGCAAGCACCTGTGCCCTAGCCCT |
| | TTATTCCCCGGCCCCTCAAAACCCTTCTGGGTGCTGGTCGTCGTCGGT |
| | GGCGTGCTGGCATGCTACAGCCTGCTGGTGACCGTGGCCTTCATCAT |
| | ATTCTGGGTCCGGTCAAAGCGGAGCCGGTTACTGCACAGCGACTAC |
| | ATGAACATGACTCCACGGCGTCCAGGTCCCACTCGGAAGCACTACC |
| | AACCCTACGCTCCTCCCCGTGACTTTGCTGCCTACCGTAGCCGGGTG |
| | AAGTTCTCCAGGAGCGCCGATGCCCCAGCCTACCAGCAGGGCCAGA |
| | ACCAGCTCTACAATGAGCTTAACCTTGGCAGGCGGGAGGAGTACGA |
| | CGTGCTGGACAAGAGGAGGGGCCGTGATCCCGAGATGGGAGGCAAG |
| | CCCCGTAGGAAGAATCCCCAGGAGGGCCTTTACAACGAGCTCCAGA |
| | AGGACAAGATGGCCGAGGCCTACAGCGAGATCGGCATGAAGGGAG |
| | AGCGTAGGCGTGGAAAGGGCCACGACGGCCTGTACCAGGGCCTGAG |
| | CACTGCTACCAAGGACACCTACGACGCCCTGCACATGCAGGCTCTTC |
| | CACCCCGG (SEQ ID NO: 303) |

Table 7 sets forth nucleotide and amino acid sequences for additional exemplary anti-CD19 binder sequences that are not codon-optimized. The sequences are directed to an anti-CD19 28ζ (28 zeta) CAR. In some embodiments, the circular RNA constructs, linear mRNA, and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to any one of the CAR sequences of Table 7 or binding fragments thereof.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 4, an IRES from a construct of Table 5, and a CAR sequence encoding a polypeptide comprising at least 85%, 86%, 87%, 88%, 89%, 90%, 91%, 92%, 93%, 94%, 95%, 96%, 97%, 98%, 99%, or 100% sequence identity to any one of the CAR sequences of Table 6 or Table 7 or binding fragments thereof. In some embodiments, said circular RNA and related pharmaceutical compositions further comprise a CD28z costimulatory domain as described herein and optionally exhibits increased activity compared to a suitable control having an alternate costimulatory domain. In some embodiments, said circular RNA further comprises a 4-1BB costimulatory domain as described herein and optionally exhibits increased activity compared to a suitable control having an alternate costimulatory domain.

TABLE 7
Additional Codon Amino Acid and Nucleotide Sequences (anti-CD19 28-ζ)

| | Codon amino acid |
|---|---|
| Codon nucleotide sequence | sequence |
| ATGCTCCTCCTGGTGACCAGCTTGCTCCTGTGCGAACTGC | MLLLVTSLLLCELP |
| CACACCCCGCCTTCCTCCTCATCCCCGATATCCAGATGAC | HPAFLLIPDIQMTQ |
| CCAGACCACCTCCTCCCTGAGCGCAAGCCTCGGCGATCG | TTSSLSASLGDRVTI |
| GGTGACCATCTCATGCAGGGCCTCCCAGGACATCTCCAA | SCRASQDISKYLNW |
| GTATCTGAACTGGTATCAGCAGAAGCCTGACGGCACCGT | YQQKPDGTVKLLIY |
| CAAGCTGCTCATCTACCACACCTCACGGCTGCACTCAGGC | HTSRLHSGVPSRFS |
| GTCCCCTCAAGATTCAGCGGTAGCGGATCCGGGACCGAC | GSGSGTDYSLTISN |
| TACTCCCTTACCATCAGCAACCTGGAGCAGGAGGATATC | LEQEDIATYFCQQG |
| GCCACATACTTCTGCCAGCAGGGTAACACCCTGCCCTATA | NTLPYTFGGGTKLE |
| CCTTCGGCGGTGGGACCAAGCTGGAGATCACCGGTTCTA | ITGSTSGSGKPGSG |
| CATCCGGATCCGGCAAGCCTGGTAGTGGCGAGGGCTCCA | EGSTKGEVKLQESG |
| CCAAAGGGGAGGTGAAGCTGCAGGAGTCCGGTCCAGGTC | PGLVAPSQSLSVTC |
| TGGTGGCTCCAAGTCAGTCCCTGTCTGTGACTTGCACCGT | TVSGVSLPDYGVS |
| GTCAGGCGTGAGCCTGCCTGACTACGGGGTGAGCTGGAT | WIRQPPRKGLEWL |
| CCGGCAGCCACCTCGGAAGGGGTTGGAGTGGCTGGGAGT | GVIWGSETTYYNS |
| CATCTGGGGATCCGAGACCACCTACTACAATTCCGCCCTC | ALKSRLTIIKDNSKS |
| AAAAGCCGCCTCACCATCATCAAGGACAACTCCAAGTCC | QVFLKMNSLQTDD |
| CAGGTCTTCCTGAAGATGAATTCCCTGCAGACCGACGAC | TAIYYCAKHYYYG |
| ACCGCTATCTATTACTGCGCCAAGCATTACTACTACGGCG | GSYAMDYWGQGT |
| GGTCCTACGCCATGGACTACTGGGGTCAAGGCACCTCCG | SVTVSSAAAIEVMY |
| TCACTGTTTCCTCCGCAGCAGCCATCGAGGTCATGTATCC | PPPYLDNEKSNGTII |
| TCCTCCCTACCTCGACAACGAGAAGTCCAACGGGACCAT | HVKGKHLCPSPLFP |
| CATCCACGTGAAGGGCAAGCACCTCTGCCCAAGCCCACT | GPSKPFWVLVVVG |
| GTTCCCAGGGCCCTCCAAACCATTCTGGGTGCTCGTGGTG | GVLACYSLLVTVA |
| GTGGGTGGCGTGCTCGCTTGCTACTCCCTCCTGGTCACCG | FIIFWVRSKRSRLLH |
| TCGCCTTCATCATCTTTTGGGTCCGGAGTAAGCGCAGCCG | SDYMNMTPRRPGP |
| CCTGCTCCATAGCGACTACATGAACATGACCCCACGGAG | TRKHYQPYAPPRDF |
| ACCTGGTCCCACCCGGAAACACTACCAGCCCTACGCACC | AAYRSRVKESRSA |
| ACCCAGGGACTTCGCTGCCTATCGGTCCCGGGTTAAATTC | DAPAYQQGONQLY |
| TCTAGGTCCGCTGATGCCCCAGCCTACCAGCAGGGCCAG | NELNLGRREEYDV |
| AACCAGCTGTACAATGAGCTGAACCTGGGTAGACGGGAG | LDKRRGRDPEMGG |
| GAGTATGACGTCCTGGATAAGCGCAGAGGGAGAGACCCC | KPRRKNPQEGLYN |
| GAGATGGGTGGAAAGCCCAGGCGGAAGAATCCCCAGGA | ELQKDKMAEAYSE |
| GGGTCTCTATAACGAGCTCCAGAAGGACAAGATGGCCGA | IGMKGERRRGKGH |
| GGCCTACAGCGAGATCGGGATGAAAGGGGAAAGAAGGC | DGLYQGLSTATKD |
| GGGGAAAGGGCCATGACGGACTGTACCAGGGTCTGTCCA | TYDALHMQALPPR |
| CCGCTACCAAGGACACCTACGATGCACTGCACATGCAGG | (SEQ ID NO: 309) |
| CACTGCCTCCTCGG (SEQ ID NO: 304) | |
| ATGCTGCTTCTCGTTACATCTCTGTTGCTCTGCGAGCTGCC | MLLLVTSLLLCELP |
| TCATCCAGCCTTCCTCCTGATTCCCGATATCCAGATGACC | HPAFLLIPDIQMTQ |
| CAGACCACCTCTAGCCTCAGCGCCTCTCTGGGTGACCGCG | TTSSLSASLGDRVTI |
| TCACCATCTCTTGCCGGGCCAGCCAAGACATCTCTAAGTA | SCRASQDISKYLNW |
| CCTGAACTGGTACCAGCAGAAACCTGACGGAACCGTGAA | YQQKPDGTVKLLIY |
| GCTGCTGATCTACCACACCAGTCGGCTGCATTCCGGGGTG | HTSRLHSGVPSRFS |
| CCTTCCAGGTTCAGCGGTTCCGGCTCTGGGACCGATTATA | GSGSGTDYSLTISN |
| GTCTCACCATCTCCAACCTCGAGCAGGAGGACATCGCAA | LEQEDIATYFCQQG |
| CCTACTTCTGCCAGCAGGGGAACACCCTGCCCTACACCTT | NTLPYTFGGGTKLE |
| CGGTGGCGGGACCAAGCTGGAGATCACTGGCAGCACCTC | ITGSTSGSGKPGSG |
| AGGCTCTGGGAAGCCTGGCAGCGGTGAAGGCAGCACCAA | EGSTKGEVKLQESG |
| GGGTGAGGTGAAGCTGCAGGAGTCCGGACCTGGTCTGGT | PGLVAPSQSLSVTC |
| GGCCCCAAGCCAGTCCCTCAGCGTCACCTGCACAGTGTC | TVSGVSLPDYGVS |
| CGGGGTGTCCCTGCCTGACTACGGTGTCTCCTGGATCAGG | WIRQPPRKGLEWL |
| CAACCACCCCGGAAGGGTCTCGAGTGGCTGGGCGTCATC | GVIWGSETTYYNS |
| TGGGGCTCCGAGACCACCTACTACAACAGCGCTCTGAAG | ALKSRLTIIKDNSKS |
| TCCCGGCTGACCATCATCAAAGACAACTCCAAGAGCCAG | QVFLKMNSLQTDD |
| GTGTTCTTGAAGATGAACTCCCTGCAAACCGATGACACC | TAIYYCAKHYYYG |
| GCCATCTACTACTGCGCCAAGCACTACTACTATGGCGGTA | GSYAMDYWGQGT |
| GCTACGCCATGGATTATTGGGGTCAGGGCACCAGTGTCA | SVTVSSAAAIEVMY |
| CCGTCTCCTCCGCTGCCGCTATCGAGGTGATGTACCCTCC | PPPYLDNEKSNGTII |
| ACCCTATCTGGACAACGAGAAGTCCAACGGCACCATCAT | HVKGKHLCPSPLFP |
| CCACGTGAAGGGCAAGCACCTGTGCCCTAGCCCTCTGTTC | GPSKPFWVLVVVG |
| CCAGGACCCTCCAAGCCCTTCTGGGTGCTGGTCGTGGTGG | GVLACYSLLVTVA |
| GAGGAGTCCTGGCCTGCTATTCCCTCCTCGTCACCGTGGC | FIIFWVRSKRSRLLH |
| ATTTATCATCTTCTGGGTCCGGAGCAAGCGGTCACGCCTG | SDYMNMTPRRPGP |
| CTCCACTCCGACTACATGAACATGACTCCTCGCAGACCTG | TRKHYQPYAPPRDF |
| GACCCACCCGGAAGCACTACCAGCCTTATGCCCCACCCC | AAYRSRVKFSRSA |
| GCGACTTTGCCGCTTACCGCTCTCGGGTCAAGTTCTCTCG | DAPAYQQGQNQLY |
| GTCAGCAGACGCCCCTGCATACCAGCAGGGCCAGAACCA | NELNLGRREEYDV |
| GCTGTATAACGAGCTGAACCTCGGCAGACGGGAGGAGTA | LDKRRGRDPEMGG |
| CGATGTGCTGGACAAGAGGAGAGGCAGAGACCCCGAGA | KPRRKNPQEGLYN |
| TGGGTGGTAAGCCACGGCGCAAGAACCCACAGGAGGGCT | ELQKDKMAEAYSE |
| TGTACAACGAACTGCAGAAGGACAAGATGGCCGAGGCCT | IGMKGERRRGKGH |
| ACAGCGAGATCGGCATGAAGGGAGAGAGGCGCAGGGGC | DGLYQGLSTATKD |
| AAGGGTCACGACGGCCTGTACCAAGGGCTGTCCACCGCA | TYDALHMQALPPR |
| ACCAAGGACACCTACGATGCCCTGCACATGCAGGCCCTC | (SEQ ID NO: 309) |
| CCACCAAGG (SEQ ID NO: 305) | |
| ATGGCACTTCCAGTTACAGCACTTCTGCTTCCATTGGCAC | MALPVTALLLPLAL |
| TGCTGCTCCATGCAGCTCGCCCCGATATCCAGATGACCCA | LLHAARPDIQMTQT |
| GACCACCTCTAGCCTCAGCGCCTCTCTGGGTGACCGCGTC | TSSLSASLGDRVTIS |
| ACCATCTCTTGCCGGGCCAGCCAAGACATCTCTAAGTACC | CRASQDISKYLNW |
| TGAACTGGTACCAGCAGAAACCTGACGGAACCGTGAAGC | YQQKPDGTVKLLIY |
| TGCTGATCTACCACACCAGTCGGCTGCATTCCGGGGTGCC | HTSRLHSGVPSRFS |
| TTCCAGGTTCAGCGGTTCCGGCTCTGGGACCGATTATAGT | GSGSGTDYSLTISN |
| CTCACCATCTCCAACCTCGAGCAGGAGGACATCGCAACC | LEQEDIATYFCQQG |
| TACTTCTGCCAGCAGGGGAACACCCTGCCCTACACCTTCG | NTLPYTFGGGTKLE |
| GTGGCGGGACCAAGCTGGAGATCACTGGCAGCACCTCAG | ITGSTSGSGKPGSG |
| GCTCTGGGAAGCCTGGCAGCGGTGAAGGCAGCACCAAGG | EGSTKGEVKLQESG |
| GTGAGGTGAAGCTGCAGGAGTCCGGACCTGGTCTGGTGG | PGLVAPSQSLSVTC |
| CCCCAAGCCAGTCCCTCAGCGTCACCTGCACAGTGTCCG | TVSGVSLPDYGVS |
| GGGTGTCCCTGCCTGACTACGGTGTCTCCTGGATCAGGCA | WIRQPPRKGLEWL |
| ACCACCCCGGAAGGGTCTCGAGTGGCTGGGCGTCATCTG | GVIWGSETTYYNS |
| GGGCTCCGAGACCACCTACTACAACAGCGCTCTGAAGTC | ALKSRLTIIKDNSKS |
| CCGGCTGACCATCATCAAAGACAACTCCAAGAGCCAGGT | QVFLKMNSLQTDD |
| GTTCTTGAAGATGAACTCCCTGCAAACCGATGACACCGC | TAIYYCAKHYYYG |
| CATCTACTACTGCGCCAAGCACTACTACTATGGCGGTAGC | GSYAMDYWGQGT |
| TACGCCATGGATTATTGGGGTCAGGGCACCAGTGTCACC | SVTVSSAAAIEVMY |
| GTCTCCTCCGCTGCCGCTATCGAGGTGATGTACCCTCCAC | PPPYLDNEKSNGTII |
| CCTATCTGGACAACGAGAAGTCCAACGGCACCATCATCC | HVKGKHLCPSPLFP |
| ACGTGAAGGGCAAGCACCTGTGCCCTAGCCCTCTGTTCCC | GPSKPFWVLVVVG |
| AGGACCCTCCAAGCCCTTCTGGGTGCTGGTCGTGGTGGG | GVLACYSLLVTVA |
| AGGAGTCCTGGCCTGCTATTCCCTCCTCGTCACCGTGGCA | FIIFWVRSKRSRLLH |
| TTTATCATCTTCTGGGTCCGGAGCAAGCGGTCACGCCTGC | SDYMNMTPRRPGP |
| TCCACTCCGACTACATGAACATGACTCCTCGCAGACCTGG | TRKHYQPYAPPRDF |
| ACCCACCCGGAAGCACTACCAGCCTTATGCCCCACCCCG | AAYRSRVKFSRSA |
| CGACTTTGCCGCTTACCGCTCTCGGGTCAAGTTCTCTCGG | DAPAYQQGQNQLY |
| TCAGCAGACGCCCCTGCATACCAGCAGGGCCAGAACCAG | NELNLGRREEYDV |
| CTGTATAACGAGCTGAACCTCGGCAGACGGGAGGAGTAC | LDKRRGRDPEMGG |
| GATGTGCTGGACAAGAGGAGAGGCAGAGACCCCGAGAT | KPRRKNPQEGLYN |
| GGGTGGTAAGCCACGGCGCAAGAACCCACAGGAGGGCTT | ELQKDKMAEAYSE |
| GTACAACGAACTGCAGAAGGACAAGATGGCCGAGGCCTA | IGMKGERRRGKGH |
| CAGCGAGATCGGCATGAAGGGAGAGAGGCGCAGGGGCA | DGLYQGLSTATKD |
| AGGGTCACGACGGCCTGTACCAAGGGCTGTCCACCGCAA | TYDALHMQALPPR |
| CCAAGGACACCTACGATGCCCTGCACATGCAGGCCCTCC | (SEQ ID NO: 310) |
| CACCAAGG (SEQ ID NO: 306) | |
| ATGGCACTGCCCGTCACCGCACTCCTGCTCCCACTGGCAC | MALPVTALLLPLAL |
| TGCTGCTCCATGCAGCTCGCCCCGATATCCAGATGACCCA | LLHAARPDIQMTQT |
| GACCACCTCTAGCCTCAGCGCCTCTCTGGGTGACCGCGTC | TSSLSASLGDRVTIS |
| ACCATCTCTTGCCGGGCCAGCCAAGACATCTCTAAGTACC | CRASQDISKYLNW |
| TGAACTGGTACCAGCAGAAACCTGACGGAACCGTGAAGC | YQQKPDGTVKLLIY |
| TGCTGATCTACCACACCAGTCGGCTGCATTCCGGGGTGCC | HTSRLHSGVPSRFS |
| TTCCAGGTTCAGCGGTTCCGGCTCTGGGACCGATTATAGT | GSGSGTDYSLTISN |
| CTCACCATCTCCAACCTCGAGCAGGAGGACATCGCAACC | LEQEDIATYFCQQG |
| TACTTCTGCCAGCAGGGGAACACCCTGCCCTACACCTTCG | NTLPYTFGGGTKLE |
| GTGGCGGGACCAAGCTGGAGATCACTGGCAGCACCTCAG | ITGSTSGSGKPGSG |
| GCTCTGGGAAGCCTGGCAGCGGTGAAGGCAGCACCAAGG | EGSTKGEVKLQESG |
| GTGAGGTGAAGCTGCAGGAGTCCGGACCTGGTCTGGTGG | PGLVAPSQSLSVTC |
| CCCCAAGCCAGTCCCTCAGCGTCACCTGCACAGTGTCCG | TVSGVSLPDYGVS |
| GGGTGTCCCTGCCTGACTACGGTGTCTCCTGGATCAGGCA | WIRQPPRKGLEWL |
| ACCACCCCGGAAGGGTCTCGAGTGGCTGGGCGTCATCTG | GVIWGSETTYYNS |
| GGGCTCCGAGACCACCTACTACAACAGCGCTCTGAAGTC | ALKSRLTIIKDNSKS |
| CCGGCTGACCATCATCAAAGACAACTCCAAGAGCCAGGT | QVFLKMNSLQTDD |
| GTTCTTGAAGATGAACTCCCTGCAAACCGATGACACCGC | TAIYYCAKHYYYG |
| CATCTACTACTGCGCCAAGCACTACTACTATGGCGGTAGC | GSYAMDYWGQGT |
| TACGCCATGGATTATTGGGGTCAGGGCACCAGTGTCACC | SVTVSSAAAIEVMY |
| GTCTCCTCCGCTGCCGCTATCGAGGTGATGTACCCTCCAC | PPPYLDNEKSNGTII |
| CCTATCTGGACAACGAGAAGTCCAACGGCACCATCATCC | HVKGKHLCPSPLFP |
| ACGTGAAGGGCAAGCACCTGTGCCCTAGCCCTCTGTTCCC | GPSKPFWVLVVVG |
| AGGACCCTCCAAGCCCTTCTGGGTGCTGGTCGTGGTGGG | GVLACYSLLVTVA |
| AGGAGTCCTGGCCTGCTATTCCCTCCTCGTCACCGTGGCA | FIIFWVRSKRSRLLH |
| TTTATCATCTTCTGGGTCCGGAGCAAGCGGTCACGCCTGC | SDYMNMTPRRPGP |
| TCCACTCCGACTACATGAACATGACTCCTCGCAGACCTGG | TRKHYQPYAPPRDF |
| ACCCACCCGGAAGCACTACCAGCCTTATGCCCCACCCCG | AAYRSRVKFSRSA |
| CGACTTTGCCGCTTACCGCTCTCGGGTCAAGTTCTCTCGG | DAPAYQQGQNQLY |
| TCAGCAGACGCCCCTGCATACCAGCAGGGCCAGAACCAG | NELNLGRREEYDV |
| CTGTATAACGAGCTGAACCTCGGCAGACGGGAGGAGTAC | LDKRRGRDPEMGG |
| GATGTGCTGGACAAGAGGAGAGGCAGAGACCCCGAGAT | KPRRKNPQEGLYN |
| GGGTGGTAAGCCACGGCGCAAGAACCCACAGGAGGGCTT | ELQKDKMAEAYSE |
| GTACAACGAACTGCAGAAGGACAAGATGGCCGAGGCCTA | IGMKGERRRGKGH |
| CAGCGAGATCGGCATGAAGGGAGAGAGGCGCAGGGGCA | DGLYQGLSTATKD |
| AGGGTCACGACGGCCTGTACCAAGGGCTGTCCACCGCAA | TYDALHMQALPPR |
| CCAAGGACACCTACGATGCCCTGCACATGCAGGCCCTCC | (SEQ ID NO: 310) |
| CACCAAGG (SEQ ID NO: 307) | |
| ATGGCACTTCCAGTTACAGCACTTCTGCTTCCATTGGCAC | MALPVTALLLPLAL |
| TGCTGCTCCATGCAGCTCGCCCCGATATCCAGATGACCCA | LLHAARPDIQMTQT |
| GACCACCTCTAGCCTCAGCGCCTCTCTGGGTGACCGCGTC | TSSLSASLGDRVTIS |
| ACCATCTCTTGCCGGGCCAGCCAAGACATCTCTAAGTACC | CRASQDISKYLNW |
| TGAACTGGTACCAGCAGAAACCTGACGGAACCGTGAAGC | YQQKPDGTVKLLIY |
| TGCTGATCTACCACACCAGTCGGCTGCATTCCGGGGTGCC | HTSRLHSGVPSRFS |
| TTCCAGGTTCAGCGGTTCCGGCTCTGGGACCGATTATAGT | GSGSGTDYSLTISN |
| CTCACCATCTCCAACCTCGAGCAGGAGGACATCGCAACC | LEQEDIATYFCQQG |
| TACTTCTGCCAGCAGGGGAACACCCTGCCCTACACCTTCG | NTLPYTFGGGTKLE |
| GTGGCGGGACCAAGCTGGAGATCACTGGAGGTGGTGGCA | ITGGGGSGGGGSG |
| GCGGAGGTGGAGGATCAGGTGGAGGCGGTAGCGAGGTG | GGGSEVKLQESGP |
| AAGCTGCAGGAGTCCGGACCTGGTCTGGTGGCCCCAAGC | GLVAPSQSLSVTCT |
| CAGTCCCTCAGCGTCACCTGCACAGTGTCCGGGGTGTCCC | VSGVSLPDYGVSWI |
| TGCCTGACTACGGTGTCTCCTGGATCAGGCAACCACCCCG | RQPPRKGLEWLGVI |
| GAAGGGTCTCGAGTGGCTGGGCGTCATCTGGGGCTCCGA | WGSETTYYNSALK |
| GACCACCTACTACAACAGCGCTCTGAAGTCCCGGCTGAC | SRLTIIKDNSKSQVF |
| CATCATCAAAGACAACTCCAAGAGCCAGGTGTTCTTGAA | LKMNSLQTDDTAI |
| GATGAACTCCCTGCAAACCGATGACACCGCCATCTACTA | YYCAKHYYYGGSY |
| CTGCGCCAAGCACTACTACTATGGCGGTAGCTACGCCAT | AMDYWGQGTSVT |
| GGATTATTGGGGTCAGGGCACCAGTGTCACCGTCTCCTCC | VSSIEVMYPPPYLD |
| ATCGAGGTGATGTACCCTCCACCCTATCTGGACAACGAG | NEKSNGTIIHVKGK |
| AAGTCCAACGGCACCATCATCCACGTGAAGGGCAAGCAC | HLCPSPLFPGPSKPF |
| CTGTGCCCTAGCCCTCTGTTCCCAGGACCCTCCAAGCCCT | WVLVVVGGVLAC |
| TCTGGGTGCTGGTCGTGGTGGGAGGAGTCCTGGCCTGCTA | YSLLVTVAFIIFWV |
| TTCCCTCCTCGTCACCGTGGCATTTATCATCTTCTGGGTCC | RSKRSRLLHSDYM |
| GGAGCAAGCGGTCACGCCTGCTCCACTCCGACTACATGA | NMTPRRPGPTRKH |
| ACATGACTCCTCGCAGACCTGGACCCACCCGGAAGCACT | YQPYAPPRDFAAY |
| ACCAGCCTTATGCCCCACCCCGCGACTTTGCCGCTTACCG | RSRVKFSRSADAPA |
| CTCTCGGGTCAAGTTCTCTCGGTCAGCAGACGCCCCTGCA | YQQGQNQLYNELN |
| TACCAGCAGGGCCAGAACCAGCTGTATAACGAGCTGAAC | LGRREEYDVLDKR |
| CTCGGCAGACGGGAGGAGTACGATGTGCTGGACAAGAGG | RGRDPEMGGKPRR |
| AGAGGCAGAGACCCCGAGATGGGTGGTAAGCCACGGCG | KNPQEGLYNELQK |
| CAAGAACCCACAGGAGGGCTTGTACAACGAACTGCAGAA | DKMAEAYSEIGMK |
| GGACAAGATGGCCGAGGCCTACAGCGAGATCGGCATGAA | GERRRGKGHDGLY |
| GGGAGAGAGGCGCAGGGGCAAGGGTCACGACGGCCTGT | QGLSTATKDTYDA |
| ACCAAGGGCTGTCCACCGCAACCAAGGACACCTACGATG | LHMQALPPR (SEQ |
| CCCTGCACATGCAGGCCCTCCCACCAAGG (SEQ ID NO: | ID NO: 280) |
| 308) | |

Table 9 sets forth nucleotide and amino acid sequences for additional exemplary binder sequences that are not codon-optimized, including a mouse anti-CD19 binder, anti-BCMA binders, and anti-HER2 binders. In some embodiments, the circular RNA constructs, linear mRNA constructs, and related pharmaceutical compositions disclosed herein comprise a CAR sequence encoding a polypeptide that comprises at least 80%, 85%, 90%, 91%, 92%, 93%, 94%, 95%, 96%, 97%, 98%, or 99% identity to any one of the CAR sequences of Table 9 or binding fragments thereof.

Table 10 sets forth amino acid sequences for exemplary anti-CD19 binders. In some embodiments, the circular RNA constructs, linear mRNA constructs, and related pharmaceutical compositions disclosed herein comprise an expression sequence encoding a CD19 binding molecule. In some embodiments, the circular RNA constructs, linear mRNA constructs, and related pharmaceutical compositions disclosed herein comprise an expression sequence encoding a CD19 binding molecule encoding a polypeptide that comprises at least 80%, 85%, 90%, 91%, 92%, 93%, 94%, 95%, 96%, 97%, 98%, or 99% identity to any one of the sequences of Table 10 or binding fragments thereof.

In some embodiments, the CD19 binding molecule comprises a heavy chain variable (VH) domain and a light chain variable (VL) domain. In some embodiments, the VH and VL domains are selected from an amino acid sequence comprising at least 80%, 85%, 90%, 91%, 92%, 93%, 94%, 95%, 96%, 97%, 98%, or 99% identity to any one of the sequences of Table 10, or fragments thereof. In some embodiments, the CD19 binding molecule comprises a heavy chain variable domain (VH), comprising a first complementary determining region of the heavy chain (VH CDR1), a second complementary determining region of the heavy chain (VH CDR2), and a third complementary determining region of the heavy chain (VH CDR3); and a light chain variable domain (VL), comprising a first complementary determining region of the light chain (VL CDR1), a second complementary determining region of the light chain (VL CDR2), and a third complementary determining region of the light chain (VL CDR3).

In some embodiments, the CD19 binding molecule comprises a heavy chain variable (VH) domain and a light chain variable (VL) domain, wherein the heavy chain variable domain comprises a VH CDR1 comprising SYVMH (SEQ ID NO: 406), a VH CDR2 comprising NPYNDG (SEQ ID NO: 407), a VH CDR3 comprising GTYYYGTRVFDY (SEQ ID NO: 408), and wherein the light chain variable domain comprises a VL CDR1 comprising RSSKSLQNVNGNTYLY (SEQ ID NO: 409), a VL CDR2 comprising RMSNLNS (SEQ ID NO: 410), and a VL CDR3 comprising MQHLEYPIT (SEQ ID NO: 411).

In some embodiments, the CD19 binding molecule comprises a heavy chain variable (VH) domain and a light chain variable (VL) domain, wherein the heavy chain variable domain comprises a VH CDR1 comprising TSGMGVG (SEQ ID NO: 418), a VH CDR2 comprising WWDDD (SEQ ID NO: 419), a VH CDR3 comprising MELWSYYFDY (SEQ ID NO: 420), and wherein the light chain variable domain comprises a VL CDR1 comprising SVSYMH (SEQ ID NO: 421), a VL CDR2 comprising DASNRAS (SEQ ID NO: 422), and a VL CDR3 comprising FQGSVYPFTF (SEQ ID NO: 423).

In some embodiments, the CD19 binding molecule comprises a heavy chain variable (VH) domain and a light chain variable (VL) domain, wherein the heavy chain variable domain comprises a VH CDR1 comprising KASGHTISSYAYS (SEQ ID NO: 424), a VH CDR2 comprising DIIPAYGSPN (SEQ ID NO: 425), a VH CDR3 comprising AREDFGKNYAMDV (SEQ ID NO: 426), and wherein the light chain variable domain comprises a VL CDR1 comprising RASQHVSSHYLA (SEQ ID NO: 427), a VL CDR2 comprising YGASSRAT (SEQ ID NO: 428), and a VL CDR3 comprising QHYGQSQFT (SEQ ID NO: 429).

In some embodiments, the CD19 binding molecule comprises a heavy chain variable (VH) domain and a light chain variable (VL) domain, wherein the heavy chain variable domain comprises a VH CDR1 comprising TSTMGVG (SEQ ID NO: 412), a VH CDR2 comprising FIWWDDDKRYNPNLKS (SEQ ID NO: 413), a VH CDR3 comprising MELWSYYFDY (SEQ ID NO: 414), and wherein the light chain variable domain comprises a VL CDR1 comprising SASSSVGYMH (SEQ ID NO: 415), a VL CDR2 comprising DTSKLAS (SEQ ID NO: 416), and a VL CDR3 comprising FQGSVYPFT (SEQ ID NO: 417).

In some embodiments, the CD19 binding molecule comprises a heavy chain variable (VH) domain and a light chain variable (VL) domain, wherein the heavy chain variable domain comprises a VH CDR1 comprising DYIMH (SEQ ID NO: 430), a VH CDR2 comprising YINPYNDGSKYTDKFQE (SEQ ID NO: 431), a VH CDR3 comprising GTYYYGPELFDY (SEQ ID NO: 432), and wherein the light chain variable domain comprises a VL CDR1 comprising KSSQSLETTTGTTYLN (SEQ ID NO: 433), a VL CDR2 comprising RASKRFS (SEQ ID NO: 434), and a VL CDR3 comprising LQLLEDPYT (SEQ ID NO: 435).

In some embodiments, the CD19 binding molecule comprises VH and VL domains each selected from an amino acid sequence comprising at least 80%, 85%, 90%, 91%, 92%, 93%, 94%, 95%, 96%, 97%, 98%, or 99% identity to any one of the sequences of Table 10. In some embodiments, the CD19 molecule comprises:
(a) a heavy chain comprising the amino acid sequence of SEQ ID NO: 343 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 344 or an amino acid sequence having at least 80%, 85%, 90%, 91%, 92%, 93%, 94%, 95%, 96%, 97%, 98%, or 99% identity therewith;
(b) a heavy chain comprising the amino acid sequence of SEQ ID NO: 345 or an amino acid sequence having at least 80%, 85%, 90%, 91%, 92%, 93%, 94%, 95%, 96%, 97%, 98%, or 99% identity therewith and a light chain comprising the amino acid sequence of SEQ ID NO: 346 or an amino acid sequence having at least 80%, 85%, 90%, 91%, 92%, 93%, 94%, 95%, 96%, 97%, 98%, or 99% identity therewith;
(c) a heavy chain comprising the amino acid sequence of SEQ ID NO: 347 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 348 or an amino acid sequence having at least 80%, 85%, 90%, 91%, 92%, 93%, 94%, 95%, 96%, 97%, 98%, or 99% identity therewith;
(d) a heavy chain comprising the amino acid sequence of SEQ ID NO: 349 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 350 or an amino acid sequence having at least 80%, 85%, 90%, 91%, 92%, 93%, 94%, 95%, 96%, 97%, 98%, or 99% identity therewith;
(e) a heavy chain comprising the amino acid sequence of SEQ ID NO: 351 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 352 or an amino acid sequence having at least 80%, 85%, 90%, 91%, 92%, 93%, 94%, 95%, 96%, 97%, 98%, or 99% identity therewith;
(f) a heavy chain comprising the amino acid sequence of SEQ ID NO: 353 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 354 or an amino acid sequence having at least 80%, 85%, 90%, 91%, 92%, 93%, 94%, 95%, 96%, 97%, 98%, or 99% identity therewith;
(g) a heavy chain comprising the amino acid sequence of SEQ ID NO: 355 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 356 or an amino acid sequence having at least 80%, 85%, 90%, 91%, 92%, 93%, 94%, 95%, 96%, 97%, 98%, or 99% identity therewith;
(h) a heavy chain comprising the amino acid sequence of SEQ ID NO: 357 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 358 or an amino acid sequence having at least 80%, 85%, 90%, 91%, 92%, 93%, 94%, 95%, 96%, 97%, 98%, or 99% identity therewith; or
(i) a heavy chain comprising the amino acid sequence of SEQ ID NO: 359 or an amino acid sequence having at least 80%, 85%, 90%, 91%, 92%, 93%, 94%, 95%, 96%, 97%, 98%, or 99% identity therewith, and a light chain comprising the amino acid sequence of SEQ ID NO: 348 or an amino acid sequence having at least 80%, 85%, 90%, 91%, 92%, 93%, 94%, 95%, 96%, 97%, 98%, or 99% identity therewith.
(j) a VH domain comprising the amino acid sequence of SEQ ID NO: 360 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 361 or an amino acid sequence having at least 80%, 85%, 90%, 91%, 92%, 93%, 94%, 95%, 96%, 97%, 98%, or 99% identity therewith;
(k) a VH domain comprising the amino acid sequence of SEQ ID NO: 362 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 363 or an amino acid sequence having at least 80%, 85%, 90%, 91%, 92%, 93%, 94%, 95%, 96%, 97%, 98%, or 99% identity therewith;
(l) a VH domain comprising the amino acid sequence of SEQ ID NO: 369 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 370 or an amino acid sequence having at least 80%, 85%, 90%, 91%, 92%, 93%, 94%, 95%, 96%, 97%, 98%, or 99% identity therewith;
(m)a VH domain comprising the amino acid sequence of SEQ ID NO: 371 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 372 or an amino acid sequence having at least 80%, 85%, 90%, 91%, 92%, 93%, 94%, 95%, 96%, 97%, 98%, or 99% identity therewith;
(n) a VH domain comprising the amino acid sequence of SEQ ID NO: 373 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 374 or an amino acid sequence having at least 80%, 85%, 90%, 91%, 92%, 93%, 94%, 95%, 96%, 97%, 98%, or 99% identity therewith;
(o) a VH domain comprising the amino acid sequence of SEQ ID NO: 375 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 376 or an amino acid sequence having at least 80%, 85%, 90%, 91%, 92%, 93%, 94%, 95%, 96%, 97%, 98%, or 99% identity therewith;
(p) a VH domain comprising the amino acid sequence of SEQ ID NO: 377 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 378 or an amino acid sequence having at least 80%, 85%, 90%, 91%, 92%, 93%, 94%, 95%, 96%, 97%, 98%, or 99% identity therewith;
(q) a VH domain comprising the amino acid sequence of SEQ ID NO: 379 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 380 or an amino acid sequence having at least 80%, 85%, 90%, 91%, 92%, 93%, 94%, 95%, 96%, 97%, 98%, or 99% identity therewith;
(r) a VH domain comprising the amino acid sequence of SEQ ID NO: 381 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 382 or an amino acid sequence having at least 80%, 85%, 90%, 91%, 92%, 93%, 94%, 95%, 96%, 97%, 98%, or 99% identity therewith;
(s) a VH domain comprising the amino acid sequence of SEQ ID NO: 383 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 384 or an amino acid sequence having at least 80%, 85%, 90%, 91%, 92%, 93%, 94%, 95%, 96%, 97%, 98%, or 99% identity therewith;
(t) a VH domain comprising the amino acid sequence of SEQ ID NO: 385 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 386 or an amino acid sequence having at least 80%, 85%, 90%, 91%, 92%, 93%, 94%, 95%, 96%, 97%, 98%, or 99% identity therewith; or
(u) a VH domain comprising the amino acid sequence of SEQ ID NO: 387 or an amino acid sequence having at least 80%, 85%, 90%, 91%, 92%, 93%, 94%, 95%, 96%, 97%, 98%, or 99% identity therewith, and a VL domain comprising the amino acid sequence of SEQ ID NO: 388 or an amino acid sequence having at least 80%, 85%, 90%, 91%, 92%, 93%, 94%, 95%, 96%, 97%, 98%, or 99% identity therewith.

In some embodiments, the CD19 binding molecule comprises at least 80%, 85%, 90%, 91%, 92%, 93%, 94%, 95%, 96%, 97%, 98%, or 99% identity to any one of the sequences of Table 10 or a fragment thereof (e.g., a VH, VL, or scFv thereof). In some embodiments, the CD19 binding molecule is an antibody or antigen binding fragment. In some embodiments the antibody or antigen binding fragment comprises at least 80%, 85%, 90%, 91%, 92%, 93%, 94%, 95%, 96%, 97%, 98%, or 99% identity to any one of the sequences of Table 10. In some embodiments, the CD19 binding molecule is a single domain antibody (sdAb), a camelid binding molecule, a VHH, a nanobody, a Fab fragment, a F(ab′)2 fragment, or an scFv

In some embodiments, the CD19 binding molecule is an scFv. In some embodiments, the scFv comprises at least 80%, 85%, 90%, 91%, 92%, 93%, 94%, 95%, 96%, 97%, 98%, or 99% identity to any one of the sequences of Table 10.

In some embodiments the CD19 binding molecule is a chimeric antigen receptor (CAR). In some embodiments, the CAR comprises at least 80%, 85%, 90%, 91%, 92%, 93%, 94%, 95%, 96%, 97%, 98%, or 99% identity to any one of the sequences of Table 10. Exemplary CARs are described elsewhere herein. In some embodiments, said CD19 binding molecule further comprises a CD28z costimulatory domain as described herein and optionally exhibits increased activity compared to a suitable control having an alternate costimulatory domain. In some embodiments, said CD19 binding molecule further comprises a 4-1BB costimulatory domain as described herein and optionally exhibits increased activity compared to a suitable control having an alternate costimulatory domain.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 4, an IRES from a construct of Table 5, and an expression sequence encoding a CD19 binding molecule comprising at least 85%, 86%, 87%, 88%, 89%, 90%, 91%, 92%, 93%, 94%, 95%, 96%, 97%, 98%, 99%, or 100% sequence identity to any one of the sequences of Table 9 or Table 10 or a fragment thereof.

In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 4 and an expression sequence encoding a polypeptide CD19 binding molecule comprising a heavy chain variable (VH) domain and a light chain variable (VL) domain described above. In some embodiments, the circular RNA constructs and related pharmaceutical compositions disclosed herein comprise an IRES sequence having at least 85%, 86%, 87%, 88%, 89%, 90%, 91%, 92%, 93%, 94%, 95%, 96%, 97%, 98%, 99%, or 100% sequence identity to an IRES from Table 4, and an expression sequence encoding a polypeptide CD19 binding molecule comprising at least 85%, 86%, 87%, 88%, 89%, 90%, 91%, 92%, 93%, 94%, 95%, 96%, 97%, 98%, 99%, or 100% sequence identity to any one of the sequences of Table 10, or a fragment thereof.

As a non-limiting example, in some embodiments, the circular RNA constructs and related pharmaceutical compositions disclosed herein comprise an expression sequence encoding a CD19 binding molecule that comprises at least 80%, 85%, 90%, 91%, 92%, 93%, 94%, 95%, 96%, 97%, 98%, or 99% identity to a sequence of Table 10 or a fragment thereof (e.g., a VH, VL, scFv or CAR sequence of Table 10, e.g., 10-10), and an IRES sequence having at least 85%, 86%, 87%, 88%, 89%, 90%, 91%, 92%, 93%, 94%, 95%, 96%, 97%, 98%, 99%, or 100% sequence identity to an IRES from Table 4 (e.g., 4-19 or 4-21).

TABLE 9
Additional Codon Amino Acid and Nucleotide Sequences

| | | Codon amino acid |
|---|---|---|
| ID | Codon nucleotide sequence | sequence |
| 9-1 | ATGGGCGTGCCTACCCAGCTGCTCGGTCTCCTGC | MGVPTQLLGLLL |
| | TGCTCTGGATCACCGACGCTATCTGCGACATCCA | LWITDAICDIQM |
| | AATGACCCAGAGTCCCGCTTCCCTCAGCACCTCC | TQSPASLSTSLGE |
| | CTGGGTGAGACCGTCACCATCCAGTGCCAGGCA | TVTIQCQASEDIY |
| | TCCGAGGACATCTACAGTGGTCTCGCCTGGTACC | SGLAWYQQKPG |
| | AGCAGAAGCCTGGTAAGTCCCCTCAGCTGCTGA | KSPQLLIYGASD |
| | TCTACGGTGCTTCCGATCTGCAGGACGGAGTCCC | LQDGVPSRFSGS |
| | TAGCCGCTTCTCAGGCTCTGGCTCCGGTACCCAG | GSGTQYSLKITS |
| | TACTCCCTGAAGATCACATCCATGCAGACCGAA | MQTEDEGVYFC |
| | GACGAGGGAGTGTACTTCTGCCAGCAGGGGCTG | QQGLTYPRTFGG |
| | ACCTACCCTCGGACATTCGGCGGTGGAACCAAG | GTKLELKGGGGS |
| | CTCGAGCTGAAGGGAGGTGGAGGCAGTGGTGGC | GGGGSGGGGSE |
| | GGAGGATCTGGTGGTGGTGGCTCCGAGGTCCAA | VQLQQSGAELV |
| | CTGCAGCAGTCCGGCGCTGAGCTGGTGAGGCCC | RPGTSVKLSCKV |
| | GGAACCAGCGTCAAACTCAGCTGCAAGGTGAGC | SGDTITFYYMHF |
| | GGGGACACCATCACCTTCTACTACATGCACTTCG | VKQRPGQGLEWI |
| | TCAAGCAGAGGCCTGGGCAGGGTCTTGAATGGA | GRIDPEDESTKY |
| | TCGGCCGGATCGATCCAGAGGACGAGTCTACAA | SEKFKNKATLTA |
| | AGTACTCCGAGAAGTTCAAGAACAAAGCAACCC | DTSSNTAYLKLS |
| | TGACCGCCGACACAAGCTCCAACACCGCCTACC | SLTSEDTATYFCI |
| | TGAAGCTGTCCAGCCTCACCTCTGAGGACACCG | YGGYYFDYWGQ |
| | CCACCTACTTCTGCATCTACGGCGGGTACTACTT | GVMVTVSSIEFM |
| | CGACTATTGGGGCCAAGGGGTGATGGTCACCGT | YPPPYLDNERSN |
| | GTCCTCTATCGAGTTCATGTATCCTCCTCCCTAC | GTIIHIKEKHLCH |
| | CTGGACAACGAGCGGAGCAACGGCACCATCATC | TQSSPKLFWALV |
| | CACATCAAAGAGAAGCACCTCTGCCACACCCAA | VVAGVLFCYGL |
| | TCCTCTCCCAAACTCTTCTGGGCCCTCGTTGTGG | LVTVALCVIWTN |
| | TCGCAGGCGTGCTCTTCTGCTACGGGCTGCTGGT | SRRNRGGQSDY |
| | GACTGTGGCCTTGTGCGTGATCTGGACCAACAG | MNMTPRRPGLT |
| | TAGACGGAATCGGGGAGGTCAGAGCGACTACAT | RKPYQPYAPARD |
| | GAACATGACACCTCGCAGACCAGGCCTGACACG | FAAYRPRAKFSR |
| | GAAGCCCTACCAACCATACGCTCCTGCCCGGGA | SAETAANLQDPN |
| | TTTTGCAGCATATCGGCCACGGGCCAAGTTTAGC | QLYNELNLGRRE |
| | AGGTCCGCAGAGACCGCAGCCAACCTGCAAGAC | EYDVLEKKRAR |
| | CCTAACCAGCTGTACAACGAGCTGAACCTTGGT | DPEMGGKQQRR |
| | CGCCGGGAGGAGTACGACGTCCTGGAGAAGAA | RNPQEGVYNAL |
| | GAGAGCACGGGATCCCGAGATGGGCGGAAAGC | QKDKMAEAYSEI |
| | AACAACGCCGGCGGAATCCTCAGGAGGGTGTCT | GTKGERRRGKG |
| | ACAACGCCCTCCAGAAGGACAAGATGGCTGAGG | HDGLYQGLSTAT |
| | CCTACTCCGAGATCGGCACTAAGGGCGAGCGCA | KDTYDALHMQT |
| | GACGGGGAAAGGGTCACGACGGGCTGTACCAG | LAPR (SEQ ID |
| | GGTCTCAGCACCGCAACCAAGGATACCTACGAC | NO: 327) |
| | GCCCTGCACATGCAAACCCTCGCACCCCGG (SEQ | |
| | ID NO: 311) | |
| 9-2 | ATGGCTCTGCCTGTGACAGCTCTGCTGCTGCCTC | MALPVTALLLPL |
| | TGGCTCTGCTTCTGCATGCCGCCAGACCTGACAT | ALLLHAARPDIQ |
| | CCAGATGACTCAGAGCCCCAGCAGCCTGTCTGC | MTQSPSSLSASV |
| | CTCTGTGGGAGACAGAGTGACAATTACCTGCCG | GDRVTITCRASQ |
| | GGCCAGCCAGGATGTGAATACTGCTGTCGCCTG | DVNTAVAWYQQ |
| | GTATCAACAAAAGCCTGGCAAGGCCCCTAAGCT | KPGKAPKLLIYS |
| | CCTGATCTACAGCGCCAGCTTTCTGTACAGCGGC | ASFLYSGVPSRFS |
| | GTGCCCAGCAGATTCTCCGGAAGCAGAAGCGGC | GSRSGTDFTLTIS |
| | ACAGATTTCACACTGACCATAAGCAGCCTGCAG | SLQPEDFATYYC |
| | CCAGAGGATTTCGCCACCTACTATTGCCAGCAG | QQHYTTPPTFGQ |
| | CACTACACCACACCTCCAACCTTTGGCCAGGGC | GTKVEIKRTGST |
| | ACCAAGGTCGAGATTAAGAGAACAGGCAGCAC | SGSGKPGSGEGS |
| | ATCTGGCTCTGGCAAACCTGGATCTGGCGAGGG | EVQLVESGGGLV |
| | CTCTGAAGTCCAGCTGGTGGAATCTGGCGGAGG | QPGGSLRLSCAA |
| | ACTGGTTCAACCTGGCGGCTCTCTGAGACTGTCT | SGFNIKDTYIHW |
| | TGTGCCGCCTCCGGCTTCAACATCAAGGACACCT | VRQAPGKGLEW |
| | ACATCCACTGGGTCCGACAAGCCCCAGGCAAAG | VARIYPTNGYTR |
| | GACTTGAGTGGGTCGCCAGGATCTACCCCACCA | YADSVKGRFTIS |
| | ACGGCTACACCAGATACGCCGACTCTGTGAAGG | ADTSKNTAYLQ |
| | GCAGATTCACCATCTCTGCCGACACCAGCAAGA | MNSLRAEDTAV |
| | ATACCGCCTACCTGCAGATGAACTCCCTGAGAG | YYCSRWGGDGF |
| | CCGAAGATACCGCTGTGTATTACTGTTCCAGATG | YAMDVWGQGT |
| | GGGAGGCGACGGCTTCTACGCCATGGATGTTTG | LVTVSSIEVMYP |
| | GGGCCAAGGCACCCTCGTGACCGTTTCTTCTATC | PPYLDNEKSNGT |
| | GAAGTGATGTACCCTCCACCTTACCTGGACAAC | IIHVKGKHLCPSP |
| | GAGAAGTCCAACGGCACCATCATCCACGTGAAG | LFPGPSKPFWVL |
| | GGCAAGCACCTGTGTCCTTCTCCACTGTTCCCCG | VVVGGVLACYS |
| | GACCTAGCAAGCCTTTCTGGGTGCTCGTTGTTGT | LLVTVAFIIFWV |
| | TGGCGGCGTGCTGGCCTGTTACTCTCTGCTGGTT | RSKRSRLLHSDY |
| | ACCGTGGCCTTCATCATCTTTTGGGTCCGAAGCA | MNMTPRRPGPT |
| | AGCGGAGCCGGCTGCTGCACTCCGACTACATGA | RKHYQPYAPPRD |
| | ACATGACCCCTAGACGGCCCGGACCAACCAGAA | FAAYRSRVKFSR |
| | AGCACTACCAGCCTTACGCTCCTCCTAGAGACTT | SADAPAYQQGQ |
| | CGCCGCCTACCGGTCCAGAGTGAAGTTCAGCAG | NQLYNELNLGR |
| | ATCCGCCGATGCTCCCGCCTATCAGCAGGGCCA | REEYDVLDKRR |
| | AAACCAGCTGTACAACGAGCTGAACCTGGGGAG | GRDPEMGGKPR |
| | AAGAGAAGAGTACGACGTGCTGGACAAGCGGA | RKNPQEGLYNEL |
| | GAGGCAGAGATCCTGAAATGGGCGGCAAGCCCA | QKDKMAEAYSEI |
| | GACGGAAGAATCCTCAAGAGGGCCTGTATAATG | GMKGERRRGKG |
| | AGCTGCAGAAAGACAAGATGGCCGAGGCCTACA | HDGLYQGLSTAT |
| | GCGAGATCGGAATGAAGGGCGAGCGCAGAAGA | KDTYDALHMQA |
| | GGCAAGGGACACGATGGACTGTACCAGGGCCTG | LPPR (SEQ ID |
| | AGCACCGCCACCAAGGATACCTATGATGCCCTG | NO: 285) |
| | CACATGCAGGCCCTGCCTCCAAGA (SEQ ID NO: | |
| | 249) | |
| 9-3 | ATGGCTCTGCCTGTGACAGCTCTGCTGCTGCCTC | MALPVTALLLPL |
| | TGGCTCTGCTTCTGCATGCCGCCAGACCTGACAT | ALLLHAARPDIQ |
| | CCAGATGACTCAGAGCCCCAGCAGCCTGTCTGC | MTQSPSSLSASV |
| | CTCTGTGGGAGACAGAGTGACAATTACCTGCCG | GDRVTITCRASQ |
| | GGCCAGCCAGGATGTGAATACTGCTGTCGCCTG | DVNTAVAWYQQ |
| | GTATCAACAAAAGCCTGGCAAGGCCCCTAAGCT | KPGKAPKLLIYS |
| | CCTGATCTACAGCGCCAGCTTTCTGTACAGCGGC | ASFLYSGVPSRFS |
| | GTGCCCAGCAGATTCTCCGGAAGCAGAAGCGGC | GSRSGTDFTLTIS |
| | ACAGATTTCACACTGACCATAAGCAGCCTGCAG | SLQPEDFATYYC |
| | CCAGAGGATTTCGCCACCTACTATTGCCAGCAG | QQHYTTPPTFGQ |
| | CACTACACCACACCTCCAACCTTTGGCCAGGGC | GTKVEIKRTGST |
| | ACCAAGGTCGAGATTAAGAGAACAGGCAGCAC | SGSGKPGSGEGS |
| | ATCTGGCTCTGGCAAACCTGGATCTGGCGAGGG | EVQLVESGGGLV |
| | CTCTGAAGTCCAGCTGGTGGAATCTGGCGGAGG | QPGGSLRLSCAA |
| | ACTGGTTCAACCTGGCGGCTCTCTGAGACTGTCT | SGFNIKDTYIHW |
| | TGTGCCGCCTCCGGCTTCAACATCAAGGACACCT | VRQAPGKGLEW |
| | ACATCCACTGGGTCCGACAAGCCCCAGGCAAAG | VARIYPTNGYTR |
| | GACTTGAGTGGGTCGCCAGGATCTACCCCACCA | YADSVKGRFTIS |
| | ACGGCTACACCAGATACGCCGACTCTGTGAAGG | ADTSKNTAYLQ |
| | GCAGATTCACCATCTCTGCCGACACCAGCAAGA | MNSLRAEDTAV |
| | ATACCGCCTACCTGCAGATGAACTCCCTGAGAG | YYCSRWGGDGF |
| | CCGAAGATACCGCTGTGTATTACTGTTCCAGATG | YAMDVWGQGT |
| | GGGAGGCGACGGCTTCTACGCCATGGATGTTTG | LVTVSSTTTPAP |
| | GGGCCAAGGCACCCTCGTGACCGTTTCTTCTACC | RPPTPAPTIASQP |
| | ACCACACCAGCTCCTCGGCCTCCAACTCCTGCTC | LSLRPEACRPAA |
| | CTACAATTGCCAGCCAGCCTCTGTCTCTGAGGCC | GGAVHTRGLDF |
| | CGAAGCTTGTAGACCTGCTGCTGGCGGAGCCGT | ACDIYIWAPLAG |
| | GCATACAAGAGGACTGGATTTCGCCTGCGACAT | TCGVLLLSLVITL |
| | CTACATCTGGGCTCCTCTGGCCGGAACATGTGGC | YCKRGRKKLLYI |
| | GTTCTGCTGCTGAGCCTGGTCATCACCCTGTACT | FKQPFMRPVQTT |
| | GTAAGCGGGGCAGAAAGAAGCTGCTGTACATCT | QEEDGCSCRFPE |
| | TCAAGCAGCCCTTCATGCGGCCCGTGCAGACCA | EEEGGCELRVKF |
| | CACAAGAGGAAGATGGCTGCTCCTGCAGATTCC | SRSADAPAYQQ |
| | CCGAGGAAGAAGAAGGCGGCTGCGAGCTGAGA | GQNQLYNELNL |
| | GTGAAGTTCAGCAGATCCGCCGATGCTCCCGCC | GRREEYDVLDK |
| | TATCAGCAGGGCCAAAACCAGCTGTACAACGAG | RRGRDPEMGGK |
| | CTGAACCTGGGGAGAAGAGAAGAGTACGACGT | PRRKNPQEGLYN |
| | GCTGGACAAGCGGAGAGGCAGAGATCCTGAAAT | ELQKDKMAEAY |
| | GGGCGGCAAGCCCAGACGGAAGAATCCTCAAG | SEIGMKGERRRG |
| | AGGGCCTGTATAATGAGCTGCAGAAAGACAAGA | KGHDGLYQGLS |
| | TGGCCGAGGCCTACAGCGAGATCGGAATGAAGG | TATKDTYDALH |
| | GCGAGCGCAGAAGAGGCAAGGGACACGATGGA | MQALPPR (SEQ |
| | CTGTACCAGGGCCTGAGCACCGCCACCAAGGAT | ID NO: 286) |
| | ACCTATGATGCCCTGCACATGCAGGCCCTGCCTC | |
| | CAAGA (SEQ ID NO: 250) | |
| 9-4 | ATGGCACTCCCGGTAACCGCCTTATTGCTTCCCC | MALPVTALLLPL |
| | TTGCCCTCTTGCTCCACGCAGCACGCCCCGATAT | ALLLHAARPDIV |
| | AGTCTTGACTCAATCCCCACCCAGTTTGGCAATG | LTQSPPSLAMSL |
| | TCATTAGGCAAACGAGCAACAATTTCATGTAGG | GKRATISCRASE |
| | GCATCCGAAAGTGTAACGATTTTGGGGAGTCAT | SVTILGSHLIHW |
| | TTAATTCATTGGTACCAACAAAAGCCTGGACAA | YQQKPGQPPTLL |
| | CCCCCGACGCTCTTGATCCAATTAGCATCTAACG | IQLASNVQTGVP |
| | TCCAAACCGGAGTCCCCGCACGATTCTCAGGAT | ARFSGSGSRTDF |
| | CCGGTTCCCGGACTGATTTTACATTAACTATTGA | TLTIDPVEEDDV |
| | TCCGGTAGAGGAAGATGACGTCGCTGTCTATTA | AVYYCLQSRTIP |
| | TTGTCTTCAAAGTAGGACGATTCCACGGACATTC | RTFGGGTKLEIK |
| | GGTGGCGGAACTAAATTGGAGATTAAAGGTTCC | GSTSGSGKPGSG |
| | ACCTCTGGTAGTGGGAAACCCGGGTCCGGTGAA | EGSTKGQIQLVQ |
| | GGGTCCACTAAAGGCCAAATTCAACTCGTTCAA | SGPELKKPGETV |
| | TCCGGACCAGAACTGAAGAAGCCAGGAGAAACT | KISCKASGYTFT |
| | GTCAAAATAAGCTGTAAAGCTTCCGGTTATACA | DYSINWVKRAP |
| | TTTACAGATTATTCCATAAATTGGGTGAAAAGG | GKGLKWMGWIN |
| | GCGCCAGGAAAAGGGTTAAAGTGGATGGGTTGG | TETREPAYAYDF |
| | ATTAATACAGAGACTCGGGAACCTGCATATGCT | RGRFAFSLETSA |
| | TATGATTTTAGGGGAAGGTTTGCCTTTTCTCTGG | STAYLQINNLKY |
| | AGACTTCCGCTTCAACTGCTTATCTCCAAATTAA | EDTATYFCALDY |
| | TAATCTTAAATATGAGGACACAGCAACATACTT | SYAMDYWGQGT |
| | CTGTGCTTTGGACTATAGTTATGCTATGGATTAC | SVTVSSAAATTT |
| | TGGGGACAAGGAACCAGTGTCACTGTAAGTTCC | PAPRPPTPAPTIA |
| | GCTGCTGCGACGACCACTCCTGCACCGCGACCA | SQPLSLRPEACRP |
| | CCCACTCCTGCCCCTACTATTGCTAGTCAACCAC | AAGGAVHTRGL |
| | TTAGCTTGCGACCTGAGGCATGTCGGCCCGCGG | DFACDIYIWAPL |
| | CAGGTGGCGCAGTCCACACCAGGGGTTTAGACT | AGTCGVLLLSLV |
| | TTGCTTGTGATATTTATATTTGGGCACCACTCGC | ITLYCKRGRKKL |
| | CGGGACTTGCGGTGTTCTTCTCTTGTCCCTTGTT | LYIFKQPFMRPV |
| | ATAACTCTTTATTGTAAGCGCGGAAGGAAGAAA | QTTQEEDGCSCR |
| | TTGTTATATATTTTCAAACAACCTTTTATGCGAC | FPEEEEGGCELR |
| | CCGTACAAACAACTCAGGAAGAGGACGGGTGTT | VKFSRSADAPAY |
| | CTTGTCGGTTTCCAGAAGAGGAAGAGGGTGGGT | QQGQNQLYNEL |
| | GTGAACTCCGGGTCAAATTTAGTAGGTCAGCAG | NLGRREEYDVL |
| | ATGCGCCGGCGTACCAACAAGGCCAAAACCAAC | DKRRGRDPEMG |
| | TGTATAATGAACTCAATCTCGGTAGGCGTGAGG | GKPRRKNPQEGL |
| | AATATGATGTCCTTGATAAAAGGCGCGGGAGAG | YNELQKDKMAE |
| | ATCCAGAAATGGGCGGAAAACCACGGCGAAAG | AYSEIGMKGERR |
| | AATCCGCAGGAAGGGTTATATAACGAACTTCAA | RGKGHDGLYQG |
| | AAGGATAAAATGGCTGAAGCTTATTCCGAAATT | LSTATKDTYDAL |
| | GGCATGAAAGGAGAGCGACGTAGGGGCAAAGG | HMQALPPR (SEQ |
| | GCATGATGGCCTTTACCAAGGGCTCTCAACCGCT | ID NO: 279) |
| | ACAAAAGATACTTACGACGCTTTACATATGCAA | |
| | GCACTTCCACCCAGG (SEQ ID NO: 243) | |
| 9-5 | ATGGCCCTCCCCGTCACAGCTCTCCTGCTCCCAC | MALPVTALLLPL |
| | TGGCCCTTCTTTTGCACGCTGCTCGCCCCGATAT | ALLLHAARPDIV |
| | CGTGCTCACCCAGTCACCTCCAAGCCTTGCCATG | LTQSPPSLAMSL |
| | AGCCTCGGGAAACGGGCTACCATCTCCTGCCGG | GKRATISCRASE |
| | GCTTCAGAGTCCGTCACCATCCTCGGGTCACACC | SVTILGSHLIHW |
| | TCATCCACTGGTACCAACAGAAACCAGGGCAGC | YQQKPGQPPTLL |
| | CTCCTACCCTCTTGATCCAGTTGGCCTCCAACGT | IQLASNVQTGVP |
| | GCAAACTGGGGTTCCCGCCAGGTTCAGTGGCTC | ARFSGSGSRTDF |
| | CGGATCCCGGACAGATTTCACACTTACCATCGAT | TLTIDPVEEDDV |
| | CCTGTGGAGGAGGACGATGTGGCCGTCTATTAC | AVYYCLQSRTIP |
| | TGCCTGCAGTCTCGCACCATCCCTCGGACCTTCG | RTFGGGTKLEIK |
| | GTGGAGGCACCAAGCTCGAGATCAAGGGTAGCA | GSTSGSGKPGSG |
| | CCTCCGGCTCTGGAAAGCCAGGCTCTGGTGAGG | EGSTKGQIQLVQ |
| | GTTCTACCAAGGGCCAAATCCAGCTGGTCCAGT | SGPELKKPGETV |
| | CTGGGCCCGAGCTGAAGAAACCCGGGGAGACCG | KISCKASGYTFT |
| | TGAAGATCTCCTGCAAGGCCTCCGGTTATACCTT | DYSINWVKRAP |
| | CACCGACTACTCCATCAACTGGGTCAAGCGCGC | GKGLKWMGWIN |
| | TCCTGGAAAGGGCCTCAAGTGGATGGGCTGGAT | TETREPAYAYDF |
| | CAACACCGAAACCCGCGAGCCTGCCTATGCTTA | RGRFAFSLETSA |
| | CGACTTCAGGGGCCGGTTCGCTTTCTCACTGGAG | STAYLQINNLKY |
| | ACCTCCGCTTCCACAGCCTACCTCCAGATCAACA | EDTATYFCALDY |
| | ACCTCAAGTACGAAGACACCGCCACCTATTTCT | SYAMDYWGQGT |
| | GCGCTCTCGACTATTCCTACGCTATGGACTACTG | SVTVSSAAATTT |
| | GGGTCAGGGCACCTCTGTGACCGTCTCTAGCGC | PAPRPPTPAPTIA |
| | AGCCGCCACCACAACACCAGCCCCACGGCCACC | SQPLSLRPEACRP |
| | TACTCCCGCACCCACCATCGCATCCCAACCACTC | AAGGAVHTRGL |
| | AGTCTGAGGCCCGAGGCCTGTAGACCTGCTGCT | DFACDIYIWAPL |
| | GGAGGCGCAGTGCATACCCGCGGTCTCGACTTC | AGTCGVLLLSLV |
| | GCCTGCGACATCTATATCTGGGCCCCATTGGCAG | ITLYCRSKRSRLL |
| | GTACCTGTGGCGTGCTGCTGCTGTCACTCGTCAT | HSDYMNMTPRR |
| | CACCCTGTACTGCCGGAGTAAGCGCTCTAGGCT | PGPTRKHYQPYA |
| | GTTGCACAGCGACTACATGAACATGACCCCAAG | PPRDFAAYRSRV |
| | AAGACCAGGGCCTACCCGGAAGCACTACCAGCC | KFSRSADAPAYQ |
| | ATACGCACCTCCCCGGGACTTTGCCGCCTATCGG | QGQNQLYNELN |
| | TCTCGGGTGAAGTTCTCACGCTCCGCTGATGCCC | LGRREEYDVLD |
| | CAGCATACCAGCAGGGGCAGAACCAGCTGTACA | KRRGRDPEMGG |
| | ATGAGCTCAACCTCGGTCGCCGCGAAGAGTACG | KPRRKNPQEGLY |
| | ACGTGCTCGACAAGAGAAGGGGCAGGGACCCTG | NELQKDKMAEA |
| | AGATGGGAGGCAAGCCCCGCAGAAAGAATCCCC | YSEIGMKGERRR |
| | AGGAAGGTCTGTACAACGAGCTGCAAAAGGATA | GKGHDGLYQGL |
| | AGATGGCTGAGGCCTACAGCGAGATCGGCATGA | STATKDTYDALH |
| | AGGGCGAAAGGAGACGGGGAAAGGGCCACGAC | MQALPPR (SEQ |
| | GGGCTCTACCAGGGACTCTCCACCGCCACCAAG | ID NO: 281) |
| | GACACCTACGACGCCCTCCACATGCAGGCTCTG | |
| | CCACCCAGG (SEQ ID NO: 245) | |
| 9-6 | ATGGCACTTCCCGTCACCGCTCTCCTGCTGCCCC | MALPVTALLLPL |
| | TCGCACTGCTGCTCCATGCAGCCCGCCCAGACAT | ALLLHAARPDIV |
| | CGTCCTGACCCAGTCCCCTCCCTCCCTCGCAATG | LTQSPPSLAMSL |
| | TCCCTCGGGAAACGGGCCACCATCAGCTGCCGG | GKRATISCRASE |
| | GCCTCTGAGTCAGTGACAATCCTCGGAAGCCAT | SVTILGSHLIHW |
| | CTGATCCATTGGTACCAGCAGAAACCCGGTCAG | YQQKPGQPPTLL |
| | CCTCCAACCCTCCTCATCCAGCTGGCCTCCAACG | IQLASNVQTGVP |
| | TGCAGACAGGAGTCCCCGCTCGGTTCTCAGGCA | ARFSGSGSRTDF |
| | GCGGTTCCAGGACCGACTTCACCCTGACCATCG | TLTIDPVEEDDV |
| | ACCCCGTGGAAGAGGACGATGTGGCTGTGTACT | AVYYCLQSRTIP |
| | ACTGCCTCCAGTCCCGGACCATCCCACGGACCTT | RTFGGGTKLEIK |
| | CGGAGGTGGGACAAAGCTGGAGATCAAAGGCA | GSTSGSGKPGSG |
| | GCACCAGCGGTTCTGGCAAGCCAGGGTCAGGTG | EGSTKGQIQLVQ |
| | AGGGGAGCACAAAGGGTCAGATCCAGCTGGTGC | SGPELKKPGETV |
| | AGAGCGGTCCCGAGCTGAAGAAGCCCGGGGAG | KISCKASGYTFT |
| | ACCGTTAAGATCTCCTGCAAGGCTAGCGGGTAC | DYSINWVKRAP |
| | ACCTTCACCGACTATAGTATCAACTGGGTCAAG | GKGLKWMGWIN |
| | CGCGCTCCTGGCAAGGGGCTCAAGTGGATGGGG | TETREPAYAYDF |
| | TGGATCAACACCGAAACCAGGGAGCCCGCATAC | RGRFAFSLETSA |
| | GCTTATGACTTTCGGGGCCGGTTCGCCTTTTCCC | STAYLQINNLKY |
| | TGGAGACCAGCGCCTCTACCGCCTACCTCCAGA | EDTATYFCALDY |
| | TCAACAACCTGAAGTACGAGGACACCGCCACCT | SYAMDYWGQGT |
| | ACTTCTGCGCACTCGACTACTCCTACGCTATGGA | SVTVSSIEVMYP |
| | CTACTGGGGTCAGGGTACCTCCGTCACCGTCTCC | PPYLDNEKSNGT |
| | AGCATCGAGGTCATGTACCCTCCTCCCTACCTGG | IIHVKGKHLCPSP |
| | ACAACGAGAAGTCCAACGGCACCATCATCCATG | LFPGPSKPFWVL |
| | TGAAGGGCAAGCATCTCTGCCCCAGCCCACTGT | VVVGGVLACYS |
| | TCCCCGGACCCTCTAAGCCCTTCTGGGTCCTGGT | LLVTVAFIIFWV |
| | CGTCGTCGGCGGTGTTCTGGCTTGCTACAGCTTG | RSKRSRLLHSDY |
| | CTGGTCACCGTCGCCTTCATCATCTTCTGGGTGC | MNMTPRRPGPT |
| | GCTCCAAGAGGAGCCGGCTGCTGCATAGCGACT | RKHYQPYAPPRD |
| | ACATGAACATGACCCCTAGAAGGCCTGGTCCAA | FAAYRSRVKFSR |
| | CCCGCAAGCACTACCAGCCTTACGCCCCTCCAC | SADAPAYQQGQ |
| | GGGACTTCGCAGCCTACCGGTCACGGGTGAAGT | NQLYNELNLGR |
| | TCTCTCGGAGCGCAGATGCCCCAGCATACCAGC | REEYDVLDKRR |
| | AGGGCCAGAACCAGCTGTACAACGAACTTAACC | GRDPEMGGKPR |
| | TTGGTCGGCGGGAGGAATACGATGTGCTGGACA | RKNPQEGLYNEL |
| | AGCGCAGGGGTCGGGATCCTGAAATGGGCGGGA | QKDKMAEAYSEI |
| | AACCACGCCGGAAGAACCCACAGGAGGGGCTCT | GMKGERRRGKG |
| | ATAACGAGCTCCAAAAGGATAAGATGGCTGAGG | HDGLYQGLSTAT |
| | CTTACAGCGAGATTGGAATGAAGGGAGAAAGA | KDTYDALHMQA |
| | AGACGGGGCAAGGGTCACGACGGGTTGTACCAG | LPPR (SEQ ID |
| | GGTCTGAGCACCGCCACCAAGGACACCTACGAC | NO: 282) |
| | GCCCTCCACATGCAAGCCCTTCCACCCCGC (SEQ | |
| | ID NO: 246) | |
| 9-7 | ATGGCTCTCCCCGTGACCGCTCTGCTGCTCCCTC | MALPVTALLLPL |
| | TGGCCCTCCTTCTGCACGCAGCCAGACCACAGG | ALLLHAARPQV |
| | TCAAGCTGGAGGAGTCTGGTGGCGGTCTGGTGC | KLEESGGGLVQA |
| | AGGCAGGGAGGAGCCTGAGGCTGAGCTGTGCAG | GRSLRLSCAASE |
| | CTTCCGAGCACACATTCTCAAGCCACGTCATGG | HTFSSHVMGWF |
| | GGTGGTTCAGACAGGCTCCCGGTAAAGAGAGGG | RQAPGKERESVA |
| | AGTCCGTCGCCGTGATCGGATGGCGGGACATCT | VIGWRDISTSYA |
| | CCACCTCCTACGCCGACTCTGTGAAGGGCCGGTT | DSVKGRFTISRD |
| | CACAATCTCACGCGATAATGCCAAGAAGACACT | NAKKTLYLQMN |
| | GTATCTGCAGATGAATTCCTTGAAGCCCGAAGA | SLKPEDTAVYYC |
| | CACCGCCGTCTATTACTGTGCTGCTAGACGGATC | AARRIDAADFDS |
| | GACGCTGCCGACTTCGACAGCTGGGGACAGGGT | WGQGTQVTVSS |
| | ACCCAAGTGACCGTTTCCTCCGGAGGCGGAGGT | GGGGSGGGGSG |
| | TCTGGAGGAGGTGGGTCAGGTGGAGGTGGCTCC | GGGSEVQLVESG |
| | GAGGTGCAGCTGGTCGAGTCTGGCGGTGGCTTG | GGLVQAGGSLR |
| | GTCCAGGCTGGAGGCAGTCTCAGACTCTCCTGC | LSCAASGRTFTM |
| | GCTGCTTCAGGGCGGACCTTCACCATGGGCTGG | GWFRQAPGKER |
| | TTCAGGCAGGCCCCAGGTAAGGAGAGGGAGTTC | EFVAAISLSPTLA |
| | GTGGCCGCCATCTCCCTCTCCCCTACCCTGGCAT | YYAESVKGRFTI |
| | ACTACGCTGAGTCCGTGAAGGGACGGTTTACCA | SRDNAKNTVVL |
| | TCTCCCGGGATAACGCAAAGAACACTGTGGTCC | QMNSLKPEDTAL |
| | TCCAAATGAACTCCCTCAAACCCGAGGACACCG | YYCAADRKSVM |
| | CTCTCTACTATTGTGCCGCAGATCGGAAGAGCGT | SIRPDYWGQGTQ |
| | CATGTCCATCCGGCCCGATTACTGGGGCCAAGG | VTVSSTSTTTPAP |
| | CACACAGGTGACTGTGTCCAGCACCTCCACCAC | RPPTPAPTIASQP |
| | CACCCCAGCACCAAGGCCTCCAACCCCTGCACC | LSLRPEACRPAA |
| | AACCATCGCCTCCCAGCCACTGTCTTTGCGGCCA | GGAVHTRGLDF |
| | GAAGCATGCCGCCCAGCAGCAGGTGGAGCCGTG | ACDIYIWAPLAG |
| | CATACAAGAGGCCTGGACTTCGCCTGCGATATC | TCGVLLLSLVITL |
| | TACATCTGGGCTCCTCTGGCCGGAACATGCGGA | YCKRGRKKLLYI |
| | GTCCTGCTCTTGTCCCTGGTGATCACCCTGTACT | FKQPFMRPVQTT |
| | GCAAGCGGGGTCGGAAGAAGCTCCTCTACATCT | QEEDGCSCRFPE |
| | TCAAGCAGCCCTTCATGAGACCCGTCCAGACCA | EEEGGCELRVKF |
| | CCCAGGAGGAGGACGGGTGCTCATGCAGGTTCC | SRSADAPAYQQ |
| | CCGAAGAGGAGGAGGGTGGCTGTGAGCTGCGG | GQNQLYNELNL |
| | GTGAAGTTCAGCAGGTCAGCAGACGCCCCTGCC | GRREEYDVLDK |
| | TATCAGCAGGGCCAAAACCAGTTGTACAACGAG | RRGRDPEMGGK |
| | CTGAATCTGGGGAGACGGGAGGAGTACGATGTC | PRRKNPQEGLYN |
| | CTTGACAAGAGAAGGGGCCGGGATCCAGAGATG | ELQKDKMAEAY |
| | GGCGGGAAGCCAAGACGGAAGAATCCTCAGGA | SEIGMKGERRRG |
| | GGGTCTGTATAACGAGCTGCAGAAGGACAAGAT | KGHDGLYQGLS |
| | GGCCGAGGCCTACTCCGAGATCGGCATGAAAGG | TATKDTYDALH |
| | GGAGCGCCGCAGAGGAAAAGGTCACGATGGTCT | MQALPPR (SEQ |
| | GTACCAGGGGTTGAGCACCGCTACCAAGGATAC | ID NO: 278) |
| | TTACGACGCTCTGCACATGCAAGCTCTGCCACCC | |
| | CGG (SEQ ID NO: 242) | |
| 9-8 | ATGGCACTCCCCGTTACCGCCCTTCTGCTGCCTC | MALPVTALLLPL |
| | TCGCCTTGCTGCTGCACGCAGCCAGACCACAGG | ALLLHAARPQV |
| | TCAAGCTGGAGGAGTCTGGTGGCGGGCTCGTTC | KLEESGGGLVQA |
| | AAGCAGGTCGGAGTCTCCGCCTGTCTTGCGCAG | GRSLRLSCAASE |
| | CATCAGAGCATACCTTCTCCTCACACGTGATGGG | HTFSSHVMGWF |
| | GTGGTTCAGGCAAGCTCCCGGTAAGGAGAGGGA | RQAPGKERESVA |
| | GTCCGTGGCCGTTATCGGCTGGCGCGATATCAG | VIGWRDISTSYA |
| | CACCTCCTACGCAGACAGCGTTAAGGGCCGGTT | DSVKGRFTISRD |
| | CACTATCTCCAGGGACAACGCTAAGAAGACACT | NAKKTLYLQMN |
| | CTACCTCCAGATGAACAGTCTGAAGCCCGAGGA | SLKPEDTAVYYC |
| | CACCGCAGTGTACTATTGCGCTGCTCGGCGGATC | AARRIDAADFDS |
| | GATGCTGCCGACTTCGACAGCTGGGGTCAAGGG | WGQGTQVTVSS |
| | ACCCAGGTCACCGTTTCCAGCGGAGGTGGCGGA | GGGGSGGGGSG |
| | AGTGGTGGCGGAGGATCAGGTGGTGGAGGCTCC | GGGSEVQLVESG |
| | GAGGTCCAGCTGGTGGAATCAGGAGGCGGCTTG | GGLVQAGGSLR |
| | GTGCAGGCTGGTGGGTCTTTGCGGTTGTCCTGCG | LSCAASGRTFTM |
| | CAGCTTCCGGCAGGACCTTCACCATGGGATGGT | GWFRQAPGKER |
| | TCAGACAAGCCCCAGGTAAGGAGCGGGAGTTTG | EFVAAISLSPTLA |
| | TGGCCGCAATCTCACTGTCTCCCACCCTCGCTTA | YYAESVKGRFTI |
| | CTACGCCGAGAGTGTGAAGGGGCGCTTCACAAT | SRDNAKNTVVL |
| | CAGTCGCGACAACGCAAAGAACACCGTCGTCCT | QMNSLKPEDTAL |
| | GCAAATGAACTCCCTGAAGCCTGAGGATACCGC | YYCAADRKSVM |
| | ACTCTATTACTGCGCCGCCGATCGGAAGAGCGT | SIRPDYWGQGTQ |
| | CATGTCCATCCGGCCCGACTATTGGGGCCAAGG | VTVSSTSTTTPAP |
| | CACCCAAGTGACCGTCAGCTCCACCTCCACAAC | RPPTPAPTIASQP |
| | CACTCCCGCCCCAAGACCACCTACCCCAGCCCC | LSLRPEACRPAA |
| | AACAATCGCATCCCAGCCTCTGTCCCTTCGGCCC | GGAVHTRGLDF |
| | GAAGCTTGTCGCCCTGCAGCAGGTGGAGCAGTG | ACDIYIWAPLAG |
| | CACACCCGGGGACTGGACTTCGCCTGCGACATC | TCGVLLLSLVITL |
| | TACATCTGGGCACCCCTGGCTGGAACCTGCGGC | YCRSKRSRLLHS |
| | GTGTTGCTGCTGAGCCTGGTGATCACCCTCTACT | DYMNMTPRRPG |
| | GCCGCTCTAAGAGAAGCCGGCTGCTGCATAGCG | PTRKHYQPYAPP |
| | ACTACATGAACATGACCCCTAGGAGACCAGGAC | RDFAAYRSRVKF |
| | CCACCCGGAAGCACTACCAGCCTTACGCTCCTCC | SRSADAPAYQQ |
| | ACGGGATTTCGCTGCTTACCGCAGCCGGGTGAA | GQNQLYNELNL |
| | GTTTTCCAGGTCAGCTGACGCCCCTGCCTACCAG | GRREEYDVLDK |
| | CAGGGCCAGAACCAATTGTACAACGAACTGAAT | RRGRDPEMGGK |
| | CTGGGACGGCGCGAGGAATACGACGTCCTGGAC | PRRKNPQEGLYN |
| | AAGAGGCGGGGTAGAGATCCCGAGATGGGCGG | ELQKDKMAEAY |
| | GAAACCTCGGCGGAAGAACCCTCAGGAGGGGCT | SEIGMKGERRRG |
| | CTACAACGAGCTGCAGAAGGATAAGATGGCCGA | KGHDGLYQGLS |
| | AGCCTACTCCGAGATCGGGATGAAGGGTGAACG | TATKDTYDALH |
| | GAGGAGGGGCAAGGGACACGACGGCCTGTATC | MQALPPR (SEQ |
| | AGGGCCTCAGCACCGCTACCAAGGACACCTACG | ID NO: 283) |
| | ACGCCCTGCACATGCAGGCTCTCCCACCACGG | |
| | (SEQ ID NO: 247) | |
| 9-9 | ATGGCTCTTCCCGTCACCGCTTTGCTGCTGCCCC | MALPVTALLLPL |
| | TGGCACTCCTCCTCCATGCTGCTCGGCCTCAGGT | ALLLHAARPQV |
| | GAAGCTGGAGGAGAGTGGTGGCGGTCTGGTGCA | KLEESGGGLVQA |
| | AGCTGGCAGATCTCTGCGCCTGTCTTGCGCAGCC | GRSLRLSCAASE |
| | AGCGAACACACCTTCTCCTCCCACGTGATGGGG | HTFSSHVMGWF |
| | TGGTTTCGGCAGGCACCCGGGAAAGAGCGCGAG | RQAPGKERESVA |
| | TCCGTCGCAGTCATCGGGTGGCGGGACATCTCT | VIGWRDISTSYA |
| | ACCAGCTACGCAGATTCCGTCAAGGGCCGGTTC | DSVKGRFTISRD |
| | ACCATTTCCCGGGATAACGCTAAGAAGACCCTC | NAKKTLYLQMN |
| | TACCTGCAAATGAACTCTCTGAAGCCCGAAGAC | SLKPEDTAVYYC |
| | ACCGCCGTCTACTATTGCGCAGCAAGGCGCATC | AARRIDAADFDS |
| | GACGCTGCCGACTTCGACTCTTGGGGCCAAGGA | WGQGTQVTVSS |
| | ACCCAGGTCACCGTGTCTTCCGGAGGAGGAGGC | GGGGSGGGGSG |
| | TCCGGTGGTGGAGGTTCTGGAGGTGGCGGCTCA | GGGSEVQLVESG |
| | GAGGTGCAGCTCGTGGAGAGCGGTGGTGGACTC | GGLVQAGGSLR |
| | GTTCAGGCAGGCGGCAGTTTGCGGCTGTCCTGT | LSCAASGRTFTM |
| | GCAGCCTCCGGTCGCACTTTCACTATGGGATGGT | GWFRQAPGKER |
| | TCCGCCAGGCTCCTGGTAAAGAAAGGGAGTTCG | EFVAAISLSPTLA |
| | TGGCCGCCATCAGTCTGAGCCCCACCCTCGCATA | YYAESVKGRFTI |
| | CTACGCCGAGAGCGTGAAGGGTAGGTTCACTAT | SRDNAKNTVVL |
| | CAGCCGGGACAACGCCAAGAACACCGTGGTGCT | QMNSLKPEDTAL |
| | CCAGATGAATTCCCTGAAGCCTGAGGATACCGC | YYCAADRKSVM |
| | CCTCTACTACTGCGCTGCCGACCGCAAGAGCGT | SIRPDYWGQGTQ |
| | GATGAGCATCCGGCCTGACTATTGGGGTCAGGG | VTVSSIEVMYPP |
| | GACACAGGTGACCGTCAGCAGCATCGAGGTGAT | PYLDNEKSNGTII |
| | GTATCCACCACCCTACCTCGACAACGAGAAGTC | HVKGKHLCPSPL |
| | CAACGGCACCATCATCCACGTCAAGGGGAAGCA | FPGPSKPFWVLV |
| | CCTCTGCCCTTCCCCTCTGTTCCCTGGCCCCTCA | VVGGVLACYSL |
| | AAGCCCTTCTGGGTCCTGGTGGTGGTTGGTGGG | LVTVAFIIFWVRS |
| | GTGCTGGCTTGCTACTCCCTGCTCGTGACCGTGG | KRSRLLHSDYM |
| | CTTTCATCATCTTCTGGGTTCGGAGCAAACGGTC | NMTPRRPGPTRK |
| | CAGACTGCTGCACTCCGACTACATGAACATGAC | HYQPYAPPRDFA |
| | CCCAAGAAGACCTGGGCCCACACGGAAGCATTA | AYRSRVKFSRSA |
| | CCAACCCTATGCACCACCTCGGGATTTCGCCGCC | DAPAYQQGQNQ |
| | TACAGATCCCGGGTCAAGTTCTCCAGGTCCGCC | LYNELNLGRREE |
| | GATGCACCAGCCTATCAGCAGGGGCAAAACCAG | YDVLDKRRGRD |
| | CTGTATAATGAGCTGAACCTTGGACGGCGCGAG | PEMGGKPRRKN |
| | GAGTACGACGTGCTCGACAAAAGACGCGGTCGC | PQEGLYNELQKD |
| | GACCCAGAGATGGGCGGCAAGCCTAGACGCAA | KMAEAYSEIGM |
| | GAATCCCCAGGAGGGGCTCTATAACGAGTTGCA | KGERRRGKGHD |
| | GAAGGATAAGATGGCCGAGGCCTACAGCGAGAT | GLYQGLSTATKD |
| | CGGGATGAAAGGCGAAAGACGGCGCGGAAAGG | TYDALHMQALP |
| | GTCACGACGGACTCTACCAGGGCCTGAGCACAG | PR (SEQ ID NO: |
| | CCACCAAAGACACCTACGACGCTCTGCATATGC | 284) |
| | AAGCACTGCCTCCCCGG (SEQ ID NO: 248) | |
| 9-10 | ATGGCTCTCCCCGTGACCGCTCTCCTCCTTCCCT | MALPVTALLLPL |
| | TGGCCTTGTTGCTGCACGCCGCAAGGCCTCAGGT | ALLLHAARPQV |
| | GAAGCTGGAGGAATCCGGAGGCGGACTCGTCCA | KLEESGGGLVQA |
| | AGCAGGTCGGTCCCTCAGGCTGTCTTGCGCTGCC | GRSLRLSCAASE |
| | AGCGAGCACACCTTCTCTAGCCACGTGATGGGT | HTFSSHVMGWF |
| | TGGTTCAGACAGGCCCCAGGAAAGGAGCGGGA | RQAPGKERESVA |
| | ATCCGTGGCAGTGATCGGCTGGCGGGACATCAG | VIGWRDISTSYA |
| | CACCTCCTACGCCGACTCCGTCAAGGGCCGGTTC | DSVKGRFTISRD |
| | ACCATCAGCCGCGACAACGCCAAGAAGACCCTG | NAKKTLYLQMN |
| | TACCTGCAGATGAACAGCCTCAAGCCTGAGGAC | SLKPEDTAVYYC |
| | ACCGCCGTGTACTACTGCGCCGCCAGAAGGATC | AARRIDAADFDS |
| | GACGCCGCAGACTTCGACTCCTGGGGTCAGGGA | WGQGTQVTVSS |
| | ACCCAGGTGACCGTGTCCTCCACCTCTACCACCA | TSTTTPAPRPPTP |
| | CACCAGCACCCAGACCTCCTACTCCCGCTCCCAC | APTIASQPLSLRP |
| | CATCGCTTCCCAGCCCCTGTCCCTCAGACCCGAA | EACRPAAGGAV |
| | GCCTGCAGACCAGCAGCTGGCGGTGCAGTGCAC | HTRGLDFACDIY |
| | ACCAGGGGTCTTGACTTCGCCTGTGACATCTACA | IWAPLAGTCGVL |
| | TCTGGGCTCCACTGGCTGGGACTTGCGGCGTTCT | LLSLVITLYCKR |
| | GCTGCTGAGCCTGGTGATCACCCTGTACTGCAA | GRKKLLYIFKQP |
| | GCGGGGCCGGAAGAAGCTGCTCTACATCTTCAA | FMRPVQTTQEED |
| | GCAGCCTTTCATGCGGCCCGTTCAGACCACCCA | GCSCRFPEEEEG |
| | GGAGGAAGACGGGTGCAGTTGCCGCTTCCCTGA | GCELRVKFSRSA |
| | GGAGGAGGAGGGAGGATGCGAGCTGCGGGTCA | DAPAYQQGQNQ |
| | AGTTCTCTCGGTCCGCTGATGCCCCAGCCTACCA | LYNELNLGRREE |
| | GCAGGGCCAGAACCAGCTCTATAACGAGCTGAA | YDVLDKRRGRD |
| | CCTCGGTAGGCGGGAGGAGTACGACGTCCTGGA | PEMGGKPRRKN |
| | CAAAAGGAGGGGACGGGATCCTGAGATGGGAG | PQEGLYNELQKD |
| | GAAAGCCACGGCGGAAGAACCCTCAAGAGGGG | KMAEAYSEIGM |
| | CTGTACAACGAACTCCAGAAGGACAAGATGGCT | KGERRRGKGHD |
| | GAGGCTTATTCTGAGATCGGCATGAAGGGAGAG | GLYQGLSTATKD |
| | CGCAGACGCGGCAAGGGACACGATGGCCTGTAT | TYDALHMQALP |
| | CAGGGACTGAGCACCGCCACCAAGGATACCTAC | PR (SEQ ID NO: |
| | GACGCCCTCCATATGCAGGCTCTCCCACCACGG | 328) |
| | (SEQ ID NO: 312) | |
| 9-11 | ATGGCACTGCCCGTCACAGCTCTGCTGCTGCCCT | MALPVTALLLPL |
| | TGGCCCTCTTGTTGCACGCAGCACGGCCAGAGG | ALLLHAARPEVQ |
| | TGCAACTTGTCGAGAGCGGTGGTGGCCTTGTGC | LVESGGGLVQA |
| | AGGCCGGAGGCAGTTTGCGCCTCAGTTGTGCAG | GGSLRLSCAASG |
| | CTTCTGGCCGGACCTTCACCATGGGCTGGTTCAG | RTFTMGWFRQA |
| | ACAGGCCCCTGGTAAGGAACGCGAGTTCGTGGC | PGKEREFVAAIS |
| | CGCCATCAGCCTGTCCCCAACCCTGGCCTACTAC | LSPTLAYYAESV |
| | GCAGAGAGCGTGAAGGGTCGGTTCACCATCTCC | KGRFTISRDNAK |
| | CGCGACAACGCAAAGAACACCGTGGTGCTCCAG | NTVVLQMNSLK |
| | ATGAACTCCCTGAAGCCAGAAGACACCGCCCTG | PEDTALYYCAA |
| | TACTACTGCGCAGCCGACCGGAAGAGCGTGATG | DRKSVMSIRPDY |
| | TCCATCCGCCCTGACTACTGGGGCCAAGGCACA | WGQGTQVTVSS |
| | CAGGTCACAGTGTCCAGCACCTCCACTACCACTC | TSTTTPAPRPPTP |
| | CAGCTCCACGCCCTCCAACACCCGCACCAACCA | APTIASQPLSLRP |
| | TCGCCAGCCAGCCTCTGAGTCTGAGACCCGAAG | EACRPAAGGAV |
| | CATGCCGGCCAGCTGCTGGAGGTGCCGTGCACA | HTRGLDFACDIY |
| | CCAGAGGGCTGGACTTCGCCTGCGACATCTACA | IWAPLAGTCGVL |
| | TCTGGGCTCCTCTGGCCGGAACTTGCGGGGTGCT | LLSLVITLYCKR |
| | GCTCCTCTCACTGGTCATCACCCTGTACTGCAAG | GRKKLLYIFKQP |
| | AGGGGCAGGAAGAAGCTCCTGTACATCTTCAAG | FMRPVQTTQEED |
| | CAGCCCTTCATGCGGCCAGTCCAGACAACCCAG | GCSCRFPEEEEG |
| | GAGGAAGACGGATGCAGCTGTCGCTTCCCCGAG | GCELRVKFSRSA |
| | GAGGAGGAAGGCGGCTGCGAATTGCGGGTCAA | DAPAYQQGQNQ |
| | GTTCAGCAGATCCGCTGACGCTCCTGCCTACCAA | LYNELNLGRREE |
| | CAGGGACAGAACCAGCTCTACAACGAGCTGAAC | YDVLDKRRGRD |
| | CTGGGAAGGCGGGAGGAGTACGACGTCCTGGAC | PEMGGKPRRKN |
| | AAGAGAAGAGGACGCGACCCCGAGATGGGAGG | PQEGLYNELQKD |
| | TAAGCCCAGACGCAAGAACCCTCAAGAGGGACT | KMAEAYSEIGM |
| | GTATAACGAGCTGCAGAAGGACAAGATGGCCGA | KGERRRGKGHD |
| | GGCCTACAGCGAGATCGGCATGAAGGGTGAAAG | GLYQGLSTATKD |
| | AAGACGGGGAAAGGGGCACGACGGTCTGTATCA | TYDALHMQALP |
| | GGGGCTCTCCACCGCAACCAAGGATACCTATGA | PR (SEQ ID NO: |
| | CGCTCTGCACATGCAGGCACTCCCTCCACGC | 329) |
| | (SEQ ID NO: 313) | |
| 9-12 | ATGGCACTGCCCGTGACCGCTCTGCTGCTGCCTC | MALPVTALLLPL |
| | TCGCCCTGCTGCTGCATGCTGCCAGGCCCCAAGT | ALLLHAARPQV |
| | CAAGCTCGAGGAGTCTGGTGGTGGGCTCGTCCA | KLEESGGGLVQA |
| | AGCTGGCAGGTCCCTGAGACTGAGTTGTGCCGC | GRSLRLSCAASE |
| | CTCCGAGCACACTTTCAGCTCTCACGTGATGGGC | HTFSSHVMGWF |
| | TGGTTCAGGCAGGCACCCGGTAAGGAGCGGGAG | RQAPGKERESVA |
| | TCTGTGGCTGTTATCGGGTGGCGGGATATCTCTA | VIGWRDISTSYA |
| | CCTCCTACGCCGACTCCGTCAAGGGCCGGTTCAC | DSVKGRFTISRD |
| | CATCTCCAGGGACAACGCAAAGAAGACCCTCTA | NAKKTLYLQMN |
| | CCTCCAGATGAACTCACTCAAGCCCGAGGACAC | SLKPEDTAVYYC |
| | CGCTGTGTACTACTGCGCAGCCAGACGCATCGA | AARRIDAADFDS |
| | TGCCGCAGACTTCGACTCCTGGGGCCAAGGTAC | WGQGTQVTVSSI |
| | CCAAGTGACAGTGTCCAGCATCGAGGTGATGTA | EVMYPPPYLDNE |
| | CCCACCTCCCTACCTCGACAACGAGAAGAGCAA | KSNGTIIHVKGK |
| | CGGCACCATCATCCACGTGAAGGGGAAGCACCT | HLCPSPLFPGPSK |
| | GTGTCCCTCTCCCCTTTTCCCAGGACCCTCCAAG | PFWVLVVVGGV |
| | CCATTCTGGGTCCTGGTCGTTGTCGGAGGCGTGC | LACYSLLVTVAF |
| | TCGCTTGCTATTCCCTGCTCGTCACCGTGGCCTT | IIFWVRSKRSRLL |
| | CATCATCTTCTGGGTGCGGTCCAAGAGATCCCG | HSDYMNMTPRR |
| | GCTGCTGCACTCTGATTACATGAACATGACACCC | PGPTRKHYQPYA |
| | AGGAGGCCAGGGCCTACCAGGAAGCACTACCAG | PPRDFAAYRSRV |
| | CCCTACGCTCCTCCACGCGACTTCGCAGCATACC | KFSRSADAPAYQ |
| | GGTCCCGCGTCAAGTTCTCCCGGTCCGCAGATGC | QGQNQLYNELN |
| | TCCAGCCTATCAGCAGGGCCAGAACCAGCTGTA | LGRREEYDVLD |
| | CAACGAACTCAATTTGGGGAGGCGCGAGGAGTA | KRRGRDPEMGG |
| | TGATGTGCTCGATAAGAGAAGGGGCCGGGATCC | KPRRKNPQEGLY |
| | TGAGATGGGAGGCAAGCCCAGACGGAAGAACC | NELQKDKMAEA |
| | CTCAGGAGGGCCTGTACAATGAGCTGCAGAAGG | YSEIGMKGERRR |
| | ACAAGATGGCCGAGGCCTACTCCGAGATCGGGA | GKGHDGLYQGL |
| | TGAAGGGTGAAAGAAGGAGGGGTAAGGGGCAC | STATKDTYDALH |
| | GACGGGCTCTACCAAGGCCTGAGCACCGCTACC | MQALPPR(SEQ |
| | AAGGACACCTACGATGCACTGCATATGCAAGCC | ID NO: 330) |
| | CTGCCACCACGG (SEQ ID NO: 314) | |
| 9-13 | ATGGCCCTCCCTGTCACAGCCCTCTTGCTGCCCC | MALPVTALLLPL |
| | TCGCACTCCTTCTGCACGCAGCTCGGCCTGAGGT | ALLLHAARPEVQ |
| | CCAGCTGGTCGAGAGTGGAGGCGGACTGGTCCA | LVESGGGLVQA |
| | GGCAGGTGGGAGTCTTCGGCTTTCCTGCGCAGCT | GGSLRLSCAASG |
| | TCCGGACGGACCTTCACCATGGGTTGGTTCCGGC | RTFTMGWFRQA |
| | AGGCACCTGGGAAGGAGAGGGAGTTCGTTGCCG | PGKEREFVAAIS |
| | CTATCAGCCTCTCACCAACCCTGGCCTACTATGC | LSPTLAYYAESV |
| | AGAGAGCGTCAAGGGCCGCTTCACCATCAGCCG | KGRFTISRDNAK |
| | CGACAACGCCAAGAACACCGTCGTGCTGCAGAT | NTVVLQMNSLK |
| | GAACTCCCTCAAGCCCGAGGATACCGCCCTGTA | PEDTALYYCAA |
| | CTACTGCGCTGCCGATCGGAAGTCCGTCATGTCC | DRKSVMSIRPDY |
| | ATTCGGCCCGACTACTGGGGACAGGGCACACAG | WGQGTQVTVSSI |
| | GTGACCGTCAGCAGCATCGAGGTCATGTACCCT | EVMYPPPYLDNE |
| | CCACCCTACCTGGACAACGAGAAGAGCAACGGG | KSNGTIIHVKGK |
| | ACCATCATCCACGTGAAGGGGAAGCACCTCTGT | HLCPSPLFPGPSK |
| | CCAAGTCCCCTCTTCCCAGGACCCTCCAAGCCAT | PFWVLVVVGGV |
| | TCTGGGTCCTCGTGGTGGTTGGAGGAGTGCTCGC | LACYSLLVTVAF |
| | CTGCTACTCTCTGCTGGTGACCGTCGCCTTCATC | IIFWVRSKRSRLL |
| | ATCTTCTGGGTGCGGTCCAAGCGGTCTCGCCTCC | HSDYMNMTPRR |
| | TCCACTCCGACTACATGAACATGACACCACGCA | PGPTRKHYQPYA |
| | GACCTGGGCCCACTAGGAAGCACTATCAGCCCT | PPRDFAAYRSRV |
| | ATGCACCACCCCGGGATTTCGCAGCCTACCGGT | KFSRSADAPAYQ |
| | CACGGGTGAAGTTCAGCAGATCCGCAGACGCAC | QGQNQLYNELN |
| | CAGCCTACCAGCAGGGGCAGAACCAGCTGTATA | LGRREEYDVLD |
| | ACGAGCTGAACCTCGGTCGCAGGGAGGAGTACG | KRRGRDPEMGG |
| | ATGTCCTGGATAAGAGAAGGGGCAGGGATCCCG | KPRRKNPQEGLY |
| | AGATGGGTGGCAAGCCCAGACGGAAGAATCCTC | NELQKDKMAEA |
| | AGGAGGGGCTCTACAACGAGCTGCAGAAGGAC | YSEIGMKGERRR |
| | AAGATGGCCGAGGCTTACTCAGAGATCGGCATG | GKGHDGLYQGL |
| | AAAGGGGAGAGGAGGCGCGGAAAAGGCCACGA | STATKDTYDALH |
| | CGGCCTCTACCAGGGACTGTCCACCGCAACCAA | MQALPPR (SEQ |
| | GGATACCTACGACGCCCTGCACATGCAAGCCCT | ID NO: 331) |
| | CCCACCTCGG (SEQ ID NO: 315) | |
| 9-14 | ATGGCTCTCCCCGTGACCGCTCTCCTCCTTCCCT | MALPVTALLLPL |
| | TGGCCTTGTTGCTGCACGCCGCAAGGCCTCAGGT | ALLLHAARPQV |
| | GAAGCTGGAGGAATCCGGAGGCGGACTCGTCCA | KLEESGGGLVQA |
| | AGCAGGTCGGTCCCTCAGGCTGTCTTGCGCTGCC | GRSLRLSCAASE |
| | AGCGAGCACACCTTCTCTAGCCACGTGATGGGT | HTFSSHVMGWF |
| | TGGTTCAGACAGGCCCCAGGAAAGGAGCGGGA | RQAPGKERESVA |
| | ATCCGTGGCAGTGATCGGCTGGCGGGACATCAG | VIGWRDISTSYA |
| | CACCTCCTACGCCGACTCCGTCAAGGGCCGGTTC | DSVKGRFTISRD |
| | ACCATCAGCCGCGACAACGCCAAGAAGACCCTG | NAKKTLYLQMN |
| | TACCTGCAGATGAACAGCCTCAAGCCTGAGGAC | SLKPEDTAVYYC |
| | ACCGCCGTGTACTACTGCGCCGCCAGAAGGATC | AARRIDAADFDS |
| | GACGCCGCAGACTTCGACTCCTGGGGTCAGGGA | WGQGTQVTVSS |
| | ACCCAGGTGACCGTGTCCTCCACCTCTACCACCA | TSTTTPAPRPPTP |
| | CACCAGCACCCAGACCTCCTACTCCCGCTCCCAC | APTIASQPLSLRP |
| | CATCGCTTCCCAGCCCCTGTCCCTCAGACCCGAA | EACRPAAGGAV |
| | GCCTGCAGACCAGCAGCTGGCGGTGCAGTGCAC | HTRGLDFACDIY |
| | ACCAGGGGTCTTGACTTCGCCTGTGACATCTACA | IWAPLAGTCGVL |
| | TCTGGGCTCCACTGGCTGGGACTTGCGGCGTTCT | LLSLVITLYCKR |
| | GCTGCTGAGCCTGGTGATCACCCTGTACTGCAA | GRKKLLYIFKQP |
| | GCGGGGCCGGAAGAAGCTGCTCTACATCTTCAA | FMRPVQTTQEED |
| | GCAGCCTTTCATGCGGCCCGTTCAGACCACCCA | GCSCRFPEEEEG |
| | GGAGGAAGACGGGTGCAGTTGCCGCTTCCCTGA | GCELRVKFSRSA |
| | GGAGGAGGAGGGAGGATGCGAGCTGCGGGTCA | DAPAYQQGQNQ |
| | AGTTCTCTCGGTCCGCTGATGCCCCAGCCTACCA | LYNELNLGRREE |
| | GCAGGGCCAGAACCAGCTCTATAACGAGCTGAA | YDVLDKRRGRD |
| | CCTCGGTAGGCGGGAGGAGTACGACGTCCTGGA | PEMGGKPRRKN |
| | CAAAAGGAGGGGACGGGATCCTGAGATGGGAG | PQEGLYNELQKD |
| | GAAAGCCACGGCGGAAGAACCCTCAAGAGGGG | KMAEAYSEIGM |
| | CTGTACAACGAACTCCAGAAGGACAAGATGGCT | KGERRRGKGHD |
| | GAGGCTTATTCTGAGATCGGCATGAAGGGAGAG | GLYQGLSTATKD |
| | CGCAGACGCGGCAAGGGACACGATGGCCTGTAT | TYDALHMQALP |
| | CAGGGACTGAGCACCGCCACCAAGGATACCTAC | PRGSGEGRGSLL |
| | GACGCCCTCCATATGCAGGCTCTCCCACCACGG | TCGDVEENPGP |
| | GGATCTGGCGAAGGCAGAGGATCTCTGCTGACA | MALPVTALLLPL |
| | TGCGGCGACGTGGAAGAGAACCCTGGACCTATG | ALLLHAARPEVQ |
| | GCCCTCCCTGTCACAGCCCTCTTGCTGCCCCTCG | LVESGGGLVQA |
| | CACTCCTTCTGCACGCAGCTCGGCCTGAGGTCCA | GGSLRLSCAASG |
| | GCTGGTCGAGAGTGGAGGCGGACTGGTCCAGGC | RTFTMGWFRQA |
| | AGGTGGGAGTCTTCGGCTTTCCTGCGCAGCTTCC | PGKEREFVAAIS |
| | GGACGGACCTTCACCATGGGTTGGTTCCGGCAG | LSPTLAYYAESV |
| | GCACCTGGGAAGGAGAGGGAGTTCGTTGCCGCT | KGRFTISRDNAK |
| | ATCAGCCTCTCACCAACCCTGGCCTACTATGCAG | NTVVLQMNSLK |
| | AGAGCGTCAAGGGCCGCTTCACCATCAGCCGCG | PEDTALYYCAA |
| | ACAACGCCAAGAACACCGTCGTGCTGCAGATGA | DRKSVMSIRPDY |
| | ACTCCCTCAAGCCCGAGGATACCGCCCTGTACT | WGQGTQVTVSSI |
| | ACTGCGCTGCCGATCGGAAGTCCGTCATGTCCAT | EVMYPPPYLDNE |
| | TCGGCCCGACTACTGGGGACAGGGCACACAGGT | KSNGTIIHVKGK |
| | GACCGTCAGCAGCATCGAGGTCATGTACCCTCC | HLCPSPLFPGPSK |
| | ACCCTACCTGGACAACGAGAAGAGCAACGGGAC | PFWVLVVVGGV |
| | CATCATCCACGTGAAGGGGAAGCACCTCTGTCC | LACYSLLVTVAF |
| | AAGTCCCCTCTTCCCAGGACCCTCCAAGCCATTC | IIFWVRSKRSRLL |
| | TGGGTCCTCGTGGTGGTTGGAGGAGTGCTCGCCT | HSDYMNMTPRR |
| | GCTACTCTCTGCTGGTGACCGTCGCCTTCATCAT | PGPTRKHYQPYA |
| | CTTCTGGGTGCGGTCCAAGCGGTCTCGCCTCCTC | PPRDFAAYRSRV |
| | CACTCCGACTACATGAACATGACACCACGCAGA | KFSRSADAPAYQ |
| | CCTGGGCCCACTAGGAAGCACTATCAGCCCTAT | QGQNQLYNELN |
| | GCACCACCCCGGGATTTCGCAGCCTACCGGTCA | LGRREEYDVLD |
| | CGGGTGAAGTTCAGCAGATCCGCAGACGCACCA | KRRGRDPEMGG |
| | GCCTACCAGCAGGGGCAGAACCAGCTGTATAAC | KPRRKNPQEGLY |
| | GAGCTGAACCTCGGTCGCAGGGAGGAGTACGAT | NELQKDKMAEA |
| | GTCCTGGATAAGAGAAGGGGCAGGGATCCCGAG | YSEIGMKGERRR |
| | ATGGGTGGCAAGCCCAGACGGAAGAATCCTCAG | GKGHDGLYQGL |
| | GAGGGGCTCTACAACGAGCTGCAGAAGGACAA | STATKDTYDALH |
| | GATGGCCGAGGCTTACTCAGAGATCGGCATGAA | MQALPPR (SEQ |
| | AGGGGAGAGGAGGCGCGGAAAAGGCCACGACG | ID NO: 332) |
| | GCCTCTACCAGGGACTGTCCACCGCAACCAAGG | |
| | ATACCTACGACGCCCTGCACATGCAAGCCCTCC | |
| | CACCTCGG (SEQ ID NO: 316) | |
| 9-15 | ATGGCACTGCCCGTCACAGCTCTGCTGCTGCCCT | MALPVTALLLPL |
| | TGGCCCTCTTGTTGCACGCAGCACGGCCAGAGG | ALLLHAARPEVQ |
| | TGCAACTTGTCGAGAGCGGTGGTGGCCTTGTGC | LVESGGGLVQA |
| | AGGCCGGAGGCAGTTTGCGCCTCAGTTGTGCAG | GGSLRLSCAASG |
| | CTTCTGGCCGGACCTTCACCATGGGCTGGTTCAG | RTFTMGWFRQA |
| | ACAGGCCCCTGGTAAGGAACGCGAGTTCGTGGC | PGKEREFVAAIS |
| | CGCCATCAGCCTGTCCCCAACCCTGGCCTACTAC | LSPTLAYYAESV |
| | GCAGAGAGCGTGAAGGGTCGGTTCACCATCTCC | KGRFTISRDNAK |
| | CGCGACAACGCAAAGAACACCGTGGTGCTCCAG | NTVVLQMNSLK |
| | ATGAACTCCCTGAAGCCAGAAGACACCGCCCTG | PEDTALYYCAA |
| | TACTACTGCGCAGCCGACCGGAAGAGCGTGATG | DRKSVMSIRPDY |
| | TCCATCCGCCCTGACTACTGGGGCCAAGGCACA | WGQGTQVTVSS |
| | CAGGTCACAGTGTCCAGCACCTCCACTACCACTC | TSTTTPAPRPPTP |
| | CAGCTCCACGCCCTCCAACACCCGCACCAACCA | APTIASQPLSLRP |
| | TCGCCAGCCAGCCTCTGAGTCTGAGACCCGAAG | EACRPAAGGAV |
| | CATGCCGGCCAGCTGCTGGAGGTGCCGTGCACA | HTRGLDFACDIY |
| | CCAGAGGGCTGGACTTCGCCTGCGACATCTACA | IWAPLAGTCGVL |
| | TCTGGGCTCCTCTGGCCGGAACTTGCGGGGTGCT | LLSLVITLYCKR |
| | GCTCCTCTCACTGGTCATCACCCTGTACTGCAAG | GRKKLLYIFKQP |
| | AGGGGCAGGAAGAAGCTCCTGTACATCTTCAAG | FMRPVQTTQEED |
| | CAGCCCTTCATGCGGCCAGTCCAGACAACCCAG | GCSCRFPEEEEG |
| | GAGGAAGACGGATGCAGCTGTCGCTTCCCCGAG | GCELRVKFSRSA |
| | GAGGAGGAAGGCGGCTGCGAATTGCGGGTCAA | DAPAYQQGQNQ |
| | GTTCAGCAGATCCGCTGACGCTCCTGCCTACCAA | LYNELNLGRREE |
| | CAGGGACAGAACCAGCTCTACAACGAGCTGAAC | YDVLDKRRGRD |
| | CTGGGAAGGCGGGAGGAGTACGACGTCCTGGAC | PEMGGKPRRKN |
| | AAGAGAAGAGGACGCGACCCCGAGATGGGAGG | PQEGLYNELQKD |
| | TAAGCCCAGACGCAAGAACCCTCAAGAGGGACT | KMAEAYSEIGM |
| | GTATAACGAGCTGCAGAAGGACAAGATGGCCGA | KGERRRGKGHD |
| | GGCCTACAGCGAGATCGGCATGAAGGGTGAAAG | GLYQGLSTATKD |
| | AAGACGGGGAAAGGGGCACGACGGTCTGTATCA | TYDALHMQALP |
| | GGGGCTCTCCACCGCAACCAAGGATACCTATGA | PRGSGEGRGSLL |
| | CGCTCTGCACATGCAGGCACTCCCTCCACGCGG | TCGDVEENPGP |
| | ATCTGGCGAAGGCAGAGGATCTCTGCTGACATG | MALPVTALLLPL |
| | CGGCGACGTGGAAGAGAACCCTGGACCTATGGC | ALLLHAARPQV |
| | ACTGCCCGTGACCGCTCTGCTGCTGCCTCTCGCC | KLEESGGGLVQA |
| | CTGCTGCTGCATGCTGCCAGGCCCCAAGTCAAG | GRSLRLSCAASE |
| | CTCGAGGAGTCTGGTGGTGGGCTCGTCCAAGCT | HTFSSHVMGWF |
| | GGCAGGTCCCTGAGACTGAGTTGTGCCGCCTCC | RQAPGKERESVA |
| | GAGCACACTTTCAGCTCTCACGTGATGGGCTGGT | VIGWRDISTSYA |
| | TCAGGCAGGCACCCGGTAAGGAGCGGGAGTCTG | DSVKGRFTISRD |
| | TGGCTGTTATCGGGTGGCGGGATATCTCTACCTC | NAKKTLYLQMN |
| | CTACGCCGACTCCGTCAAGGGCCGGTTCACCAT | SLKPEDTAVYYC |
| | CTCCAGGGACAACGCAAAGAAGACCCTCTACCT | AARRIDAADFDS |
| | CCAGATGAACTCACTCAAGCCCGAGGACACCGC | WGQGTQVTVSSI |
| | TGTGTACTACTGCGCAGCCAGACGCATCGATGC | EVMYPPPYLDNE |
| | CGCAGACTTCGACTCCTGGGGCCAAGGTACCCA | KSNGTIIHVKGK |
| | AGTGACAGTGTCCAGCATCGAGGTGATGTACCC | HLCPSPLFPGPSK |
| | ACCTCCCTACCTCGACAACGAGAAGAGCAACGG | PFWVLVVVGGV |
| | CACCATCATCCACGTGAAGGGGAAGCACCTGTG | LACYSLLVTVAF |
| | TCCCTCTCCCCTTTTCCCAGGACCCTCCAAGCCA | IIFWVRSKRSRLL |
| | TTCTGGGTCCTGGTCGTTGTCGGAGGCGTGCTCG | HSDYMNMTPRR |
| | CTTGCTATTCCCTGCTCGTCACCGTGGCCTTCAT | PGPTRKHYQPYA |
| | CATCTTCTGGGTGCGGTCCAAGAGATCCCGGCT | PPRDFAAYRSRV |
| | GCTGCACTCTGATTACATGAACATGACACCCAG | KFSRSADAPAYQ |
| | GAGGCCAGGGCCTACCAGGAAGCACTACCAGCC | QGQNQLYNELN |
| | CTACGCTCCTCCACGCGACTTCGCAGCATACCGG | LGRREEYDVLD |
| | TCCCGCGTCAAGTTCTCCCGGTCCGCAGATGCTC | KRRGRDPEMGG |
| | CAGCCTATCAGCAGGGCCAGAACCAGCTGTACA | KPRRKNPQEGLY |
| | ACGAACTCAATTTGGGGAGGCGCGAGGAGTATG | NELQKDKMAEA |
| | ATGTGCTCGATAAGAGAAGGGGCCGGGATCCTG | YSEIGMKGERRR |
| | AGATGGGAGGCAAGCCCAGACGGAAGAACCCT | GKGHDGLYQGL |
| | CAGGAGGGCCTGTACAATGAGCTGCAGAAGGAC | STATKDTYDALH |
| | AAGATGGCCGAGGCCTACTCCGAGATCGGGATG | MQALPPR (SEQ |
| | AAGGGTGAAAGAAGGAGGGGTAAGGGGCACGA | ID NO: 333) |
| | CGGGCTCTACCAAGGCCTGAGCACCGCTACCAA | |
| | GGACACCTACGATGCACTGCATATGCAAGCCCT | |
| | GCCACCACGG (SEQ ID NO: 317) | |
| 9-16 | ATGGCACTCCCGGTAACCGCCTTATTGCTTCCCC | MALPVTALLLPL |
| | TTGCCCTCTTGCTCCACGCAGCACGCCCCGATAT | ALLLHAARPDIV |
| | AGTCTTGACTCAATCCCCACCCAGTTTGGCAATG | LTQSPPSLAMSL |
| | TCATTAGGCAAACGAGCAACAATTTCATGTAGG | GKRATISCRASE |
| | GCATCCGAAAGTGTAACGATTTTGGGGAGTCAT | SVTILGSHLIHW |
| | TTAATTCATTGGTACCAACAAAAGCCTGGACAA | YQQKPGQPPTLL |
| | CCCCCGACGCTCTTGATCCAATTAGCATCTAACG | IQLASNVQTGVP |
| | TCCAAACCGGAGTCCCCGCACGATTCTCAGGAT | ARFSGSGSRTDF |
| | CCGGTTCCCGGACTGATTTTACATTAACTATTGA | TLTIDPVEEDDV |
| | TCCGGTAGAGGAAGATGACGTCGCTGTCTATTA | AVYYCLQSRTIP |
| | TTGTCTTCAAAGTAGGACGATTCCACGGACATTC | RTFGGGTKLEIK |
| | GGTGGCGGAACTAAATTGGAGATTAAAGGTTCC | GSTSGSGKPGSG |
| | ACCTCTGGTAGTGGGAAACCCGGGTCCGGTGAA | EGSTKGQIQLVQ |
| | GGGTCCACTAAAGGCCAAATTCAACTCGTTCAA | SGPELKKPGETV |
| | TCCGGACCAGAACTGAAGAAGCCAGGAGAAACT | KISCKASGYTFT |
| | GTCAAAATAAGCTGTAAAGCTTCCGGTTATACA | DYSINWVKRAP |
| | TTTACAGATTATTCCATAAATTGGGTGAAAAGG | GKGLKWMGWIN |
| | GCGCCAGGAAAAGGGTTAAAGTGGATGGGTTGG | TETREPAYAYDF |
| | ATTAATACAGAGACTCGGGAACCTGCATATGCT | RGRFAFSLETSA |
| | TATGATTTTAGGGGAAGGTTTGCCTTTTCTCTGG | STAYLQINNLKY |
| | AGACTTCCGCTTCAACTGCTTATCTCCAAATTAA | EDTATYFCALDY |
| | TAATCTTAAATATGAGGACACAGCAACATACTT | SYAMDYWGQGT |
| | CTGTGCTTTGGACTATAGTTATGCTATGGATTAC | SVTVSSAAATTT |
| | TGGGGACAAGGAACCAGTGTCACTGTAAGTTCC | PAPRPPTPAPTIA |
| | GCTGCTGCGACGACCACTCCTGCACCGCGACCA | SQPLSLRPEACRP |
| | CCCACTCCTGCCCCTACTATTGCTAGTCAACCAC | AAGGAVHTRGL |
| | TTAGCTTGCGACCTGAGGCATGTCGGCCCGCGG | DFACDIYIWAPL |
| | CAGGTGGCGCAGTCCACACCAGGGGTTTAGACT | AGTCGVLLLSLV |
| | TTGCTTGTGATATTTATATTTGGGCACCACTCGC | ITLYCKRGRKKL |
| | CGGGACTTGCGGTGTTCTTCTCTTGTCCCTTGTT | LYIFKQPFMRPV |
| | ATAACTCTTTATTGTAAGCGCGGAAGGAAGAAA | QTTQEEDGCSCR |
| | TTGTTATATATTTTCAAACAACCTTTTATGCGAC | FPEEEEGGCELR |
| | CCGTACAAACAACTCAGGAAGAGGACGGGTGTT | VKFSRSADAPAY |
| | CTTGTCGGTTTCCAGAAGAGGAAGAGGGTGGGT | QQGQNQLYNEL |
| | GTGAACTCCGGGTCAAATTTAGTAGGTCAGCAG | NLGRREEYDVL |
| | ATGCGCCGGCGTACCAACAAGGCCAAAACCAAC | DKRRGRDPEMG |
| | TGTATAATGAACTCAATCTCGGTAGGCGTGAGG | GKPRRKNPQEGL |
| | AATATGATGTCCTTGATAAAAGGCGCGGGAGAG | YNELQKDKMAE |
| | ATCCAGAAATGGGCGGAAAACCACGGCGAAAG | AYSEIGMKGERR |
| | AATCCGCAGGAAGGGTTATATAACGAACTTCAA | RGKGHDGLYQG |
| | AAGGATAAAATGGCTGAAGCTTATTCCGAAATT | LSTATKDTYDAL |
| | GGCATGAAAGGAGAGCGACGTAGGGGCAAAGG | HMQALPPR (SEQ |
| | GCATGATGGCCTTTACCAAGGGCTCTCAACCGCT | ID NO: 279) |
| | ACAAAAGATACTTACGACGCTTTACATATGCAA | |
| | GCACTTCCACCCAGG (SEQ ID NO: 243) | |
| 9-17 | ATGGCTCTGCCTGTGACAGCTCTGCTGCTGCCTC | MALPVTALLLPL |
| | TGGCTCTGCTTCTGCATGCCGCCAGACCTGACAT | ALLLHAARPDIQ |
| | CCAGATGACTCAGAGCCCCAGCAGCCTGTCTGC | MTQSPSSLSASV |
| | CTCTGTGGGAGACAGAGTGACAATTACCTGCCG | GDRVTITCRASQ |
| | GGCCAGCCAGGATGTGAATACTGCTGTCGCCTG | DVNTAVAWYQQ |
| | GTATCAACAAAAGCCTGGCAAGGCCCCTAAGCT | KPGKAPKLLIYS |
| | CCTGATCTACAGCGCCAGCTTTCTGTACAGCGGC | ASFLYSGVPSRFS |
| | GTGCCCAGCAGATTCTCCGGAAGCAGAAGCGGC | GSRSGTDFTLTIS |
| | ACAGATTTCACACTGACCATAAGCAGCCTGCAG | SLQPEDFATYYC |
| | CCAGAGGATTTCGCCACCTACTATTGCCAGCAG | QQHYTTPPTFGQ |
| | CACTACACCACACCTCCAACCTTTGGCCAGGGC | GTKVEIKRTGST |
| | ACCAAGGTCGAGATTAAGAGAACAGGCAGCAC | SGSGKPGSGEGS |
| | ATCTGGCTCTGGCAAACCTGGATCTGGCGAGGG | EVQLVESGGGLV |
| | CTCTGAAGTCCAGCTGGTGGAATCTGGCGGAGG | QPGGSLRLSCAA |
| | ACTGGTTCAACCTGGCGGCTCTCTGAGACTGTCT | SGFNIKDTYIHW |
| | TGTGCCGCCTCCGGCTTCAACATCAAGGACACCT | VRQAPGKGLEW |
| | ACATCCACTGGGTCCGACAAGCCCCAGGCAAAG | VARIYPTNGYTR |
| | GACTTGAGTGGGTCGCCAGGATCTACCCCACCA | YADSVKGRFTIS |
| | ACGGCTACACCAGATACGCCGACTCTGTGAAGG | ADTSKNTAYLQ |
| | GCAGATTCACCATCTCTGCCGACACCAGCAAGA | MNSLRAEDTAV |
| | ATACCGCCTACCTGCAGATGAACTCCCTGAGAG | YYCSRWGGDGF |
| | CCGAAGATACCGCTGTGTATTACTGTTCCAGATG | YAMDVWGQGT |
| | GGGAGGCGACGGCTTCTACGCCATGGATGTTTG | LVTVSSIEVMYP |
| | GGGCCAAGGCACCCTCGTGACCGTTTCTTCTATC | PPYLDNEKSNGT |
| | GAAGTGATGTACCCTCCACCTTACCTGGACAAC | IIHVKGKHLCPSP |
| | GAGAAGTCCAACGGCACCATCATCCACGTGAAG | LFPGPSKPFWVL |
| | GGCAAGCACCTGTGTCCTTCTCCACTGTTCCCCG | VVVGGVLACYS |
| | GACCTAGCAAGCCTTTCTGGGTGCTCGTTGTTGT | LLVTVAFIIFWV |
| | TGGCGGCGTGCTGGCCTGTTACTCTCTGCTGGTT | RSKRSRLLHSDY |
| | ACCGTGGCCTTCATCATCTTTTGGGTCCGAAGCA | MNMTPRRPGPT |
| | AGCGGAGCCGGCTGCTGCACTCCGACTACATGA | RKHYQPYAPPRD |
| | ACATGACCCCTAGACGGCCCGGACCAACCAGAA | FAAYRSRVKFSR |
| | AGCACTACCAGCCTTACGCTCCTCCTAGAGACTT | SADAPAYQQGQ |
| | CGCCGCCTACCGGTCCAGAGTGAAGTTCAGCAG | NQLYNELNLGR |
| | ATCCGCCGATGCTCCCGCCTATCAGCAGGGCCA | REEYDVLDKRR |
| | AAACCAGCTGTACAACGAGCTGAACCTGGGGAG | GRDPEMGGKPR |
| | AAGAGAAGAGTACGACGTGCTGGACAAGCGGA | RKNPQEGLYNEL |
| | GAGGCAGAGATCCTGAAATGGGCGGCAAGCCCA | QKDKMAEAYSEI |
| | GACGGAAGAATCCTCAAGAGGGCCTGTATAATG | GMKGERRRGKG |
| | AGCTGCAGAAAGACAAGATGGCCGAGGCCTACA | HDGLYQGLSTAT |
| | GCGAGATCGGAATGAAGGGCGAGCGCAGAAGA | KDTYDALHMQA |
| | GGCAAGGGACACGATGGACTGTACCAGGGCCTG | LPPR (SEQ ID |
| | AGCACCGCCACCAAGGATACCTATGATGCCCTG | NO: 285) |
| | CACATGCAGGCCCTGCCTCCAAGA (SEQ ID NO: | |
| | 249) | |
| 9-18 | ATGGCTCTGCCTGTGACAGCTCTGCTGCTGCCTC | MALPVTALLLPL |
| | TGGCTCTGCTTCTGCATGCCGCCAGACCTGACAT | ALLLHAARPDIQ |
| | CCAGATGACTCAGAGCCCCAGCAGCCTGTCTGC | MTQSPSSLSASV |
| | CTCTGTGGGAGACAGAGTGACAATTACCTGCCG | GDRVTITCRASQ |
| | GGCCAGCCAGGATGTGAATACTGCTGTCGCCTG | DVNTAVAWYQQ |
| | GTATCAACAAAAGCCTGGCAAGGCCCCTAAGCT | KPGKAPKLLIYS |
| | CCTGATCTACAGCGCCAGCTTTCTGTACAGCGGC | ASFLYSGVPSRFS |
| | GTGCCCAGCAGATTCTCCGGAAGCAGAAGCGGC | GSRSGTDFTLTIS |
| | ACAGATTTCACACTGACCATAAGCAGCCTGCAG | SLQPEDFATYYC |
| | CCAGAGGATTTCGCCACCTACTATTGCCAGCAG | QQHYTTPPTFGQ |
| | CACTACACCACACCTCCAACCTTTGGCCAGGGC | GTKVEIKRTGST |
| | ACCAAGGTCGAGATTAAGAGAACAGGCAGCAC | SGSGKPGSGEGS |
| | ATCTGGCTCTGGCAAACCTGGATCTGGCGAGGG | EVQLVESGGGLV |
| | CTCTGAAGTCCAGCTGGTGGAATCTGGCGGAGG | QPGGSLRLSCAA |
| | ACTGGTTCAACCTGGCGGCTCTCTGAGACTGTCT | SGFNIKDTYIHW |
| | TGTGCCGCCTCCGGCTTCAACATCAAGGACACCT | VRQAPGKGLEW |
| | ACATCCACTGGGTCCGACAAGCCCCAGGCAAAG | VARIYPTNGYTR |
| | GACTTGAGTGGGTCGCCAGGATCTACCCCACCA | YADSVKGRFTIS |
| | ACGGCTACACCAGATACGCCGACTCTGTGAAGG | ADTSKNTAYLQ |
| | GCAGATTCACCATCTCTGCCGACACCAGCAAGA | MNSLRAEDTAV |
| | ATACCGCCTACCTGCAGATGAACTCCCTGAGAG | YYCSRWGGDGF |
| | CCGAAGATACCGCTGTGTATTACTGTTCCAGATG | YAMDVWGQGT |
| | GGGAGGCGACGGCTTCTACGCCATGGATGTTTG | LVTVSSTTTPAP |
| | GGGCCAAGGCACCCTCGTGACCGTTTCTTCTACC | RPPTPAPTIASQP |
| | ACCACACCAGCTCCTCGGCCTCCAACTCCTGCTC | LSLRPEACRPAA |
| | CTACAATTGCCAGCCAGCCTCTGTCTCTGAGGCC | GGAVHTRGLDF |
| | CGAAGCTTGTAGACCTGCTGCTGGCGGAGCCGT | ACDIYIWAPLAG |
| | GCATACAAGAGGACTGGATTTCGCCTGCGACAT | TCGVLLLSLVITL |
| | CTACATCTGGGCTCCTCTGGCCGGAACATGTGGC | YCKRGRKKLLYI |
| | GTTCTGCTGCTGAGCCTGGTCATCACCCTGTACT | FKQPFMRPVQTT |
| | GTAAGCGGGGCAGAAAGAAGCTGCTGTACATCT | QEEDGCSCRFPE |
| | TCAAGCAGCCCTTCATGCGGCCCGTGCAGACCA | EEEGGCELRVKF |
| | CACAAGAGGAAGATGGCTGCTCCTGCAGATTCC | SRSADAPAYQQ |
| | CCGAGGAAGAAGAAGGCGGCTGCGAGCTGAGA | GQNQLYNELNL |
| | GTGAAGTTCAGCAGATCCGCCGATGCTCCCGCC | GRREEYDVLDK |
| | TATCAGCAGGGCCAAAACCAGCTGTACAACGAG | RRGRDPEMGGK |
| | CTGAACCTGGGGAGAAGAGAAGAGTACGACGT | PRRKNPQEGLYN |
| | GCTGGACAAGCGGAGAGGCAGAGATCCTGAAAT | ELQKDKMAEAY |
| | GGGCGGCAAGCCCAGACGGAAGAATCCTCAAG | SEIGMKGERRRG |
| | AGGGCCTGTATAATGAGCTGCAGAAAGACAAGA | KGHDGLYQGLS |
| | TGGCCGAGGCCTACAGCGAGATCGGAATGAAGG | TATKDTYDALH |
| | GCGAGCGCAGAAGAGGCAAGGGACACGATGGA | MQALPPR (SEQ |
| | CTGTACCAGGGCCTGAGCACCGCCACCAAGGAT | ID NO: 286) |
| | ACCTATGATGCCCTGCACATGCAGGCCCTGCCTC | |
| | CAAGA (SEQ ID NO: 250) | |
| 9-19 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGAGCTGTTCTGCC | GSLRLSCSASGF |
| | AGCGGCTTCACCCTGGACTACTACATGATCGGCT | TLDYYMIGWFR |
| | GGTTCAGACAGGCCCCTGGCAAAGAGAGAGAG | QAPGKEREGLSS |
| | GGCCTGAGCAGCATCTCTCCTGCCGATGGCAGC | ISPADGSTYYAD |
| | ACCTACTACGCCGATTCTGTGAAGGGCAGATTC | SVKGRFTISRDSS |
| | ACCATCAGCCGGGACAGCAGCAAGAACACCGTG | KNTVYLQMNSL |
| | TACCTGCAGATGAACAGCCTGAGAGCCGAGGAC | RAEDTAVYYCA |
| | ACCGCCGTGTACTATTGTGCCGCCGGAAATGAG | AGNEATISWGFG |
| | GCCACAATCAGCTGGGGATTTGGCCCCTGGGAG | PWEYDYWGQGT |
| | TACGATTATTGGGGCCAGGGCACCATGGTCACC | MVTVSSIEVMYP |
| | GTGTCCTCTATCGAAGTGATGTACCCTCCTCCTT | PPYLDNEKSNGT |
| | ACCTGGACAACGAGAAGTCCAACGGCACCATCA | IIHVKGKHLCPSP |
| | TCCACGTGAAGGGAAAGCACCTGTGTCCTTCTCC | LFPGPSKPFWVL |
| | ACTGTTCCCCGGACCTAGCAAGCCTTTCTGGGTG | VVVGGVLACYS |
| | CTCGTTGTTGTTGGCGGCGTGCTGGCCTGTTACT | LLVTVAFIIFWV |
| | CTCTGCTGGTTACCGTGGCCTTCATCATCTTTTG | RSKRSRLLHSDY |
| | GGTCCGAAGCAAGCGGAGCCGGCTGCTGCACAG | MNMTPRRPGPT |
| | CGACTACATGAACATGACCCCTAGACGGCCCGG | RKHYQPYAPPRD |
| | ACCAACCAGAAAGCACTACCAGCCTTACGCTCC | FAAYRSRVKFSR |
| | TCCTAGAGACTTCGCCGCCTACCGGTCCAGAGT | SADAPAYQQGQ |
| | GAAGTTCAGCAGATCCGCTGATGCCCCTGCCTAT | NQLYNELNLGR |
| | CAGCAGGGCCAGAACCAGCTGTACAACGAGCTG | REEYDVLDKRR |
| | AACCTGGGGAGAAGAGAAGAGTACGACGTGCT | GRDPEMGGKPR |
| | GGACAAGCGGAGAGGCAGAGATCCTGAGATGG | RKNPQEGLYNEL |
| | GCGGCAAGCCCAGACGGAAGAATCCTCAAGAG | QKDKMAEAYSEI |
| | GGACTGTATAATGAGCTGCAGAAAGACAAGATG | GMKGERRRGKG |
| | GCCGAGGCCTACAGCGAGATCGGAATGAAGGGC | HDGLYQGLSTAT |
| | GAGCGCAGAAGAGGCAAGGGACACGATGGACT | KDTYDALHMQA |
| | GTACCAGGGACTGAGCACCGCCACCAAGGATAC | LPPR (SEQ ID |
| | CTATGACGCCCTGCACATGCAGGCCCTGCCTCCA | NO: 334) |
| | AGA (SEQ ID NO: 318) | |
| 9-20 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGTCTTGTGCCGCC | GSLRLSCAASESI |
| | AGCGAGAGCATCAGCAGCATCCACATCATGGCC | SSIHIMA WYRQA |
| | TGGTACAGACAGGCCCCTGGCAAGCAGAGAGAA | PGKQRELVAGIR |
| | CTGGTTGCCGGCATCAGAAACGACGGCAGCACA | NDGSTVYVDSV |
| | GTGTACGTGGACAGCGTGAAGGGCAGATTCACC | KGRFTISRDNAK |
| | ATCAGCCGGGACAACGCCAAGAACAGCGTGTAC | NSVYLQMNSLR |
| | CTGCAGATGAACAGCCTGAGAGCCGAGGACACC | AEDTAVYYCNA |
| | GCCGTGTACTACTGCAATGCCGATCAAGGCTTC | DQGFGSYSEWE |
| | GGCAGCTACAGCGAGTGGGAGAGAAGATCCAG | RRSRWGQGTTV |
| | ATGGGGCCAGGGCACCACCGTGACAGTGTCTAG | TVSSIEVMYPPP |
| | CATCGAAGTGATGTACCCTCCACCTTACCTGGAC | YLDNEKSNGTII |
| | AACGAGAAGTCCAACGGCACCATCATCCACGTG | HVKGKHLCPSPL |
| | AAGGGAAAGCACCTGTGTCCTTCTCCACTGTTCC | FPGPSKPFWVLV |
| | CCGGACCTAGCAAGCCTTTCTGGGTGCTCGTTGT | VVGGVLACYSL |
| | TGTTGGCGGCGTGCTGGCCTGTTACTCTCTGCTG | LVTVAFIIFWVRS |
| | GTTACCGTGGCCTTCATCATCTTTTGGGTCCGAA | KRSRLLHSDYM |
| | GCAAGCGGAGCCGGCTGCTGCACAGCGACTACA | NMTPRRPGPTRK |
| | TGAACATGACCCCTAGACGGCCCGGACCAACCA | HYQPYAPPRDFA |
| | GAAAGCACTACCAGCCTTACGCTCCTCCTAGAG | AYRSRVKFSRSA |
| | ACTTCGCCGCCTACCGGTCCAGAGTGAAGTTCA | DAPAYQQGQNQ |
| | GCAGATCCGCTGATGCCCCTGCCTATCAGCAGG | LYNELNLGRREE |
| | GCCAGAACCAGCTGTACAACGAGCTGAACCTGG | YDVLDKRRGRD |
| | GGAGAAGAGAAGAGTACGACGTGCTGGACAAG | PEMGGKPRRKN |
| | CGGAGAGGCAGAGATCCTGAGATGGGCGGCAA | PQEGLYNELQKD |
| | GCCCAGACGGAAGAATCCTCAAGAGGGCCTGTA | KMAEAYSEIGM |
| | TAATGAGCTGCAGAAAGACAAGATGGCCGAGGC | KGERRRGKGHD |
| | CTACTCCGAGATCGGAATGAAGGGCGAGCGCAG | GLYQGLSTATKD |
| | AAGAGGCAAGGGACACGATGGACTGTATCAGG | TYDALHMQALP |
| | GCCTGAGCACCGCCACCAAGGATACCTATGATG | PR (SEQ ID NO: |
| | CCCTGCACATGCAGGCCCTGCCTCCAAGA (SEQ | 335) |
| | ID NO: 319) | |
| 9-21 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGAGCTGTTCTGCC | GSLRLSCSASGF |
| | AGCGGCTTCACCCTGGACTACTACATGATCGGCT | TLDYYMIGWFR |
| | GGTTCAGACAGGCCCCTGGCAAAGAGAGAGAG | QAPGKEREGLSS |
| | GGCCTGAGCAGCATCTCTCCTGCCGATGGCAGC | ISPADGSTYYAD |
| | ACCTACTACGCCGATTCTGTGAAGGGCAGATTC | SVKGRFTISRDSS |
| | ACCATCAGCCGGGACAGCAGCAAGAACACCGTG | KNTVYLQMNSL |
| | TACCTGCAGATGAACAGCCTGAGAGCCGAGGAC | RAEDTAVYYCA |
| | ACCGCCGTGTACTATTGTGCCGCCGGAAATGAG | AGNEATISWGFG |
| | GCCACAATCAGCTGGGGATTTGGCCCCTGGGAG | PWEYDYWGQGT |
| | TACGATTATTGGGGCCAGGGCACCATGGTCACC | MVTVSSTTTPAP |
| | GTGTCCTCTACAACAACCCCTGCTCCTCGGCCTC | RPPTPAPTIASQP |
| | CTACACCAGCTCCTACAATTGCCAGCCAGCCACT | LSLRPEACRPAA |
| | GTCTCTGAGGCCCGAAGCTTGTAGACCTGCTGCT | GGAVHTRGLDF |
| | GGCGGAGCCGTGCATACAAGAGGACTGGATTTC | ACDIYIWAPLAG |
| | GCCTGCGACATCTACATCTGGGCCCCTCTGGCTG | TCGVLLLSLVITL |
| | GAACATGTGGCGTGCTGCTGCTGAGCCTGGTCA | YCKRGRKKLLYI |
| | TCACCCTGTACTGCAAGCGGGGCAGAAAGAAGC | FKQPFMRPVQTT |
| | TGCTGTACATCTTCAAGCAGCCCTTCATGCGGCC | QEEDGCSCRFPE |
| | CGTGCAGACCACACAAGAGGAAGATGGCTGCTC | EEEGGCELRVKF |
| | CTGCAGATTCCCCGAGGAAGAAGAAGGCGGCTG | SRSADAPAYQQ |
| | CGAGCTGAGAGTGAAGTTCAGCAGATCCGCTGA | GQNQLYNELNL |
| | CGCCCCTGCCTATCAGCAGGGCCAGAACCAGCT | GRREEYDVLDK |
| | GTACAACGAGCTGAACCTGGGGAGAAGAGAAG | RRGRDPEMGGK |
| | AGTACGACGTGCTGGACAAGCGGAGAGGCAGA | PRRKNPQEGLYN |
| | GATCCTGAGATGGGCGGCAAGCCCAGACGGAAG | ELQKDKMAEAY |
| | AATCCTCAAGAGGGACTGTATAATGAGCTGCAG | SEIGMKGERRRG |
| | AAAGACAAGATGGCCGAGGCCTACAGCGAGATC | KGHDGLYQGLS |
| | GGAATGAAGGGCGAGCGCAGAAGAGGCAAGGG | TATKDTYDALH |
| | ACACGATGGACTGTACCAGGGACTGAGCACCGC | MQALPPR (SEQ |
| | CACCAAGGATACCTATGACGCCCTGCACATGCA | ID NO: 336) |
| | GGCCCTGCCTCCAAGA (SEQ ID NO: 320) | |
| 9-22 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGAGCTGTTCTGCC | GSLRLSCSASGF |
| | AGCGGCTTCACCCTGGACTACTACATGATCGGCT | TLDYYMIGWFR |
| | GGTTCAGACAGGCCCCTGGCAAAGAGAGAGAG | QAPGKEREGLSS |
| | GGCCTGAGCAGCATCTCTCCTGCCGATGGCAGC | ISPADGSTYYAD |
| | ACCTACTACGCCGATTCTGTGAAGGGCAGATTC | SVKGRFTISRDSS |
| | ACCATCAGCCGGGACAGCAGCAAGAACACCGTG | KNTVYLQMNSL |
| | TACCTGCAGATGAACAGCCTGAGAGCCGAGGAC | RAEDTAVYYCA |
| | ACCGCCGTGTACTATTGTGCCGCCGGAAATGAG | AGNEATISWGFG |
| | GCCACAATCAGCTGGGGATTTGGCCCCTGGGAG | PWEYDYWGQGT |
| | TACGATTATTGGGGCCAGGGCACCATGGTCACA | MVTVSSGGGGS |
| | GTGTCTAGTGGCGGCGGAGGCAGTGAAGTCCAG | EVQLVESGGGLV |
| | CTTGTTGAAAGCGGAGGCGGCCTTGTGCAACCT | QPGGSLRLSCAA |
| | GGTGGCTCTCTCAGACTGTCCTGTGCCGCCAGCG | SESISSIHIMAWY |
| | AGAGCATCAGCAGCATCCACATCATGGCCTGGT | RQAPGKQRELV |
| | ATAGGCAGGCTCCAGGCAAGCAGAGAGAGCTG | AGIRNDGSTVYV |
| | GTTGCCGGCATCAGAAACGATGGCTCTACCGTG | DSVKGRFTISRD |
| | TACGTGGACAGCGTGAAAGGCCGGTTCACCATC | NAKNSVYLQMN |
| | TCCAGAGACAACGCCAAGAATTCTGTGTATCTC | SLRAEDTAVYYC |
| | CAGATGAATTCCCTGCGCGCCGAAGATACAGCC | NADQGFGSYSE |
| | GTGTATTACTGCAACGCCGACCAAGGCTTCGGC | WERRSRWGQGT |
| | AGCTACAGCGAATGGGAGAGAAGATCCAGATG | TVTVSSIEVMYP |
| | GGGACAGGGAACCACCGTGACCGTGTCCAGCAT | PPYLDNEKSNGT |
| | CGAAGTGATGTACCCTCCACCTTACCTGGACAA | IIHVKGKHLCPSP |
| | CGAGAAGTCCAACGGCACCATCATCCACGTGAA | LFPGPSKPFWVL |
| | GGGAAAGCACCTGTGTCCTTCTCCACTGTTCCCC | VVVGGVLACYS |
| | GGACCTAGCAAGCCTTTCTGGGTGCTCGTTGTTG | LLVTVAFIIFWV |
| | TTGGCGGCGTGCTGGCCTGTTACTCTCTGCTGGT | RSKRSRLLHSDY |
| | TACCGTGGCCTTCATCATCTTTTGGGTCCGAAGC | MNMTPRRPGPT |
| | AAGCGGAGCCGGCTGCTGCACAGCGACTACATG | RKHYQPYAPPRD |
| | AACATGACCCCTAGACGGCCCGGACCAACCAGA | FAAYRSRVKFSR |
| | AAGCACTACCAGCCTTACGCTCCTCCTAGAGACT | SADAPAYQQGQ |
| | TCGCCGCCTACCGGTCCAGAGTGAAGTTCAGCA | NQLYNELNLGR |
| | GATCCGCTGATGCCCCTGCCTATCAGCAGGGCC | REEYDVLDKRR |
| | AGAACCAGCTGTACAACGAGCTGAACCTGGGGA | GRDPEMGGKPR |
| | GAAGAGAAGAGTACGACGTGCTGGACAAGCGG | RKNPQEGLYNEL |
| | AGAGGCAGAGATCCTGAGATGGGCGGCAAGCCC | QKDKMAEAYSEI |
| | AGACGGAAGAATCCTCAAGAGGGACTGTATAAT | GMKGERRRGKG |
| | GAGCTGCAGAAAGACAAGATGGCCGAGGCCTAC | HDGLYQGLSTAT |
| | AGCGAGATCGGAATGAAGGGCGAGCGCAGAAG | KDTYDALHMQA |
| | AGGCAAGGGACACGATGGACTGTACCAGGGACT | LPPR (SEQ ID |
| | GAGCACCGCCACCAAGGATACCTATGACGCCCT | NO: 337) |
| | GCACATGCAGGCCCTGCCTCCAAGA (SEQ ID NO: | |
| | 321) | |
| 9-23 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGAGCTGTTCTGCC | GSLRLSCSASGF |
| | AGCGGCTTCACCCTGGACTACTACATGATCGGCT | TLDYYMIGWFR |
| | GGTTCAGACAGGCCCCTGGCAAAGAGAGAGAG | QAPGKEREGLSS |
| | GGCCTGAGCAGCATCTCTCCTGCCGATGGCAGC | ISPADGSTYYAD |
| | ACCTACTACGCCGATTCTGTGAAGGGCAGATTC | SVKGRFTISRDSS |
| | ACCATCAGCCGGGACAGCAGCAAGAACACCGTG | KNTVYLQMNSL |
| | TACCTGCAGATGAACAGCCTGAGAGCCGAGGAC | RAEDTAVYYCA |
| | ACCGCCGTGTACTATTGTGCCGCCGGAAATGAG | AGNEATISWGFG |
| | GCCACAATCAGCTGGGGATTTGGCCCCTGGGAG | PWEYDYWGQGT |
| | TACGATTATTGGGGCCAGGGCACCATGGTCACA | MVTVSSGGGGS |
| | GTGTCTAGTGGCGGCGGAGGCAGTGAAGTCCAG | EVQLVESGGGLV |
| | CTTGTTGAAAGCGGAGGCGGCCTTGTGCAACCT | QPGGSLRLSCAA |
| | GGTGGCTCTCTCAGACTGTCCTGTGCCGCCAGCG | SESISSIHIMAWY |
| | AGAGCATCAGCAGCATCCACATCATGGCCTGGT | RQAPGKQRELV |
| | ATAGGCAGGCTCCAGGCAAGCAGAGAGAGCTG | AGIRNDGSTVYV |
| | GTTGCCGGCATCAGAAACGATGGCTCTACCGTG | DSVKGRFTISRD |
| | TACGTGGACAGCGTGAAAGGCCGGTTCACCATC | NAKNSVYLQMN |
| | TCCAGAGACAACGCCAAGAATTCTGTGTATCTC | SLRAEDTAVYYC |
| | CAGATGAATTCCCTGCGCGCCGAAGATACAGCC | NADQGFGSYSE |
| | GTGTATTACTGCAACGCCGACCAAGGCTTCGGC | WERRSRWGQGT |
| | AGCTACAGCGAATGGGAGAGAAGATCCAGATG | TVTVSSTTTPAP |
| | GGGACAGGGAACCACCGTGACCGTGTCCAGCAC | RPPTPAPTIASQP |
| | AACAACCCCTGCTCCTAGACCTCCTACACCAGCT | LSLRPEACRPAA |
| | CCTACAATCGCCAGCCAGCCTCTGTCTCTGAGGC | GGAVHTRGLDF |
| | CAGAAGCTTGTAGACCTGCTGCTGGCGGAGCCG | ACDIYIWAPLAG |
| | TGCATACAAGAGGACTGGATTTCGCCTGCGACA | TCGVLLLSLVITL |
| | TCTACATCTGGGCCCCTCTGGCTGGAACATGTGG | YCKRGRKKLLYI |
| | CGTGCTGCTGCTGAGCCTGGTCATCACCCTGTAC | FKQPFMRPVQTT |
| | TGCAAGCGGGGCAGAAAGAAGCTGCTGTACATC | QEEDGCSCRFPE |
| | TTCAAGCAGCCCTTCATGCGGCCCGTGCAGACC | EEEGGCELR VKF |
| | ACACAAGAGGAAGATGGCTGCTCCTGCAGATTC | SRSADAPAYQQ |
| | CCCGAGGAAGAAGAAGGCGGCTGCGAGCTGAG | GQNQLYNELNL |
| | AGTGAAGTTCAGCAGATCCGCTGACGCCCCTGC | GRREEYDVLDK |
| | CTATCAGCAGGGCCAGAACCAGCTGTACAACGA | RRGRDPEMGGK |
| | GCTGAACCTGGGGAGAAGAGAAGAGTACGACG | PRRKNPQEGLYN |
| | TGCTGGATAAGCGGAGAGGCAGAGATCCTGAGA | ELQKDKMAEAY |
| | TGGGCGGCAAGCCCAGACGGAAGAATCCTCAAG | SEIGMKGERRRG |
| | AGGGACTGTATAATGAGCTGCAGAAAGACAAGA | KGHDGLYQGLS |
| | TGGCCGAGGCCTACAGCGAGATCGGAATGAAGG | TATKDTYDALH |
| | GCGAGCGCAGAAGAGGCAAGGGACACGATGGA | MQALPPR (SEQ |
| | CTGTACCAGGGACTGAGCACCGCCACCAAGGAT | ID NO: 338) |
| | ACCTATGACGCCCTGCACATGCAGGCCCTGCCTC | |
| | CAAGA (SEQ ID NO: 322) | |
| 9-24 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | GCAGCTGGTTGAATCTGGCGGAGGACTGGTTCA | LVESGGGLVQPG |
| | GCCTGGCGGATCTCTGAGACTGTCTTGTGCCGCC | GSLRLSCAASESI |
| | AGCGAGAGCATCAGCAGCATCCACATCATGGCC | SSIHIMA WYRQA |
| | TGGTACAGACAGGCCCCTGGCAAGCAGAGAGAA | PGKQRELVAGIR |
| | CTGGTTGCCGGCATCAGAAACGACGGCAGCACA | NDGSTVYVDSV |
| | GTGTACGTGGACAGCGTGAAGGGCAGATTCACC | KGRFTISRDNAK |
| | ATCAGCCGGGACAACGCCAAGAACAGCGTGTAC | NSVYLQMNSLR |
| | CTGCAGATGAACAGCCTGAGAGCCGAGGACACC | AEDTAVYYCNA |
| | GCCGTGTACTACTGCAATGCCGATCAAGGCTTC | DQGFGSYSEWE |
| | GGCAGCTACAGCGAGTGGGAGAGAAGATCCAG | RRSRWGQGTTV |
| | ATGGGGCCAGGGCACCACCGTGACAGTGTCTAG | TVSSTTTPAPRPP |
| | CACAACAACCCCTGCTCCTCGGCCTCCTACACCA | TPAPTIASQPLSL |
| | GCTCCTACAATTGCCAGCCAGCCACTGTCTCTGA | RPEACRPAAGGA |
| | GGCCCGAAGCTTGTAGACCTGCTGCTGGCGGAG | VHTRGLDFACDI |
| | CCGTGCATACAAGAGGACTGGATTTCGCCTGCG | YIWAPLAGTCGV |
| | ACATCTACATCTGGGCCCCTCTGGCTGGAACATG | LLLSLVITLYCKR |
| | TGGCGTGCTGCTGCTGAGCCTGGTCATCACCCTG | GRKKLLYIFKQP |
| | TACTGCAAGCGGGGCAGAAAGAAGCTGCTGTAC | FMRPVQTTQEED |
| | ATCTTCAAGCAGCCCTTCATGCGGCCCGTGCAG | GCSCRFPEEEEG |
| | ACCACACAAGAGGAAGATGGCTGCTCCTGCAGA | GCELRVKFSRSA |
| | TTCCCCGAGGAAGAAGAAGGCGGCTGCGAGCTG | DAPAYQQGQNQ |
| | AGAGTGAAGTTCAGCAGATCCGCTGACGCCCCT | LYNELNLGRREE |
| | GCCTATCAGCAGGGACAGAACCAGCTGTACAAC | YDVLDKRRGRD |
| | GAGCTGAACCTGGGGAGAAGAGAAGAGTACGA | PEMGGKPRRKN |
| | CGTGCTGGACAAGCGGAGAGGCAGAGATCCTGA | PQEGLYNELQKD |
| | GATGGGCGGCAAGCCCAGACGGAAGAATCCTCA | KMAEAYSEIGM |
| | AGAGGGCCTGTATAATGAGCTGCAGAAAGACAA | KGERRRGKGHD |
| | GATGGCCGAGGCCTACTCCGAGATCGGAATGAA | GLYQGLSTATKD |
| | GGGCGAGCGCAGAAGAGGCAAGGGACACGATG | TYDALHMQALP |
| | GACTGTATCAGGGCCTGAGCACCGCCACCAAGG | PR (SEQ ID NO: |
| | ATACCTATGATGCCCTGCACATGCAGGCCCTGCC | 339) |
| | TCCAAGA (SEQ ID NO: 323) | |
| 9-25 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | TCAGCTGCAAGCTTCTGGCGGAGGACTTGCTCA | LQASGGGLAQP |
| | ACCTGGCGGAAGCCTGAGACTGTCTTGTGCCGC | GGSLRLSCAASG |
| | CTCTGGCAGAACCTTCAGCACCTACTTCATGGCC | RTFSTYFMAWFR |
| | TGGTTCAGACAGCCTCCTGGCAAAGGCCTGGAA | QPPGKGLEYVG |
| | TACGTTGGCGGAATCCGTTGGAGTGATGGCGTG | GIRWSDGVPHY |
| | CCACACTACGCCGATAGCGTGAAGGGCAGATTC | ADSVKGRFTISR |
| | ACCATCAGCCGGGACAACGCCAAGAACACCGTG | DNAKNTVYLQM |
| | TACCTGCAGATGAACAGCCTGAGAGCCGAGGAT | NSLRAEDTAVYF |
| | ACCGCCGTGTACTTCTGTGCCAGCAGAGGAATC | CASRGIADGSDF |
| | GCCGACGGCAGCGATTTTGGCTCTTATGGCCAG | GSYGQGTQVTV |
| | GGCACCCAAGTGACCGTTAGCTCTCCTGCCAAG | SSPAKPTTTPAPR |
| | CCTACCACCACACCAGCTCCTAGACCTCCAACTC | PPTPAPTIASQPL |
| | CTGCTCCTACAATCGCCAGCCAGCCTCTGTCTCT | SLRPEACRPAAG |
| | GAGGCCAGAAGCCTGTAGACCTGCTGCTGGCGG | GAVHTRGLDFA |
| | AGCCGTGCATACAAGAGGACTGGATTTCGCCTG | CDIYIWAPLAGT |
| | CGACATCTACATCTGGGCCCCTCTGGCTGGAAC | CGVLLLSL VITL |
| | ATGTGGCGTGCTGCTGCTGAGCCTGGTCATCACC | YCKRGRKKLLYI |
| | CTGTACTGCAAGCGGGGCAGAAAGAAGCTGCTG | FKQPFMRPVQTT |
| | TACATCTTCAAGCAGCCCTTCATGCGGCCCGTGC | QEEDGCSCRFPE |
| | AGACCACACAAGAGGAAGATGGCTGCTCCTGCA | EEEGGCELRVKF |
| | GATTCCCCGAGGAAGAAGAAGGCGGCTGCGAGC | SRSADAPAYQQ |
| | TGAGAGTGAAGTTCAGCAGATCCGCCGACGCTC | GQNQLYNELNL |
| | CTGCCTATCAGCAGGGACAGAACCAGCTGTACA | GRREEYDVLDK |
| | ACGAGCTGAATCTGGGGCGCAGAGAAGAGTACG | RRGRDPEMGGK |
| | ACGTGCTGGACAAGAGAAGAGGCAGGGACCCT | PQRRKNPQEGLY |
| | GAGATGGGCGGAAAGCCCCAGAGAAGAAAGAA | NELQKDKMAEA |
| | CCCTCAAGAGGGCCTGTATAATGAGCTGCAGAA | YSEIGMKGERRR |
| | AGACAAGATGGCCGAGGCCTACAGCGAGATCGG | GKGHDGLYQGL |
| | AATGAAGGGCGAACGCAGAAGAGGAAAGGGCC | STATKDTYDALH |
| | ACGACGGACTGTATCAGGGCCTGAGCACAGCCA | MQALPPR (SEQ |
| | CCAAGGACACCTATGATGCCCTGCACATGCAGG | ID NO: 340) |
| | CCCTGCCTCCAAGA (SEQ ID NO: 324) | |
| 9-26 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | TCAGCTGCAAGCTTCTGGCGGAGGACTTGCTCA | LQASGGGLAQP |
| | ACCTGGCGGAAGCCTGAGACTGTCTTGTGCCGC | GGSLRLSCAASG |
| | CTCTGGCAGAACCTTCAGCACCTACTTCATGGCC | RTFSTYFMAWFR |
| | TGGTTCAGACAGCCTCCTGGCAAAGGCCTGGAA | QPPGKGLEYVG |
| | TACGTTGGCGGAATCCGTTGGAGTGATGGCGTG | GIRWSDGVPHY |
| | CCACACTACGCCGATAGCGTGAAGGGCAGATTC | ADSVKGRFTISR |
| | ACCATCAGCCGGGACAACGCCAAGAACACCGTG | DNAKNTVYLQM |
| | TACCTGCAGATGAACAGCCTGAGAGCCGAGGAT | NSLRAEDTAVYF |
| | ACCGCCGTGTACTTCTGTGCCAGCAGAGGAATC | CASRGIADGSDF |
| | GCCGACGGCAGCGATTTTGGCTCTTATGGCCAG | GSYGQGTQVTV |
| | GGCACCCAAGTGACCGTGTCCAGCACAACAACC | SSTTTPAPRPPTP |
| | CCTGCTCCTAGACCTCCTACACCAGCTCCTACAA | APTIASQPLSLRP |
| | TCGCCAGCCAGCCTCTGTCTCTGAGGCCAGAAG | EACRPAAGGAV |
| | CCTGTAGACCTGCTGCTGGCGGAGCCGTGCATA | HTRGLDFACDIY |
| | CAAGAGGACTGGATTTCGCCTGCGACATCTACA | IWAPLAGTCGVL |
| | TCTGGGCCCCTCTGGCTGGAACATGTGGCGTGCT | LLSLVITLYCKR |
| | GCTGCTGAGCCTGGTCATCACCCTGTACTGCAAG | GRKKLLYIFKQP |
| | CGGGGCAGAAAGAAGCTGCTGTACATCTTCAAG | FMRPVQTTQEED |
| | CAGCCCTTCATGCGGCCCGTGCAGACCACACAA | GCSCRFPEEEEG |
| | GAGGAAGATGGCTGCTCCTGCAGATTCCCCGAG | GCELRVKFSRSA |
| | GAAGAAGAAGGCGGCTGCGAGCTGAGAGTGAA | DAPAYQQGQNQ |
| | GTTCAGCAGATCCGCCGACGCTCCTGCCTATCAG | LYNELNLGRREE |
| | CAGGGACAGAACCAGCTGTACAACGAGCTGAAT | YDVLDKRRGRD |
| | CTGGGGCGCAGAGAAGAGTACGACGTGCTGGAT | PEMGGKPQRRK |
| | AAGCGGAGAGGCAGAGATCCTGAGATGGGCGG | NPQEGLYNELQK |
| | AAAGCCCCAGCGGAGAAAGAATCCTCAAGAGG | DKMAEAYSEIG |
| | GCCTGTATAATGAGCTGCAGAAAGACAAGATGG | MKGERRRGKGH |
| | CCGAGGCCTACAGCGAGATCGGAATGAAGGGCG | DGLYQGLSTATK |
| | AACGCAGAAGAGGCAAGGGCCACGATGGACTG | DTYDALHMQAL |
| | TATCAGGGCCTGAGCACCGCCACCAAGGATACC | PPR (SEQ ID NO: |
| | TATGATGCCCTGCACATGCAGGCCCTGCCTCCAA | 341) |
| | GA (SEQ ID NO: 325) | |
| 9-27 | ATGGCTCTGCCTGTGACAGCTCTGCTGCTGCCTC | MALPVTALLLPL |
| | TGGCTCTGCTTCTGCATGCCGCTAGACCTGAGGT | ALLLHAARPEVQ |
| | TCAGCTGCAAGCTTCTGGCGGAGGACTTGCTCA | LQASGGGLAQP |
| | ACCTGGCGGAAGCCTGAGACTGTCTTGTGCCGC | GGSLRLSCAASG |
| | CTCTGGCAGAACCTTCAGCACCTACTTCATGGCC | RTFSTYFMA WFR |
| | TGGTTCAGACAGCCTCCTGGCAAAGGCCTGGAA | QPPGKGLEYVG |
| | TACGTTGGCGGAATCCGTTGGAGTGATGGCGTG | GIRWSDGVPHY |
| | CCACACTACGCCGATAGCGTGAAGGGCAGATTC | ADSVKGRFTISR |
| | ACCATCAGCCGGGACAACGCCAAGAACACCGTG | DNAKNTVYLQM |
| | TACCTGCAGATGAACAGCCTGAGAGCCGAGGAT | NSLRAEDTAVYF |
| | ACCGCCGTGTACTTCTGTGCCAGCAGAGGAATC | CASRGIADGSDF |
| | GCCGACGGCAGCGATTTTGGCTCTTATGGCCAG | GSYGQGTQVTV |
| | GGCACCCAAGTGACCGTGTCCAGCATCGAAGTG | SSIEVMYPPPYL |
| | ATGTACCCTCCACCTTACCTGGACAACGAGAAG | DNEKSNGTIIHV |
| | TCCAACGGCACCATCATCCACGTGAAGGGAAAG | KGKHLCPSPLFP |
| | CACCTGTGTCCTTCTCCACTGTTCCCCGGACCTA | GPSKPFWVLVV |
| | GCAAGCCTTTCTGGGTGCTCGTTGTTGTTGGCGG | VGGVLACYSLL |
| | CGTGCTGGCCTGTTACTCTCTGCTGGTTACCGTG | VTVAFIIFWVRS |
| | GCCTTCATCATCTTTTGGGTCCGAAGCAAGCGGA | KRSRLLHSDYM |
| | GCCGGCTGCTGCACAGCGACTACATGAACATGA | NMTPRRPGPTRK |
| | CCCCTAGACGGCCCGGACCAACCAGAAAGCACT | HYQPYAPPRDFA |
| | ACCAGCCTTACGCTCCTCCTAGAGACTTCGCCGC | AYRSRVKFSRSA |
| | CTACCGGTCCAGAGTGAAGTTCAGCAGATCCGC | DAPAYQQGQNQ |
| | CGATGCTCCCGCCTATCAGCAGGGACAGAACCA | LYNELNLGRREE |
| | GCTGTACAACGAGCTGAATCTGGGGCGCAGAGA | YDVLDKRRGRD |
| | AGAGTACGACGTGCTGGATAAGCGGAGAGGCA | PEMGGKPQRRK |
| | GAGATCCTGAGATGGGCGGAAAGCCCCAGCGGA | NPQEGLYNELQK |
| | GAAAGAATCCTCAAGAGGGCCTGTATAATGAGC | DKMAEAYSEIG |
| | TGCAGAAAGACAAGATGGCCGAGGCCTACAGCG | MKGERRRGKGH |
| | AGATCGGAATGAAGGGCGAACGCAGAAGAGGC | DGLYQGLSTATK |
| | AAGGGCCACGATGGACTGTATCAGGGCCTGAGC | DTYDALHMQAL |
| | ACCGCCACCAAGGATACCTATGATGCCCTGCAC | PPR (SEQ ID NO: |
| | ATGCAGGCCCTGCCTCCAAGA (SEQ ID NO: 326) | 342) |

TABLE 10
Exemplary Binders
Where applicable, CDR sequences are bolded
and underlined and/or known in the art.

| ID | Exemplary binding molecule and amino acid sequence |
|---|---|
| 10-1 | Heavy |
| | QVQLQQPGAELVKPGASVKMSCKASGYTFTSYNMHWVKQTPGRGLEW |
| | IGAIYPGNGDTSYNQKFKGKATLTADKSSSTAYMQLSSLTSEDSAVYYC |
| | ARSTYYGGDWYFNVWGAGTTVTVSAASTKGPSVFPLAPSSKSTSGGTA |
| | ALGCLVKDYFPEPVTVSWNSGALTSGVHTFPAVLQSSGLYSLSSVVTVP |
| | SSSLGTQTYICNVNHKPSNTKVDKKAEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALH |
| | NHYTQKSLSLSPGK (SEQ ID NO: 343) |
| | Light |
| | QIVLSQSPAILSASPGEKVTMTCRASSSVSYIHWFQQKPGSSPKPWIYATS |
| | NLASGVPVRFSGSGSGTSYSLTISRVEAEDAATYYCQQWTSNPPTFGGG |
| | TKLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 344) |
| 10-2 | Heavy |
| | QAYLQQSGAELVRPGASVKMSCKASGYTFTSYNMHWVKQTPRQGLEW |
| | IGAIYPGNGDTSYNQKFKGKATLTVDKSSSTAYMQLSSLTSEDSAVYFC |
| | ARVVYYSNSYWYFDVWGTGTTVTVSAPSVYPLAPVCGDTTGSSVTLGC |
| | LVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNLLGGPSVFIFPPK |
| | IKDVLMISLSPIVTCVVVDVSEDDPDVQISWFVNNVEVHTAQTQTHRED |
| | YNSTLRVVSALPIQHQDWMSGKEFKCKVNNKDLPAPIERTISKPKGSVR |
| | APQVYVLPPPEEEMTKKQVTLTCMVTDFMPEDIYVEWTNNGKTELNYK |
| | NTEPVLDSDGSYFMYSKLRVEKKNWVERNSYSCSVVHEGLHNHHTTKS |
| | FSR (SEQ ID NO: 345) |
| | Light |
| | QIVLSQSPAILSASPGEKVTMTCRASSSVSYMHWYQQKPGSSPKPWIYAP |
| | SNLASGVPARFSGSGSGTSYSLTISRVEAEDAATYYCQQWSFNPPTFGAG |
| | TKLELKRADAAPTVFIFPPSDEQLKSGTASVVCLLNNFYPREAKVQWKV |
| | DNALQSGNSQESVTEQDSKDSTYSLSSTLTLSKADYEKHKVYACEVTHQ |
| | GLSSPVTKSFN (SEQ ID NO: 346) |
| 10-3 | Heavy |
| | EVQLVESGGGLVQPGGSLRLSCAASGYTFTSYNMHWVRQAPGKGLEW |
| | VGAIYPGNGDTSYNQKFKGRFTISVDKSKNTLYLQMNSLRAEDTAVYY |
| | CARVVYYSNSYWYFDVWGQGTLVTVSSASTKGPSVFPLAPSSKSTSGGT |
| | AALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALH |
| | NHYTQKSLSLSPGK (SEQ ID NO: 347) |
| | Light |
| | DIQMTQSPSSLSASVGDRVTITCRASSSVSYMHWYQQKPGKAPKPLIYAP |
| | SNLASGVPSRFSGSGSGTDFTLTISSLQPEDFATYYCQQWSFNPPTFGQGT |
| | KVEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 348) |
| 10-4 | Heavy |
| | QVQLQQSGAEVKKPGSSVKVSCKASGYTFTSYNMHWVKQAPGQGLEW |
| | IGAIYPGNGDTSYNQKFKGKATLTADESTNTAYMELSSLRSEDTAFYYC |
| | ARSTYYGGDWYFDVWGQGTTVTVSSASTKGPSVFPLAPSSKSTSGGTA |
| | ALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPELLGGPS |
| | VFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALH |
| | NHYTQKSLSLSPGK (SEQ ID NO: 349) |
| | Light |
| | DIQLTQSPSSLSASVGDRVTMTCRASSSVSYIHWFQQKPGKAPKPWIYAT |
| | SNLASGVPVRESGSGSGTDYTFTISSLQPEDIATYYCQQWTSNPPTFGGG |
| | TKLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 350) |
| 10-5 | Heavy |
| | EVQLVESGGGLVQPGRSLRLSCAASGFTFNDYAMHWVRQAPGKGLEW |
| | VSTISWNSGSIGYADSVKGRFTISRDNAKKSLYLQMNSLRAEDTALYYC |
| | AKDIQYGNYYYGMDVWGQGTTVTVSSASTKGPSVFPLAPGSSKSTSGT |
| | AALGCLVKDYFPEPVTVSWNSGALTSGVHTFPAVLQSSGLYSLSSVVTV |
| | PSSSLGTQTYICNVNHKPSNTKVDKKVEP (SEQ ID NO: 351) |
| | Light |
| | EIVLTQSPATLSLSPGERATLSCRASQSVSSYLAWYQQKPGQAPRLLIYD |
| | ASNRATGIPARFSGSGSGTDFTLTISSLEPEDFAVYYCQQRSNWPITFGQG |
| | TRLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNR (SEQ ID NO: 352) |
| 10-6 | Heavy |
| | QAYLQQSGAELVRPGASVKMSCKASGYTFTSYNMHWVKQTPRQGLEW |
| | IGGIYPGNGDTSYNQKFKGKATLTVGKSSSTAYMQLSSLTSEDSAVYFC |
| | ARYDYNYAMDYWGQGTSVTVSSASTKGPSVFPLAPSSKSTSGGTAALG |
| | CLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYT |
| | QKSLSLSPGK (SEQ ID NO: 353) |
| | Light |
| | QIVLSQSPAILSASPGEKVTMTCRASSSVSYMHWYQQKPGSSPKPWIYAT |
| | SNLASGVPARFSGSGSGTSYSFTISRVEAEDAATYYCQQWTFNPPTFGGG |
| | TRLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 354) |
| 10-7 | Heavy |
| | EVQLVESGGGLVQPDRSLRLSCAASGFTFHDYAMHWVRQAPGKGLEW |
| | VSTISWNSGTIGYADSVKGRFTISRDNAKNSLYLQMNSLRAEDTALYYC |
| | AKDIQYGNYYYGMDVWGQGTTVTVSSASTKGPSVFPLAPSSKSTSGGT |
| | AALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPEFEGGP |
| | SVFLFPPKPKDTLMISRTPEVTCVVVAVSHEDPEVKFNWYVDGVEVHNA |
| | KTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNG |
| | QPENNYKTTPPVLDSDGSFFLYSRLTVDKSRWQQGNVFSCSVMHEALH |
| | NHYTQKSLSLSPG (SEQ ID NO: 355) |
| | Light |
| | EIVLTQSPATLSLSPGERATLSCRASQSVSSYLAWYQQKPGQAPRLLIYD |
| | ASNRATGIPARFSGSGSGTDFTLTISSLEPEDFAVYYCQQRSNWPITFGQG |
| | TRLEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 356) |
| 10-8 | Heavy |
| | QAYLQQSGAELVRPGASVKMSCKASGYTFTSYNMHWVKQTPRQGLEW |
| | IGAIYPGNGDTSYNQKFKGKATLTVDKSSSTAYMQLSSLTSEDSAVYFC |
| | ARVVYYSNSYWYFDVWGTGTTVTVSGPSVFPLAPSSKSTSGGTAALGC |
| | LVKDYFPEPVTVSWNSGALTSGVHTFPAVLQSSGLYSLSSVVTVPSSSLG |
| | TQTYICNVNHKPSNTKVDKKAEPKSCDKTHTCPPCPAPELLGGPSVFLFP |
| | PKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYT |
| | QKSLSLSPGK (SEQ ID NO: 357) |
| | Light |
| | QIVLSQSPAILSASPGEKVTMTCRASSSVSYMHWYQQKPGSSPKPWIYAP |
| | SNLASGVPARFSGSGSGTSYSLTISRVEAEDAATYYCQQWSFNPPTFGAG |
| | TKLELKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQ |
| | GLSSPVTKSFNR (SEQ ID NO: 358) |
| 10-9 | Heavy |
| | EVQLVESGGGLVQPGGSLRLSCAASGYTFTSYNMHWVRQAPGKGLEW |
| | VGAIYPGNGDTSYNQKFKGRFTISVDKSKNTLYLQMNSLRAEDTAVYY |
| | CARVVYYSNSYWYFDVWGQGTLVTVSSASTKGPSVFPLAPSSKSTSGGT |
| | AALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNA |
| | KTKPREEQYGSTYRVVSVLTVLHQDWLNGKEYKCKVSNKALPAPIEKTI |
| | SKAKGQPREPQVYTLPPSREEMTKNQVSLWCLVKGFYPSDIAVEWESN |
| | GQPENNYKTTPPVLDSDGSFFLYSKLTVDKSRWQQGNVFSCSVMHEAL |
| | HNHYTQKSLSLSPGK (SEQ ID NO: 359) |
| | Light |
| | DIQMTQSPSSLSASVGDRVTITCRASSSVSYMHWYQQKPGKAPKPLIYAP |
| | SNLASGVPSRFSGSGSGTDFTLTISSLQPEDFATYYCQQWSFNPPTFGQGT |
| | KVEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQG |
| | LSSPVTKSFNRGEC (SEQ ID NO: 348) |
| 10-10 | Heavy |
| | EVQLQQSGAELVKPGASVKMSCKASGYTFTSYNMHWVKQTPGQGLEW |
| | IGAIYPGNGDTSYNQKFKGKATLTADKSSSTAYMQLSSLTSEDSADYYC |
| | ARSNYYGSSYWFFDVWGAGTTVTVSS (SEQ ID NO: 360) |
| | Light |
| | DIVLTQSPAILSASPGEKVTMTCRASSSVNYMDWYQKKPGSSPKPWIYA |
| | TSNLASGVPARFSGSGSGTSYSLTISRVEAEDAATYYCQQWSFNPPTFGG |
| | GTKLEIK (SEQ ID NO: 361) |
| 10-11 | Heavy |
| | EVQLQQSGAELKKPGASVKVSCKASGYTFTSYNMHWVKQTPGQGLEW |
| | IGAIYPGNGDTSYNQKFKGKTTLTADKSSSTAYMELSSLRSEDTAVYYC |
| | ARSNYYGSSYWFFDVWGTGTTVTVSS (SEQ ID NO: 362) |
| | Light |
| | DIVLTQSPAIITASPGEKVTMTCRASSSVNYMDWYQKKPGSSPKPWIYAT |
| | SNLASGVPSRFSGSGSGTTYSMTISSLEAEDAATYYCQQWSFNPPTFGGG |
| | TKLEIK (SEQ ID NO: 363) |
| 10-12 | scFv |
| | QVQLQQPGAELVKPGASVKMSCKASGYTFTSYNMHWVKQTPGRGLEW |
| | IGAIYPGNGDTSYNQKFKGKATLTADKSSSTAYMQLSSLTSEDSAVYYC |
| | ARSTYYGGDWYFNVWGAGTTVTVSAGGGGSGGGGSGGGGSQIVLSQS |
| | PAILSASPGEKVTMTCRASSSVSYIHWFQQKPGSSPKPWIYATSNLASGV |
| | PVRFSGSGSGTSYSLTISRVEAEDAATYYCQQWTSNPPTFGGGTKLEIKR |
| | TG (SEQ ID NO: 364) |
| 10-13 | scFv |
| | QVQLQQPGAELVKPGASVKMSCKASGYTFTSYNMHWVKQTPGRGLEW |
| | IGAIYPGNGDTSYNQKFKGKATLTADKSSSTAYMQLSSLTSEDSAVYYC |
| | ARSTYYGGDWYFNVWGAGTTVTVSAGGGGSGGGGSGGGGSGGGGSQI |
| | VLSQSPAILSASPGEKVTMTCRASSSVSYIHWFQQKPGSSPKPWIYATSN |
| | LASGVPVRFSGSGSGTSYSLTISRVEAEDAATYYCQQWTSNPPTFGGGT |
| | GLEIGRTG (SEQ ID NO: 365) |
| 10-14 | scFv |
| | DIVMTQTPLSSPVTLGQPASISCRSSQSLVYSDGNTYLSWLQQRPGQPPR |
| | LLIYKISNRFSGVPDRFSGSGAGTDFTLKISRVEAEDVGVYYCVQATQFP |
| | LTFGGGTKVEIKGGGGSGGGGSGGGGSEVQLVQSGAEVKKPGESLKISC |
| | KGSGYSFTSYWIGWVRQMPGKGLEWMGIIYPGDSDTRYSPSFQGQVTIS |
| | ADKSITTAYLQWSSLKASDTAMYYCARHPSYGSGSPNFDYWGQGTLVT |
| | VSS (SEQ ID NO: 366) |
| 10-15 | scFv |
| | DIVLTQSPAILSASPGEKVTMTCRASSSVNYMDWYQKKPGSSPKPWIYA |
| | TSNLASGVPARFSGSGSGTSYSLTISRVEAEDAATYYCQQWSFNPPTFGG |
| | GTKLEIKGSTSGGGSGGGSGGGGSSEVQLQQSGAELVKPGASVKMSCK |
| | ASGYTFTSYNMHWVKQTPGQGLEWIGAIYPGNGDTSYNQKFKGKATLT |
| | ADKSSSTAYMQLSSLTSEDSADYYCARSNYYGSSYWFFDVWGAGTTVT |
| | VSSLDESKY (SEQ ID NO: 367) |
| 10-16 | scFv |
| | DIVLSQSPAILSASPGEKVTMTCRASSSLSFMHWYQQKPGSSPKPWIYAT |
| | SNLASGVPARFSGSGSGTSYSLTISRVEAEDAATYFCHQWSSNPLTFGAG |
| | TKLELKGSTSGGGSGGGSGGGGSSQVQLRQPGAELVKPGASVKMSCKA |
| | SGYTFTSYNMHWVKQTPGQGLEWIGAIYPGNGDTSYNQKFKGKATLTA |
| | DKSSSTAYMQLSSLTSEDSAVYYCARSHYGSNYVDYFDYWGQGTTLTV |
| | SSLDPKSS (SEQ ID NO: 368) |
| 10-17 | Heavy |
| | EVKLQESGPGLVAPSQSLSVTCTVSGVSLPDYGVSWIRQPPRKGLEWLG |
| | VIWGSETTYYNSALKSRLTIIKDNSKSQVFLKMNSLQTDDTAIYYCAKH |
| | YYYGGSYAMDYWGQGTSVTVSS (SEQ ID NO: 369) |
| | Light |
| | DIQMTQTTSSLSASLGDRVTISCRASQDISKYLNWYQQKPDGTVKLLIYH |
| | TSRLHSGVPSRFSGSGSGTDYSLTISNLEQEDIATYFCQQGNTLPYTFGGG |
| | TKLEIT (SEQ ID NO: 370) |
| 10-18 | Heavy |
| | EVQLQQSGAEVKKPGASVKVSCKASGYTFTNYWMHWVRQRPGQGLE |
| | WMGEIDPSDNYANYNQEFQGRVTITVDKSASTAYMELSSLRSEDTAVY |
| | YCARHDGYFGALDYWGQGTTVTVSS (SEQ ID NO: 371) |
| | Light |
| | EIVLTQSPATLSLSPGERATLSCSASSSISSNYLHWYQQKPGQAPRFLIYR |
| | TSNLASGIPARFSGSGSGTDFTLTISSLEPEDFAVYYCQQASSIPRMFTFGQ |
| | GTKLEIK (SEQ ID NO: 372) |
| 10-19 | Heavy |
| | EVKLQESGPGLVAPSQSLSVTCTVSGVSLPDYGVSWIRQPPRKGLEWLG |
| | VIWGSETTYYNSALKSRLTIIKDNSKSQVFLKMNSLQTDDTAIYYCAKH |
| | YYYGGSYAMDYWGQGTSVTVSS (SEQ ID NO: 369) |
| | Light |
| | DIQMTQTTSSLSASLGDRVTISCRASQDISKYLNWYQQKPDGTVKLLIYH |
| | TSRLHSGVPSRFSGSGSGTDYSLTISNLEQEDIATYFCQQGNTLPYTFGGG |
| | TKLEIT (SEQ ID NO: 370) |
| 10-20 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-21 | Heavy |
| | QVTLRESGPALVKPTQTLTLTCTFSGFSLSTSGMGVGWIRQPPGKALEW |
| | LAHIWWDDDKRYNPALKSRLTISKDTSKNQVFLTMTNMDPVDTATYY |
| | CARMELWSYYFDYWGQGTTVTVSS (SEQ ID NO: 375) |
| | Light |
| | ENVLTQSPATLSVTPGEKATITCRASQSVSYMHWYQQKPGQAPRLLIYD |
| | ASNRASGVPSRFSGSGSGTDHTLTISSLEAEDAATYYCFQGSVYPFTFG |
| | QGTKLEIK (SEQ ID NO: 376) |
| 10-22 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-23 | Heavy |
| | EVQLVQSGAEVKKPGESLKISCKGSGYSFSSSWIGWVRQMPGKGLEWM |
| | GIIYPDDSDTRYSPSFQGQVTISADKSIRTAYLQWSSLKASDTAMYYCAR |
| | HVTMIWGVIIDFWGQGTLVTVSS (SEQ ID NO: 379) |
| | Light |
| | AIQLTQSPSSLSASVGDRVTITCRASQGISSALAWYQQKPGKAPKLLIYD |
| | ASSLESGVPSRFSGSGSGTDFTLTISSLQPEDFATYYCQQFNSYPYTFGQG |
| | TKLEIK (SEQ ID NO: 380) |
| 10-24 | Heavy |
| | QVQLVQSGAEVKKPGSSVKVSCKASGHTISSYAYSWVRQAPGQGLEW |
| | MGDIIPAYGSPNYAQKFQGRVTITADESTSTAYMELSSLRSEDTAVYYC |
| | AREDFGKNYAMDVWGQGTLVTVSS (SEQ ID NO: 381) |
| | Light |
| | EIVLTQSPGTLSLSPGERATLSCRASQHVSSHYLAWYQQKPGQAPRLLIY |
| | GASSRATGIPDRFSGSGSGTDFTLTISRLEPEDFAVYYCQHYGQSQFTFG |
| | QGTKVEIK (SEQ ID NO: 382) |
| 10-25 | Heavy |
| | QVQLVQSGAEVKKPGSSVKVSCKASGHTISSYAYSWVRQAPGQGLEW |
| | MGDIIPAYGSPNYAQKFQGRVTITADESTSTAYMELSSLRSEDTAVYYC |
| | AREDFGKNYAMDVWGQGTLVTVSS (SEQ ID NO: 381) |
| | Light |
| | EIVLTQSPGTLSLSPGERATLSCRASQHVSSHYLAWYQQKPGQAPRLLIY |
| | GASSRATGIPDRESGSGSGTDFTLTISRLEPEDFAVYYCQHYGQSQFTFG |
| | QGTKVEIK (SEQ ID NO: 382) |
| 10-26 | Heavy |
| | QVQLVQSGAEVKKPGASVKVSCKASGYDFTDYIMHWVRQAPGQCLEW |
| | MGYINPYNDGSKYTDKFQERVTMTSDTSISTAYMELSRLRSDDTAVYY |
| | CARGTYYYGPELFDYWGQGTTVTVSS (SEQ ID NO: 383) |
| | Light |
| | DIVMTQTPLSLSVTPGQPASISCKSSQSLETTTGTTYLNWYLQKPGQSP |
| | QLLIYRASKRFSGVPDRFSGSGSGTDFTLKISRVEAEDVGVYYCLQLLE |
| | DPYTFGCGTKLEIK (SEQ ID NO: 384) |
| 10-27 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-28 | Heavy |
| | QVQLVQSGAEVKKPGSSVKVSCKASGYTFTDYVIHWVRQAPGQGLEW |
| | MGYFNPYNDGTEYNEKFKARVTITADKSTSTAYMELSSLRSEDTAVYY |
| | CARGPYYYGSSPFDYWGQGTTVTVSS (SEQ ID NO: 387) |
| | Light |
| | DIVMTQTPLSLPVTPGEPASISCRSSQSLENSNHNTYINWYLQKPGQSPQL |
| | LIYRVSKRFSGVPDRFSGSGSGTDFTLKISRVEAEDVGVYYCHQVTHVPY |
| | TFGQGTKLEIK (SEQ ID NO: 388) |
| 10-29 | Heavy |
| | QVQLVQSGAEVKKPGSSVKVSCKASGYTFTDYVIHWVRQAPGQGLEW |
| | MGYFNPYNDGTEYNEKFKARVTITADKSTSTAYMELSSLRSEDTAVYY |
| | CARGPYYYGSSPFDYWGQGTTVTVSS (SEQ ID NO: 387) |
| | Light |
| | DIVMTQTPLSLPVTPGEPASISCRSSQSLENSNHNTYINWYLQKPGQSPQL |
| | LIYRVSKRFSGVPDRFSGSGSGTDFTLKISRVEAEDVGVYYCHQVTHVPY |
| | TFGQGTKLEIK (SEQ ID NO: 388) |
| 10-30 | Heavy |
| | QVQLVQSGAEVKKPGSSVKVSCKASGYTFTDYVIHWVRQAPGQGLEW |
| | MGYFNPYNDGTEYNEKFKARVTITADKSTSTAYMELSSLRSEDTAVYY |
| | CARGPYYYGSSPFDYWGQGTTVTVSS (SEQ ID NO: 387) |
| | Light |
| | DIVMTQTPLSLPVTPGEPASISCRSSQSLENSNHNTYINWYLQKPGQSPQL |
| | LIYRVSKRFSGVPDRFSGSGSGTDFTLKISRVEAEDVGVYYCHQVTHVPY |
| | TFGQGTKLEIK (SEQ ID NO: 388) |
| 10-31 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-32 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-33 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-34 | Heavy |
| | QVQLVQSGAEVKKPGSSVKVSCKASGYTFTDYVIHWVRQAPGQGLEW |
| | MGYFNPYNDGTEYNEKFKARVTITADKSTSTAYMELSSLRSEDTAVYY |
| | CARGPYYYGSSPFDYWGQGTTVTVSS (SEQ ID NO: 387) |
| | Light |
| | DIVMTQTPLSLPVTPGEPASISCRSSQSLENSNHNTYINWYLQKPGQSPQL |
| | LIYRVSKRFSGVPDRFSGSGSGTDFTLKISRVEAEDVGVYYCHQVTHVPY |
| | TFGQGTKLEIK (SEQ ID NO: 388) |
| 10-35 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-36 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-37 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-38 | Heavy |
| | QVQLVQSGAEVKKPGSSVKVSCKASGYTFTDYVIHWVRQAPGQGLEW |
| | MGYFNPYNDGTEYNEKFKARVTITADKSTSTAYMELSSLRSEDTAVYY |
| | CARGPYYYGSSPFDYWGQGTTVTVSS (SEQ ID NO: 387) |
| | Light |
| | DIVMTQTPLSLPVTPGEPASISCRSSQSLENSNHNTYINWYLQKPGQSPQL |
| | LIYRVSKRFSGVPDRFSGSGSGTDFTLKISRVEAEDVGVYYCHQVTHVPY |
| | TFGQGTKLEIK (SEQ ID NO: 388) |
| 10-39 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-40 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-41 | Heavy |
| | QVQLQESGPGLVKPSQTLSLTCTVSGGSISTSTMGVGWIRQHPGKGLEW |
| | IGFIWWDDDKRYNPNLKSRVTMSVDTSKNQFSLKLSSVTAADTAVYY |
| | CARMELWSYYFDYWGQGTLVTVSS (SEQ ID NO: 385) |
| | Light |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIK (SEQ ID NO: 386) |
| 10-42 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-43 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-44 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-45 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-46 | Heavy |
| | EVQLVESGGGLVKPGGSLKLSCAASGYTFTSYVMHWVRQAPGKGLEWI |
| | GYINPYNDGTKYNEKFQGRVTISSDKSISTAYMELSSLRSEDTAMYYCA |
| | RGTYYYGTRVFDYWGQGTLVTVSS (SEQ ID NO: 373) |
| | Light |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIK (SEQ ID NO: 374) |
| 10-47 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-48 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-49 | Heavy |
| | QVQLQESGPGLVKPSQTLSLTCTVSGGSISTSGMGVGWIRQHPGKGLEW |
| | IGHIWWDDDKRYNPALKSRVTISVDTSKNQFSLKLSSVTAADTAVYYCA |
| | RMELWSYYFDYWGQGTLVTVSS (SEQ ID NO: 377) |
| | Light |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIK (SEQ ID NO: 378) |
| 10-50 | scFv |
| | DIQMTQTTSSLSASLGDRVTISCRASQDISKYLNWYQQKPDGTVKLLIYH |
| | TSRLHSGVPSRFSGSGSGTDYSLTISNLEQEDIATYFCQQGNTLPYTFGGG |
| | TKLEITGGGGSGGGGSGGGGSEVKLQESGPGLVAPSQSLSVTCTVSGVS |
| | LPDYGVSWIRQPPRKGLEWLGVIWGSETTYYNSALKSRLTIIKDNSKSQV |
| | FLKMNSLQTDDTAIYYCAKHYYYGGSYAMDYWGQGTSVTVSS (SEQ |
| | ID NO: 389) |
| 10-51 | scFv |
| | EIVLTQSPATLSLSPGERATLSCSASSSISSNYLHWYQQKPGQAPRFLIYR |
| | TSNLASGIPARFSGSGSGTDFTLTISSLEPEDFAVYYCQQASSIPRMFTFGQ |
| | GTKLEIKGSTSGSGKPGSGEGSTKGEVQLQQSGAEVKKPGASVKVSCKA |
| | SGYTFTNYWMHWVRQRPGQGLEWMGEIDPSDNYANYNQEFQGRVTIT |
| | VDKSASTAYMELSSLRSEDTAVYYCARHDGYFGALDYWGQGTTVTVSS |
| | (SEQ ID NO: 390) |
| 10-53 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-54 | scFv |
| | QVTLRESGPALVKPTQTLTLTCTFSGFSLSTSGMGVGWIRQPPGKALEW |
| | LAHIWWDDDKRYNPALKSRLTISKDTSKNQVFLTMTNMDPVDTATYY |
| | CARMELWSYYFDYWGQGTTVTVSSGGGGSGGGGSGGGGSENVLTQSP |
| | ATLSVTPGEKATITCRASQSVSYMHWYQQKPGQAPRLLIYDASNRASG |
| | VPSRFSGSGSGTDHTLTISSLEAEDAATYYCFQGSVYPFTFGQGTKLEIK |
| | (SEQ ID NO: 392) |
| 10-55 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-56 | scFv |
| | EVQLVQSGAEVKKPGESLKISCKGSGYSFSSSWIGWVRQMPGKGLEWM |
| | GIIYPDDSDTRYSPSFQGQVTISADKSIRTAYLQWSSLKASDTAMYYCAR |
| | HVTMIWGVIIDFWGQGTLVTVSSGGGGSGGGGSGGGGSAIQLTQSPSSL |
| | SASVGDRVTITCRASQGISSALAWYQQKPGKAPKLLIYDASSLESGVPSR |
| | FSGSGSGTDFTLTISSLQPEDFATYYCQQFNSYPYTFGQGTKLEIK (SEQ |
| | ID NO: 394) |
| 10-57 | scFv |
| | QVQLVQSGAEVKKPGSSVKVSCKASGHTISSYAYSWVRQAPGQGLEW |
| | MGDIIPAYGSPNYAQKFQGRVTITADESTSTAYMELSSLRSEDTAVYYC |
| | AREDFGKNYAMDVWGQGTLVTVSSGGGGSGGGGSGGGGSEIVLTQSP |
| | GTLSLSPGERATLSCRASQHVSSHYLAWYQQKPGQAPRLLIYGASSRAT |
| | GIPDRFSGSGSGTDFTLTISRLEPEDFAVYYCQHYGQSQFTFGQGTKVEI |
| | K (SEQ ID NO: 395) |
| 10-58 | scFv |
| | EIVLTQSPGTLSLSPGERATLSCRASQHVSSHYLAWYQQKPGQAPRLLIY |
| | GASSRATGIPDRFSGSGSGTDFTLTISRLEPEDFAVYYCQHYGQSQFTFG |
| | QGTKVEIKGGGGSGGGGSGGGGSQVQLVQSGAEVKKPGSSVKVSCKAS |
| | GHTISSYAYSWVRQAPGQGLEWMGDIIPAYGSPNYAQKFQGRVTITAD |
| | ESTSTAYMELSSLRSEDTAVYYCAREDFGKNYAMDVWGQGTLVTVSS |
| | (SEQ ID NO: 396) |
| 10-59 | scFv |
| | DIVMTQTPLSLSVTPGQPASISCKSSQSLETTTGTTYLNWYLQKPGQSP |
| | QLLIYRASKRFSGVPDRFSGSGSGTDFTLKISRVEAEDVGVYYCLQLLE |
| | DPYTFGCGTKLEIKGGGGSGGGGSGGGGSQVQLVQSGAEVKKPGASVK |
| | VSCKASGYDFTDYIMHWVRQAPGQCLEWMGYINPYNDGSKYTDKFQ |
| | ERVTMTSDTSISTAYMELSRLRSDDTAVYYCARGTYYYGPELFDYWG |
| | QGTTVTVSS (SEQ ID NO: 397) |
| 10-60 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-64 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-65 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-66 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-68 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-69 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-70 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-72 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-73 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-74 | scFv |
| | EIVLTQSPATLSLSPGERATLSCSASSSVGYMHWYQQKPGQAPRLLIYDT |
| | SKLASGIPARFSGSGSGTDFTLTISSLEPEDFAVYYCFQGSVYPFTFGQGT |
| | KLEIKGGGGSGGGGSGGGGSQVQLQESGPGLVKPSQTLSLTCTVSGGSIS |
| | TSTMGVGWIRQHPGKGLEWIGFIWWDDDKRYNPNLKSRVTMSVDTS |
| | KNQFSLKLSSVTAADTAVYYCARMELWSYYFDYWGQGTLVTVSS |
| | (SEQ ID NO: 398) |
| 10-75 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSGGGGSGGGGSEVQLVESGG |
| | GLVKPGGSLKLSCAASGYTFTSYVMHWVRQAPGKGLEWIGYINPYND |
| | GTKYNEKFQGRVTISSDKSISTAYMELSSLRSEDTAMYYCARGTYYYGT |
| | RVFDYWGQGTLVTVSS (SEQ ID NO: 399) |
| 10-76 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGSGGGSGGGGSGGGGSQVQLQESGPGLVKPSQTLSLT |
| | CTVSGGSISTSGMGVGWIRQHPGKGLEWIGHIWWDDDKRYNPALKSRV |
| | TISVDTSKNQFSLKLSSVTAADTAVYYCARMELWSYYFDYWGQGTLVT |
| | VSS (SEQ ID NO: 400) |
| 10-77 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-78 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-79 | scFv |
| | DIVMTQSPATLSLSPGERATLSCRSSKSLQNVNGNTYLYWFQQKPGQSP |
| | QLLIYRMSNLNSGVPDRFSGSGSGTEFTLTISSLEPEDFAVYYCMQHLE |
| | YPITFGAGTKLEIKGGGGSGGGGSGGGGSEVQLVESGGGLVKPGGSLKL |
| | SCAASGYTFTSYVMHWVRQAPGKGLEWIGYINPYNDGTKYNEKFQGR |
| | VTISSDKSISTAYMELSSLRSEDTAMYYCARGTYYYGTRVFDYWGQGT |
| | LVTVSS (SEQ ID NO: 391) |
| 10-80 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-81 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-82 | scFv |
| | EIVLTQSPATLSLSPGERATLSCSASSSVSYMHWYQQKPGQAPRLLIYDT |
| | SKLASGIPARFSGSGSGTDFTLTISSLEPEDVAVYYCFQGSVYPFTFGQGT |
| | KLEIKGGGGSGGGGSGGGGSQVQLQESGPGLVKPSQTLSLTCTVSGGSIS |
| | TSGMGVGWIRQHPGKGLEWIGHIWWDDDKRYNPALKSRVTISVDTSKN |
| | QFSLKLSSVTAADTAVYYCARMELWSYYFDYWGQGTLVTVSS (SEQ ID |
| | NO: 393) |
| 10-83 | CAR |
| | MALPVTALLLPLALLLHAARPDIQMTQTTSSLSASLGDRVTISCRASQDIS |
| | KYLNWYQQKPDGTVKLLIYHTSRLHSGVPSRFSGSGSGTDYSLTISNLEQ |
| | EDIATYFCQQGNTLPYTFGGGTKLEITGGGGSGGGGSGGGGSEVKLQES |
| | GPGLVAPSQSLSVTCTVSGVSLPDYGVSWIRQPPRKGLEWLGVIWGSET |
| | TYYNSALKSRLTIIKDNSKSQVFLKMNSLQTDDTAIYYCAKHYYYGGSY |
| | AMDYWGQGTSVTVSSIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNEL |
| | NLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID |
| | NO: 280) |
| 10-84 | CAR |
| | MLLLVTSLLLCELPHPAFLLIPEIVLTQSPATLSLSPGERATLSCSASSSISS |
| | NYLHWYQQKPGQAPRFLIYRTSNLASGIPARFSGSGSGTDFTLTISSLEPE |
| | DFAVYYCQQASSIPRMFTFGQGTKLEIKGSTSGSGKPGSGEGSTKGEVQL |
| | QQSGAEVKKPGASVKVSCKASGYTFTNYWMHWVRQRPGQGLEWMGE |
| | IDPSDNYANYNQEFQGRVTITVDKSASTAYMELSSLRSEDTAVYYCARH |
| | DGYFGALDYWGQGTTVTVSSAAAIEVMYPPPYLDNEKSNGTIIHVKGK |
| | HLCPSPLFPGPSKPFWVLVVVGGVLACYSLLVTVAFIIFWVRSKRSRLLH |
| | SDYMNMTPRRPGPTRKHYQPYAPPRDFAAYRSRVKFSRSADAPAYQQG |
| | QNQLYNELNLGRREEYDVLDKRRGRDPEMGGKPRRKNPQEGLYNELQ |
| | KDKMAEAYSEIGMKGERRRGKGHDGLYQGLSTATKDTYDALHMQALP |
| | PR (SEQ ID NO: 287) |
| 10-86 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSKRSR |
| | LLHSDYMNMTPRRPGPTRKHYQPYAPPRDFAAYRSRVKFSRSADAPAY |
| | QQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPR (SEQ ID NO: 288) |
| 10-87 | CAR |
| | MALPVTALLLPLALLLHAARPQVTLRESGPALVKPTQTLTLTCTFSGFSL |
| | STSGMGVGWIRQPPGKALEWLAHIWWDDDKRYNPALKSRLTISKDTS |
| | KNQVFLTMTNMDPVDTATYYCARMELWSYYFDYWGQGTTVTVSSGG |
| | GGSGGGGSGGGGSENVLTQSPATLSVTPGEKATITCRASQSVSYMHWY |
| | QQKPGQAPRLLIYDASNRASGVPSRFSGSGSGTDHTLTISSLEAEDAATY |
| | YCFQGSVYPFTFGQGTKLEIKIEVMYPPPYLDNEKSNGTIIHVKGKHLCP |
| | SPLFPGPSKPFWVLVVVGGVLACYSLLVTVAFIIFWVRSKRSRLLHSDY |
| | MNMTPRRPGPTRKHYQPYAPPRDFAAYRSRVKFSRSADAPAYQQGQNQ |
| | LYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| | (SEQ ID NO: 289) |
| 10-88 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNEL |
| | NLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID |
| | NO: 290) |
| 10-89 | CAR |
| | MALPVTALLLPLALLLHAARPEVQLVQSGAEVKKPGESLKISCKGSGYS |
| | FSSSWIGWVRQMPGKGLEWMGIIYPDDSDTRYSPSFQGQVTISADKSIRT |
| | AYLQWSSLKASDTAMYYCARHVTMIWGVIIDFWGQGTLVTVSSGGGGS |
| | GGGGSGGGGSAIQLTQSPSSLSASVGDRVTITCRASQGISSALAWYQQKP |
| | GKAPKLLIYDASSLESGVPSRFSGSGSGTDFTLTISSLQPEDFATYYCQQF |
| | NSYPYTFGQGTKLEIKIEVMYPPPYLDNEKSNGTIIHVKGKHLCPSPLFPG |
| | PSKPFWVLVVVGGVLACYSLLVTVAFIIFWVRSKRSRLLHSDYMNMTPR |
| | RPGPTRKHYQPYAPPRDFAAYRSRVKFSRSADAPAYQQGQNQLYNELN |
| | LGRREEYDVLDKRRGRDPEMGGKPRRKNPQEGLYNELQKDKMAEAYS |
| | EIGMKGERRRGKGHDGLYQGLSTATKDTYDALHMQALPPR (SEQ ID |
| | NO: 291) |
| 10-90 | CAR |
| | MALPVTALLLPLALLLHAARPQVQLVQSGAEVKKPGSSVKVSCKASGH |
| | TISSYAYSWVRQAPGQGLEWMGDIIPAYGSPNYAQKFQGRVTITADEST |
| | STAYMELSSLRSEDTAVYYCAREDFGKNYAMDVWGQGTLVTVSSGGG |
| | GSGGGGSGGGGSEIVLTQSPGTLSLSPGERATLSCRASQHVSSHYLAWY |
| | QQKPGQAPRLLIYGASSRATGIPDRFSGSGSGTDFTLTISRLEPEDFAVYY |
| | CQHYGQSQFTFGQGTKVEIKIEVMYPPPYLDNEKSNGTIIHVKGKHLCP |
| | SPLFPGPSKPFWVLVVVGGVLACYSLLVTVAFIIFWVRSKRSRLLHSDY |
| | MNMTPRRPGPTRKHYQPYAPPRDFAAYRSRVKFSRSADAPAYQQGQNQ |
| | LYNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| | (SEQ ID NO: 292) |
| 10-91 | CAR |
| | MALPVTALLLPLALLLHAARPEIVLTQSPGTLSLSPGERATLSCRASQHV |
| | SSHYLAWYQQKPGQAPRLLIYGASSRATGIPDRFSGSGSGTDFTLTISRL |
| | EPEDFAVYYCQHYGQSQFTFGQGTKVEIKGGGGSGGGGSGGGGSQVQ |
| | LVQSGAEVKKPGSSVKVSCKASGHTISSYAYSWVRQAPGQGLEWMGD |
| | IIPAYGSPNYAQKFQGRVTITADESTSTAYMELSSLRSEDTAVYYCARE |
| | DFGKNYAMDVWGQGTLVTVSSIEVMYPPPYLDNEKSNGTIIHVKGKHL |
| | CPSPLFPGPSKPFWVLVVVGGVLACYSLLVTVAFIIFWVRSKRSRLLHSD |
| | YMNMTPRRPGPTRKHYQPYAPPRDFAAYRSRVKFSRSADAPAYQQGQN |
| | QLYNELNLGRREEYDVLDKRRGRDPEMGGKPRRKNPQEGLYNELQKD |
| | KMAEAYSEIGMKGERRRGKGHDGLYQGLSTATKDTYDALHMQALPPR |
| | (SEQ ID NO: 293) |
| 10-92 | CAR |
| | MALPVTALLLPLALLLHAARPDIVMTQTPLSLSVTPGQPASISCKSSQSL |
| | ETTTGTTYLNWYLQKPGQSPQLLIYRASKRFSGVPDRFSGSGSGTDFTL |
| | KISRVEAEDVGVYYCLQLLEDPYTFGCGTKLEIKGGGGSGGGGSGGGG |
| | SQVQLVQSGAEVKKPGASVKVSCKASGYDFTDYIMHWVRQAPGQCLE |
| | WMGYINPYNDGSKYTDKFQERVTMTSDTSISTAYMELSRLRSDDTAV |
| | YYCARGTYYYGPELFDYWGQGTTVTVSSIEVMYPPPYLDNEKSNGTIIH |
| | VKGKHLCPSPLFPGPSKPFWVLVVVGGVLACYSLLVTVAFIIFWVRSKRS |
| | RLLHSDYMNMTPRRPGPTRKHYQPYAPPRDFAAYRSRVKFSRSADAPA |
| | YQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHM |
| | QALPPR (SEQ ID NO: 294) |
| 10-93 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSKRSRLLHSDYMN |
| | MTPRRPGPTRKHYQPYAPPRDFAAYRSRVKFSRSADAPAYQQGQNQLY |
| | NELNLGRREEYDVLDKRRGRDPEMGGKPRRKNPQEGLYNELQKDKMA |
| | EAYSEIGMKGERRRGKGHDGLYQGLSTATKDTYDALHMQALPPR (SEQ |
| | ID NO: 295) |
| 10-97 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSKRSRLLHSDYMN |
| | MTPRRPGPTRKHYQPYAPPRDFAAYRSRVKFSRSADAPAYQQGQNQLY |
| | NELNLGRREEYDVLDKRRGRDPEMGGKPRRKNPQEGLYNELQKDKMA |
| | EAYSEIGMKGERRRGKGHDGLYQGLSTATKDTYDALHMQALPPR (SEQ |
| | ID NO: 295) |
| 10-98 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSKRSRLLHSDYMN |
| | MTPRRPGPTRKHYQPYAPPRDFAAYRSRVKFSRSADAPAYQQGQNQLY |
| | NELNLGRREEYDVLDKRRGRDPEMGGKPRRKNPQEGLYNELQKDKMA |
| | EAYSEIGMKGERRRGKGHDGLYQGLSTATKDTYDALHMQALPPR (SEQ |
| | ID NO: 295) |
| 10-99 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSKRSRLLHSDYMN |
| | MTPRRPGPTRKHYQPYAPPRDFAAYRSRVKFSRSADAPAYQQGQNQLY |
| | NELNLGRREEYDVLDKRRGRDPEMGGKPRRKNPQEGLYNELQKDKMA |
| | EAYSEIGMKGERRRGKGHDGLYQGLSTATKDTYDALHMQALPPR (SEQ |
| | ID NO: 295) |
| 10-101 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSRRSR |
| | LLHSDYMNMTPRRPGPTRRHYQPYAPPRDFAAYRSRVRFSRSADAPAY |
| | QQGQNQLYNELNLGRREEYDVLDRRRGRDPEMGGRPRRRNPQEGLYN |
| | ELQRDRMAEAYSEIGMRGERRRGRGHDGLYQGLSTATRDTYDALHMQ |
| | ALPPR (SEQ ID NO: 296) |
| 10-102 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSRRSRLLHSDYMNMTP |
| | RRPGPTRRHYQPYAPPRDFAAYRSRVRFSRSADAPAYQQGQNQLYNEL |
| | NLGRREEYDVLDRRRGRDPEMGGRPRRRNPQEGLYNELQRDRMAEAY |
| | SEIGMRGERRRGRGHDGLYQGLSTATRDTYDALHMQALPPR (SEQ ID |
| | NO: 297) |
| 10-103 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSRRSRLLHSDYMN |
| | MTPRRPGPTRRHYQPYAPPRDFAAYRSRVRFSRSADAPAYQQGQNQLY |
| | NELNLGRREEYDVLDRRRGRDPEMGGRPRRRNPQEGLYNELQRDRMA |
| | EAYSEIGMRGERRRGRGHDGLYQGLSTATRDTYDALHMQALPPR (SEQ |
| | ID NO: 298) |
| 10-105 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSRRSR |
| | LLHSDYMNMTPRRPGPTRRHYQPYAPPRDFAAYRSRVRFSRSADAPAY |
| | QQGQNQLYNELNLGRREEYDVLDRRRGRDPEMGGRPRRRNPQEGLYN |
| | ELQRDRMAEAYSEIGMRGERRRGRGHDGLYQGLSTATRDTYDALHMQ |
| | ALPPR (SEQ ID NO: 296) |
| 10-106 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSRRSRLLHSDYMNMTP |
| | RRPGPTRRHYQPYAPPRDFAAYRSRVRFSRSADAPAYQQGQNQLYNEL |
| | NLGRREEYDVLDRRRGRDPEMGGRPRRRNPQEGLYNELQRDRMAEAY |
| | SEIGMRGERRRGRGHDGLYQGLSTATRDTYDALHMQALPPR (SEQ ID |
| | NO: 297) |
| 10-107 | CAR |
| | MALPVTALLLPLALLLHAARPEIVLTQSPATLSLSPGERATLSCSASSSVG |
| | YMHWYQQKPGQAPRLLIYDTSKLASGIPARFSGSGSGTDFTLTISSLEPE |
| | DFAVYYCFQGSVYPFTFGQGTKLEIKGGGGSGGGGSGGGGSQVQLQES |
| | GPGLVKPSQTLSLTCTVSGGSISTSTMGVGWIRQHPGKGLEWIGFIWW |
| | DDDKRYNPNLKSRVTMSVDTSKNQFSLKLSSVTAADTAVYYCARMEL |
| | WSYYFDYWGQGTLVTVSSIEVMYPPPYLDNEKSNGTIIHVKGKHLCPSP |
| | LFPGPSKPFWVLVVVGGVLACYSLLVTVAFIIFWVRSRRSRLLHSDYMN |
| | MTPRRPGPTRRHYQPYAPPRDFAAYRSRVRFSRSADAPAYQQGQNQLY |
| | NELNLGRREEYDVLDRRRGRDPEMGGRPRRRNPQEGLYNELQRDRMA |
| | EAYSEIGMRGERRRGRGHDGLYQGLSTATRDTYDALHMQALPPR (SEQ |
| | ID NO: 298) |
| 10-108 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SGGGGSGGGGSEVQLVESGGGLVKPGGSLKLSCAASGYTFTSYVMHW |
| | VRQAPGKGLEWIGYINPYNDGTKYNEKFQGRVTISSDKSISTAYMELSSL |
| | RSEDTAMYYCARGTYYYGTRVFDYWGQGTLVTVSSIEVMYPPPYLDN |
| | EKSNGTIIHVKGKHLCPSPLFPGPSKPFWVLVVVGGVLACYSLLVTVAFII |
| | FWVRSKRSRLLHSDYMNMTPRRPGPTRKHYQPYAPPRDFAAYRSRVKF |
| | SRSADAPAYQQGQNQLYNELNLGRREEYDVLDKRRGRDPEMGGKPRR |
| | KNPQEGLYNELQKDKMAEAYSEIGMKGERRRGKGHDGLYQGLSTATK |
| | DTYDALHMQALPPR (SEQ ID NO: 299) |
| 10-109 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGSGGGSGGGGSGGGG |
| | SQVQLQESGPGLVKPSQTLSLTCTVSGGSISTSGMGVGWIRQHPGKGLE |
| | WIGHIWWDDDKRYNPALKSRVTISVDTSKNQFSLKLSSVTAADTAVYY |
| | CARMELWSYYFDYWGQGTLVTVSSIEVMYPPPYLDNEKSNGTIIHVKG |
| | KHLCPSPLFPGPSKPFWVLVVVGGVLACYSLLVTVAFIIFWVRSKRSRLL |
| | HSDYMNMTPRRPGPTRKHYQPYAPPRDFAAYRSRVKFSRSADAPAYQQ |
| | GQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQAL |
| | PPR (SEQ ID NO: 300) |
| 10-110 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSKRSR |
| | LLHSDYMNMTPRRPGPTRKHYQPYAPPRDFAAYRSRVKFSRSADAPAY |
| | QQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPR (SEQ ID NO: 288) |
| 10-111 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSKRSR |
| | LLHSDYMNMTPRRPGPTRKHYQPYAPPRDFAAYRSRVKFSRSADAPAY |
| | QQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPR (SEQ ID NO: 288) |
| 10-112 | CAR |
| | MALPVTALLLPLALLLHAARPDIVMTQSPATLSLSPGERATLSCRSSKSL |
| | QNVNGNTYLYWFQQKPGQSPQLLIYRMSNLNSGVPDRFSGSGSGTEFT |
| | LTISSLEPEDFAVYYCMQHLEYPITFGAGTKLEIKGGGGSGGGGSGGGG |
| | SEVQLVESGGGLVKPGGSLKLSCAASGYTFTSYVMHWVRQAPGKGLE |
| | WIGYINPYNDGTKYNEKFQGRVTISSDKSISTAYMELSSLRSEDTAMYY |
| | CARGTYYYGTRVFDYWGQGTLVTVSSIEVMYPPPYLDNEKSNGTIIHV |
| | KGKHLCPSPLFPGPSKPFWVLVVVGGVLACYSLLVTVAFIIFWVRSKRSR |
| | LLHSDYMNMTPRRPGPTRKHYQPYAPPRDFAAYRSRVKFSRSADAPAY |
| | QQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPR (SEQ ID NO: 288) |
| 10-113 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNEL |
| | NLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID |
| | NO: 290) |
| 10-114 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNEL |
| | NLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID |
| | NO: 290) |
| 10-115 | CAR |
| | MALPVTALLLPLALLLHAARPEIVLTQSPATLSLSPGERATLSCSASSSVS |
| | YMHWYQQKPGQAPRLLIYDTSKLASGIPARFSGSGSGTDFTLTISSLEPE |
| | DVAVYYCFQGSVYPFTFGQGTKLEIKGGGGSGGGGSGGGGSQVQLQES |
| | GPGLVKPSQTLSLTCTVSGGSISTSGMGVGWIRQHPGKGLEWIGHIWWD |
| | DDKRYNPALKSRVTISVDTSKNQFSLKLSSVTAADTAVYYCARMELWS |
| | YYFDYWGQGTLVTVSSIEVMYPPPYLDNEKSNGTIIHVKGKHLCPSPLFP |
| | GPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNEL |
| | NLGRREEYDVLDKRRGRDPEMGGKPRRKNPQEGLYNELQKDKMAEAY |
| | SEIGMKGERRRGKGHDGLYQGLSTATKDTYDALHMQALPPR (SEQ ID |
| | NO: 290) |

In some embodiments, the circular RNA comprises more than 1 expression sequence, e.g., 2, 3, 4, or 5 expression sequences. In some embodiments, the circular RNA is a bicistronic RNA. In some embodiments, the bicistronic RNA is codon optimized. Exemplary bicistronic circular RNA are described in WO2021/189059A2, which is incorporated by reference herein in its entirety.

In some embodiments, the scFv, heavy variable domain, light variable domain, heavy CDR sequences, and/or light CDR sequences of the proteins listed in the tables herein may be used.

In some embodiments, the therapeutic protein is selected from, e.g., a CD19-targted chimeric antigen receptor (CAR), a BCMA-targeted CAR, MAGE-A4 T-cell receptor (TCR), NY-ESO TCR, erythropoietin (EPO), phenylalanine hydroxylase (PAH), carbamoyl phosphate synthetase I (CPS1), Cas9, ADAMTS13, FOXP3, IL-10, or IL-2. Exemplary sequences are provided herein.

In some embodiments, the expression sequence encodes a therapeutic protein. In some embodiments, the expression sequence encodes, e.g., a cytokine, e.g., IL-12p70, IL-15, IL-2, IL-18, IL-21, IFN-α, IFN-β, IL-10, TGF-beta, IL-4, or IL-35, or a functional fragment thereof. In some embodiments, the expression sequence encodes, e.g., an immune checkpoint inhibitor. In some embodiments, the expression sequence encodes, e.g., an agonist (e.g., a TNFR family member such as CD137L, OX40L, ICOSL, LIGHT, or CD70). In some embodiments, the expression sequence encodes, e.g., a chimeric antigen receptor. In some embodiments, the expression sequence encodes, e.g., an inhibitory receptor agonist (e.g., PDL1, PDL2, Galectin-9, VISTA, B7H4, or MHCII) or inhibitory receptor (e.g., PD1, CTLA4, TIGIT, LAG3, or TIM3). In some embodiments, the expression sequence encodes, e.g., an inhibitory receptor antagonist. In some embodiments, the expression sequence encodes, e.g., one or more TCR chains (alpha and beta chains or gamma and delta chains). In some embodiments, the expression sequence encodes, e.g., a secreted T cell or immune cell engager (e.g., a bispecific antibody such as BiTE, targeting, e.g., CD3, CD137, or CD28 and a tumor-expressed protein e.g., CD19 etc.). In some embodiments, the expression sequence encodes, e.g., a transcription factor (e.g., FOXP3, HELIOS, TOX1, or TOX2). In some embodiments, the expression sequence encodes an immunosuppressive enzyme (e.g., IDO or CD39/CD73). In some embodiments, the expression sequence encodes, e.g., a GvHD (e.g., anti-HLA-A2 CAR-Tregs).

In some embodiments, a provided polynucleotide encodes a protein that is made up of subunits that are encoded by more than one gene. For example, the protein may be a heterodimer, wherein each chain or subunit of the protein is encoded by a separate gene. It is possible that more than one provided polynucleotides (e.g., circular RNA polynucleotides) are delivered in the transfer vehicle and each polynucleotide encodes a separate subunit of the protein. In certain embodiments, polynucleotides encoding the individual subunits may be administered in separate transfer vehicles. Alternatively, a single polynucleotide (e.g., circular RNA polynucleotide) may be engineered to encode more than one subunit.

ii. Hinge/Spacer Domain

In some embodiments, a CAR of the instant disclosure comprises a hinge or spacer domain. In some embodiments, the hinge/spacer domain may comprise a truncated hinge/spacer domain (THD) the THD domain is a truncated version of a complete hinge/spacer domain (“CHD”). In some embodiments, an extracellular domain is from or derived from (e.g., comprises all or a fragment of) ErbB2, glycophorin A (GpA), CD2, CD3 delta, CD3 epsilon, CD3 gamma, CD4, CD7, CD8a, CD8[T CDl la (IT GAL), CDl lb (IT GAM), CDl lc (ITGAX), CDl ld (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (0X40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CDl la/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge or spacer domain may be derived either from a natural or from a synthetic source.

In some embodiments, a hinge or spacer domain is positioned between an antigen binding molecule (e.g., an scFv or VHH) and a transmembrane domain. In this orientation, the hinge/spacer domain provides distance between the antigen binding molecule and the surface of a cell membrane on which the CAR is expressed. In some embodiments, a hinge or spacer domain is from or derived from an immunoglobulin. In some embodiments, a hinge or spacer domain is selected from the hinge/spacer regions of IgG1, IgG2, IgG3, IgG4, IgA, IgD, IgE, IgM, or a fragment thereof. In some embodiments, a hinge or spacer domain comprises, is from, or is derived from the hinge/spacer region of CD8 alpha. In some embodiments, a hinge or spacer domain comprises, is from, or is derived from the hinge/spacer region of CD28. In some embodiments, a hinge or spacer domain comprises a fragment of the hinge/spacer region of CD8 alpha or a fragment of the hinge/spacer region of CD28, wherein the fragment is anything less than the whole hinge/spacer region. In some embodiments, the fragment of the CD8 alpha hinge/spacer region or the fragment of the CD28 hinge/spacer region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge/spacer region, or of the CD28 hinge/spacer region.

iii. Transmembrane Domain

The CAR of the present disclosure may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified (e.g., by an amino acid substitution) to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Transmembrane regions may be derived from (i.e. comprise) a receptor tyrosine kinase (e.g., ErbB2), glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 la, CD1 lb, CD1 lc, CD1 Id, CDS, CEACAM1, CRT AM, cytokine receptor, DAP-10, DNAM1 (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, suitable intracellular signaling domain include, but are not limited to, activating Macrophage/Myeloid cell receptors CSFR1, MYD88, CD14, TIE2, TLR4, CR3, CD64, TREM2, DAP10, DAP12, CD169, DECTIN1, CD206, CD47, CD163, CD36, MARCO, TIM4, MERTK, F4/80, CD91, C1QR, LOX-1, CD68, SRA, BAI-1, ABCA7, CD36, CD31, Lactoferrin, or a fragment, truncation, or combination thereof.

In some embodiments, a receptor tyrosine kinase may be derived from (e.g., comprise) Insulin receptor (InsR), Insulin-like growth factor I receptor (IGF1R), Insulin receptor-related receptor (IRR), platelet derived growth factor receptor alpha (PDGFRa), platelet derived growth factor receptor beta (PDGFRfi). KIT proto-oncogene receptor tyrosine kinase (Kit), colony stimulating factor 1 receptor (CSFR), fms related tyrosine kinase 3 (FLT3), fms related tyrosine kinase 1 (VEGFR-1), kinase insert domain receptor (VEGFR-2), fms related tyrosine kinase 4 (VEGFR-3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), protein tyrosine kinase 7 (CCK4), neurotrophic receptor tyrosine kinase 1 (trkA), neurotrophic receptor tyrosine kinase 2 (trkB), neurotrophic receptor tyrosine kinase 3 (trkC), receptor tyrosine kinase like orphan receptor 1 (ROR1), receptor tyrosine kinase like orphan receptor 2 (ROR2), muscle associated receptor tyrosine kinase (MuSK), MET proto-oncogene, receptor tyrosine kinase (MET), macrophage stimulating 1 receptor (Ron), AXL receptor tyrosine kinase (Axl), TYR03 protein tyrosine kinase (Tyro3), MER proto-oncogene, tyrosine kinase (Mer), tyrosine kinase with immunoglobulin like and EGF like domains 1 (TIE1), TEK receptor tyrosine kinase (TIE2), EPH receptor A1 (EphAl), EPH receptor A2 (EphA2), (EPH receptor A3) EphA3, EPH receptor A4 (EphA4), EPH receptor A5 (EphA5), EPH receptor A6 (EphA6), EPH receptor A7 (EphA7), EPH receptor A8 (EphA8), EPH receptor A10 (EphAlO), EPH receptor B1 (EphBl), EPH receptor B2 (EphB2), EPH receptor B3 (EphB3), EPH receptor B4 (EphB4), EPH receptor B6 (EphB6), ret proto oncogene (Ret), receptor-like tyrosine kinase (RYK), discoidin domain receptor tyrosine kinase 1 (DDR1), discoidin domain receptor tyrosine kinase 2 (DDR2), c-ros oncogene 1, receptor tyrosine kinase (ROS), apoptosis associated tyrosine kinase (Lmr1), lemur tyrosine kinase 2 (Lmr2), lemur tyrosine kinase 3 (Lmr3), leukocyte receptor tyrosine kinase (LTK), ALK receptor tyrosine kinase (ALK), or serine/threonine/tyrosine kinase 1 (STYK1).

iv. Costimulatory Domain

In certain embodiments, the CAR comprises a costimulatory domain. In some embodiments, the costimulatory domain comprises 4-1BB (CD137), CD28, or both, and/or an intracellular T cell signaling domain. In a preferred embodiment, the costimulatory domain is human CD28, human 4-1BB, or both, and the intracellular T cell signaling domain is human CD3 zeta (ζ). 4-1BB, CD28, CD3 zeta may comprise less than the whole 4-1BB, CD28 or CD3 zeta, respectively. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Amur. Rev. Pharmacol. Toxicol. 56:59-83 (2016).

In some embodiments, a costimulatory domain comprises the amino acid sequence of KRGRKKLLYIFKQPFMRPVQTTQEEDGCSCRFPEEEEGGCE (SEQ ID NO: 401)) or QVQLVQSGAEVEKPGASVKVSCKASGYTFTDYYMHWVRQAPGQGLEWMGWINPN SGGTNYAQKFQGRVTMTRDTSISTAYMELSRLRSDDTAVYYCASGWDFDYWGQGT LVTVSSGGGGSGGGGSGGGGSGGGGSDIVMTQSPSSLSASVGDRVTITCRASQSIRY YLSWYQQKPGKAPKLLIYTASILQNGVPSRFSGSGSGTDFTLTISSLQPEDFATYYCL QTYTTPDFGPGTKVEIK (SEQ ID NO: 402). See, e.g., PCT Application No. US2022/33091 (WO202261490), which is incorporated herein by reference in its entirety.

v. Intracellular Signaling Domain

The intracellular (signaling) domain of the engineered T cells disclosed herein may provide signaling to an activating domain, which then activates at least one of the normal effector functions of the immune cell. Effector function of a T cell, for example, may be cytolytic activity or helper activity including the secretion of cytokines.

In some embodiments, suitable intracellular signaling domain comprise, but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD 19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1 la, CD1 lb, CD1 lc, CD1 1d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Ly108, lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

CD3 is an element of the T cell receptor on native T cells, and has been shown to be an important intracellular activating element in CARs. In some embodiments, the CD3 is CD3 zeta. In some embodiments, the activating domain comprises an amino acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the following sequence: RVKFSRSADAPAYKQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 403). See, e.g., PCT Application No. US2022/33091, which is incorporated herein by reference in its entirety.

### 4. Production of Polynucleotides

### A. Linear Precursor RNA Preparation

The DNA templates provided herein can be made using standard techniques of molecular biology. For example, the various elements of the vectors provided herein can be obtained using recombinant methods, such as by screening cDNA and genomic libraries from cells, or by deriving the polynucleotides from a DNA template known to include the same.

The various elements of the DNA template provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al., Science (1984) 223:1299; and Jay et al., J. Biol. Chem. (1984) 259:631 1.

Thus, particular nucleotide sequences can be obtained from DNA template harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired DNA template elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239: 1534-1536), and enzymatic filling-in of gapped oligonucleotides using T4 DNA polymerase (Queen et al., Proc. Natl. Acad. Sci. USA (1989) 86: 10029-10033) can be used.

Transcription of a DNA template (e.g., comprising a 3′ intron element, 3′ exon element, an intervening region or core functional element including an IRES and expression sequence, a 5′ exon element, and a 5′ intron element) results in formation of a precursor linear RNA polynucleotide capable of circularizing. In some embodiments, this DNA template comprises a vector, PCR product, plasmid, minicircle DNA, cosmid, artificial chromosome, complementary DNA (cDNA), extrachromosomal DNA (ecDNA), or a fragment therein. In certain embodiments, the minicircle DNA may be linearized or non-linearized. In certain embodiments, the plasmid may be linearized or non-linearized. In some embodiments, the DNA template may be single-stranded. In other embodiments, the DNA template may be double-stranded. In some embodiments, the DNA template comprises in whole or in part from a viral, bacterial or eukaryotic vector. In some embodiments, the polynucleotide of the present disclosure is an expression vector.

The precursor RNA provided herein can be generated by incubating a DNA template provided herein under conditions permissive of transcription of the precursor RNA encoded by the DNA template. For example, in some embodiments a precursor RNA is synthesized by incubating a DNA template provided herein that comprises an RNA polymerase promoter or promoter segment upstream of its 5′ duplex sequence and/or expression sequences with a compatible RNA polymerase enzyme under conditions permissive of in vitro transcription. In some embodiments, the DNA template is incubated inside of a cell by a bacteriophage RNA polymerase or in the nucleus of a cell by host RNA polymerase II. In some embodiments, the polynucleotide of the present disclosure is an expression vector, wherein the expression vector comprises a polymerase promoter sequence or segment.

In certain embodiments, provided herein is a method of generating precursor RNA by performing in vitro transcription using a DNA template provided herein as a template (e.g., a vector provided herein with an RNA polymerase promoter or promoter segment positioned upstream of the 5′ duplex region).

In some embodiments, the DNA template shares the same sequence as the precursor linear RNA polynucleotide prior to splicing of the precursor linear RNA polynucleotide (e.g., a 3′ intron element, a 3′ exon element, an intervening region core functional element, and a 5′ exon element, a 5′ intron element). In some embodiments, said linear precursor RNA polynucleotide undergoes splicing leading to the removal of the 3′ intron element and 5′ intron element during the process of circularization. In some embodiments, the resulting circular RNA polynucleotide lacks a 3′ intron fragment and a 5′ intron fragment, but maintains a 3′ exon fragment, an intervening region or a core functional element, and a 5′ exon element.

In certain embodiments, the resulting precursor RNA can be used to generate circular RNA (e.g., a circular RNA polynucleotide provided herein) by incubating it in the presence of magnesium ions and guanosine nucleotide or nucleoside at a temperature at which RNA circularization occurs (e.g., between 20° C. and 60° C.). Precursor RNA are generally described in PCT Application No. US2022/33091, which is incorporated herein by reference in its entirety.

### B. Circular RNA Preparation

Thus, in certain embodiments provided herein is a method of making circular RNA. In certain embodiments, the method comprises synthesizing precursor RNA by transcription (e.g., run-off transcription) using a vector provided herein (e.g., a 5′ intron element, a 5′ exon element, an intervening region or core functional element, a 3′ exon element, and a 3′ intron element) as a template, and incubating the resulting precursor RNA in the presence of divalent cations (e.g., magnesium ions) and GTP such that it circularizes to form circular RNA. In some embodiments, the precursor RNA disclosed herein is capable of circularizing in the absence of magnesium ions and GTP and/or without the step of incubation with magnesium ions and GTP. In some embodiments, the precursor linear RNA polynucleotide circularizes when incubated in the presence of one or more guanosine nucleotides or nucleoside (e.g., GTP) and a divalent cation (e.g., Mg2+).

In certain embodiments, transcription occurs at a Mg2+ concentration of at least 3 mM of magnesium. In certain embodiments, the transcription occurs at a Mg2+ concentration of no more than 100 mM of magnesium. In certain embodiments, transcription occurs at a Mg2+ concentration of or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM. In some embodiments, the greater concentration of Mg2+ during transcription of a linear RNA polynucleotide improves circularization and/or splicing as compared to the same linear RNA polynucleotide undergoing transcription at a lower Mg2+ concentration. In some embodiments, the 3′ exon element, 5′ exon element, and/or core functional element in whole or in part promotes the circularization of the precursor linear RNA polynucleotide to form the circular RNA construct provided herein.

In other embodiments, the method comprises ligation. In some embodiments, the method comprises chemical ligation. In some embodiments, the method comprises splint mediated ligation. In some embodiments, the ligation is performed with a T4 ligase using splint DNA.

In some embodiments, the method of preparing a circular RNA comprises providing modified nucleotides or nucleosides to precursor RNA comprising:
(i) a 5′ combined accessory element, comprising:
   i. a 3′ intron segment,
   ii. a 3′ exon segment,
(ii) an intervening region comprising an internal ribosome entry site (IRES) and an expression sequence encoding a CD19 binding molecule,
(iii) a 3′ combined accessory element, comprising:
   i. a 5′ exon segment, and
   ii. a 5′ intron segment.

In some embodiments, the method of preparing a circular RNA comprises providing a first and second linear precursor RNA polynucleotide, wherein the first and second linear precursor RNA polynucleotides are capable of forming a circular RNA (e.g., ligation or permuted introns).

In some embodiments, a first precursor and a second precursor may be ligated to form a circular RNA. In some embodiments, the first precursor and the second precursor each comprise a short adapter sequence at their 5′ and 3′ ends. In some embodiments, the adapter sequences comprise homology arms with splints used for circularization. Splint ligation may be performed in the presence of a DNA splint using a suitable ligase to generate a circular RNA polynucleotide. Ligation methods are known in the art. See, e.g., Wesselhoeft et al., 2019.

In some embodiments, a first precursor and a second precursor may splice to form a circular RNA comprising a sequence of interest, e.g., a coding region. Each of the first precursor and the second precursor comprises at least one fragment of the sequence of interest, e.g., the first precursor comprises the 5′ fragment of the sequence of interest and the second precursor comprises the 3′ fragment of the sequence of interest. In these embodiments, the 5′ fragment of the sequence of interest, the 3′ fragment of the sequence of interest, and two additional fragments of the sequence of interest (Exon 2A, Exon 2B), together form the sequence of interest. In these embodiments, sequence of interest consists of, e.g., in 5′ to 3′ order, the 5′ fragment of the sequence of interest, an exonic fragment of the sequence of interest (Exon 2A), an exonic fragment of the sequence of interest (Exon 2B), and the 3′ fragment of the sequence of interest.

In some embodiments, the first precursor comprises the following:
(a) two intron fragments (e.g., 3′ intron fragment of a first intron (Intron 1) and a 5′ intron fragment of a second intron (Intron 2)),
(b) a translation initiation element (e.g., IRES),
(c) a 5′ fragment of the sequence of interest, and
(d) two exon fragments that correspond with the intron fragments (e.g., Exon 1B and Exon 2A).

In some embodiments, one exon fragment (e.g., Exon 2A) is a part of a sequence of interest, for example in the coding or noncoding region. The coding region is scanned for sequences that are homologous to this exon (Exon 2A) fragment, thereby allowing splicing to occur without altering the resulting coding sequence in the circular RNA.

In some embodiments, the second precursor comprises the following:
(a) two intron fragments that correspond with those in the first precursor (e.g., 3′ intron fragment of the second intron (Intron 2) and the 5′ intron fragment of the first intron (Intron 1)), and
(b) two exon fragments that correspond with those on the first precursor (e.g., Exon 2B, which corresponds to the 5′ fragment of the 3′ fragment of the sequence of interest (e.g., coding region) and Exon 1A), and
(c) the 3′ fragment of the sequence of interest.

In some embodiments, the coding sequence is scanned for regions that are homologous to this exon (Exon 2B) fragment, thereby allowing splicing to occur without altering the resulting coding sequence in the circular RNA. The first precursor and the second precursor may be incubated together to facilitate splicing between the first precursor and the second precursor in order to generate a circular RNA polynucleotide, which comprises specific modified regions and specific unmodified regions.

In some embodiments, a first precursor comprises an optional first external homology region (Arm 1A), a first intron fragment (3′ intron fragment of a first intron (Intron 1)), a first exon fragment (Exon 1B), an optional internal homology region, an optional spacer, a translation initiation element (e.g., IRES), the 5′ fragment of the sequence of interest (e.g., coding region), a second exon fragment (Exon 2A), a second intron fragment (5′ intron fragment of a second intron (Intron 2)), and an optional second external homology region (Arm 2A). In these embodiments, the second precursor comprises an optional first external homology region (Arm 2B), a first intron fragment (3′ intron fragment of the second intron (Intron 2)), a first exon fragment (Exon 2B corresponding to the 5′ fragment of the 3′ fragment of the sequence of interest (e.g., coding region)), the 3′ fragment of the sequence of interest (e.g., coding region), an optional spacer, an optional internal homology region, a second exon fragment (Exon 1A), a second intron fragment (5′ fragment of the first intron (Intron 1)), and an optional second external homology region (Arm 1B).

In some embodiments, either the first precursor or the second precursor comprises a monotron.

In some embodiments, the first precursor comprises an optional first external homology region (Arm 1A), a first intron fragment (3′ intron fragment of a first intron (Intron 1)), a first exon fragment (Exon 1B), an optional internal homology region, an optional spacer, a translation initiation element (e.g., IRES), the 5′ fragment of the sequence of interest (e.g., coding region), a second exon fragment (Exon 2A), a terminal element corresponding to a monotron sequence, and an optional second external homology region (Arm 2A). In these embodiments, the second precursor comprises an optional external homology region (Arm 2B), the monotron sequence via Intron 2, a first exon fragment (Exon 2B, which corresponds to the 5′ fragment of the 3′ fragment of the sequence of interest (e.g., coding region)), the 3′ fragment of the sequence of interest (e.g., coding region), an optional spacer, an optional internal homology region, a second exon fragment (Exon 1A), an intron fragment (5′ intron fragment of Intron 2), and an optional second external homology region (Arm 1B).

In some embodiments, the first precursor comprises an optional first external homology region (Arm 1A), a monotron sequence via Intron 1, a first exon fragment (Exon 1B), an optional internal homology region, an optional spacer, a translation initiation element (e.g., IRES), the 5′ fragment of the sequence of interest (e.g., coding region), a second exon fragment (Exon 2A), an intron fragment (5′ intron fragment of Intron 2), and an optional second external homology region. In these embodiments, the second precursor comprises an optional external homology region (Arm 2B), an intron fragment (3′ intron fragment of Intron 2), a first exon fragment (Exon 2B, which corresponds to the 5′ fragment of the 3′ fragment of the sequence of interest (e.g., coding region)), a 3′ fragment of the sequence of interest (e.g., coding region), an optional spacer, an optional internal homology region, a second exon fragment (Exon 1A), a terminal element corresponding to the monotron sequence, and an optional second external homology region (Arm 1B).

In some embodiments, each of the first precursor and the second precursor comprises a monotron. In some embodiments, the first precursor comprises an optional first external homology region (Arm 1A), a first monotron sequence via Intron 1, a first exon fragment (Exon 1B), an optional internal homology region, an optional spacer, a translation initiation element (e.g., IRES), the 5′ fragment of the sequence of interest, a second exon fragment (Exon 2A), a terminal element corresponding to a second monotron sequence via Intron 2, and an optional second external homology region (Arm 2A). In these embodiments, the second precursor comprises an optional external homology region (Arm 2B), a second monotron sequence via Intron 2, a first exon fragment (Exon 2B), the 3′ fragment of the sequence of interest (e.g., coding region), an optional spacer, an optional internal homology region, a second exon fragment (Exon 1A), a terminal element corresponding to the first monotron sequence via Intron 1, and an optional second external homology region (Arm 1B).

In some embodiments, provided herein are circular RNA that do not comprise modified nucleotides and/or modified nucleosides. Also provided herein are modified circular RNA (i.e., comprising at least one modified nucleotide and/or modified nucleoside) prepared from the methods described herein. In some embodiments, the modified circular RNA affects immunogenicity, circularization, and/or translation as compared to circular RNA prepared with RNA precursor polynucleotides that comprise no nucleotide or nucleoside modifications.

It has been discovered that circular RNA has reduced immunogenicity relative to a corresponding linear mRNA, at least partially because the linear mRNA contains an immunogenic 5′ cap. When transcribing a DNA vector from certain promoters (e.g., a T7 promoter, SP6 promoter, or a fragment thereof) to produce a precursor RNA, it is understood that the 5′ end of the precursor RNA is G. To reduce the immunogenicity of a circular RNA composition that contains a low level of contaminant linear mRNA, an excess of GMP relative to GTP can be provided during transcription such that most transcripts contain a 5′ GMP, which cannot be capped. Therefore, in some embodiments, transcription is carried out in the presence of an excess of GMP. In some embodiments, transcription is carried out where the ratio of GMP concentration to GTP concentration is within the range of about 3:1 to about 15:1, for example, about 3:1 to about 10:1, about 3:1 to about 5:1, about 3:1, about 4:1, or about 5:1.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, purification of circular RNA comprises an affinity-purification or negative selection method described herein. In some embodiments, purification of circular RNA comprises separation of linear RNA from circular RNA using oligonucleotides that are complementary to a sequence in the linear RNA but are not complementary to a sequence in the circular RNA. In some embodiments, a purified composition is less immunogenic than an unpurified composition. In some embodiments, immune cells exposed to a purified composition produce less TNFα, RIG-I, IL-2, IL-6, IFNγ, and/or a type 1 interferon, e.g., IFN-β1, than immune cells exposed to an unpurified composition.

In some embodiments, circular RNA is produced by transcribing a DNA polynucleotide sequence that is complementary to a precursory RNA polynucleotide that is described herein. In certain embodiments, circular RNA provided herein is produced in vitro. In certain embodiments, circular RNA provided herein is produced inside a cell. In some embodiments, the cell selected from, for example, an immune cell, muscle cell, neural cell, epithelial cell and a tumor cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

Exemplary methods of circularization of precursor RNA can be found in, for example, WO2020/237227, which is incorporated by reference herein in its entirety. WO2020/237227, inter alia, describes using the permuted intron exon (PIE) circularization strategy to circularize long precursor RNA. In it, a 1.1 kb sequence containing a full-length encephalomyocarditis virus (EMCV) IRES, a Gaussia luciferase (GLuc) expression sequence, and two short exon fragments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained. Perfectly complementary 9 nucleotide and 19 nucleotide long homology regions were designed and added at the 5′ and 3′ ends of the precursor RNA. The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. WO2020/237227 further indicates that a series of spacers was designed and inserted between the 3′ PIE splice site and the IRES. These spacers were designed to either conserve or disrupt secondary structures within intron sequences in the IRES, 3′ PIE splice site, and/or 5′ splice site.

Further methods for preparing circular RNA are described in PCT Application No. US2022/33091, which is incorporated herein by reference in its entirety.

### 5. Transfer Vehicle & Other Delivery Mechanisms

### A. Ionizable Lipids

In certain embodiments, disclosed herein are ionizable lipids that may be used as a component of a transfer vehicle to facilitate or enhance the delivery and release of circular RNA and/or linear mRNA to one or more target cells (e.g., by permeating or fusing with the lipid membranes of such target cells). In certain embodiments, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells.

In some embodiments, an ionizable lipid is as described in international patent application PCT/US2020/038678. In some embodiments, an ionizable lipid is a lipid as represented by formula 1 of or as listed in Tables 1 or 2 of U.S. Pat. No. 9,708,628, the content of which is herein incorporated by reference in its entirety. In some embodiments, an ionizable lipid is as described in pages 7-13 of U.S. Pat. No. 9,765,022 or as represented by formula 1 of U.S. Pat. No. 9,765,022, the content of which is herein incorporated by reference in its entirety. In some embodiments, an ionizable lipid is described in pages 12-24 of International Patent Application No. PCT/US2019/016362 or as represented by formula 1 of International Patent Application PCT/US2019/016362, the contents of which are herein incorporated by reference in their entirety. In some embodiments, a lipid or transfer vehicle is a lipid as described in International Patent Application Nos. PCT/US2010/061058, PCT/US2018/058555, PCT/US2018/053569, PCT/US2017/028981, PCT/US2019/025246, PCT/US2019/015913, PCT/US2019/016362, PCT/US2019/016362, US Application Publication Nos. US2019/0314524, US2019/0321489, US2019/0314284, and US2019/0091164, the contents of which are herein incorporated by reference in their entireties. Suitable cationic lipids for use in the compositions and methods herein include those described in international patent publication WO 2010/053572 and/or U.S. patent application Ser. No. 15/809,680, e.g., C12-200. In certain embodiments, the compositions and methods herein employ an ionizable cationic lipid described in WO2013149140 (incorporated herein by reference), such as, e.g., (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002). In certain embodiments, the compositions and methods herein employ an ionizable cationic lipid described US patent publications 2017/0190661 and 2017/0114010, incorporated herein by reference in their entirety.

In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP,” 1,2-Dioleoyl-3-Trimethylammonium-Propane or “DOTAP.” Contemplated cationic lipids also include 1,2-distearyloxy-N,N-dimethyl-3-aminopropane or “DSDMA”, 1,2-dioleyloxy-N,N-dimethyl-3-aminopropane or “DODMA,” 1,2-dilinoleyloxy-N,N-dimethyl-3-aminopropane or “DLinDMA,” 1,2-dilinolenyloxy-N,N-dimethyl-3-aminopropane or “DLenDMA,” N-dioleyl-N,N-dimethylammonium chloride or “DODAC,” N,N-distearyl-N,N-dimethylammonium bromide or “DDAB,” N-(1,2-dimyristyloxyprop-3-yl)-N,N-dimethyl-N-hydroxyethyl ammonium bromide or “DMRIE,” 3-dimethylamino-2-(cholest-5-en-3-beta-oxybutan-4-oxy)-1-(cis,cis-9,12-octadecadienoxy)propane or “CLinDMA,” 2-[5′-(cholest-5-en-3-beta-oxy)-3′-oxapentoxy)-3-dimethy 1-1-(cis,cis-9′, 1-2′-octadecadienoxy)propane or “CpLinDMA,” N,N-dimethyl-3,4-dioleyloxybenzylamine or “DMOBA,” 1,2-N,N′-dioleylcarbamyl-3-dimethylaminopropane or “DOcarbDAP,” 2,3-Dilinoleoyloxy-N,N-dimethylpropylamine or “DLinDAP,” 1,2-N,N′-Dilinoleylcarbamyl-3-dimethylaminopropane or “DLincarbDAP,” 1,2-Dilinoleoylcarbamyl-3-dimethylaminopropane or “DLinCDAP,” 2,2-dilinoleyl-4-dimethylaminomethyl-[1,3]-dioxolane or “DLin-K-DMA,” 2,2-dilinoleyl-4-dimethylaminoethyl-[1,3]-dioxolane or “DLin-K-XTC2-DMA,” and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1).

The use of cholesterol-based cationic lipids to formulate the transfer vehicles (e.g., lipid nanoparticles) is also contemplated herein. Such cholesterol-based cationic lipids can be used, either alone or in combination with other cationic or non-cationic lipids. Suitable cholesterol-based cationic lipids include, for example, GL67, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

In some embodiments, the one or more of the cationic or ionizable lipids provide increased activity of the nucleic acid and improved tolerability of the compositions in vivo.

PCT/US2022/033091 (WO 2022/261490) describes representative cationic lipids of any one of the disclosed embodiments and is incorporated by reference herein in its entirety.

In some embodiments, the cationic lipid (or a pharmaceutically acceptable salt, tautomer, prodrug or stereoisomer thereof) has a structure selected from one of the following that are described in detail on pages 113-118 of WO 2022/261490 and page 113-118 of WO 2023/056033, which are incorporated by reference herein in their entireties:

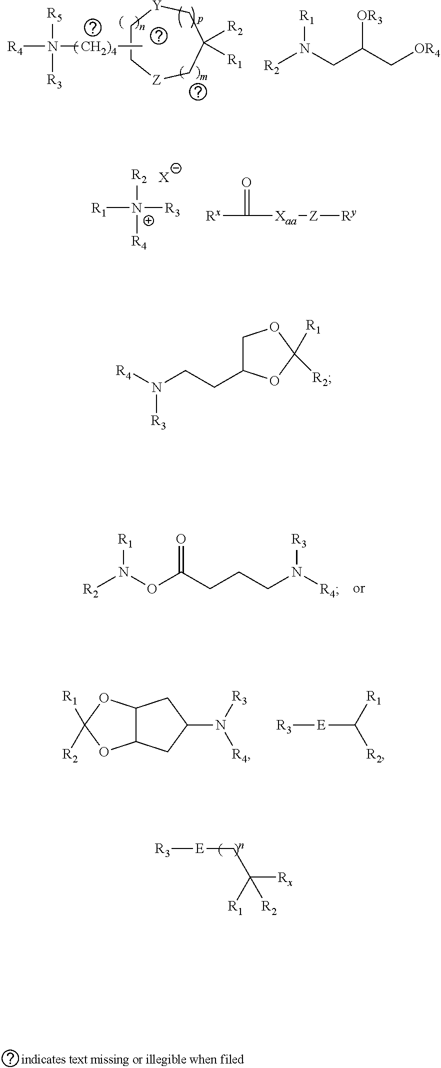

In some embodiments, for example, a cationic lipid of any one of the disclosed embodiments has a structure of Formula I and/or is selected from structure numbers I-1 through I-41, set forth at pages 119-130 and in Table 1 of WO 2022/261490; has a structure of Formula II and/or is selected from structure numbers II-1 through II-46, set forth at pages 130-146 and in Table 2 of WO 2022/261490; has a structure of Formula III and/or is selected from structure numbers III-1 through III-49, set forth at pages 146-157 and in Table 3 of WO 2022/261490; has a structure of Formula IV or V and/or is selected from structure numbers IV-1 through IV-3, set forth at pages 157-174 and in Table 4 of WO 2022/261490; has a structure of Formula VI and/or is selected from structure numbers VI-1 through VI-37, set forth at pages 174-188 and in Table 5 of WO 2022/261490; has a structure of Formula VII and/or is selected from structure numbers VII-1 through VII-11, set forth at pages 188-195 in Table 6 of WO 2022/261490; has a structure of Formula VIII and/or is selected from structure numbers VIII-1 through VII-12, set forth at pages 195-201 and in Table 7 of WO 2022/261490; has a structure of Formula IX and/or is selected from structure numbers IX-1 through IX-18, set forth at pages 201-208 and in Table 8 of WO 2022/261490; has a structure of Formula X and/or is selected from structure numbers X-1 through X-17, set forth at pages 208-213 and in Table 6 of WO 2022/261490; has a structure of Formula XI and/or is structure number XIa or Formula XII and/or is selected from structure numbers XIIA-XIIJ, as described at pages 213-220 in WO 2022/261490. WO 2023/056033 describes similar structures, and is incorporated by reference in its entirety.

In some embodiments, an ionizable lipid is a compound of Formula (1), Formula (1-1), Formula (1-2), Formula (2), Formula (3), Formula (3-1), Formula (3-2), Formula (3-3), Formula (5), or Formula (6), in WO 2022/261490, which is incorporated by reference herein in its entirety. WO 2022/261490 provides exemplary reaction schemes that illustrate an exemplary method to make compounds of Formula (1). WO 2023/056033 describes similar structures, and is incorporated by reference in its entirety.

In some embodiments, the ionizable lipid has a beta-hydroxyl amine head group. In some embodiments, the ionizable lipid has a gamma-hydroxyl amine head group.

In some embodiments, an ionizable lipid of the disclosure is a lipid selected from Table 10a, Table 10b, or Table 10 on pages 235-271—of WO 2022/261490, which is incorporated herein by reference in its entirety. In some embodiments, the ionizable lipid is Lipid 26, 27, 53, 54, 45, 46, 137, 138, 139, 128, or 130 in Table 10a of WO 2022/261490. In some embodiments, an ionizable lipid of the disclosure is Lipid 15 from Table 10b of WO 2022/261490.

In an embodiment, the ionizable lipid is described in US patent publication number US20170210697A1. In an embodiment, the ionizable lipid is described in US patent publication number US20170119904A1.

In some embodiments, the ionizable lipid has one of the structures set forth in Table 11 of WO 2022/261490, which is incorporated herein by reference in its entirety, certain of which are described in international patent application PCT/US2010/061058. In some embodiments, the ionizable lipids may include a lipid selected from Tables 12, 13, 14, or 15a of WO 2022/261490.

In some embodiments, the transfer vehicle comprises Lipid A, Lipid B, Lipid C, and/or Lipid D, described in detail, including methods of synthesis that are known in the art, in WO 2022/261490, WO 2023/056033, and PCT/US2017/028981, which are incorporated herein by reference in their entireties.

Also contemplated are ionizable lipids such as the dialkylamino-based, imidazole-based, and guanidinium-based lipids. See, e.g., PCT/US2010/058457, incorporated herein by reference. For example, certain embodiments are directed to a composition comprising one or more imidazole-based ionizable lipids, for example, the imidazole cholesterol ester or “ICE” lipid, (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII) of WO 2022/261490 and WO 2023/056033, which are incorporated herein by reference in their entireties. In an embodiment, a transfer vehicle for delivery of circRNA may comprise one or more imidazole-based ionizable lipids, for example, the imidazole cholesterol ester or “ICE” lipid (3S, 10R, 13R, 17R)-10, 13-dimethyl-17-((R)-6-methylheptan-2-yl)-2, 3, 4, 7, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII). In some embodiments, an ionizable lipid is described by US patent publication number 20190314284.

In certain embodiments, the ionizable lipid is described by structure (XIV), structure XVII (referred to herein as “HGT4001”), structure XVIII (referred to herein as “HGT4002”), structure XIX (referred to herein as “HGT4003”), structure XX (referred to herein as “HGT4004”), or structure XXI (referred to herein as “HGT4005”) of WO 2022/261490 and WO 2023/056033, which are incorporated herein by reference in their entireties.

In some embodiments, the ionizable lipid is selected from a lipid with a structure depicted on 390-457 of WO 2022/261490, which is incorporated herein by reference in its entirety.

WO 2023/056033 also describes representative cationic lipids of any one of the disclosed embodiments and is incorporated by reference herein in its entirety. In some embodiments, for example, a cationic lipid of any one of the disclosed embodiments has the structure of Formula (7), (7-1), (7-2), (7-3), (8), (8-1), (8-2), (8-3), (8-4), (9), (10), (11), and/or (12) of WO 2023/056033. In some embodiments, the cationic lipid is selected from a lipid with a structure depicted in any of Tables 10a-10f, Table 11, Tables 12, 13, 14, or 15a of WO 2023/056033. In some embodiments, the ionizable lipid is described by structure (XIV) of WO 2023/056033, and pharmaceutical compositions comprising the compound of structure XIV are envisioned. In some embodiments, the cationic lipid is selected from a lipid with a structure depicted on pages 386-439 of WO 2023/056033.

In some embodiments, the one or more of the cationic or ionizable lipids are represented by Formula (I):

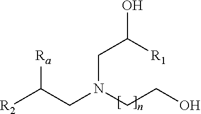

wherein:
   n is an integer between 1 and 4;
   Rₐ is hydrogen or hydroxyl; and
   R₁ and R₂ are each independently a linear or branched C₆-C₃₀ alkyl, C₆-C₃₀ alkenyl, or C₆-C₃₀ heteroalkyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

In some embodiments, Rₐ is hydrogen. In some embodiments, Rₐ is hydroxyl.

In some embodiments, the ionizable lipid is represented by Formula (Ia-1), Formula (Ia-2), or Formula (Ia-3):

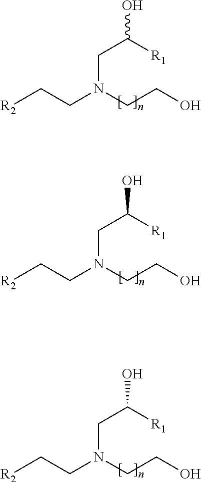

In some embodiments, the ionizable lipid is represented by Formula (Ib-1), Formula (Ib-2), or Formula (Ib-3):

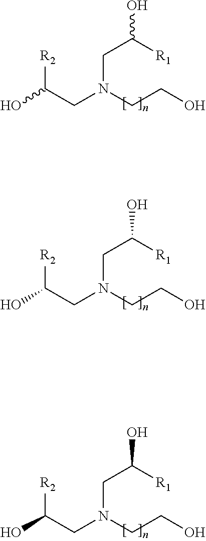

In some embodiments, the ionizable lipid is represented by Formula (Ib-4), Formula (Ib-5), Formula (Ib-6), Formula (Ib-7), Formula (Ib-8), or Formula (Ib-9):

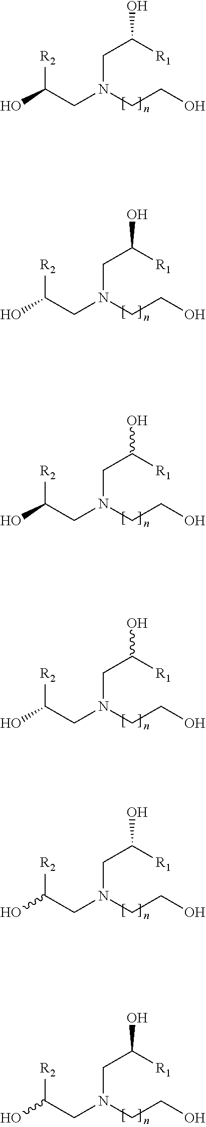

In some embodiments, the one or more of the cationic or ionizable lipids are represented by Formula (I), wherein R₁ and R₂ are each independently selected from:

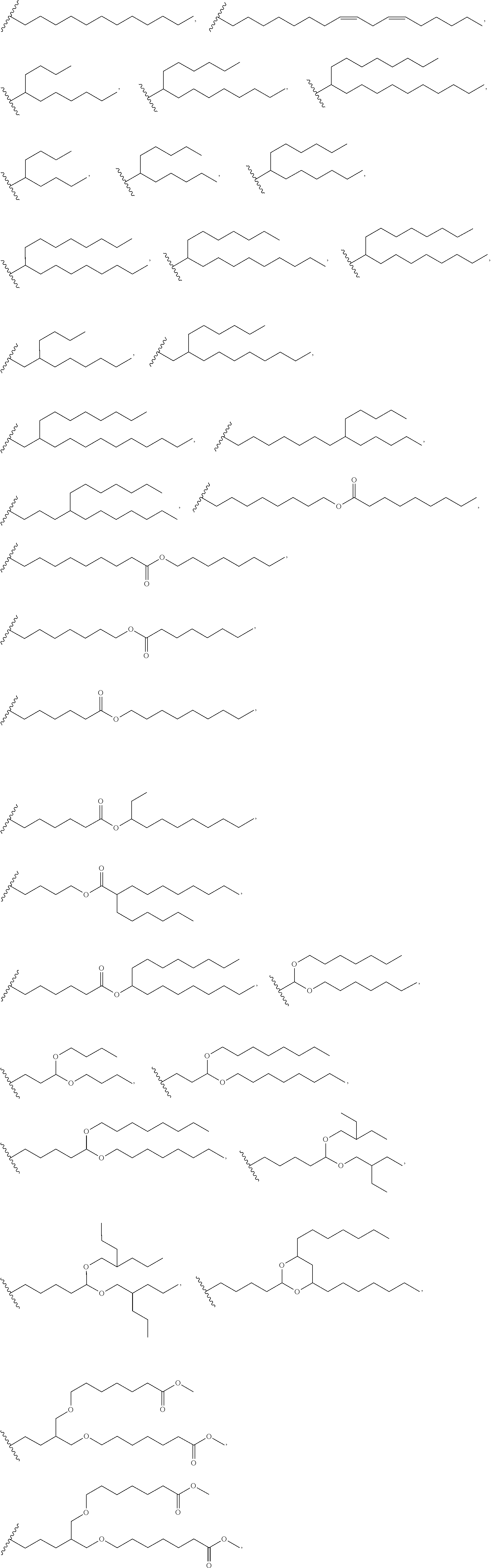

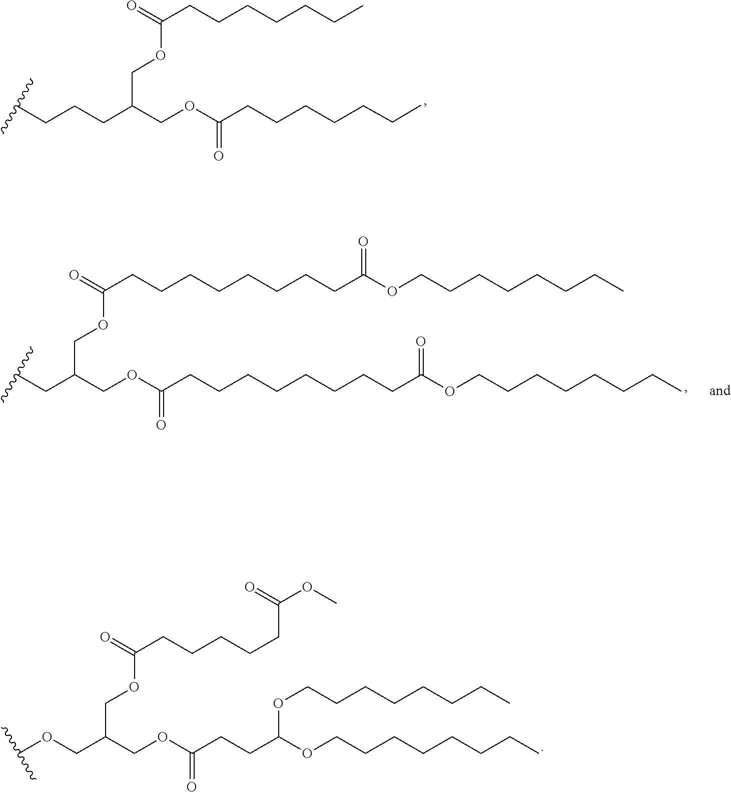 [SMILES: *CCCC(COC(=O)CCCCCCC)COC(=O)CCCCCCC ; *CC(COC(=O)CCCCCCCCC(=O)OCCCCCCCC)COC(=O)CCCCCCCCC(=O)OCCCCCCCC ; *OCC(COC(=O)CCCCCC(=O)OC)COC(=O)CCC(OCCCCCCCC)OCCCCCCCC]

In some embodiments, R₁ and R₂ are the same. In some embodiments, R₁ and R₂ are different.

In various embodiments, the one or more of the cationic or ionizable lipids are represented by Formula (I*):

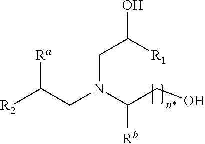

wherein:
   n* is an integer between 1 to 7,
   R^{a} is hydrogen or hydroxyl,
   R^{b} is hydrogen or C₁-C₆ alkyl,
   R₁ and R₂ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

In some embodiments, the one or more of the cationic or ionizable lipids are represented by Formula (II):

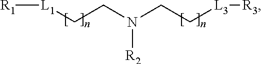

wherein:
each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl; and
R₂ is selected from a group consisting of:

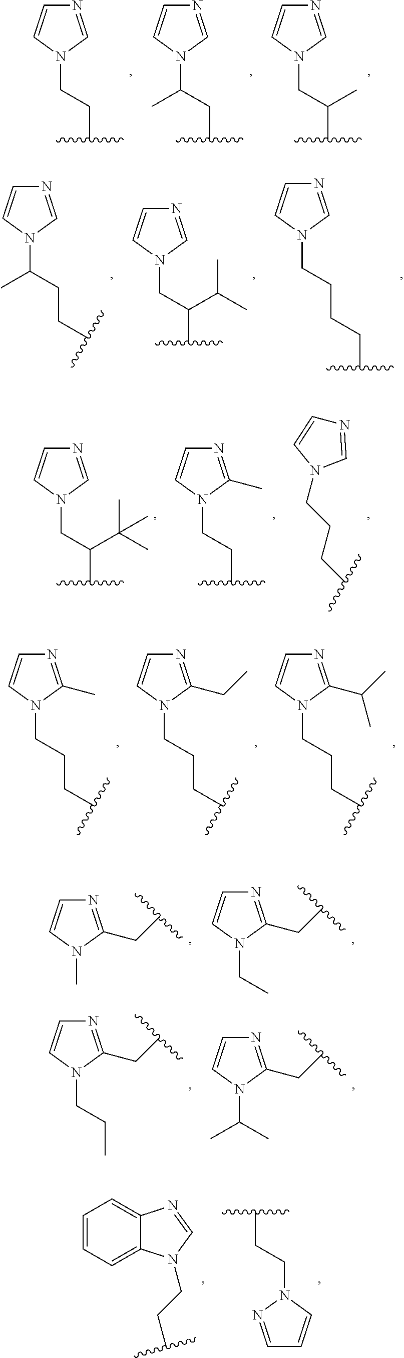

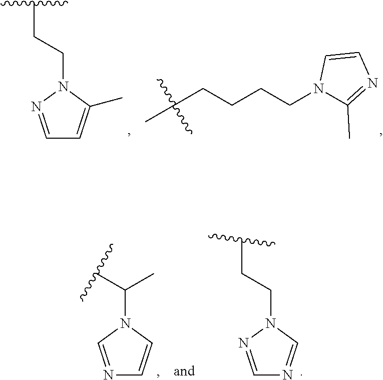

In some embodiments, the ionizable lipid is selected from an ionizable lipid of Formula II, wherein R₁ and R₃ are each independently selected from a group consisting of:

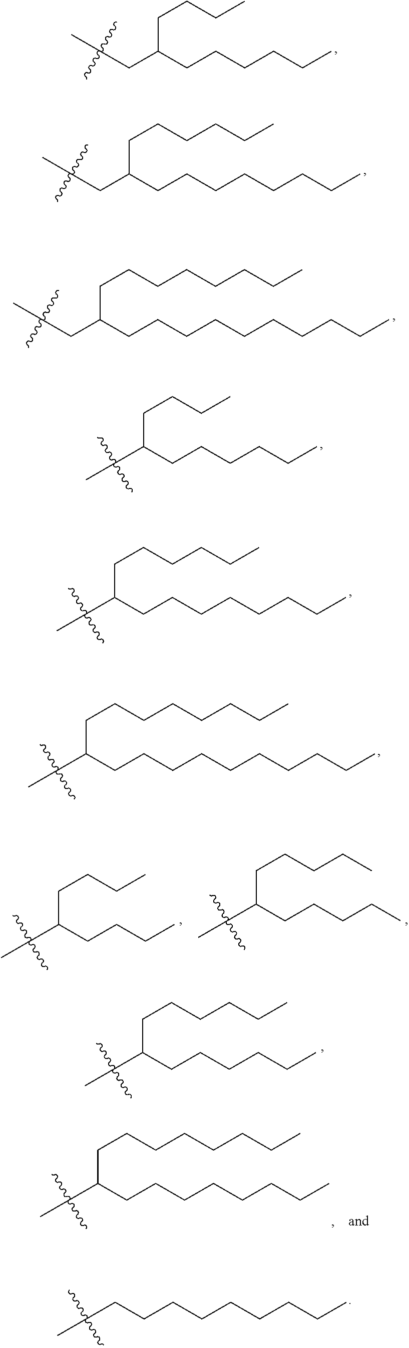

In some embodiments, R₁ and R₃ are the same. In some embodiments, R₁ and R₃ are different.

In some embodiments, the one or more of the cationic or ionizable lipids are represented by Formula (II-1) or Formula (II-2):

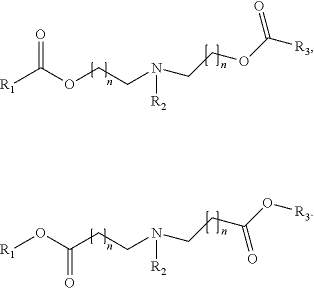

In some embodiments, the ionizable lipid is selected from an ionizable lipid of WO2015/095340 (lipid number 123 of Table 8 herein). In some embodiments, the ionizable lipid is selected from an ionizable lipid of WO2021/021634, WO2020/237227, or WO2019/236673 (lipid numbers 124-127 of Table 8 herein). In some embodiments, the ionizable lipid is selected from an ionizable lipid of WO2021226597 and WO2021113777 (lipid numbers 128-131 of Table 8 herein).

In some embodiments, the transfer vehicle comprises an ionizable lipid selected from an ionizable lipid represented in Table 8. In particular embodiments, where the ionizable lipid is of Formula I, the ionizable lipid is selected from lipid numbers 16, 45, 86, 89, and 90 of Table 8, below. In particular embodiments where the ionizable lipid is an ionizable lipid of Formula II, the ionizable lipid is selected from lipid numbers 128-131 of Table 8, below.

In some embodiments, the one or more of the cationic or ionizable lipids are represented by Formula (III):

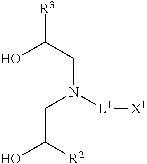

or a pharmaceutically acceptable salt thereof, wherein
L¹ is C₂-C₁₁ alkylene, C₄-C₁₀-alkenylene, or C₄-C₁₀-alkynylene;
X¹ is OR¹, SR¹, or N(R¹)₂, where R¹ is independently H or unsubstituted C₁-C₆ alkyl; and
R² and R³ are each independently C₆-C₃₀-alkyl, C₆-C₃₀-alkenyl, or C₆-C₃₀-alkynyl.

In some embodiments, the one or more of the cationic or ionizable lipids are represented by Formula (III*):

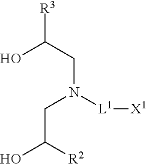

or a pharmaceutically acceptable salt thereof, wherein
   L¹ is C₂-C₁₁ alkylene, C₄-C₁₀-alkenylene, or C₄-C₁₀-alkynylene;
   X¹ is OR¹, SR¹, or N(R¹)₂, where R¹ is independently H or unsubstituted C₁-C₆ alkyl; and
   R₂ and R₃ are each independently a linear or branched C₁-C₃₀ alkyl, C₂-C₃₀ alkenyl, or C₁-C₃₀ heteroalkyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, alkylcarbonyloxy, alkylcarbonate, alkenyloxycarbonyl, alkenylcarbonyloxy, alkenylcarbonate, alkynyloxycarbonyl, alkynylcarbonyloxy, alkynylcarbonate, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl, and alkylsulfonealkyl.

TABLE 8
Exemplary Ionizable Lipid Structures

| Ionizable | |
|---|---|
| lipid | |
| number | Structure |
| 1 | 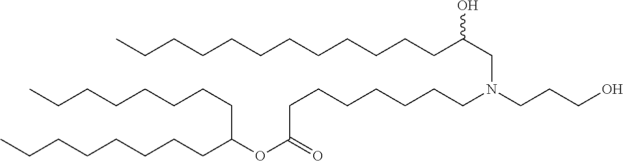 [SMILES: CCCCCCCCCCCCC(O)CN(CCCO)CCCCCCCC(=O)OC(CCCCCCCC)CCCCCCCC] |
| 2 | 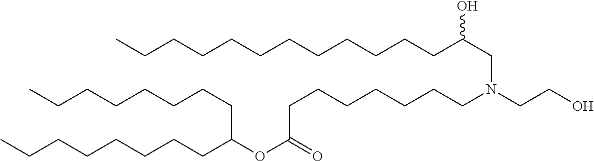 [SMILES: CCCCCCCCCCCCC(O)CN(CCO)CCCCCCCC(=O)OC(CCCCCCCC)CCCCCCCC] |
| 3 | 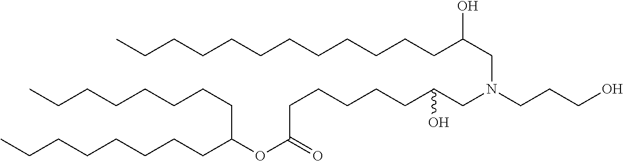 [SMILES: CCCCCCCCCCCCC(O)CN(CCCO)CC(O)CCCCCC(=O)OC(CCCCCCCC)CCCCCCCC] |
| 4 | 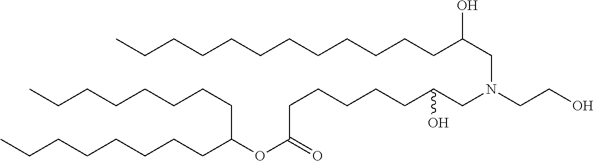 [SMILES: CCCCCCCCCCCCC(O)CN(CCO)CC(O)CCCCCC(=O)OC(CCCCCCCC)CCCCCCCC] |
| 5 | 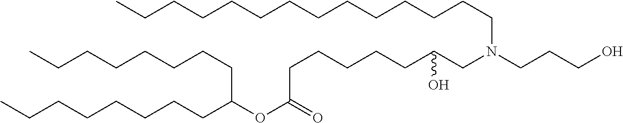 [SMILES: CCCCCCCCCCCCCCN(CCCO)CC(O)CCCCCC(=O)OC(CCCCCCCC)CCCCCCCC] |
| 6 | 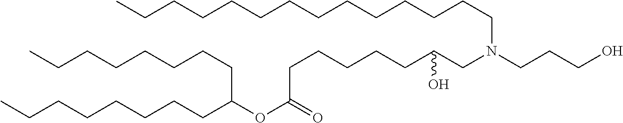 [SMILES: CCCCCCCCCCCCCCN(CCCO)CC(O)CCCCCC(=O)OC(CCCCCCCC)CCCCCCCC] |
| 7 | 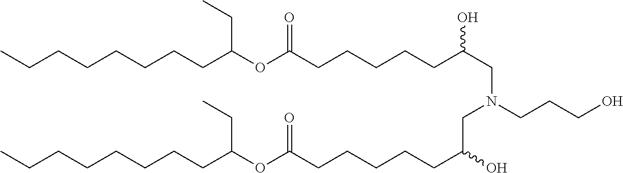 [SMILES: CCCCCCCCC(CC)OC(=O)CCCCCC(O)CN(CCCO)CC(O)CCCCCC(=O)OC(CC)CCCCCCCC] |
| 8 | 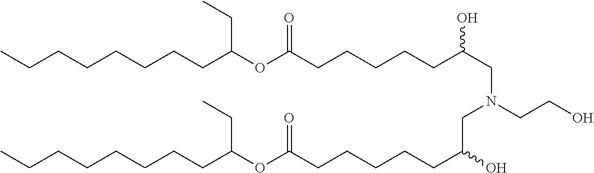 [SMILES: CCCCCCCCC(CC)OC(=O)CCCCCC(O)CN(CCO)CC(O)CCCCCC(=O)OC(CC)CCCCCCCC] |
| 9 | 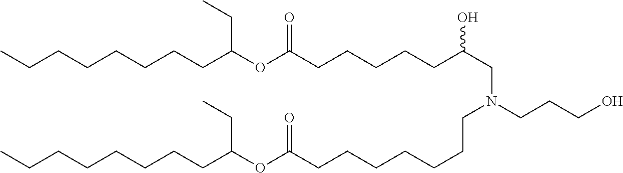 [SMILES: CCCCCCCCC(CC)OC(=O)CCCCCCCN(CCCO)CC(O)CCCCCC(=O)OC(CC)CCCCCCCC] |
| 10 | 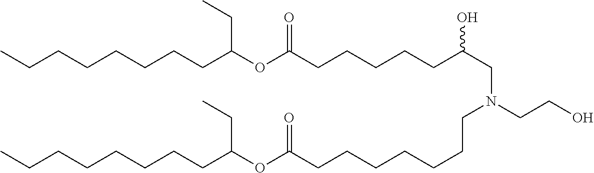 [SMILES: CCCCCCCCC(CC)OC(=O)CCCCCCCN(CCO)CC(O)CCCCCC(=O)OC(CC)CCCCCCCC] |
| 11 | 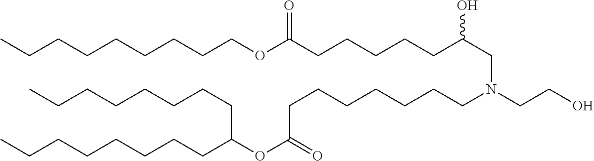 [SMILES: CCCCCCCCCOC(=O)CCCCCC(O)CN(CCO)CCCCCCCC(=O)OC(CCCCCCCC)CCCCCCCC] |
| 12 | 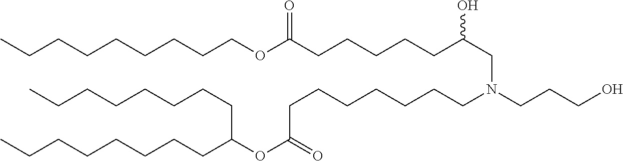 [SMILES: CCCCCCCCCOC(=O)CCCCCC(O)CN(CCCO)CCCCCCCC(=O)OC(CCCCCCCC)CCCCCCCC] |
| 13 | 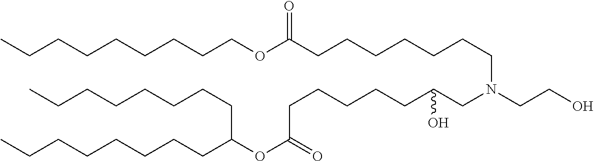 [SMILES: CCCCCCCCCOC(=O)CCCCCCCN(CCO)CC(O)CCCCCC(=O)OC(CCCCCCCC)CCCCCCCC] |
| 14 | 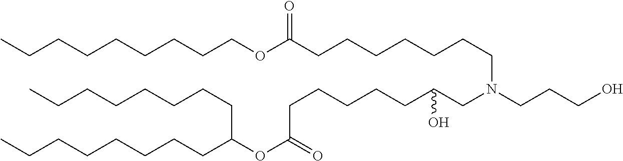 [SMILES: CCCCCCCCCOC(=O)CCCCCCCN(CCCO)CC(O)CCCCCC(=O)OC(CCCCCCCC)CCCCCCCC] |
| 15 | 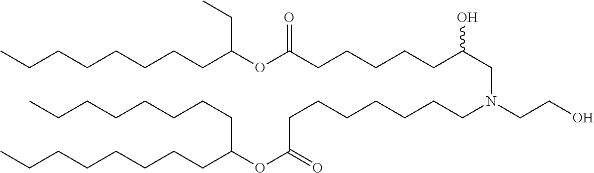 [SMILES: CCCCCCCCC(CC)OC(=O)CCCCCC(O)CN(CCO)CCCCCCCC(=O)OC(CCCCCCCC)CCCCCCCC] |
| 16 | 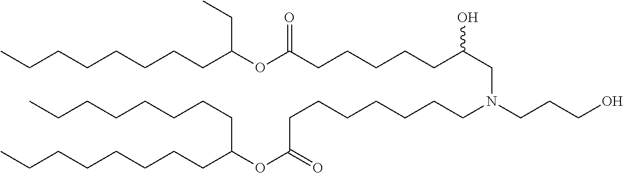 [SMILES: CCCCCCCCC(CC)OC(=O)CCCCCC(O)CN(CCCO)CCCCCCCC(=O)OC(CCCCCCCC)CCCCCCCC] |
| 17 | 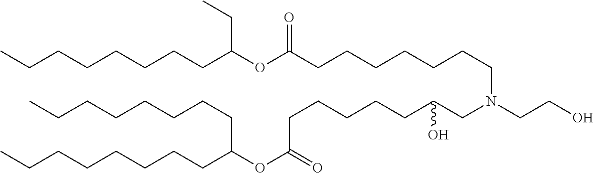 [SMILES: CCCCCCCCC(CC)OC(=O)CCCCCCCN(CCO)CC(O)CCCCCC(=O)OC(CCCCCCCC)CCCCCCCC] |
| 18 | 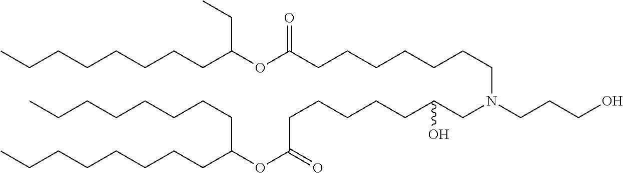 [SMILES: CCCCCCCCC(CC)OC(=O)CCCCCCCN(CCCO)CC(O)CCCCCC(=O)OC(CCCCCCCC)CCCCCCCC] |
| 19 | 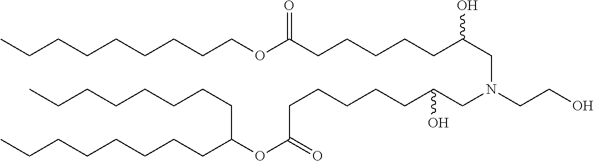 [SMILES: CCCCCCCCCOC(=O)CCCCCC(O)CN(CCO)CC(O)CCCCCC(=O)OC(CCCCCCCC)CCCCCCCC] |
| 20 | 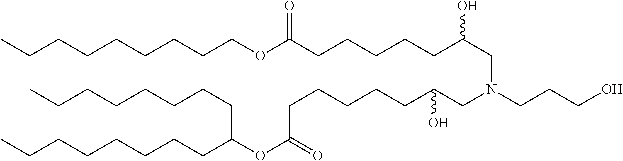 [SMILES: CCCCCCCCCOC(=O)CCCCCC(O)CN(CCCO)CC(O)CCCCCC(=O)OC(CCCCCCCC)CCCCCCCC] |
| 21 | 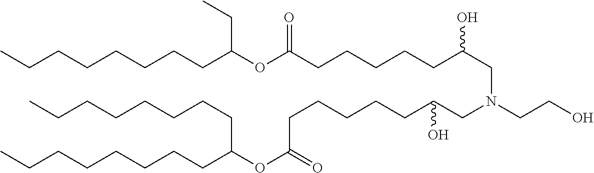 [SMILES: CCCCCCCCC(CC)OC(=O)CCCCCC(O)CN(CCO)CC(O)CCCCCC(=O)OC(CCCCCCCC)CCCCCCCC] |
| 22 | 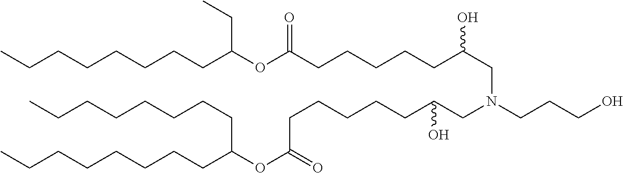 [SMILES: CCCCCCCCC(CC)OC(=O)CCCCCC(O)CN(CCCO)CC(O)CCCCCC(=O)OC(CCCCCCCC)CCCCCCCC] |
| 23 | 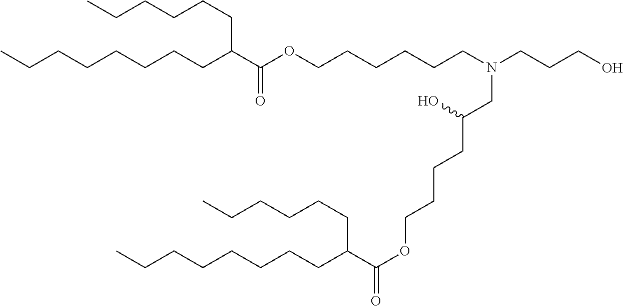 [SMILES: CCCCCCCCC(CCCCCC)C(=O)OCCCCCCN(CCCO)CC(O)CCCCOC(=O)C(CCCCCC)CCCCCCCC] |
| 24 | 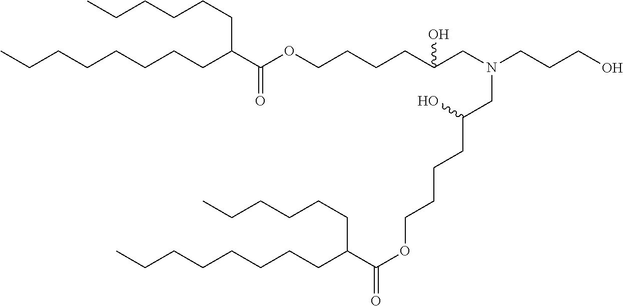 [SMILES: CCCCCCCCC(CCCCCC)C(=O)OCCCCC(O)CN(CCCO)CC(O)CCCCOC(=O)C(CCCCCC)CCCCCCCC] |
| 25 | 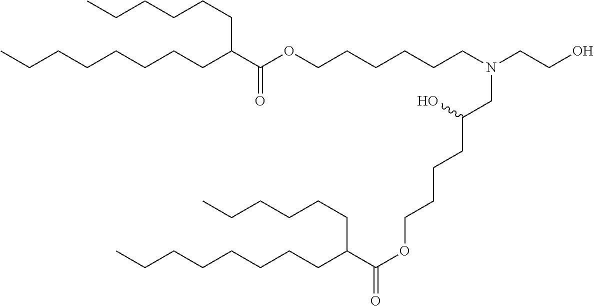 [SMILES: CCCCCCCCC(CCCCCC)C(=O)OCCCCCCN(CCO)CC(O)CCCCOC(=O)C(CCCCCC)CCCCCCCC] |
| 26 | 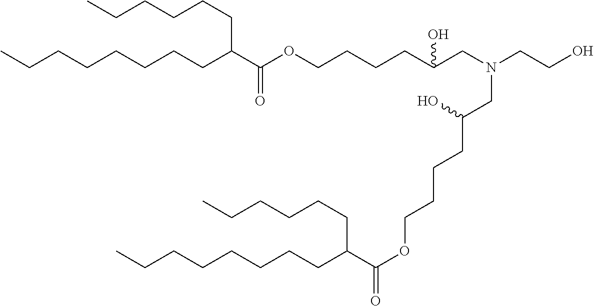 [SMILES: CCCCCCCCC(CCCCCC)C(=O)OCCCCC(O)CN(CCO)CC(O)CCCCOC(=O)C(CCCCCC)CCCCCCCC] |
| 27 | 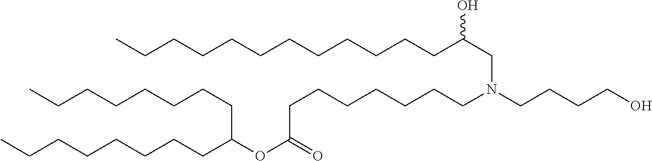 [SMILES: CCCCCCCCCCCCC(O)CN(CCCCO)CCCCCCCC(=O)OC(CCCCCCCC)CCCCCCCC] |
| 28 | 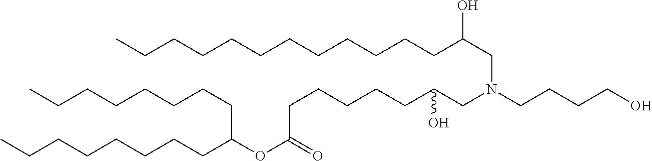 [SMILES: CCCCCCCCCCCCC(O)CN(CCCCO)CC(O)CCCCCC(=O)OC(CCCCCCCC)CCCCCCCC] |
| 29 | 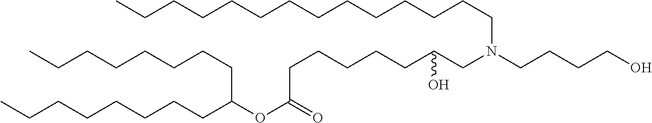 [SMILES: CCCCCCCCCCCCCCN(CCCCO)CC(O)CCCCCC(=O)OC(CCCCCCCC)CCCCCCCC] |
| 30 | 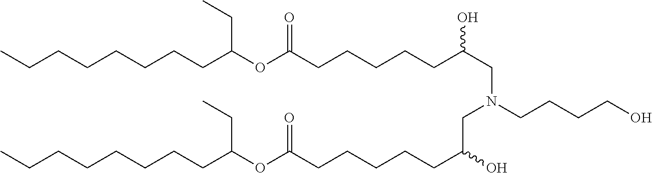 [SMILES: CCCCCCCCC(CC)OC(=O)CCCCCC(O)CN(CCCCO)CC(O)CCCCCC(=O)OC(CC)CCCCCCCC] |
| 31 | 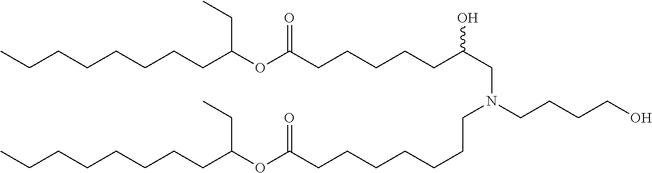 [SMILES: CCCCCCCCC(CC)OC(=O)CCCCCCCN(CCCCO)CC(O)CCCCCC(=O)OC(CC)CCCCCCCC] |
| 32 | 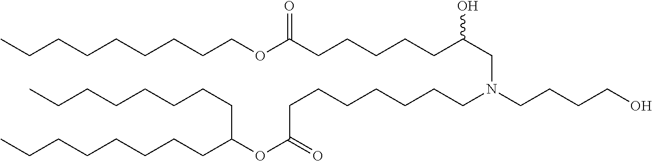 [SMILES: CCCCCCCCCOC(=O)CCCCCC(O)CN(CCCCO)CCCCCCCC(=O)OC(CCCCCCCC)CCCCCCCC] |
| 33 | 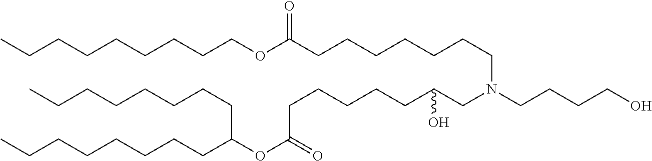 [SMILES: CCCCCCCCCOC(=O)CCCCCCCN(CCCCO)CC(O)CCCCCC(=O)OC(CCCCCCCC)CCCCCCCC] |
| 34 | 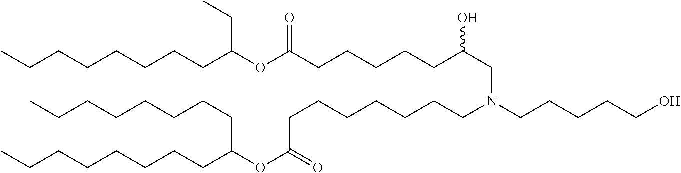 [SMILES: CCCCCCCCC(CC)OC(=O)CCCCCC(O)CN(CCCCCO)CCCCCCCC(=O)OC(CCCCCCCC)CCCCCCCC] |
| 35 | 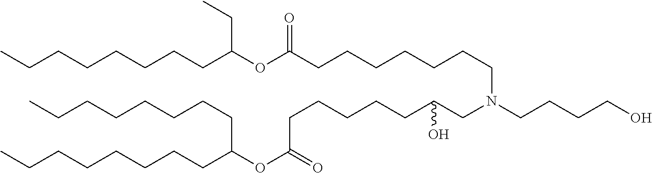 [SMILES: CCCCCCCCC(CC)OC(=O)CCCCCCCN(CCCCO)CC(O)CCCCCC(=O)OC(CCCCCCCC)CCCCCCCC] |
| 36 | 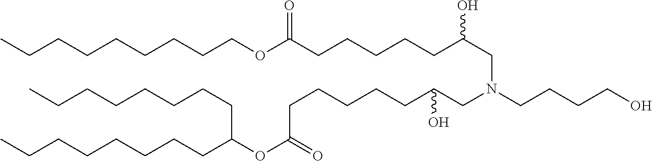 [SMILES: CCCCCCCCCOC(=O)CCCCCC(O)CN(CCCCO)CC(O)CCCCCC(=O)OC(CCCCCCCC)CCCCCCCC] |
| 37 | 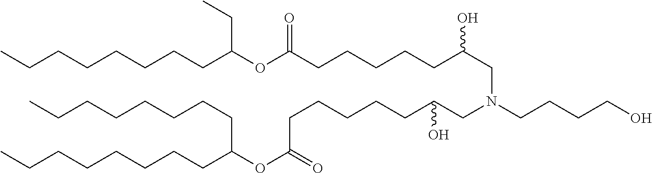 [SMILES: CCCCCCCCC(CC)OC(=O)CCCCCC(O)CN(CCCCO)CC(O)CCCCCC(=O)OC(CCCCCCCC)CCCCCCCC] |
| 38 | 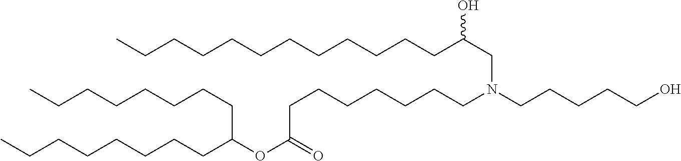 [SMILES: CCCCCCCCCCCCC(O)CN(CCCCCO)CCCCCCCC(=O)OC(CCCCCCCC)CCCCCCCC] |
| 39 | 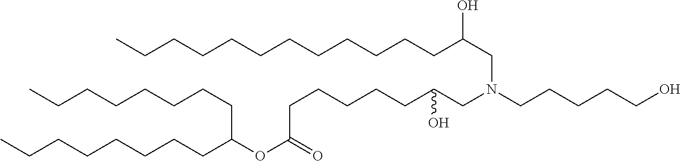 [SMILES: CCCCCCCCCCCCC(O)CN(CCCCCO)CC(O)CCCCCC(=O)OC(CCCCCCCC)CCCCCCCC] |
| 40 | 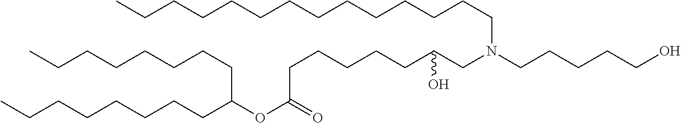 [SMILES: CCCCCCCCCCCCCCN(CCCCCO)CC(O)CCCCCC(=O)OC(CCCCCCCC)CCCCCCCC] |
| 41 | 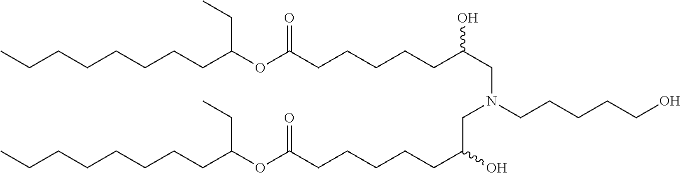 [SMILES: CCCCCCCCC(CC)OC(=O)CCCCCC(O)CN(CCCCCO)CC(O)CCCCCC(=O)OC(CC)CCCCCCCC] |
| 42 | 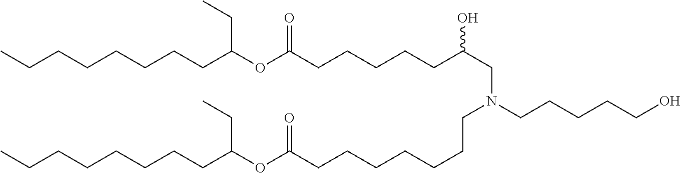 [SMILES: CCCCCCCCC(CC)OC(=O)CCCCCCCN(CCCCCO)CC(O)CCCCCC(=O)OC(CC)CCCCCCCC] |
| 43 | 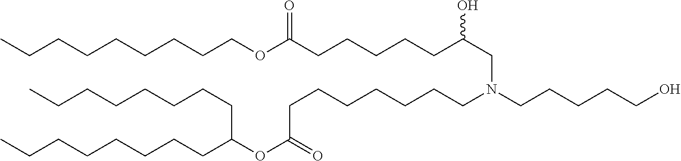 [SMILES: CCCCCCCCCOC(=O)CCCCCC(O)CN(CCCCCO)CCCCCCCC(=O)OC(CCCCCCCC)CCCCCCCC] |
| 44 | 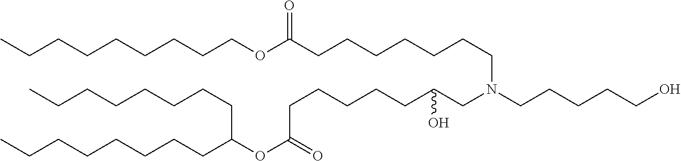 [SMILES: CCCCCCCCCOC(=O)CCCCCCCN(CCCCCO)CC(O)CCCCCC(=O)OC(CCCCCCCC)CCCCCCCC] |
| 45 | 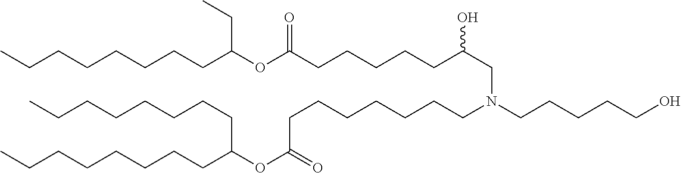 [SMILES: CCCCCCCCC(CC)OC(=O)CCCCCC(O)CN(CCCCCO)CCCCCCCC(=O)OC(CCCCCCCC)CCCCCCCC] |
| 46 | 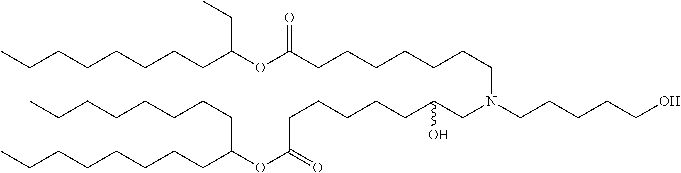 [SMILES: CCCCCCCCC(CC)OC(=O)CCCCCCCN(CCCCCO)CC(O)CCCCCC(=O)OC(CCCCCCCC)CCCCCCCC] |
| 47 | 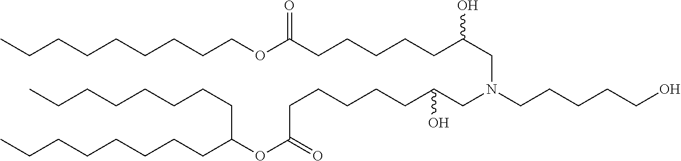 [SMILES: CCCCCCCCCOC(=O)CCCCCC(O)CN(CCCCCO)CC(O)CCCCCC(=O)OC(CCCCCCCC)CCCCCCCC] |
| 48 | 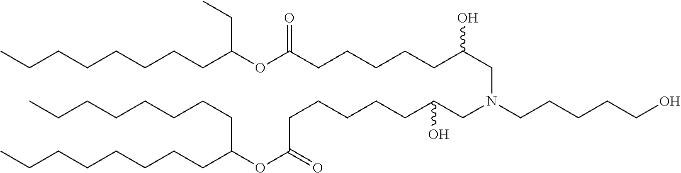 [SMILES: CCCCCCCCC(CC)OC(=O)CCCCCC(O)CN(CCCCCO)CC(O)CCCCCC(=O)OC(CCCCCCCC)CCCCCCCC] |
| 49 | 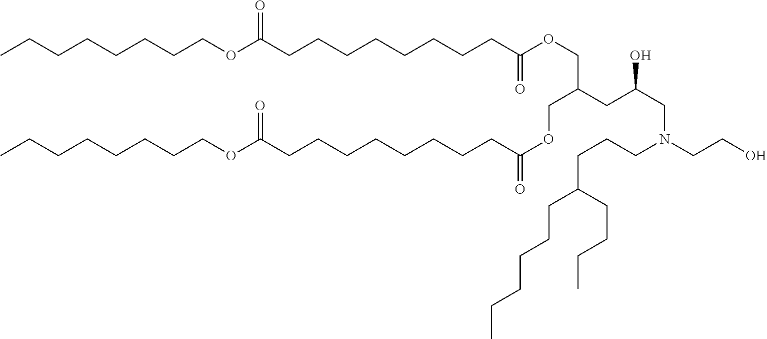 [SMILES: CCCCCCCCOC(=O)CCCCCCCCC(=O)OCC(COC(=O)CCCCCCCCC(=O)OCCCCCCCC)C[C@@H](O)CN(CCO)CCCC(CCCC)CCCCCC] |
| 50 | 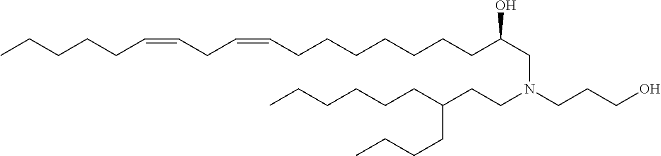 [SMILES: CCCCC/C=C\C/C=C\CCCCCCC[C@@H](O)CN(CCCO)CCC(CCCC)CCCCCC] |
| 51 | 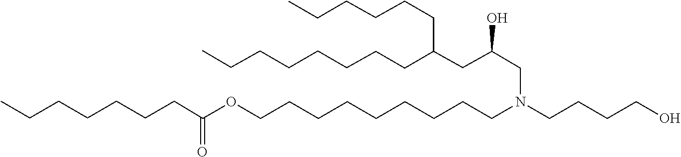 [SMILES: CCCCCCCCC(CCCCCC)C[C@@H](O)CN(CCCCO)CCCCCCCCCOC(=O)CCCCCCC] |
| 52 | 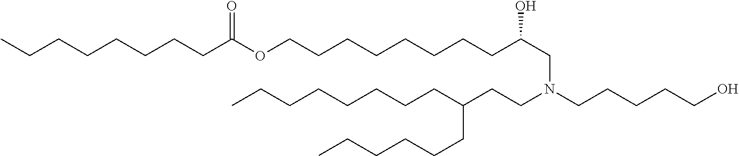 [SMILES: CCCCCCCCC(=O)OCCCCCCCC[C@H](O)CN(CCCCCO)CCC(CCCCCC)CCCCCCCC] |
| 53 | 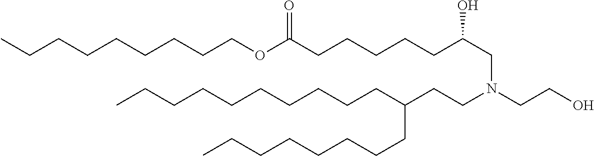 [SMILES: CCCCCCCCCCC(CCCCCCCC)CCN(CCO)C[C@@H](O)CCCCCC(=O)OCCCCCCCCC] |
| 54 | 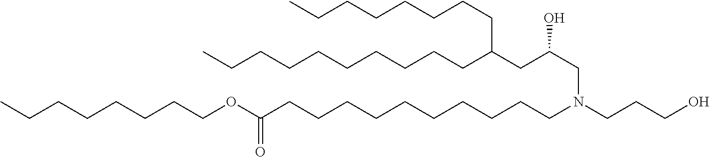 [SMILES: CCCCCCCCCCC(CCCCCCCC)C[C@H](O)CN(CCCO)CCCCCCCCCCC(=O)OCCCCCCCC] |
| 55 | 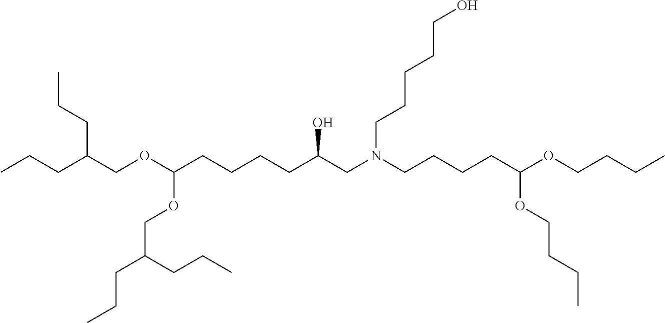 [SMILES: CCCCOC(CCCCN(CCCCCO)C[C@H](O)CCCCC(OCC(CCC)CCC)OCC(CCC)CCC)OCCCC] |
| 56 | 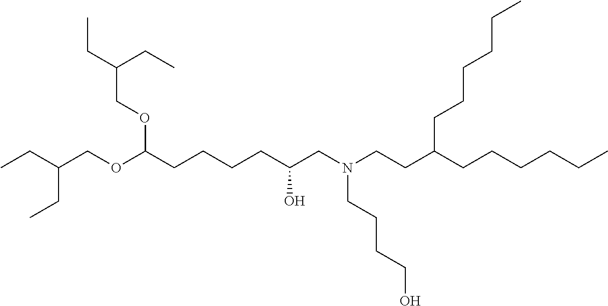 [SMILES: CCCCCCC(CCCCCC)CCN(CCCCO)C[C@H](O)CCCCC(OCC(CC)CC)OCC(CC)CC] |
| 57 | 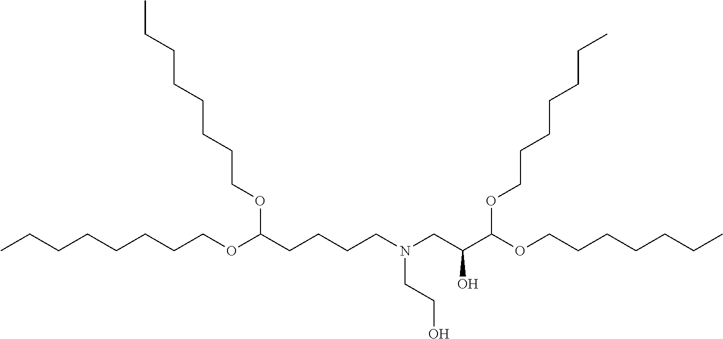 [SMILES: CCCCCCCCOC(CCCCN(CCO)C[C@H](O)C(OCCCCCCC)OCCCCCCC)OCCCCCCCC] |
| 58 | 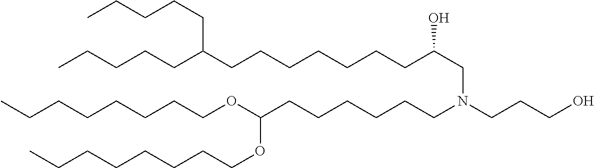 [SMILES: CCCCCCCCOC(CCCCCCN(CCCO)C[C@@H](O)CCCCCCCC(CCCCC)CCCCC)OCCCCCCCC] |
| 59 | 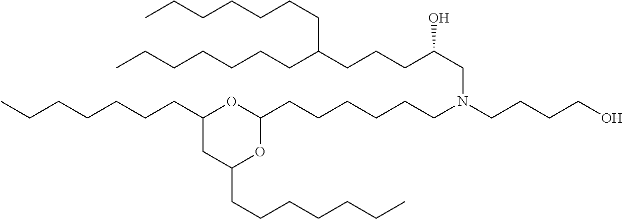 [SMILES: CCCCCCCC(CCCCCCC)CCC[C@H](O)CN(CCCCO)CCCCCCC1OC(CCCCCCC)CC(CCCCCCC)O1] |
| 60 | 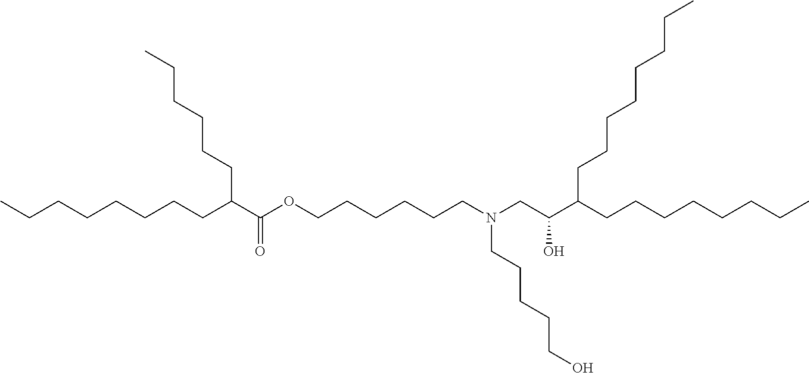 [SMILES: CCCCCCCCC(CCCCCC)C(=O)OCCCCCCN(CCCCCO)C[C@@H](O)C(CCCCCCCC)CCCCCCCC] |
| 61 | 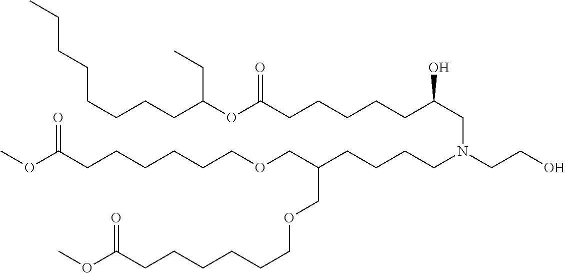 [SMILES: CCCCCCCCC(CC)OC(=O)CCCCC[C@@H](O)CN(CCO)CCCCC(COCCCCCCC(=O)OC)COCCCCCCC(=O)OC] |
| 62 | 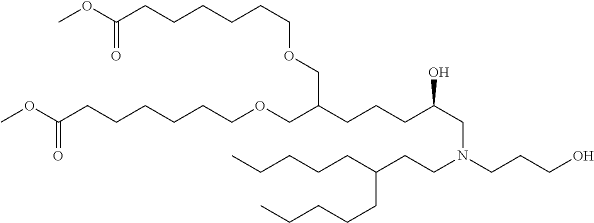 [SMILES: CCCCCC(CCCCC)CCN(CCCO)C[C@H](O)CCCC(COCCCCCCC(=O)OC)COCCCCCCC(=O)OC] |
| 63 | 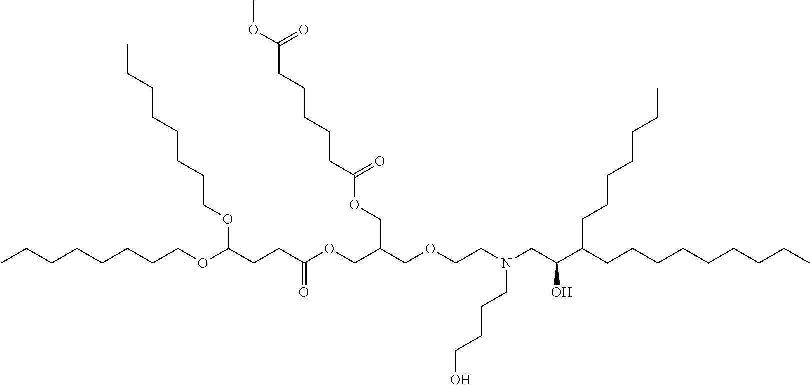 [SMILES: CCCCCCCCCC(CCCCCCC)[C@@H](O)CN(CCCCO)CCOCC(COC(=O)CCCCCC(=O)OC)COC(=O)CCC(OCCCCCCCC)OCCCCCCCC] |
| 64 | 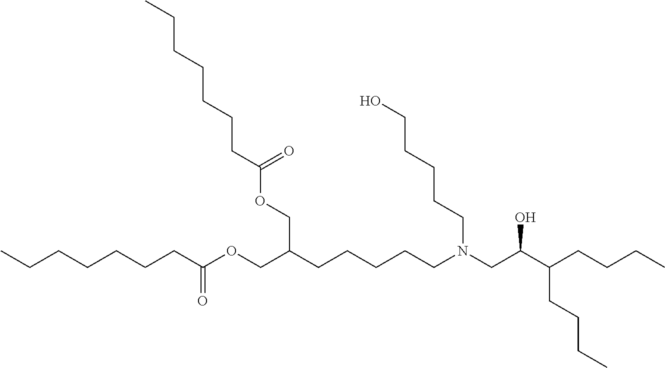 [SMILES: CCCCCCCC(=O)OCC(CCCCCN(CCCCCO)C[C@@H](O)C(CCCC)CCCC)COC(=O)CCCCCCC] |
| 65 | 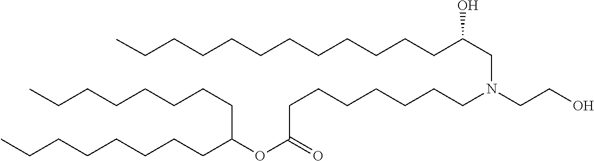 [SMILES: CCCCCCCCCCCC[C@H](O)CN(CCO)CCCCCCCC(=O)OC(CCCCCCCC)CCCCCCCC] |
| 66 | 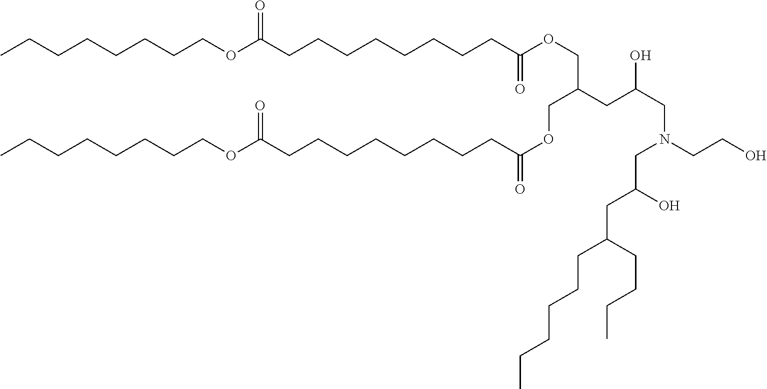 [SMILES: CCCCCCCCOC(=O)CCCCCCCCC(=O)OCC(COC(=O)CCCCCCCCC(=O)OCCCCCCCC)CC(O)CN(CCO)CC(O)CC(CCCC)CCCCCC] |
| 67 | 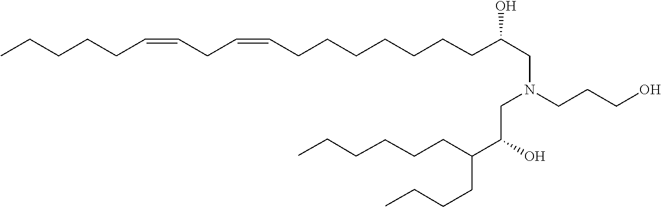 [SMILES: CCCCC/C=C\C/C=C\CCCCCCC[C@H](O)CN(CCCO)C[C@H](O)C(CCCC)CCCCCC] |
| 68 | 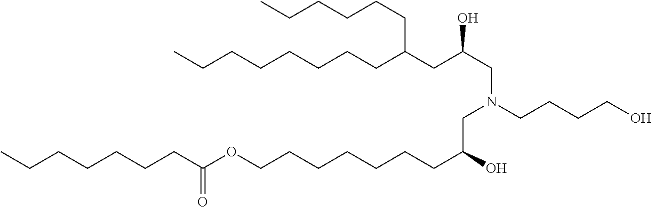 [SMILES: CCCCCCCCC(CCCCCC)C[C@@H](O)CN(CCCCO)C[C@@H](O)CCCCCCCOC(=O)CCCCCCC] |
| 69 | 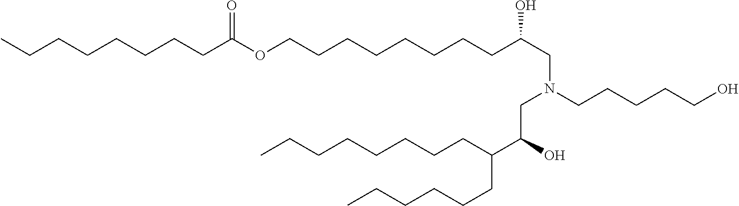 [SMILES: CCCCCCCCC(=O)OCCCCCCCC[C@H](O)CN(CCCCCO)C[C@@H](O)C(CCCCCC)CCCCCCCC] |
| 70 | 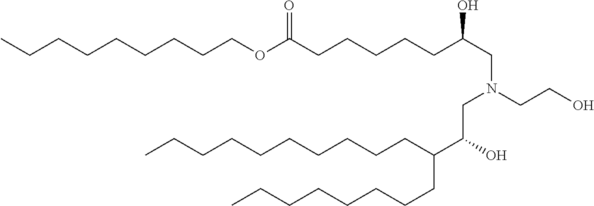 [SMILES: CCCCCCCCCCC(CCCCCCCC)[C@@H](O)CN(CCO)C[C@H](O)CCCCCC(=O)OCCCCCCCCC] |
| 71 | 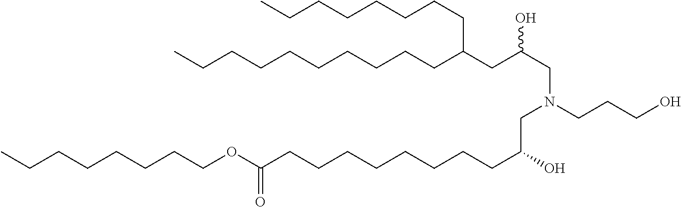 [SMILES: CCCCCCCCCCC(CCCCCCCC)CC(O)CN(CCCO)C[C@H](O)CCCCCCCCC(=O)OCCCCCCCC] |
| 72 | 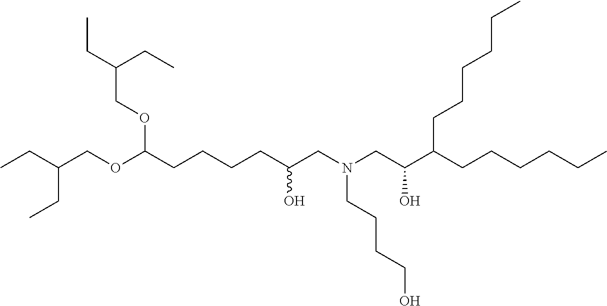 [SMILES: CCCCCCC(CCCCCC)[C@H](O)CN(CCCCO)CC(O)CCCCC(OCC(CC)CC)OCC(CC)CC] |
| 73 | 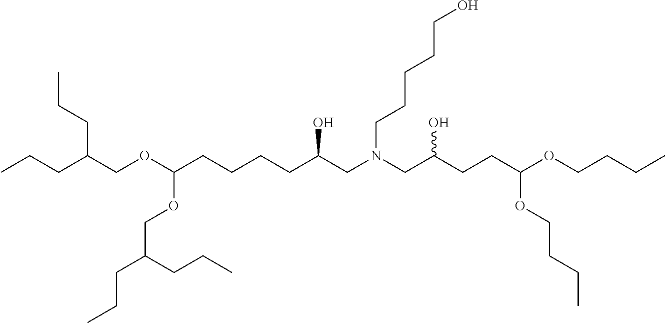 [SMILES: CCCCOC(CCC(O)CN(CCCCCO)C[C@H](O)CCCCC(OCC(CCC)CCC)OCC(CCC)CCC)OCCCC] |
| 74 | 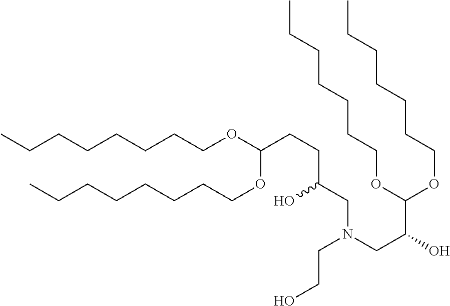 [SMILES: CCCCCCCCOC(CCC(O)CN(CCO)C[C@@H](O)C(OCCCCCCC)OCCCCCCC)OCCCCCCCC] |
| 75 | 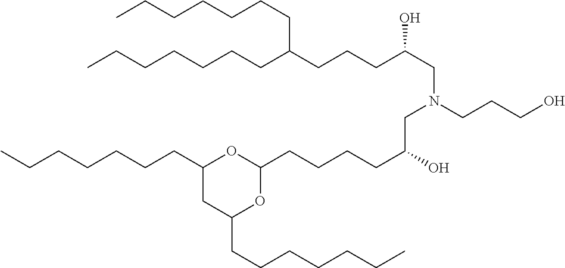 [SMILES: CCCCCCCC(CCCCCCC)CCC[C@H](O)CN(CCCO)C[C@H](O)CCCCC1OC(CCCCCCC)CC(CCCCCCC)O1] |
| 76 | 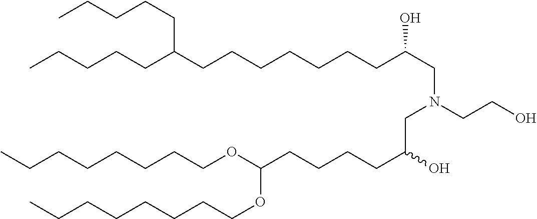 [SMILES: CCCCCCCCOC(CCCCC(O)CN(CCO)C[C@@H](O)CCCCCCCC(CCCCC)CCCCC)OCCCCCCCC] |
| 77 | 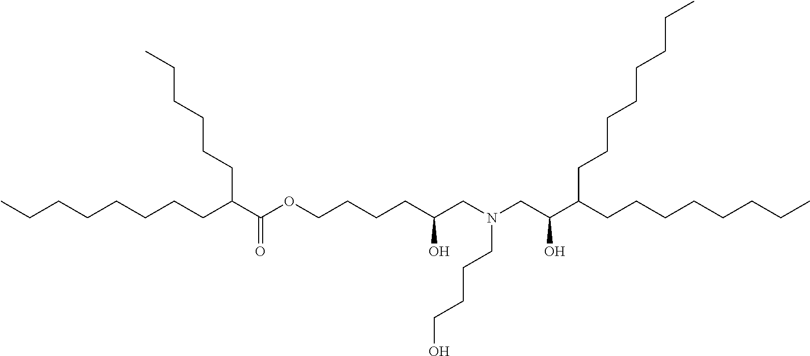 [SMILES: CCCCCCCCC(CCCCCC)C(=O)OCCCC[C@H](O)CN(CCCCO)C[C@H](O)C(CCCCCCCC)CCCCCCCC] |
| 78 | 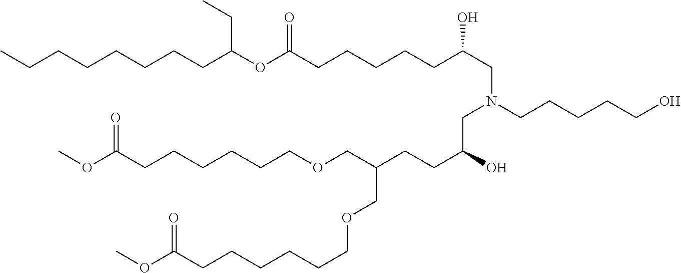 [SMILES: CCCCCCCCC(CC)OC(=O)CCCCC[C@H](O)CN(CCCCCO)C[C@@H](O)CCC(COCCCCCCC(=O)OC)COCCCCCCC(=O)OC] |
| 79 | 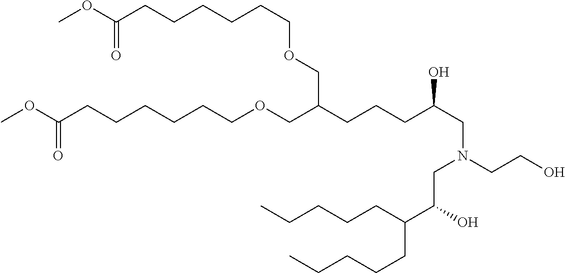 [SMILES: CCCCCC(CCCCC)[C@@H](O)CN(CCO)C[C@H](O)CCCC(COCCCCCCC(=O)OC)COCCCCCCC(=O)OC] |
| 80 | 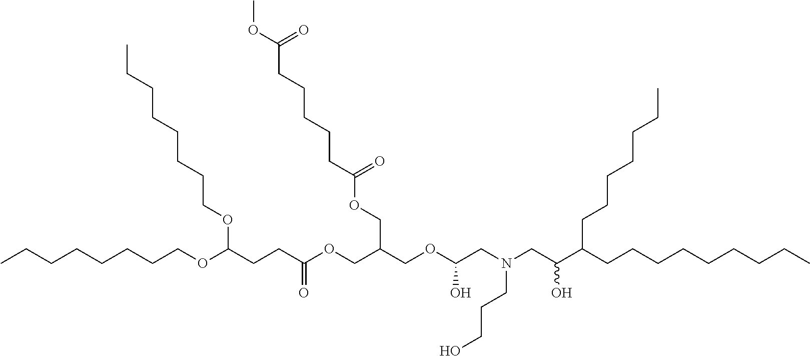 [SMILES: CCCCCCCCCC(CCCCCCC)C(O)CN(CCCO)C[C@H](O)OCC(COC(=O)CCCCCC(=O)OC)COC(=O)CCC(OCCCCCCCC)OCCCCCCCC] |
| 81 | 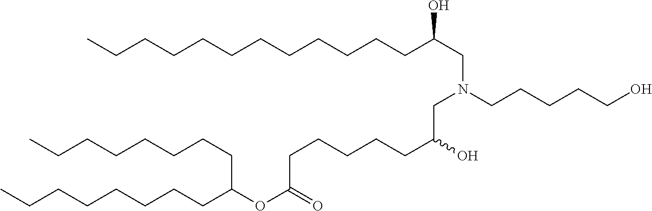 [SMILES: CCCCCCCCCCCC[C@@H](O)CN(CCCCCO)CC(O)CCCCCC(=O)OC(CCCCCCCC)CCCCCCCC] |
| 82 | 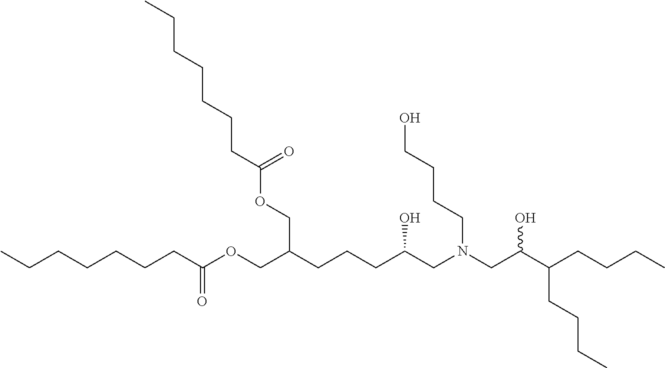 [SMILES: CCCCCCCC(=O)OCC(CCC[C@H](O)CN(CCCCO)CC(O)C(CCCC)CCCC)COC(=O)CCCCCCC] |
| 83 | 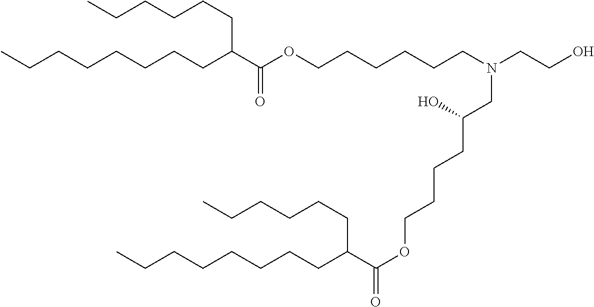 [SMILES: CCCCCCCCC(CCCCCC)C(=O)OCCCCCCN(CCO)C[C@@H](O)CCCCOC(=O)C(CCCCCC)CCCCCCCC] |
| 84 | 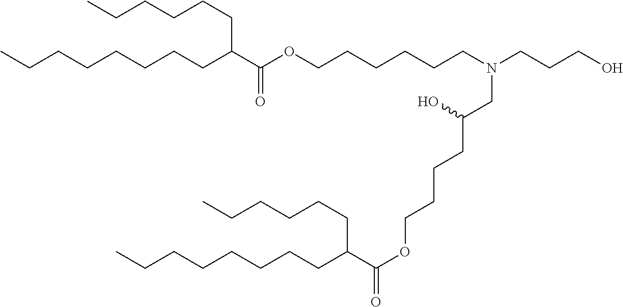 [SMILES: CCCCCCCCC(CCCCCC)C(=O)OCCCCCCN(CCCO)CC(O)CCCCOC(=O)C(CCCCCC)CCCCCCCC] |
| 85 | 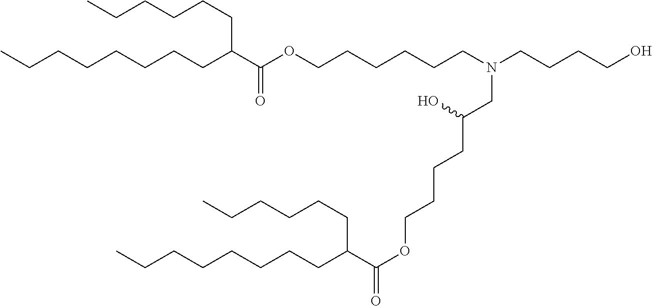 [SMILES: CCCCCCCCC(CCCCCC)C(=O)OCCCCCCN(CCCCO)CC(O)CCCCOC(=O)C(CCCCCC)CCCCCCCC] |
| 86 | 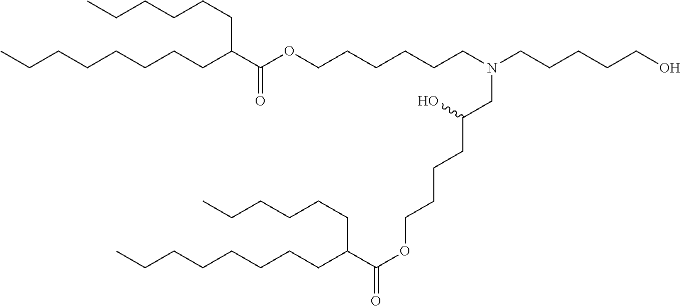 [SMILES: CCCCCCCCC(CCCCCC)C(=O)OCCCCCCN(CCCCCO)CC(O)CCCCOC(=O)C(CCCCCC)CCCCCCCC] |
| 87 | 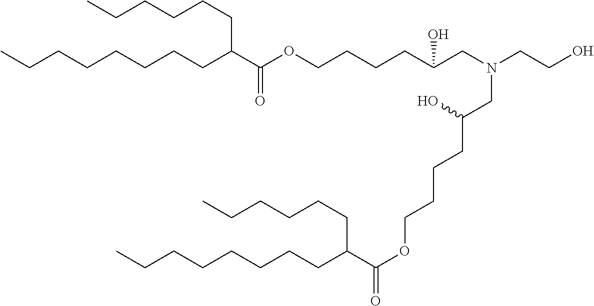 [SMILES: CCCCCCCCC(CCCCCC)C(=O)OCCCCC(O)CN(CCO)C[C@H](O)CCCCOC(=O)C(CCCCCC)CCCCCCCC] |
| 88 | 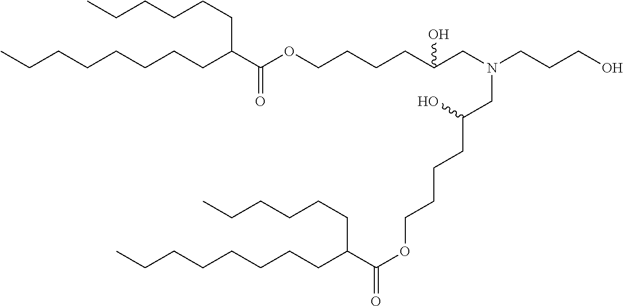 [SMILES: CCCCCCCCC(CCCCCC)C(=O)OCCCCC(O)CN(CCCO)CC(O)CCCCOC(=O)C(CCCCCC)CCCCCCCC] |
| 89 | 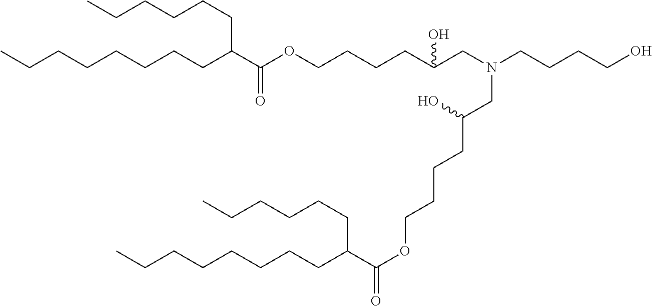 [SMILES: CCCCCCCCC(CCCCCC)C(=O)OCCCCC(O)CN(CCCCO)CC(O)CCCCOC(=O)C(CCCCCC)CCCCCCCC] |
| 90 | 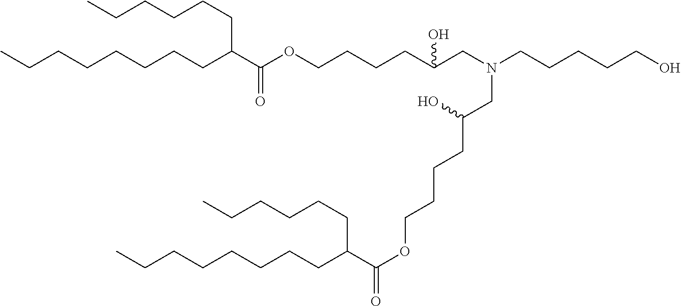 [SMILES: CCCCCCCCC(CCCCCC)C(=O)OCCCCC(O)CN(CCCCCO)CC(O)CCCCOC(=O)C(CCCCCC)CCCCCCCC] |
| 91 | 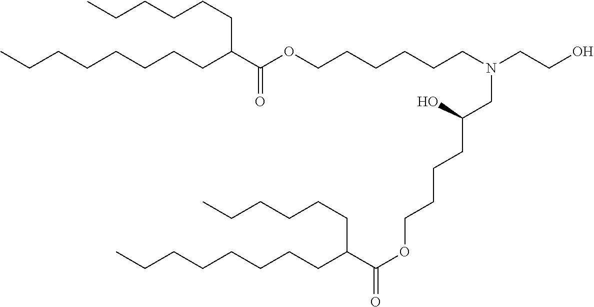 [SMILES: CCCCCCCCC(CCCCCC)C(=O)OCCCCCCN(CCO)C[C@H](O)CCCCOC(=O)C(CCCCCC)CCCCCCCC] |
| 92 | 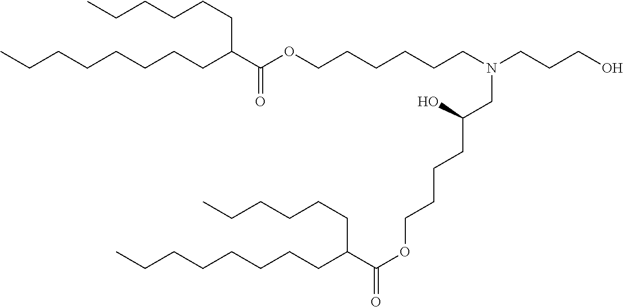 [SMILES: CCCCCCCCC(CCCCCC)C(=O)OCCCCCCN(CCCO)C[C@H](O)CCCCOC(=O)C(CCCCCC)CCCCCCCC] |
| 93 | 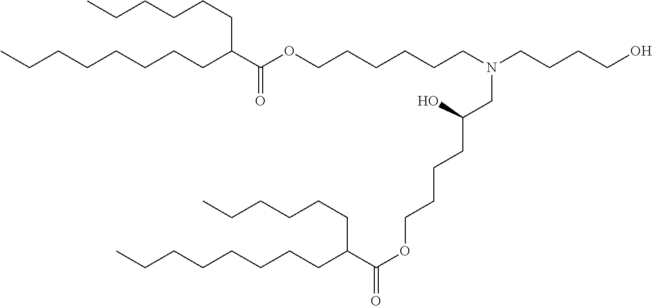 [SMILES: CCCCCCCCC(CCCCCC)C(=O)OCCCCCCN(CCCCO)C[C@H](O)CCCCOC(=O)C(CCCCCC)CCCCCCCC] |
| 94 | 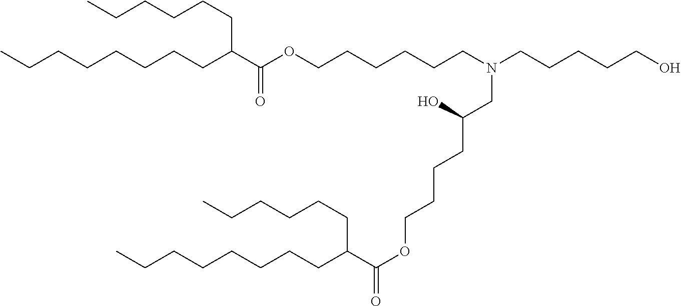 [SMILES: CCCCCCCCC(CCCCCC)C(=O)OCCCCCCN(CCCCCO)C[C@H](O)CCCCOC(=O)C(CCCCCC)CCCCCCCC] |
| 95 | 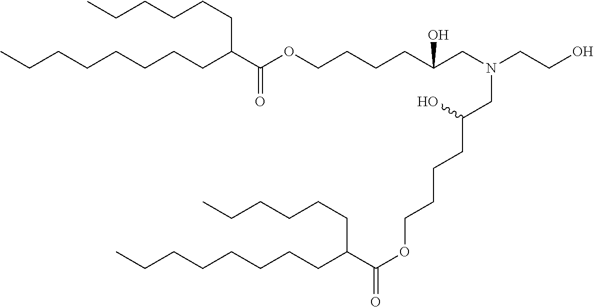 [SMILES: CCCCCCCCC(CCCCCC)C(=O)OCCCCC(O)CN(CCO)C[C@@H](O)CCCCOC(=O)C(CCCCCC)CCCCCCCC] |
| 96 | 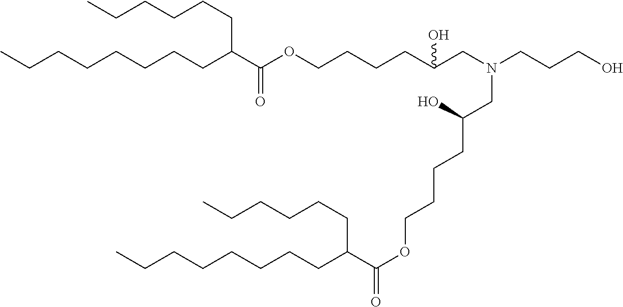 [SMILES: CCCCCCCCC(CCCCCC)C(=O)OCCCCC(O)CN(CCCO)C[C@H](O)CCCCOC(=O)C(CCCCCC)CCCCCCCC] |
| 97 | 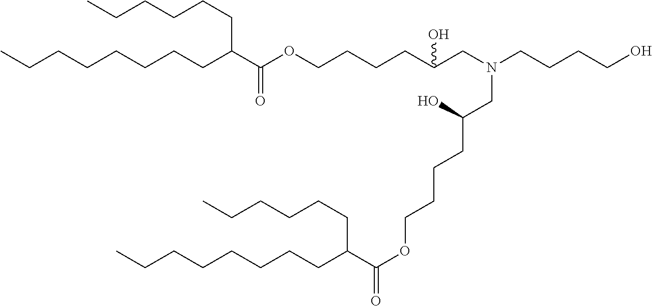 [SMILES: CCCCCCCCC(CCCCCC)C(=O)OCCCCC(O)CN(CCCCO)C[C@H](O)CCCCOC(=O)C(CCCCCC)CCCCCCCC] |
| 98 | 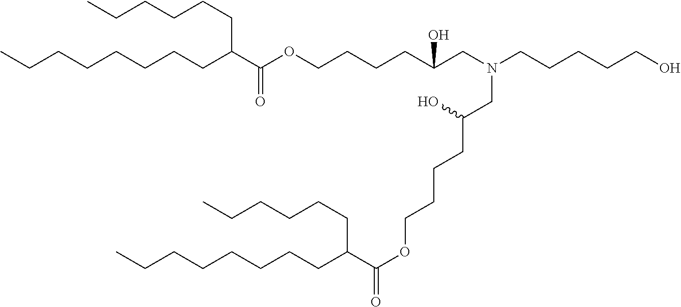 [SMILES: CCCCCCCCC(CCCCCC)C(=O)OCCCCC(O)CN(CCCCCO)C[C@@H](O)CCCCOC(=O)C(CCCCCC)CCCCCCCC] |
| 99 | 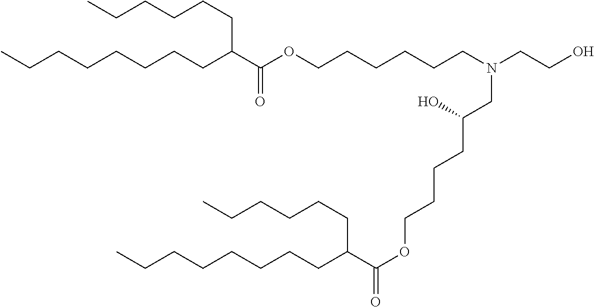 [SMILES: CCCCCCCCC(CCCCCC)C(=O)OCCCCCCN(CCO)C[C@@H](O)CCCCOC(=O)C(CCCCCC)CCCCCCCC] |
| 100 | 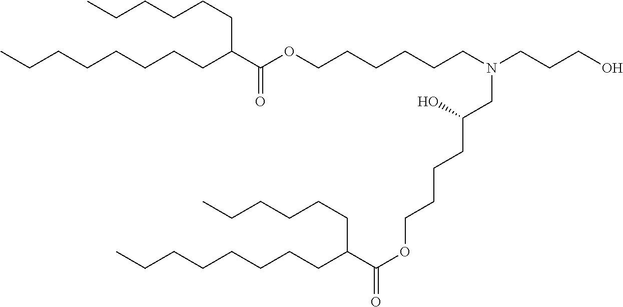 [SMILES: CCCCCCCCC(CCCCCC)C(=O)OCCCCCCN(CCCO)C[C@@H](O)CCCCOC(=O)C(CCCCCC)CCCCCCCC] |
| 101 | 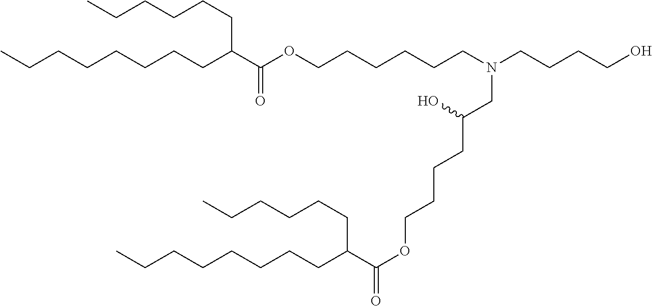 [SMILES: CCCCCCCCC(CCCCCC)C(=O)OCCCCCCN(CCCCO)CC(O)CCCCOC(=O)C(CCCCCC)CCCCCCCC] |
| 102 | 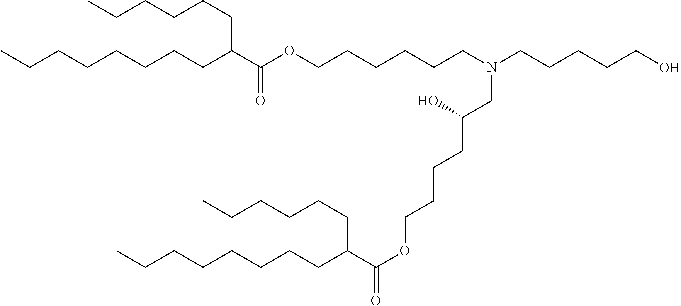 [SMILES: CCCCCCCCC(CCCCCC)C(=O)OCCCCCCN(CCCCCO)C[C@@H](O)CCCCOC(=O)C(CCCCCC)CCCCCCCC] |
| 103 | 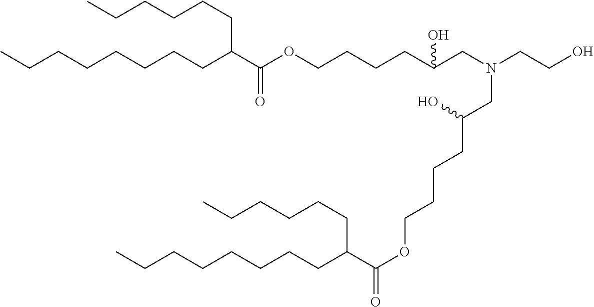 [SMILES: CCCCCCCCC(CCCCCC)C(=O)OCCCCC(O)CN(CCO)CC(O)CCCCOC(=O)C(CCCCCC)CCCCCCCC] |
| 104 | 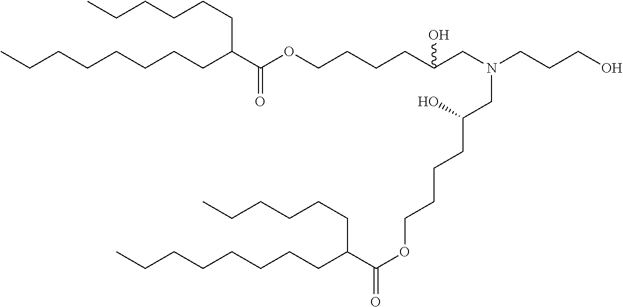 [SMILES: CCCCCCCCC(CCCCCC)C(=O)OCCCCC(O)CN(CCCO)C[C@@H](O)CCCCOC(=O)C(CCCCCC)CCCCCCCC] |
| 105 | 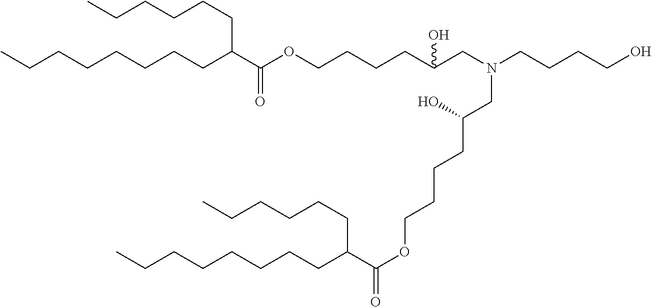 [SMILES: CCCCCCCCC(CCCCCC)C(=O)OCCCCC(O)CN(CCCCO)C[C@@H](O)CCCCOC(=O)C(CCCCCC)CCCCCCCC] |
| 106 | 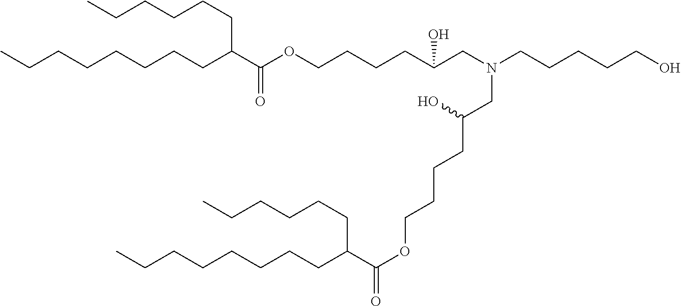 [SMILES: CCCCCCCCC(CCCCCC)C(=O)OCCCCC(O)CN(CCCCCO)C[C@H](O)CCCCOC(=O)C(CCCCCC)CCCCCCCC] |
| 107 | 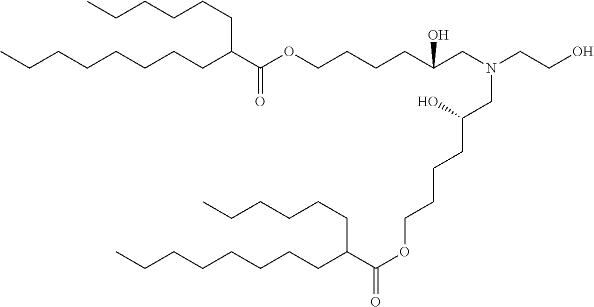 [SMILES: CCCCCCCCC(CCCCCC)C(=O)OCCCC[C@H](O)CN(CCO)C[C@@H](O)CCCCOC(=O)C(CCCCCC)CCCCCCCC] |
| 108 | 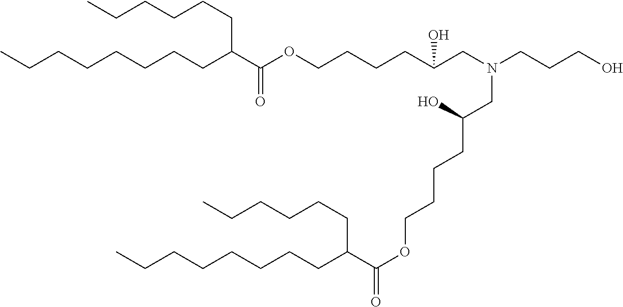 [SMILES: CCCCCCCCC(CCCCCC)C(=O)OCCCC[C@@H](O)CN(CCCO)C[C@H](O)CCCCOC(=O)C(CCCCCC)CCCCCCCC] |
| 109 | 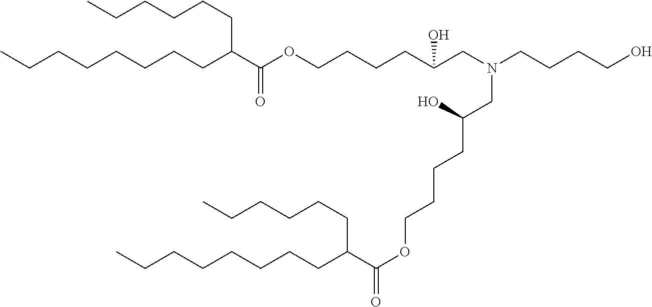 [SMILES: CCCCCCCCC(CCCCCC)C(=O)OCCCC[C@@H](O)CN(CCCCO)C[C@H](O)CCCCOC(=O)C(CCCCCC)CCCCCCCC] |
| 110 | 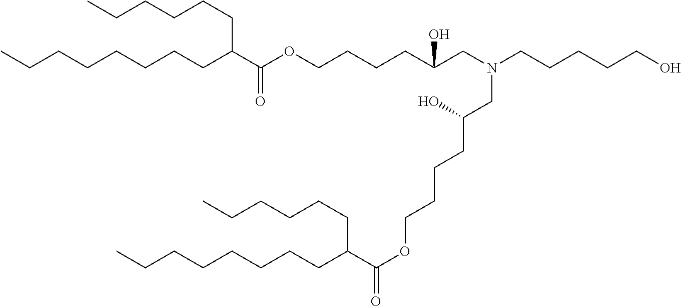 [SMILES: CCCCCCCCC(CCCCCC)C(=O)OCCCC[C@H](O)CN(CCCCCO)C[C@@H](O)CCCCOC(=O)C(CCCCCC)CCCCCCCC] |
| 111 | 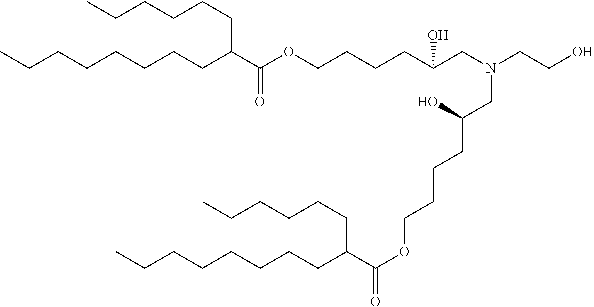 [SMILES: CCCCCCCCC(CCCCCC)C(=O)OCCCC[C@@H](O)CN(CCO)C[C@H](O)CCCCOC(=O)C(CCCCCC)CCCCCCCC] |
| 112 | 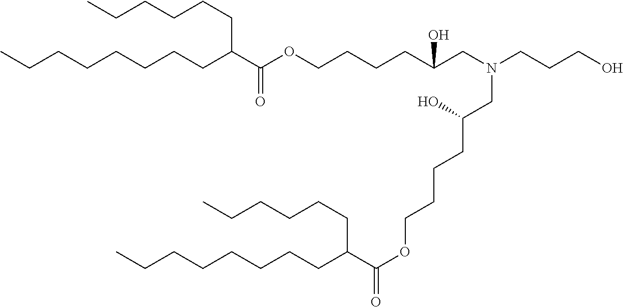 [SMILES: CCCCCCCCC(CCCCCC)C(=O)OCCCC[C@H](O)CN(CCCO)C[C@@H](O)CCCCOC(=O)C(CCCCCC)CCCCCCCC] |
| 113 | 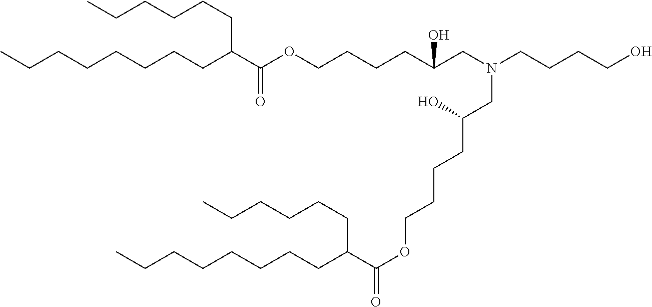 [SMILES: CCCCCCCCC(CCCCCC)C(=O)OCCCC[C@H](O)CN(CCCCO)C[C@@H](O)CCCCOC(=O)C(CCCCCC)CCCCCCCC] |
| 114 | 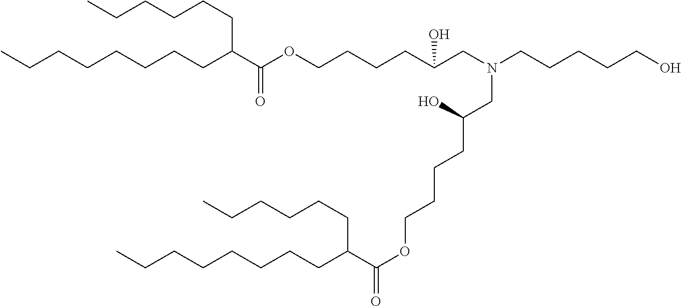 [SMILES: CCCCCCCCC(CCCCCC)C(=O)OCCCC[C@@H](O)CN(CCCCCO)C[C@H](O)CCCCOC(=O)C(CCCCCC)CCCCCCCC] |
| 115 | 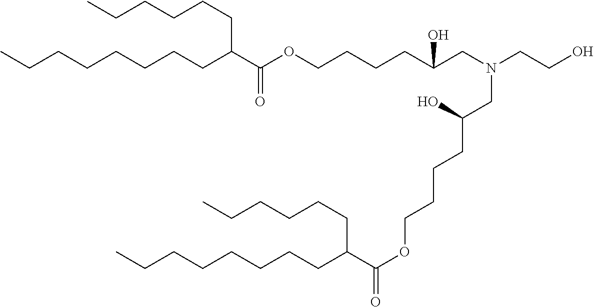 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 116 | 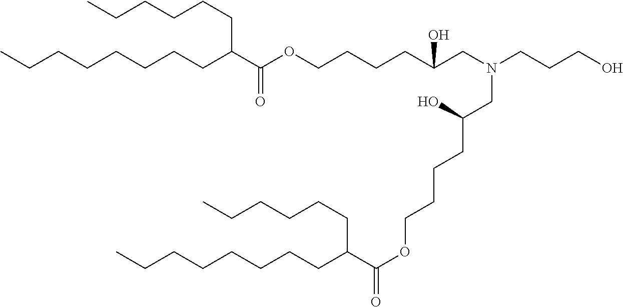 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 117 | 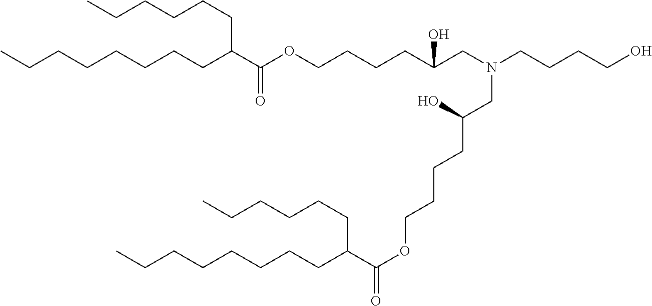 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 118 | 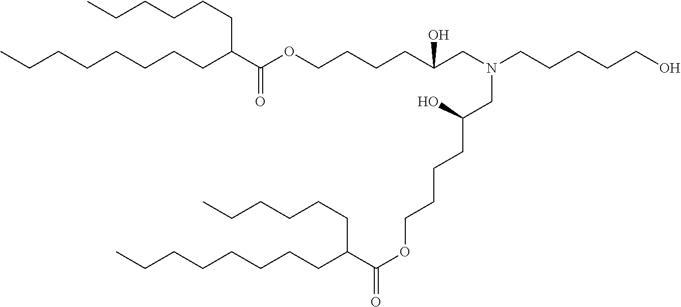 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 119 | 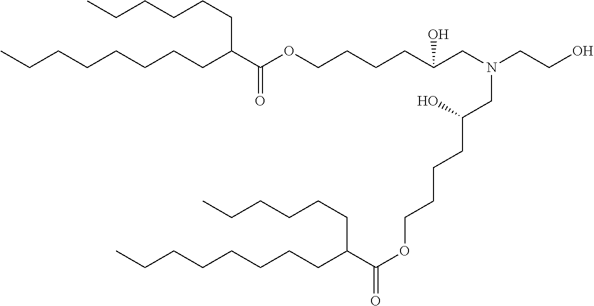 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 120 | 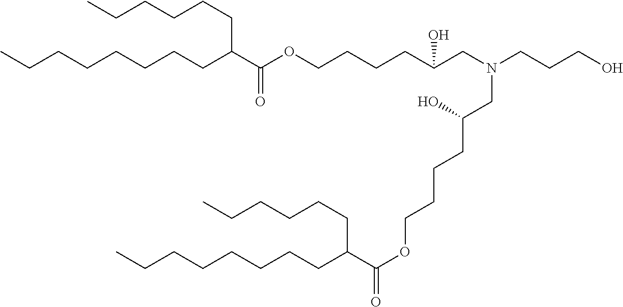 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 121 | 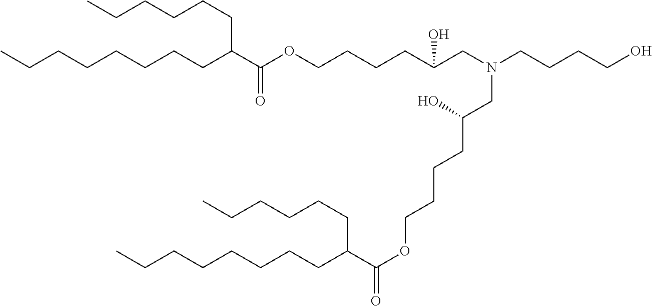 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 122 | 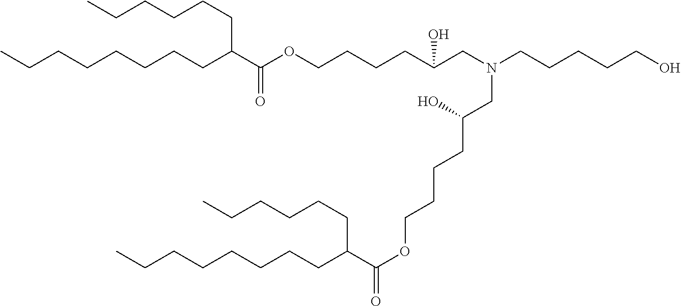 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 123 | 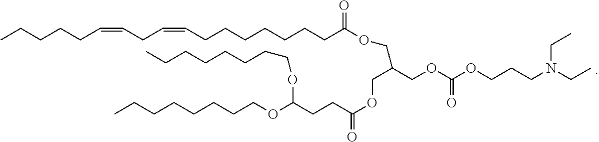 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 124 | 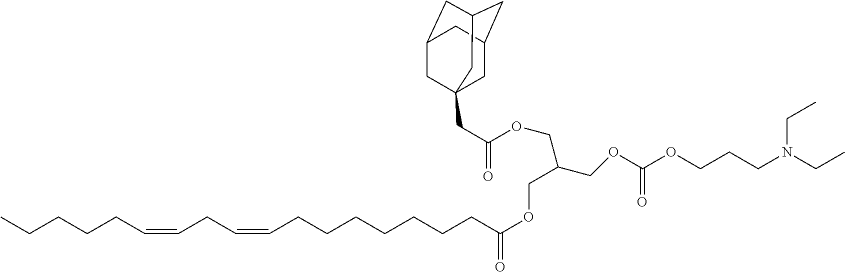 |
| 125 | 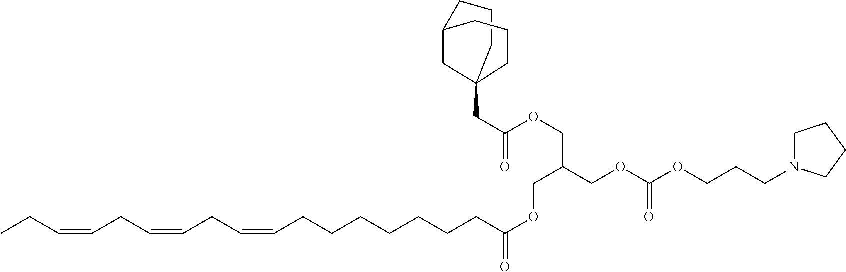 |
| 126 | 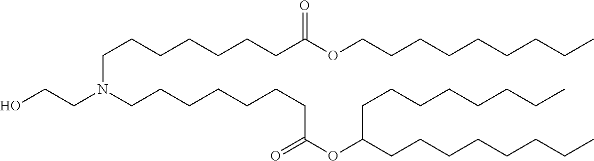 [SMILES: CCCCCCCCCOC(=O)CCCCCCCN(CCO)CCCCCCCC(=O)OC(CCCCCCCC)CCCCCCCC] |
| 127 | 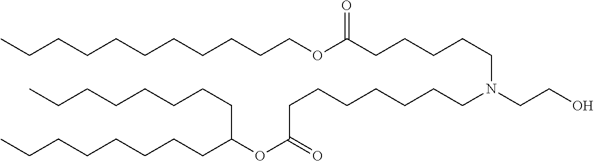 [SMILES: CCCCCCCCCCCOC(=O)CCCCCN(CCO)CCCCCCCC(=O)OC(CCCCCCCC)CCCCCCCC] |
| 128 | 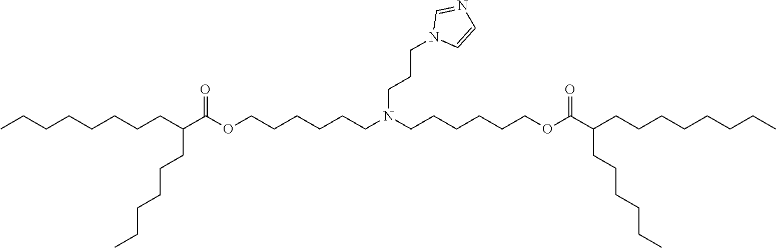 [SMILES: CCCCCCCCC(CCCCCC)C(=O)OCCCCCCN(CCCCCCOC(=O)C(CCCCCC)CCCCCCCC)CCCn1ccnc1] |
| 129 | 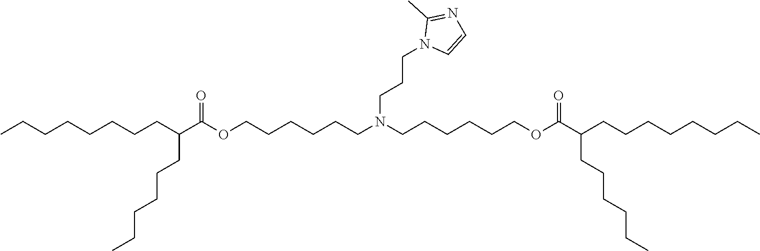 [SMILES: CCCCCCCCC(CCCCCC)C(=O)OCCCCCCN(CCCCCCOC(=O)C(CCCCCC)CCCCCCCC)CCCn1ccnc1C] |
| 130 | 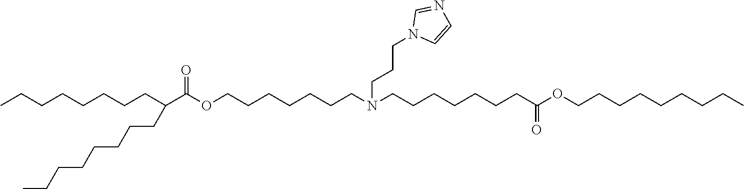 [SMILES: CCCCCCCCCOC(=O)CCCCCCCN(CCCCCCCOC(=O)C(CCCCCCCC)CCCCCCCC)CCCn1ccnc1] |
| 131 | 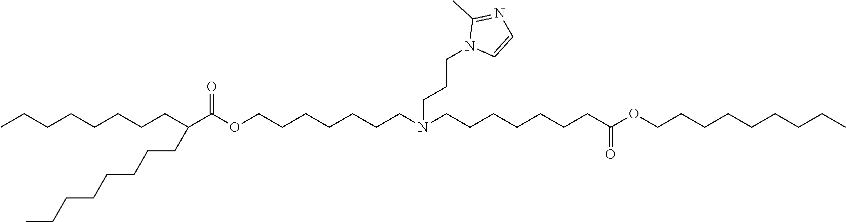 [SMILES: CCCCCCCCCOC(=O)CCCCCCCN(CCCCCCCOC(=O)C(CCCCCCCC)CCCCCCCC)CCCn1ccnc1C] |
| 132 | 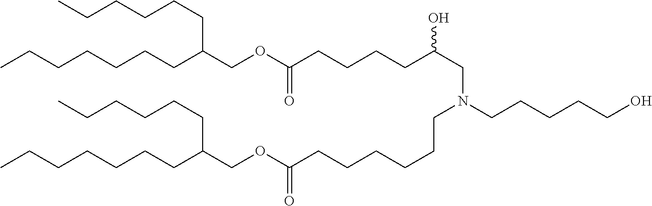 [SMILES: CCCCCCCC(CCCCCC)COC(=O)CCCCCCN(CCCCCO)CC(O)CCCCC(=O)OCC(CCCCCC)CCCCCCC] |
| 133 | 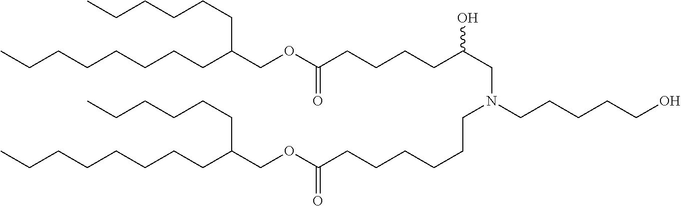 [SMILES: CCCCCCCCC(CCCCCC)COC(=O)CCCCCCN(CCCCCO)CC(O)CCCCC(=O)OCC(CCCCCC)CCCCCCCC] |
| 134 | 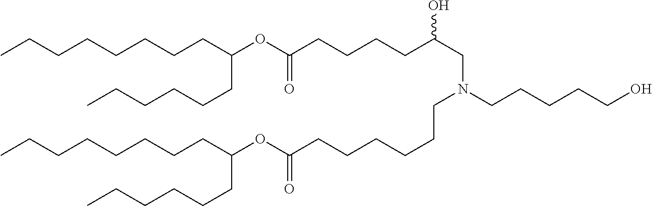 [SMILES: CCCCCCCCC(CCCCCC)OC(=O)CCCCCCN(CCCCCO)CC(O)CCCCC(=O)OC(CCCCCC)CCCCCCCC] |
| 135 | 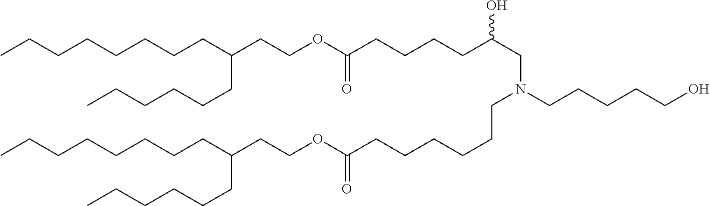 [SMILES: CCCCCCCCC(CCCCCC)CCOC(=O)CCCCCCN(CCCCCO)CC(O)CCCCC(=O)OCCC(CCCCCC)CCCCCCCC] |
| 136 | 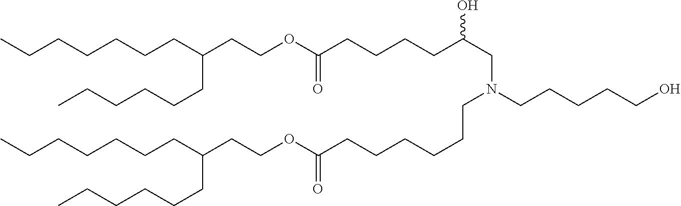 [SMILES: CCCCCCCC(CCCCCC)CCOC(=O)CCCCCCN(CCCCCO)CC(O)CCCCC(=O)OCCC(CCCCCC)CCCCCCC] |
| 137 | 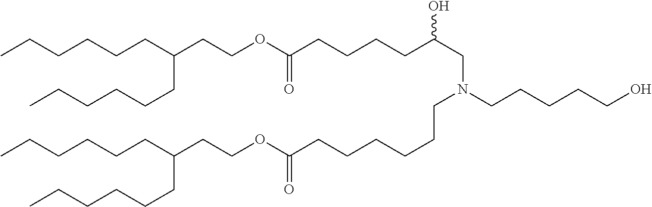 [SMILES: CCCCCCC(CCCCCC)CCOC(=O)CCCCCCN(CCCCCO)CC(O)CCCCC(=O)OCCC(CCCCCC)CCCCCC] |
| 138 | 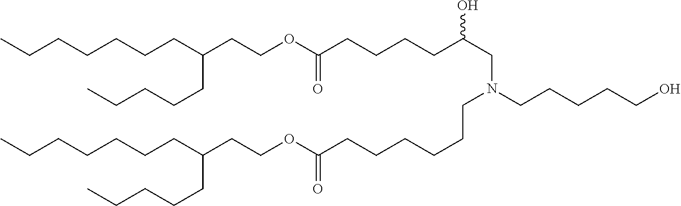 [SMILES: CCCCCCCC(CCCCC)CCOC(=O)CCCCCCN(CCCCCO)CC(O)CCCCC(=O)OCCC(CCCCC)CCCCCCC] |
| 139 | 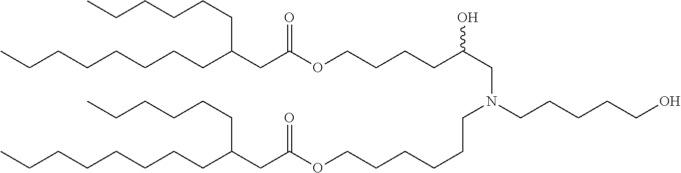 [SMILES: CCCCCCCCC(CCCCCC)CC(=O)OCCCCCCN(CCCCCO)CC(O)CCCCOC(=O)CC(CCCCCC)CCCCCCCC] |
| 140 | 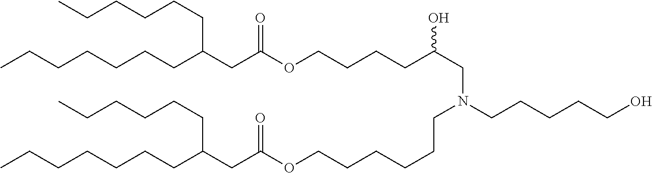 [SMILES: CCCCCCCC(CCCCCC)CC(=O)OCCCCCCN(CCCCCO)CC(O)CCCCOC(=O)CC(CCCCCC)CCCCCCC] |
| 141 | 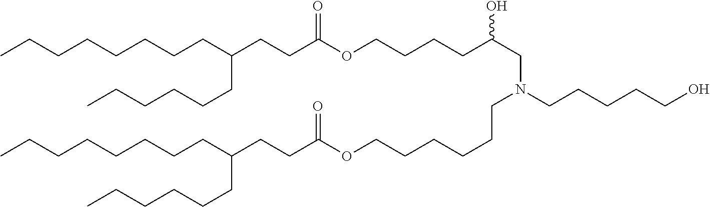 [SMILES: CCCCCCCCC(CCCCCC)CCC(=O)OCCCCCCN(CCCCCO)CC(O)CCCCOC(=O)CCC(CCCCCC)CCCCCCCC] |
| 142 | 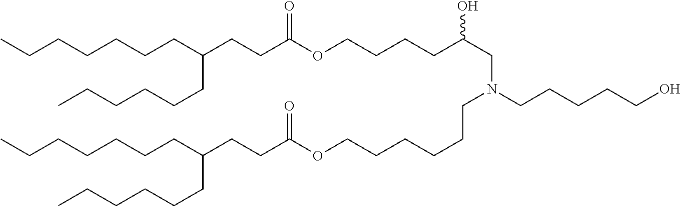 [SMILES: CCCCCCCC(CCCCCC)CCC(=O)OCCCCCCN(CCCCCO)CC(O)CCCCOC(=O)CCC(CCCCCC)CCCCCCC] |
| 143 | 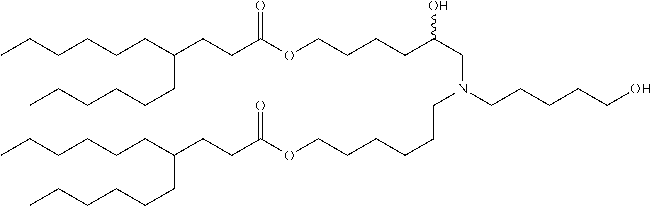 [SMILES: CCCCCCC(CCCCCC)CCC(=O)OCCCCCCN(CCCCCO)CC(O)CCCCOC(=O)CCC(CCCCCC)CCCCCC] |
| 144 | 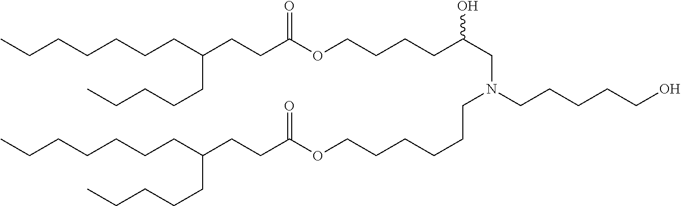 [SMILES: CCCCCCCC(CCCCC)CCC(=O)OCCCCCCN(CCCCCO)CC(O)CCCCOC(=O)CCC(CCCCC)CCCCCCC] |
| 145 | 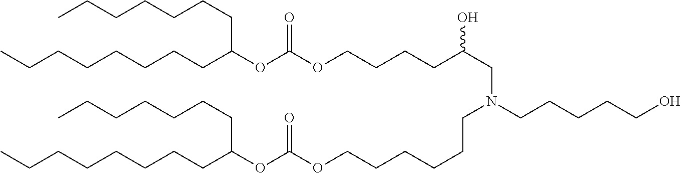 [SMILES: CCCCCCCCC(CCCCCCC)OC(=O)OCCCCCCN(CCCCCO)CC(O)CCCCOC(=O)OC(CCCCCCC)CCCCCCCC] |
| 146 | 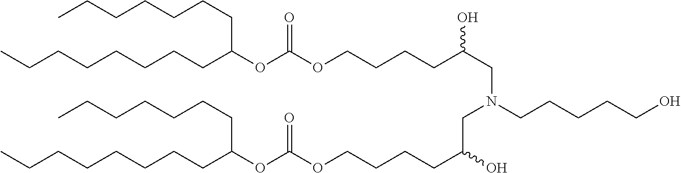 [SMILES: CCCCCCCCC(CCCCCCC)OC(=O)OCCCCC(O)CN(CCCCCO)CC(O)CCCCOC(=O)OC(CCCCCCC)CCCCCCCC] |
| 147 | 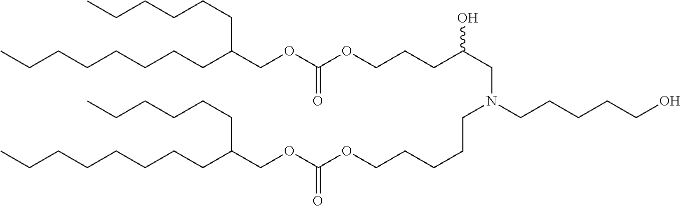 [SMILES: CCCCCCCCC(CCCCCC)COC(=O)OCCCCCN(CCCCCO)CC(O)CCCOC(=O)OCC(CCCCCC)CCCCCCCC] |
| 148 | 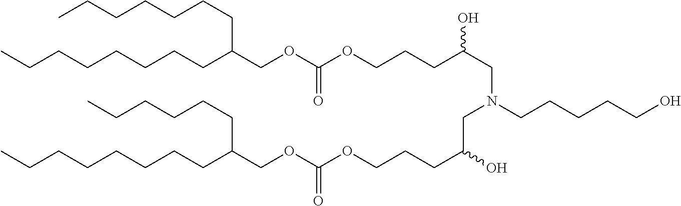 [SMILES: CCCCCCCCC(CCCCCC)COC(=O)OCCCC(O)CN(CCCCCO)CC(O)CCCOC(=O)OCC(CCCCCCC)CCCCCCCC] |
| 149 | 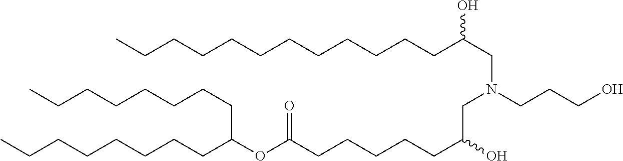 [SMILES: CCCCCCCCCCCCC(O)CN(CCCO)CC(O)CCCCCC(=O)OC(CCCCCCCC)CCCCCCCC] |
| 150 | 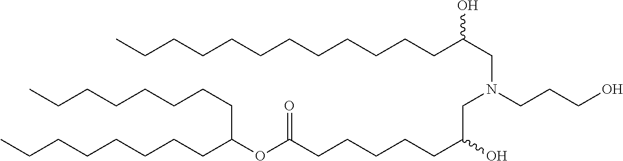 [SMILES: CCCCCCCCCCCCC(O)CN(CCCO)CC(O)CCCCCC(=O)OC(CCCCCCCC)CCCCCCCC] |
| 151 | 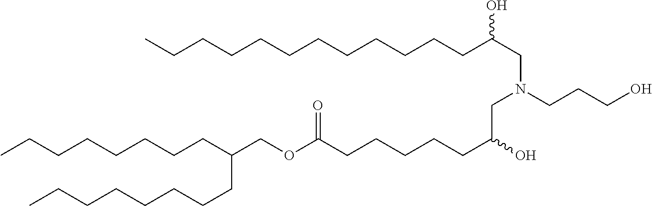 [SMILES: CCCCCCCCCCCCC(O)CN(CCCO)CC(O)CCCCCC(=O)OCC(CCCCCCCC)CCCCCCCC] |
| 152 | 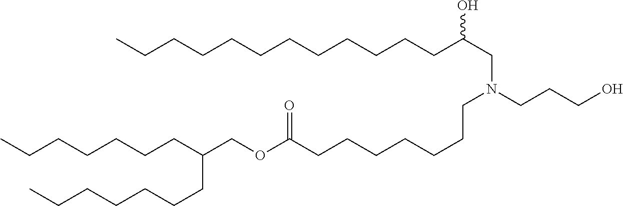 [SMILES: CCCCCCCCCCCCC(O)CN(CCCO)CCCCCCCC(=O)OCC(CCCCCCC)CCCCCCC] |
| 153 | 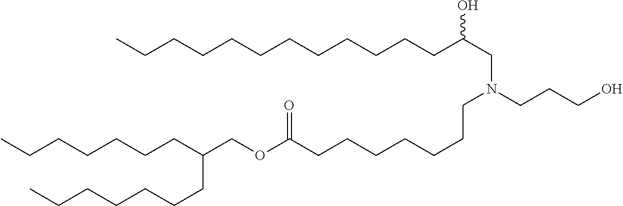 [SMILES: CCCCCCCCCCCCC(O)CN(CCCO)CCCCCCCC(=O)OCC(CCCCCCC)CCCCCCC] |
| 154 | 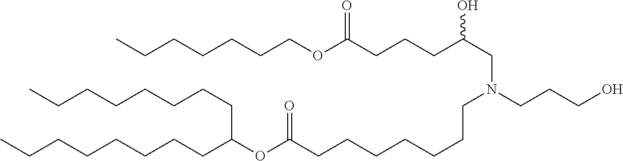 [SMILES: CCCCCCCCC(CCCCCCCC)OC(=O)CCCCCCCN(CCCO)CC(O)CCCC(=O)OCCCCCCC] |
| 155 | 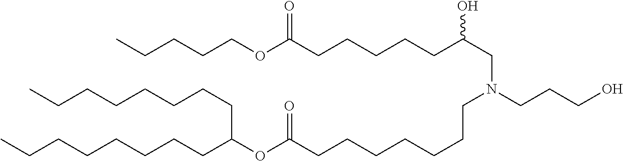 [SMILES: CCCCCCCCC(CCCCCCCC)OC(=O)CCCCCCCN(CCCO)CC(O)CCCCCC(=O)OCCCCC] |
| 156 | 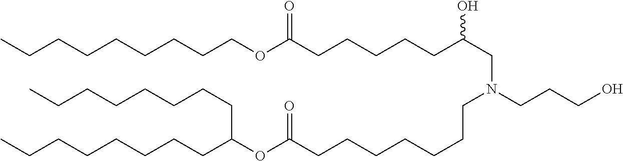 [SMILES: CCCCCCCCCOC(=O)CCCCCC(O)CN(CCCO)CCCCCCCC(=O)OC(CCCCCCCC)CCCCCCCC] |
| 157 | 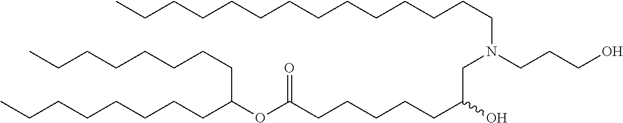 [SMILES: CCCCCCCCCCCCCCN(CCCO)CC(O)CCCCCC(=O)OC(CCCCCCCC)CCCCCCCC] |
| 158 | 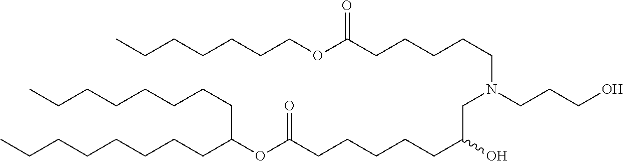 [SMILES: CCCCCCCCC(CCCCCCCC)OC(=O)CCCCCC(O)CN(CCCO)CCCCCC(=O)OCCCCCCC] |
| 159 | 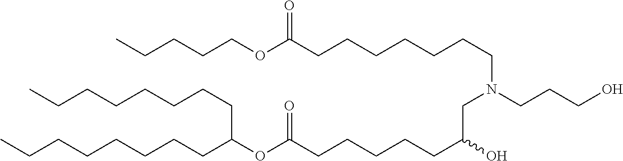 [SMILES: CCCCCCCCC(CCCCCCCC)OC(=O)CCCCCC(O)CN(CCCO)CCCCCCCC(=O)OCCCCC] |
| 160 | 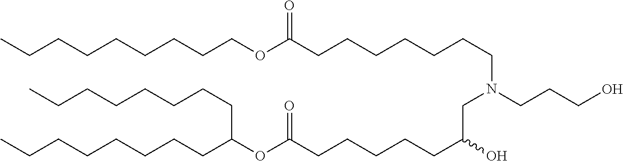 [SMILES: CCCCCCCCCOC(=O)CCCCCCCN(CCCO)CC(O)CCCCCC(=O)OC(CCCCCCCC)CCCCCCCC] |
| 161 | 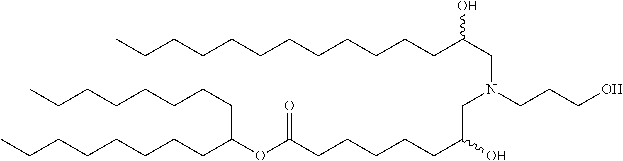 [SMILES: CCCCCCCCCCCCC(O)CN(CCCO)CC(O)CCCCCC(=O)OC(CCCCCCCC)CCCCCCCC] |
| 162 | 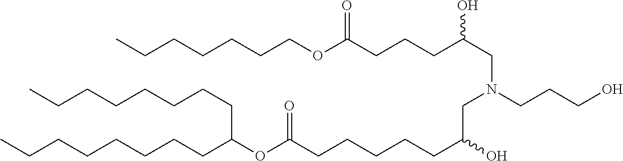 [SMILES: CCCCCCCCC(CCCCCCCC)OC(=O)CCCCCC(O)CN(CCCO)CC(O)CCCC(=O)OCCCCCCC] |
| 163 | 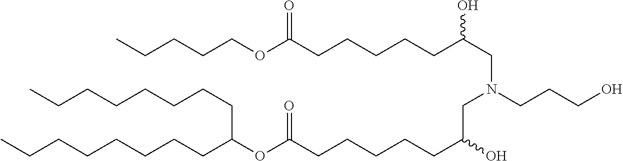 [SMILES: CCCCCCCCC(CCCCCCCC)OC(=O)CCCCCC(O)CN(CCCO)CC(O)CCCCCC(=O)OCCCCC] |
| 164 | 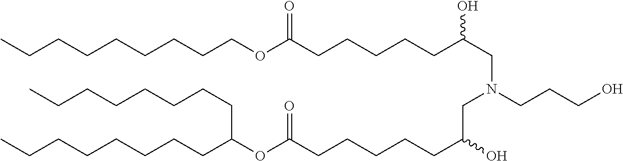 [SMILES: CCCCCCCCCOC(=O)CCCCCC(O)CN(CCCO)CC(O)CCCCCC(=O)OC(CCCCCCCC)CCCCCCCC] |
| 165 | 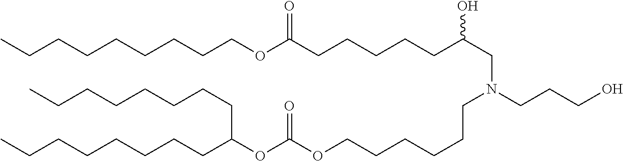 [SMILES: CCCCCCCCCOC(=O)CCCCCC(O)CN(CCCO)CCCCCCOC(=O)OC(CCCCCCCC)CCCCCCCC] |
| 166 | 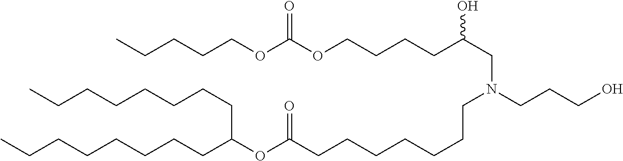 [SMILES: CCCCCCCCC(CCCCCCCC)OC(=O)CCCCCCCN(CCCO)CC(O)CCCCOC(=O)OCCCCC] |
| 167 | 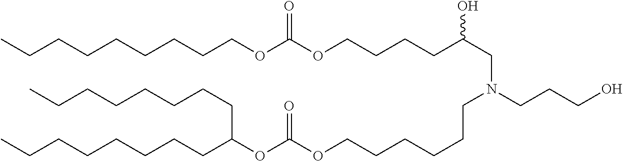 [SMILES: CCCCCCCCCOC(=O)OCCCCC(O)CN(CCCO)CCCCCCOC(=O)OC(CCCCCCCC)CCCCCCCC] |
| 168 | 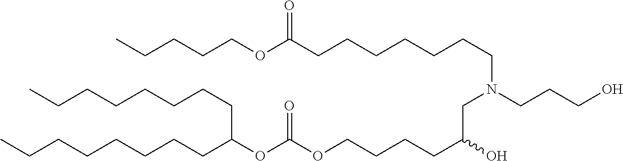 [SMILES: CCCCCCCCC(CCCCCCCC)OC(=O)OCCCCC(O)CN(CCCO)CCCCCCCC(=O)OCCCCC] |
| 169 | 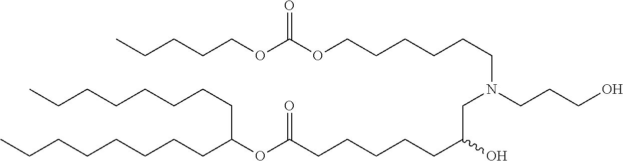 [SMILES: CCCCCCCCC(CCCCCCCC)OC(=O)CCCCCC(O)CN(CCCO)CCCCCCOC(=O)OCCCCC] |
| 170 | 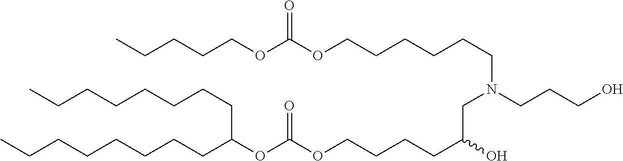 [SMILES: CCCCCCCCC(CCCCCCCC)OC(=O)OCCCCC(O)CN(CCCO)CCCCCCOC(=O)OCCCCC] |
| 171 | 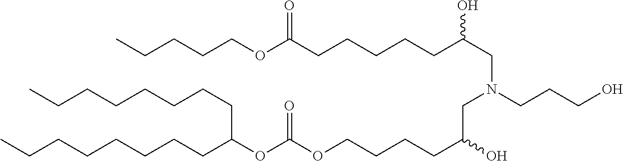 [SMILES: CCCCCCCCC(CCCCCCCC)OC(=O)OCCCCC(O)CN(CCCO)CC(O)CCCCCC(=O)OCCCCC] |
| 172 | 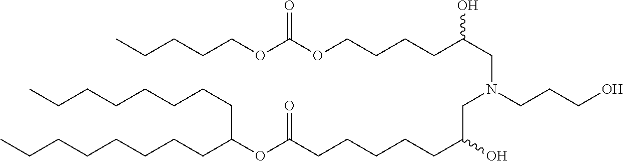 [SMILES: CCCCCCCCC(CCCCCCCC)OC(=O)CCCCCC(O)CN(CCCO)CC(O)CCCCOC(=O)OCCCCC] |
| 173 | 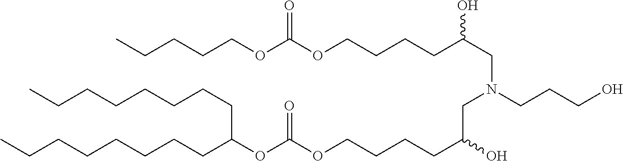 [SMILES: CCCCCCCCC(CCCCCCCC)OC(=O)OCCCCC(O)CN(CCCO)CC(O)CCCCOC(=O)OCCCCC] |
| 174 | 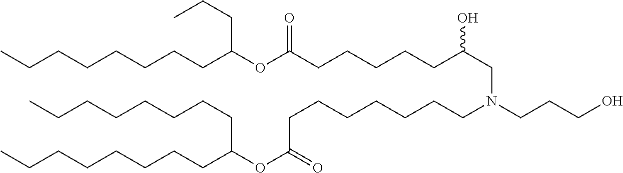 [SMILES: CCCCCCCCC(CCC)OC(=O)CCCCCC(O)CN(CCCO)CCCCCCCC(=O)OC(CCCCCCCC)CCCCCCCC] |
| 175 | 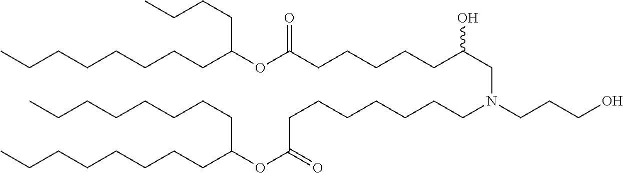 [SMILES: CCCCCCCCC(CCCC)OC(=O)CCCCCC(O)CN(CCCO)CCCCCCCC(=O)OC(CCCCCCCC)CCCCCCCC] |
| 176 | 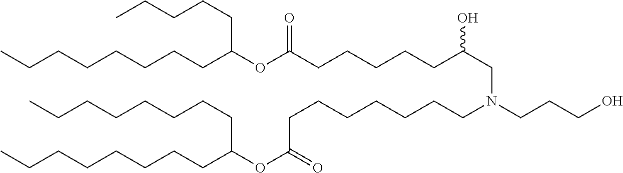 [SMILES: CCCCCCCCC(CCCCC)OC(=O)CCCCCC(O)CN(CCCO)CCCCCCCC(=O)OC(CCCCCCCC)CCCCCCCC] |
| 177 | 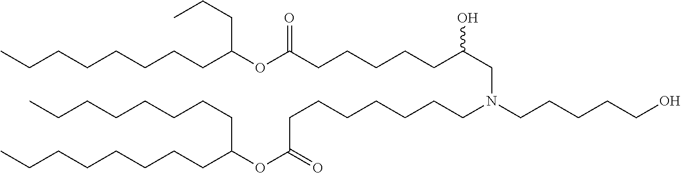 [SMILES: CCCCCCCCC(CCC)OC(=O)CCCCCC(O)CN(CCCCCO)CCCCCCCC(=O)OC(CCCCCCCC)CCCCCCCC] |
| 178 | 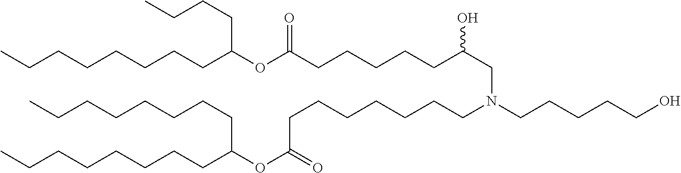 [SMILES: CCCCCCCCC(CCCC)OC(=O)CCCCCC(O)CN(CCCCCO)CCCCCCCC(=O)OC(CCCCCCCC)CCCCCCCC] |
| 179 | 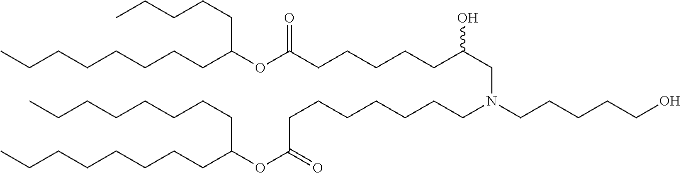 [SMILES: CCCCCCCCC(CCCCC)OC(=O)CCCCCC(O)CN(CCCCCO)CCCCCCCC(=O)OC(CCCCCCCC)CCCCCCCC] |
| 180 | 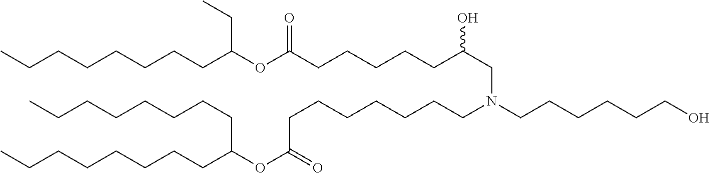 [SMILES: CCCCCCCCC(CC)OC(=O)CCCCCC(O)CN(CCCCCCO)CCCCCCCC(=O)OC(CCCCCCCC)CCCCCCCC] |
| 181 | 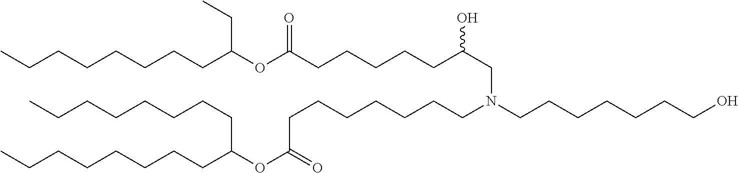 [SMILES: CCCCCCCCC(CC)OC(=O)CCCCCC(O)CN(CCCCCCCO)CCCCCCCC(=O)OC(CCCCCCCC)CCCCCCCC] |
| 182 | 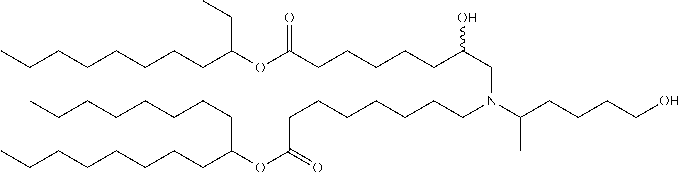 [SMILES: CCCCCCCCC(CC)OC(=O)CCCCCC(O)CN(CCCCCCCC(=O)OC(CCCCCCCC)CCCCCCCC)C(C)CCCCO] |
| 183 | 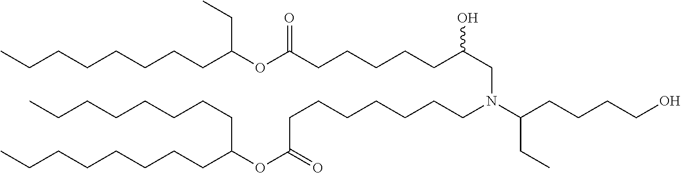 [SMILES: CCCCCCCCC(CC)OC(=O)CCCCCC(O)CN(CCCCCCCC(=O)OC(CCCCCCCC)CCCCCCCC)C(CC)CCCCO] |
| 184 | 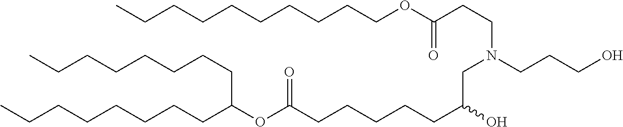 [SMILES: CCCCCCCCCCOC(=O)CCN(CCCO)CC(O)CCCCCC(=O)OC(CCCCCCCC)CCCCCCCC] |
| 185 | 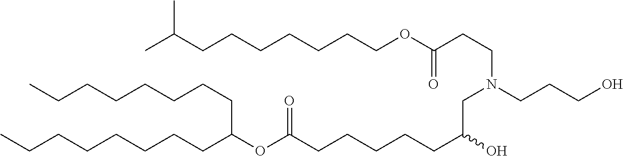 [SMILES: CCCCCCCCC(CCCCCCCC)OC(=O)CCCCCC(O)CN(CCCO)CCC(=O)OCCCCCCCC(C)C] |

In some embodiments, an ionizable lipid is a compound of Formula (15):

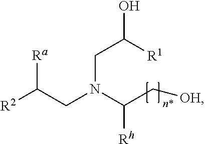

or is a pharmaceutically acceptable salt thereof, wherein:
   n* is an integer from 1 to 7;
   R^{a} is hydrogen or hydroxyl;
   R^{h} is hydrogen or C₁-C₆ alkyl;
   R¹ is C₁-C₃₀ alkyl or R¹*;
   R² is C₁-C₃₀ alkyl or R²*
   R¹* and R²* are independently selected from:
      —(CH₂)_{q}C(O)O(CH₂)ᵣC(R⁸)(R⁹)(R¹⁰),
      —(CH₂)_{q}OC(O)(CH₂)ᵣC(R⁸)(R⁹)(R¹⁰), and
      —(CH₂)_{q}OC(O)O(CH₂)ᵣC(R⁸)(R⁹)(R¹⁰);
   wherein:
      q is an integer from 0 to 12,
      r is an integer from 0 to 6, wherein at least one occurrence of r is not 0;
      R⁸ is H or R¹¹;
      R⁹, R¹⁰, and R¹¹ are each independently C₁-C₂₀ alkyl or C₂-C₂₀-alkenyl; and
   wherein (i) R¹ is R¹*, (ii) R² is R²*, or (iii) R¹ is R¹* and R² is R²*.

In some embodiments of Formula (15), R^{a} is hydrogen and the ionizable lipid is of Formula (16):

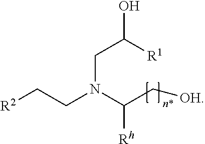

or is a pharmaceutically acceptable salt thereof, wherein:
n* is an integer from 1 to 7.

In some embodiments of Formula (16), the ionizable lipid is of Formula (17):

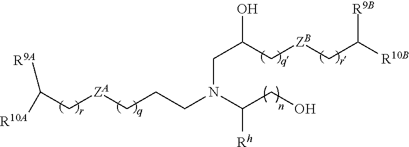

or a pharmaceutically acceptable salt thereof, wherein:
   n is an integer from 1 to 7;
   q and q′ are each independently integers from 0 to 12;
   r and r′ are each independently integers from 0 to 6, wherein at least one of r or r′ is not 0;
   Z^{A} and Z^{B} are each independently selected from \hat{ }—C(O)O—, \hat{ }—OC(O), and —OC(O)O—; where \hat{ }denotes the attachment point to —(CH₂)_{q}— or —(CH₂)_{q′}—; and
   R^{9A}, R^{9B}, R^{10A}, and R^{10B} are each independently C₁-C₂₀ alkyl or C₂-C₂₀ alkenyl.

In some embodiments of Formula (17), Z^{A} and Z^{B} are \hat{ }—C(O)O—, and the ionizable lipid is of Formula (17a-1)

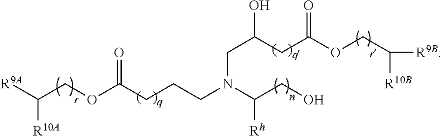

In some embodiments of Formula (17), Z^{A} and Z^{B} are \hat{ }—OC(O)—, and the ionizable lipid is of Formula (17a-2)

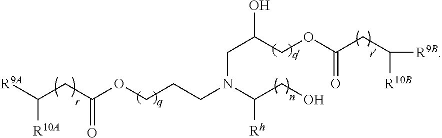

In some embodiments of Formula (17), Z^{A} and Z^{B} are —O(C)(O)O—, and the ionizable lipid is represented by Formula (17a-3):

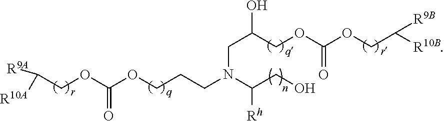

In some embodiments of Formula (15), R^{a} is hydroxyl and the ionizable lipid is of Formula (18):

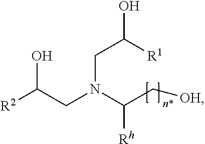

or is a pharmaceutically acceptable salt thereof, wherein:
   n* is an integer from 1 to 7;
   R^{h} is hydrogen or C₁-C₆ alkyl;
   R¹ is C₁-C₃₀ alkyl or R¹*;
   R² is C₁-C₃₀ alkyl or R²*
   R¹* and R²* are independently selected from:
   —(CH₂)_{q}C(O)O(CH₂)ᵣC(R⁸)(R⁹)(R¹⁰),
   —(CH₂)_{q}OC(O)(CH₂)ᵣC(R⁸)(R⁹)(R¹⁰), and
   —(CH₂)_{q}OC(O)O(CH₂)ᵣC(R⁸)(R⁹)(R¹⁰);
wherein:
   q is an integer from 0 to 12,
   r is an integer from 0 to 6, wherein at least one occurrence of r is not 0;
   R⁸ is hydrogen or R¹¹;
   R⁹, R¹⁰, and R¹¹ are each independently C₁-C₂₀ alkyl or C₂-C₂₀-alkenyl;
wherein (i) R¹ is R¹*, (ii) R² is R²*, or (iii) R¹ is R¹* and R² is R²*; and
wherein, for (iii), (a) R¹* and R²* are different or (b) R⁹ and R¹⁰ have different numbers of carbon atoms for at least one of R¹* and R²*

In some embodiments of Formula (18), the ionizable lipid of is of Formula (19):

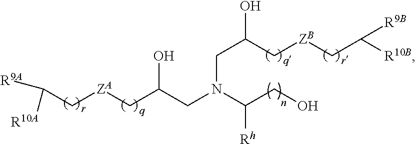

or is a pharmaceutically acceptable salt thereof, wherein:
   n is an integer from 1 to 7;
   q and q′ are each independently integers from 0 to 12;
   r and r′ are each independently integers from 0 to 6, wherein at least one of r or r′ is not 0;
   Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}; and
   R^{9A}, R^{9B}, R^{10A}, and R^{10B} are each independently C₁-C₂₀ alkyl or C₂-C₂₀ alkenyl.

In some embodiments of Formula (19), Z^{A} and Z^{B} are \hat{ }—C(O)O—, and the ionizable lipid is of Formula (19a-1):

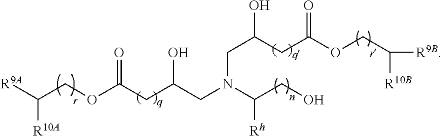

In some embodiments of Formula (19), Z^{A} and Z^{B} are \hat{ }—OC(O)—, and the ionizable lipid is of Formula (19a-2):

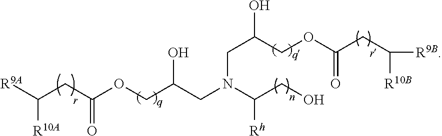

In some embodiments of Formula (19), Z^{A} and Z^{B} are —O(C)(O)O—, and the ionizable lipid is represented by Formula (19a-3):

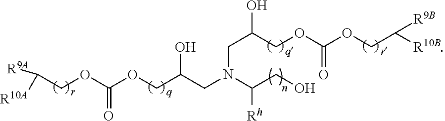

In some embodiments of Formula (15), R¹ is C₁-C₃₀ alkyl, and the ionizable lipid is of Formula (20):

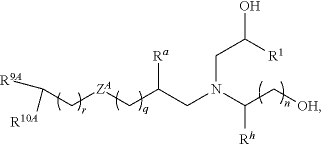

or is a pharmaceutically acceptable salt thereof, wherein:
   Z^{A} is selected from \hat{ }—C(O)O—, \hat{ }—OC(O)—, and —OC(O)O—; where \hat{ } denotes the attachment point to —(CH₂)_{q}—;
   R^{9A} and R^{10A} are each independently C₁-C₂₀ alkyl or C₂-C₂₀ alkenyl;
   n is an integer from 1 to 7;
   q is an integer from 0 to 12; and
   r is an integer from 1 to 6.

In some embodiments of Formula (20), Z^{A} is \hat{ }—C(O)O—, and the ionizable lipid is of Formula (20a-1):

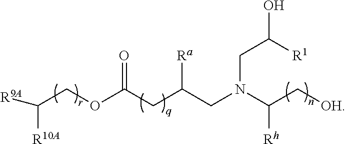

In some embodiments of Formula (20), Z^{A} is \hat{ }—OC(O)—, and the ionizable lipid is of Formula (20a-2):

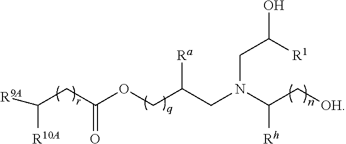

In some embodiments of Formula (20), Z^{A} is —OC(O)O—, and the ionizable lipid is of Formula (20a-3):

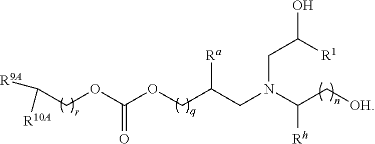

In some embodiments of Formula (15), R² is C₁-C₃₀ alkyl, and the ionizable lipid is of Formula (21):

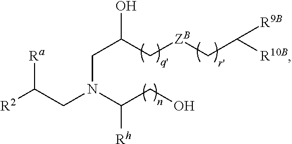

or is a pharmaceutically acceptable salt thereof, wherein:
   Z^{B} is selected from \hat{ }—C(O)O—, \hat{ }—OC(O)—, and —OC(O)O—; where \hat{ }denotes the attachment point to —(CH₂)_{q′}—;
   R^{9B} and R^{10B} are each independently C₁-C₂₀ alkyl or C₂-C₂₀ alkenyl;
   n is an integer from 1 to 7;
   q′ is an integer from 0 to 12; and
   r′ is an integer from 1 to 6.

In some embodiments of Formula (21), Z^{B} is \hat{ }—C(O)O—, and the ionizable lipid is of Formula (21a-1):

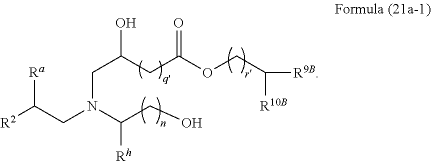

In some embodiments of Formula (21), Z^{B} is \hat{ }—OC(O)—, and the ionizable lipid is of Formula (21a-2):

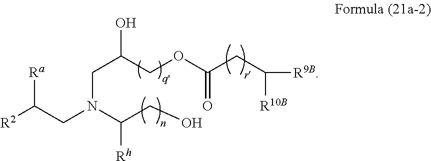

In some embodiments of Formula (21), Z^{B} is —OC(O)O—, and the ionizable lipid is of Formula (21a-3):

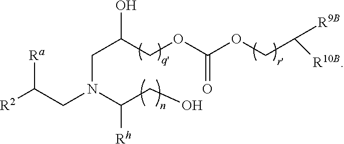

In some embodiments, an ionizable lipid is selected from Table A below:

TABLE A

| Ionizable | |
|---|---|
| lipid | |
| number | Structure |
| 1-a | 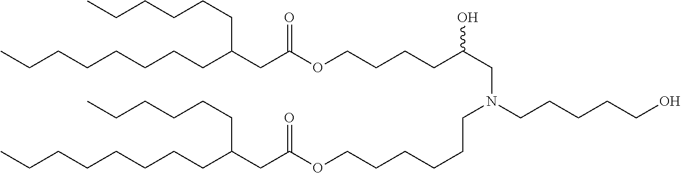 [SMILES: CCCCCCCCC(CCCCCC)CC(=O)OCCCCCCN(CCCCCO)CC(O)CCCCOC(=O)CC(CCCCCC)CCCCCCCC] |
| 1-b | 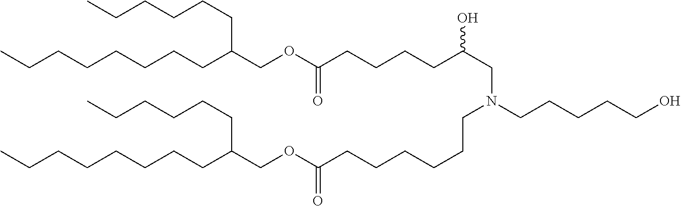 [SMILES: CCCCCCCCC(CCCCCC)COC(=O)CCCCCCN(CCCCCO)CC(O)CCCCC(=O)OCC(CCCCCC)CCCCCCCC] |
| 1-c | 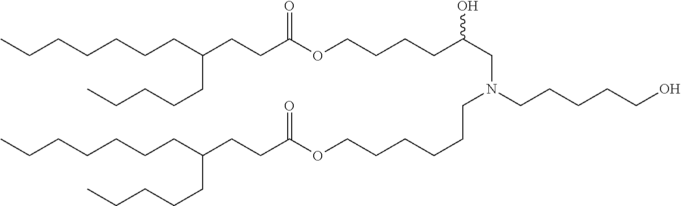 [SMILES: CCCCCCCC(CCCCC)CCC(=O)OCCCCCCN(CCCCCO)CC(O)CCCCOC(=O)CCC(CCCCC)CCCCCCC] |
| 1-d | 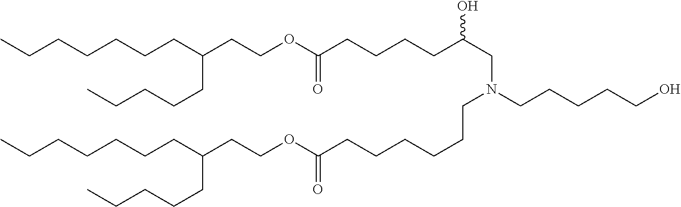 [SMILES: CCCCCCCC(CCCCC)CCOC(=O)CCCCCCN(CCCCCO)CC(O)CCCCC(=O)OCCC(CCCCC)CCCCCCC] |
| 1-e | 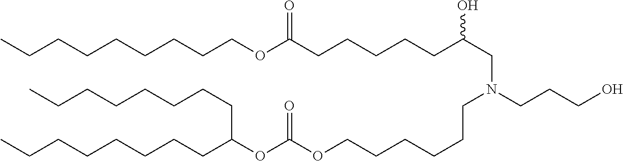 [SMILES: CCCCCCCCCOC(=O)CCCCCC(O)CN(CCCO)CCCCCCOC(=O)OC(CCCCCCCC)CCCCCCCC] |
| 1-f | 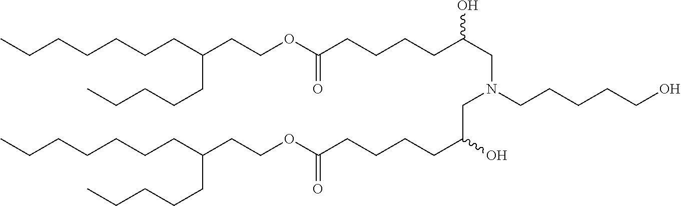 [SMILES: CCCCCCCC(CCCCC)CCOC(=O)CCCCC(O)CN(CCCCCO)CC(O)CCCCC(=O)OCCC(CCCCC)CCCCCCC] |
| 1-g | 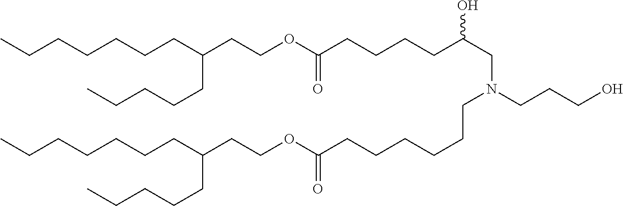 [SMILES: CCCCCCCC(CCCCC)CCOC(=O)CCCCCCN(CCCO)CC(O)CCCCC(=O)OCCC(CCCCC)CCCCCCC] |
| 1-h | 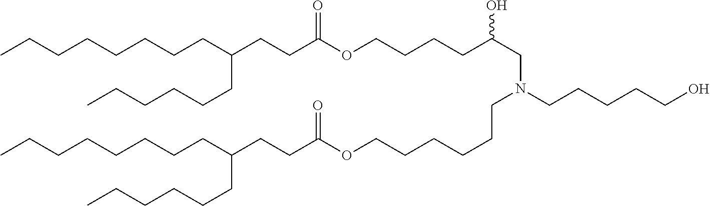 [SMILES: CCCCCCCCC(CCCCCC)CCC(=O)OCCCCCCN(CCCCCO)CC(O)CCCCOC(=O)CCC(CCCCCC)CCCCCCCC] |
| 1-i | 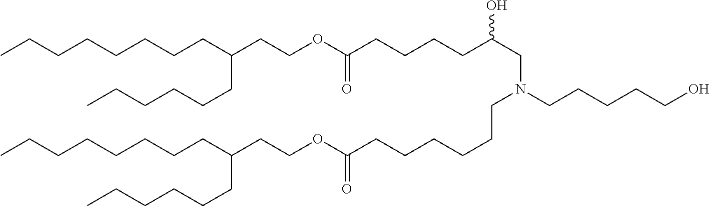 [SMILES: CCCCCCCCC(CCCCCC)CCOC(=O)CCCCCCN(CCCCCO)CC(O)CCCCC(=O)OCCC(CCCCCC)CCCCCCCC] |
| 1-j | 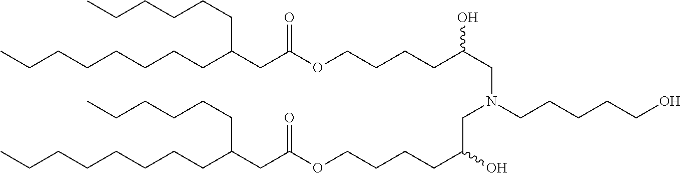 [SMILES: CCCCCCCCC(CCCCCC)CC(=O)OCCCCC(O)CN(CCCCCO)CC(O)CCCCOC(=O)CC(CCCCCC)CCCCCCCC] |
| 1-k | 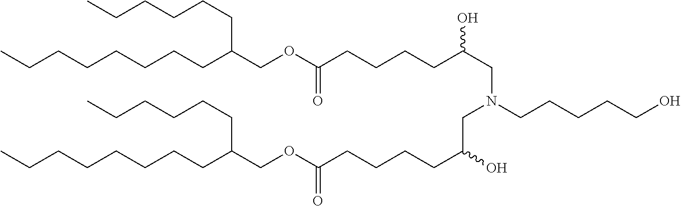 [SMILES: CCCCCCCCC(CCCCCC)COC(=O)CCCCC(O)CN(CCCCCO)CC(O)CCCCC(=O)OCC(CCCCCC)CCCCCCCC] |
| 1-l | 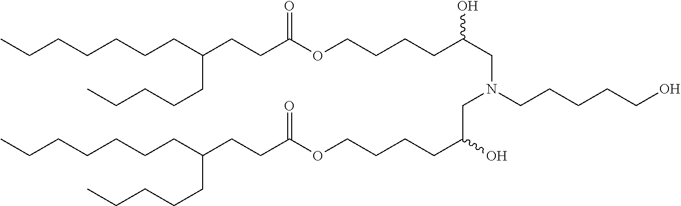 [SMILES: CCCCCCCC(CCCCC)CCC(=O)OCCCCC(O)CN(CCCCCO)CC(O)CCCCOC(=O)CCC(CCCCC)CCCCCCC] |
| 1-m | 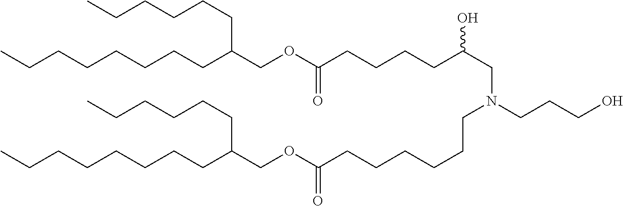 [SMILES: CCCCCCCCC(CCCCCC)COC(=O)CCCCCCN(CCCO)CC(O)CCCCC(=O)OCC(CCCCCC)CCCCCCCC] |
| 1-m | 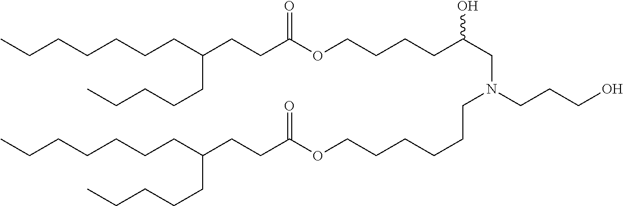 [SMILES: CCCCCCCC(CCCCC)CCC(=O)OCCCCCCN(CCCO)CC(O)CCCCOC(=O)CCC(CCCCC)CCCCCCC] |
| 1-n | 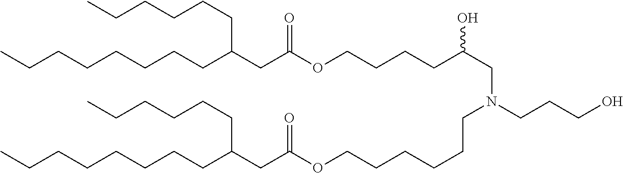 [SMILES: CCCCCCCCC(CCCCCC)CC(=O)OCCCCCCN(CCCO)CC(O)CCCCOC(=O)CC(CCCCCC)CCCCCCCC] |
| 1-o | 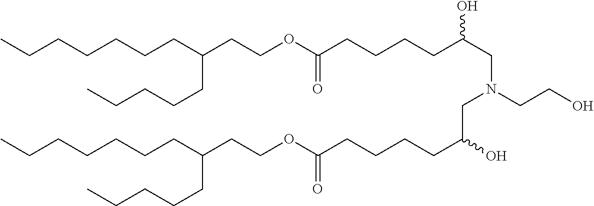 [SMILES: CCCCCCCC(CCCCC)CCOC(=O)CCCCC(O)CN(CCO)CC(O)CCCCC(=O)OCCC(CCCCC)CCCCCCC] |
| 1-p | 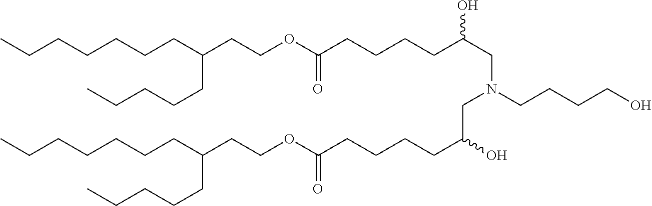 [SMILES: CCCCCCCC(CCCCC)CCOC(=O)CCCCC(O)CN(CCCCO)CC(O)CCCCC(=O)OCCC(CCCCC)CCCCCCC] |
| 1-q | 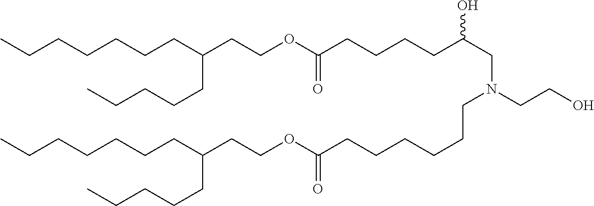 [SMILES: CCCCCCCC(CCCCC)CCOC(=O)CCCCCCN(CCO)CC(O)CCCCC(=O)OCCC(CCCCC)CCCCCCC] |
| 1-r | 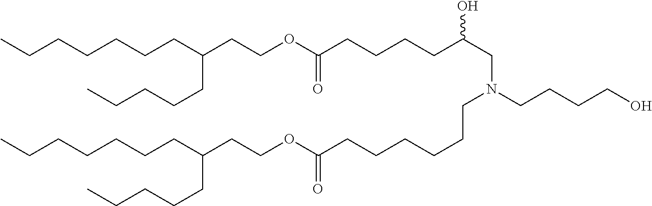 [SMILES: CCCCCCCC(CCCCC)CCOC(=O)CCCCCCN(CCCCO)CC(O)CCCCC(=O)OCCC(CCCCC)CCCCCCC] |
| 1-s | 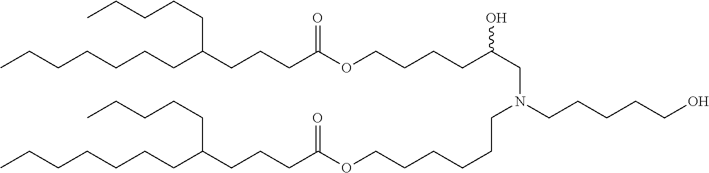 [SMILES: CCCCCCCC(CCCCC)CCCC(=O)OCCCCCCN(CCCCCO)CC(O)CCCCOC(=O)CCCC(CCCCC)CCCCCCC] |
| 1-t | 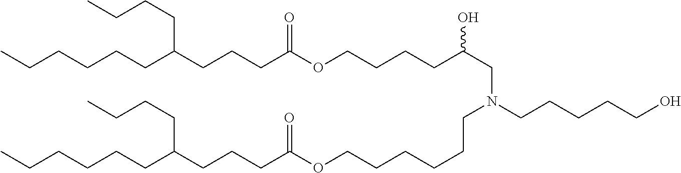 [SMILES: CCCCCCC(CCCC)CCCC(=O)OCCCCCCN(CCCCCO)CC(O)CCCCOC(=O)CCCC(CCCC)CCCCCC] |
| 1-u | 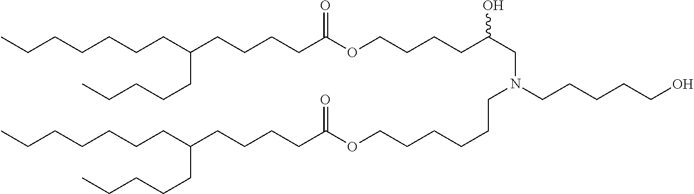 [SMILES: CCCCCCCC(CCCCC)CCCCC(=O)OCCCCCCN(CCCCCO)CC(O)CCCCOC(=O)CCCCC(CCCCC)CCCCCCC] |
| 1-v | 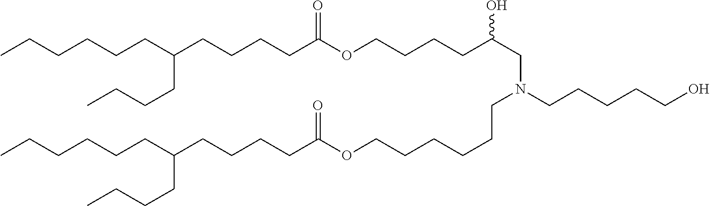 [SMILES: CCCCCCC(CCCC)CCCCC(=O)OCCCCCCN(CCCCCO)CC(O)CCCCOC(=O)CCCCC(CCCC)CCCCCC] |
| 1-w | 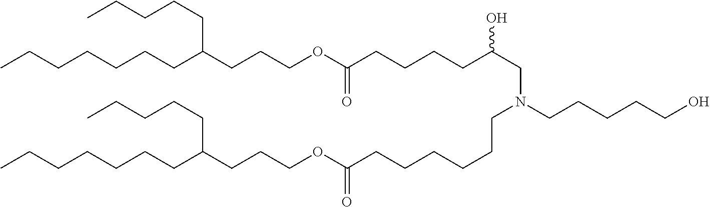 [SMILES: CCCCCCCC(CCCCC)CCCOC(=O)CCCCCCN(CCCCCO)CC(O)CCCCC(=O)OCCCC(CCCCC)CCCCCCC] |
| 1-x | 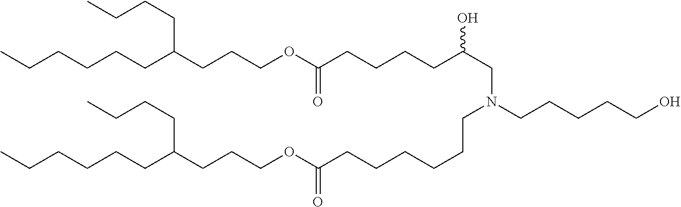 [SMILES: CCCCCCC(CCCC)CCCOC(=O)CCCCCCN(CCCCCO)CC(O)CCCCC(=O)OCCCC(CCCC)CCCCCC] |
| 1-y | 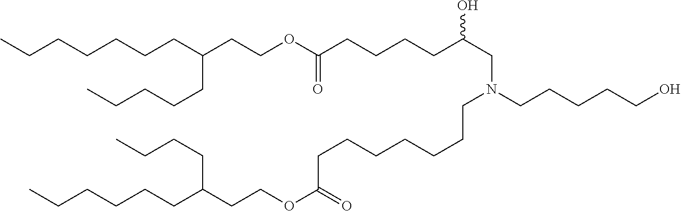 [SMILES: CCCCCCCC(CCCCC)CCOC(=O)CCCCC(O)CN(CCCCCO)CCCCCCCC(=O)OCCC(CCCC)CCCCCC] |
| 1-z | 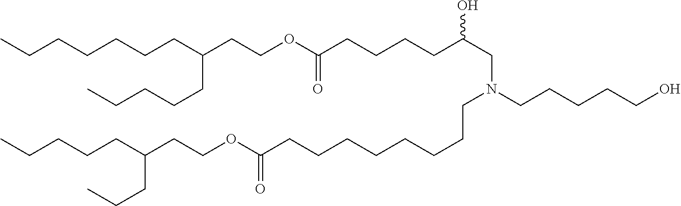 [SMILES: CCCCCCCC(CCCCC)CCOC(=O)CCCCC(O)CN(CCCCCO)CCCCCCCCC(=O)OCCC(CCC)CCCCC] |
| 1-aa | 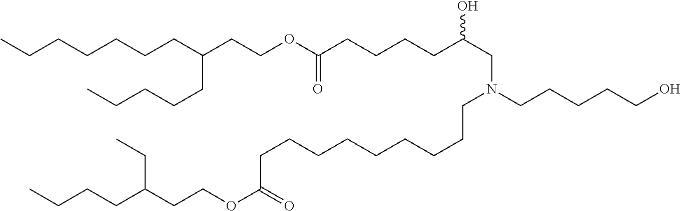 [SMILES: CCCCCCCC(CCCCC)CCOC(=O)CCCCC(O)CN(CCCCCO)CCCCCCCCCC(=O)OCCC(CC)CCCC] |
| 1-ab | 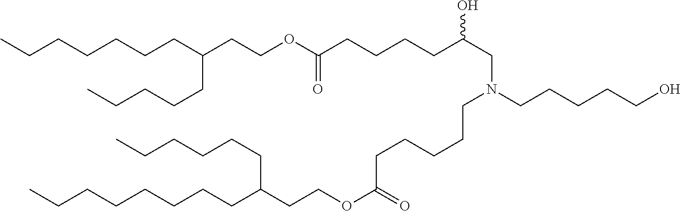 [SMILES: CCCCCCCCC(CCCCCC)CCOC(=O)CCCCCN(CCCCCO)CC(O)CCCCC(=O)OCCC(CCCCC)CCCCCCC] |
| 1-ac | 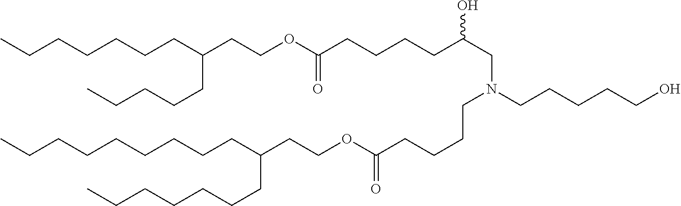 [SMILES: CCCCCCCCCC(CCCCCCC)CCOC(=O)CCCCN(CCCCCO)CC(O)CCCCC(=O)OCCC(CCCCC)CCCCCCC] |
| 1-ad | 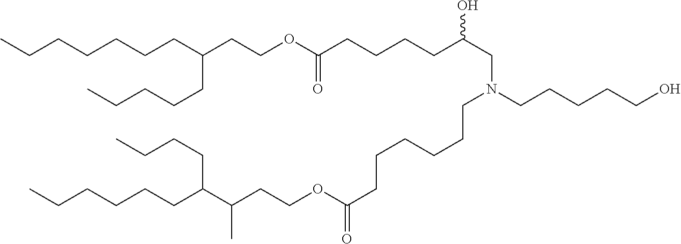 [SMILES: CCCCCCCC(CCCCC)CCOC(=O)CCCCC(O)CN(CCCCCO)CCCCCCC(=O)OCCC(C)C(CCCC)CCCCCC] |
| 1-ae | 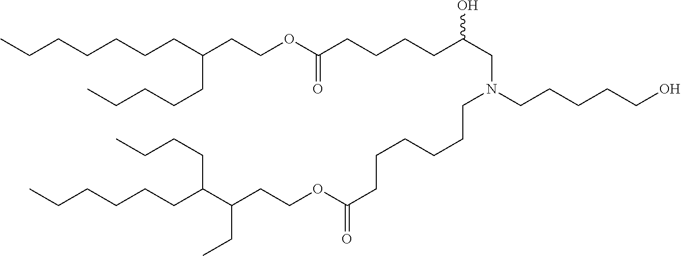 [SMILES: CCCCCCCC(CCCCC)CCOC(=O)CCCCC(O)CN(CCCCCO)CCCCCCC(=O)OCCC(CC)C(CCCC)CCCCCC] |
| 1-af | 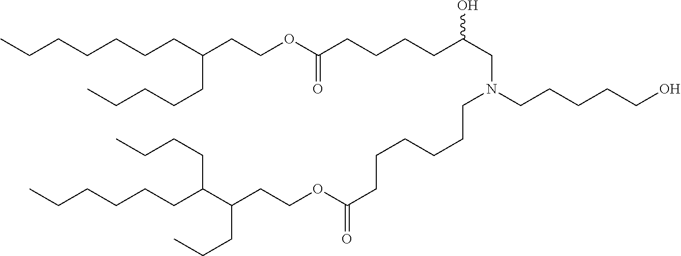 [SMILES: CCCCCCCC(CCCCC)CCOC(=O)CCCCC(O)CN(CCCCCO)CCCCCCC(=O)OCCC(CCC)C(CCCC)CCCCCC] |
| 1-ag | 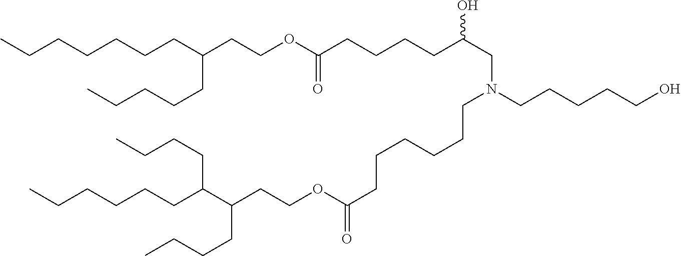 [SMILES: CCCCCCCC(CCCCC)CCOC(=O)CCCCC(O)CN(CCCCCO)CCCCCCC(=O)OCCC(CCCC)C(CCCC)CCCCCC] |
| 1-ah | 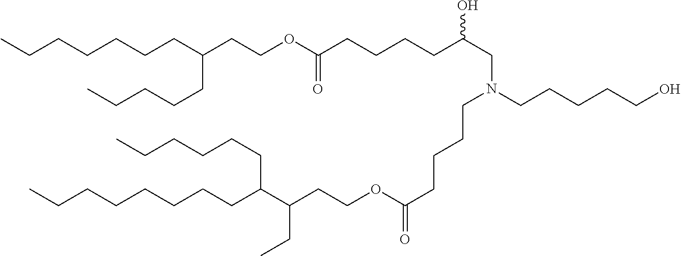 [SMILES: CCCCCCCCC(CCCCCC)C(CC)CCOC(=O)CCCCN(CCCCCO)CC(O)CCCCC(=O)OCCC(CCCCC)CCCCCCC] |
| 1-ai | 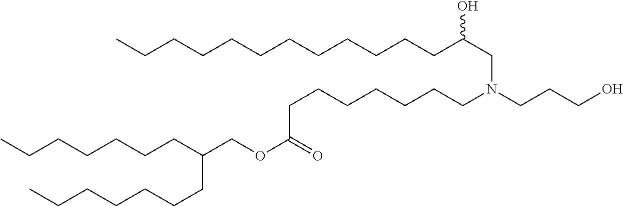 [SMILES: CCCCCCCCCCCCC(O)CN(CCCO)CCCCCCCC(=O)OCC(CCCCCCC)CCCCCCC] |
| 1-aj | 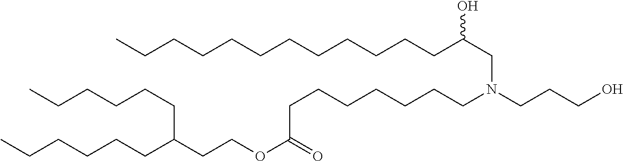 [SMILES: CCCCCCCCCCCCC(O)CN(CCCO)CCCCCCCC(=O)OCCC(CCCCCC)CCCCCC] |
| 1-ak | 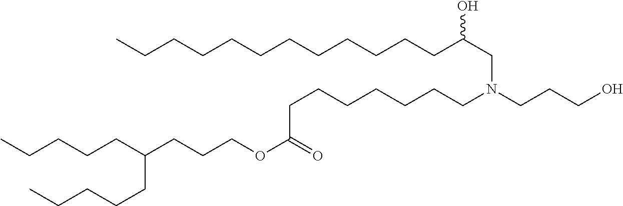 [SMILES: CCCCCCCCCCCCC(O)CN(CCCO)CCCCCCCC(=O)OCCCC(CCCCC)CCCCC] |

In some embodiments, an ionizable lipid of the present disclosure is represented by Formula (22):

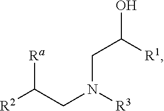

or is a pharmaceutically acceptable salt thereof, wherein:
   R^{a} is hydrogen or hydroxyl;
   R¹ is C₁-C₃₀ alkyl or R¹*;
   R² is C₁-C₃₀ alkyl or R²*
   R¹* and R²* are independently selected from:
      —(CH₂)_{q}C(O)O(CH₂)ᵣC(R⁴)(R⁵)(R⁶),
      —(CH₂)_{q}OC(O)(CH₂)ᵣC(R⁴)(R⁵)(R⁶), and
      —(CH₂)_{q}OC(O)O(CH₂)ᵣC(R⁴)(R⁵)(R⁶);
   wherein:
      q is an integer from 0 to 12,
      r is an integer from 0 to 6, wherein at least one occurrence of r is not 0;
      R⁴ is hydrogen or R⁷;
      R⁵, R⁶, and R⁷ are each independently C₁-C₂₀ alkyl or C₂-C₂₀-alkenyl;
   wherein (i) R¹ is R¹*, (ii) R² is R²*, or (iii) R¹ is R¹* and R² is R²*; and
   R³ is L-R′, wherein L is linear or branched C₁-C₁₀ alkylene, and R′ is (i) mono- or bicyclic heterocyclyl or heteroaryl, such as imidazolyl, pyrazolyl, 1,2,4-triazolyl, or benzimidazolyl, each optionally substituted at one or more available carbon and nitrogen by C₁-C₆ alkyl, or (ii) R^{A}, R^{B}, or R^{C}, wherein
   R^{A} is selected from:

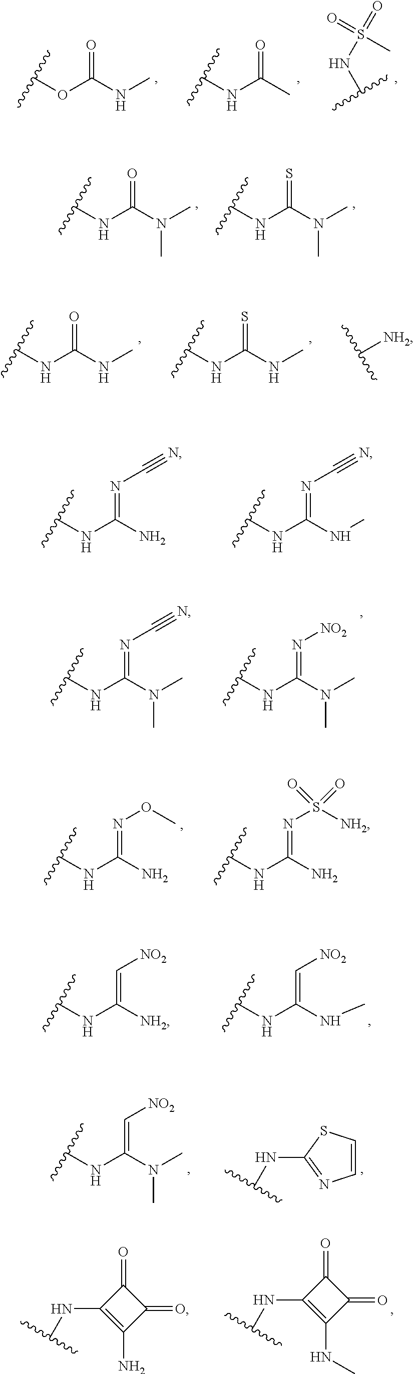

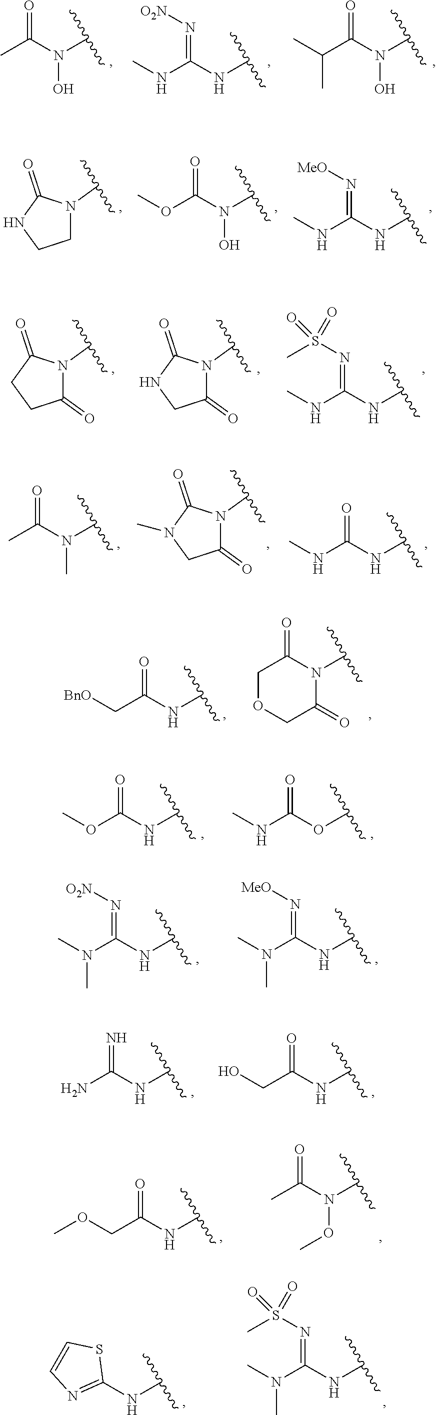

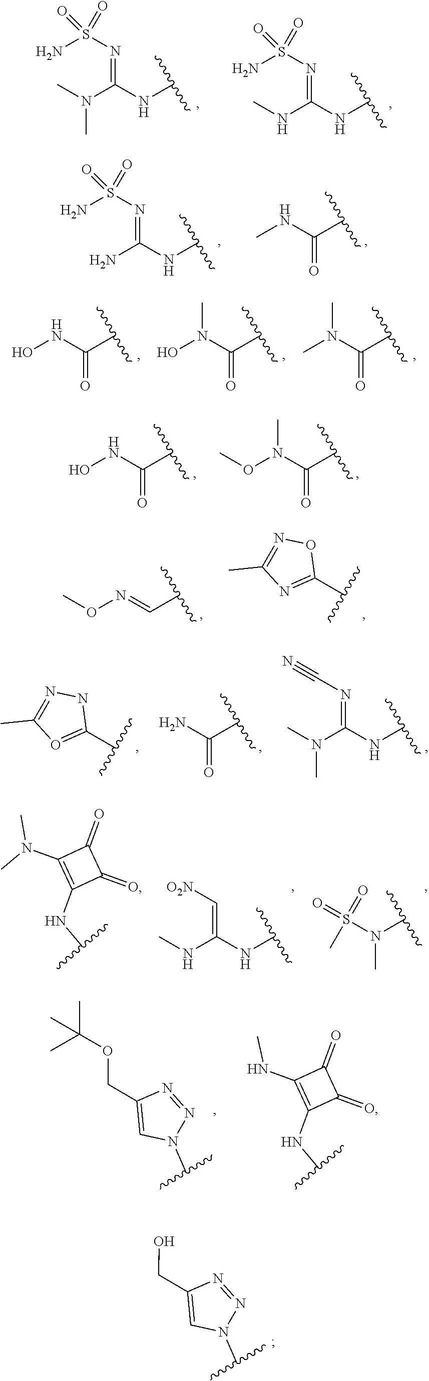

R^{B} is selected from:

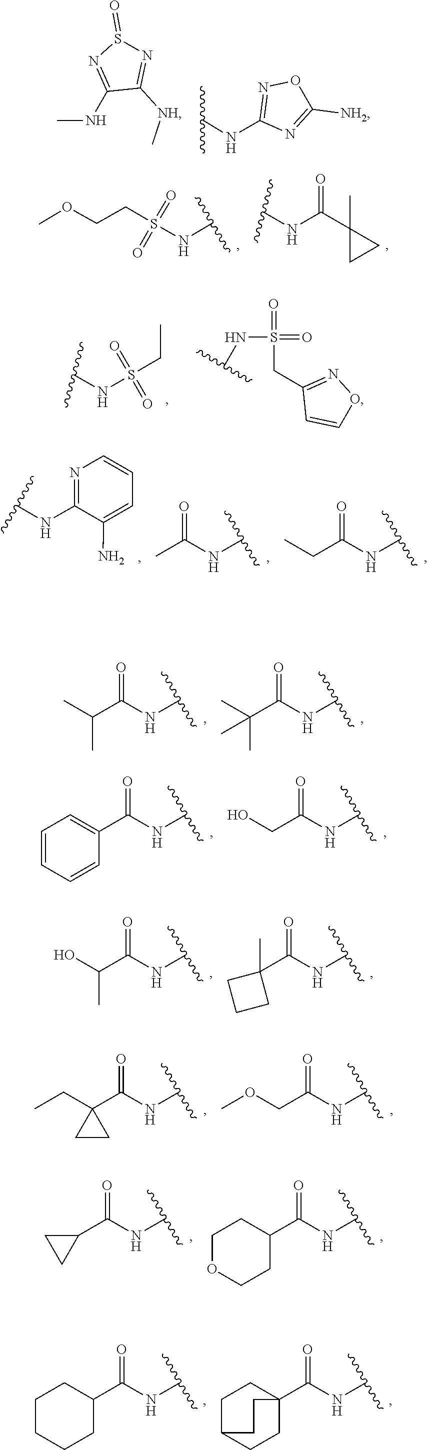

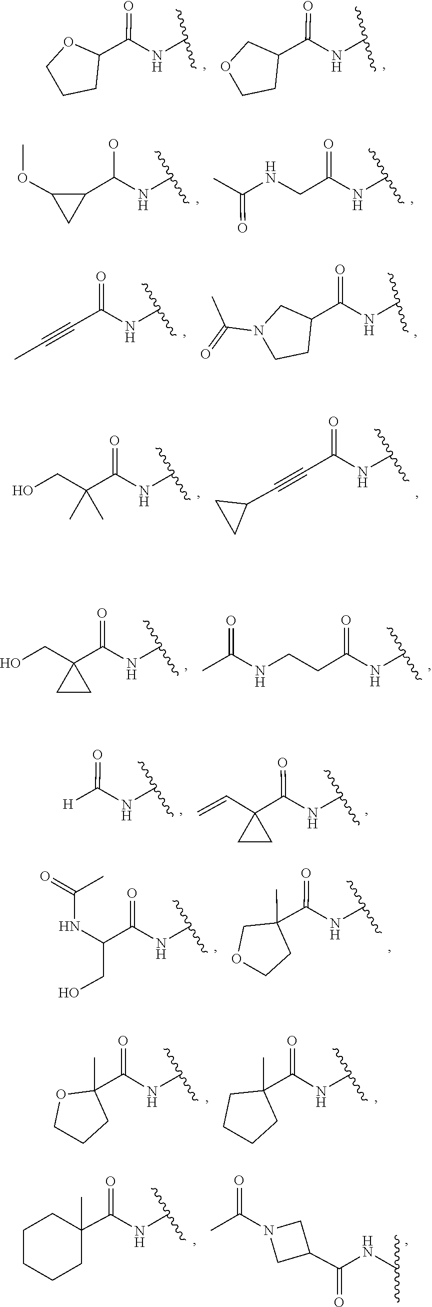

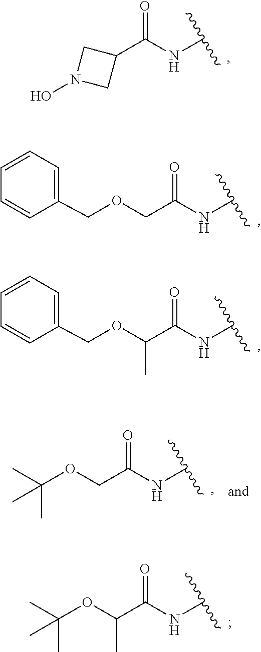

and
R^{C} is selected from:

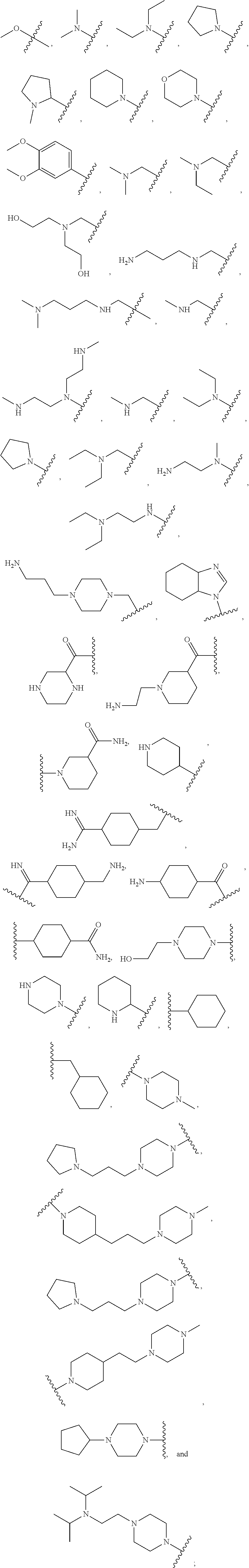

with the proviso that the ionizable lipid is not:

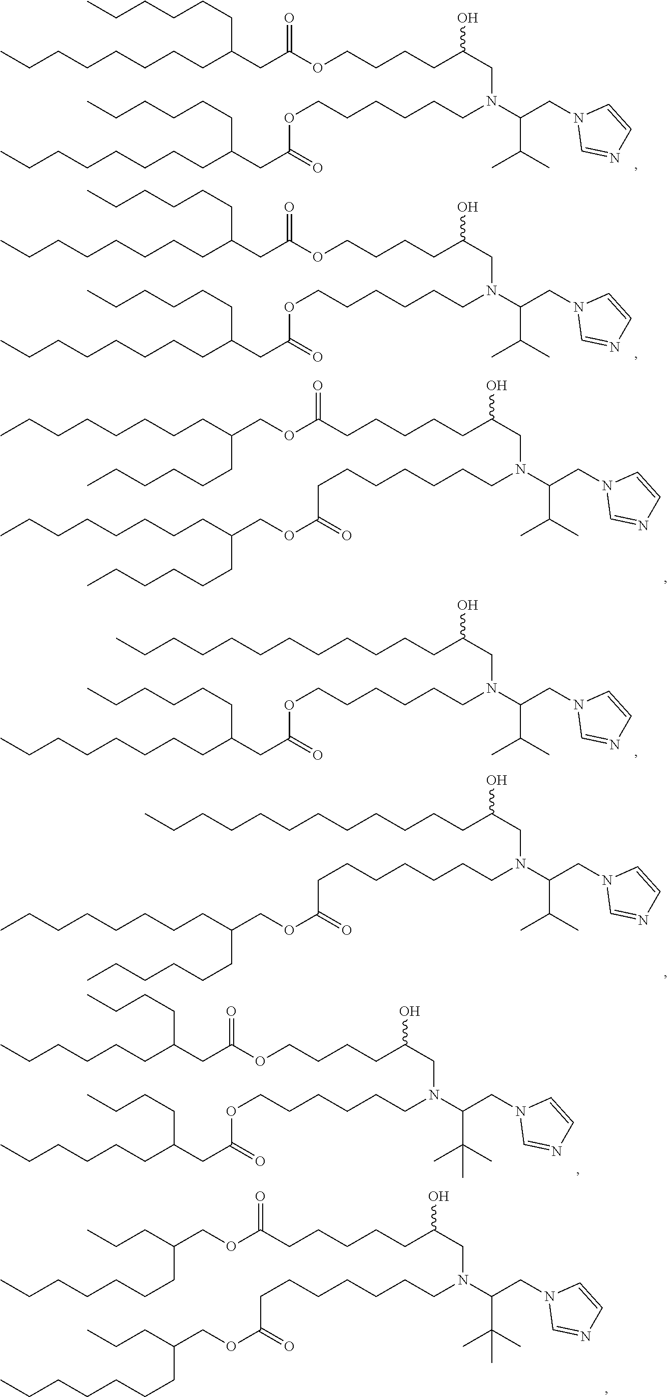

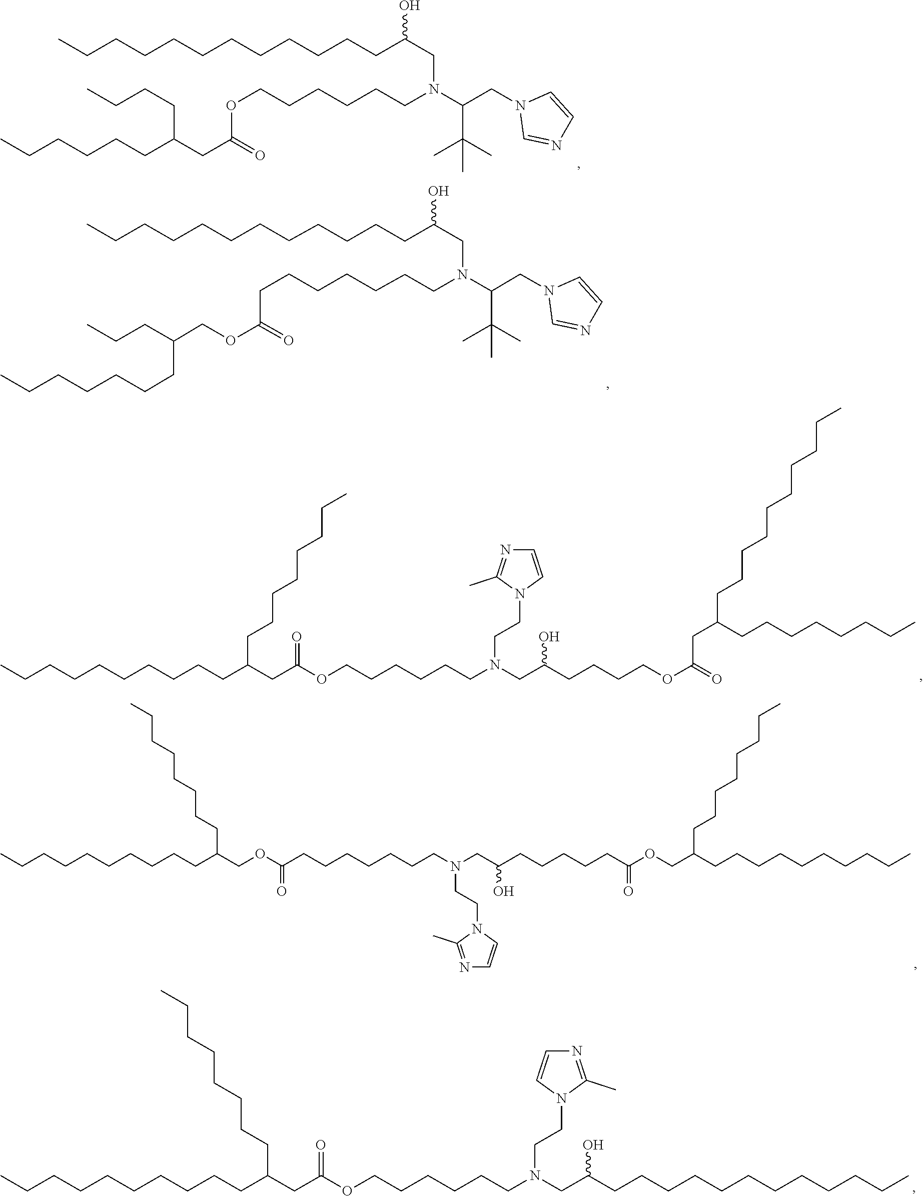

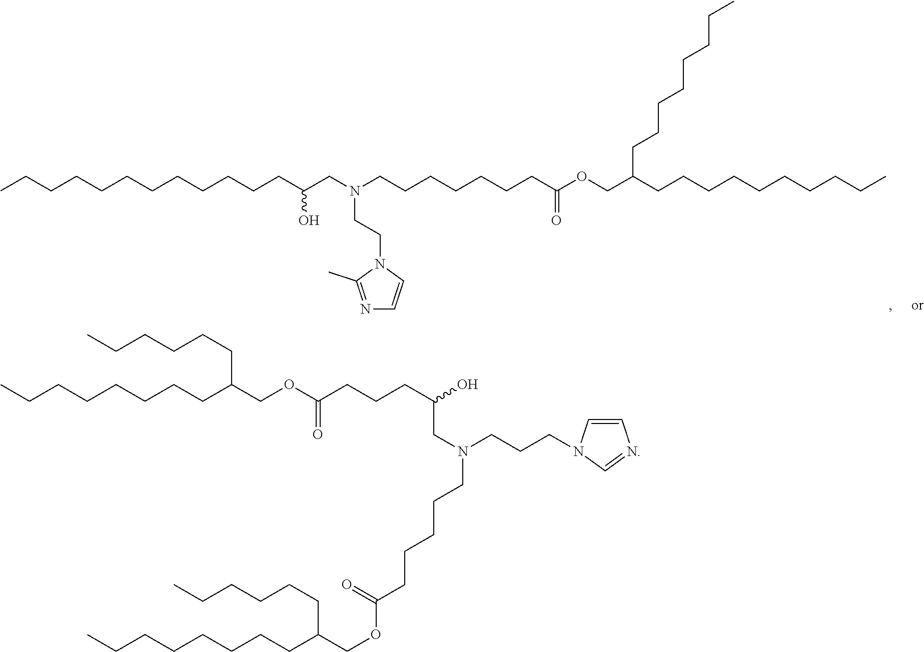

In some embodiments of Formula (22), R³ is selected from:

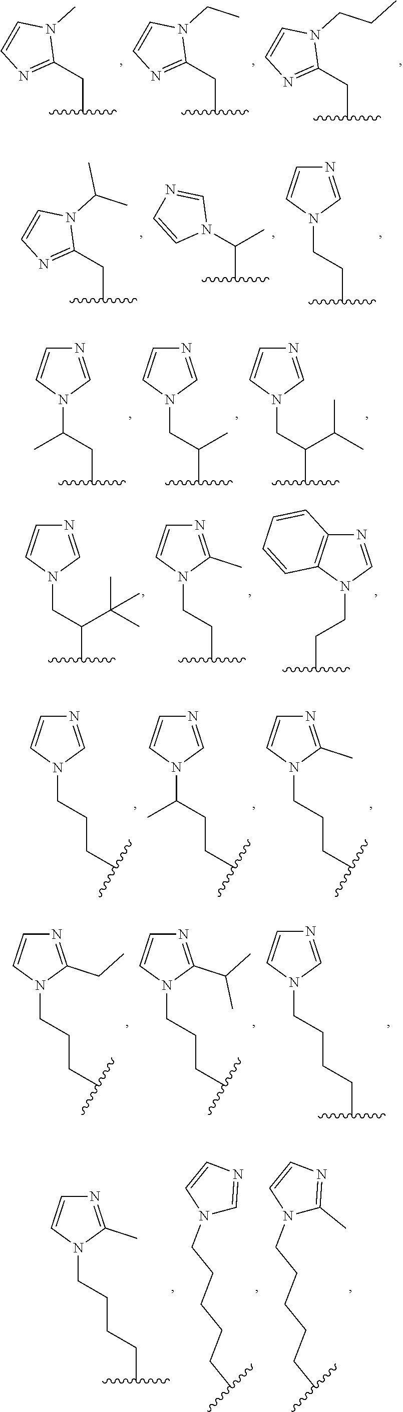

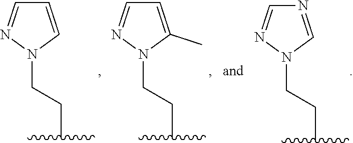

In some embodiments of Formula (22), R¹ is R¹*, R² is R²*, and the ionizable lipid is of Formula (23):

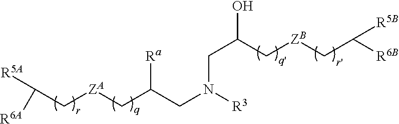

wherein:
q and q′ are each independently integers from 0 to 12;
r and r′ are each independently integers from 0 to 6, wherein at least one of r or r′ is not 0;
Z^{A} and Z^{B} are each independently selected from \hat{ }—(O)O—, \hat{ }—OC(O), and —OC(O)O—; where \hat{ } denotes the attachment point to —(CH₂)_{q}— or —(CH₂)_{q′}—; and
R^{5A}, R^{5B}, R^{6A}, and R^{6B} are each independently C₁-C₂₀ alkyl or C₂-C₂₀ alkenyl.

In some embodiments of Formula (23), Z^{A} and Z^{B} are \hat{ }—(O)O—, and the ionizable lipid is of Formula (23a-1):

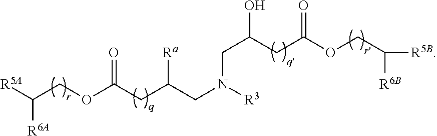

In some embodiments of Formula (23), Z^{A} and Z^{B} are \hat{ }—OC(O)—, and the ionizable lipid is of Formula (23a-2)

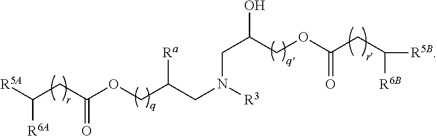

In some embodiments of Formula (23), Z^{A} and Z^{B} are \hat{ }—O(C)(O)O—, and the ionizable lipid is represented by Formula (23a-3):

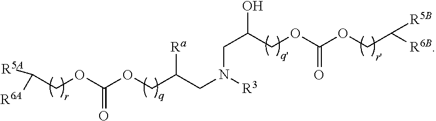

In some embodiments of Formula (22), R² is C₁-C₃₀ alkyl, and the ionizable lipid is of Formula (25):

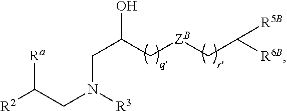

or is a pharmaceutically acceptable salt thereof, wherein:
   Z^{B} is selected from \hat{ }—C(O)O—, \hat{ }—OC(O)—, and —OC(O)O—; where \hat{ } denotes the attachment point to —(CH₂)_{q′}—;
   R^{5B} and R^{6B} are each independently C₁-C₂₀ alkyl or C₂-C₂₀ alkenyl;
   q′ is an integer from 0 to 12; and
   r′ is an integer from 1 to 6.
   In some embodiments of Formula (25), Z^{B} is \hat{ }—C(O)O—, and the ionizable lipid is of Formula (25a-1):

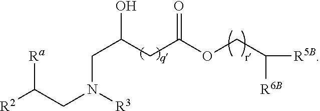

In some embodiments of Formula (25), Z^{B} is \hat{ }—OC(O)—, and the ionizable lipid is of Formula (25a-2):

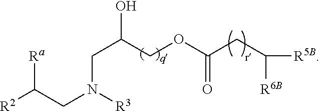

In some embodiments of Formula (25), Z^{B} is —OC(O)O—, and the ionizable lipid is of Formula (25a-3):

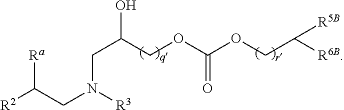

In some embodiments, an ionizable lipid is selected from Table B. below:

TABLE B

| Lipid | Structure |
|---|---|
| 100-a | 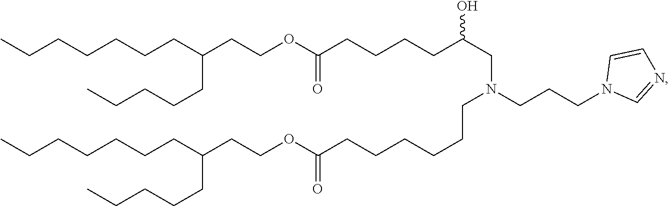 [SMILES: CCCCCCCC(CCCCC)CCOC(=O)CCCCCCN(CCCn1ccnc1)CC(O)CCCCC(=O)OCCC(CCCCC)CCCCCCC] |
| 100-b | 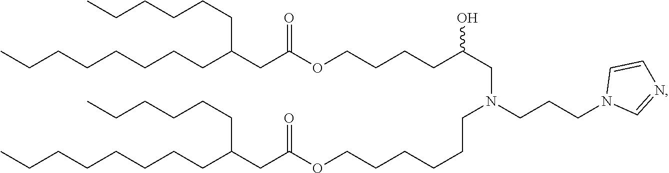 [SMILES: CCCCCCCCC(CCCCCC)CC(=O)OCCCCCCN(CCCn1ccnc1)CC(O)CCCCOC(=O)CC(CCCCCC)CCCCCCCC] |
| 100-c | 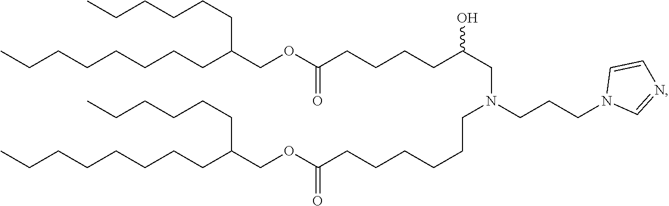 [SMILES: CCCCCCCCC(CCCCCC)COC(=O)CCCCCCN(CCCn1ccnc1)CC(O)CCCCC(=O)OCC(CCCCCC)CCCCCCCC] |
| 100-d | 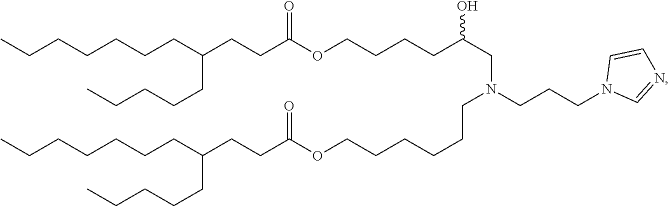 [SMILES: CCCCCCCC(CCCCC)CCC(=O)OCCCCCCN(CCCn1ccnc1)CC(O)CCCCOC(=O)CCC(CCCCC)CCCCCCC] |
| 100-e | 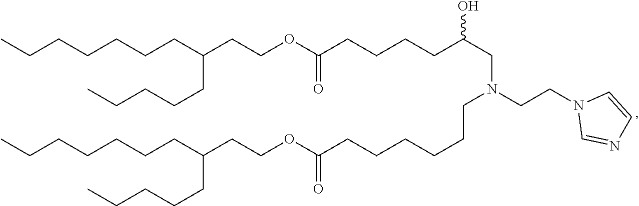 [SMILES: CCCCCCCC(CCCCC)CCOC(=O)CCCCCCN(CCn1ccnc1)CC(O)CCCCC(=O)OCCC(CCCCC)CCCCCCC] |
| | and |
| 100-f | 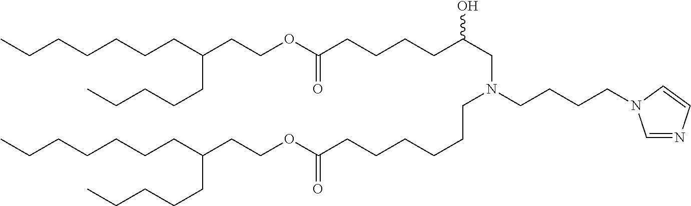 [SMILES: CCCCCCCC(CCCCC)CCOC(=O)CCCCCCN(CCCCn1ccnc1)CC(O)CCCCC(=O)OCCC(CCCCC)CCCCCCC] |

In some embodiments, an ionizable lipid is selected from Table C. below:

TABLE C

| Lipid | Structure |
|---|---|
| 1-al | 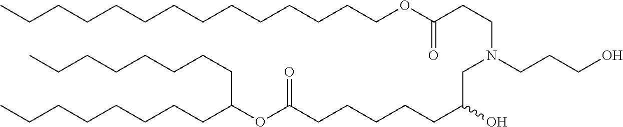 [SMILES: CCCCCCCCCCCCCCOC(=O)CCN(CCCO)CC(O)CCCCCC(=O)OC(CCCCCCCC)CCCCCCCC] |
| 1-am | 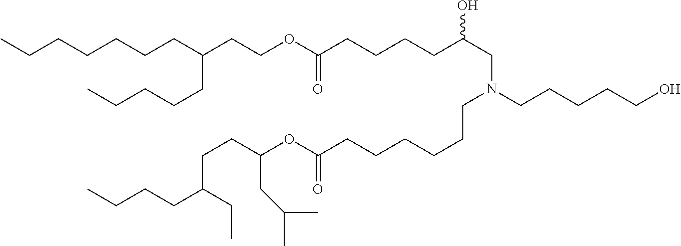 [SMILES: CCCCCCCC(CCCCC)CCOC(=O)CCCCC(O)CN(CCCCCO)CCCCCCC(=O)OC(CCC(CC)CCCC)CC(C)C] |
| 1-an | 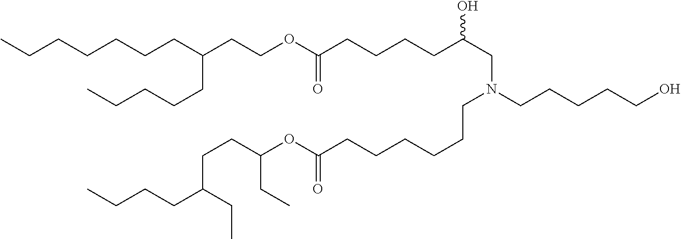 [SMILES: CCCCCCCC(CCCCC)CCOC(=O)CCCCC(O)CN(CCCCCO)CCCCCCC(=O)OC(CC)CCC(CC)CCCC] |
| 1-ao | 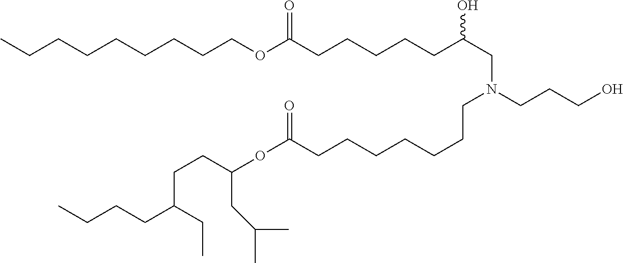 [SMILES: CCCCCCCCCOC(=O)CCCCCC(O)CN(CCCO)CCCCCCCC(=O)OC(CCC(CC)CCCC)CC(C)C] |
| 1-ap | 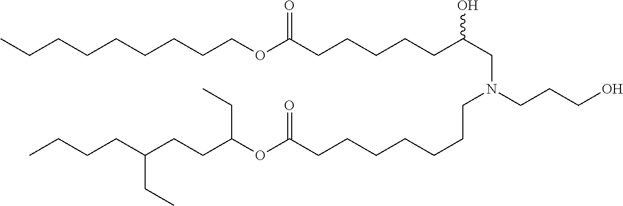 [SMILES: CCCCCCCCCOC(=O)CCCCCC(O)CN(CCCO)CCCCCCCC(=O)OC(CC)CCC(CC)CCCC] |
| 1-aq | 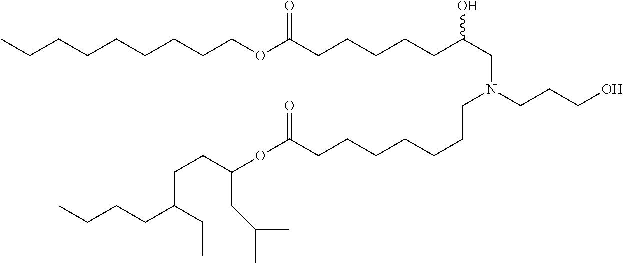 [SMILES: CCCCCCCCCOC(=O)CCCCCC(O)CN(CCCO)CCCCCCCC(=O)OC(CCC(CC)CCCC)CC(C)C] |
| 1-ar | 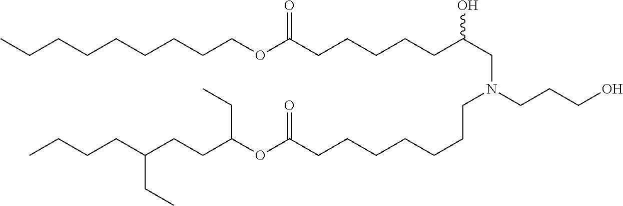 [SMILES: CCCCCCCCCOC(=O)CCCCCC(O)CN(CCCO)CCCCCCCC(=O)OC(CC)CCC(CC)CCCC] |
| 1-as | 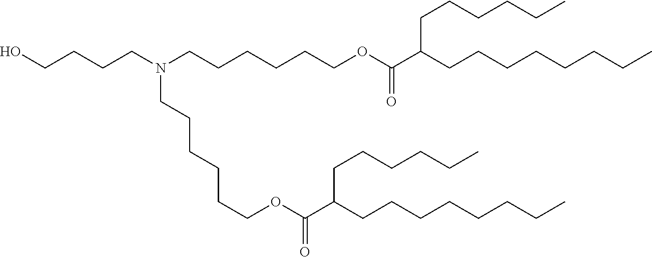 [SMILES: CCCCCCCCC(CCCCCC)C(=O)OCCCCCCN(CCCCO)CCCCCCOC(=O)C(CCCCCC)CCCCCCCC] |
| 1-at | 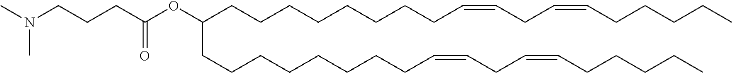 [SMILES: CCCCC/C=C\C/C=C\CCCCCCCCC(CCCCCCCC/C=C\C/C=C\CCCCC)OC(=O)CCCN(C)C] |
| 1-au | 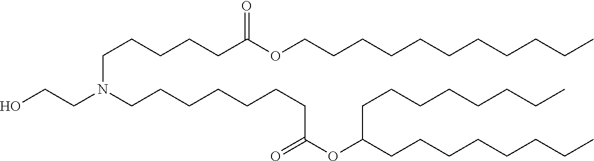 [SMILES: CCCCCCCCCCCOC(=O)CCCCCN(CCO)CCCCCCCC(=O)OC(CCCCCCCC)CCCCCCCC] |

In some embodiments, an LNP of the present disclosure comprises an ionizable lipid disclosed in one of US 2023/0053437; US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, an LNP described herein comprises a lipid, e.g., an ionizable lipid, disclosed in US Application publication US2017/0119904, which is incorporated by reference herein, in its entirety.

In some embodiments, an LNP described herein comprises a lipid, e.g., an ionizable lipid, disclosed in PCT Application publication WO2021/204179, which is incorporated by reference herein, in its entirety.

In some embodiments, an LNP described herein comprises a lipid, e.g., an ionizable lipid, disclosed in PCT Application WO2022/251665A1, which is incorporated by reference herein, in its entirety.

In some embodiments, an LNP described herein comprises an ionizable lipid of Table Z:

TABLE Z
Exemplary Ionizable Lipids

| Compound # | Structure |
|---|---|
| L-1 | 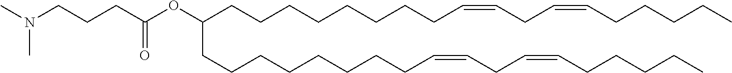 [SMILES: CCCCC/C=C\C/C=C\CCCCCCCCC(CCCCCCCC/C=C\C/C=C\CCCCC)OC(=O)CCCN(C)C] |
| L-2 | 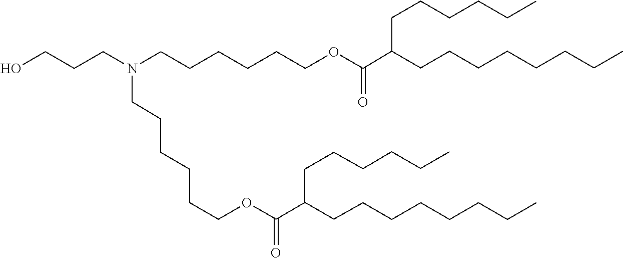 [SMILES: CCCCCCCCC(CCCCCC)C(=O)OCCCCCCN(CCCO)CCCCCCOC(=O)C(CCCCCC)CCCCCCCC] |
| L-3 | 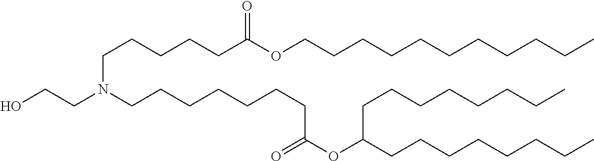 [SMILES: CCCCCCCCCCCOC(=O)CCCCCN(CCO)CCCCCCCC(=O)OC(CCCCCCCC)CCCCCCCC] |
| L-4 | 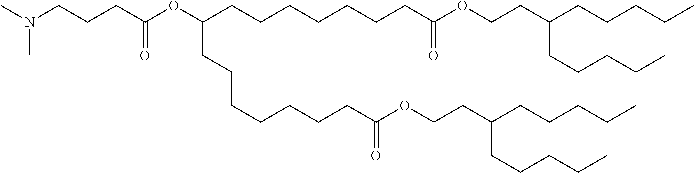 [SMILES: CCCCCC(CCCCC)CCOC(=O)CCCCCCCC(CCCCCCCC(=O)OCCC(CCCCC)CCCCC)OC(=O)CCCN(C)C] |
| L-5 | 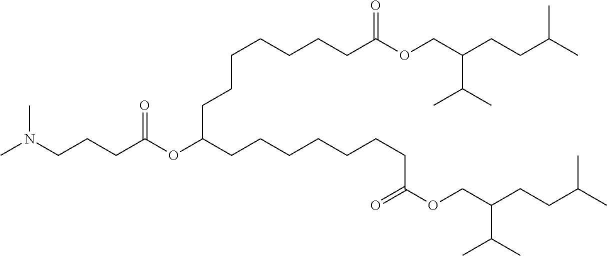 [SMILES: CC(C)CCC(COC(=O)CCCCCCCC(CCCCCCCC(=O)OCC(CCC(C)C)C(C)C)OC(=O)CCCN(C)C)C(C)C] |
| L-6 | 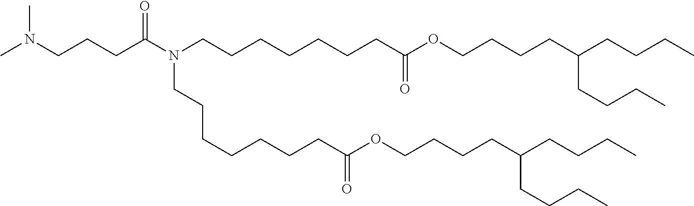 [SMILES: CCCCC(CCCC)CCCCOC(=O)CCCCCCCN(CCCCCCCC(=O)OCCCCC(CCCC)CCCC)C(=O)CCCN(C)C] |
| L-7 | 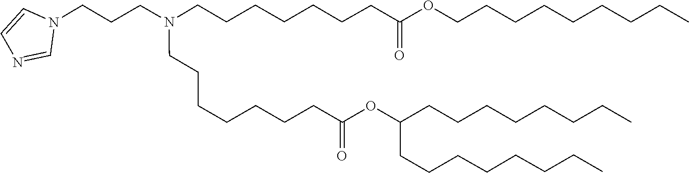 [SMILES: CCCCCCCCCOC(=O)CCCCCCCN(CCCCCCCC(=O)OC(CCCCCCCC)CCCCCCCC)CCCn1ccnc1] |
| L-8 | 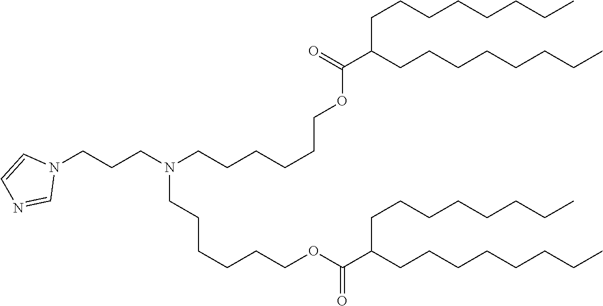 [SMILES: CCCCCCCCC(CCCCCCCC)C(=O)OCCCCCCN(CCCCCCOC(=O)C(CCCCCCCC)CCCCCCCC)CCCn1ccnc1] |
| L-9 | 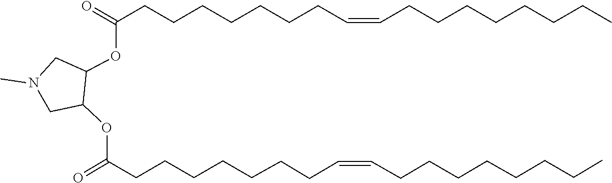 [SMILES: CCCCCCCC/C=C\CCCCCCCC(=O)OC1CN(C)CC1OC(=O)CCCCCCC/C=C\CCCCCCCC] |
| L-10 | 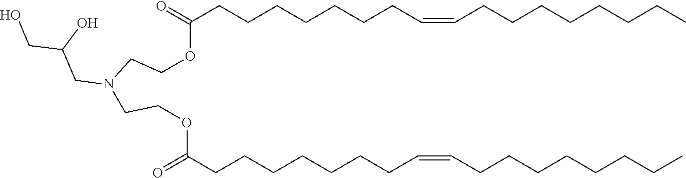 [SMILES: CCCCCCCC/C=C\CCCCCCCC(=O)OCCN(CCOC(=O)CCCCCCC/C=C\CCCCCCCC)CC(O)CO] |

In some embodiments, the ionizable lipid is MC3.

### Series “A”

In some embodiments, an LNP of the present disclosure comprises an ionizable lipid disclosed in PCT Application Publication WO2023044343A1, which is incorporated by reference herein, in its entirety.

### Formula (X)

In some embodiments, ionizable lipids of the present disclosure have a structure of Formula (X):

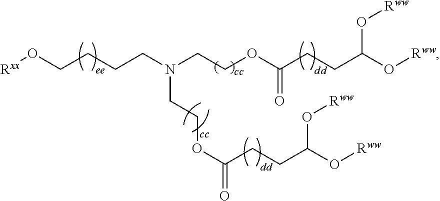

or a pharmaceutically acceptable salt thereof, wherein
each cc is independently selected from 3 to 9;
R^{XX} is selected from hydrogen and optionally substituted C₁-C₆ alkyl; and
(i) ee is 1,
   each dd is independently selected from 1 to 4; and
each R^{WW} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl;
(ii) ee is 0,
   each dd is 1; and
   each R^{WW} is linear C₄-C₁₂ alkyl.

In some embodiments, ionizable lipids of the present disclosure have a structure of Formula (X), wherein R^{XX} is H. In some embodiments, ionizable lipids of the present disclosure have a structure of Formula (X), wherein R^{XX} is optionally substituted C₁-C₆ alkyl. In some embodiments, ionizable lipids of the present disclosure have a structure of Formula (X), wherein R^{XX} is C₁ alkyl. In some embodiments, ionizable lipids of the present disclosure have a structure of Formula (X), wherein R^{XX} is C₂ alkyl. In some embodiments, ionizable lipids of the present disclosure have a structure of Formula (X), wherein R^{XX} is C₃ alkyl. In some embodiments, ionizable lipids of the present disclosure have a structure of Formula (X), wherein R^{XX} is C₄ alkyl. In some embodiments, ionizable lipids of the present disclosure have a structure of Formula (X), wherein R^{XX} is C₅ alkyl. In some embodiments, ionizable lipids of the present disclosure have a structure of Formula (X), wherein R^{XX} is C₆ alkyl.

In some embodiments, ionizable lipids of the present disclosure have a structure of Formula (X), wherein each R^{WW} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl. In some embodiments, ionizable lipids of the present disclosure have a structure of Formula (X), wherein each R^{WW} is C₄-C₁₄ alkyl, wherein any —(CH₂)₂— of the C₄-C₁₄ alkyl can be optionally replaced with C₂-C₆ cycloalkylenyl. In some embodiments, ionizable lipids of the present disclosure have a structure of Formula (X), wherein each R^{WW} is C₄-C₁₄ alkyl, wherein any —(CH₂)₂— of the C₄-C₁₄ alkyl can be optionally replaced with cyclopropylene. In some embodiments, ionizable lipids of the present disclosure have a structure of Formula (X), wherein each R^{WW} is branched C₄-C₁₂ alkenyl. In some embodiments, ionizable lipids of the present disclosure have a structure of Formula (X), wherein each R^{WW} is C₄-C₁₂ alkenyl comprising at least two double bonds. In some embodiments, ionizable lipids of the present disclosure have a structure of Formula (X), wherein each R^{WW} is C₉-C₁₂ alkenyl. In some embodiments, ionizable lipids of the present disclosure have a structure of Formula (X), wherein each R^{WW} is linear C₄-C₁₂ alkyl. In some embodiments, ionizable lipids of the present disclosure have a structure of Formula (X), wherein each R^{WW} is independently selected from the group consisting of C₆-C₁₄ alkyl, branched C₈-C₁₂ alkenyl, C₈-C₁₂ alkenyl comprising at least two double bonds, and C₉-C₁₂ alkenyl, wherein any —(CH₂)₂— of the C₆-C₁₄ alkyl can be optionally replaced with cyclopropylene. In some embodiments, ionizable lipids of the present disclosure have a structure of Formula (X), wherein each R^{WW} is C₆-C₁₄ alkyl, wherein any —(CH₂)₂— of the C₆-C₁₄ alkyl can be optionally replaced with cyclopropylene. In some embodiments, ionizable lipids of the present disclosure have a structure of Formula (X), wherein each R^{WW} is branched C₈-C₁₂ alkenyl, e.g., (linear or branched C₃-C₅ alkylenyl)-(branched C₅-C₇alkenyl), e.g., (branched C₅ alkylenyl)-(branched C₅alkenyl), e.g.,

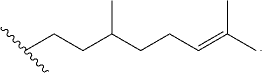 [SMILES: *CCC(C)CCC=C(C)C]

In some embodiments, ionizable lipids of the present disclosure have a structure of Formula (X), wherein each R^{WW} is C₈-C₁₂ alkenyl comprising at least two double bonds. In some embodiments, ionizable lipids of the present disclosure have a structure of Formula (X), wherein each R^{WW} is C₉-C₁₂ alkenyl.

In some embodiments, ionizable lipids of the present disclosure have a structure of Formula (X), wherein each R^{WW} is independently selected from the group consisting of C₆-C₁₄ alkyl (e.g., C₆, C₈, C₉, C₁₀, C₁₁, C₁₃ alkyl), wherein any —(CH₂)₂— of the C₆-C₁₄ alkyl can be optionally replaced with cyclopropylene.

In some embodiments, ionizable lipids of the present disclosure have a structure of Formula (X), wherein each Rww is independently branched C8-C12 alkenyl (e.g., branched C10 alkenyl).

In some embodiments, ionizable lipids of the present disclosure have a structure of Formula (X), wherein each Rww is independently C₈-C₁₂ alkenyl comprising at least two double bonds (e.g., C₉ or C₁₀ alkenyl comprising two double bonds).

In some embodiments, ionizable lipids of the present disclosure have a structure of Formula (X), wherein each Rww is independently (C1 alkylenyl)-(cyclopropylene-C6 alkyl) or (C2 alkylenyl)-(cyclopropylene-C2 alkyl). In some embodiments, ionizable lipids of the present disclosure have a structure of Formula (X), wherein each Rww is independently (C1 alkylenyl)-(cyclopropylene-C6 alkyl). In some embodiments, ionizable lipids of the present disclosure have a structure of Formula (X), wherein each Rww is independently (C₂ alkylenyl)-(cyclopropylene-C₂ alkyl).

In some embodiments, ionizable lipids of the present disclosure have a structure of Formula (X), wherein each Rww is C4 alkyl. In some embodiments, ionizable lipids of the present disclosure have a structure of Formula (X), wherein each Rww is C5 alkyl. In some embodiments, ionizable lipids of the present disclosure have a structure of Formula (X), wherein each Rww is C6 alkyl. In some embodiments, ionizable lipids of the present disclosure have a structure of Formula (X), wherein each Rww is C7 alkyl. In some embodiments, ionizable lipids of the present disclosure have a structure of Formula (X), wherein each Rww is C₈ alkyl. In some embodiments, ionizable lipids of the present disclosure have a structure of Formula (X), wherein each Rww is C9 alkyl. In some embodiments, ionizable lipids of the present disclosure have a structure of Formula (X), wherein each Rww is C10 alkyl. In some embodiments, ionizable lipids of the present disclosure have a structure of Formula (X), wherein each Rww is C11 alkyl. In some embodiments, ionizable lipids of the present disclosure have a structure of Formula (X), wherein each Rww is C12 alkyl. In some embodiments, ionizable lipids of the present disclosure have a structure of Formula (X), wherein each Rww is C13 alkyl. In some embodiments, ionizable lipids of the present disclosure have a structure of Formula (X), wherein each Rww is C14 alkyl.

In some embodiments, ionizable lipids of the present disclosure have a structure of Formula (X), wherein each Rww is C9 alkenyl. In some embodiments, ionizable lipids of the present disclosure have a structure of Formula (X), wherein each Rww is C10 alkenyl. In some embodiments, ionizable lipids of the present disclosure have a structure of Formula (X), wherein each Rww is C11 alkenyl. In some embodiments, ionizable lipids of the present disclosure have a structure of Formula (X), wherein each Rww is C12 alkenyl.

In some embodiments, ionizable lipids of the present disclosure have a structure of Formula (X), wherein each Rww is C8 alkenyl comprising at least two double bonds. In some embodiments, ionizable lipids of the present disclosure have a structure of Formula (X), wherein each Rww is C9 alkenyl comprising at least two double bonds. In some embodiments, ionizable lipids of the present disclosure have a structure of Formula (X), wherein each Rww is C₁₀ alkenyl comprising at least two double bonds. In some embodiments, ionizable lipids of the present disclosure have a structure of Formula (X), wherein each Rww is C11 alkenyl comprising at least two double bonds. In some embodiments, ionizable lipids of the present disclosure have a structure of Formula (X), wherein each Rww is C12 alkenyl comprising at least two double bonds. In some embodiments, ionizable lipids of the present disclosure have a structure of Formula (X), wherein each Rww is C13 alkenyl comprising at least two double bonds. In some embodiments, ionizable lipids of the present disclosure have a structure of Formula (X), wherein each Rww is C14 alkenyl comprising at least two double bonds.

In some embodiments, ionizable lipids of the present disclosure have a structure of Formula (X), wherein each Rww is C9 alkyl, wherein one —(CH₂)₂— of the C₉ alkyl is replaced with C2-C6 cycloalkylenyl. In some embodiments, ionizable lipids of the present disclosure have a structure of Formula (X), wherein each Rww is C9 alkyl, wherein one —(CH2)2- of the C9 alkyl is replaced with cyclopropylene. In some embodiments, ionizable lipids of the present disclosure have a structure of Formula (X), wherein each Rww is C9 alkyl, wherein two —(CH2)2- of the C9 alkyl are replaced with C2-C6 cycloalkylenyl. In some embodiments, ionizable lipids of the present disclosure have a structure of Formula (X), wherein each Rww is C9 alkyl, wherein two —(CH2)2- of the C9 alkyl are replaced with cyclopropylene.

In some embodiments, ionizable lipids of the present disclosure have a structure of Formula (X), wherein each Rww is linear C4 alkyl. In some embodiments, ionizable lipids of the present disclosure have a structure of Formula (X), wherein each Rww is linear C5 alkyl. In some embodiments, ionizable lipids of the present disclosure have a structure of Formula (X), wherein each Rww is linear C6 alkyl. In some embodiments, ionizable lipids of the present disclosure have a structure of Formula (X), wherein each Rww is linear C7 alkyl. In some embodiments, ionizable lipids of the present disclosure have a structure of Formula (X), wherein each Rww is linear C8 alkyl. In some embodiments, ionizable lipids of the present disclosure have a structure of Formula (X), wherein each Rww is linear C9 alkyl. In some embodiments, ionizable lipids of the present disclosure have a structure of Formula (X), wherein each Rww is linear C10 alkyl. In some embodiments, ionizable lipids of the present disclosure have a structure of Formula (X), wherein each Rww is linear C11 alkyl. In some embodiments, ionizable lipids of the present disclosure have a structure of Formula (X), wherein each Rww is linear C12 alkyl. In some embodiments, ionizable lipids of the present disclosure have a structure of Formula (X), wherein each Rww is linear C13 alkyl. In some embodiments, ionizable lipids of the present disclosure have a structure of Formula (X), wherein each Rww is linear C14 alkyl.

In some embodiments, ionizable lipids of the present disclosure have a structure of Formula (X), wherein each Rww is branched C8 alkenyl. In some embodiments, ionizable lipids of the present disclosure have a structure of Formula (X), wherein each Rww is branched C9 alkenyl. In some embodiments, ionizable lipids of the present disclosure have a structure of Formula (X), wherein each Rww is branched C10 alkenyl. In some embodiments, ionizable lipids of the present disclosure have a structure of Formula (X), wherein each Rww is branched C11 alkenyl. In some embodiments, ionizable lipids of the present disclosure have a structure of Formula (X), wherein each Rww is branched C12 alkenyl.

In some embodiments, ionizable lipids of the present disclosure have a structure of Formula (X), wherein each cc is independently selected from 3 to 7. In some embodiments, ionizable lipids of the present disclosure have a structure of Formula (X), wherein each cc is 3. In some embodiments, ionizable lipids of the present disclosure have a structure of Formula (X), wherein each cc is 4. In some embodiments, ionizable lipids of the present disclosure have a structure of Formula (X), wherein each cc is 5. In some embodiments, ionizable lipids of the present disclosure have a structure of Formula (X), wherein each cc is 6. In some embodiments, ionizable lipids of the present disclosure have a structure of Formula (X), wherein each cc is 7. In some embodiments, ionizable lipids of the present disclosure have a structure of Formula (X), wherein each cc is 8. In some embodiments, ionizable lipids of the present disclosure have a structure of Formula (X), wherein each cc is 9.

In some embodiments, ionizable lipids of the present disclosure have a structure of Formula (X), wherein each dd is independently selected from 1 to 4. In some embodiments, ionizable lipids of the present disclosure have a structure of Formula (X), wherein each dd is 1. In some embodiments, ionizable lipids of the present disclosure have a structure of Formula (X), wherein each dd is 2. In some embodiments, ionizable lipids of the present disclosure have a structure of Formula (X), wherein each dd is 3. In some embodiments, ionizable lipids of the present disclosure have a structure of Formula (X), wherein each dd is 4.

In some embodiments, ionizable lipids of the present disclosure have a structure of Formula (X), wherein ee is 1.

In some embodiments, ionizable lipids of the present disclosure have a structure of Formula (X), wherein ee is 0.

### Formula (X-A)

In some embodiments, ionizable lipids of the present disclosure have a structure of Formula (X), wherein the ionizable lipids of the present disclosure have a structure of Formula (X-A):

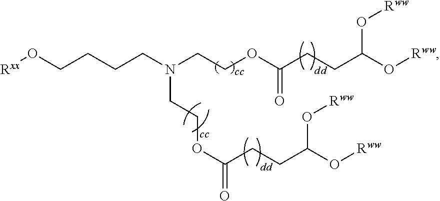

or a pharmaceutically acceptable salt thereof, wherein
each cc is independently selected from 3 to 7;
   each dd is independently selected from 1 to 4;
   R^{XX} is selected from hydrogen and optionally substituted C₁-C₆ alkyl; and
   each R^{ww} is independently selected from the group consisting of C₄-C₁₄ alkyl or (linear or branched C₃-C₅ alkylenyl)-(branched C₅-C₇alkenyl).

In some embodiments, ionizable lipids of the present disclosure have a structure of Formula (X-A), wherein R^{XX} is hydrogen. In some embodiments, ionizable lipids of the present disclosure have a structure of Formula (X-A), wherein R^{XX} is C₁ alkyl. In some embodiments, ionizable lipids of the present disclosure have a structure of Formula (X-A), wherein R^{XX} is C₂ alkyl. In some embodiments, ionizable lipids of the present disclosure have a structure of Formula (X-A), wherein R^{XX} is C₃ alkyl. In some embodiments, ionizable lipids of the present disclosure have a structure of Formula (X-A), wherein R^{XX} is C₄ alkyl. In some embodiments, ionizable lipids of the present disclosure have a structure of Formula (X-A), wherein R^{XX} is C₅ alkyl. In some embodiments, ionizable lipids of the present disclosure have a structure of Formula (X-A), wherein R^{XX} is C₆ alkyl.

In some embodiments, ionizable lipids of the present disclosure have a structure of Formula (X-A), wherein each cc is 4, 5, 6, or 7. In some embodiments, ionizable lipids of the present disclosure have a structure of Formula (X-A), wherein each cc is 3. In some embodiments, ionizable lipids of the present disclosure have a structure of Formula (X-A), wherein each cc is 4. In some embodiments, ionizable lipids of the present disclosure have a structure of Formula (X-A), wherein each cc is 5. In some embodiments, ionizable lipids of the present disclosure have a structure of Formula (X-A), wherein each cc is 6. In some embodiments, ionizable lipids of the present disclosure have a structure of Formula (X-A), wherein each cc is 7.

In some embodiments, ionizable lipids of the present disclosure have a structure of Formula (X-A), wherein each dd is 1 or 3. In some embodiments, ionizable lipids of the present disclosure have a structure of Formula (X-A), wherein each dd is 1. In some embodiments, ionizable lipids of the present disclosure have a structure of Formula (X-A), wherein each dd is 2. In some embodiments, ionizable lipids of the present disclosure have a structure of Formula (X-A), wherein each dd is 3. In some embodiments, ionizable lipids of the present disclosure have a structure of Formula (X-A), wherein each dd is 4.

In some embodiments, ionizable lipids of the present disclosure have a structure of Formula (X-A), wherein each Rww is C4-C₁₄ alkyl. In some embodiments, ionizable lipids of the present disclosure have a structure of Formula (X-A), wherein each Rww is C4 alkyl. In some embodiments, ionizable lipids of the present disclosure have a structure of Formula (X-A), wherein each Rww is C5 alkyl. In some embodiments, ionizable lipids of the present disclosure have a structure of Formula (X-A), wherein each Rww is C6 alkyl. In some embodiments, ionizable lipids of the present disclosure have a structure of Formula (X-A), wherein each Rww is C7 alkyl. In some embodiments, ionizable lipids of the present disclosure have a structure of Formula (X-A), wherein each Rww is C8 alkyl. In some embodiments, ionizable lipids of the present disclosure have a structure of Formula (X-A), wherein each Rww is C₉ alkyl. In some embodiments, ionizable lipids of the present disclosure have a structure of Formula (X-A), wherein each Rww is C10 alkyl. In some embodiments, ionizable lipids of the present disclosure have a structure of Formula (X-A), wherein each Rww is C11 alkyl. In some embodiments, ionizable lipids of the present disclosure have a structure of Formula (X-A), wherein each Rww is C12 alkyl. In some embodiments, ionizable lipids of the present disclosure have a structure of Formula (X-A), wherein each Rww is C13 alkyl. In some embodiments, ionizable lipids of the present disclosure have a structure of Formula (X-A), wherein each Rww is C14 alkyl.

In some embodiments, ionizable lipids of the present disclosure have a structure of Formula (X-A), wherein each R^{WW} is (linear or branched C₃-C₅ alkylenyl)-(branched C₅-C₇alkenyl), e.g., (branched C₅ alkylenyl)-(branched C₅alkenyl), e.g.,

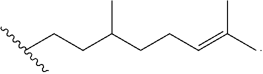 [SMILES: *CCC(C)CCC=C(C)C]

In some embodiments, ionizable lipids of the present disclosure comprise an acyclic core. In some embodiments, ionizable lipids of the present disclosure are selected from any lipid in Table (I-A) below or a pharmaceutically acceptable salt thereof:

TABLE (I-A)
Non-Limiting Examples of Ionizable Lipids with an Acyclic Core

| Structure | # |
|---|---|
| 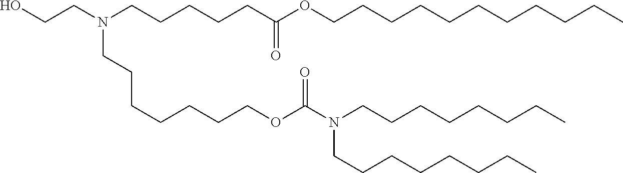 [SMILES: CCCCCCCCCCCOC(=O)CCCCCN(CCO)CCCCCCCOC(=O)N(CCCCCCCC)CCCCCCCC] | 1 |
| 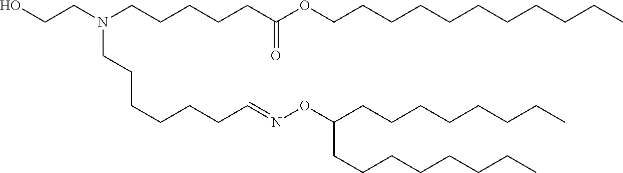 [SMILES: CCCCCCCCCCCOC(=O)CCCCCN(CCO)CCCCCC/C=N/OC(CCCCCCCC)CCCCCCCC] | 2 |
| 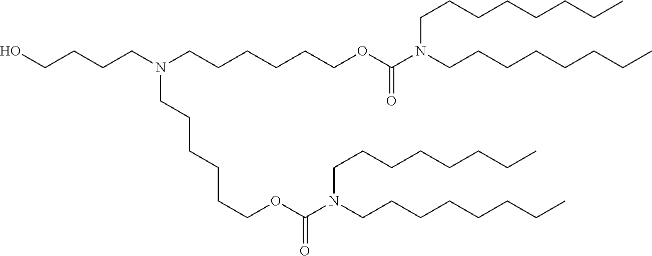 [SMILES: CCCCCCCCN(CCCCCCCC)C(=O)OCCCCCCN(CCCCO)CCCCCCOC(=O)N(CCCCCCCC)CCCCCCCC] | 3 |
| 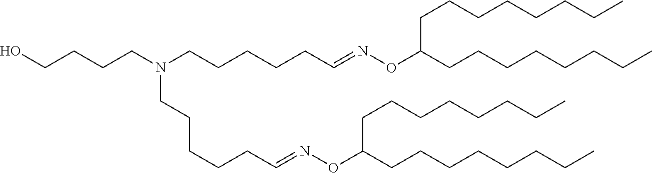 [SMILES: CCCCCCCCC(CCCCCCCC)O/N=C/CCCCCN(CCCCO)CCCCC/C=N/OC(CCCCCCCC)CCCCCCCC] | 4 |
| 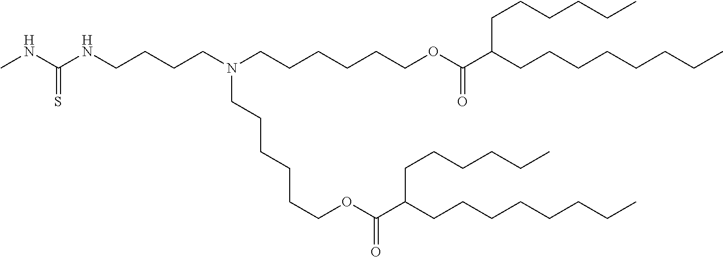 [SMILES: CCCCCCCCC(CCCCCC)C(=O)OCCCCCCN(CCCCCCOC(=O)C(CCCCCC)CCCCCCCC)CCCCNC(=S)NC] | 5 |
| 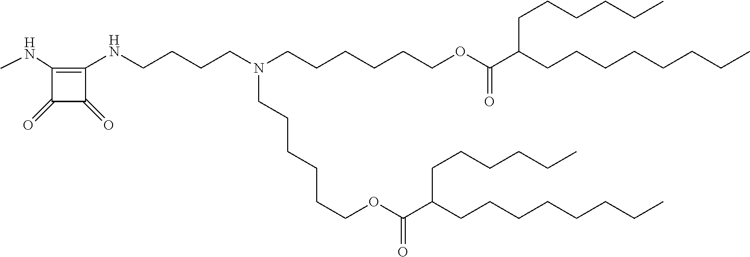 [SMILES: CCCCCCCCC(CCCCCC)C(=O)OCCCCCCN(CCCCCCOC(=O)C(CCCCCC)CCCCCCCC)CCCCNc1c(NC)c(=O)c1=O] | 6 |
| 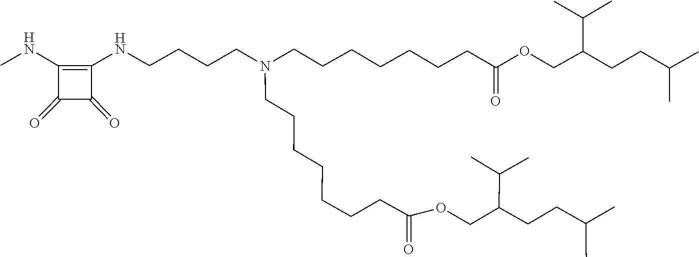 [SMILES: CNc1c(NCCCCN(CCCCCCCC(=O)OCC(CCC(C)C)C(C)C)CCCCCCCC(=O)OCC(CCC(C)C)C(C)C)c(=O)c1=O] | 7 |
| 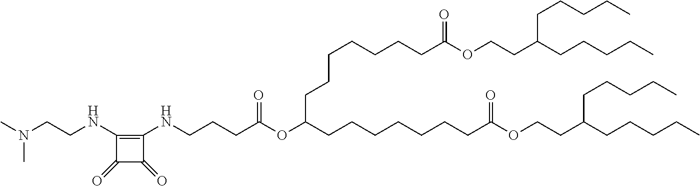 [SMILES: CCCCCC(CCCCC)CCOC(=O)CCCCCCCC(CCCCCCCC(=O)OCCC(CCCCC)CCCCC)OC(=O)CCCNc1c(NCCN(C)C)c(=O)c1=O] | 8 |
| 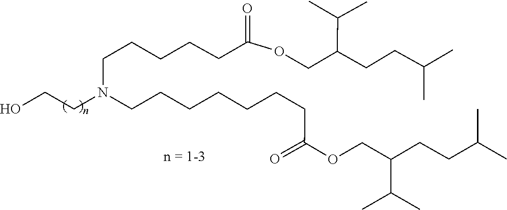 | 9 |
| 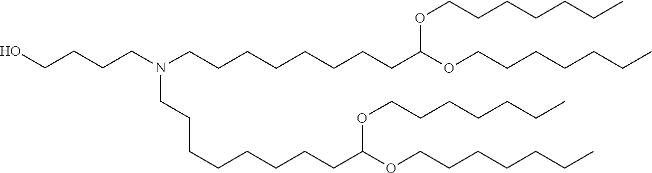 [SMILES: CCCCCCCOC(CCCCCCCCN(CCCCO)CCCCCCCCC(OCCCCCCC)OCCCCCCC)OCCCCCCC] | 10 |
| 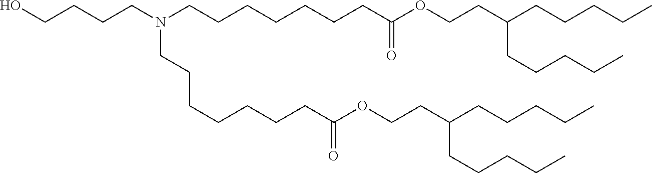 [SMILES: CCCCCC(CCCCC)CCOC(=O)CCCCCCCN(CCCCO)CCCCCCCC(=O)OCCC(CCCCC)CCCCC] | 11 |
| 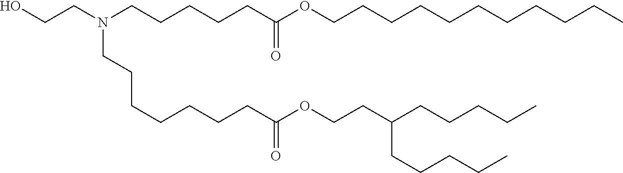 [SMILES: CCCCCCCCCCCOC(=O)CCCCCN(CCO)CCCCCCCC(=O)OCCC(CCCCC)CCCCC] | 13 |
| 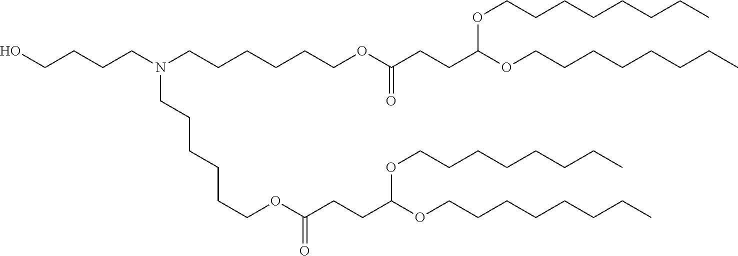 [SMILES: CCCCCCCCOC(CCC(=O)OCCCCCCN(CCCCO)CCCCCCOC(=O)CCC(OCCCCCCCC)OCCCCCCCC)OCCCCCCCC] | 14 |
| 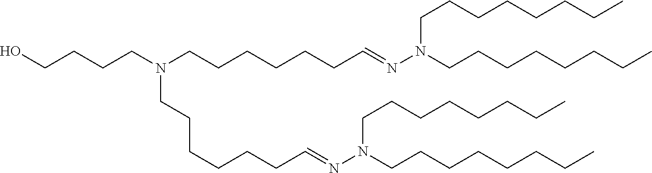 [SMILES: CCCCCCCCN(CCCCCCCC)/N=C/CCCCCCN(CCCCO)CCCCCC/C=N/N(CCCCCCCC)CCCCCCCC] | 15 |
| 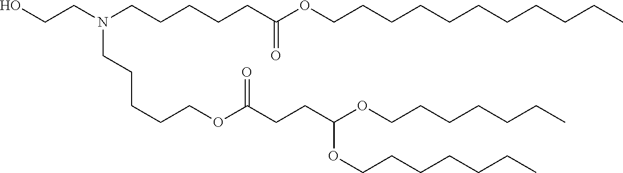 [SMILES: CCCCCCCCCCCOC(=O)CCCCCN(CCO)CCCCCOC(=O)CCC(OCCCCCCC)OCCCCCCC] | 16 |
| 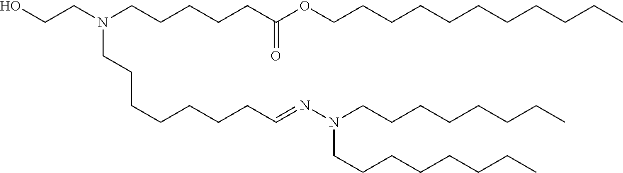 [SMILES: CCCCCCCCCCCOC(=O)CCCCCN(CCO)CCCCCCC/C=N/N(CCCCCCCC)CCCCCCCC] | 17 |
| 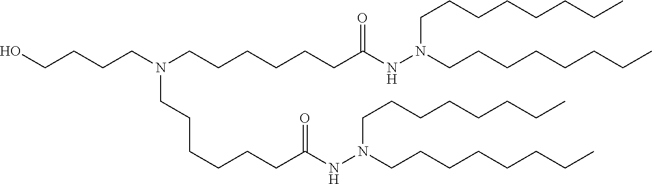 [SMILES: CCCCCCCCN(CCCCCCCC)NC(=O)CCCCCCN(CCCCO)CCCCCCC(=O)NN(CCCCCCCC)CCCCCCCC] | 18 |
| 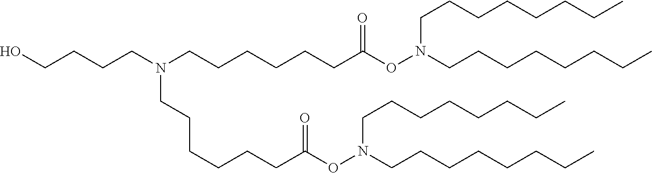 [SMILES: CCCCCCCCN(CCCCCCCC)OC(=O)CCCCCCN(CCCCO)CCCCCCC(=O)ON(CCCCCCCC)CCCCCCCC] | 19 |
| 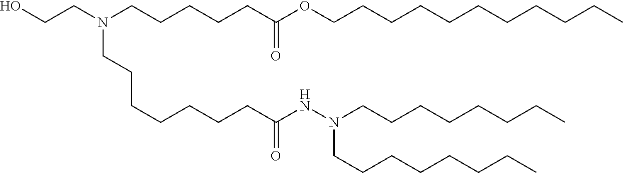 [SMILES: CCCCCCCCCCCOC(=O)CCCCCN(CCO)CCCCCCCC(=O)NN(CCCCCCCC)CCCCCCCC] | 20 |
| 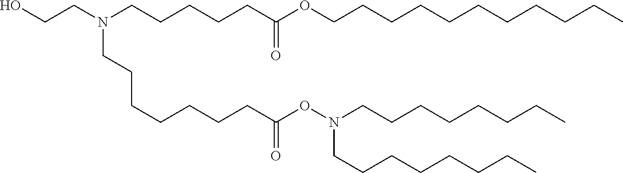 [SMILES: CCCCCCCCCCCOC(=O)CCCCCN(CCO)CCCCCCCC(=O)ON(CCCCCCCC)CCCCCCCC] | 21 |
| 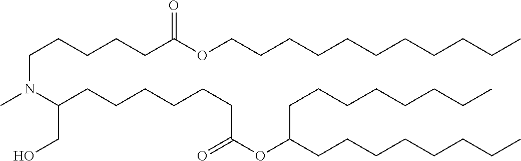 [SMILES: CCCCCCCCCCCOC(=O)CCCCCN(C)C(CO)CCCCCCC(=O)OC(CCCCCCCC)CCCCCCCC] | 22 |
| 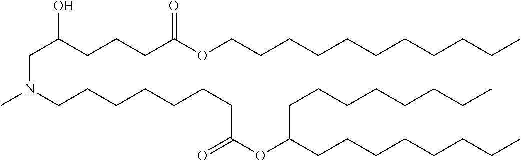 [SMILES: CCCCCCCCCCCOC(=O)CCCC(O)CN(C)CCCCCCCC(=O)OC(CCCCCCCC)CCCCCCCC] | 23 |
| 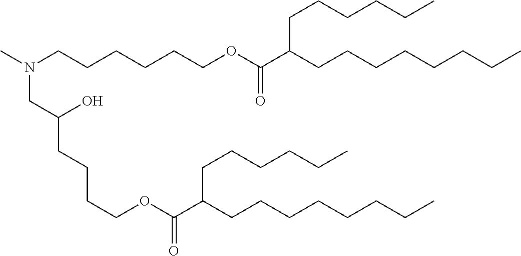 [SMILES: CCCCCCCCC(CCCCCC)C(=O)OCCCCCCN(C)CC(O)CCCCOC(=O)C(CCCCCC)CCCCCCCC] | 24 |
| 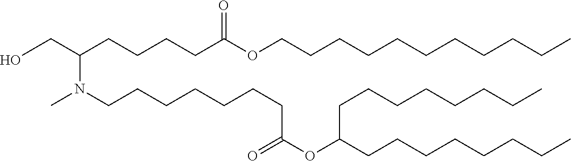 | 25 |
| 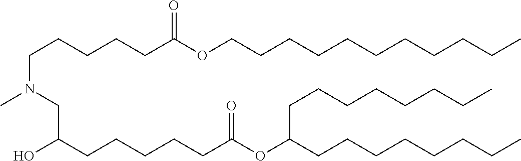 [SMILES: CCCCCCCCCCCOC(=O)CCCCCN(C)CC(O)CCCCCC(=O)OC(CCCCCCCC)CCCCCCCC] | 26 |
| 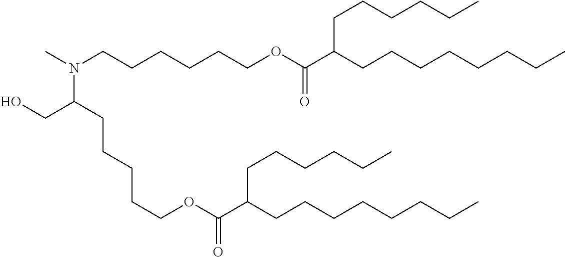 [SMILES: CCCCCCCCC(CCCCCC)C(=O)OCCCCCCN(C)C(CO)CCCCCOC(=O)C(CCCCCC)CCCCCCCC] | 27 |
| 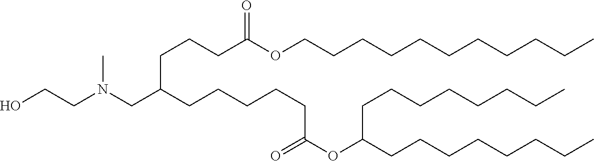 [SMILES: CCCCCCCCCCCOC(=O)CCCC(CCCCCC(=O)OC(CCCCCCCC)CCCCCCCC)CN(C)CCO] | 28 |
| 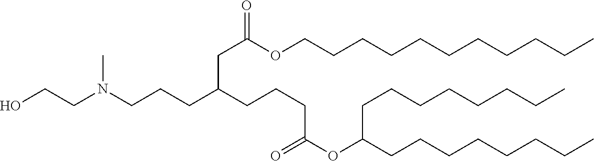 [SMILES: CCCCCCCCCCCOC(=O)CC(CCCC(=O)OC(CCCCCCCC)CCCCCCCC)CCCN(C)CCO] | 29 |
| 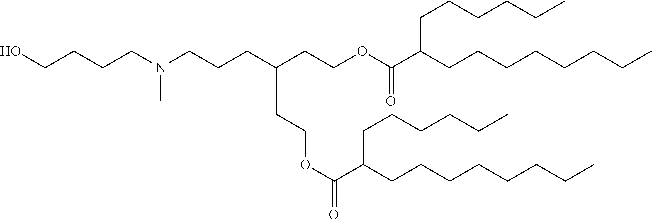 [SMILES: CCCCCCCCC(CCCCCC)C(=O)OCCC(CCCN(C)CCCCO)CCOC(=O)C(CCCCCC)CCCCCCCC] | 30 |
| 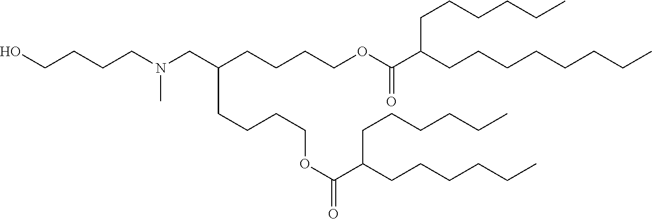 [SMILES: CCCCCCCCC(CCCCCC)C(=O)OCCCCC(CCCCOC(=O)C(CCCCCC)CCCCCC)CN(C)CCCCO] | 31 |
| 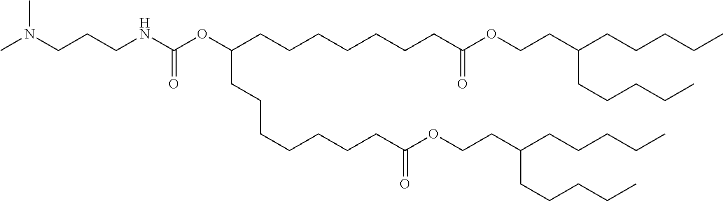 [SMILES: CCCCCC(CCCCC)CCOC(=O)CCCCCCCC(CCCCCCCC(=O)OCCC(CCCCC)CCCCC)OC(=O)NCCCN(C)C] | 32 |
| 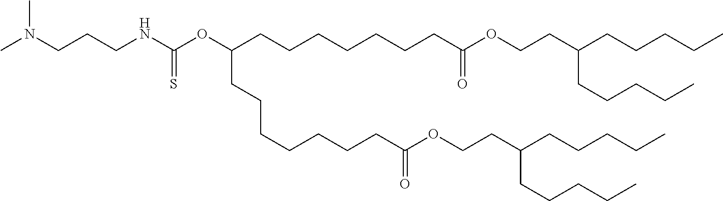 [SMILES: CCCCCC(CCCCC)CCOC(=O)CCCCCCCC(CCCCCCCC(=O)OCCC(CCCCC)CCCCC)OC(=S)NCCCN(C)C] | 33 |
| 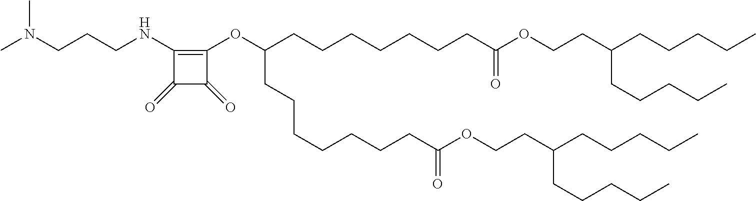 [SMILES: CCCCCC(CCCCC)CCOC(=O)CCCCCCCC(CCCCCCCC(=O)OCCC(CCCCC)CCCCC)Oc1c(NCCCN(C)C)c(=O)c1=O] | 34 |
| 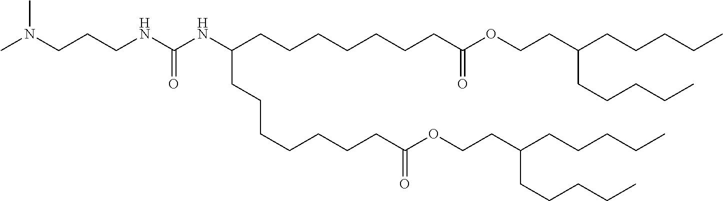 [SMILES: CCCCCC(CCCCC)CCOC(=O)CCCCCCCC(CCCCCCCC(=O)OCCC(CCCCC)CCCCC)NC(=O)NCCCN(C)C] | 35 |
| 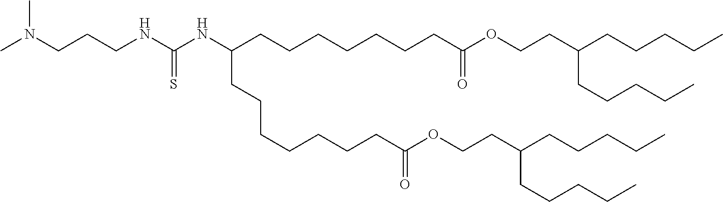 [SMILES: CCCCCC(CCCCC)CCOC(=O)CCCCCCCC(CCCCCCCC(=O)OCCC(CCCCC)CCCCC)NC(=S)NCCCN(C)C] | 36 |
| 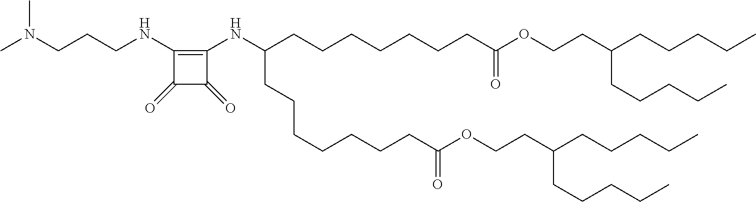 [SMILES: CCCCCC(CCCCC)CCOC(=O)CCCCCCCC(CCCCCCCC(=O)OCCC(CCCCC)CCCCC)Nc1c(NCCCN(C)C)c(=O)c1=O] | 37 |
| 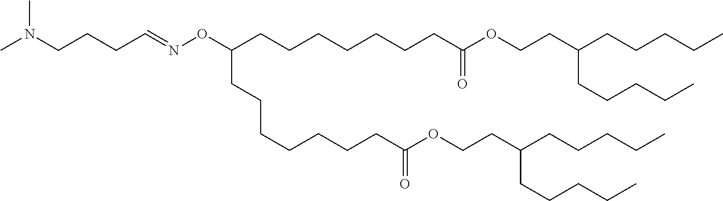 [SMILES: CCCCCC(CCCCC)CCOC(=O)CCCCCCCC(CCCCCCCC(=O)OCCC(CCCCC)CCCCC)O/N=C/CCCN(C)C] | 38 |
| 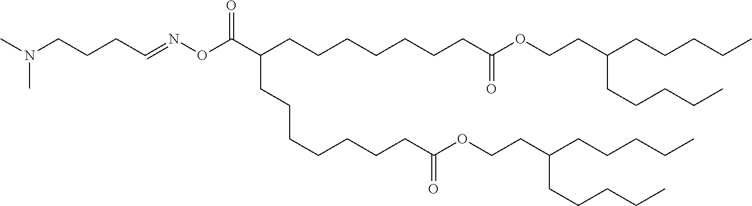 [SMILES: CCCCCC(CCCCC)CCOC(=O)CCCCCCCC(CCCCCCCC(=O)OCCC(CCCCC)CCCCC)C(=O)O/N=C/CCCN(C)C] | 39 |
| 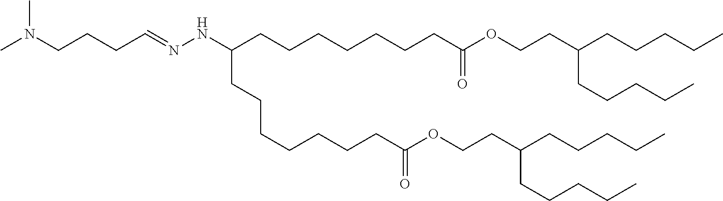 [SMILES: CCCCCC(CCCCC)CCOC(=O)CCCCCCCC(CCCCCCCC(=O)OCCC(CCCCC)CCCCC)N/N=C/CCCN(C)C] | 40 |
| 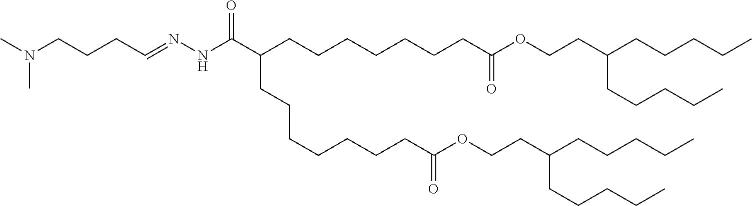 [SMILES: CCCCCC(CCCCC)CCOC(=O)CCCCCCCC(CCCCCCCC(=O)OCCC(CCCCC)CCCCC)C(=O)N/N=C/CCCN(C)C] | 41 |
| 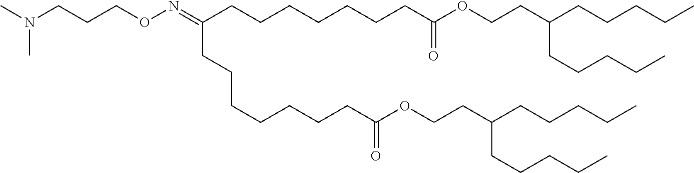 [SMILES: CCCCCC(CCCCC)CCOC(=O)CCCCCCCC(CCCCCCCC(=O)OCCC(CCCCC)CCCCC)=NOCCCN(C)C] | 42 |
| 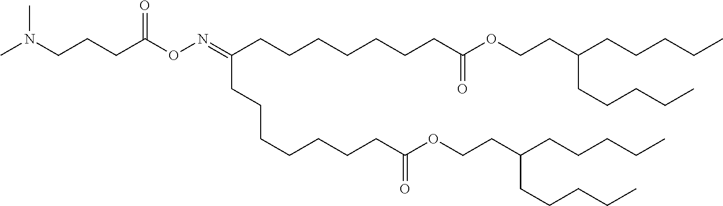 [SMILES: CCCCCC(CCCCC)CCOC(=O)CCCCCCCC(CCCCCCCC(=O)OCCC(CCCCC)CCCCC)=NOC(=O)CCCN(C)C] | 43 |
| 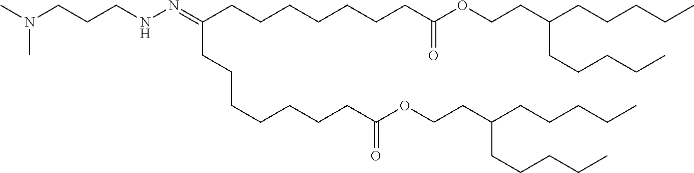 [SMILES: CCCCCC(CCCCC)CCOC(=O)CCCCCCCC(CCCCCCCC(=O)OCCC(CCCCC)CCCCC)=NNCCCN(C)C] | 44 |
| 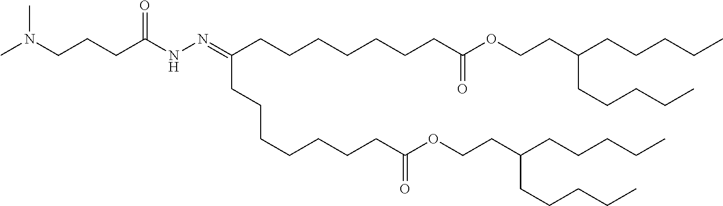 [SMILES: CCCCCC(CCCCC)CCOC(=O)CCCCCCCC(CCCCCCCC(=O)OCCC(CCCCC)CCCCC)=NNC(=O)CCCN(C)C] | 45 |
| 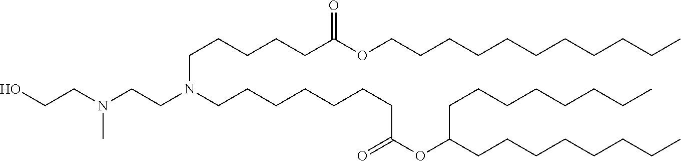 [SMILES: CCCCCCCCCCCOC(=O)CCCCCN(CCCCCCCC(=O)OC(CCCCCCCC)CCCCCCCC)CCN(C)CCO] | 46 |
| 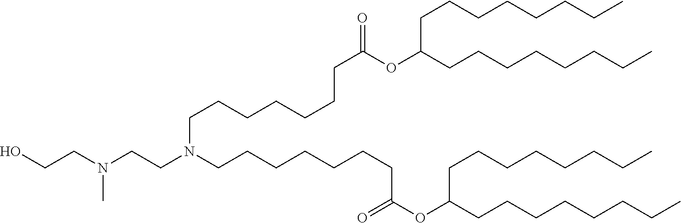 [SMILES: CCCCCCCCC(CCCCCCCC)OC(=O)CCCCCCCN(CCCCCCCC(=O)OC(CCCCCCCC)CCCCCCCC)CCN(C)CCO] | 47 |
| 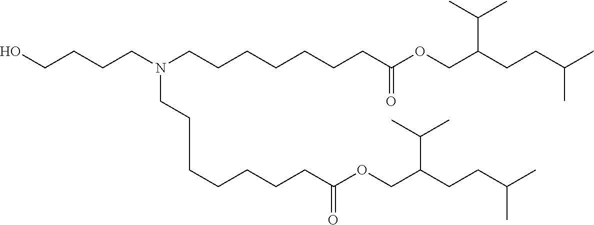 [SMILES: CC(C)CCC(COC(=O)CCCCCCCN(CCCCO)CCCCCCCC(=O)OCC(CCC(C)C)C(C)C)C(C)C] | 48 |
| 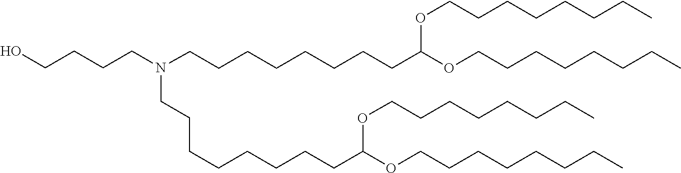 [SMILES: CCCCCCCCOC(CCCCCCCCN(CCCCO)CCCCCCCCC(OCCCCCCCC)OCCCCCCCC)OCCCCCCCC] | 49 |
| 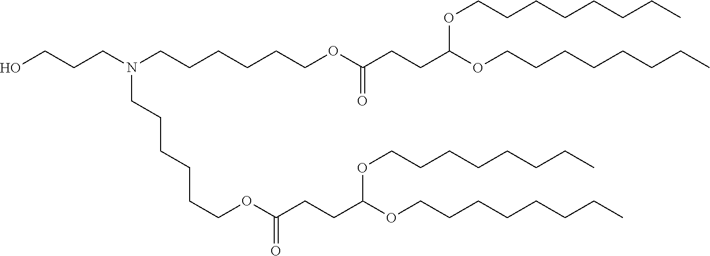 [SMILES: CCCCCCCCOC(CCC(=O)OCCCCCCN(CCCO)CCCCCCOC(=O)CCC(OCCCCCCCC)OCCCCCCCC)OCCCCCCCC] | 50 |
| 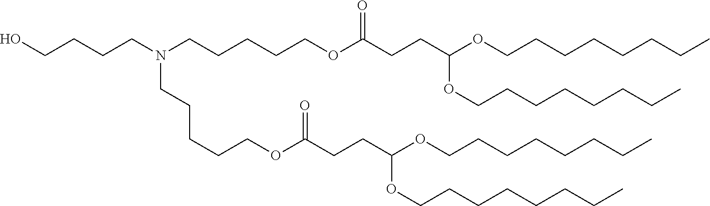 [SMILES: CCCCCCCCOC(CCC(=O)OCCCCCN(CCCCO)CCCCCOC(=O)CCC(OCCCCCCCC)OCCCCCCCC)OCCCCCCCC] | 51 |
| 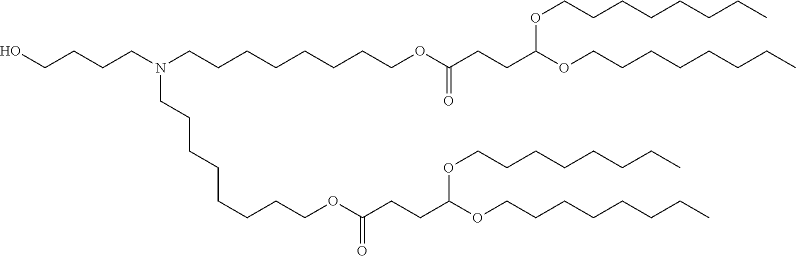 [SMILES: CCCCCCCCOC(CCC(=O)OCCCCCCCCN(CCCCO)CCCCCCCCOC(=O)CCC(OCCCCCCCC)OCCCCCCCC)OCCCCCCCC] | 52 |
| 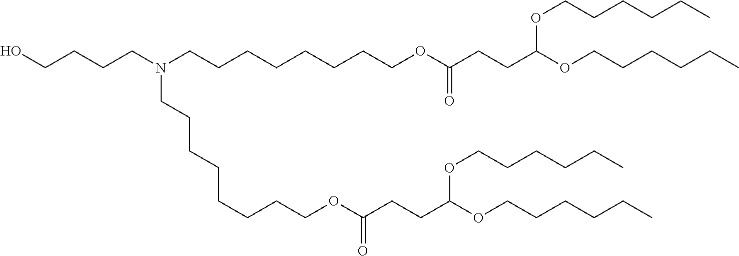 [SMILES: CCCCCCOC(CCC(=O)OCCCCCCCCN(CCCCO)CCCCCCCCOC(=O)CCC(OCCCCCC)OCCCCCC)OCCCCCC] | 53 |
| 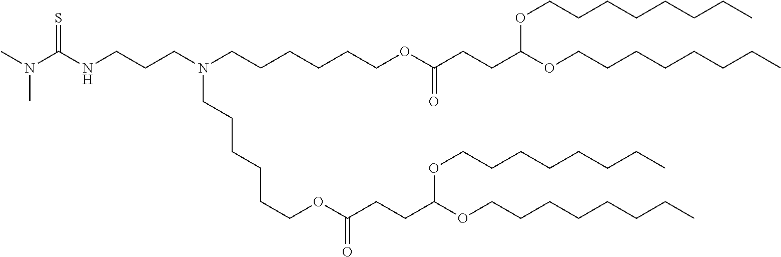 [SMILES: CCCCCCCCOC(CCC(=O)OCCCCCCN(CCCCCCOC(=O)CCC(OCCCCCCCC)OCCCCCCCC)CCCNC(=S)N(C)C)OCCCCCCCC] | 54 |
| 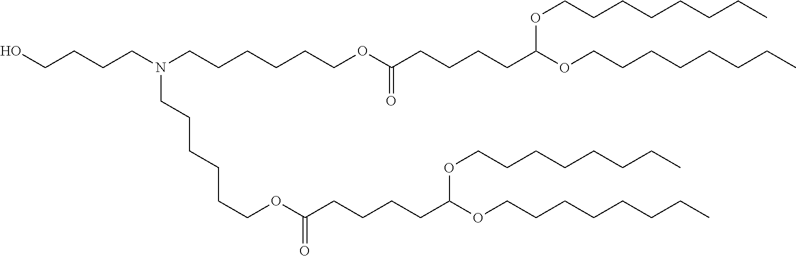 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 55 |
| 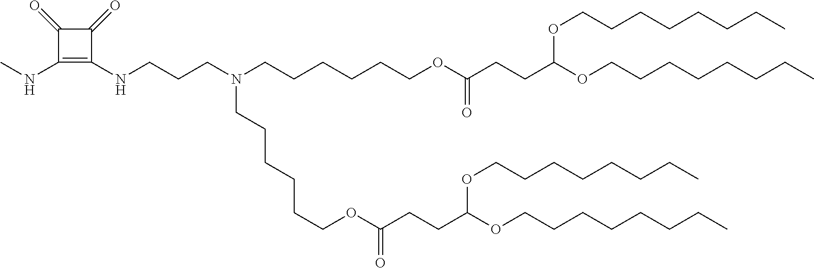 [SMILES: CCCCCCCCOC(CCC(=O)OCCCCCCN(CCCCCCOC(=O)CCC(OCCCCCCCC)OCCCCCCCC)CCCNc1c(NC)c(=O)c1=O)OCCCCCCCC] | 56 |
| 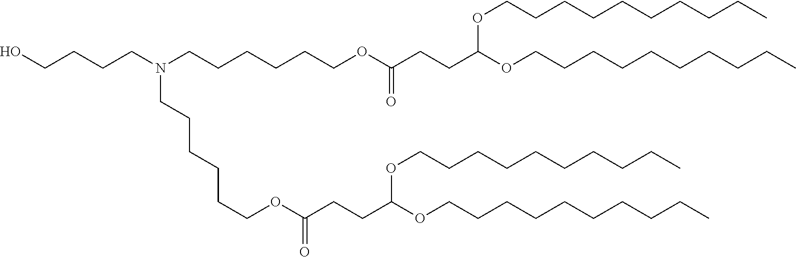 [SMILES: CCCCCCCCCCOC(CCC(=O)OCCCCCCN(CCCCO)CCCCCCOC(=O)CCC(OCCCCCCCCCC)OCCCCCCCCCC)OCCCCCCCCCC] | 57 |
| 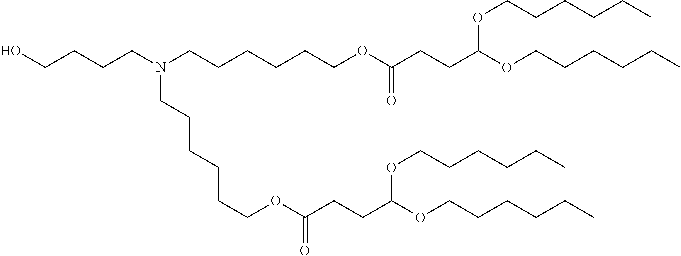 [SMILES: CCCCCCOC(CCC(=O)OCCCCCCN(CCCCO)CCCCCCOC(=O)CCC(OCCCCCC)OCCCCCC)OCCCCCC] | 58 |
| 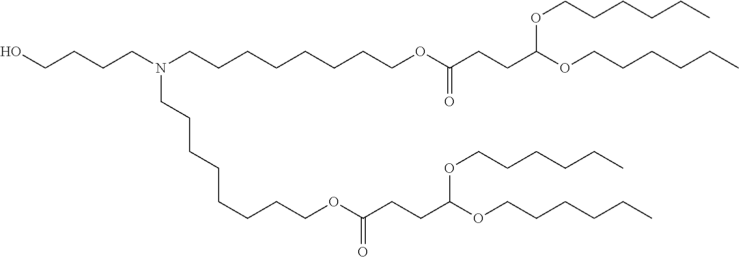 [SMILES: CCCCCCOC(CCC(=O)OCCCCCCCCN(CCCCO)CCCCCCCCOC(=O)CCC(OCCCCCC)OCCCCCC)OCCCCCC] | 59 |
| 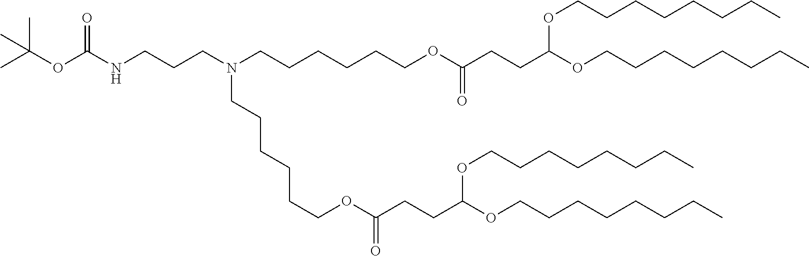 [SMILES: CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC] | 60 |
| 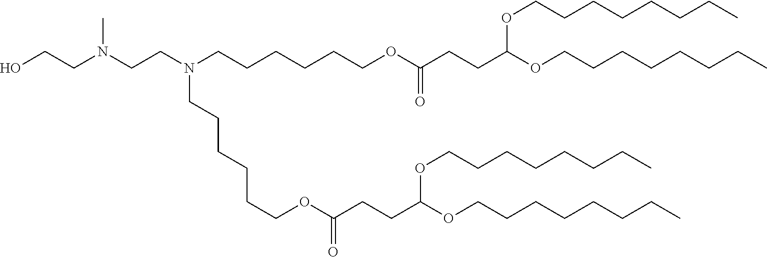 [SMILES: CCCCCCCCOC(CCC(=O)OCCCCCCN(CCCCCCOC(=O)CCC(OCCCCCCCC)OCCCCCCCC)CCN(C)CCO)OCCCCCCCC] | 61 |
| 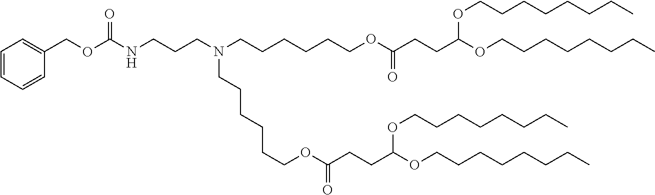 [SMILES: CCCCCCCCOC(CCC(=O)OCCCCCCN(CCCCCCOC(=O)CCC(OCCCCCCCC)OCCCCCCCC)CCCNC(=O)OCc1ccccc1)OCCCCCCCC] | 62 |
| 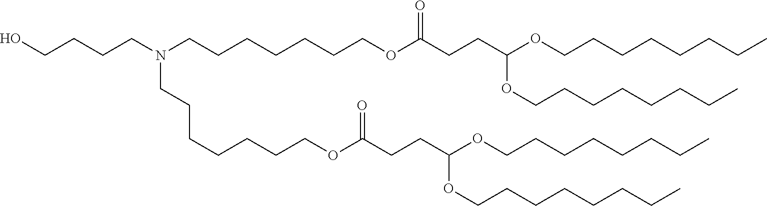 [SMILES: CCCCCCCCOC(CCC(=O)OCCCCCCCN(CCCCO)CCCCCCCOC(=O)CCC(OCCCCCCCC)OCCCCCCCC)OCCCCCCCC] | 64 |
| 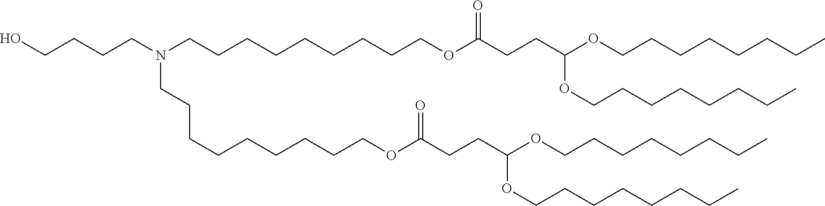 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC] | 65 |
| 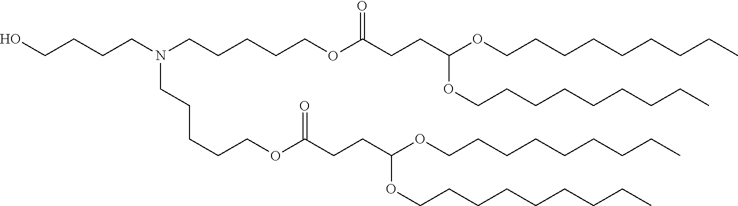 [SMILES: CCCCCCCCCOC(CCC(=O)OCCCCCN(CCCCO)CCCCCOC(=O)CCC(OCCCCCCCCC)OCCCCCCCCC)OCCCCCCCCC] | 66 |
| 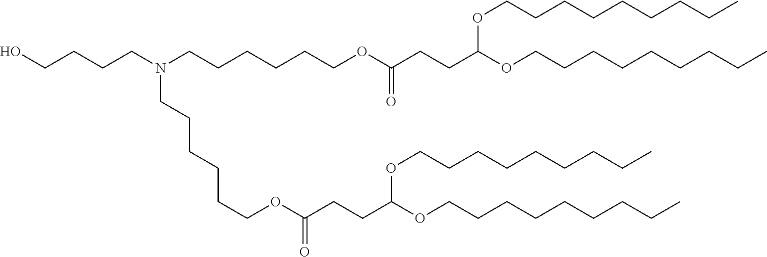 [SMILES: CCCCCCCCCOC(CCC(=O)OCCCCCCN(CCCCO)CCCCCCOC(=O)CCC(OCCCCCCCCC)OCCCCCCCCC)OCCCCCCCCC] | 67 |
| 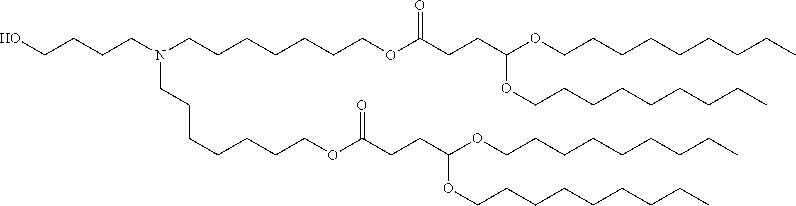 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC] | 68 |
| 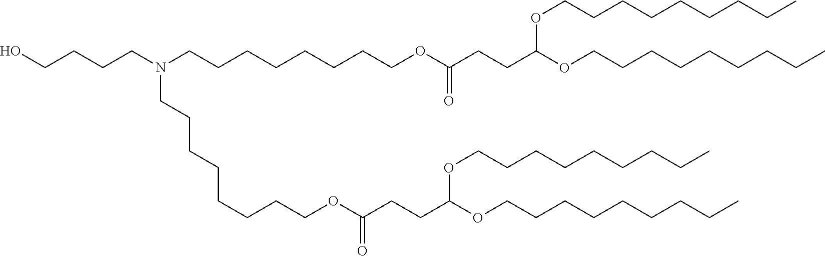 [SMILES: CCCCCCCCCOC(CCC(=O)OCCCCCCCCN(CCCCO)CCCCCCCCOC(=O)CCC(OCCCCCCCCC)OCCCCCCCCC)OCCCCCCCCC] | 69 |
| 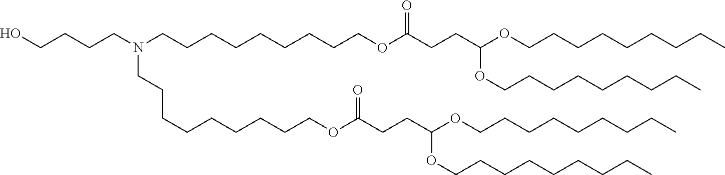 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC] | 70 |
| 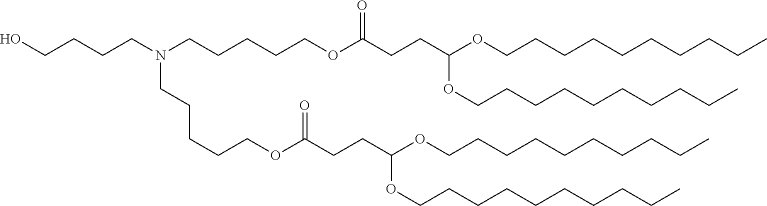 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC] | 71 |
| 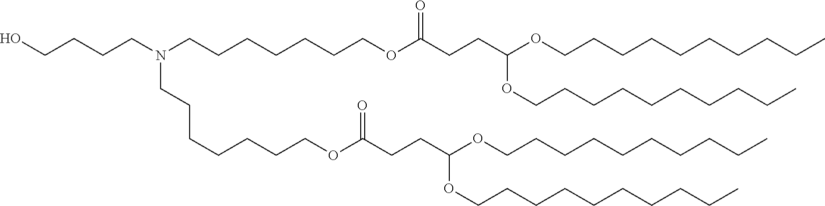 [SMILES: CCCCCCCCCCOC(CCC(=O)OCCCCCCCN(CCCCO)CCCCCCCOC(=O)CCC(OCCCCCCCCCC)OCCCCCCCCCC)OCCCCCCCCCC] | 72 |
| 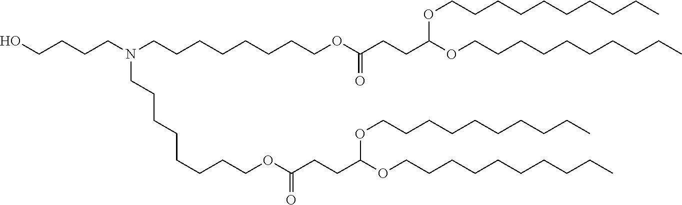 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC] | 73 |
| 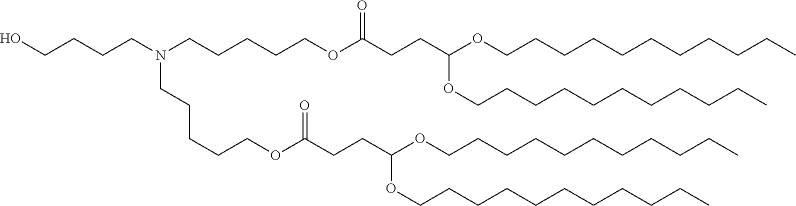 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC] | 74 |
| 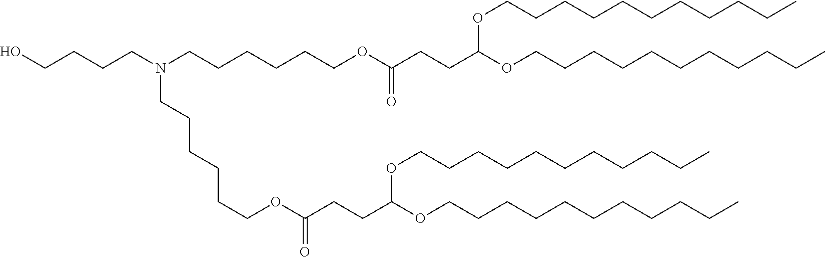 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC] | 75 |
| 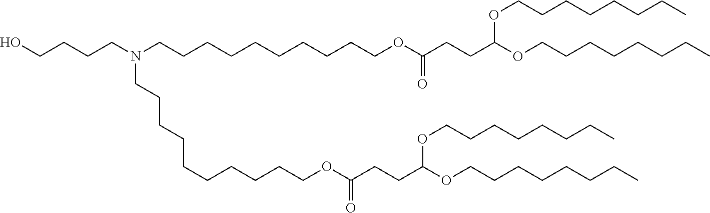 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC] | 76 |
| 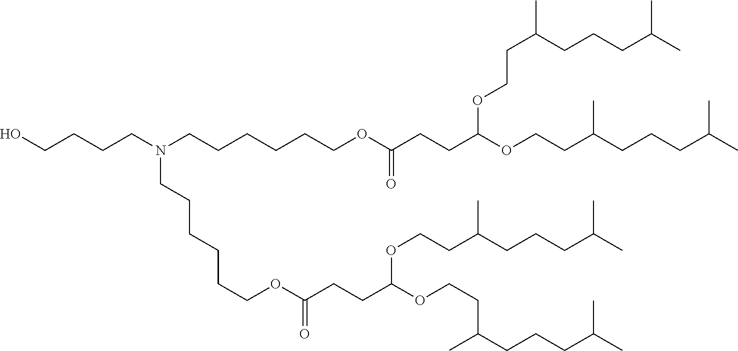 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C] | 77 |
| 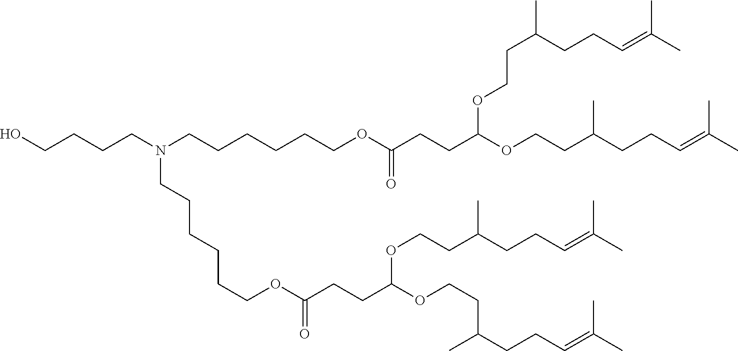 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C] | 78 |
| 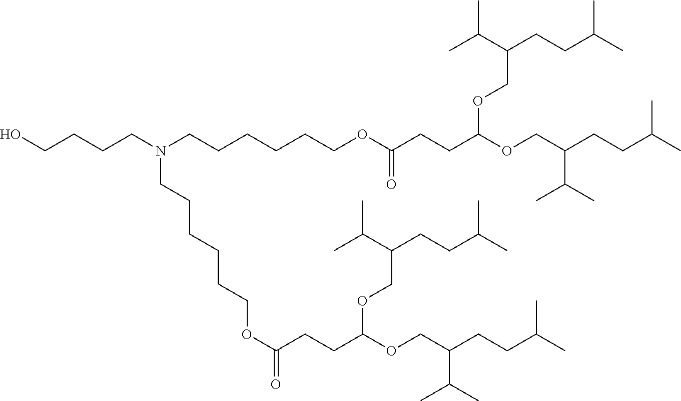 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C] | 79 |
| 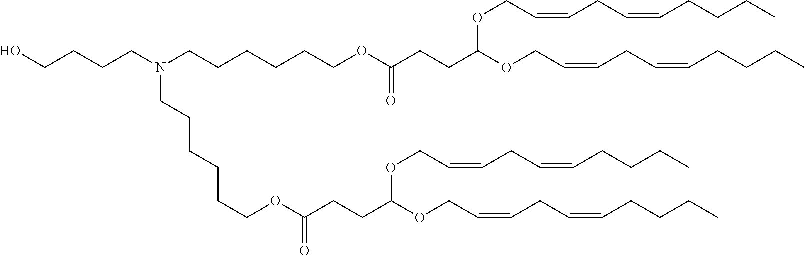 [SMILES: CCCC/C=C\C/C=C\COC(CCC(=O)OCCCCCCN(CCCCO)CCCCCCOC(=O)CCC(OC/C=C\C/C=C\CCCC)OC/C=C\C/C=C\CCCC)OC/C=C\C/C=C\CCCC] | 80 |
| 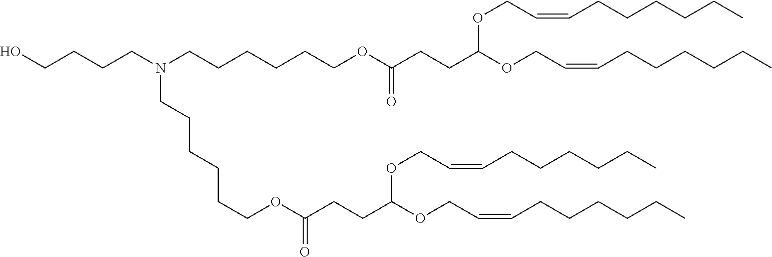 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC] | 81 |
| 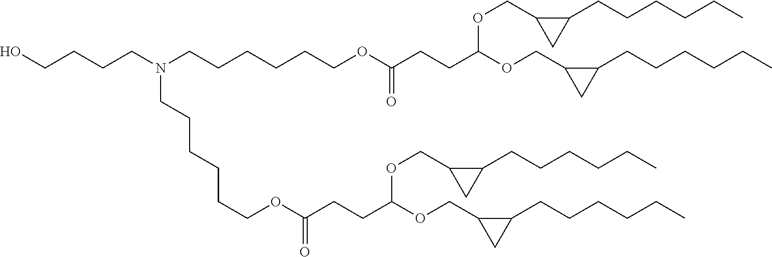 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC] | 82 |
| 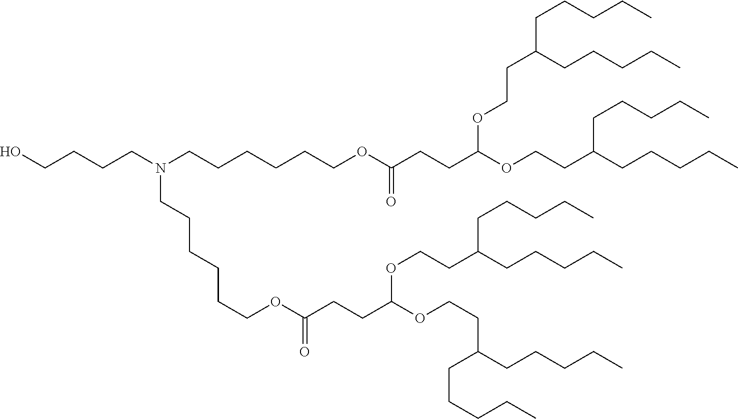 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC] | 83 |
| 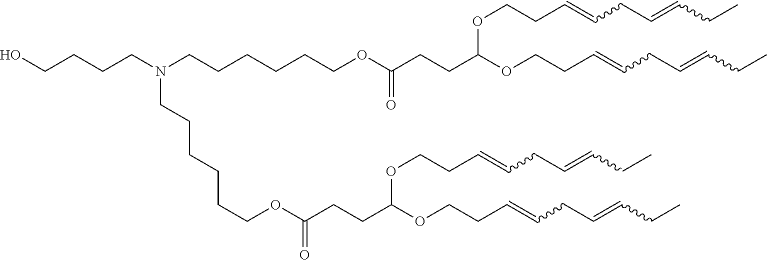 [SMILES: CCC=CCC=CCCOC(CCC(=O)OCCCCCCN(CCCCO)CCCCCCOC(=O)CCC(OCCC=CCC=CCC)OCCC=CCC=CCC)OCCC=CCC=CCC] | 84 |
| 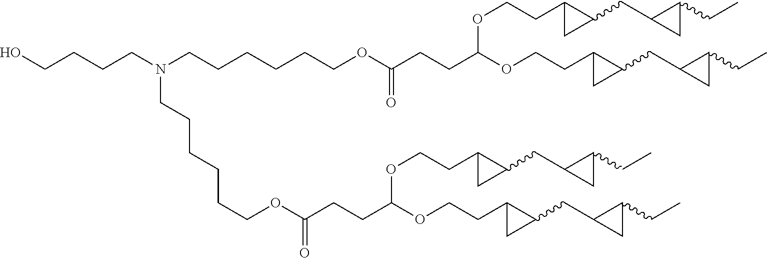 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC] | 85 |
| 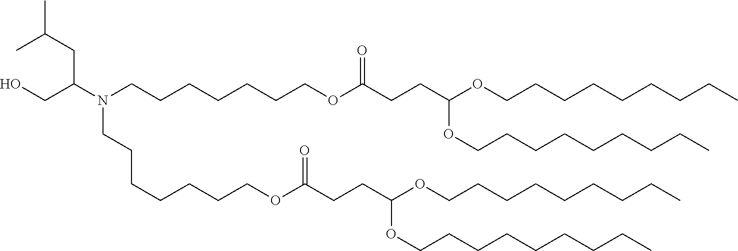 [SMILES: CCCCCCCCCOC(CCC(=O)OCCCCCCCN(CCCCCCCOC(=O)CCC(OCCCCCCCCC)OCCCCCCCCC)C(CO)CC(C)C)OCCCCCCCCC] | 86 |
| 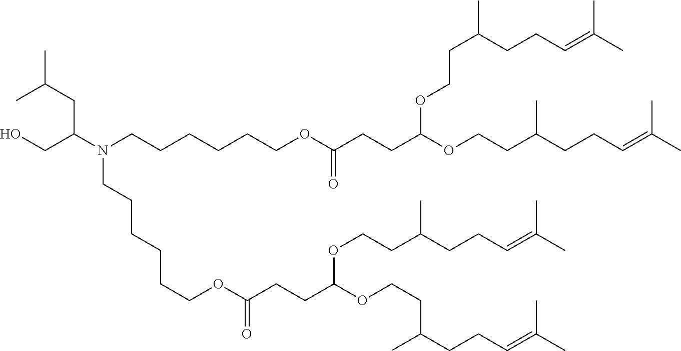 [SMILES: CC(C)=CCCC(C)CCOC(CCC(=O)OCCCCCCN(CCCCCCOC(=O)CCC(OCCC(C)CCC=C(C)C)OCCC(C)CCC=C(C)C)C(CO)CC(C)C)OCCC(C)CCC=C(C)C] | 87 |
| 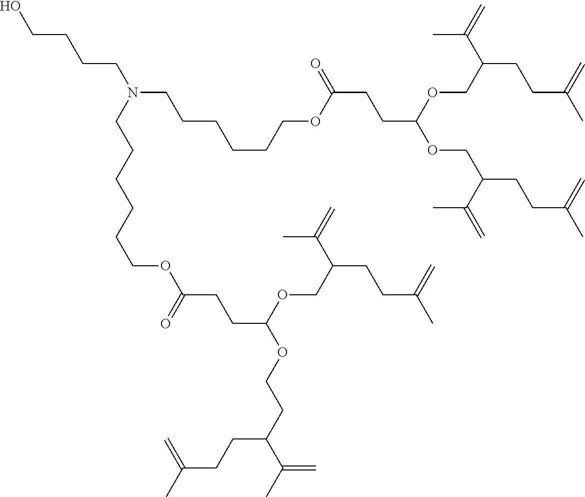 [SMILES: C=C(C)CCC(CCOC(CCC(=O)OCCCCCCN(CCCCO)CCCCCCOC(=O)CCC(OCC(CCC(=C)C)C(=C)C)OCC(CCC(=C)C)C(=C)C)OCC(CCC(=C)C)C(=C)C)C(=C)C] | 88 |
| 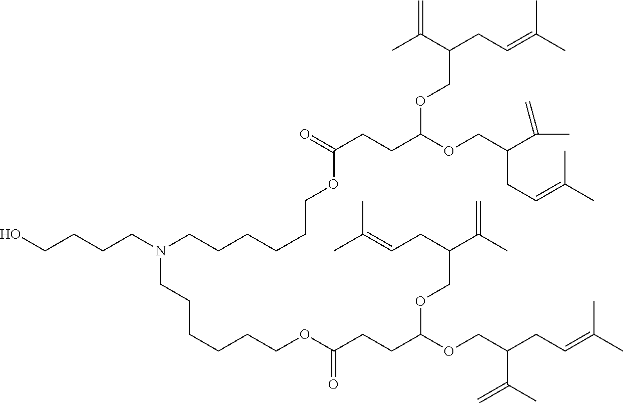 [SMILES: C=C(C)C(CC=C(C)C)COC(CCC(=O)OCCCCCCN(CCCCO)CCCCCCOC(=O)CCC(OCC(CC=C(C)C)C(=C)C)OCC(CC=C(C)C)C(=C)C)OCC(CC=C(C)C)C(=C)C] | 89 |

### Series “CY”

In some embodiments, an LNP of the present disclosure comprises an ionizable lipid disclosed in PCT Application Publication WO2023044333A1, which is incorporated by reference herein, in its entirety.

### Formula (CY)

In some embodiments, an LNP disclosed herein comprises an ionizable lipid of Formula (CY)

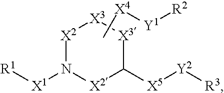

or a pharmaceutically acceptable salt thereof, wherein:
R¹ is selected from the group consisting of —OH, —OAc, R^{1a},

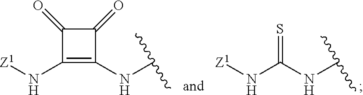

Z¹ is optionally substituted C₁-C₆ alkyl;
X¹ is optionally substituted C₂-C₆ alkylenyl;
X² is selected from the group consisting of a bond, —CH₂— and —CH₂CH₂—;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₂-C₁₄ alkylenyl or optionally substituted C₂-C₁₄ alkenylenyl;
Y¹ and Y² are independently selected from the group consisting of

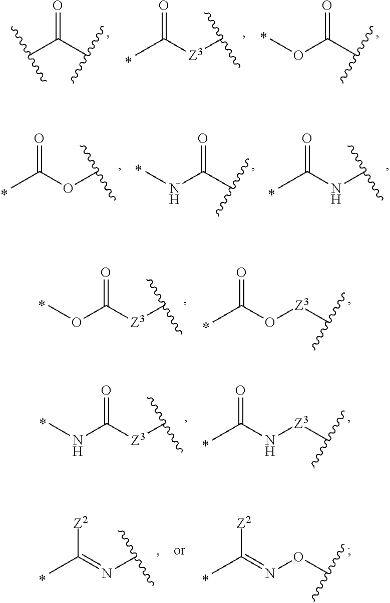

wherein the bond marked with an “*” is attached to X⁴ or X⁵;
each Z² is independently H or optionally substituted C₁-C₈ alkyl;
each Z³ is independently optionally substituted C₁-C₆ alkylenyl;
R² is selected from the group consisting of optionally substituted C₄-C₂₀ alkyl, optionally substituted C₂-C₁₄ alkenyl, and —(CH₂)ₚCH(OR⁶)(OR⁷);
R³ is selected from the group consisting of optionally substituted C₄-C₂₀ alkyl, optionally substituted C₂-C₁₄ alkenyl, or (CH₂)_{q}CH(OR⁸)(OR⁹);
R^{1a} is:

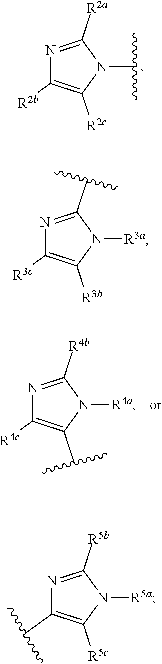

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
R⁶, R⁷, R⁸, and R⁹ are independently optionally substituted C₁-C₁₄ alkyl, optionally
substituted C₂-C₁₄ alkenyl, or —(CH₂)ₘ-A-(CH₂)ₙH;
each A is independently a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
p is selected from the group consisting of 0, 1, 2, 3, 4, 5, 6, and 7; and
q is selected from the group consisting of 0, 1, 2, 3, 4, 5, 6, and 7.

### Formulas (CY-I), (CY-II), (CY-III), (CY-IV), (CY-V), (CY-VI), (CY-VII), (CY-VIII), (CY-IX), (CY-IV-a), (CY-IV-b), (CY-IV-c), (CY-IV-d), (CY-IV-e), and (CY-IV-f)

In some embodiments, the present disclosure comprises a compound of any of the below Formulae:

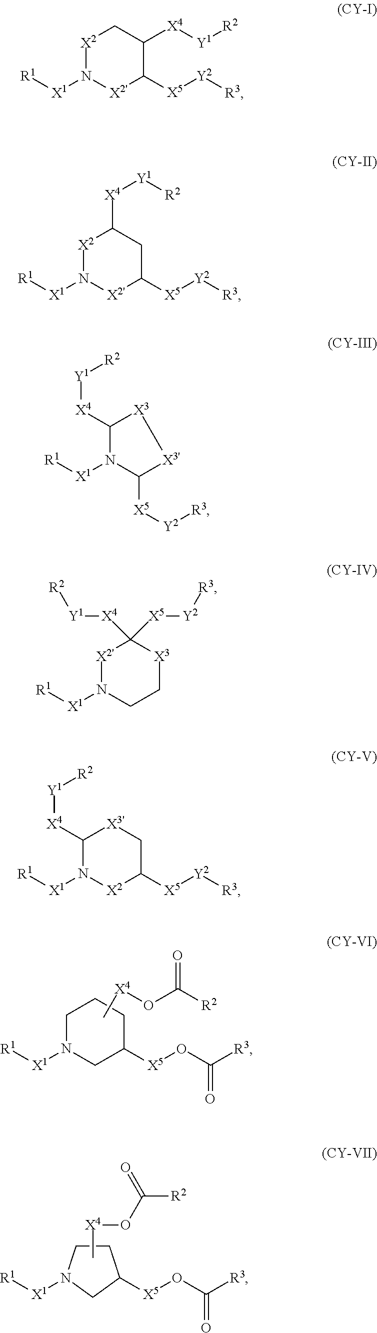

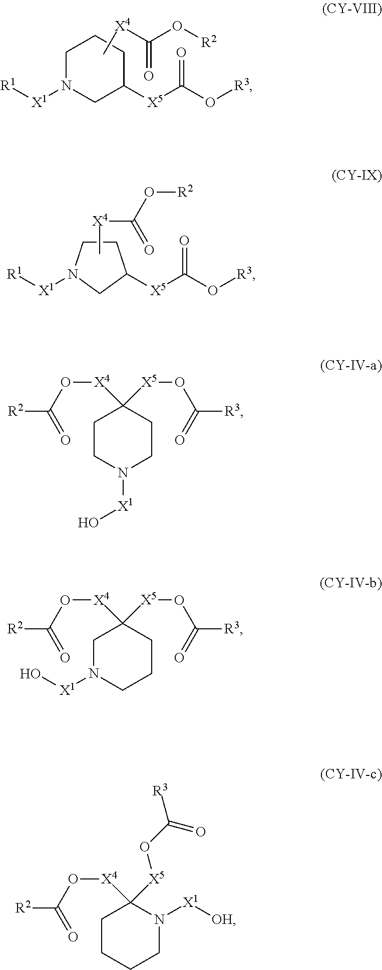

In some embodiments, the present disclosure includes a compound of Formula (CY-IV-d), (CY-IV-e), or (CY-IV-f)

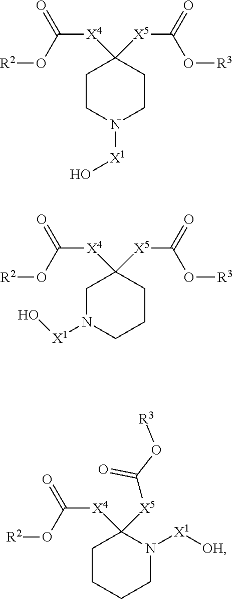

### Formula (CY-IV′)

In some embodiments, ionizable lipids of the present disclosure have a structure of Formula (CY-IV′):

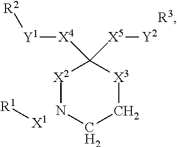

or a pharmaceutically acceptable salt thereof, wherein R¹, R², R³, X¹, X², X³, X⁴, X⁵, Y¹, and Y² are as defined in connection with Formula (CY-I′).

### Formula (CY-VI′)

In some embodiments, ionizable lipids of the present disclosure have a structure of Formula (CY-VI′):

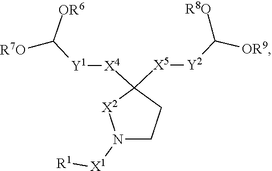

or a pharmaceutically acceptable salt thereof, wherein R¹, R⁶, R⁷, R⁸, R⁹, X¹, X², X³, X⁴, X⁵, Y¹, and Y² are as defined in connection with Formula (CY-I′).

In some embodiments, ionizable lipids of the present disclosure have a structure of Formula (CY-VI′), or a pharmaceutically acceptable salt thereof, wherein R1 is —OH.

In some embodiments, ionizable lipids of the present disclosure have a structure of Formula (CYVI′), or a pharmaceutically acceptable salt thereof, wherein X1 is C2-C6 alkylenyl.

In some embodiments, ionizable lipids of the present disclosure have a structure of Formula (CY-VI′), or a pharmaceutically acceptable salt thereof, wherein X2 is —CH2CH2-.

In some embodiments, ionizable lipids of the present disclosure have a structure of Formula (CYVI′), or a pharmaceutically acceptable salt thereof, wherein X4 is C2-C6 alkylenyl.

In some embodiments, ionizable lipids of the present disclosure have a structure of Formula (CYVI′), or a pharmaceutically acceptable salt thereof, wherein X5 is C2-C6 alkylenyl.

In some embodiments, ionizable lipids of the present disclosure have a structure of Formula (CY-VI′), or a pharmaceutically acceptable salt thereof, wherein Y¹ is:

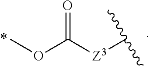

In some embodiments, ionizable lipids of the present disclosure have a structure of Formula (CY-VI′), or a pharmaceutically acceptable salt thereof, wherein Y² is:

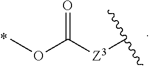

In some embodiments, ionizable lipids of the present disclosure have a structure of Formula (CYVI′), or a pharmaceutically acceptable salt thereof, wherein each Z3 is independently optionally substituted C1-C6 alkylenyl.

In some embodiments, ionizable lipids of the present disclosure have a structure of Formula (CY-VI′), or a pharmaceutically acceptable salt thereof, wherein each Z3 is —CH2CH2-.

In some embodiments, ionizable lipids of the present disclosure have a structure of Formula (CY-VI′), or a pharmaceutically acceptable salt thereof, wherein R6 is C5-C14 alkyl.

In some embodiments, ionizable lipids of the present disclosure have a structure of Formula (CY-VI′), or a pharmaceutically acceptable salt thereof, wherein R7 is C5-C14 alkyl.

In some embodiments, ionizable lipids of the present disclosure have a structure of Formula (CY-VI′), or a pharmaceutically acceptable salt thereof, wherein R6 is C6-C14 alkenyl.

In some embodiments, ionizable lipids of the present disclosure have a structure of Formula (CY-VI′), or a pharmaceutically acceptable salt thereof, wherein R7 is C6-C14 alkenyl.

In some embodiments, ionizable lipids of the present disclosure have a structure of Formula (CY-VI′), or a pharmaceutically acceptable salt thereof, wherein R⁸ is C₅-C₁₆ alkyl.

In some embodiments, ionizable lipids of the present disclosure have a structure of Formula (CY-VI′), or a pharmaceutically acceptable salt thereof, wherein R9 is C5-C14 alkyl.

In some embodiments, ionizable lipids of the present disclosure have a structure of Formula (CY-VI′), or a pharmaceutically acceptable salt thereof, wherein R8 is C6-C14 alkenyl.

In some embodiments, ionizable lipids of the present disclosure have a structure of Formula (CY-VI′), or a pharmaceutically acceptable salt thereof, wherein R9 is C6-C14 alkenyl.

In some embodiments, ionizable lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, Lipids of the Present Disclosure are selected from any lipid in Table (I-B) below or a pharmaceutically acceptable salt thereof:

TABLE (I-B)
Non-Limiting Examples of Ionizable Lipids with a Cyclic Core

| Structure | # |
|---|---|
| 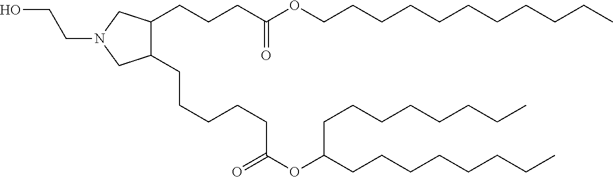 [SMILES: CCCCCCCCCCCOC(=O)CCCC1CN(CCO)CC1CCCCCC(=O)OC(CCCCCCCC)CCCCCCCC] | CY1 |
| 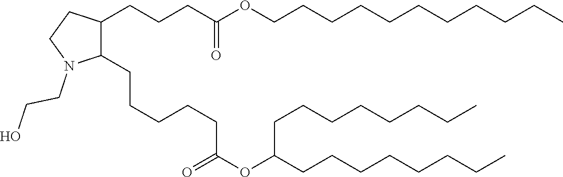 [SMILES: CCCCCCCCCCCOC(=O)CCCC1CCN(CCO)C1CCCCCC(=O)OC(CCCCCCCC)CCCCCCCC] | CY2 |
| 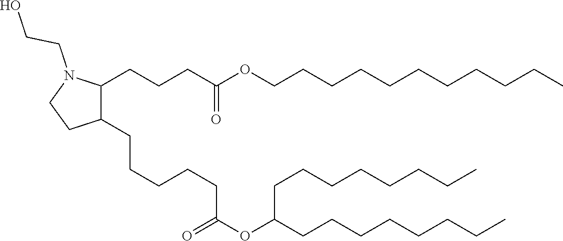 [SMILES: CCCCCCCCCCCOC(=O)CCCC1C(CCCCCC(=O)OC(CCCCCCCC)CCCCCCCC)CCN1CCO] | CY3 |
| 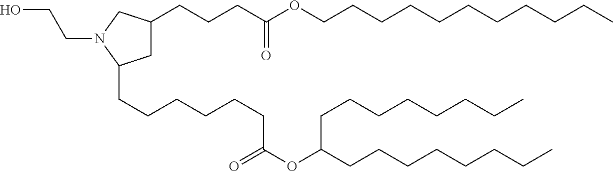 [SMILES: CCCCCCCCCCCOC(=O)CCCC1CC(CCCCCCC(=O)OC(CCCCCCCC)CCCCCCCC)N(CCO)C1] | CY4 |
| 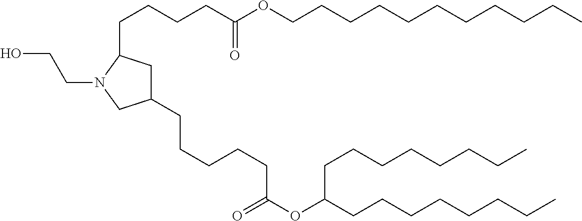 [SMILES: CCCCCCCCCCCOC(=O)CCCCC1CC(CCCCCC(=O)OC(CCCCCCCC)CCCCCCCC)CN1CCO] | CY5 |
| 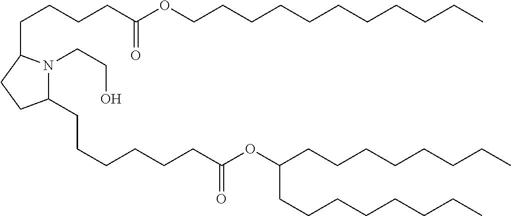 [SMILES: CCCCCCCCCCCOC(=O)CCCCC1CCC(CCCCCCC(=O)OC(CCCCCCCC)CCCCCCCC)N1CCO] | CY6 |
| 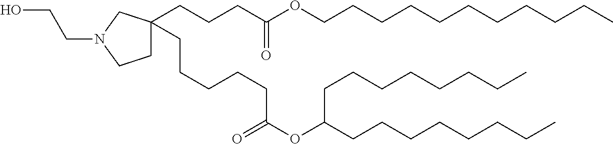 [SMILES: CCCCCCCCCCCOC(=O)CCCC1(CCCCCC(=O)OC(CCCCCCCC)CCCCCCCC)CCN(CCO)C1] | CY7 |
| 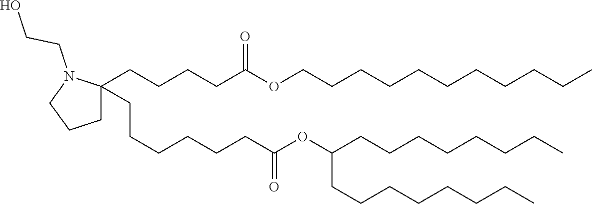 [SMILES: CCCCCCCCCCCOC(=O)CCCCC1(CCCCCCC(=O)OC(CCCCCCCC)CCCCCCCC)CCCN1CCO] | CY8 |
| 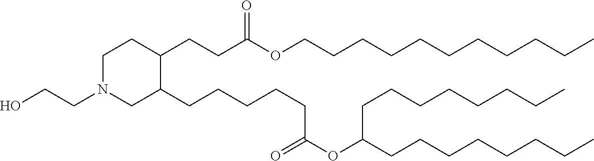 [SMILES: CCCCCCCCCCCOC(=O)CCC1CCN(CCO)CC1CCCCCC(=O)OC(CCCCCCCC)CCCCCCCC] | CY9 |
| 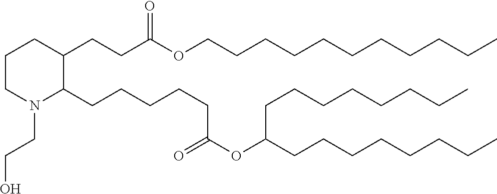 [SMILES: CCCCCCCCCCCOC(=O)CCC1CCCN(CCO)C1CCCCCC(=O)OC(CCCCCCCC)CCCCCCCC] | CY10 |
| 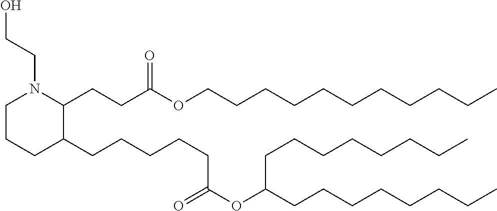 [SMILES: CCCCCCCCCCCOC(=O)CCC1C(CCCCCC(=O)OC(CCCCCCCC)CCCCCCCC)CCCN1CCO] | CY11 |
| 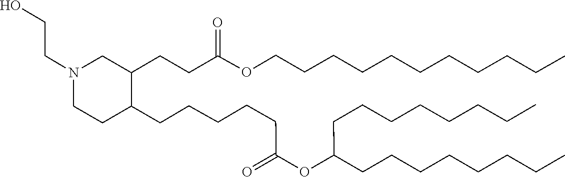 [SMILES: CCCCCCCCCCCOC(=O)CCC1CN(CCO)CCC1CCCCCC(=O)OC(CCCCCCCC)CCCCCCCC] | CY12 |
| 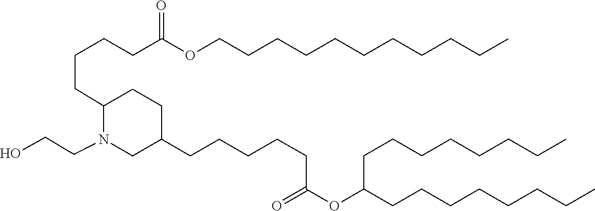 [SMILES: CCCCCCCCCCCOC(=O)CCCCC1CCC(CCCCCC(=O)OC(CCCCCCCC)CCCCCCCC)CN1CCO] | CY13 |
| 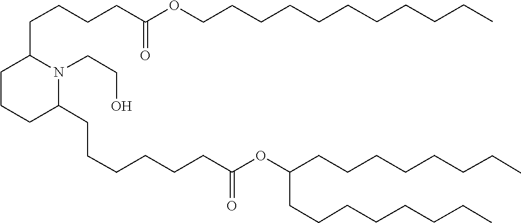 [SMILES: CCCCCCCCCCCOC(=O)CCCCC1CCCC(CCCCCCC(=O)OC(CCCCCCCC)CCCCCCCC)N1CCO] | CY14 |
| 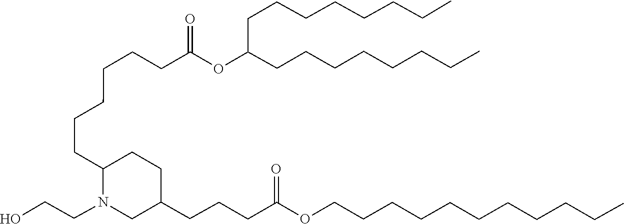 [SMILES: CCCCCCCCCCCOC(=O)CCCC1CCC(CCCCCCC(=O)OC(CCCCCCCC)CCCCCCCC)N(CCO)C1] | CY15 |
| 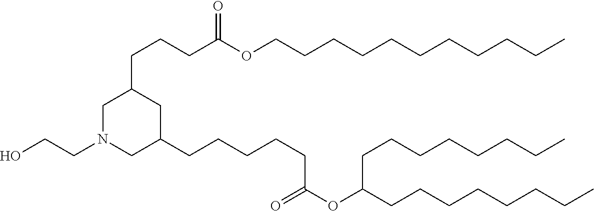 [SMILES: CCCCCCCCCCCOC(=O)CCCC1CC(CCCCCC(=O)OC(CCCCCCCC)CCCCCCCC)CN(CCO)C1] | CY16 |
| 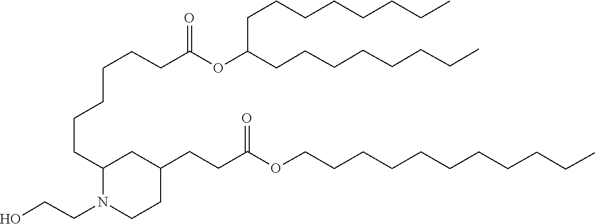 [SMILES: CCCCCCCCCCCOC(=O)CCC1CCN(CCO)C(CCCCCCC(=O)OC(CCCCCCCC)CCCCCCCC)C1] | CY17 |
| 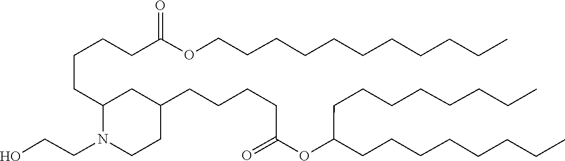 [SMILES: CCCCCCCCCCCOC(=O)CCCCC1CC(CCCCC(=O)OC(CCCCCCCC)CCCCCCCC)CCN1CCO] | CY18 |
| 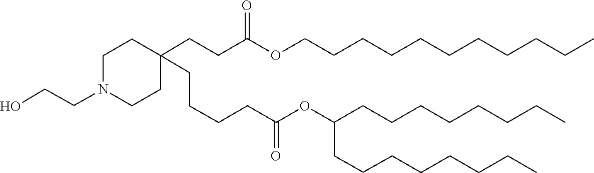 [SMILES: CCCCCCCCCCCOC(=O)CCC1(CCCCC(=O)OC(CCCCCCCC)CCCCCCCC)CCN(CCO)CC1] | CY19 |
| 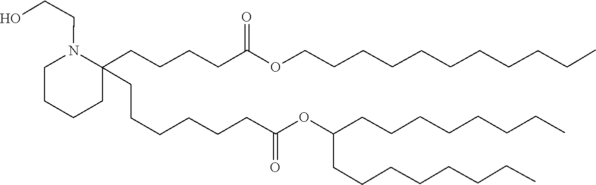 [SMILES: CCCCCCCCCCCOC(=O)CCCCC1(CCCCCCC(=O)OC(CCCCCCCC)CCCCCCCC)CCCCN1CCO] | CY20 |
| 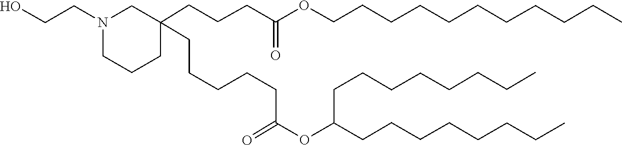 [SMILES: CCCCCCCCCCCOC(=O)CCCC1(CCCCCC(=O)OC(CCCCCCCC)CCCCCCCC)CCCN(CCO)C1] | CY21 |
| 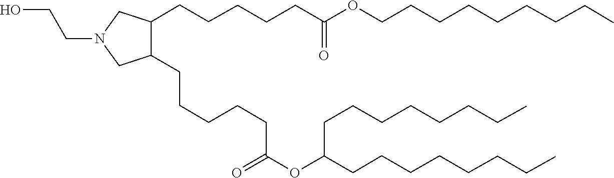 [SMILES: CCCCCCCCCOC(=O)CCCCCC1CN(CCO)CC1CCCCCC(=O)OC(CCCCCCCC)CCCCCCCC] | CY22 |
| 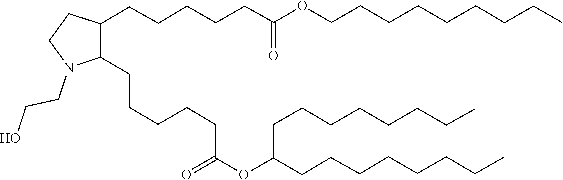 [SMILES: CCCCCCCCCOC(=O)CCCCCC1CCN(CCO)C1CCCCCC(=O)OC(CCCCCCCC)CCCCCCCC] | CY23 |
| 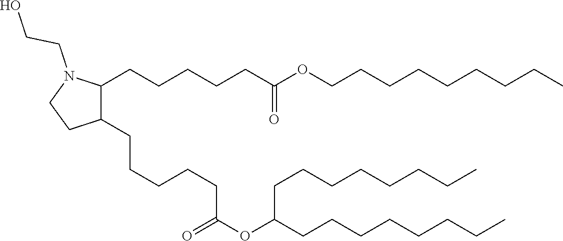 [SMILES: CCCCCCCCCOC(=O)CCCCCC1C(CCCCCC(=O)OC(CCCCCCCC)CCCCCCCC)CCN1CCO] | CY24 |
| 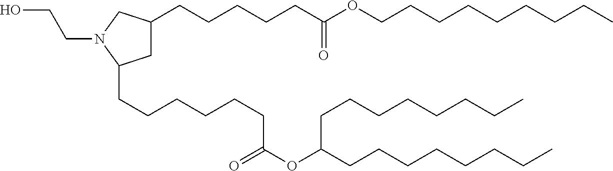 [SMILES: CCCCCCCCCOC(=O)CCCCCC1CC(CCCCCCC(=O)OC(CCCCCCCC)CCCCCCCC)N(CCO)C1] | CY25 |
| 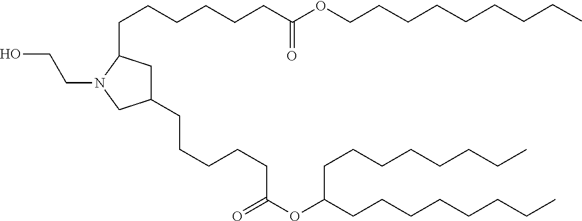 [SMILES: CCCCCCCCCOC(=O)CCCCCCC1CC(CCCCCC(=O)OC(CCCCCCCC)CCCCCCCC)CN1CCO] | CY26 |
| 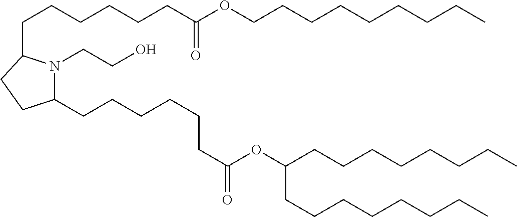 [SMILES: CCCCCCCCCOC(=O)CCCCCCC1CCC(CCCCCCC(=O)OC(CCCCCCCC)CCCCCCCC)N1CCO] | CY27 |
| 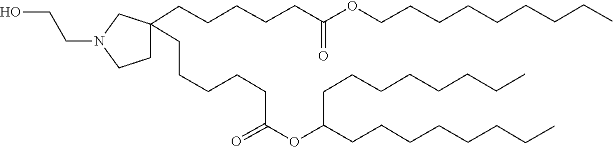 [SMILES: CCCCCCCCCOC(=O)CCCCCC1(CCCCCC(=O)OC(CCCCCCCC)CCCCCCCC)CCN(CCO)C1] | CY28 |
| 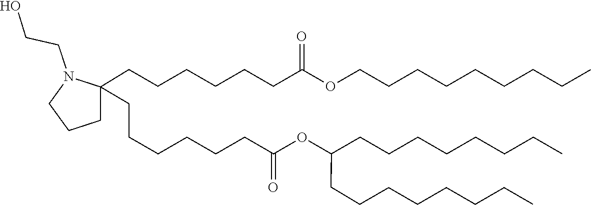 [SMILES: CCCCCCCCCOC(=O)CCCCCCC1(CCCCCCC(=O)OC(CCCCCCCC)CCCCCCCC)CCCN1CCO] | CY29 |
| 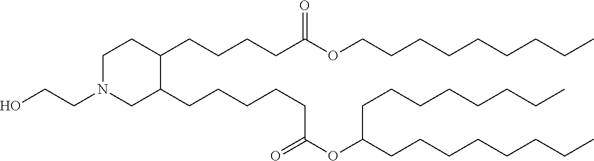 [SMILES: CCCCCCCCCOC(=O)CCCCC1CCN(CCO)CC1CCCCCC(=O)OC(CCCCCCCC)CCCCCCCC] | CY30 |
| 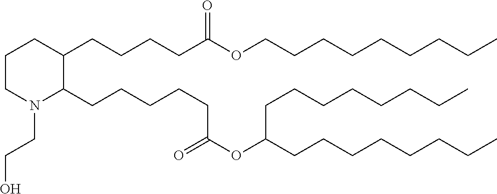 [SMILES: CCCCCCCCCOC(=O)CCCCC1CCCN(CCO)C1CCCCCC(=O)OC(CCCCCCCC)CCCCCCCC] | CY31 |
| 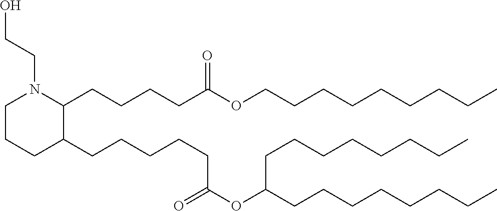 [SMILES: CCCCCCCCCOC(=O)CCCCC1C(CCCCCC(=O)OC(CCCCCCCC)CCCCCCCC)CCCN1CCO] | CY32 |
| 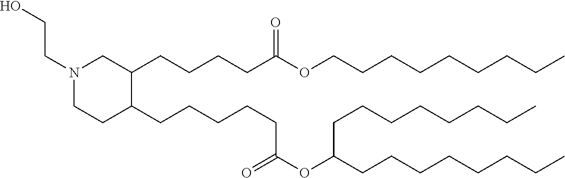 [SMILES: CCCCCCCCCOC(=O)CCCCC1CN(CCO)CCC1CCCCCC(=O)OC(CCCCCCCC)CCCCCCCC] | CY33 |
| 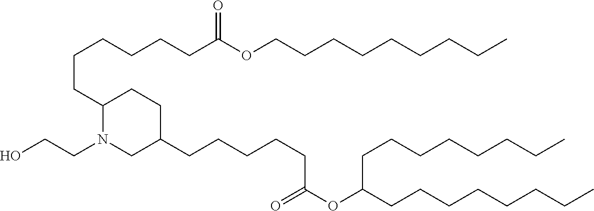 [SMILES: CCCCCCCCCOC(=O)CCCCCCC1CCC(CCCCCC(=O)OC(CCCCCCCC)CCCCCCCC)CN1CCO] | CY34 |
| 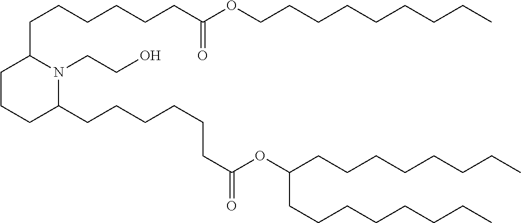 [SMILES: CCCCCCCCCOC(=O)CCCCCCC1CCCC(CCCCCCC(=O)OC(CCCCCCCC)CCCCCCCC)N1CCO] | CY35 |
| 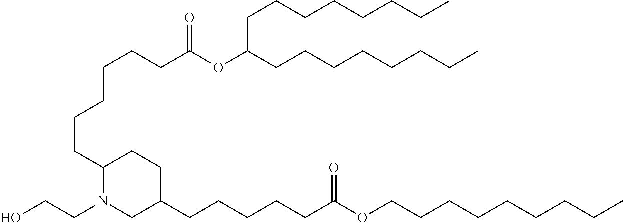 [SMILES: CCCCCCCCCOC(=O)CCCCCC1CCC(CCCCCCC(=O)OC(CCCCCCCC)CCCCCCCC)N(CCO)C1] | CY36 |
| 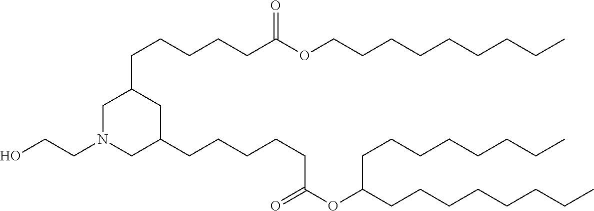 [SMILES: CCCCCCCCCOC(=O)CCCCCC1CC(CCCCCC(=O)OC(CCCCCCCC)CCCCCCCC)CN(CCO)C1] | CY37 |
| 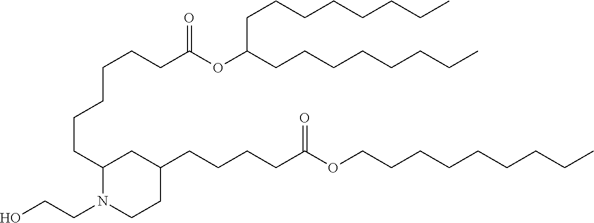 [SMILES: CCCCCCCCCOC(=O)CCCCC1CCN(CCO)C(CCCCCCC(=O)OC(CCCCCCCC)CCCCCCCC)C1] | CY38 |
| 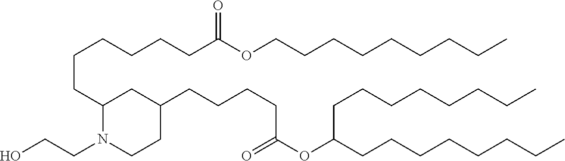 [SMILES: CCCCCCCCCOC(=O)CCCCCCC1CC(CCCCC(=O)OC(CCCCCCCC)CCCCCCCC)CCN1CCO] | CY39 |
| 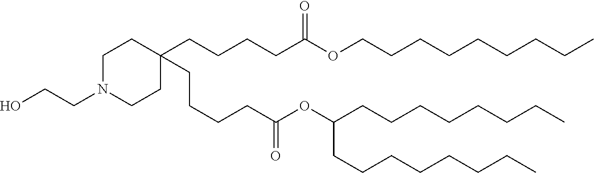 [SMILES: CCCCCCCCCOC(=O)CCCCC1(CCCCC(=O)OC(CCCCCCCC)CCCCCCCC)CCN(CCO)CC1] | CY40 |
| 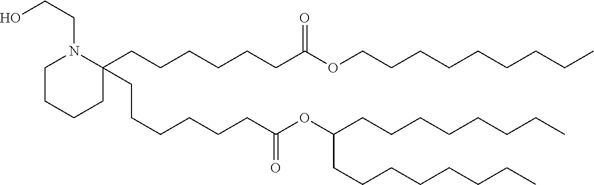 [SMILES: CCCCCCCCCOC(=O)CCCCCCC1(CCCCCCC(=O)OC(CCCCCCCC)CCCCCCCC)CCCCN1CCO] | CY41 |
| 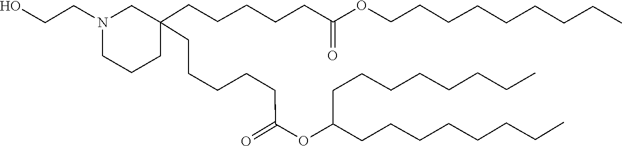 [SMILES: CCCCCCCCCOC(=O)CCCCCC1(CCCCCC(=O)OC(CCCCCCCC)CCCCCCCC)CCCN(CCO)C1] | CY42 |
| 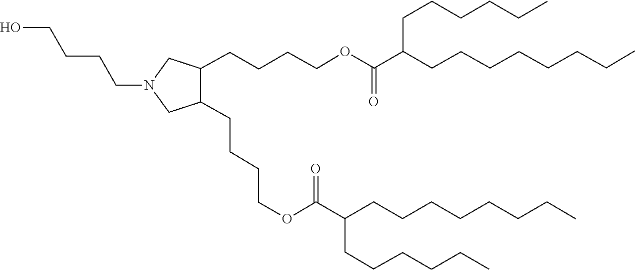 [SMILES: CCCCCCCCC(CCCCCC)C(=O)OCCCCC1CN(CCCCO)CC1CCCCOC(=O)C(CCCCCC)CCCCCCCC] | CY43 |
| 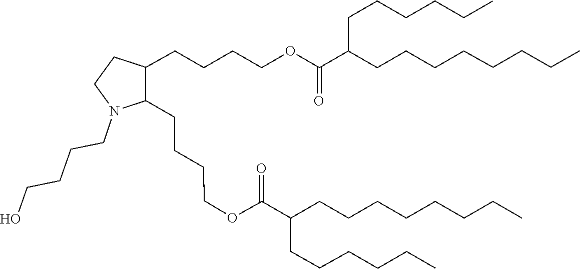 [SMILES: CCCCCCCCC(CCCCCC)C(=O)OCCCCC1CCN(CCCCO)C1CCCCOC(=O)C(CCCCCC)CCCCCCCC] | CY44 |
| 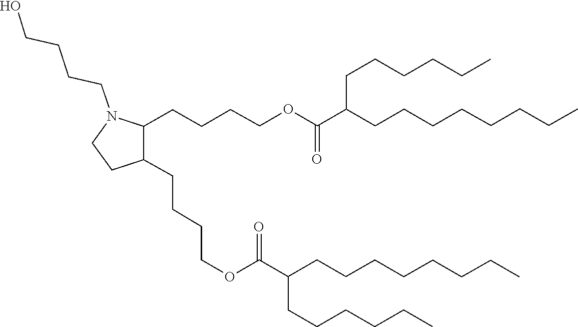 [SMILES: CCCCCCCCC(CCCCCC)C(=O)OCCCCC1CCN(CCCCO)C1CCCCOC(=O)C(CCCCCC)CCCCCCCC] | CY45 |
| 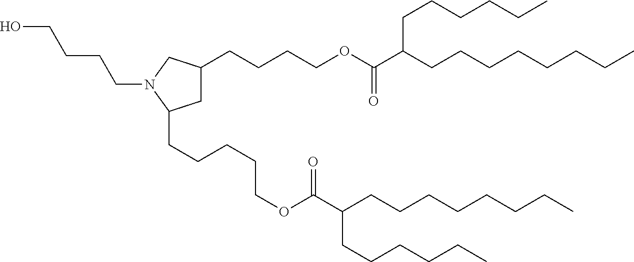 [SMILES: CCCCCCCCC(CCCCCC)C(=O)OCCCCCC1CC(CCCCOC(=O)C(CCCCCC)CCCCCCCC)CN1CCCCO] | CY46 |
| 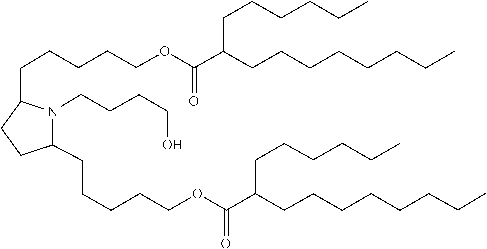 [SMILES: CCCCCCCCC(CCCCCC)C(=O)OCCCCCC1CCC(CCCCCOC(=O)C(CCCCCC)CCCCCCCC)N1CCCCO] | CY47 |
| 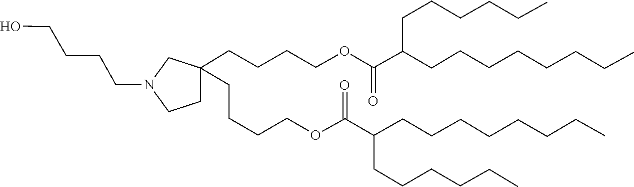 [SMILES: CCCCCCCCC(CCCCCC)C(=O)OCCCCC1(CCCCOC(=O)C(CCCCCC)CCCCCCCC)CCN(CCCCO)C1] | CY48 |
| 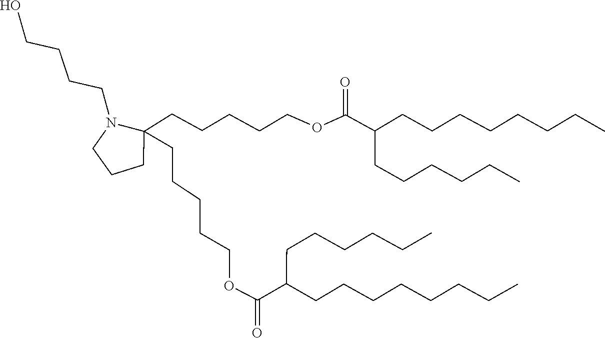 [SMILES: CCCCCCCCC(CCCCCC)C(=O)OCCCCCC1(CCCCCOC(=O)C(CCCCCC)CCCCCCCC)CCCN1CCCCO] | CY49 |
| 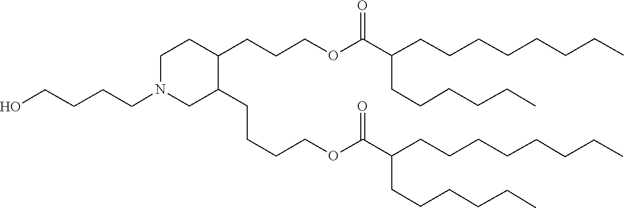 [SMILES: CCCCCCCCC(CCCCCC)C(=O)OCCCCC1CN(CCCCO)CCC1CCCOC(=O)C(CCCCCC)CCCCCCCC] | CY50 |
| 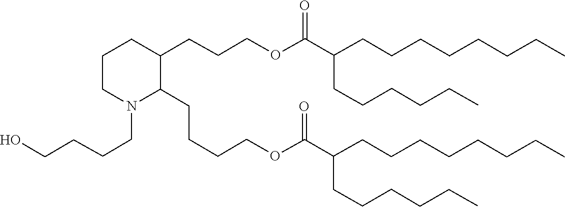 [SMILES: CCCCCCCCC(CCCCCC)C(=O)OCCCCC1C(CCCOC(=O)C(CCCCCC)CCCCCCCC)CCCN1CCCCO] | CY51 |
| 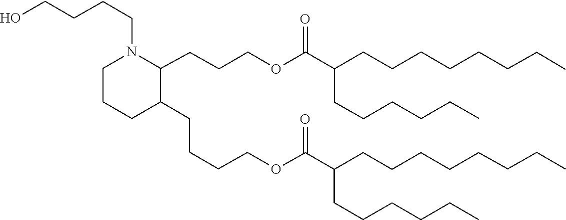 [SMILES: CCCCCCCCC(CCCCCC)C(=O)OCCCCC1CCCN(CCCCO)C1CCCOC(=O)C(CCCCCC)CCCCCCCC] | CY52 |
| 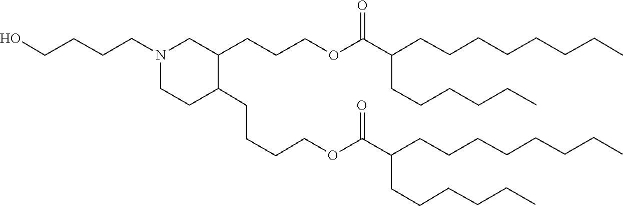 [SMILES: CCCCCCCCC(CCCCCC)C(=O)OCCCCC1CCN(CCCCO)CC1CCCOC(=O)C(CCCCCC)CCCCCCCC] | CY53 |
| 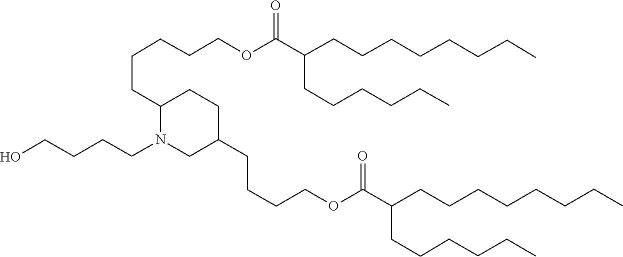 [SMILES: CCCCCCCCC(CCCCCC)C(=O)OCCCCCC1CCC(CCCCOC(=O)C(CCCCCC)CCCCCCCC)CN1CCCCO] | CY54 |
| 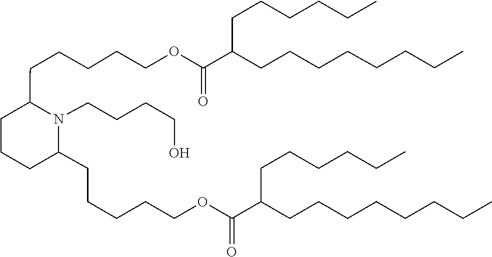 [SMILES: CCCCCCCCC(CCCCCC)C(=O)OCCCCCC1CCCC(CCCCCOC(=O)C(CCCCCC)CCCCCCCC)N1CCCCO] | CY55 |
| 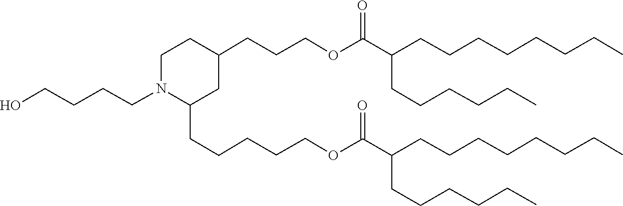 [SMILES: CCCCCCCCC(CCCCCC)C(=O)OCCCCCC1CC(CCCOC(=O)C(CCCCCC)CCCCCCCC)CCN1CCCCO] | CY56 |
| 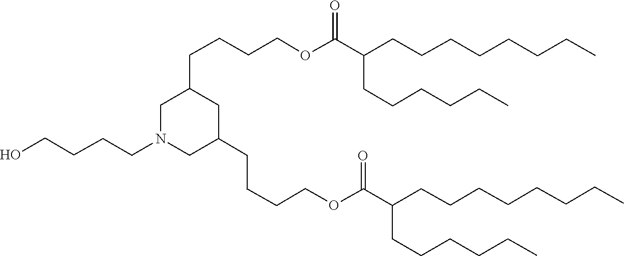 [SMILES: CCCCCCCCC(CCCCCC)C(=O)OCCCCC1CC(CCCCOC(=O)C(CCCCCC)CCCCCCCC)CN(CCCCO)C1] | CY57 |
| 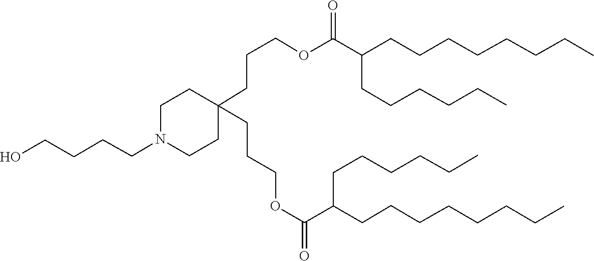 [SMILES: CCCCCCCCC(CCCCCC)C(=O)OCCCC1(CCCOC(=O)C(CCCCCC)CCCCCCCC)CCN(CCCCO)CC1] | CY58 |
| 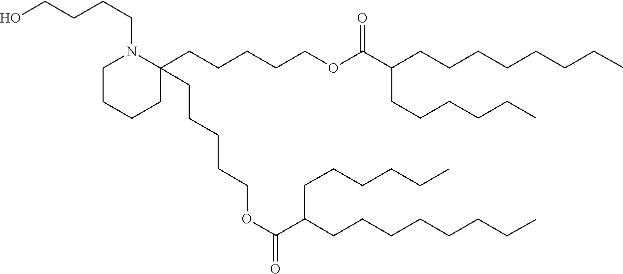 [SMILES: CCCCCCCCC(CCCCCC)C(=O)OCCCCCC1(CCCCCOC(=O)C(CCCCCC)CCCCCCCC)CCCCN1CCCCO] | CY59 |
| 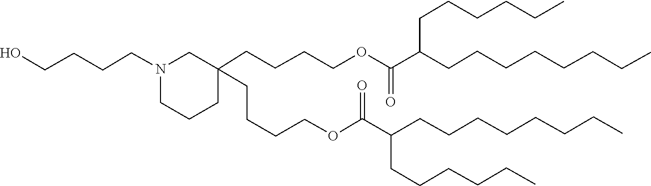 [SMILES: CCCCCCCCC(CCCCCC)C(=O)OCCCCC1(CCCCOC(=O)C(CCCCCC)CCCCCCCC)CCCN(CCCCO)C1] | CY60 |
| 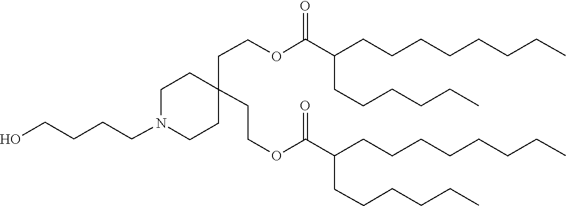 [SMILES: CCCCCCCCC(CCCCCC)C(=O)OCCC1(CCOC(=O)C(CCCCCC)CCCCCCCC)CCN(CCCCO)CC1] | CY61 |
| 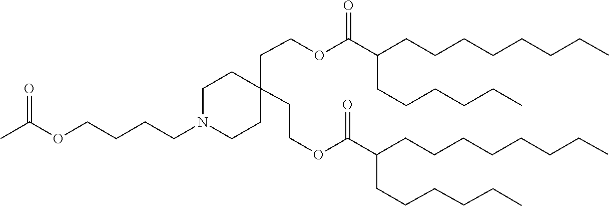 [SMILES: CCCCCCCCC(CCCCCC)C(=O)OCCC1(CCOC(=O)C(CCCCCC)CCCCCCCC)CCN(CCCCOC(C)=O)CC1] | CY62 |
| 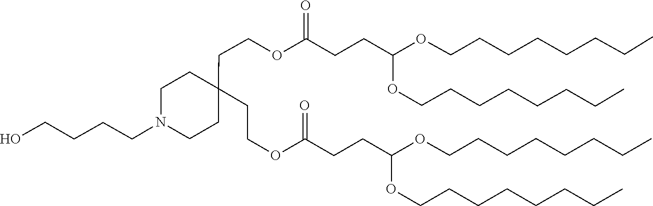 [SMILES: CCCCCCCCOC(CCC(=O)OCCC1(CCOC(=O)CCC(OCCCCCCCC)OCCCCCCCC)CCN(CCCCO)CC1)OCCCCCCCC] | CY63 |
| 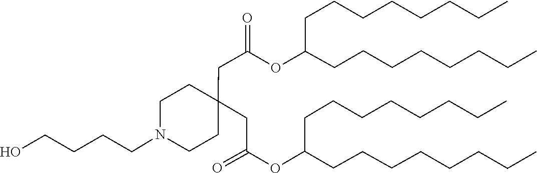 [SMILES: CCCCCCCCC(CCCCCCCC)OC(=O)CC1(CC(=O)OC(CCCCCCCC)CCCCCCCC)CCN(CCCCO)CC1] | CY64 |
| 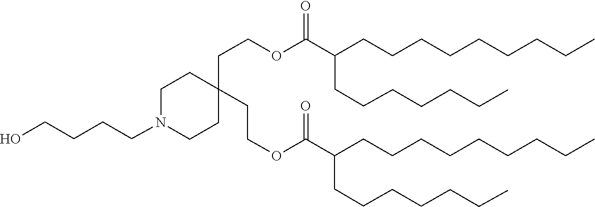 [SMILES: CCCCCCCCCC(CCCCCCC)C(=O)OCCC1(CCOC(=O)C(CCCCCCC)CCCCCCCCC)CCN(CCCCO)CC1] | CY65 |
| 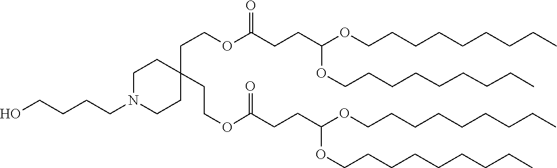 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC] | CY66 |
| 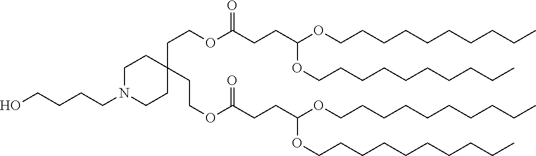 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC] | CY67 |
| 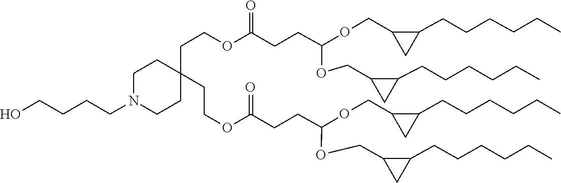 [SMILES: CCCCCCC1CC1COC(CCC(=O)OCCC1(CCOC(=O)CCC(OCC2CC2CCCCCC)OCC2CC2CCCCCC)CCN(CCCCO)CC1)OCC1CC1CCCCCC] | CY68 |
| 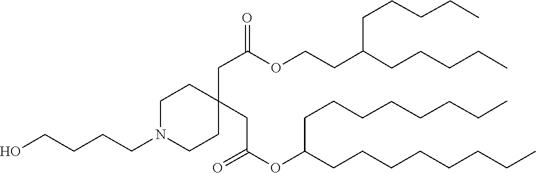 [SMILES: CCCCCCCCC(CCCCCCCC)OC(=O)CC1(CC(=O)OCCC(CCCCC)CCCCC)CCN(CCCCO)CC1] | CY69 |
| 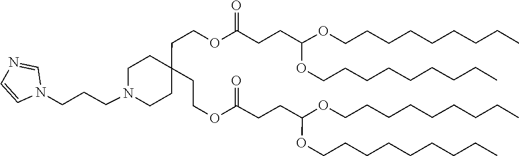 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC] | CY70 |
| 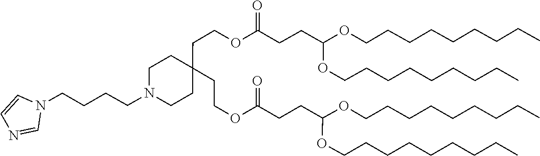 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC] | CY71 |

### Series “C”

In some embodiments, an LNP of the present disclosure comprises an ionizable lipid disclosed in PCT Publication WO2023122752A1, which is incorporated by reference herein, in its entirety.

In one embodiment, the disclosure provides a compound of Formula IA:

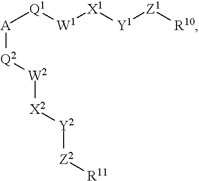

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

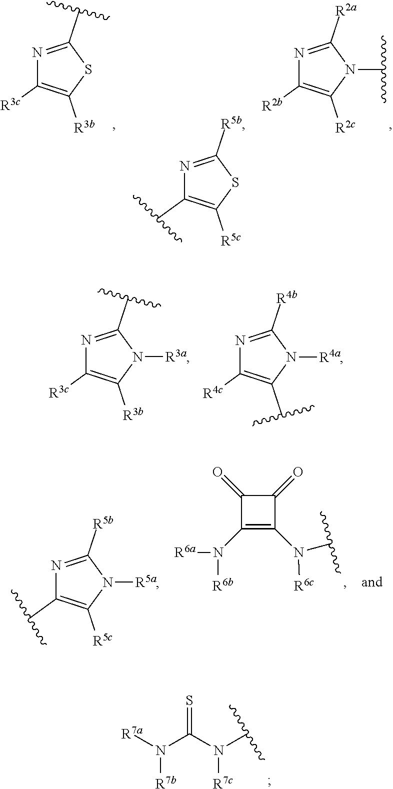

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

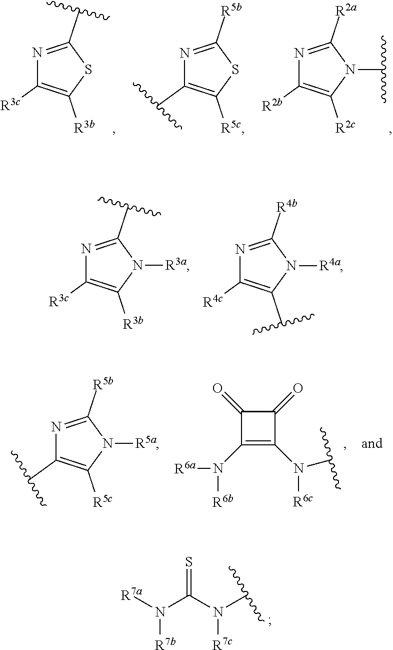

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

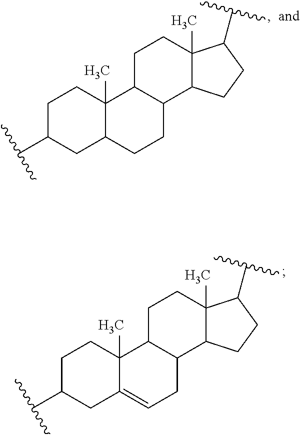 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b}) and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

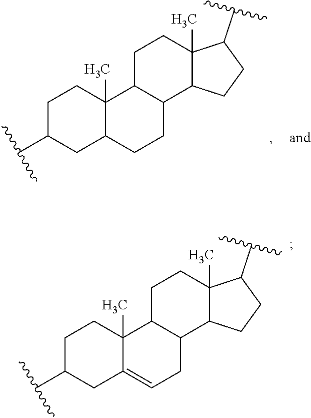 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl; wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IB:

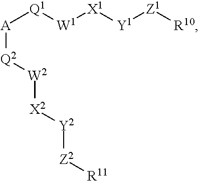

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

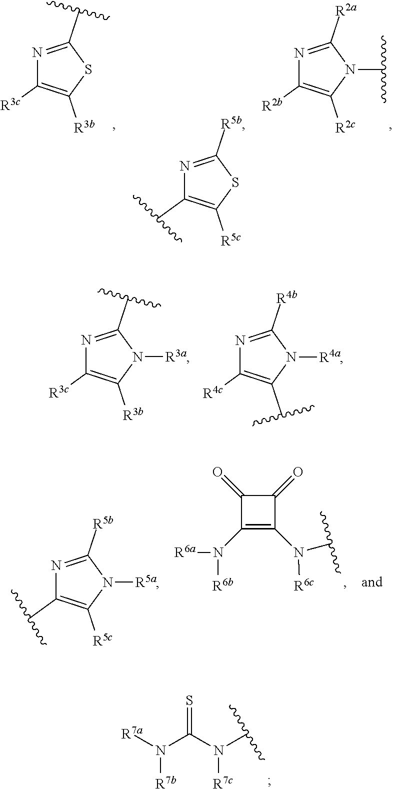

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

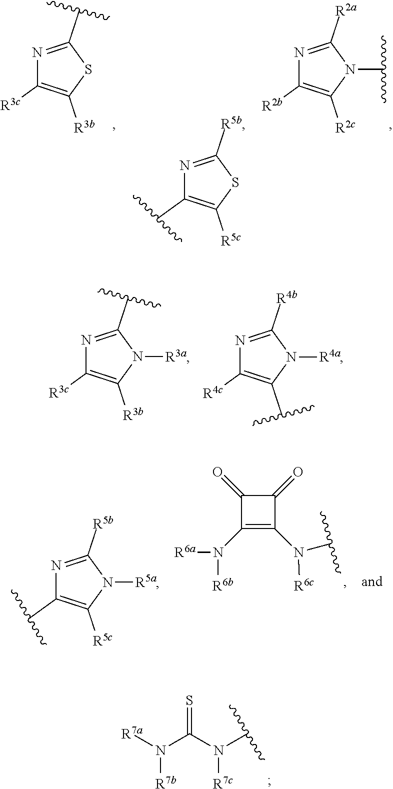

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{1a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₅-C₁₂ bridged cycloalkylenyl,

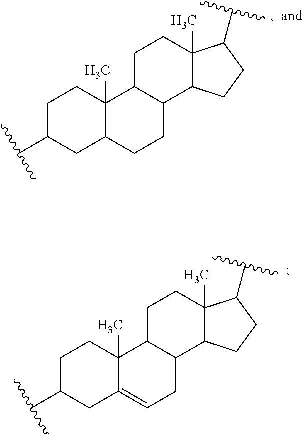 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

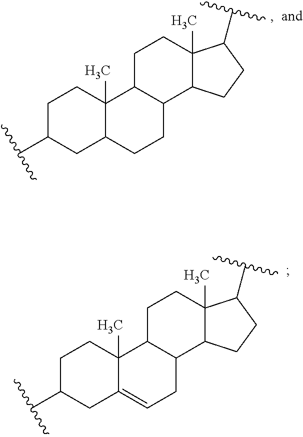 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl; wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IC:

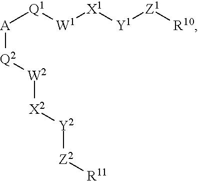

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

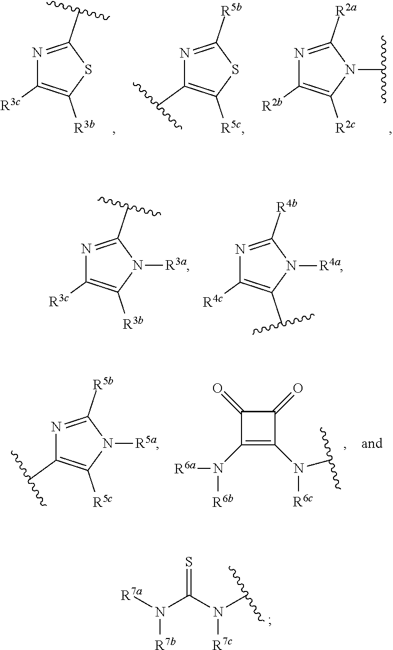

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R⁷, is selected from the group consisting of hydrogen and C₁C₆ alkyl;
R¹ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

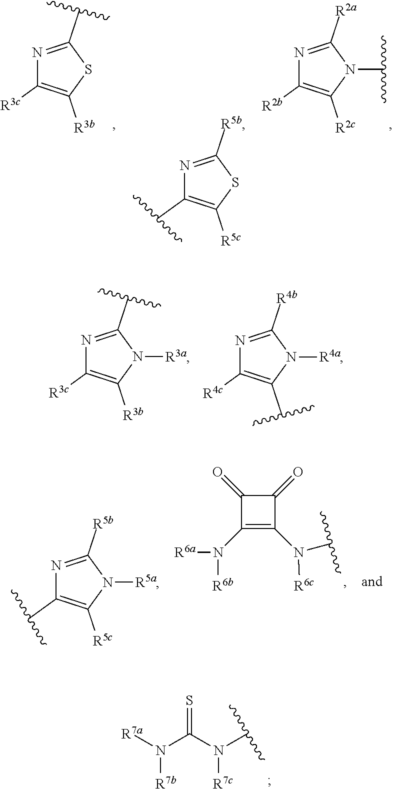

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

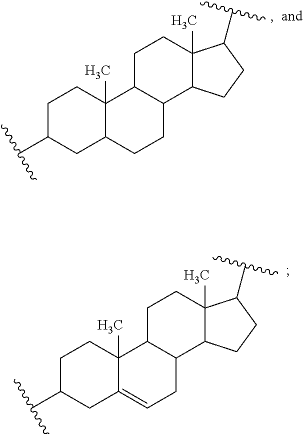 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is selected from the group consisting of —(CH₂)ₙ—, —O—, —S—, and —S—S—;
n is 0, 1, 2, 3, 4, 5, or 6;
Z² is of —(CH₂)ₚ—;
p is 0 or 1; and
R¹¹ is C₁-C₂₀ branched alkyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In some embodiments, the disclosure provides a compound of any one of Formulae IA, IB, IC, or a pharmaceutically acceptable salt or solvate thereof, wherein Z¹ is optionally substituted C₅-C₁₂ bridged cycloalkylenyl.

In some embodiments, the disclosure provides a compound of any one of Formulae IA, IB, IC, or a pharmaceutically acceptable salt or solvate thereof, wherein Z1 is not adamantyl.

In one embodiment, the disclosure provides a compound of Formula ID:

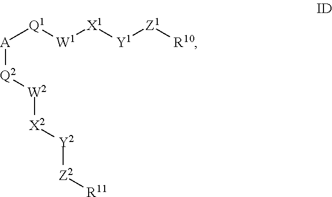

or a pharmaceutically acceptable salt or solvate thereof, wherein:
A is selected from the group consisting of —N(R^{1a})— and —C(R¹)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

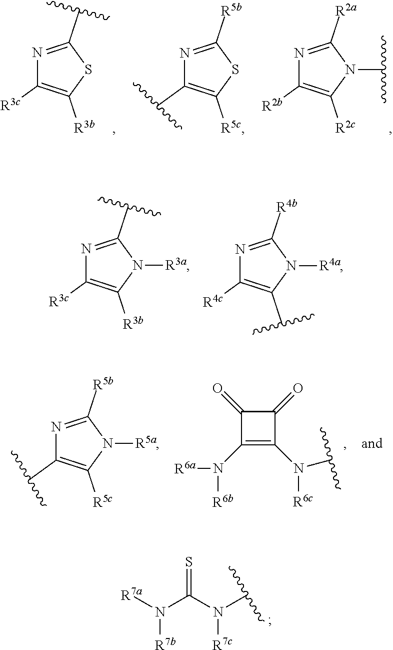

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

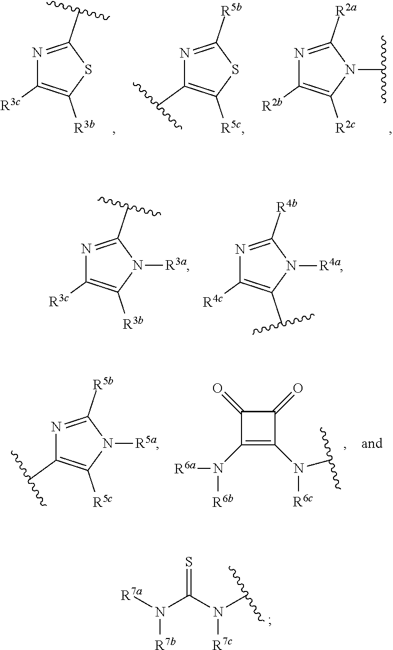

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is optionally substituted C₅-C₁₂ bridged cycloalkylenyl;
R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is —(CH₂)ₙ—;
n is 0, 1, 2, 3, 4, 5, or 6;
Z² is of —(CH₂)ₚ—;
p is 0 or 1; and
R¹¹ is C₁-C₂₀ branched alkyl.

In some embodiments, the disclosure provides a compound of Formula ID or a pharmaceutically acceptable salt or solvate thereof, wherein Z1 is not adamantyl.

In one embodiment, the disclosure provides a compound of Formula I:

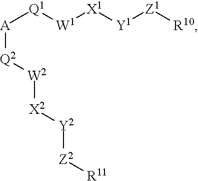

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl;
R¹ is selected from the group consisting of —OH,

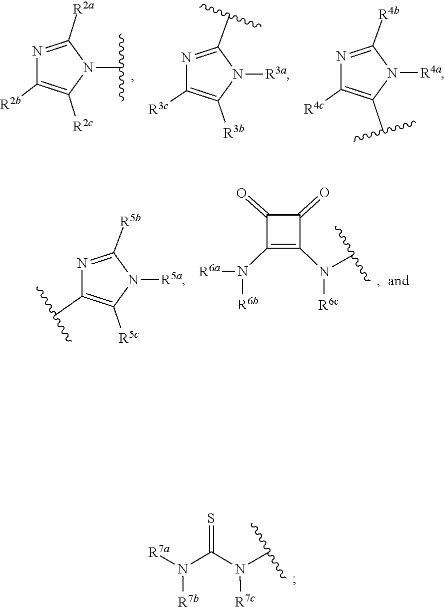

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R⁷, is selected from the group consisting of hydrogen and C₁C₆ alkyl;
R¹ is selected from the group consisting of hydrogen and C₁-C₆ alkyl;
R^{8a} is -L²-R⁸;
L² is C₂-C₆ alkylenyl;
R⁸ is —NR^{9a}R^{9b};
R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of C₄-C₁₂ cycloalkylenyl,

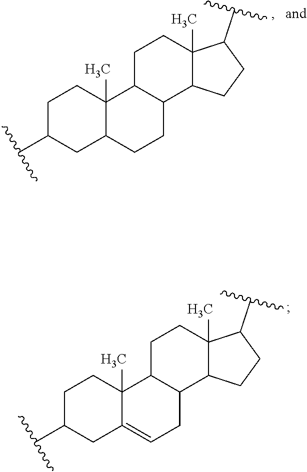 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})— and —OC(═O)O—;
R^{12b} is selected from the group consisting of hydrogen and C₁-C₆ alkyl;
X² is C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, C₄-C₁₂ cycloalkylenyl,

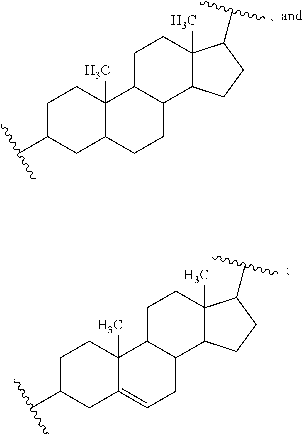 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or1; and
R¹¹ is selected from the group consisting of hydrogen, C₁-C₁₀ alkyl, and C₂-C₁₀ alkenyl.

In another embodiment, the disclosure provides a compound of Formula II, III, VI, VI′, VI″, VI″′, VII, VII′, VII″, VII″′, VIII, VIII′, VIII″, VIII″′, IX, IX′, IX″, IX″′, X, XIV′, XIV″, XIV″′, XV, XV′, XV″, XV″′, XVI, XVI′, XVI″, XVI″′, XVII, XVIII, XVIII′, XIX, XX, or XXI, as described in PCT Publication WO2023122752A1:

TABLE I-C
Non-Limiting Examples of Ionizable Lipids with a Constrained Arm

| # | Structure |
|---|---|
| C1 | 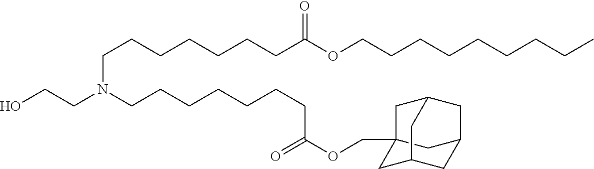 [SMILES: CCCCCCCCCOC(=O)CCCCCCCN(CCO)CCCCCCCC(=O)OCC12CC3CC(CC(C3)C1)C2] |
| C2 | 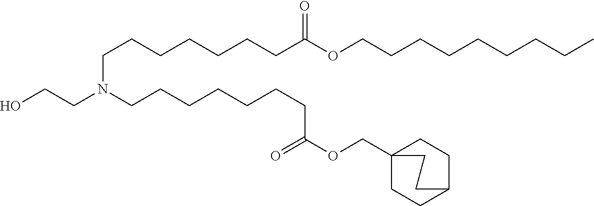 [SMILES: CCCCCCCCCOC(=O)CCCCCCCN(CCO)CCCCCCCC(=O)OCC12CCC(CC1)CC2] |
| C3 | 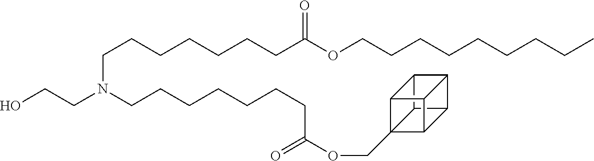 [SMILES: CCCCCCCCCOC(=O)CCCCCCCN(CCO)CCCCCCCC(=O)OCC12C3C4C5C3C1C5C42] |
| C4 | 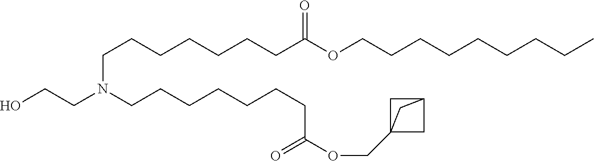 [SMILES: CCCCCCCCCOC(=O)CCCCCCCN(CCO)CCCCCCCC(=O)OCC12CC(C1)C2] |
| C5 | 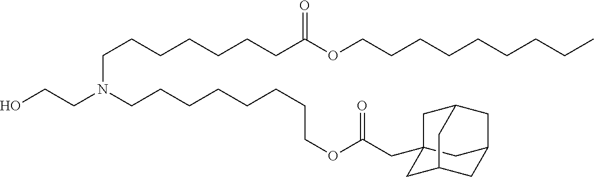 [SMILES: CCCCCCCCCOC(=O)CCCCCCCN(CCO)CCCCCCCCOC(=O)CC12CC3CC(CC(C3)C1)C2] |
| C6 | 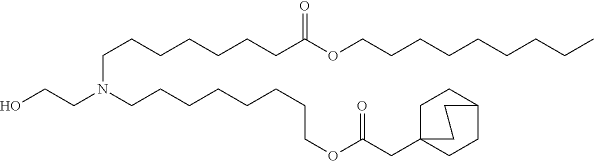 [SMILES: CCCCCCCCCOC(=O)CCCCCCCN(CCO)CCCCCCCCOC(=O)CC12CCC(CC1)CC2] |
| C7 | 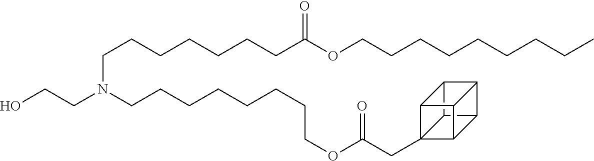 [SMILES: CCCCCCCCCOC(=O)CCCCCCCN(CCO)CCCCCCCCOC(=O)CC12C3C4C5C3C1C5C42] |
| C8 | 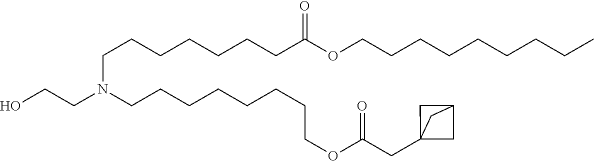 [SMILES: CCCCCCCCCOC(=O)CCCCCCCN(CCO)CCCCCCCCOC(=O)CC12CC(C1)C2] |
| C9 | 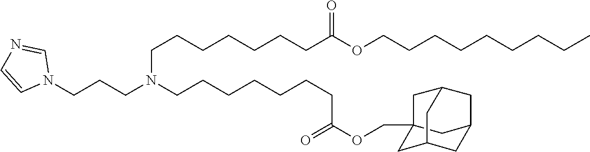 [SMILES: CCCCCCCCCOC(=O)CCCCCCCN(CCCCCCCC(=O)OCC12CC3CC(CC(C3)C1)C2)CCCn1ccnc1] |
| C10 | 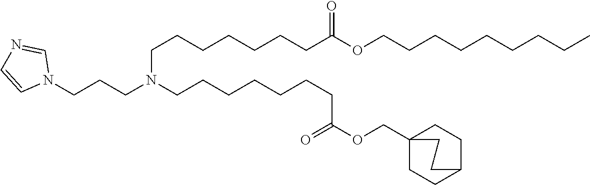 [SMILES: CCCCCCCCCOC(=O)CCCCCCCN(CCCCCCCC(=O)OCC12CCC(CC1)CC2)CCCn1ccnc1] |
| C11 | 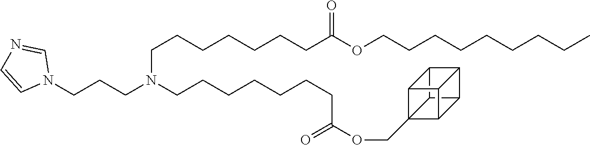 [SMILES: CCCCCCCCCOC(=O)CCCCCCCN(CCCCCCCC(=O)OCC12C3C4C5C3C1C5C42)CCCn1ccnc1] |
| C12 | 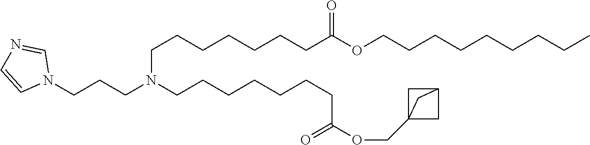 [SMILES: CCCCCCCCCOC(=O)CCCCCCCN(CCCCCCCC(=O)OCC12CC(C1)C2)CCCn1ccnc1] |
| C13 | 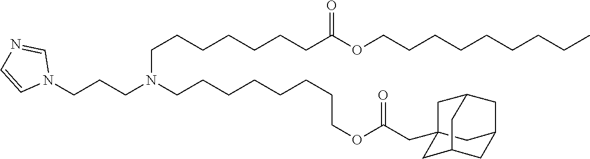 [SMILES: CCCCCCCCCOC(=O)CCCCCCCN(CCCCCCCCOC(=O)CC12CC3CC(CC(C3)C1)C2)CCCn1ccnc1] |
| C14 | 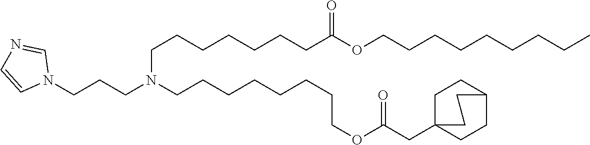 [SMILES: CCCCCCCCCOC(=O)CCCCCCCN(CCCCCCCCOC(=O)CC12CCC(CC1)CC2)CCCn1ccnc1] |
| C15 | 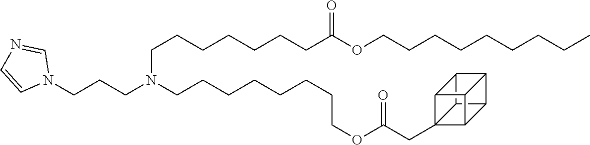 [SMILES: CCCCCCCCCOC(=O)CCCCCCCN(CCCCCCCCOC(=O)CC12C3C4C5C3C1C5C42)CCCn1ccnc1] |
| C16 | 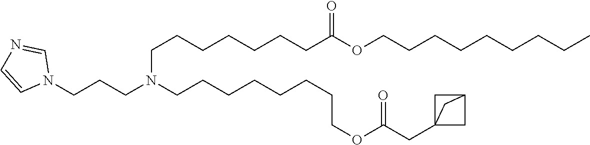 [SMILES: CCCCCCCCCOC(=O)CCCCCCCN(CCCCCCCCOC(=O)CC12CC(C1)C2)CCCn1ccnc1] |
| C17 | 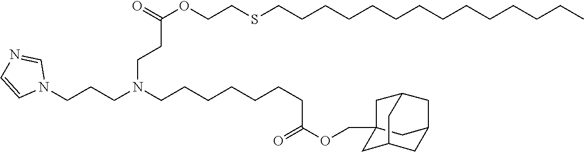 [SMILES: CCCCCCCCCCCCCCSCCOC(=O)CCN(CCCCCCCC(=O)OCC12CC3CC(CC(C3)C1)C2)CCCn1ccnc1] |
| C18 | 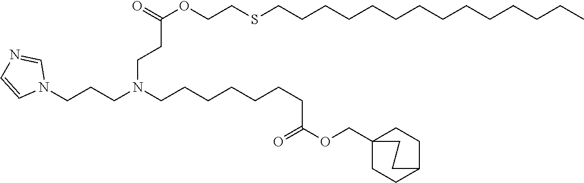 [SMILES: CCCCCCCCCCCCCCSCCOC(=O)CCN(CCCCCCCC(=O)OCC12CCC(CC1)CC2)CCCn1ccnc1] |
| C19 | 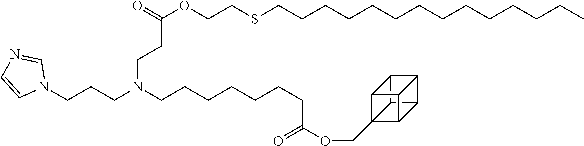 [SMILES: CCCCCCCCCCCCCCSCCOC(=O)CCN(CCCCCCCC(=O)OCC12C3C4C5C3C1C5C42)CCCn1ccnc1] |
| C20 | 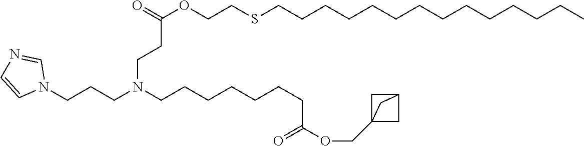 [SMILES: CCCCCCCCCCCCCCSCCOC(=O)CCN(CCCCCCCC(=O)OCC12CC(C1)C2)CCCn1ccnc1] |
| C21 | 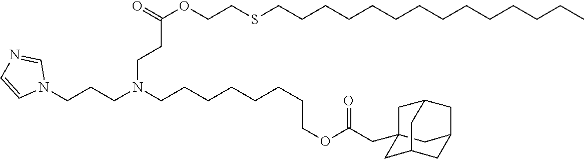 [SMILES: CCCCCCCCCCCCCCSCCOC(=O)CCN(CCCCCCCCOC(=O)CC12CC3CC(CC(C3)C1)C2)CCCn1ccnc1] |
| C22 | 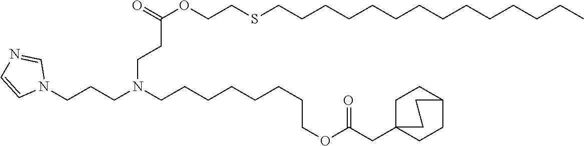 [SMILES: CCCCCCCCCCCCCCSCCOC(=O)CCN(CCCCCCCCOC(=O)CC12CCC(CC1)CC2)CCCn1ccnc1] |
| C23 | 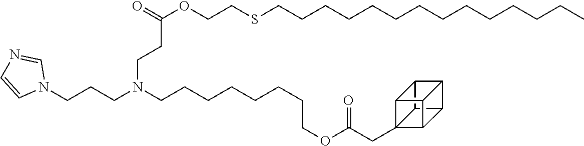 [SMILES: CCCCCCCCCCCCCCSCCOC(=O)CCN(CCCCCCCCOC(=O)CC12C3C4C5C3C1C5C42)CCCn1ccnc1] |
| C24 | 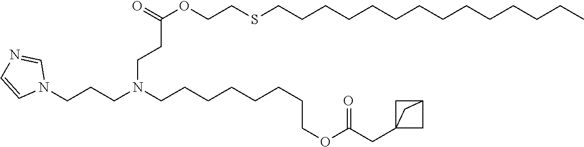 [SMILES: CCCCCCCCCCCCCCSCCOC(=O)CCN(CCCCCCCCOC(=O)CC12CC(C1)C2)CCCn1ccnc1] |
| C25 | 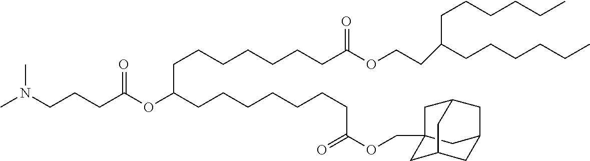 [SMILES: CCCCCCC(CCCCCC)CCOC(=O)CCCCCCCC(CCCCCCCC(=O)OCC12CC3CC(CC(C3)C1)C2)OC(=O)CCCN(C)C] |
| C26 | 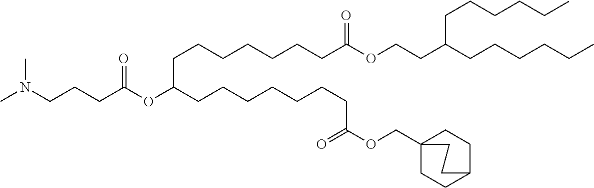 [SMILES: CCCCCCC(CCCCCC)CCOC(=O)CCCCCCCC(CCCCCCCC(=O)OCC12CCC(CC1)CC2)OC(=O)CCCN(C)C] |
| C27 | 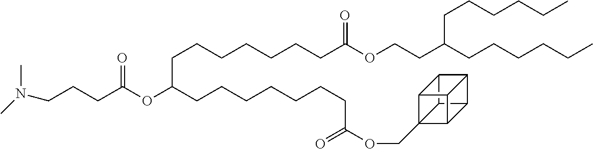 [SMILES: CCCCCCC(CCCCCC)CCOC(=O)CCCCCCCC(CCCCCCCC(=O)OCC12C3C4C5C3C1C5C42)OC(=O)CCCN(C)C] |
| C28 | 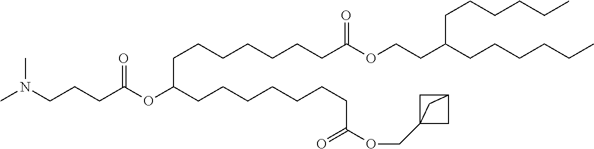 [SMILES: CCCCCCC(CCCCCC)CCOC(=O)CCCCCCCC(CCCCCCCC(=O)OCC12CC(C1)C2)OC(=O)CCCN(C)C] |
| C29 | 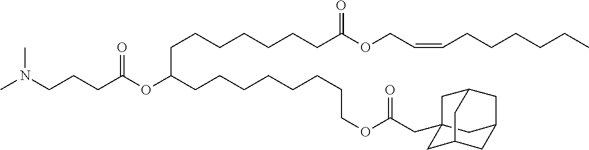 [SMILES: CCCCCC/C=C\COC(=O)CCCCCCCC(CCCCCCCCOC(=O)CC12CC3CC(CC(C3)C1)C2)OC(=O)CCCN(C)C] |
| C30 | 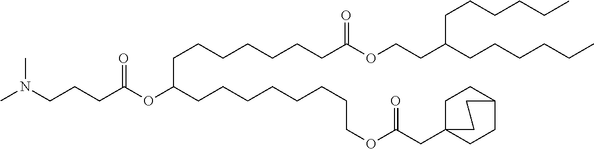 [SMILES: CCCCCCC(CCCCCC)CCOC(=O)CCCCCCCC(CCCCCCCCOC(=O)CC12CCC(CC1)CC2)OC(=O)CCCN(C)C] |
| C31 | 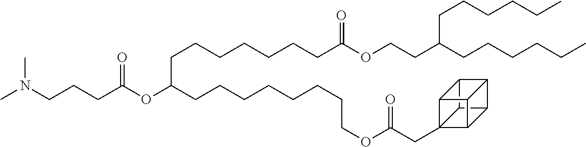 [SMILES: CCCCCCC(CCCCCC)CCOC(=O)CCCCCCCC(CCCCCCCCOC(=O)CC12C3C4C5C3C1C5C42)OC(=O)CCCN(C)C] |
| C32 | 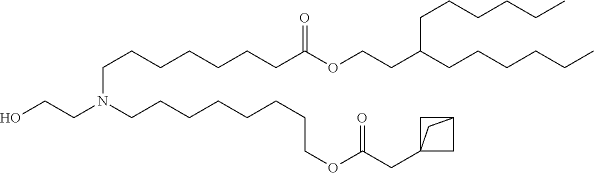 [SMILES: CCCCCCC(CCCCCC)CCOC(=O)CCCCCCCN(CCO)CCCCCCCCOC(=O)CC12CC(C1)C2] |
| C33 | 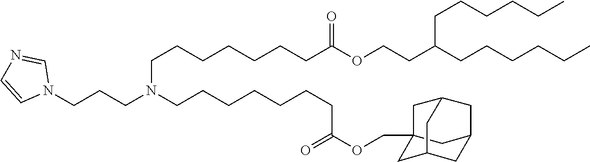 [SMILES: CCCCCCC(CCCCCC)CCOC(=O)CCCCCCCN(CCCCCCCC(=O)OCC12CC3CC(CC(C3)C1)C2)CCCn1ccnc1] |
| C34 | 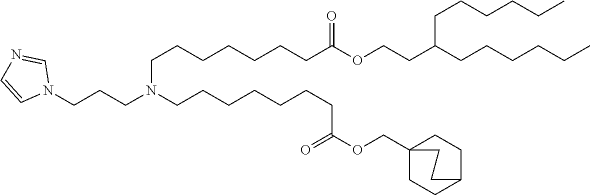 [SMILES: CCCCCCC(CCCCCC)CCOC(=O)CCCCCCCN(CCCCCCCC(=O)OCC12CCC(CC1)CC2)CCCn1ccnc1] |
| C35 | 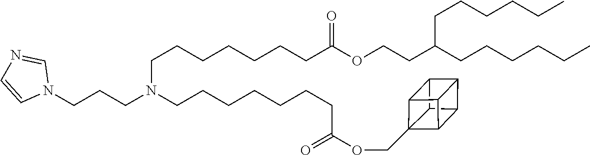 [SMILES: CCCCCCC(CCCCCC)CCOC(=O)CCCCCCCN(CCCCCCCC(=O)OCC12C3C4C5C3C1C5C42)CCCn1ccnc1] |
| C36 | 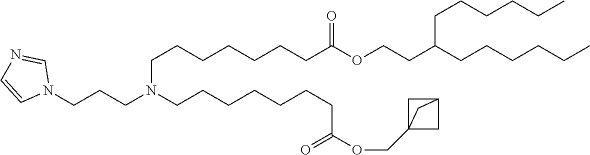 [SMILES: CCCCCCC(CCCCCC)CCOC(=O)CCCCCCCN(CCCCCCCC(=O)OCC12CC(C1)C2)CCCn1ccnc1] |
| C37 | 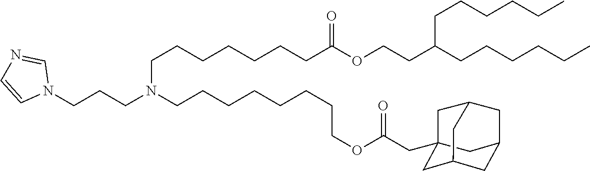 [SMILES: CCCCCCC(CCCCCC)CCOC(=O)CCCCCCCN(CCCCCCCCOC(=O)CC12CC3CC(CC(C3)C1)C2)CCCn1ccnc1] |
| C38 | 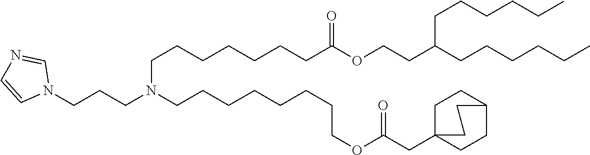 [SMILES: CCCCCCC(CCCCCC)CCOC(=O)CCCCCCCN(CCCCCCCCOC(=O)CC12CCC(CC1)CC2)CCCn1ccnc1] |
| C39 | 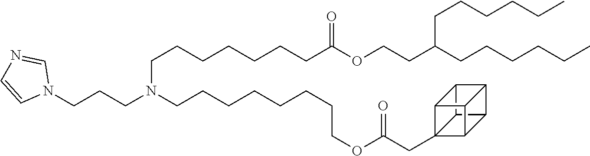 [SMILES: CCCCCCC(CCCCCC)CCOC(=O)CCCCCCCN(CCCCCCCCOC(=O)CC12C3C4C5C3C1C5C42)CCCn1ccnc1] |
| C40 | 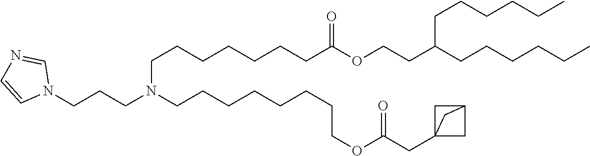 [SMILES: CCCCCCC(CCCCCC)CCOC(=O)CCCCCCCN(CCCCCCCCOC(=O)CC12CC(C1)C2)CCCn1ccnc1] |
| C41 | 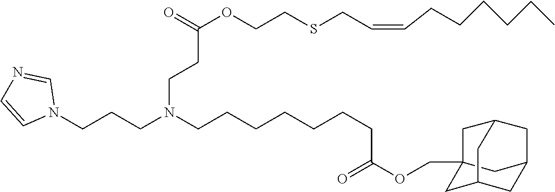 [SMILES: CCCCCC/C=C\CSCCOC(=O)CCN(CCCCCCCC(=O)OCC12CC3CC(CC(C3)C1)C2)CCCn1ccnc1] |
| C42 | 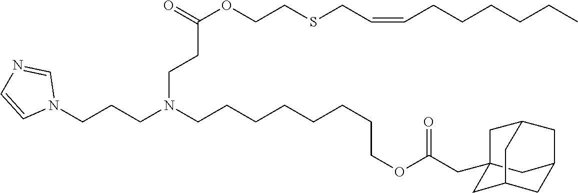 [SMILES: CCCCCC/C=C\CSCCOC(=O)CCN(CCCCCCCCOC(=O)CC12CC3CC(CC(C3)C1)C2)CCCn1ccnc1] |
| C43 | 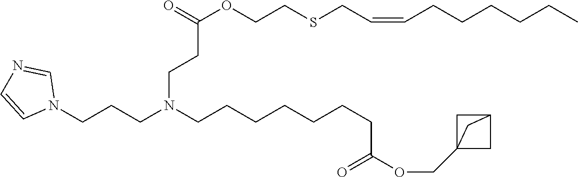 [SMILES: CCCCCC/C=C\CSCCOC(=O)CCN(CCCCCCCC(=O)OCC12CC(C1)C2)CCCn1ccnc1] |
| C44 | 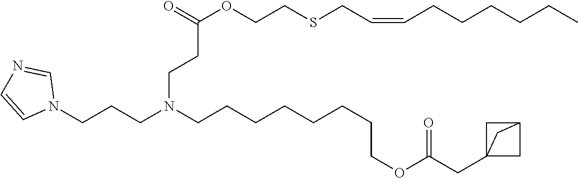 [SMILES: CCCCCC/C=C\CSCCOC(=O)CCN(CCCCCCCCOC(=O)CC12CC(C1)C2)CCCn1ccnc1] |
| C45 | 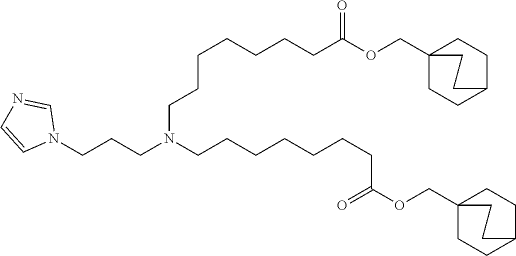 [SMILES: O=C(CCCCCCCN(CCCCCCCC(=O)OCC12CCC(CC1)CC2)CCCn1ccnc1)OCC12CCC(CC1)CC2] |
| C46 | 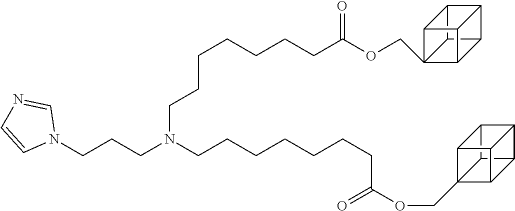 [SMILES: O=C(CCCCCCCN(CCCCCCCC(=O)OCC12C3C4C5C3C1C5C42)CCCn1ccnc1)OCC12C3C4C5C3C1C5C42] |
| C47 | 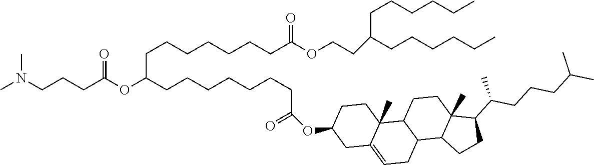 |
| C48 | 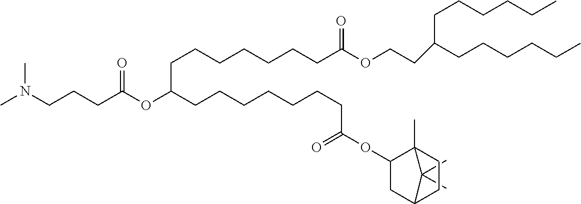 [SMILES: CCCCCCC(CCCCCC)CCOC(=O)CCCCCCCC(CCCCCCCC(=O)OC1CC2CCC1(C)C2(C)C)OC(=O)CCCN(C)C] |
| C49 | 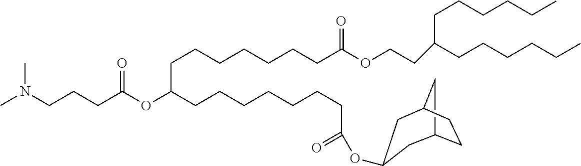 [SMILES: CCCCCCC(CCCCCC)CCOC(=O)CCCCCCCC(CCCCCCCC(=O)OC1CC2CCC(C2)C1)OC(=O)CCCN(C)C] |
| C50 | 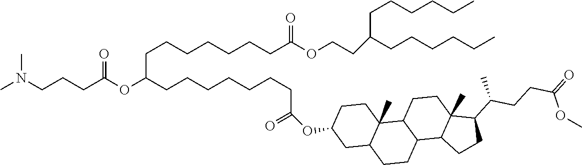 |
| C51 | 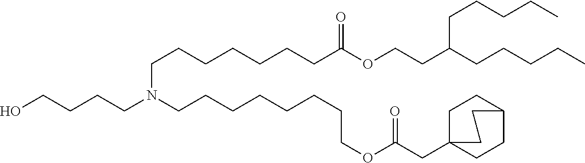 [SMILES: CCCCCC(CCCCC)CCOC(=O)CCCCCCCN(CCCCO)CCCCCCCCOC(=O)CC12CCC(CC1)CC2] |
| C52 | 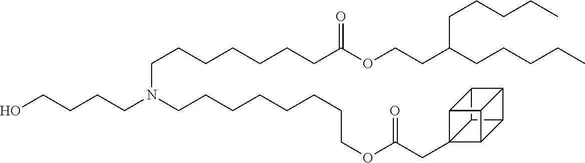 [SMILES: CCCCCC(CCCCC)CCOC(=O)CCCCCCCN(CCCCO)CCCCCCCCOC(=O)CC12C3C4C5C3C1C5C42] |
| C53 | 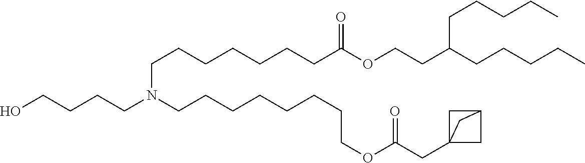 [SMILES: CCCCCC(CCCCC)CCOC(=O)CCCCCCCN(CCCCO)CCCCCCCCOC(=O)CC12CC(C1)C2] |
| C54 | 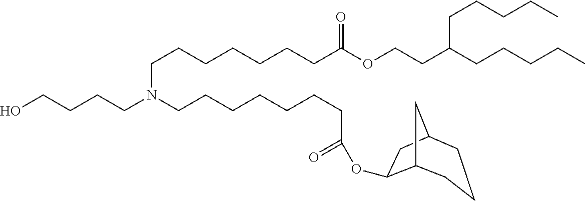 [SMILES: CCCCCC(CCCCC)CCOC(=O)CCCCCCCN(CCCCO)CCCCCCCC(=O)OC1CC2CCCC1C2] |
| C55 | 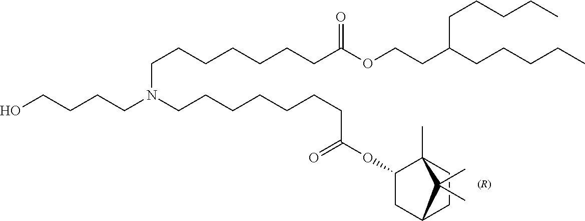 |
| C56 | 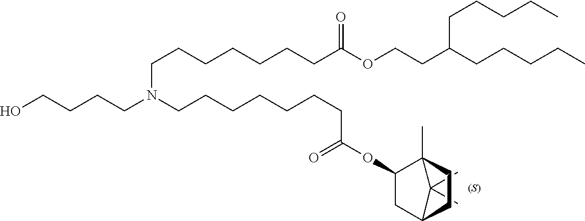 |
| C57 | 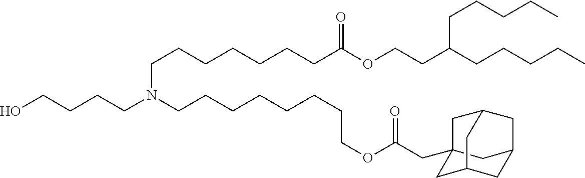 [SMILES: CCCCCC(CCCCC)CCOC(=O)CCCCCCCN(CCCCO)CCCCCCCCOC(=O)CC12CC3CC(CC(C3)C1)C2] |
| C58 | 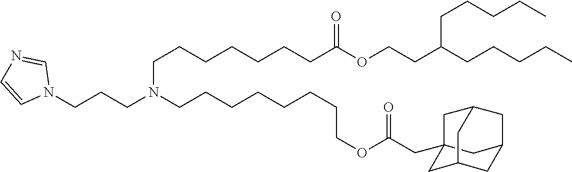 [SMILES: CCCCCC(CCCCC)CCOC(=O)CCCCCCCN(CCCCCCCCOC(=O)CC12CC3CC(CC(C3)C1)C2)CCCn1ccnc1] |
| C59 | 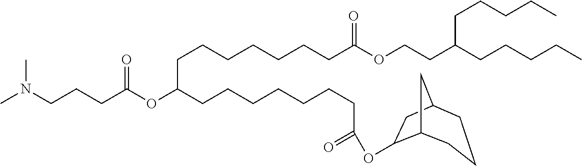 [SMILES: CCCCCC(CCCCC)CCOC(=O)CCCCCCCC(CCCCCCCC(=O)OC1CC2CCCC1C2)OC(=O)CCCN(C)C] |
| C60 | 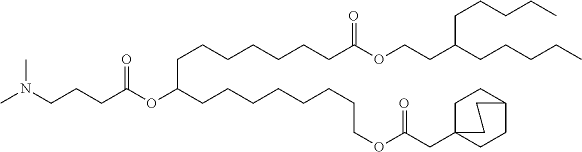 [SMILES: CCCCCC(CCCCC)CCOC(=O)CCCCCCCC(CCCCCCCCOC(=O)CC12CCC(CC1)CC2)OC(=O)CCCN(C)C] |
| C61 | 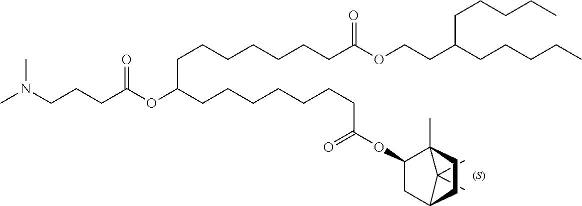 |
| C62 | 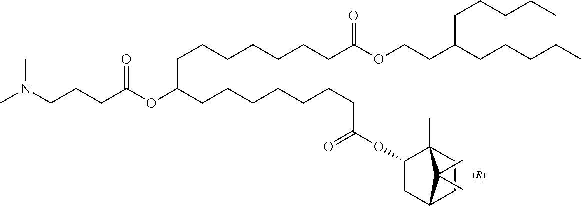 |
| C63 | 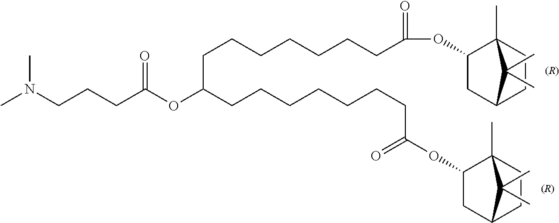 |
| C64 | 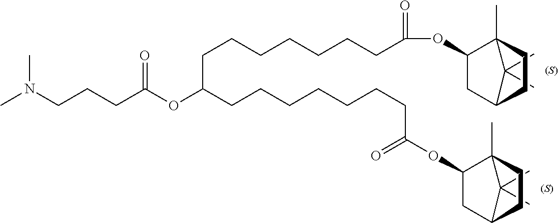 |
| C65 | 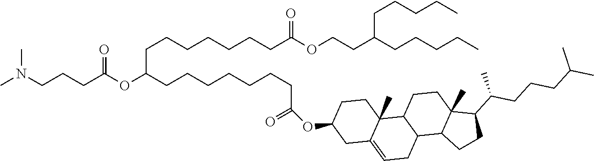 |
| C66 | 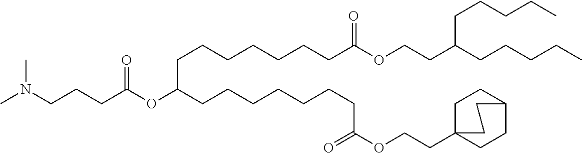 [SMILES: CCCCCC(CCCCC)CCOC(=O)CCCCCCCC(CCCCCCCC(=O)OCCC12CCC(CC1)CC2)OC(=O)CCCN(C)C] |
| C67 | 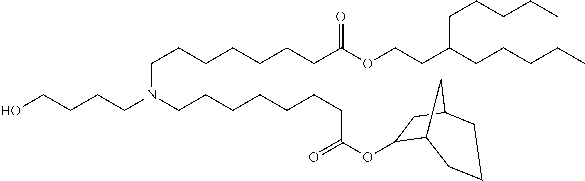 [SMILES: CCCCCC(CCCCC)CCOC(=O)CCCCCCCN(CCCCO)CCCCCCCC(=O)OC1CC2CCCC1C2] |
| C68 | 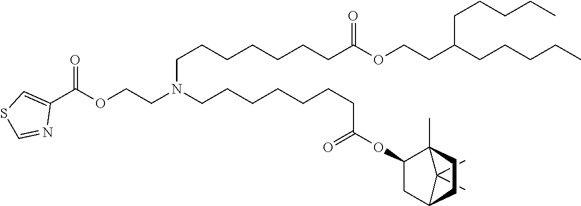 |
| C69 | 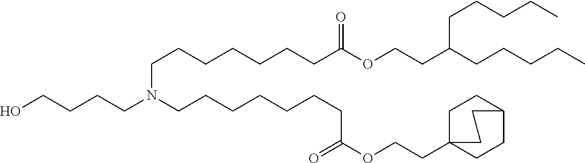 [SMILES: CCCCCC(CCCCC)CCOC(=O)CCCCCCCN(CCCCO)CCCCCCCC(=O)OCCC12CCC(CC1)CC2] |
| C70 | 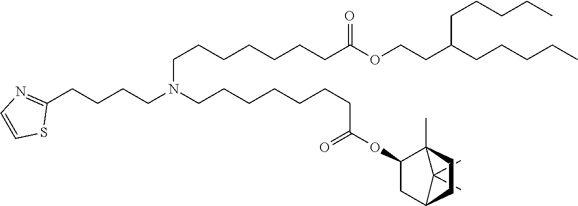 |
| C71 | 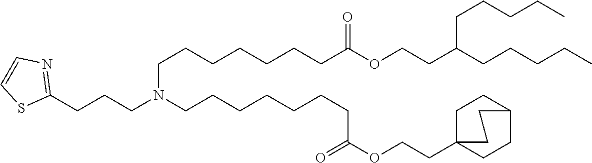 [SMILES: CCCCCC(CCCCC)CCOC(=O)CCCCCCCN(CCCCCCCC(=O)OCCC12CCC(CC1)CC2)CCCc1nccs1] |
| C72 | 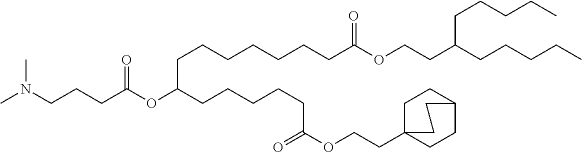 [SMILES: CCCCCC(CCCCC)CCOC(=O)CCCCCCCC(CCCCCC(=O)OCCC12CCC(CC1)CC2)OC(=O)CCCN(C)C] |
| C73 | 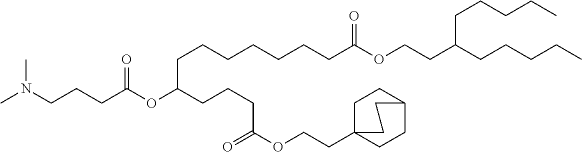 [SMILES: CCCCCC(CCCCC)CCOC(=O)CCCCCCCC(CCCC(=O)OCCC12CCC(CC1)CC2)OC(=O)CCCN(C)C] |
| C74 | 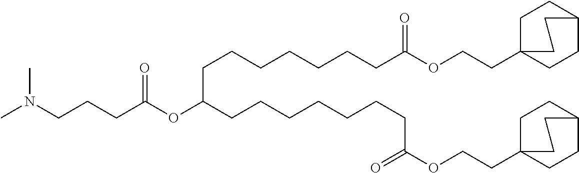 [SMILES: CN(C)CCCC(=O)OC(CCCCCCCC(=O)OCCC12CCC(CC1)CC2)CCCCCCCC(=O)OCCC12CCC(CC1)CC2] |
| C75 | 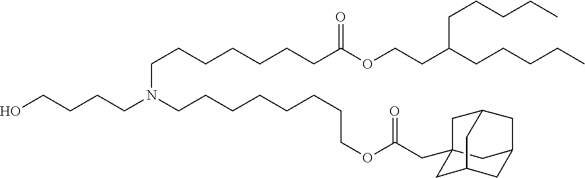 [SMILES: CCCCCC(CCCCC)CCOC(=O)CCCCCCCN(CCCCO)CCCCCCCCOC(=O)CC12CC3CC(CC(C3)C1)C2] |
| C76 | 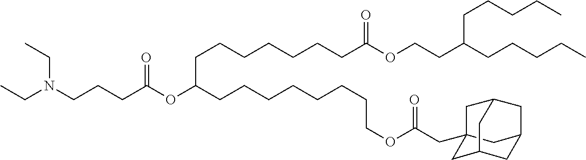 [SMILES: CCCCCC(CCCCC)CCOC(=O)CCCCCCCC(CCCCCCCCOC(=O)CC12CC3CC(CC(C3)C1)C2)OC(=O)CCCN(CC)CC] |
| C77 | 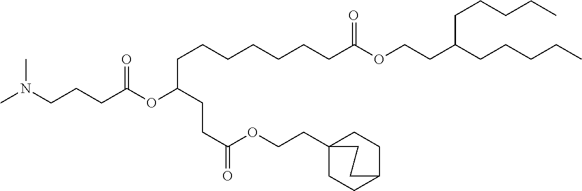 [SMILES: CCCCCC(CCCCC)CCOC(=O)CCCCCCCC(CCC(=O)OCCC12CCC(CC1)CC2)OC(=O)CCCN(C)C] |
| C78 | 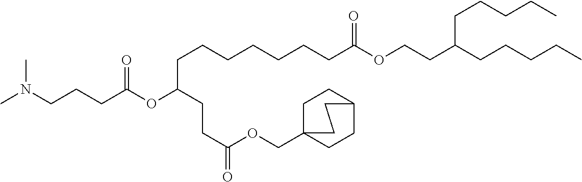 [SMILES: CCCCCC(CCCCC)CCOC(=O)CCCCCCCC(CCC(=O)OCC12CCC(CC1)CC2)OC(=O)CCCN(C)C] |
| C79 | 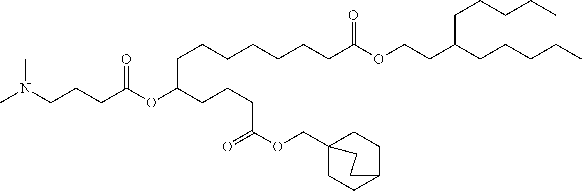 [SMILES: CCCCCC(CCCCC)CCOC(=O)CCCCCCCC(CCCC(=O)OCC12CCC(CC1)CC2)OC(=O)CCCN(C)C] |
| C80 | 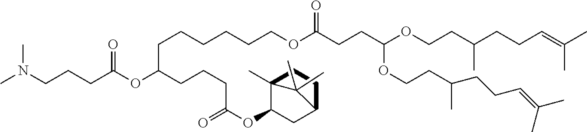 |
| C81 | 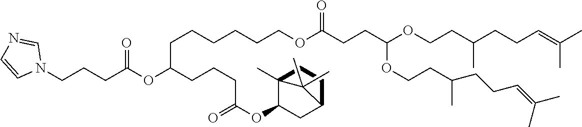 |
| C82 | 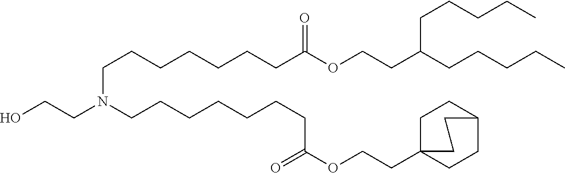 [SMILES: CCCCCC(CCCCC)CCOC(=O)CCCCCCCN(CCO)CCCCCCCC(=O)OCCC12CCC(CC1)CC2] |
| C83 | 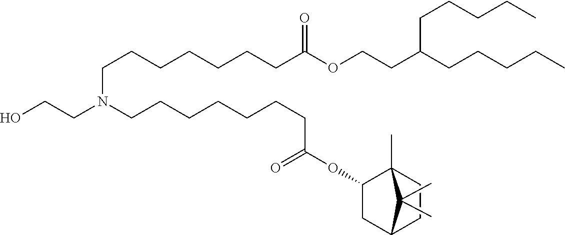 [SMILES: CCCCCC(CCCCC)CCOC(=O)CCCCCCCN(CCO)CCCCCCCC(=O)O[C@H]1C[C@H]2CC[C@]1(C)C2(C)C] |
| C84 | 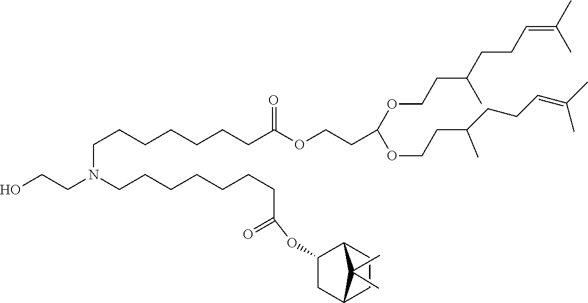 [SMILES: CC(C)=CCCC(C)CCOC(CCOC(=O)CCCCCCCN(CCO)CCCCCCCC(=O)O[C@H]1C[C@H]2CC[C@@H]1C2(C)C)OCCC(C)CCC=C(C)C] |
| C85 | 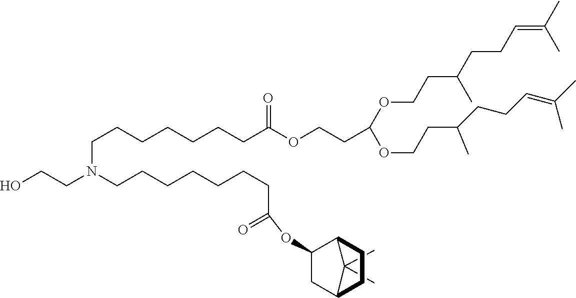 |
| C86 | 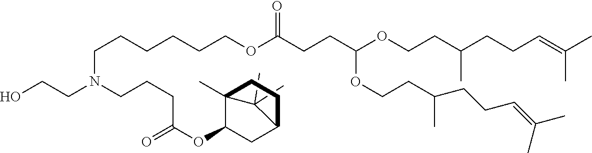 |
| C87 | 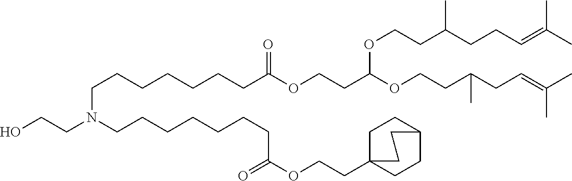 [SMILES: CC(C)=CCCC(C)CCOC(CCOC(=O)CCCCCCCN(CCO)CCCCCCCC(=O)OCCC12CCC(CC1)CC2)OCCC(C)CC=C(C)C] |
| C88 | 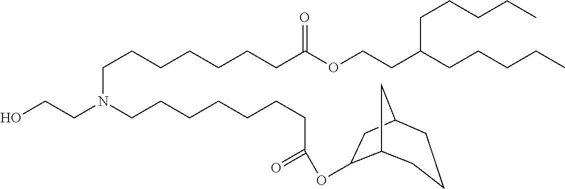 [SMILES: CCCCCC(CCCCC)CCOC(=O)CCCCCCCN(CCO)CCCCCCCC(=O)OC1CC2CCCC1C2] |
| C89 | 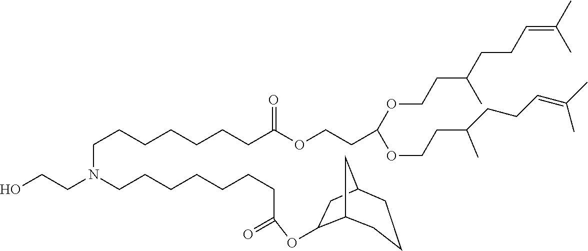 [SMILES: CC(C)=CCCC(C)CCOC(CCOC(=O)CCCCCCCN(CCO)CCCCCCCC(=O)OC1CC2CCCC1C2)OCCC(C)CCC=C(C)C] |
| C90 | 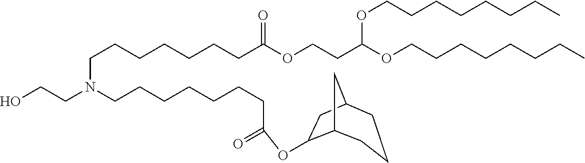 [SMILES: CCCCCCCCOC(CCOC(=O)CCCCCCCN(CCO)CCCCCCCC(=O)OC1CC2CCCC1C2)OCCCCCCCC] |
| C91 | 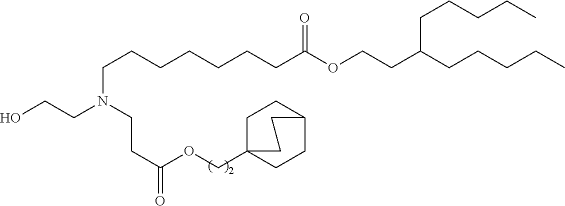 |
| C92 | 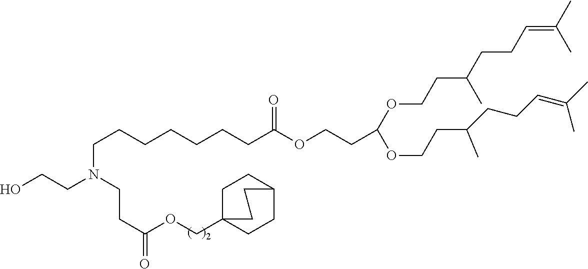 |
| C93 | 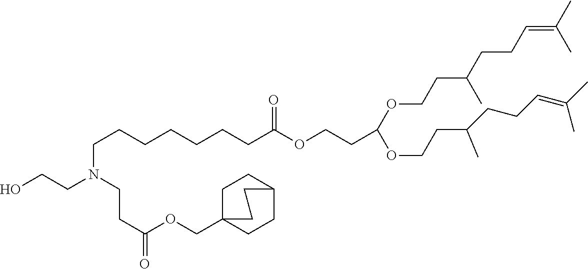 [SMILES: CC(C)=CCCC(C)CCOC(CCOC(=O)CCCCCCCN(CCO)CCC(=O)OCC12CCC(CC1)CC2)OCCC(C)CCC=C(C)C] |

### Series “CX”

In some embodiments, an LNP of the present disclosure comprises an ionizable lipid disclosed in PCT Publication WO2023196931A1, which is incorporated by reference herein, in its entirety.

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CX-I):

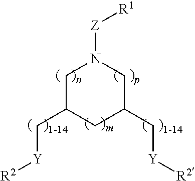

or a pharmaceutically acceptable salt thereof, wherein
Z is selected from the group consisting of a bond,

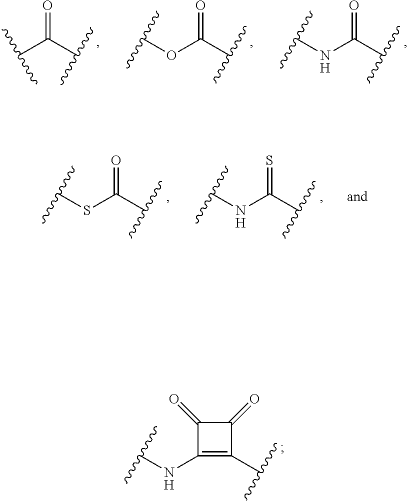

each Y is independently selected from the group consisting of

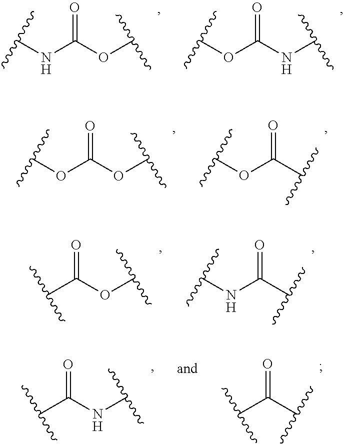

R¹ is —(CH₂)₁₋₆N(R^{a})₂ or —(CH₂)₁₋₆OH;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-i):

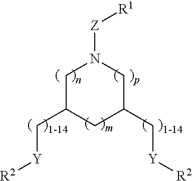

or a pharmaceutically acceptable salt thereof, wherein
Z is selected from the group consisting of a bond,

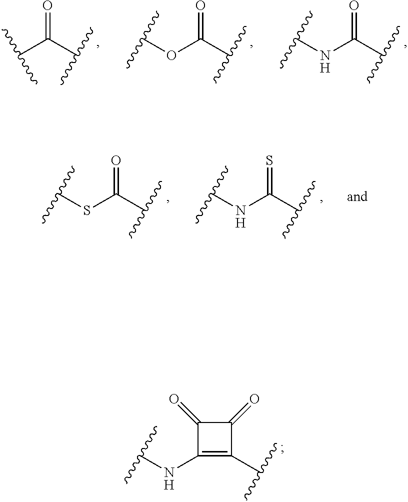

each Y is independently selected from the group consisting of H

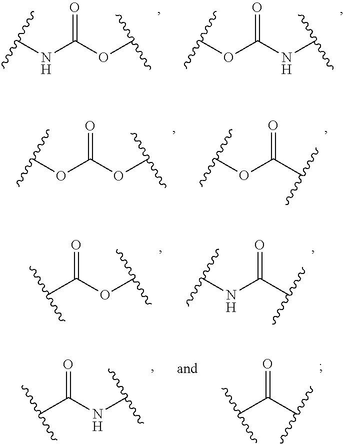

R¹ is —(CH₂)₁₋₆N(R^{a})₂; R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure comprises a compound selected from any lipid in Table (I-D) below or a pharmaceutically acceptable salt thereof:

TABLE (I-D)
Non-Limiting Examples of Ionizable Lipids

| Structure | Cmpd # |
|---|---|
| 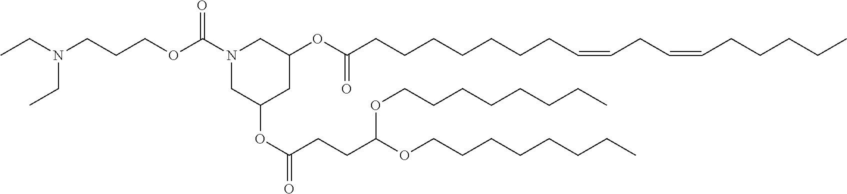 [SMILES: CCCCC/C=C\C/C=C\CCCCCCCC(=O)OC1CC(OC(=O)CCC(OCCCCCCCC)OCCCCCCCC)CN(C(=O)OCCCN(CC)CC)C1] | CX-1 |
| 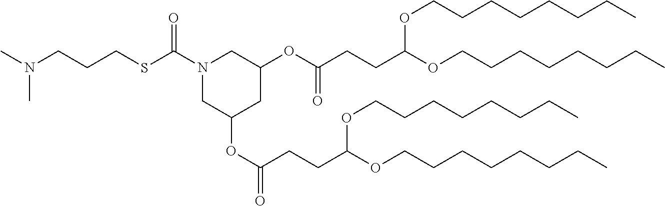 [SMILES: CCCCCCCCOC(CCC(=O)OC1CC(OC(=O)CCC(OCCCCCCCC)OCCCCCCCC)CN(C(=O)SCCCN(C)C)C1)OCCCCCCCC] | CX-2 |
| 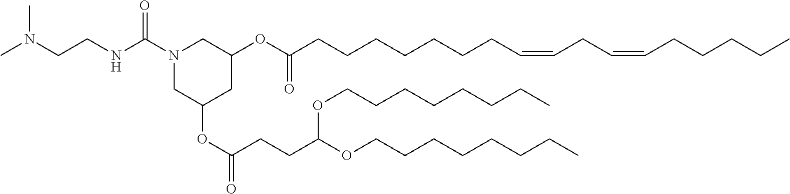 [SMILES: CCCCC/C=C\C/C=C\CCCCCCCC(=O)OC1CC(OC(=O)CCC(OCCCCCCCC)OCCCCCCCC)CN(C(=O)NCCN(C)C)C1] | CX-3 |
| 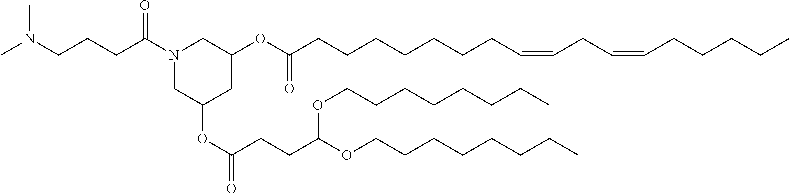 [SMILES: CCCCC/C=C\C/C=C\CCCCCCCC(=O)OC1CC(OC(=O)CCC(OCCCCCCCC)OCCCCCCCC)CN(C(=O)CCCN(C)C)C1] | CX-4 |
| 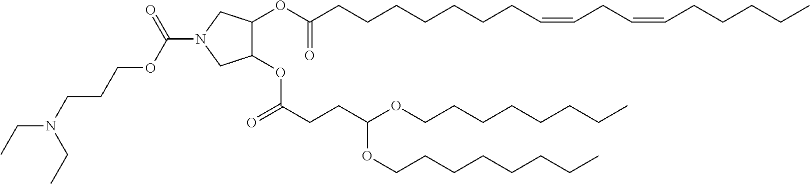 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-5 |
| 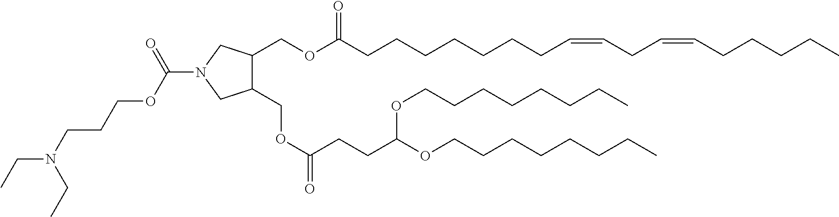 [SMILES: CCCCC/C=C\C/C=C\CCCCCCCC(=O)OCC1CN(C(=O)OCCCN(CC)CC)CC1COC(=O)CCC(OCCCCCCCC)OCCCCCCCC] | CX-6 |
| 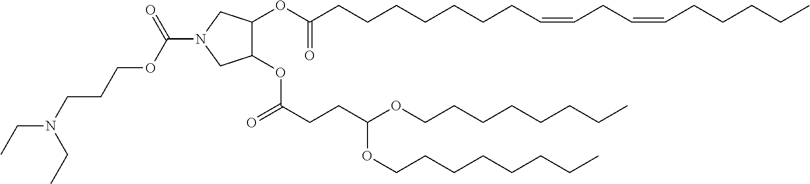 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-7 |
| 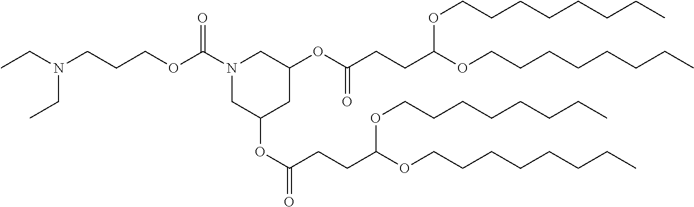 [SMILES: CCCCCCCCOC(CCC(=O)OC1CC(OC(=O)CCC(OCCCCCCCC)OCCCCCCCC)CN(C(=O)OCCCN(CC)CC)C1)OCCCCCCCC] | CX-8 |
| 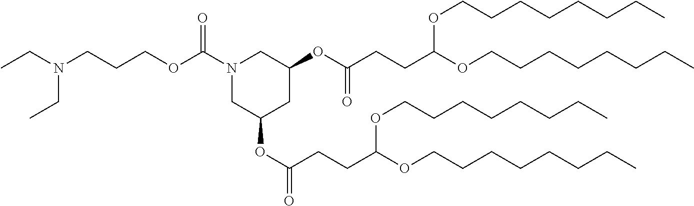 [SMILES: CCCCCCCCOC(CCC(=O)O[C@@H]1C[C@H](OC(=O)CCC(OCCCCCCCC)OCCCCCCCC)CN(C(=O)OCCCN(CC)CC)C1)OCCCCCCCC] | CX-8a |
| 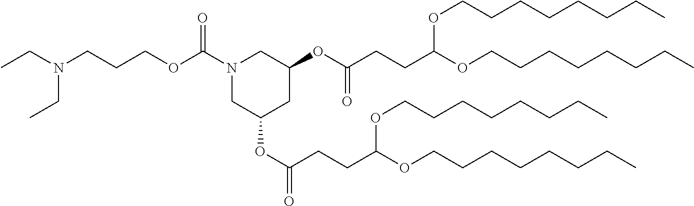 [SMILES: CCCCCCCCOC(CCC(=O)O[C@H]1C[C@H](OC(=O)CCC(OCCCCCCCC)OCCCCCCCC)CN(C(=O)OCCCN(CC)CC)C1)OCCCCCCCC] | CX-8b |
| 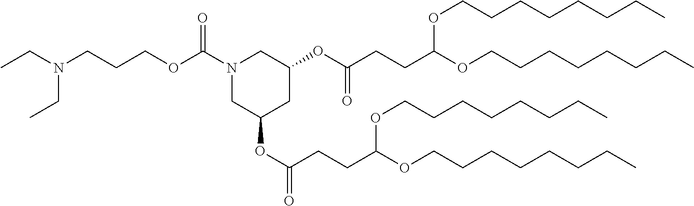 [SMILES: CCCCCCCCOC(CCC(=O)O[C@@H]1C[C@@H](OC(=O)CCC(OCCCCCCCC)OCCCCCCCC)CN(C(=O)OCCCN(CC)CC)C1)OCCCCCCCC] | CX-8c |
| 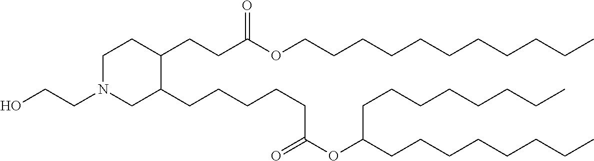 [SMILES: CCCCCCCCCCCOC(=O)CCC1CCN(CCO)CC1CCCCCC(=O)OC(CCCCCCCC)CCCCCCCC] | CX-9 |
| 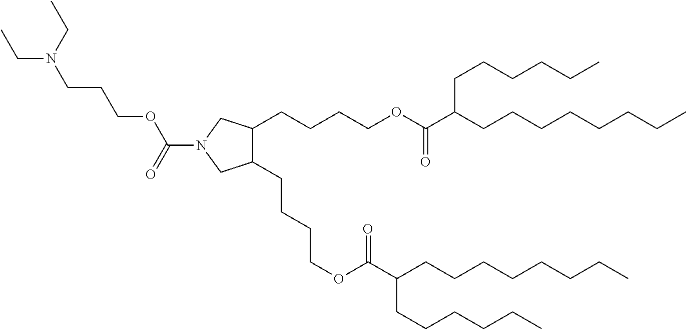 [SMILES: CCCCCCCCC(CCCCCC)C(=O)OCCCCC1CN(C(=O)OCCCN(CC)CC)CC1CCCCOC(=O)C(CCCCCC)CCCCCCCC] | CX-10 |
| 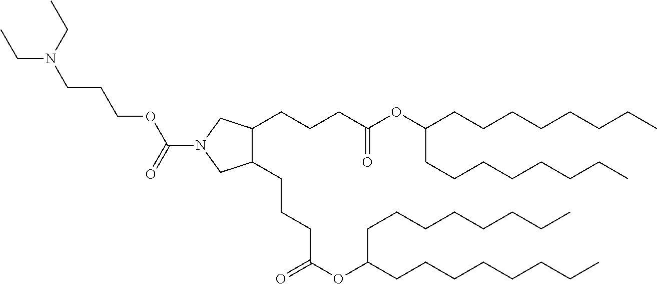 [SMILES: CCCCCCCCC(CCCCCCCC)OC(=O)CCCC1CN(C(=O)OCCCN(CC)CC)CC1CCCC(=O)OC(CCCCCCCC)CCCCCCCC] | CX-11 |
| 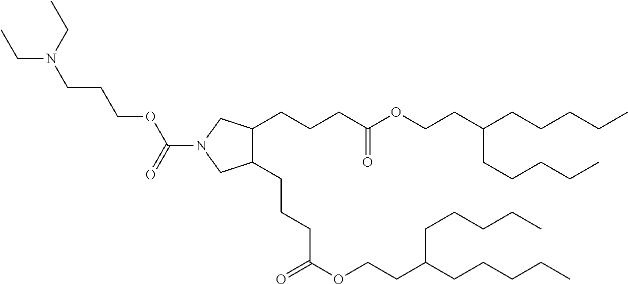 [SMILES: CCCCCC(CCCCC)CCOC(=O)CCCC1CN(C(=O)OCCCN(CC)CC)CC1CCCC(=O)OCCC(CCCCC)CCCCC] | CX-12 |
| 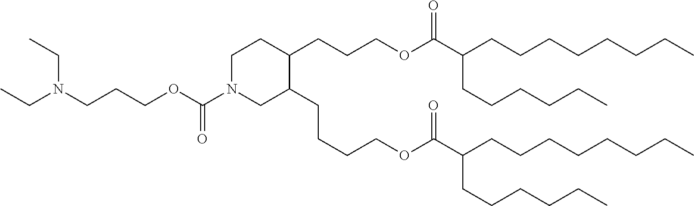 [SMILES: CCCCCCCCC(CCCCCC)C(=O)OCCCCC1CN(C(=O)OCCCN(CC)CC)CCC1CCCOC(=O)C(CCCCCC)CCCCCCCC] | CX-13 |
| 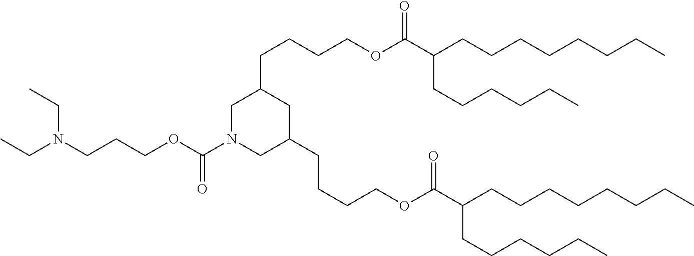 [SMILES: CCCCCCCCC(CCCCCC)C(=O)OCCCCC1CC(CCCCOC(=O)C(CCCCCC)CCCCCCCC)CN(C(=O)OCCCN(CC)CC)C1] | CX-14 |
| 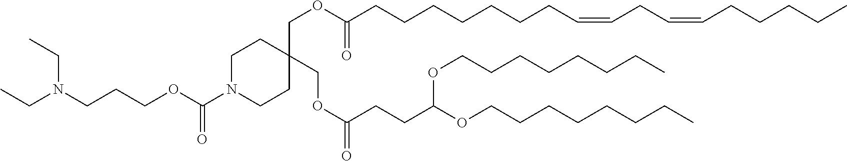 [SMILES: CCCCC/C=C\C/C=C\CCCCCCCC(=O)OCC1(COC(=O)CCC(OCCCCCCCC)OCCCCCCCC)CCN(C(=O)OCCCN(CC)CC)CC1] | CX-15 |
| 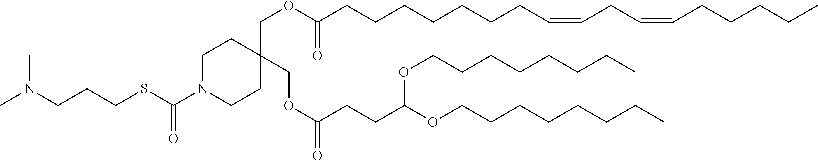 [SMILES: CCCCC/C=C\C/C=C\CCCCCCCC(=O)OCC1(COC(=O)CCC(OCCCCCCCC)OCCCCCCCC)CCN(C(=O)SCCCN(C)C)CC1] | CX-16 |
| 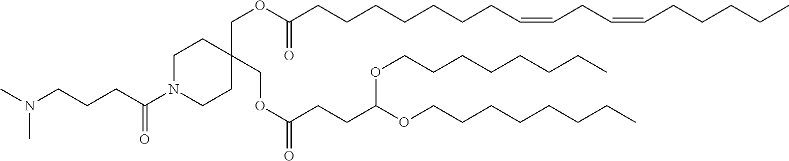 [SMILES: CCCCC/C=C\C/C=C\CCCCCCCC(=O)OCC1(COC(=O)CCC(OCCCCCCCC)OCCCCCCCC)CCN(C(=O)CCCN(C)C)CC1] | CX-17 |
| 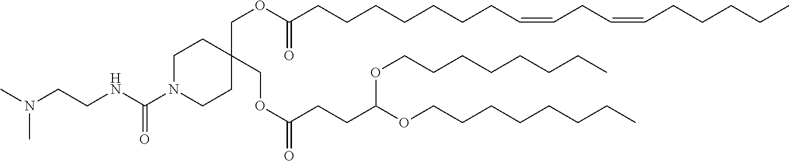 [SMILES: CCCCC/C=C\C/C=C\CCCCCCCC(=O)OCC1(COC(=O)CCC(OCCCCCCCC)OCCCCCCCC)CCN(C(=O)NCCN(C)C)CC1] | CX-18 |
| 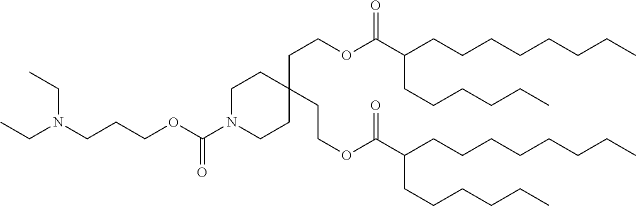 [SMILES: CCCCCCCCC(CCCCCC)C(=O)OCCC1(CCOC(=O)C(CCCCCC)CCCCCCCC)CCN(C(=O)OCCCN(CC)CC)CC1] | CX-19 |
| 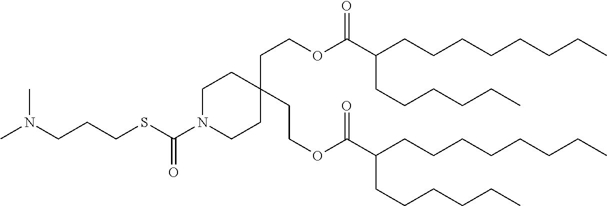 [SMILES: CCCCCCCCC(CCCCCC)C(=O)OCCC1(CCOC(=O)C(CCCCCC)CCCCCCCC)CCN(C(=O)SCCCN(C)C)CC1] | CX-20 |
| 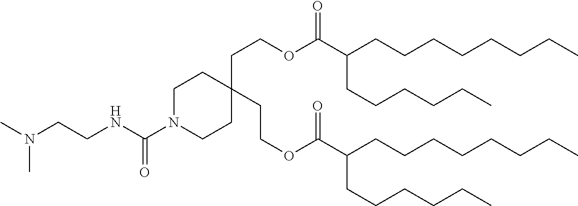 [SMILES: CCCCCCCCC(CCCCCC)C(=O)OCCC1(CCOC(=O)C(CCCCCC)CCCCCCCC)CCN(C(=O)NCCN(C)C)CC1] | CX-21 |
| 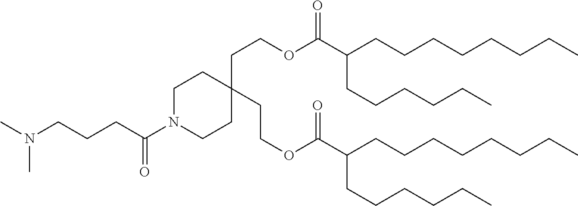 [SMILES: CCCCCCCCC(CCCCCC)C(=O)OCCC1(CCOC(=O)C(CCCCCC)CCCCCCCC)CCN(C(=O)CCCN(C)C)CC1] | CX-22 |
| 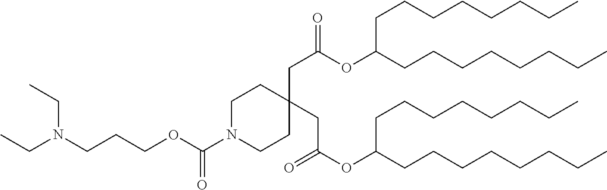 [SMILES: CCCCCCCCC(CCCCCCCC)OC(=O)CC1(CC(=O)OC(CCCCCCCC)CCCCCCCC)CCN(C(=O)OCCCN(CC)CC)CC1] | CX-23 |
| 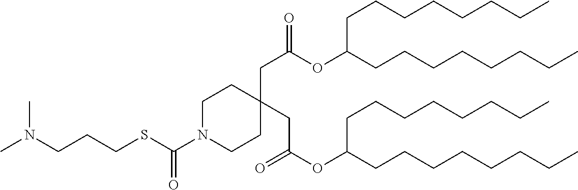 [SMILES: CCCCCCCCC(CCCCCCCC)OC(=O)CC1(CC(=O)OC(CCCCCCCC)CCCCCCCC)CCN(C(=O)SCCCN(C)C)CC1] | CX-24 |
| 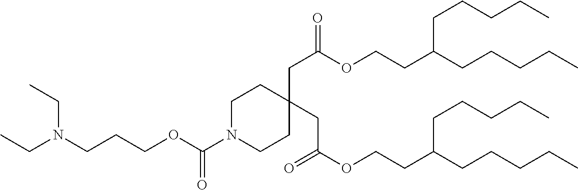 [SMILES: CCCCCC(CCCCC)CCOC(=O)CC1(CC(=O)OCCC(CCCCC)CCCCC)CCN(C(=O)OCCCN(CC)CC)CC1] | CX-25 |
| 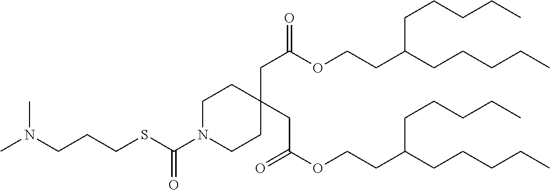 [SMILES: CCCCCC(CCCCC)CCOC(=O)CC1(CC(=O)OCCC(CCCCC)CCCCC)CCN(C(=O)SCCCN(C)C)CC1] | CX-26 |
| 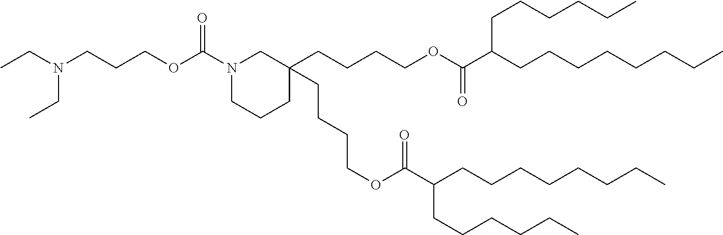 [SMILES: CCCCCCCCC(CCCCCC)C(=O)OCCCCC1(CCCCOC(=O)C(CCCCCC)CCCCCCCC)CCCN(C(=O)OCCCN(CC)CC)C1] | CX-27 |
| 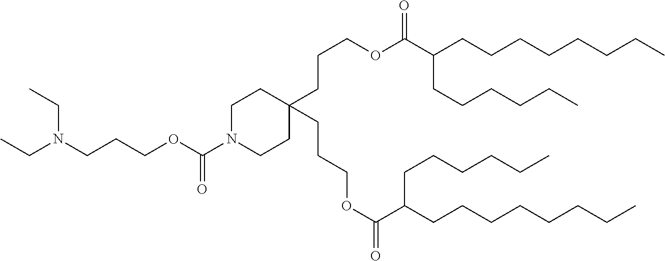 [SMILES: CCCCCCCCC(CCCCCC)C(=O)OCCCC1(CCCOC(=O)C(CCCCCC)CCCCCCCC)CCN(C(=O)OCCCN(CC)CC)CC1] | CX-28 |
| 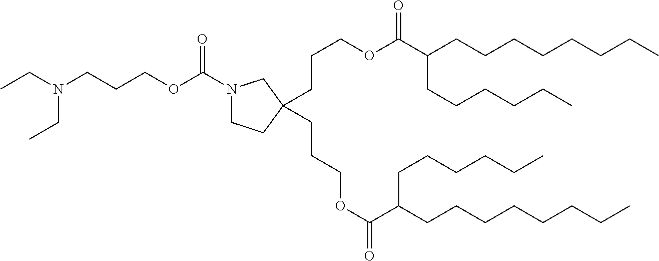 [SMILES: CCCCCCCCC(CCCCCC)C(=O)OCCCC1(CCCOC(=O)C(CCCCCC)CCCCCCCC)CCN(C(=O)OCCCN(CC)CC)C1] | CX-29 |
| 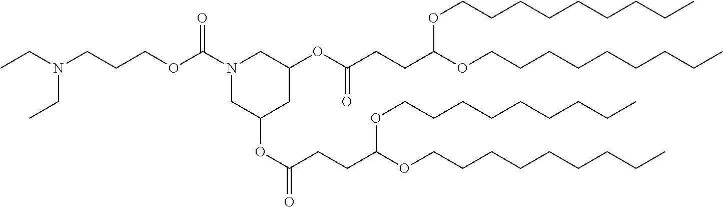 [SMILES: CCCCCCCCCOC(CCC(=O)OC1CC(OC(=O)CCC(OCCCCCCCCC)OCCCCCCCCC)CN(C(=O)OCCCN(CC)CC)C1)OCCCCCCCCC] | CX-30 |
| 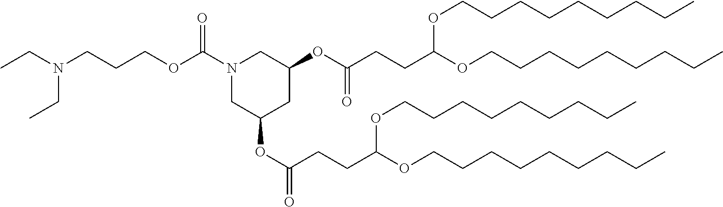 [SMILES: CCCCCCCCCOC(CCC(=O)O[C@@H]1C[C@H](OC(=O)CCC(OCCCCCCCCC)OCCCCCCCCC)CN(C(=O)OCCCN(CC)CC)C1)OCCCCCCCCC] | CX-30a |
| 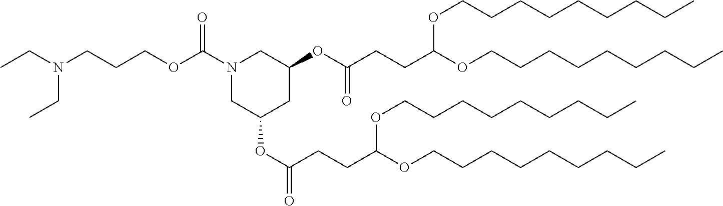 [SMILES: CCCCCCCCCOC(CCC(=O)O[C@H]1C[C@H](OC(=O)CCC(OCCCCCCCCC)OCCCCCCCCC)CN(C(=O)OCCCN(CC)CC)C1)OCCCCCCCCC] | CX-30b |
| 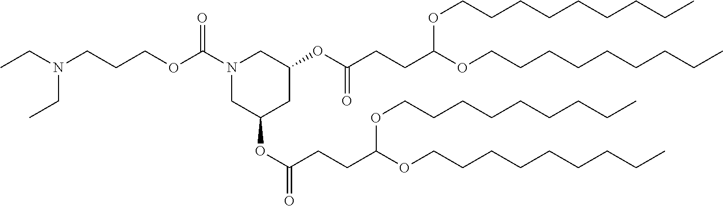 [SMILES: CCCCCCCCCOC(CCC(=O)O[C@@H]1C[C@@H](OC(=O)CCC(OCCCCCCCCC)OCCCCCCCCC)CN(C(=O)OCCCN(CC)CC)C1)OCCCCCCCCC] | CX-30c |

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

### Series “CZ”

In some embodiments, an LNP of the present disclosure comprises an ionizable lipid disclosed in PCT Publication WO2023196931A1, which is incorporated by reference herein, in its entirety.

In some embodiments, a compound of the present disclosure is represented by Formula (CZ-I)

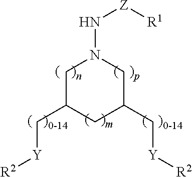

or a pharmaceutically acceptable salt thereof, wherein
Z is selected from the group consisting of a bond,

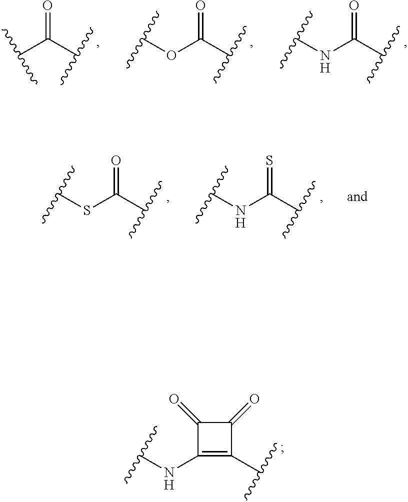

each Y is independently selected from the group consisting of,

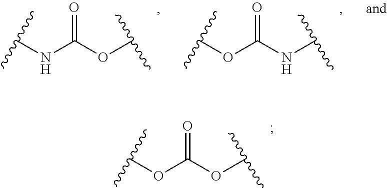 [SMILES: *NC(=O)O* ; *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure comprises a compound selected from any lipid in Table (I-E) below or a pharmaceutically acceptable salt thereof:

TABLE (I-E)
Non-Limiting Examples of Ionizable Lipids

| | Compound |
|---|---|
| Structure | No. |
| 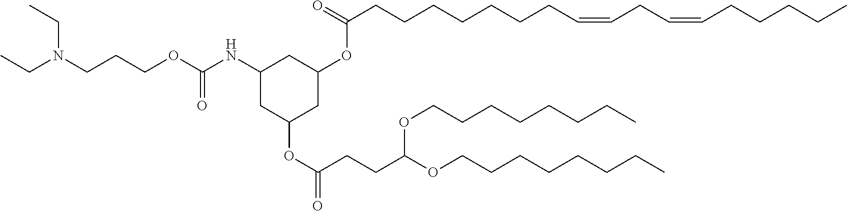 [SMILES: CCCCC/C=C\C/C=C\CCCCCCCC(=O)OC1CC(NC(=O)OCCCN(CC)CC)CC(OC(=O)CCC(OCCCCCCCC)OCCCCCCCC)C1] | CZ-1 |
| 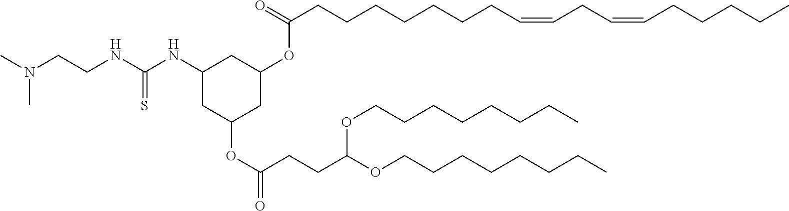 [SMILES: CCCCC/C=C\C/C=C\CCCCCCCC(=O)OC1CC(NC(=S)NCCN(C)C)CC(OC(=O)CCC(OCCCCCCCC)OCCCCCCCC)C1] | CZ-2 |
| 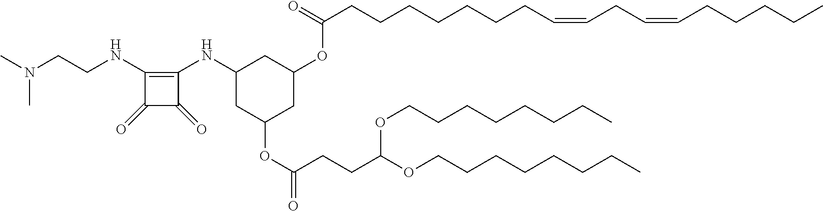 [SMILES: CCCCC/C=C\C/C=C\CCCCCCCC(=O)OC1CC(Nc2c(NCCN(C)C)c(=O)c2=O)CC(OC(=O)CCC(OCCCCCCCC)OCCCCCCCC)C1] | CZ-3 |
| 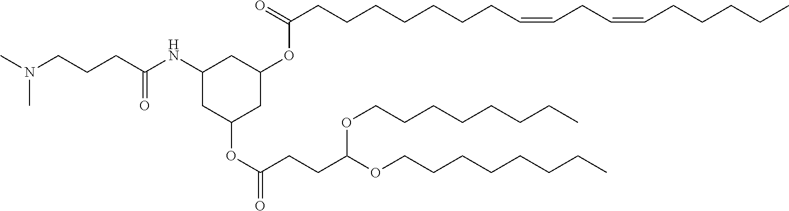 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-4 |
| 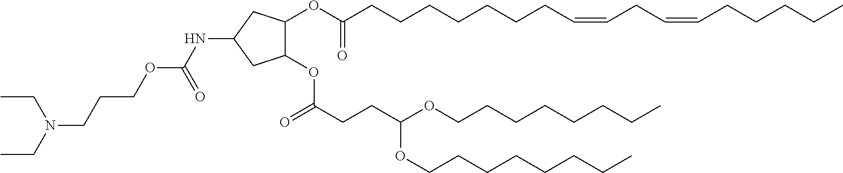 [SMILES: CCCCC/C=C\C/C=C\CCCCCCCC(=O)OC1CC(NC(=O)OCCCN(CC)CC)CC1OC(=O)CCC(OCCCCCCCC)OCCCCCCCC] | CZ-5 |
| 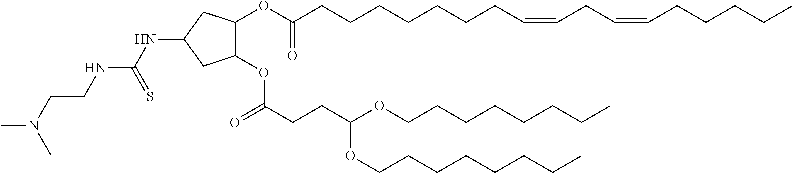 [SMILES: CCCCC/C=C\C/C=C\CCCCCCCC(=O)OC1CC(NC(=S)NCCN(C)C)CC1OC(=O)CCC(OCCCCCCCC)OCCCCCCCC] | CZ-6 |
| 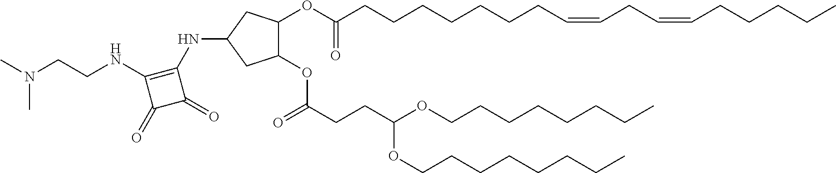 [SMILES: CCCCC/C=C\C/C=C\CCCCCCCC(=O)OC1CC(Nc2c(NCCN(C)C)c(=O)c2=O)CC1OC(=O)CCC(OCCCCCCCC)OCCCCCCCC] | CZ-7 |
| 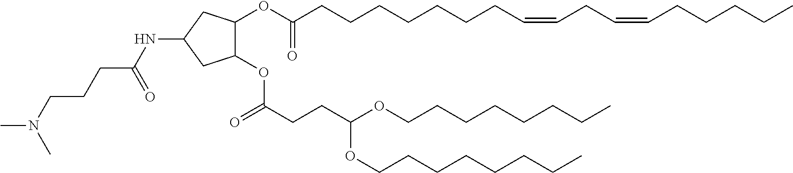 [SMILES: CCCCC/C=C\C/C=C\CCCCCCCC(=O)OC1CC(NC(=O)CCCN(C)C)CC1OC(=O)CCC(OCCCCCCCC)OCCCCCCCC] | CZ-8 |
| 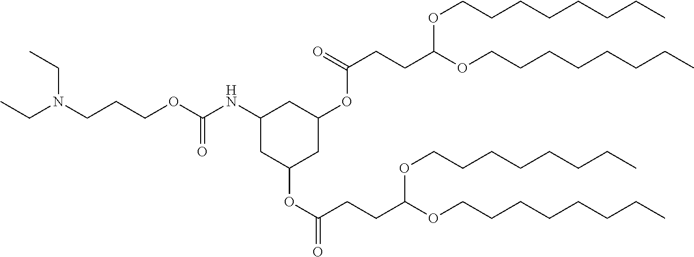 [SMILES: CCCCCCCCOC(CCC(=O)OC1CC(NC(=O)OCCCN(CC)CC)CC(OC(=O)CCC(OCCCCCCCC)OCCCCCCCC)C1)OCCCCCCCC] | CZ-9 |
| 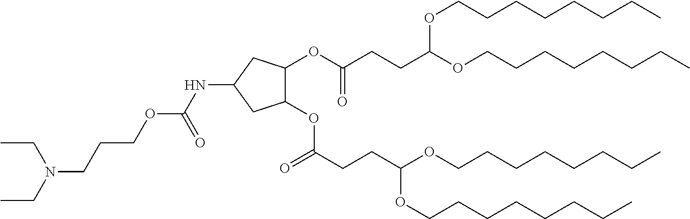 [SMILES: CCCCCCCCOC(CCC(=O)OC1CC(NC(=O)OCCCN(CC)CC)CC1OC(=O)CCC(OCCCCCCCC)OCCCCCCCC)OCCCCCCCC] | CZ-10 |
| 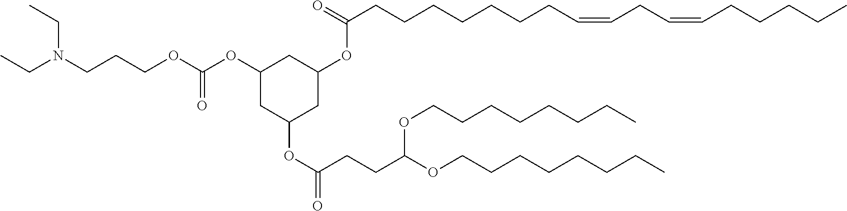 [SMILES: CCCCC/C=C\C/C=C\CCCCCCCC(=O)OC1CC(OC(=O)CCC(OCCCCCCCC)OCCCCCCCC)CC(OC(=O)OCCCN(CC)CC)C1] | CZ-11 |
| 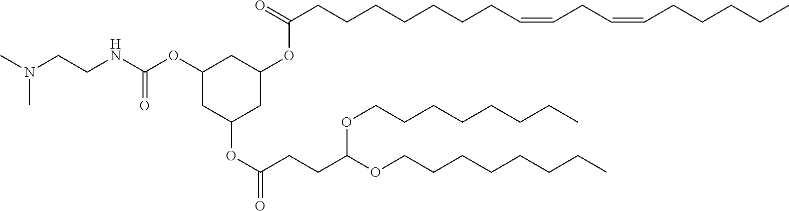 [SMILES: CCCCC/C=C\C/C=C\CCCCCCCC(=O)OC1CC(OC(=O)CCC(OCCCCCCCC)OCCCCCCCC)CC(OC(=O)NCCN(C)C)C1] | CZ-12 |
| 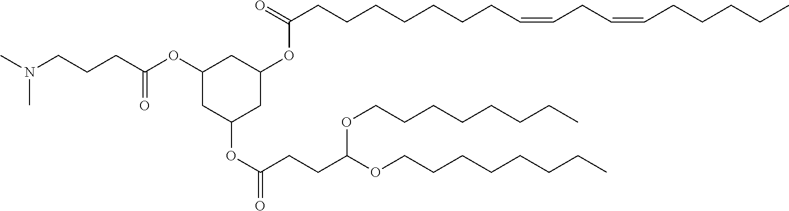 [SMILES: CCCCC/C=C\C/C=C\CCCCCCCC(=O)OC1CC(OC(=O)CCCN(C)C)CC(OC(=O)CCC(OCCCCCCCC)OCCCCCCCC)C1] | CZ-13 |
| 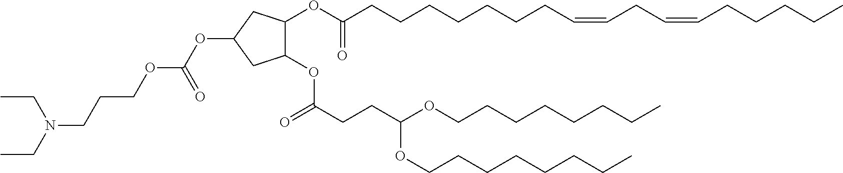 [SMILES: CCCCC/C=C\C/C=C\CCCCCCCC(=O)OC1CC(OC(=O)OCCCN(CC)CC)CC1OC(=O)CCC(OCCCCCCCC)OCCCCCCCC] | CZ-14 |
| 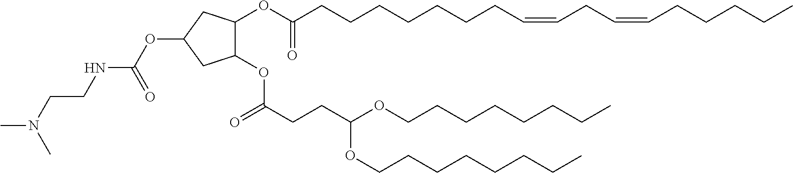 [SMILES: CCCCC/C=C\C/C=C\CCCCCCCC(=O)OC1CC(OC(=O)NCCN(C)C)CC1OC(=O)CCC(OCCCCCCCC)OCCCCCCCC] | CZ-15 |
| 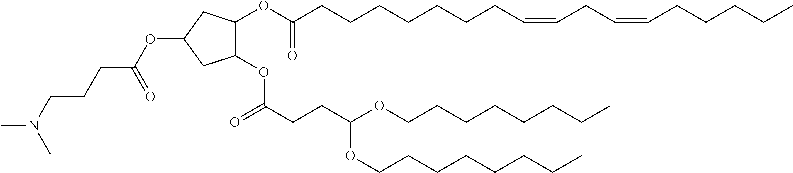 [SMILES: CCCCC/C=C\C/C=C\CCCCCCCC(=O)OC1CC(OC(=O)CCCN(C)C)CC1OC(=O)CCC(OCCCCCCCC)OCCCCCCCC] | CZ-16 |
| 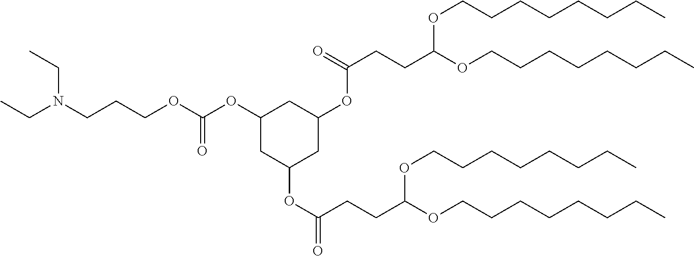 [SMILES: CCCCCCCCOC(CCC(=O)OC1CC(OC(=O)CCC(OCCCCCCCC)OCCCCCCCC)CC(OC(=O)OCCCN(CC)CC)C1)OCCCCCCCC] | CZ-17 |
| 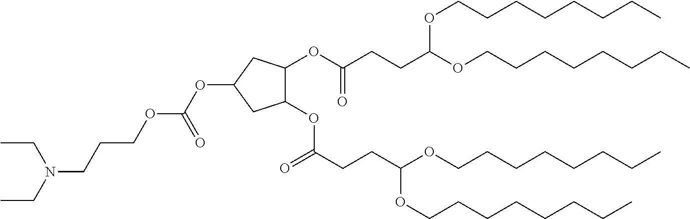 [SMILES: CCCCCCCCOC(CCC(=O)OC1CC(OC(=O)OCCCN(CC)CC)CC1OC(=O)CCC(OCCCCCCCC)OCCCCCCCC)OCCCCCCCC] | CZ-18 |

### Series “S”

Described below are a number of exemplary ionizable lipids of the present disclosure. In certain embodiments, the ionizable lipid is one selected from those disclosed in PCT Application PCT/US2024/019990, which is incorporated by reference herein, in its entirety.

### Formula (S-I)

In some embodiments, ionizable lipids of the present disclosure have a structure of Formula (S-I):

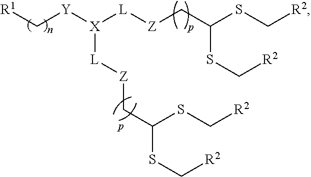

or a pharmaceutically acceptable salt thereof, wherein:
X is N or CH;
Y is a bond,

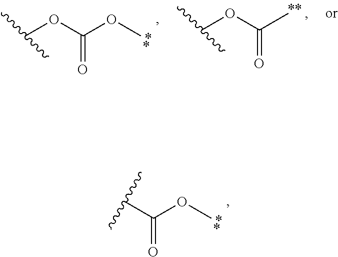

wherein bond marked with an “**” is attached to X; each Z is independently selected from the group consisting of:

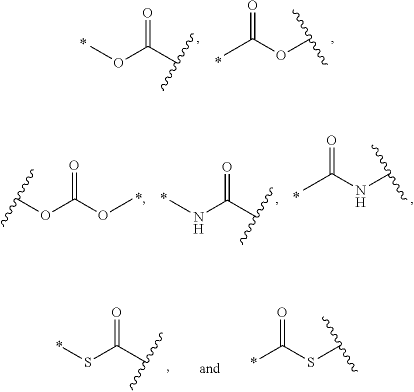

-S wherein the bond marked with an “*” is attached to L; each L is independently C₂-C₁₀ alkylenyl;
R¹ is OH, N(R³)₂,

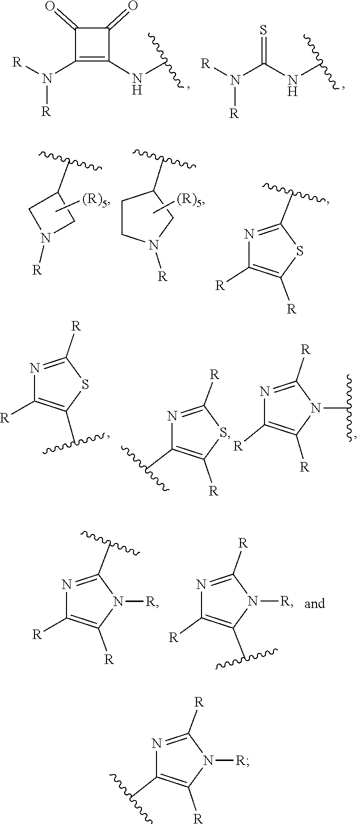

each R is independently —H or C₁-C₆ aliphatic;
each R² is independently selected from optionally substituted C₂₋₁₄alkyl and C₂₋₁₄alkenyl, wherein any —(CH₂)₂— of the C₂-C₁₄ alkyl can be optionally replaced with C₃-C₆ cycloalkylenyl;
each R³ independently selected from is H and C₁₋₆alkyl;
n is selected from 1 to 6; and each p is independently selected from 1 to 6.

### X

In some embodiments, ionizable lipids of the present disclosure have a structure of Formula (S-I), wherein X is N. In some embodiments, ionizable lipids of the present disclosure have a structure of Formula (S-I), wherein X is CH.

### Y

In some embodiments, ionizable lipids of the present disclosure have a structure of Formula (S-I), wherein Y is a bond. In some embodiments, ionizable lipids of the present disclosure have a structure of Formula (S-I), wherein Y is

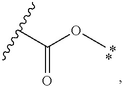 [SMILES: *C(=O)OC]

wherein bond marked with an “**” is attached to X. In some embodiments, ionizable lipids of the present disclosure have a structure of Formula (S-I), wherein Y is

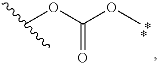 [SMILES: *OC(=O)OC]

wherein bond marked with an “**” is attached to X. In some embodiments, ionizable lipids of the present disclosure have a structure of Formula (S-I), wherein Y is

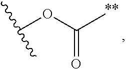 [SMILES: *OC(C)=O]

wherein bond marked with an “**” is attached to X.

### Z

In some embodiments, ionizable lipids of the present disclosure have a structure of Formula (S-I), wherein Z is

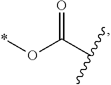 [SMILES: *OC(*)=O]

wherein bond marked with an “*” is attached to X. In some embodiments, ionizable lipids of the present disclosure have a structure of Formula (S-I), wherein Z is

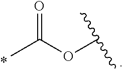 [SMILES: *OC(C)=O]

wherein bond marked with an “*” is attached to X. In some embodiments, ionizable lipids of the present disclosure have a structure of Formula (S-I), wherein Z is

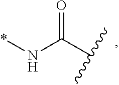 [SMILES: *NC(*)=O]

wherein bond marked with an “*” is attached to X. In some embodiments, ionizable lipids of the present disclosure have a structure of Formula (S-I), wherein Z is

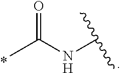

wherein bond marked with an “*” is attached to X. In some embodiments, ionizable lipids of the present disclosure have a structure of Formula (S-I), wherein Z is

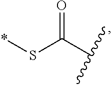 [SMILES: *SC(*)=O]

wherein bond marked with an “*” is attached to X. In some embodiments, ionizable lipids of the present disclosure have a structure of Formula (S-I), wherein Z is

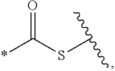 [SMILES: *SC(*)=O]

wherein bond marked with an “*” is attached to X. In some embodiments, ionizable lipids of the present disclosure have a structure of Formula (S-I), wherein Z is

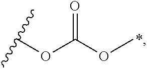

wherein bond marked with an “*” is attached to X.

### L

In some embodiments, ionizable lipids of the present disclosure have a structure of Formula (S-I), wherein L is C₂-C₁₀ alkylenyl. In some embodiments, ionizable lipids of the present disclosure have a structure of Formula (S-I), wherein L is C₅-C₈ alkylenyl. In some embodiments, ionizable lipids of the present disclosure have a structure of Formula (S-I), wherein L is C₅ alkylenyl. In some embodiments, ionizable lipids of the present disclosure have a structure of Formula (S-I), wherein L is C₆ alkylenyl. In some embodiments, ionizable lipids of the present disclosure have a structure of Formula (S-I), wherein L is C₇ alkylenyl. In some embodiments, ionizable lipids of the present disclosure have a structure of Formula (S-I), wherein L is C₈ alkylenyl.

### R¹

In some embodiments, ionizable lipids of the present disclosure have a structure of Formula (S-I), wherein R¹ is OH. In some embodiments, ionizable lipids of the present disclosure have a structure of Formula (S-I), wherein R¹ is N(R³)₂. In some embodiments, ionizable lipids of the present disclosure have a structure of Formula (S-I), wherein R¹ is

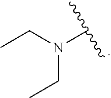 [SMILES: *N(CC)CC]

In some embodiments, ionizable lipids of the present disclosure have a structure of Formula (S-I), wherein R¹

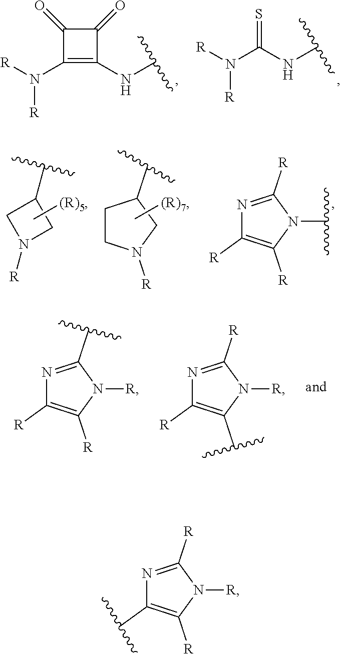

wherein each R is independently —H or C₁-C₆ aliphatic. In certain embodiments, R¹ is

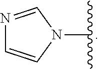 [SMILES: *n1ccnc1]

In some embodiments, ionizable lipids of the present disclosure have a structure of Formula (S-Ia):

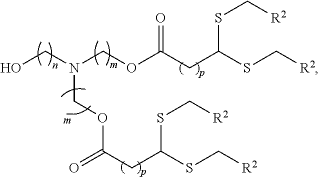

or a pharmaceutically acceptable salt thereof, wherein:
   each R² is independently selected from optionally substituted C₂₋₁₄alkyl and C₂₋₁₄ alkenyl, wherein any —(CH₂)₂— of the C₂-C₁₄ alkyl can be optionally replaced with C₃-C₆ cycloalkylenyl;
n is selected from 1 to 4;
each m is independently selected from 2 to 10; and
   each p is independently selected from 2 to 6.

In some embodiments, ionizable lipids of the present disclosure have a structure of Formula (S-Ib):

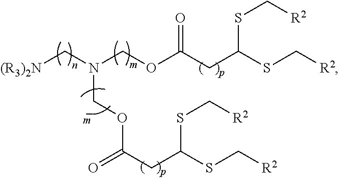

or a pharmaceutically acceptable salt thereof, wherein:
   each R² is independently selected from optionally substituted C₂₋₁₄alkyl and C₂₋₁₄ alkenyl, wherein any —(CH₂)₂— of the C₂-C₁₄ alkyl can be optionally replaced with C₃-C₆ cycloalkylenyl;
   each R³ independently selected from is H and C₁₋₆alkylene;
   n is selected from 1 to 4;
   each m is independently selected from 2 to 10; and
   each p is independently selected from 2 to 6.

### R²

In some embodiments, ionizable lipids of the present disclosure have a structure of Formula (S-I), Formula (S-Ia), or Formula (S-Ib), wherein R² is optionally substituted C₂₋₁₄alkyl. In some embodiments, ionizable lipids of the present disclosure have a structure of Formula (S-I), Formula (S-Ia), or Formula (S-Ib), wherein R² is optionally substituted C₇₋₁₂alkyl. In some embodiments, ionizable lipids of the present disclosure have a structure of Formula (S-I), Formula (S-Ia), or Formula (S-Ib), wherein R² is independently selected from the group consisting of:

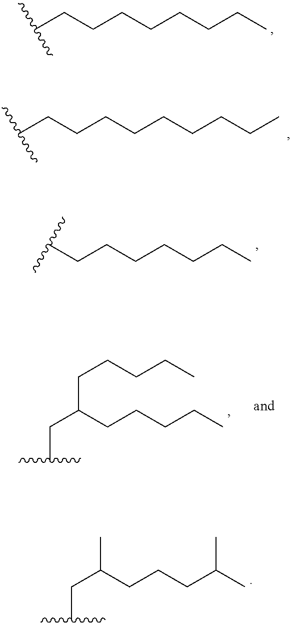

In some embodiments, ionizable lipids of the present disclosure have a structure of Formula (S-I), Formula (S-Ia), or Formula (S-Ib), wherein R² is

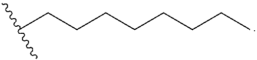 [SMILES: *CCCCCCCC]

In some embodiments, ionizable lipids of the present disclosure have a structure of Formula (S-I), Formula (S-Ia), or Formula (S-Ib), wherein R² is

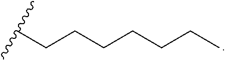 [SMILES: *CCCCCCC]

In some embodiments, ionizable lipids of the present disclosure have a structure of Formula (S-I), Formula (S-Ia), or Formula (S-Ib), wherein R² is

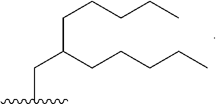 [SMILES: *CC(CCCCC)CCCCC ; "."]

In some embodiments, ionizable lipids of the present disclosure have a structure of Formula (S-I), Formula (S-Ia), or Formula (S-Ib), wherein R² is

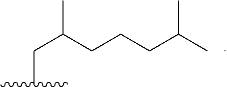 [SMILES: *CC(C)CCCC(C)C]

In some embodiments, ionizable lipids of the present disclosure have a structure of Formula (S-I), Formula (S-Ia), or Formula (S-Ib), wherein R² is optionally substituted C₂₋₁₄alkenyl. In some embodiments, ionizable lipids of the present disclosure have a structure of Formula (S-I), Formula (S-Ia), or Formula (S-Ib), wherein R² is independently selected from:

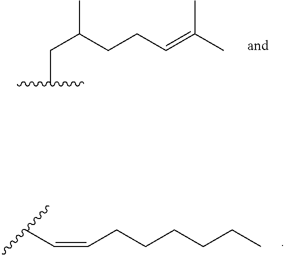

In some embodiments, ionizable lipids of the present disclosure have a structure of Formula (S-I), Formula (S-Ia), or Formula (S-Ib), wherein R² is

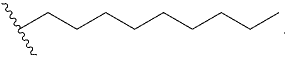 [SMILES: *CCCCCCCCC]

In some embodiments, ionizable lipids of the present disclosure have a structure of Formula (S-I), Formula (S-Ia), or Formula (S-Ib), wherein R² is optionally substituted C₈₋₉alkenyl. In some embodiments, ionizable lipids of the present disclosure have a structure of Formula (S-I), Formula (S-Ia), or Formula (S-Ib), wherein R² is

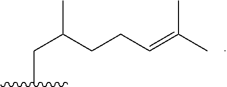 [SMILES: *CC(C)CCC=C(C)C]

In some embodiments, ionizable lipids of the present disclosure have a structure of Formula (S-I), Formula (S-Ia), or Formula (S-Ib), wherein R² is

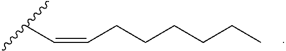 [SMILES: */C=C\CCCCCC]

In some embodiments, ionizable lipids of the present disclosure have a structure of Formula (S-I), Formula (S-Ia), or Formula (S-Ib), wherein R² is

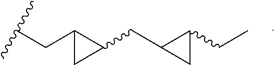 [SMILES: *CC1CC1CC1CC1CC]

### R³

In some embodiments, ionizable lipids of the present disclosure have a structure of Formula (S-I) or Formula (S-Ib), wherein R³ is hydrogen.

In some embodiments, ionizable lipids of the present disclosure have a structure of Formula (S-I), Formula (S-Ia), or Formula (S-Ib), wherein R³ is C₁₋₆alkylene. In some embodiments, ionizable lipids of the present disclosure have a structure of Formula (S-I), wherein each R³ is C₁alkyl, C₂alkyl, C₃alkyl, C₄alkyl, C₅alkyl, or C₆alkyl.

n

In some embodiments, ionizable lipids of the present disclosure have a structure of Formula (S-I), Formula (S-Ia), or Formula (S-Ib), wherein n is 3. In some embodiments, ionizable lipids of the present disclosure have a structure of Formula (S-I), Formula (S-Ia), or Formula (S-Ib), wherein n is 4. In some embodiments, ionizable lipids of the present disclosure have a structure of Formula (S-I), Formula (S-Ia), or Formula (S-Ib), wherein n is 1, 2, 5, or 6.

m

In some embodiments, ionizable lipids of the present disclosure have a structure of Formula (S-Ia), or Formula (S-Ib), wherein m is selected from 5 to 8. In some embodiments, ionizable lipids of the present disclosure have a structure of Formula (S-Ia), or Formula (S-Ib), wherein m is 5. In some embodiments, ionizable lipids of the present disclosure have a structure of Formula (S-Ia) or Formula (S-Ib), wherein m is 6. In some embodiments, ionizable lipids of the present disclosure have a structure of Formula (S-Ia) or Formula (S-Ib), wherein m is 7. In some embodiments, ionizable lipids of the present disclosure have a structure of Formula (S-Ia) or Formula (S-Ib), wherein m is 8. In some embodiments, ionizable lipids of the present disclosure have a structure of Formula (S-Ia), or Formula (S-Ib), wherein m is 2, 3, 4, 9, or 10.

### P

In some embodiments, ionizable lipids of the present disclosure have a structure of Formula (S-I), Formula (S-Ia), or Formula (S-Ib), wherein p is independently selected from 2 to 4. In some embodiments, ionizable lipids of the present disclosure have a structure of Formula (S-I), Formula (S-Ia), or Formula (S-Ib), wherein p is 2. In some embodiments, ionizable lipids of the present disclosure have a structure of Formula (S-I), Formula (S-Ia), or Formula (S-Ib), wherein p is 3. In some embodiments, ionizable lipids of the present disclosure have a structure of Formula (S-I), Formula (S-Ia), or Formula (S-Ib), wherein p is 4. In some embodiments, ionizable lipids of the present disclosure have a structure of Formula (S-I), Formula (S-Ia), or Formula (S-Ib), wherein p is 5 or 6. In some embodiments, ionizable lipids of the present disclosure have a structure of Formula (S-I), wherein p is 1.

### Formula (S-M)

In some embodiments, ionizable lipids of the present disclosure have a structure of Formula (S-M):

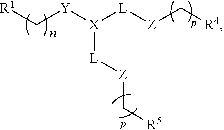

or a pharmaceutically acceptable salt thereof, wherein:
X is N or CH;
Y is a bond,

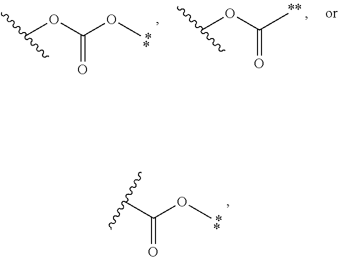

wherein bond marked with an “**” is attached to X;
each Z is independently selected from the group consisting of:

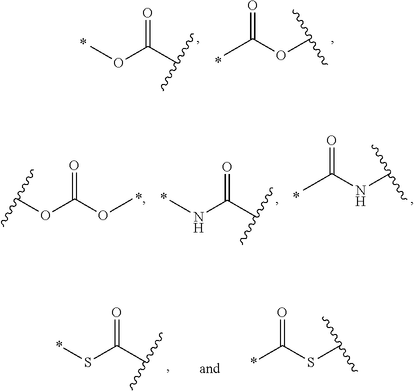

wherein the bond marked with an “*” is attached to L;
   each L is independently C₂-C₁₀ alkylenyl;
   R¹ is OH, N(R³)₂,

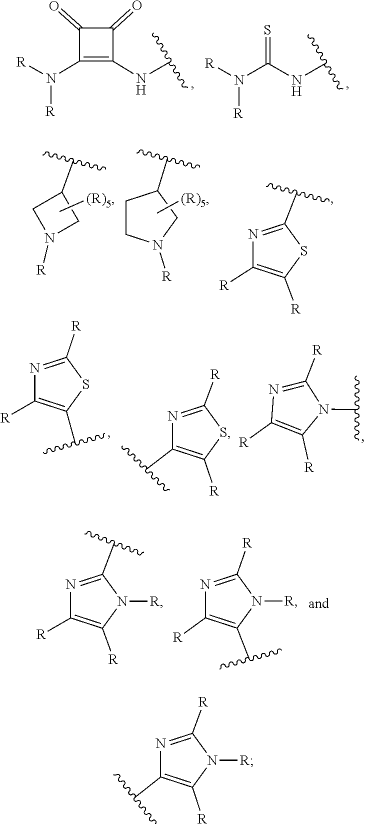

each R is independently —H or C₁-C₆ aliphatic;
   each R³ independently selected from is H and C₁₋₆alkyl;
R⁴ is —CH(SR⁶)(SR⁷);
   R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
   R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂cycloalkylenyl, phenyl, —O—, —NH—, —S—, —SS—, —C(O)—, —OC(O)O—, —OC(O)—, —NHC(O)— or —C(O)O—; and
   R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂cycloalkylenyl, phenyl, —O—, —NH—, —S—, —SS—, —C(O)—, —OC(O)O—, —OC(O)—, —NHC(O)— or —C(O)O—;
      n is selected from 1 to 6; and
      each p is independently selected from 1 to 6.

In some embodiments, ionizable lipids of the present disclosure have a structure of Formula (S-Ma)

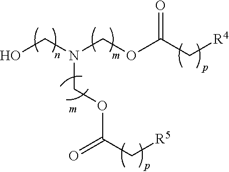

or a pharmaceutically acceptable salt thereof, wherein:
   n is selected from 1 to 4;
   each m is independently selected from 2 to 10; and
   each p is independently selected from 2 to 6.

In some embodiments, ionizable lipids of the present disclosure have a structure of Formula (S-Mb)

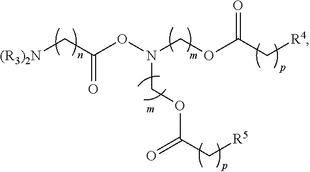

or a pharmaceutically acceptable salt thereof, wherein:
   each R³ independently selected from is H and C₁₋₆ alkyl;
   n is selected from 1 to 4;
   each m is independently selected from 2 to 10; and
each p is independently selected from 2 to 6.

### R¹

In some embodiments, ionizable lipids of the present disclosure have a structure of Formula (S-M), wherein R¹ is OH. In some embodiments, ionizable lipids of the present disclosure have a structure of Formula (S-I), wherein R¹ is N(R³)₂. In some embodiments, ionizable lipids of the present disclosure have a structure of Formula (S-M), wherein R¹ is

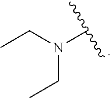 [SMILES: *N(CC)CC]

In some embodiments, ionizable lipids of the present disclosure have a structure of Formula (S-M), wherein R¹ is

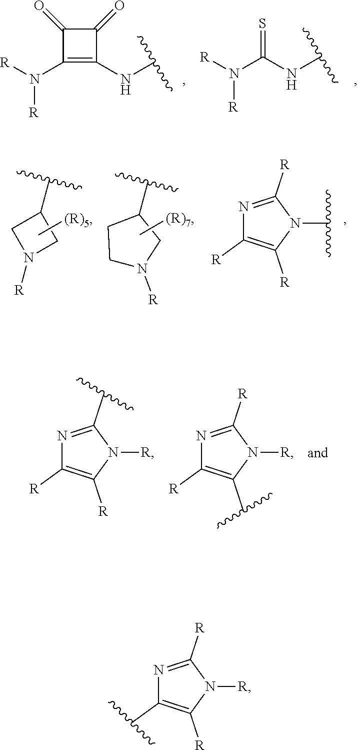

wherein each R is independently —H or C₁-C₆ aliphatic. In certain embodiments, R¹ is

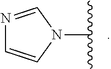 [SMILES: *n1ccnc1]

n

In some embodiments, ionizable lipids of the present disclosure have a structure of Formula (S-M), Formula (S-Ma), or Formula (S-Mb), wherein n is 3. In some embodiments, ionizable lipids of the present disclosure have a structure of Formula (S-M), Formula (S-Ma), or Formula (S-Mb), wherein n is 4. In some embodiments, ionizable lipids of the present disclosure have a structure of Formula (S-M), Formula (S-Ma), or Formula (S-Mb), wherein n is 1, 2, 5, or 6.

p

In some embodiments, ionizable lipids of the present disclosure have a structure of Formula (S-M), Formula (S-Ma), or Formula (S-Mb), wherein p is independently selected from 2 to 4. In some embodiments, ionizable lipids of the present disclosure have a structure of Formula (S-M), Formula (S-Ma), or Formula (S-Mb), wherein p is 2. In some embodiments, ionizable lipids of the present disclosure have a structure of Formula (S-M), Formula (S-Ma), or Formula (S-Mb), wherein p is 3. In some embodiments, ionizable lipids of the present disclosure have a structure of Formula (S-M), Formula (S-Ma), or Formula (S-Mb), wherein p is 4. In some embodiments, ionizable lipids of the present disclosure have a structure of Formula (S-M), Formula (S-Ma), or Formula (S-Mb), wherein p is 5 or 6. In some embodiments, ionizable lipids of the present disclosure have a structure of Formula (S-M), wherein p is 1.

### R⁵

As disclosed in Formula (S-M), in certain embodiments, R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹). In certain embodiments, R⁴ and R⁵ are the same. In certain embodiments, R⁴ and R⁵ are different.

In certain embodiments, R⁵ is selected from

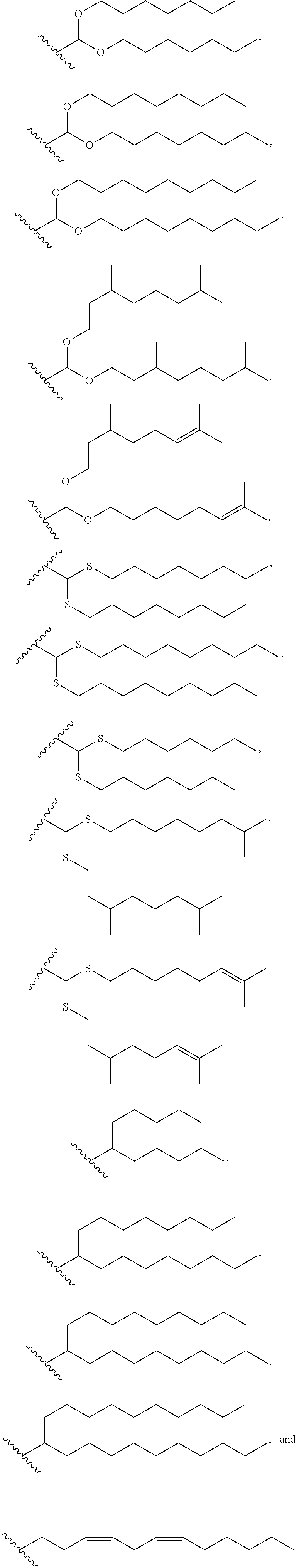

R⁶ and R⁷

As disclosed in Formula (S-M), in certain embodiments, R⁶ and R⁷ are each independently optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkylene. In certain embodiments, R⁶ is optionally substituted C₁₋₁₄ branched alkylene. In certain embodiments, R⁶ is optionally substituted C₁₋₁₄ straight chain alkylene. In certain embodiments, R⁶ is optionally substituted C₁-C₁₄ alkenylene. In certain embodiments, R⁶ is optionally substituted C₁₋₁₄ branched alkenylene. In certain embodiments, R⁶ is optionally substituted C₁₋₁₄ straight chain alkenylene. In certain embodiments, R⁶ is optionally substituted C₆-C₁₀ alkylene. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₅ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

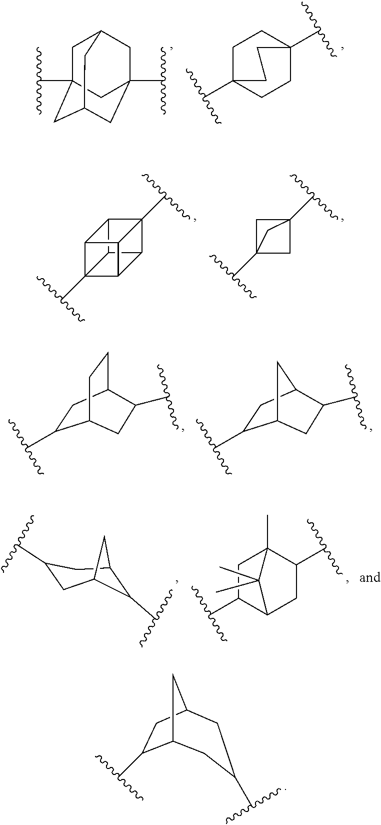

In certain embodiments, R⁷ is optionally substituted C₁-C₁₄ aliphatic. In certain embodiments, R⁷ is optionally substituted C₁-C₁₄ alkylene. In certain embodiments, R⁷ is optionally substituted C₁₋₁₄ branched alkylene. In certain embodiments, R⁷ is optionally substituted C₁-C₁₄ straight chain alkylene. In certain embodiments, R⁷ is optionally substituted C₁-C₁₄ alkenylene. In certain embodiments, R⁷ is optionally substituted C₁₋₁₄ branched alkenylene. In certain embodiments, R⁷ is optionally substituted C₁₋₁₄ straight chain alkenylene. In certain embodiments, R⁷ is optionally substituted C₆-C₁₀ alkylene. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

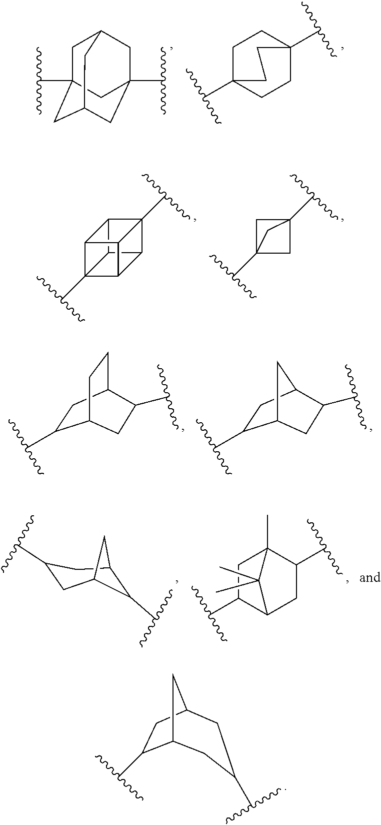

In certain embodiments, R⁶ and R⁷ are selected from

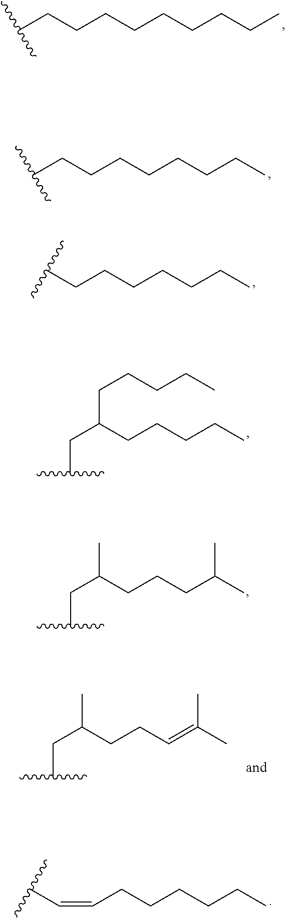

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

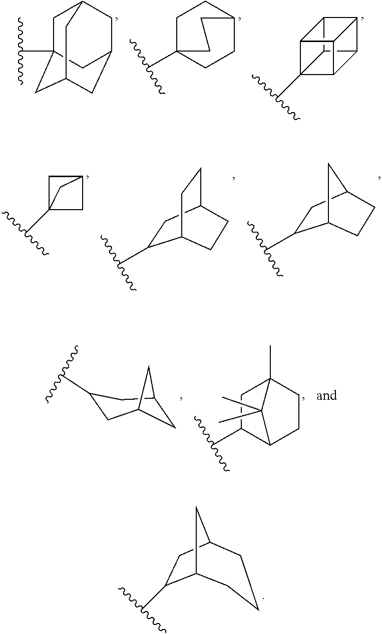

In certain embodiments, the substituted bridged bicyclic
or multicyclic C₅-C₁₂ cycloalkylenyl is a structure selected from

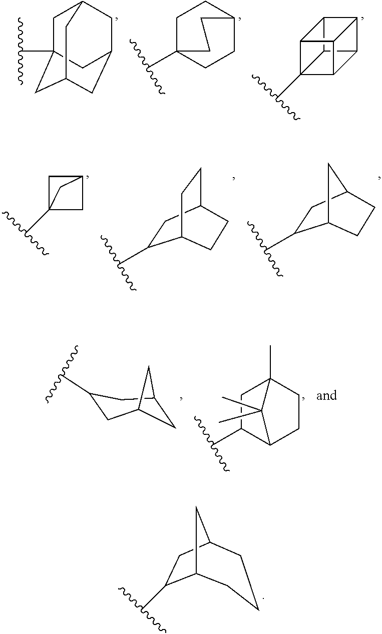

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from

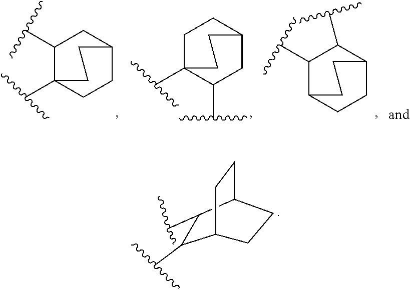

R⁸ and R⁹

As disclosed in Formula (S-M), in certain embodiments, R⁸ and R⁹ are each independently optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R8 and R9 are the same. In certain embodiments, R8 and R9 are different.

In certain embodiments, R8 is optionally substituted C1-C14 aliphatic. In certain embodiments, R8 is optionally substituted C1-C14 alkylene. In certain embodiments, R8 is optionally substituted C1-C14 branched alkylene. In certain embodiments, R8 is optionally substituted C1-C14 straight chain alkylene. In certain embodiments, R8 is optionally substituted C1-C14 alkenylene. In certain embodiments, R8 is optionally substituted C1-C14 branched alkenylene. In certain embodiments, R8 is optionally substituted C1-C14 straight chain alkenylene. In certain embodiments, R8 is optionally substituted C6-C10 alkylene. In certain embodiments, R8 is optionally substituted —(CH2)5CH3. In certain embodiments, R8 is optionally substituted —(CH2)6CH3. In certain embodiments, R8 is optionally substituted —(CH2)7CH3. In certain embodiments, R8 is optionally substituted —(CH2)8CH3. In certain embodiments, R8 is optionally substituted —(CH2)9CH3.

In certain embodiments, one of the methylene linkages of R8 is replaced with an optionally substituted C3-C8 cycloalkylenyl, an optionally substituted bridged bicyclic or multicyclic C5-C12 cycloalkylenyl. In certain embodiments, the optionally substituted bridged bicyclic or multicyclic C5-C12 cycloalkylenyl is selected from:

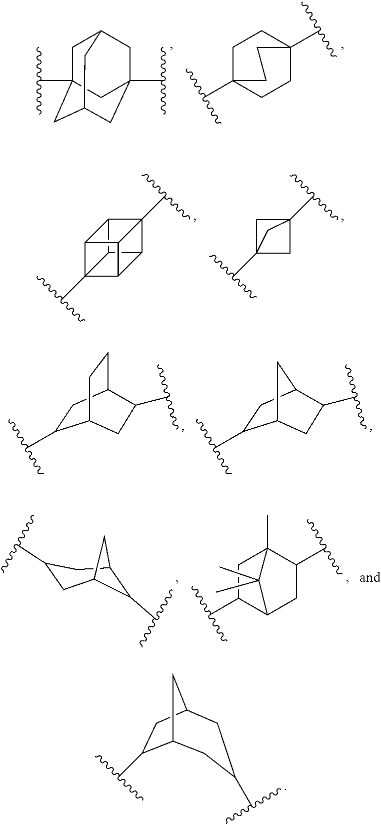

In certain embodiments, R⁹ is optionally substituted C₁-C₁₄ aliphatic. In certain embodiments, R⁹ is optionally substituted C₁-C₁₄ alkylene. In certain embodiments, R⁹ is optionally substituted C₁-C₁₄ branched alkylene. In certain embodiments, R9 is optionally substituted C1-C14 straight chain alkylene. In certain embodiments, R9 is optionally substituted C1-C14 alkenylene. In certain embodiments, R9 is optionally substituted C1-C14 branched alkenylene. In certain embodiments, R9 is optionally substituted C1-C14 straight chain alkenylene. In certain embodiments, R9 is optionally substituted C6-C10 alkylene. In certain embodiments, R9 is optionally substituted —(CH2)5CH3. In certain embodiments, R9 is optionally substituted —(CH2)6CH3. In certain embodiments, R9 is optionally substituted —(CH2)7CH3. In certain embodiments, R9 is optionally substituted —(CH2)8CH3. In certain embodiments, R9 is optionally substituted —(CH2)9CH3.

In certain embodiments, one of the methylene linkages of R⁹ is replaced with an optionally substituted C₃-C₅ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

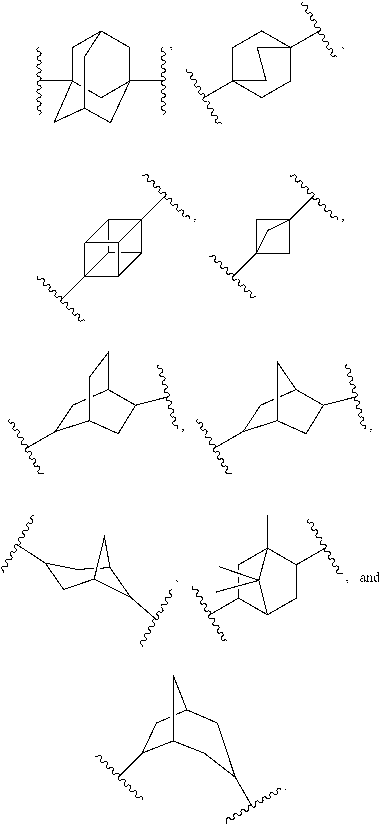

In certain embodiments, R8 and R9 are selected from

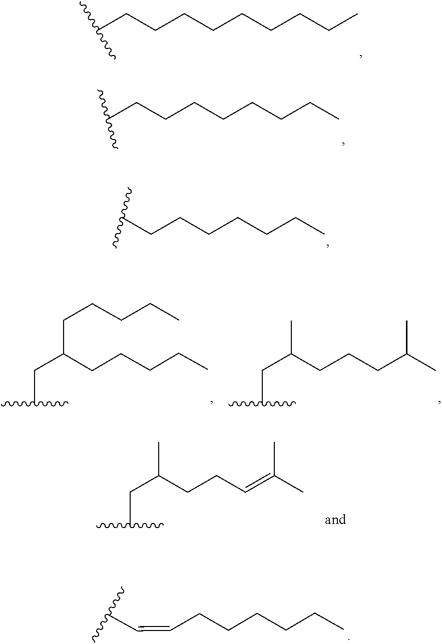

In some embodiments, R8 and R9 taken together form an optionally substituted bridged bicyclic or multicyclic C4-C14 cycloalkyl or optionally substituted bridged bicyclic or multicyclic 4-14 membered heterocyclyl.

In certain embodiments, each R8 and R9 are each independently selected from an optionally substituted bridged bicyclic C5-C12 cycloalkylenyl. In certain embodiments, R8 is an optionally substituted bridged multicyclic C5-C12 cycloalkylenyl. In certain embodiments, R⁹ is an optionally substituted bridged bicyclic C5-C12 cycloalkylenyl. In certain embodiments, the optionally substituted bridged bicyclic or multicyclic C5-C12 cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

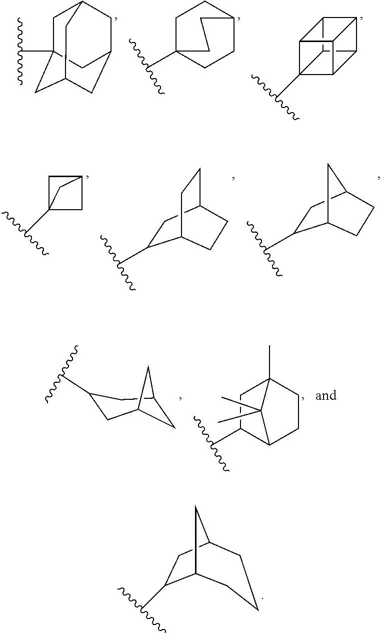

In certain embodiments the substituted bridged bicyclic or multicyclic C₅-C₁₂ cycloalkylenyl is a structure selected from

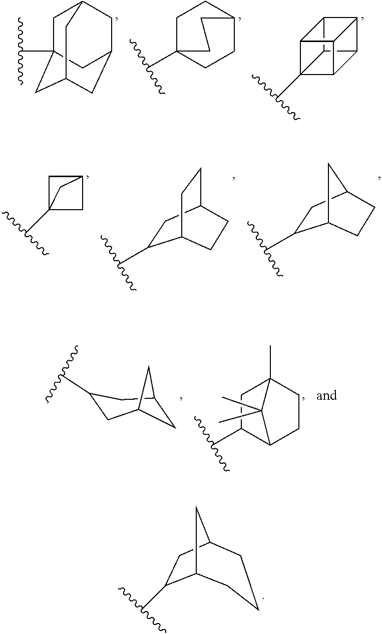

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

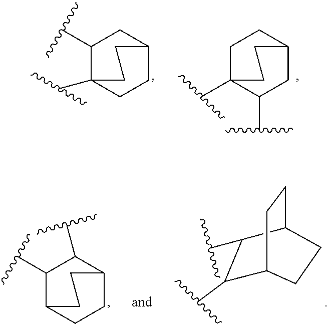 [SMILES: *C1CC2CCC1(*)CC2 ; *C1CC2CCC1(*)CC2 ; *C1C2CCC(CC2)C1* ; *C1C2CCC(CC2)C1*]

In some embodiments, ionizable lipids of the present disclosure comprise an acyclic core. In some embodiments, ionizable lipids of the present disclosure are selected from any lipid in Table (I-F) below or a pharmaceutically acceptable salt thereof:

TABLE (I-F)
Non-Limiting Examples of Ionizable Lipids

| Cmpd | Structure |
|---|---|
| S-1 | 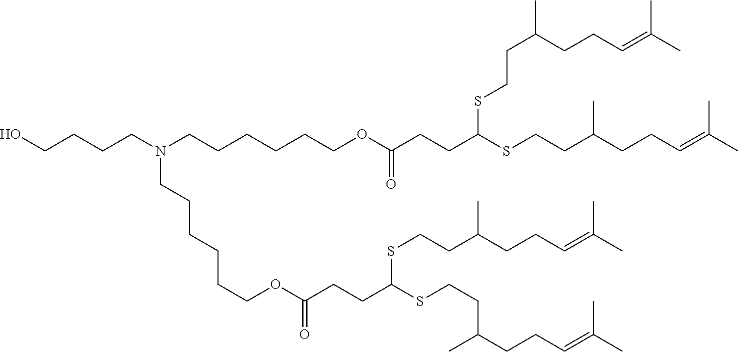 [SMILES: CC(C)=CCCC(C)CCSC(CCC(=O)OCCCCCCN(CCCCO)CCCCCCOC(=O)CCC(SCCC(C)CCC=C(C)C)SCCC(C)CCC=C(C)C)SCCC(C)CCC=C(C)C] |
| S-2 | 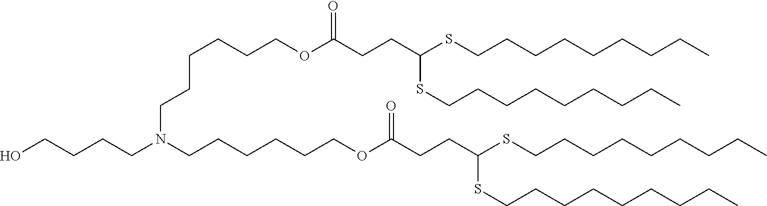 [SMILES: CCCCCCCCCSC(CCC(=O)OCCCCCCN(CCCCO)CCCCCCOC(=O)CCC(SCCCCCCCCC)SCCCCCCCCC)SCCCCCCCCC] |
| S-3 | 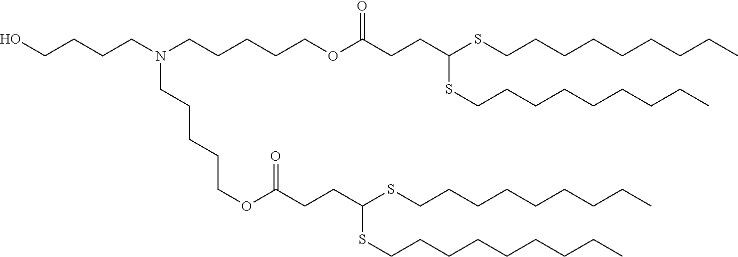 [SMILES: CCCCCCCCCSC(CCC(=O)OCCCCCN(CCCCO)CCCCCOC(=O)CCC(SCCCCCCCCC)SCCCCCCCCC)SCCCCCCCCC] |
| S-4 | 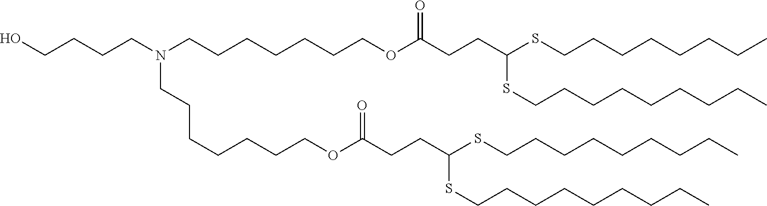 [SMILES: CCCCCCCCCSC(CCC(=O)OCCCCCCCN(CCCCO)CCCCCCCOC(=O)CCC(SCCCCCCCCC)SCCCCCCCCC)SCCCCCCCC] |
| S-5 | 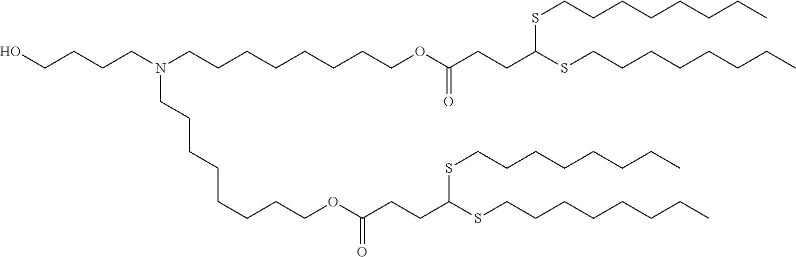 [SMILES: CCCCCCCCSC(CCC(=O)OCCCCCCCCN(CCCCO)CCCCCCCCOC(=O)CCC(SCCCCCCCC)SCCCCCCCC)SCCCCCCCC] |
| S-6 | 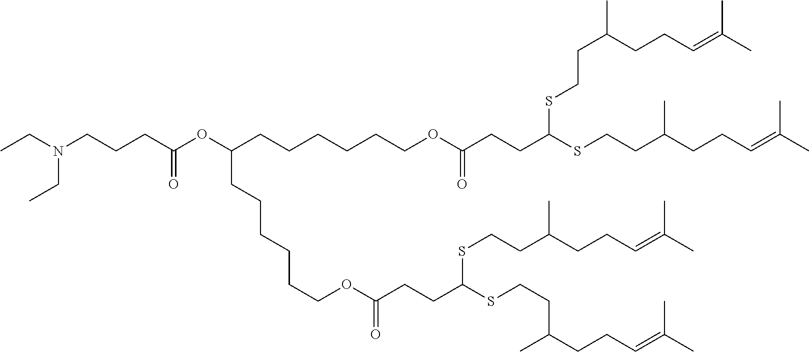 [SMILES: CCN(CC)CCCC(=O)OC(CCCCCCOC(=O)CCC(SCCC(C)CCC=C(C)C)SCCC(C)CCC=C(C)C)CCCCCCOC(=O)CCC(SCCC(C)CCC=C(C)C)SCCC(C)CCC=C(C)C] |
| S-7 | 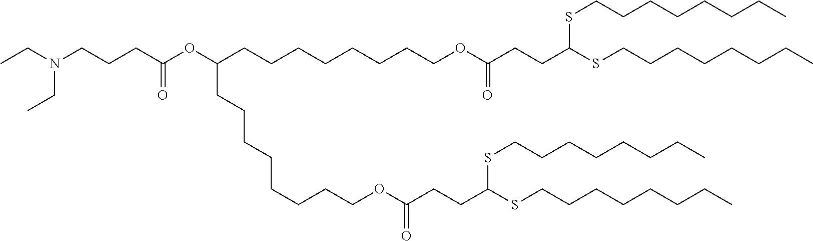 [SMILES: CCCCCCCCSC(CCC(=O)OCCCCCCCCC(CCCCCCCCOC(=O)CCC(SCCCCCCCC)SCCCCCCCC)OC(=O)CCCN(CC)CC)SCCCCCCCC] |
| S-8 | 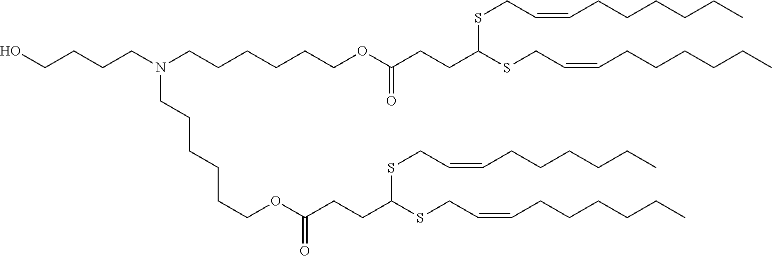 [SMILES: CCCCCC/C=C\CSC(CCC(=O)OCCCCCCN(CCCCO)CCCCCCOC(=O)CCC(SC/C=C\CCCCCC)SC/C=C\CCCCCC)SC/C=C\CCCCCC] |
| S-9 | 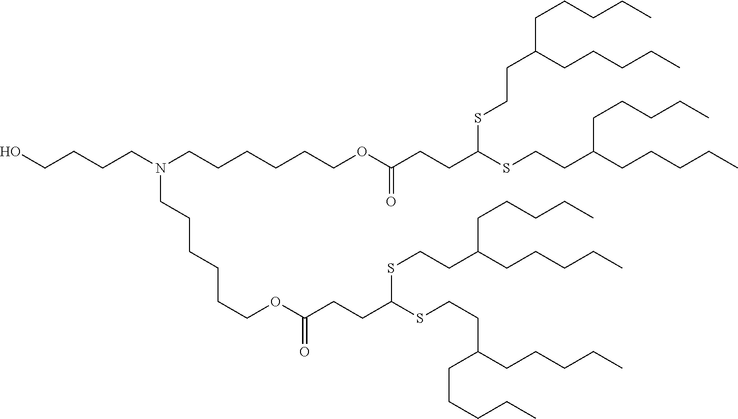 [SMILES: CCCCCC(CCCCC)CCSC(CCC(=O)OCCCCCCN(CCCCO)CCCCCCOC(=O)CCC(SCCC(CCCCC)CCCCC)SCCC(CCCCC)CCCCC)SCCC(CCCCC)CCCCC] |
| S-10 | 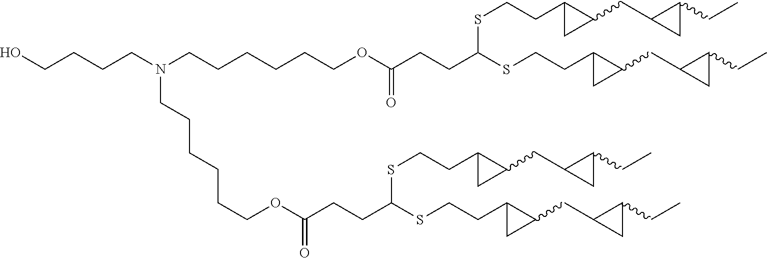 [SMILES: CCC1CC1CC1CC1CCSC(CCC(=O)OCCCCCCN(CCCCO)CCCCCCOC(=O)CCC(SCCC1CC1CC1CC1CC)SCCC1CC1CC1CC1CC)SCCC1CC1CC1CC1CC] |
| S-11 | 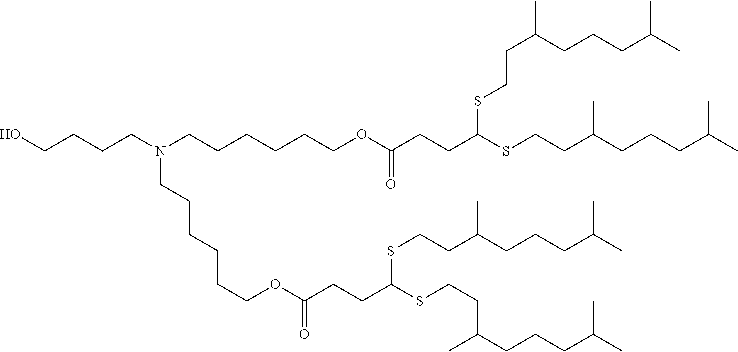 [SMILES: CC(C)CCCC(C)CCSC(CCC(=O)OCCCCCCN(CCCCO)CCCCCCOC(=O)CCC(SCCC(C)CCCC(C)C)SCCC(C)CCCC(C)C)SCCC(C)CCCC(C)C] |

Described below are a number of exemplary ionizable lipids of the present disclosure. In certain embodiments, the ionizable lipid is one selected from those disclosed in PCT Application PCT/US2024/019990, which is incorporated by reference herein, in its entirety.

In some embodiments, ionizable lipids of the present disclosure have a structure of Formula (AT)

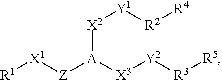

or a pharmaceutically acceptable salt thereof, wherein:
i) A is N; Z is a bond; X¹ is optionally substituted C₁-C₆ aliphatic, wherein the optional substituent is not oxo when X¹ is C₁ aliphatic; and R¹ is selected from the group consisting of:

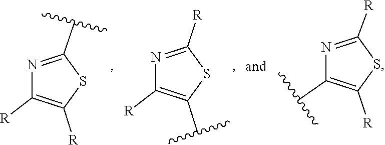

or
ii) A is CH;
   Z is

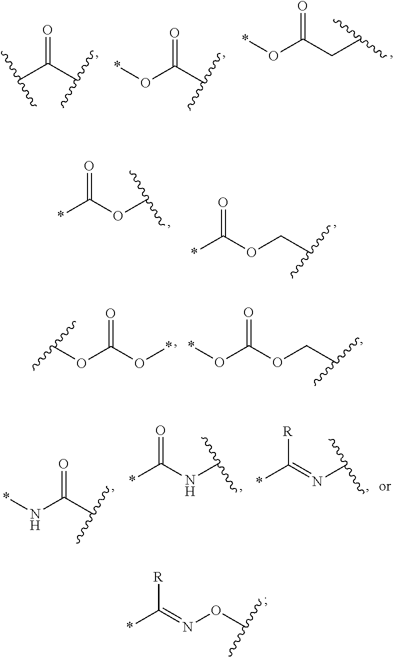

wherein the bond marked with an “*” is attached to X¹;
X¹ is a bond or optionally substituted C₁-C₆ aliphatic;
R¹ is selected from the group consisting of:

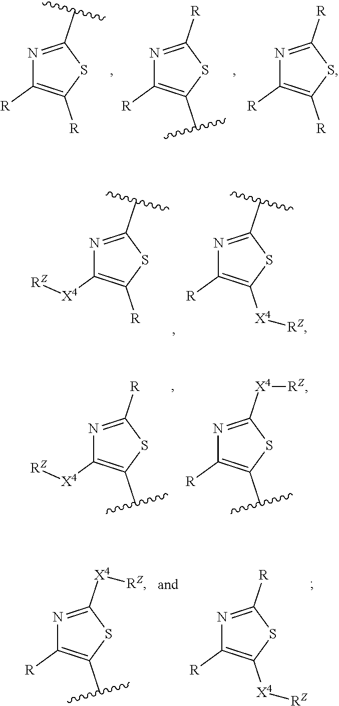

X⁴ is a bond or optionally substituted C₁-C₆ aliphatic;
R^{Z} is NR₂ or OH;
each R is independently —H or C₁-C₆ aliphatic;
X² and X³ are each independently optionally substituted C₁-C₁₂ aliphatic;
Y¹ and Y² are independently selected from the group consisting of

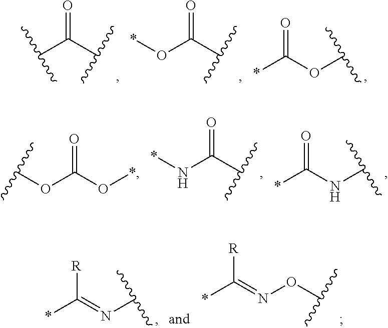

wherein the bond marked with an “*” is attached to X² for Y¹ or X³ for Y²;
R² is optionally substituted C₁-C₆ aliphatic;
R³ is optionally substituted C₁-C₆ aliphatic;
R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹), —CH(SR⁸)(SR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

### Formula (AT-E′)

In certain embodiments, ionizable lipids of the present disclosure have a structure of Formula (AT), wherein the ionizable lipids of the present disclosure have a structure of Formula (AT-E′):

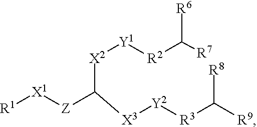

or a pharmaceutically acceptable salt thereof, wherein R¹, R, X¹, Z, X², X³, X⁴, R^{Z}, Y¹, Y², R², R³, R⁶, R⁷, R⁸, and R⁹ are as described in Formula (AT) or as otherwise described in any embodiments below.

### Formula (AT-F″′)

In certain embodiments, ionizable lipids of the present disclosure have a structure of Formula (AT), wherein the ionizable lipids of the present disclosure have a structure of Formula (AT-F″′):

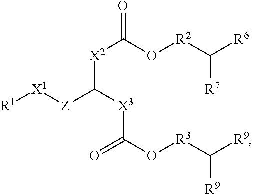

or a pharmaceutically acceptable salt thereof, wherein R¹, R, X¹, Z, X², X³, X⁴, R^{Z}, R², R³, R⁶, R⁷, R⁸, and R⁹ are as described in Formula (AT) or as otherwise described in any embodiments below.

### Formula (AT-M)

In certain embodiments, ionizable lipids of the present disclosure have a structure of Formula (AT), wherein the ionizable lipids of the present disclosure have a structure of Formula (AT-M):

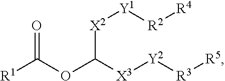

or a pharmaceutically acceptable salt thereof, wherein R¹, R, X², X³, X⁴, R^{Z}, Y¹, Y², R², R³, R⁴, R⁵, R⁶, R⁷, R⁸, and R⁹ are as described in Formula (AT) or as otherwise described in any embodiments below.

### Formula (AT-N′)

In certain embodiments, ionizable lipids of the present disclosure have a structure of Formula (AT), wherein the ionizable lipids of the present disclosure have a structure of Formula (AT-N′):

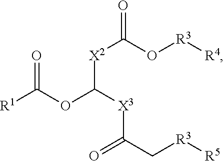

or a pharmaceutically acceptable salt thereof, wherein R¹, R, X¹, X², X³, X⁴, R^{Z}, R², R³, R⁴, R⁵, R⁶, R⁷, R⁸, and R⁹ are as described in Formula (AT) or as otherwise described in any embodiments below.

### Formula (AT-O′)

In certain embodiments, ionizable lipids of the present disclosure have a structure of Formula (AT), wherein the ionizable lipids of the present disclosure have a structure of Formula (AT-O′):

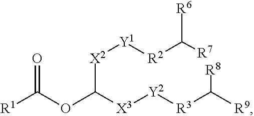

or a pharmaceutically acceptable salt thereof, wherein R¹, R, X¹, X², X³, Y¹, Y², X⁴, R^{Z}, R², R³, R⁶, R⁷, R⁸, and R⁹ are as described in Formula (AT) or as otherwise described in any embodiments below.

### Formula (AT-P″′)

In certain embodiments, ionizable lipids of the present disclosure have a structure of Formula (AT), wherein the ionizable lipids of the present disclosure have a structure of Formula (AT-P″′):

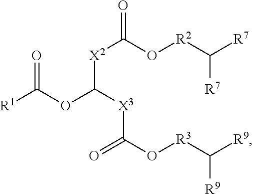

or a pharmaceutically acceptable salt thereof, wherein R¹, R, X¹, X², X³, R², R³, X⁴, R⁵, R⁶, R⁷, R⁸, and R⁹ are as described in Formula (AT) or as otherwise described in any embodiments below.

### A

As disclosed in Formula (AT), in certain embodiments, A is CH or N. In certain embodiments, A is CH. In certain embodiments, A is N.

### Z

As disclosed in Formula (AT), in certain embodiments wherein A is CH, Z is,

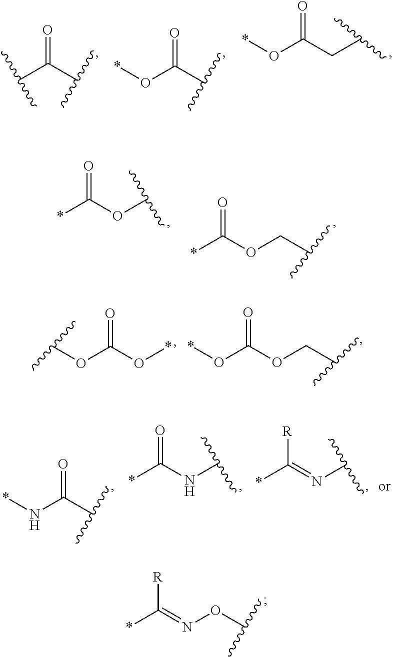

wherein the bond marked with an “*” is attached to X¹. In certain embodiments wherein A is CH, Z is

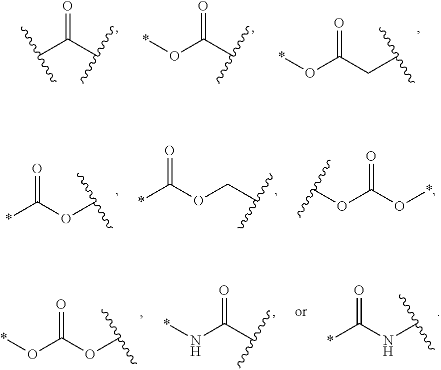

In certain embodiments wherein A is CH, Z is

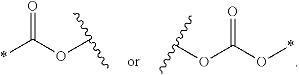 [SMILES: *OC(*)=O ; *OC(=O)O*]

In certain embodiments, Z is

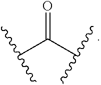 [SMILES: *C(*)=O]

In certain embodiments, Z is

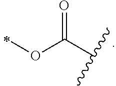

In certain embodiments, Z is

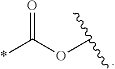 [SMILES: *OC(*)=O]

In certain embodiments, Z

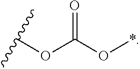

In certain embodiments, Z is

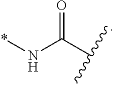 [SMILES: *NC(*)=O]

In certain embodiments, Z is

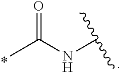

In certain embodiments, Z is

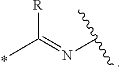

In certain embodiments, Z is

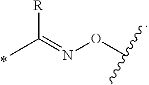

In certain embodiments, Z is

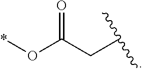 [SMILES: *CC(=O)O*]

In certain embodiments, Z is

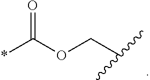 [SMILES: *COC(*)=O]

In certain embodiments, Z is

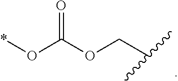 [SMILES: *COC(=O)O*]

As disclosed in Formula (AT), in certain embodiments wherein A is N, Z is a bond.

### X¹

As disclosed in Formula (AT), in certain embodiments wherein A is N, X¹ is optionally substituted C₁-C₆ aliphatic. In certain embodiments wherein A is N, X¹ is unsubstituted C₁-C₆ aliphatic. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

As disclosed in Formula (AT), in certain embodiments wherein A is CH, X¹ is a bond or optionally substituted C₁-C₆ aliphatic. In certain embodiments, X¹ is a bond. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### R¹

As disclosed in Formula (AT), in certain embodiments wherein A is N, R¹ is selected from the group consisting of

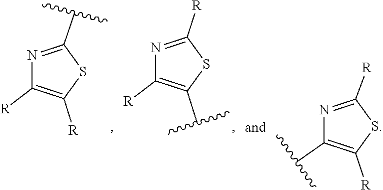

As disclosed in Formula (AT), in certain embodiments wherein A is CH, R¹ is selected from the group consisting of

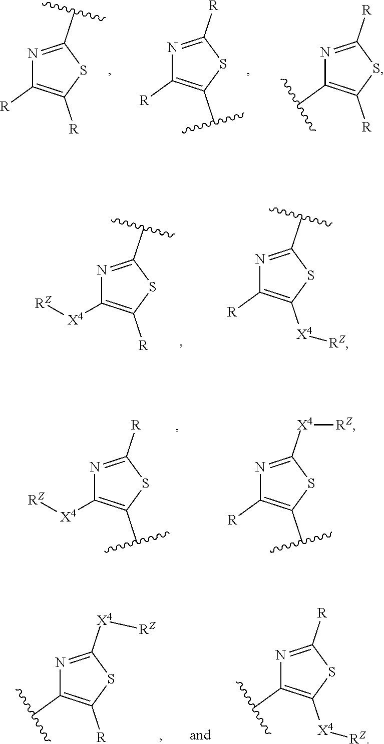

In certain embodiments, R¹ is

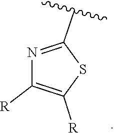

In certain embodiments, R¹ is

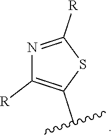

In certain embodiments, R¹ is

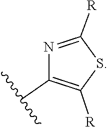

In certain embodiments, R¹ is

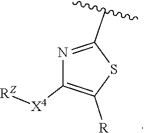

In certain embodiments, R¹ is

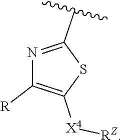

In certain embodiments, R¹ is

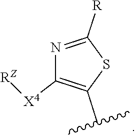

In certain embodiments, R¹ is

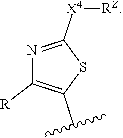

In certain embodiments, R¹ is

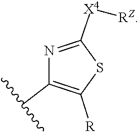

In certain embodiments, R¹ is

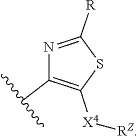

In certain embodiments, R¹ is

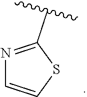 [SMILES: *c1nccs1]

In certain embodiments, R¹ is

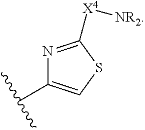

In certain embodiments, R¹ is.

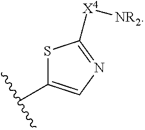

In certain embodiments, R¹ is

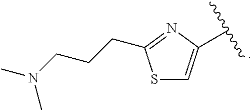 [SMILES: *c1csc(CCCN(C)C)n1]

X² and X³

As disclosed in Formula (AT), in certain embodiments, X2 and X3 are each independently optionally substituted C1-C12 aliphatic. In certain embodiments, X2 and X3 are the same. In certain embodiments, X2 and X3 are different.

In certain embodiments, X2 is an optionally substituted C1-C12 alkylene. In certain embodiments, X2 is an optionally substituted C1-C12 alkenylene. In certain embodiments, X2 is an optionally substituted C1-C10 aliphatic. In certain embodiments, X2 is an optionally substituted C1-C10 alkylene. In certain embodiments, X2 is an optionally substituted C1-C10 alkenylene. In certain embodiments, X2 is an optionally substituted C1-C8 aliphatic. In certain embodiments, X2 is an optionally substituted C1-C8 alkylene. In certain embodiments, X2 is an optionally substituted C1-C8 alkenylene. In certain embodiments, X2 is an optionally substituted C1-C6 aliphatic. In certain embodiments, X2 is an optionally substituted C1-C6 alkylene. In certain embodiments, X2 is an optionally substituted C1-C6 alkenylene. In certain embodiments, X2 is an optionally substituted C2-C12 aliphatic. In certain embodiments, X2 is an optionally substituted C2-C12 alkylene. In certain embodiments, X2 is an optionally substituted C2-C12 alkenylene. In certain embodiments, X2 is an optionally substituted C4-C12 aliphatic. In certain embodiments, X2 is an optionally substituted C4-C12 alkylene. In certain embodiments, X2 is an optionally substituted C4-C12 alkenylene. In certain embodiments, X2 is an optionally substituted C4-C10 aliphatic. In certain embodiments, X2 is an optionally substituted C4-C10 alkylene. In certain embodiments, X2 is an optionally substituted C4-C10 alkenylene. In certain embodiments, X2 is an optionally substituted C6-C8 aliphatic. In certain embodiments, X2 is an optionally substituted C6-C8 alkylene. In certain embodiments, X2 is an optionally substituted C6-C8 alkenylene. In certain embodiments, X2 is —(CH2)-. In certain embodiments, X2 is —(CH2)2-. In certain embodiments, X2 is —(CH2)3-. In certain embodiments, X2 is —(CH2)4-. In certain embodiments, X2 is —(CH2)5-. In certain embodiments, X2 is —(CH2)6-. In certain embodiments, X2 is —(CH2)7-. In certain embodiments, X2 is —(CH2)8-. In certain embodiments, X2 is —(CH2)9-. In certain embodiments, X2 is —(CH2)10-.

In certain embodiments, X3 is an optionally substituted C1-C12 alkylene. In certain embodiments, X3 is an optionally substituted C1-C12 alkenylene. In certain embodiments, X3 is an optionally substituted C1-C10 aliphatic. In certain embodiments, X3 is an optionally substituted C1-C10 alkylene. In certain embodiments, X3 is an optionally substituted C1-C10 alkenylene. In certain embodiments, X3 is an optionally substituted C1-C8 aliphatic. In certain embodiments, X3 is an optionally substituted C1-C8 alkylene. In certain embodiments, X3 is an optionally substituted C1-C8 alkenylene. In certain embodiments, X3 is an optionally substituted C1-C6 aliphatic. In certain embodiments, X3 is an optionally substituted C1-C6 alkylene. In certain embodiments, X3 is an optionally substituted C1-C6 alkenylene. In certain embodiments, X3 is an optionally substituted C2-C12 aliphatic. In certain embodiments, X3 is an optionally substituted C2-C12 alkylene. In certain embodiments, X3 is an optionally substituted C2-C12 alkenylene. In certain embodiments, X3 is an optionally substituted C4-C12 aliphatic. In certain embodiments, X3 is an optionally substituted C4-C12 alkylene. In certain embodiments, X3 is an optionally substituted C4-C12 alkenylene. In certain embodiments, X3 is an optionally substituted C4-C10 aliphatic. In certain embodiments, X3 is an optionally substituted C4-C10 alkylene. In certain embodiments, X3 is an optionally substituted C4-C10 alkenylene. In certain embodiments, X3 is an optionally substituted C6-C8 aliphatic. In certain embodiments, X3 is an optionally substituted C6-C8 alkylene. In certain embodiments, X3 is an optionally substituted C6-C8 alkenylene. In certain embodiments, X3 is —(CH2)-. In certain embodiments, X3 is —(CH2)2-. In certain embodiments, X3 is —(CH2)3-. In certain embodiments, X3 is —(CH2)4-. In certain embodiments, X3 is —(CH2)5-. In certain embodiments, X3 is —(CH2)6-. In certain embodiments, X3 is —(CH2)7-. In certain embodiments, X3 is —(CH2)8-. In certain embodiments, X3 is —(CH2)9-. In certain embodiments, X3 is —(CH2)10-.

In certain embodiments, X² and X³ are both —(CH₂)₈—. In certain embodiments, X² and X³ are both —(CH₂)₆—.

### X⁴

As disclosed in Formula (AT), in certain embodiments, X⁴ is a bond or C₂-C₆ aliphatic. In certain embodiments, X⁴ is a bond. In certain embodiments, X⁴ is C₂-C₆ aliphatic. In certain embodiments, X⁴ is C₂ aliphatic. In certain embodiments, X⁴ is C₃ aliphatic. In certain embodiments, X⁴ is C₄ aliphatic. In certain embodiments, X⁴ is C₅ aliphatic. In certain embodiments, X⁴ is C₆ aliphatic.

### Y and Y²

As disclosed in Formula (AT), in certain embodiments, Y¹ and Y² are each independently

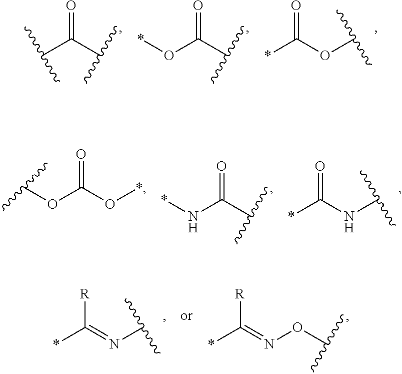

wherein the bond marked with an “*” is attached to X² for Y¹ or X³ for Y². In certain embodiments, Y¹ and Y² are the same. In certain embodiments, Y¹ and Y² are different.

In certain embodiments, Y¹ and Y² are each independently

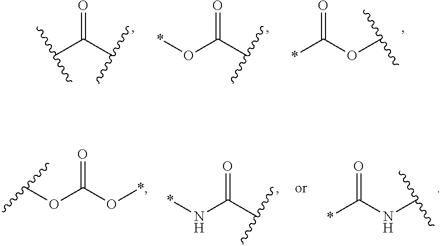

In certain embodiments, Y¹ and Y² are each independently

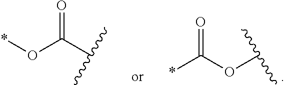 [SMILES: *OC(*)=O ; *OC(*)=O]

In certain embodiments, Y¹ is

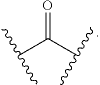 [SMILES: *C(*)=O]

In certain embodiments, Y¹ is

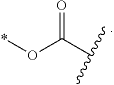 [SMILES: *OC(*)=O]

In certain embodiments, Y¹ is

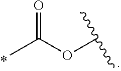

In certain embodiments, Y¹ is

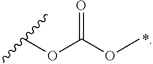

In certain embodiments, Y¹ is

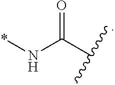 [SMILES: *NC(*)=O]

In certain embodiments, Y¹ is

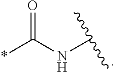 [SMILES: *NC(*)=O]

In certain embodiments, Y¹ is

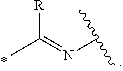

In certain embodiments, Y¹ is

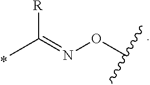

In certain embodiments, Y² is

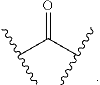 [SMILES: *C(*)=O]

In certain embodiments, Y² is

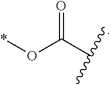 [SMILES: *OC(*)=O]

In certain embodiments, Y² is

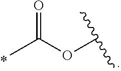

In certain embodiments, Y² is

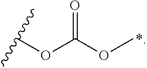

In certain embodiments, Y² is

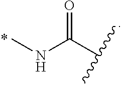

In certain embodiments, Y² is

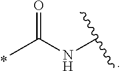

In certain embodiments, Y² is

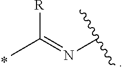

In certain embodiments, Y² is

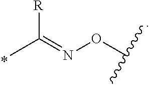

In certain embodiments, Y¹ and Y² are both

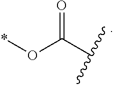 [SMILES: *OC(*)=O]

In certain embodiments, Y¹ and Y² are both

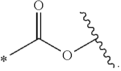

### R²

As disclosed in Formula (AT), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (AT), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different.

### R⁴

As disclosed in Formula (AT), in certain embodiments, R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₅ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷).

In certain embodiments, one of the methylene linkages of R⁴ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

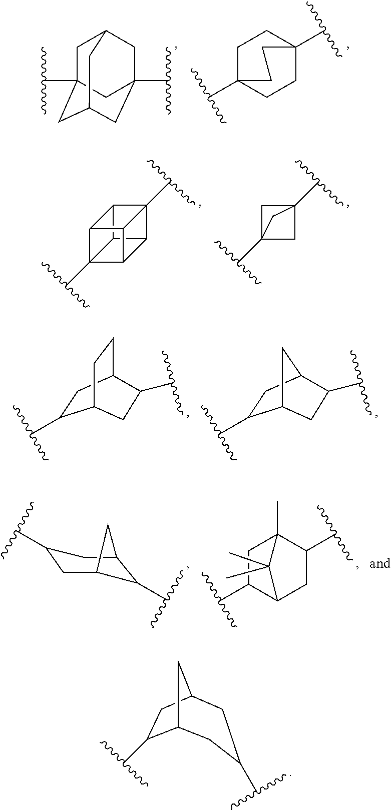

In certain embodiments, R⁴ is selected from is selected from

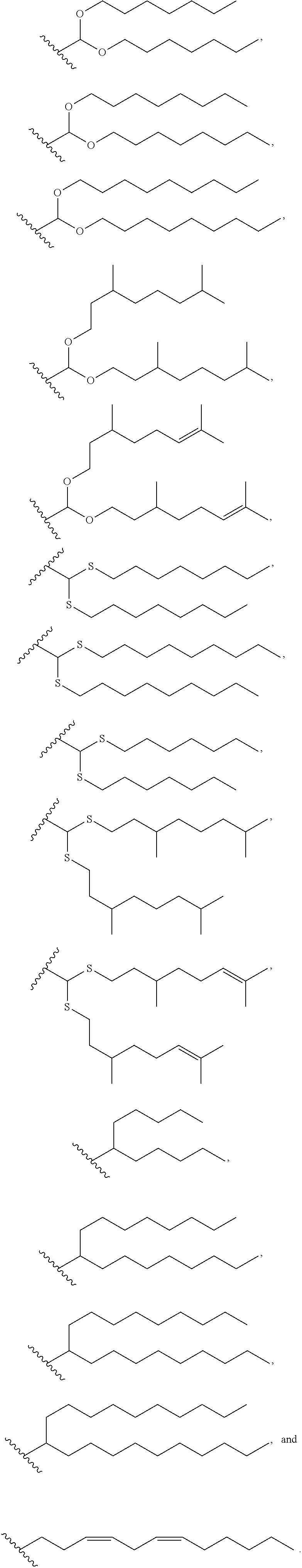

In certain embodiments, R⁴ is selected from is selected from

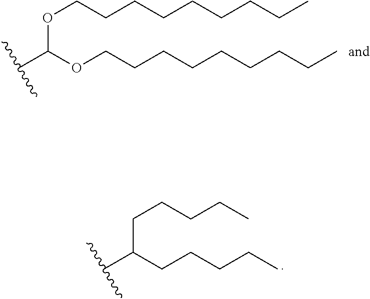 [SMILES: *C(OCCCCCCCCC)OCCCCCCCCC ; "and" ; *C(CCCCC)CCCCC ; "."]

### R⁵

As disclosed in Formula (AT), in certain embodiments, R⁵ is —CH(OR⁸)(OR⁹), —CH(SR⁸)(SR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹).

In certain embodiments, one of the methylene linkages of R⁵ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

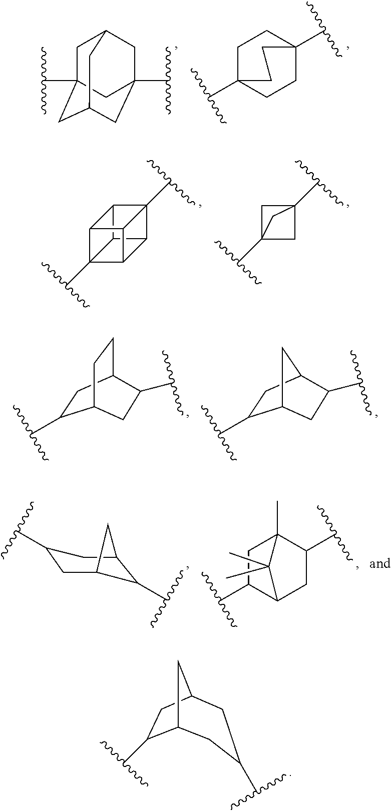

In certain embodiments, R4 and R5 are the same. In certain embodiments, R4 and R5 are different.

In certain embodiments, R⁵ is selected from

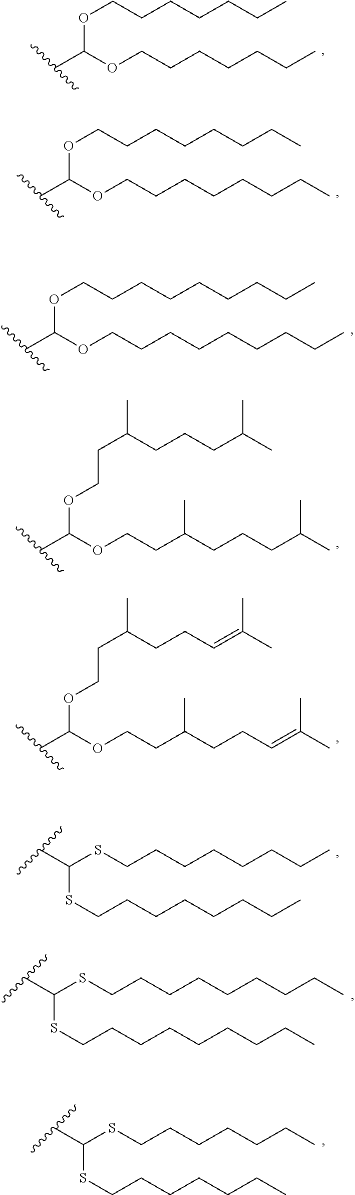

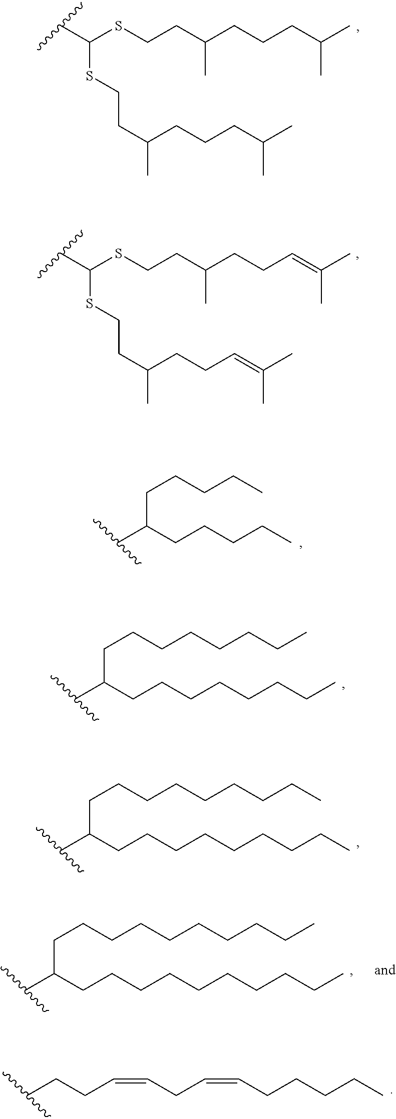

In certain embodiments, R⁵ is selected from is selected from

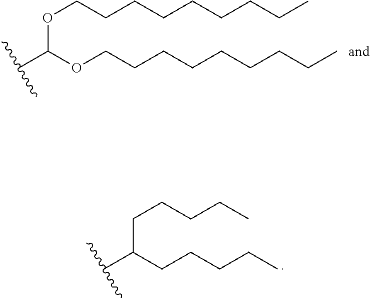 [SMILES: *C(OCCCCCCCCC)OCCCCCCCCC ; "and" ; *C(CCCCC)CCCCC ; "."]

R⁶ and R⁷

As disclosed in Formula (AT), in certain embodiments, R6 and R7 are each independently optionally substituted C1-C14 aliphatic, wherein one or more methylene linkages are each optionally and independently replaced with an optionally substituted C3-C8 cycloalkylenyl, an optionally substituted bridged bicyclic or multicyclic C5-C12 cycloalkylenyl, phenyl, —O—, —NH—, —S—, —SS—, —C(O)—, —OC(O)O—, —OC(O)—, —NHC(O)— or —C(O)O—.

In certain embodiments, R6 and R7 are the same. In certain embodiments, R6 and R7 are different.

In certain embodiments, R6 is optionally substituted C1-C14 aliphatic. In certain embodiments, R6 is optionally substituted C1-C14 alkyl. In certain embodiments, R6 is optionally substituted C1-C14 branched alkyl. In certain embodiments, R6 is optionally substituted C1-C14 straight chain alkyl. In certain embodiments, R6 is optionally substituted C1-C14 alkenylene. In certain embodiments, R6 is optionally substituted C1-C14 branched alkenyl. In certain embodiments, R6 is optionally substituted C1-C14 straight chain alkenyl. In certain embodiments, R6 is optionally substituted C6-C10 alkyl. In certain embodiments, R6 is optionally substituted —(CH2)5CH3. In certain embodiments, R6 is optionally substituted —(CH2)6CH3. In certain embodiments, R6 is optionally substituted —(CH2)7CH3. In certain embodiments, R6 is optionally substituted —(CH2)8CH3. In certain embodiments, R6 is optionally substituted —(CH2)9CH3.

In certain embodiments, one of the methylene linkages of R6 is replaced with an optionally substituted C3-C8 cycloalkylenyl, an optionally substituted bridged bicyclic or multicyclic C5-C12 cycloalkylenyl. In certain embodiments, the optionally substituted bridged bicyclic or multicyclic C₅-C₂₂ cycloalkylenyl is selected from:

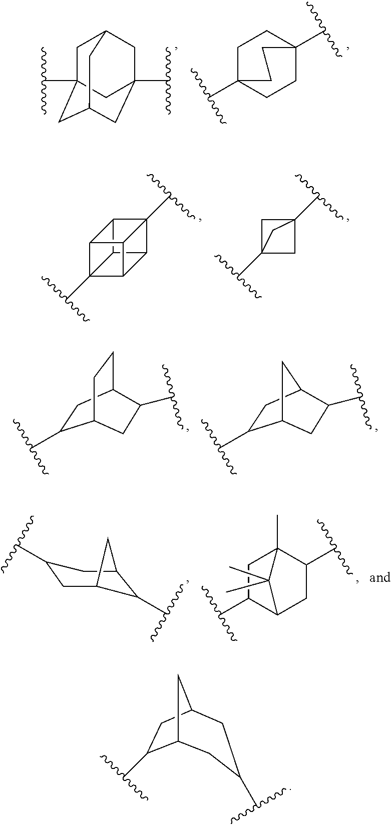

In certain embodiments, R⁷ is optionally substituted C₁-C₁₄ aliphatic. In certain embodiments, R⁷ is optionally substituted C₁-C₁₄ alkyl. In certain embodiments, R⁷ is optionally substituted C₁-C₁₄ branched alkyl. In certain embodiments, R⁷ is optionally substituted C₁-C₁₄ straight chain alkyl. In certain embodiments, R⁷ is optionally substituted C₁-C₁₄ alkenylene. In certain embodiments, R⁷ is optionally substituted C₁-C₁₄ branched alkenyl. In certain embodiments, R⁷ is optionally substituted C₁-C₁₄ straight chain alkenyl. In certain embodiments, R⁷ is optionally substituted C₆-C₁₀ alkyl. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

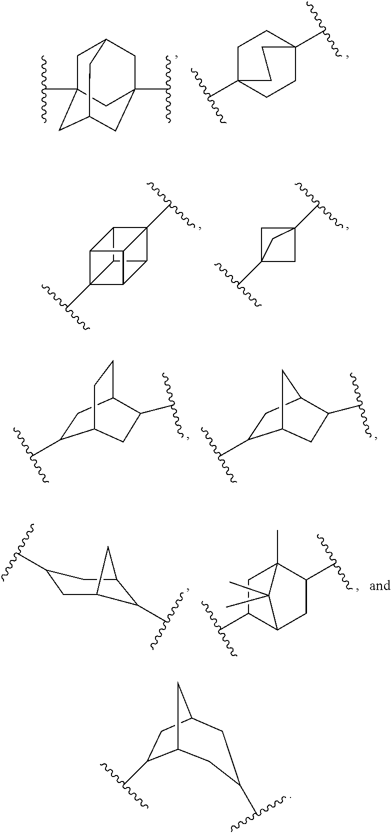

In certain embodiments, each R⁶ and R⁷ are selected from

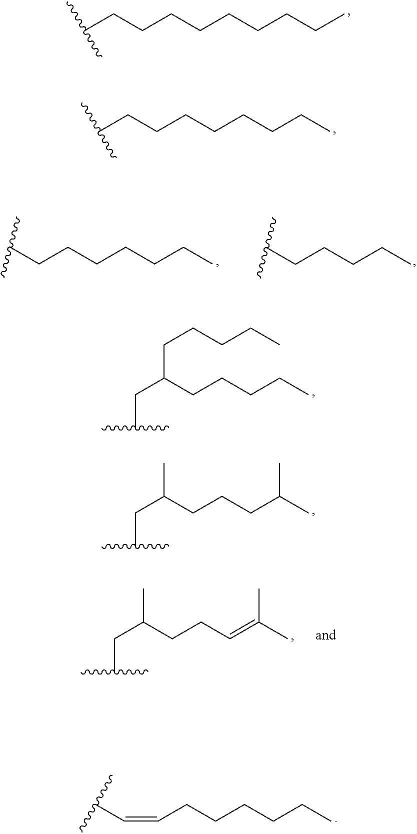

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

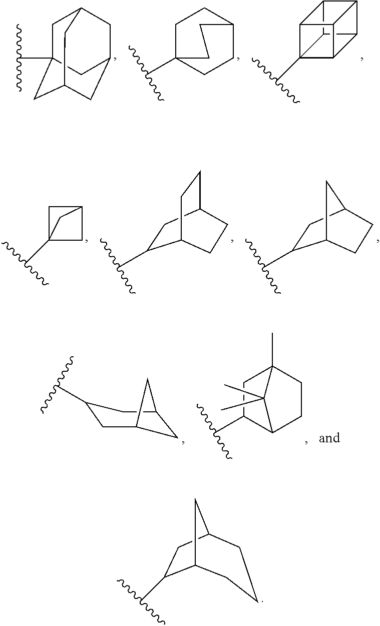

In certain embodiments, the substituted bridged bicyclic or multicyclic C₅-C₁₂ cycloalkylenyl is a structure selected from

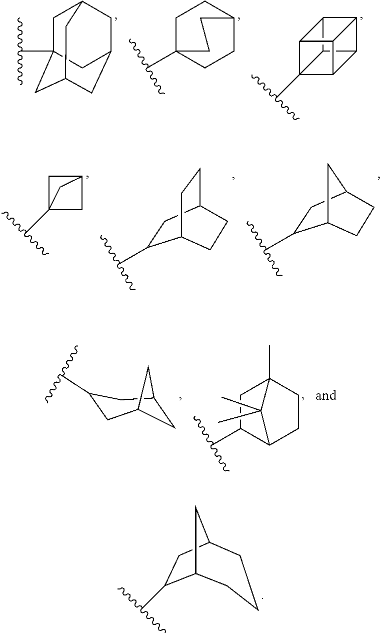

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

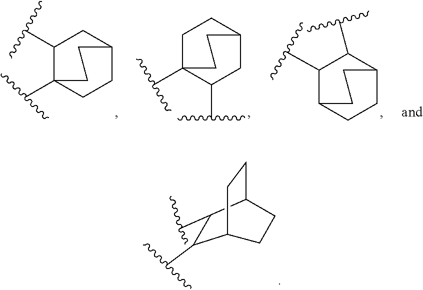

R⁸ and R⁹

As disclosed in Formula (AT), in certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkyl. In certain embodiments, R¹ is optionally substituted C₁-C₁₄ branched alkyl. In certain embodiments, R⁸ is optionally substituted C₁-C₁₄ straight chain alkyl. In certain embodiments, R¹ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R¹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

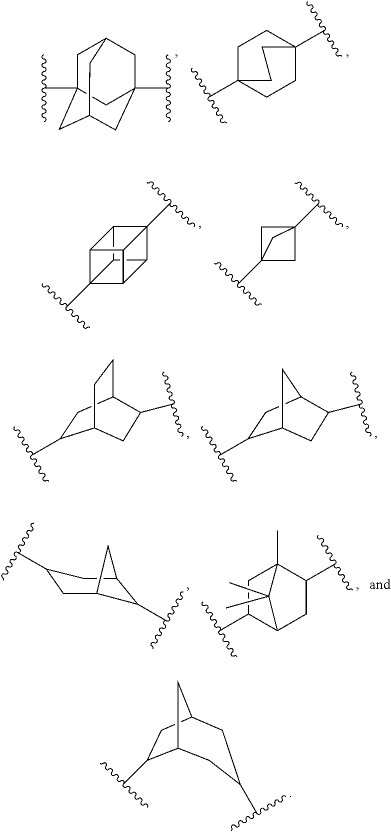

In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

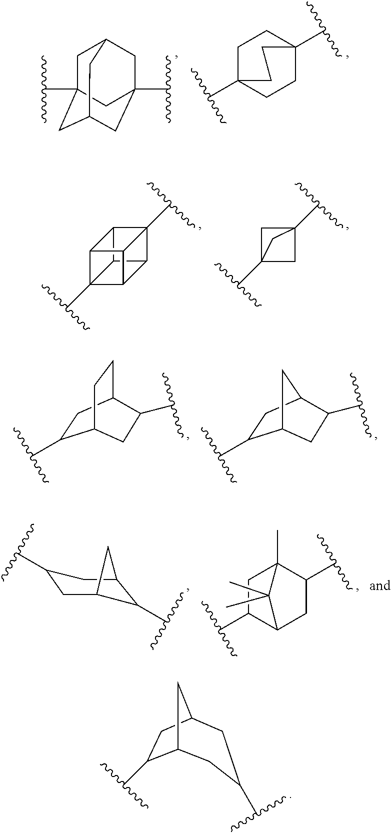

In certain embodiments, each R⁸ and R⁹ are selected from

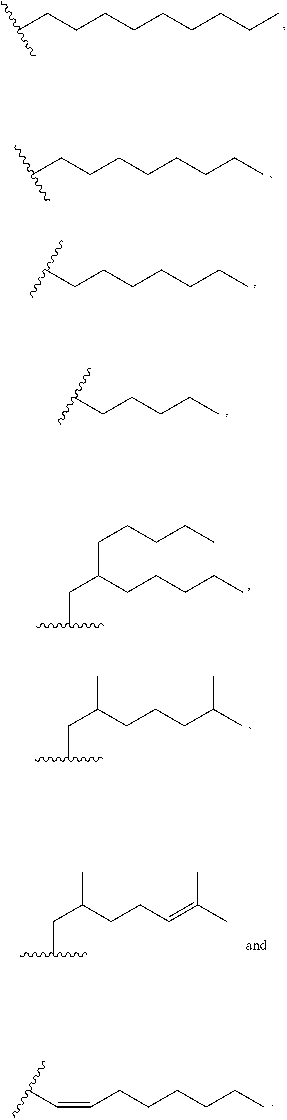

In some embodiments, R⁸ and R⁹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R⁸ and R⁹ are each independently selected from an optionally substituted bridged bicyclic C₅-C₁₂ cycloalkylenyl. In certain embodiments, R⁸ is an optionally substituted bridged multicyclic C₅-C₁₂ cycloalkylenyl. In certain embodiments, R⁹ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

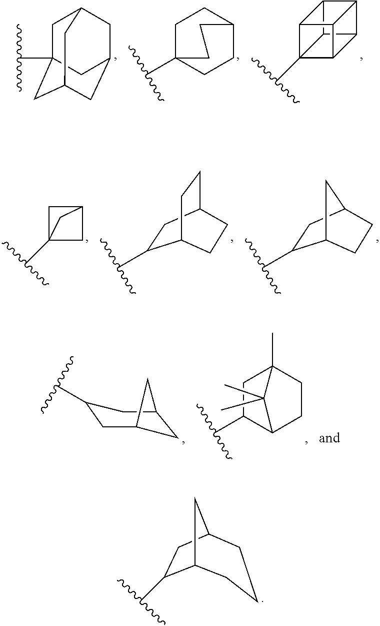

In certain embodiments, the substituted bridged bicyclic or multicyclic C₅-C₁₂ cycloalkylenyl is a structure selected from

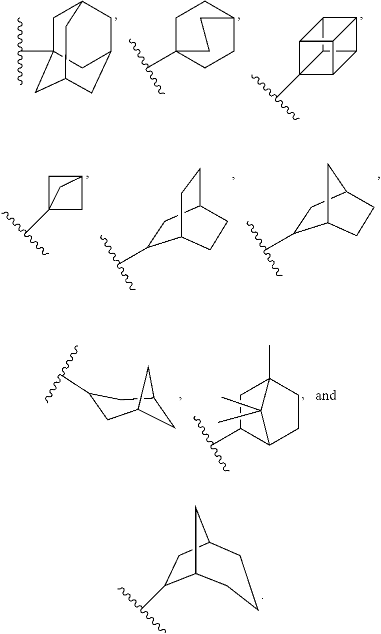

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

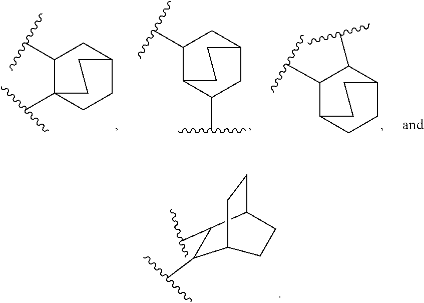 [SMILES: *C1CC2CCC1(*)CC2 ; *C1CC2CCC1C(*)C2 ; *C1C2CCC(CC2)C1* ; *C1C2CCC(CC2)C1*]

In some embodiments, Lipids of the Present Disclosure are selected from any lipid in Table (I-G) below or a pharmaceutically acceptable salt thereof:

TABLE (I-G)
Non-Limiting Examples of Ionizable Lipids of the Present Disclosure

| Cmpd | Structure |
|---|---|
| AT-1 | 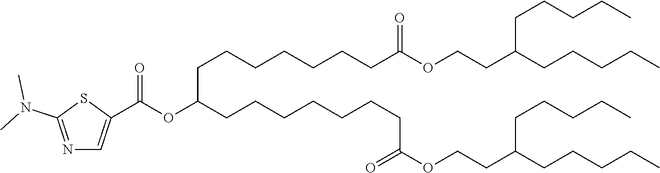 [SMILES: CCCCCC(CCCCC)CCOC(=O)CCCCCCCC(CCCCCCCC(=O)OCCC(CCCCC)CCCCC)OC(=O)c1cnc(N(C)C)s1] |
| AT-2 | 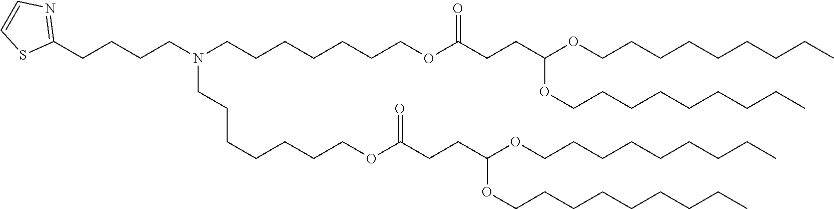 [SMILES: CCCCCCCCCOC(CCC(=O)OCCCCCCCN(CCCCCCCOC(=O)CCC(OCCCCCCCCC)OCCCCCCCCC)CCCCc1nccs1)OCCCCCCCCC] |
| AT-3 | 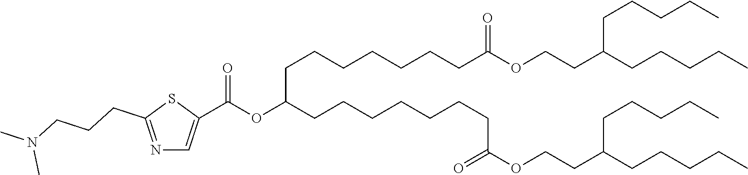 [SMILES: CCCCCC(CCCCC)CCOC(=O)CCCCCCCC(CCCCCCCC(=O)OCCC(CCCCC)CCCCC)OC(=O)c1cnc(CCCN(C)C)s1] |
| AT-4 | 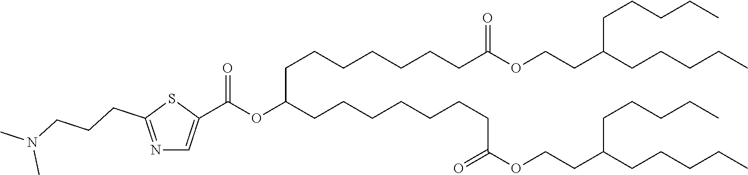 [SMILES: CCCCCC(CCCCC)CCOC(=O)CCCCCCCC(CCCCCCCC(=O)OCCC(CCCCC)CCCCC)OC(=O)c1cnc(CCCN(C)C)s1] |
| AT-5 | 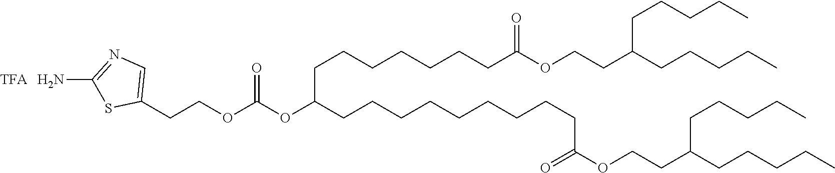 [SMILES: CCCCCC(CCCCC)CCOC(=O)CCCCCCCCCC(CCCCCCCC(=O)OCCC(CCCCC)CCCCC)OC(=O)OCCc1cnc(N)s1 ; "TFA"] |
| AT-6 | 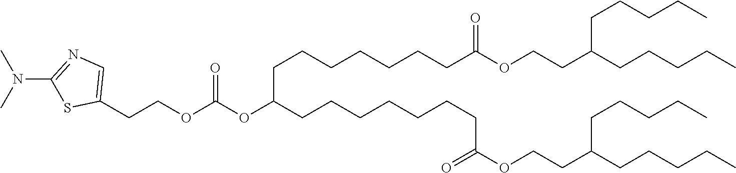 [SMILES: CCCCCC(CCCCC)CCOC(=O)CCCCCCCC(CCCCCCCC(=O)OCCC(CCCCC)CCCCC)OC(=O)OCCc1cnc(N(C)C)s1] |
| AT-7 | 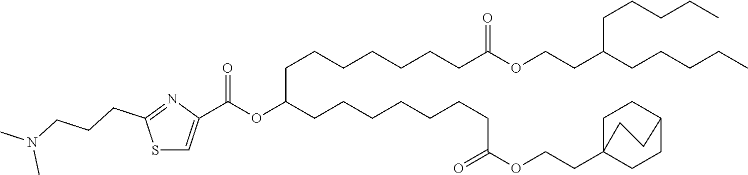 [SMILES: CCCCCC(CCCCC)CCOC(=O)CCCCCCCC(CCCCCCCC(=O)OCCC12CCC(CC1)CC2)OC(=O)c1csc(CCCN(C)C)n1] |
| AT-8 | 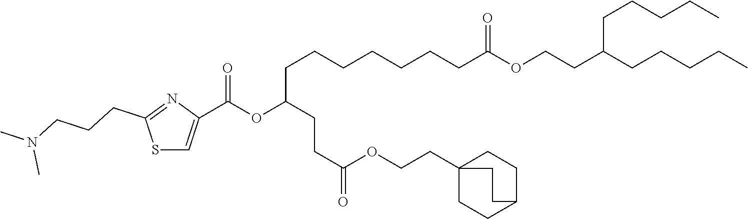 [SMILES: CCCCCC(CCCCC)CCOC(=O)CCCCCCCC(CCC(=O)OCCC12CCC(CC1)CC2)OC(=O)c1csc(CCCN(C)C)n1] |

### Series “AC”

Described below are a number of exemplary ionizable lipids of the present disclosure. In certain embodiments, the ionizable lipid is one selected from those disclosed in PCT Application PCT/US2024/019990, which is incorporated by reference herein, in its entirety.

In some embodiments, ionizable lipids of the present disclosure have a structure of Formula (AC)

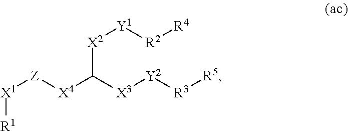

or a pharmaceutically acceptable salt thereof, wherein:
R¹ is selected from the group consisting of —NR₂,

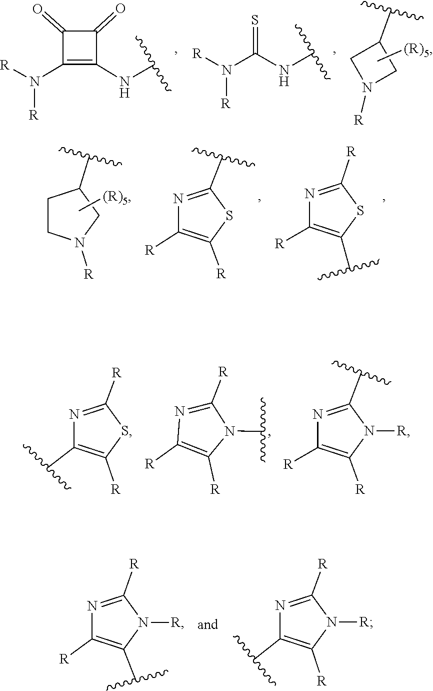

each R is independently —H or C₁-C₆ aliphatic;
X¹ is a bond or optionally substituted C₂-C₆ aliphatic;
Z is

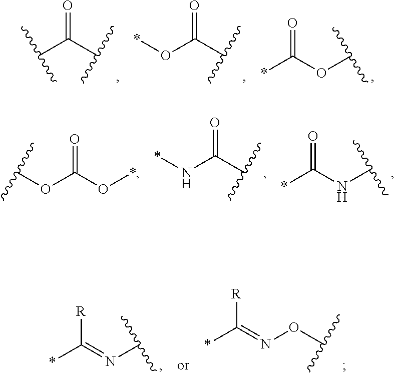

wherein the bond marked with an “*” is attached to X¹;
X² and X³ are each independently optionally substituted C₁-C₁₂ aliphatic;
X⁴ is a bond or C₂-C₆ aliphatic;
Y¹ and Y² are independently selected from the group consisting of

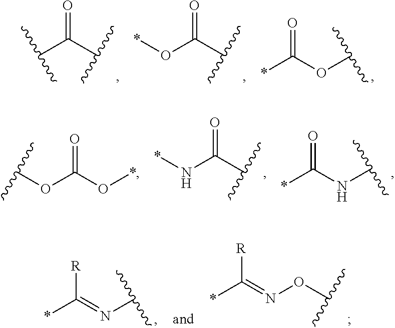

wherein the bond marked with an “*” is attached to X² for Y¹ or X³ for Y²;
R² is optionally substituted C₁-C₆ aliphatic;
R³ is optionally substituted C₁-C₆ aliphatic;
R⁴ is —CH(OR⁶)(OR⁷);
R⁵ is —CH(OR⁸)(OR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

### Additional Formulae

In certain embodiments, ionizable lipids of the present disclosure have a structure of Formula (AC), wherein the ionizable lipids of the present disclosure have a structure of Formula (AC-A), (AC-B), (AC-C), (AC-D), (AC-D1), (AC-D2), (AC-E), (AC-F), (AC-G), (AC-H), or (AC-I):

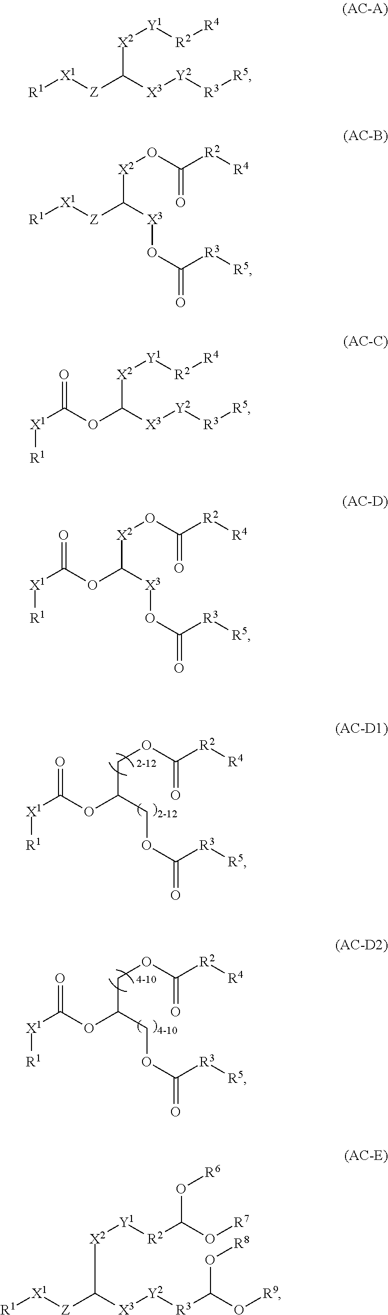

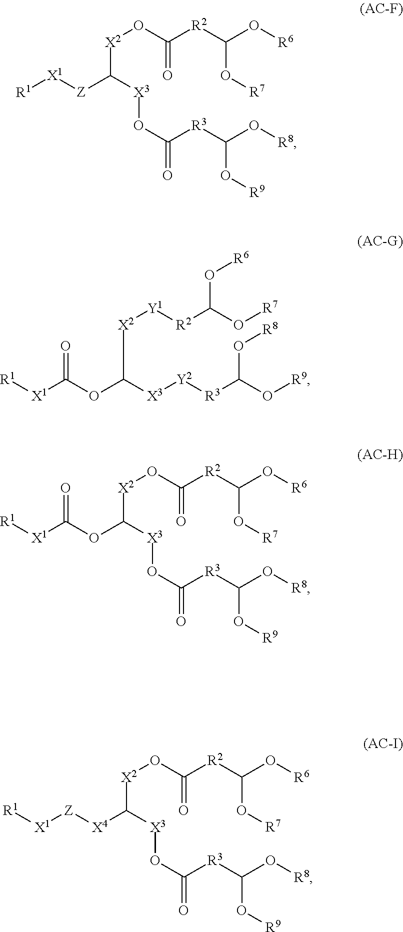

or a pharmaceutically acceptable salt thereof, wherein R¹, R, X¹, Z, X², X³, Y¹, Y², R², R³, R⁴, R⁵, R⁶, R⁷, R⁸, and R⁹ are as described in Formula (AC) or as otherwise described in any embodiments below.

In certain embodiments, R¹ is selected from the group consisting of —NR₂,

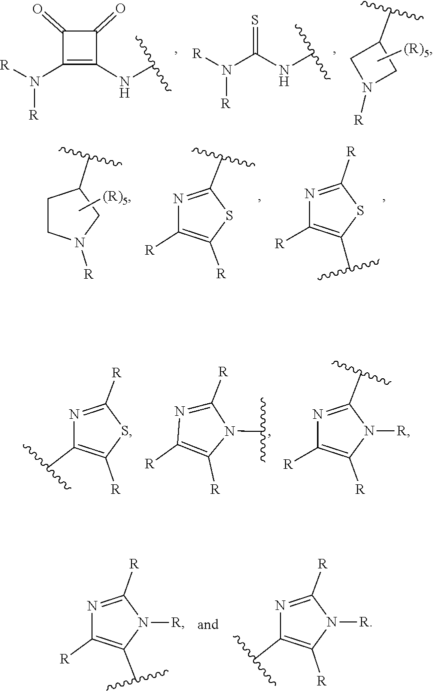

In certain embodiments, R¹ is selected from the group consisting of —NR₂,

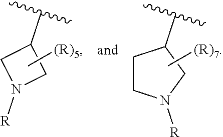

In certain embodiments, R¹ is —NR₂. In certain embodiments, R¹

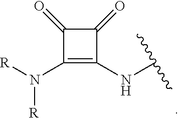

In certain embodiments, R¹ is

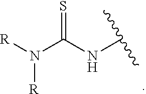

In certain embodiments, R¹ is

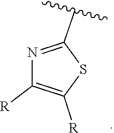

In certain embodiments, R¹ is

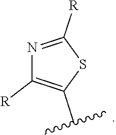

In certain embodiments, R¹ is

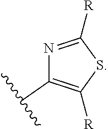

In certain embodiments, R¹ is

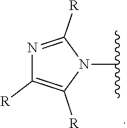

In certain embodiments, R¹ is

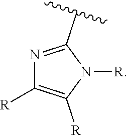

In certain embodiments, R¹ is

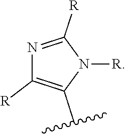

In certain embodiments, R¹ is

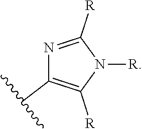

In certain embodiments, R¹ is

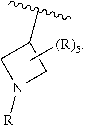

In certain embodiments, R¹ is

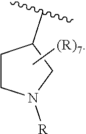

In certain embodiments, R¹ is selected from the group consisting of —N(Et)₂, —N(Me)(Et),

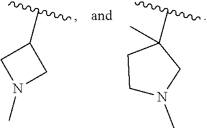

In certain embodiments, R¹ is —N(Et)₂. In certain embodiments, R¹ is —N(Me)₂. In certain embodiments, R¹ is —N(Me)(Et In certain embodiments, R¹ is —NH₂. In certain embodiments, R¹ is —N(nPr)₂. In certain embodiments, R¹ is —N(iPr)₂. In certain embodiments, R¹ is —N(Me)(Et). In certain embodiments, R¹ is

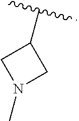 [SMILES: *C1CN(C)C1]

In certain embodiments, R¹ is

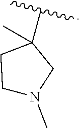 [SMILES: *C1(C)CCN(C)C1]

In certain embodiments, R¹ is

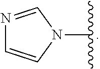 [SMILES: *n1ccnc1]

### X¹

In certain embodiments, X¹ is optionally substituted C₂-C₆ aliphatic. In certain embodiments, X¹ is optionally substituted C₂-C₆ alkylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)₂—. In certain embodiments, X¹ is —(CH₂)₃—. In certain embodiments, X¹ is —(CH₂)₄—. In certain embodiments, X is —(CH₂)₅—. In certain embodiments, X is —(CH₂)₆—. In certain embodiments, X¹ is a bond.

### Z

In certain embodiments, Lipids of the Disclosure have a structure of Formula (AC), (AC-A), (AC-B), (AC-E), (AC-F), or (AC-I), wherein Z is

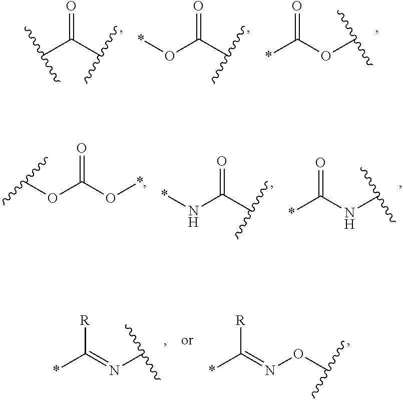

wherein the bond marked with an “*” is attached to X¹. In certain embodiments, Lipids of the Disclosure have a structure of Formula (AC), (AC-A), (AC-B), (AC-E), (AC-F), or (AC-I), Z is

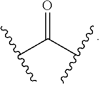 [SMILES: *C(*)=O]

In certain embodiments, Lipids of the Disclosure have a structure of Formula (AC), (AC-A), (AC-B), (AC-E), (AC-F), or (AC-I), Z is

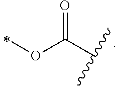

In certain embodiments, Lipids of the Disclosure have a structure of Formula (AC), (AC-A), (AC-B), (AC-E), (AC-F), or (AC-I), Z is

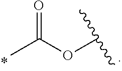 [SMILES: *OC(C)=O]

In certain embodiments, Lipids of the Disclosure have a structure of Formula (AC), (AC-A), (AC-B), (AC-E), (AC-F), or (AC-I), Z is

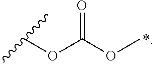

In certain embodiments, Lipids of the Disclosure have a structure of Formula (AC), (AC-A), (AC-B), (AC-E), (AC-F), or (AC-I), Z is

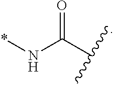 [SMILES: *NC(*)=O]

In certain embodiments, Lipids of the Disclosure have a structure of Formula (AC), (AC-A), (AC-B), (AC-E), (AC-F), or (AC-I), Y¹ is

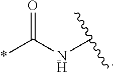 [SMILES: *NC(*)=O]

In certain embodiments, Lipids of the Disclosure have a structure of Formula (AC), (AC-A), (AC-B), (AC-E), (AC-F), or (AC-I), Z is

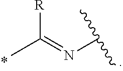

In certain embodiments, Lipids of the Disclosure have a structure of Formula (AC), (AC-A), (AC-B), (AC-E), (AC-F), or (AC-I), Z is

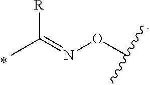

X² and X³

In certain embodiments, X² and X³ are each independently optionally substituted C₁-C₁₂ aliphatic. In certain embodiments, X² and X³ are the same. In certain embodiments, X² and X³ are different.

In certain embodiments, X² is an optionally substituted C₁-C₁₂ alkylene. In certain embodiments, X² is an optionally substituted C₁-C₁₂ alkenylene. I In certain embodiments, X² is an optionally substituted C₁-C₁₀ aliphatic. In certain embodiments, X² is an optionally substituted C₁-C₁₀ alkylene. In certain embodiments, X² is an optionally substituted C₁-C₁₀ alkenylene. In certain embodiments, X² is an optionally substituted C₁-C₈ aliphatic. In certain embodiments, X² is an optionally substituted C₁-C₈ alkylene. In certain embodiments, X² is an optionally substituted C₁-C₈ alkenylene. In certain embodiments, X² is an optionally substituted C₁-C₆ aliphatic. In certain embodiments, X² is an optionally substituted C₁-C₆ alkylene. In certain embodiments, X² is an optionally substituted C₁-C₆ alkenylene. In certain embodiments, X² is an optionally substituted C₂-C₁₂ aliphatic. In certain embodiments, X² is an optionally substituted C₂-C₁₂ alkylene. In certain embodiments, X² is an optionally substituted C₂-C₁₂ alkenylene. In certain embodiments, X² is an optionally substituted C₄-C₁₂ aliphatic. In certain embodiments, X² is an optionally substituted C₄-C₁₂ alkylene. In certain embodiments, X² is an optionally substituted C₄-C₁₂ alkenylene. In certain embodiments, X² is an optionally substituted C₄-C₁₀ aliphatic. In certain embodiments, X² is an optionally substituted C₄-C₁₀ alkylene. In certain embodiments, X² is an optionally substituted C₄-C₁₀ alkenylene. In certain embodiments, X² is an optionally substituted C₆-C₈ aliphatic. In certain embodiments, X² is an optionally substituted C₆-C₈ alkylene. In certain embodiments, X² is an optionally substituted C₆-C₈ alkenylene. In certain embodiments, X² is —(CH₂)—. In certain embodiments, X² is —(CH₂)₂—. In certain embodiments, X² is —(CH₂)₃—. In certain embodiments, X² is —(CH₂)₄—. In certain embodiments, X² is —(CH₂)₅—. In certain embodiments, X² is —(CH₂)₆—. In certain embodiments, X² is —(CH₂)₇—. In certain embodiments, X² is —(CH₂)₅—. In certain embodiments, X² is —(CH₂)₉—. In certain embodiments, X² is —(CH₂)₁₀—.

In certain embodiments, X³ is an optionally substituted C₁-C₁₂ alkylene. In certain embodiments, X³ is an optionally substituted C₁-C₁₂ alkenylene. In certain embodiments, X³ is an optionally substituted C₁-C₁₀ aliphatic. In certain embodiments, X³ is an optionally substituted C₁-C₁₀ alkylene. In certain embodiments, X³ is an optionally substituted C₁-C₁₀ alkenylene. In certain embodiments, X³ is an optionally substituted C₁-C₈ aliphatic. In certain embodiments, X³ is an optionally substituted C₁-C₈ alkylene. In certain embodiments, X³ is an optionally substituted C₁-C₈ alkenylene. In certain embodiments, X³ is an optionally substituted C₁-C₆ aliphatic. In certain embodiments, X³ is an optionally substituted C₁-C₆ alkylene. In certain embodiments, X³ is an optionally substituted C₁-C₆ alkenylene. In certain embodiments, X³ is an optionally substituted C₂-C₁₂ aliphatic. In certain embodiments, X³ is an optionally substituted C₂-C₁₂ alkylene. In certain embodiments, X³ is an optionally substituted C₂-C₁₂ alkenylene. In certain embodiments, X³ is an optionally substituted C₄-C₁₂ aliphatic. In certain embodiments, X³ is an optionally substituted C₄-C₁₂ alkylene. In certain embodiments, X³ is an optionally substituted C₄-C₁₂ alkenylene. In certain embodiments, X³ is an optionally substituted C₄-C₁₀ aliphatic. In certain embodiments, X³ is an optionally substituted C₄-C₁₀ alkylene. In certain embodiments, X³ is an optionally substituted C₄-C₁₀ alkenylene. In certain embodiments, X³ is an optionally substituted C₆-C₈ aliphatic. In certain embodiments, X³ is an optionally substituted C₆-C₈ alkylene. In certain embodiments, X³ is an optionally substituted C₆-C₈ alkenylene. In certain embodiments, X³ is —(CH₂)—. In certain embodiments, X³ is —(CH₂)₂—. In certain embodiments, X³ is —(CH₂)₃—. In certain embodiments, X³ is —(CH₂)₄—. In certain embodiments, X³ is —(CH₂)₅—. In certain embodiments, X³ is —(CH₂)₆—. In certain embodiments, X³ is —(CH₂)₇—. In certain embodiments, X³ is —(CH₂)₈—. In certain embodiments, X³ is —(CH₂)₉—. In certain embodiments, X³ is —(CH₂)₁₀—.

In certain embodiments, X² and X³ are both —(CH₂)₈—. In certain embodiments, X² and X³ are both —(CH₂)₆—.

### X⁴

In certain embodiments, Lipids of the Disclosure have a structure of Formula (AC) or (AC-I), wherein X⁴ is a bond or C₂-C₆ aliphatic. In certain embodiments, Lipids of the Disclosure have a structure of Formula (AC) or (AC-I), wherein X⁴ is a bond. In certain embodiments, Lipids of the Disclosure have a structure of Formula (AC) or (AC-I), wherein X⁴ is C₂-C₆ aliphatic. In certain embodiments, Lipids of the Disclosure have a structure of Formula (AC) or (AC-I), wherein X⁴ is C₂ aliphatic. In certain embodiments, Lipids of the Disclosure have a structure of Formula (AC) or (AC-I), wherein X⁴ is C₃ aliphatic. In certain embodiments, Lipids of the Disclosure have a structure of Formula (AC) or (AC-I), wherein X⁴ is C₄ aliphatic. In certain embodiments, Lipids of the Disclosure have a structure of Formula (AC) or (AC-I), wherein X⁴ is C₅ aliphatic. In certain embodiments, Lipids of the Disclosure have a structure of Formula (AC) or (AC-I), wherein X⁴ is C₆ aliphatic.

Y¹ and Y²

In certain embodiments, Y¹ and Y² are each independently

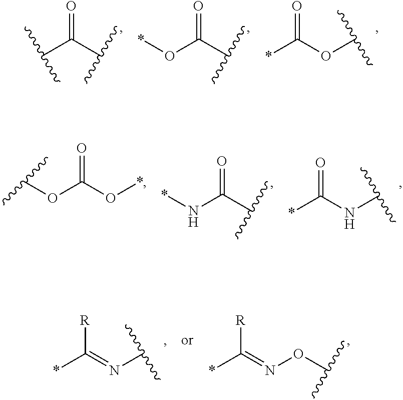

wherein the bond marked with an “*” is attached to X² for Y¹ or X³ for Y². In certain embodiments, Y¹ and Y² are the same. In certain embodiments, Y¹ and Y² are different.

In certain embodiments, Y¹ is

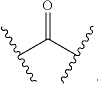 [SMILES: *C(*)=O]

In certain embodiments, Y¹ is

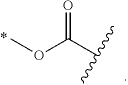 [SMILES: *C(=O)OC]

In certain embodiments, Y¹ is

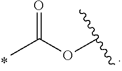 [SMILES: *OC(C)=O]

In certain embodiments, Y¹ is

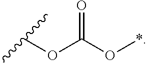

In certain embodiments, Y¹ is

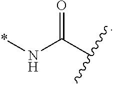 [SMILES: *NC(*)=O]

In certain embodiments, Y¹ is

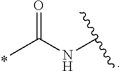

H In certain embodiments, Y¹ is

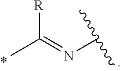

In certain embodiments, Y, is

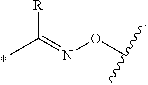

In certain embodiments, Y² is

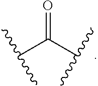 [SMILES: *C(*)=O]

In certain embodiments, Y² is

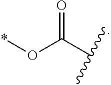 [SMILES: *OC(*)=O]

In certain embodiments, Y² is

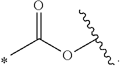 [SMILES: *OC(C)=O]

In certain embodiments, Y² is

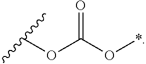

In certain embodiments, Y² is

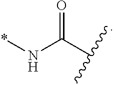 [SMILES: *NC(*)=O]

In certain embodiments, Y² is

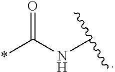 [SMILES: *NC(*)=O]

In certain embodiments, Y² is

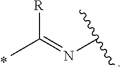

In certain embodiments, Y² is

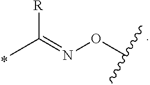

In certain embodiments, Y¹ and Y² are both

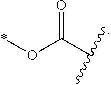 [SMILES: *OC(*)=O]

In certain embodiments, Y¹ and Y² are both

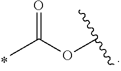 [SMILES: *OC(C)=O]

### R²

In certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

In certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different.

### R⁴

In certain embodiments, R⁴ is —CH(OR⁶)(OR⁷).

In certain embodiments, R⁴ is selected from

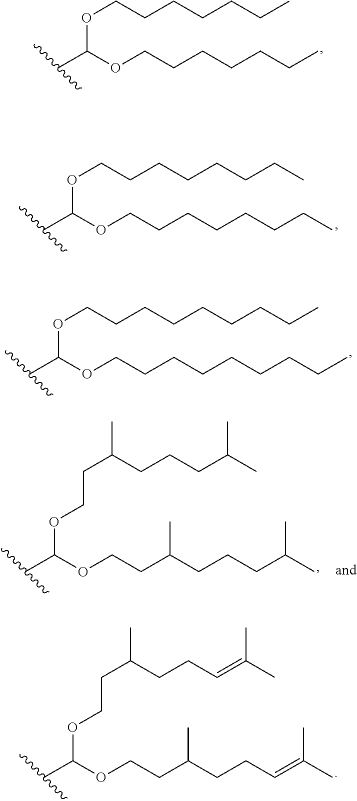 [SMILES: *C(OCCCCCCC)OCCCCCCC ; *C(OCCCCCCCC)OCCCCCCCC ; *C(OCCCCCCCCC)OCCCCCCCCC ; *C(OCCC(C)CCCC(C)C)OCCC(C)CCCC(C)C ; *C(OCCC(C)CCC=C(C)C)OCCC(C)CCC=C(C)C]

### R⁵

In certain embodiments, R⁵ is optionally substituted C₁-C₁₄ aliphatic, —CH(OR⁸)(OR⁹); or —CH(R⁸)(R⁹). In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹).

In certain embodiments, one of the methylene linkages of R⁵ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

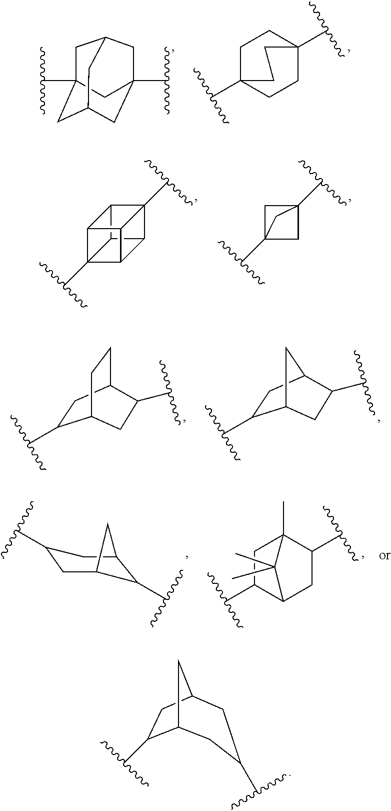

In certain embodiments, R⁴ and R⁵ are the same. In certain embodiments, R⁴ and R⁵ are different.

In certain embodiments, R⁵ is selected from

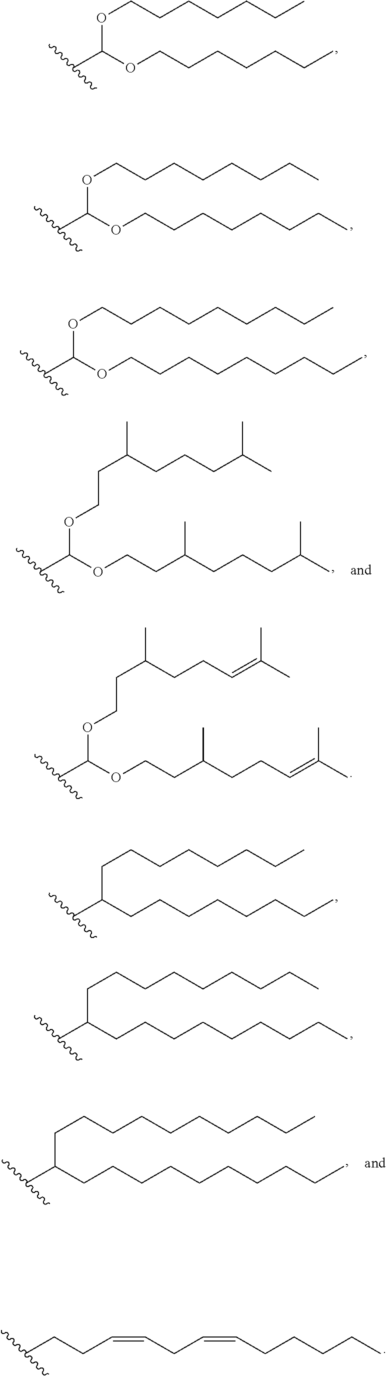

In certain embodiments, R⁵ is selected from

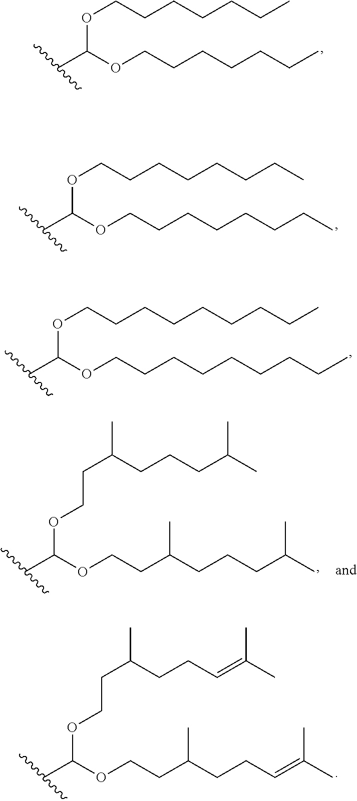 [SMILES: *C(OCCCCCCC)OCCCCCCC ; *C(OCCCCCCCC)OCCCCCCCC ; *C(OCCCCCCCCC)OCCCCCCCCC ; *C(OCCC(C)CCCC(C)C)OCCC(C)CCCC(C)C ; *C(OCCC(C)CCC=C(C)C)OCCC(C)CCC=C(C)C]

R⁶ and R⁷

In certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkylene. In certain embodiments, R⁶ is optionally substituted C₁-C₁₄ branched alkylene. In certain embodiments, R⁶ is optionally substituted C₁-C₁₄ straight chain alkylene. In certain embodiments, R⁶ is optionally substituted C₁-C₁₄ alkenylene. In certain embodiments, R⁶ is optionally substituted C₁-C₁₄ branched alkenylene. In certain embodiments, R⁶ is optionally substituted C₁-C₁₄ straight chain alkenylene. In certain embodiments, R⁶ is optionally substituted C₆-C₁₀ alkylene. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

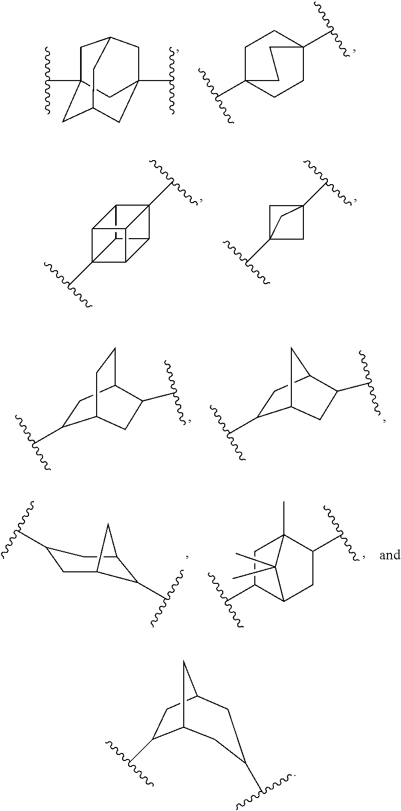

In certain embodiments, R⁷ is optionally substituted C₁-C₁₄ aliphatic. In certain embodiments, R⁷ is optionally substituted C₁-C₁₄ alkylene. In certain embodiments, R⁷ is optionally substituted C₁-C₁₄ branched alkylene. In certain embodiments, R⁷ is optionally substituted C₁-C₁₄ straight chain alkylene. In certain embodiments, R⁷ is optionally substituted C₁-C₁₄ alkenylene. In certain embodiments, R⁷ is optionally substituted C₁-C₁₄ branched alkenylene. In certain embodiments, R⁷ is optionally substituted C₁-C₁₄ straight chain alkenylene. In certain embodiments, R⁷ is optionally substituted C₆-C₁₀ alkylene. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

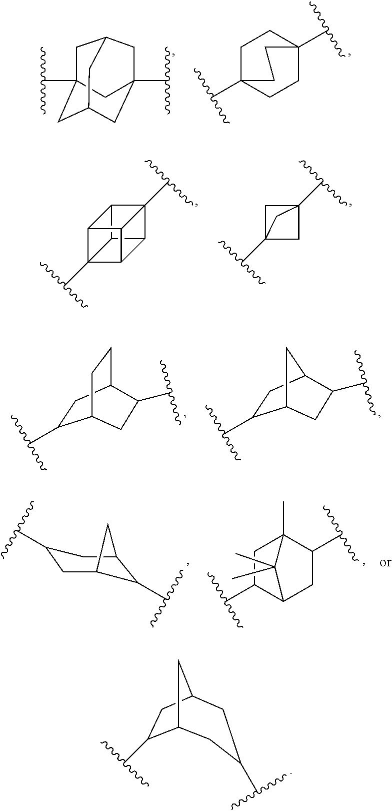

In certain embodiments, R⁶ and R⁷ are selected from

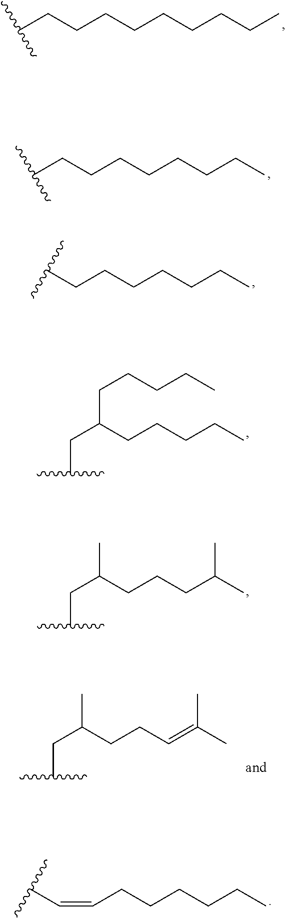

R⁸ and R⁹

In certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkylene. In certain embodiments, R⁸ is optionally substituted C₁-C₁₄ branched alkylene. In certain embodiments, R⁸ is optionally substituted C₁-C₁₄ straight chain alkylene. In certain embodiments, R⁸ is optionally substituted C₁-C₁₄ alkenylene. In certain embodiments, R⁸ is optionally substituted C₁-C₁₄ branched alkenylene. In certain embodiments, R⁸ is optionally substituted C₁-C₁₄ straight chain alkenylene. In certain embodiments, R⁸ is optionally substituted C₆-C₁₀ alkylene. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R¹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

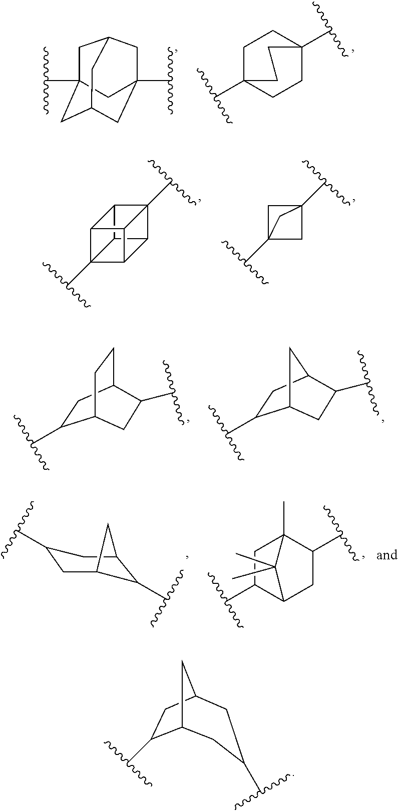

In certain embodiments, R⁹ is optionally substituted C₁-C₁₄ aliphatic. In certain embodiments, R⁹ is optionally substituted C₁-C₁₄ alkylene. In certain embodiments, R⁹ is optionally substituted C₁-C₁₄ branched alkylene. In certain embodiments, R⁹ is optionally substituted C₁-C₁₄ straight chain alkylene. In certain embodiments, R⁹ is optionally substituted C₁-C₁₄ alkenylene. In certain embodiments, R⁹ is optionally substituted C₁-C₁₄ branched alkenylene. In certain embodiments, R⁹ is optionally substituted C₁-C₁₄ straight chain alkenylene. In certain embodiments, R⁹ is optionally substituted C₆-C₁₀ alkylene. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

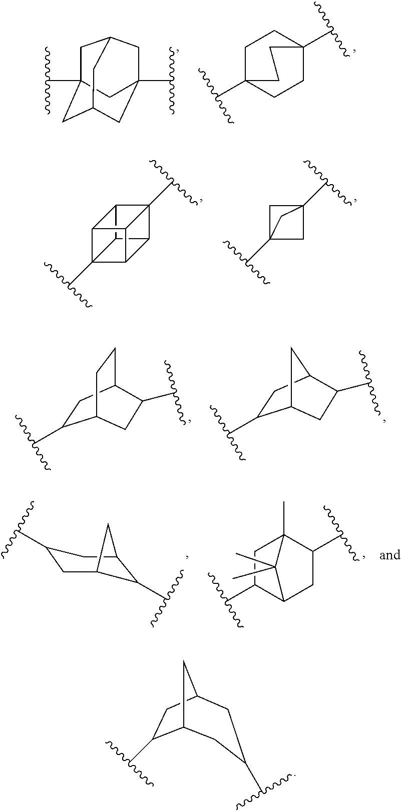

In certain embodiments, R⁸ and R⁹ are selected from

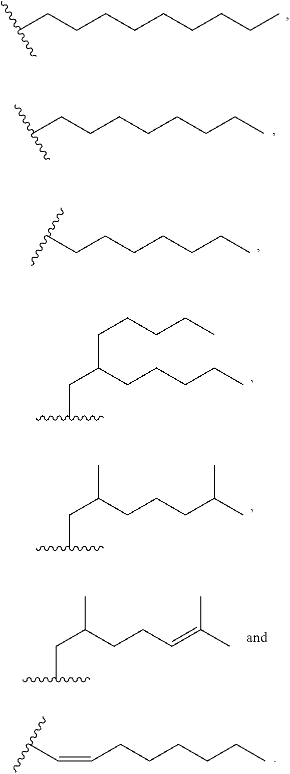

In some embodiments, Lipids of the Present Disclosure are selected from any lipid in Table (I-H) below or a pharmaceutically acceptable salt thereof:

TABLE (I-H)
Non-Limiting Examples of Ionizable Lipids of the Present Disclosure

| Cmpd | Structure |
|---|---|
| AC-1 | 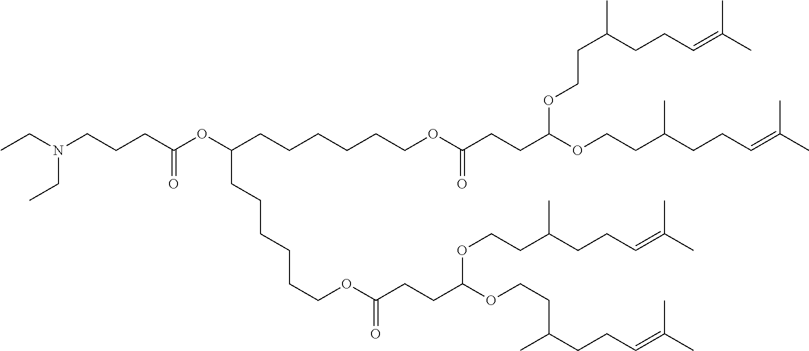 [SMILES: CCN(CC)CCCC(=O)OC(CCCCCCOC(=O)CCC(OCCC(C)CCC=C(C)C)OCCC(C)CCC=C(C)C)CCCCCCOC(=O)CCC(OCCC(C)CCC=C(C)C)OCCC(C)CCC=C(C)C] |
| AC-2 | 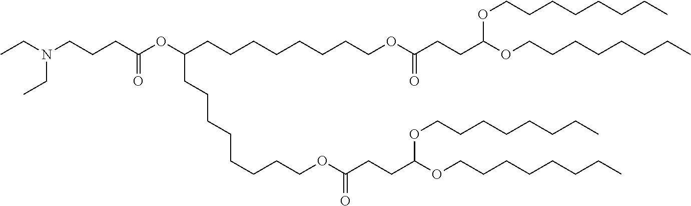 [SMILES: CCCCCCCCOC(CCC(=O)OCCCCCCCCC(CCCCCCCCOC(=O)CCC(OCCCCCCCC)OCCCCCCCC)OC(=O)CCCN(CC)CC)OCCCCCCCC] |
| AC-3 | 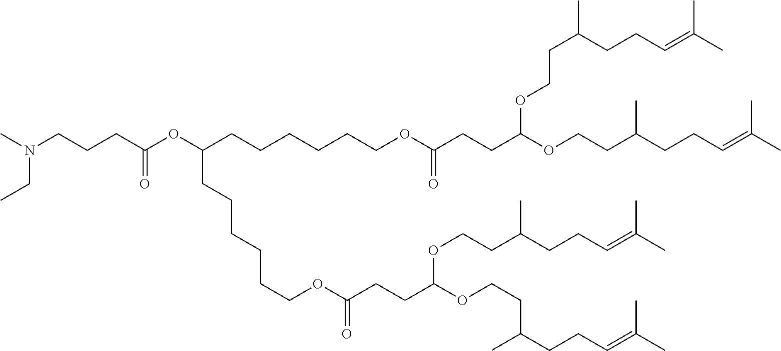 [SMILES: CCN(C)CCCC(=O)OC(CCCCCCOC(=O)CCC(OCCC(C)CCC=C(C)C)OCCC(C)CCC=C(C)C)CCCCCCOC(=O)CCC(OCCC(C)CCC=C(C)C)OCCC(C)CCC=C(C)C] |
| AC-4 | 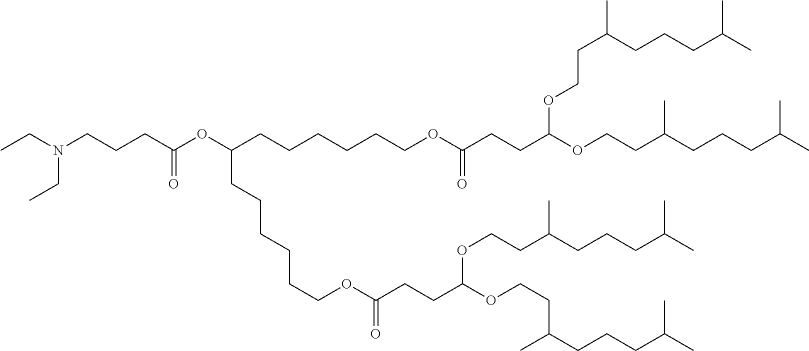 [SMILES: CCN(CC)CCCC(=O)OC(CCCCCCOC(=O)CCC(OCCC(C)CCCC(C)C)OCCC(C)CCCC(C)C)CCCCCCOC(=O)CCC(OCCC(C)CCCC(C)C)OCCC(C)CCCC(C)C] |
| AC-5 | 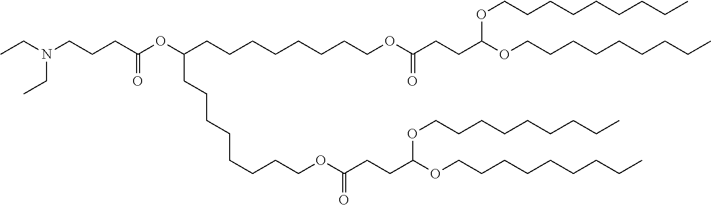 [SMILES: CCCCCCCCCOC(CCC(=O)OCCCCCCCCC(CCCCCCCCOC(=O)CCC(OCCCCCCCCC)OCCCCCCCCC)OC(=O)CCCN(CC)CC)OCCCCCCCCC] |
| AC-6 | 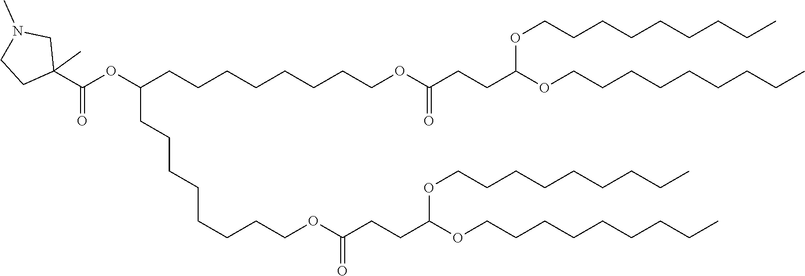 [SMILES: CCCCCCCCCOC(CCC(=O)OCCCCCCCCC(CCCCCCCCOC(=O)CCC(OCCCCCCCCC)OCCCCCCCCC)OC(=O)C1(C)CCN(C)C1)OCCCCCCCCC] |
| AC-7 | 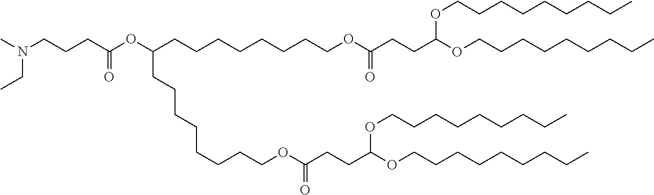 [SMILES: CCCCCCCCCOC(CCC(=O)OCCCCCCCCC(CCCCCCCCOC(=O)CCC(OCCCCCCCCC)OCCCCCCCCC)OC(=O)CCCN(C)CC)OCCCCCCCCC] |
| AC-8 | 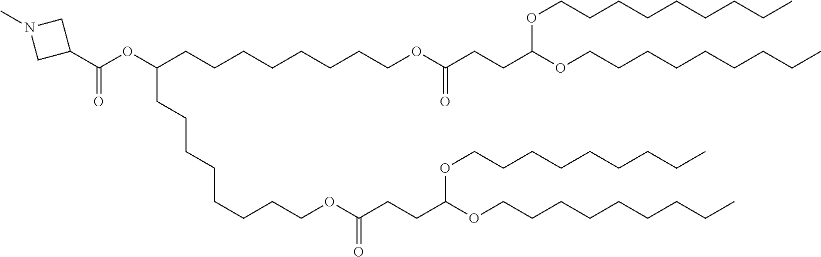 [SMILES: CCCCCCCCCOC(CCC(=O)OCCCCCCCCC(CCCCCCCCOC(=O)CCC(OCCCCCCCCC)OCCCCCCCCC)OC(=O)C1CN(C)C1)OCCCCCCCCC] |
| AC-9 | 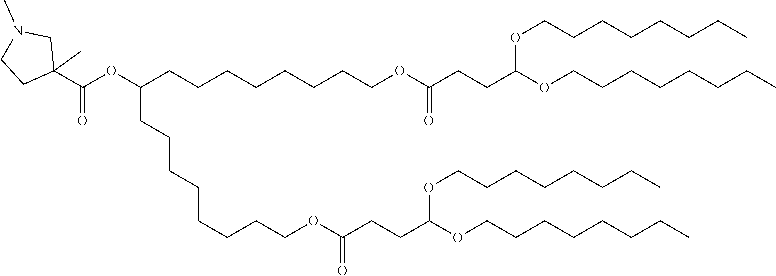 [SMILES: CCCCCCCCOC(CCC(=O)OCCCCCCCCC(CCCCCCCCOC(=O)CCC(OCCCCCCCC)OCCCCCCCC)OC(=O)C1(C)CCN(C)C1)OCCCCCCCC] |
| AC- 10 | 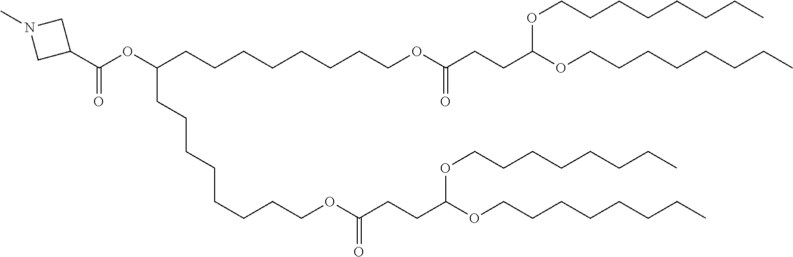 [SMILES: CCCCCCCCOC(CCC(=O)OCCCCCCCCC(CCCCCCCCOC(=O)CCC(OCCCCCCCC)OCCCCCCCC)OC(=O)C1CN(C)C1)OCCCCCCCC] |

### Series “CO”

Described below are a number of exemplary ionizable lipids of the present disclosure. In certain embodiments, the ionizable lipid is one selected from those disclosed in PCT Application PCT/US2024/019990, which is incorporated by reference herein, in its entirety.

The present disclosure provides compound of Formula (CO):

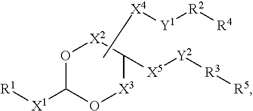

or a pharmaceutically acceptable salt thereof, wherein:
R¹ is selected from the group consisting of —NR₂,

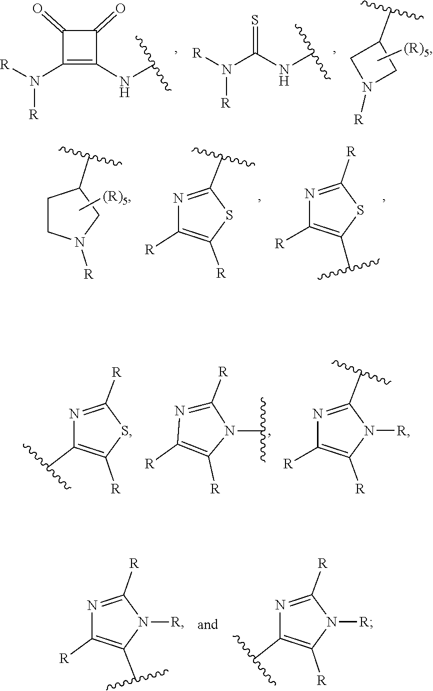

each R is independently —H or C₁-C₆ aliphatic;
X¹ is optionally substituted C₂-C₆ aliphatic, wherein one or more methylene linkages are each optionally and independently replaced with —O—, —NH—, —S—, —SS—, —C(O)—, —OC(O)O—, —OC(O)—, —NHC(O)— or —C(O)O—;
X² is selected from the group consisting of a bond, —CH₂— and —CH₂CH₂—;
X³ is selected from the group consisting of a bond, —CH₂— and —CH₂CH₂—;
X⁴ and X⁵ are each independently optionally substituted C₁-C₁₀ aliphatic;
Y¹ and Y² are each independently

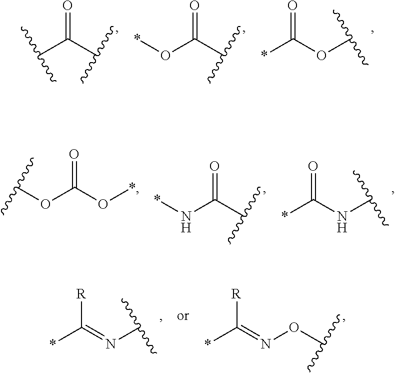

wherein the bond marked with an “*” is attached to X⁴ or X5;
R² is optionally substituted C₁-C₆ aliphatic;
R³ is optionally substituted C₁-C₆ aliphatic;
R⁴ is —CH(OR⁶)(OR⁷); —CH(SR⁶)(SR⁷); —CH(R⁶)(R⁷); or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, the compound of Formula (CO) is a compound of any of the below Formulae:

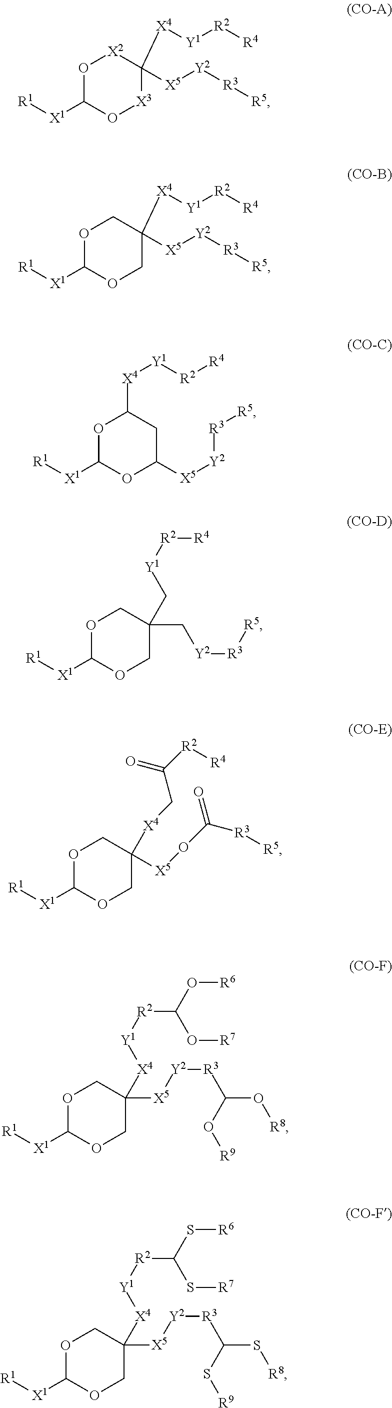

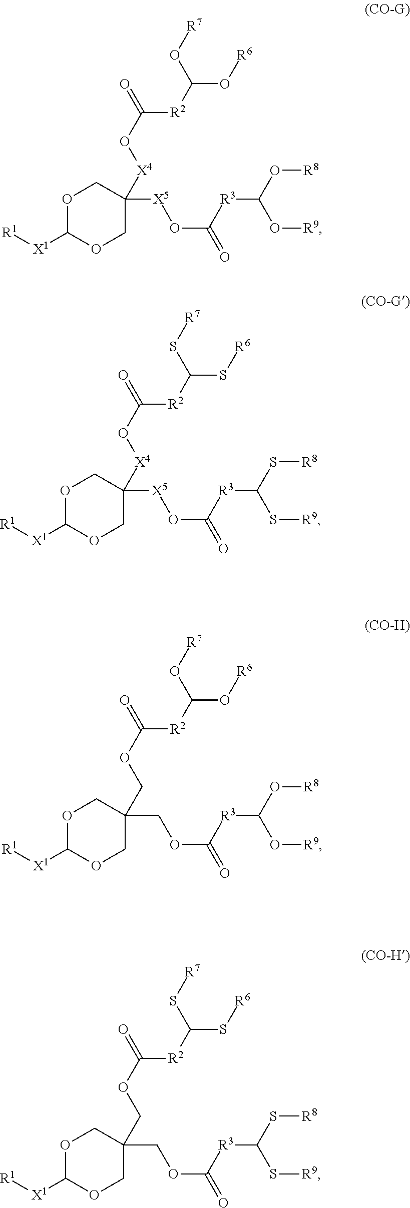

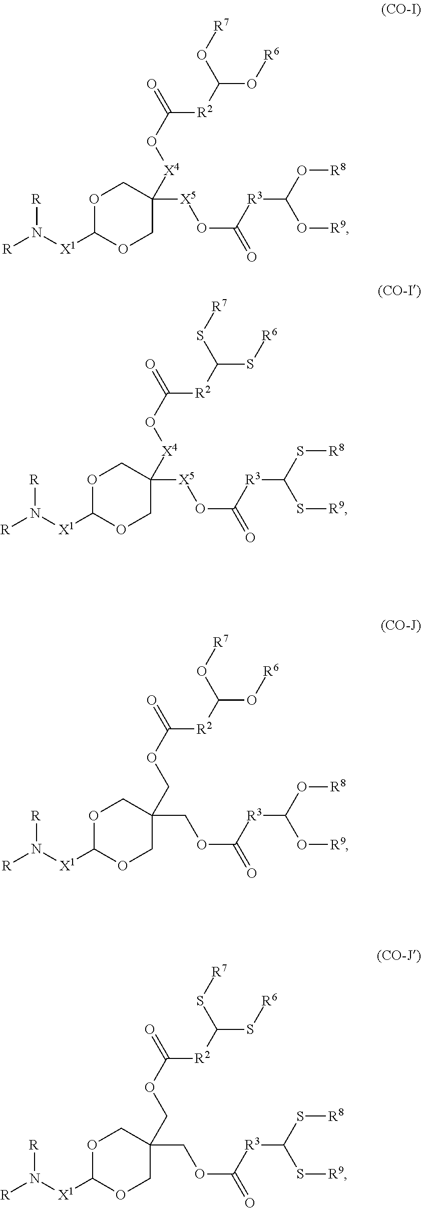

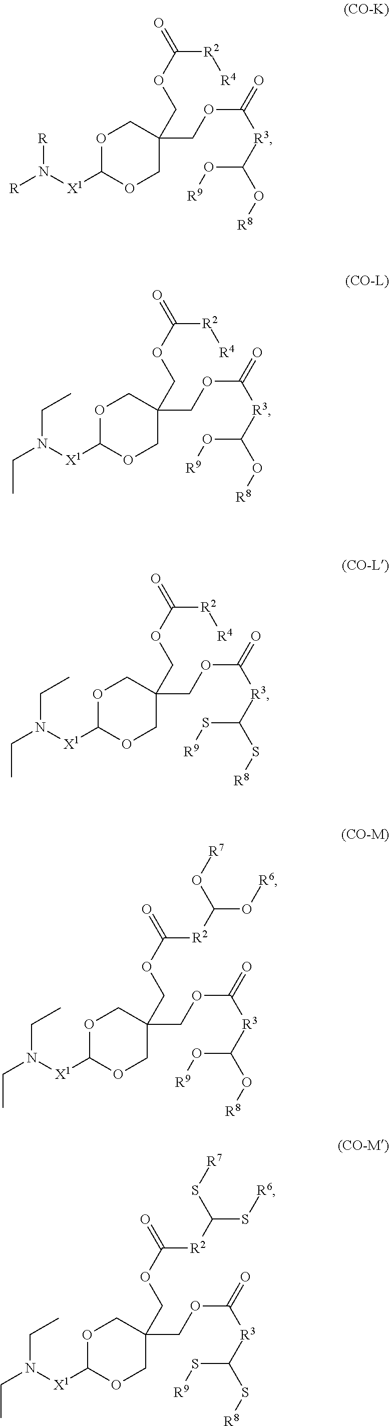

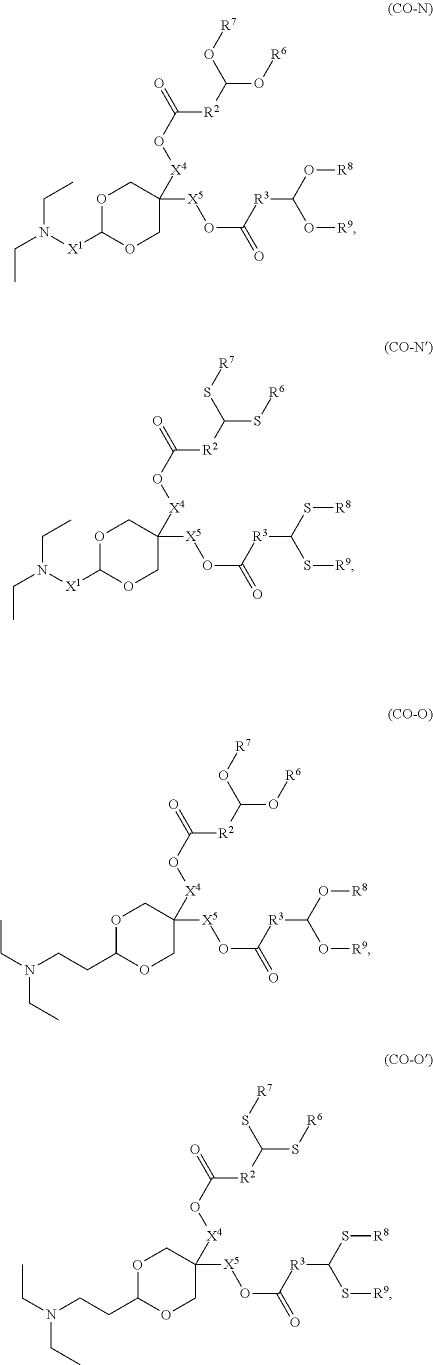

or a pharmaceutically acceptable salt thereof, wherein R¹, R, X¹, X², X³, X⁴, X⁵, Y¹, Y², R², R³, R⁴, R⁵, R⁶, R⁷, R⁸, and R⁹ are as described in Formula (CO) or as otherwise described in any embodiments below.

### R¹

As disclosed in Formula (CO), in certain embodiments, R¹ is selected from the group

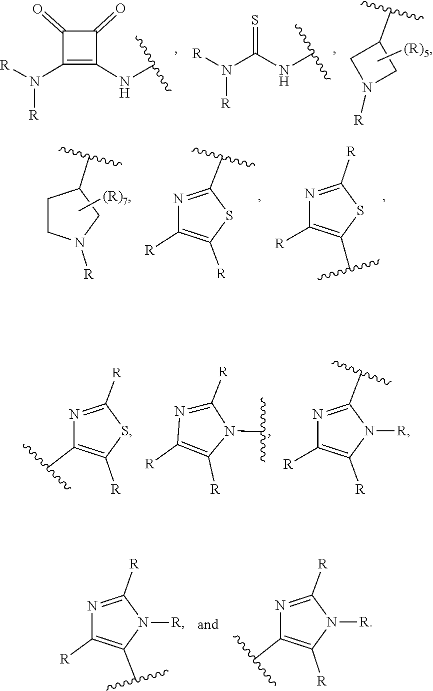

In certain embodiments, R¹ is —NR₂. In certain embodiments, R¹ is

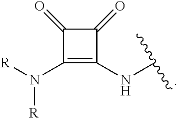

In certain embodiments, R¹ is

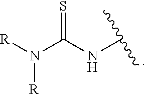

In certain embodiments, R¹ is

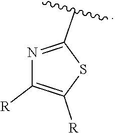

In certain embodiments, R¹ is

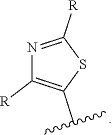

In certain embodiments, R¹ is

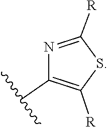

In certain embodiments, R¹ is

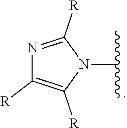

In certain embodiments, R¹ is

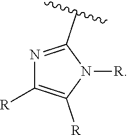

In certain embodiments, R¹ is

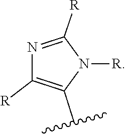

In certain embodiments, R¹ is

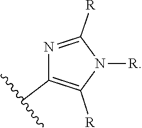

In certain embodiments, R¹ is

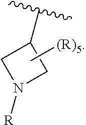

In certain embodiments, R¹ is

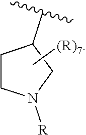

In certain embodiments, R¹ is —N(Et)₂. In certain embodiments, R¹ is —N(Me)₂. In certain embodiments, R1 is —NH₂. In certain embodiments, R¹ is —N(nPr)₂. In certain embodiments, R¹ is —N(iPr)₂. In certain embodiments, R¹ is —N(Me)(Et). In certain embodiments, R¹ is

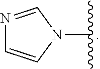 [SMILES: *n1ccnc1]

### X¹

As disclosed in Formula (CO), in certain embodiments, X¹ is optionally substituted C₂-C₆ aliphatic, wherein one or more methylene linkages are each optionally and independently replaced with —O—, —NH—, —S—, —SS—, —C(O)—, —OC(O)O—, —NHC(O)— or —C(O)O—. In certain embodiments, X¹ is optionally substituted C₂-C₆ aliphatic. In certain embodiments, X¹ is optionally substituted C₂-C₆ alkylene. In certain embodiments, X¹ is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### X²

As disclosed in Formula (CO), in certain embodiments, X² is selected from the group consisting of a bond, —CH₂— and —CH₂CH₂—. In certain embodiments, X² is a bond. In certain embodiments, X² is —CH₂—. In certain embodiments, X² is —CH₂CH₂—.

### X³

As disclosed in Formula (CO), in certain embodiments, X³ is selected from the group consisting of a bond, —CH₂— and —CH₂CH₂—. In certain embodiments, X³ is a bond. In certain embodiments, X³ is —CH₂—. In certain embodiments, X³ is —CH₂CH₂—. In certain embodiments, both X² and X³ are —CH₂—. In certain embodiments, both X² and X³ are —CH₂CH₂—. In certain embodiments, X² is a bond and X³ is —CH₂—. In certain embodiments, X² is a bond and X³ is —CH₂CH₂—. In certain embodiments, X³ is a bond and X² is —CH₂—. In certain embodiments, X³ is a bond and X² is —CH₂CH₂—.

X⁴ and X⁵

As disclosed in Formula (CO), in certain embodiments, X⁴ and X⁵ are each independently optionally substituted C₁-C₁₀ aliphatic. In certain embodiments, X⁴ and X⁵ are the same. In certain embodiments, X⁴ and X⁵ are different.

In certain embodiments, X⁴ is an optionally substituted C₁-C₁₀ alkylene. In certain embodiments, X⁴ is an optionally substituted C₁-C₁₀ alkenylene. In certain embodiments, X⁴ is an optionally substituted C₁-C₆ alkylene. In certain embodiments, X⁴ is an optionally substituted C₁-C₆ alkenylene. In certain embodiments, X⁴ is —(CH₂)—. In certain embodiments, X⁴ is —(CH₂)₂—. In certain embodiments, X⁴ is —(CH₂)₃—. In certain embodiments, X⁴ is —(CH₂)₄—. In certain embodiments, X⁴ is —(CH₂)₅—. In certain embodiments, X⁴ is —(CH₂)₆—.

In certain embodiments, X⁵ is an optionally substituted C₁-C₁₀ alkylene. In certain embodiments, X⁵ is an optionally substituted C₁-C₁₀ alkenylene. In certain embodiments, X⁵ is an optionally substituted C₁-C₆ alkylene. In certain embodiments, X⁵ is an optionally substituted C₁-C₆ alkenylene. In certain embodiments, X⁵ is —(CH₂)—. In certain embodiments, X⁵ is —(CH₂)₂—. In certain embodiments, X⁵ is —(CH₂)₃—. In certain embodiments, X⁵ is —(CH₂)₄—. In certain embodiments, X⁵ is —(CH₂)₅—. In certain embodiments, X⁵ is —(CH₂)₆—.

In certain embodiments, X⁴ and X⁵ are both —(CH₂)—. In certain embodiments, X⁴ and X⁵ are both —(CH₂)₂—.

Y¹ and Y²

As disclosed in Formula (CO), in certain embodiments, Y¹ and Y² are each independently

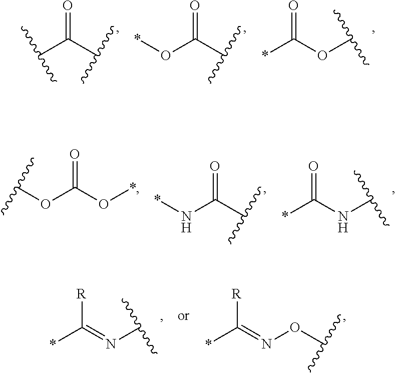

wherein the bond marked with an “*” is attached to X⁴ or X⁵. In certain embodiments, Y¹ and Y² are the same. In certain embodiments, Y¹ and Y² are different.

In certain embodiments, Y¹ is

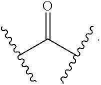 [SMILES: *C(*)=O]

In certain embodiments, ^{Y} is

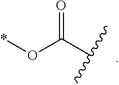 [SMILES: *OC(*)=O]

In certain embodiments, Y¹ is

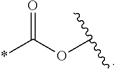 [SMILES: *OC(*)=O]

In certain embodiments, Y¹ is

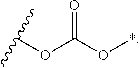

In certain embodiments, Y¹ is

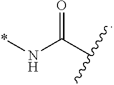 [SMILES: *NC(*)=O]

In certain embodiments, Y¹ is

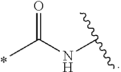

In certain embodiments, Y¹ is

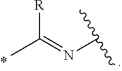

In certain embodiments, Y¹ is

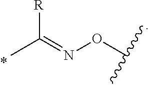

In certain embodiments, Y² is

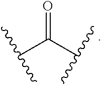 [SMILES: *C(*)=O]

In certain embodiments, Y² is

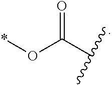 [SMILES: *OC(*)=O]

In certain embodiments, Y² is

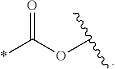 [SMILES: *OC(*)=O]

In certain embodiments, Y² is

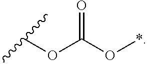

In certain embodiments, Y² is

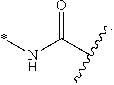 [SMILES: *NC(*)=O]

In certain embodiments, Y² is

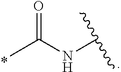

In certain embodiments, Y² is

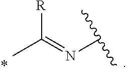

In certain embodiments, Y²
is

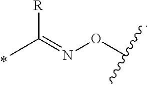

In certain embodiments, Y¹ and Y² are both

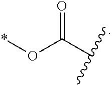 [SMILES: *OC(*)=O]

In certain embodiments, Y¹ and Y² are both

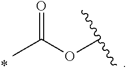 [SMILES: *OC(C)=O]

### R²

As disclosed in Formula (CO), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (CO), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different. In certain embodiments, R² and R³ are both —(CH₂)₂—.

### R⁴

As disclosed in Formula (CO), in certain embodiments, R⁴ is —CH(OR⁶)(OR⁷); —CH(SR⁶)(SR⁷); —CH(R⁶)(R⁷); or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷).

In certain embodiments, R⁴ is selected from N

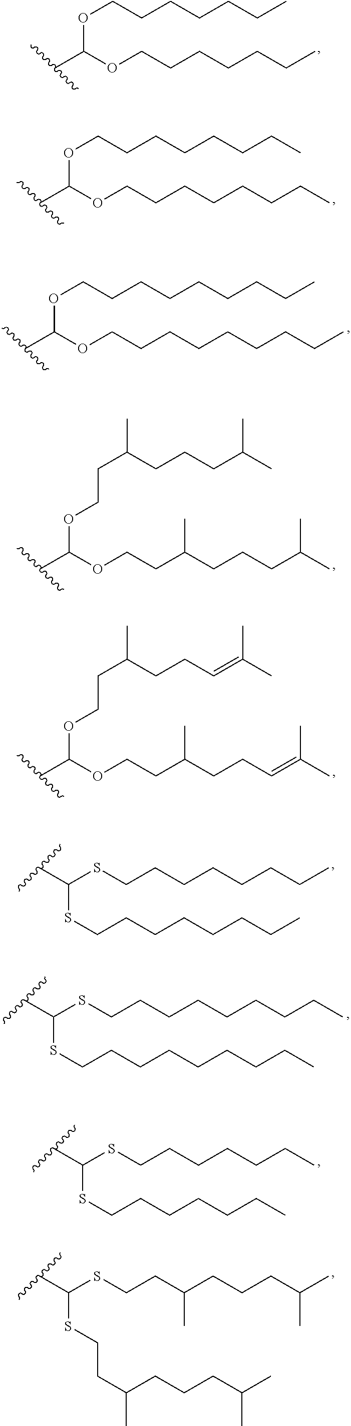

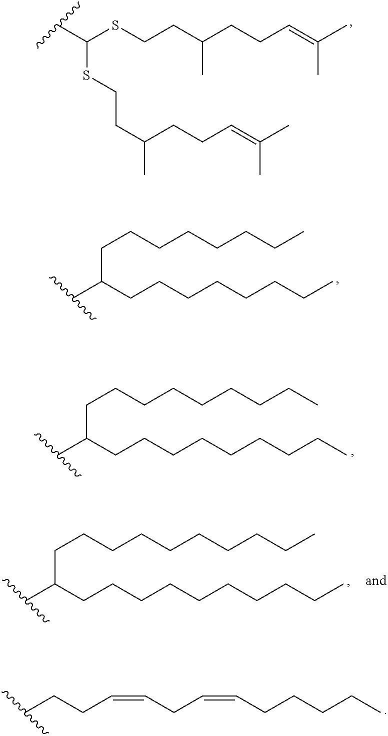 [SMILES: *C(SCCC(C)CCC=C(C)C)SCCC(C)CCC=C(C)C ; *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁴ is selected from

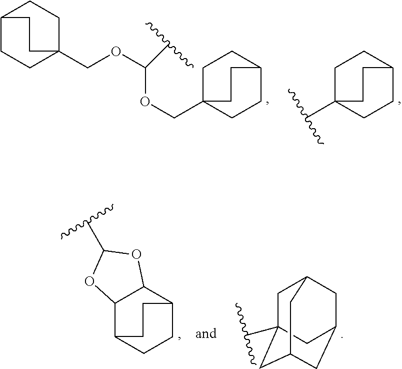 [SMILES: *C(OCC12CCC(CC1)CC2)OCC12CCC(CC1)CC2 ; *C12CCC(CC1)CC2 ; *C1OC2C3CCC(CC3)C2O1 ; *C12CC3CC(CC(C3)C1)C2]

### R⁵

As disclosed in Formula (CO), in certain embodiments, R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹).

In certain embodiments, R⁴ and R⁵ are the same. In certain embodiments, R⁴ and R⁵ are different.

In certain embodiments, R⁵ is selected from

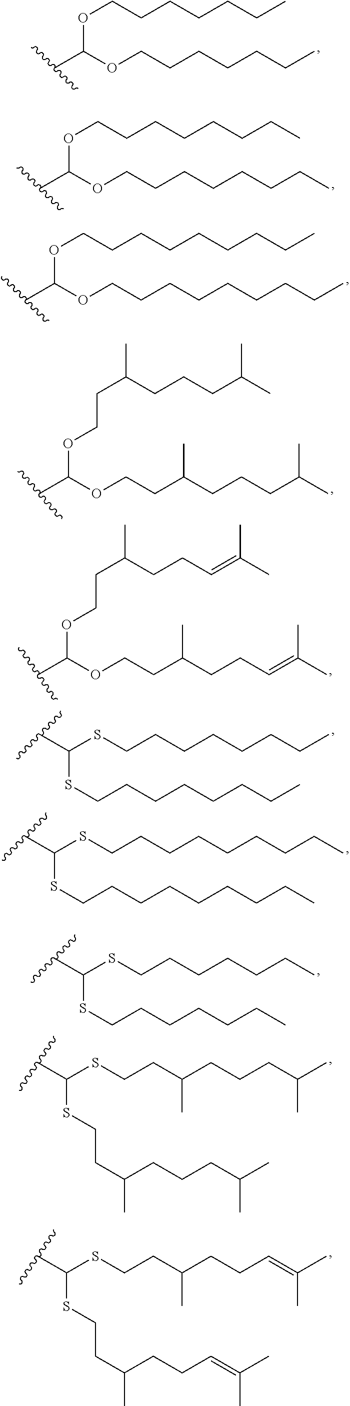

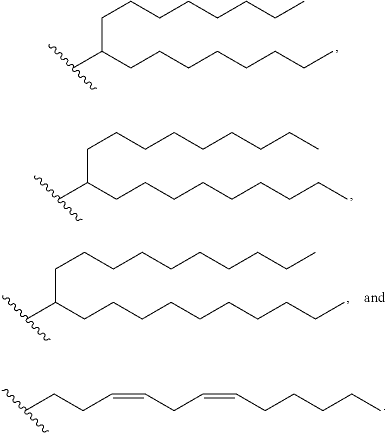 [SMILES: *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁵ is selected from

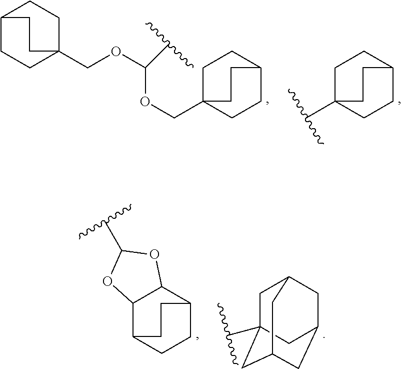

R⁶ and R⁷

As disclosed in Formula (CO), in certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkyl. In certain embodiments, R⁶ is optionally substituted C₁-C₁₄ branched alkyl. In certain embodiments, R⁶ is optionally substituted C₁-C₁₄ straight chain alkyl. In certain embodiments, R⁶ is optionally substituted C₁-C₁₄ alkenyl. In certain embodiments, R⁶ is optionally substituted C₁-C₁₄ branched alkenyl. In certain embodiments, R⁶ is optionally substituted C₁-C₁₄ straight chain alkenyl. In certain embodiments, R⁶ is optionally substituted C₆-C₁₀ alkyl. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

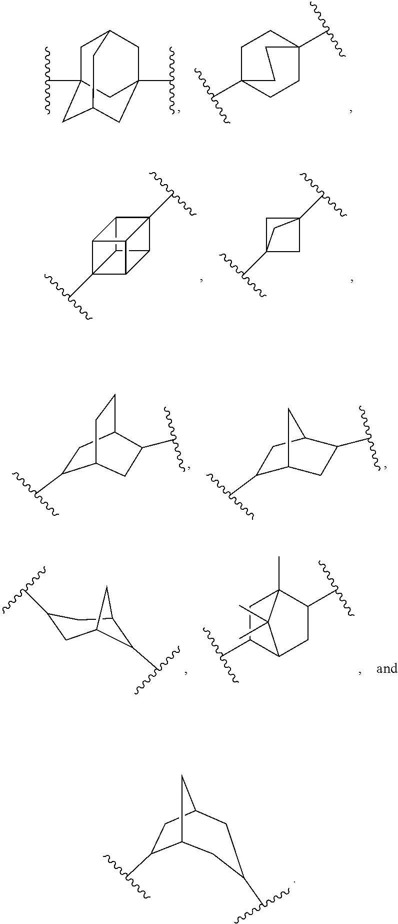

In certain embodiments, R⁷ is optionally substituted C₁-C₁₄ aliphatic. In certain embodiments, R⁷ is optionally substituted C₁-C₁₄ alkyl. In certain embodiments, R⁷ is optionally substituted C₁-C₁₄ branched alkyl. In certain embodiments, R⁷ is optionally substituted C₁-C₁₄ straight chain alkyl. In certain embodiments, R⁷ is optionally substituted C₁-C₁₄ alkenyl. In certain embodiments, R⁷ is optionally substituted C₁-C₁₄ branched alkenyl. In certain embodiments, R⁷ is optionally substituted C₁-C₁₄ straight chain alkenyl. In certain embodiments, R⁷ is optionally substituted C₆-C₁₀ alkyl. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

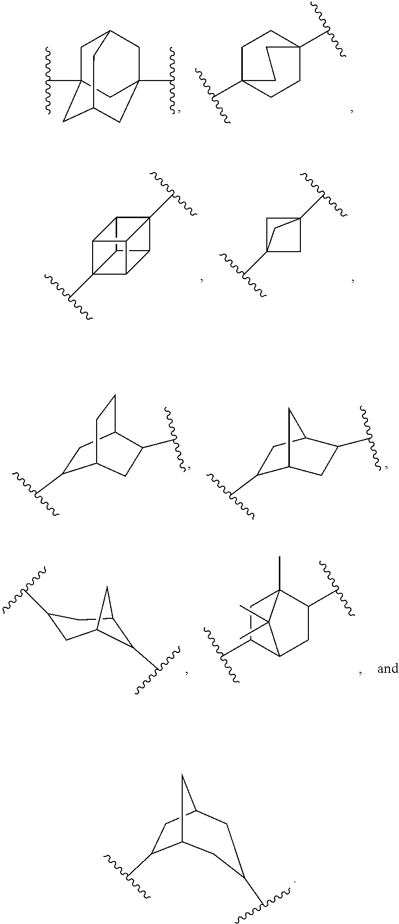

In certain embodiments, R⁶ and R⁷ are selected from

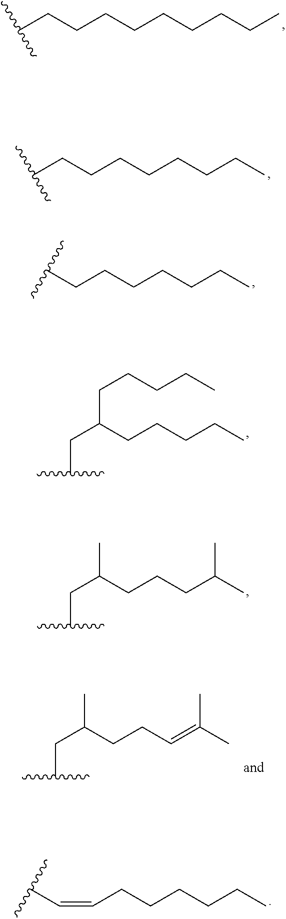

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

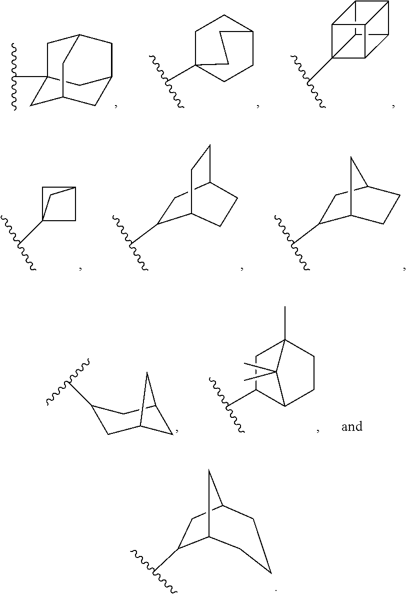

In certain embodiments, the substituted bridged bicyclic or multicyclic C₅-C₁₂ cycloalkylenyl is a structure selected from

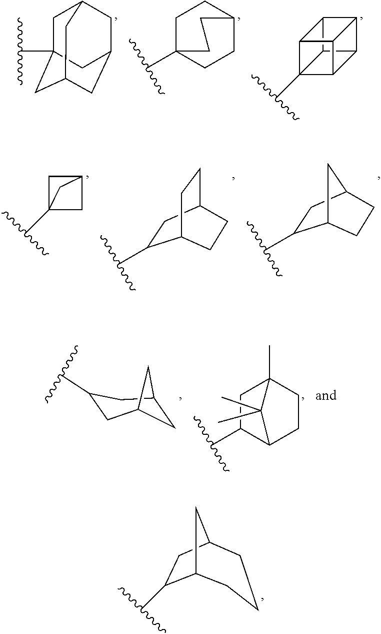

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

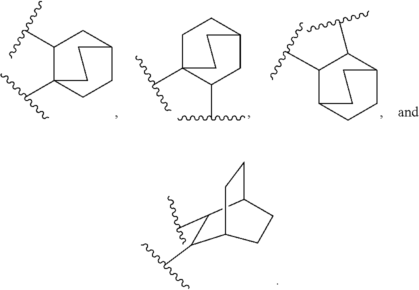

R⁸ and R⁹

As disclosed in Formula (CO), in certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R¹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂cycloalkylenyl is selected from:

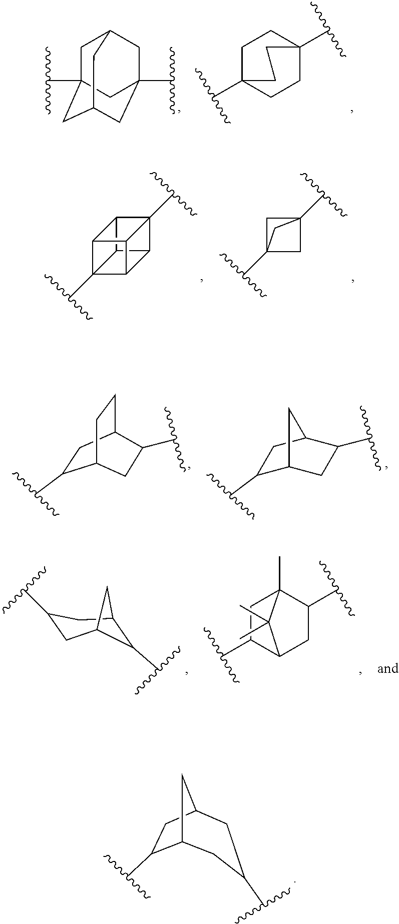

In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

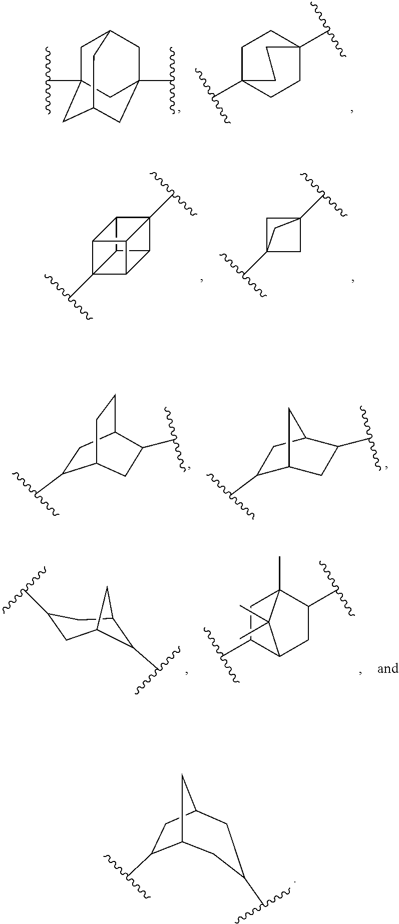

In certain embodiments, R⁸ and R⁹ are selected from

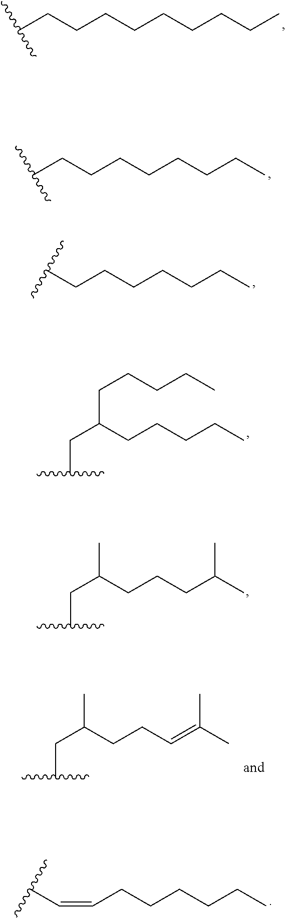

In some embodiments, R⁸ and R⁹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R⁸ and R⁹ are each independently selected from an optionally substituted bridged bicyclic C₅-C₁₂ cycloalkylenyl. In certain embodiments, R¹ is an optionally substituted bridged multicyclic C₅-C₁₂ cycloalkylenyl. In certain embodiments, R⁹ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

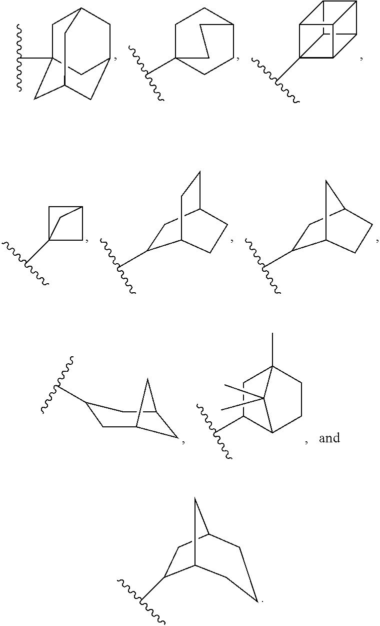

In certain embodiments, the substituted bridged bicyclic or multicyclic C₅-C₁₂ cycloalkylenyl is a structure selected from

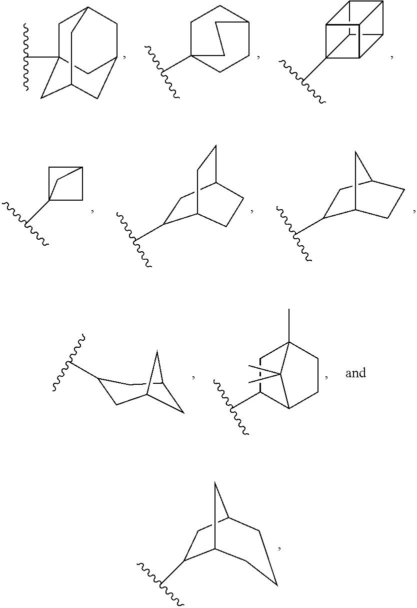

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

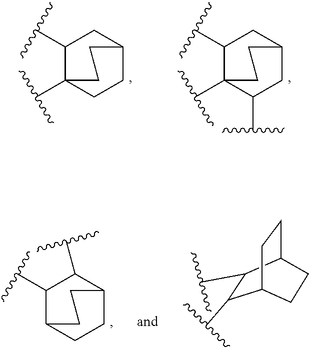 [SMILES: *C1CC2CCC1(*)CC2 ; *C1CC2CCC1(*)C(*)C2 ; *C1C2CCC(CC2)C1* ; *C1C2CCC(CC2)C1*]

In some embodiments, Lipids of the Present Disclosure are selected from any lipid in Table (I-I) below or a pharmaceutically acceptable salt thereof:

TABLE (I-I)
Non-Limiting Examples of Ionizable Lipids of the Present Disclosure

| Cmpd | Structure |
|---|---|
| CO-1 | 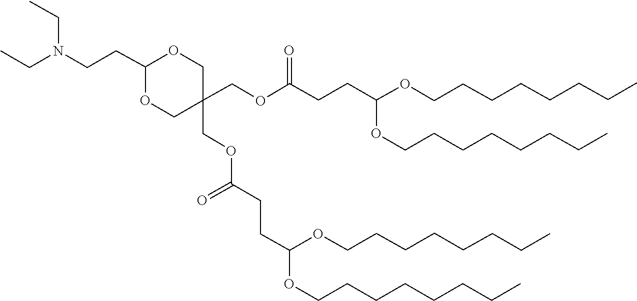 [SMILES: CCCCCCCCOC(CCC(=O)OCC1(COC(=O)CCC(OCCCCCCCC)OCCCCCCCC)COC(CCN(CC)CC)OC1)OCCCCCCCC] |
| CO-2 | 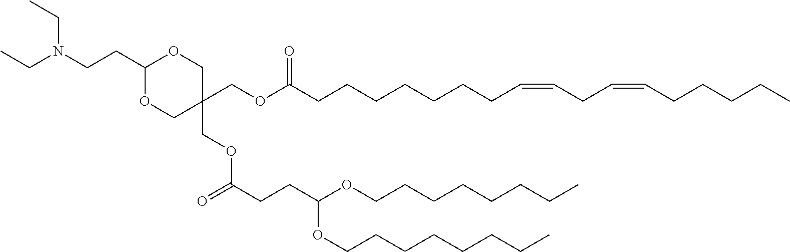 [SMILES: CCCCC/C=C\C/C=C\CCCCCCCC(=O)OCC1(COC(=O)CCC(OCCCCCCCC)OCCCCCCCC)COC(CCN(CC)CC)OC1] |
| CO-3 | 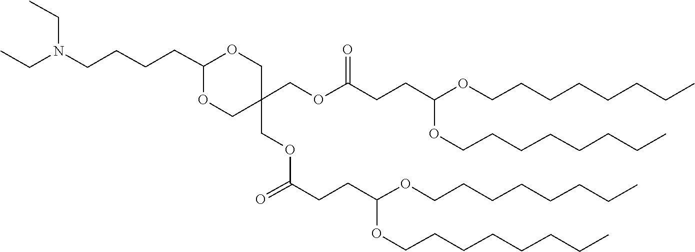 [SMILES: CCCCCCCCOC(CCC(=O)OCC1(COC(=O)CCC(OCCCCCCCC)OCCCCCCCC)COC(CCCCN(CC)CC)OC1)OCCCCCCCC] |
| CO-4 | 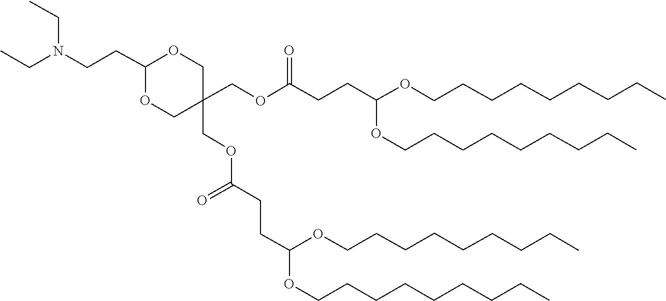 [SMILES: CCCCCCCCCOC(CCC(=O)OCC1(COC(=O)CCC(OCCCCCCCCC)OCCCCCCCCC)COC(CCN(CC)CC)OC1)OCCCCCCCCC] |
| CO-5 | 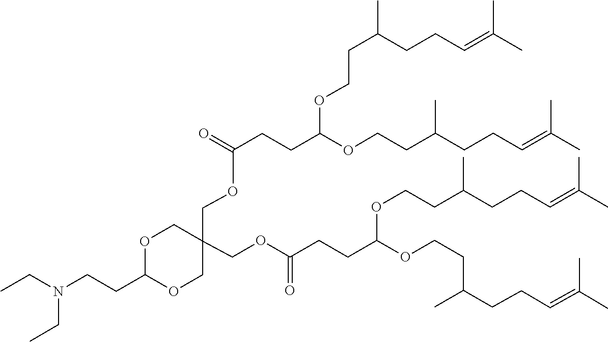 [SMILES: CCN(CC)CCC1OCC(COC(=O)CCC(OCCC(C)CCC=C(C)C)OCCC(C)CCC=C(C)C)(COC(=O)CCC(OCCC(C)CCC=C(C)C)OCCC(C)CCC=C(C)C)CO1] |
| CO-6 | 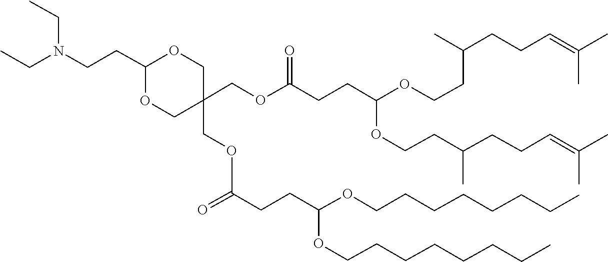 [SMILES: CCCCCCCCOC(CCC(=O)OCC1(COC(=O)CCC(OCCC(C)CCC=C(C)C)OCCC(C)CCC=C(C)C)COC(CCN(CC)CC)OC1)OCCCCCCCC] |
| CO-7 | 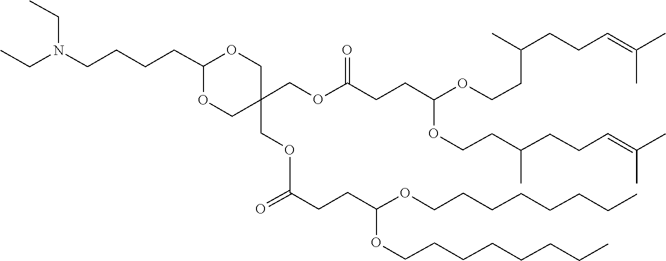 [SMILES: CCCCCCCCOC(CCC(=O)OCC1(COC(=O)CCC(OCCC(C)CCC=C(C)C)OCCC(C)CCC=C(C)C)COC(CCCCN(CC)CC)OC1)OCCCCCCCC] |
| CO-8 | 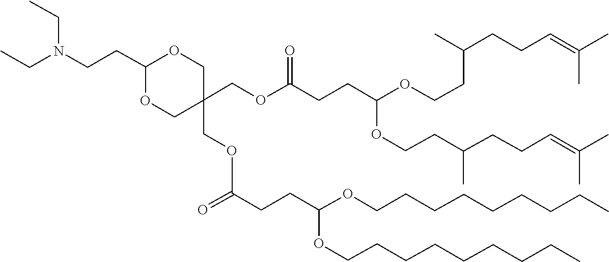 [SMILES: CCCCCCCCCOC(CCC(=O)OCC1(COC(=O)CCC(OCCC(C)CCC=C(C)C)OCCC(C)CCC=C(C)C)COC(CCN(CC)CC)OC1)OCCCCCCCCC] |
| CO-9 | 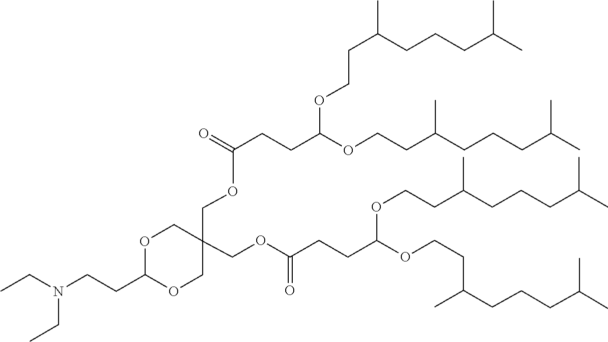 [SMILES: CCN(CC)CCC1OCC(COC(=O)CCC(OCCC(C)CCCC(C)C)OCCC(C)CCCC(C)C)(COC(=O)CCC(OCCC(C)CCCC(C)C)OCCC(C)CCCC(C)C)CO1] |
| CO-10 | 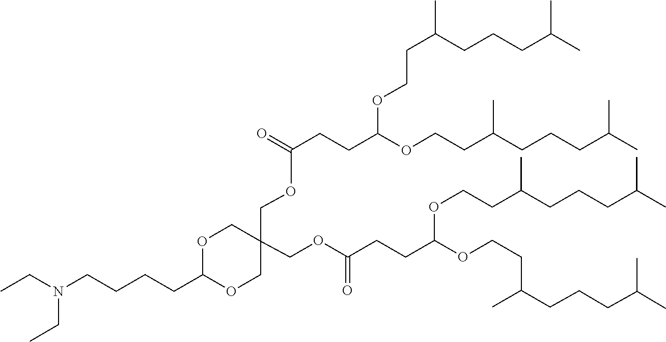 [SMILES: CCN(CC)CCCCC1OCC(COC(=O)CCC(OCCC(C)CCCC(C)C)OCCC(C)CCCC(C)C)(COC(=O)CCC(OCCC(C)CCCC(C)C)OCCC(C)CCCC(C)C)CO1] |
| CO-11 | 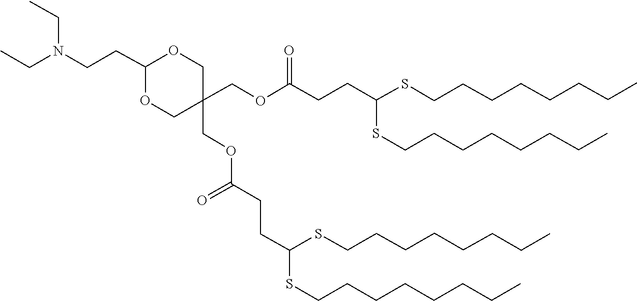 [SMILES: CCCCCCCCSC(CCC(=O)OCC1(COC(=O)CCC(SCCCCCCCC)SCCCCCCCC)COC(CCN(CC)CC)OC1)SCCCCCCCC] |

### Series “CC”

Described below are a number of exemplary ionizable lipids of the present disclosure. In certain embodiments, the ionizable lipid is one selected from those disclosed in PCT Application PCT/US2024/019990, which is incorporated by reference herein, in its entirety.

The present disclosure provides compound of Formula (CC)

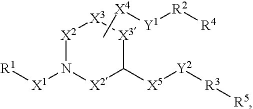

or a pharmaceutically acceptable salt thereof, wherein:
   R¹ is selected from the group consisting of —OH, —OAc, —NR₂,

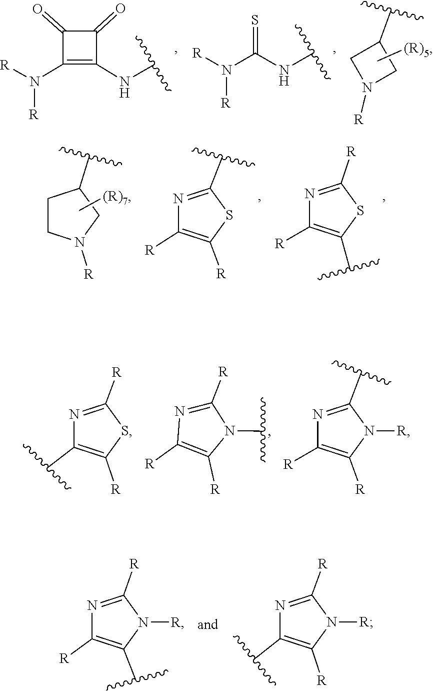

each R is independently —H or C₁-C₆ aliphatic;
X¹ is optionally substituted C₂-C₆ aliphatic, wherein one or more methylene linkages are each optionally and independently replaced with —O—, —NH—, —S—, —SS—, —C(O)—, —OC(O)O—, —OC(O)—, —NHC(O)— or —C(O)O—;
X² is selected from the group consisting of a bond, —CH₂— and —CH₂CH₂—;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₁-C₁₀ aliphatic;
Y¹ and Y² are independently selected from the group consisting of

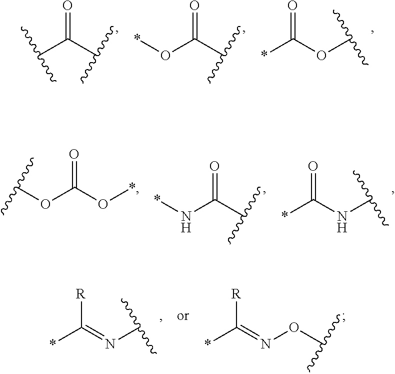

wherein the bond marked with an “*” is attached to X⁴ or X⁵;
R² is optionally substituted C₁-C₆ aliphatic;
R³ is optionally substituted C₁-C₆ aliphatic;
R⁴ is —CH(OR⁶)(OR⁷); —CH(SR⁶)(SR⁷); —CH(SR⁸)(SR⁹); —CH(R⁶)(R⁷); —R¹⁰; or optionally substituted C₁-C₁₄ aliphatic-R¹⁰ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
   R⁵ is —CH(OR⁸)(OR⁹); —CH(SR⁸)(SR⁹); —CH(R⁸)(R⁹); optionally substituted C₁-C₁₄ aliphatic, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; —R¹¹; or optionally substituted C₁-C₁₄ aliphatic-R¹¹, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
   R⁶ and R⁷ are each independently —R¹⁰; optionally substituted —C₁-C₁₄ aliphatic-R¹⁰; wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
   R⁸ and R⁹ are each independently —R¹¹; optionally substituted —C₁-C₁₄ aliphatic wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; or optionally substituted —C₁-C₁₄ aliphatic-R¹¹ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; and
each R¹⁰ and R¹¹ is independently an optionally substituted bridged bicyclic or multicyclic C₅-C₁₂ cycloalkylenyl, or two R¹⁰ or two R¹¹ taken together form an optionally substituted bridged bicyclic or multicyclic C₅-C₁₂ cycloalkylenyl; or
   each R¹⁰ and R¹¹ is independently an optionally substituted cyclic, bicyclic, bridged bicyclic, multicyclic or bridged multicyclic C₄-C₁₄ cycloalkyl or optionally substituted cyclic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

In certain embodiments, the compound of Formula (CC) is a compound of any one of the Formulae below:

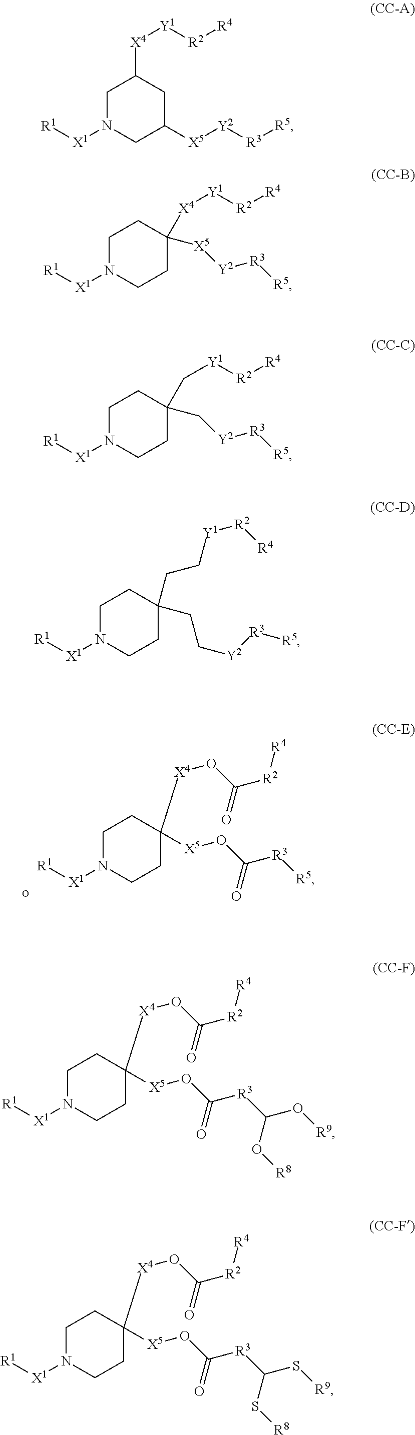

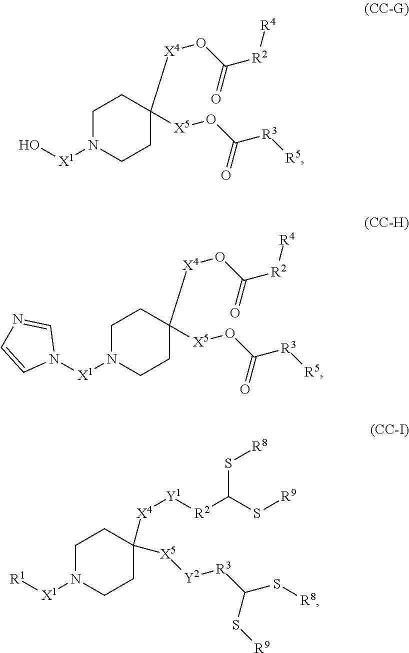

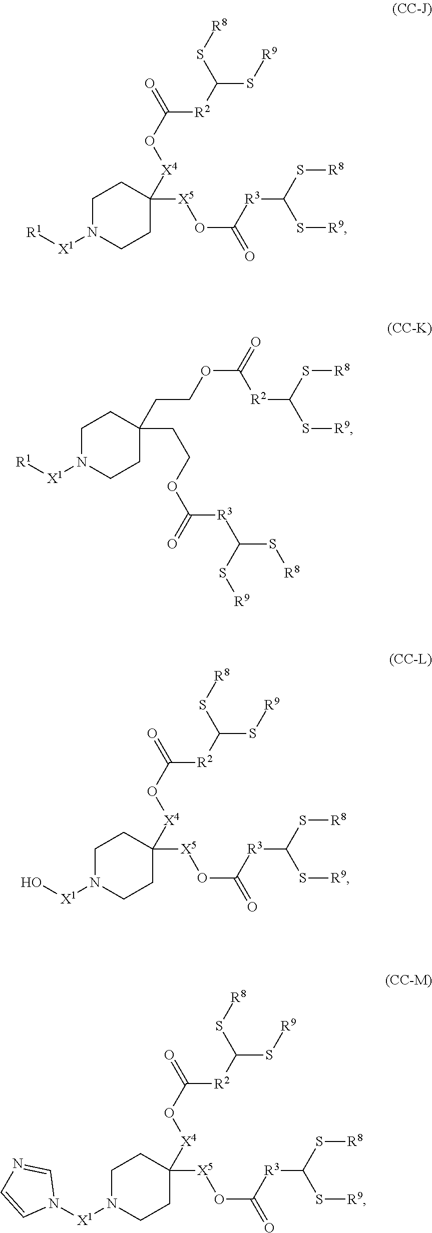

or a pharmaceutically acceptable salt thereof, wherein R¹, R, X¹, X⁴, X⁵, Y¹, Y², R², R³, R⁴, R⁵, R⁶, R⁷, R⁸, R⁹, R¹⁰, R¹¹, are as described in Formula (CC) or (CC′) or as otherwise described in any embodiments below.

### R¹

As disclosed in Formula (CC), in certain embodiments, R¹ is selected from the group consisting of —OH, —OAc, —NR₂,

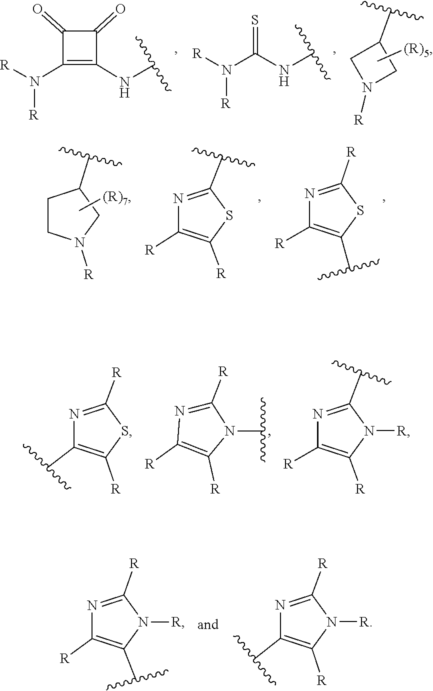

In certain embodiments, R¹ is —OH. In certain embodiments, R¹ is —OAc. In certain embodiments, R1 is —NR₂. In certain embodiments, R¹ is

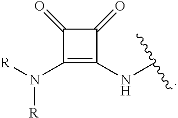

In certain embodiments, R¹ is

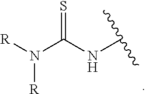

In certain embodiments, R¹ is

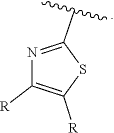

In certain embodiments, R¹ is

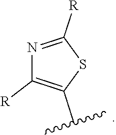

In certain embodiments, R¹ is

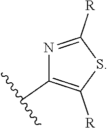

In certain embodiments, R¹ is

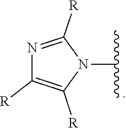

In certain embodiments, R¹ is

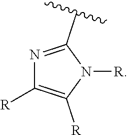

In certain embodiments, R¹ is

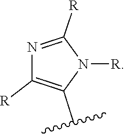

In certain embodiments, R¹ is

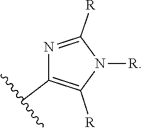

In certain embodiments, R¹ is

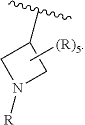

In certain embodiments, R¹ is

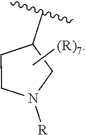

In certain embodiments, R¹ is —N(Et)₂. In certain embodiments, R¹ is —N(Me)₂. In certain embodiments, R¹ is —NH₂. In certain embodiments, R¹ is —N(nPr)₂. In certain embodiments, R¹ is —N(iPr)₂. In certain embodiments, R¹ is —N(Me)(Et). In certain embodiments, R¹ is OH. In certain embodiments, R¹ is

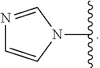 [SMILES: *n1ccnc1]

### X¹

As disclosed in Formula (CC), in certain embodiments, X¹ is optionally substituted C₂-C₆ aliphatic, wherein one or more methylene linkages are each optionally and independently replaced with —O—, —NH—, —S—, —SS—, —C(O)—, —OC(O)O—, —NHC(O)— or —C(O)O—. In certain embodiments, X¹ is optionally substituted C₂-C₆ aliphatic. In certain embodiments, X¹ is optionally substituted C₂-C₆ alkylene. In certain embodiments, X¹ is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### X²

As disclosed in Formula (CC), in certain embodiments, X² is selected from the group consisting of a bond, —CH₂— and —CH₂CH₂—. In certain embodiments, X² is a bond. In certain embodiments, X² is —CH₂—. In certain embodiments, X² is —CH₂CH₂—.

### X^{2′}

As disclosed in Formula (CC), in certain embodiments, X^{2′} is selected from the group consisting of a bond, —CH₂— and —CH₂CH₂—. In certain embodiments, X^{2′} is a bond. In certain embodiments, X^{2′} is —CH₂—. In certain embodiments, X^{2′} is —CH₂CH₂—.

### X³

As disclosed in Formula (CC), in certain embodiments, X³ is selected from the group consisting of a bond, —CH₂— and —CH₂CH₂—. In certain embodiments, X³ is a bond. In certain embodiments, X³ is —CH₂—. In certain embodiments, X³ is —CH₂CH₂—.

### X³

As disclosed in Formula (CC), in certain embodiments, X^{3′} is selected from the group consisting of a bond, —CH₂— and —CH₂CH₂—. In certain embodiments, X^{3′} is a bond. In certain embodiments, X^{3′} is —CH₂—. In certain embodiments, X^{3′} is —CH₂CH₂—.

In certain embodiments, each of X², X^{2′}, X³ and X^{3′} are each —CH₂—. In certain embodiments, both X² and X³ are each —CH₂—; X^{3′} is a bond, and X^{2′} is —CH₂CH₂—.

X⁴ and X⁵

As disclosed in Formula (CC), in certain embodiments, X⁴ and X⁵ are each independently optionally substituted C₁-C₁₀ aliphatic. In certain embodiments, X⁴ and X⁵ are the same. In certain embodiments, X⁴ and X⁵ are different.

In certain embodiments, X⁴ is an optionally substituted C₁-C₁₀ alkylene. In certain embodiments, X⁴ is an optionally substituted C₁-C₁₀ alkenylene. In certain embodiments, X⁴ is an optionally substituted C₁-C₆ alkylene. In certain embodiments, X⁴ is an optionally substituted C₁-C₆ alkenylene. In certain embodiments, X⁴ is —(CH₂)—. In certain embodiments, X⁴ is —(CH₂)₂—. In certain embodiments, X⁴ is —(CH₂)₃—. In certain embodiments, X⁴ is —(CH₂)₄—. In certain embodiments, X⁴ is —(CH₂)₅—. In certain embodiments, X⁴ is —(CH₂)₆—.

In certain embodiments, X⁵ is an optionally substituted C₁-C₁₀ alkylene. In certain embodiments, X⁵ is an optionally substituted C₁-C₁₀ alkenylene. In certain embodiments, X⁵ is an optionally substituted C₁-C₆ alkylene. In certain embodiments, X⁵ is an optionally substituted C₁-C₆ alkenylene. In certain embodiments, X⁵ is —(CH₂)—. In certain embodiments, X⁵ is —(CH₂)₂—. In certain embodiments, X⁵ is —(CH₂)₃—. In certain embodiments, X⁵ is —(CH₂)₄—. In certain embodiments, X⁵ is —(CH₂)₅—. In certain embodiments, X⁵ is —(CH₂)₆—.

In certain embodiments, X⁴ and X⁵ are both —(CH₂)—. In certain embodiments, X⁴ and X⁵ are both —(CH₂)₂—.

Y¹ and Y²

As disclosed in Formula (CC), in certain embodiments, Y¹ and Y² are each independently

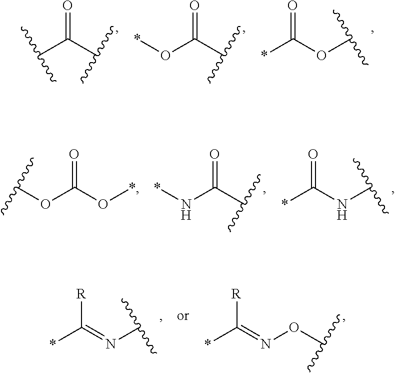

wherein the bond marked with an “*” is attached to X⁴ or X⁵. In certain embodiments, Y¹ and Y² are the same. In certain embodiments, Y¹ and Y² are different.

In certain embodiments, Y¹ is

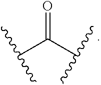 [SMILES: *C(*)=O]

In certain embodiments, Y¹ is

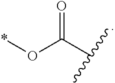 [SMILES: *OC(*)=O]

In certain embodiments, Y¹ is

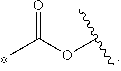 [SMILES: *OC(C)=O]

In certain embodiments, Y¹

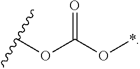

In certain embodiments, Y¹ is

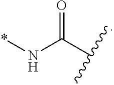 [SMILES: *NC(*)=O]

In certain embodiments, Y¹ is

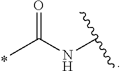

In certain embodiments, Y¹ is

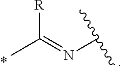

In certain embodiments, Y¹ is

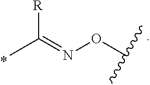

In certain embodiments, Y² is

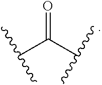 [SMILES: *C(*)=O]

In certain embodiments, Y² is

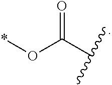 [SMILES: *OC(*)=O]

In certain embodiments, Y² is

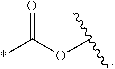 [SMILES: *OC(*)=O]

In certain embodiments, Y² is

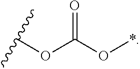

In certain embodiments, Y² is

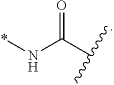 [SMILES: *NC(*)=O]

In certain embodiments, Y² is

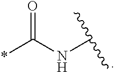 [SMILES: *NC(*)=O]

In certain embodiments, Y² is

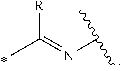

In certain embodiments, Y² is

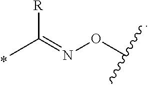

In certain embodiments, Y¹ and Y² are both

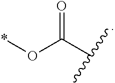 [SMILES: *OC(*)=O]

In certain embodiments, Y¹ and Y² are both

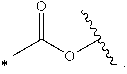 [SMILES: *OC(C)=O]

### R²

As disclosed in Formula (CC), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (CC), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different. In certain embodiments, R² and R³ are both —(CH₂)₂—.

### R⁴

As disclosed in Formula (CC), in certain embodiments, R⁴ is —CH(OR⁶)(OR⁷); —CH(SR⁶)(SR⁷); —CH(SR⁸)(SR⁹); —CH(R⁶)(R⁷); —R¹⁰; or optionally substituted C₁-C₁₄ aliphatic-R¹⁰ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic-R¹⁰, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic-R¹⁰. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷). In certain embodiments, R⁴ is —CH(SR⁸)(SR⁹). In certain embodiments, R⁴ is R¹⁰.

In certain embodiments, R⁴ is selected from

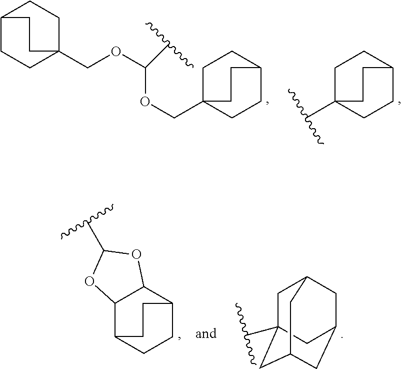 [SMILES: *C(OCC12CCC(CC1)CC2)OCC12CCC(CC1)CC2 ; *C12CCC(CC1)CC2 ; *C1OC2C3CCC(CC3)C2O1 ; *C12CC3CC(CC(C3)C1)C2]

In certain embodiments, R⁴ is selected from

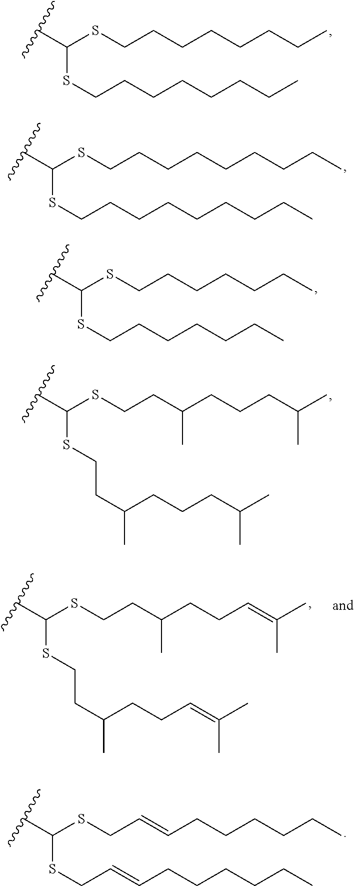 [SMILES: *C(SCCCCCCCC)SCCCCCCCC ; *C(SCCCCCCCCC)SCCCCCCCCC ; *C(SCCCCCCC)SCCCCCCC ; *C(SCCC(C)CCCC(C)C)SCCC(C)CCCC(C)C ; *C(SCCC(C)CCC=C(C)C)SCCC(C)CCC=C(C)C ; *C(SC/C=C/CCCCCC)SC/C=C/CCCCCC]

As disclosed in Formula (CC), in certain embodiments, R⁵ is —CH(OR⁸)(OR⁹); —CH(SR⁸)(SR⁹); —CH(R⁸)(R⁹); optionally substituted C₁-C₁₄ aliphatic, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; —R¹¹; or optionally substituted C₁-C₁₄ aliphatic-R¹¹, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹). In certain embodiments, R⁵ is R¹¹.

In certain embodiments, R⁴ and R⁵ are the same. In certain embodiments, R⁴ and R⁵ are different.

In certain embodiments, R⁵ is selected from

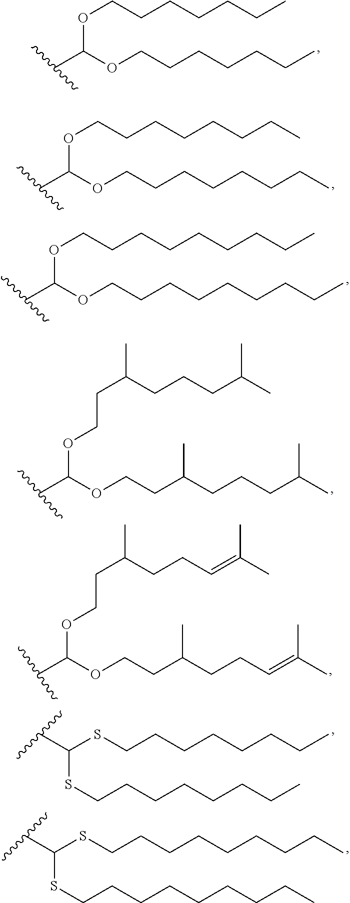

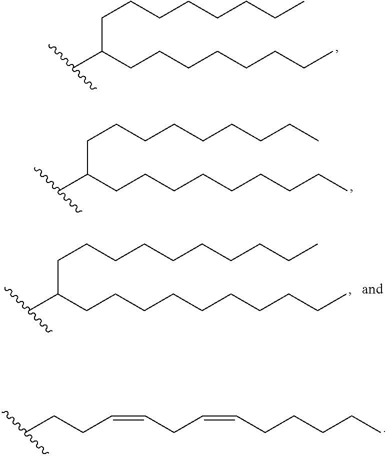 [SMILES: *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁵ is selected from

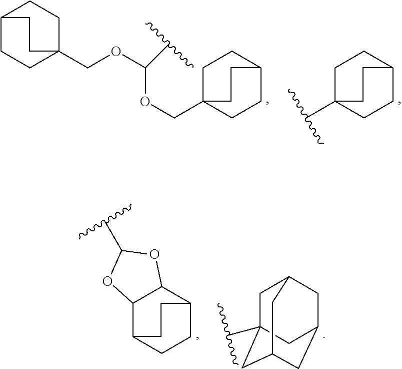

R⁶ and R⁷

As disclosed in Formula (CC), in certain embodiments, R⁶ and R⁷ are each independently —R¹⁰; optionally substituted —C₁-C₁₄ aliphatic-R¹⁰; wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is R¹⁰. In certain embodiments, R⁶ is optionally substituted C₁-C₁₄ aliphatic-R¹⁰. In certain embodiments, R⁶ is optionally substituted C₁-C₁₄ alkyl-R¹⁰. In certain embodiments, R⁶ is optionally substituted C₁-C₁₄ branched alkyl-R¹⁰. In certain embodiments, R⁶ is optionally substituted C₁-C₁₄ straight chain alkyl-R¹⁰. In certain embodiments, R⁶ is optionally substituted C₁-C₁₄ alkenyl-R¹⁰. In certain embodiments, R⁶ is optionally substituted C₁-C₁₄ branched alkenyl-R¹⁰. In certain embodiments, R⁶ is optionally substituted C₁-C₁₄ straight chain alkenyl-R¹⁰. In certain embodiments, R⁶ is optionally substituted C₁-C₅ alkyl-R¹⁰. In certain embodiments, R⁶ is optionally substituted —(CH₂)—R¹⁰. In certain embodiments, R⁶ is optionally substituted —(CH₂)₂—R¹⁰. In certain embodiments, R⁶ is optionally substituted —(CH₂)₃—R¹⁰. In certain embodiments, R⁶ is optionally substituted —(CH₂)₄-R¹⁰. In certain embodiments, R⁶ is optionally substituted —(CH₂)₅-R¹⁰.

In certain embodiments, R⁷ is R¹⁰. In certain embodiments, R⁷ is optionally substituted C₁-C₁₄ aliphatic-R¹⁰. In certain embodiments, R⁷ is optionally substituted C₁-C₁₄ alkyl-R¹⁰. In certain embodiments, R⁷ is optionally substituted C₁-C₁₄ branched alkyl-R¹⁰. In certain embodiments, R⁷ is optionally substituted C₁-C₁₄ straight chain alkyl-R¹⁰. In certain embodiments, R⁷ is optionally substituted C₁-C₁₄ alkenyl-R¹⁰. In certain embodiments, R⁷ is optionally substituted C₁-C₁₄ branched alkenyl-R¹⁰. In certain embodiments, R⁷ is optionally substituted C₁-C₁₄ straight chain alkenyl-R¹⁰. In certain embodiments, R⁷ is optionally substituted C₁-C₅ alkyl-R¹⁰. In certain embodiments, R⁷ is optionally substituted —(CH₂)—R¹⁰. In certain embodiments, R⁷ is optionally substituted —(CH₂)₂-R¹⁰. In certain embodiments, R⁷ is optionally substituted —(CH₂)₃-R¹⁰. In certain embodiments, R⁷ is optionally substituted —(CH₂)₄-R¹⁰. In certain embodiments, R⁷ is optionally substituted —(CH₂)₅—R¹⁰.

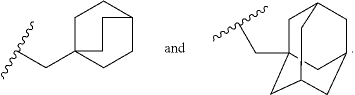 [SMILES: *CC12CCC(CC1)CC2 ; *CC12CC3CC(CC(C3)C1)C2]

In certain embodiments, R⁶ and R⁷ are selected from and

R⁸ and R⁹

As disclosed in Formula (CC), in certain embodiments, R⁸ and R⁹ are each independently R¹; optionally substituted —C₁-C₁₄ aliphatic wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; or optionally substituted —C₁-C₁₄ aliphatic-R¹ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is R¹¹. In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, R⁸ is optionally substituted C₁-C₁₄ aliphatic-R¹¹. In certain embodiments, R⁸ is optionally substituted C₁-C₁₄ alkylene-R¹¹. In certain embodiments, R⁸ is optionally substituted C₁-C₁₄ branched alkylene-R¹¹. In certain embodiments, R⁸ is optionally substituted C₁-C₁₄ straight chain alkylene-R¹¹. In certain embodiments, R⁸ is optionally substituted C₁-C₁₄ alkenylene-R¹¹. In certain embodiments, R⁸ is optionally substituted C₁-C₁₄ branched alkenylene-R¹¹. In certain embodiments, R⁸ is optionally substituted C₁-C₁₄ straight chain alkenylene-R¹¹. In certain embodiments, R⁸ is optionally substituted C₁-C₈ alkylene-R¹¹. In certain embodiments, R⁸ is optionally substituted —(CH₂)—R¹¹. In certain embodiments, R⁸ is optionally substituted —(CH₂)₂-R¹¹. In certain embodiments, R⁸ is optionally substituted —(CH₂)₃-R¹¹. In certain embodiments, R⁸ is optionally substituted —(CH₂)₄-R¹¹. In certain embodiments, R⁸ is optionally substituted —(CH₂)₅—R¹¹.

In certain embodiments, R⁹ is R¹¹. In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, R⁹ is optionally substituted C₁-C₁₄ aliphatic-R¹¹. In certain embodiments, R⁹ is optionally substituted C₁-C₁₄ alkylene-R¹¹. In certain embodiments, R⁹ is optionally substituted C₁-C₁₄ branched alkylene-R¹¹. In certain embodiments, R⁹ is optionally substituted C₁-C₁₄ straight chain alkylene-R¹¹. In certain embodiments, R⁹ is optionally substituted C₁-C₁₄ alkenylene-R¹¹. In certain embodiments, R⁹ is optionally substituted C₁-C₁₄ branched alkenylene-R¹¹. In certain embodiments, R⁹ is optionally substituted C₁-C₁₄ straight chain alkenylene-R¹¹. In certain embodiments, R⁹ is optionally substituted C₁-C₅ alkylene-R¹¹. In certain embodiments, R⁹ is optionally substituted —(CH₂)—R¹¹. In certain embodiments, R⁹ is optionally substituted —(CH₂)₂-R¹¹. In certain embodiments, R⁹ is optionally substituted —(CH₂)₃—R¹¹. In certain embodiments, R⁹ is optionally substituted —(CH₂)₄—R¹¹. In certain embodiments, R⁹ is optionally substituted —(CH₂)₅-R¹¹.

In certain embodiments, R⁸ and R⁹ are selected from

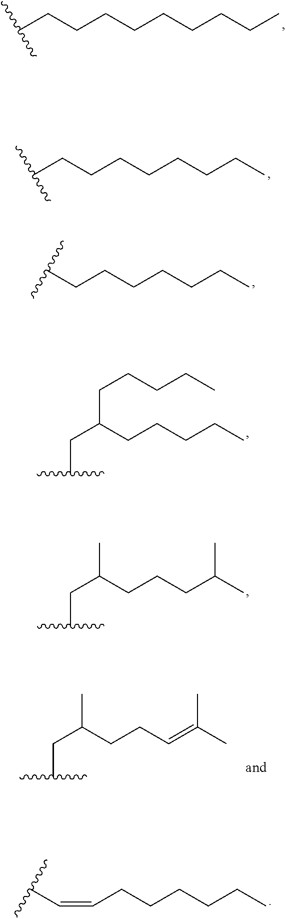

In certain embodiments, R⁸ and R⁹ are selected from

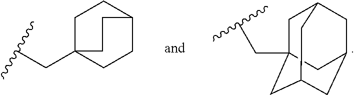 [SMILES: *CC12CCC(CC1)CC2 ; *CC12CC3CC(CC(C3)C1)C2]

R¹⁰ and R¹¹

As disclosed in Formula (CC), in certain embodiments, each R¹⁰ and R¹¹ are an optionally substituted bridged bicyclic or multicyclic C₅-C₁₂ cycloalkylenyl, or two R¹⁰ or two R¹¹ taken together form an optionally substituted bridged bicyclic or multicyclic C₅-C₁₂ cycloalkylenyl.

In certain embodiments, each R¹⁰ and R¹¹ are the same. In certain embodiments, each R¹⁰ and R¹¹ are different.

In some embodiments, each R¹⁰ and R¹¹ is independently an optionally substituted cyclic, bicyclic, bridged bicyclic, multicyclic or bridged multicyclic C₄-C₁₄ cycloalkyl or optionally substituted cyclic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R¹⁰ is an optionally substituted bridged bicyclic C₅-C₁₂ cycloalkylenyl. In certain embodiments, each R¹⁰ is an optionally substituted bridged multicyclic C₅-C₁₂ cycloalkylenyl. In certain embodiments, each R¹¹ is an optionally substituted bridged bicyclic C₅-C₁₂ cycloalkylenyl. In certain embodiments, each R¹¹ is an optionally substituted bridged multicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

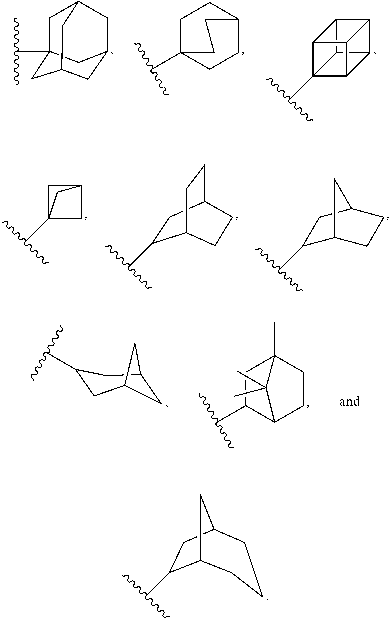

In certain embodiments, the substituted bridged bicyclic or multicyclic C₅-C₁₂ cycloalkylenyl is a structure selected from

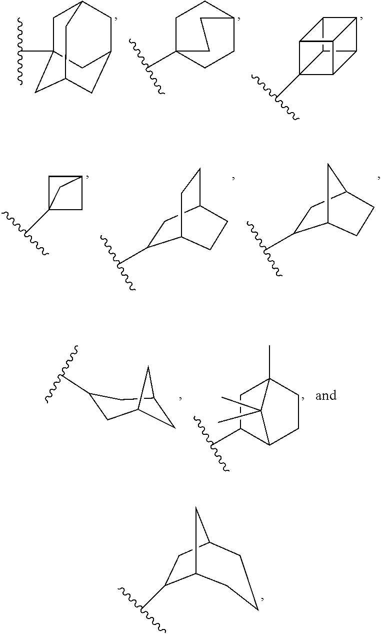

wherein one or more C—H bonds are substituted.

In certain embodiments, two R¹⁰ taken together form an optionally substituted bridged bicyclic or multicyclic C₅-C₁₂ cycloalkylenyl. In certain embodiments, two R¹¹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

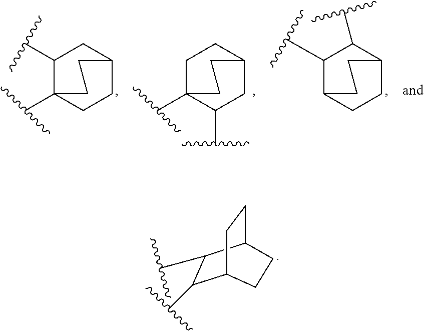 [SMILES: *C1CC2CCC1(*)CC2 ; *C1CC2CCC1(*)CC2 ; *C1C2CCC(CC2)C1* ; *C1C2CCC(CC2)C1*]

In some embodiments, Lipids of the Present Disclosure are selected from any lipid in Table (I-J) below or a pharmaceutically acceptable salt thereof:

TABLE (I-J)
Non-Limiting Examples of Ionizable Lipids of the Present Disclosure

| Cmpd | Structure |
|---|---|
| CC-1 | 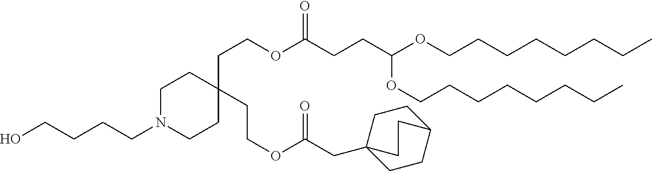 [SMILES: CCCCCCCCOC(CCC(=O)OCCC1(CCOC(=O)CC23CCC(CC2)CC3)CCN(CCCCO)CC1)OCCCCCCCC] |
| CC-2 | 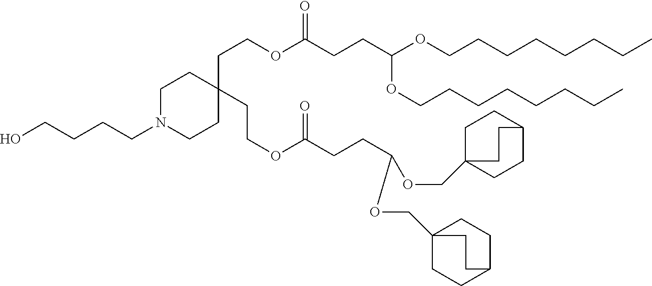 [SMILES: CCCCCCCCOC(CCC(=O)OCCC1(CCOC(=O)CCC(OCC23CCC(CC2)CC3)OCC23CCC(CC2)CC3)CCN(CCCCO)CC1)OCCCCCCCC] |
| CC-3 | 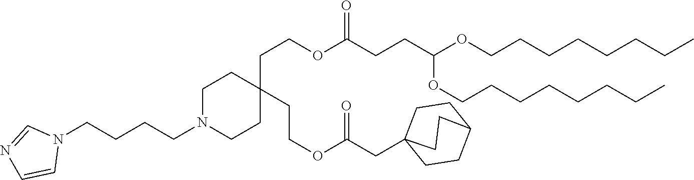 [SMILES: CCCCCCCCOC(CCC(=O)OCCC1(CCOC(=O)CC23CCC(CC2)CC3)CCN(CCCCn2ccnc2)CC1)OCCCCCCCC] |
| CC-4 | 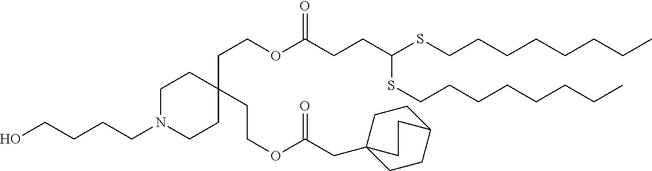 [SMILES: CCCCCCCCSC(CCC(=O)OCCC1(CCOC(=O)CC23CCC(CC2)CC3)CCN(CCCCO)CC1)SCCCCCCCC] |
| CC-5 | 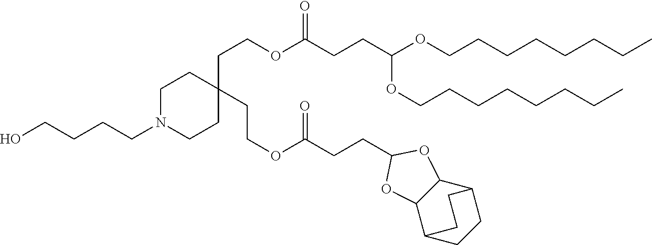 [SMILES: CCCCCCCCOC(CCC(=O)OCCC1(CCOC(=O)CCC2OC3C4CCC(CC4)C3O2)CCN(CCCCO)CC1)OCCCCCCCC] |
| CC-6 | 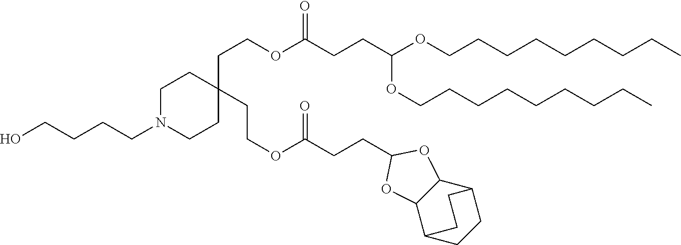 [SMILES: CCCCCCCCCOC(CCC(=O)OCCC1(CCOC(=O)CCC2OC3C4CCC(CC4)C3O2)CCN(CCCCO)CC1)OCCCCCCCCC] |
| CC-7 | 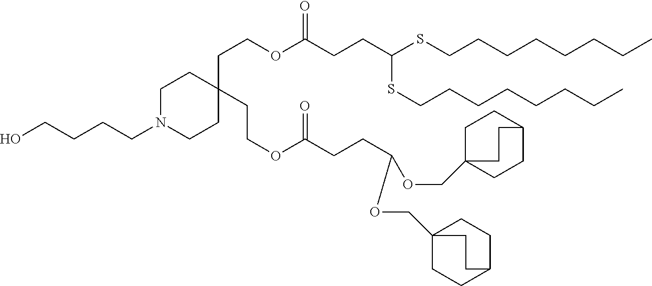 [SMILES: CCCCCCCCSC(CCC(=O)OCCC1(CCOC(=O)CCC(OCC23CCC(CC2)CC3)OCC23CCC(CC2)CC3)CCN(CCCCO)CC1)SCCCCCCCC] |
| CC-8 | 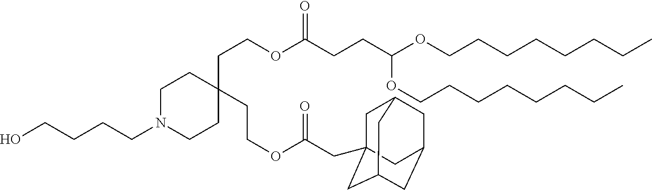 [SMILES: CCCCCCCCOC(CCC(=O)OCCC1(CCOC(=O)CC23CC4CC(CC(C4)C2)C3)CCN(CCCCO)CC1)OCCCCCCCC] |
| CC-9 | 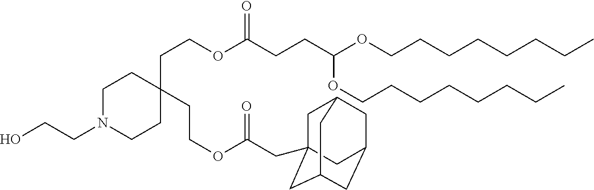 [SMILES: CCCCCCCCOC(CCC(=O)OCCC1(CCOC(=O)CC23CC4CC(CC(C4)C2)C3)CCN(CCO)CC1)OCCCCCCCC] |
| CC-10 | 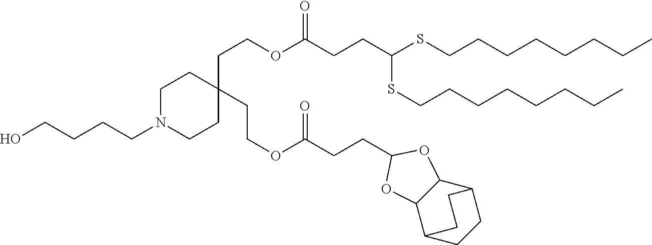 [SMILES: CCCCCCCCSC(CCC(=O)OCCC1(CCOC(=O)CCC2OC3C4CCC(CC4)C3O2)CCN(CCCCO)CC1)SCCCCCCCC] |
| CC-11 | 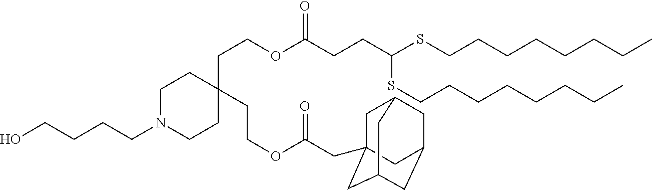 [SMILES: CCCCCCCCSC(CCC(=O)OCCC1(CCOC(=O)CC23CC4CC(CC(C4)C2)C3)CCN(CCCCO)CC1)SCCCCCCCC] |
| CC-12 | 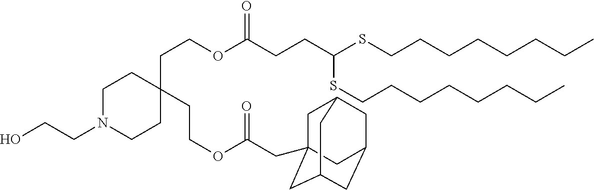 [SMILES: CCCCCCCCSC(CCC(=O)OCCC1(CCOC(=O)CC23CC4CC(CC(C4)C2)C3)CCN(CCO)CC1)SCCCCCCCC] |
| CC-13 | 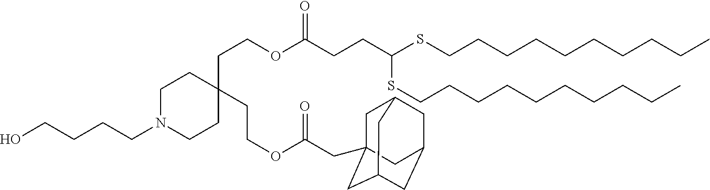 [SMILES: CCCCCCCCCCSC(CCC(=O)OCCC1(CCOC(=O)CC23CC4CC(CC(C4)C2)C3)CCN(CCCCO)CC1)SCCCCCCCCCC] |
| CC-14 | 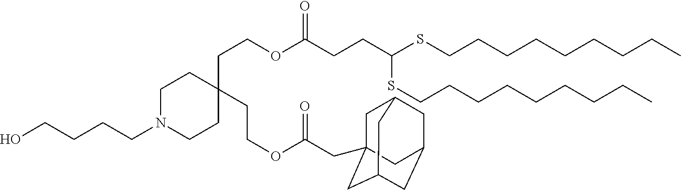 [SMILES: CCCCCCCCCSC(CCC(=O)OCCC1(CCOC(=O)CC23CC4CC(CC(C4)C2)C3)CCN(CCCCO)CC1)SCCCCCCCCC] |
| CC-15 | 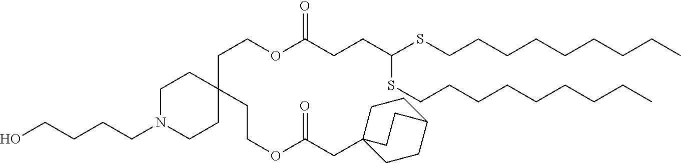 [SMILES: CCCCCCCCCSC(CCC(=O)OCCC1(CCOC(=O)CC23CCC(CC2)CC3)CCN(CCCCO)CC1)SCCCCCCCCC] |
| CC-16 | 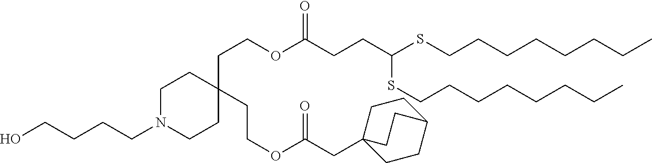 [SMILES: CCCCCCCCSC(CCC(=O)OCCC1(CCOC(=O)CC23CCC(CC2)CC3)CCN(CCCCO)CC1)SCCCCCCCC] |
| CC-17 | 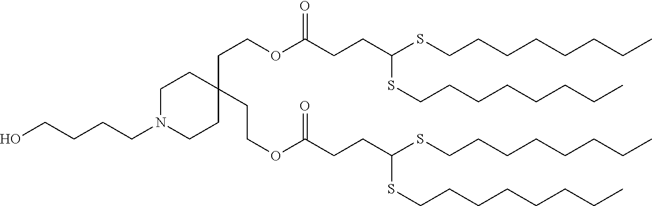 [SMILES: CCCCCCCCSC(CCC(=O)OCCC1(CCOC(=O)CCC(SCCCCCCCC)SCCCCCCCC)CCN(CCCCO)CC1)SCCCCCCCC] |
| CC-18 | 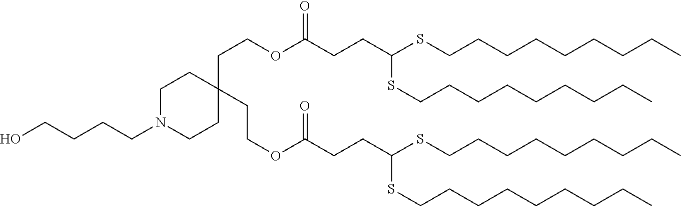 [SMILES: CCCCCCCCCSC(CCC(=O)OCCC1(CCOC(=O)CCC(SCCCCCCCCC)SCCCCCCCCC)CCN(CCCCO)CC1)SCCCCCCCCC] |
| CC-19 | 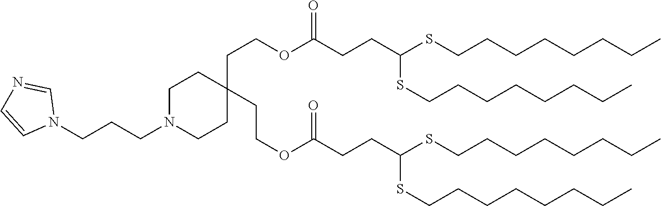 [SMILES: CCCCCCCCSC(CCC(=O)OCCC1(CCOC(=O)CCC(SCCCCCCCC)SCCCCCCCC)CCN(CCCn2ccnc2)CC1)SCCCCCCCC] |
| CC-20 | 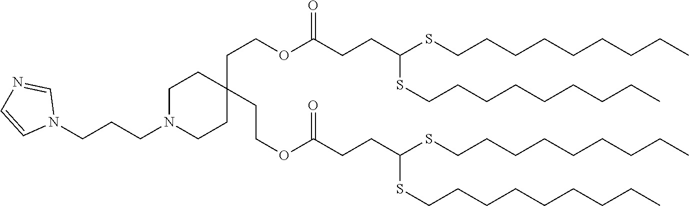 [SMILES: CCCCCCCCCSC(CCC(=O)OCCC1(CCOC(=O)CCC(SCCCCCCCCC)SCCCCCCCCC)CCN(CCCn2ccnc2)CC1)SCCCCCCCCC] |
| CC-21 | 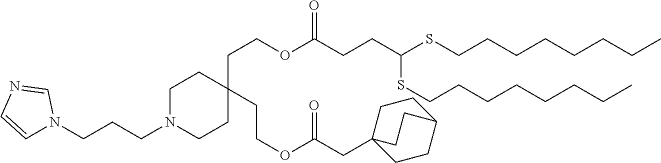 [SMILES: CCCCCCCCSC(CCC(=O)OCCC1(CCOC(=O)CC23CCC(CC2)CC3)CCN(CCCn2ccnc2)CC1)SCCCCCCCC] |
| CC-22 | 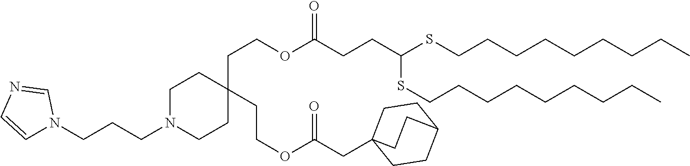 [SMILES: CCCCCCCCCSC(CCC(=O)OCCC1(CCOC(=O)CC23CCC(CC2)CC3)CCN(CCCn2ccnc2)CC1)SCCCCCCCCC] |
| CC-23 | 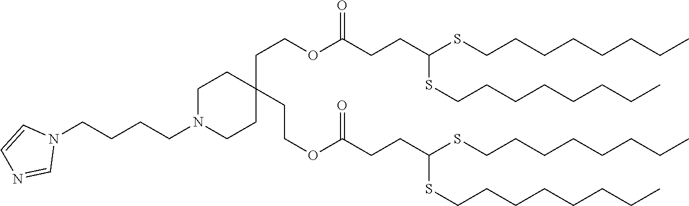 [SMILES: CCCCCCCCSC(CCC(=O)OCCC1(CCOC(=O)CCC(SCCCCCCCC)SCCCCCCCC)CCN(CCCCn2ccnc2)CC1)SCCCCCCCC] |
| CC-24 | 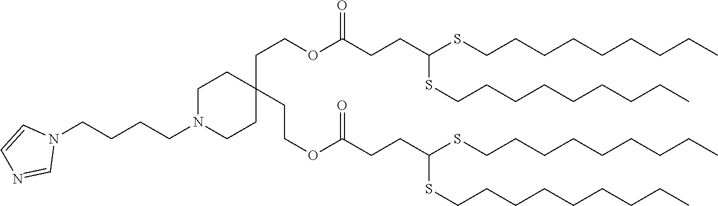 [SMILES: CCCCCCCCCSC(CCC(=O)OCCC1(CCOC(=O)CCC(SCCCCCCCCC)SCCCCCCCCC)CCN(CCCCn2ccnc2)CC1)SCCCCCCCCC] |
| CC-25 | 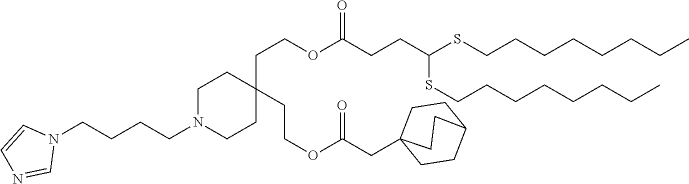 [SMILES: CCCCCCCCSC(CCC(=O)OCCC1(CCOC(=O)CC23CCC(CC2)CC3)CCN(CCCCn2ccnc2)CC1)SCCCCCCCC] |
| CC-26 | 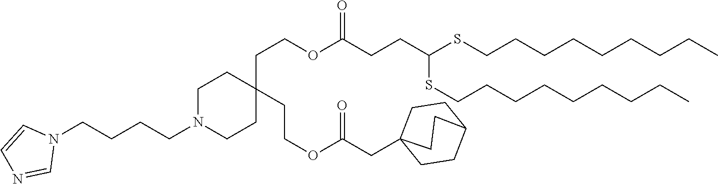 [SMILES: CCCCCCCCCSC(CCC(=O)OCCC1(CCOC(=O)CC23CCC(CC2)CC3)CCN(CCCCn2ccnc2)CC1)SCCCCCCCCC] |
| CC-27 | 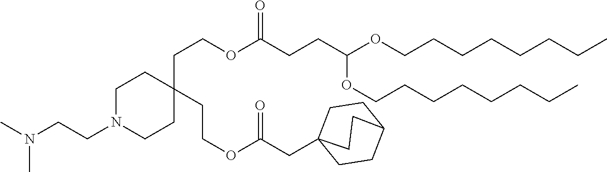 [SMILES: CCCCCCCCOC(CCC(=O)OCCC1(CCOC(=O)CC23CCC(CC2)CC3)CCN(CCN(C)C)CC1)OCCCCCCCC] |
| CC- 100 | 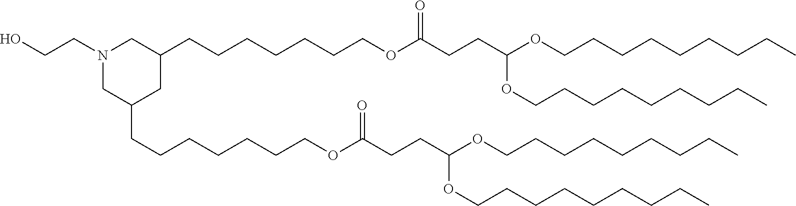 [SMILES: CCCCCCCCCOC(CCC(=O)OCCCCCCCC1CC(CCCCCCCOC(=O)CCC(OCCCCCCCCC)OCCCCCCCCC)CN(CCO)C1)OCCCCCCCCC] |
| CC- 101 | 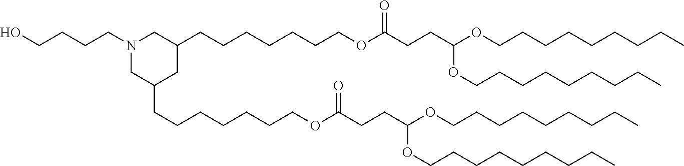 [SMILES: CCCCCCCCCOC(CCC(=O)OCCCCCCCC1CC(CCCCCCCOC(=O)CCC(OCCCCCCCCC)OCCCCCCCCC)CN(CCCCO)C1)OCCCCCCCCC] |
| CC- 102 | 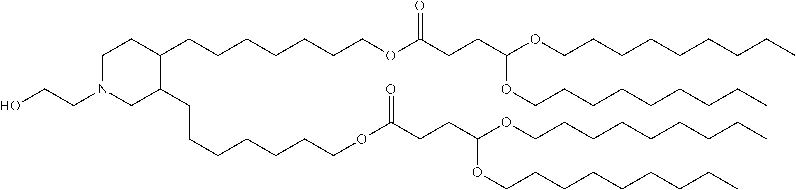 [SMILES: CCCCCCCCCOC(CCC(=O)OCCCCCCCC1CCN(CCO)CC1CCCCCCCOC(=O)CCC(OCCCCCCCCC)OCCCCCCCCC)OCCCCCCCCC] |
| CC- 103 | 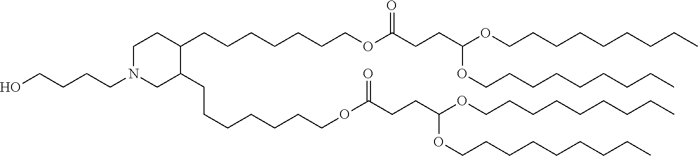 [SMILES: CCCCCCCCCOC(CCC(=O)OCCCCCCCC1CCN(CCCCO)CC1CCCCCCCOC(=O)CCC(OCCCCCCCCC)OCCCCCCCCC)OCCCCCCCCC] |
| CC- 104 | 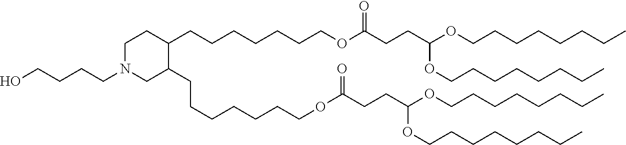 [SMILES: CCCCCCCCOC(CCC(=O)OCCCCCCCC1CCN(CCCCO)CC1CCCCCCCOC(=O)CCC(OCCCCCCCC)OCCCCCCCC)OCCCCCCCC] |
| CC- 105 | 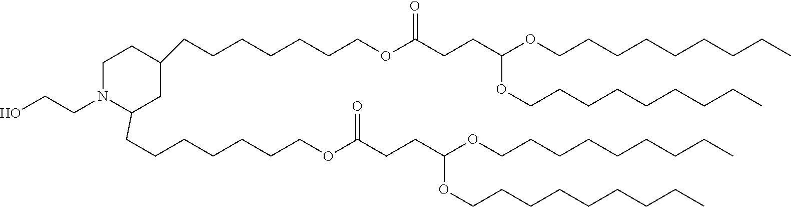 [SMILES: CCCCCCCCCOC(CCC(=O)OCCCCCCCC1CCN(CCO)C(CCCCCCCOC(=O)CCC(OCCCCCCCCC)OCCCCCCCCC)C1)OCCCCCCCCC] |
| CC- 106 | 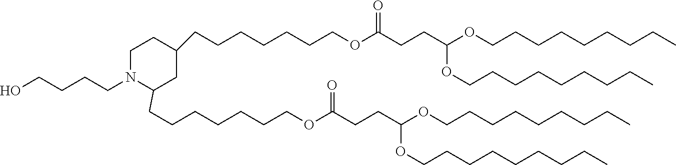 [SMILES: CCCCCCCCCOC(CCC(=O)OCCCCCCCC1CCN(CCCCO)C(CCCCCCCOC(=O)CCC(OCCCCCCCCC)OCCCCCCCCC)C1)OCCCCCCCCC] |
| CC- 107 | 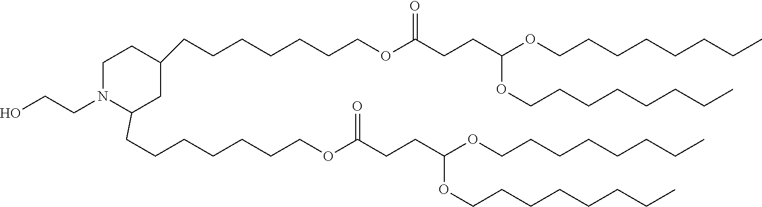 [SMILES: CCCCCCCCOC(CCC(=O)OCCCCCCCC1CCN(CCO)C(CCCCCCCOC(=O)CCC(OCCCCCCCC)OCCCCCCCC)C1)OCCCCCCCC] |

### Series “CT”

Described below are a number of exemplary ionizable lipids of the present disclosure.

In some embodiments, ionizable lipids of the present disclosure have a structure of Formula (CT)

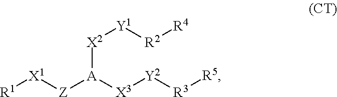

or a pharmaceutically acceptable salt thereof, wherein:
i) A is N; Z is a bond,

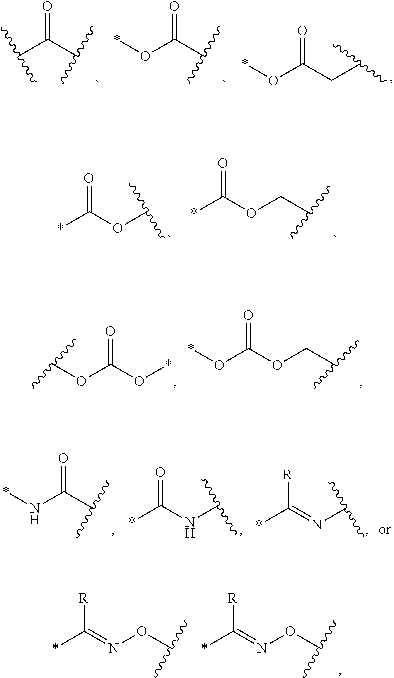

wherein the bond marked with an “*” is attached to X¹; X¹ is optionally substituted C₁-C₆ aliphatic; and R¹ is selected from the group consisting of —OH, —OAc, —NR₂,

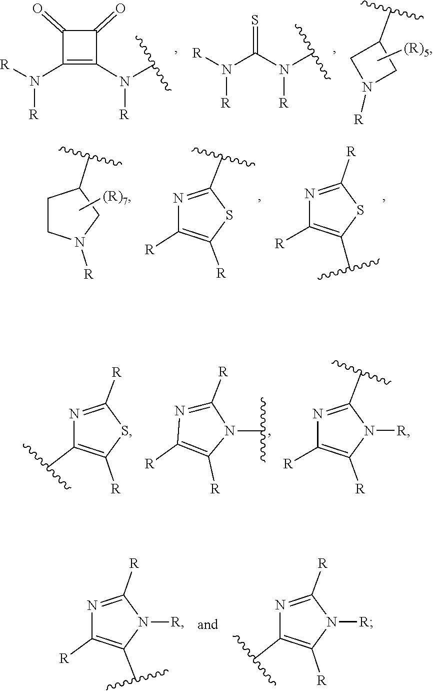

or
ii) A is CH;
Z is

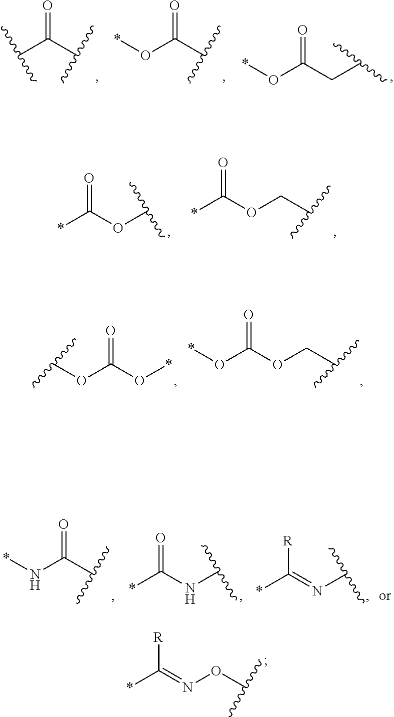

wherein the bond marked with an “*” is attached to X¹;
X¹ is a bond or optionally substituted C₁-C₆ aliphatic;
R¹ is selected from the group consisting of —OH, —OAc, —NR₂,

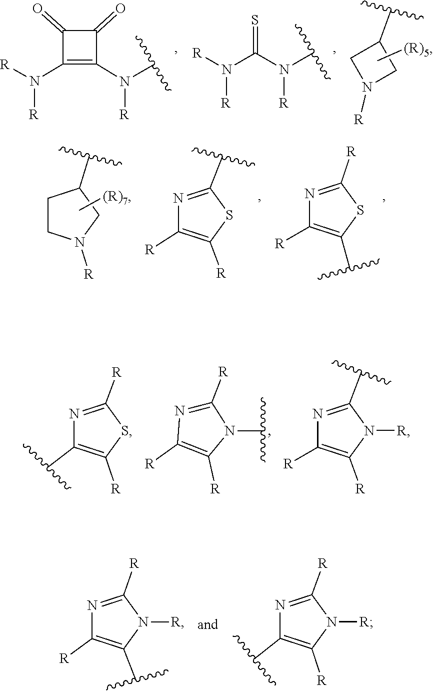

each R is independently —H or C₁-C₆ aliphatic;
X² and X³ are each independently optionally substituted C₁-C₁₂ aliphatic;
Y¹ and Y² are independently selected from the group consisting of

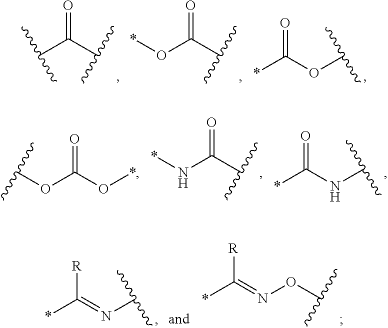

wherein the bond marked with an “*” is attached to X² for Y¹ or X³ for Y²;
R² is a bond or optionally substituted C₁-C₆ aliphatic;
R³ is a bond or optionally substituted C₁-C₆ aliphatic;
R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹), —CH(SR⁸)(SR⁹), —CH(R⁸)(R⁹), —R8, or optionally substituted —C₁-C₆ aliphatic-R⁸;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—;
R⁸ is optionally substituted C₁-C₁₄ aliphatic, wherein at least one methylene linkage is replaced with an optionally substituted divalent radical of a structure selected from

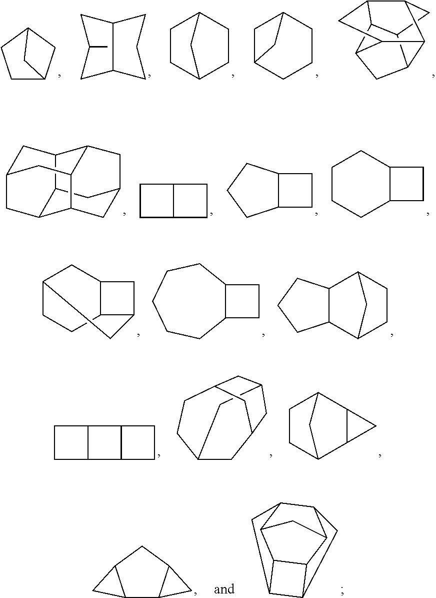

and R⁹ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—.

### Additional Formulae

In certain embodiments, ionizable lipids of the present disclosure have a structure of Formula (CT), wherein the ionizable lipids of the present disclosure have a structure of any one of the following Formulae:

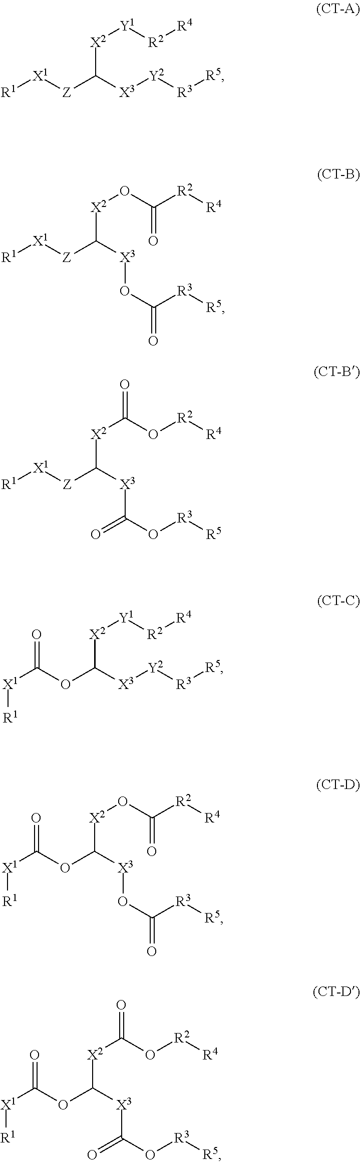

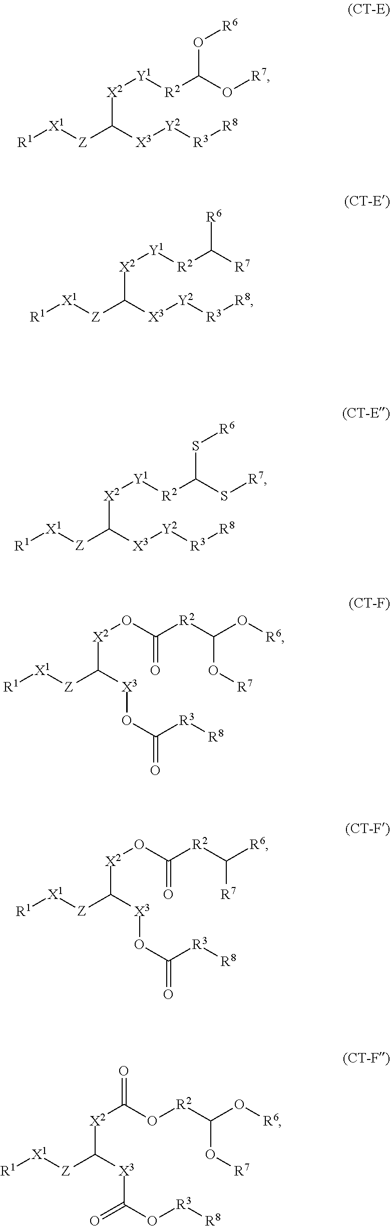

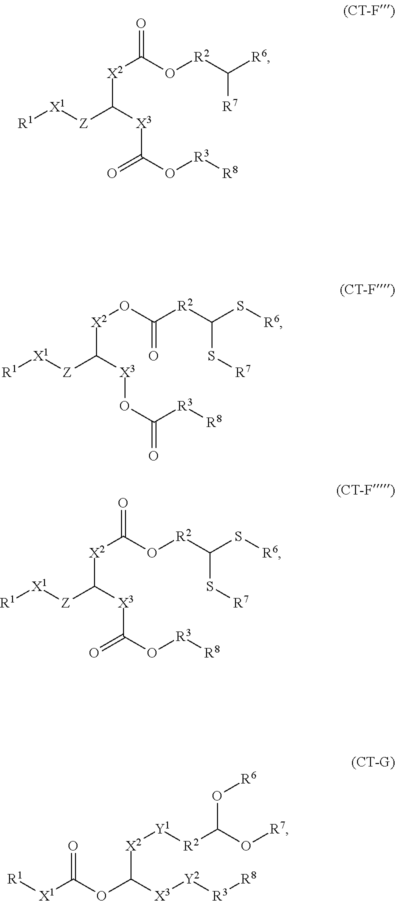

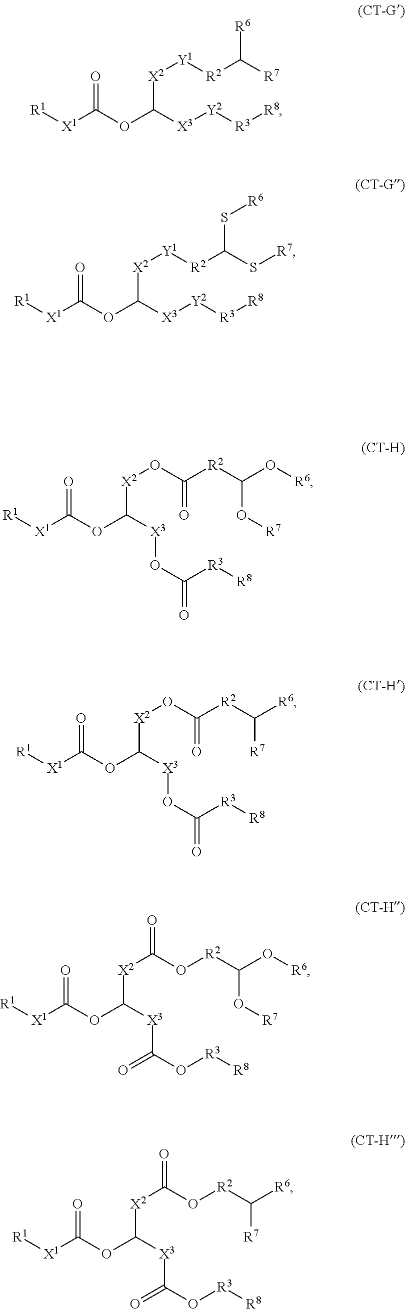

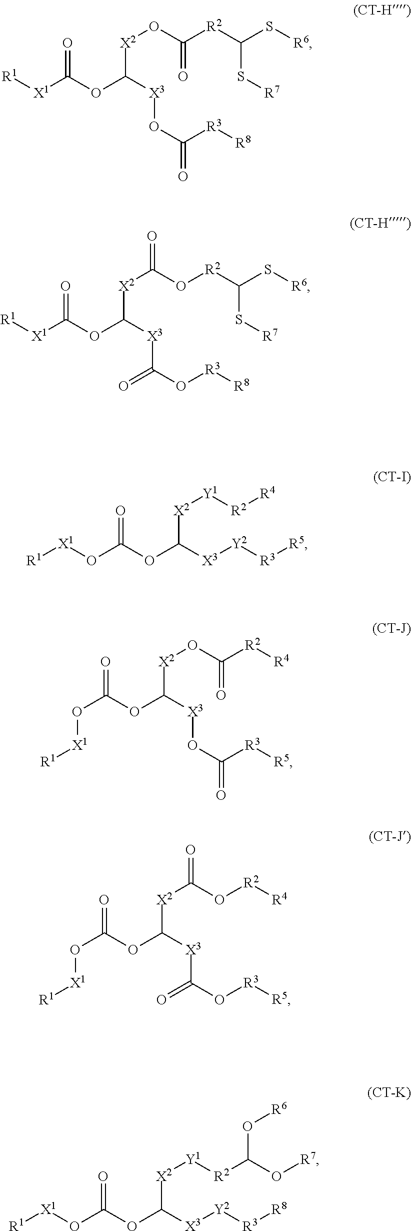

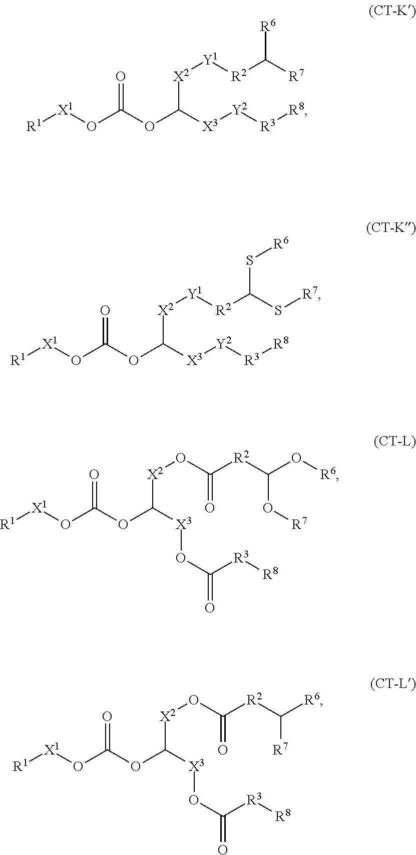

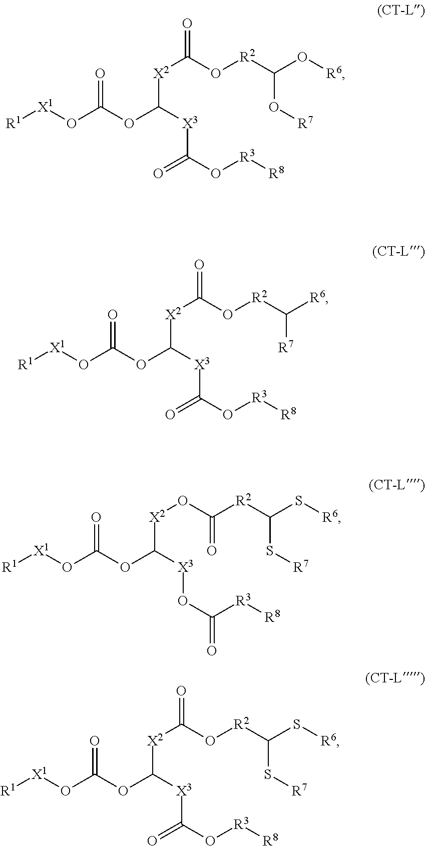

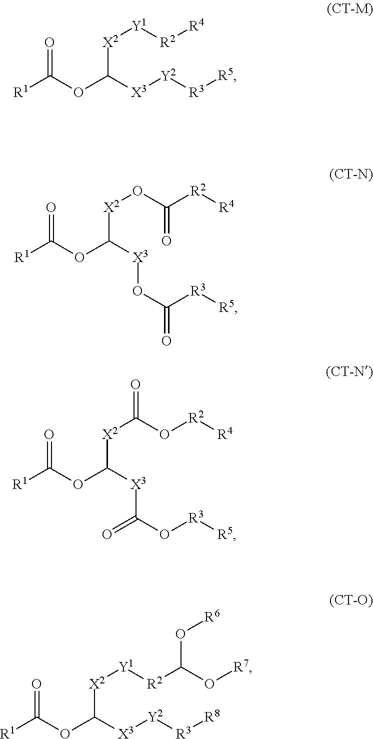

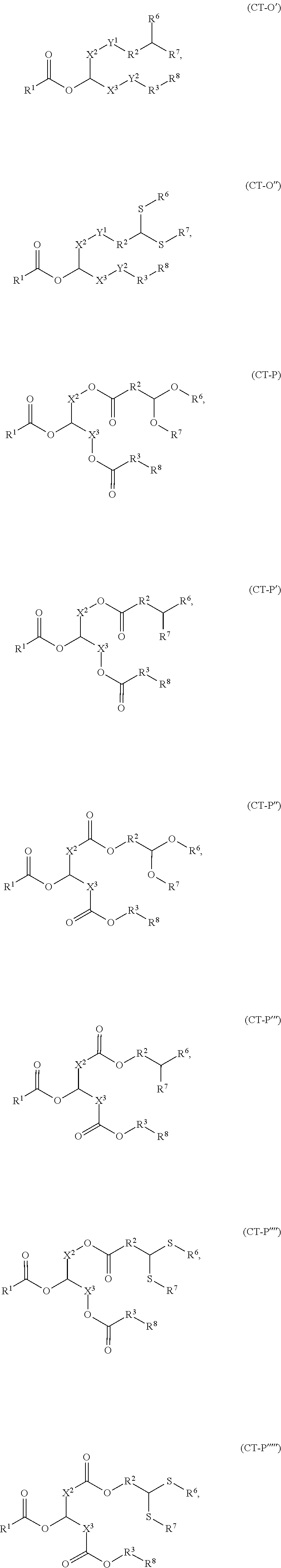

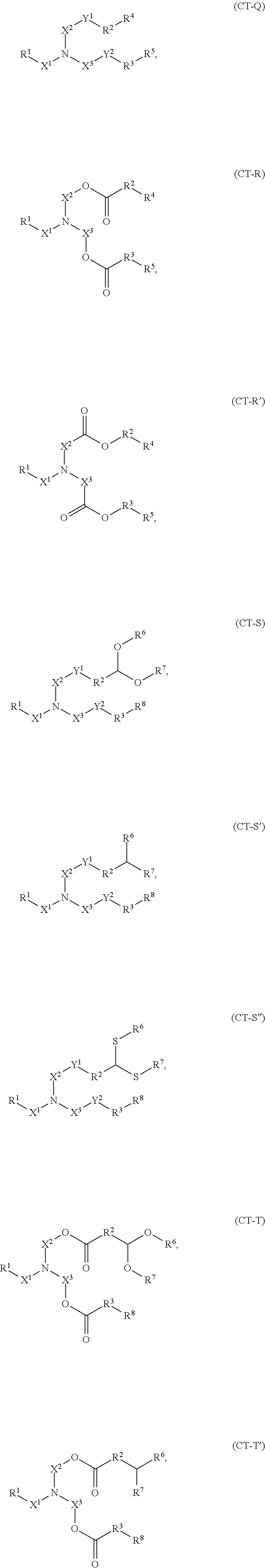

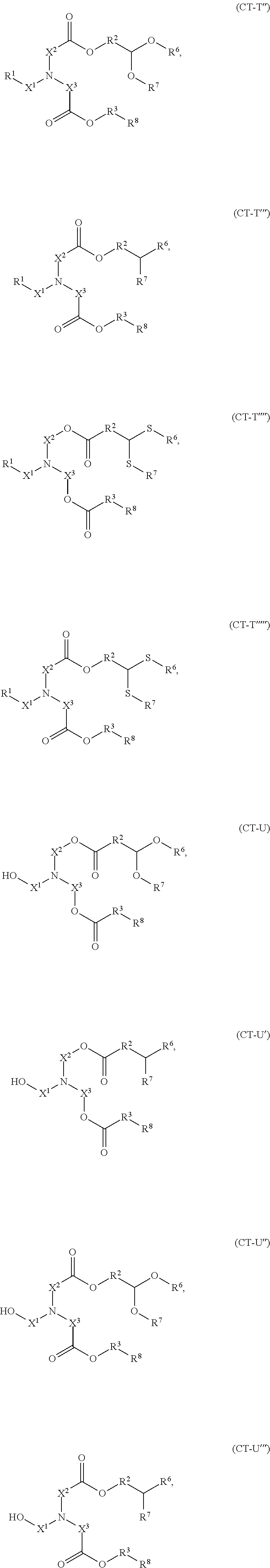

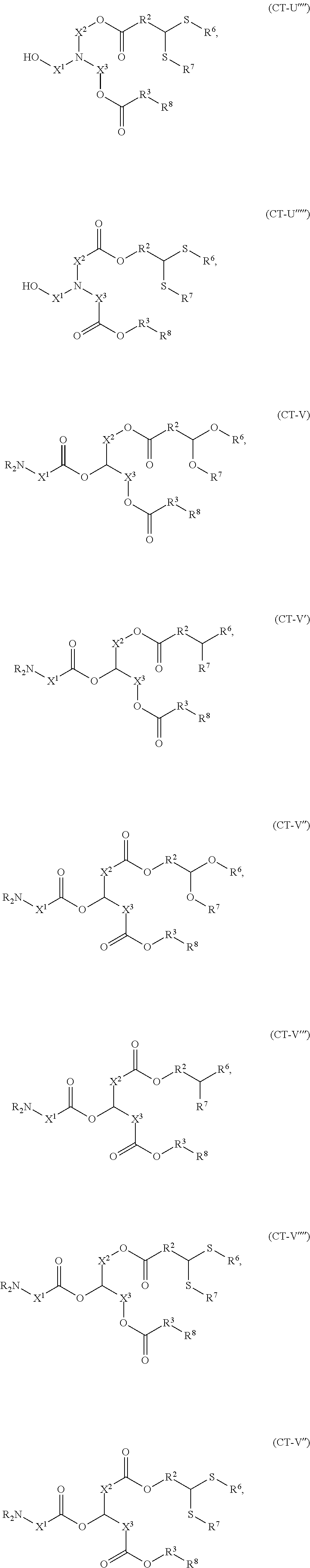

or a pharmaceutically acceptable salt thereof, wherein R¹, R, X¹, Z, X², X³, X⁴, R^{Z}, Y¹. Y², R², R³, R⁴, R⁵, R⁶, R⁷, R⁸, and R⁹ are as described in Formula (CT) or as otherwise described in any embodiments below.

### A

As disclosed in Formula (CT), in certain embodiments, A is CH or N. In certain embodiments, A is CH. In certain embodiments, A is N.

### Z

As disclosed in Formula (CT), in certain embodiments, Z is

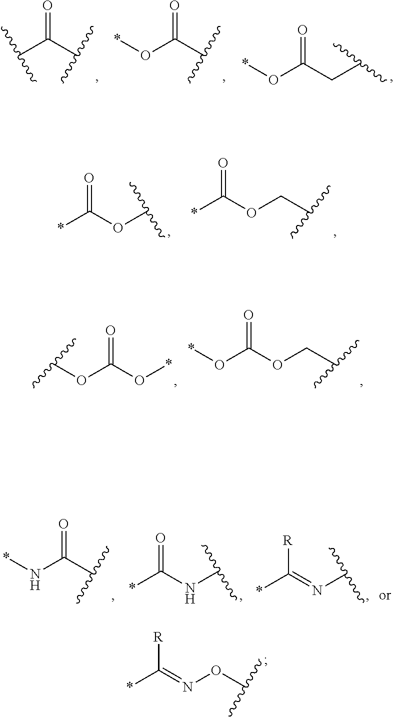

wherein the bond marked with an “*” is attached to X. In certain embodiments, Z is

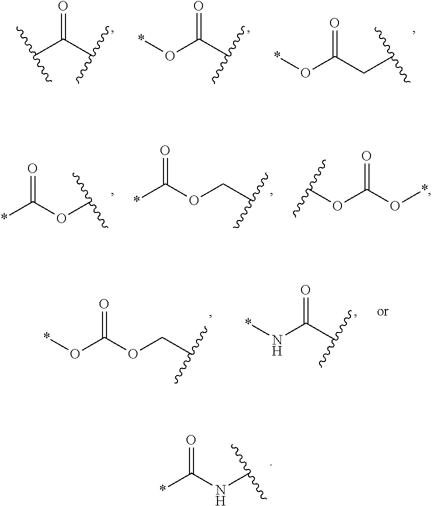

In certain embodiments, Z is

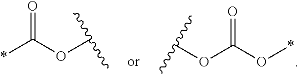 [SMILES: *OC(*)=O ; *OC(=O)O*]

In certain embodiments, Z is

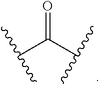 [SMILES: *C(*)=O]

In certain embodiments, Z is

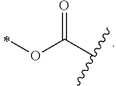

In certain embodiments, Z is

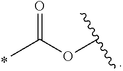 [SMILES: *OC(C)=O]

In certain embodiments, Z is

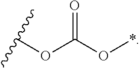

In certain embodiments, Z is

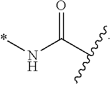 [SMILES: *NC(*)=O ; "."]

In certain embodiment, Z is

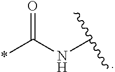 [SMILES: *NC(*)=O]

In certain embodiments, Z is

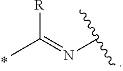

In certain embodiments, Z is

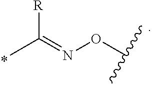

In certain embodiments, Z is

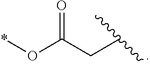 [SMILES: *CC(=O)O*]

In certain embodiments, Z is

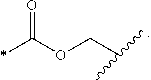 [SMILES: *COC(*)=O]

In certain embodiments, Z is

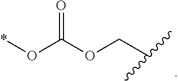 [SMILES: *COC(=O)O*]

As disclosed in Formula (CT), in certain embodiments wherein A is N, Z is a bond.

### X¹

As disclosed in Formula (CT), in certain embodiments wherein A is N, X¹ is optionally substituted C₁-C₆ aliphatic. In certain embodiments wherein A is N, X¹ is unsubstituted C₁-C₆ aliphatic. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

As disclosed in Formula (CT), in certain embodiments wherein A is CH, X¹ is a bond or optionally substituted C₁-C₆ aliphatic. In certain embodiments, X¹ is a bond. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### R¹

As disclosed in Formula (CT), in certain embodiments R¹ is selected from —OH, —OAc, —NR₂,

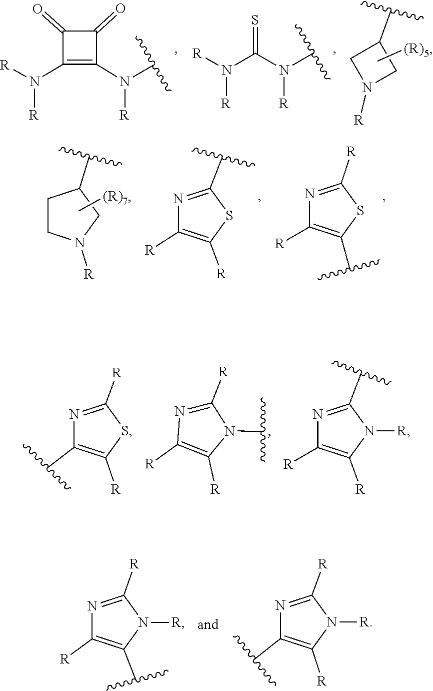

In certain embodiments, R¹ is —OH. In certain embodiments, R¹ is —OAc. In certain embodiments, R¹ is —NR₂. In certain embodiments, R¹ is —NH₂. In certain embodiments, R¹ is —NMe₂. In certain embodiments, R¹ is —NEt₂. In certain embodiments, R¹ is

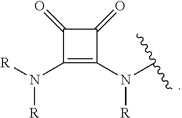

In certain embodiments, R¹ is

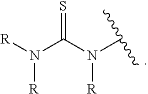

In certain embodiments, R¹ is

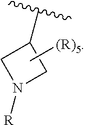

In certain embodiments, R¹ is

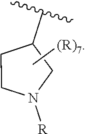

In certain embodiments, R¹ is

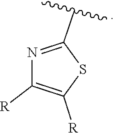

In certain embodiments, R¹ is

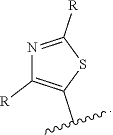

In certain embodiments, R¹ is

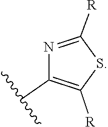

In certain embodiments, R¹ is

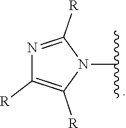

In certain embodiments, R¹ is

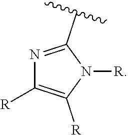

In certain embodiments, R¹ is

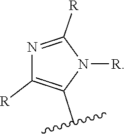

In certain embodiments, R¹ is

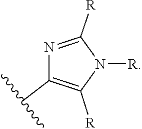

In certain embodiments, R¹ is —(CH₂)₄—, X¹ is —OH, A is N and Z is a bond. In certain embodiments, R¹ is —(CH₂)₄—, X¹ is —OH, A is N and Z is a bond.

X² and X³

As disclosed in Formula (CT), in certain embodiments, X² and X³ are each independently optionally substituted C₁-C₁₂ aliphatic. In certain embodiments, X² and X³ are the same. In certain embodiments, X² and X³ are different.

In certain embodiments, X² is an optionally substituted C₁-C₁₂ alkylene. In certain embodiments, X² is an optionally substituted C₁-C₁₂ alkenylene. In certain embodiments, X² is an optionally substituted C₁-C₁₀ aliphatic. In certain embodiments, X² is an optionally substituted C₁-C₁₀ alkylene. In certain embodiments, X² is an optionally substituted C₁-C₁₀ alkenylene. In certain embodiments, X² is an optionally substituted C₁-C₈ aliphatic. In certain embodiments, X² is an optionally substituted C₁-C₈ alkylene. In certain embodiments, X² is an optionally substituted C₁-C₈ alkenylene. In certain embodiments, X² is an optionally substituted C₁-C₆ aliphatic. In certain embodiments, X² is an optionally substituted C₁-C₆ alkylene. In certain embodiments, X² is an optionally substituted C₁-C₆ alkenylene. In certain embodiments, X² is an optionally substituted C₂-C₁₂ aliphatic. In certain embodiments, X² is an optionally substituted C₂-C₁₂ alkylene. In certain embodiments, X² is an optionally substituted C₂-C₁₂ alkenylene. In certain embodiments, X² is an optionally substituted C₄-C₁₂ aliphatic. In certain embodiments, X² is an optionally substituted C₄-C₁₂ alkylene. In certain embodiments, X² is an optionally substituted C₄-C₁₂ alkenylene. In certain embodiments, X² is an optionally substituted C₄-C₁₀ aliphatic. In certain embodiments, X² is an optionally substituted C₄-C₁₀ alkylene. In certain embodiments, X² is an optionally substituted C₄-C₁₀ alkenylene. In certain embodiments, X² is an optionally substituted C₆-C₈ aliphatic. In certain embodiments, X² is an optionally substituted C₆-C₈ alkylene. In certain embodiments, X² is an optionally substituted C₆-C₈ alkenylene. In certain embodiments, X² is —(CH₂)—. In certain embodiments, X² is —(CH₂)₂—. In certain embodiments, X² is —(CH₂)₃—. In certain embodiments, X² is —(CH₂)₄—. In certain embodiments, X² is —(CH₂)₅—. In certain embodiments, X² is —(CH₂)₆—. In certain embodiments, X² is —(CH₂)₇—. In certain embodiments, X² is —(CH₂)₇—. In certain embodiments, X² is —(CH₂)₉—. In certain embodiments, X² is —(CH₂)₁₀—.

In certain embodiments, X³ is an optionally substituted C₁-C₁₂ alkylene. In certain embodiments, X³ is an optionally substituted C₁-C₁₂ alkenylene. In certain embodiments, X³ is an optionally substituted C₁-C₁₀ aliphatic. In certain embodiments, X³ is an optionally substituted C₁-C₁₀ alkylene. In certain embodiments, X³ is an optionally substituted C₁-C₁₀ alkenylene. In certain embodiments, X³ is an optionally substituted C₁-C₈ aliphatic. In certain embodiments, X³ is an optionally substituted C₁-C₈ alkylene. In certain embodiments, X³ is an optionally substituted C₁-C₈ alkenylene. In certain embodiments, X³ is an optionally substituted C₁-C₆ aliphatic. In certain embodiments, X³ is an optionally substituted C₁-C₆ alkylene. In certain embodiments, X³ is an optionally substituted C₁-C₆ alkenylene. In certain embodiments, X³ is an optionally substituted C₂-C₁₂ aliphatic. In certain embodiments, X³ is an optionally substituted C₂-C₁₂ alkylene. In certain embodiments, X³ is an optionally substituted C₂-C₁₂ alkenylene. In certain embodiments, X³ is an optionally substituted C₄-C₁₂ aliphatic. In certain embodiments, X³ is an optionally substituted C₄-C₁₂ alkylene. In certain embodiments, X³ is an optionally substituted C₄-C₁₂ alkenylene. In certain embodiments, X³ is an optionally substituted C₄-C₁₀ aliphatic. In certain embodiments, X³ is an optionally substituted C₄-C₁₀ alkylene. In certain embodiments, X³ is an optionally substituted C₄-C₁₀ alkenylene. In certain embodiments, X³ is an optionally substituted C₆-C₈ aliphatic. In certain embodiments, X³ is an optionally substituted C₆-C₈ alkylene. In certain embodiments, X³ is an optionally substituted C₆-C₈ alkenylene. In certain embodiments, X³ is —(CH₂)—. In certain embodiments, X³ is —(CH₂)₂—. In certain embodiments, X³ is —(CH₂)₃—. In certain embodiments, X³ is —(CH₂)₄—. In certain embodiments, X³ is —(CH₂)₅—. In certain embodiments, X³ is —(CH₂)₆—. In certain embodiments, X³ is —(CH₂)₇—. In certain embodiments, X³ is —(CH₂)₈—. In certain embodiments, X³ is —(CH₂)₉—. In certain embodiments, X³ is —(CH₂)₁₀—.

In certain embodiments, X² and X³ are both optionally substituted C₆-C₈ alkylene. In certain embodiments, X² and X³ are both —(CH₂)₈—. In certain embodiments, X² and X³ are both —(CH₂)₇—. In certain embodiments, X² and X³ are both —(CH₂)₆—.

Y¹ and Y²

As disclosed in Formula (CT), in certain embodiments, Y¹ and Y² are each independently

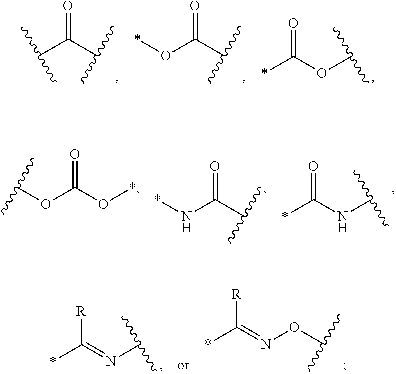

wherein the bond marked with an “*” is attached to X² for Y¹ or X³ for Y². In certain embodiments, Y¹ and Y² are the same. In certain embodiments, Y¹ and Y² are different.

In certain embodiments, Y¹ and Y² are each independently

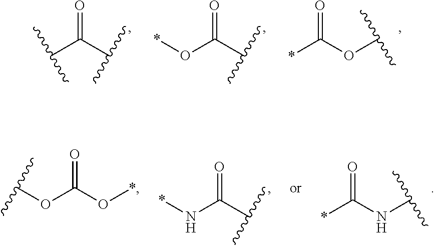

In certain embodiments, Y¹ and Y² are each independently or

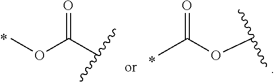

In certain embodiments, Y¹ is

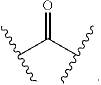 [SMILES: *C(*)=O]

In certain embodiments, Y¹ is

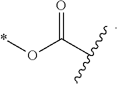 [SMILES: *OC(*)=O]

In certain embodiments, Y¹ is

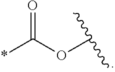 [SMILES: *OC(*)=O]

In certain embodiments, Y is

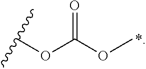

In certain embodiments, Y¹ is

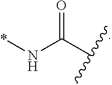 [SMILES: *NC(*)=O ; "."]

In certain embodiments, Y¹ is

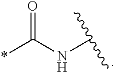 [SMILES: *NC(*)=O]

In certain embodiments, Y¹ is

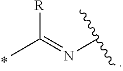

In certain embodiments, Y¹ is

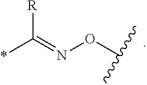

In certain embodiments, Y² is

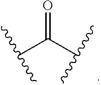 [SMILES: *C(*)=O]

In certain embodiments, Y² is

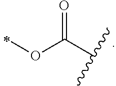

In certain embodiments, Y² is

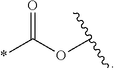 [SMILES: *OC(*)=O]

In certain embodiments, Y² is

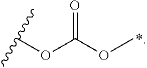

In certain embodiments, Y² is

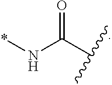 [SMILES: *NC(*)=O ; "."]

In certain embodiments, Y² is

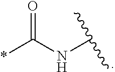 [SMILES: *NC(*)=O]

In certain embodiments, Y² is

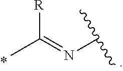

In certain embodiments, Y² is

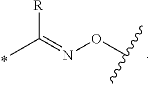

In certain embodiments, Y¹ and Y² are both

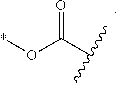 [SMILES: *OC(*)=O]

In certain embodiments, Y¹ and Y² are both

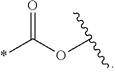 [SMILES: *OC(*)=O]

### R²

As disclosed in Formula (CT), in certain embodiments, R² is a bond or optionally substituted C₁-C₆ aliphatic. In certain embodiments, R² is a bond. In certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (CT), in certain embodiments, R³ is a bond or optionally substituted C₁-C₆ aliphatic. In certain embodiments, R² is a bond. In certain embodiments, R² is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different.

### R⁴

As disclosed in Formula (CT), in certain embodiments, R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—. R⁴ is optionally substituted C₁-C₁₄ aliphatic. In certain embodiments, R⁴ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷).

In certain embodiments, one of the methylene linkages of R⁴ is replaced with an optionally substituted C₃-C₈ cycloalkylenyl, or an optionally substituted bridged bicyclic or multicyclic C₅-C₁₄ cycloalkylenyl. In certain embodiments, one of the methylene linkages of R⁴ is replaced with an optionally substituted C₃-C₈ cycloalkylenyl. In certain embodiments, one of the methylene linkages of R⁴ is replaced with an optionally substituted bridged bicyclic or multicyclic C₅-C₁₄ cycloalkylenyl. In certain embodiments, one of the methylene linkages of R⁴ is replaced with an optionally substituted bridged bicyclic or multicyclic C₅-C₁₂ cycloalkylenyl. In certain embodiments, the optionally substituted bridged bicyclic or multicyclic C₅-C₁₄ cycloalkylenyl is selected from:

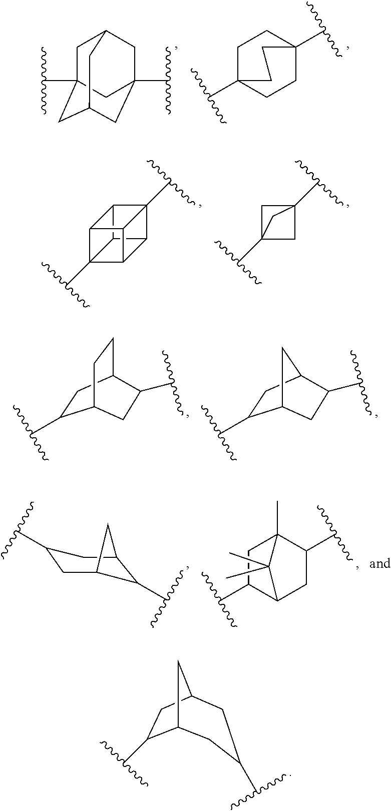

In certain embodiments, the optionally substituted bridged bicyclic or multicyclic C₅-C₁₄ cycloalkylenyl is a divalent radical of a structure selected from:

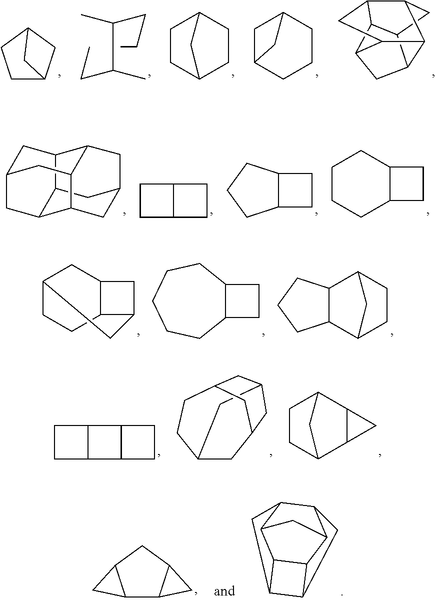

In certain embodiments, R⁴ is selected from

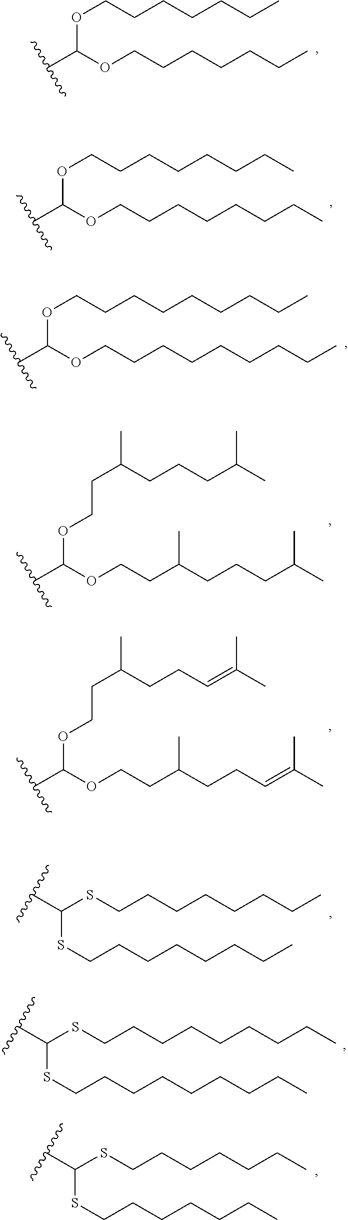

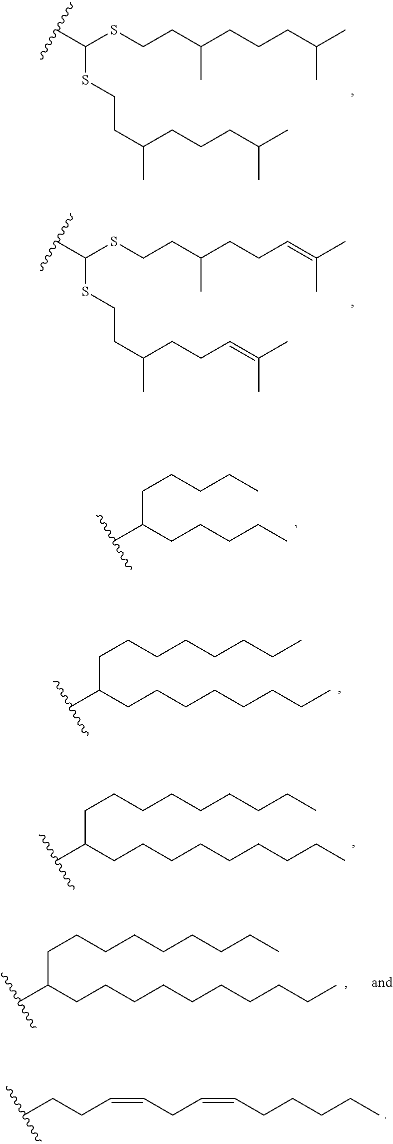

In certain embodiments, R⁴ is selected from

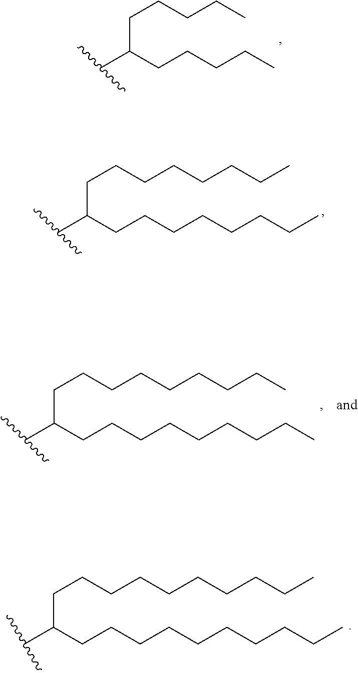 [SMILES: *C(CCCCC)CCCCC ; *C(CCCCCCCC)CCCCCCCC ; *C(CCCCCCCCC)CCCCCCCCC ; *C(CCCCCCCCCC)CCCCCCCCCC]

In certain embodiments, R is selected from

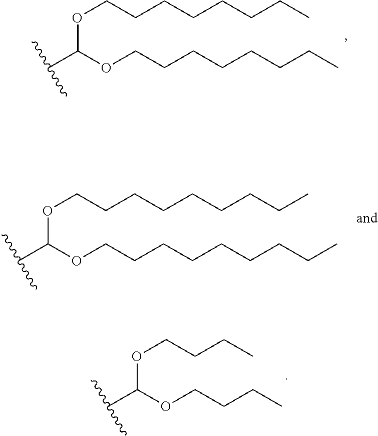 [SMILES: *C(OCCCCCCCC)OCCCCCCCC ; *C(OCCCCCCCCC)OCCCCCCCCC ; *C(OCCCC)OCCCC]

In certain embodiments, R⁴ is

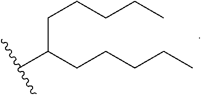 [SMILES: *C(CCCCC)CCCCC ; "."]

### R⁵

As disclosed in Formula (CT), in certain embodiments, R⁵ is —CH(OR)(OR⁹), —CH(SR⁸)(SR⁹), —CH(R⁸)(R⁹), —R⁸, or optionally substituted —C₁-C₆ aliphatic-R⁸. In certain embodiments, R⁵ is —R¹ or optionally substituted —C₁-C₆ aliphatic-R⁸. In certain embodiments, R⁵ is —R⁸. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SRs)(SR⁹).

In certain embodiments, R⁴ and R⁵ are the same. In certain embodiments, R⁴ and R⁵ are different.

R⁶ and R⁷

As disclosed in Formula (CT), in certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkylene. In certain embodiments, R⁶ is optionally substituted C₁-C₁₄ branched alkylene. In certain embodiments, R⁶ is optionally substituted C₁-C₁₄ straight chain alkylene. In certain embodiments, R⁶ is optionally substituted C₁-C₁₄ alkenylene. In certain embodiments, R⁶ is optionally substituted C₁-C₁₄ branched alkenylene. In certain embodiments, R⁶ is optionally substituted C₁-C₁₄ straight chain alkenylene. In certain embodiments, R⁶ is optionally substituted C₆-C₁₀ alkylene. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, or an optionally substituted bridged bicyclic or multicyclic C₅-C₁₄ cycloalkylenyl. In certain embodiments, one of the methylene linkages of R⁶ is replaced with an optionally substituted C₃-C₈ cycloalkylenyl. In certain embodiments, one of the methylene linkages of R⁶ is replaced with an optionally substituted bridged bicyclic or multicyclic C₅-C₁₄ cycloalkylenyl. In certain embodiments, one of the methylene linkages of R⁶ is replaced with an optionally substituted bridged bicyclic or multicyclic C₅-C₁₂ cycloalkylenyl. In certain embodiments, the optionally substituted bridged bicyclic or multicyclic C₅-C₁₄ cycloalkylenyl is selected from:

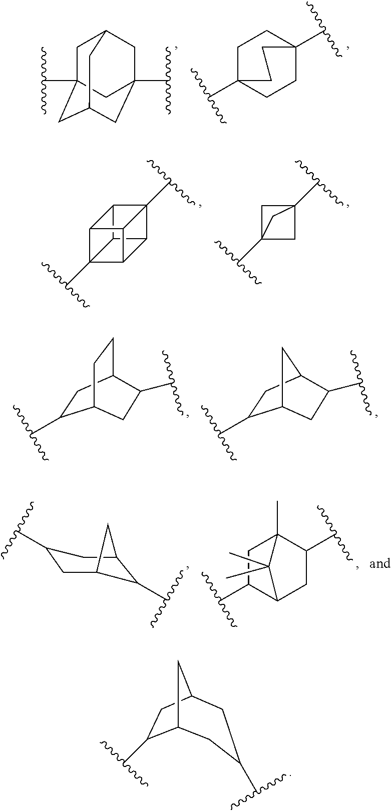

In certain embodiments, the optionally substituted bridged bicyclic or multicyclic C₅-C₁₄ cycloalkylenyl is a divalent radical of a structure selected from:

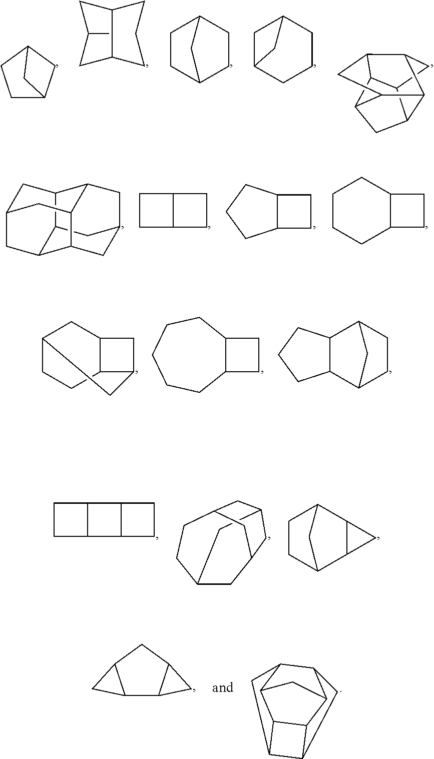

In certain embodiments, R⁷ is optionally substituted C₁-C₁₄ aliphatic. In certain embodiments, R⁷ is optionally substituted C₁-C₁₄ alkylene. In certain embodiments, R⁷ is optionally substituted C₁-C₁₄ branched alkylene. In certain embodiments, R⁷ is optionally substituted C₁-C₁₄ straight chain alkylene. In certain embodiments, R⁷ is optionally substituted C₁-C₁₄ alkenylene. In certain embodiments, R⁷ is optionally substituted C₁-C₁₄ branched alkenylene. In certain embodiments, R⁷ is optionally substituted C₁-C₁₄ straight chain alkenylene. In certain embodiments, R⁷ is optionally substituted C₆-C₁₀ alkylene. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁷ is optionally substituted —(CH₂)₉CH₃.

In certain embodiments, one of the methylene linkages of R⁷ is replaced with an optionally substituted C₃-C₈ cycloalkylenyl, or an optionally substituted bridged bicyclic or multicyclic C₅-C₁₄ cycloalkylenyl. In certain embodiments, one of the methylene linkages of R⁷ is replaced with an optionally substituted C₃-C₈ cycloalkylenyl. In certain embodiments, one of the methylene linkages of R⁷ is replaced with an optionally substituted bridged bicyclic or multicyclic C₅-C₁₄ cycloalkylenyl. In certain embodiments, one of the methylene linkages of R⁷ is replaced with an optionally substituted bridged bicyclic or multicyclic C₅-C₁₂ cycloalkylenyl. In certain embodiments, the optionally substituted bridged bicyclic or multicyclic C₅-C₁₄ cycloalkylenyl is selected from:

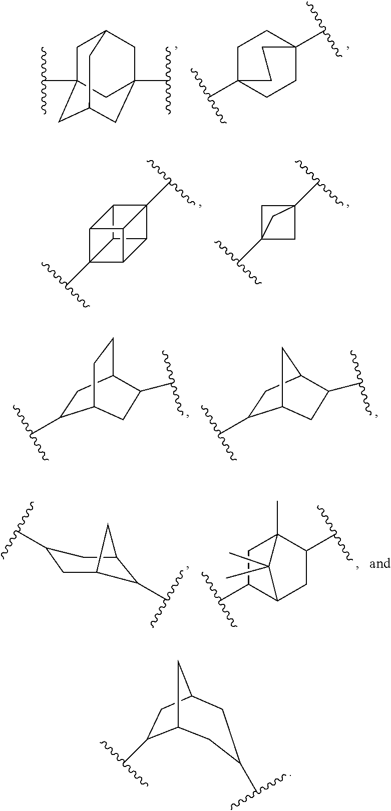

In certain embodiments, the optionally substituted bridged bicyclic or multicyclic C₅-C₁₄ cycloalkylenyl is a divalent radical of a structure selected from:

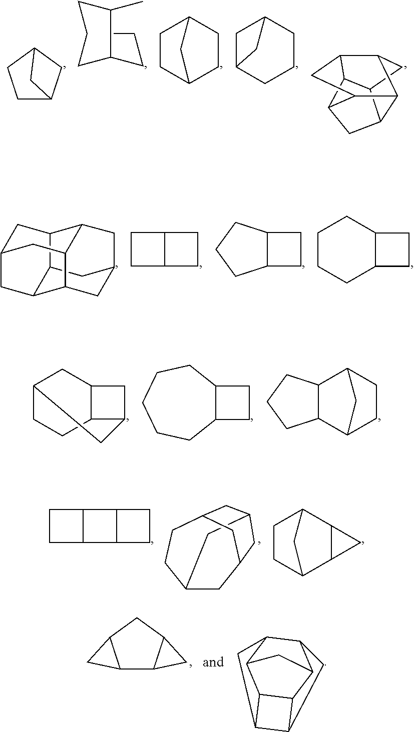

In certain embodiments, each R⁶ and R⁷ are selected from

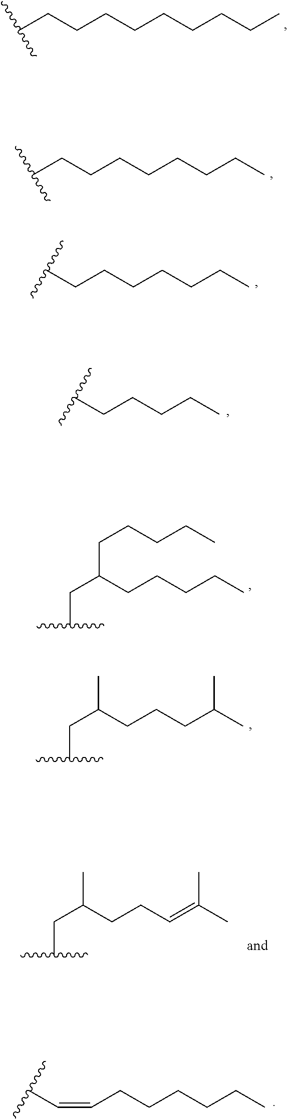

### R⁸

As disclosed in Formula (CT), in certain embodiments, R¹ is optionally substituted C₁-C₁₄ aliphatic, wherein at least one methylene linkage is replaced with an optionally substituted divalent radical of a structure selected from

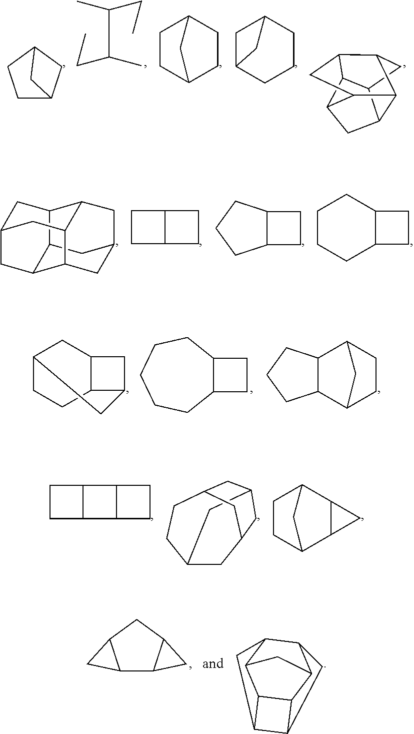

In some embodiments, R⁸ is a structure selected from,

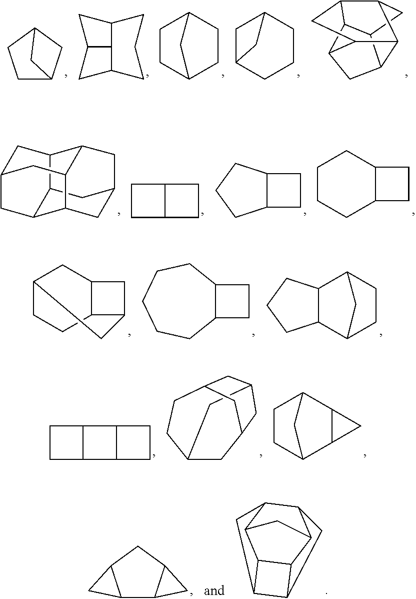

In some embodiments, R⁸ is optionally substituted C₁-C₁₃ alkylene terminated with a monovalent radical of a structure selected from

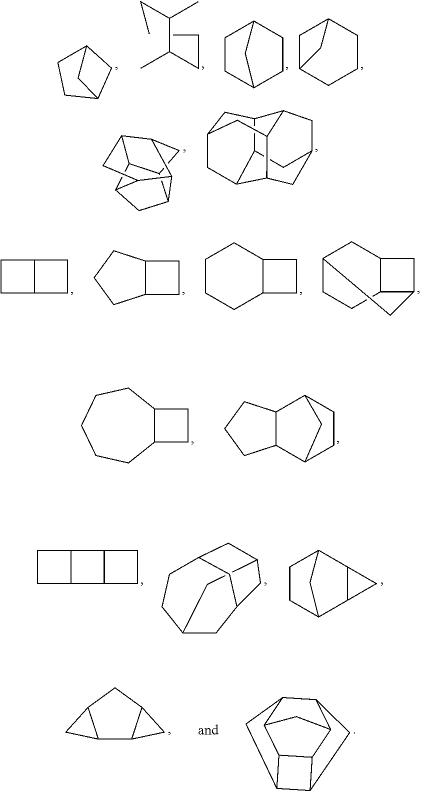

In certain embodiments, R⁸ is a structure selected from

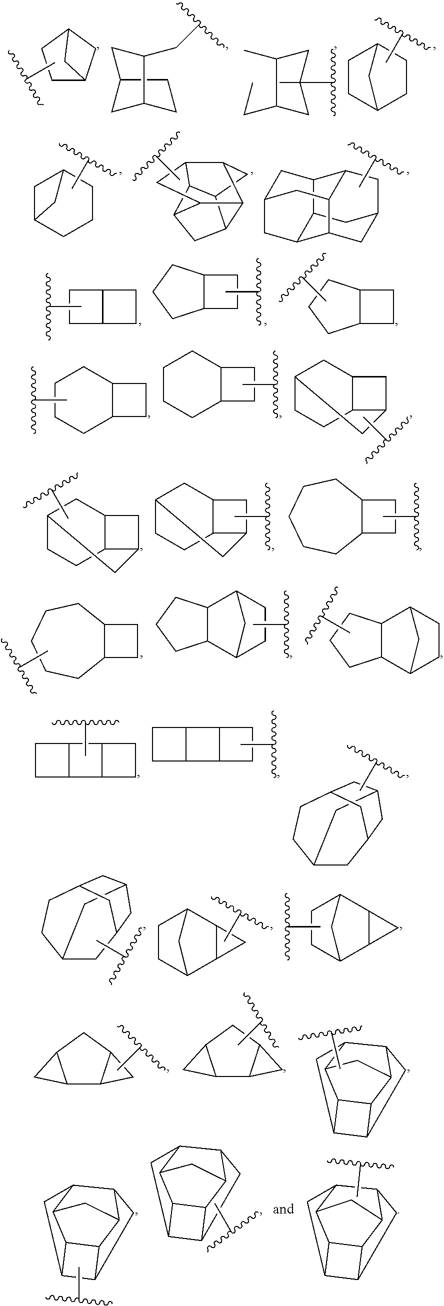

In certain embodiments, R⁸ is optionally substituted C₁-C₁₃ alkylene terminated with a structure selected from

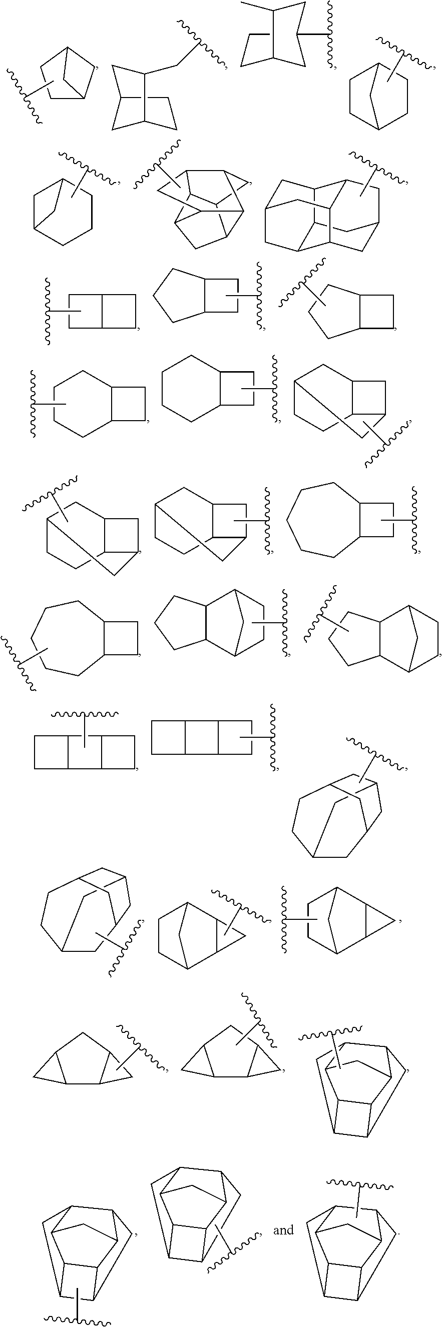

In certain embodiments, R⁸ is a structure selected from

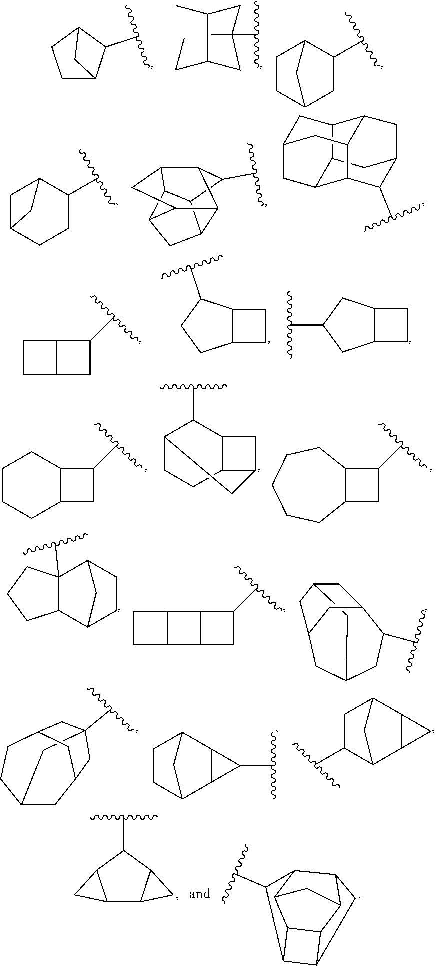

In certain embodiments, R⁸ is optionally substituted C₁-C₁₃ alkylene terminated with a structure selected from

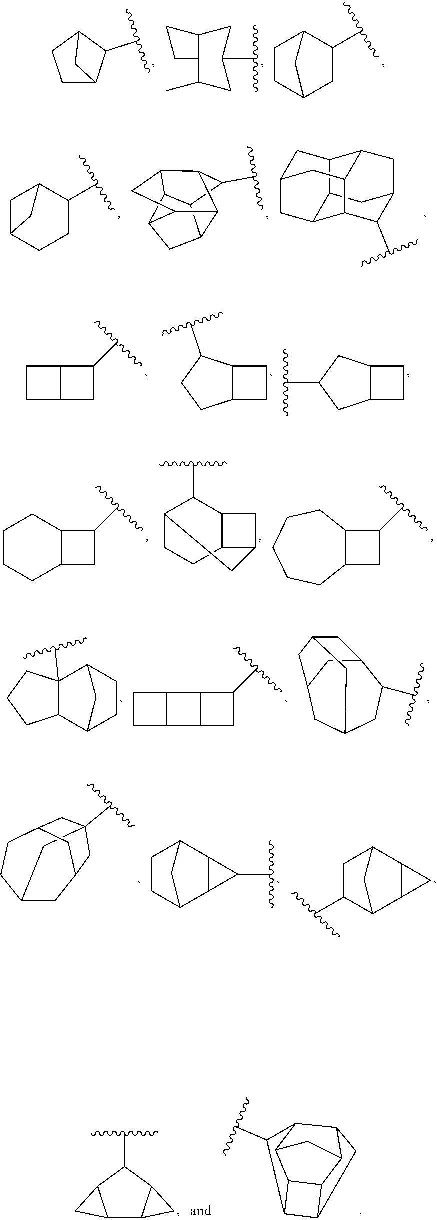

In certain embodiments, R⁸ is an optionally substituted —CH₂—, —(CH₂)₂—, —(CH₂)₃—, —(CH₂)₄—, —(CH₂)₅—, or —(CH₂)₆—, terminated with a structure selected from

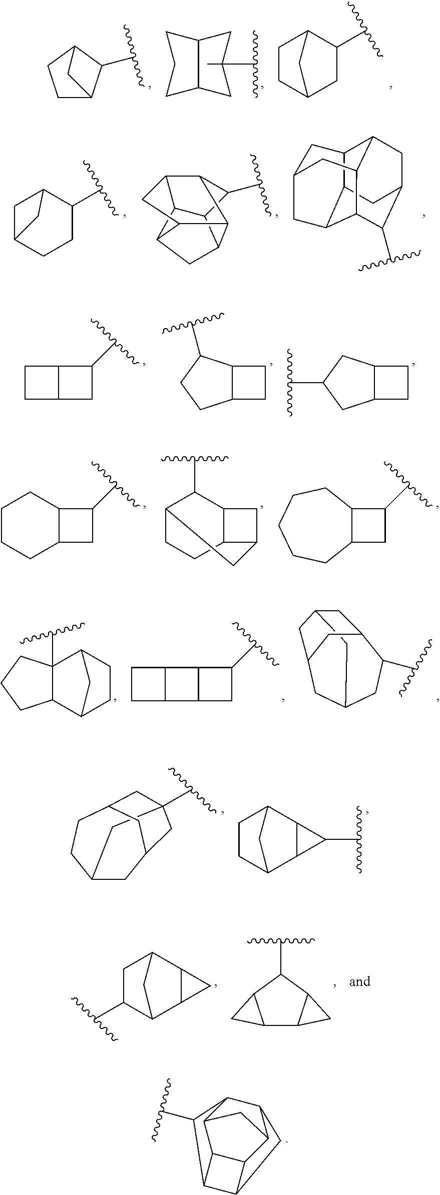

In certain embodiments, R⁸ is a structure selected from the group consisting of

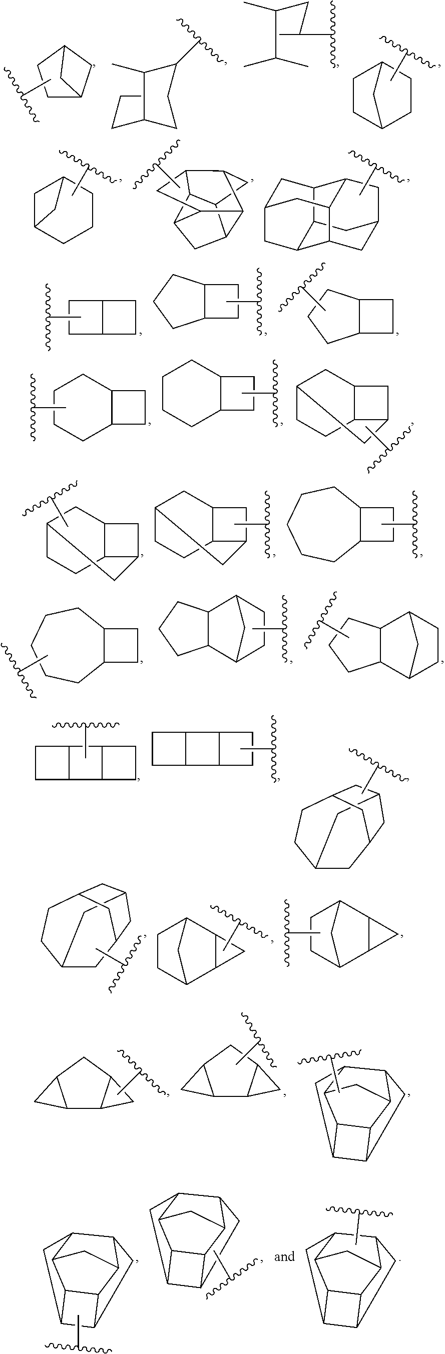

In certain embodiments R⁸ is a structure selected from

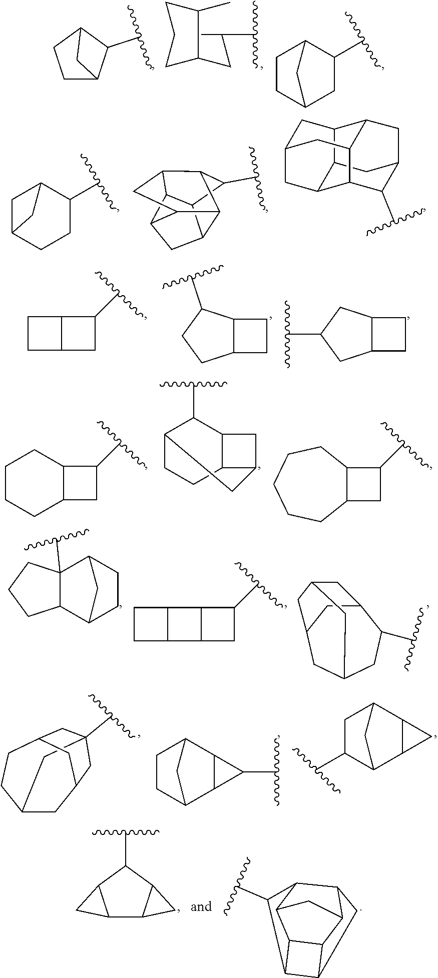

### R⁹

As disclosed in Formula (CT), in certain embodiments, R⁹ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₄ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁹ is optionally substituted C₁-C₁₄ aliphatic. In certain embodiments, R⁹ is optionally substituted C₁-C₁₄ alkylene. In certain embodiments, R⁹ is optionally substituted C₁-C₁₄ branched alkylene. In certain embodiments, R⁹ is optionally substituted C₁-C₁₄ straight chain alkylene. In certain embodiments, R⁶ is optionally substituted C₁-C₁₄ alkenylene. In certain embodiments, R⁹ is optionally substituted C₁-C₁₄ branched alkenylene. In certain embodiments, R⁹ is optionally substituted C₁-C₁₄ straight chain alkenylene. In certain embodiments, R⁹ is optionally substituted C₆-C₁₀ alkylene. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, or an optionally substituted bridged bicyclic or multicyclic C₅-C₁₄ cycloalkylenyl. In certain embodiments, one of the methylene linkages of R⁹ is replaced with an optionally substituted C₃-C₈ cycloalkylenyl. In certain embodiments, one of the methylene linkages of R⁹ is replaced with an optionally substituted bridged bicyclic or multicyclic C₅-C₁₄ cycloalkylenyl. In certain embodiments, one of the methylene linkages of R⁹ is replaced with an optionally substituted bridged bicyclic or multicyclic C₅-C₁₂ cycloalkylenyl. In certain embodiments, the optionally substituted bridged bicyclic or multicyclic C₅-C₁₄ cycloalkylenyl is selected from:

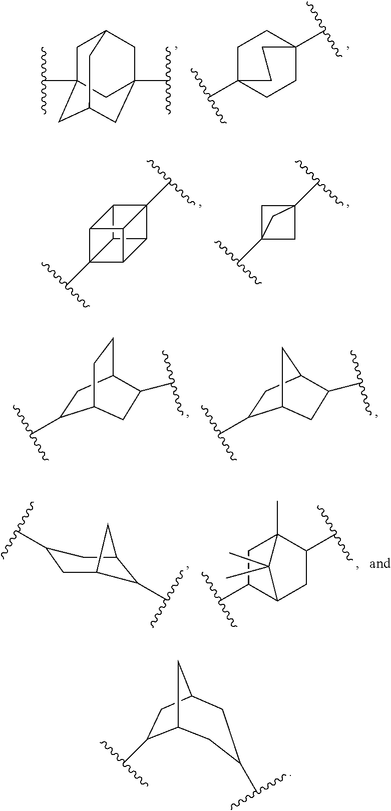

In certain embodiments, the optionally substituted bridged bicyclic or multicyclic C₅-C₁₄ cycloalkylenyl is a divalent radical of a structure selected from:

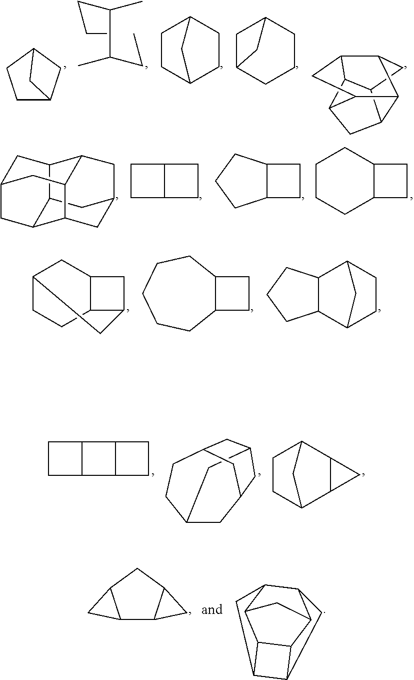

In certain embodiments, R⁹ is selected from

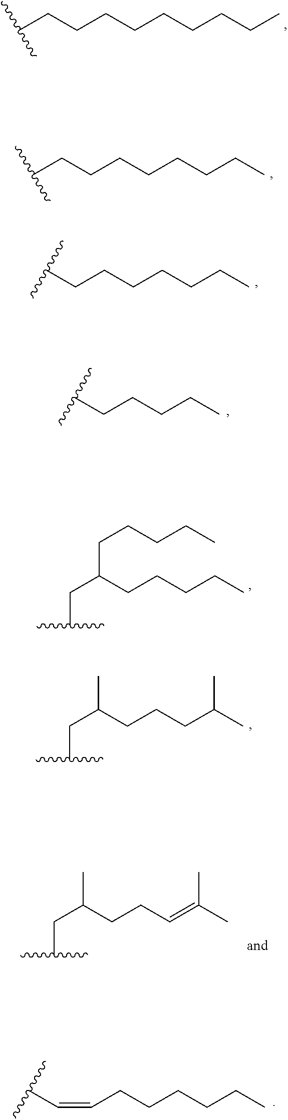

In some embodiments, R⁵ is optionally substituted —C₁-C₆ aliphatic-R⁸ or —R⁸ and R⁹ is absent.

In some embodiments, Lipids of the Present Disclosure are selected from any lipid in Table (I-K) below or a pharmaceutically acceptable salt thereof:

TABLE (I-K)
Non-Limiting Examples of Ionizable Lipids of the Present Disclosure

| Comp No. | Structure |
|---|---|
| CT-1 | 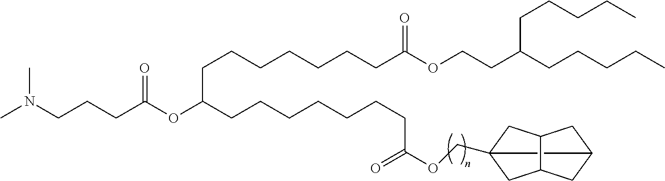 |
| CT-2 | 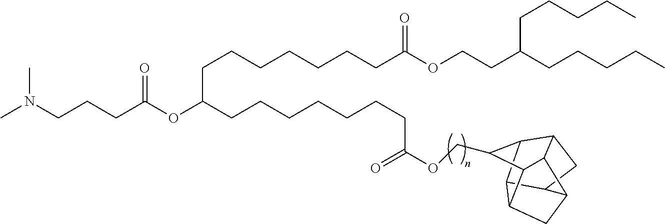 |
| CT-3 | 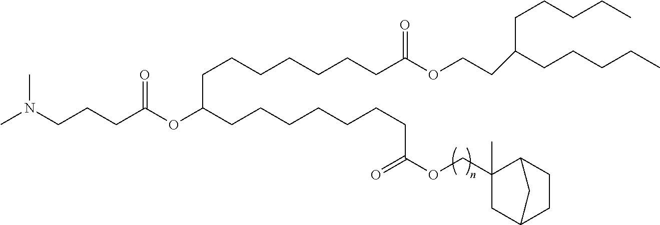 |
| CT-4 | 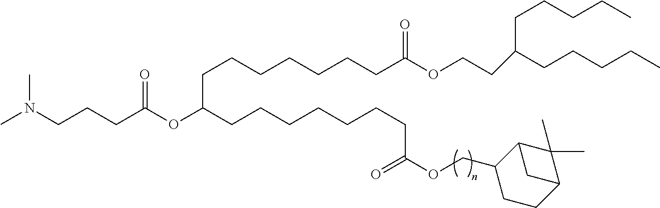 |
| CT-5 | 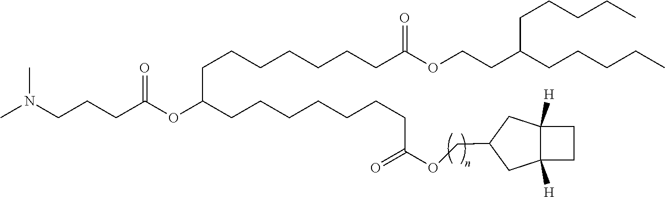 |
| CT-6 | 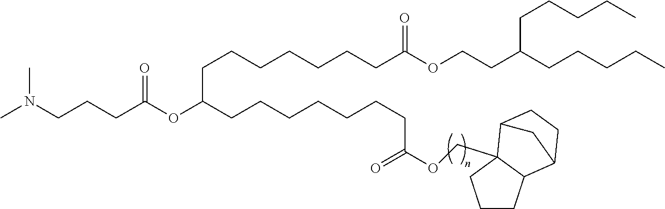 |
| CT-7 | 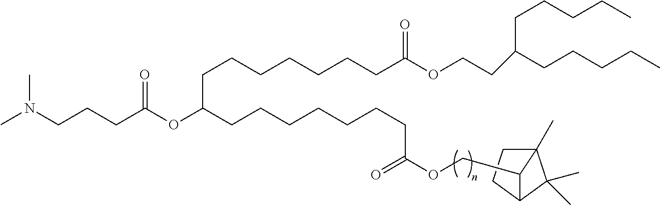 |
| CT-8 | 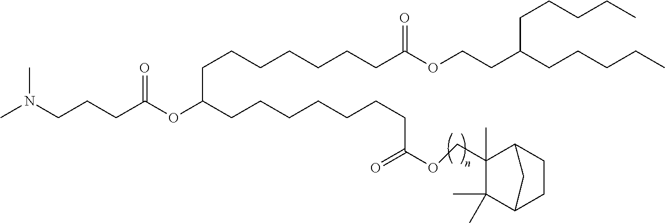 |
| CT-9 | 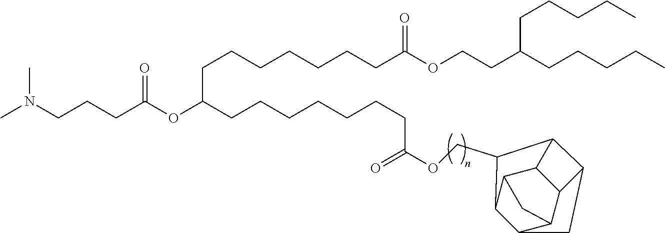 |
| CT-10 | 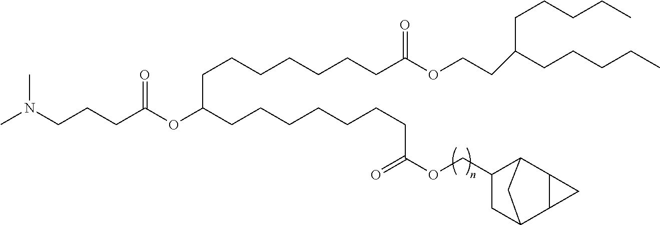 |
| CT-11 | 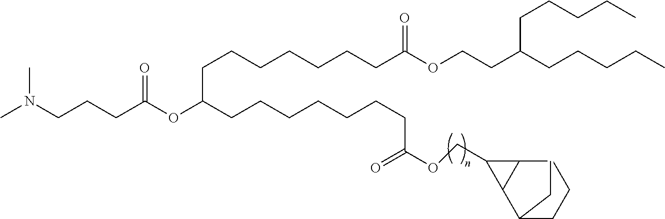 |
| CT-12 | 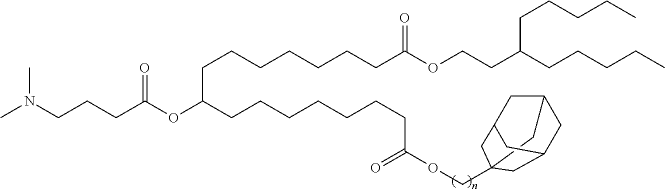 |
| CT-13 | 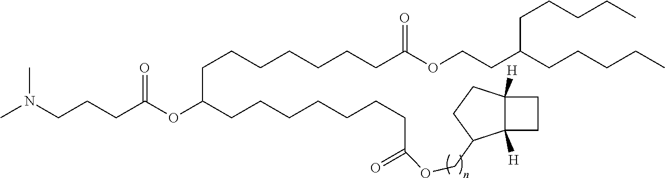 |
| CT-14 | 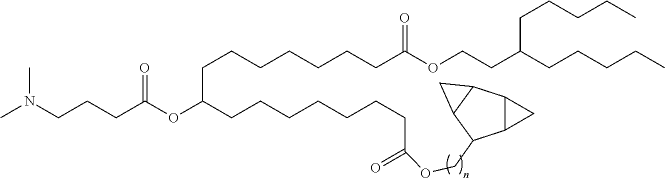 |
| CT-15 | 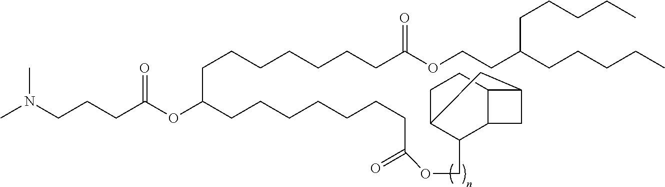 |
| CT-16 | 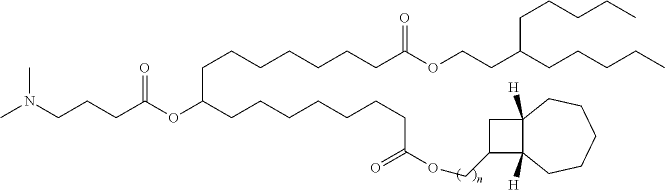 |
| CT-17 | 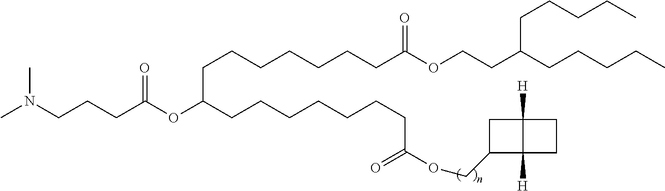 |
| CT-18 | 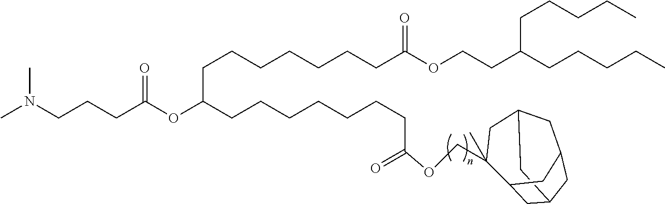 |
| CT-19 | 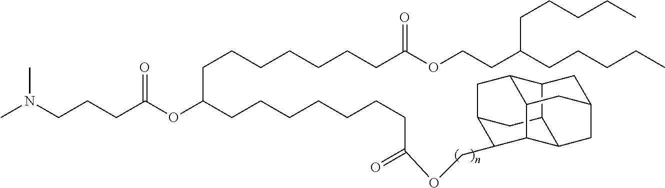 |
| CT-20 | 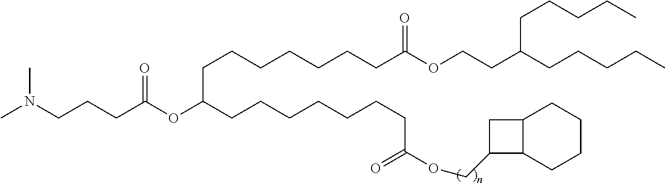 |
| CT-21 | 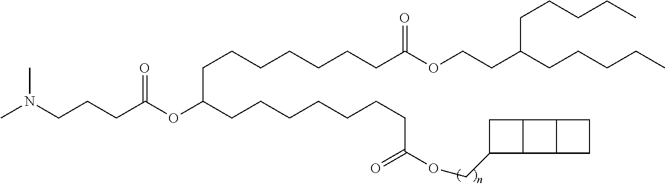 |
| CT-22 | 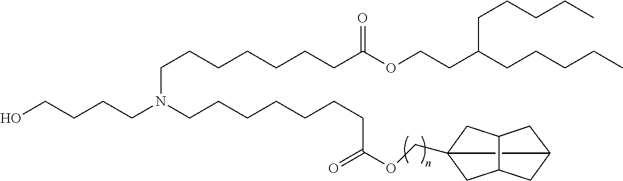 |
| CT-23 | 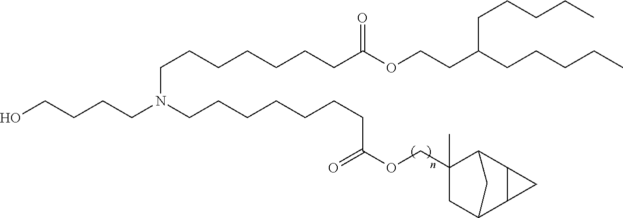 |
| CT-24 | 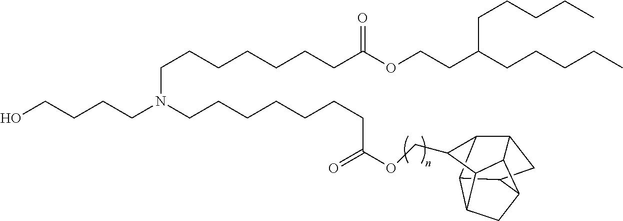 |
| CT-25 | 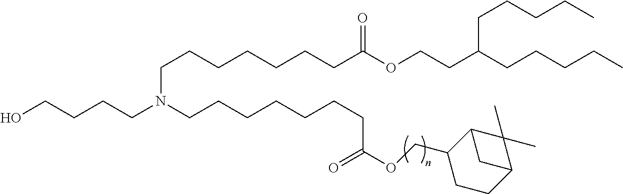 |
| CT-26 | 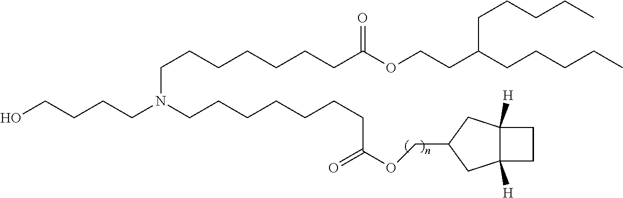 |
| CT-27 | 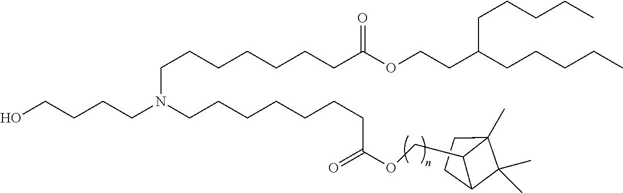 |
| CT-28 | 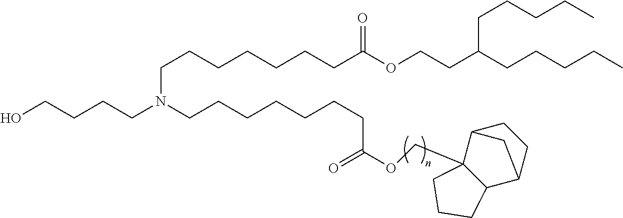 |
| CT-29 | 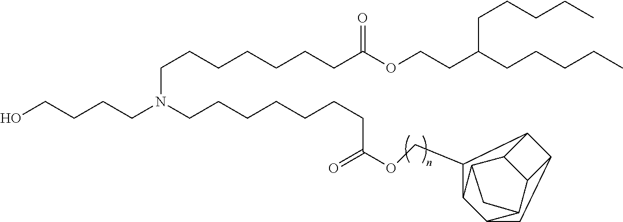 |
| CT-30 | 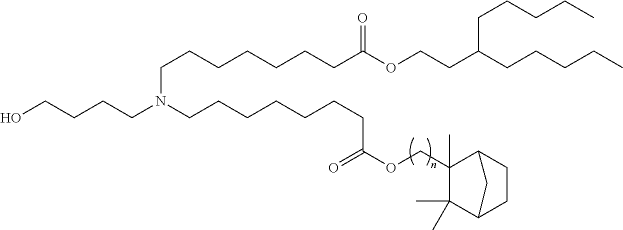 |
| CT-31 | 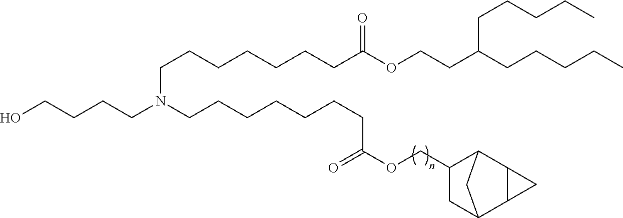 |
| CT-32 | 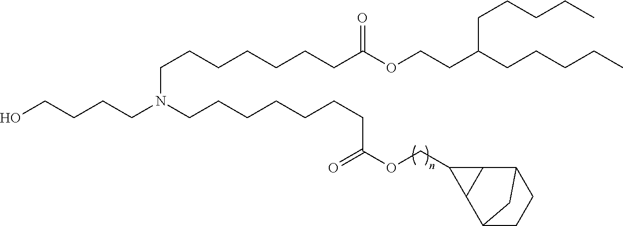 |
| CT-33 | 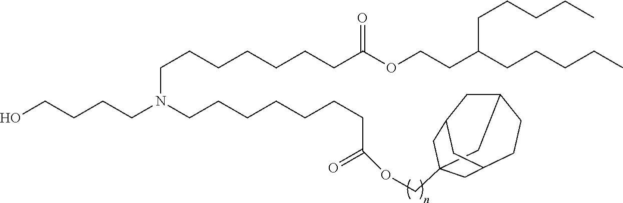 |
| CT-34 | 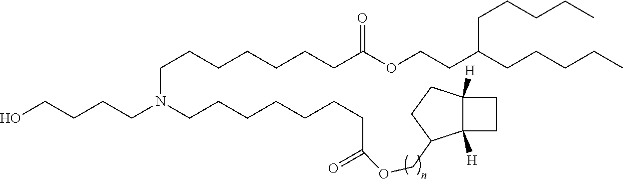 |
| CT-35 | 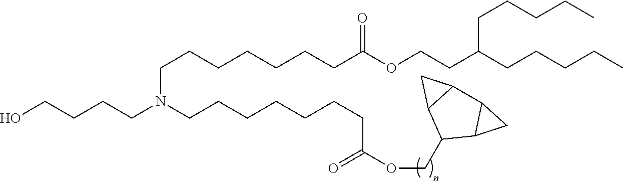 |
| CT-36 | 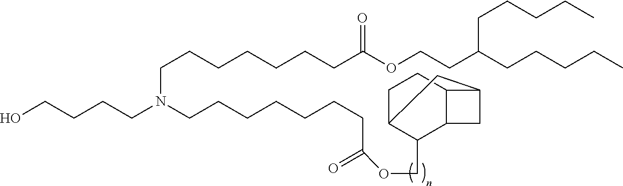 |
| CT-37 | 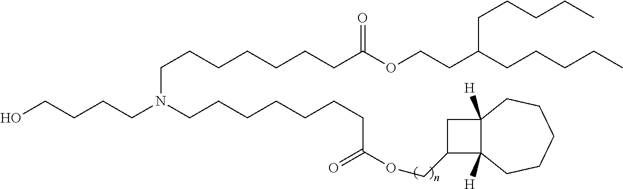 |
| CT-38 | 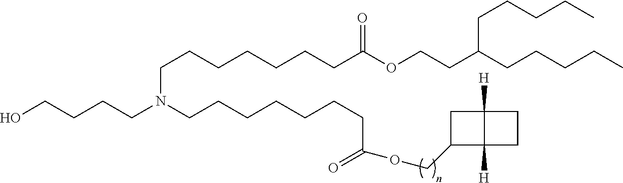 |
| CT-39 | 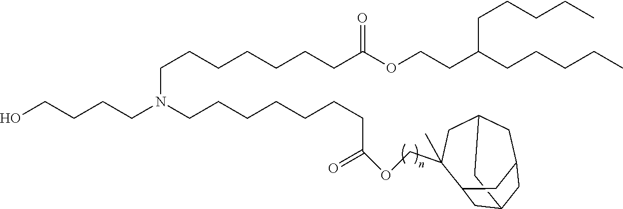 |
| CT-40 | 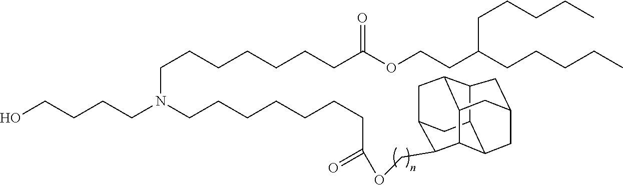 |
| CT-41 | 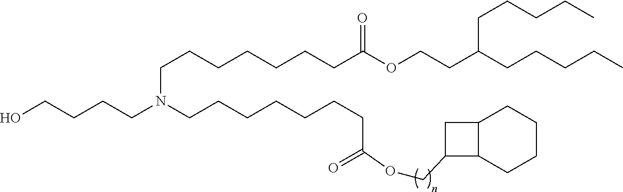 |
| CT-42 | 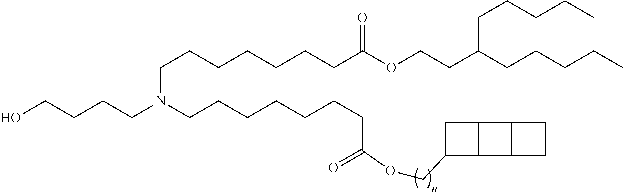 |
| CT-43 | 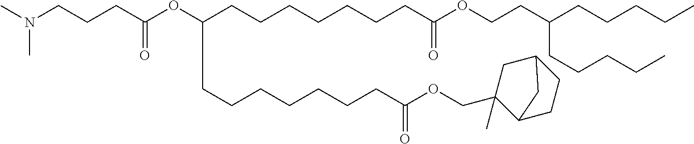 [SMILES: CCCCCC(CCCCC)CCOC(=O)CCCCCCCC(CCCCCCCCC(=O)OCC1(C)CC2CCC1C2)OC(=O)CCCN(C)C] |
| CT-44 | 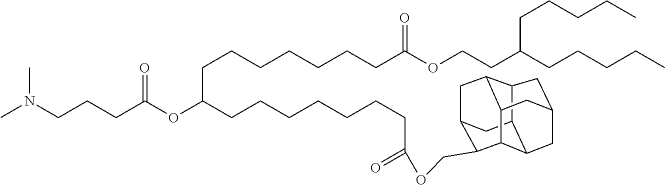 [SMILES: CCCCCC(CCCCC)CCOC(=O)CCCCCCCC(CCCCCCCC(=O)OCC1C2CC3C4CC5CC3C1C(C5)C4C2)OC(=O)CCCN(C)C] |
| CT-45 | 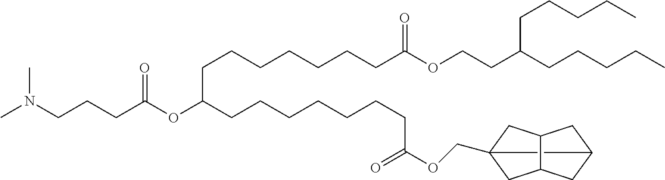 [SMILES: CCCCCC(CCCCC)CCOC(=O)CCCCCCCC(CCCCCCCC(=O)OCC12CC3CC1CC3C2)OC(=O)CCCN(C)C] |

where n is selected from integer 0-6. In some embodiments, n is 1.

### Series “AX”

Described below are a number of exemplary ionizable lipids of the present disclosure.

### Formula (AX)

The present disclosure, in some embodiments, provides compounds of Formula (AX″″)

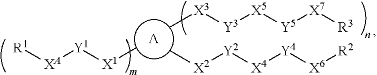

or a pharmaceutically acceptable salt thereof, wherein:
A is selected from an optionally substituted bridged carbocyclic bicycle, bridged carbocyclic multicycle, bridged heterocyclic bicycle, or bridged heterocyclic multicycle;
n is an integer selected from 0, 1 and 2;
m is an integer selected from 1 and 2, such that n plus n is less than or equal to 3;
R is selected from —OH, —OAc, —NR₂, —N(R)R¹¹,

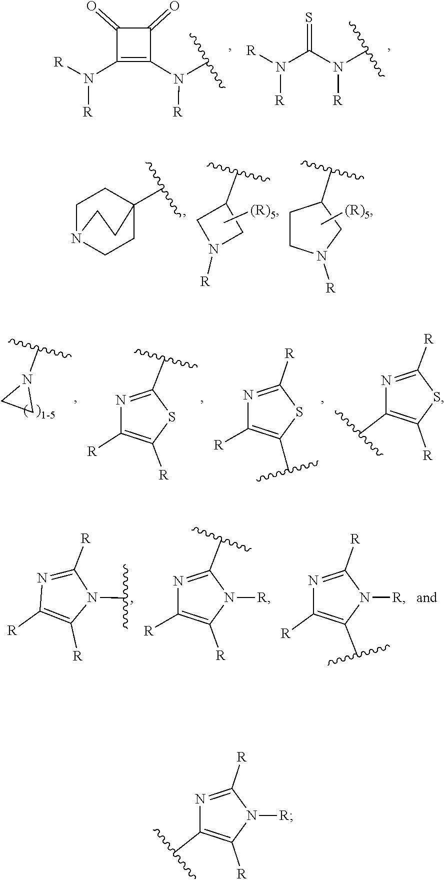

each R is independently —H or C1-C₆ aliphatic;
each R¹¹ is C₁-C₆ aliphatic-OH;
each X¹ and X^{A} is independently a bond or optionally substituted C₁-C₆ aliphatic;
each Y¹ is independently selected from

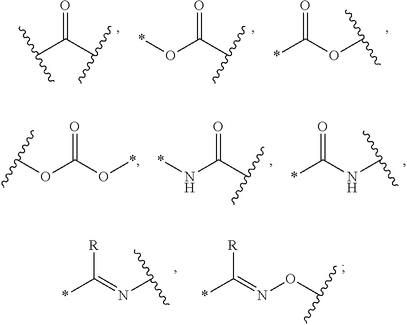

and a bond; wherein the bond marked with an “*” is attached to X¹;
each X and X³ is independently a bond or optionally substituted C₁-C₁₂ aliphatic;
each Y² and Y is independently selected from

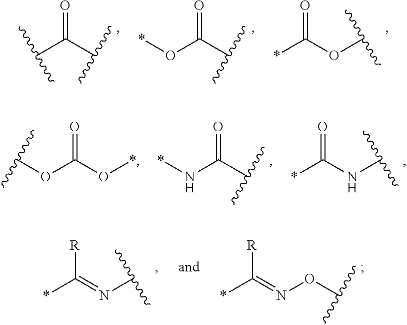

wherein the bond marked with an “*” is attached to X² or X³.
each X⁴ and X⁵ is independently a bond or optionally substituted C₁-C₆ aliphatic;
each Y⁴ and Y¹ is independently selected from a bond,

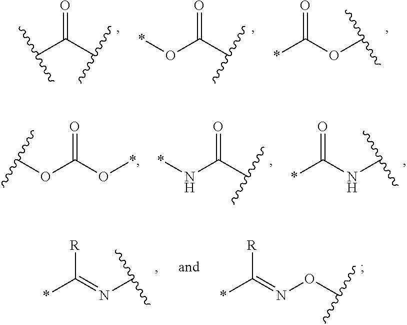

wherein the bond marked with an “*” is attached to X⁴ or X¹;
each X⁶ and X⁷ is independently a bond or optionally substituted C₁-C₆ aliphatic;
R² is —CH(OR⁶)(OR⁷), —CH(SR⁶)(SR⁷), —CH(R⁶)(R⁷), —CF(R⁶)(R⁷), —R¹⁰, optionally substituted C₅-C₁₈ aliphatic, or optionally substituted C₁-C₁₄ aliphatic-R¹⁰, wherein one or more methylene linkages of R$ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—; each R³ is independently —CH(OR⁸)(OR⁹), —CH(SR)(SR⁸), —CH(R⁸)(R⁹), —CF(R⁸)(R⁹), —R¹¹, optionally substituted C₅-C₁₈ aliphatic, or optionally substituted C₁-C₁₄ aliphatic: -R¹¹ wherein one or more methylene linkages of R³ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O
R⁶ and R⁷ are each independently optionally substituted —C₁-C₁₄ aliphatic, —R¹⁰, or optionally substituted —C₁-C₁₄ aliphatic-R₁; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁸ and R⁹ are each independently optionally substituted —C₁-C₁₄ aliphatic, —R¹¹, or optionally substituted —C₁-C₁ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—; and
each R¹⁰ and R¹¹ is independently an optionally substituted cylic, bicyclic, bridged bicyclic, multicyclic, or bridged multicyclic C₄-C₁₄ cycloalkyl or optionally substituted cylic, bicyclic, bridged bicyclic, multicyclic, or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl;
wherein one or more of X¹, X^{A}, X², X³, X⁴, X⁵, X⁶, X⁷, R² and R³ is optionally and independently substituted with one or more substituents selected from —F, —Cl, —Br and —I.

The present disclosure, in some embodiments, provides compounds of Formula (AX)

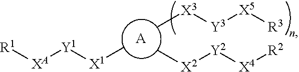

or a pharmaceutically acceptable salt thereof, wherein:
A is selected from an optionally substituted bridged carbocyclic or heterocyclic core selected from the group consisting of:

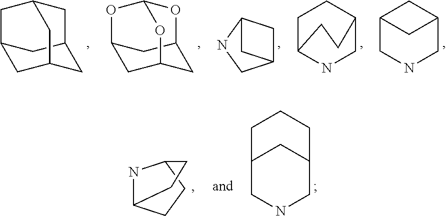

n is and integer selected from 1 or 2;
R¹ is selected from the group consisting of —OH, —OAc, —NR₂,

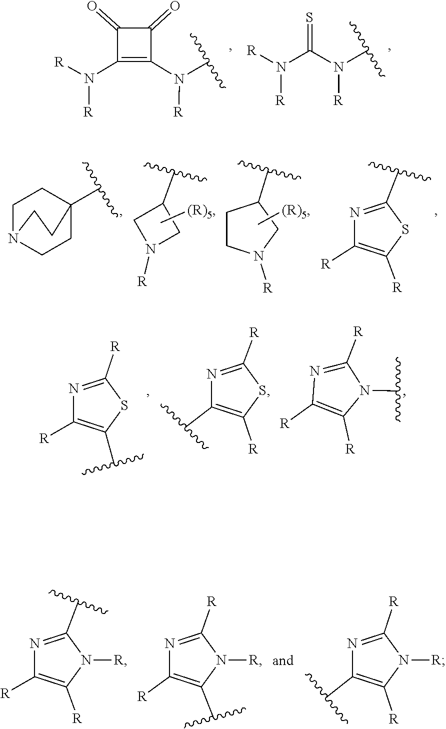

each R is independently —H or C₁-C₆ aliphatic;
X¹ and X^{A} are each independently a bond or optionally substituted C₁-C₆ aliphatic;
Y¹ is selected from the group consisting of

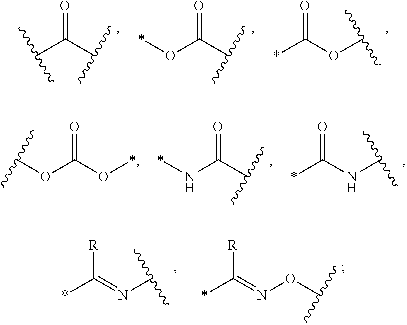

and bond; wherein the bond marked with an “*” is attached to X¹;
each X² and X³ is independently a bond or optionally substituted C₁-C₁₂ aliphatic;
each Y² and Y³ is independently selected from the group consisting of

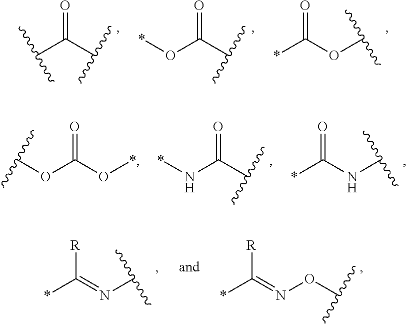

wherein the bond marked with an “*” is attached to X² or X³.
each X⁴ and X⁵ is independently optionally substituted C₁-C₆ aliphatic;
R² is —CH(OR⁶)(OR⁷), —CH(SR⁶)(SR⁷), —CH(R⁶)(R⁷), —R¹⁰, optionally substituted C₅-C₁₅ aliphatic, or optionally substituted C₁-C₁₄ aliphatic-R¹⁰, wherein one or more methylene linkages of R² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R³ is independently —CH(OR⁸)(OR⁹), —CH(SR⁸)(SR⁹), —CH(R⁸)(R⁹), —R¹¹, optionally substituted C₅-C₁₈ aliphatic, or optionally substituted C₁-C₁₄ aliphatic-R¹¹, wherein one or more methylene linkages of R³ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁶ and R⁷ are each independently optionally substituted —C₁-C₁₄ aliphatic, —R¹⁰, or optionally substituted —C₁-C₁₄ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁸ and R⁹ are each independently optionally substituted —C₁-C₁₄ aliphatic, —R¹¹, or optionally substituted —C₁-C₁₄ aliphatic-R¹¹; wherein one or more methylene linkages of R⁸ and R⁹ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R¹⁰ and R¹¹ is independently an optionally substituted cylic, bicyclic, bridged bicyclic, multicyclic or bridged multicyclic C₄-C₁₄ cycloalkyl or optionally substituted cylic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

The present disclosure, in some embodiments, provides compounds of Formula (AX″)

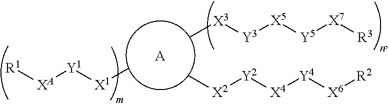

or a pharmaceutically acceptable salt thereof, wherein:
A is selected from an optionally substituted 4-14 membered bridged carbocyclic bicycle, bridged carbocyclic multicycle, bridged heterocyclic bicycle, or bridged heterocyclic multicycle;
each Y⁴ and Y⁵ is independently selected from the group consisting of a bond,

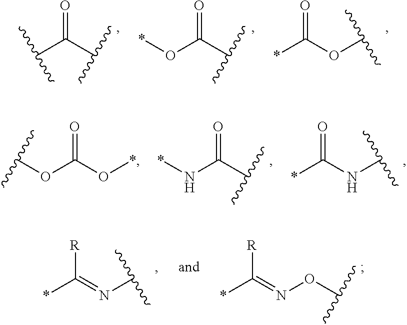

wherein the bond marked with an “*” is attached to X⁴ or X⁵;
each X⁶ and X⁷ is independently a bond or optionally substituted C₁-C₆ aliphatic;
n is and integer selected from 0, 1 or 2;
m is an integer selected from 1 or 2, such that m plus n is less than or equal to 3; and the remaining substituents and variables are as defined in Formula (AX).

### Additional Formulae

The present disclosure, in some embodiments, provides compounds of any one of the Formulae below:

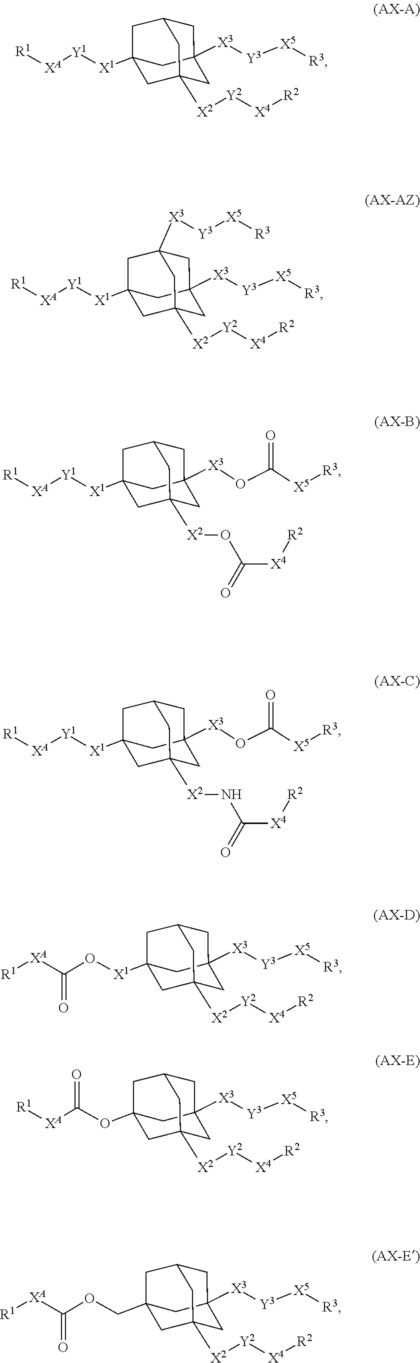

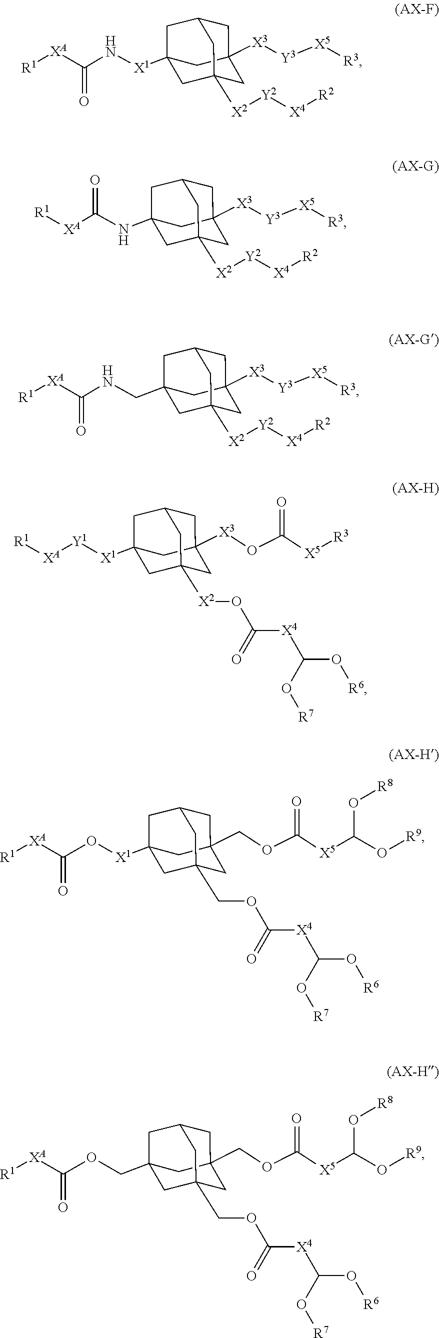

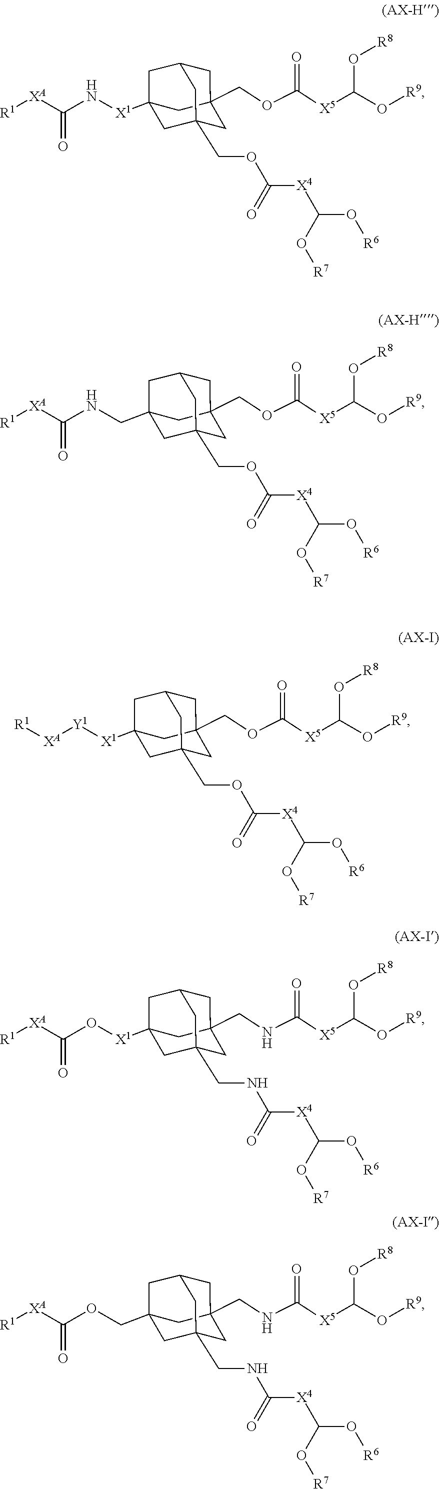

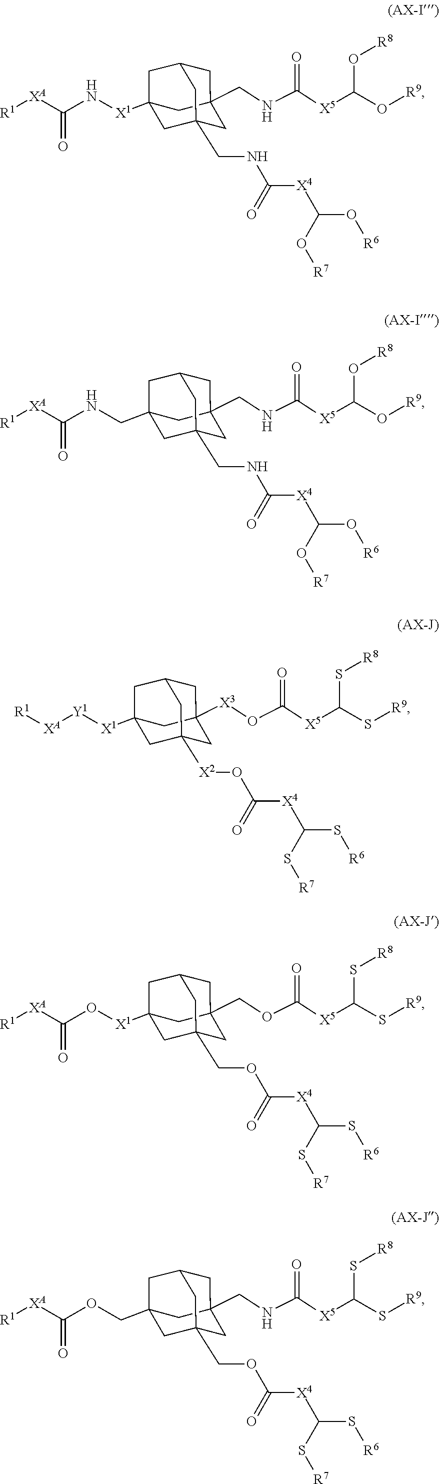

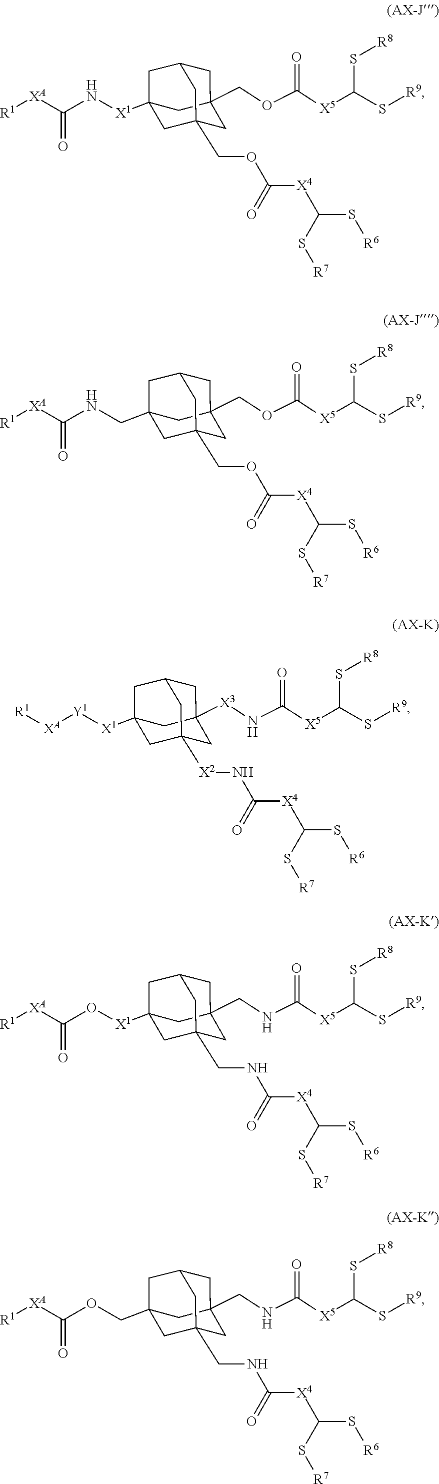

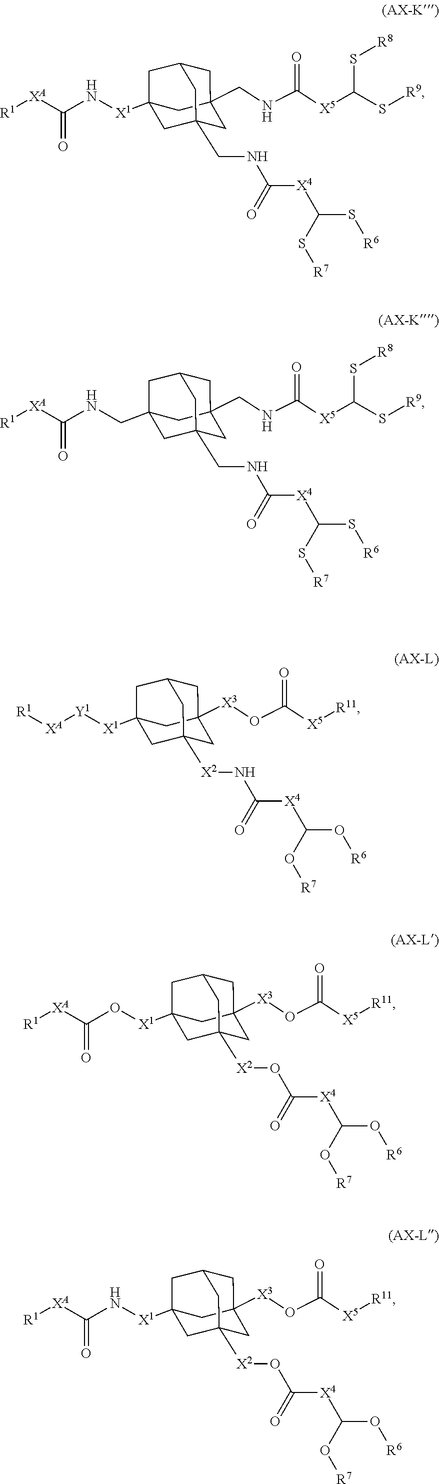

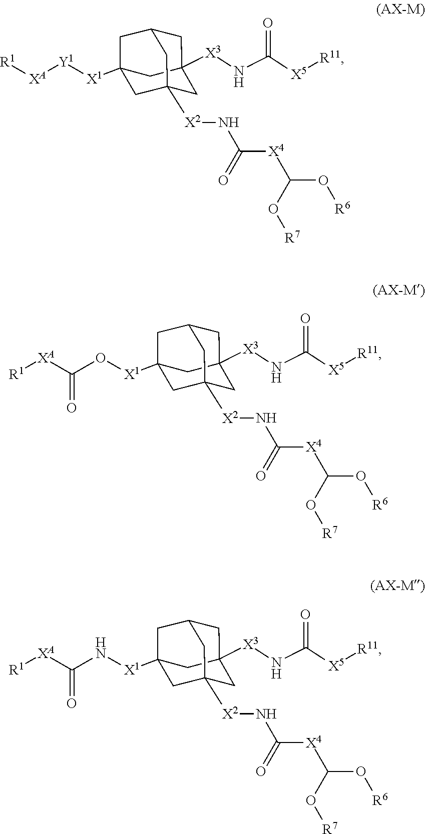

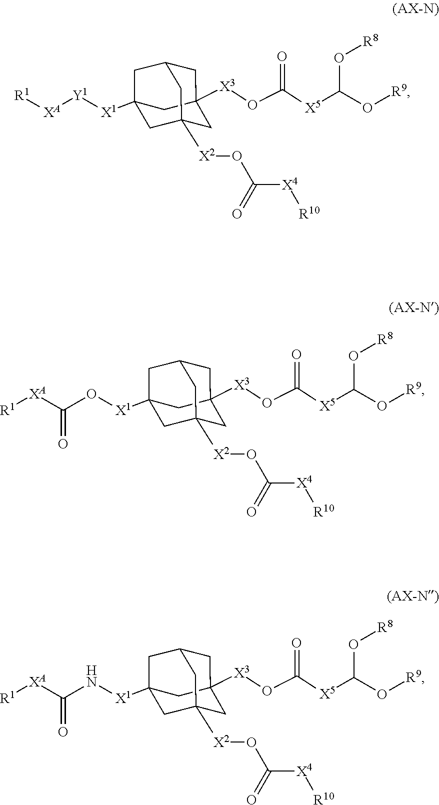

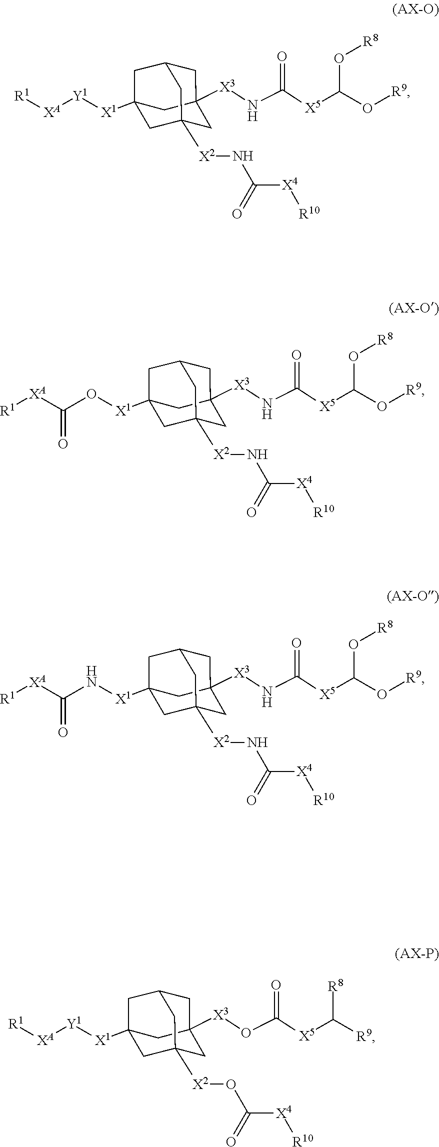

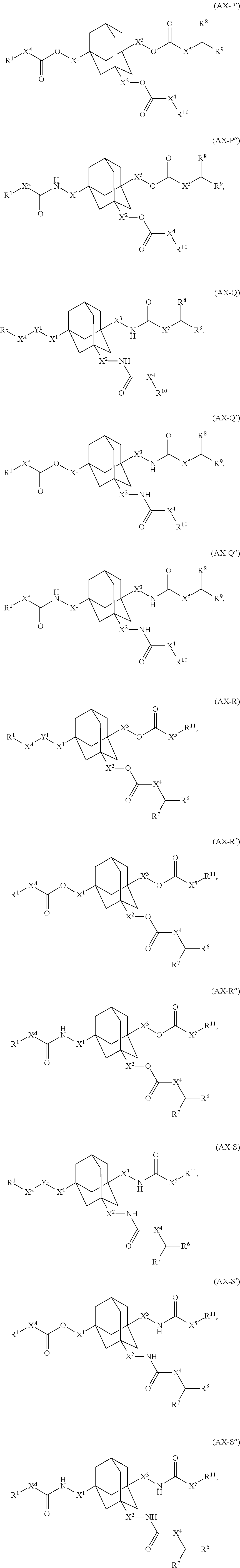

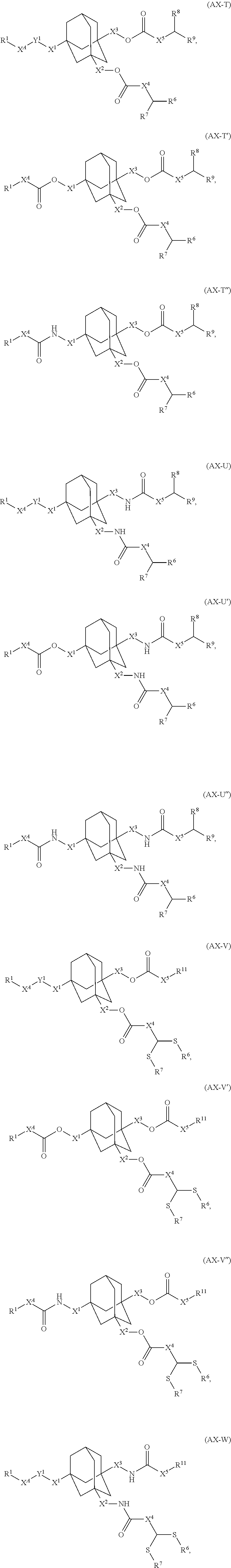

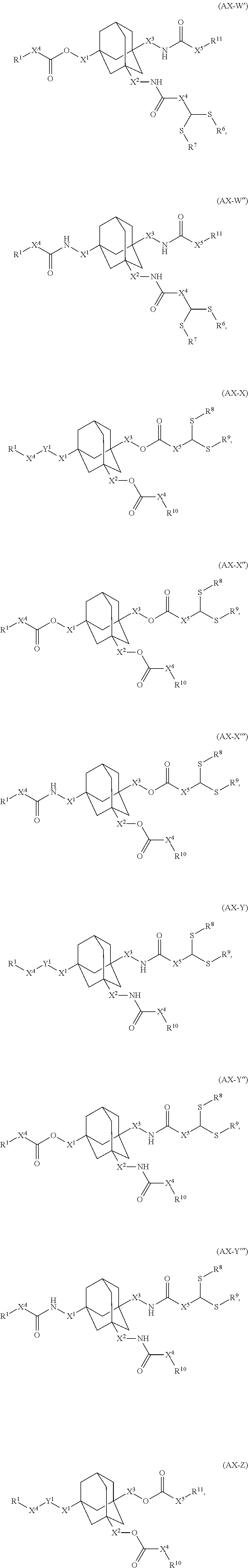

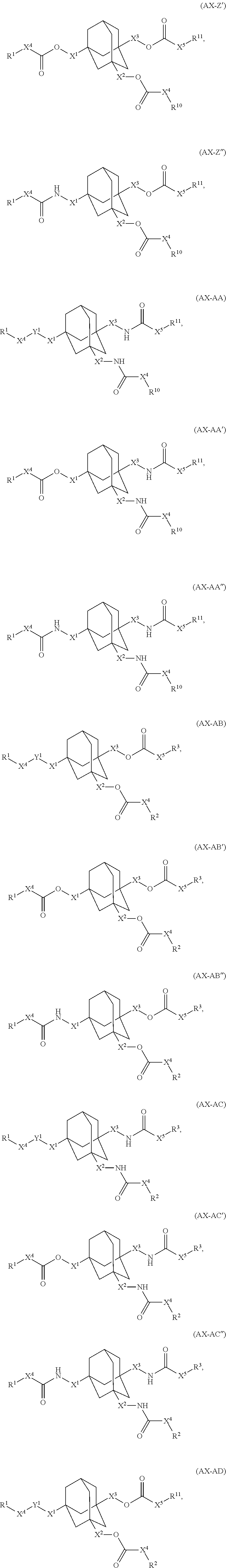

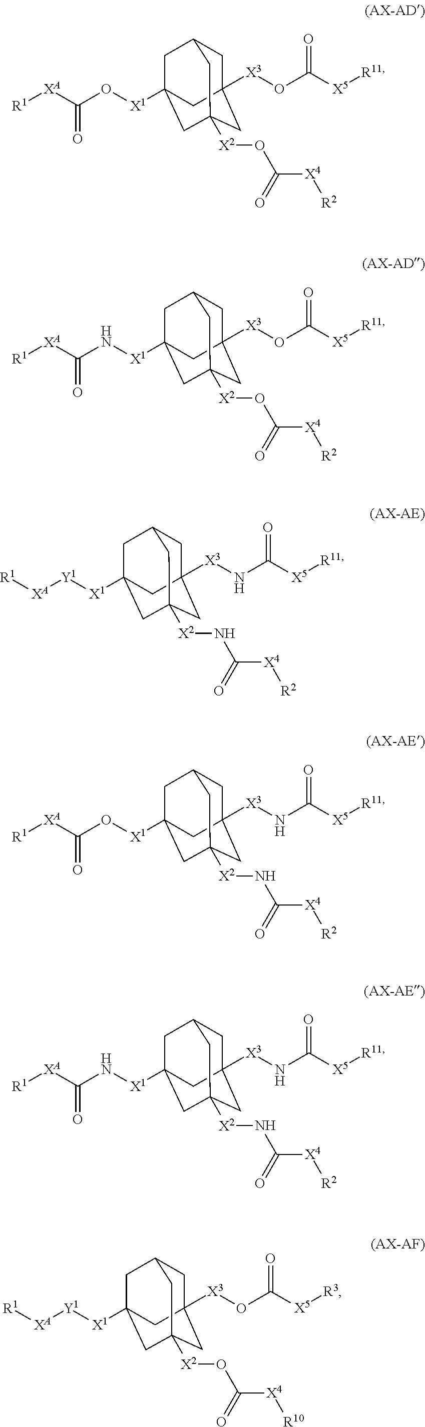

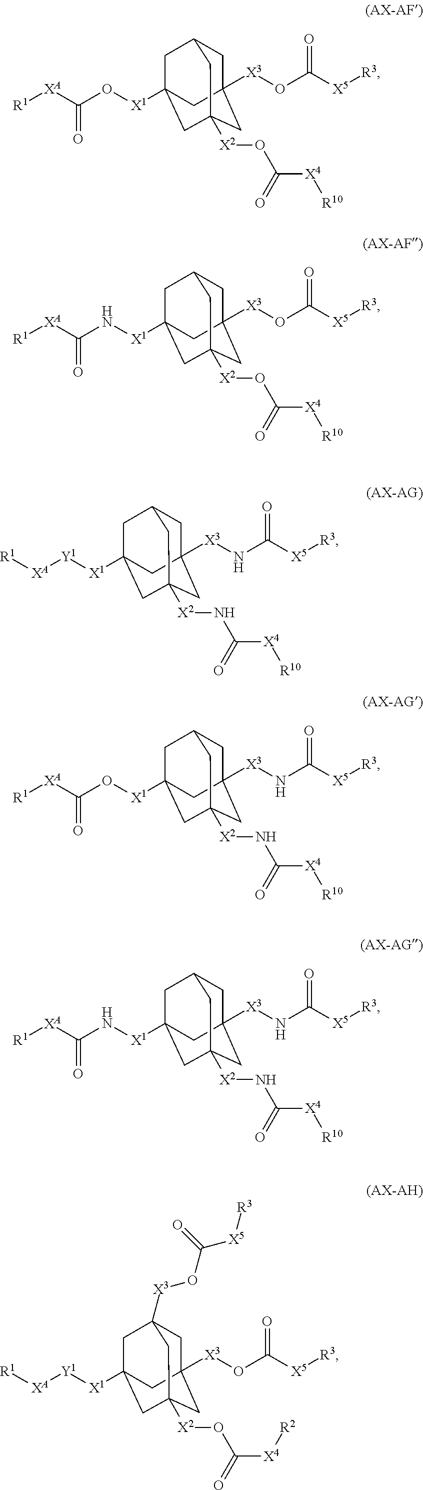

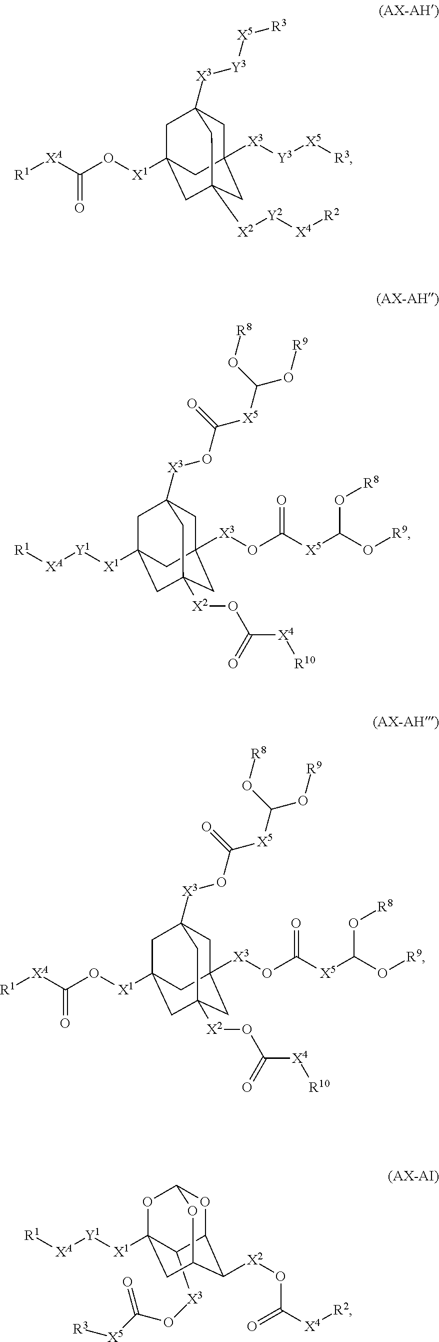

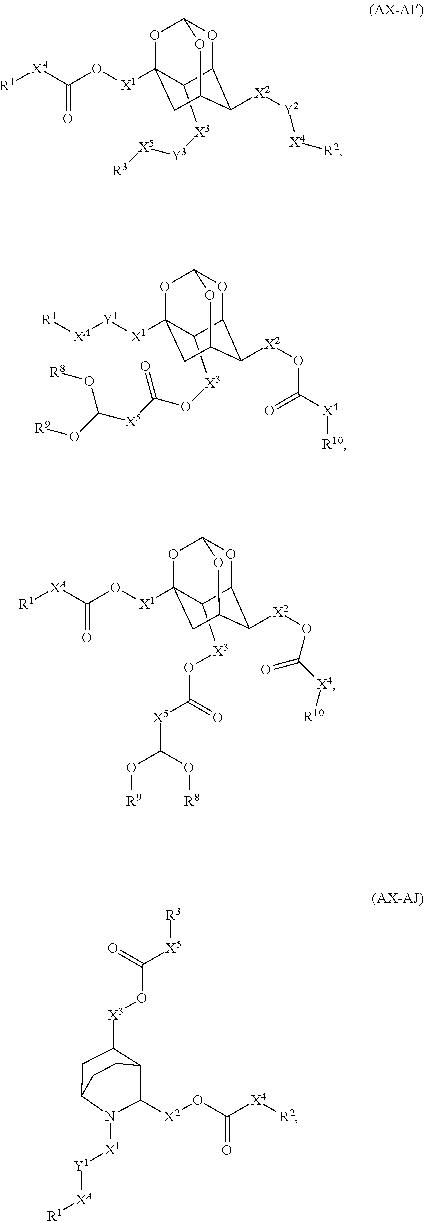

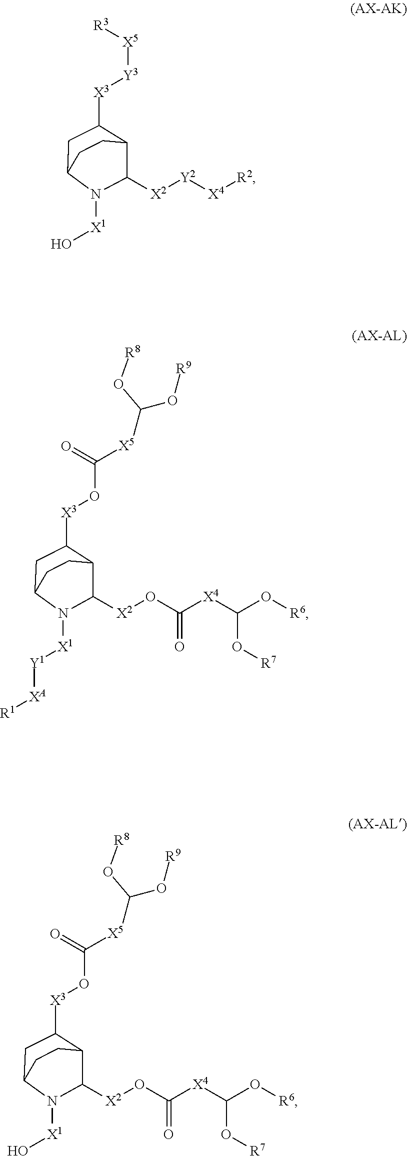

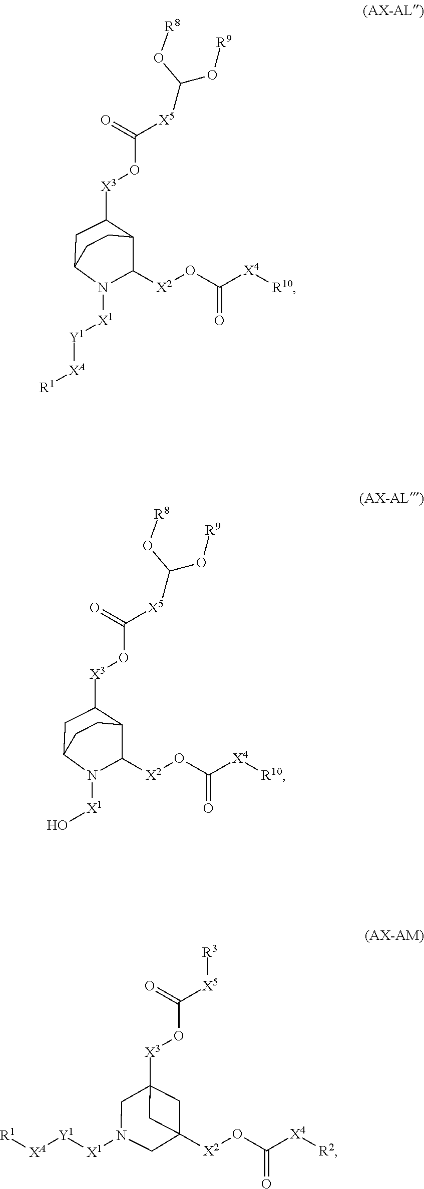

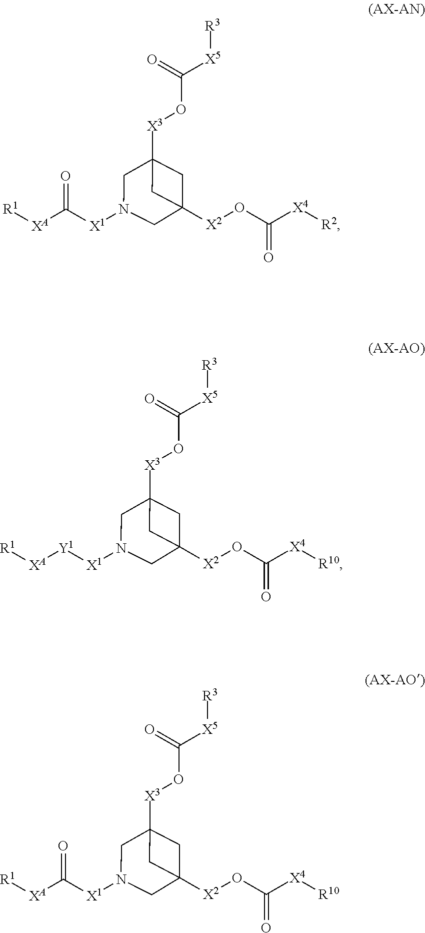

or a pharmaceutically acceptable salt thereof, wherein R¹, R, X¹, X^{A}, X², X³, X⁴, X⁵, Y¹, Y², Y³, R³, R⁶, R⁷, R⁸, R⁹, R¹⁰, and R¹¹ are as described in Formula (AX) or as otherwise described in any embodiments below.

### A

As disclosed in Formula (AX), in certain embodiments, A is

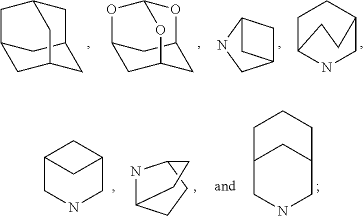

In certain embodiments, A is

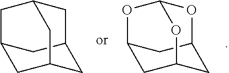 [SMILES: C1C2CC3CC1CC(C2)C3 ; C1C2CC3CC1OC(O2)O3]

In certain embodiments, A is a multivalent adamantyl core,

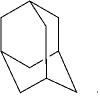

In certain embodiments, A is

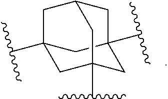 [SMILES: *C12CC3CC(*)(C1)CC(*)(C3)C2]

In certain embodiments, A is

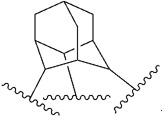 [SMILES: *C1C2CC3CC1C(*)C(C3)C2*]

In certain embodiments, A is

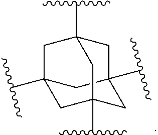 [SMILES: *C12CC3(*)CC(*)(C1)CC(*)(C2)C3]

In certain embodiments, A is a multivalent (1S,5S,7S)-2,4,10-trioxaadamantane core,

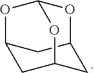

In certain embodiments, A is

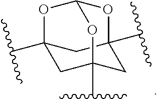 [SMILES: *C12CC3(*)CC(*)(C1)OC(O2)O3]

In certain embodiments, A is

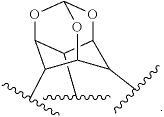 [SMILES: *C1C2OC3OC1C(*)C(O3)C2*]

In certain embodiments, A is

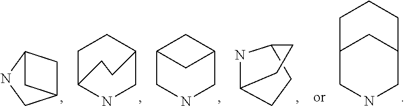

In certain embodiments, A is

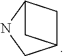

In certain embodiments, A is

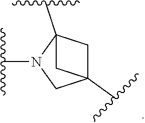 [SMILES: *N1CC2(*)CC1(*)C2]

In certain embodiments, A is

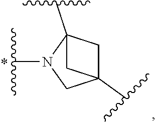 [SMILES: *N1CC2(*)CC1(*)C2]

wherein * is attached to X¹. In certain embodiments, A is

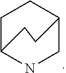

In certain embodiments, A is

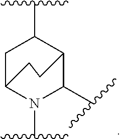 [SMILES: *C1CC2CCC1C(*)N2*]

In certain embodiments, A is

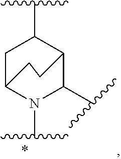 [SMILES: *C1CC2CCC1C(*)N2*]

wherein * is attached to X¹. In certain embodiments, A is

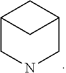

In certain embodiments, A is

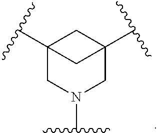 [SMILES: *N1CC2(*)CC(*)(C1)C2]

In certain embodiments, A is

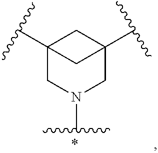 [SMILES: *N1CC2(*)CC(*)(C1)C2]

wherein * is attached to X¹. In certain embodiments, A is

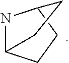

In certain embodiments, A is

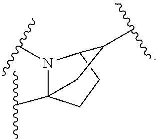 [SMILES: *C1CC2(*)CCC1N2*]

In certain embodiments, A is

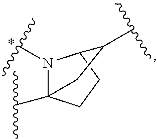 [SMILES: *C1CC2(*)CCC1N2*]

wherein * is attached to X¹. In certain embodiments, A is

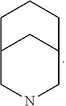

In certain embodiments, A is

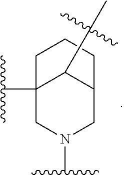 [SMILES: *C1C2CCCC1(*)CN(*)C2]

In certain embodiments, A is

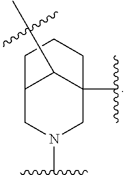 [SMILES: *C1C2CCCC1(*)CN(*)C2]

In certain embodiments, A is

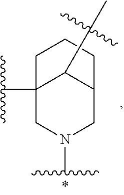 [SMILES: *C1C2CCCC1(*)CN(*)C2]

herein * is attached to X¹. In certain embodiments, A is

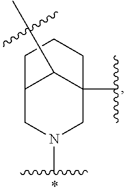 [SMILES: *C1C2CCCC1(*)CN(*)C2]

wherein * is attached to X¹.

In certain embodiments, A is selected from:

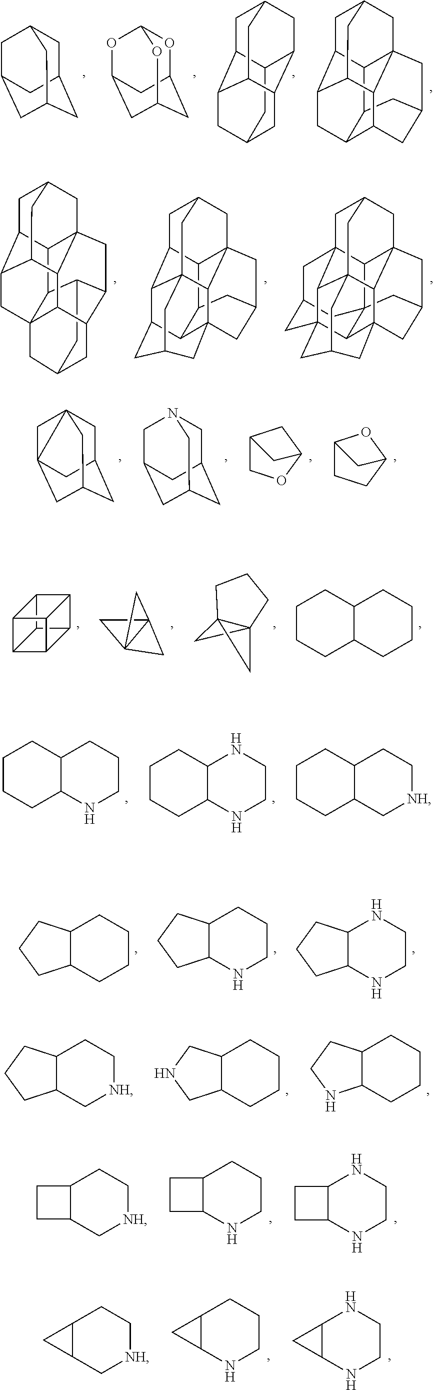

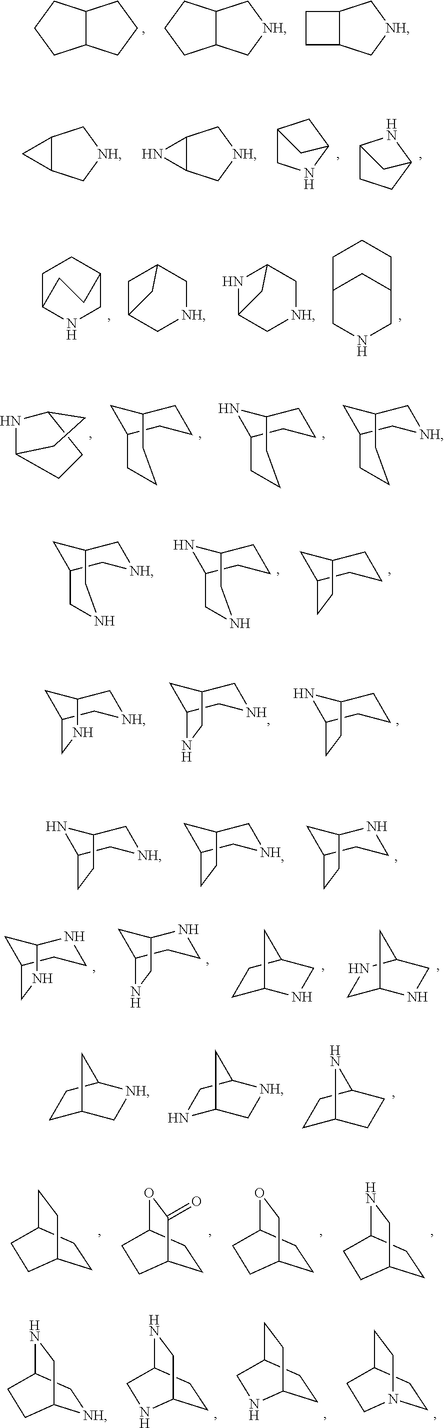

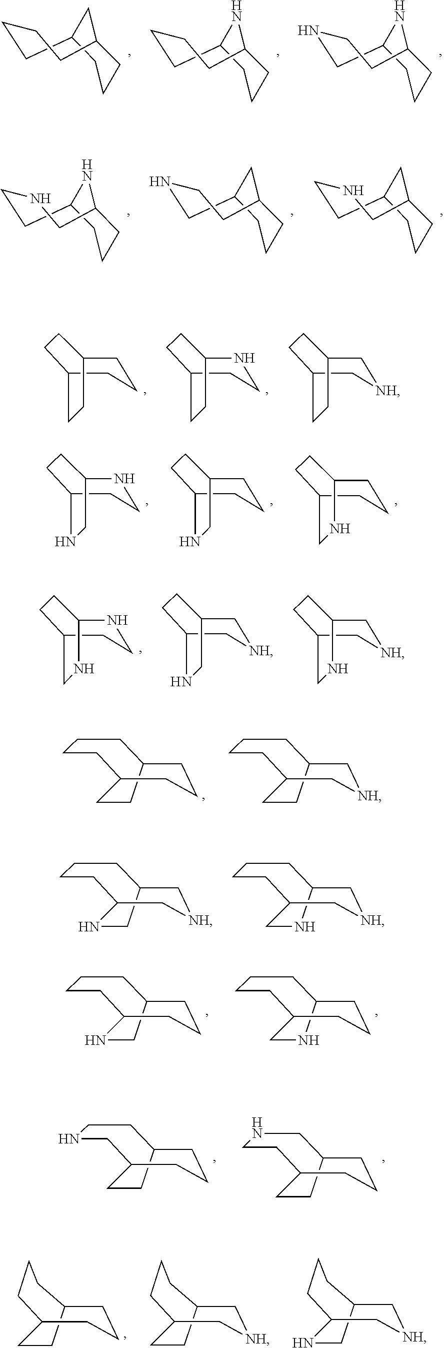

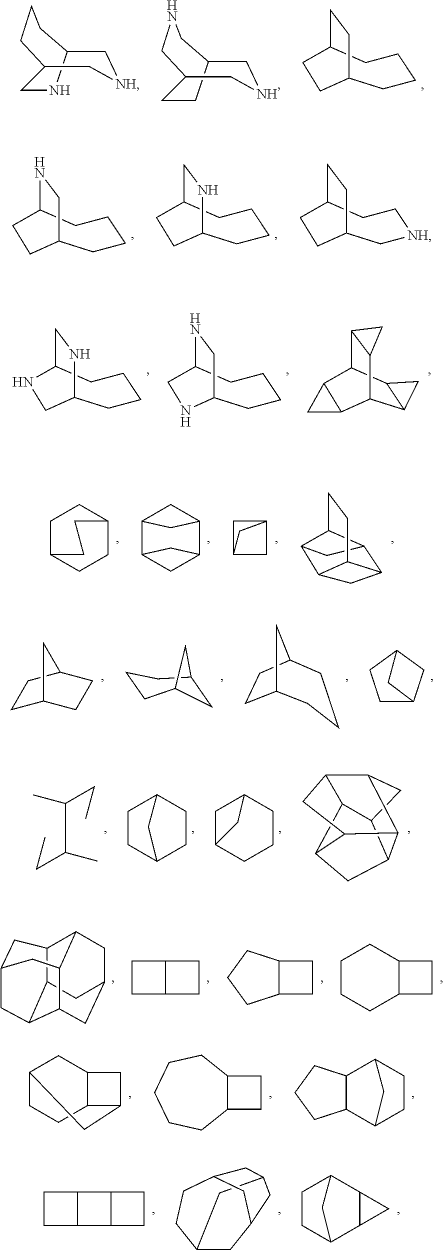

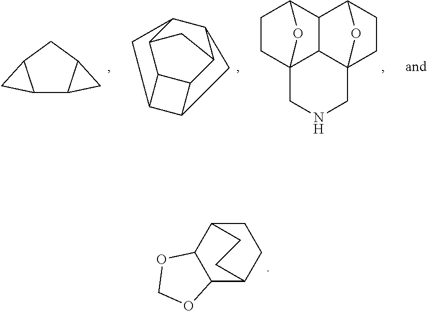

n and m

As disclosed in Formula (AX″″) in certain embodiments, n is an integer selected from 0, 1 or 2 and m is an integer selected from 1 or 2, such that m plus n is less than or equal to 3. In certain embodiments, m is 1 and n is 2. In certain embodiments, m is 2 and n is 1. In certain embodiments, m is 1 and n is 1. In certain embodiments, m is 1 and n is 0.

### Y¹

As disclosed in Formula (AX), in certain embodiments, Y¹ is

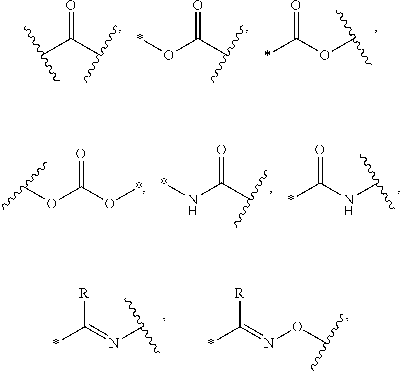

or bond; wherein the bond marked with an “*” is attached to X¹. In certain embodiments, Y¹ is

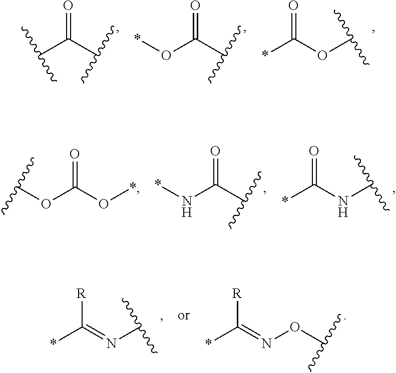

In certain embodiments, Y¹ is

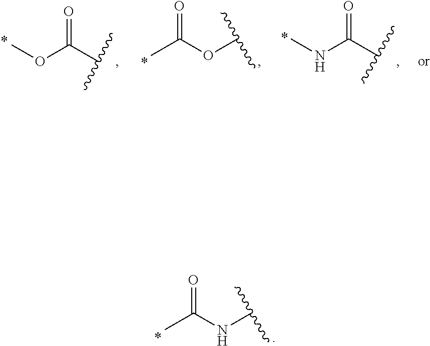

In certain embodiments, Y¹ is

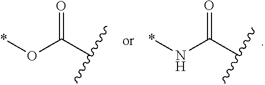 [SMILES: *OC(*)=O ; *NC(*)=O]

In certain embodiments, Y¹ is

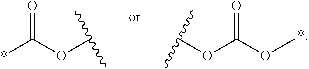

In certain embodiments, Y¹ is

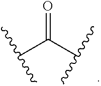 [SMILES: *C(*)=O]

In certain embodiments, Y¹ is

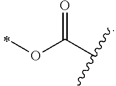

In certain embodiments, Y¹ is

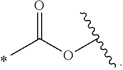 [SMILES: *OC(C)=O]

In certain embodiments, Y¹ is

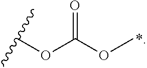

In certain embodiments, Y¹ is

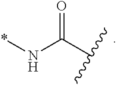 [SMILES: *NC(*)=O ; "."]

In certain embodiments, Y¹ is

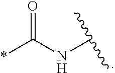 [SMILES: *NC(*)=O]

In certain embodiments, Y¹ is

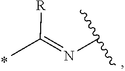

In certain embodiments, Y¹ is

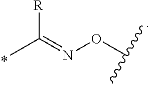

In certain embodiments, Y¹ is a bond.

### X¹

As disclosed in Formula (AX), in certain embodiments, X¹ is a bond or optionally substituted C₁-C₆ aliphatic. In certain embodiments, X¹ is a bond. In certain embodiments, X¹ is a bond or unsubstituted C₁-C₆ aliphatic. In certain embodiments, X¹ is unsubstituted C₁-C₆ aliphatic. In certain embodiments, X¹ is a bond or optionally substituted C₁-C₆ alkylene. In certain embodiments, X¹ is optionally substituted C₁-C₆ alkylene. In certain embodiments, X¹ is a bond or unsubstituted C₁-C₆ alkylene. In certain embodiments, X¹ is unsubstituted C₁-C₆ alkylene. In certain embodiments, X¹ is a bond or unsubstituted C₁-C₄ alkylene. In certain embodiments, X¹ is unsubstituted C₁-C₄ alkylene. In certain embodiments, X¹ is a bond or unsubstituted C₁-C₂ alkylene. In certain embodiments, X¹ is unsubstituted C₁-C₂ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### X^{A}

As disclosed in Formula (AX), in certain embodiments X^{A} is a bond or optionally substituted C₁-C₆ aliphatic. In certain embodiments, X^{A} is a bond. In certain embodiments, X^{A} is a bond or unsubstituted C₁-C₆ aliphatic. In certain embodiments, X^{A} is unsubstituted C₁-C₆ aliphatic. In certain embodiments, X^{A} is a bond or optionally substituted C₁-C₆ alkylene. In certain embodiments, X^{A} is optionally substituted C₁-C₆ alkylene. In certain embodiments, X^{A} is a bond or unsubstituted C₁-C₆ alkylene. In certain embodiments, X^{A} is unsubstituted C₁-C₆ alkylene. In certain embodiments, X^{A} is a bond or unsubstituted C₁-C₄ alkylene. In certain embodiments, X^{A} is unsubstituted C₁-C₄ alkylene. In certain embodiments, X^{A} is a bond or unsubstituted C₁-C₂ alkylene. In certain embodiments, X^{A} is unsubstituted C₁-C₂ alkylene. In certain embodiments, X^{A} is unsubstituted C₂-C₆ alkylene. In certain embodiments, X^{A} is optionally substituted methylene. In certain embodiments, R² is optionally substituted C₂ alkylene. In certain embodiments, X^{A} is optionally substituted C₃ alkylene. In certain embodiments, X^{A} is optionally substituted C₄ alkylene. In certain embodiments, X^{A} is optionally substituted C₅ alkylene. In certain embodiments, X^{A} is optionally substituted C₆ alkylene. In certain embodiments, X^{A} is —(CH₂)—. In certain embodiments, X^{A} is —(CH₂)₂—. In certain embodiments, X^{A} is —(CH₂)₃—. In certain embodiments, X^{A} is —(CH₂)₄—. In certain embodiments, X^{A} is —(CH₂)₅—. In certain embodiments, X^{A} is —(CH₂)₆—.

### R¹

As disclosed in Formula (AX), in certain embodiments R¹ is selected from —OH, —OAc, —NR₂

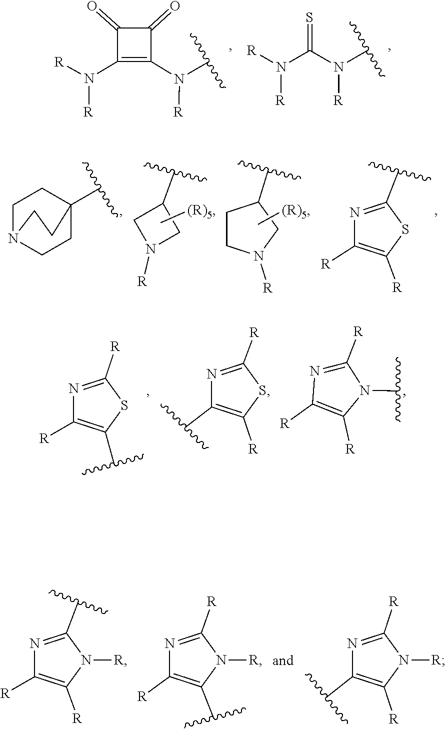

In certain embodiments, R¹ is OH, —NR₂, or

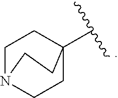 [SMILES: *C12CCN(CC1)CC2]

In certain embodiments, R¹ is —NR₂ or

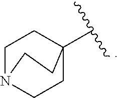 [SMILES: *C12CCN(CC1)CC2]

In certain embodiments, R¹ is —NMe₂, —NEt₂, or

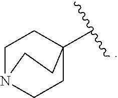 [SMILES: *C12CCN(CC1)CC2]

In certain embodiments, R¹ is —OH. In certain embodiments, R¹ is —OAc. In certain embodiments, R¹ is —NR₂. In certain embodiments, R¹ is —NH₂. In certain embodiments, R¹ is —NMe₂. In certain embodiments, R¹ is —NEt₂. In certain embodiments, R¹ is

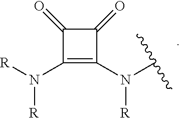

In certain embodiments, R¹ is

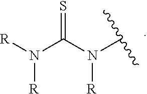

In certain embodiments, R¹ is

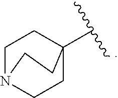 [SMILES: *C12CCN(CC1)CC2]

In certain embodiments, R¹ is

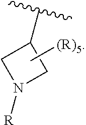

In certain embodiments, R¹ is

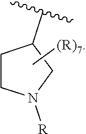

In certain embodiments, R¹ is

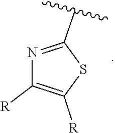

In certain embodiments, R¹ is

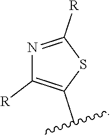

In certain embodiments, R¹ is

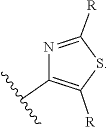

In certain embodiments, R¹ is

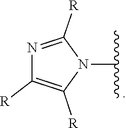

In certain embodiments, R¹ is

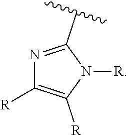

In certain embodiments, R¹ is

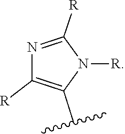

In certain embodiments, R¹ is

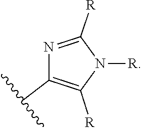

In certain embodiments, —X¹—Y¹—X^{A}—R¹ is

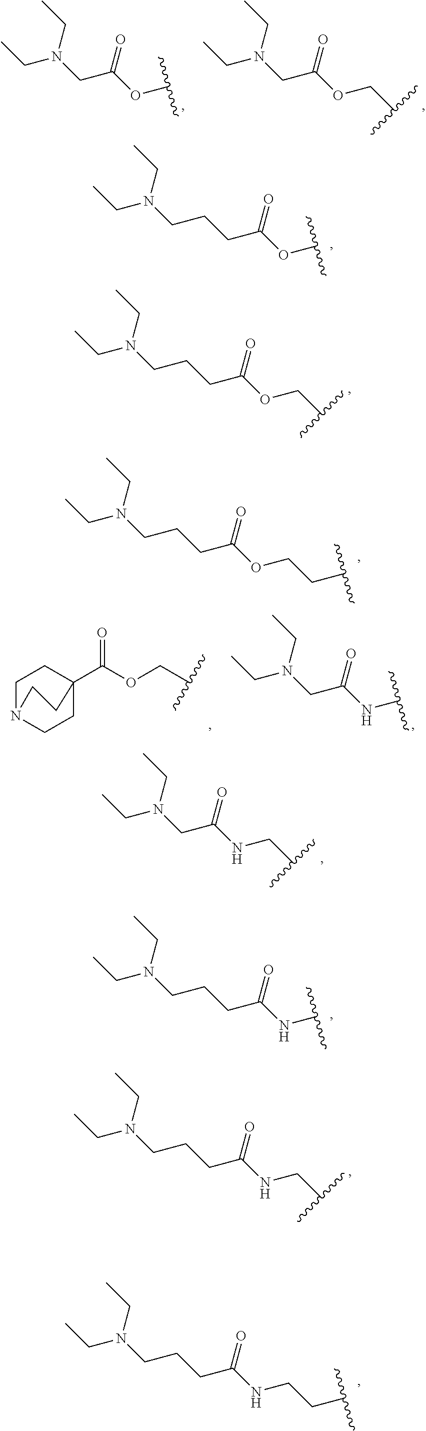

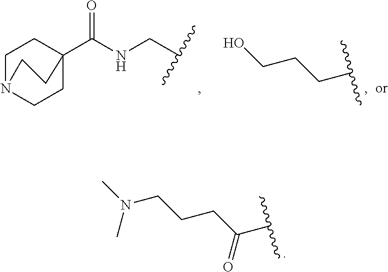

### R

As disclosed in Formula (AX), in certain embodiments R is —H or C₁-C₆ aliphatic. In certain embodiments, R is —H. In certain embodiments, R is C₁-C₆ aliphatic. In certain embodiments, R is or C₁-C₆ alkyl. In certain embodiments, R is C₁-C₄ alkyl. In certain embodiments, R is C₁-C₂ alkyl. In certain embodiments, R is unsubstituted C₁-C₆ alkyl. In certain embodiments, R is unsubstituted C₁-C₄alkyl. In certain embodiments, R is unsubstituted C₁-C₂ alkyl. In certain embodiments, R is methyl or ethyl.

X² and X³

As disclosed in Formula (AX), in certain embodiments, X² and X³ are each independently a bond or optionally substituted C₁-C₁₂ aliphatic. In certain embodiments, X² and X³ are the same. In certain embodiments, X² and X³ are different.

In certain embodiments, X² is a bond. In certain embodiments, X² is an optionally substituted C₁-C₁₂ alkylene. In certain embodiments, X² is an optionally substituted C₁-C₁₂ alkenylene. In certain embodiments, X² is an optionally substituted C₁-C₁₀ aliphatic. In certain embodiments, X² is an optionally substituted C₁-C₁₀ alkylene. In certain embodiments, X² is an optionally substituted C₁-C₁₀ alkenylene. In certain embodiments, X² is an optionally substituted C₁-C₈ aliphatic. In certain embodiments, X² is an optionally substituted C₁-C₈ alkylene. In certain embodiments, X² is an optionally substituted C₁-C₈ alkenylene. In certain embodiments, X² is an optionally substituted C₁-C₆ aliphatic. In certain embodiments, X² is an optionally substituted C₁-C₆ alkylene. In certain embodiments, X² is an optionally substituted C₁-C₆ alkenylene. In certain embodiments, X² is an optionally substituted C₂-C₁₂ aliphatic. In certain embodiments, X² is an optionally substituted C₂-C₁₂ alkylene. In certain embodiments, X² is an optionally substituted C₂-C₁₂ alkenylene. In certain embodiments, X² is an optionally substituted C₄-C₁₂ aliphatic. In certain embodiments, X² is an optionally substituted C₄-C₁₂ alkylene. In certain embodiments, X² is an optionally substituted C₄-C₁₂ alkenylene. In certain embodiments, X² is an optionally substituted C₄-C₁₀ aliphatic. In certain embodiments, X² is an optionally substituted C₄-C₁₀ alkylene. In certain embodiments, X² is an optionally substituted C₄-C₁₀ alkenylene. In certain embodiments, X² is an optionally substituted C₆-C₈ aliphatic. In certain embodiments, X² is an optionally substituted C₆-C₈ alkylene. In certain embodiments, X² is an optionally substituted C₆-C₈ alkenylene. In certain embodiments, X² is a bond or an optionally substituted C₁-C₄ aliphatic. In certain embodiments, X² is a bond or an optionally substituted C₁-C₄ alkylene. In certain embodiments, X² is a bond or an unsubstituted C₁-C₄ aliphatic. In certain embodiments, X² is a bond or an unsubstituted C₁-C₄ alkylene. In certain embodiments, X² is a bond or an optionally substituted C₁-C₂ aliphatic. In certain embodiments, X² is a bond or an optionally substituted C₁-C₂ alkylene. In certain embodiments, X² is a bond or an unsubstituted C₁-C₂ aliphatic. In certain embodiments, X² is a bond or an unsubstituted C₁-C₂ alkylene. In certain embodiments, X² is —(CH₂)—. In certain embodiments, X² is —(CH₂)₂—. In certain embodiments, X² is —(CH₂)₃—. In certain embodiments, X² is —(CH₂)₄—. In certain embodiments, X² is —(CH₂)₅—. In certain embodiments, X² is —(CH₂)₆—. In certain embodiments, X² is —(CH₂)₇—. In certain embodiments, X² is —(CH₂)₈—. In certain embodiments, X² is —(CH₂)₉—. In certain embodiments, X² is —(CH₂)₁₀—.

In certain embodiments, X³ is a bond. In certain embodiments, X³ is an optionally substituted C₁-C₁₂ alkylene. In certain embodiments, X³ is an optionally substituted C₁-C₁₂ alkenylene. In certain embodiments, X³ is an optionally substituted C₁-C₁₀ aliphatic. In certain embodiments, X³ is an optionally substituted C₁-C₁₀ alkylene. In certain embodiments, X³ is an optionally substituted C₁-C₁₀ alkenylene. In certain embodiments, X³ is an optionally substituted C₁-C₈ aliphatic. In certain embodiments, X³ is an optionally substituted C₁-C₈ alkylene. In certain embodiments, X³ is an optionally substituted C₁-C₈ alkenylene. In certain embodiments, X³ is an optionally substituted C₁-C₆ aliphatic. In certain embodiments, X³ is an optionally substituted C₁-C₆ alkylene. In certain embodiments, X³ is an optionally substituted C₁-C₆ alkenylene. In certain embodiments, X³ is an optionally substituted C₂-C₁₂ aliphatic. In certain embodiments, X³ is an optionally substituted C₂-C₁₂ alkylene. In certain embodiments, X³ is an optionally substituted C₂-C₁₂ alkenylene. In certain embodiments, X³ is an optionally substituted C₄-C₁₂ aliphatic. In certain embodiments, X³ is an optionally substituted C₄-C₁₂ alkylene. In certain embodiments, X³ is an optionally substituted C₄-C₁₂ alkenylene. In certain embodiments, X³ is an optionally substituted C₄-C₁₀ aliphatic. In certain embodiments, X³ is an optionally substituted C₄-C₁₀ alkylene. In certain embodiments, X³ is an optionally substituted C₄-C₁₀ alkenylene. In certain embodiments, X³ is an optionally substituted C₆-C₈ aliphatic. In certain embodiments, X³ is an optionally substituted C₆-C₈ alkylene. In certain embodiments, X³ is an optionally substituted C₆-C₈ alkenylene. In certain embodiments, X³ is a bond or an optionally substituted C₁-C₄ aliphatic. In certain embodiments, X³ is a bond or an optionally substituted C₁-C₄ alkylene. In certain embodiments, X³ is a bond or an unsubstituted C₁-C₄ aliphatic. In certain embodiments, X³ is a bond or an unsubstituted C₁-C₄ alkylene. In certain embodiments, X³ is a bond or an optionally substituted C₁-C₂ aliphatic. In certain embodiments, X³ is a bond or an optionally substituted C₁-C₂ alkylene. In certain embodiments, X³ is a bond or an unsubstituted C₁-C₂ aliphatic. In certain embodiments, X³ is a bond or an unsubstituted C₁-C₂ alkylene. In certain embodiments, X³ is —(CH₂)—. In certain embodiments, X³ is —(CH₂)₂—. In certain embodiments, X³ is —(CH₂)₃—. In certain embodiments, X³ is —(CH₂)₄—. In certain embodiments, X³ is —(CH₂)₅—. In certain embodiments, X³ is —(CH₂)₆—. In certain embodiments, X³ is —(CH₂)₇—. In certain embodiments, X³ is —(CH₂)₈—. In certain embodiments, X³ is —(CH₂)₉—. In certain embodiments, X³ is —(CH₂)₁₀—.

In certain embodiments, X² and X³ are each independently a bond or an optionally substituted C₁-C₄ aliphatic. In certain embodiments, X² and X³ are each independently a bond or an optionally substituted C₁-C₄ alkylene. In certain embodiments, X² and X³ are each independently a bond or an unsubstituted C₁-C₄ aliphatic. In certain embodiments, X² and X³ are each independently a bond or an unsubstituted C₁-C₄ alkylene. In certain embodiments, X² and X³ are each independently a bond or an optionally substituted C₁-C₂ aliphatic. In certain embodiments, X² and X³ are each independently a bond or an optionally substituted C₁-C₂ alkylene. In certain embodiments, X² and X³ are each independently a bond or an unsubstituted C₁-C₂ aliphatic. In certain embodiments, X² and X³ are each independently a bond or an unsubstituted C₁-C₂ alkylene.

In certain embodiments, X² and X³ are both a bond or an optionally substituted C₁-C₄ aliphatic. In certain embodiments, X² and X³ are both a bond or an optionally substituted C₁-C₄ alkylene. In certain embodiments, X² and X³ are both a bond or an unsubstituted C₁-C₄ aliphatic. In certain embodiments, X² and X³ are both a bond or an unsubstituted C₁-C₄ alkylene. In certain embodiments, X² and X³ are both a bond or an optionally substituted C₁-C₂ aliphatic. In certain embodiments, X² and X³ are both a bond or an optionally substituted C₁-C₂ alkylene. In certain embodiments, X² and X³ are both a bond or an unsubstituted C₁-C₂ aliphatic. In certain embodiments, X² and X³ are both a bond or an unsubstituted C₁-C₂ alkylene.

In certain embodiments, X² and X³ are both bonds. In certain embodiments, X² and X³ are both —(CH₂)—. In certain embodiments, X² and X³ are both —(CH₂)₂—. In certain embodiments, X² and X³ are both —(CH₂)₃—.

Y² and Y³

As disclosed in Formula (AX), in certain embodiments, Y² and Y³ are each independently

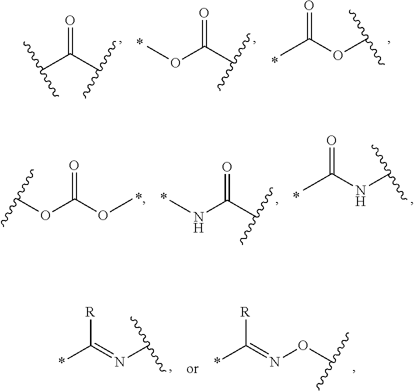

wherein the bond marked with an “*” is attached to X² for Y² or X³ for Y³. In certain embodiments, Y² and Y³ are the same. In certain embodiments, Y² and Y³ are different.

In certain embodiments, Y² and Y³ are each independently

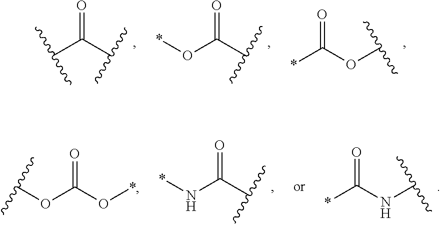

In certain embodiments, Y² and Y³ are each independently

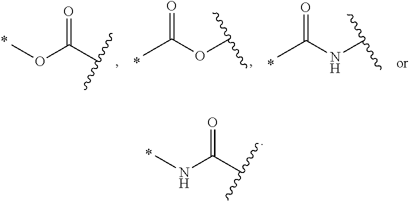

In certain embodiments, Y² and Y³ are each independently

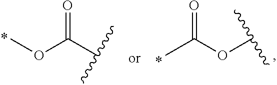

In certain embodiments, Y² and Y³ are each independently

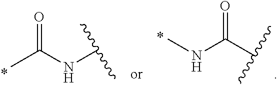

In certain embodiments, Y² and Y³ are each independently

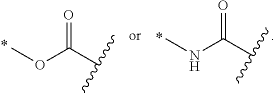

In certain embodiments, Y²

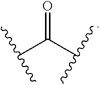 [SMILES: *C(*)=O]

In certain embodiments, Y² is

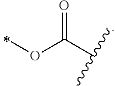

In certain embodiments, Y²

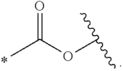 [SMILES: *OC(C)=O]

In certain embodiments, Y² is

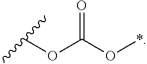

In certain embodiments, Y² is

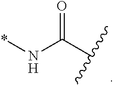 [SMILES: *NC(*)=O]

In certain embodiments, Y² is

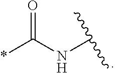 [SMILES: *NC(*)=O]

In certain embodiments, Y² is

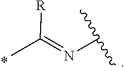

In certain embodiments, Y² is

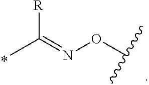

In certain embodiments, Y³ is

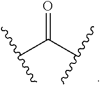 [SMILES: *C(*)=O]

In certain embodiments, Y³ is

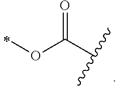

In certain embodiments, Y³ is

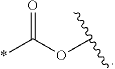 [SMILES: *OC(*)=O]

In certain embodiments, Y³

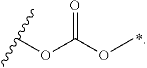

In certain embodiments, Y³ is

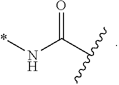 [SMILES: *NC(*)=O]

In certain embodiments, Y³ is

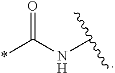 [SMILES: *NC(*)=O]

In certain embodiments, Y³ is

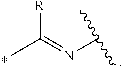

In certain embodiments, Y³ is

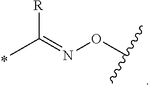

In certain embodiments, Y³ and Y² are both

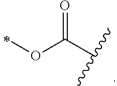

In certain embodiments, Y³ and Y² are both

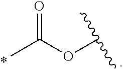 [SMILES: *OC(C)=O]

In certain embodiments, Y³ and Y² are both

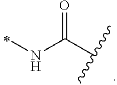

H In certain embodiments, Y³ and Y² are both

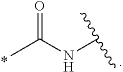 [SMILES: *NC(C)=O]

X⁴ and X⁵

As disclosed in Formula (AX″″), in certain embodiments, X⁴ and X⁵ are each independently a bond or optionally substituted C₁-C₆ aliphatic. As disclosed in Formula (AX), in certain embodiments, X⁴ and X⁵ are each independently optionally substituted C₁-C₆ aliphatic. In certain embodiments, X⁴ and X⁵ are the same. In certain embodiments, X⁴ and X⁵ are different.

In certain embodiments, X⁴ is a bond. In certain embodiments, X⁴ is an optionally substituted C₁-C₆ aliphatic. In certain embodiments, X⁴ is an optionally substituted C₁-C₆ alkylene. In certain embodiments, X⁴ is an optionally substituted C₁-C₆ alkenylene. In certain embodiments, X⁴ is an optionally substituted C₂-C₅ aliphatic. In certain embodiments, X⁴ is an optionally substituted C₂-C₅ alkylene. In certain embodiments, X⁴ is an optionally substituted C₂-C₅ alkenylene. In certain embodiments, X⁴ is an optionally substituted C₃-C₄ aliphatic. In certain embodiments, X⁴ is an optionally substituted C₃-C₄ alkylene. In certain embodiments, X⁴ is an optionally substituted C₃-C₄ alkenylene. In certain embodiments, X⁴ is —(CH₂)—. In certain embodiments, X⁴ is —(CH₂)₂—. In certain embodiments, X⁴ is —(CH₂)₃—. In certain embodiments, X⁴ is —(CH₂)₄—. In certain embodiments, X⁴ is —(CH₂)₅—. In certain embodiments, X⁴ is —(CH₂)₆—.

In certain embodiments, X⁵ is a bond. In certain embodiments, X⁵ is an optionally substituted C₁-C₆ aliphatic. In certain embodiments, X⁵ is an optionally substituted C₁-C₆ alkylene. In certain embodiments, X⁵ is an optionally substituted C₁-C₆ alkenylene. In certain embodiments, X⁵ is an optionally substituted C₂-C₅ aliphatic. In certain embodiments, X⁵ is an optionally substituted C₂-C₅ alkylene. In certain embodiments, X⁵ is an optionally substituted C₂-C₅ alkenylene. In certain embodiments, X⁵ is an optionally substituted C₃-C₄ aliphatic. In certain embodiments, X⁵ is an optionally substituted C₃-C₄ alkylene. In certain embodiments, X⁵ is an optionally substituted C₃-C₄ alkenylene. In certain embodiments, X⁵ is —(CH₂)—. In certain embodiments, X⁵ is —(CH₂)₂—. In certain embodiments, X⁵ is —(CH₂)₃—. In certain embodiments, X⁵ is —(CH₂)₄—. In certain embodiments, X⁵ is —(CH₂)₅—. In certain embodiments, X⁵ is —(CH₂)₆—.

In certain embodiments, X⁴ and X⁵ are each independently an optionally substituted C₁-C₄ aliphatic. In certain embodiments, X⁴ and X⁵ are each independently an optionally substituted C₁-C₄ alkylene. In certain embodiments, X⁴ and X⁵ are each independently an unsubstituted C₁-C₄ aliphatic. In certain embodiments, X⁴ and X⁵ are each independently an unsubstituted C₁-C₄ alkylene. In certain embodiments, X⁴ and X⁵ are each independently an optionally substituted C₁-C₂ aliphatic. In certain embodiments, X⁴ and X⁵ are each independently an optionally substituted C₁-C₂ alkylene. In certain embodiments, X⁴ and X⁵ are each independently an unsubstituted C₁-C₂ aliphatic. In certain embodiments, X⁴ and X⁵ are each independently an unsubstituted C₁-C₂ alkylene.

In certain embodiments, X⁴ and X⁵ are both an optionally substituted C₁-C₄ aliphatic. In certain embodiments, X⁴ and X⁵ are both an optionally substituted C₁-C₄ alkylene. In certain embodiments, X⁴ and X⁵ are both an unsubstituted C₁-C₄ aliphatic. In certain embodiments, X⁴ and X⁵ are both an unsubstituted C₁-C₄ alkylene. In certain embodiments, X⁴ and X⁵ are both an optionally substituted C₁-C₂ aliphatic. In certain embodiments, X⁴ and X⁵ are both an optionally substituted C₁-C₂ alkylene. In certain embodiments, X⁴ and X⁵ are both an unsubstituted C₁-C₂ aliphatic. In certain embodiments, X⁴ and X⁵ are both an unsubstituted C₁-C₂ alkylene.

In certain embodiments, X⁴ and X⁵ are both a bond. In certain embodiments, X⁴ and X⁵ are both —(CH₂)—. In certain embodiments, X⁴ and X⁵ are both —(CH₂)₂—. In certain embodiments, X⁴ and X⁵ are both —(CH₂)₃—. In certain embodiments, X⁴ is —(CH₂)₂— and X⁵ is —(CH₂)—. In certain embodiments, X⁴ and X⁵ are both —(CH₂)₄—. In certain embodiments, X⁴ and X⁵ are both —(CH₂)₅—. In certain embodiments, X⁴ and X⁵ are both —(CH₂)₆—.

In certain embodiments, X⁴ and X⁵ are each independently substituted with one or more substituents selected from —F, —Cl, —Br and —I. In certain embodiments, X⁴ and/or X⁵ are substituted with one or more —F. In certain embodiments, X⁴ and X⁵ are substituted with one or more —F on a carbon atom at a position selected from α-position and β-position from Y⁴ or Y⁵, respectively. In certain embodiments, X⁴ and/or X⁵ are substituted with one or more —F. In certain embodiments, X⁴ and X⁵ are substituted with one or more —F on a carbon atom at a position selected from α-position and β-position from Y² or Y³, respectively.

In certain embodiments, wherein the compound comprises two X⁵ (in other words, when n=2); each X⁵ is independently selected from any of the embodiments above, and need not be the same. In certain embodiments, wherein the compound comprises two X⁵, they are each the same.

Y⁴ and Y⁵

As disclosed in Formula (AX″″), in certain embodiments, Y⁴ and Y⁵ are each independently a bond,

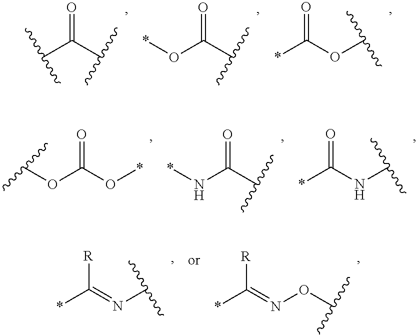

wherein the bond marked with an “*” is attached to X⁴ for Y⁴ or X⁵ for Y⁵. In certain embodiments, Y⁴ and Y⁵ are the same. In certain embodiments, Y⁴ and Y⁵ are different.

In certain embodiments, Y⁴ and Y⁵ are each independently

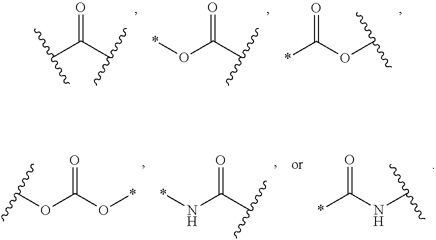

In certain embodiments, Y⁴ and Y⁵ are each independently

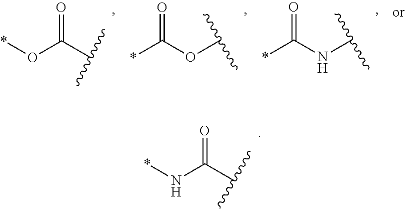

In certain embodiments, Y² and Y⁵ are each independently

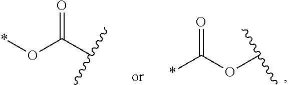 [SMILES: *OC(*)=O ; *OC(*)=O]

In certain embodiments, Y⁴ and Y⁵ are each independently

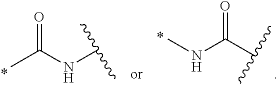

In certain embodiments, Y⁴ and Y⁵ are each independently

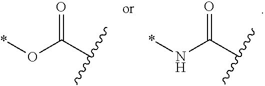 [SMILES: *OC(*)=O ; *NC(*)=O]

In certain embodiments, Y⁴ is

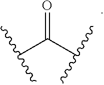 [SMILES: *C(*)=O]

In certain embodiments, Y⁴ is

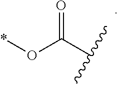 [SMILES: *OC(*)=O]

In certain embodiments, Y⁴ is

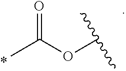 [SMILES: *OC(C)=O]

In certain embodiments, Y⁴ is

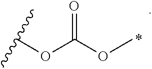

In certain embodiments, Y⁴ is

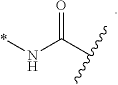 [SMILES: *NC(*)=O]

In certain embodiments, Y⁴ is

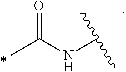 [SMILES: *NC(C)=O]

In certain embodiments, Y⁴ is

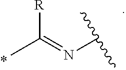

In certain embodiments, Y⁴ is

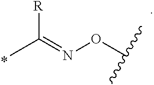

In certain embodiments, Y⁵ is

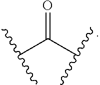 [SMILES: *C(*)=O]

In certain embodiments, Y⁵ is

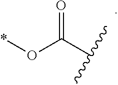 [SMILES: *OC(*)=O]

In certain embodiments, Y⁵ is

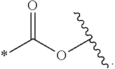 [SMILES: *OC(*)=O]

In certain embodiments, Y⁵ is

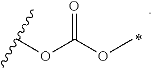

In certain embodiments, Y⁵ is

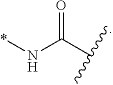 [SMILES: *NC(*)=O]

In certain embodiments, Y⁵ is

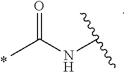 [SMILES: *NC(C)=O]

In certain embodiments, Y⁵ is

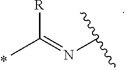

In certain embodiments, Y⁵ is

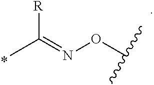

In certain embodiments, Y⁵ and Y⁴ are both

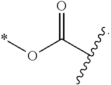 [SMILES: *OC(*)=O]

In certain embodiments, Y⁵ and Y⁴ are both

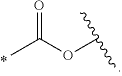 [SMILES: *OC(C)=O]

In certain embodiments, Y⁵ and Y⁴ are both

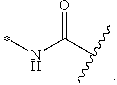

In certain embodiments, Y⁵ and Y⁴ are both

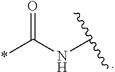 [SMILES: *NC(*)=O]

In certain embodiments, wherein the compound comprises two Y⁵ (in other words, when n=2); each Y⁵ is independently selected from any of the embodiments above, and need not be the same. In certain embodiments, wherein the compound comprises two Y⁵, they are each the same.

### R²

As disclosed in Formula (AX), in certain embodiments, R² is —CH(OR⁶)(OR⁷), —CH(SR⁶)(SR⁷), —CH(R⁶)(R⁷), —R¹⁰, optionally substituted C₅-C₁₈ aliphatic, or optionally substituted C₁-C₁₄ aliphatic-R¹⁰, wherein one or more methylene linkages of R² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R² is —CH(OR⁶)(OR⁷). In certain embodiments, R² is —CH(R⁶)(R⁷). In certain embodiments, R² is —CH(SR⁶)(SR⁷). In certain embodiments, R² is —R¹⁰. In certain embodiments, R² is optionally substituted C₅-C₁₈ aliphatic. In certain embodiments, R² is optionally substituted C₁-C₁₄ aliphatic-R¹⁰.

In certain embodiments, R² is selected from

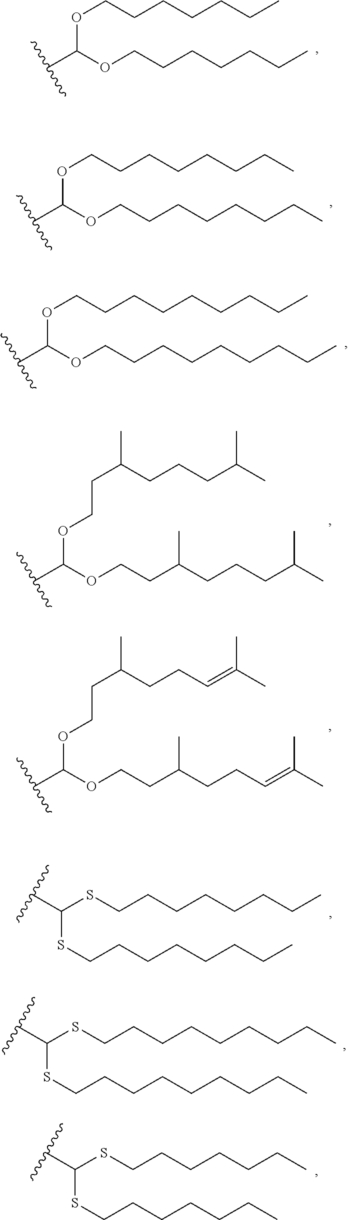

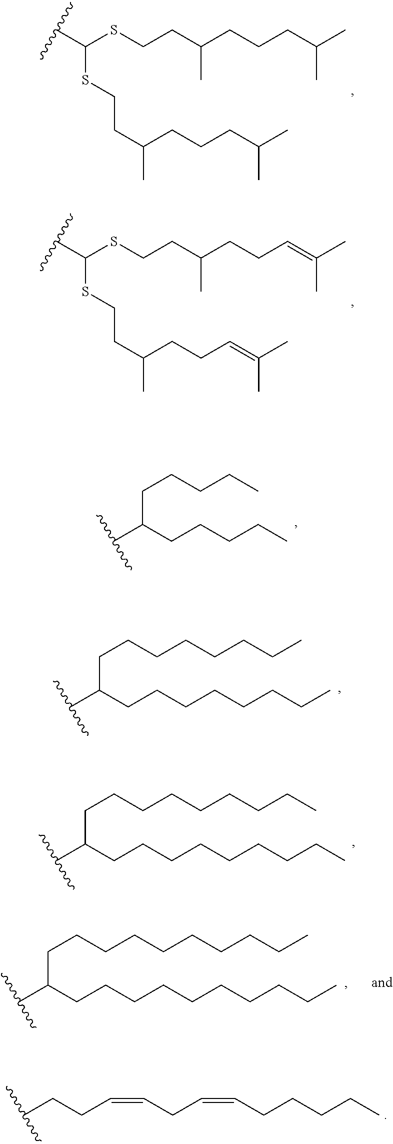

### R³

As disclosed in Formula (AX), in certain embodiments, R³ is —CH(OR⁸)(OR⁹), —CH(SR⁸)(SR⁹), —CH(R⁸)(R⁹), —R¹¹, optionally substituted C₅-C₁₈ aliphatic, or optionally substituted C₁-C₁₄ aliphatic-R¹¹, wherein one or more methylene linkages of R³ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R³ is —CH(OR⁸)(OR⁹). In certain embodiments, R³ is —CH(R⁸)(R⁹). In certain embodiments, R³ is —CH(SR⁸)(SR⁹). In certain embodiments, R³ is —R¹¹. In certain embodiments, R³ is optionally substituted C₅-C₁₈ aliphatic. In certain embodiments, R³ is optionally substituted C₁-C₁₄ aliphatic-R¹¹.

In certain embodiments, R3 is selected from

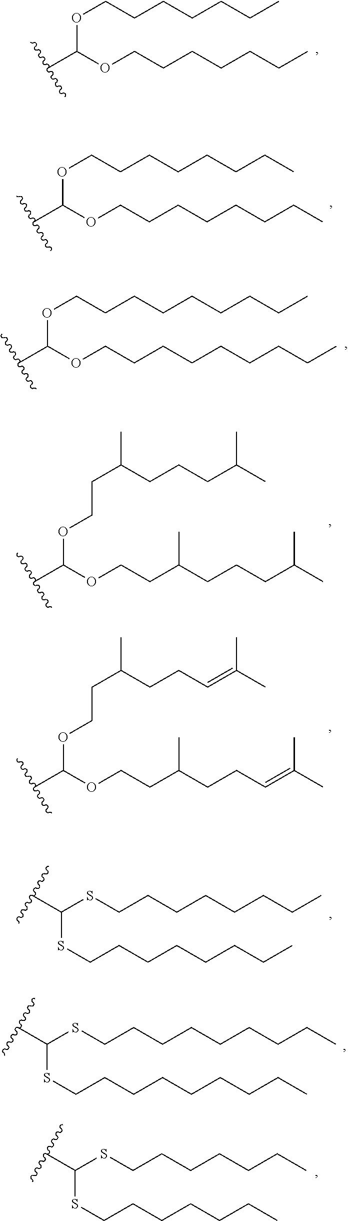

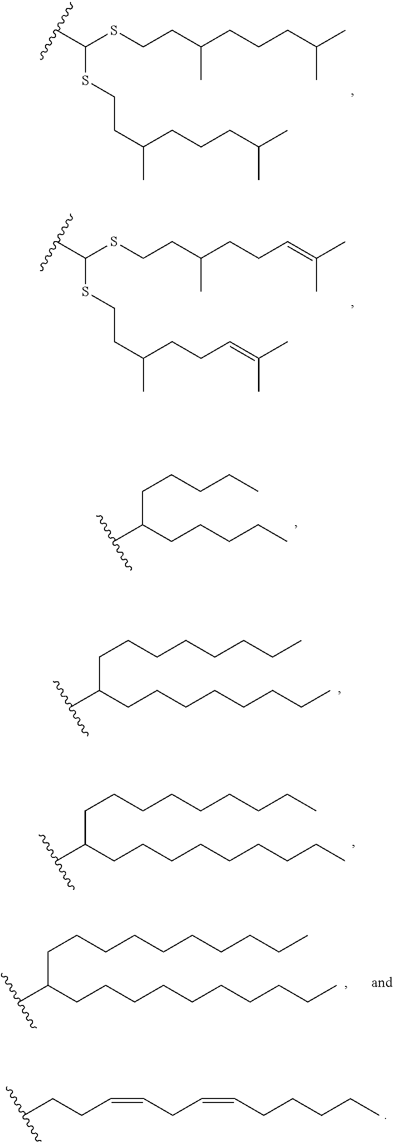

In certain embodiments, R² and R³ are the same. In certain embodiments, R² and R³ are different.

R⁶ and R⁷

As disclosed in Formula (AX), in certain embodiments, R⁶ and R⁷ are each independently optionally substituted —C₁-C₁₄ aliphatic, —R¹⁰, or optionally substituted —C₁-C₁₄ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkyl. In certain embodiments, R⁶ is optionally substituted C₁-C₁₄ branched alkyl. In certain embodiments, R⁶ is optionally substituted C₁-C₁₄ straight chain alkyl. In certain embodiments, R⁶ is optionally substituted C₁-C₁₄ alkenyl. In certain embodiments, R⁶ is optionally substituted C₁-C₁₄ branched alkenyl. In certain embodiments, R⁶ is optionally substituted C₁-C₁₄ straight chain alkenyl. In certain embodiments, R⁶ is optionally substituted C₄-C₁₀ alkyl. In certain embodiments, R⁶ is optionally substituted straight chain C₄-C₁₀ alkyl. In certain embodiments, R⁶ is unsubstituted C₆-C₁₀ alkyl. In certain embodiments, R⁶ is unsubstituted straight chain C₄-C₁₀ alkyl. In certain embodiments, R⁶ is optionally substituted C₆-C₁₀ alkyl. In certain embodiments, R⁶ is optionally substituted straight chain C₆-C₁₀ alkyl. In certain embodiments, R⁶ is unsubstituted C₄-C₁₀ alkyl. In certain embodiments, R⁶ is unsubstituted straight chain C₆-C₁₀ alkyl. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, R⁷ is optionally substituted C₁-C₁₄ aliphatic. In certain embodiments, R⁷ is optionally substituted C₁-C₁₄ alkyl. In certain embodiments, R⁷ is optionally substituted C₁-C₁₄ branched alkyl. In certain embodiments, R⁷ is optionally substituted C₁-C₁₄ straight chain alkyl. In certain embodiments, R⁷ is optionally substituted C₁-C₁₄ alkenyl. In certain embodiments, R⁷ is optionally substituted C₁-C₁₄ branched alkenyl. In certain embodiments, R⁷ is optionally substituted C₁-C₁₄ straight chain alkenyl. In certain embodiments, R⁷ is optionally substituted C₄-C₁₀ alkyl. In certain embodiments, R⁷ is optionally substituted straight chain C₄-C₁₀ alkyl. In certain embodiments, R⁷ is unsubstituted C₆-C₁₀ alkyl. In certain embodiments, R⁷ is unsubstituted straight chain C₄-C₁₀ alkyl. In certain embodiments, R⁷ is optionally substituted C₆-C₁₀ alkyl. In certain embodiments, R⁷ is optionally substituted straight chain C₆-C₁₀ alkyl. In certain embodiments, R⁷ is unsubstituted C₄-C₁₀ alkyl. In certain embodiments, R⁷ is unsubstituted straight chain C₆-C₁₀ alkyl. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁷ is optionally substituted —(CH₂)₉CH₃.

In certain embodiments, each R⁶ and R⁷ are each independently selected from

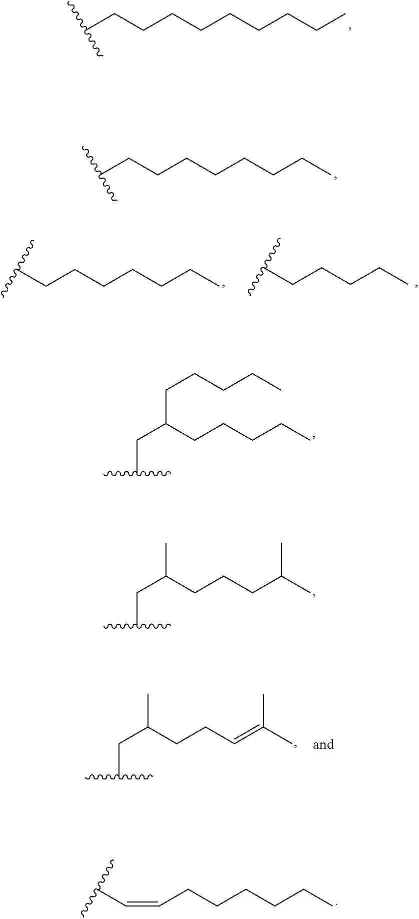

R⁸ and R⁹

As disclosed in Formula (AX), in certain embodiments, R⁸ and R⁹ are each independently optionally substituted —C₁-C₁₄ aliphatic, —R¹¹, or optionally substituted —C₁-C₁₄ aliphatic-R¹¹; wherein one or more methylene linkages of R⁸ and R⁹ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₄-C₁₀ alkyl. In certain embodiments, R⁸ is optionally substituted straight chain C₄-C₁₀ alkyl. In certain embodiments, R⁸ is unsubstituted C₆-C₁₀ alkyl. In certain embodiments, R⁸ is unsubstituted straight chain C₄-C₁₀ alkyl. In certain embodiments, R⁸ is optionally substituted C₆-C₁₀ alkyl. In certain embodiments, R⁸ is optionally substituted straight chain C₆-C₁₀ alkyl. In certain embodiments, R⁸ is unsubstituted C₄-C₁₀ alkyl. In certain embodiments, R⁸ is unsubstituted straight chain C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₄-C₁₀ alkyl. In certain embodiments, R⁹ is optionally substituted straight chain C₄-C₁₀ alkyl. In certain embodiments, R⁹ is unsubstituted C₆-C₁₀ alkyl. In certain embodiments, R⁹ is unsubstituted straight chain C₄-C₁₀ alkyl. In certain embodiments, R⁹ is optionally substituted C₆-C₁₀ alkyl. In certain embodiments, R⁹ is optionally substituted straight chain C₆-C₁₀ alkyl. In certain embodiments, R⁹ is unsubstituted C₄-C₁₀ alkyl. In certain embodiments, R⁹ is unsubstituted straight chain C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, each R⁸ and R⁹ are each independently selected from

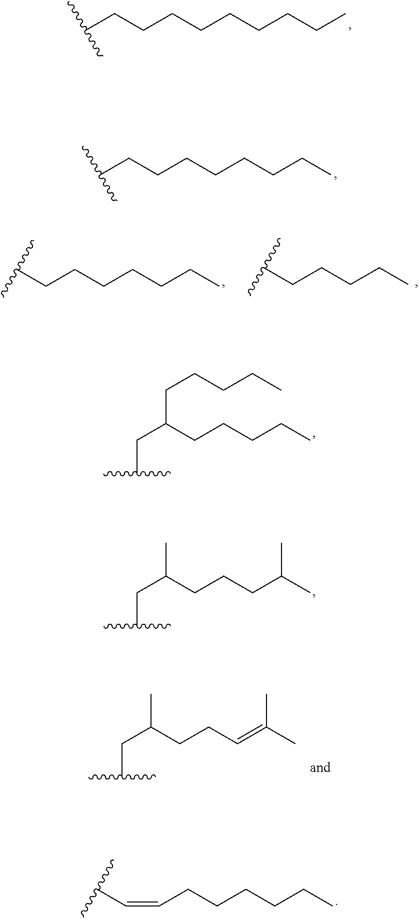

R¹⁰ and R¹¹

As disclosed in Formula (AX), in certain embodiments, each R¹⁰ and R¹ are independently an optionally substituted cyclic, bicyclic, bridged bicyclic, multicyclic or bridged multicyclic C₄-C₁₄ cycloalkyl or optionally substituted cylic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R¹⁰ and R¹¹ are independently an optionally substituted monocyclic C₄-C₁₄ cycloalkyl. In certain embodiments, each R¹⁰ and R¹¹ are independently an optionally substituted monocyclic C₆-C₁₄ cycloalkyl. In certain embodiments, each R¹⁰ and R¹¹ are independently an optionally substituted monocyclic C₆-C₈ cycloalkyl. In certain embodiments, each R¹⁰ and R¹¹ are independently an optionally substituted bicyclic C₄-C₁₄ cycloalkyl. In certain embodiments, each R¹⁰ and R¹¹ are independently an optionally substituted bicyclic C₆-C₁₄ cycloalkyl. In certain embodiments, each R¹⁰ and R¹¹ are independently an optionally substituted bicyclic C₈-C₁₄ cycloalkyl. In certain embodiments, each R¹⁰ and R¹¹ are independently an optionally substituted bicyclic C₆-C₁₀ cycloalkyl. In certain embodiments, each R¹⁰ and R¹¹ are independently an optionally substituted bridged bicyclic C₄-C₁₄ cycloalkyl. In certain embodiments, each R¹⁰ and R¹¹ are independently an optionally substituted bridged bicyclic C₆-C₁₄ cycloalkyl. In certain embodiments, each R¹⁰ and R¹¹ are independently an optionally substituted bridged bicyclic C₈-C₁₄ cycloalkyl. In certain embodiments, each R¹⁰ and R¹ are independently an optionally substituted bridged bicyclic C₆-C₁₀ cycloalkyl. In certain embodiments, each R¹⁰ and R¹¹ are independently an optionally substituted multicyclic C₄-C₁₄ cycloalkyl. In certain embodiments, each R¹⁰ and R¹ are independently an optionally substituted multicyclic C₆-C₁₄ cycloalkyl. In certain embodiments, each R¹⁰ and R¹¹ are independently an optionally substituted multicyclic C₈-C₁₄ cycloalkyl. In certain embodiments, each R¹⁰ and R¹¹ are independently an optionally substituted bridged multicyclic C₄-C₁₄ cycloalkyl. In certain embodiments, each R¹⁰ and R¹¹ are independently an optionally substituted bridged multicyclic C₆-C₁₄ cycloalkyl. In certain embodiments, each R¹⁰ and R¹¹ are independently an optionally substituted bridged multicyclic C₈-C₁₄ cycloalkyl. In certain embodiments, two R¹⁰ or two R¹¹ taken together form an optionally substituted bridged bicyclic or multicyclic C₄-C₁₄ cycloalkyl. In certain embodiments, two R¹⁰ or two R¹¹ taken together form an optionally substituted bridged bicyclic or multicyclic C₆-C₁₄ cycloalkyl. In certain embodiments, two R¹⁰ or two R¹¹ taken together form an optionally substituted bridged bicyclic or multicyclic C₈-C₁₄ cycloalkyl.

In certain embodiments, each R¹⁰ and R¹¹ are independently an optionally substituted monocyclic 4-14 membered heterocyclyl. In certain embodiments, each R¹⁰ and R¹¹ are independently an optionally substituted monocyclic 6-14 membered heterocyclyl. In certain embodiments, each R¹⁰ and R¹¹ are independently an optionally substituted monocyclic C₆-C₈ heterocyclyl. In certain embodiments, each R¹⁰ and R¹¹ are independently an optionally substituted bicyclic 4-14 membered heterocyclyl. In certain embodiments, each R¹⁰ and R¹¹ are independently an optionally substituted bicyclic 6-14 membered heterocyclyl. In certain embodiments, each R¹⁰ and R¹¹ are independently an optionally substituted bicyclic C₈-C₁₄ heterocyclyl. In certain embodiments, each R¹⁰ and R¹¹ are independently an optionally substituted bicyclic 6-12 membered heterocyclyl. In certain embodiments, each R¹⁰ and R¹¹ are independently an optionally substituted bridged bicyclic 4-14 membered heterocyclyl. In certain embodiments, each R¹⁰ and R¹¹ are independently an optionally substituted bridged bicyclic 6-14 membered heterocyclyl. In certain embodiments, each R¹⁰ and R¹¹ are independently an optionally substituted bridged bicyclic C₈-C₁₄ heterocyclyl. In certain embodiments, each R¹⁰ and R¹¹ are independently an optionally substituted bridged bicyclic 6-12 membered heterocyclyl. In certain embodiments, each R¹⁰ and R¹¹ are independently an optionally substituted multicyclic 4-14 membered heterocyclyl. In certain embodiments, each R¹⁰ and R¹¹ are independently an optionally substituted multicyclic 6-14 membered heterocyclyl. In certain embodiments, each R¹⁰ and R¹¹ are independently an optionally substituted multicyclic C₈-C₁₄ heterocyclyl. In certain embodiments, each R¹⁰ and R¹¹ are independently an optionally substituted bridged multicyclic 4-14 membered heterocyclyl. In certain embodiments, each R¹⁰ and R¹¹ are independently an optionally substituted bridged multicyclic 6-14 membered heterocyclyl. In certain embodiments, each R¹⁰ and R¹¹ are independently an optionally substituted bridged multicyclic C₈-C₁₄ heterocyclyl. In certain embodiments, two R¹⁰ or two R¹¹ taken together form an optionally substituted bridged bicyclic or multicyclic 4-14 membered heterocyclyl. In certain embodiments, two R¹⁰ or two R¹¹ taken together form an optionally substituted bridged bicyclic or multicyclic 6-14 membered heterocyclyl. In certain embodiments, two R¹⁰ or two R¹¹ taken together form an optionally substituted bridged bicyclic or multicyclic 8-14 membered heterocyclyl.

In certain embodiments, each R¹⁰ and R¹¹ is independently an optionally substituted monovalent cyclic group selected from

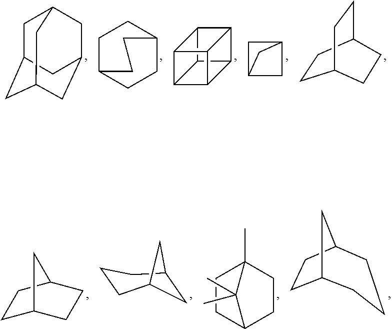

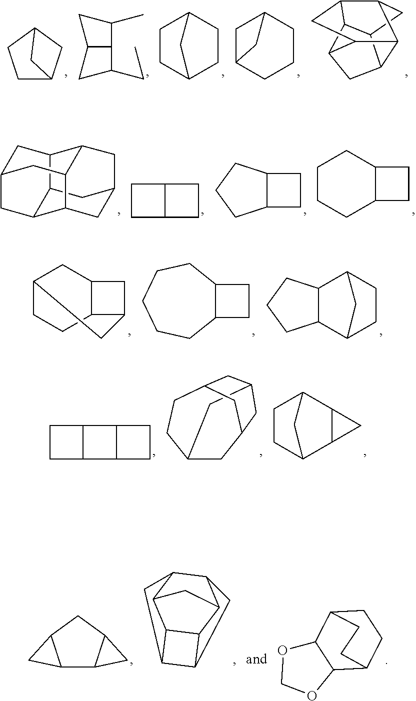

In certain embodiments, each R¹⁰ and R¹¹ is independently a structure selected from

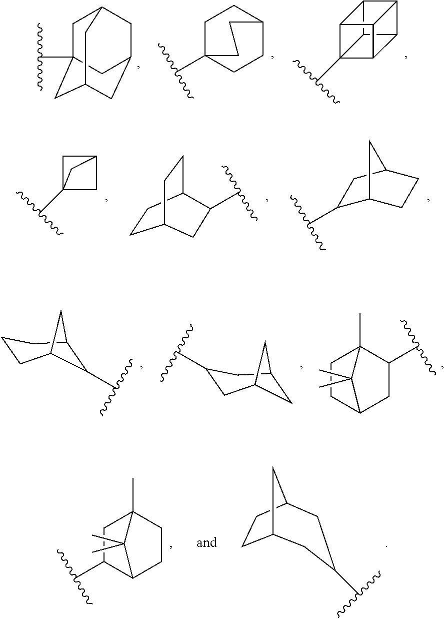

In certain embodiments, each R¹⁰ and R¹¹ is independently a structure selected from

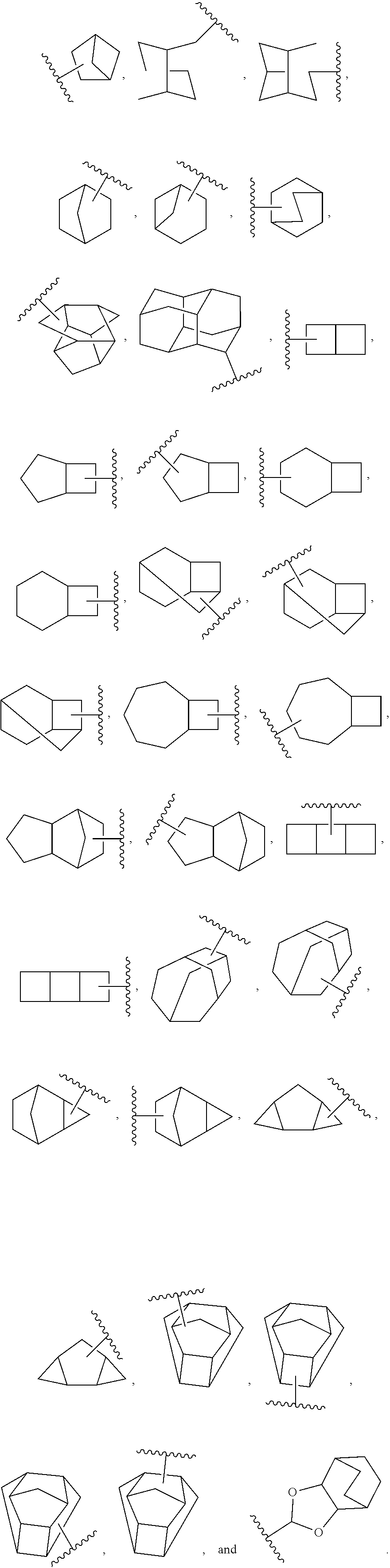

In certain embodiments, each R¹⁰ and R¹¹ is independently a structure selected from

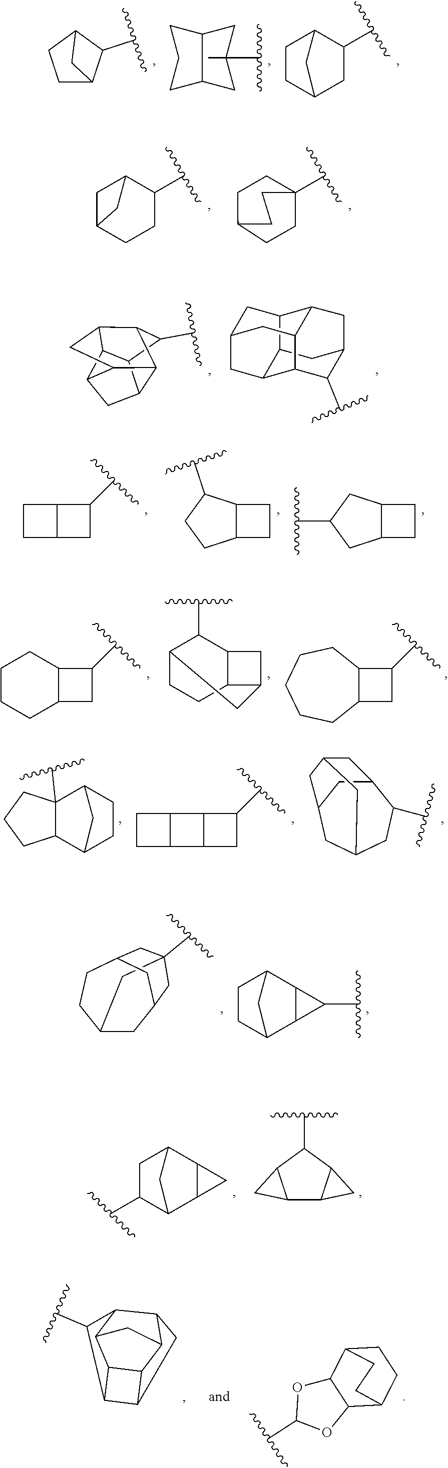

In certain embodiments, each R¹⁰ and R¹¹ is independently a structure selected from and

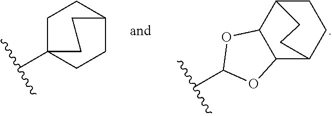 [SMILES: *C12CCC(CC1)CC2 ; *C1OC2C3CCC(CC3)C2O1]

In some embodiments, ionizable lipids of the present disclosure are selected from any lipid in Table (I-L) below or a pharmaceutically acceptable salt, solvate, stereoisomer, or enantiomer thereof:

TABLE (I-L)
Non-Limiting Examples of Ionizable lipids of the present disclosure

| Comp # | Structure |
|---|---|
| AX-1 | 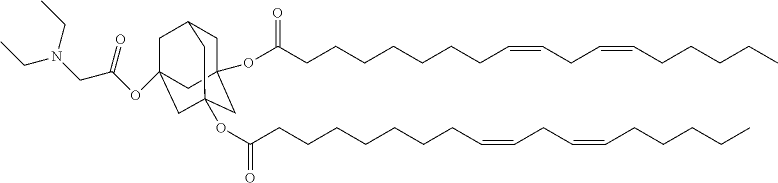 [SMILES: CCCCC/C=C\C/C=C\CCCCCCCC(=O)OC12CC3CC(OC(=O)CCCCCCC/C=C\C/C=C\CCCCC)(C1)CC(OC(=O)CN(CC)CC)(C3)C2] |
| AX-2 | 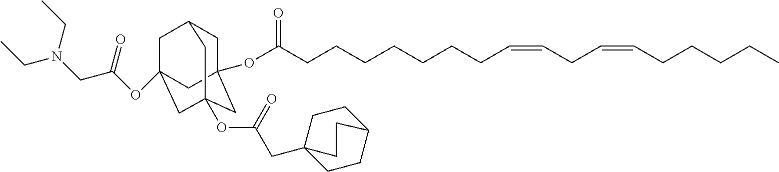 [SMILES: CCCCC/C=C\C/C=C\CCCCCCCC(=O)OC12CC3CC(OC(=O)CN(CC)CC)(C1)CC(OC(=O)CC14CCC(CC1)CC4)(C3)C2] |
| AX-3 | 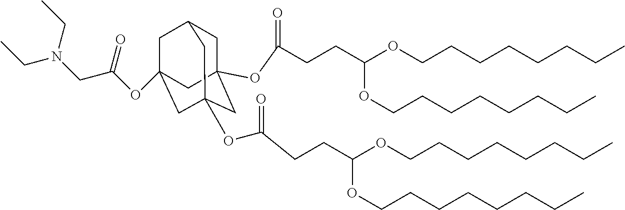 [SMILES: CCCCCCCCOC(CCC(=O)OC12CC3CC(OC(=O)CCC(OCCCCCCCC)OCCCCCCCC)(C1)CC(OC(=O)CN(CC)CC)(C3)C2)OCCCCCCCC] |
| AX-4 | 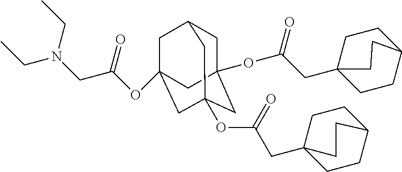 [SMILES: CCN(CC)CC(=O)OC12CC3CC(OC(=O)CC45CCC(CC4)CC5)(C1)CC(OC(=O)CC14CCC(CC1)CC4)(C3)C2] |
| AX-5 | 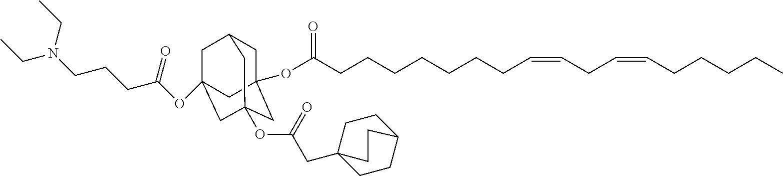 [SMILES: CCCCC/C=C\C/C=C\CCCCCCCC(=O)OC12CC3CC(OC(=O)CCCN(CC)CC)(C1)CC(OC(=O)CC14CCC(CC1)CC4)(C3)C2] |
| AX-6 | 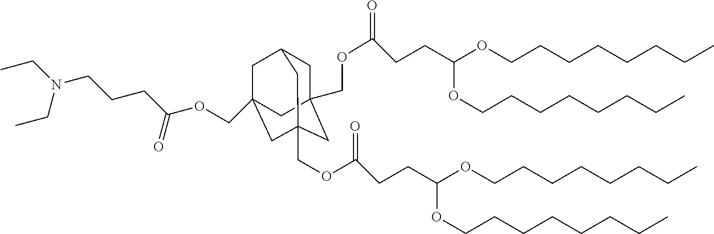 [SMILES: CCCCCCCCOC(CCC(=O)OCC12CC3CC(COC(=O)CCCN(CC)CC)(C1)CC(COC(=O)CCC(OCCCCCCCC)OCCCCCCCC)(C3)C2)OCCCCCCCC] |
| AX-7 | 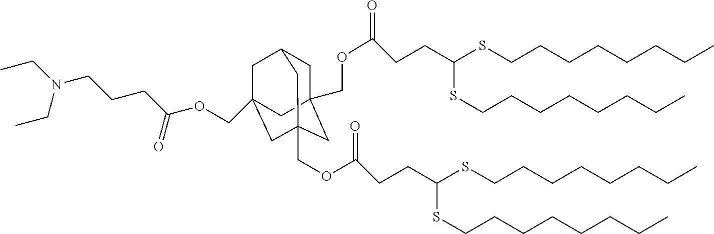 [SMILES: CCCCCCCCSC(CCC(=O)OCC12CC3CC(COC(=O)CCCN(CC)CC)(C1)CC(COC(=O)CCC(SCCCCCCCC)SCCCCCCCC)(C3)C2)SCCCCCCCC] |
| AX-8 | 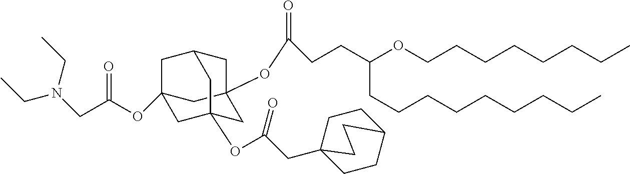 [SMILES: CCCCCCCCCC(CCC(=O)OC12CC3CC(OC(=O)CN(CC)CC)(C1)CC(OC(=O)CC14CCC(CC1)CC4)(C3)C2)OCCCCCCCC] |
| AX-9 | 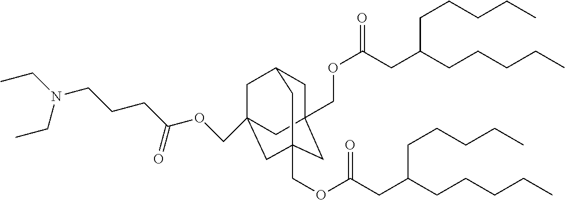 [SMILES: CCCCCC(CCCCC)CC(=O)OCC12CC3CC(COC(=O)CCCN(CC)CC)(C1)CC(COC(=O)CC(CCCCC)CCCCC)(C3)C2] |
| AX-10 | 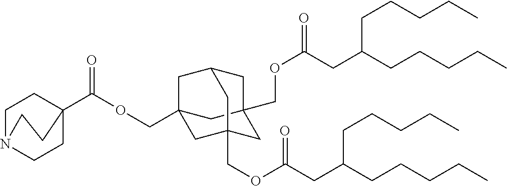 [SMILES: CCCCCC(CCCCC)CC(=O)OCC12CC3CC(COC(=O)CC(CCCCC)CCCCC)(C1)CC(COC(=O)C14CCN(CC1)CC4)(C3)C2] |
| AX-11 | 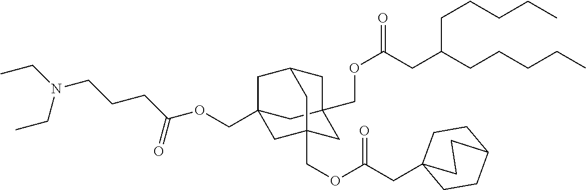 [SMILES: CCCCCC(CCCCC)CC(=O)OCC12CC3CC(COC(=O)CCCN(CC)CC)(C1)CC(COC(=O)CC14CCC(CC1)CC4)(C3)C2] |
| AX-12 | 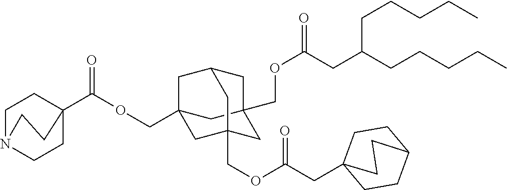 [SMILES: CCCCCC(CCCCC)CC(=O)OCC12CC3CC(COC(=O)CC45CCC(CC4)CC5)(C1)CC(COC(=O)C14CCN(CC1)CC4)(C3)C2] |
| AX-13 | 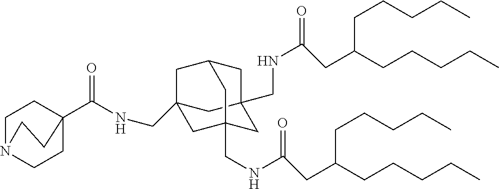 [SMILES: CCCCCC(CCCCC)CC(=O)NCC12CC3CC(CNC(=O)CC(CCCCC)CCCCC)(C1)CC(CNC(=O)C14CCN(CC1)CC4)(C3)C2] |
| AX-14 | 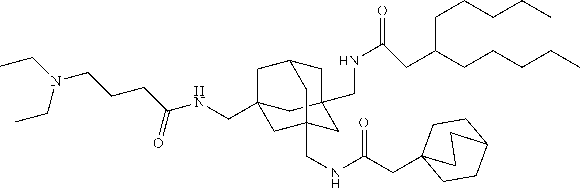 [SMILES: CCCCCC(CCCCC)CC(=O)NCC12CC3CC(CNC(=O)CCCN(CC)CC)(C1)CC(CNC(=O)CC14CCC(CC1)CC4)(C3)C2] |
| AX-15 | 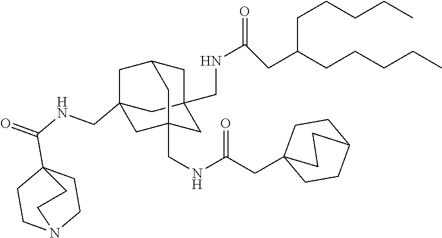 [SMILES: CCCCCC(CCCCC)CC(=O)NCC12CC3CC(CNC(=O)CC45CCC(CC4)CC5)(C1)CC(CNC(=O)C14CCN(CC1)CC4)(C3)C2] |
| AX-16 | 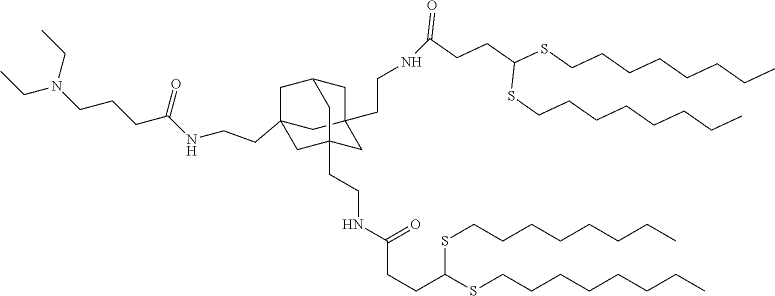 [SMILES: CCCCCCCCSC(CCC(=O)NCCC12CC3CC(CCNC(=O)CCCN(CC)CC)(C1)CC(CCNC(=O)CCC(SCCCCCCCC)SCCCCCCCC)(C3)C2)SCCCCCCCC] |
| AX-17 | 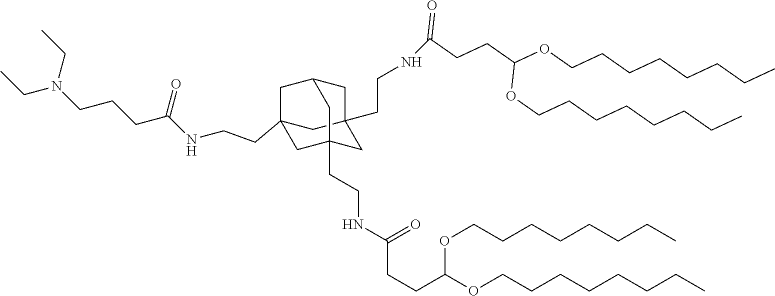 [SMILES: CCCCCCCCOC(CCC(=O)NCCC12CC3CC(CCNC(=O)CCCN(CC)CC)(C1)CC(CCNC(=O)CCC(OCCCCCCCC)OCCCCCCCC)(C3)C2)OCCCCCCCC] |
| AX-18 | 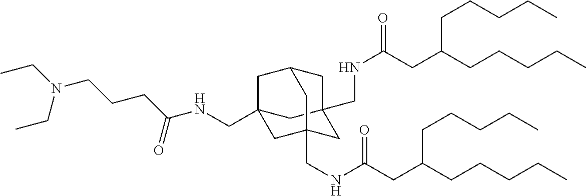 [SMILES: CCCCCC(CCCCC)CC(=O)NCC12CC3CC(CNC(=O)CCCN(CC)CC)(C1)CC(CNC(=O)CC(CCCCC)CCCCC)(C3)C2] |
| AX-19 | 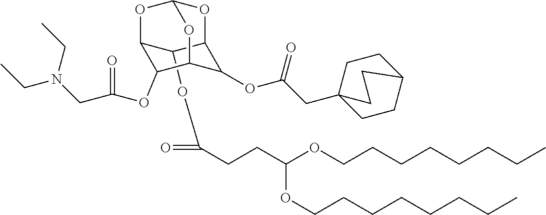 [SMILES: CCCCCCCCOC(CCC(=O)OC1C2OC3OC1C(OC(=O)CC14CCC(CC1)CC4)C(O3)C2OC(=O)CN(CC)CC)OCCCCCCCC] |
| AX-20 | 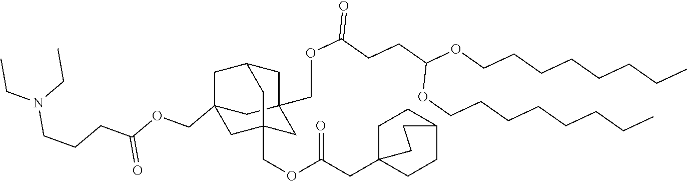 [SMILES: CCCCCCCCOC(CCC(=O)OCC12CC3CC(COC(=O)CCCN(CC)CC)(C1)CC(COC(=O)CC14CCC(CC1)CC4)(C3)C2)OCCCCCCCC] |
| AX-21 | 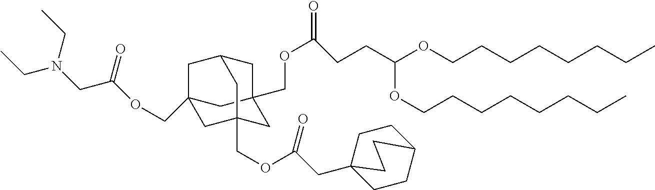 [SMILES: CCCCCCCCOC(CCC(=O)OCC12CC3CC(COC(=O)CN(CC)CC)(C1)CC(COC(=O)CC14CCC(CC1)CC4)(C3)C2)OCCCCCCCC] |
| AX-22 | 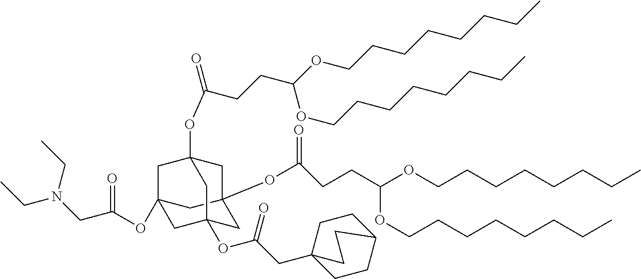 [SMILES: CCCCCCCCOC(CCC(=O)OC12CC3(OC(=O)CCC(OCCCCCCCC)OCCCCCCCC)CC(OC(=O)CN(CC)CC)(C1)CC(OC(=O)CC14CCC(CC1)CC4)(C2)C3)OCCCCCCCC] |
| AX-23 | 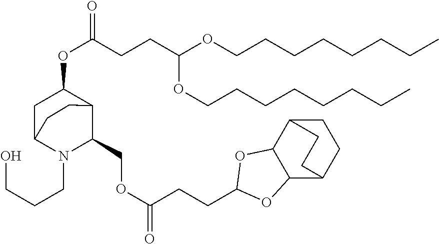 [SMILES: CCCCCCCCOC(CCC(=O)O[C@@H]1CC2CCC1[C@@H](COC(=O)CCC1OC3C4CCC(CC4)C3O1)N2CCCO)OCCCCCCCC] |
| AX-24 | 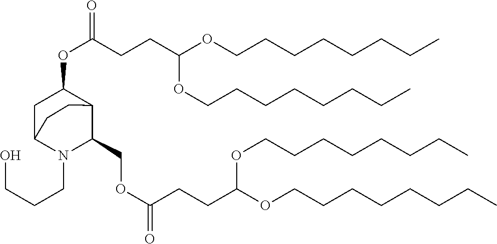 [SMILES: CCCCCCCCOC(CCC(=O)OC[C@@H]1C2CCC(C[C@H]2OC(=O)CCC(OCCCCCCCC)OCCCCCCCC)N1CCCO)OCCCCCCCC] |
| AX-25 | 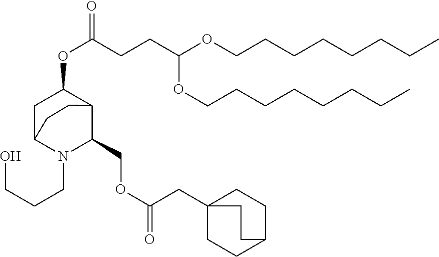 [SMILES: CCCCCCCCOC(CCC(=O)O[C@@H]1CC2CCC1[C@@H](COC(=O)CC13CCC(CC1)CC3)N2CCCO)OCCCCCCCC] |
| AX-26 | 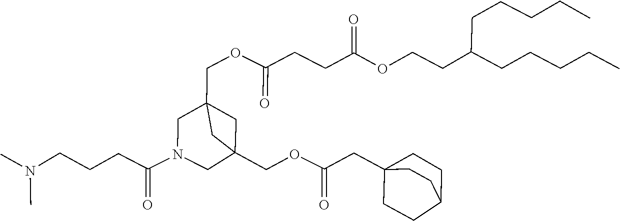 [SMILES: CCCCCC(CCCCC)CCOC(=O)CCC(=O)OCC12CN(C(=O)CCCN(C)C)CC(COC(=O)CC34CCC(CC3)CC4)(C1)C2] |
| AX-27 | 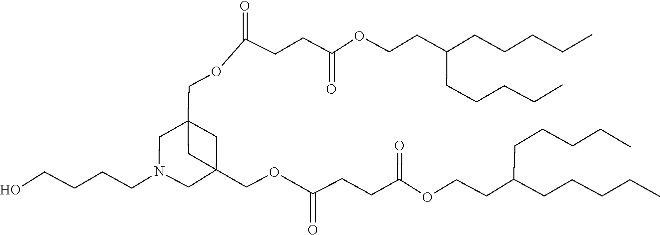 [SMILES: CCCCCC(CCCCC)CCOC(=O)CCC(=O)OCC12CN(CCCCO)CC(COC(=O)CCC(=O)OCCC(CCCCC)CCCCC)(C1)C2] |
| AX-28 | 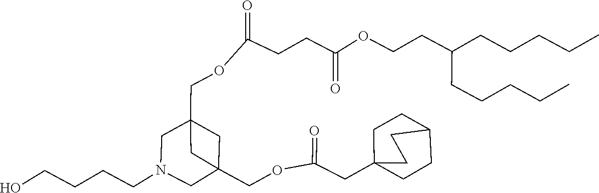 [SMILES: CCCCCC(CCCCC)CCOC(=O)CCC(=O)OCC12CN(CCCCO)CC(COC(=O)CC34CCC(CC3)CC4)(C1)C2] |
| AX-29 | 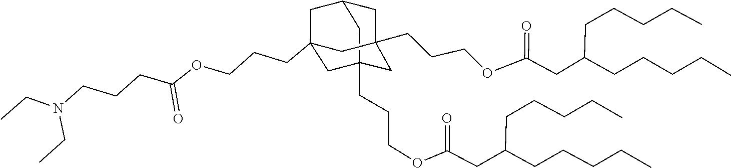 [SMILES: CCCCCC(CCCCC)CC(=O)OCCCC12CC3CC(CCCOC(=O)CCCN(CC)CC)(C1)CC(CCCOC(=O)CC(CCCCC)CCCCC)(C3)C2] |
| AX-30 | 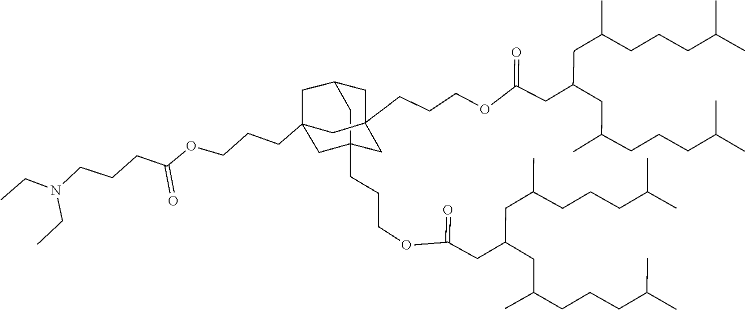 [SMILES: CCN(CC)CCCC(=O)OCCCC12CC3CC(CCCOC(=O)CC(CC(C)CCCC(C)C)CC(C)CCCC(C)C)(C1)CC(CCCOC(=O)CC(CC(C)CCCC(C)C)CC(C)CCCC(C)C)(C3)C2] |
| AX-31 | 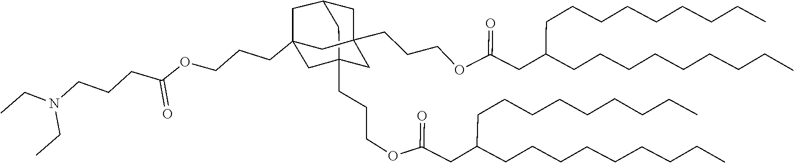 [SMILES: CCCCCCCCCC(CCCCCCCCC)CC(=O)OCCCC12CC3CC(CCCOC(=O)CCCN(CC)CC)(C1)CC(CCCOC(=O)CC(CCCCCCCCC)CCCCCCCCC)(C3)C2] |
| AX-32 | 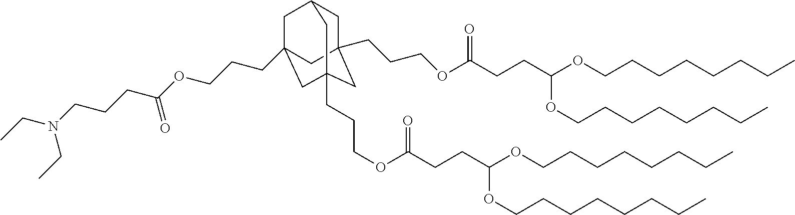 [SMILES: CCCCCCCCOC(CCC(=O)OCCCC12CC3CC(CCCOC(=O)CCCN(CC)CC)(C1)CC(CCCOC(=O)CCC(OCCCCCCCC)OCCCCCCCC)(C3)C2)OCCCCCCCC] |
| AX-33 | 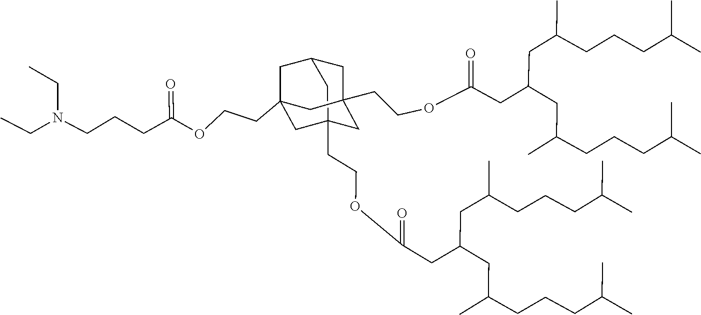 [SMILES: CCN(CC)CCCC(=O)OCCC12CC3CC(CCOC(=O)CC(CC(C)CCCC(C)C)CC(C)CCCC(C)C)(C1)CC(CCOC(=O)CC(CC(C)CCCC(C)C)CC(C)CCCC(C)C)(C3)C2] |
| AX-34 | 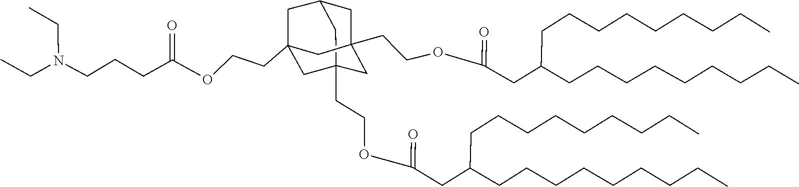 [SMILES: CCCCCCCCCC(CCCCCCCCC)CC(=O)OCCC12CC3CC(CCOC(=O)CCCN(CC)CC)(C1)CC(CCOC(=O)CC(CCCCCCCCC)CCCCCCCCC)(C3)C2] |
| AX-35 | 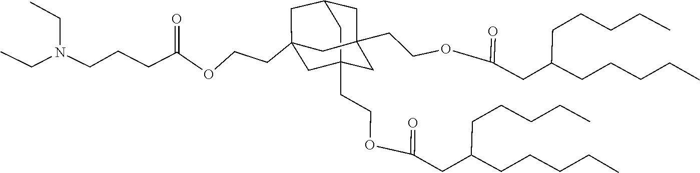 [SMILES: CCCCCC(CCCCC)CC(=O)OCCC12CC3CC(CCOC(=O)CCCN(CC)CC)(C1)CC(CCOC(=O)CC(CCCCC)CCCCC)(C3)C2] |
| AX-36 | 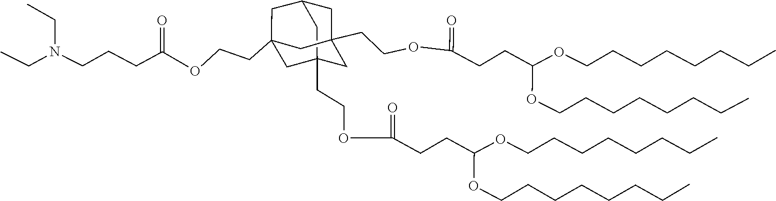 [SMILES: CCCCCCCCOC(CCC(=O)OCCC12CC3CC(CCOC(=O)CCCN(CC)CC)(C1)CC(CCOC(=O)CCC(OCCCCCCCC)OCCCCCCCC)(C3)C2)OCCCCCCCC] |
| AX-37 | 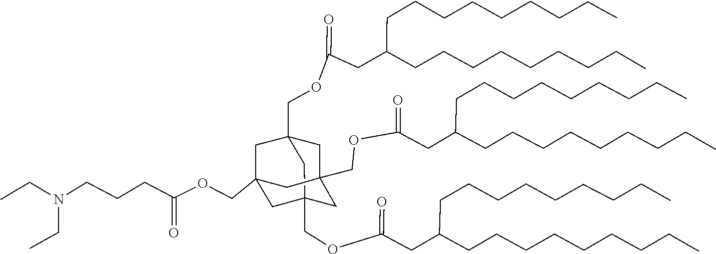 [SMILES: CCCCCCCCCC(CCCCCCCCC)CC(=O)OCC12CC3(COC(=O)CCCN(CC)CC)CC(COC(=O)CC(CCCCCCCCC)CCCCCCCCC)(C1)CC(COC(=O)CC(CCCCCCCCC)CCCCCCCCC)(C3)C2] |
| AX-38 | 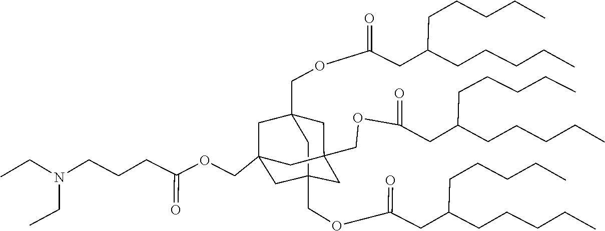 [SMILES: CCCCCC(CCCCC)CC(=O)OCC12CC3(COC(=O)CCCN(CC)CC)CC(COC(=O)CC(CCCCC)CCCCC)(C1)CC(COC(=O)CC(CCCCC)CCCCC)(C3)C2] |
| AX-39 | 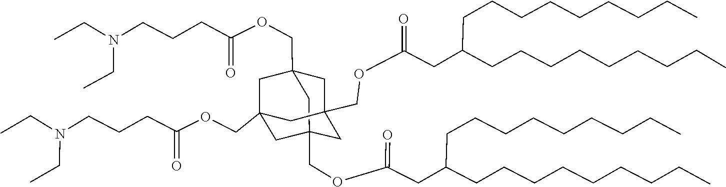 [SMILES: CCCCCCCCCC(CCCCCCCCC)CC(=O)OCC12CC3(COC(=O)CCCN(CC)CC)CC(COC(=O)CCCN(CC)CC)(C1)CC(COC(=O)CC(CCCCCCCCC)CCCCCCCCC)(C3)C2] |
| AX-40 | 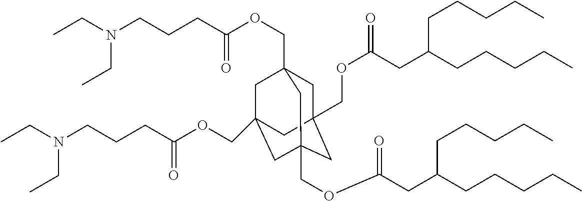 [SMILES: CCCCCC(CCCCC)CC(=O)OCC12CC3(COC(=O)CCCN(CC)CC)CC(COC(=O)CCCN(CC)CC)(C1)CC(COC(=O)CC(CCCCC)CCCCC)(C3)C2] |
| AX-41 | 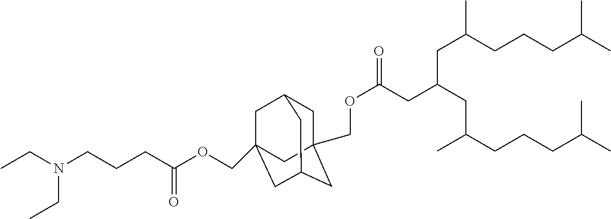 [SMILES: CCN(CC)CCCC(=O)OCC12CC3CC(C1)CC(COC(=O)CC(CC(C)CCCC(C)C)CC(C)CCCC(C)C)(C3)C2] |
| AX-42 | 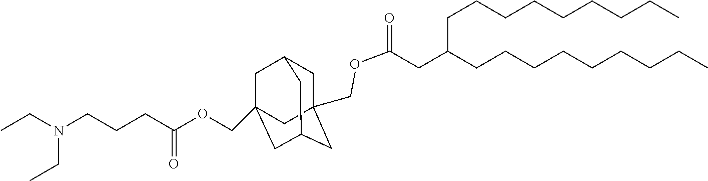 [SMILES: CCCCCCCCCC(CCCCCCCCC)CC(=O)OCC12CC3CC(CC(COC(=O)CCCN(CC)CC)(C3)C1)C2] |
| AX-43 | 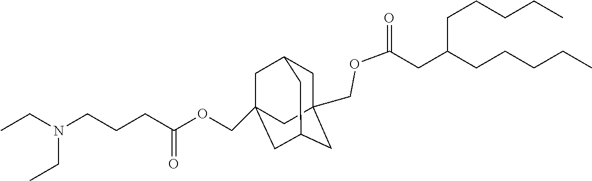 [SMILES: CCCCCC(CCCCC)CC(=O)OCC12CC3CC(CC(COC(=O)CCCN(CC)CC)(C3)C1)C2] |
| AX-44 | 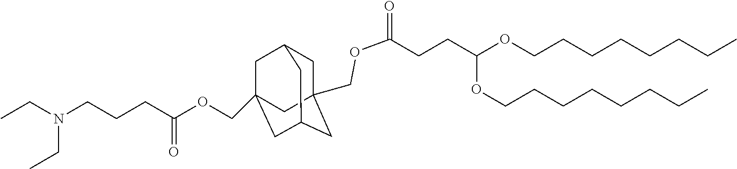 [SMILES: CCCCCCCCOC(CCC(=O)OCC12CC3CC(CC(COC(=O)CCCN(CC)CC)(C3)C1)C2)OCCCCCCCC] |
| AX-45 | 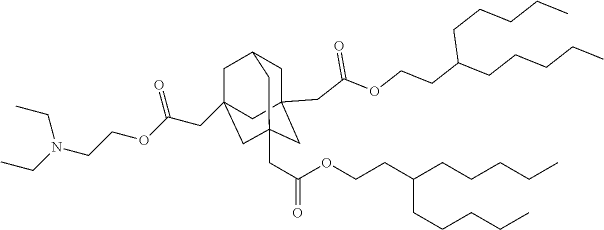 [SMILES: CCCCCC(CCCCC)CCOC(=O)CC12CC3CC(CC(=O)OCCC(CCCCC)CCCCC)(C1)CC(CC(=O)OCCN(CC)CC)(C3)C2] |
| AX-46 | 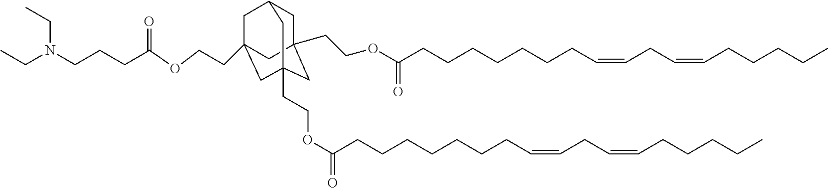 [SMILES: CCCCC/C=C\C/C=C\CCCCCCCC(=O)OCCC12CC3CC(CCOC(=O)CCCCCCC/C=C\C/C=C\CCCCC)(C1)CC(CCOC(=O)CCCN(CC)CC)(C3)C2] |
| AX-47 | 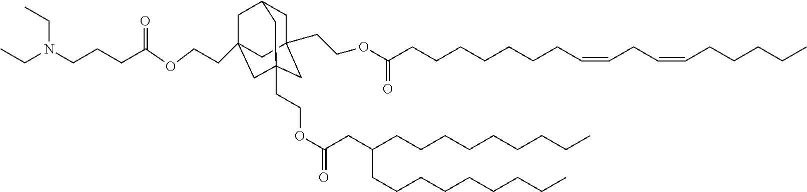 [SMILES: CCCCC/C=C\C/C=C\CCCCCCCC(=O)OCCC12CC3CC(CCOC(=O)CCCN(CC)CC)(C1)CC(CCOC(=O)CC(CCCCCCCCC)CCCCCCCCC)(C3)C2] |
| AX-48 | 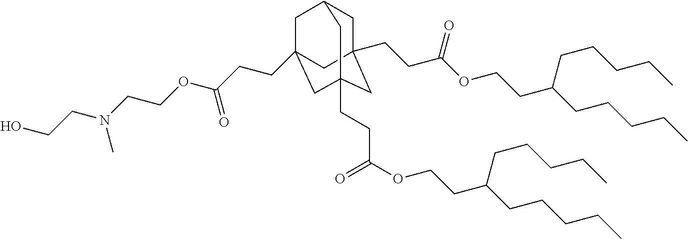 [SMILES: CCCCCC(CCCCC)CCOC(=O)CCC12CC3CC(CCC(=O)OCCC(CCCCC)CCCCC)(C1)CC(CCC(=O)OCCN(C)CCO)(C3)C2] |
| AX-49 | 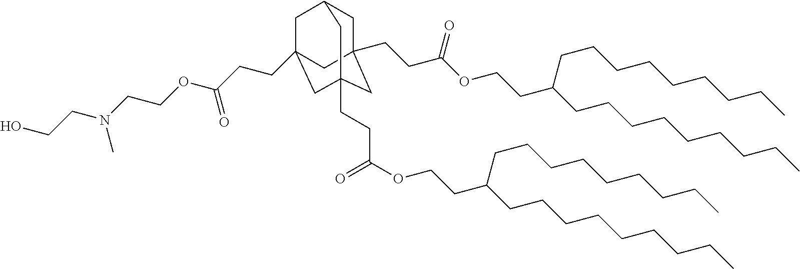 [SMILES: CCCCCCCCCC(CCCCCCCCC)CCOC(=O)CCC12CC3CC(CCC(=O)OCCC(CCCCCCCCC)CCCCCCCCC)(C1)CC(CCC(=O)OCCN(C)CCO)(C3)C2] |
| AX-50 | 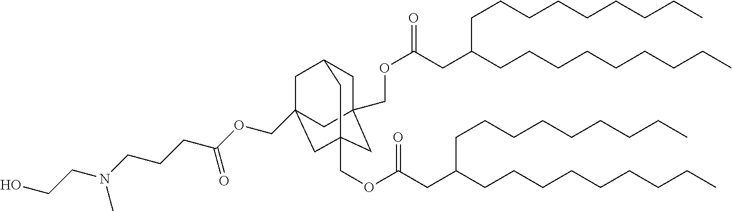 [SMILES: CCCCCCCCCC(CCCCCCCCC)CC(=O)OCC12CC3CC(COC(=O)CCCN(C)CCO)(C1)CC(COC(=O)CC(CCCCCCCCC)CCCCCCCCC)(C3)C2] |
| AX-51 | 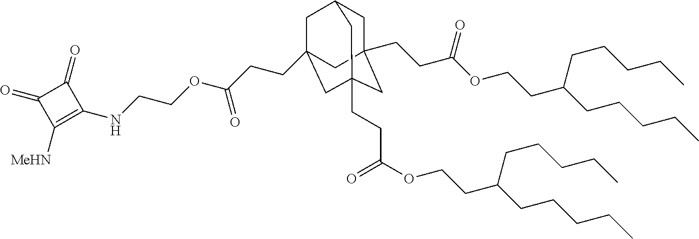 [SMILES: CCCCCC(CCCCC)CCOC(=O)CCC12CC3CC(CCC(=O)OCCNc4c(NC)c(=O)c4=O)(C1)CC(CCC(=O)OCCC(CCCCC)CCCCC)(C3)C2] |
| AX-52 | 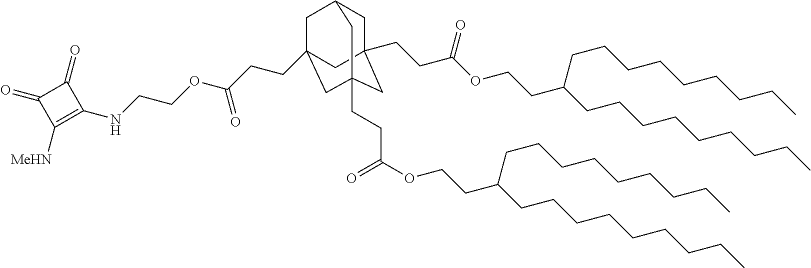 [SMILES: CCCCCCCCCC(CCCCCCCCC)CCOC(=O)CCC12CC3CC(CCC(=O)OCCNc4c(NC)c(=O)c4=O)(C1)CC(CCC(=O)OCCC(CCCCCCCCC)CCCCCCCCC)(C3)C2] |
| AX-53 | 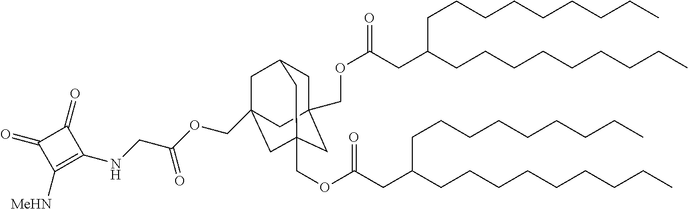 [SMILES: CCCCCCCCCC(CCCCCCCCC)CC(=O)OCC12CC3CC(COC(=O)CNc4c(NC)c(=O)c4=O)(C1)CC(COC(=O)CC(CCCCCCCCC)CCCCCCCCC)(C3)C2] |
| AX-54 | 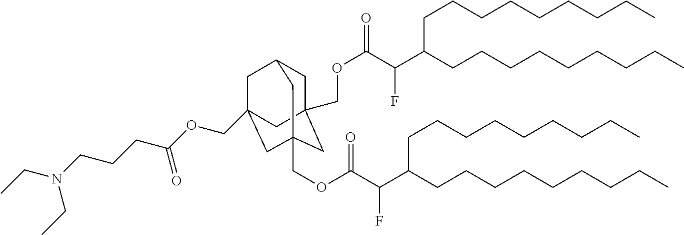 [SMILES: CCCCCCCCCC(CCCCCCCCC)C(F)C(=O)OCC12CC3CC(COC(=O)CCCN(CC)CC)(C1)CC(COC(=O)C(F)C(CCCCCCCCC)CCCCCCCCC)(C3)C2] |
| AX-54a | 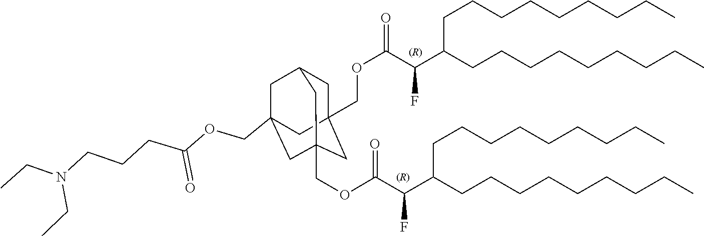 |
| AX-55 | 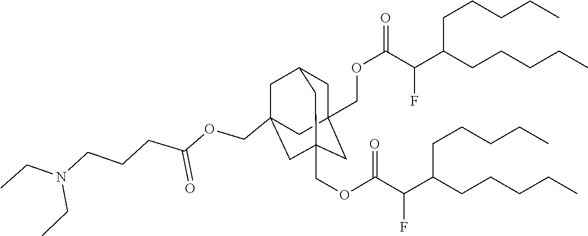 [SMILES: CCCCCC(CCCCC)C(F)C(=O)OCC12CC3CC(COC(=O)CCCCN(CC)CC)(C1)CC(COC(=O)C(F)C(CCCCC)CCCCC)(C3)C2] |
| AX-55a | 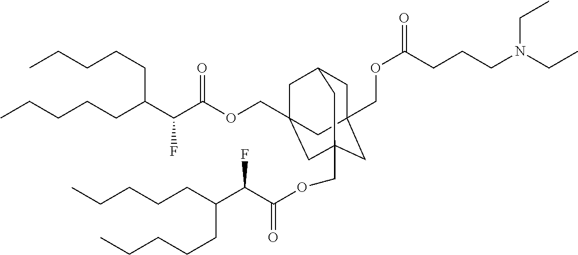 [SMILES: CCCCCC(CCCCC)[C@@H](F)C(=O)OCC12CC3CC(COC(=O)CCCN(CC)CC)(C1)CC(COC(=O)[C@H](F)C(CCCCC)CCCCC)(C3)C2] |
| AX-56 | 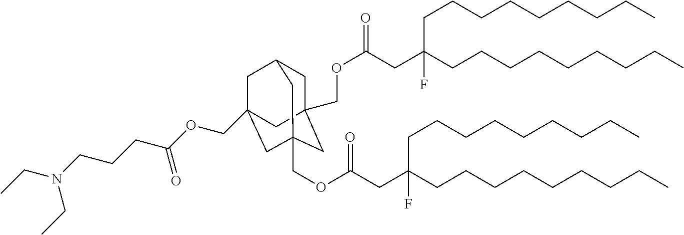 [SMILES: CCCCCCCCCC(F)(CCCCCCCCC)CC(=O)OCC12CC3CC(COC(=O)CCCN(CC)CC)(C1)CC(COC(=O)CC(F)(CCCCCCCCC)CCCCCCCCC)(C3)C2] |
| AX-57 | 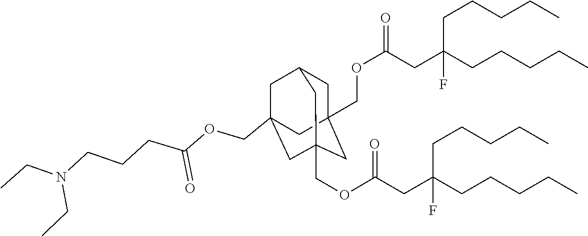 [SMILES: CCCCCC(F)(CCCCC)CC(=O)OCC12CC3CC(COC(=O)CCCCN(CC)CC)(C1)CC(COC(=O)CC(F)(CCCCC)CCCCC)(C3)C2] |
| AX-58 | 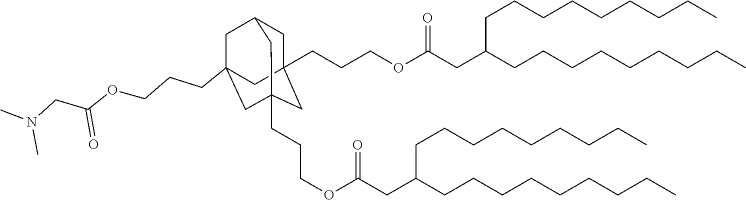 [SMILES: CCCCCCCCCC(CCCCCCCCC)CC(=O)OCCCC12CC3CC(CCCOC(=O)CC(CCCCCCCCC)CCCCCCCCC)(C1)CC(CCCOC(=O)CN(C)C)(C3)C2] |
| AX-59 | 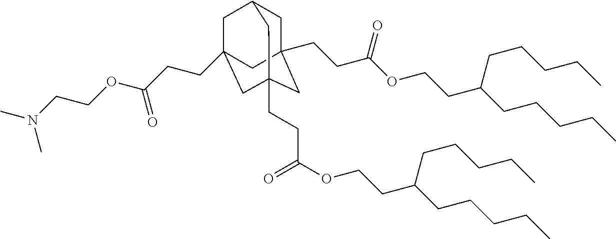 [SMILES: CCCCCC(CCCCC)CCOC(=O)CCC12CC3CC(CCC(=O)OCCC(CCCCC)CCCCC)(C1)CC(CCC(=O)OCCN(C)C)(C3)C2] |
| AX-60 | 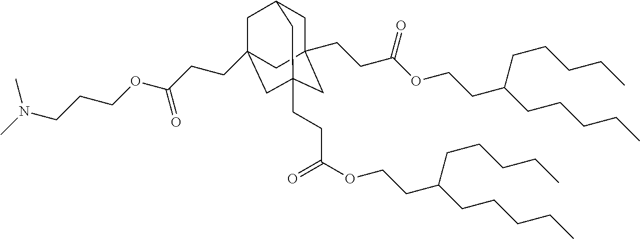 [SMILES: CCCCCC(CCCCC)CCOC(=O)CCC12CC3CC(CCC(=O)OCCCN(C)C)(C1)CC(CCC(=O)OCCC(CCCCC)CCCCC)(C3)C2] |
| AX-61 | 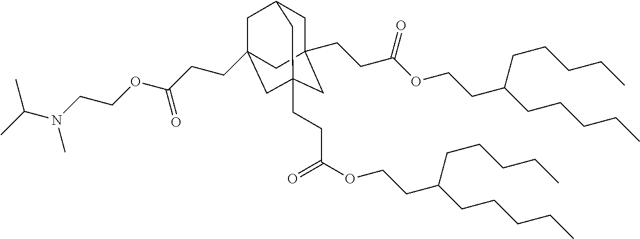 [SMILES: CCCCCC(CCCCC)CCOC(=O)CCC12CC3CC(CCC(=O)OCCC(CCCCC)CCCCC)(C1)CC(CCC(=O)OCCN(C)C(C)C)(C3)C2] |
| AX-62 | 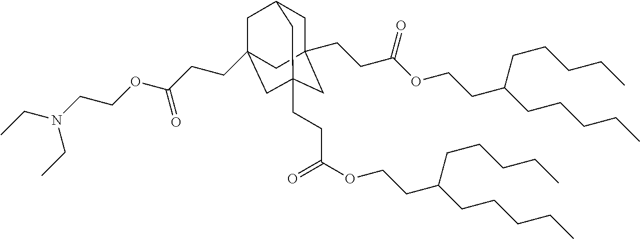 [SMILES: CCCCCC(CCCCC)CCOC(=O)CCC12CC3CC(CCC(=O)OCCC(CCCCC)CCCCC)(C1)CC(CCC(=O)OCCN(CC)CC)(C3)C2] |
| AX-63 | 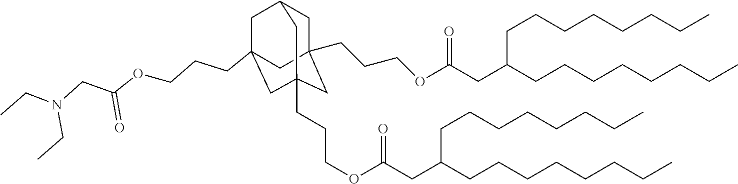 [SMILES: CCCCCCCCC(CCCCCCCC)CC(=O)OCCCC12CC3CC(CCCOC(=O)CC(CCCCCCCC)CCCCCCCC)(C1)CC(CCCOC(=O)CN(CC)CC)(C3)C2] |
| AX-64 | 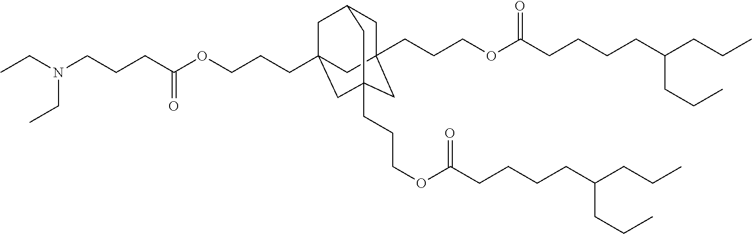 [SMILES: CCCC(CCC)CCCCC(=O)OCCCC12CC3CC(CCCOC(=O)CCCCC(CCC)CCC)(C1)CC(CCCOC(=O)CCCN(CC)CC)(C3)C2] |
| AX-65 | 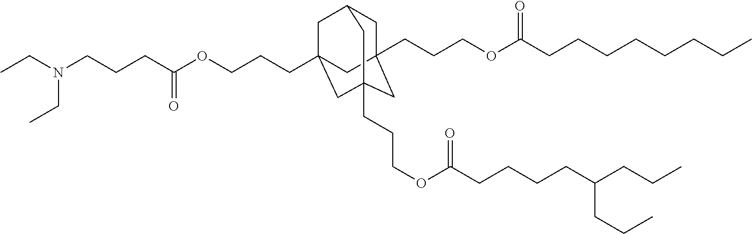 [SMILES: CCCCCCCCC(=O)OCCCC12CC3CC(CCCOC(=O)CCCCC(CCC)CCC)(C1)CC(CCCOC(=O)CCCN(CC)CC)(C3)C2] |
| AX-66 | 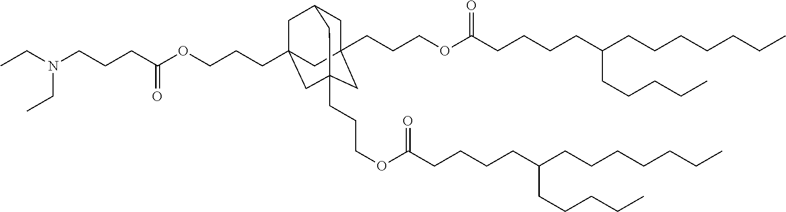 [SMILES: CCCCCCCC(CCCCC)CCCCC(=O)OCCCC12CC3CC(CCCOC(=O)CCCCC(CCCCC)CCCCCCC)(C1)CC(CCCOC(=O)CCCN(CC)CC)(C3)C2] |
| AX-67 | 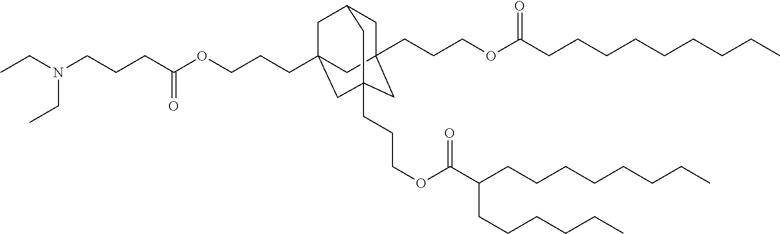 [SMILES: CCCCCCCCCC(=O)OCCCC12CC3CC(CCCOC(=O)CCCN(CC)CC)(C1)CC(CCCOC(=O)C(CCCCCC)CCCCCCCC)(C3)C2] |
| AX-68 | 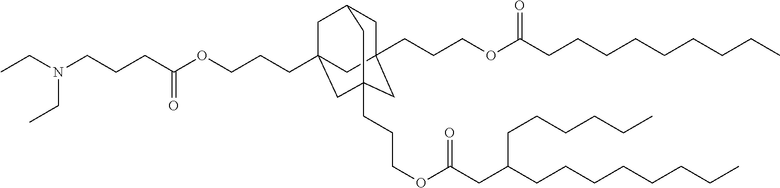 [SMILES: CCCCCCCCCC(=O)OCCCC12CC3CC(CCCOC(=O)CCCN(CC)CC)(C1)CC(CCCOC(=O)CC(CCCCCC)CCCCCCCC)(C3)C2] |
| AX-69 | 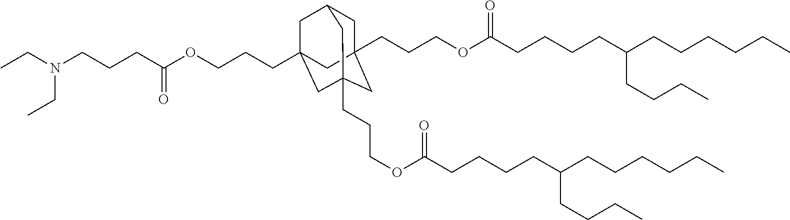 [SMILES: CCCCCCC(CCCC)CCCCC(=O)OCCCC12CC3CC(CCCOC(=O)CCCCC(CCCC)CCCCCC)(C1)CC(CCCOC(=O)CCCN(CC)CC)(C3)C2] |
| AX-70 | 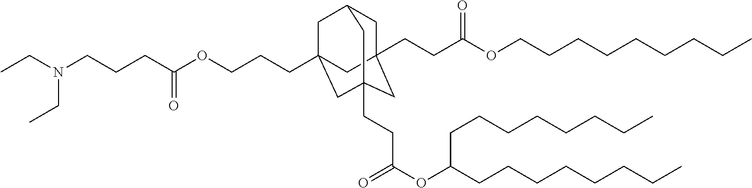 [SMILES: CCCCCCCCCOC(=O)CCC12CC3CC(CCCOC(=O)CCCN(CC)CC)(C1)CC(CCC(=O)OC(CCCCCCCC)CCCCCCCC)(C3)C2] |
| AX-71 | 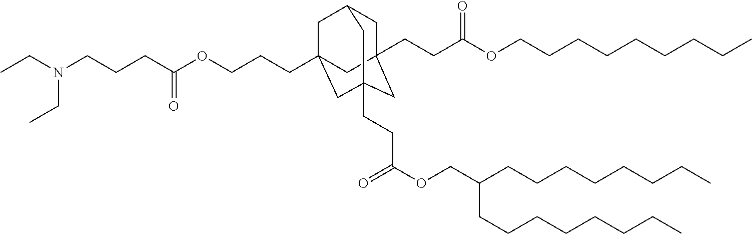 [SMILES: CCCCCCCCCOC(=O)CCC12CC3CC(CCCOC(=O)CCCN(CC)CC)(C1)CC(CCC(=O)OCC(CCCCCCCC)CCCCCCCC)(C3)C2] |
| AX-72 | 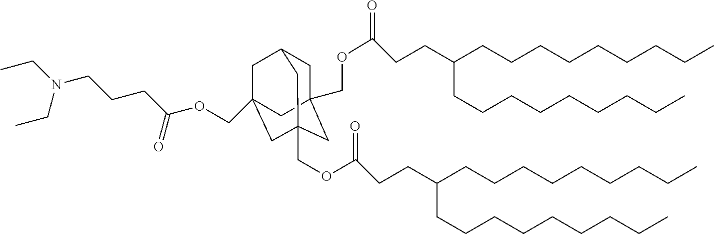 [SMILES: CCCCCCCCCC(CCCCCCCCC)CCC(=O)OCC12CC3CC(COC(=O)CCCN(CC)CC)(C1)CC(COC(=O)CCC(CCCCCCCCC)CCCCCCCCC)(C3)C2] |
| AX-73 | 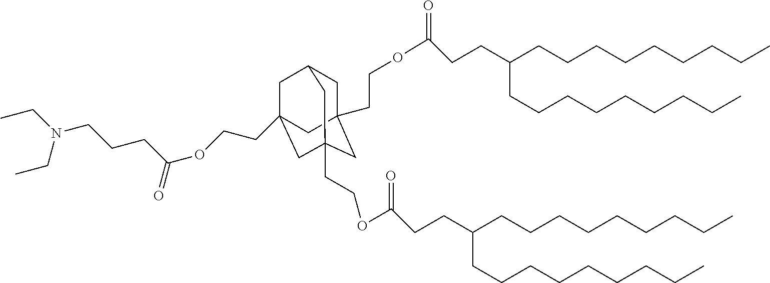 [SMILES: CCCCCCCCCC(CCCCCCCCC)CCC(=O)OCCC12CC3CC(CCOC(=O)CCCN(CC)CC)(C1)CC(CCOC(=O)CCC(CCCCCCCCC)CCCCCCCCC)(C3)C2] |
| AX-74 | 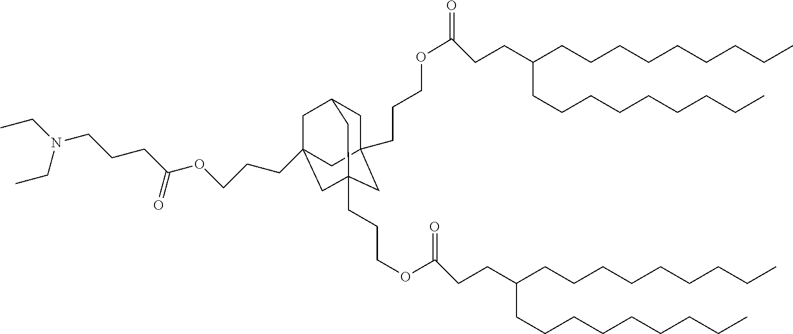 [SMILES: CCCCCCCCCC(CCCCCCCCC)CCC(=O)OCCCC12CC3CC(CCCOC(=O)CCCN(CC)CC)(C1)CC(CCCOC(=O)CCC(CCCCCCCCC)CCCCCCCCC)(C3)C2] |
| AX-75 | 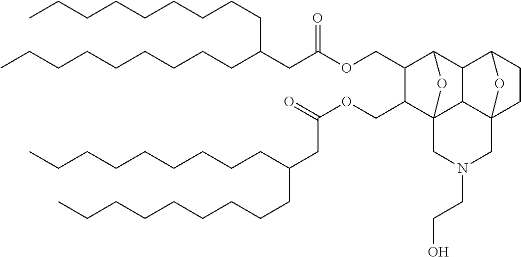 [SMILES: CCCCCCCCCC(CCCCCCCCC)CC(=O)OCC1C2OC3(CN(CCO)CC45CCC(O4)C2C53)C1COC(=O)CC(CCCCCCCCC)CCCCCCCCC] |
| AX-76 | 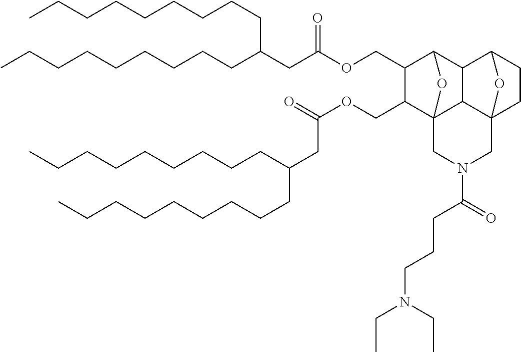 [SMILES: CCCCCCCCCC(CCCCCCCCC)CC(=O)OCC1C2OC3(CN(C(=O)CCCN(CC)CC)CC45CCC(O4)C2C53)C1COC(=O)CC(CCCCCCCCC)CCCCCCCCC] |
| AX-77 | 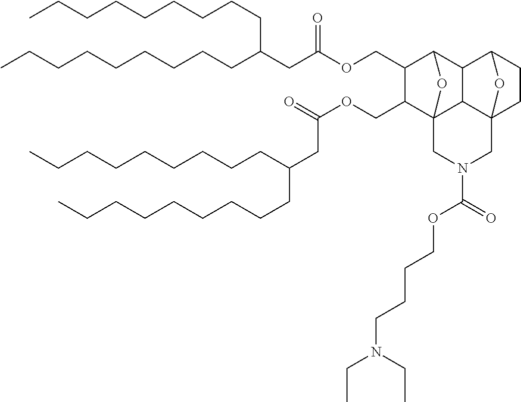 [SMILES: CCCCCCCCCC(CCCCCCCCC)CC(=O)OCC1C2OC3(CN(C(=O)OCCCCN(CC)CC)CC45CCC(O4)C2C53)C1COC(=O)CC(CCCCCCCCC)CCCCCCCCC] |
| AX-78 | 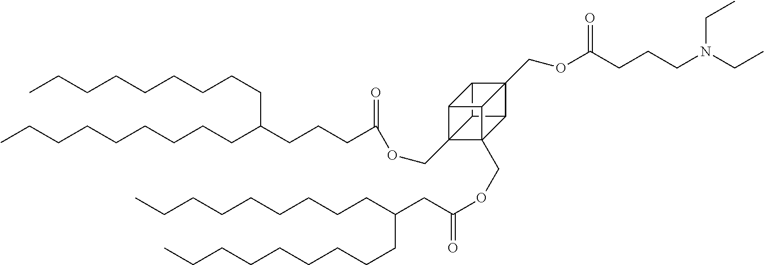 [SMILES: CCCCCCCCCC(CCCCCCCCC)CCCC(=O)OCC12C3C4C1C1C4(COC(=O)CCCN(CC)CC)C3C12COC(=O)CC(CCCCCCCCC)CCCCCCCCC] |
| X-79 | 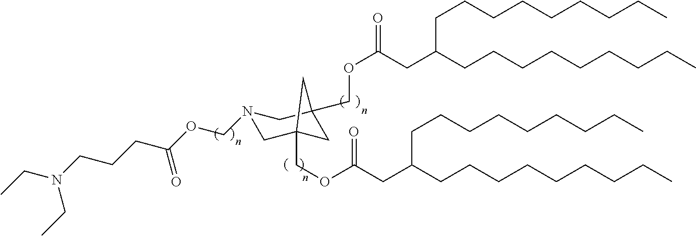 N |
| | n = 1 |
| AX-80 | 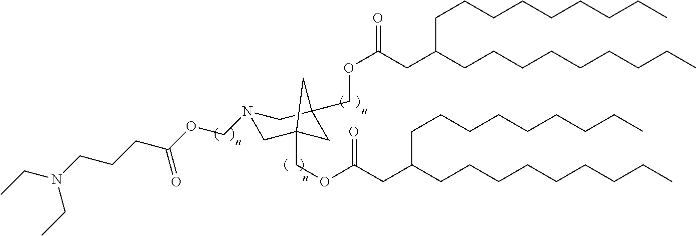 |
| | n = 2 |
| AX-81 | 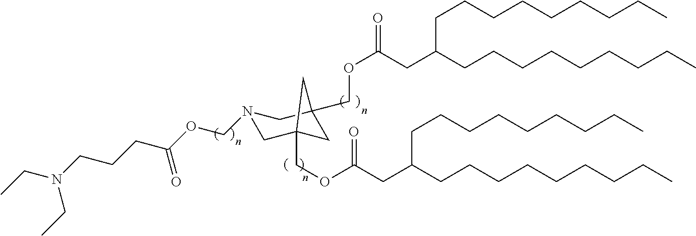 |
| | n = 3 |
| AX-82 | 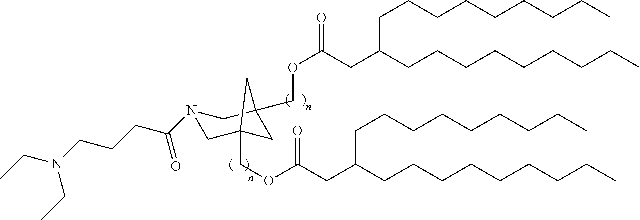 |
| | n = 1 |
| AX-83 | 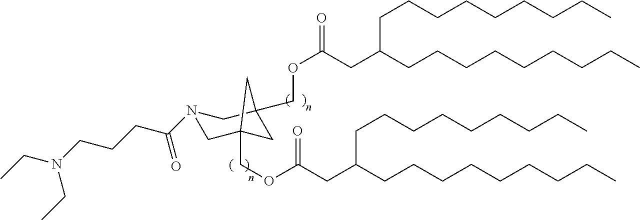 |
| | n = 2 |
| AX-84 | 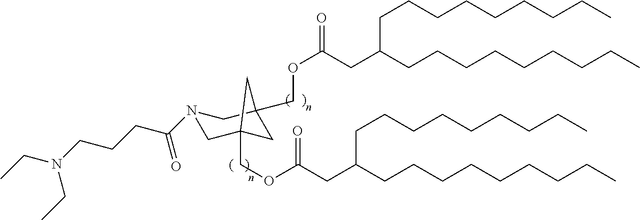 |
| | n = 3 |
| AX-85 | 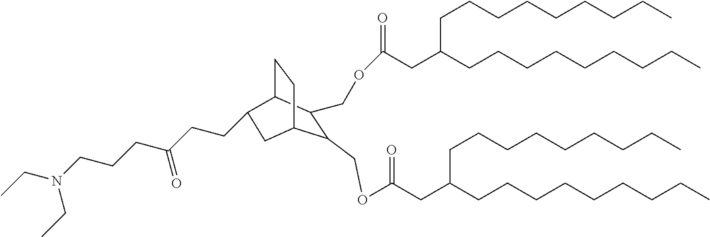 [SMILES: CCCCCCCCCC(CCCCCCCCC)CC(=O)OCC1C2CCC(C(CCC(=O)CCCN(CC)CC)C2)C1COC(=O)CC(CCCCCCCCC)CCCCCCCCC] |
| AX-86 | 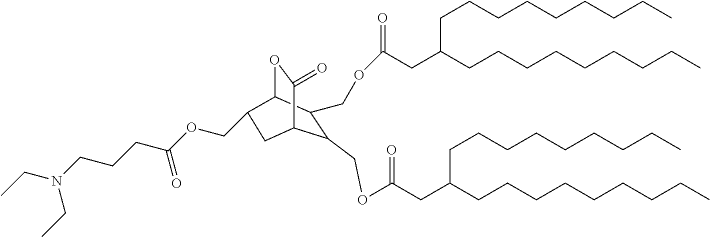 [SMILES: CCCCCCCCCC(CCCCCCCCC)CC(=O)OCC1C2CC(COC(=O)CCCN(CC)CC)C(OC2=O)C1COC(=O)CC(CCCCCCCCC)CCCCCCCCC] |
| AX-87 | 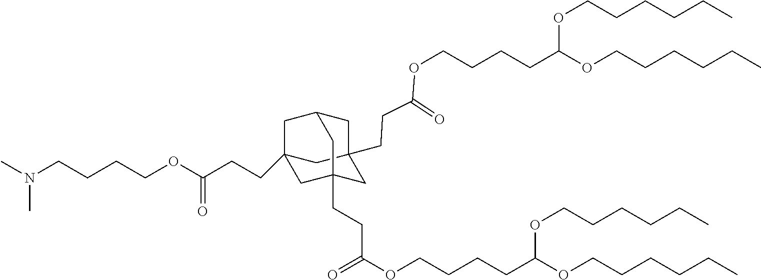 [SMILES: CCCCCCOC(CCCCOC(=O)CCC12CC3CC(CCC(=O)OCCCCC(OCCCCCC)OCCCCCC)(C1)CC(CCC(=O)OCCCCN(C)C)(C3)C2)OCCCCCC] |
| AX-88 | 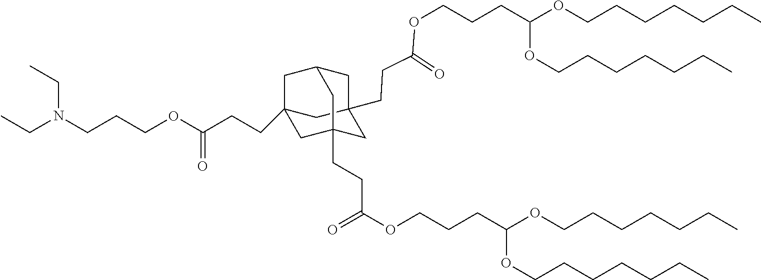 [SMILES: CCCCCCCOC(CCCOC(=O)CCC12CC3CC(CCC(=O)OCCCC(OCCCCCCC)OCCCCCCC)(C1)CC(CCC(=O)OCCCN(CC)CC)(C3)C2)OCCCCCCC] |
| AX-89 | 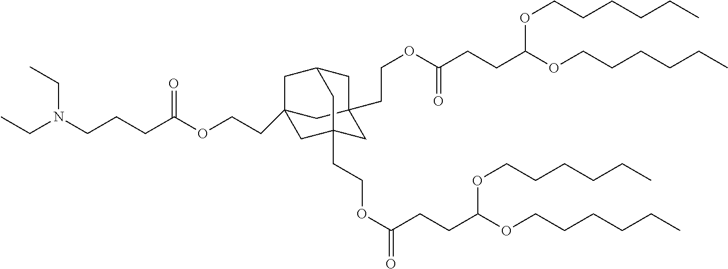 [SMILES: CCCCCCOC(CCC(=O)OCCC12CC3CC(CCOC(=O)CCCN(CC)CC)(C1)CC(CCOC(=O)CCC(OCCCCCC)OCCCCCC)(C3)C2)OCCCCCC] |
| AX-90 | 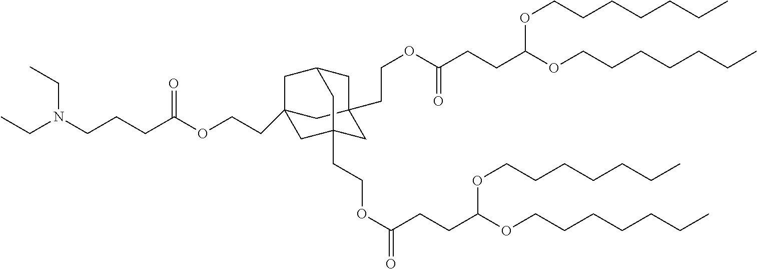 [SMILES: CCCCCCCOC(CCC(=O)OCCC12CC3CC(CCOC(=O)CCCN(CC)CC)(C1)CC(CCOC(=O)CCC(OCCCCCCC)OCCCCCCC)(C3)C2)OCCCCCCC] |
| AX-91 | 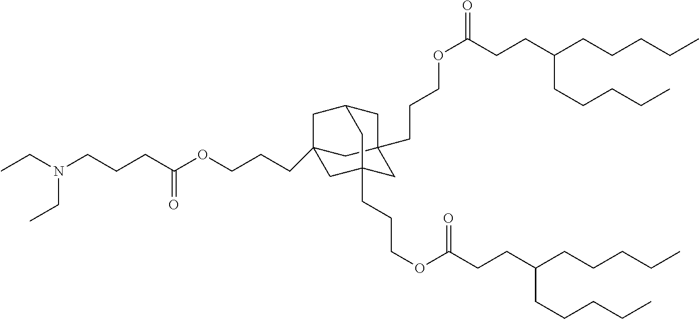 [SMILES: CCCCCC(CCCCC)CCC(=O)OCCCC12CC3CC(CCCOC(=O)CCCN(CC)CC)(C1)CC(CCCOC(=O)CCC(CCCCC)CCCCC)(C3)C2] |
| AX-92 | 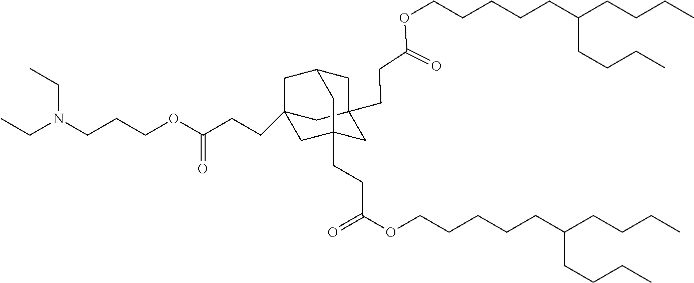 [SMILES: CCCCC(CCCC)CCCCCOC(=O)CCC12CC3CC(CCC(=O)OCCCCCC(CCCC)CCCC)(C1)CC(CCC(=O)OCCCN(CC)CC)(C3)C2] |
| AX-93 | 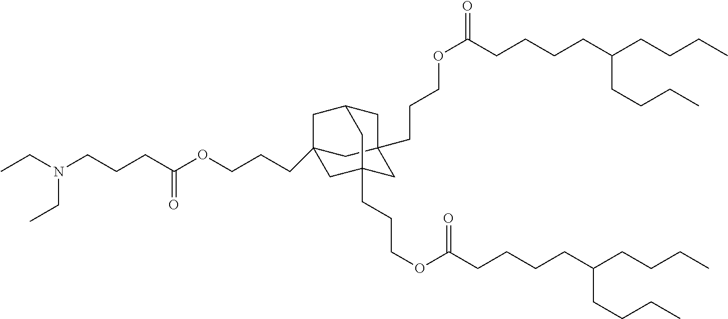 [SMILES: CCCCC(CCCC)CCCCC(=O)OCCCC12CC3CC(CCCOC(=O)CCCCC(CCCC)CCCC)(C1)CC(CCCOC(=O)CCCN(CC)CC)(C3)C2] |
| AX-94 | 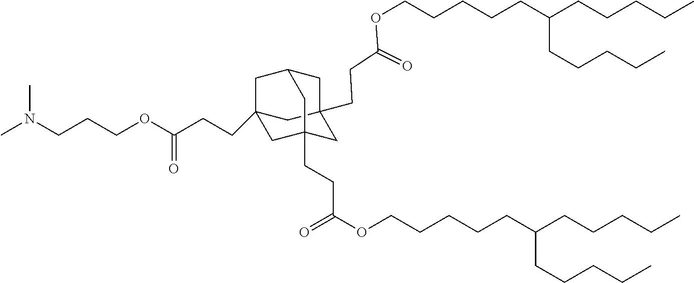 [SMILES: CCCCCC(CCCCC)CCCCCOC(=O)CCC12CC3CC(CCC(=O)OCCCCCC(CCCCC)CCCCC)(C1)CC(CCC(=O)OCCCN(C)C)(C3)C2] |
| AX-95 | 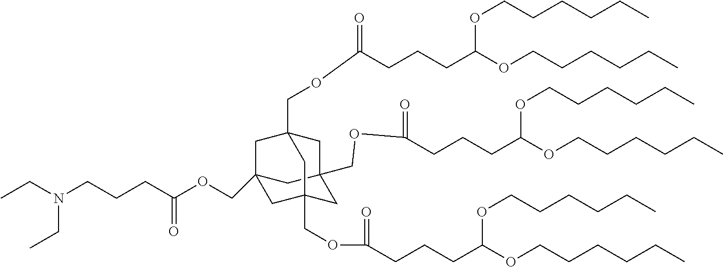 [SMILES: CCCCCCOC(CCCC(=O)OCC12CC3(COC(=O)CCCC(OCCCCCC)OCCCCCC)CC(COC(=O)CCCC(OCCCCCC)OCCCCCC)(C1)CC(COC(=O)CCCN(CC)CC)(C2)C3)OCCCCCC] |
| AX-96 | 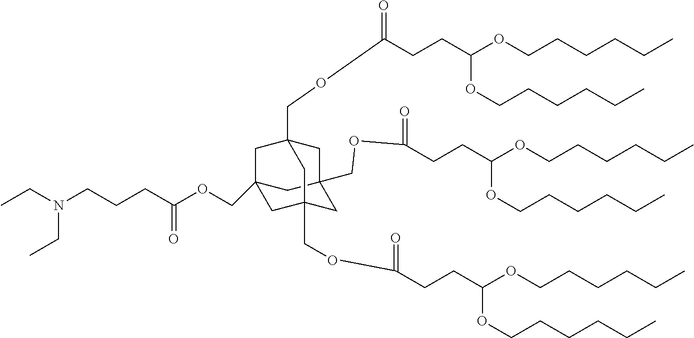 [SMILES: CCCCCCOC(CCC(=O)OCC12CC3(COC(=O)CCCN(CC)CC)CC(COC(=O)CCC(OCCCCCC)OCCCCCC)(C1)CC(COC(=O)CCC(OCCCCCC)OCCCCCC)(C3)C2)OCCCCCC] |
| AX-97 | 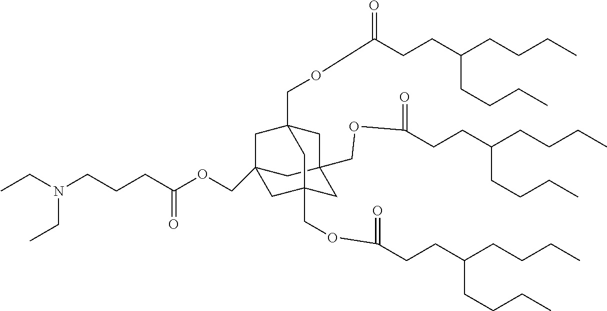 [SMILES: CCCCC(CCCC)CCC(=O)OCC12CC3(COC(=O)CCCN(CC)CC)CC(COC(=O)CCC(CCCC)CCCC)(C1)CC(COC(=O)CCC(CCCC)CCCC)(C3)C2] |
| AX-98 | 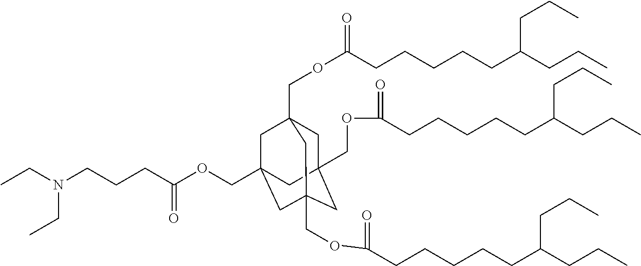 [SMILES: CCCC(CCC)CCCCCC(=O)OCC12CC3(COC(=O)CCCCCC(CCC)CCC)CC(COC(=O)CCCCCC(CCC)CCC)(C1)CC(COC(=O)CCCN(CC)CC)(C2)C3] |
| AX-99 | 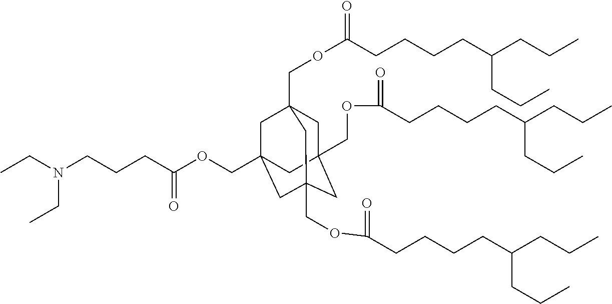 [SMILES: CCCC(CCC)CCCCC(=O)OCC12CC3(COC(=O)CCCCC(CCC)CCC)CC(COC(=O)CCCCC(CCC)CCC)(C1)CC(COC(=O)CCCN(CC)CC)(C2)C3] |
| AX-100 | 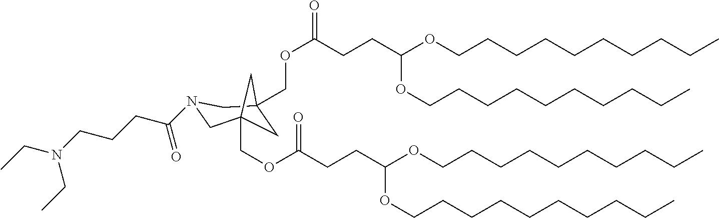 [SMILES: CCCCCCCCCCOC(CCC(=O)OCC12CN(C(=O)CCCN(CC)CC)CC(COC(=O)CCC(OCCCCCCCCCC)OCCCCCCCCCC)(C1)C2)OCCCCCCCCCC] |
| AX-101 | 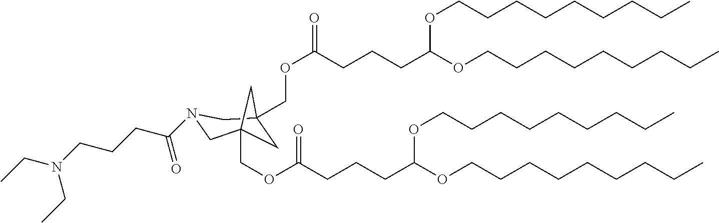 [SMILES: CCCCCCCCCOC(CCCC(=O)OCC12CN(C(=O)CCCN(CC)CC)CC(COC(=O)CCCC(OCCCCCCCCC)OCCCCCCCCC)(C1)C2)OCCCCCCCCC] |
| AX-102 | 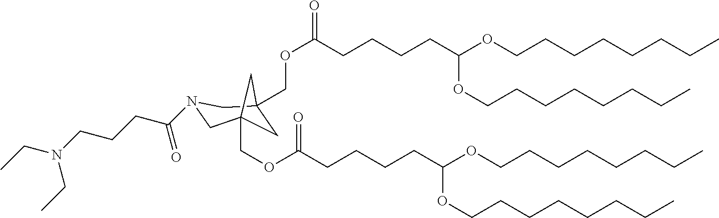 [SMILES: CCCCCCCCOC(CCCCC(=O)OCC12CN(C(=O)CCCN(CC)CC)CC(COC(=O)CCCCC(OCCCCCCCC)OCCCCCCCC)(C1)C2)OCCCCCCCC] |
| AX-103 | 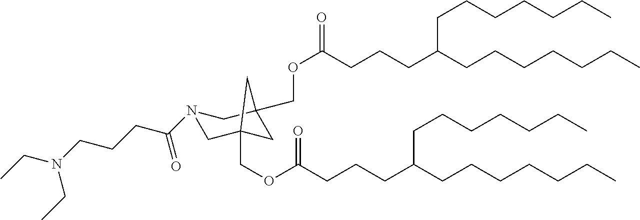 [SMILES: CCCCCCCC(CCCCCCC)CCCC(=O)OCC12CN(C(=O)CCCN(CC)CC)CC(COC(=O)CCCC(CCCCCCC)CCCCCCC)(C1)C2] |
| AX-104 | 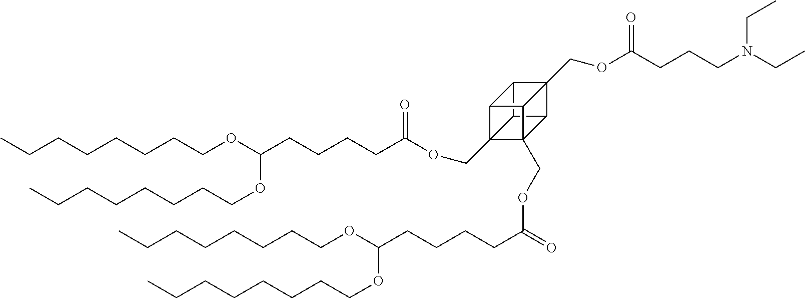 [SMILES: CCCCCCCCOC(CCCCC(=O)OCC12C3C4C1C1C4(COC(=O)CCCN(CC)CC)C3C12COC(=O)CCCCC(OCCCCCCCC)OCCCCCCCC)OCCCCCCCC] |
| AX-105 | 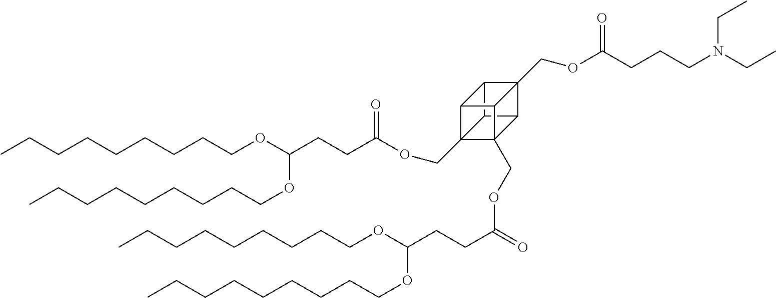 [SMILES: CCCCCCCCCOC(CCC(=O)OCC12C3C4C1C1C4(COC(=O)CCCN(CC)CC)C3C12COC(=O)CCC(OCCCCCCCCC)OCCCCCCCCC)OCCCCCCCCC] |
| AX-106 | 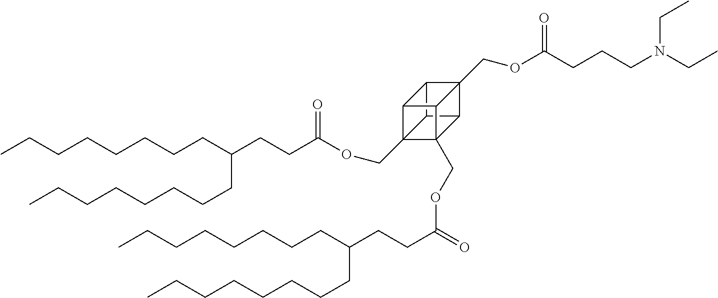 [SMILES: CCCCCCCCC(CCCCCCCC)CCC(=O)OCC12C3C4C1C1C4(COC(=O)CCCN(CC)CC)C3C12COC(=O)CCC(CCCCCCCC)CCCCCCCC] |
| AX-107 | 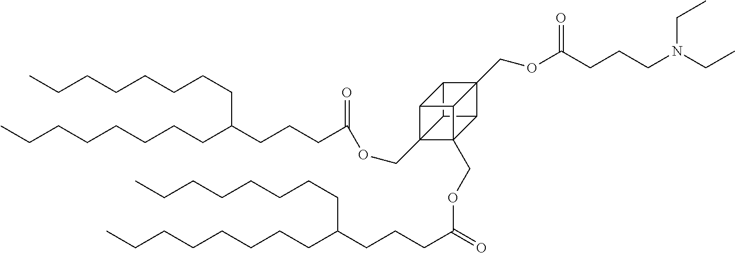 [SMILES: CCCCCCCCC(CCCCCCCC)CCCC(=O)OCC12C3C4C1C1C4(COC(=O)CCCN(CC)CC)C3C12COC(=O)CCCC(CCCCCCCC)CCCCCCCC] |
| AX-108 | 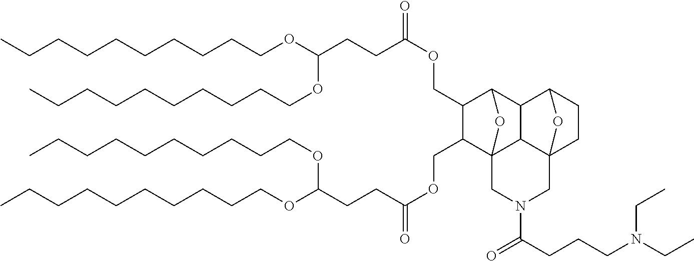 [SMILES: CCCCCCCCCCOC(CCC(=O)OCC1C2OC3(CN(C(=O)CCCN(CC)CC)CC45CCC(O4)C2C53)C1COC(=O)CCC(OCCCCCCCCCC)OCCCCCCCCCC)OCCCCCCCCCC] |
| AX-109 | 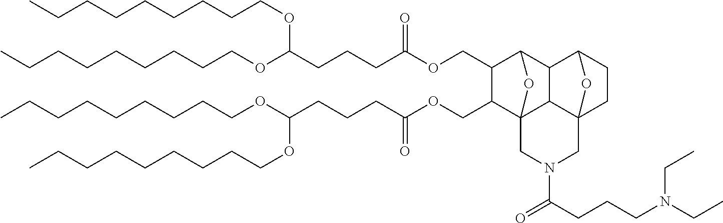 [SMILES: CCCCCCCCCOC(CCCC(=O)OCC1C2OC3(CN(C(=O)CCCN(CC)CC)CC45CCC(O4)C2C53)C1COC(=O)CCCC(OCCCCCCCCC)OCCCCCCCCC)OCCCCCCCCC] |
| AX-110 | 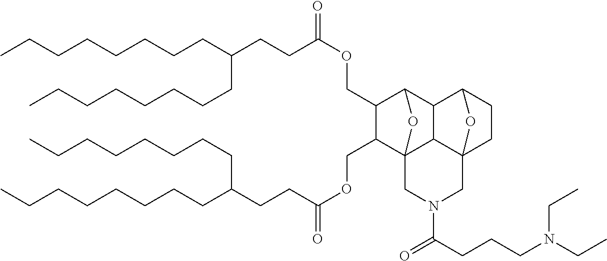 [SMILES: CCCCCCCCC(CCCCCCCC)CCC(=O)OCC1C2OC3(CN(C(=O)CCCN(CC)CC)CC45CCC(O4)C2C53)C1COC(=O)CCC(CCCCCCCC)CCCCCCCC] |
| AX-111 | 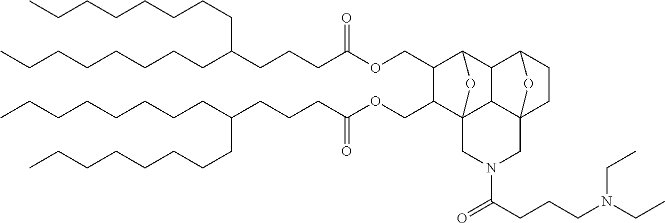 [SMILES: CCCCCCCCC(CCCCCCCC)CCCC(=O)OCC1C2OC3(CN(C(=O)CCCN(CC)CC)CC45CCC(O4)C2C53)C1COC(=O)CCCC(CCCCCCCC)CCCCCCCC] |
| AX-112 | 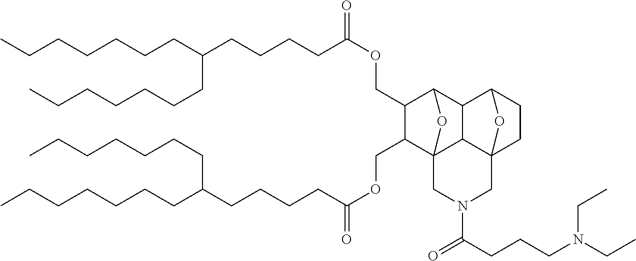 [SMILES: CCCCCCCC(CCCCCCC)CCCCC(=O)OCC1C2OC3(CN(C(=O)CCCN(CC)CC)CC45CCC(O4)C2C53)C1COC(=O)CCCCC(CCCCCCC)CCCCCCC] |
| AX-113 | 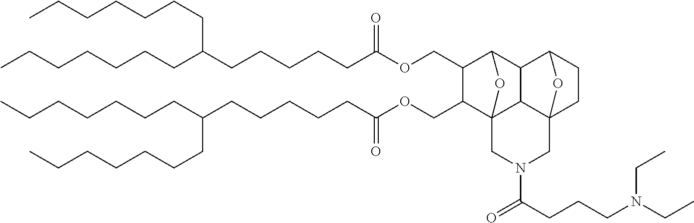 [SMILES: CCCCCCCC(CCCCCCC)CCCCCC(=O)OCC1C2OC3(CN(C(=O)CCCN(CC)CC)CC45CCC(O4)C2C53)C1COC(=O)CCCCCC(CCCCCCC)CCCCCCC] |
| AX-114 | 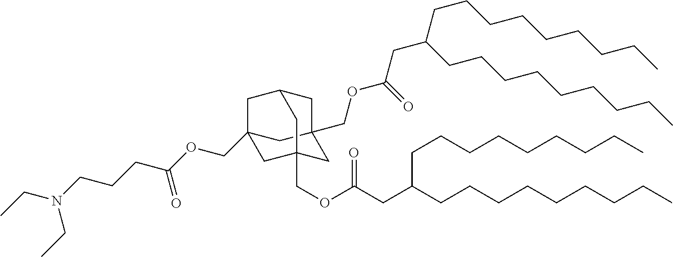 [SMILES: CCCCCCCCCC(CCCCCCCCC)CC(=O)OCC12CC3CC(COC(=O)CCCN(CC)CC)(C1)CC(COC(=O)CC(CCCCCCCCC)CCCCCCCCC)(C3)C2] |
| AX-115 | 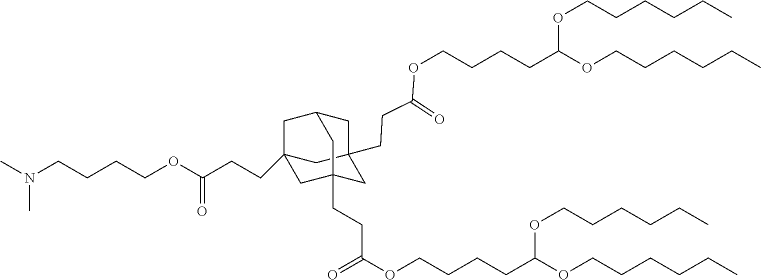 [SMILES: CCCCCCOC(CCCCOC(=O)CCC12CC3CC(CCC(=O)OCCCCC(OCCCCCC)OCCCCCC)(C1)CC(CCC(=O)OCCCCN(C)C)(C3)C2)OCCCCCC] |
| AX-116 | 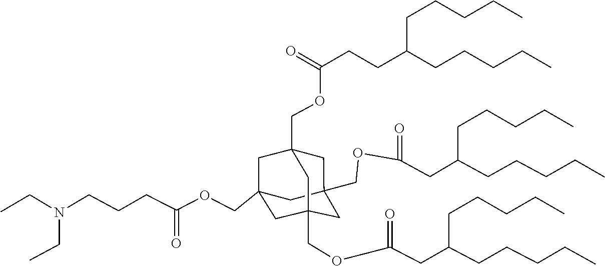 [SMILES: CCCCCC(CCCCC)CCC(=O)OCC12CC3(COC(=O)CCCN(CC)CC)CC(COC(=O)CC(CCCCC)CCCCC)(C1)CC(COC(=O)CC(CCCCC)CCCCC)(C3)C2] |
| AX-117 | 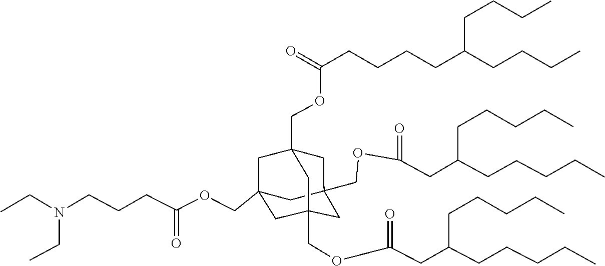 [SMILES: CCCCCC(CCCCC)CC(=O)OCC12CC3(COC(=O)CCCCC(CCCC)CCCC)CC(COC(=O)CCCN(CC)CC)(C1)CC(COC(=O)CC(CCCCC)CCCCC)(C3)C2] |
| AX-118 | 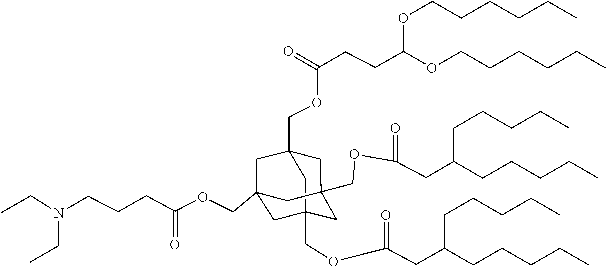 [SMILES: CCCCCCOC(CCC(=O)OCC12CC3(COC(=O)CCCN(CC)CC)CC(COC(=O)CC(CCCCC)CCCCC)(C1)CC(COC(=O)CC(CCCCC)CCCCC)(C3)C2)OCCCCCC] |
| AX-119 | 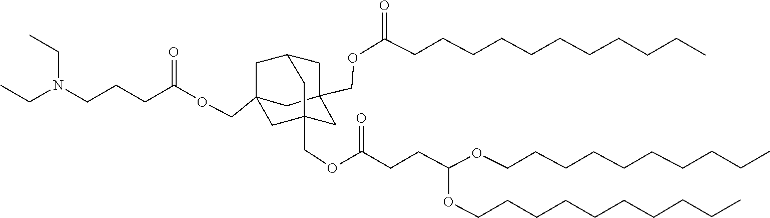 [SMILES: CCCCCCCCCCCC(=O)OCC12CC3CC(COC(=O)CCCN(CC)CC)(C1)CC(COC(=O)CCC(OCCCCCCCCCC)OCCCCCCCCCC)(C3)C2] |
| AX-120 | 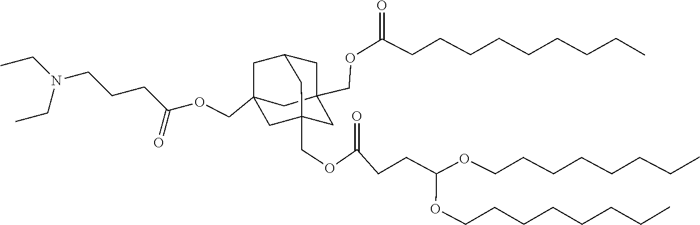 [SMILES: CCCCCCCCCC(=O)OCC12CC3CC(COC(=O)CCCN(CC)CC)(C1)CC(COC(=O)CCC(OCCCCCCCC)OCCCCCCCC)(C3)C2] |
| AX-121 | 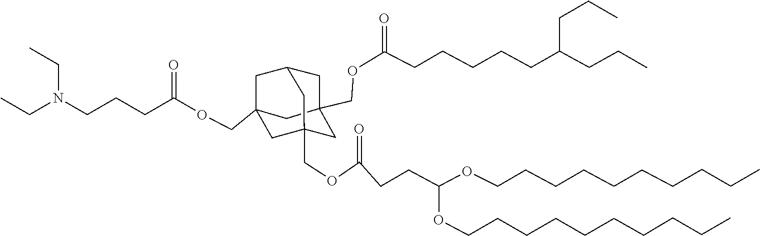 [SMILES: CCCCCCCCCCOC(CCC(=O)OCC12CC3CC(COC(=O)CCCCCC(CCC)CCC)(CC(COC(=O)CCCN(CC)CC)(C3)C1)C2)OCCCCCCCCCC] |
| AX-122 | 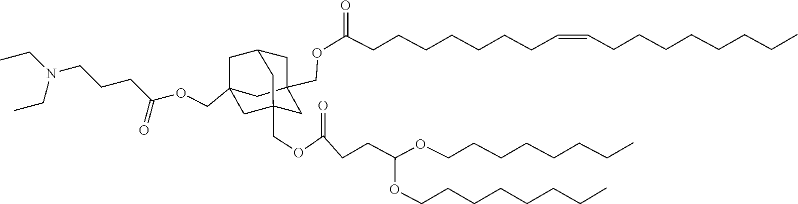 [SMILES: CCCCCCCC/C=C\CCCCCCCC(=O)OCC12CC3CC(COC(=O)CCCN(CC)CC)(C1)CC(COC(=O)CCC(OCCCCCCCC)OCCCCCCCC)(C3)C2] |
| AX-123 | 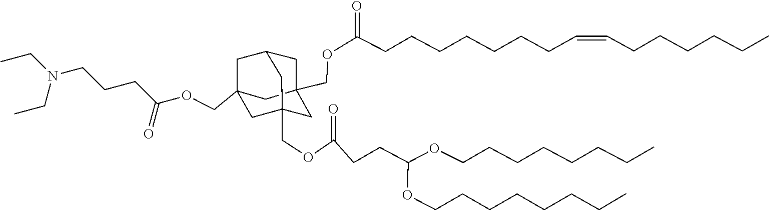 [SMILES: CCCCCC/C=C\CCCCCCCC(=O)OCC12CC3CC(COC(=O)CCCN(CC)CC)(C1)CC(COC(=O)CCC(OCCCCCCCC)OCCCCCCCC)(C3)C2] |
| AX-124 | 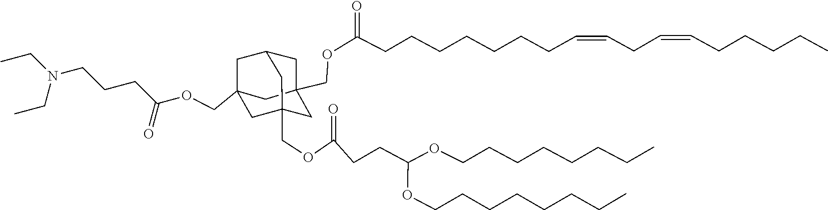 [SMILES: CCCCC/C=C\C/C=C\CCCCCCCC(=O)OCC12CC3CC(COC(=O)CCCN(CC)CC)(C1)CC(COC(=O)CCC(OCCCCCCCC)OCCCCCCCC)(C3)C2] |
| AX-125 | 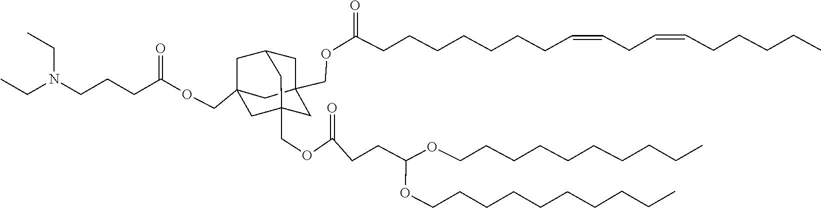 [SMILES: CCCCC/C=C\C/C=C\CCCCCCCC(=O)OCC12CC3CC(COC(=O)CCCN(CC)CC)(C1)CC(COC(=O)CCC(OCCCCCCCCCC)OCCCCCCCCCC)(C3)C2] |
| AX-126 | 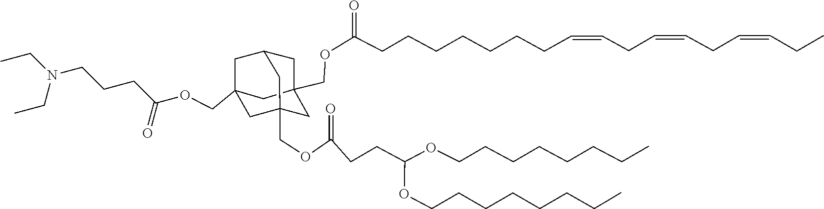 [SMILES: CC/C=C\C/C=C\C/C=C\CCCCCCCC(=O)OCC12CC3CC(COC(=O)CCCN(CC)CC)(C1)CC(COC(=O)CCC(OCCCCCCCC)OCCCCCCCC)(C3)C2] |
| AX-127 | 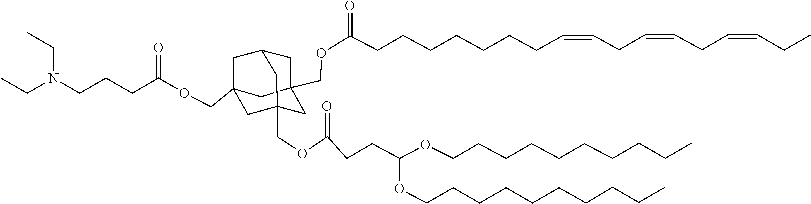 [SMILES: CC/C=C\C/C=C\C/C=C\CCCCCCCC(=O)OCC12CC3CC(COC(=O)CCCN(CC)CC)(C1)CC(COC(=O)CCC(OCCCCCCCCCC)OCCCCCCCCCC)(C3)C2] |
| AX-128 | 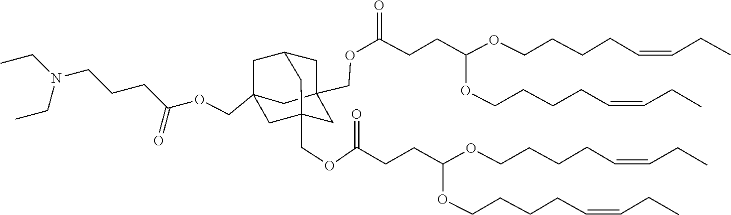 [SMILES: CC/C=C\CCCCOC(CCC(=O)OCC12CC3CC(COC(=O)CCCN(CC)CC)(C1)CC(COC(=O)CCC(OCCCC/C=C\CC)OCCCC/C=C\CC)(C3)C2)OCCCC/C=C\CC] |
| AX-129 | 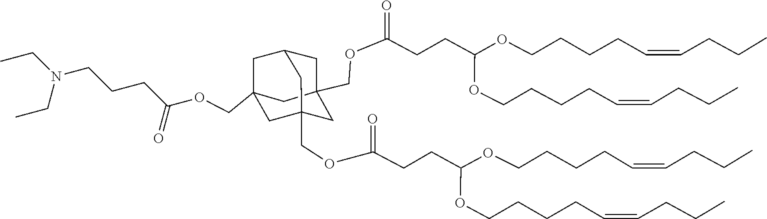 [SMILES: CCC/C=C\CCCCOC(CCC(=O)OCC12CC3CC(COC(=O)CCCN(CC)CC)(C1)CC(COC(=O)CCC(OCCCC/C=C\CCC)OCCCC/C=C\CCC)(C3)C2)OCCCC/C=C\CCC] |
| AX-130 | 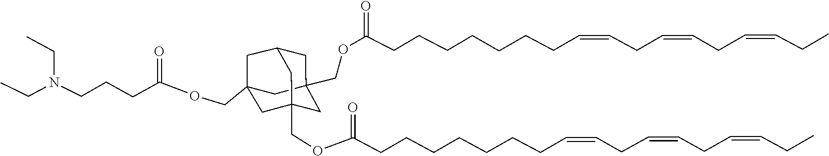 [SMILES: CC/C=C\C/C=C\C/C=C\CCCCCCCC(=O)OCC12CC3CC(COC(=O)CCCCCCC/C=C\C/C=C\C/C=C\CC)(C1)CC(COC(=O)CCCN(CC)CC)(C3)C2] |
| AX-131 | 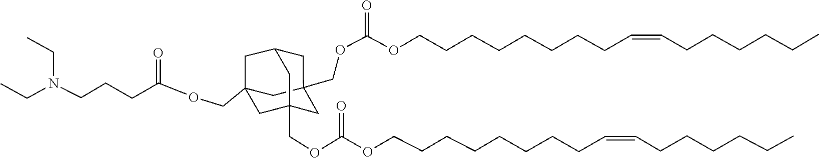 [SMILES: CCCCCC/C=C\CCCCCCCCOC(=O)OCC12CC3CC(COC(=O)CCCN(CC)CC)(C1)CC(COC(=O)OCCCCCCCC/C=C\CCCCCC)(C3)C2] |
| AX-132 | 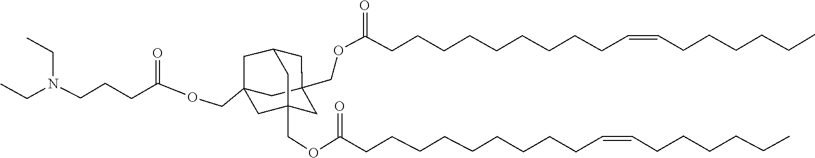 [SMILES: CCCCCC/C=C\CCCCCCCCCC(=O)OCC12CC3CC(COC(=O)CCCCCCCCC/C=C\CCCCCC)(C1)CC(COC(=O)CCCN(CC)CC)(C3)C2] |
| AX-133 | 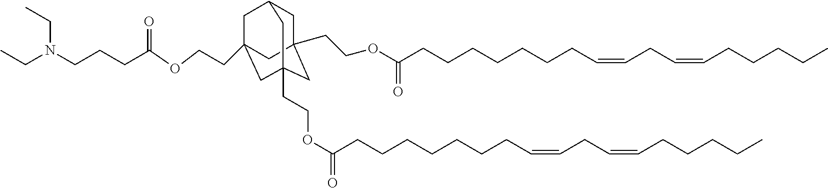 [SMILES: CCCCC/C=C\C/C=C\CCCCCCCC(=O)OCCC12CC3CC(CCOC(=O)CCCCCCC/C=C\C/C=C\CCCCC)(C1)CC(CCOC(=O)CCCN(CC)CC)(C3)C2] |
| AX-134 | 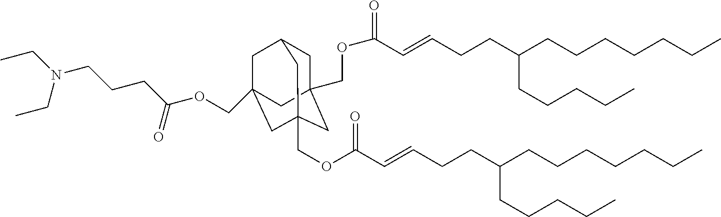 [SMILES: CCCCCCCC(CC/C=C/C(=O)OCC12CC3CC(COC(=O)/C=C/CCC(CCCCC)CCCCCCC)(C1)CC(COC(=O)CCCN(CC)CC)(C3)C2)CCCCC] |
| AX-135 | 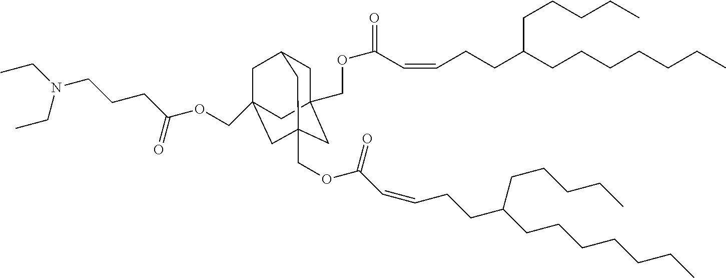 [SMILES: CCCCCCCC(CC/C=C\C(=O)OCC12CC3CC(COC(=O)/C=C\CCC(CCCCC)CCCCCCC)(C1)CC(COC(=O)CCCN(CC)CC)(C3)C2)CCCCC] |
| AX-136 | 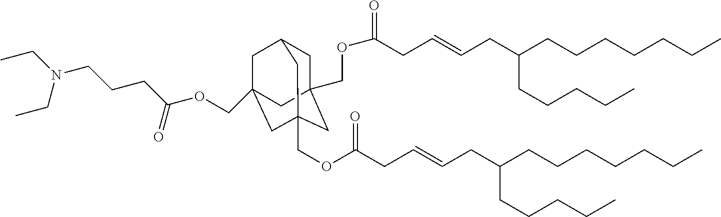 [SMILES: CCCCCCCC(C/C=C/CC(=O)OCC12CC3CC(COC(=O)C/C=C/CC(CCCCC)CCCCCCC)(C1)CC(COC(=O)CCCN(CC)CC)(C3)C2)CCCCC] |
| AX-137 | 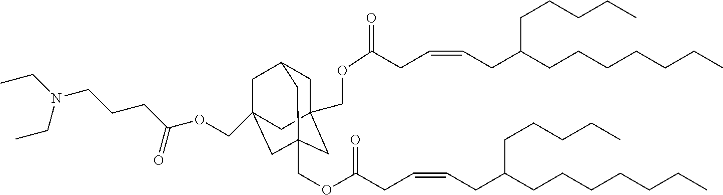 [SMILES: CCCCCCCC(C/C=C\CC(=O)OCC12CC3CC(COC(=O)C/C=C\CC(CCCCC)CCCCCCC)(C1)CC(COC(=O)CCCN(CC)CC)(C3)C2)CCCCC] |
| AX-138 | 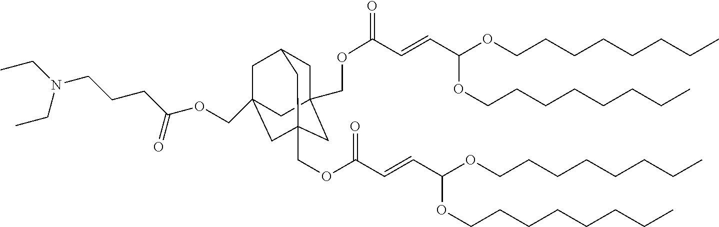 [SMILES: CCCCCCCCOC(/C=C/C(=O)OCC12CC3CC(COC(=O)/C=C/C(OCCCCCCCC)OCCCCCCCC)(C1)CC(COC(=O)CCCN(CC)CC)(C3)C2)OCCCCCCCC] |
| AX-139 | 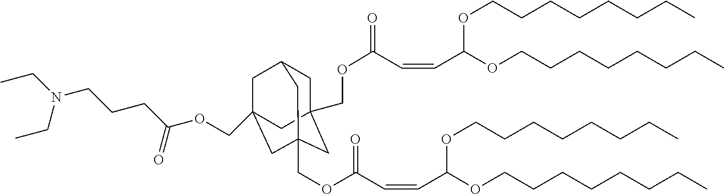 [SMILES: CCCCCCCCOC(/C=C\C(=O)OCC12CC3CC(COC(=O)/C=C\C(OCCCCCCCC)OCCCCCCCC)(C1)CC(COC(=O)CCCN(CC)CC)(C3)C2)OCCCCCCCC] |
| AX-140 | 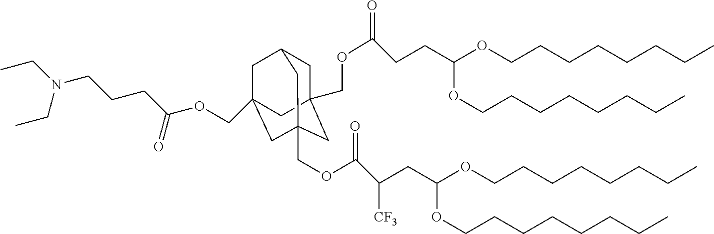 [SMILES: CCCCCCCCOC(CCC(=O)OCC12CC3CC(COC(=O)CCCN(CC)CC)(C1)CC(COC(=O)C(CC(OCCCCCCCC)OCCCCCCCC)C(F)(F)F)(C3)C2)OCCCCCCCC] |
| AX-141 | 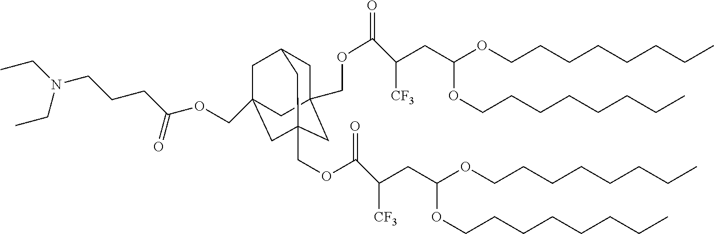 [SMILES: CCCCCCCCOC(CC(C(=O)OCC12CC3CC(COC(=O)CCCN(CC)CC)(C1)CC(COC(=O)C(CC(OCCCCCCCC)OCCCCCCCC)C(F)(F)F)(C3)C2)C(F)(F)F)OCCCCCCCC] |
| AX-142 | 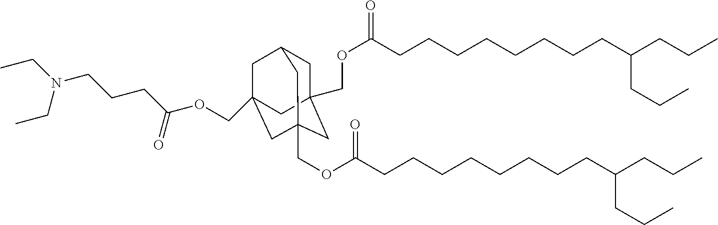 [SMILES: CCCC(CCC)CCCCCCCCC(=O)OCC12CC3CC(COC(=O)CCCCCCCCC(CCC)CCC)(C1)CC(COC(=O)CCCN(CC)CC)(C3)C2] |
| AX-143 | 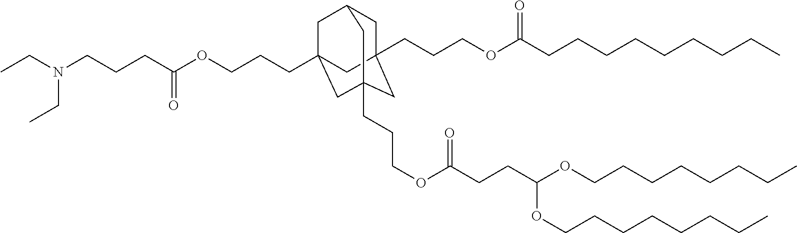 [SMILES: CCCCCCCCCC(=O)OCCCC12CC3CC(CCCOC(=O)CCCN(CC)CC)(C1)CC(CCCOC(=O)CCC(OCCCCCCCC)OCCCCCCCC)(C3)C2] |
| AX-144 | 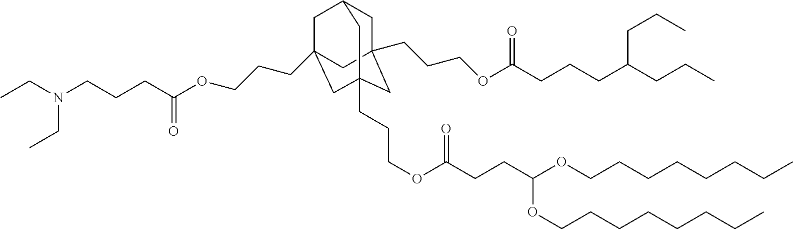 [SMILES: CCCCCCCCOC(CCC(=O)OCCCC12CC3CC(CCCOC(=O)CCCC(CCC)CCC)(CC(CCCOC(=O)CCCN(CC)CC)(C3)C1)C2)OCCCCCCCC] |
| AX-145 | 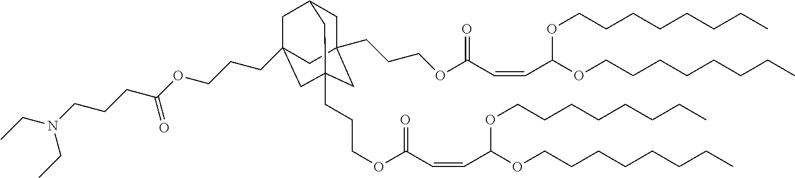 [SMILES: CCCCCCCCOC(/C=C\C(=O)OCCCC12CC3CC(CCCOC(=O)/C=C\C(OCCCCCCCC)OCCCCCCCC)(C1)CC(CCCOC(=O)CCCN(CC)CC)(C3)C2)OCCCCCCCC] |
| AX-146 | 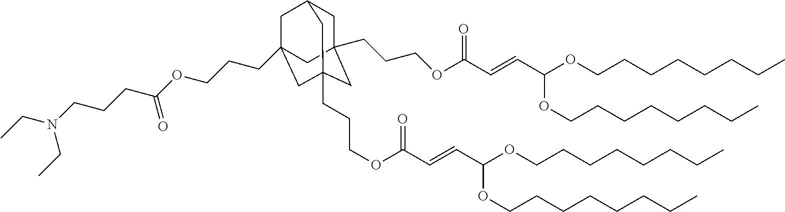 [SMILES: CCCCCCCCOC(/C=C/C(=O)OCCCC12CC3CC(CCCOC(=O)/C=C/C(OCCCCCCCC)OCCCCCCCC)(C1)CC(CCCOC(=O)CCCN(CC)CC)(C3)C2)OCCCCCCCC] |
| AX-147 | 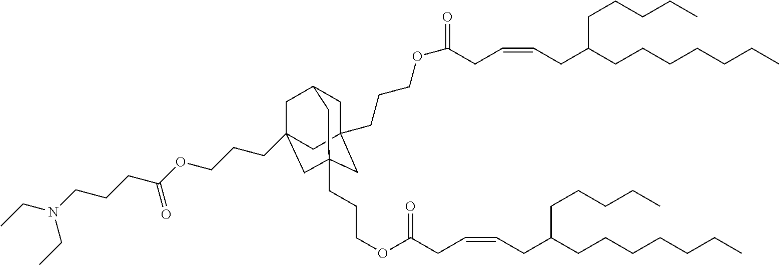 [SMILES: CCCCCCCC(C/C=C\CC(=O)OCCCC12CC3CC(CCCOC(=O)C/C=C\CC(CCCCC)CCCCCCC)(C1)CC(CCCOC(=O)CCCN(CC)CC)(C3)C2)CCCCC] |
| AX-148 | 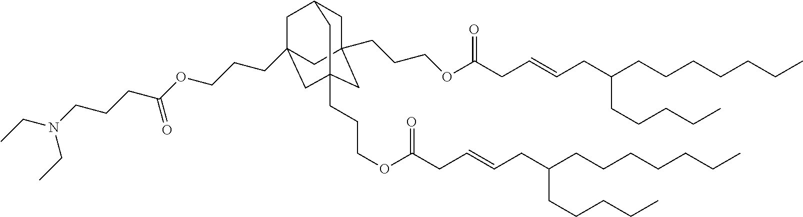 [SMILES: CCCCCCCC(C/C=C/CC(=O)OCCCC12CC3CC(CCCOC(=O)C/C=C/CC(CCCCC)CCCCCCC)(C1)CC(CCCOC(=O)CCCN(CC)CC)(C3)C2)CCCCC] |
| AX-149 | 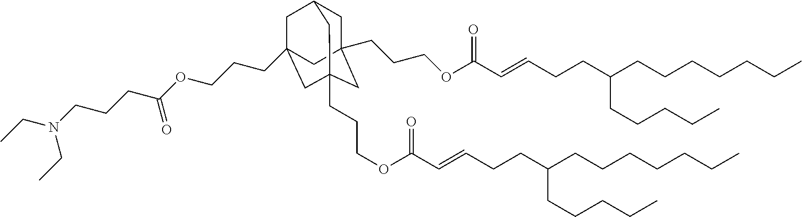 [SMILES: CCCCCCCC(CC/C=C/C(=O)OCCCC12CC3CC(CCCOC(=O)/C=C/CCC(CCCCC)CCCCCCC)(C1)CC(CCCOC(=O)CCCN(CC)CC)(C3)C2)CCCCC] |
| AX-150 | 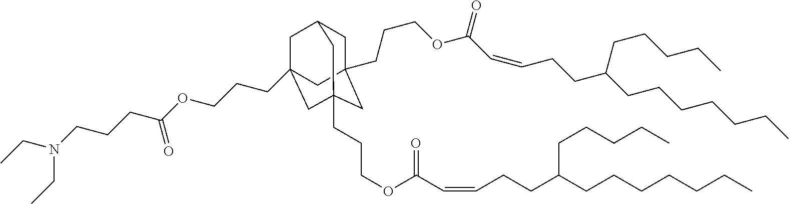 [SMILES: CCCCCCCC(CC/C=C\C(=O)OCCCC12CC3CC(CCCOC(=O)/C=C\CCC(CCCCC)CCCCCCC)(C1)CC(CCCOC(=O)CCCN(CC)CC)(C3)C2)CCCCC] |
| AX-151 | 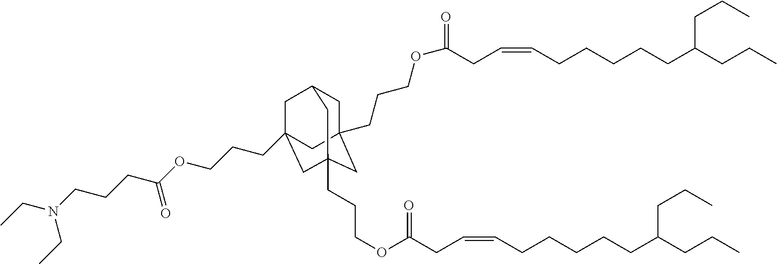 [SMILES: CCCC(CCC)CCCCC/C=C\CC(=O)OCCCC12CC3CC(CCCOC(=O)C/C=C\CCCCCC(CCC)CCC)(C1)CC(CCCOC(=O)CCCN(CC)CC)(C3)C2] |
| AX-152 | 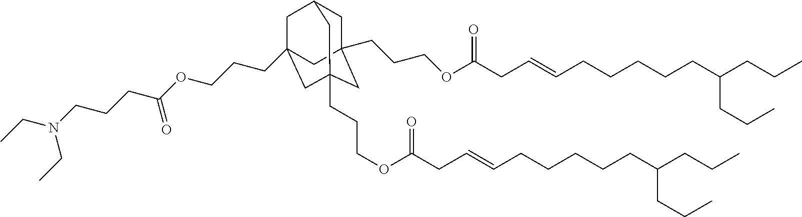 [SMILES: CCCC(CCC)CCCCC/C=C/CC(=O)OCCCC12CC3CC(CCCOC(=O)C/C=C/CCCCCC(CCC)CCC)(C1)CC(CCCOC(=O)CCCN(CC)CC)(C3)C2] |
| AX-153 | 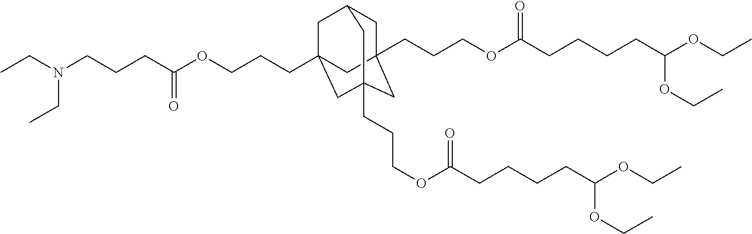 [SMILES: CCOC(CCCCC(=O)OCCCC12CC3CC(CCCOC(=O)CCCCC(OCC)OCC)(C1)CC(CCCOC(=O)CCCN(CC)CC)(C3)C2)OCC] |
| AX-154 | 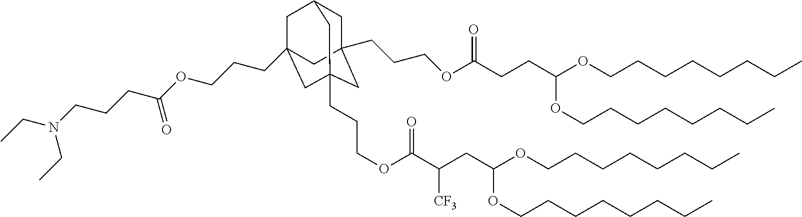 [SMILES: CCCCCCCCOC(CCC(=O)OCCCC12CC3CC(CCCOC(=O)CCCN(CC)CC)(C1)CC(CCCOC(=O)C(CC(OCCCCCCCC)OCCCCCCCC)C(F)(F)F)(C3)C2)OCCCCCCCC] |
| AX-155 | 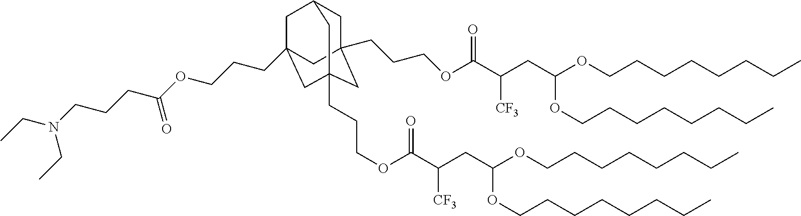 [SMILES: CCCCCCCCOC(CC(C(=O)OCCCC12CC3CC(CCCOC(=O)CCCN(CC)CC)(C1)CC(CCCOC(=O)C(CC(OCCCCCCCC)OCCCCCCCC)C(F)(F)F)(C3)C2)C(F)(F)F)OCCCCCCCC] |
| AX-156 | 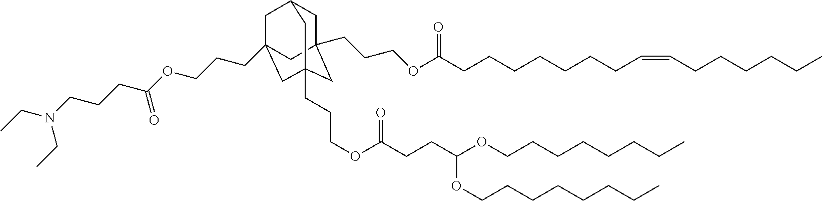 [SMILES: CCCCCC/C=C\CCCCCCCC(=O)OCCCC12CC3CC(CCCOC(=O)CCCN(CC)CC)(C1)CC(CCCOC(=O)CCC(OCCCCCCCC)OCCCCCCCC)(C3)C2] |
| AX-157 | 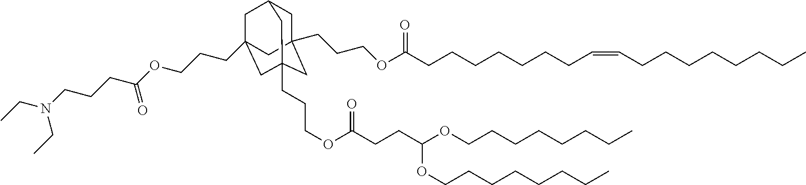 [SMILES: CCCCCCCC/C=C\CCCCCCCC(=O)OCCCC12CC3CC(CCCOC(=O)CCCN(CC)CC)(C1)CC(CCCOC(=O)CCC(OCCCCCCCC)OCCCCCCCC)(C3)C2] |
| AX-158 | 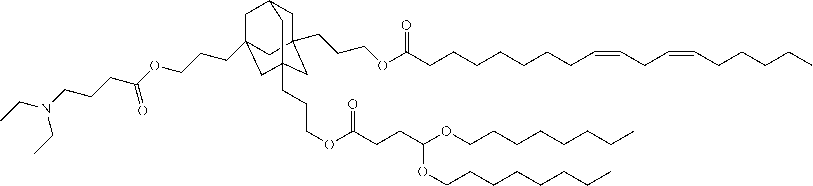 [SMILES: CCCCC/C=C\C/C=C\CCCCCCCC(=O)OCCCC12CC3CC(CCCOC(=O)CCCN(CC)CC)(C1)CC(CCCOC(=O)CCC(OCCCCCCCC)OCCCCCCCC)(C3)C2] |
| AX-159 | 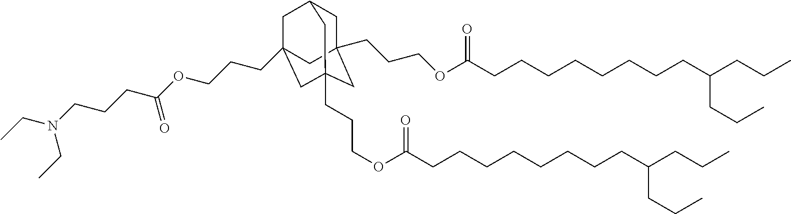 [SMILES: CCCC(CCC)CCCCCCCCC(=O)OCCCC12CC3CC(CCCOC(=O)CCCCCCCCC(CCC)CCC)(C1)CC(CCCOC(=O)CCCN(CC)CC)(C3)C2] |
| AX-160 | 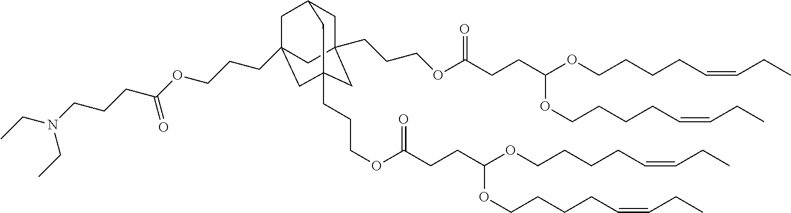 [SMILES: CC/C=C\CCCCOC(CCC(=O)OCCCC12CC3CC(CCCOC(=O)CCCN(CC)CC)(C1)CC(CCCOC(=O)CCC(OCCCC/C=C\CC)OCCCC/C=C\CC)(C3)C2)OCCCC/C=C\CC] |
| AX-161 | 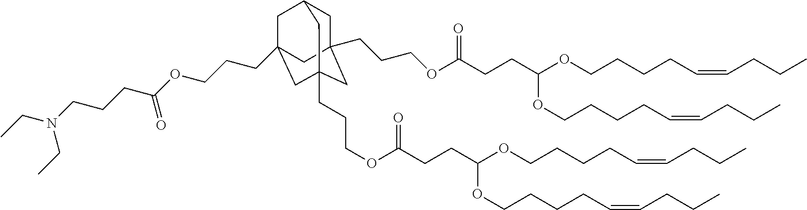 [SMILES: CCC/C=C\CCCCOC(CCC(=O)OCCCC12CC3CC(CCCOC(=O)CCCN(CC)CC)(C1)CC(CCCOC(=O)CCC(OCCCC/C=C\CCC)OCCCC/C=C\CCC)(C3)C2)OCCCC/C=C\CCC] |
| AX-162 | 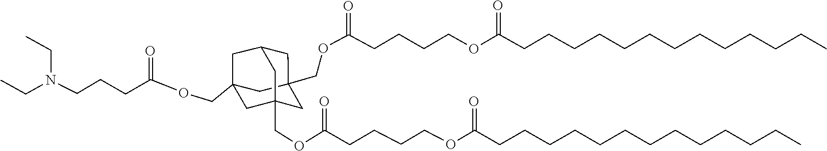 [SMILES: CCCCCCCCCCCCCC(=O)OCCCCC(=O)OCC12CC3CC(COC(=O)CCCCOC(=O)CCCCCCCCCCCCC)(C1)CC(COC(=O)CCCN(CC)CC)(C3)C2] |
| AX-163 | 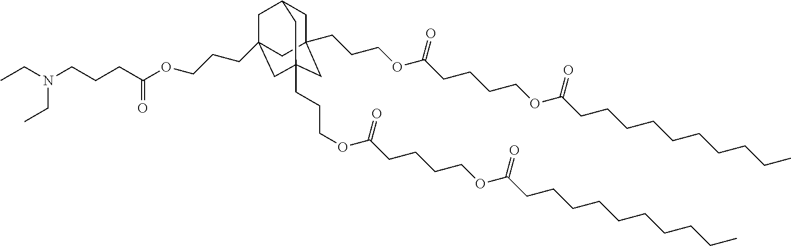 [SMILES: CCCCCCCCCCC(=O)OCCCCC(=O)OCCCC12CC3CC(CCCOC(=O)CCCCOC(=O)CCCCCCCCCC)(C1)CC(CCCOC(=O)CCCN(CC)CC)(C3)C2] |
| AX-164 | 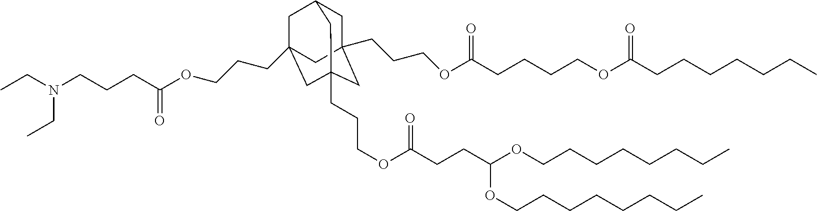 [SMILES: CCCCCCCCOC(CCC(=O)OCCCC12CC3CC(CCCOC(=O)CCCCOC(=O)CCCCCCC)(CC(CCCOC(=O)CCCN(CC)CC)(C3)C1)C2)OCCCCCCCC] |
| AX-165 | 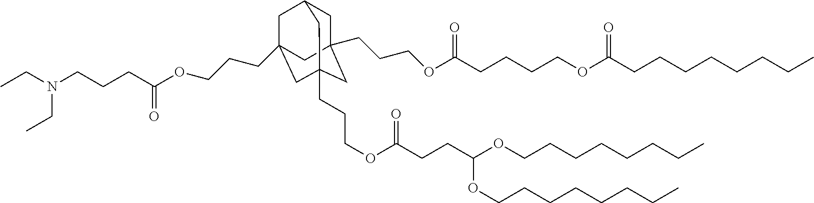 [SMILES: CCCCCCCCOC(CCC(=O)OCCCC12CC3CC(CCCOC(=O)CCCCOC(=O)CCCCCCCC)(CC(CCCOC(=O)CCCN(CC)CC)(C3)C1)C2)OCCCCCCCC] |
| AX-166 | 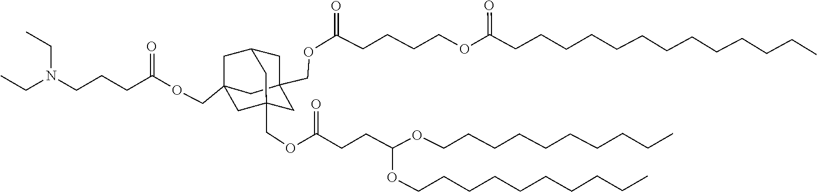 [SMILES: CCCCCCCCCCCCCC(=O)OCCCCC(=O)OCC12CC3CC(COC(=O)CCCN(CC)CC)(C1)CC(COC(=O)CCC(OCCCCCCCCCC)OCCCCCCCCCC)(C3)C2] |
| AX-167 | 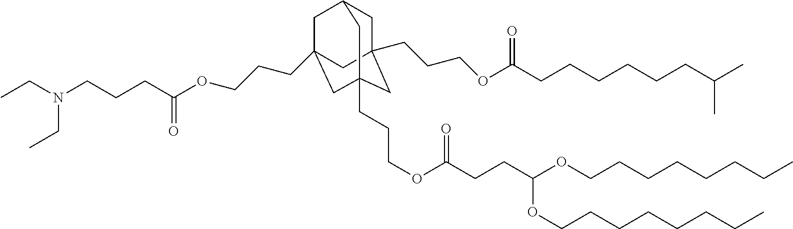 [SMILES: CCCCCCCCOC(CCC(=O)OCCCC12CC3CC(CCCOC(=O)CCCCCCC(C)C)(CC(CCCOC(=O)CCCN(CC)CC)(C3)C1)C2)OCCCCCCCC] |
| AX-168 | 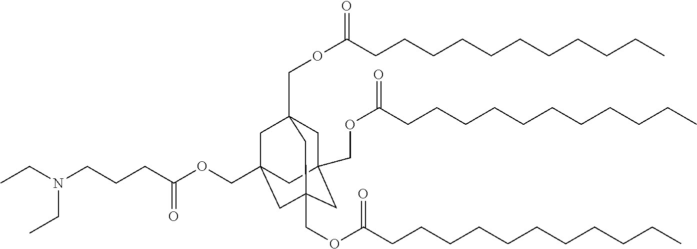 [SMILES: CCCCCCCCCCCC(=O)OCC12CC3(COC(=O)CCCCCCCCCCC)CC(COC(=O)CCCCCCCCCCC)(C1)CC(COC(=O)CCCN(CC)CC)(C2)C3] |
| AX-169 | 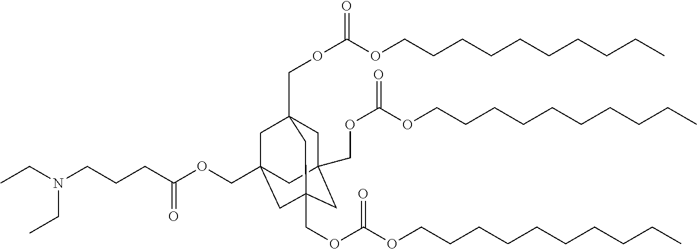 [SMILES: CCCCCCCCCCOC(=O)OCC12CC3(COC(=O)CCCN(CC)CC)CC(COC(=O)OCCCCCCCCCC)(C1)CC(COC(=O)OCCCCCCCCCC)(C3)C2] |
| AX-170 | 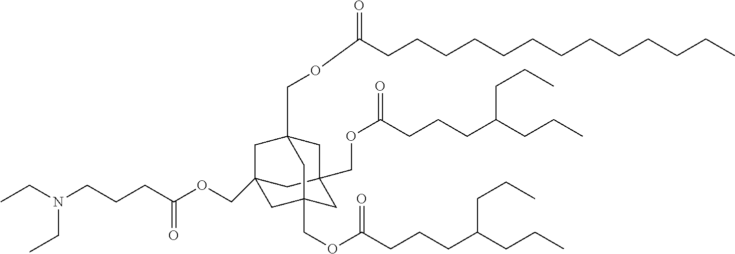 [SMILES: CCCCCCCCCCCCCC(=O)OCC12CC3(COC(=O)CCCC(CCC)CCC)CC(COC(=O)CCCC(CCC)CCC)(C1)CC(COC(=O)CCCN(CC)CC)(C2)C3] |
| AX-171 | 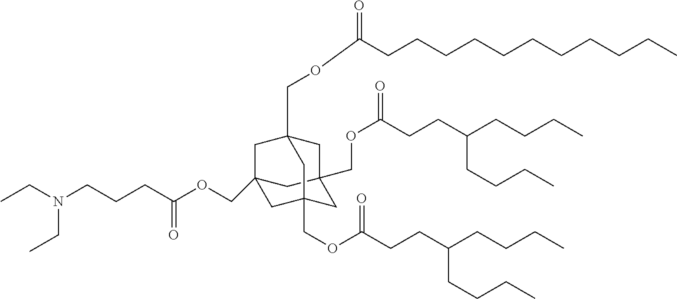 [SMILES: CCCCCCCCCCCC(=O)OCC12CC3(COC(=O)CCCN(CC)CC)CC(COC(=O)CCC(CCCC)CCCC)(C1)CC(COC(=O)CCC(CCCC)CCCC)(C2)C3] |
| AX-172 | 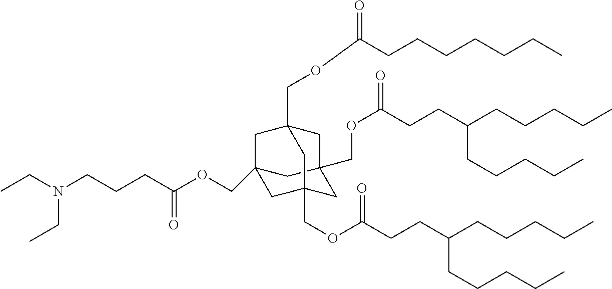 [SMILES: CCCCCCCC(=O)OCC12CC3(COC(=O)CCCN(CC)CC)CC(COC(=O)CCC(CCCCC)CCCCC)(C1)CC(COC(=O)CCC(CCCCC)CCCCC)(C2)C3] |
| AX-173 | 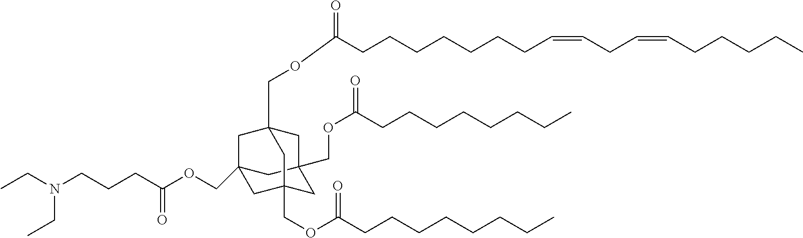 [SMILES: CCCCC/C=C\C/C=C\CCCCCCCC(=O)OCC12CC3(COC(=O)CCCCCCCC)CC(COC(=O)CCCCCCCC)(C1)CC(COC(=O)CCCN(CC)CC)(C3)C2] |
| AX-174 | 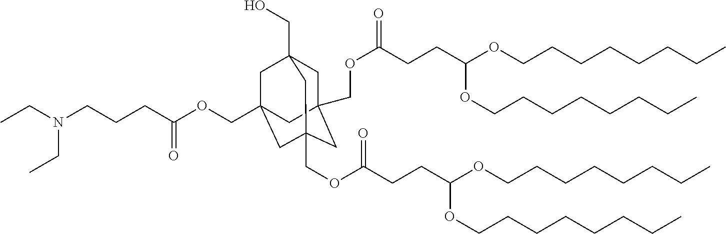 [SMILES: CCCCCCCCOC(CCC(=O)OCC12CC3(CO)CC(COC(=O)CCCN(CC)CC)(C1)CC(COC(=O)CCC(OCCCCCCCC)OCCCCCCCC)(C3)C2)OCCCCCCCC] |
| AX-175 | 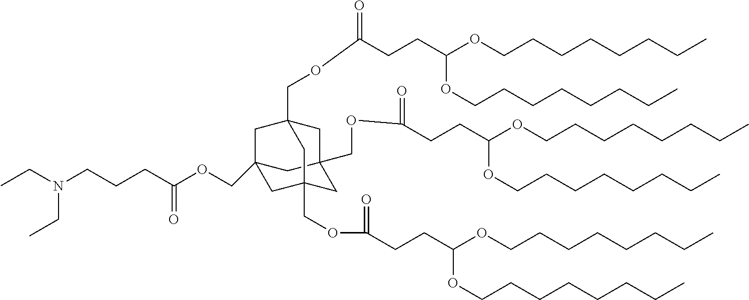 [SMILES: CCCCCCCCOC(CCC(=O)OCC12CC3(COC(=O)CCCN(CC)CC)CC(COC(=O)CCC(OCCCCCCCC)OCCCCCCCC)(C1)CC(COC(=O)CCC(OCCCCCCCC)OCCCCCCCC)(C3)C2)OCCCCCCCC] |
| AX-176 | 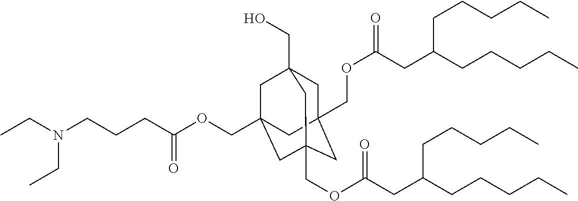 [SMILES: CCCCCC(CCCCC)CC(=O)OCC12CC3(CO)CC(COC(=O)CCCN(CC)CC)(C1)CC(COC(=O)CC(CCCCC)CCCCC)(C3)C2] |
| AX-177 | 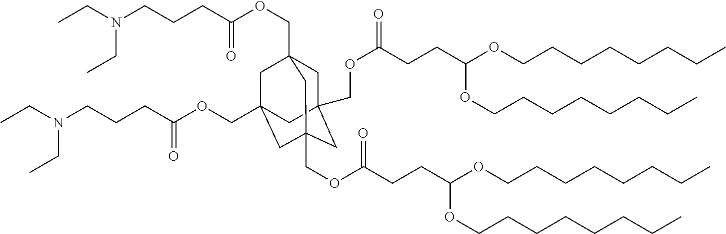 [SMILES: CCCCCCCCOC(CCC(=O)OCC12CC3(COC(=O)CCCN(CC)CC)CC(COC(=O)CCCN(CC)CC)(C1)CC(COC(=O)CCC(OCCCCCCCC)OCCCCCCCC)(C3)C2)OCCCCCCCC] |

### Series “TL”

Described below are a number of exemplary ionizable lipids of the present disclosure.

### Formula (TL)

The present disclosure, in some embodiments, provides compounds of Formula (TL″)

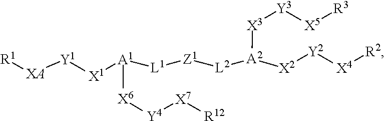

or a pharmaceutically acceptable salt thereof, wherein:
A¹ is selected from CH and N;
A² is selected from CH and N;
L¹ is optionally substituted C₁-C₈ aliphatic, wherein one or more methylene linkages of L¹ are each optionally and independently replaced with —O—, —NH—, —S—, —SS—, —C(O)—, —OC(O)O—, —OC(O)—, —NHC(O)—, or —C(O)O—;
Z¹ is selected from the group consisting of:

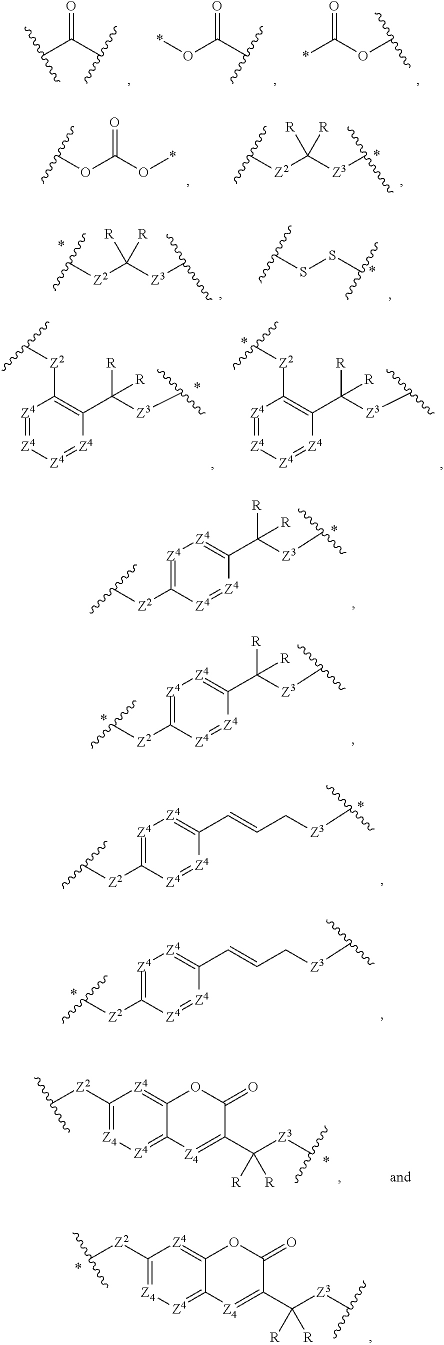

wherein the bond marked with an “*” is attached to L¹
Z² is selected from the group consisting of —O—, —NR—, and —S—;
Z is selected from the group consisting of a bond, —O—, —NR—, —S—, —OC(O)—, —C(O)O—, —NRC(O)—, —C(O)NR—, —NRC(O)O—, or —OC(O)NR—;
each Z⁴ is independently selected from ═CR— and ═N—;
L² is optionally substituted C₁-C₈ aliphatic, wherein one or more metbylene linkages of L are each optionally and independently replaced with —O—, —NH—, —S—, —SS—, —C(O)—, —OC(O)O—, —OC(O)—, —NHC(O)—, or —C(O)O—;
R¹ is selected from the group consisting of —OH, —OAc, —NR₂,

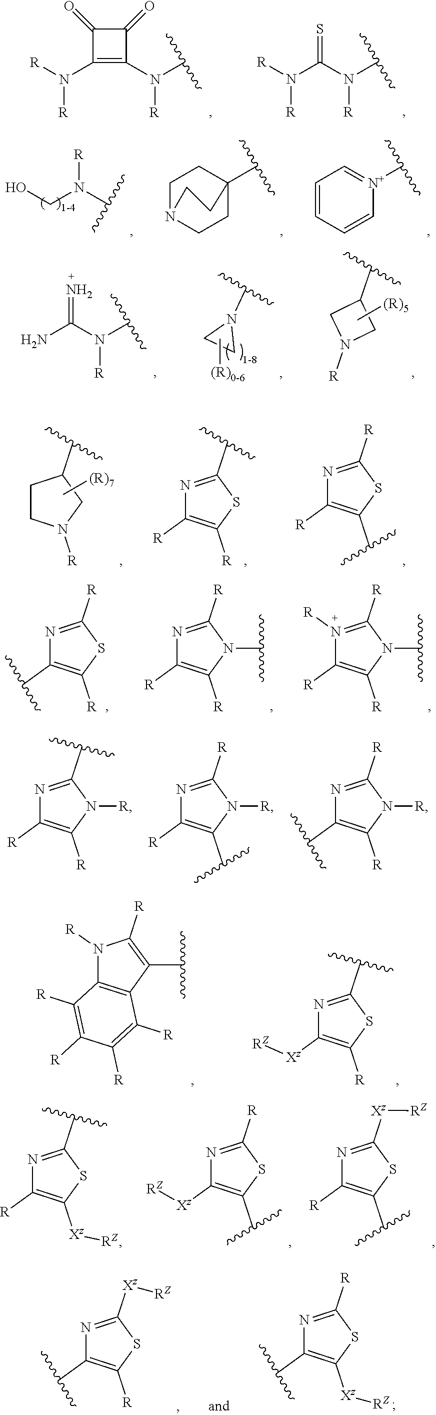

each R is independently —H or C₁-C₆ aliphatic;
X^{Z} is a bond or optionally substituted C₁-C₆ aliphatic;
R^{Z} is NR₂ or OH;
X¹ and X^{A} are each independently a bond or optionally substituted C₁-C₆ aliphatic:
Y¹ is selected from the group consisting of

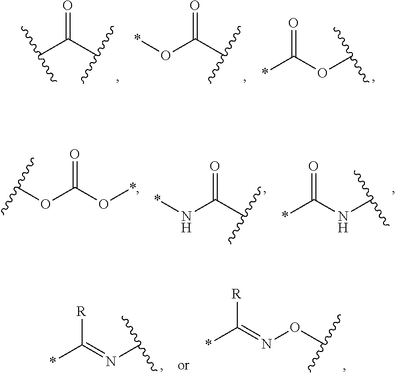

and bond; wherein the bond marked with an “*” is attached to X¹;
each of X², X³, and X⁶ is independently a bond or optionally substituted C₁-C₁₂ aliphatic;
each of Y², Y³, and Y⁴ is independently selected from the group consisting of

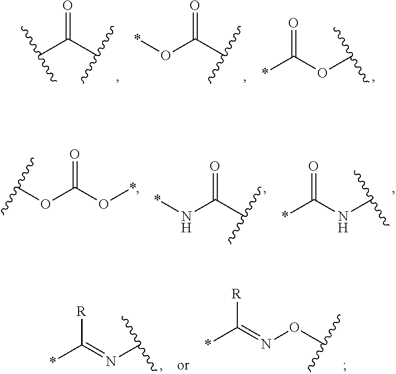

wherein the bond marked with an “*” is attached to X², X³, of X⁶, as appropriate; each of X⁴, X⁵, and X⁷ is independently optionally substituted C₁-C₆ aliphatic;
R² is —CH(OR⁶)(OR⁷), —CH(SR⁶)(SR⁷), —CH(R⁶)(R⁷), —R¹⁰, optionally substituted C₅-C₁₈ aliphatic, or optionally substituted C₁-C₁₄ aliphatic-R¹¹, wherein one or more methylene linkages of R² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R³ is —CH(OR⁸)(OR⁹), —CH(SR⁸)(SR⁹), —CH(R⁸)(R⁹), —R¹¹, optionally substituted C₅-C₁₈ aliphatic, or optionally substituted C₁-C₁₄ aliphatic-R¹¹, wherein one or more methylene linkages of R³ are each optionally and independently replaced with an optionally substituted C₃-C₈ cycloalkylenyl, phenyl, —O—, —NH—. —S—, —SS—, —C(O)—, —OC(O)O—, —OC(O)—, —NHC(O)—, or —C(O)O—; R¹² is —CH(OR¹³)(OR¹⁴), —CH(SR¹³)(SR¹⁴), —CH(R¹³)(R¹⁴), —R¹⁵, optionally substituted C₅-C₁₈ aliphatic, or optionally substituted C₁-C₁₄ aliphatic-R¹⁵, wherein one or more methylene linkages of R¹² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁶ and R⁷ are each independently optionally substituted —C₁-C₁₄ aliphatic, —R¹⁰, or optionally substituted —C₁-C₁₄ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁸ and R⁹ are each independently optionally substituted —C₁-C₁₄ aliphatic, —R¹¹, or optionally substituted —C₁-C₁₄ aliphatic-R¹¹; wherein one or more methylene linkages of R⁸ and R⁹ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R¹³ and R¹⁴ are each independently optionally substituted —C₁-C₁₄ aliphatic, —R¹⁵, or optionally substituted —C₁-C₁₄ aliphatic-R¹⁵; wherein one or more methylene linkages of R¹³ and R¹⁴ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each of R¹⁰, R¹¹, and R¹⁵ is independently an optionally substituted cylic, bicyclic, bridged bicyclic, multicyclic or bridged multicyclic C₄-C₁₄ cycloalkyl or optionally substituted cylic, bicyclic, bridged bicyclic, multicyclic or bridged multicyclic 4-14 membered heterocyclyl, or two R¹⁰, R¹¹ or R¹⁵ are taken together form an optionally substituted bridged bicyclic or multicyclic C₄-C₁₄ cycloalkyl or optionally substituted bridged bicyclic or multicyclic 4-14 membered heterocyclyl.

### Additional Formulae

The present disclosure, in some embodiments, provides compounds of any one of the Formulae below:

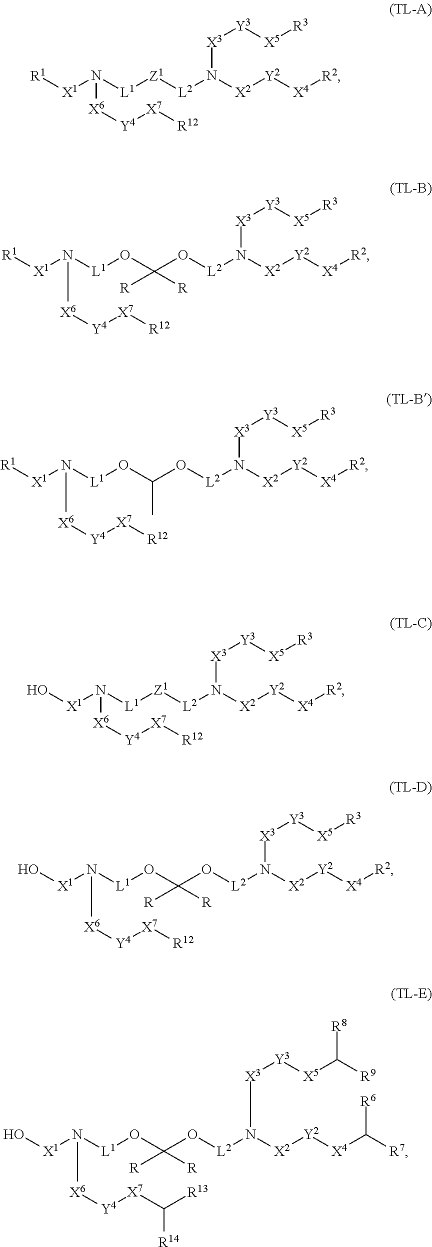

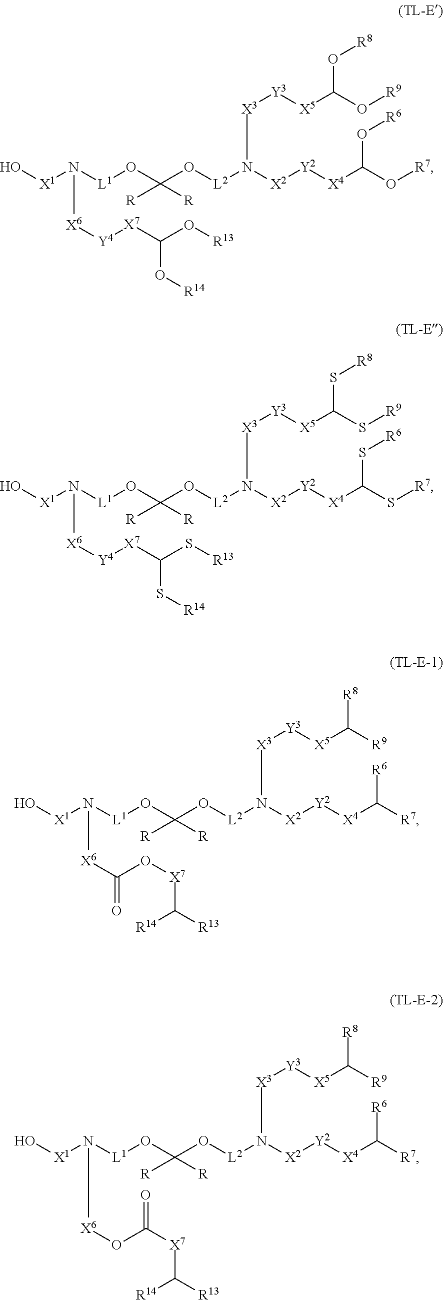

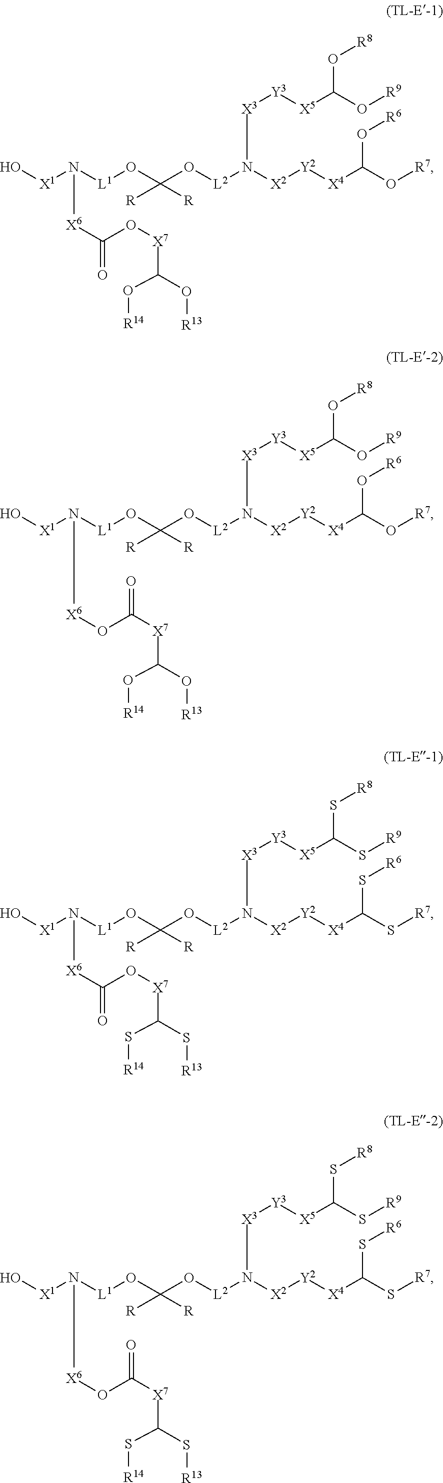

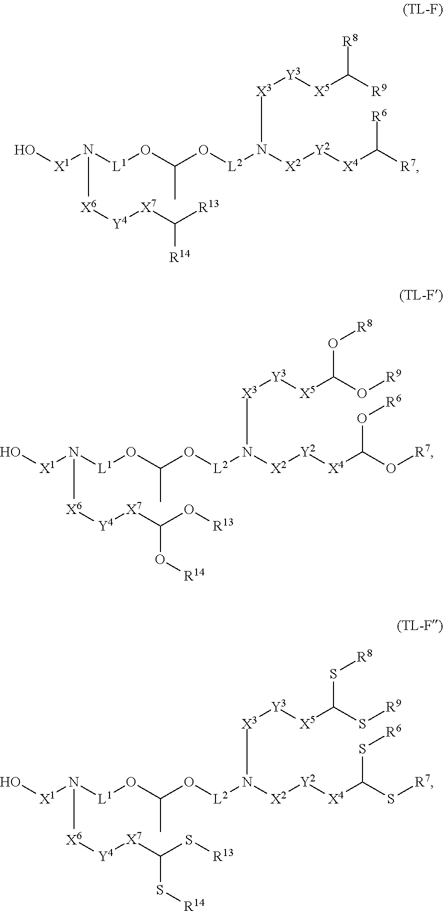

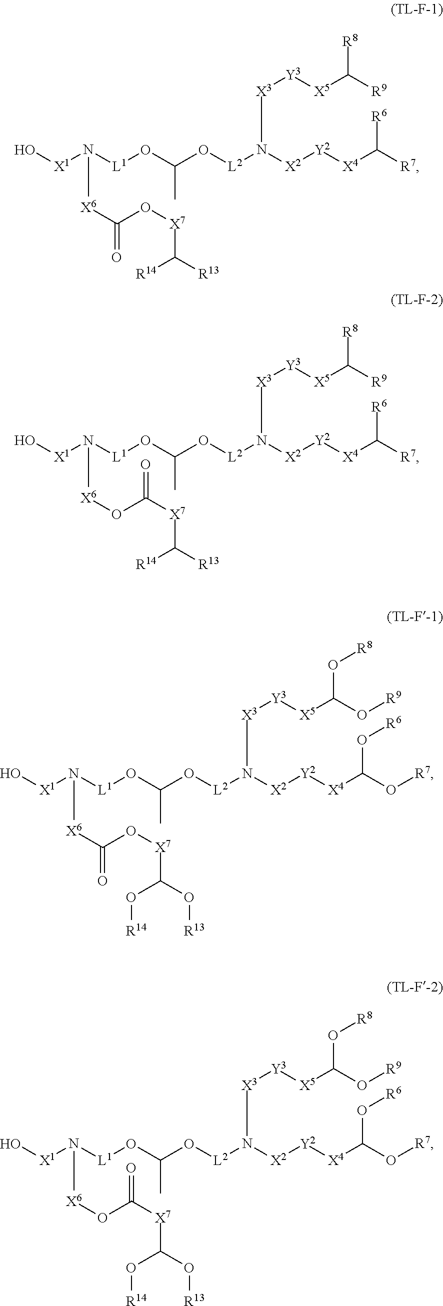

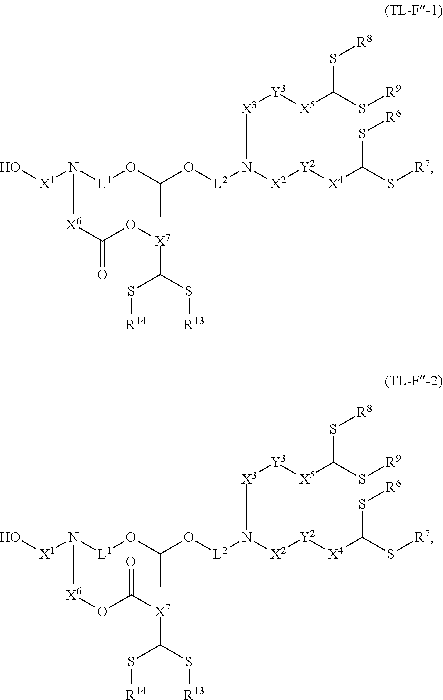

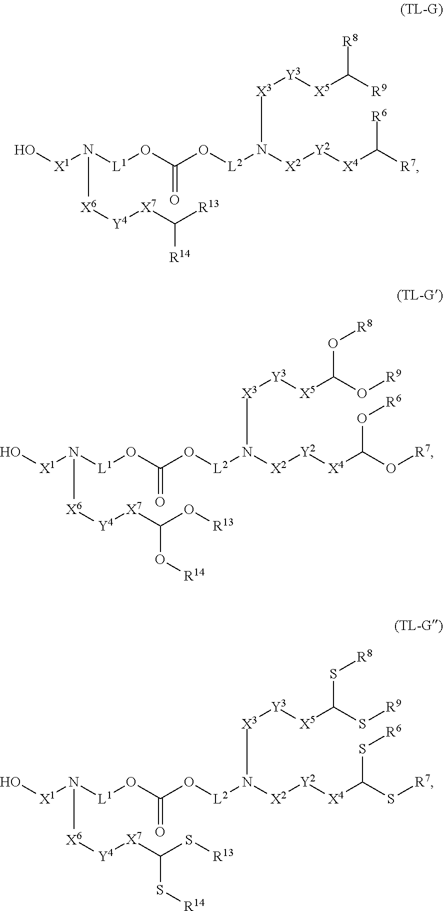

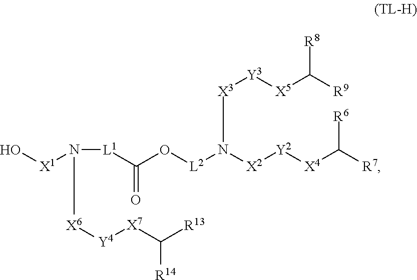

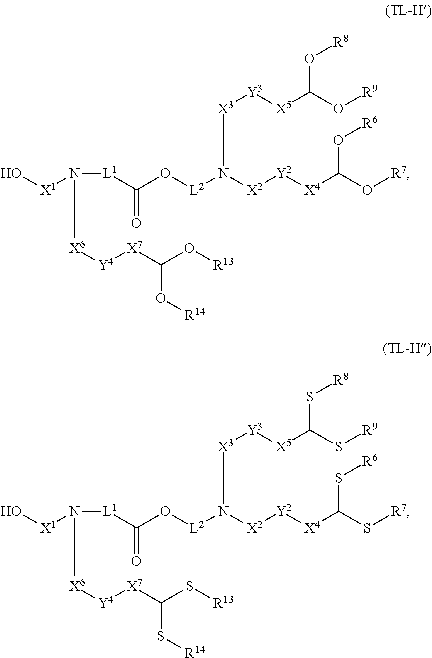

Wherein R¹, R, X¹, L¹, L², Z¹, X², X³, X⁴, X⁵, Y², Y³, Y⁴, R², R³, R⁶, R⁷, R⁸, R⁹, R¹⁰, R¹¹, R¹², R¹³, R¹⁴, and R¹⁵ are as described in Formula (TL″).

In some embodiments, ionizable lipids of the present disclosure are selected from any lipid in Table (I-K) below or a pharmaceutically acceptable salt, solvate, stereoisomer, or enantiomer thereof:

TABLE (I-K)
Non-Limiting Examples of Ionizable lipids of the present disclosure

| Cmpd | Structure |
|---|---|
| TL-1 | 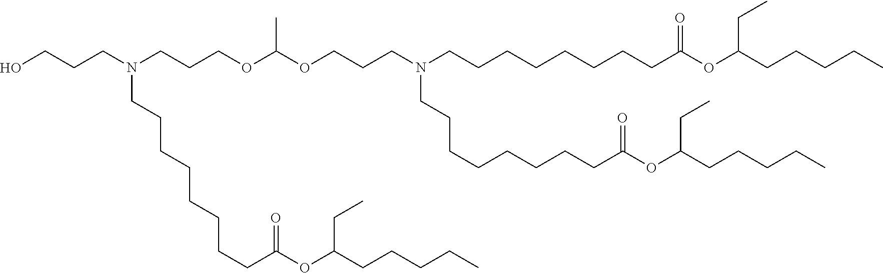 [SMILES: CCCCCC(CC)OC(=O)CCCCCCCCN(CCCO)CCCOC(C)OCCCN(CCCCCCCCC(=O)OC(CC)CCCCC)CCCCCCCCC(=O)OC(CC)CCCCC] |
| TL-2 | 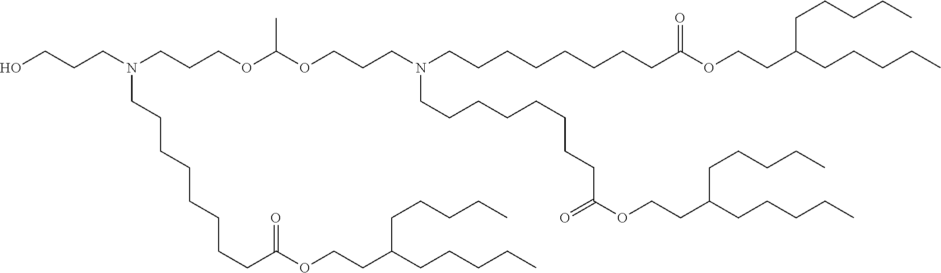 [SMILES: CCCCCC(CCCCC)CCOC(=O)CCCCCCCCN(CCCO)CCCOC(C)OCCCN(CCCCCCCCC(=O)OCCC(CCCCC)CCCCC)CCCCCCCCC(=O)OCCC(CCCCC)CCCCC] |
| TL-3 | 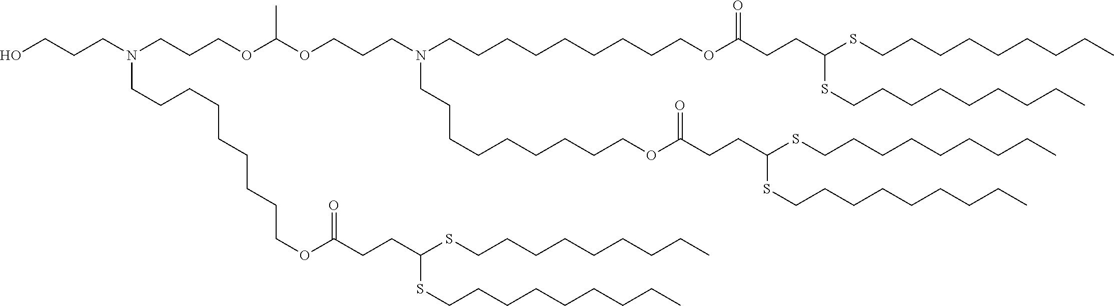 [SMILES: CCCCCCCCCSC(CCC(=O)OCCCCCCCCCN(CCCO)CCCOC(C)OCCCN(CCCCCCCCCOC(=O)CCC(SCCCCCCCCC)SCCCCCCCCC)CCCCCCCCCOC(=O)CCC(SCCCCCCCCC)SCCCCCCCCC)SCCCCCCCCC] |
| TL-4 | 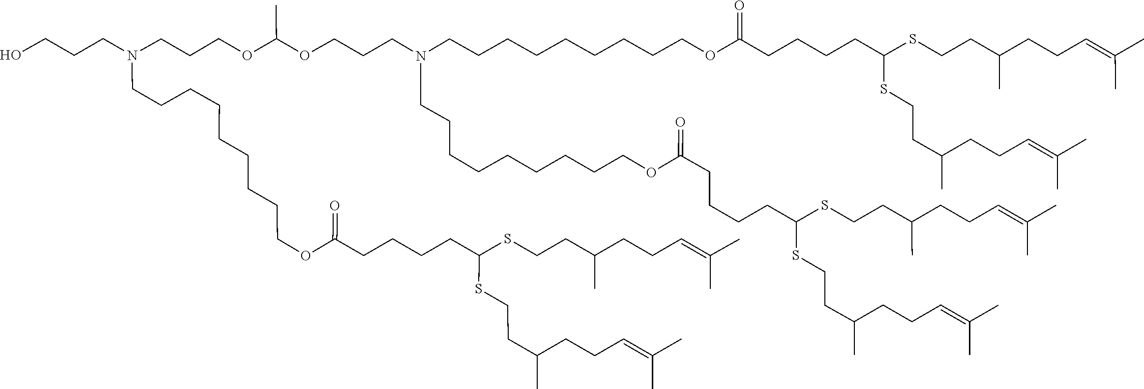 [SMILES: CC(C)=CCCC(C)CCSC(CCCCC(=O)OCCCCCCCCCN(CCCO)CCCOC(C)OCCCN(CCCCCCCCCOC(=O)CCCCC(SCCC(C)CCC=C(C)C)SCCC(C)CCC=C(C)C)CCCCCCCCCOC(=O)CCCCC(SCCC(C)CCC=C(C)C)SCCC(C)CCC=C(C)C)SCCC(C)CCC=C(C)C] |
| TL-5 | 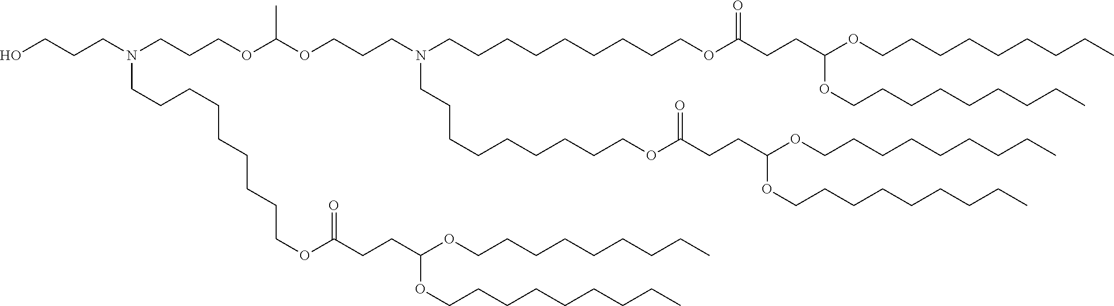 [SMILES: CCCCCCCCCOC(CCC(=O)OCCCCCCCCCN(CCCO)CCCOC(C)OCCCN(CCCCCCCCCOC(=O)CCC(OCCCCCCCCC)OCCCCCCCCC)CCCCCCCCCOC(=O)CCC(OCCCCCCCCC)OCCCCCCCCC)OCCCCCCCCC] |
| TL-6 | 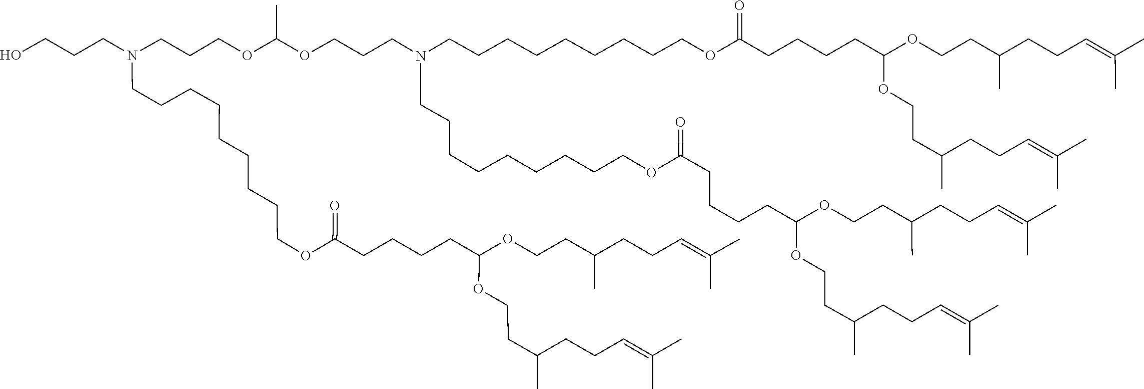 [SMILES: CC(C)=CCCC(C)CCOC(CCCCC(=O)OCCCCCCCCCN(CCCO)CCCOC(C)OCCCN(CCCCCCCCCOC(=O)CCCCC(OCCC(C)CCC=C(C)C)OCCC(C)CCC=C(C)C)CCCCCCCCCOC(=O)CCCCC(OCCC(C)CCC=C(C)C)OCCC(C)CCC=C(C)C)OCCC(C)CCC=C(C)C] |
| TL-7 | 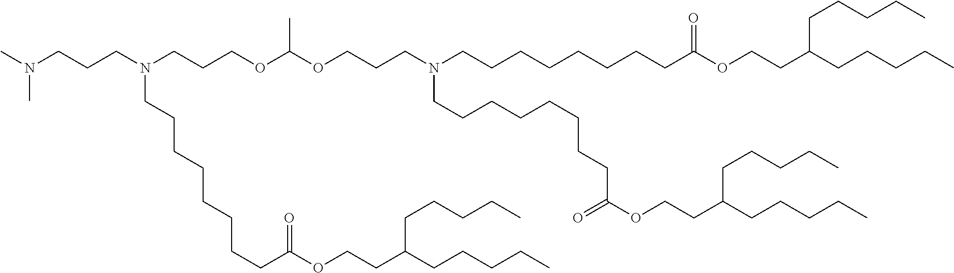 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCCN(C)C] |
| TL-8 | 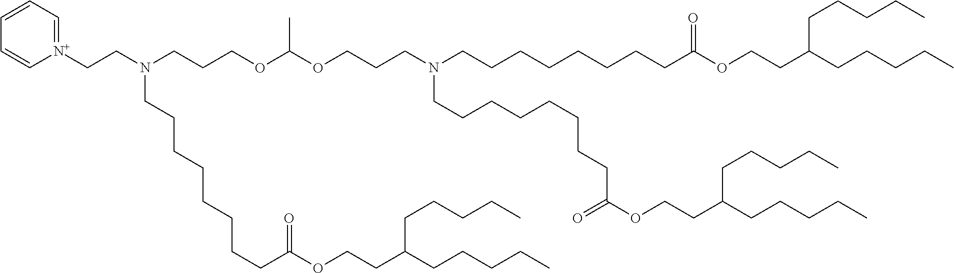 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CC[n+]1ccccc1] |
| TL-9 | 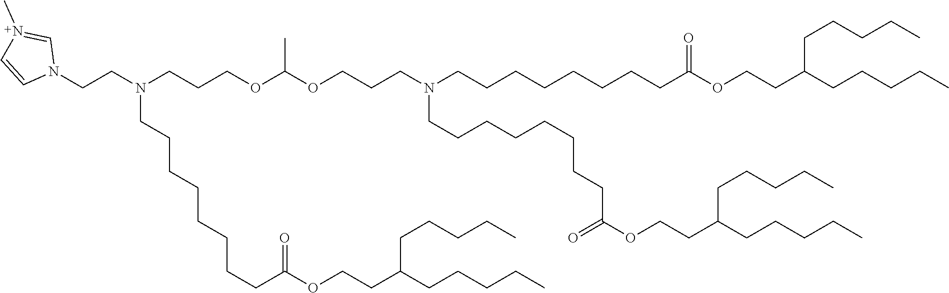 |
| TL-10 | 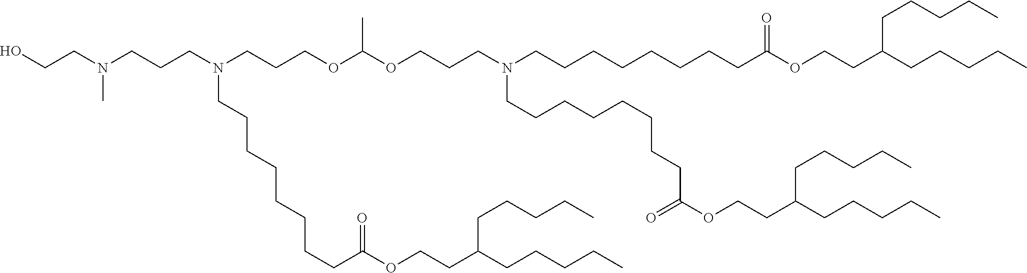 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCCN(C)CCO] |
| TL-11 | 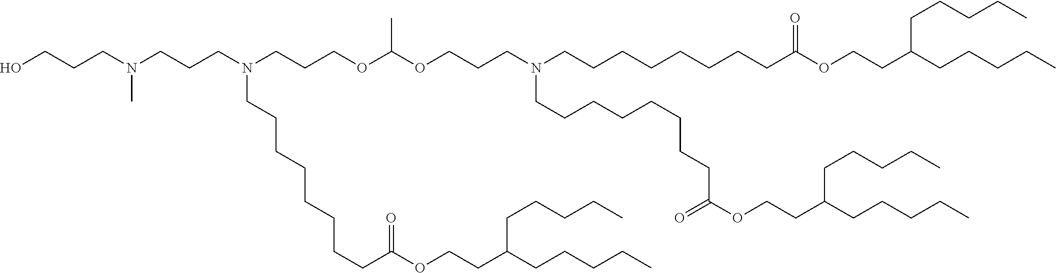 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCCN(C)CCCO] |
| TL-12 | 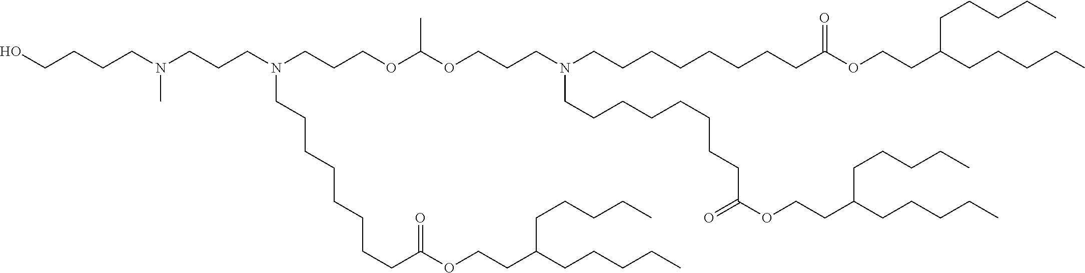 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCCN(C)CCCCO] |
| TL-13 | 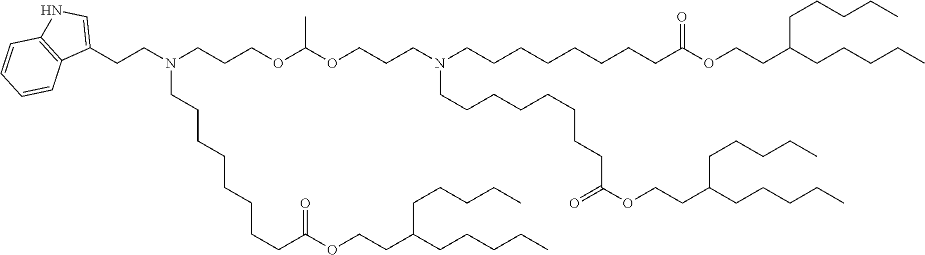 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCc1c[nH]c2ccccc12] |
| TL-14 | 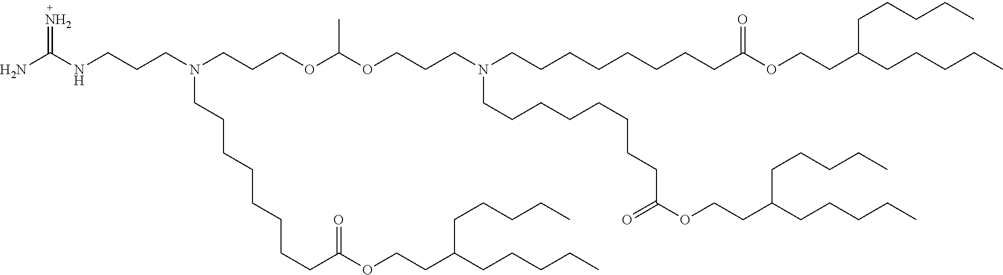 [SMILES: CCCCCC(CCCCC)CCOC(=O)CCCCCCCCN(CCCCCCCCC(=O)OCCC(CCCCC)CCCCC)CCCOC(C)OCCCN(CCCCCCCCC(=O)OCCC(CCCCC)CCCCC)CCCNC(=N)N] |
| TL-15 | 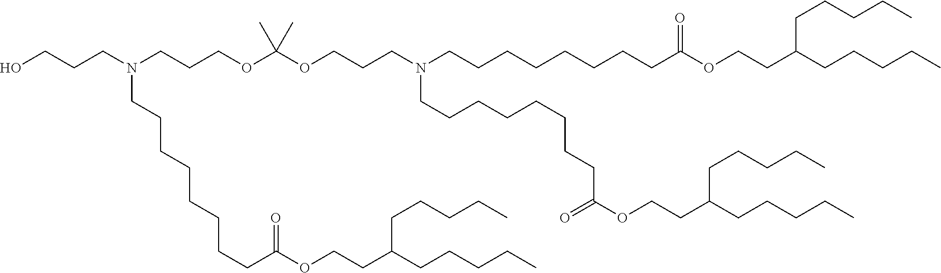 [SMILES: CCCCCC(CCCCC)CCOC(=O)CCCCCCCCN(CCCO)CCCOC(C)(C)OCCCN(CCCCCCCCC(=O)OCCC(CCCCC)CCCCC)CCCCCCCCC(=O)OCCC(CCCCC)CCCCC] |
| TL-16 | 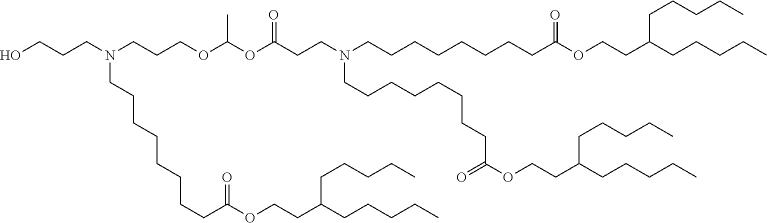 [SMILES: CCCCCC(CCCCC)CCOC(=O)CCCCCCCCN(CCCO)CCCOC(C)OC(=O)CCN(CCCCCCCCC(=O)OCCC(CCCCC)CCCCC)CCCCCCCCC(=O)OCCC(CCCCC)CCCCC] |
| TL-17 | 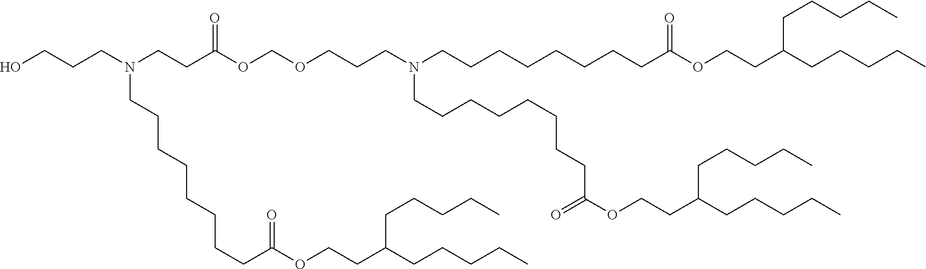 [SMILES: CCCCCC(CCCCC)CCOC(=O)CCCCCCCCN(CCCCCCCCC(=O)OCCC(CCCCC)CCCCC)CCCOCOC(=O)CCN(CCCO)CCCCCCCCC(=O)OCCC(CCCCC)CCCCC] |
| TL-18 | 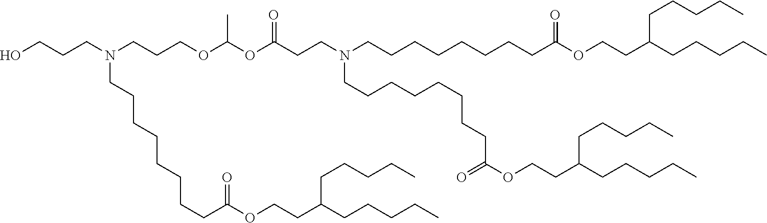 [SMILES: CCCCCC(CCCCC)CCOC(=O)CCCCCCCCN(CCCO)CCCOC(C)OC(=O)CCN(CCCCCCCCC(=O)OCCC(CCCCC)CCCCC)CCCCCCCCC(=O)OCCC(CCCCC)CCCCC] |
| TL-19 | 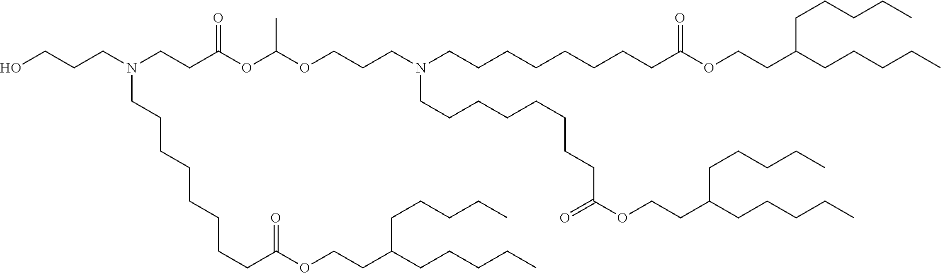 [SMILES: CCCCCC(CCCCC)CCOC(=O)CCCCCCCCN(CCCCCCCCC(=O)OCCC(CCCCC)CCCCC)CCCOC(C)OC(=O)CCN(CCCO)CCCCCCCCC(=O)OCCC(CCCCC)CCCCC] |
| TL-20 | 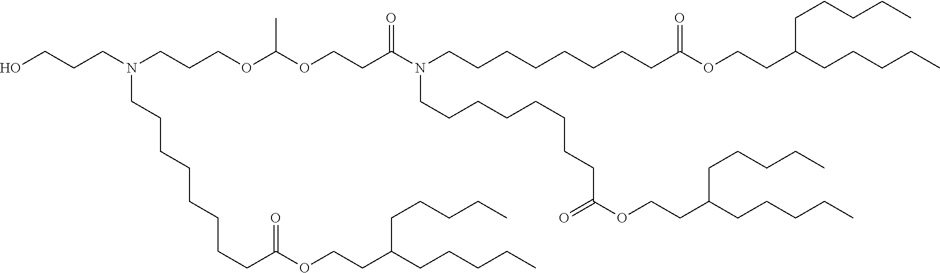 [SMILES: CCCCCC(CCCCC)CCOC(=O)CCCCCCCCN(CCCO)CCCOC(C)OCCC(=O)N(CCCCCCCCC(=O)OCCC(CCCCC)CCCCC)CCCCCCCCC(=O)OCCC(CCCCC)CCCCC] |
| TL-21 | 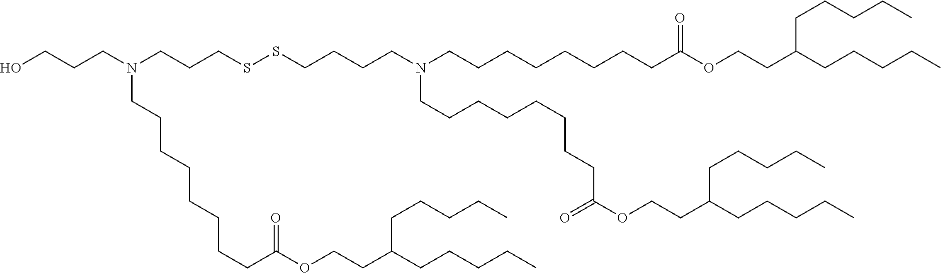 [SMILES: CCCCCC(CCCCC)CCOC(=O)CCCCCCCCN(CCCCCCCCC(=O)OCCC(CCCCC)CCCCC)CCCCSSCCCN(CCCO)CCCCCCCCC(=O)OCCC(CCCCC)CCCCC] |
| TL-22 | 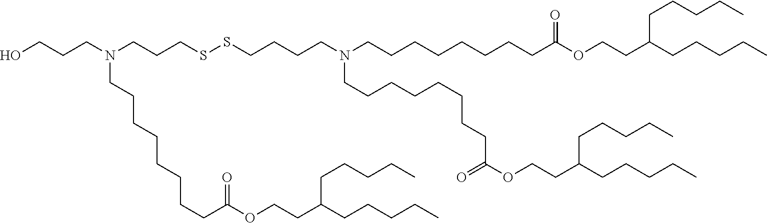 [SMILES: CCCCCC(CCCCC)CCOC(=O)CCCCCCCCN(CCCCCCCCC(=O)OCCC(CCCCC)CCCCC)CCCCSSCCCN(CCCO)CCCCCCCCC(=O)OCCC(CCCCC)CCCCC] |
| TL-23 | 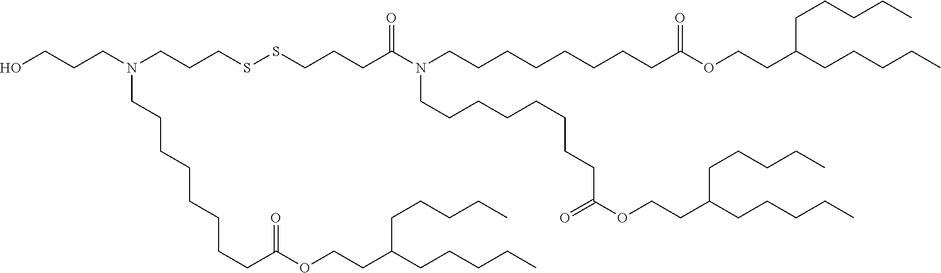 [SMILES: CCCCCC(CCCCC)CCOC(=O)CCCCCCCCN(CCCO)CCCSSCCCC(=O)N(CCCCCCCCC(=O)OCCC(CCCCC)CCCCC)CCCCCCCCC(=O)OCCC(CCCCC)CCCCC] |
| TL-24 | 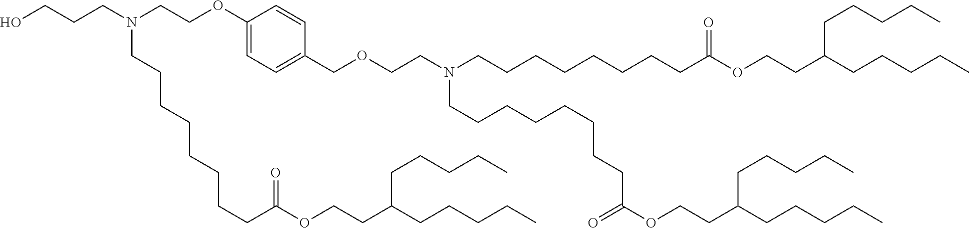 [SMILES: CCCCCC(CCCCC)CCOC(=O)CCCCCCCCN(CCCCCCCCC(=O)OCCC(CCCCC)CCCCC)CCOCc1ccc(OCCN(CCCO)CCCCCCCCC(=O)OCCC(CCCCC)CCCCC)cc1] |
| TL-25 | 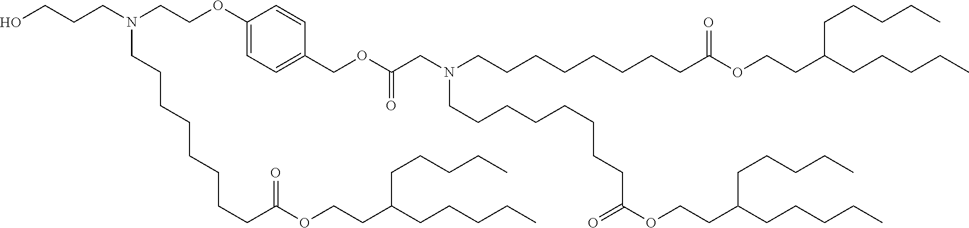 [SMILES: CCCCCC(CCCCC)CCOC(=O)CCCCCCCCN(CCCO)CCOc1ccc(COC(=O)CN(CCCCCCCCC(=O)OCCC(CCCCC)CCCCC)CCCCCCCCC(=O)OCCC(CCCCC)CCCCC)cc1] |
| TL-26 | 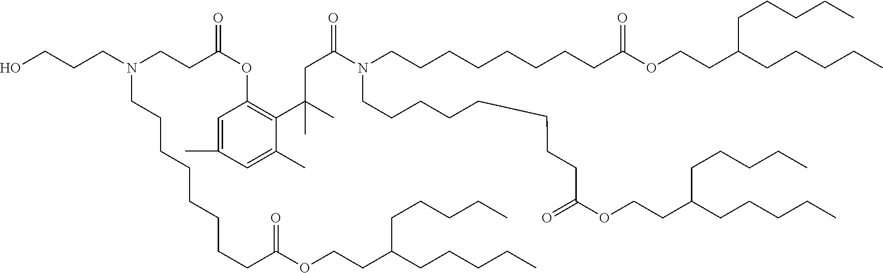 [SMILES: CCCCCC(CCCCC)CCOC(=O)CCCCCCCCN(CCCO)CCC(=O)Oc1cc(C)cc(C)c1C(C)(C)CC(=O)N(CCCCCCCCC(=O)OCCC(CCCCC)CCCCC)CCCCCCCCC(=O)OCCC(CCCCC)CCCCC] |
| TL-27 | 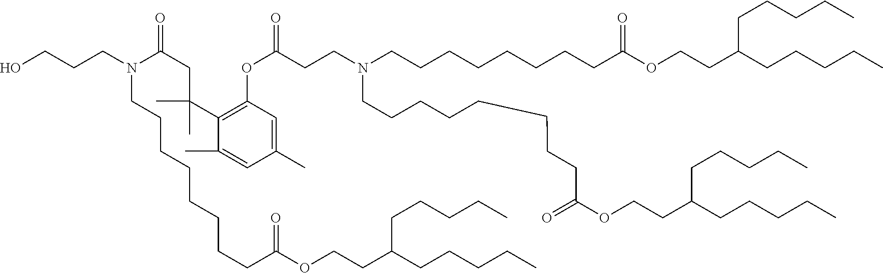 [SMILES: CCCCCC(CCCCC)CCOC(=O)CCCCCCCCN(CCCCCCCCC(=O)OCCC(CCCCC)CCCCC)CCC(=O)Oc1cc(C)cc(C)c1C(C)(C)CC(=O)N(CCCO)CCCCCCCCC(=O)OCCC(CCCCC)CCCCC] |
| TL-28 | 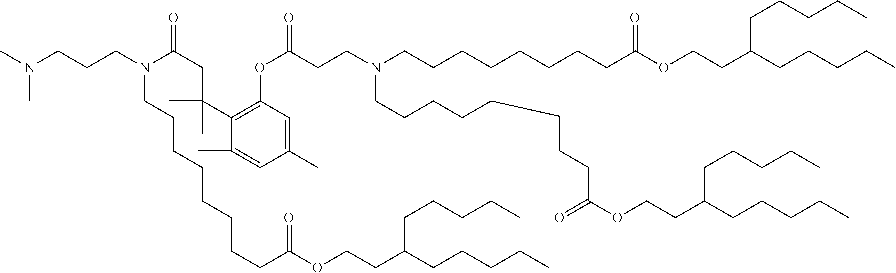 [SMILES: CCCCCC(CCCCC)CCOC(=O)CCCCCCCCN(CCCCCCCCC(=O)OCCC(CCCCC)CCCCC)CCC(=O)Oc1cc(C)cc(C)c1C(C)(C)CC(=O)N(CCCCCCCCC(=O)OCCC(CCCCC)CCCCC)CCCN(C)C] |
| TL-29 | 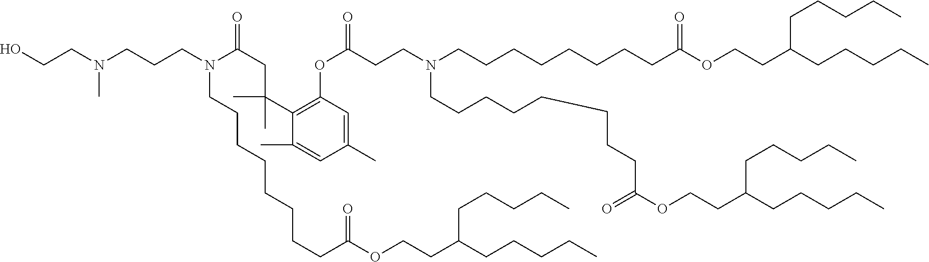 [SMILES: CCCCCC(CCCCC)CCOC(=O)CCCCCCCCN(CCCCCCCCC(=O)OCCC(CCCCC)CCCCC)CCC(=O)Oc1cc(C)cc(C)c1C(C)(C)CC(=O)N(CCCCCCCCC(=O)OCCC(CCCCC)CCCCC)CCCN(C)CCO] |
| TL-30 | 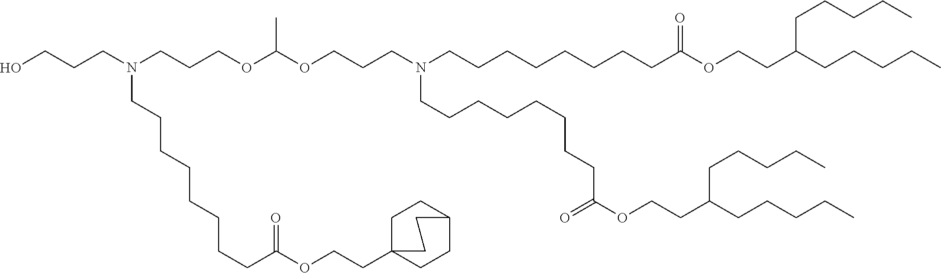 [SMILES: CCCCCC(CCCCC)CCOC(=O)CCCCCCCCN(CCCCCCCCC(=O)OCCC(CCCCC)CCCCC)CCCOC(C)OCCCN(CCCO)CCCCCCCCC(=O)OCCC12CCC(CC1)CC2] |
| TL-31 | 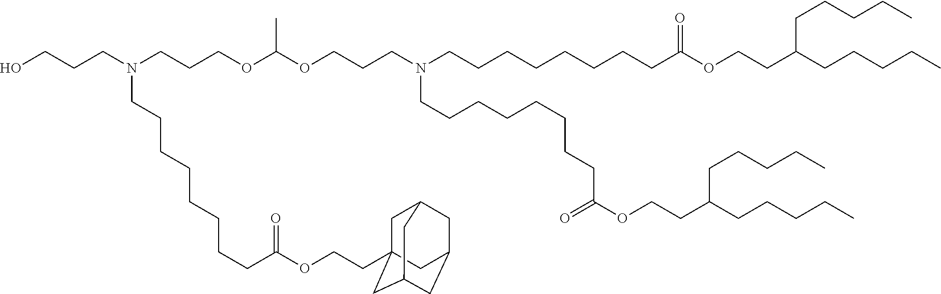 [SMILES: CCCCCC(CCCCC)CCOC(=O)CCCCCCCCN(CCCCCCCCC(=O)OCCC(CCCCC)CCCCC)CCCOC(C)OCCCN(CCCO)CCCCCCCCC(=O)OCCC12CC3CC(CC(C3)C1)C2] |
| TL-32 | 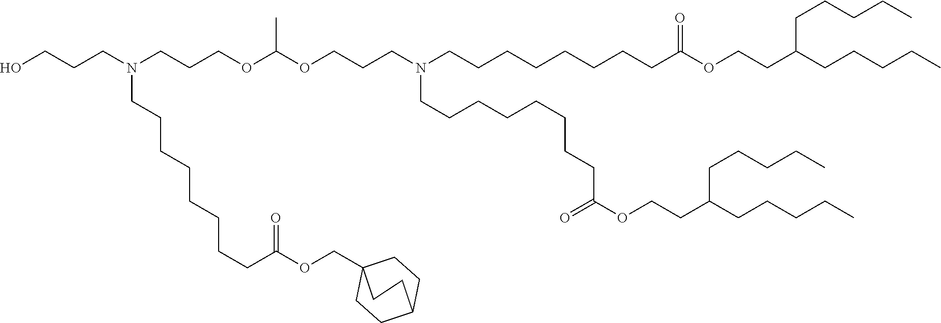 [SMILES: CCCCCC(CCCCC)CCOC(=O)CCCCCCCCN(CCCCCCCCC(=O)OCCC(CCCCC)CCCCC)CCCOC(C)OCCCN(CCCO)CCCCCCCCC(=O)OCC12CCC(CC1)CC2] |
| TL-33 | 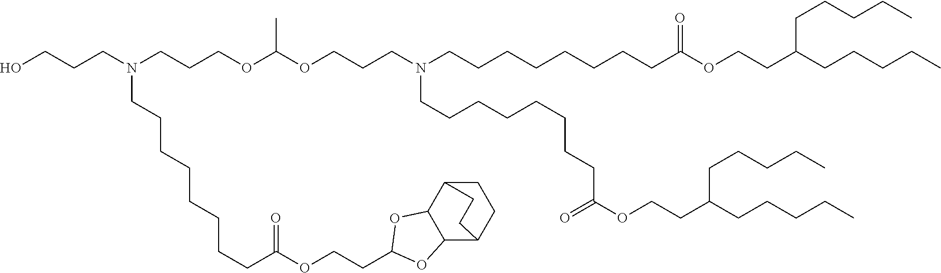 [SMILES: CCCCCC(CCCCC)CCOC(=O)CCCCCCCCN(CCCCCCCCC(=O)OCCC(CCCCC)CCCCC)CCCOC(C)OCCCN(CCCO)CCCCCCCCC(=O)OCCC1OC2C3CCC(CC3)C2O1] |
| TL-34 | 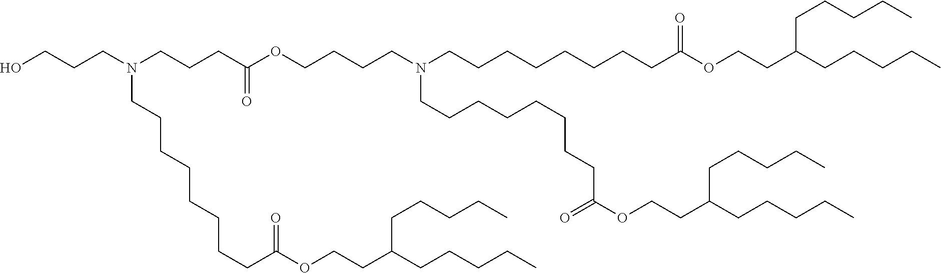 [SMILES: CCCCCC(CCCCC)CCOC(=O)CCCCCCCCN(CCCCCCCCC(=O)OCCC(CCCCC)CCCCC)CCCCOC(=O)CCCN(CCCO)CCCCCCCCC(=O)OCCC(CCCCC)CCCCC] |
| TL-35 | 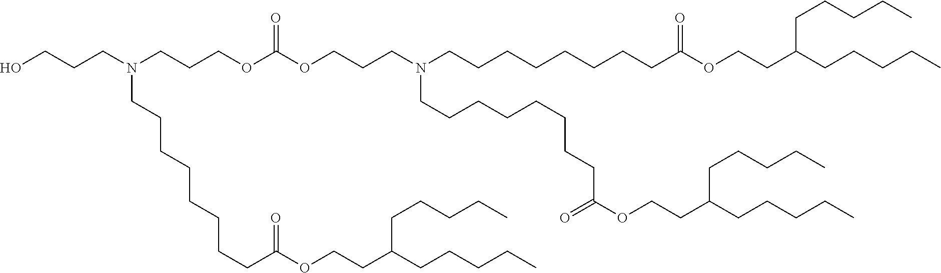 [SMILES: CCCCCC(CCCCC)CCOC(=O)CCCCCCCCN(CCCO)CCCOC(=O)OCCCN(CCCCCCCCC(=O)OCCC(CCCCC)CCCCC)CCCCCCCCC(=O)OCCC(CCCCC)CCCCC] |
| TL-36 | 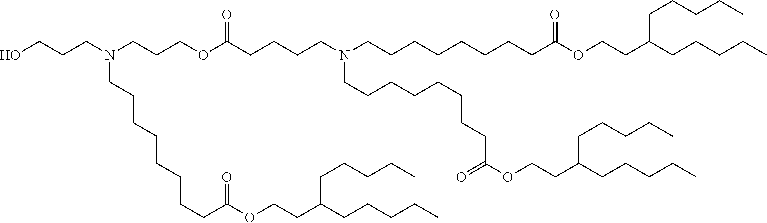 [SMILES: CCCCCC(CCCCC)CCOC(=O)CCCCCCCCN(CCCCCCCCC(=O)OCCC(CCCCC)CCCCC)CCCCC(=O)OCCCN(CCCO)CCCCCCCCC(=O)OCCC(CCCCC)CCCCC] |
| TL-37 | 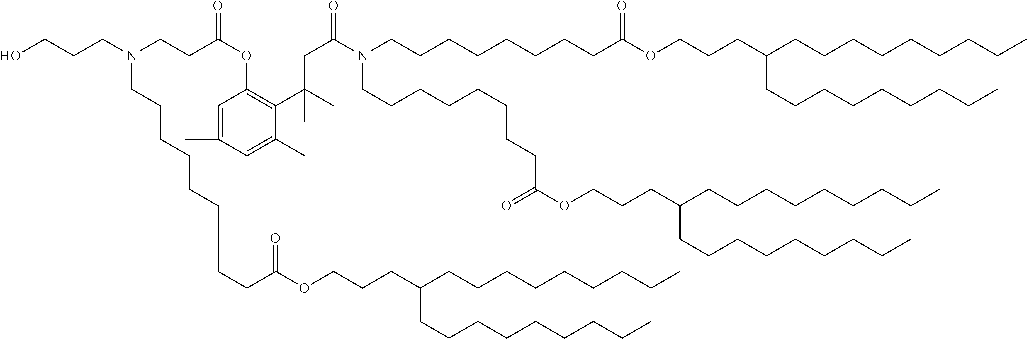 [SMILES: CCCCCCCCCC(CCCCCCCCC)CCCOC(=O)CCCCCCCCN(CCCO)CCC(=O)Oc1cc(C)cc(C)c1C(C)(C)CC(=O)N(CCCCCCCCC(=O)OCCCC(CCCCCCCCC)CCCCCCCCC)CCCCCCCCC(=O)OCCCC(CCCCCCCCC)CCCCCCCCC] |
| TL-38 | 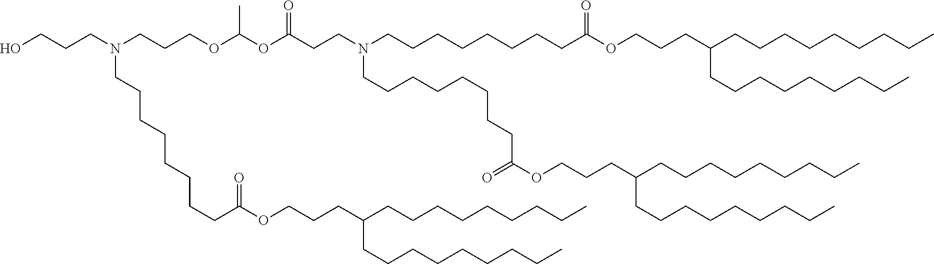 [SMILES: CCCCCCCCCC(CCCCCCCCC)CCCOC(=O)CCCCCCCCN(CCCO)CCCOC(C)OC(=O)CCN(CCCCCCCCC(=O)OCCCC(CCCCCCCCC)CCCCCCCCC)CCCCCCCCC(=O)OCCCC(CCCCCCCCC)CCCCCCCCC] |
| TL-39 | 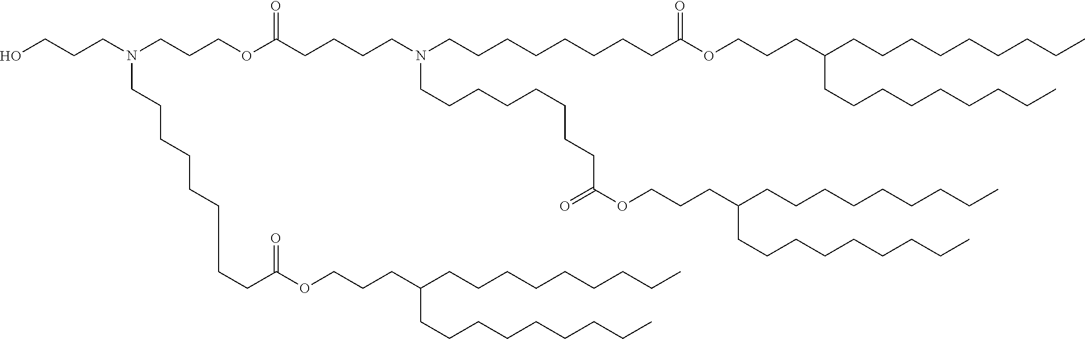 [SMILES: CCCCCCCCCC(CCCCCCCCC)CCCOC(=O)CCCCCCCCN(CCCCCCCCC(=O)OCCCC(CCCCCCCCC)CCCCCCCCC)CCCCC(=O)OCCCN(CCCO)CCCCCCCCC(=O)OCCCC(CCCCCCCCC)CCCCCCCCC] |
| TL-40 | 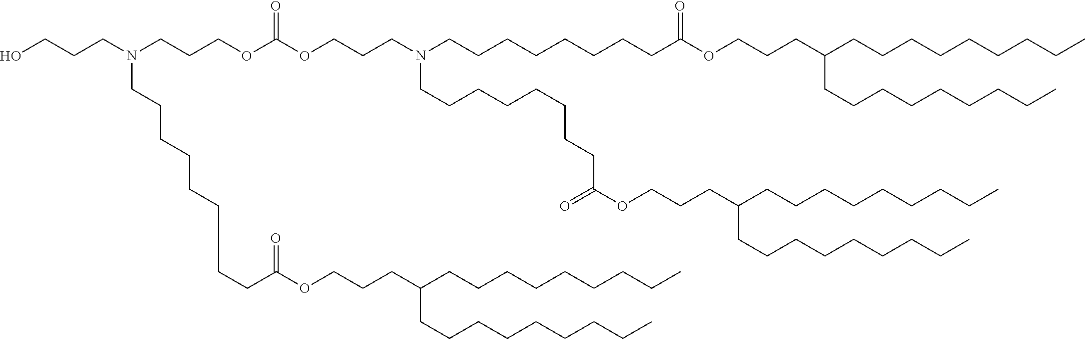 [SMILES: CCCCCCCCCC(CCCCCCCCC)CCCOC(=O)CCCCCCCCN(CCCO)CCCOC(=O)OCCCN(CCCCCCCCC(=O)OCCCC(CCCCCCCCC)CCCCCCCCC)CCCCCCCCC(=O)OCCCC(CCCCCCCCC)CCCCCCCCC] |
| TL-41 | 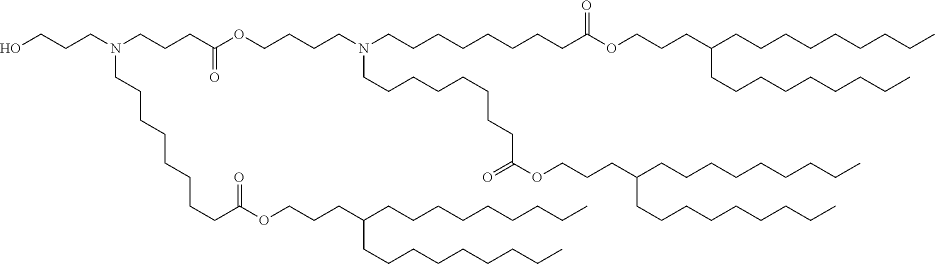 [SMILES: CCCCCCCCCC(CCCCCCCCC)CCCOC(=O)CCCCCCCCN(CCCCCCCCC(=O)OCCCC(CCCCCCCCC)CCCCCCCCC)CCCCOC(=O)CCCN(CCCO)CCCCCCCCC(=O)OCCCC(CCCCCCCCC)CCCCCCCCC] |
| TL-42 | 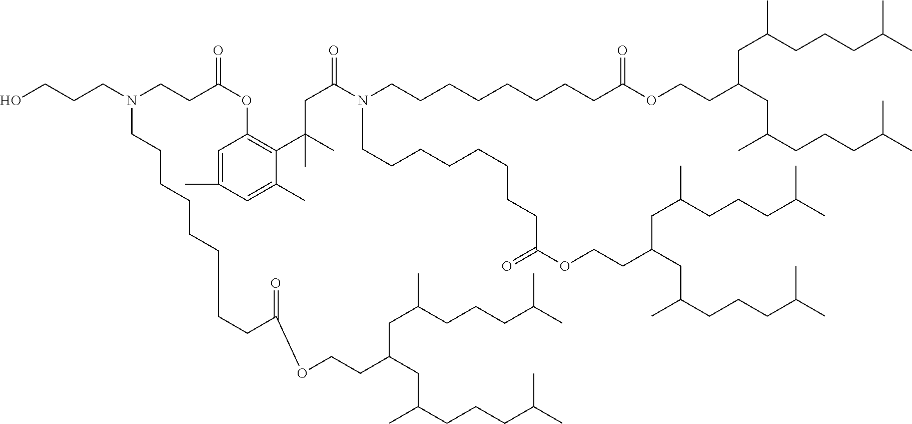 [SMILES: Cc1cc(C)c(C(C)(C)CC(=O)N(CCCCCCCCC(=O)OCCC(CC(C)CCCC(C)C)CC(C)CCCC(C)C)CCCCCCCCC(=O)OCCC(CC(C)CCCC(C)C)CC(C)CCCC(C)C)c(OC(=O)CCN(CCCO)CCCCCCCCC(=O)OCCC(CC(C)CCCC(C)C)CC(C)CCCC(C)C)c1] |
| TL-43 | 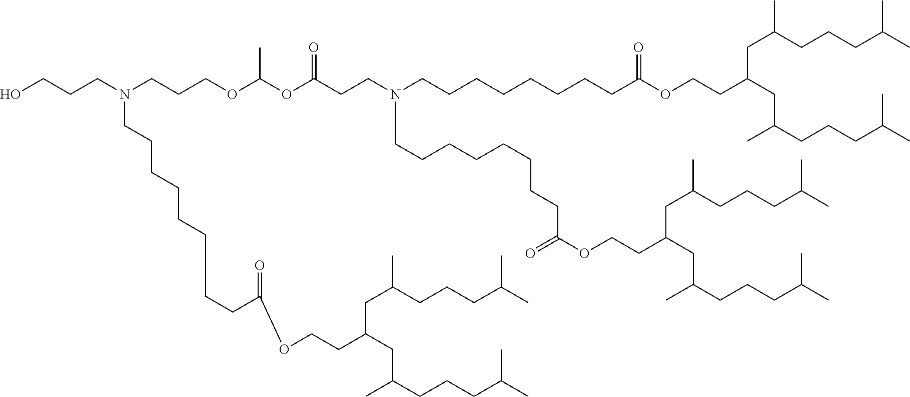 [SMILES: CC(C)CCCC(C)CC(CCOC(=O)CCCCCCCCN(CCCO)CCCOC(C)OC(=O)CCN(CCCCCCCCC(=O)OCCC(CC(C)CCCC(C)C)CC(C)CCCC(C)C)CCCCCCCCC(=O)OCCC(CC(C)CCCC(C)C)CC(C)CCCC(C)C)CC(C)CCCC(C)C] |
| TL-44 | 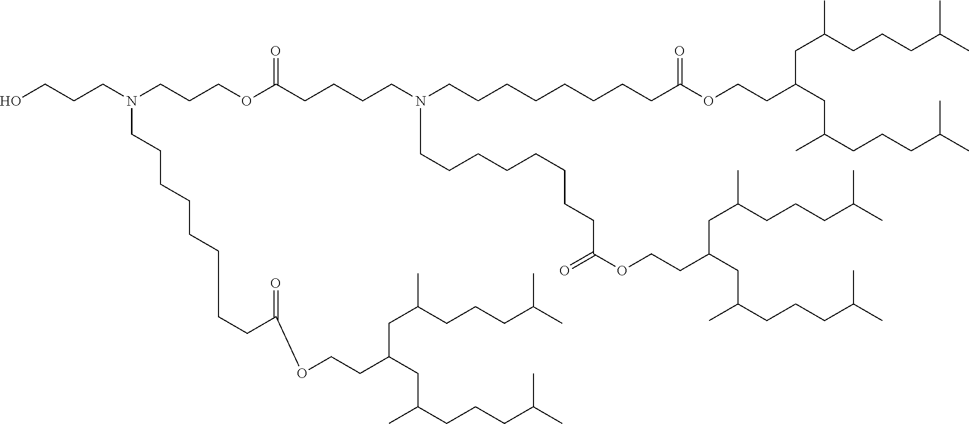 [SMILES: CC(C)CCCC(C)CC(CCOC(=O)CCCCCCCCN(CCCCCCCCC(=O)OCCC(CC(C)CCCC(C)C)CC(C)CCCC(C)C)CCCCC(=O)OCCCN(CCCO)CCCCCCCCC(=O)OCCC(CC(C)CCCC(C)C)CC(C)CCCC(C)C)CC(C)CCCC(C)C] |
| TL-45 | 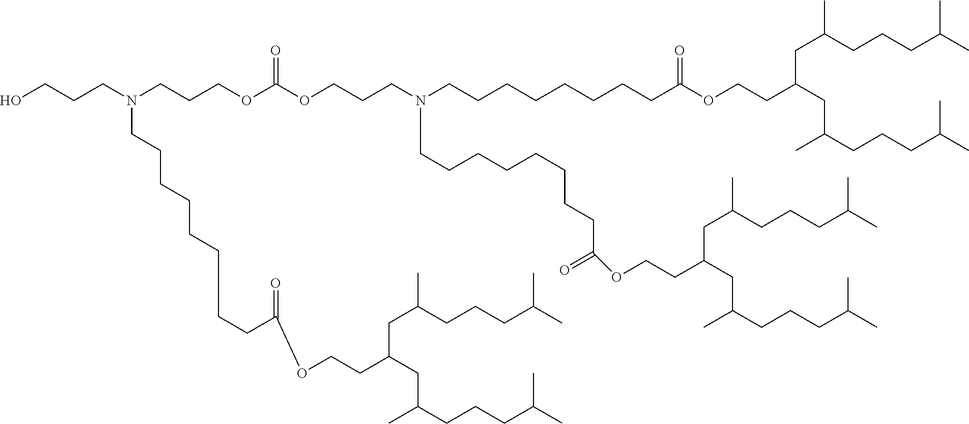 [SMILES: CC(C)CCCC(C)CC(CCOC(=O)CCCCCCCCN(CCCO)CCCOC(=O)OCCCN(CCCCCCCCC(=O)OCCC(CC(C)CCCC(C)C)CC(C)CCCC(C)C)CCCCCCCCC(=O)OCCC(CC(C)CCCC(C)C)CC(C)CCCC(C)C)CC(C)CCCC(C)C] |
| TL-46 | 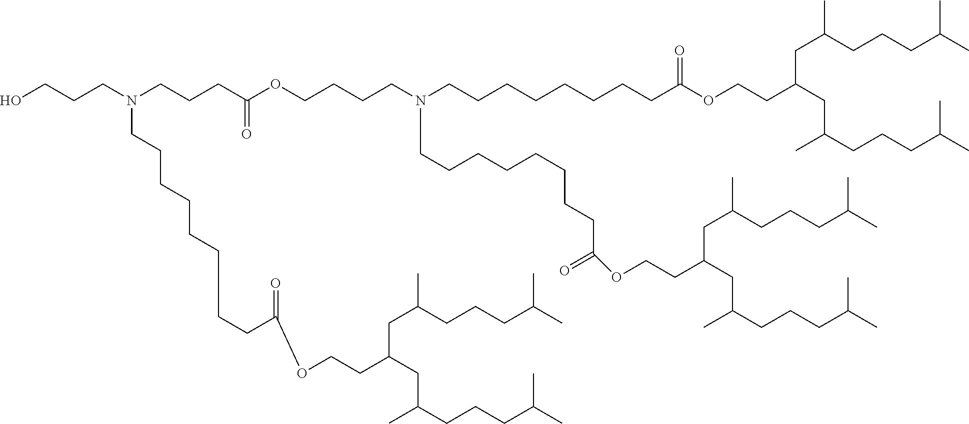 [SMILES: CC(C)CCCC(C)CC(CCOC(=O)CCCCCCCCN(CCCCCCCCC(=O)OCCC(CC(C)CCCC(C)C)CC(C)CCCC(C)C)CCCCOC(=O)CCCN(CCCO)CCCCCCCCC(=O)OCCC(CC(C)CCCC(C)C)CC(C)CCCC(C)C)CC(C)CCCC(C)C] |
| TL-47 | 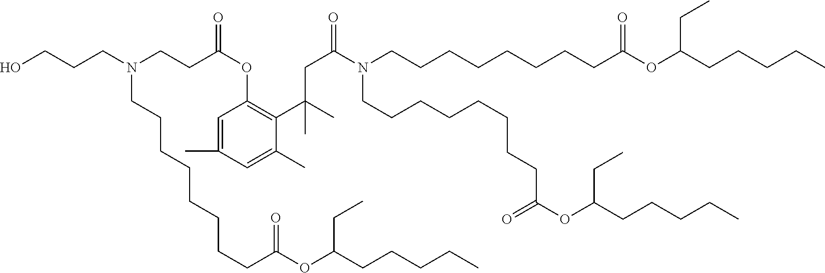 [SMILES: CCCCCC(CC)OC(=O)CCCCCCCCN(CCCO)CCC(=O)Oc1cc(C)cc(C)c1C(C)(C)CC(=O)N(CCCCCCCCC(=O)OC(CC)CCCCC)CCCCCCCCC(=O)OC(CC)CCCCC] |
| TL-48 | 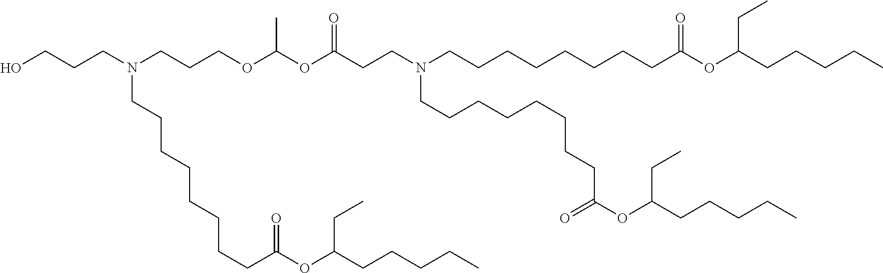 [SMILES: CCCCCC(CC)OC(=O)CCCCCCCCN(CCCO)CCCOC(C)OC(=O)CCN(CCCCCCCCC(=O)OC(CC)CCCCC)CCCCCCCCC(=O)OC(CC)CCCCC] |
| TL-49 | 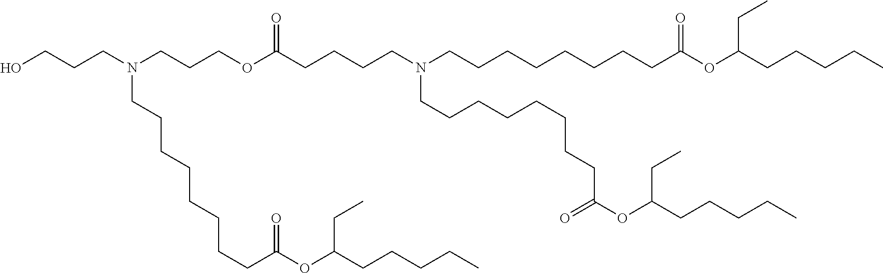 [SMILES: CCCCCC(CC)OC(=O)CCCCCCCCN(CCCCCCCCC(=O)OC(CC)CCCCC)CCCCC(=O)OCCCN(CCCO)CCCCCCCCC(=O)OC(CC)CCCCC] |
| TL-50 | 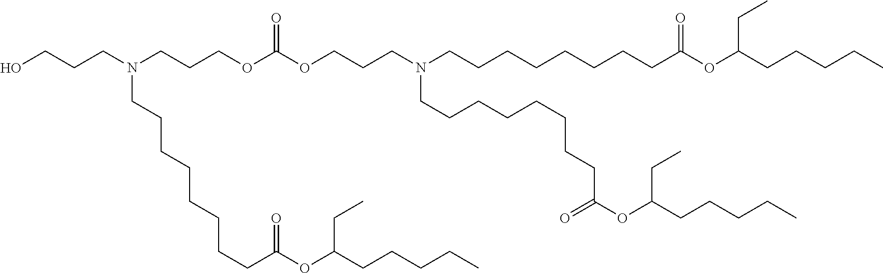 [SMILES: CCCCCC(CC)OC(=O)CCCCCCCCN(CCCO)CCCOC(=O)OCCCN(CCCCCCCCC(=O)OC(CC)CCCCC)CCCCCCCCC(=O)OC(CC)CCCCC] |
| TL-51 | 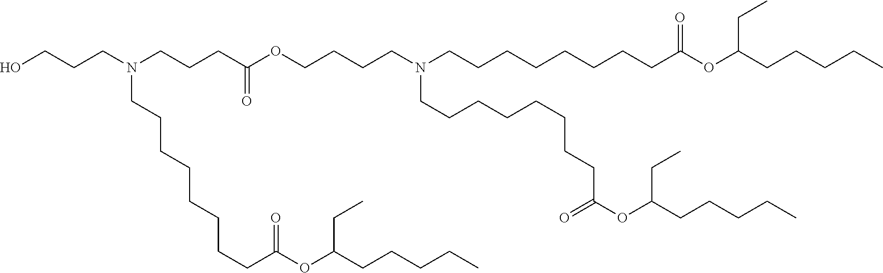 [SMILES: CCCCCC(CC)OC(=O)CCCCCCCCN(CCCCCCCCC(=O)OC(CC)CCCCC)CCCCOC(=O)CCCN(CCCO)CCCCCCCCC(=O)OC(CC)CCCCC] |
| TL-52 | 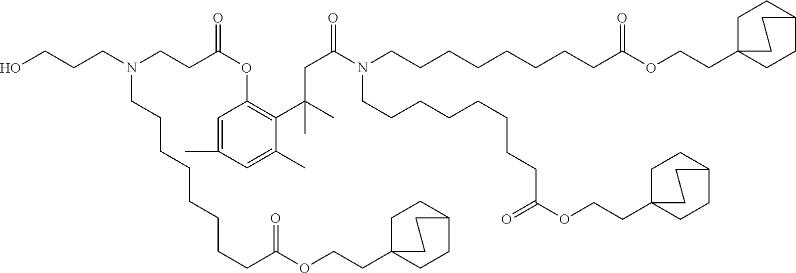 [SMILES: Cc1cc(C)c(C(C)(C)CC(=O)N(CCCCCCCCC(=O)OCCC23CCC(CC2)CC3)CCCCCCCCC(=O)OCCC23CCC(CC2)CC3)c(OC(=O)CCN(CCCO)CCCCCCCCC(=O)OCCC23CCC(CC2)CC3)c1] |
| TL-53 | 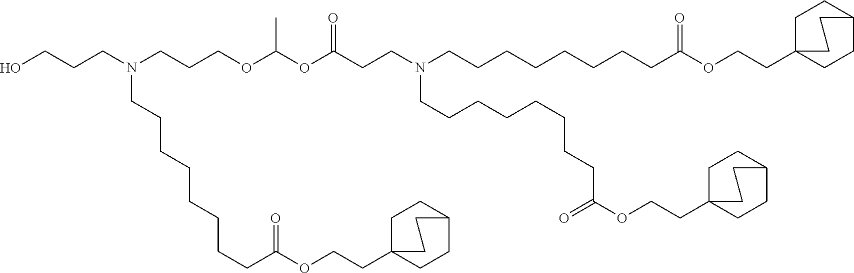 [SMILES: CC(OCCCN(CCCO)CCCCCCCCC(=O)OCCC12CCC(CC1)CC2)OC(=O)CCN(CCCCCCCCC(=O)OCCC12CCC(CC1)CC2)CCCCCCCCC(=O)OCCC12CCC(CC1)CC2] |
| TL-54 | 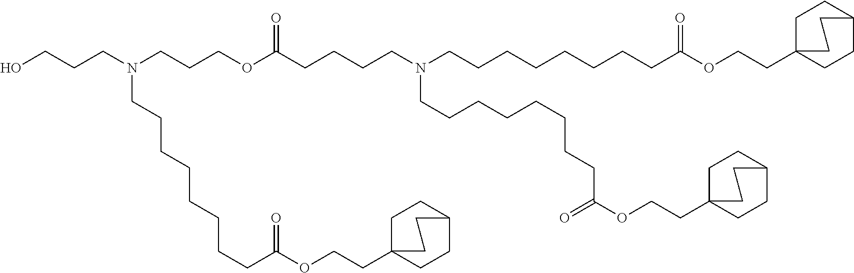 [SMILES: O=C(CCCCN(CCCCCCCCC(=O)OCCC12CCC(CC1)CC2)CCCCCCCCC(=O)OCCC12CCC(CC1)CC2)OCCCN(CCCO)CCCCCCCCC(=O)OCCC12CCC(CC1)CC2] |
| TL-55 | 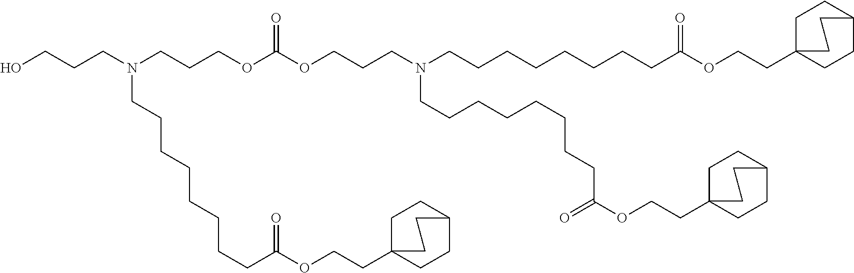 [SMILES: O=C(CCCCCCCCN(CCCO)CCCOC(=O)OCCCN(CCCCCCCCC(=O)OCCC12CCC(CC1)CC2)CCCCCCCCC(=O)OCCC12CCC(CC1)CC2)OCCC12CCC(CC1)CC2] |
| TL-56 | 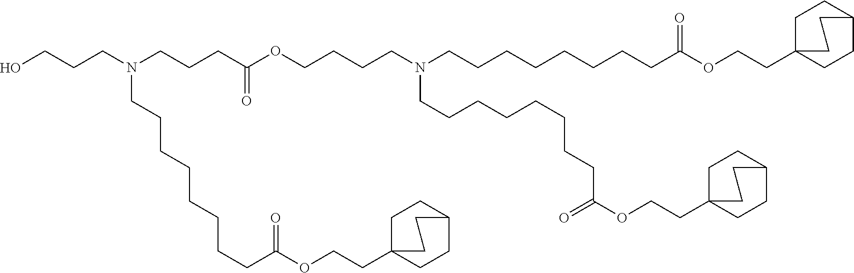 [SMILES: O=C(CCCN(CCCO)CCCCCCCCC(=O)OCCC12CCC(CC1)CC2)OCCCCN(CCCCCCCCC(=O)OCCC12CCC(CC1)CC2)CCCCCCCCC(=O)OCCC12CCC(CC1)CC2] |

In some embodiments, ionizable lipids of the present disclosure are selected from any lipid in Tables (I-A), (I-B), (I-C), (I-D), (I-E), (I-F), (I-G), (I-H), (I-I), (I-J), (I-K), (I-L), or (I-K) above, an enantiomer thereof, or any mixture of enantiomers thereof, or a pharmaceutically acceptable salt of any of the aforementioned.

In some embodiments, an ionizable lipid is described in US patent publication number 20190321489. In some embodiments, an ionizable lipid is described in international patent publication WO 2010/053572, incorporated herein by reference. In some embodiments, an ionizable lipid is C₁₂-200, described at paragraph [00225] of WO 2010/053572.

Several ionizable lipids have been described in the literature, many of which are commercially available. In certain embodiments, such ionizable lipids are included in the transfer vehicles described herein. Other suitable cationic lipids include, for example, ionizable cationic lipids as described in U.S. provisional patent application 61/617,468, filed Mar. 29, 2012 (incorporated herein by reference), such as, e.g., (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002), C12-200 (described in WO 2010/053572), 2-(2,2-di((9Z,12Z)-octadeca-9,12-dien-1-yl)-1,3-dioxolan-4-yl)-N,N-dimethylethanamine (DLinKC2-DMA)) (See, WO 2010/042877; Semple et al., Nature Biotech. 28:172-176 (2010)), 2-(2,2-di((9Z,2Z)-octadeca-9,12-dien-1-yl)-1,3-dioxolan-4-yl)-N,N-dimethylethanamine (DLin-KC2-DMA), (3S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), (15Z,18 Z)-N,N-dimethyl-6-((9Z,12Z)-octadeca-9,12-dien-1-yl)tetracosa-5,15,18-trien-1-amine (HGT5002), 5-carboxyspermylglycine-dioctadecylamide (DOGS), 2,3-dioleyloxy-N-[2(spermine-carboxamido)ethyl]-N,N-dimethyl-1-propanaminium (DOSPA) (Behr et al. Proc. Nat.'l Acad. Sci. 86, 6982 (1989); U.S. Pat. Nos. 5,171,678; 5,334,761), 1,2-Dioleoyl-3-Dimethylammonium-Propane (DODAP), 1,2-Dioleoyl-3-Trimethylammonium-Propane or (DOTAP). Contemplated ionizable lipids also include 1,2-distcaryloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylamninopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA) or GL67, or mixtures thereof.

Also contemplated are cationic lipids such as dialkylamino-based, imidazole-based, and guanidinium-based lipids. For example, also contemplated is the use of the ionizable lipid (3S,10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), as disclosed in International Application No. PCT/US2010/058457, incorporated herein by reference.

Also contemplated are ionizable lipids such as the dialkylamino-based, imidazole-based, and guanidinium-based lipids.

In some embodiments, an ionizable lipid is described by US patent publication number 20190314284.

The ionizable lipids include those disclosed in international patent application PCT/US2019/025246, and US patent publications 2017/0190661 and 2017/0114010, incorporated herein by reference in their entirety.

In some embodiments, an ionizable lipid is as described in international patent application PCT/US2019/015913.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include, e.g., hydroxyl, amino, mercapto, and carboxylic acid. Suitable protecting groups for hydroxyl include, e.g., trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino, and guanidino include, e.g., t-butoxycarbonyl, benzyloxycarbonyl, and the like. Suitable protecting groups for mercapto include, e.g., —C(O)—R″ (where R″ is alkyl, aryl, or arylalkyl), p-methoxybenzyl, trityl, and the like. Suitable protecting groups for carboxylic acid include, e.g., alkyl, aryl, or arylalkyl esters. Protecting groups may be added or removed in accordance with standard techniques, which are known to one skilled in the art and as described herein. The use of protecting groups is described in detail in, e.g., Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

It will also be appreciated by those skilled in the art, although such protected derivatives of compounds described herein may not possess pharmacological activity as such, they may be administered to a mammal and thereafter metabolized in the body to form compounds described herein which are pharmacologically active. Such derivatives may therefore be described as prodrugs. All prodrugs of compounds described herein are included within the scope of the present disclosure.

Furthermore, all compounds described herein which exist in free base or acid form can be converted to their pharmaceutically acceptable salts by treatment with the appropriate inorganic or organic base or acid by methods known to one skilled in the art. Salts of the compounds described herein can also be converted to their free base or acid form by standard techniques.

Preparation methods for the above compounds and compositions are described herein below and/or known in the art. It is understood that one skilled in the art may be able to make these compounds by similar methods or by combining other methods known to one skilled in the art.

a. Amine Lipids

In certain embodiments, transfer vehicle compositions for the delivery of circular RNA comprise an amine lipid. In certain embodiments, an ionizable lipid is an amine lipid.

In some embodiments, an amine lipid is described in international patent application PCT/US2018/053569. In some embodiments, the amine lipid is Lipid E of WO 2022/261490 and WO 2023/056033, which is (9Z, 12Z)-3-((4,4-bis(octyloxy)butanoyl)oxy)-2-((((3-(diethylamino)propoxy)carbonyl)oxy)methyl)propyl octadeca-9, 12-dienoate. Lipid E may be synthesized according to WO2015/095340 (e.g., pp. 84-86). In certain embodiments, the amine lipid is an equivalent to Lipid E. In certain embodiments, an amine lipid is an analog of Lipid E.

Amine lipids and other biodegradable lipids suitable for use in the transfer vehicles, e.g., lipid nanoparticles, described herein are biodegradable in vivo. The amine lipids described herein have low toxicity (e.g., are tolerated in animal models without adverse effect in amounts of greater than or equal to 10 mg/kg). In certain embodiments, transfer vehicles composing an amine lipid include those where at least 75% of the amine lipid is cleared from the plasma within 8, 10, 12, 24, or 48 hours, or 3, 4, 5, 6, 7, or 10 days.

Biodegradable lipids include, for example, the biodegradable lipids of WO2017/173054, WO2015/095340, and WO2014/136086.

Lipid clearance may be measured by methods known by persons of skill in the art. See, for example, Maier, M. A., et al. Biodegradable Lipids Enabling Rapidly Eliminated Lipid Nanoparticles for Systemic Delivery of RNAi Therapeutics. Mol. Ther. 2013, 21(8), 1570-78.

Transfer vehicle compositions comprising an amine lipid can lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which a circRNA is cleared from the blood, serum, or plasma. In some embodiments, the clearance rate is the rate at which transfer vehicles are cleared from the blood, serum, or plasma. In some embodiments, the clearance rate is the rate at which transfer vehicles are cleared from a tissue, such as liver tissue or spleen tissue. In certain embodiments, a high rate of clearance leads to a safety profile with no substantial adverse effects. The amine lipids and biodegradable lipids may reduce transfer vehicle accumulation in circulation and in tissues. In some embodiments, a reduction in transfer vehicle accumulation in circulation and in tissues leads to a safety profile with no substantial adverse effects.

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood, where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., into tumors. See, e.g., WO2014/136086.

b. Lipids Containing A Disulfide Bond

In some embodiments, the ionizable lipid is described in U.S. Pat. No. 9,708,628.

In some embodiments, the lipid may have an —S—S— (disulfide) bond. The production method for such a compound includes, for example, a method including producing
R^{3a}—(Y^{a}—R^{2a})n^{a}-X^{a}—R^{1a}—SH, and
R^{3b}—(Y^{b}—R^{2b})n^{b}-X^{b}—R^{1b}—SH, and
   subjecting them to oxidation (coupling) to give a compound containing —S—S—, a method including sequentially bonding necessary parts to a compound containing an —S—S— bond to finally obtain the compound and the like. Preferred is the latter method.

An example of the latter method is described on pages 470-472 of WO 2022/261490, which is incorporated by reference herein in its entirety.

Exemplary lipids containing a disulfide bond are described in WO 2022/261490, including the lipid represented by structure (XXII) described therein at pages 459-469 and structures 1-15 of Table 15b, which are incorporated by reference herein in its entirety. WO 2023/056033 is also incorporated by reference herein in its entirety.

c. Further Exemplary Lipids or Lipid-Like Compounds

In some embodiments, an ionizable lipid is described in U.S. Pat. No. 9,765,022.

In some embodiments, a lipid-like compound is represented by structure (XXIII), and is described in WO 2022/261490 and WO 2023/056033, including the lipid compounds comprising the exemplary hydrophilic heads, hydrophobic tails, linkers, and exemplary lipid-like compounds described therein.

As described therein, the lipid-like compounds of structure XXIII of WO 2022/261490 and WO 2023/056033 and other lipid-like compounds can be prepared by methods well known the art. See Wang et al., ACS Synthetic Biology, 1, 403-07 (2012); Manoharan, et al., International Patent Application Publication WO 2008/042973; and Zugates et al., U.S. Pat. No. 8,071,082. WO 2022/261490 and WO 2023/056033 describe an exemplary route of synthesis of the lipid-like compounds, and other suitable starting materials and routes of synthesis known in the art. See, for example, R. Larock, Comprehensive Organic Transformations (2nd Ed., VCH Publishers 1999); P. G. M. Wuts and T. W. Greene, Greene's Protective Groups in Organic Synthesis (4th Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2nd ed., John Wiley and Sons 2009) and subsequent editions thereof. Certain lipid-like compounds may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

As mentioned above, these lipid-like compounds are useful for delivery of pharmaceutical agents. They can be preliminarily screened for their efficacy in delivering pharmaceutical agents by an in vitro assay and then confirmed by animal experiments and clinic trials. Other methods will also be apparent to those of ordinary skill in the art.

The above described complexes can be prepared using procedures described in publications such as Wang et al., ACS Synthetic Biology, 1, 403-07 (2012). Generally, they are obtained by incubating a lipid-like compound and a pharmaceutical agent in a buffer such as a sodium acetate buffer or a phosphate buffered saline (“PBS”).

d. Hydrophilic Groups

In certain embodiments, the selected hydrophilic functional group or moiety may alter or otherwise impart properties to the compound or to the transfer vehicle of which such compound is a component (e.g., by improving the transfection efficiencies of a lipid nanoparticle of which the compound is a component). For example, the incorporation of guanidinium as a hydrophilic head-group in the compounds disclosed herein may promote the fusogenicity of such compounds (or of the transfer vehicle of which such compounds are a component) with the cell membrane of one or more target cells, thereby enhancing, for example, the transfection efficiencies of such compounds. It has been hypothesized that the nitrogen from the hydrophilic guanidinium moiety forms a six-membered ring transition state which grants stability to the interaction and thus allows for cellular uptake of encapsulated materials. (Wender, et al., Adv. Drug Del. Rev. (2008) 60: 452-472.) Similarly, the incorporation of one or more amino groups or moieties into the disclosed compounds (e.g., as a head-group) may further promote disruption of the endosomal/lysosomal membrane of the target cell by exploiting the fusogenicity of such amino groups. This is based not only on the pKa of the amino group of the composition, but also on the ability of the amino group to undergo a hexagonal phase transition and fuse with the target cell surface, i.e. the vesicle membrane. (Koltover, et al. Science (1998) 281: 78-81.) The result is believed to promote the disruption of the vesicle membrane and release of the lipid nanoparticle contents into the target cell.

Similarly, in certain embodiments the incorporation of, for example, imidazole as a hydrophilic head-group in the compounds disclosed herein may serve to promote endosomal or lysosomal release of, for example, contents that are encapsulated in a transfer vehicle (e.g., lipid nanoparticle) of the present disclosure. Such enhanced release may be achieved by one or both of a proton-sponge mediated disruption mechanism and/or an enhanced fusogenicity mechanism. The proton-sponge mechanism is based on the ability of a compound, and in particular a functional moiety or group of the compound, to buffer the acidification of the endosome. This may be manipulated or otherwise controlled by the pKa of the compound or of one or more of the functional groups comprising such compound (e.g., imidazole). Accordingly, in certain embodiments the fusogenicity of, for example, the imidazole-based compounds disclosed herein (e.g., HGT4001 and HGT4004) are related to the endosomal disruption properties, which are facilitated by such imidazole groups, which have a lower pKa relative to other traditional ionizable lipids. Such endosomal disruption properties in turn promote osmotic swelling and the disruption of the liposomal membrane, followed by the transfection or intracellular release of the polynucleotide materials loaded or encapsulated therein into the target cell. This phenomenon can be applicable to a variety of compounds with desirable pKa profiles in addition to an imidazole moiety. Such embodiments also include multi-nitrogen based functionalities such as polyamines, poly-peptide (histidine), and nitrogen-based dendritic structures.

Exemplary ionizable and/or cationic lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004 143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO20 12/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety. International patent application WO 2019/131770 is also incorporated herein by reference in its entirety.

### B. Stabilizing Lipids (e.g., PEG Lipids)

A stabilizing lipid or surface stabilizing lipid may be used to enhance the structure of the LNP. A stabilizing lipid as contemplated herein may be a polyethylene glycol (PEG)-modified phospholipid.

The use and inclusion of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C₈ PEG-2000 ceramide) in the liposomal and pharmaceutical compositions described herein is contemplated, preferably in combination with one or more of the compounds and lipids disclosed herein. Contemplated PEG-modified lipids include, but are not limited to, a polyethylene glycol chain of up to 5 kDa in length covalently attached to a lipid with alkyl chain(s) of C6-C20 length. In some embodiments, the PEG-modified lipid employed in the compositions and methods described herein is 1,2-dimyristoyl-sn-glycerol, methoxypolyethylene Glycol (2000 MW PEG) “DMG-PEG2000.” The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids may comprise a molar ratio from about 0% to about 20%, about 0.5% to about 20%, about 1% to about 15%, about 4% to about 10%, or about 2% of the total lipid present in a liposomal lipid nanoparticle.

In some embodiments, the lipid moiety of the PEG-lipids includes those having lengths of from about C14 to about C22, such as from about C14 to about C16. In some embodiments, a PEG moiety, for example a mPEG-NH2, has a size of about 1000, about 2000, about 5000, about 10,000, about 15,000 or about 20,000 daltons

In an embodiment, a PEG-modified lipid is described in International Pat. Appl. No. PCT/US2019/015913, which is incorporated herein by reference in their entirety. In an embodiment, a transfer vehicle comprises one or more PEG-modified lipids.

Non-limiting examples of PEG-modified lipids include PEG-modified phosphatidylethanolamines and phosphatidic acids, PEG-ceramide conjugates (e.g., PEG-CerC14 or PEG-CerC20), PEG-modified dialkylamines and PEG-modified 1,2-diacyloxypropan-3-amines, PEG-modified diacylglycerols, PEG-modified dialkylglycerols, and mixtures thereof. In some further embodiments, a PEG-modified lipid may be, e.g., PEG-c-DOMG, PEG-DMG, PEG-DLPE, PEG-DMPE, PEG-DPPC, PEG-DSPE, PEG-DAG, PEG-S-DAG, PEG-PE, PEG-S-DMG, PEG-CER, PEG-dialkoxypropylcarbamate, PEG-OR, PEG-OH, PEG-c-DOMG, or PEG-1.

In some still further embodiments, the PEG-modified lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the lipid nanoparticles described herein can comprise a lipid modified with a non-diffusible PEG. Non-limiting examples of non-diffusible PEGs include PEG-DSG and PEG-DSPE. In one embodiment, the lipid nanoparticles herein comprise PEG-DSPC.

In some embodiments the PEG-modified lipids are a modified form of PEG-DMG. PEG-DMG has the following structure:

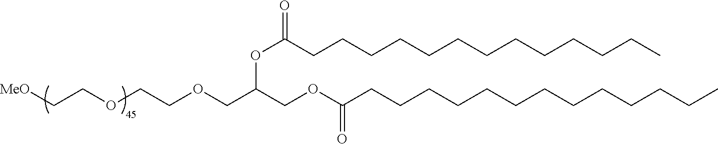

In some embodiments, the PEG lipid is a compound of Formula (P1):

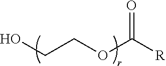

or a salt or isomer thereof, wherein:
r is an integer between 1 and 100;
R is C10-40 alkyl, C10-40 alkenyl, or C10-40 alkynyl; and optionally one or more methylene groups of R are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC(O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS(O)—, —S(O)O—, —OS(O)O—, —OS(O)2-, —S(O)2O—, —OS(O)2O—, —N(RN)S(O)—, —S(O)N(RN)—, —N(RN)S(O)N(RN)—, —OS(O)N(RN)—, —N(RN)S(O)O—, —S(O)2-, —N(RN)S(O)2-, —S(O)₂N(RN)—, —N(RN)S(O)₂N(RN)—, —OS(O)₂N(RN)—, or —N(RN)S(O)2O—; and
each instance of RN is independently hydrogen, C1-6 alkyl, or a nitrogen protecting group.

For example, R is C17 alkyl. For example, the PEG lipid is a compound of Formula (P1-a):

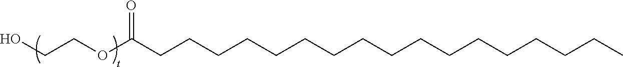

or a salt or isomer thereof, wherein r is an integer between 1 and 100.

In some embodiments the PEG-modified lipids are a modified form of PEG-C₁₈, or PEG-1. PEG-1 has the following structure:

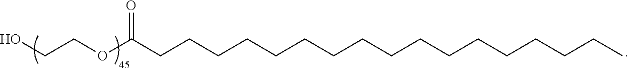

PEG-lipids are known in the art, such as those described in U.S. Pat. No. 8,158,601 and International Pat. Publ. No. WO2015/130584 A2, which are incorporated herein by reference in their entirety. In one embodiment, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In some embodiments, an LNP comprises one, two or more PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DSG-PEG2k, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety.

In some embodiments, the LNP comprises a PEGylated lipid substitute in place of the PEGylated lipid. All embodiments disclosed herein that contemplate a PEGylated lipid should be understood to also apply to PEGylated lipid substitutes. In some embodiments, the LNP comprises a polysarcosine-lipid conjugate, such as those disclosed in US 2022/0001025 A1, which is incorporated by reference herein in its entirety. In some embodiments the LNP comprises a polyoxazoline-lipid conjugate, such as those disclosed in US 2022/0249695 A1, which is incorporated by reference herein in its entirety.

### Series “PL”

In some embodiments, the LNP comprises a PEGylated lipid disclosed and described in PCT Application WO 2024/044728 A1, which is incorporated by reference herein, in its entirety. In certain embodiments, the PEGylated lipid is a lipid of any one of formulas PL-I′, PL-I″, PL-I, PL-Ia, PL-Ib, PL-Iaa, PL-Iab, PL-Iac, PL-Iad, PL-Iae, PL-Iaf, PL-Iag, PL-Iah, PL-Iba, PL-Ibb, PL-Ibc, PL-Ibd, PL-Ibe, PL-Ibf, PL-Ibg, PL-Ibh, PL-Ica, PL-Icb, PL-Icc, PL-Icd, PL-Id PL-Ie, PL-If, PL-Ig, PL-Ih, PL-Ii, PL-Iha, PL-Ihb, PL-Ihc, PL-Ihd, PL-Iia, PL-Iib, PL-Iic, PL-Iid, PL-Ij, PL-Ik, L-Il, PL-Im, PL-In, PL-Io, PL-Ip, PL-Iq, PL-Ioa, PL-Iob, PL-Ioc, PL-Iod, PL-Ioe, PL-Iof, PL-Iog, PL-Ioh, PL-Ipa, PL-Ipb, PL-Ipc, PL-Ipd, PL-Ipe, PL-Ipf, PL-Ipg, PL-Iph, PL-Iqa, PL-Iqb, PL-Iqc, PL-Iqd, PL-Ir, PL-Is, PL-It, PL-Iu, PL-Iv, PL-Iw, PL-Iva, PL-Ivb, PL-Ivc, PL-Ivd, PL-Iwa, PL-Iwb, PL-Iwc, PL-Iwd, PL-Ix, PL-Ixx, PL-Iy, PL-Iyy, PL-Iyyy, PL-Iz, PL-Izz, PL-Izzz, PL-II′, PL-II″, PL-II, PL-IIc, PL-IId, PL-IIe, PL-IIf, PL-IIg, PL-IIh, PL-IIa, PL-IIb, PL-Ilk, PL-IIm or PL-IIn.

In some embodiments, the PEGylated lipid is a compound of formula PL-I′:

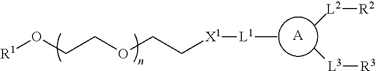

or a pharmaceutically acceptable salt thereof, wherein:
A¹ is a saturated 5-6 membered carbocyclic ring or a saturated 5-6 membered heterocyclic ring containing 1 or 2 heteroatoms independently selected from nitrogen, oxygen, and sulfur, wherein the carbocyclic ring and heterocyclic ring are substituted with t occurrences of R⁴; X¹ is —N(H)—, —N(C₁₋₆ alkyl)-, —C₁₋₆ aliphatic-N(H)—, —C₁₋₆ aliphatic-N(C₁₋₆ alkyl)-, —O— or —C₁₋₆ aliphatic-O—;
L¹ is —C(O)(C₁₋₆ aliphatic)C(O)—N(R)—, —C(O)(C₁₋₆ aliphatic)-N(R)C(O)—, —C(O)(C₁₋₆ aliphatic)C(O)O—, —C(O)(C₁₋₆ aliphatic)C(O)—, —C(O)(C₁₋₆ aliphatic)C(O)OCH₂—, —C(O)(C₁₋₆ aliphatic)-, —C(O)(C₁₋₆ aliphatic)-N(R)—, or —C(O)—;
L² and L³ are independently a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁵)═N—, or —C(R₅)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently a straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x};
R⁴ is C₁₋₄ alkyl;
R⁵ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{x} is independently halogen, —CN, —OR, —SR, —C(O)R, —C(O)OR, or —OC(O)OR;
n is an integer from 10-75, inclusive;
m is 0, 1, 2, 3, or 4; and
t is 0, 1, or 2.

In some embodiments, the PEGylated lipid is a compound of formula PL-II′:

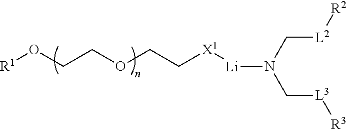

or a pharmaceutically acceptable salt thereof, wherein:
X¹ is —N(H)—, —N(C₁₋₆ alkyl)-, —C₁₋₆ aliphatic-N(H)—, —C₁₋₆ aliphatic-N(C₁₋₆ alkyl)-, —O— or —C₁₋₆ aliphatic-O—;
L¹ is —C(O)(C₁₋₆ aliphatic)C(O)—, —C(O)(C₁₋₆ aliphatic)-, or —C(O)—;
L² and L³ are a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁶)═N—, or —C(R₆)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x}; R⁶ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{x} is independently halogen, —CN, —OR, —SR, —C(O)R, —C(O)OR, or OC(O)OR; n is an integer from 10-75, inclusive; and m is 0, 1, 2, 3, or 4.

In some embodiments, the PEGylated lipid compound is one of those shown in Table (I-X), or a pharmaceutically acceptable salt thereof.

TABLE (I-X)
Exemplary PEGylated Compounds

| Compound | |
|---|---|
| No. | Structure |
| PL-1 | 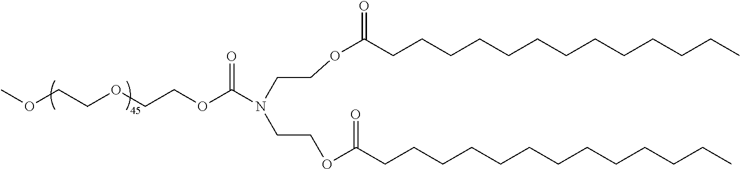 |
| PL-1A | 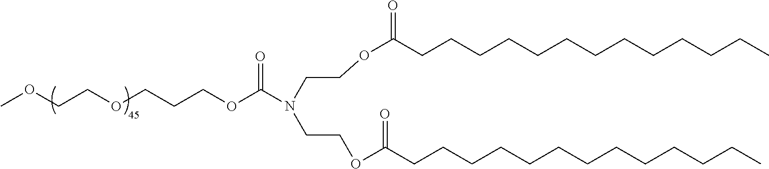 |
| PL-2 | 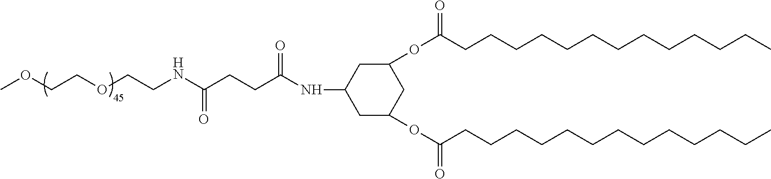 |
| PL-2A | 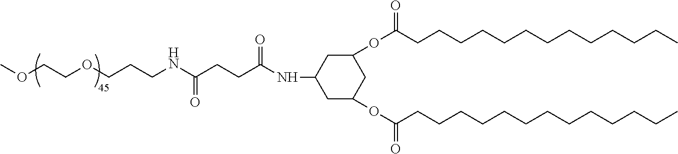 |
| PL-3 | 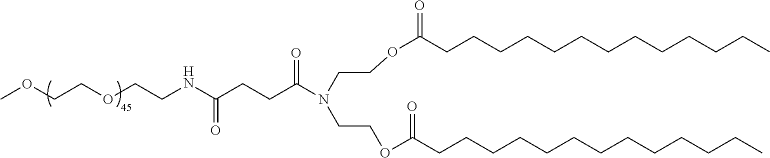 |
| PL-3A | 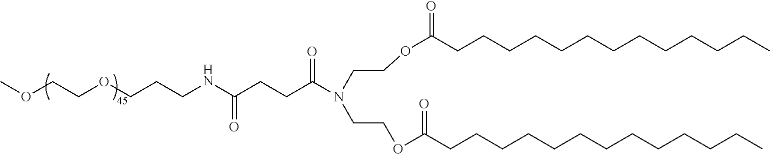 |
| PL-4 | 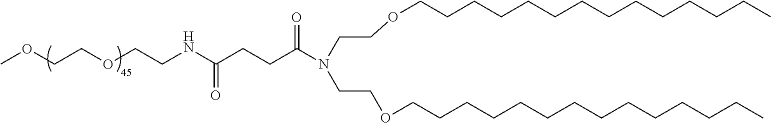 |
| PL-4A | 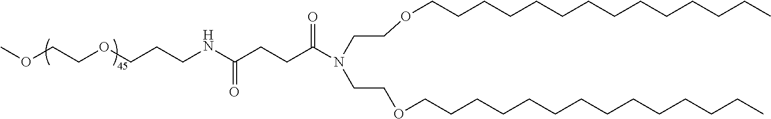 |
| PL-5 | 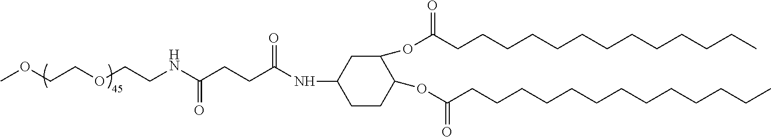 |
| PL-5A | 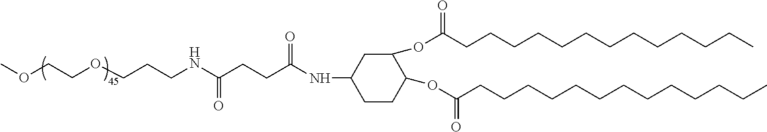 |
| PL-6 | 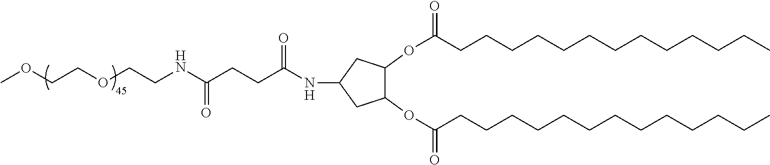 |
| PL-6A | 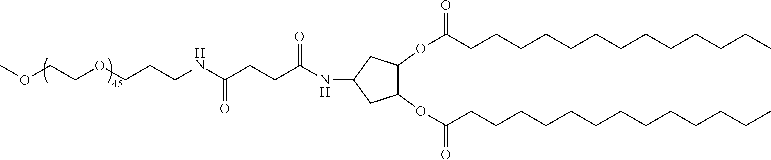 |
| PL-7 | 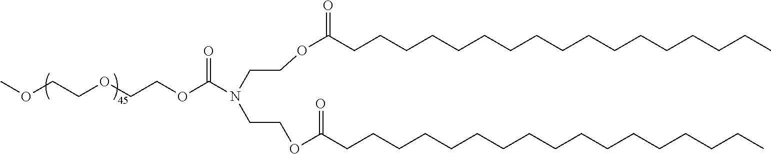 |
| PL-7A | 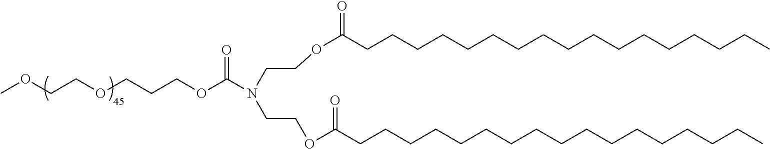 |
| PL-8 | 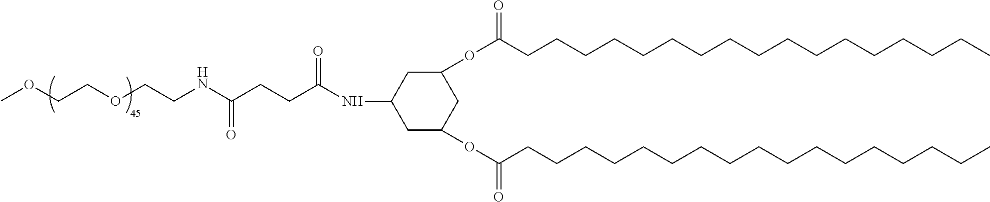 |
| PL-8A | 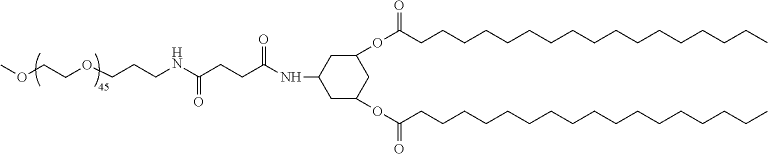 |
| PL-9 | 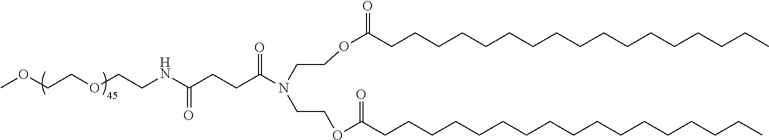 |
| PL-9A | 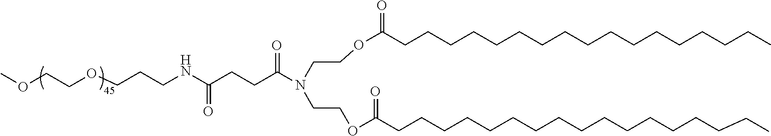 |
| PL-10 | 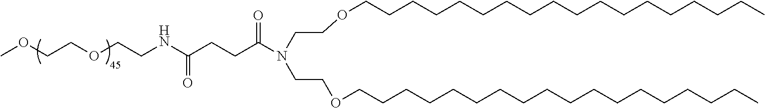 |
| PL-10A | 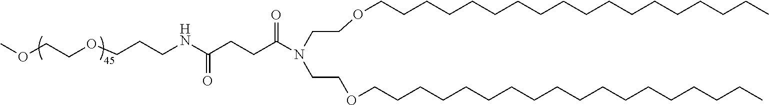 |
| PL-11 | 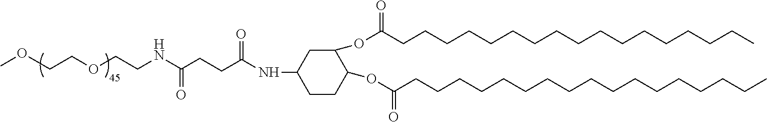 |
| PL-11A | 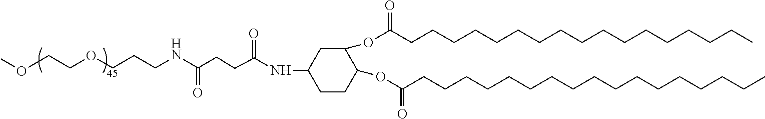 |
| PL-12 | 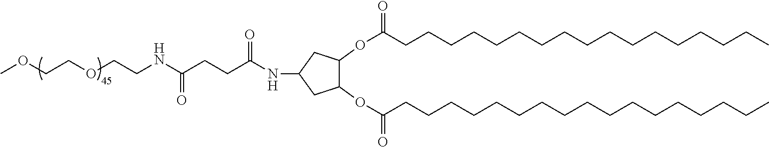 |
| PL-12A | 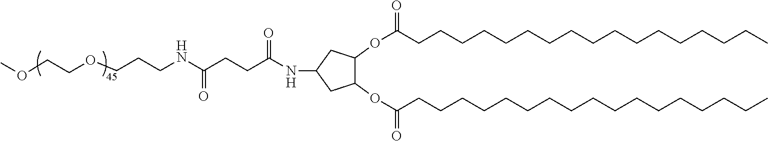 |
| PL-12B | 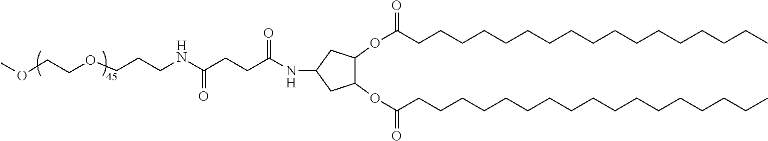 |
| PL-13 | 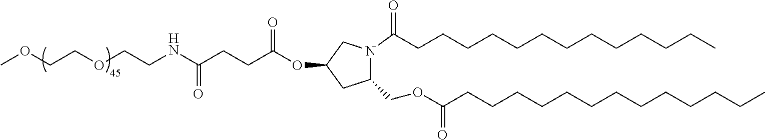 |
| PL-13A | 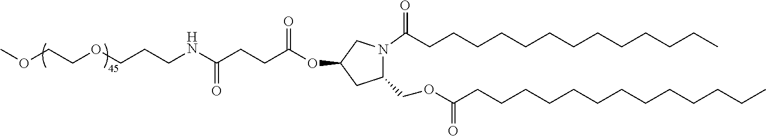 |
| PL-14 | 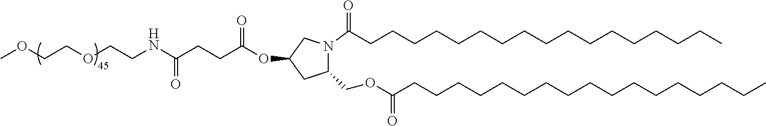 |
| PL-14A | 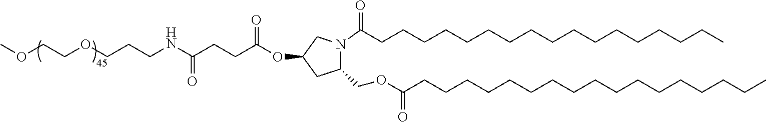 |
| PL-15 | 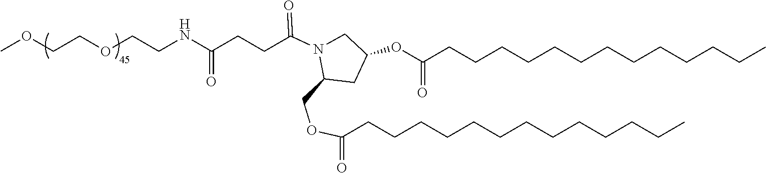 |
| PL-15A | 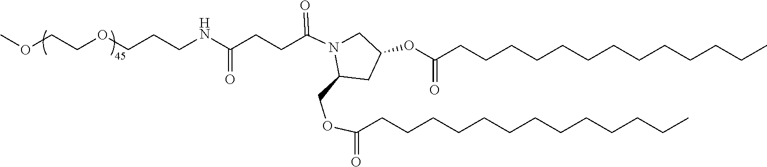 |
| PL-16 | 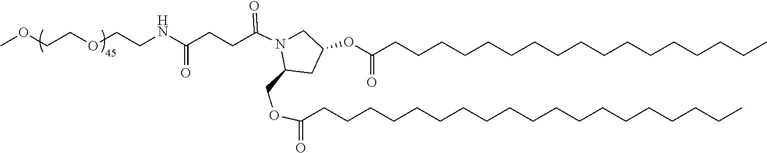 |
| PL-16A | 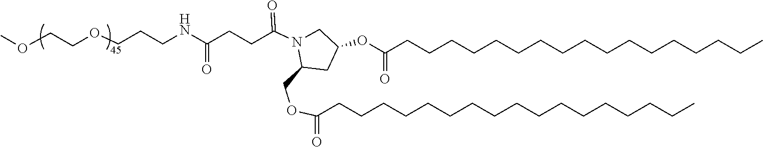 |

### C. Helper Lipids

In some embodiments, the transfer vehicle (e.g., LNP) described herein comprises one or more non-cationic helper lipids. In some embodiments, the helper lipid is a phospholipid. In some embodiments, the helper lipid is a phospholipid substitute or replacement. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the helper lipid is a 1,2-distearoyl-177-glycero-3-phosphocholine (DSPC) analog, a DSPC substitute, oleic acid, or an oleic acid analog.

In some embodiments, a helper lipid is a non-phosphatidyl choline (PC) zwitterionic lipid, a DSPC analog, oleic acid, an oleic acid analog, or a DSPC substitute.

In some embodiments, a helper lipid is described in PCT/US2018/053569. Helper lipids suitable for use in a lipid composition of the disclosure include, for example, a variety of neutral, uncharged or zwitterionic lipids. Such helper lipids are preferably used in combination with one or more of the compounds and lipids disclosed herein. Examples of helper lipids include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoylsn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-paimitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), paimitoyioieoyl phosphatidylcholine (POPC), lysophosphatidyl choline, dioleoyl phosphatidylethanol amine (DOPE) dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the helper lipid may be distearoylphosphatidylcholine (DSPC) or dimyristoyl phosphatidyl ethanolamine (DMPE). In another embodiment, the helper lipid may be distearoylphosphatidylcholine (DSPC). Helper lipids function to stabilize and improve processing of the transfer vehicles. Such helper lipids are preferably used in combination with other excipients, for example, one or more of the ionizable lipids disclosed herein. In some embodiments, when used in combination with an ionizable lipid, the helper lipid may comprise a molar ratio of 5% to about 90%, or about 10% to about 70% of the total lipid present in the lipid nanoparticle.

In certain embodiments, the helper lipid is a phospholipid selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin. In some embodiments, the helper lipid is a combination of 2, 3, 4, 5, 6, 7, 8, 9, 10, 11, or 12 of the above phospholipids. In some embodiments, an LNP comprises DSPC. In certain embodiments, an LNP comprises DOPE. In some embodiments, an LNP comprises both DSPC and DOPE.

In some embodiments, an LNP comprises a phospholipid selected from 1-pentadecanoyl-2-oleoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-palmitoyl-2-oleoyl-glycero-3-phosphocholine, 1-palmitoyl-2-linoleoyl-sn-glycero-3-phosphocholine, 1-palmitoyl-2-arachidonoyl-sn-glycero-3-phosphocholine, 1-palmitoyl-2-docosahexaenoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-stearoyl-2-oleoyl-sn-glycero-3-phosphocholine, 1-stearoyl-2-linoleoyl-sn-glycero-3-phosphocholine, 1-stearoyl-2-arachidonoyl-sn-glycero-3-phosphocholine, 1-stearoyl-2-docosahexaenoyl-sn-glycero-3-phosphocholine, 1-oleoyl-2-myristoyl-sn-glycero-3-phosphocholine, 1-oleoyl-2-palmitoyl-sn-glycero-3-phosphocholine, 1-oleoyl-2-stearoyl-sn-glycero-3-phosphocholine, 1-palmitoyl-2-acetyl-sn-glycero-3-phosphocholine, 1,2-dioleoyl-sn-glycero-3-phospho-(1′-myo-inositol-3′,4′-bisphosphate), 1,2-dioleoyl-sn-glycero-3-phospho-(1′-myo-inositol-3′,5′-bisphosphate), 1,2-dioleoyl-sn-glycero-3-phospho-(1′-myo-inositol-4′,5′-bisphosphate), 1,2-dioleoyl-sn-glycero-3-phospho-(1′-myo-inositol-3′,4′,5′-trisphosphate), 1,2-dioleoyl-sn-glycero-3-phospho-(1′-myo-inositol-3′-phosphate), 1,2-dioleoyl-sn-glycero-3-phospho-(1′-myo-inositol-4′-phosphate), 1,2-dioleoyl-sn-glycero-3-phospho-(1′-myo-inositol-5′-phosphate), 1,2-dioleoyl-sn-glycero-3-phospho-(1′-myo-inositol), 1,2-dioleoyl-sn-glycero-3-phospho-L-serine, and 1-(8Z-octadecenoyl)-2-palmitoyl-sn-glycero-3-phosphocholine. In some embodiments, the LNP comprises a phospholipid selected from DSPS (Distearoylphosphatidylserine), DSPG (1,2-distearoyl-sn-glycero-3-phospho-(1′-rac-glycerol)), DSPA (1,2-Distearoyl-sn-glycero-3-phosphate), diPhyPC (1,2-diphytanoyl-sn-glycero-3-phosphocholine), diPhy-diether-PC (1,2-di-O-phytanyl-sn-glycero-3-phosphocholine), diPhyPE (1,2-diphytanoyl-sn-glycero-3-phosphoethanolamine), diPhy-diether-PE (1,2-di-O-phytanyl-sn-glycero-3-phosphoethanolamine), diPhyPS (1,2-diphytanoyl-sn-glycero-3-phospho-L-serine), diPhyPG (1,2-diphytanoyl-sn-glycero-3-phospho-(1′-rac-glycerol)), diPhyPA (1,2-diphytanoyl-sn-glycero-3-phosphate), Egg PA (L-α-phosphatidic acid), and Soy PA (L-α-phosphatidic acid).

In some embodiments, the LNP comprises a phospholipid selected from 18:1 (Δ9-Cis) PE (DOPE), 18:0-18:1 PE (SOPE), C₁₆-18:1 PE, 16:0-18:1 PE (POPE), 18:1 BMP (S,R), 18:0-18:1 PC (SOPC), 16:0-18:1 PC (POPC), 4ME 16:0 Diether PE (4Me), 18:1 (Δ9-Trans) PE (DEPE), 16:1 PE (DPPE), and CL. In certain embodiments, the LNP comprises a phospholipid described or disclosed in Alvarez-Benedicto, et al. (Biomater. Sci., 2022, 10, 549) and Li, et al. (Asian Journal of Pharmaceutical Sciences, 2015, 10, 81-98).

In certain embodiments, the phospholipid is a sphingoid lipid or sphingolipid, such as, but not limited to sphingomyelin. As used herein, the terms “sphingoid lipid” and “sphingolipid” are meant to refer to a class of lipids containing a backbone comprising a sphingoid base. An exemplary sphingoid base is sphingosine. In certain embodiments, the LNP comprises a sphingolipid selected from Egg Sphingomyelin (Egg SM/ESM/(2S,3R,E)-3-hydroxy-2-palmitamidooctadec-4-en-1-yl(2-(trimethylammonio)ethyl) phosphate), Brain or Porcine Sphingomyelin (Brain SM/(2S,3R,E)-3-hydroxy-2-stearamidooctadec-4-en-1-yl(2-(trimethylammonio)ethyl) phosphate), Milk or Bovine Sphingomyelin (Milk SM/(2S,3R,E)-3-hydroxy-2-tricosanamidooctadec-4-en-1-yl(2-(trimethylammonio)ethyl) phosphate), 28:0 SM (N-octacosanoyl-D-erythro-sphingosylphosphorylcholine), 14:0 SM (N-myristoyl-D-erythro-sphingosylphosphorylcholine), 16:1 SM (N-palmitoleoyl-D-erythro-sphingosylphosphorylcholine), 12:0 Dihydro SM (N-lauroyl-D-erythro-sphinganylphosphorylcholine), Lyso SM (Sphingosylphosphorylcholine), Lyso SM (Sphingosylphosphorylcholine), Lyso SM (dihydro) (Sphinganine Phosphorylcholine), 24:1 SM (N-nervonoyl-D-erythro-sphingosylphosphorylcholine), 24:0 SM (N-lignoceroyl-D-erythro-sphingosylphosphorylcholine), 18:1 SM (N-oleoyl-D-erythro-sphingosylphosphorylcholine), 18:0 SM (N-stearoyl-D-erythro-sphingosylphosphorylcholine), 17:0 SM (N-heptadecanoyl-D-erythro-sphingosylphosphorylcholine), 16:0 SM (N-palmitoyl-D-erythro-sphingosylphosphorylcholine), 12:0 SM (N-lauroyl-D-erythro-sphingosylphosphorylcholine), 06:0 SM (N-hexanoyl-D-erythro-sphingosylphosphorylcholine), 02:0 SM (N-acetyl-D-erythro-sphingosylphosphorylcholine), 3-O-methyl Lyso SM (3-O-methyl-spingosylphosphorylcholine), 3-O-methyl-N-methyl Lyso SM (3-O-methyl-N-methyl-spingosylphosphorylcholine), and 3-N-methyl Lyso SM (3-N-methyl-spingosylphosphorylcholine).

In some embodiments, the LNP comprises a phospholipid comprising at least one constrained tail, such as those described by Gan, et al. (Bioeng Transl Med. 2020 September; 5(3): e10161.). In certain embodiments, the phospholipid is one selected from:

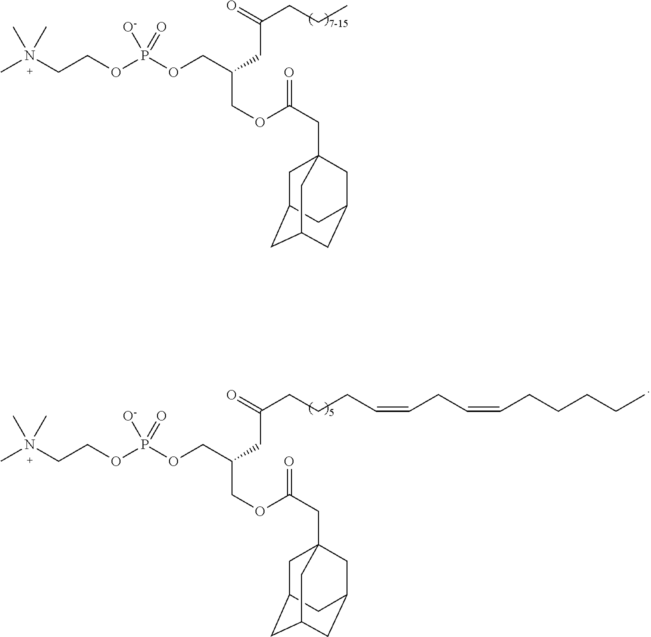

In some embodiments, the LNP comprises a phospholipid comprising a ceramide analogue having a triazole linkage, such as those described by Kim et al., Bioorg. Med. Chem. Lett., 17(16), 2007, 4584-4587.

In some embodiments, the LNP comprises a phospholipid disclosed in WO 2023/141470, which is incorporated by reference herein, in its entirety. In certain embodiments, the phospholipid is

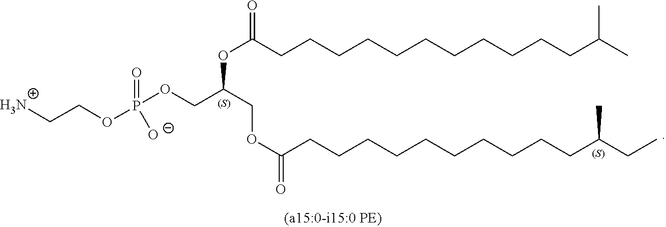

In some embodiments, the LNP comprises a phospholipid disclosed in WO 2022/040641, which is incorporated by reference herein, in its entirety.

In some embodiments, a phospholipid tail may be modified in order to promote endosomal escape as described in U.S. Application Publication 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, the LNP comprises a phospholipid disclosed in PCT Publication WO2023141470A2, which is incorporated by reference herein in its entirety. In certain embodiments, the LNP comprises a phospholipid of Formula (I) of PCT Publication WO2023141470A2, including but not limited to 2-ammonioethyl ((S)-3-(((S)-12-methyltetradecanoyl)oxy)-2-((13-methyltetradecanoyl)oxy)propyl) phosphate.

### D. Structural Lipids

The transfer vehicles described herein comprise one or more structural lipids. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid is an analog of cholesterol. In certain embodiments, the structural lipid includes cholesterol and a corticosteroid (such as, for example, prednisolone, dexamethasone, prednisone, and hydrocortisone), or a combination thereof.

In an embodiment, a structural lipid is described in international patent application PCT/US2019/015913.

In some embodiments, the structural lipid is a sterol (e.g., phytosterols or zoosterols). In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, or alpha-tocopherol. In certain embodiments, the structural lipid is cholesterol. In certain embodiments, the structural lipid is an analog of cholesterol. In certain embodiments, the structural lipid is alpha-tocopherol. In certain embodiments, the structural lipid is selected from the group consisting of, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and mixtures thereof. In some embodiments, the structural lipid is cholesterol. In some embodiments, the structural lipid is a cholesterol analogue disclosed by Patel, et al., Nat Commun., 11, 983 (2020), which is incorporated herein by reference in its entirety. In some embodiments, the structural lipid comprises cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

The transfer vehicles described herein comprise one or more structural lipids. Incorporation of structural lipids in a transfer vehicle, e.g., a lipid nanoparticle, may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, a transfer vehicle includes an effective amount of an immune cell delivery potentiating lipid, e.g., a cholesterol analog or an amino lipid or combination thereof, that, when present in a transfer vehicle, e.g., an lipid nanoparticle, may function by enhancing cellular association and/or uptake, internalization, intracellular trafficking and/or processing, and/or endosomal escape and/or may enhance recognition by and/or binding to immune cells, relative to a transfer vehicle lacking the immune cell delivery potentiating lipid. Accordingly, while not intending to be bound by any particular mechanism or theory, in one embodiment, a structural lipid or other immune cell delivery potentiating lipid of the disclosure binds to C1q or promotes the binding of a transfer vehicle comprising such lipid to C1q. Thus, for in vitro use of the transfer vehicles of the disclosure for delivery of a nucleic acid molecule to an immune cell, culture conditions that include C1q are used (e.g., use of culture media that includes serum or addition of exogenous C1q to serum-free media). For in vivo use of the transfer vehicles of the disclosure, the requirement for C1q is supplied by endogenous C1q.

In certain embodiments, the structural lipid is cholesterol. In certain embodiments, the structural lipid is an analog of cholesterol.

In some embodiments, a structural lipid of any one of the disclosed embodiments is selected from the structures set forth in Table 16 of WO 2022/261490 and Table 16 of WO 2023/056033, which are incorporated by reference herein in their entireties.

### E. Lipid Nanoparticle (LNP) Formulations

In certain embodiments, the transfer vehicle comprises a lipid. In certain embodiments, the transfer vehicle comprises an ionizable lipid. In certain embodiments, the transfer vehicle comprises an ionizable lipid in combination with other lipids, e.g., a structural lipid, and/or a PEG-modified lipid.

In certain embodiments, the transfer vehicle is a lipid nanoparticle (LNP), which may be capable of delivering the one or more RNA constructs (e.g., circular RNA and/or linear mRNA) to one or more target cells. In certain embodiments the transfer vehicle is capable of delivering the circular RNA construct to a human immune cell present in a human subject, such that the expression sequence encoding a binding molecule (e.g., CAR) is translated in the human immune cell and expressed on the surface of the human immune cell.

In certain embodiments, the transfer vehicles are prepared to encapsulate one or more materials or therapeutic agents (e.g., circular RNA and/or linear mRNA). The process of incorporating a desired therapeutic agent (e.g., circular RNA and/or linear mRNA) into a transfer vehicle is referred to herein as or “loading” or “encapsulating” (Lasic, et al., FEBS Lett., 312: 255-258, 1992). The transfer vehicle-loaded or -encapsulated materials (e.g., circular RNA and/or linear mRNA) may be completely or partially located in the interior space of the transfer vehicle, within a bilayer membrane of the transfer vehicle, or associated with the exterior surface of the transfer vehicle.

In some embodiments, a transfer vehicle encapsulates circular RNA. In some embodiments, the transfer vehicle encapsulates at least one circular RNA construct and comprises an ionizable lipid. In some embodiments, the transfer vehicle encapsulates at least one circular RNA construct and comprises an ionizable lipid and an additional lipid selected from a structural lipid, a helper lipid, and a PEG-modified lipid. In some embodiments, the transfer vehicle encapsulates at least one circular RNA construct and comprises an ionizable lipid, a structural lipid, a helper lipid, and/or a PEG-modified lipid. In some embodiments, a transfer vehicle encapsulates at least one circular RNA construct and comprises an ionizable lipid, a structural lipid, a PEG-modified lipid, and a helper lipid. In some embodiments, the transfer vehicle is a lipid nanoparticle.

Without wishing to be bound by theory, it is thought that transfer vehicles described herein shield encapsulated circular RNA from degradation and provide for effective delivery of circular RNA to target cells in vivo and in vitro.

In certain embodiments, the transfer vehicles are formulated based in part upon their ability to facilitate the transfection (e.g., of a circular RNA) of a target cell. In another embodiment, the transfer vehicles may be selected and/or prepared to optimize delivery of circular RNA to a target cell, tissue or organ. For example, if the target cell is a hepatocyte, the properties of the compositions (e.g., size, charge and/or pH) may be optimized to effectively deliver such composition (e.g., lipid nanoparticles) to the target cell or organ, reduce immune clearance and/or promote retention in the target cell or organ. Alternatively, if the target tissue is the central nervous system, the selection and preparation of the transfer vehicle must consider penetration of, and retention within. the blood brain barrier and/or the use of alternate means of directly delivering such compositions to such target tissue (e.g., via intracerebrovascular administration). In certain embodiments, the transfer vehicles may be combined with agents that facilitate the transfer of encapsulated materials across the blood brain barrier (e.g., agents which disrupt or improve the permeability of the blood brain barrier and thereby enhance the transfer of circular RNA to the target cells). While the transfer vehicles described herein can facilitate introduction of circular RNA into target cells, the addition of polycations (e.g., poly L-lysine and protamine) as a copolymer to one or more of the lipid nanoparticles that comprise the pharmaceutical compositions can in some instances markedly enhance the transfection efficiency of several types of transfer vehicles by 2-28 fold in a number of cell lines both in vitro and in vivo (See, N. J. Caplen, et al., Gene Ther. 1995; 2: 603; S. Li, et al., Gene Ther. 1997; 4, 891.).

Transfer vehicles described herein can allow the encapsulated polynucleotide to reach the target cell or may preferentially allow the encapsulated polynucleotide to reach the target cells or organs on a discriminatory basis. Alternatively, the transfer vehicles may limit the delivery of encapsulated polynucleotides to other non-targeted cells or organs where the presence of the encapsulated polynucleotides may be undesirable or of limited utility.

Loading or encapsulating a polynucleotide, e.g., circular RNA, into a transfer vehicle may serve to protect the polynucleotide from an environment (e.g., serum) which may contain enzymes or chemicals that degrade such polynucleotides and/or systems or receptors that cause the rapid excretion of such polynucleotides. Accordingly, in some embodiments, the compositions described herein are capable of enhancing the stability of the encapsulated polynucleotide(s), particularly with respect to the environments into which such polynucleotides will be exposed.

In certain embodiments, the transfer vehicles described herein are prepared by combining multiple lipid components (e.g., one or more of the compounds disclosed herein) with one or more polymer components.

A lipid nanoparticle may be comprised of additional lipid combinations in various ratios. The selection of ionizable lipids, helper lipids, structural lipids, and/or PEG-modified lipids that make up the lipid nanoparticles, as well as the relative molar ratio of such lipids to each other, is based upon the characteristics of the selected lipid(s), the nature of the intended target cells or tissues and the characteristics of the materials or polynucleotides to be delivered by the lipid nanoparticle. Additional considerations include, for example, the saturation of the alkyl chain, as well as the size, charge, pH, pKa, fusogenicity and toxicity of the selected lipid(s).

The formation of a lipid nanoparticle (LNP) described herein may be accomplished by any methods known in the art. See, e.g., U.S. Pat. Pub. No. US2012/0178702 A1, which is incorporated herein by reference in its entirety. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety). Lipid nanoparticles, formulations, and methods of preparation are described in, e.g., International Pat. Pub. No. WO 2011/127255 or WO 2008/103276, U.S. Pat. Pub. No. US2005/0222064 A1, U.S. Pat. Pub. No. US2013/0156845 A1, International Pat. Pub. No. WO2013/093648 A2, WO2012/024526 A2, U.S. Pat. Pub. No. US2013/0164400 A1, and U.S. Pat. No. 8,492,359, all of which are incorporated herein by reference in their entirety.

In some embodiments, the lipid nanoparticle comprises one or more cationic lipids, ionizable lipids, or poly β-amino esters. In some embodiments, the nanoparticle comprises one or more non-cationic lipids. In some embodiments, the lipid nanoparticle comprises one or more PEG-modified lipids, polyglutamic acid lipids, or hyaluronic acid lipids. In some embodiments, the lipid nanoparticle comprises cholesterol. In some embodiments, the lipid nanoparticle comprises arachidonic acid, leukotriene, or oleic acid. In some embodiments, the lipid nanoparticle comprises a targeting moiety, wherein the targeting moiety mediates receptor-mediated endocytosis selectively into cells of a selected cell population in the absence of cell selection or purification. In some embodiments, the lipid nanoparticle comprises more than one circular RNA construct.

Examples of further suitable lipids include the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

A lipid nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

In one embodiment, the lipid nanoparticles may have a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm. In one embodiment, the lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. Each possibility represents a separate embodiment.

In some embodiments, a nanoparticle (e.g., a lipid nanoparticle) has a mean diameter of 10-500 nm, 20-400 nm, 30-300 nm, or 40-200 nm. In some embodiments, a nanoparticle (e.g., a lipid nanoparticle) has a mean diameter of 50-150 nm, 50-200 nm, 80-100 nm, or 80-200 nm.

In some embodiments, the lipid nanoparticles described herein can have a diameter from below 0.1 μm to up to 1 mm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, LNPs may have a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. Each possibility represents a separate embodiment.

A nanoparticle composition may be relatively homogenous. A polydispersity index may be used to indicate the homogeneity of a nanoparticle composition, e.g., the particle size distribution of the lipid nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.1 1, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20. Each possibility represents a separate embodiment.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −20 mV to about −10 mV, from about −20 mV to about −15 mV from about −20 mV to about +20 mV, from about −20 mV to about +15 mV, from about −20 mV to about +10 mV, from about −20 mV to about +5 mV, from about −20 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV. Each possibility represents a separate embodiment.

The efficiency of encapsulation of a therapeutic agent describes the amount of therapeutic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic agent in a solution containing the lipid nanoparticle composition before and after breaking up the lipid nanoparticle composition with one or more organic solvents or detergents. Fluorescence may be used to measure the amount of free therapeutic agent (e.g., nucleic acids) in a solution. For the lipid nanoparticle compositions described herein, the encapsulation efficiency of a therapeutic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

In some embodiments, the lipid nanoparticle has a polydispersity value of less than 0.4. In some embodiments, the lipid nanoparticle has a net neutral charge at a neutral pH. In some embodiments, the lipid nanoparticle has a mean diameter of 50-200 nm.

The properties of a lipid nanoparticle formulation may be influenced by factors including, but not limited to, the selection of the cationic lipid component, the degree of cationic lipid saturation, the selection of the non-cationic lipid component, the degree of noncationic lipid saturation, the selection of the structural lipid component, the nature of the PEGylation, ratio of all components and biophysical parameters such as size. As described herein, the purity of a PEG lipid component is also important to an LNP's properties and performance.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 120 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 50-120 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%. As a non-limiting example, the mol-% of the ionizable lipid may be from about 30 mol-% to about 40 mol-%. As a non-limiting example, the mol-% of the ionizable lipid may be from about 25 mol-% to about 35 mol-%. In some embodiments, the mol-% of the ionizable lipid is about 10 mol-%. In some embodiments, the mol-% of the ionizable lipid is about 15 mol-%. In some embodiments, the mol-% of the ionizable lipid is about 20 mol-%. In some embodiments, the mol-% of the ionizable lipid is about 25 mol-%. In some embodiments, the mol-% of the ionizable lipid is about 30 mol-%. In some embodiments, the mol-% of the ionizable lipid is about 33 mol-%. In some embodiments, the mol-% of the ionizable lipid is about 35 mol-%. In some embodiments, the mol-% of the ionizable lipid is about 40 mol-%. In some embodiments, the mol-% of the ionizable lipid is about 45 mol-%. In some embodiments, the mol-% of the ionizable lipid is about 55 mol-%. In some embodiments, the mol-% of the ionizable lipid is about 60 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%. In some embodiments, the mol-% of the phospholipid is from about 30 mol-% to about 60 mol-%. In some embodiments, the mol-% of the phospholipid is from about 35 mol-% to about 55 mol-%. In some embodiments, the mol-% of the phospholipid is from about 35 mol-% to about 45 mol-%. In some embodiments, the mol-% of the phospholipid is about 10 mol-%. In some embodiments, the mol-% of the phospholipid is about 15 mol-%. In some embodiments, the mol-% of the phospholipid is about 20 mol-%. In some embodiments, the mol-% of the phospholipid is about 25 mol-%. In some embodiments, the mol-% of the phospholipid is about 30 mol-%. In some embodiments, the mol-% of the phospholipid is about 35 mol-%. In some embodiments, the mol-% of the phospholipid is about 40 mol-%. In some embodiments, the mol-% of the phospholipid is about 45 mol-%. In some embodiments, the mol-% of the phospholipid is about 55 mol-%. In some embodiments, the mol-% of the phospholipid is about 60 mol-%.

In some embodiments, the mol-% of the phospholipid as described above comprises two or more phospholipids at an individual mol-% that totals to an aforementioned amount. In certain embodiments, the mol-% of the phospholipid is about 20 mol-% each of two phospholipids. In certain embodiments, the mol-% of the phospholipid is about 15 mol-% each of two phospholipids. In certain embodiments, the mol-% of the phospholipid is about 25 mol-% each of two phospholipids. In certain embodiments, the mol-% of the phospholipid is about 30 mol-% each of two phospholipids. In certain embodiments, the mol-% of the phospholipid is about 15 mol-% of a first phospholipid and about 20 mol-% of a second phospholipid. In certain embodiments, the mol-% of the phospholipid is about 30 mol-% of a first phospholipid and about 10 mol-% of a second phospholipid. In certain embodiments, the mol-% of the phospholipid is about 25 mol-% of a first phospholipid and about 10 mol-% of a second phospholipid. In certain embodiments, the mol-% of the phospholipid is about 25 mol-% of a first phospholipid and about 20 mol-% of a second phospholipid. In certain embodiments, the mol-% of the phospholipid is about 15 mol-% of a first phospholipid and about 20 mol-% of a second phospholipid.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%. In some embodiments, the mol-% of the PEG lipid may be about 2.5 mol-%.

In some embodiments, (a) the PEG lipid is PEG2k-DMG or PEG2k-DSPE or a mixture thereof; (b) the structural lipid is cholesterol; and (c) the phospholipid, non-ionizable lipid or zwitterionic lipid is a sphingolipid or DSPC or a mixture thereof.

In some embodiments, the lipid component of the nanoparticle comprises: (a) about 0 mol % to about 10 mol % of PEG lipid; (b) about 0 mol % to about 30 mol % structural lipid; (c) about 20 mol % to about 45 mol % phospholipid, non-ionizable lipid or zwitterionic lipid; and (d) about 30 mol % to about 60 mol % of a Lipid of the Disclosure.

In some embodiments, the lipid component of the nanoparticle comprises: (a) about 1 mol % to about 2 mol % of PEG lipid; (b) about 25 mol % to about 40 mol % structural lipid; (c) about 20 mol % to about 45 mol % phospholipid, non-ionizable lipid or zwitterionic lipid; and (d) about 30 mol % to about 60 mol % of a Lipid of the Disclosure.

In some embodiments, the lipid component of the nanoparticle comprises: (a) about 2 mol % of PEG lipid; (b) about 25 mol % structural lipid; (c) about 40 mol % phospholipid, non-ionizable lipid or zwitterionic lipid; and (d) about 33 mol % of a Lipid of the Disclosure.

In some embodiments, the lipid component of the nanoparticle comprises: (a) about 2.5 mol % of PEG lipid; (b) about 39 mol % structural lipid; (c) about 10 mol % phospholipid, non-ionizable lipid or zwitterionic lipid; and (d) about 48.5 mol % of a Lipid of the Disclosure.

In some embodiments, the lipid component of the nanoparticle comprises: (a) about 1.5 mol % of PEG lipid; (b) about 40 mol % structural lipid; (c) about 10 mol % phospholipid, non-ionizable lipid or zwitterionic lipid; and (d) about 48.5 mol % of a Lipid of the Disclosure.

In certain embodiments, the lipid component of the nanoparticle composition comprises about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In certain embodiments, the lipid component of the nanoparticle composition comprises about 20 mol % to about 45 mol % ionizable lipid, about 30 mol % to about 60 mol % phospholipid, about 10 mol % to about 30 mol % structural lipid, and about 0 mol % to about 10 mol % of PEG lipid, provided that the total mol % does not exceed 100%. In some embodiments, the lipid component of the nanoparticle composition comprises about 35 mol % to about 55 mol % ionizable lipid, about 5 mol % to about 25 mol % phospholipid, about 30 mol % to about 40 mol % structural lipid, and about 0 mol % to about 10 mol % of PEG lipid, provided that the total mol % does not exceed 100%. In some embodiments, the lipid component of the nanoparticle composition comprises about 30 mol % to about 40 mol % ionizable lipid, about 35 mol % to about 45 mol % phospholipid, about 20 mol % to about 30 mol % structural lipid, and about 0.5 mol % to about 5 mol % of PEG lipid, provided that the total mol % does not exceed 100%. In certain embodiments, the lipid component of the nanoparticle composition comprises about 25 mol % to about 45 mol % ionizable lipid, about 35 mol % to about 50 mol % phospholipid, about 10 mol % to about 25 mol % structural lipid, and about 1 mol % to about 5 mol % of PEG lipid, provided that the total mol % does not exceed 100%. In a particular embodiment, the lipid component comprises about 50 mol % ionizable lipid, about 10 mol % phospholipid, about 38.5 mol % structural lipid, and about 1.5 mol % of PEG lipid. In another particular embodiment, the lipid component comprises about 40 mol % ionizable lipid, about 20 mol % phospholipid, about 38.5 mol % structural lipid, and about 1.5 mol % of PEG lipid. In another particular embodiment, the lipid component comprises about 48.5 mol % ionizable lipid, about 10 mol % phospholipid, about 40 mol % structural lipid, and about 1.5 mol % of PEG lipid. In another particular embodiment, the lipid component comprises about 48.5 mol % ionizable lipid, about 10 mol % phospholipid, about 39 mol % structural lipid, and about 2.5 mol % of PEG lipid. In another particular embodiment, the lipid component comprises about 33 mol % ionizable lipid, about 40 mol % phospholipid, about 25 mol % structural lipid, and about 2 mol % of PEG lipid. In some embodiments, the phospholipid is DOPE or DSPC. In some embodiments, the phospholipid is DSPC. In some embodiments, the phospholipid is a sphingolipid. In some embodiments, the phospholipid is a sphingomyelin. In other embodiments, the PEG lipid is PEG-DMG (eg. PEG2K-DMG). In other embodiments, the PEG lipid is PEG-DSPE (eg. PEG2K-DSPE). In other embodiments, the PEG lipid is PEG-DMPE (eg. PEG2K-DMPE). In other embodiments, the structural lipid is cholesterol. In other embodiments, the PEG lipid is PEG-DMG and/or the structural lipid is cholesterol. In some embodiments, the PEG lipids is PEG2K-DMG, the structural lipid is cholesterol, and the phospholipid is DSPC. In some embodiments, the PEG lipids is PEG2K-DMG, the structural lipid is cholesterol, and the phospholipid is sphingomyelin. In some embodiments, the PEG lipids is PEG-DMG, the structural lipid is cholesterol, and the phospholipid is a mixture of DSPC and sphingomyelin. In certain embodiments, the LNP comprises about 33 mol % ionizable lipid (eg. at least one ionizable lipid of a Formula described herein), about 40 mol % of a sphingolipid, about 25 mol % cholesterol and about 2 mol % PEG2K-DMG. In some embodiments, the PEG lipids is PEG2K-DSPE, the structural lipid is cholesterol, and the phospholipid is DSPC. In some embodiments, the PEG lipids is PEG2K-DSPE, the structural lipid is cholesterol, and the phospholipid is sphingomyelin. In some embodiments, the PEG lipids is PEG-DSPE, the structural lipid is cholesterol, and the phospholipid is a mixture of DSPC and sphingomyelin. In some embodiments, the PEG lipids is PEG2K-DMG, the structural lipid is cholesterol, and the phospholipid is DOPE. In some embodiments, the PEG lipids is PEG2K-DMG, the structural lipid is cholesterol, and the phospholipid is DOPC. In some embodiments, the PEG lipids is PEG2K-DMG, the structural lipid is cholesterol, and the phospholipid is DLPC. In some embodiments, the PEG lipids is PEG2K-DMG, the structural lipid is cholesterol, and the phospholipid is DOPS. In some embodiments, the PEG lipids is PEG-DMG, the structural lipid is cholesterol, and the phospholipid is a mixture of a phosphatidylcholine lipid and a sphingolipid. In some embodiments, the PEG lipids is PEG-DMG, the structural lipid is cholesterol, and the phospholipid is a mixture of a phosphatidylcholine lipid and phosphatidylserine lipid. In some embodiments, the PEG lipids is PEG-DMG, the structural lipid is cholesterol, and the phospholipid is a mixture of a phosphatidylcholine lipid and a phosphoethanolamine lipid. In some embodiments, the PEG lipids is PEG-DMG, the structural lipid is cholesterol, and the phospholipid is a mixture of a sphingolipid and phosphatidylserine lipid. In some embodiments, the PEG lipids is PEG-DMG, the structural lipid is cholesterol, and the phospholipid is a mixture of a sphingolipid and a phosphoethanolamine lipid. In certain embodiments, the LNP comprises about 33 mol % ionizable lipid, about 20 mol % of a sphingolipid, about 20 mol % of a non-sphingolipid phospholipid, about 25 mol % cholesterol and about 2 mol % of a PEGylated lipid. In certain embodiments, the LNP comprises about 33 mol % ionizable lipid, about 10 mol % of a sphingolipid, about 30 mol % of a non-sphingolipid phospholipid, about 25 mol % cholesterol and about 2 mol % of a PEGylated lipid. In certain embodiments, the LNP comprises about 33 mol % ionizable lipid, about 30 mol % of a sphingolipid, about 10 mol % of a non-sphingolipid phospholipid, about 25 mol % cholesterol and about 2 mol % of a PEGylated lipid. In certain embodiments, the LNP comprises about 33 mol % ionizable lipid, about 20 mol % sphingomyelin, about 20 mol % of a DSPC, about 25 mol % cholesterol and about 2 mol % of a PEGylated lipid. In certain embodiments, the LNP comprises about 33 mol % ionizable lipid, about 10 mol % sphingomyelin, about 30 mol % of a DSPC, about 25 mol % cholesterol and about 2 mol % of a PEGylated lipid. In certain embodiments, the LNP comprises about 33 mol % ionizable lipid, about 30 mol % sphingomyelin, about 10 mol % of a DSPC, about 25 mol % cholesterol and about 2 mol % of a PEGylated lipid. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids, wherein each of the phosphatidylcholine, phosphatidylserine, phosphoethanolamine, and sphingoid lipids is present in an amount less than 30 mol % of the total lipid component of the LNP. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids, wherein each of the phosphatidylcholine, phosphatidylserine, phosphoethanolamine, and sphingoid lipids is present in an amount less than 25 mol % of the total lipid component of the LNP. In certain embodiments, LNP is any one of the aforementioned in this paragraph wherein the PEG lipid is PEG2k-DMG. In certain embodiments, LNP is any one of the aforementioned in this paragraph wherein the PEG lipid is PEG2k-DSPE.

In another particular embodiment, LNP comprises about 33 mol % ionizable lipid, about 40 mol % DSPC, about 25 mol % cholesterol, and about 2 mol % of PEG lipid. In another particular embodiment, LNP comprises about 33 mol % ionizable lipid, about 40 mol % sphingomyelin, about 25 mol % cholesterol, and about 2 mol % of PEG lipid. In another particular embodiment, LNP comprises about 33 mol % ionizable lipid, about 40 mol % DOPE, about 25 mol % cholesterol, and about 2 mol % of PEG lipid. In another particular embodiment, LNP comprises about 33 mol % ionizable lipid, about 40 mol % DOPC, about 25 mol % cholesterol, and about 2 mol % of PEG lipid. In another particular embodiment, LNP comprises about 33 mol % ionizable lipid, about 40 mol % DLPC, about 25 mol % cholesterol, and about 2 mol % of PEG lipid. In another particular embodiment, LNP comprises about 33 mol % ionizable lipid, about 40 mol % DOPS, about 25 mol % cholesterol, and about 2 mol % of PEG lipid. In another particular embodiment, LNP comprises about 33 mol % ionizable lipid, about 40 mol % phospholipid, about 25 mol % cholesterol, and about 2 mol % of PEG lipid. In another particular embodiment, LNP comprises about 33 mol % ionizable lipid, about 20 mol % sphingomyelin, about 20 mol % DSPC, about 25 mol % cholesterol, and about 2 mol % of PEG lipid. In certain embodiments, LNP is any one of the aforementioned in this paragraph wherein the PEG lipid is PEG2k-DMG. In certain embodiments, LNP is any one of the aforementioned in this paragraph wherein the PEG lipid is PEG2k-DSPE.

In certain embodiments, the LNP comprises about 43 mol % ionizable lipid, about 15 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 25 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 15 mol % of a sphingolipid, about 25 mol % of a non-sphingolipid phospholipid, about 25 mol % cholesterol and about 2 mol % of a PEGylated lipid. In some embodiments, the PEG lipid is PEG2K-DSPE, the structural lipid is cholesterol, and the phospholipid is a mixture of DSPC and sphingomyelin. In some embodiments, the PEG lipid is PEG2K-DMG, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DSPE. In certain embodiments, the LNP comprises about 48.5 mol % ionizable lipid, about 10 mol % of a phospholipid (such as DSPC), about 40 mol % cholesterol and about 1.5 mol % PEG2K-DMG. In certain embodiments, the LNP comprises about 48.5 mol % ionizable lipid, about 10 mol % of a phospholipid (such as DSPC), about 39 mol % cholesterol and about 2.5 mol % PEG2K-DSPE.

In another particular embodiment, the lipid component includes about 48.5 mol % ionizable lipid, about 10 mol % phospholipid, about 38.5 mol % structural lipid, and about 3 mol % of PEG lipid. In another particular embodiment, the lipid component includes about 48.5 mol % ionizable lipid, about 10 mol % phospholipid, about 38 mol % structural lipid, and about 3.5 mol % of PEG lipid. In some embodiments, the PEG lipid is PEG2K-DPPE, the structural lipid is cholesterol, and the phospholipid is a DSPC or a mixture of DSPC and sphingomyelin. In some embodiments, the PEG lipid is PEG2K-DPPE, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DPPE. In certain embodiments, the LNP comprises about 48.5 mol % ionizable lipid, about 10 mol % of a phospholipid (such as DSPC), about 39.5 mol % cholesterol and about 2 mol % PEG2K-DPPE. In certain embodiments, the LNP comprises about 48.5 mol % ionizable lipid, about 10 mol % of a phospholipid (such as DSPC), about 39 mol % cholesterol and about 2.5 mol % PEG2K-DPPE. In certain embodiments, the LNP comprises about 48.5 mol % ionizable lipid, about 10 mol % of a phospholipid (such as DSPC), about 38.5 mol % cholesterol and about 3 mol % PEG2K-DPPE. In certain embodiments, the LNP comprises about 48.5 mol % ionizable lipid, about 10 mol % of a phospholipid (such as DSPC), about 38 mol % cholesterol and about 3.5 mol % PEG2K-DPPE. In some embodiments, the PEG lipid is PEG2K-DMG, the structural lipid is cholesterol, and the phospholipid is a DSPC or a mixture of DSPC and sphingomyelin. In some embodiments, the PEG lipid is PEG2K-DMG, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DMG. In certain embodiments, the LNP comprises about 48.5 mol % ionizable lipid, about 10 mol % of a phospholipid (such as DSPC), about 39.5 mol % cholesterol and about 2 mol % PEG2K-DMG. In certain embodiments, the LNP comprises about 48.5 mol % ionizable lipid, about 10 mol % of a phospholipid (such as DSPC), about 39 mol % cholesterol and about 2.5 mol % PEG2K-DMG. In certain embodiments, the LNP comprises about 48.5 mol % ionizable lipid, about 10 mol % of a phospholipid (such as DSPC), about 38.5 mol % cholesterol and about 3 mol % PEG2K-DMG. In certain embodiments, the LNP comprises about 48.5 mol % ionizable lipid, about 10 mol % of a phospholipid (such as DSPC), about 38 mol % cholesterol and about 3.5 mol % PEG2K-DMG. In some embodiments, the PEG lipid is PEG2K-DSPE, the structural lipid is cholesterol, and the phospholipid is a DSPC or a mixture of DSPC and sphingomyelin. In some embodiments, the PEG lipid is PEG2K-DSPE, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DSPE. In certain embodiments, the LNP comprises about 48.5 mol % ionizable lipid, about 10 mol % of a phospholipid (such as DSPC), about 39.5 mol % cholesterol and about 2 mol % PEG2K-DSPE. In certain embodiments, the LNP comprises about 48.5 mol % ionizable lipid, about 10 mol % of a phospholipid (such as DSPC), about 39 mol % cholesterol and about 2.5 mol % PEG2K-DSPE. In certain embodiments, the LNP comprises about 48.5 mol % ionizable lipid, about 10 mol % of a phospholipid (such as DSPC), about 38.5 mol % cholesterol and about 3 mol % PEG2K-DSPE. In certain embodiments, the LNP comprises about 48.5 mol % ionizable lipid, about 10 mol % of a phospholipid (such as DSPC), about 38 mol % cholesterol and about 3.5 mol % PEG2K-DSPE.

In some embodiments, the lipid component of the nanoparticle comprises: (a) about 2 mol % of hydrophilic polymer lipid; (b) about 20 mol % to about 45 mol % structural lipid; (c) about 20 mol % to about 45 mol % non-ionizable lipid (e.g., phospholipid), non-ionizable lipid or zwitterionic lipid; and (d) about 30 mol % to about 40 mol % of an ionizable lipid. In some embodiments, the lipid component of the nanoparticle comprises: (a) about 2 mol % of hydrophilic polymer lipid; (b) about 30 mol % structural lipid; (c) about 30 mol % phospholipid, non-ionizable lipid or zwitterionic lipid; and (d) about 38 mol % of an ionizable lipid. In certain embodiments, the LNP comprises about 38 mol % ionizable lipid, about 30 mol % of a sphingolipid, about 30 mol % cholesterol and about 2 mol % of a hydrophilic polymer lipid. In certain embodiments, the LNP comprises about 38 mol % ionizable lipid, about 30 mol % of a phosphatidylcholine lipid, about 30 mol % cholesterol and about 2 mol % of a hydrophilic polymer lipid. In certain embodiments, the LNP comprises about 38 mol % ionizable lipid, about 30 mol % of egg sphingomyelin, about 30 mol % cholesterol and about 2 mol % of a hydrophilic polymer lipid. In certain embodiments, the LNP comprises about 38 mol % ionizable lipid, about 30 mol % of DSPC, about 30 mol % cholesterol and about 2 mol % of a hydrophilic polymer lipid. In certain embodiments, the LNP comprises about 38 mol % ionizable lipid, about 30 mol % of 16:0 SM, about 30 mol % cholesterol and about 2 mol % of a hydrophilic polymer lipid. In certain embodiments, the LNP comprises about 38 mol % ionizable lipid, about 30 mol % of a phospholipid, about 30 mol % cholesterol and about 2 mol % of DMG-PEG2k.

In certain embodiments, the LNP comprises about 38 mol % AX-6, about 30 mol % of a sphingolipid, about 30 mol % cholesterol and about 2 mol % of a hydrophilic polymer lipid. In certain embodiments, the LNP comprises about 38 mol % AX-6, about 30 mol % of a phosphatidylcholine lipid, about 30 mol % cholesterol and about 2 mol % of a hydrophilic polymer lipid. In certain embodiments, the LNP comprises about 38 mol % AX-6, about 30 mol % of egg sphingomyelin, about 30 mol % cholesterol and about 2 mol % of a hydrophilic polymer lipid. In certain embodiments, the LNP comprises about 38 mol % AX-6, about 30 mol % of DSPC, about 30 mol % cholesterol and about 2 mol % of a hydrophilic polymer lipid. In certain embodiments, the LNP comprises about 38 mol % AX-6, about 30 mol % of 16:0 SM, about 30 mol % cholesterol and about 2 mol % of a hydrophilic polymer lipid. In certain embodiments, the LNP comprises about 38 mol % AX-6, about 30 mol % of a phospholipid, about 30 mol % cholesterol and about 2 mol % of DMG-PEG2k.

In certain embodiments, the phospholipid is 16:0 SM (N-palmitoyl-D-erythro-sphingosylphosphorylcholine).

In some embodiments, the LNP further comprises a targeting moiety. In some embodiments, the targeting moiety is an antibody or a fragment thereof.

The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to payload in a nanoparticle composition is from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a payload in a nanoparticle composition may, for example, be measured using absorption spectroscopy (e.g., ultraviolet-visible spectroscopy).

In some embodiments, a nanoparticle composition of the present disclosure is formulated to provide a specific N:P ratio. The N:P ratio of the composition refers to the molar ratio of nitrogen atoms in one or more lipids to the number of phosphate groups in an RNA active agent (e.g., a linear mRNA or circular RNA payload). In general, a lower N:P ratio is preferred. The one or more enzymes, lipids, and amounts thereof is selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio is from about 2:1 to about 8:1. In other embodiments, the N:P ratio is from about 5:1 to about 8:1. For example, the N:P ratio is about 5.0:1, about 5.5:1, about 5.67:1, about 6.0:1, about 6.5:1, or about 7.0:1.

### F. Methods for Preparing Lipid Nanoparticles (LNP) Formulations

In one embodiment, a lipid nanoparticle formulation may be prepared by the methods described in International Publication Nos. WO2011127255 or WO2008103276, each of which is herein incorporated by reference in their entirety. In some embodiments, lipid nanoparticle formulations may be as described in International Publication No. WO2019131770, which is herein incorporated by reference in its entirety.

In some embodiments, circular RNA is formulated according to a process described in U.S. patent application Ser. No. 15/809,680. In some embodiments, the present disclosure provides a process of encapsulating circular RNA in transfer vehicles comprising the steps of forming lipids into pre-formed transfer vehicles (i.e. formed in the absence of RNA) and then combining the pre-formed transfer vehicles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e. heating the separate components) as heating post-formulation (post-formation of nanoparticles) does not increase the encapsulation efficiency of the RNA in the lipid nanoparticles. In contrast, in some embodiments of the novel processes of the present disclosure, the order of heating of RNA does not appear to affect the RNA encapsulation percentage. In some embodiments, no heating (i.e. maintaining at ambient temperature) of one or more of the solutions comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, Tris, ammonium sulfate, sodium bicarbonate, sodium citrate, sodium acetate, potassium phosphate or sodium phosphate. In some embodiments, suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present disclosure. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present disclosure, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e. 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

Nanoparticles can be made in a 1 fluid stream or with mixing processes such as microfluidics and T-junction mixing of two fluid streams, one of which contains the circular RNA and the other has the lipid components.

In some embodiments, the lipid nanoparticles described herein may be synthesized using methods comprising, for example, microfluidic mixers, microstructure-induced chaotic advection (MICA), a Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany), using a micromixer chip, and/or using technology. Exemplary mixers and methods are known in the art.

Additional lipid nanoparticle formulations and methods of producing are described in detail in WO2021226597 and WO2021113777, which are incorporated herein by reference in their entireties. For example, disclosed in WO2021226597 and WO2021113777 is a method of preparing lipid nanoparticle formulations of ionizable lipids 128 and 129 of Table 8 herein. Ethanol phase contained ionizable Lipid 128 or Lipid 129 from Table 8 herein, DOPE, Cholesterol, and DSPE-PEG 2000 (Avanti Polar Lipids Inc.) at a weight ratio of 16:1:4:1 or 62:4:33:1 molar ratio combined with an aqueous phase containing circular RNA and 25 mM sodium acetate buffer at pH 5.2. A 3:1 aqueous to ethanol mixing ratio was used. The formulated LNPs were then dialyzed in 1 L of water and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 g/mL LNPs in PBS (pH 7.4) was measured for Z-average using the Malvern Panalytical Zetasizer Pro. The Z-average and polydispersity index were recorded

### G. Other Delivery Vehicles

In certain embodiments, other delivery vehicles that are known in the art may be used to transport the circular RNA (i.e., are transfer vehicles encompassed herein).

In some embodiments, liposomes or other lipid bilayer vesicles may be used as a component or as the whole transfer vehicle to facilitate or enhance the delivery and release of circular RAN to one or more target cells. Liposomes are usually characterized by having an interior space sequestered from an outer medium by a membrane of one or more bilayers forming a microscopic sack or vesicle. Bilayer membranes of liposomes are typically formed by lipids, i.e. amphiphilic molecules of synthetic or natural origin that comprise spatially separated hydrophobic or hydrophilic domains (Lasic, D, and Papahadjopoulos, D., eds. Medical Applications of Liposomes. Elsevier, Amsterdam, 1998).

In certain embodiments, the transfer vehicle for transporting the circular RNA comprises a dendrimer. Use of “dendrimer” describes the architectural motif of the transfer vehicle. In some embodiments, the dendrimer includes but is not limited to containing an interior core and one or more layers (i.e. generations) that extend or attach out from the interior core. In some of the embodiments, the generations may contain one or more branching points and an exterior surface of terminal groups that attach to the outermost generation. The branching points, in certain embodiments, may be mostly monodispersed and contain symmetric branching units built around the interior core. In some embodiments, the interior core. Synthesis of the dendrimer may comprise the divergent method, convergent growth, hypercore and branched monomer growth, double exponential growth, lego chemistry, click chemistry and other methods as available in the art (Mendes L. et al., Molecules. 2017. 22 (9): 1401 further describes these methods).

In certain embodiments, as described herein, the transfer vehicle for the circular RNA construct comprises a polymer nanoparticle. In some embodiments, the polymer nanoparticle includes nanocapsules and nanospheres. Nanocapsules, in some embodiments, are composed of an oily core surrounded by a polymeric shell. In some embodiments, the circular RNA is contained within the core and the polymeric shell controls the release of the circular RNA. On the other hand, nanospheres comprise a continuous polymeric network in which the circular RNA is retained or absorbed onto the surface. In some embodiments, cationic polymers are used to encapsulate the circular RNA due to the favorable electrostatic interaction of the cations to the negatively charged nucleic acids and cell membrane. The polymer nanoparticle may be prepared by various methods. In some embodiments, the polymer nanoparticle may be prepared by nanoprecipitation, emulsion techniques, solvent evaporation, solvent diffusion, reverse salting-out or other methods available in the art.

In certain embodiments, as described herein, the transfer vehicle for the circular RNA construct comprises a polymer-lipid hybrid nanoparticle (LPHNP). In some embodiments, the LPHNP comprises a polymer core enveloped within a lipid bilayer. In some embodiments, the polymer core encapsulates the circular RNA construct. In some embodiments, the LPHNP further comprises an outer lipid bilayer. In certain embodiments this outer lipid bilayer comprises a PEG-lipid, helper lipid, cholesterol or other molecule as known in the art to help with stability in a lipid-based nanoparticle. The lipid bilayer closest to the polymer core mitigates the loss of the entrapped circular RNA during LPHNP formation and protects from degradation of the polymer core by preventing diffusion of water from outside of the transfer vehicle into the polymer core (Mukherjee et al., In J. Nanomedicine. 2019; 14: 1937-1952).

In certain embodiments, the circular RNA can be transported using a peptide-based delivery mechanism. In some embodiments, the peptide-based delivery mechanism comprises a lipoprotein. Based on the size of the drug to be delivered, the lipoprotein may be either a low-density (LDL) or high-density lipoprotein (HDL). As seen in U.S. Pat. No. 8,734,853B2, high-density lipoproteins are capable of transporting a nucleic acid in vivo and in vitro. In particular embodiments, the lipid component includes cholesterol. In more particular embodiments, the lipid component includes a combination of cholesterol and cholesterol oleate.

In certain embodiments, the circular RNA construct can be transported using a carbohydrate carrier or a sugar-nanocapsule. In certain embodiments, the carbohydrate carrier comprises a sugar-decorated nanoparticle, peptide- and saccharide-conjugated dendrimer, nanoparticles based on polysaccharides, and other carbohydrate-based carriers available in the art. As described herein, the incorporation of carbohydrate molecules may be through synthetic means. In some embodiments, the carbohydrate carrier comprises polysaccharides. These polysaccharides may be made from the microbial cell wall of the target cell. For example, carbohydrate carriers comprised of mannan carbohydrates have been shown to successfully deliver mRNA (Son et al., Nano Lett. 2020. 20(3): 1499-1509).

In certain embodiments, as provided herein, the transfer vehicle for the circular RNA is a glyconanoparticle (GlycoNP). As known in the art, glyconanoparticles comprise a core comprising gold, iron oxide, semiconductor nanoparticles or a combination thereof. In some embodiments, the glyconanoparticle is functionalized using carbohydrates. In certain embodiments, the glyconanoparticle comprises a carbon nanotube or graphene. In one embodiment the glyconanoparticle comprises a polysaccharide-based GlycoNP (e.g., chitosan-based GlycoNP). In certain embodiments, the glyconanoparticle is a glycodendrimer.

In certain embodiments, as provided herein, the circular RNA is transferred through use of an exosome, a type of extracellular vesicle. Exosomes naturally are secreted by various types of cells and are used as a transport vesicle for various forms of cargo. During delivery exosomes can contain and protect specific mRNAs, regulatory microRNAs, lipids, and proteins (Luan et al., Acta Pharmacologica Sinica. 2017. 38:754-763). Naturally, exosomes may be 30 nm to 125 nm.

In some embodiments, the exosome may be made in part from an immune cell. As shown in Haney et al, use of immune cell derived exosomes are able to avoid mononuclear phagocytes (J Control Release. 2015. 207:18-30). In some embodiments, the exosome may be a dendritic cell, macrophage, T-cell, B-cell or derived from another immune cell. As seen in WO/2021/041473A1, various forms of RNAs of varying lengths may be transported through exosome delivery including messenger RNA (mRNA), microRNA (miRNA), long intergenic non-coding RNA (lincRNA), long non-coding RNA (lncRNA), non-coding RNA (ncRNA), non-messenger RNA (nmRNA), small RNA (sRNA), small non-messenger RNA (smnRNA), DNA damage response RNA (DD RNA), extracellular RNA (exRNA), small nuclear RNA (snRNA), small nucleolar RNA (snoRNA), and precursor messenger RNA (pre-mRNA).

In other embodiments, the transfer vehicle may comprise in whole or in part from a fusome. In some embodiments, the fusome is derived from an endoplasmic reticulum of a germline cyst. In certain embodiments, the germline cyst is from a Drosophila ovary.

In certain embodiments, the circular RNA construct may be transported using noncellular and instead be through mechanical delivery mechanisms. In some embodiments, this delivery method includes microneedles, electroporation, continuous pumps and/or gene guns.

In some embodiments, the transfer vehicle of the circular RNA construct is a solution or diluent comprising of a salt or a buffer.

### H. Targeting

In some embodiments, the compositions use targeting moieties that may be bound (either covalently or non-covalently) to the transfer vehicles to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes).

As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. No. 08/835,281, and 60/083,294, which are incorporated herein by reference) and other features useful for in vivo and/or intracellular delivery. In other some embodiments, the compositions demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, in some embodiments, compositions may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions to parenchymal hepatocytes, or alternatively the use of mannose containing sugar residues as a targeting ligand would be expected to direct the compositions to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions in target cells and tissues. Examples of suitable targeting moieties include one or more peptides, proteins, aptamers, vitamins and oligonucleotides.

In particular embodiments, a transfer vehicle comprises a targeting moiety. In some embodiments, the targeting moiety mediates receptor-mediated endocytosis selectively into a specific population of cells or tissue. In some embodiments, the targeting moiety is capable of binding to a T cell antigen. In some embodiments, the targeting moiety is capable of binding to a NK, NKT, or macrophage antigen. In some embodiments, the targeting moiety is capable of binding to a protein selected from the group CD3, CD4, CD8, PD-1, 4-1BB, and CD2. In some embodiments, the targeting moiety is a single chain Fv (scFv) fragment, nanobody, peptide, peptide-based macrocycle, minibody, heavy chain variable region, light chain variable region or fragment thereof. In some embodiments, the targeting moiety is selected from T-cell receptor motif antibodies, T-cell α chain antibodies, T-cell β chain antibodies, T-cell γ chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4R^{a} antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In some embodiments, the targeting moiety is a small molecule binder of an ectoenzyme on lymphocytes. Small molecule binders of ectoenzymes include A2A inhibitors CD73 inhibitors, CD39 or adesines receptors A2aR and A2bR. Potential small molecules include AB928.

In some embodiments, transfer vehicles are formulated and/or targeted as described in Shobaki N, Sato Y, Harashima H. Mixing lipids to manipulate the ionization status of lipid nanoparticles for specific tissue targeting. Int J Nanomedicine. 2018; 13:8395-8410. Published 2018 Dec. 10. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

In some embodiments, the target cells are deficient in a protein or enzyme of interest. In some embodiments, the compositions of the present disclosure transfect the target cells on a discriminatory basis (i.e., do not transfect non-target cells). The compositions of the present disclosure may also be prepared to preferentially target a variety of target cells, which include, but are not limited to, immune cells, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions of the present disclosure may be prepared to preferentially distribute to target cells, including but not limited to the heart, lungs, kidneys, liver, and spleen, ocular, or cells in the central nervous system. In some embodiments, the compositions of the present disclosure distribute into the cells of the liver or spleen to facilitate the delivery and the subsequent expression of the circRNA comprised therein by the cells of the liver (e.g., hepatocytes) or the cells of spleen (e.g., immune cells). The targeted cells may function as a biological “reservoir” or “depot” capable of producing, and systemically excreting a functional protein or enzyme.

In some embodiments, the transfer vehicles comprise circRNA which encode a deficient protein or enzyme. Upon distribution of such compositions to the target tissues and the subsequent transfection of such target cells, the exogenous circRNA loaded into the transfer vehicle (e.g., a lipid nanoparticle) may be translated in vivo to produce a functional protein or enzyme encoded by the exogenously administered circRNA (e.g., a protein or enzyme in which the subject is deficient). Accordingly, the compositions of the present disclosure exploit a subject's ability to translate exogenously- or recombinantly-prepared circRNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

### 6. Pharmaceutical Compositions

In certain embodiments, provided herein are compositions (e.g., pharmaceutical compositions) comprising a therapeutic agent provided herein. In some embodiments, the therapeutic agent is a circular RNA polynucleotide provided herein. In some embodiments, the therapeutic agent is a linear mRNA polynucleotide provided herein. In some embodiments the therapeutic agent is a vector provided herein. In some embodiments, the therapeutic agent is a cell comprising a circular RNA, a precursor polynucleotide, a linear mRNA, or vector provided herein (e.g., a human cell, such as a human T cell).

In certain embodiments, the composition further comprises a pharmaceutically acceptable carrier or excipient. In some embodiments, and as described elsewhere herein, the pharmaceutical composition comprises at least one circular RNA and/or linear mRNA polynucleotide and a transfer vehicle. In some embodiments, the transfer vehicle comprises at least one of (i) an ionizable lipid, (ii) a structural lipid, and (iii) a PEG-modified lipid.

In some embodiments, the transfer vehicle is a nanoparticle or lipid nanoparticle. In some embodiments, the nanoparticle is a lipid nanoparticle (LNP), a core-shell nanoparticle, a biodegradable nanoparticle, a biodegradable lipid nanoparticle, a polymer nanoparticle, a polyplex or a biodegradable polymer nanoparticle.

In some embodiments, the pharmaceutical composition comprises a targeting moiety. The targeting moiety mediates receptor-mediated endocytosis, endosome fusion, or direct fusion into selected cells of a selected cell population or tissue in the absence of cell isolation or purification. In some embodiments, the pharmaceutical composition comprises a targeting moiety operably connected to the nanoparticle. In some embodiments, the targeting moiety is a small molecule, scFv, nanobody or VHH, peptide, cyclic peptide, di or tri cyclic peptide, minibody, polynucleotide aptamer, engineered scaffold protein, heavy chain variable region, light chain variable region, or a fragment thereof. In some embodiments, less than 1%, by weight, of the polynucleotides in the composition are double stranded RNA, DNA splints, DNA template, or triphosphorylated RNA. In some embodiments, less than 1%, by weight, of the polynucleotides and proteins in the pharmaceutical composition are double stranded RNA, DNA splints, DNA template, triphosphorylated RNA, phosphatase proteins, protein ligases, RNA polymerases, and capping enzymes.

In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein. In certain embodiments, the pharmaceutical composition comprises a preservative. In some embodiments, the pharmaceutical composition comprises a buffering agent.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

Formulations for oral, aerosol, parenteral (e.g., subcutaneous, intravenous, intraarterial, intramuscular, intradermal, intraperitoneal, and intrathecal), and topical administration are known in the art. More than one route can be used to administer the therapeutic agents provided herein, and in certain instances, a particular route can provide a more immediate and more effective response than another route.

In certain embodiments, the therapeutic agents provided herein can be formulated as inclusion complexes, such as cyclodextrin inclusion complexes, or LNPs or liposomes.

In some embodiments, the composition comprises a precursor RNA polynucleotide described herein, a polynucleotide described herein, a circular RNA polynucleotide described herein, or combinations thereof; and a transfer vehicle described herein. In some embodiments, the transfer vehicle comprises at least one of (i) an ionizable lipid, (ii) a structural lipid, and (iii) a PEG-modified lipid. In some embodiments, the composition further comprises a targeting moiety.

In some embodiments, the therapeutic agents provided herein are formulated in time-released, delayed release, or sustained release delivery systems such that the delivery of the composition occurs prior to, and with sufficient time to, cause sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the present disclosure are formulated such that they are suitable for extended-release of the circRNA and/or linear mRNA contained therein. Such extended-release compositions may be conveniently administered to a subject at extended dosing intervals. For example, in one embodiment, the compositions of the present disclosure are administered to a subject twice a day, daily or every other day. In an embodiment, the compositions of the present disclosure are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by a polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments, the polypeptide is expressed for a longer period of time when encoded by circular RNA than by linear mRNA. For example, the polypeptide may be detected for hours or days longer than the half-life observed with mRNA encoding the polypeptide. In some embodiments the expression of the polypeptide is sustained at least at a therapeutic level. In some embodiments, the polypeptide is expressed at least at a therapeutic level for more than one, more than four, more than six, more than 12, more than 24, more than 48, or more than 72 hours after administration. In some embodiments, the polypeptide is detectable at a therapeutic level in patient tissue (e.g., liver or lung). In some embodiments, the level of detectable polypeptide is from continuous expression from the circRNA composition over periods of time of more than one, more than four, more than six, more than 12, more than 24, more than 48, or more than 72 hours after administration. For example, in some embodiments, where the circular RNA provided herein encodes a CAR (e.g., CD19 CAR), the CAR exhibits greater duration of expression (e.g., in immune cells) and/or functionality (e.g., depletions of B cells) over a longer period of time as compared to a linear mRNA polynucleotide comprising the same expression sequence.

In certain embodiments, a protein encoded by a polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the polypeptide upon other therapeutic intervention, e.g., upon direct injection of the corresponding polypeptide, at one or more comparable time points.

In certain embodiments, the levels of a protein encoded by a polynucleotide are detectable at 3 days, 4 days, 5 days, or 1 week or more after administration. Increased levels of protein may be observed in a tissue (e.g., liver or lung).

In some embodiments, the method yields a sustained circulation half-life of a protein encoded by a polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

In some embodiments, the modified polynucleotide described herein is a circular RNA that affects net charge, and therefore may be suitable for use with a delivery or transfer vehicle comprising an ionizable lipid.

Different types of release delivery systems are available and known to those of ordinary skill in the art. See, e.g., U.S. Pat. Nos. 5,075,109, 4,452,775, 4,667,014, 4,748,034, and 5,239,660, 3,832,253 and 3,854,480. In some embodiments, the therapeutic agent can be conjugated either directly or indirectly through a linking moiety to a targeting moiety. Methods for conjugating therapeutic agents to targeting moieties is known in the art. See, e.g., Wadwa et al., J, Drug Targeting 3:111 (1995) and U.S. Pat. No. 5,087,616. In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150).

In certain embodiments, the compositions may be loaded with diagnostic radionuclide, fluorescent materials or other materials that are detectable in both in vitro and in vivo applications. For example, suitable diagnostic materials may include Rhodamine-dioleoylphosphatidylethanolamine (Rh-PE), Green Fluorescent Protein circRNA (GFP circRNA), Renilla Luciferase circRNA and Firefly Luciferase circRNA.

### 7. Therapeutic Methods

Provided herein are methods of treating a subject in need thereof comprising administering a therapeutically effective amount of the circular RNA and/or linear mRNA provided herein and/or a composition comprising the circular RNA and/or linear mRNA provided herein. Provided herein are also methods of preventing a disease or disorder in a subject in need thereof comprising a therapeutically effective amount of circular RNA and/or linear mRNA provided herein and/or a composition comprising the circular RNA and/or linear mRNA provided herein. In some embodiments, in addition to the circular RNA and/or linear mRNA, a delivery vehicle, and optionally, a targeting moiety operably connected to the delivery vehicle is administered.

In certain aspects, provided herein is a method of producing a protein of interest in a subject in need thereof by introducing or administering a pharmaceutical composition comprising a circular RNA and/or linear mRNA, described herein.

In certain embodiments, the therapeutic agents provided herein (e.g., circular RNA and/or linear mRNA and/or composition comprising the circular RNA and/or linear mRNA) are co-administered with one or more additional therapeutic agents (e.g., in the same pharmaceutical composition or in separate pharmaceutical compositions). In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

In some embodiments, provided herein is a method of treating an autoimmune disorder in a subject by introducing or administering the circular RNA and/or linear mRNA construct provided herein and/or a composition comprising the circular RNA and/or linear mRNA construct provided herein.

In some embodiments, provided herein is a method of treating cancer in a subject by introducing or administering the circular RNA and/or linear mRNA construct provided herein and/or a composition comprising the circular RNA and/or linear mRNA construct provided herein.

In some embodiments, the circular RNA and/or linear mRNA construct encodes a CD19 CAR having the ability to recognize CD19, such that the CD19 CAR, when expressed by a cell, is able to mediate an immune response against the cell expressing the CD19 antigen for which the CAR is specific. Thus, in certain embodiments, provided herein are methods of treating and/or preventing a disease in a subject (e.g., mammalian subject, such as a human subject). In this regard, an embodiment provided herein provides a method of treating or preventing a disease, e.g., cancer, e.g., multiple myeloma, in a subject, comprising administering to the subject the circular RNA and/or linear mRNA therapeutic agents, and/or the pharmaceutical compositions provided herein in an amount effective to treat or prevent the disease in the subject.

In some embodiments, the subject has an autoimmune disease or disorder.

In some embodiments, the subject has a cancer selected from the group consisting of: acute myeloid leukemia (AML); alveolar rhabdomyosarcoma; B cell malignancies; bladder cancer (e.g., bladder carcinoma); bone cancer; brain cancer (e.g., medulloblastoma and glioblastoma multiforme); breast cancer; cancer of the anus, anal canal, or anorectum; cancer of the eye; cancer of the intrahepatic bile duct; cancer of the joints; cancer of the neck; gallbladder cancer; cancer of the pleura; cancer of the nose, nasal cavity, or middle ear; cancer of the oral cavity; cancer of the vulva; chronic lymphocytic leukemia; chronic myeloid cancer; colon cancer; esophageal cancer, cervical cancer; fibrosarcoma; gastrointestinal carcinoid tumor; head and neck cancer (e.g., head and neck squamous cell carcinoma); Hodgkin lymphoma; hypopharynx cancer; kidney cancer; larynx cancer; leukemia; liquid tumors; lipoma; liver cancer; lung cancer (e.g., non-small cell lung carcinoma, lung adenocarcinoma, and small cell lung carcinoma); lymphoma; mesothelioma; mastocytoma; melanoma; multiple myeloma; nasopharynx cancer; non-Hodgkin lymphoma; B-chronic lymphocytic leukemia; hairy cell leukemia; Burkitt's lymphoma; ovarian cancer; pancreatic cancer; cancer of the peritoneum; cancer of the omentum; mesentery cancer; pharynx cancer; prostate cancer; rectal cancer; renal cancer; skin cancer; small intestine cancer; soft tissue cancer; solid tumors; synovial sarcoma; gastric cancer; teratoma; testicular cancer; thyroid cancer; and ureter cancer.

In some embodiments, the subject has an autoimmune disorder selected from scleroderma, Grave's disease, Crohn's disease, Sjogren's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, thyroiditis, and the generalized autoimmune diseases typified by human Lupus.

In the field of ex vivo CAR-T cell therapeutics, “T cell expansion is limited by low rates, and T-cell products of limited functionality” (see, e.g., Cheung et al., Nature Biotechnology, 2018) and the efficacy “is limited by the poor proliferation and persistence of the engineered T cells” (see, e.g., Zhang et al., Nature Biomedical Engineering, 2024) (the contents of both of which are hereby incorporated by reference). In some embodiments, the method of treating the subject comprises administering a bioscaffold in addition to administering the circular RNA and/or linear mRNA encoding the CD19 CAR or composition thereof.

In some embodiments, the bioscaffold is an injectable biomaterial. In some embodiments, the bioscaffold is a biomaterial-derived injectable. In some embodiments, the bioscaffold comprises or is derived from mesoporous silica rods. In some embodiments, the bioscaffold is loaded with interleukin-2. In some embodiments, the bioscaffold comprises a lipid bilayer, e.g., prepared with anti-CD3 and anti-CD28. Bioscaffolds are known and have been generated in the art. See, e.g., Kim et al., Nature Biotechnology, 2014; Cheung 2018 supra; Li et al., Nature Materials, 2018; Dellacherie et al., Advanced Functional Materials, 2020; Zhang et al., Nature Protocols, 2020; Zhang 2024 supra (the contents of each of which are hereby incorporated by reference).

In some embodiments, administering the circular RNA and/or linear mRNA encoding the CD19 CAR in combination with administration of the bioscaffold is capable of ameliorating disease in the subject as compared to control absent bioscaffold administration. In some embodiments, administering the circular RNA and/or linear mRNA encoding the CD19 CAR in combination with administration of the bioscaffold is capable of improving activation, proliferation, stimulation, restimulation, and/or persistence of immune cells (e.g., T cells, NK cells, etc.) in the subject as compared to a control absent bioscaffold administration.

In some embodiments, the bioscaffold is administered to a subject having cancer. In some embodiments, the bioscaffold is injected into the subject. In some embodiments, the bioscaffold is injected subcutaneously into the subject. In some embodiments, the bioscaffold is administered to a subject having a solid tumor. The bioscaffold may be delivered into or adjacent to the solid tumor. In some embodiments, the bioscaffold is injected into or adjacent to the site of an oral cancer. In some embodiments, the bioscaffold is injected into or adjacent to the site of cervical cancer.

In some embodiments, the bioscaffold is administered prior to administration of the circular RNA and/or linear mRNA encoding the CD19 CAR. In some embodiments, the bioscaffold may be administered 1, 2, 3, 4, 5, 6, 7, 8, 9, 10, 11, 12, 13, 14, 15, 16, 17, 18, 20, 21, 22, 23, 24, 25, 26, 27, 28, 29, 30, or more days prior to administration of the circular RNA and/or linear mRNA encoding the CD19 CAR or composition thereof, which may allow for vascularization of the administered bioscaffold material.

In some embodiments, the bioscaffold is administered concurrently with administration of the circular RNA and/or linear mRNA encoding the CD19 CAR or composition thereof. In some embodiments, the bioscaffold is administered after administration of the circular RNA and/or linear mRNA encoding the CD19 CAR or composition thereof.

In certain embodiments, therapeutic methods of the present disclosure further comprise administration of one or more anti-inflammatory agents, e.g., glucocorticoid(s), to a subject. In certain embodiments, the present disclosure provides methods of treating a subject in need thereof comprising administering (i) a therapeutically effective amount of the circular RNA and/or linear mRNA provided herein and/or a composition comprising the circular RNA and/or linear mRNA provided herein and (ii) one or more anti-inflammatory or immunosuppressing agents. In certain embodiments, the administration of the one or more anti-inflammatory or immunosuppressing agents occurs before administration of the pharmaceutical composition comprising the circular RNA and/or linear mRNA. In certain embodiments, the administration of the one or more anti-inflammatory or immunosuppressing agents occurs after administration of the pharmaceutical composition comprising the circular RNA and/or linear mRNA. In certain embodiments, the administration of the one or more anti-inflammatory or immunosuppressing agents occurs simultaneously to the administration of the pharmaceutical composition comprising the circular RNA and/or linear mRNA. In certain embodiments, the one or more anti-inflammatory or immunosuppressing agents is selected from the group consisting of dexamethasone, tocilizumab, kevzara, colcrys, hydroxychloroquine, chloroquine, and a kinase inhibitor. In certain embodiments, the one or more anti-inflammatory agents is dexamethasone or an analog thereof. In certain embodiments, the dexamethasone or an analog thereof is administered before the pharmaceutical composition comprising the circular RNA and/or linear mRNA provided herein.

### EXAMPLES

The disclosure is further described in detail by reference to the following examples but are not intended to be limited to the following examples. These examples encompass any and all variations of the illustrations with the intention of providing those of ordinary skill in the art with complete disclosure and description of how to make and use the subject disclosure and are not intended to limit the scope of what is regarded as the disclosure.

### Example 1: Production of oRNA

### A. Production and Selection of Internal Ribosome Entry Sites (IRES)

### Non-Synthetic IRES Screening and Selection

Naturally occurring and optionally truncated untranslated region (UTR) sequences were selected as internal ribosome entry sites (IRES) candidates. 10,304 IRES sequences were synthesized by a gene synthesis vendor in 11 pools (each pool comprised of approximately 701-999 sequences per pool). IRES sequences received one out of 8 different barcode sequences. To clone the IRES sequences, a plasmid backbone was designed to comprise a 5′ intron element, a 5′ exon element, a Gaussia luciferase expression sequence, a 3′ exon element, and a 3′ intron element along with a 5′primer (e.g., ATGGGAGTG AAGGTGCTGTTCGC (SEQ ID NO: 404)) and a 3′primer (e.g., TTTTTGTTGGTTTGTGTTTGTTTTGTT TGGTGTTTGC (SEQ ID NO: 405)). The 5′and 3′ intron and exon elements of the plasmid backbone were derived from Anabaena. The plasmid backbones were then amplified, and the original backbone attached to the IRES sequences were digested using Dpnl.

Each pool was cloned individually by combining the plasmid backbone and the IRES sequence at a 1:2 ratio, respectively, and incubated with a Gibson Assembly HiFi master mix at 50° C. for 30 minutes. 2 μL of the solution was transformed into 5-alpha competent cells. Transformants were pooled and grown in 1 L LB-ampicillin overnight and plasmid was isolated using Qiagen MaxiPrep kit. Estimated clones per fragment were determined by back calculating from the number of bacterial colonies. FIG. 104 shows the estimated number of clones produced per IRES for the number of sequences contained in each of the 11 pools.

### Synthetic Internal Ribosome Entry Site (IRES) Design and Engineering

2,838 naturally occurring internal ribosome entry sites (IRES) sequences were collected and/or derived from publicly available viral genomic databases. The IRES sequences were inserted into circular RNAs encoding a luciferase reporter protein (e.g., Gaussia Luciferase); the resulting circular RNAs were transfected into primary human hepatocytes (PHH), T cell lymphoma (TCL), and/or myotubes and analyzed for luciferase expression levels as compared to Comparative IRES 1. IRESs were characterized as low expressing based on the fold change calculated as compared to Comparative IRES 1 (i.e., if the circular RNA comprising the tested IRES had a lower or similar value of relative light units (fluorescent activity) as compared to a circular RNA comprising the Comparative IRES 1, it was characterized as low expressing). IRES constructs with a fold change value of less than 1.0 were characterized as low expressing. IRESs were characterized as high expressing based on a fold change value of 1.0 or greater as compared to comparative IRES 1 (i.e., if the circular RNA comprising the tested IRES had a higher value of relative light units as compared to a circular RNA comprising the Comparative IRES 1, it was characterized as high expressing).

For both low and high expressing IRES sequences, sequence structure clustering was performed.

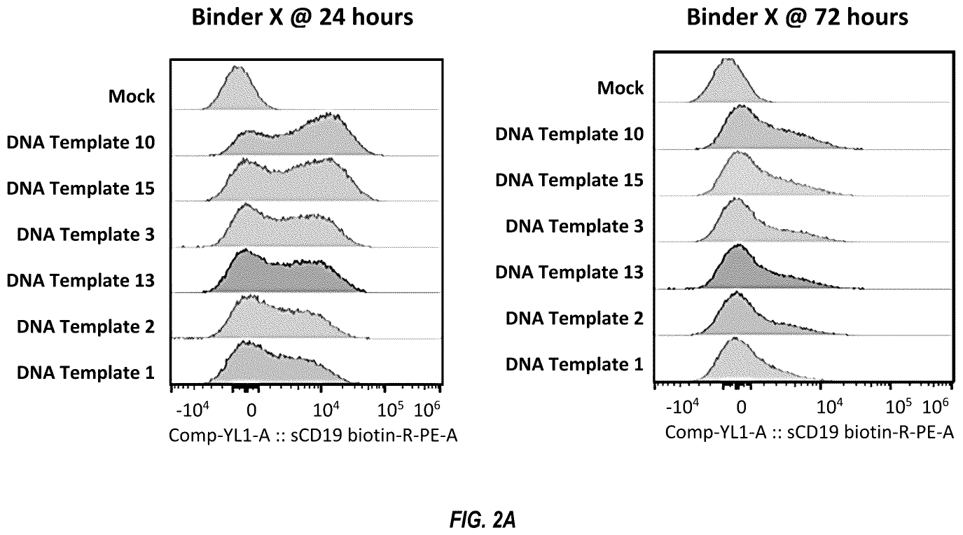

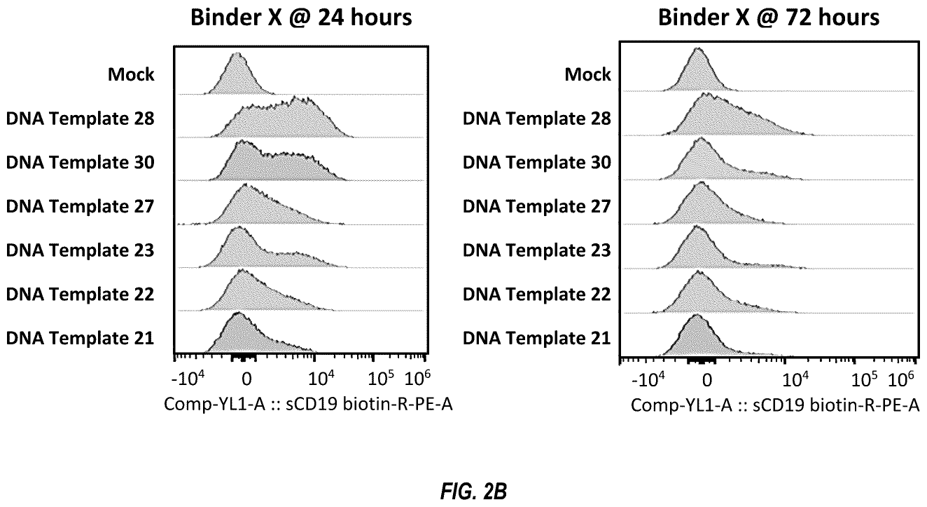

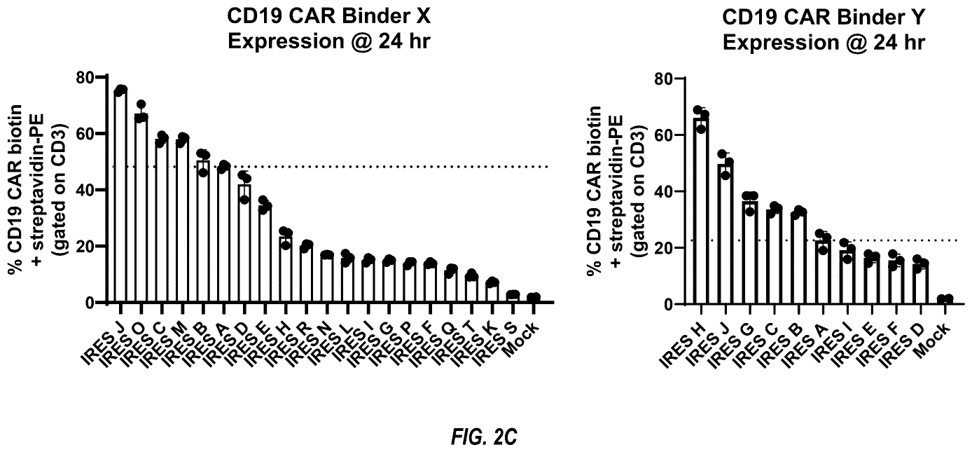

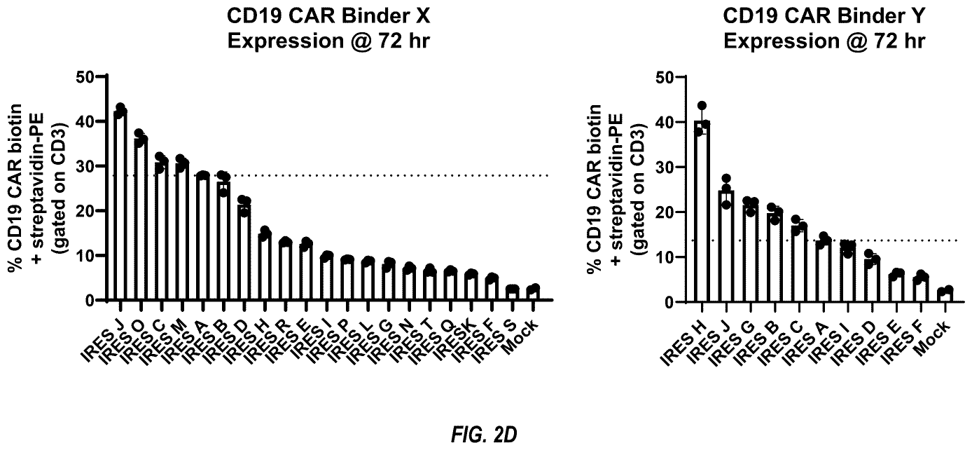

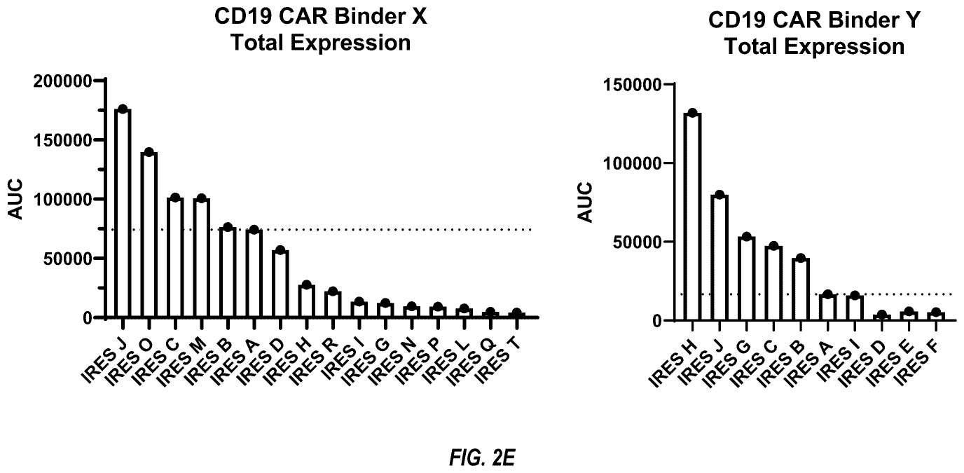

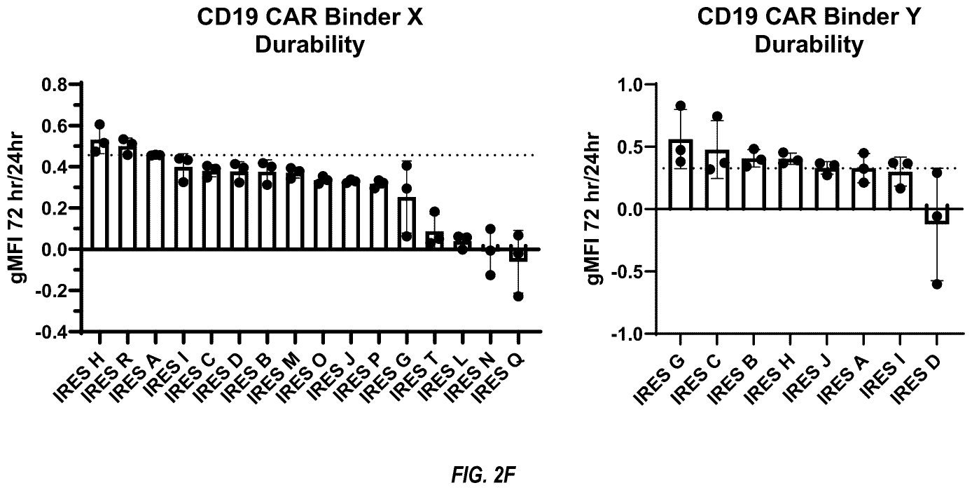

The IRES sequences were analyzed as compared to an exemplary Type I IRES (e.g., CVB3 IRES). Folding constraints were generated for one or more IRES sequences to aid with alignment of the tested IRES to the exemplary IRES. Sequences between domains (e.g., linker spaces) were defined and the domains themselves were folded between the linker spaces in a commercially or publicly available folding software (e.g., RNAFOLD, Vienna). Domains for one or more IRES were identified based on sequence homology to the exemplary IRES (e.g., CVB3 IRES). For example, tested IRES sequences belonging to Type I IRES were analyzed for homology of domains and annotated for similarities and differences in the sequences as compared to an annotated CVB3 IRES (annotated CVB3 IRES is illustrated in FIG. 2). The IRES group I intron sequences were aligned to correspond with one or more domains of the CVB3 IRES.

The naturally occurring IRES was subject to engineering using one or more deletions, substitutions, and/or additions at the primary structure, including the inclusion or deletion of structural motifs present in high expressing naturally occurring IRES to form a synthetic set of IRES. Following the engineering, the synthetic IRES or one or more of its domains was assessed using the folding software, reviewed for its initial folding conformation (i.e., the structure shown by folding software prior to using folding constraints), and given folding constraints and refolded using the folding software (i.e., constrained folded IRES). The initial folding conformation and constrained folded IRES variations were compared to other IRES (e.g., CVB3) to select for and/or generate: a desired shift in subdomain or domain structure (for IRES variations having additions and/or deletions (e.g., for C-loop variants or cryptic codons); locations of predicted favorable truncations; optimal locations for aptamer additions; or other alterations to form variations of synthetic IRES.

### Comparative IRES 1:

CCCCCCTCCCCCCCTTCCCTTCCCTTTGCAACGCAACAATTGTAAGTG
CCCTCACCTGTCAATTGGGACCACCACTTTCAGTGACCCCATGCGAAG
TGCTGAGAGAAAGGAAGCTTTCTTACCCTTCATTTGTGAACCCACTGG
TCTAAGCCGCTTGGAATACGATGAGTGGAAAAGTTCATTCTTAATGGA
GTGAAACATGCTTAAATTTCCAGCTCGTGCTGGTCTTTCCAGTACGGG
GCGGCCCTGTCTGGCCGTAATTCTTCAGAGTGTCACGCCACACTTGTG
GATCTCACGTGCCACATGACAGCGCTACAGCTGGAACTGGGTGCTTGG
TGCCCATGGAGTAACAGCGAAAAGTGTTAGATCAAGCCTTGCTTGGGC
TATGAGCCTGCGGAACAACAACTGGTAACAGTTGCCTCAGGGGCCGAA
AGCCACGGTGTTAACAGCACCCTCATAGTTTGATCCACCTCAGGGTGG
TGATGTTTAGCAGTTAGTAGTTGCCAATCTGTGTTCACTGAAATCTCG
GCATACCGTGTAGTGTACAGGGGTGAAGGATGCCCAGAAGGTACCCGT
AGGTAACCTTAAGAGACTATGGATCTGATCTGGGGCCTTGTCCGGAGT
GCTTTACACACGGCTCAAGGTTAAAAAACGTCTAGCCCCACAGAGCCC
GAGGGATTCGGGTTTTCCCTTTAAAAACCCGACTAGAGCTTATGGTGA
CAATTATTGCTGTTCAGACGAACAGTGTAATTGTTGTCTATTCACAGC
AGTTCTATCAGAGCTTTTCCCACAACGGATCTTCTTGGCAAGCAAATA
CAGCAGGAGTCAAT

### Generating Circular RNAs Comprising a Non-Synthetic or Synthetic IRES

Circular RNA was generated from the pooled plasmid output of the IRES above. The plasmids of the pooled plasmid output were linearized with XbaI, and incubated with NO NTPs, GMP, in vitro transcription (IVT) buffer, RNase Inhibitor, Pyrophosphatase, and T7 polymerase for 2 hours at 37° C. in a thermomixer shaking at 350 RPM. DNaseI was then added to the reaction mix and incubated for 15 minutes at 37° C. Triplicates of resulting IVT product were purified using Monarch RNA purification column to conduct an IRES circularization assay. For enrichment of the circularized product, each replicate of the IVT product was subjected to exonuclease treatment of 15 μg of cleaned IVT product was incubated with RNase R buffer (Fisher Scientific), 10U of RNase R (Fisher Scientific), 1U of XRN-1 (New England Biolabs) for 45 min at 37° C. Circular RNA enriched product was purified using Monarch RNA purification column.

For polysome and stability profiling, the IVT products were then diluted with 2.5× volumes of water with 3.5× volumes of dT resin binding buffer and incubated at 55° C. for 10 minutes. The IVT products in dT resin binding buffer are then bound to 2.5× volumes equilibrated Poros dT resin, and then incubated at 70° C. for 15 minutes at 700 RPM of shaking. Supernatant was then spun through the resin and purified using Monarch RNA purification column using manufacturer specifications.

A circular RNA ladder was generated by cloning 8 scrambled IRES sequences to the same plasmid backbone as the IRES library. For circularization profiling, the IVT products of these plasmids were mixed at defined concentrations across 4 orders of magnitude. The IVT products spiked-in to the IRES IVT product and were subjected to exonuclease treatment with the IRES IVT product. For polysome and stability profiling experiments, the IVT products of these ladders were cleaned up using dT resin and the purification column as described above. The purified products were mixed at defined concentrations across 4 orders of magnitude.

The top IRES sequences were selected based on protein expression (e.g., polysome profiling) and stability measurements (e.g., circular RNA cellular half-life).

### Cytoplasmic Polysome Profiling and Total RNA Isolation of Circular RNA Comprising IRES

### (i) T Cell Electroporation

Human pan-T cells were grown with Advanced RPMI media, supplemented with Normocin, 10% FBS, Penicillin and Streptomycin, and GlutaMAX. T-cells were activated after thawing by adding CD3 and CD34 for 3 days, and then replaced with media containing IL-2. Cells were expanded in this media, keeping density under 3×10⁶/ml, to appropriate cell numbers. Electroporation was performed using the 4D Nucleofector X unit and P3 primary cell 4D Nucleofector X kit L. Cells were resuspended in P3 solution containing 50 μg IRES library/mL at a density of 5×10⁷ cells/mL. 100 μL of the resuspended solution was transferred to a cuvette and electroporated. Cells were then resuspended in 5 mL T cell media per 100 μL electroporated cells (density of 1×10⁶ cells/mL) and placed in a growth container.

b. T-Cell Collection

60M of the resuspended cells were electroporated for polysome experiments, and 30M of the resuspended cells were electroporated for stability experiments. Polysome collection was performed by taking all cells in the growth container at 6 hours post electroporation and adding cycloheximide at 100 μg/ml. These cells were then incubated at 37° C. for 15 minutes and received 2 washes with PBS+cycloheximide at 100 μg/ml per wash. After removal of the final wash (i.e., the second wash), the cells were flash frozen on dry ice and stored at −80° C. The same process was performed for 4 different time points (e.g., 1.5 hours, 6 hours, 24 hours, and 48 hours) with a portion of the cell pool removed to a conical tube and treated with cycloheximide.

c. Polysome Profiling and RNA Isolation

Cell pellets were thawed briefly on ice, and then resuspended in polysome isolation buffer (e.g., 25 mM Tris-HCl pH 7.5, 100 mM KCl, 5 mM MgCl₂, 1% IGEPAL-CA630, 1% Sodium Deoxycholate, 100 μg/ml Cycloheximide, 1 mM DTT, 60 U/ml Superase Inhibitor, 1× complete EDTA free Protease Inhibitor). The solution was then incubated on ice for 30 minutes. After 30 minutes, the debris was spun down at 21,000 xg, 10 minutes, 4° C. For the stability experiment, RNA extraction was directly performed. Trizol LS was added at 4× volume, and 100% Ethanol was added at 5× volume, then RNA extraction was performed using Zymo Direct-zol magbead RNA kit using manufacturer's recommendations.

For polysome experiments, 10-50% sucrose gradients were created using guidelines and standard protocols from a Biocomp Gradient Master instrument. Gradients were layered with the RNA extracts and centrifuged using Beckman XE-90 ultracentrifuge and SW-41Ti rotor at 41,000 RPM for 1.5 hours. These gradients were then fractionated using Biocomp Piston Gradient Fractionator and Gilson Fraction collector, where it collected 250 μL fractions along the gradient.

The circRNA ladder with scrambled IRES sequences was then spiked into each fraction at 2% v/v, and RNA was extracted from each fraction as described in Example 88.

d. Total RNA-Seq Library Generation

For the circularization profiling, IVT and exonuclease treated RNA samples were made to RNA-seq libraries using NEBNext Ultra II Directional RNA-seq library kit and NEBNext Multiplex Oligos for Illumina with Unique Dual Index for RNA following their manuals. Libraries were then pooled in equimolar amounts and sequenced using Illumina Nextseq 2000, P2 kit reagents.

e. circRNA Library Generation

For the stability profiling, total RNA from each timepoint and replicate was spiked in at 0.01% concentration, and measured by RNA concentration, using the scrambled circular RNA ladder described above. For the polysome profiling, all fractions corresponding to RNA bound to 4 or more ribosomes were pooled equally (henceforth “high” library), and all fractions corresponding to RNA bound to less than 4 ribosomes were pooled equally into a separate pool (henceforth “low” library). For each of these polysome pools and stability samples, RNA integrity was measured using Tape Station to validate that the RNA Integrity score was 9 or greater. These RNA samples were then depleted of rRNA using Ribo Minus rRNA depletion kit, using the low input or high input protocol depending on RNA amount. The output was then treated with 5 Units RNase R from Lucigen for 15 minutes at 37° C. The resulting RNA was purified using Beckman RNAclean XP beads. RNA libraries were prepared using NEBNext Ultra II Directional RNA-seq library kit and NEBNext Multiplex Oligos for Illumina with Unique Dual Index for RNA using the pre-enriched protocol. Libraries were then pooled in equimolar amounts and sequenced using Illumina Nextseq 2000, P3 kit reagents.

f. Bioinformatic Analysis of the IRES Library

Each circRNA within the IRES library and scrambled spike in was quantified by a custom RNA-seq protocol. Sequence reads were monitored for quality, and low-quality reads were trimmed using cutadapt. The sequence readings were then mapped to the library reference using BWA, and UMIs. Next, the sequence readings were grouped, called for consensus, and filtered for PCR duplicates and low-quality mappings using fgbio tools. The resultant files were quantified using Kallisto. Kallisto quantities were compiled into a counts table for each of the profiling experiments.

For the circularization experiment, the enrichment of each construct in each replicate was calculated by contrasting the count in the exonuclease treated library to the IVT library after filtering for a minimal count after library size normalization.

For polysome and stability profiling experiments, each count was normalized using linear regression for spiked-in counts and quantities, then filtered for a minimal count after normalization.

For polysome profiling experiment, proportion high values were calculated as follows:

$${Proportion}_{h i g h} = \frac{c o u n {t}_{h i g h}}{c o u n {t}_{h i g h} + c o u n {t}_{l o w}}$$

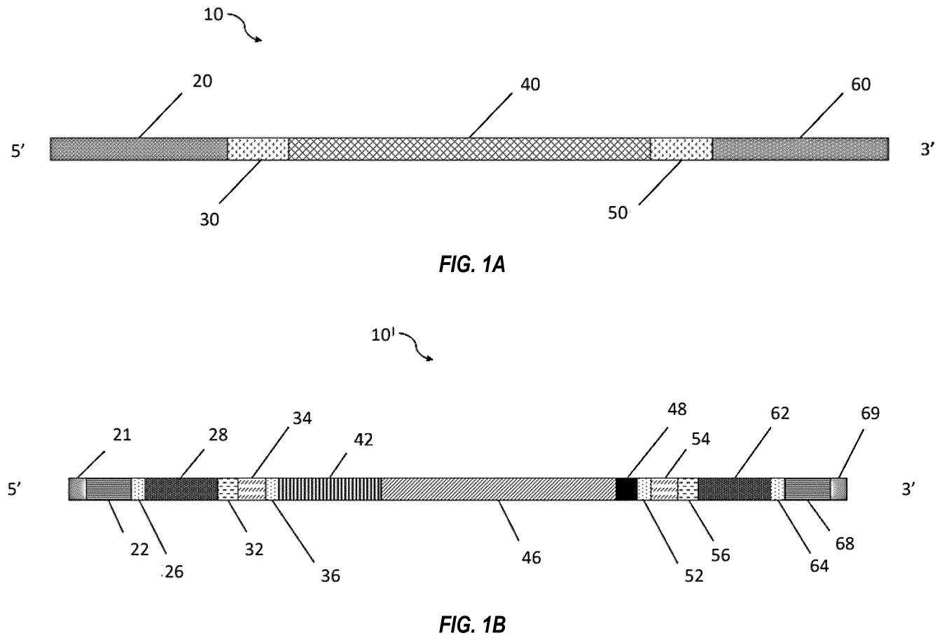

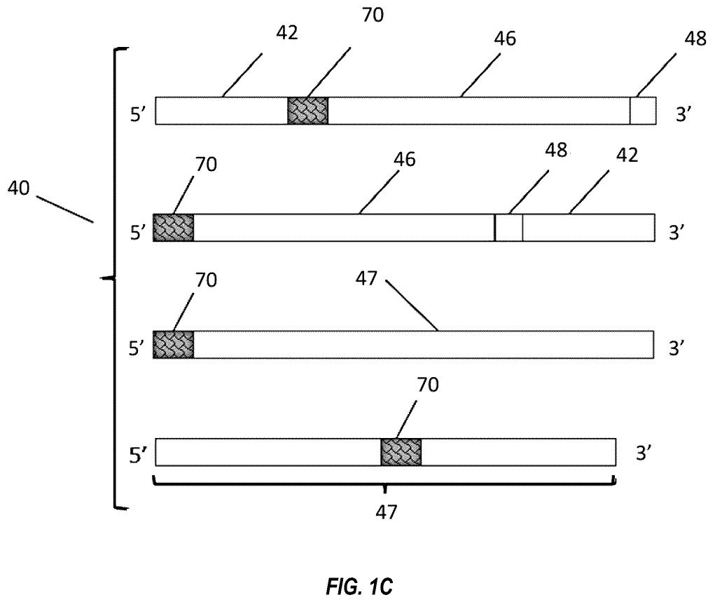

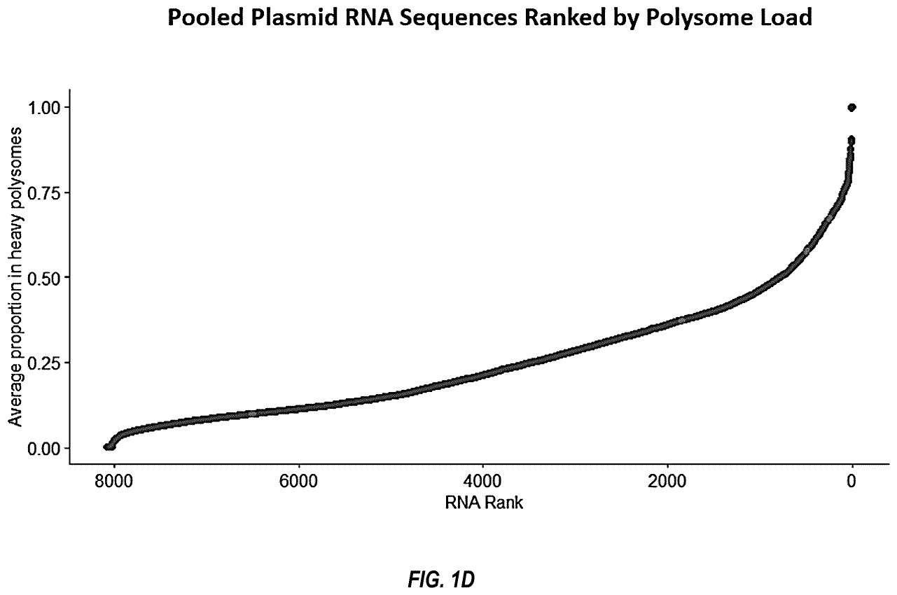

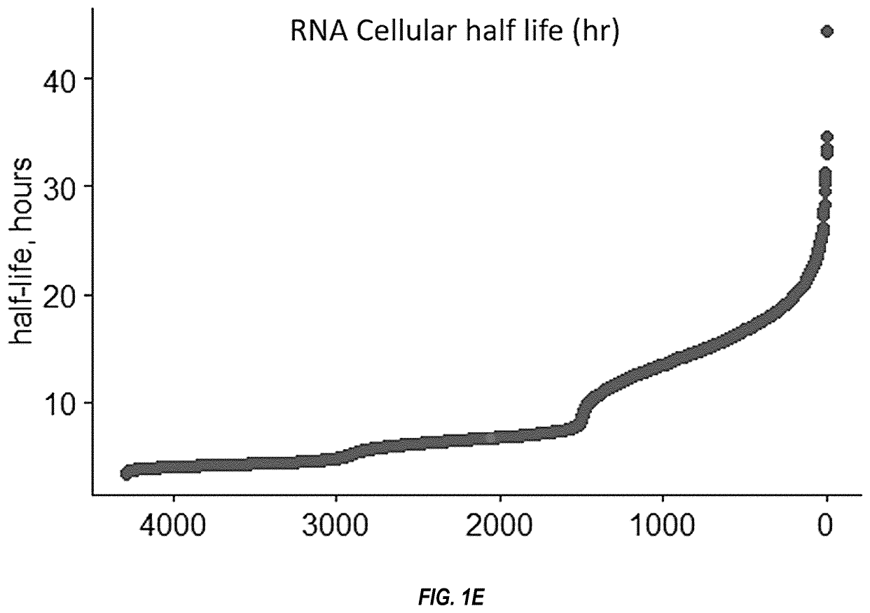

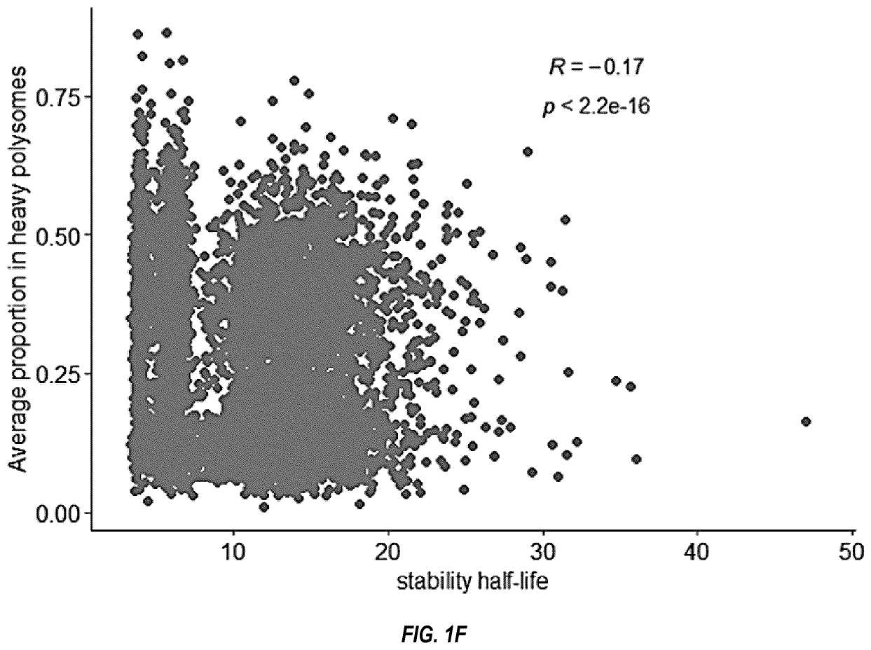

In the stability experiment, after normalization constructs were filtered for having a minimal count in at least 3 timepoints. These values were then normalized to the first time point being 1, and natural log transformed. Each remaining construct then had linear regression performed, and high confidence linear regressions were selected by filtering for a p-value of 0.05 or lower on the regression, low SEM in repeated measures, and high sequencing depth and an R² value of >0.6. Further selection criteria included having a 0.8 times polysome load as compared to a circular RNA encoding an exemplary IRES. Half-life was then calculated using the following formula:

$${t}_{1 / 2} = \frac{ln ( 10 ) - b}{a} - \frac{ln ( 20 ) - b}{a}$$

Where a is the coefficient of the linear regression and b is the intercept of the linear regression. High confidence on half-life were selected by filtering for p-value of 0.01 or lower. FIGS. 1A-1C provides the average portion in heavy polysomes and estimated half-life of circular RNAs comprising IRESes herein.

### Exemplary Engineering and Selection of Anti-CD19 Chimeric Antigen Receptor (CAR)

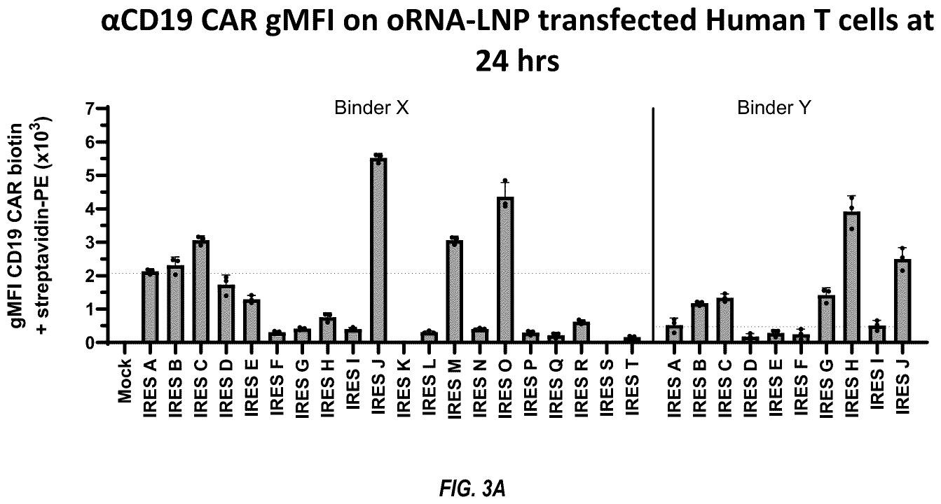

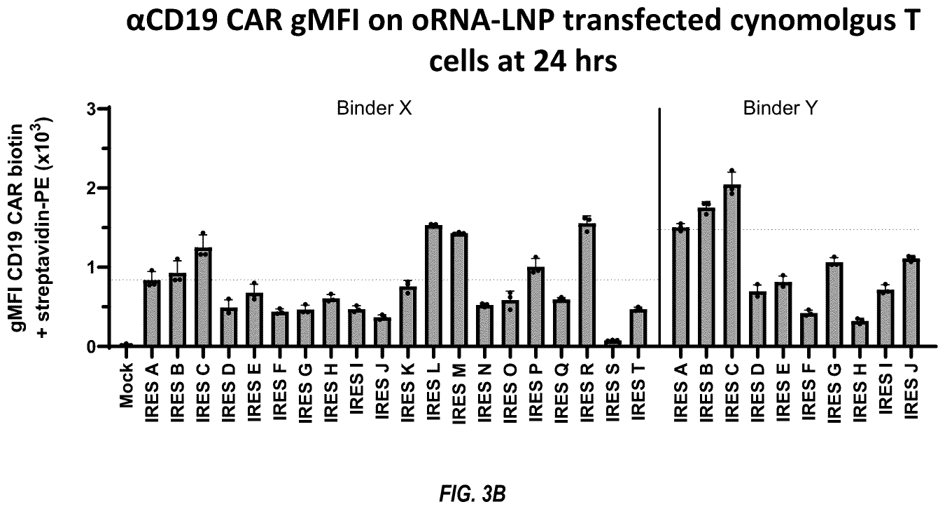

30 engineered circular RNAs encoding anti-CD19 chimeric antigen receptor (CAR) candidates were tested to express in vitro in human T cells and cynomolgus T cells. Human T cells were activated for 3 days with anti-CD3 and anti-CD28 solution and allowed to rest for 24 hours. Cynomolgus T cells were activated for 4 days with anti-CD3, anti-CD28, and/or anti-CD2 solutions and allowed to rest for 24 hours. Concurrently, the engineered circular RNA constructs were designed to encode a CD19-CD28ζ chimeric antigen receptor (CAR). For comparison purposes, “mock” T cells were not electroporated with circular RNA. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a X1ab restriction site. The circular RNAs were engineered to comprise an internal ribosome entry site (IRES). Anti-CD19 CAR sequences used comprised Binder X or Binder Y as shown in Table αα. DNA templates comprised of DNA Templates 1-30 from the table below. Human T cells were electroporated with the oRNA at 25 ng or 5 ng per 0.1×10⁶ T cells to form CAR-T cells (depicted in FIGS. 2A-2F and FIG. 3A) Cynomolgus donor T cells were transfected with the engineered circular RNAs at 200 ng per 0.1×10⁶ T cell to form CAR-T cells (depicted in FIG. 3BX). As a control, “Mock” T cells not electroporated with circular RNAs were analyzed. T cells were then allowed to rest for 24 hours after transfection. 24 and 72 hours after transfection, the CAR-T cells were counted and assessed for anti-CD19 CAR expression. Resulting oCAR-T cells were given a commercially available biotinylated soluble human CD19 detection reagent (e.g., from Miltenyi Biotec, Bergisch Gladbach) and Streptavidin conjugated to R-phycoerythrin (PE) fluorophores (e.g., from Biolegend, San Diego), or commercially available anti-cynomolgus CD19 detection reagent (e.g., from KactusBio, Waltham Massachusetts). Anti-CD19 expression was assessed using fluorescence activated cell sorting (FACS) and gating flow cytometry methods known in the art at one or more timepoints from 24 hours post transfection. CAR expression and intensity as depicted from the CD19 detection reagent used for Human and Cynomologous are shown in FIGS. 2A-2F and FIG. 3A-3B for the T cells tested.

TABLE aa

| DNA | Construct Number | | Costim | |
|---|---|---|---|---|
| Template # | from Table 5 | Binder (s) | domain | IRES |
| 1 | Construct Q | Binder X (Table 10, #17) | CD28ζ | IRES A (Table 4, #19) |
| 2 | Construct R | Binder X | CD28ζ | IRES B (Table 4, #20) |
| 3 | Construct S | Binder X | CD28ζ | IRES C (Table 4, #21) |
| 4 | Construct T | Binder X | CD28ζ. | IRES D (Table 4, #22) |
| 5 | Construct U | Binder X | CD28ζ | IRES E (Table 4, #23) |
| 6 | Construct V | Binder X | CD28ζ | IRES F (Table 4, #24) |
| 7 | Construct W | Binder X | CD28ζ | IRES G (Table 4, #25) |
| 8 | Construct X | Binder X | CD28ζ | IRES H (Table 4, #26) |
| 9 | Construct Y | Binder X | CD28ζ | IRES I (Table 4, #27) |
| 10 | Construct Z | Binder X | CD28ζ | IRES J (Table 4, #28) |
| 11 | Construct AA | Binder X | CD28ζ | IRES K (Table 4, #29) |
| 12 | Construct AB | Binder X | CD28ζ | IRES L (Table 4, #30) |
| 13 | Construct AC | Binder X | CD28ζ | IRES M (Table 4, #31) |
| 14 | Construct AD | Binder X | CD28ζ | IRES N (Table 4, #32) |
| 15 | Construct AE | Binder X | CD28ζ | IRES O (Table 4, #33) |
| 16 | Construct AF | Binder X | CD28ζ | IRES P (Table 4, #34) |
| 17 | Construct AG | Binder X | CD28ζ | IRES Q (Table 4, #35) |
| 18 | Construct AH | Binder X | CD28ζ | IRES R (Table 4, #36) |
| 19 | Construct AI | Binder X | CD28ζ | IRES S (Table 4, #37) |
| 20 | Construct AJ | Binder X | CD28ζ | IRES T (Table 4, #38) |
| 21 | Construct AK | Binder Y (Table 10, #18) | CD28ζ | IRES A |
| 22 | Construct AL | Binder Y | CD28ζ | IRES B |
| 23 | Construct AM | Binder Y | CD28ζ | IRES C |
| 24 | Construct AN | Binder Y | CD28ζ | IRES D |
| 25 | Construct AO | Binder Y | CD28ζ | IRES E |
| 26 | Construct AP | Binder Y | CD28ζ | IRES F |
| 27 | Construct AQ | Binder Y | CD28ζ | IRES G |
| 28 | Construct AR | Binder Y | CD28ζ | IRES H |
| 29 | Construct AS | Binder Y | CD28ζ | IRES I |
| 30 | Construct AT | Binder Y | CD28ζ | IRES J |

### Example 2: Production of Lipid Nanoparticle Compositions

In order to investigate lipid nanoparticle compositions for use in the delivery of circular RNA to cells, a range of formulations were prepared and tested. Specifically, the particular elements and ratios thereof in the lipid component of nanoparticle compositions were optimized.

Nanoparticles can be made in a one fluid stream or with mixing processes such as microfluidics and T-junction mixing of two fluid streams, one of which contains the circular RNA and the other has the lipid components.

Lipid compositions can be prepared, including by combining an ionizable lipid, optionally a helper lipid (such as DOPE, DSPC, or oleic acid obtainable from Avanti Polar Lipids, Alabaster, AL), a PEG lipid (such as 1,2-dimyristoyl-sn-glycerol methoxypolyethylene glycol, also known as PEG-DMG, obtainable from Avanti Polar Lipids, Alabaster, AL), and a structural lipid such as cholesterol at concentrations of about, e.g., 40 or 50 mM in a solvent, e.g., ethanol. Solutions should be refrigerated for storage at, for example, −20° C. Lipids are combined to yield desired molar ratios (see, for example, Tables 4a and 4b below) and diluted with water and ethanol to a final lipid concentration of e.g., between about 5.5 mM and about 25 mM.

### Example 2A: Exemplary LNP Formulations 1

TABLE 4a
Lipid Nanoparticle Formulations

| Formulation | |
|---|---|
| number | Description |
| 1 | Aliquots of 50 mg/mL ethanolic solutions of C12-200, DOPE, Chol and |
| | DMG-PEG2K (40:30:25:5) are mixed and diluted with ethanol to 3 mL final |
| | volume. Separately, an aqueous buffered solution (10 mM citrate/150 mM |
| | NaCl, pH 4.5) of circRNA is prepared from a 1 mg/mL stock. The lipid |
| | solution is injected rapidly into the aqueous circRNA solution and shaken to |
| | yield a final suspension in 20% ethanol. The resulting nanoparticle |
| | suspension is filtered, diafiltrated with 1 × PBS (pH 7.4), concentrated and |
| | stored at 2-8 °C |
| 2 | Aliquots of 50 mg/mL ethanolic solutions of DODAP, DOPE, cholesterol |
| | and DMG-PEG2K (18:56:20:6) are mixed and diluted with ethanol to 3 mL |
| | final volume. Separately, an aqueous buffered solution (10 mM citrate/150 |
| | mM NaCl, pH 4.5) of EPO circRNA is prepared from a 1 mg/mL stock. The |
| | lipid solution is injected rapidly into the aqueous circRNA solution and |
| | shaken to yield a final suspension in 20% ethanol. The resulting nanoparticle |
| | suspension is filtered, diafiltrated with 1 × PBS (pH 7.4), concentrated and |
| | stored at 2-8 °C. Final concentration = 1.35 mg/mL EPO circRNA |
| | (encapsulated). Zave = 75.9 nm (Dv(50) = 57.3 nm; Dv(90) = 92.1 nm). |
| 3 | Aliquots of 50 mg/mL ethanolic solutions of HGT4003, DOPE, cholesterol |
| | and DMG-PEG2K (50:25:20:5) are mixed and diluted with ethanol to 3 mL |
| | final volume. Separately, an aqueous buffered solution (10 mM citrate/150 |
| | mM NaCl, pH 4.5) of circRNA is prepared from a 1 mg/mL stock. The lipid |
| | solution is injected rapidly into the aqueous circRNA solution and shaken to |
| | yield a final suspension in 20% ethanol. The resulting nanoparticle |
| | suspension is filtered, diafiltrated with 1 × PBS (pH 7.4), concentrated and |
| | stored at 2-8 °C. |
| 4 | Aliquots of 50 mg/mL ethanolic solutions of ICE, DOPE and DMG-PEG2K |
| | (70:25:5) are mixed and diluted with ethanol to 3 mL final volume. |
| | Separately, an aqueous buffered solution (10 mM citrate/150 mM NaCl, pH |
| | 4.5) of circRNA is prepared from a 1 mg/mL stock. The lipid solution is |
| | injected rapidly into the aqueous circRNA solution and shaken to yield a |
| | final suspension in 20% ethanol. The resulting nanoparticle suspension is |
| | filtered, diafiltrated with 1 × PBS (pH 7.4), concentrated and stored at 2-8 °C. |
| 5 | Aliquots of 50 mg/mL ethanolic solutions of HGT5000, DOPE, cholesterol |
| | and DMG-PEG2K (40:20:35:5) are mixed and diluted with ethanol to 3 mL |
| | final volume. Separately, an aqueous buffered solution (10 mM citrate/150 |
| | mM NaCl, pH 4.5) of EPO circRNA is prepared from a 1 mg/mL stock. The |
| | lipid solution is injected rapidly into the aqueous circRNA solution and |
| | shaken to yield a final suspension in 20% ethanol. The resulting nanoparticle |
| | suspension is filtered, diafiltrated with 1 × PBS (pH 7.4), concentrated and |
| | stored at 2-8 °C. Final concentration = 1.82 mg/mL EPO mRNA |
| | (encapsulated). Zave = 105.6 nm (Dv(50) = 53.7 nm; Dv(90) = 157 nm). |
| 6 | Aliquots of 50 mg/mL ethanolic solutions of HGT5001, DOPE, cholesterol |
| | and DMG-PEG2K (40:20:35:5) are mixed and diluted with ethanol to 3 mL |
| | final volume. Separately, an aqueous buffered solution (10 mM citrate/150 |
| | mM NaCl, pH 4.5) of EPO circRNA is prepared from a 1 mg/mL stock. The |
| | lipid solution is injected rapidly into the aqueous circRNA solution and |
| | shaken to yield a final suspension in 20% ethanol. The resulting nanoparticle |
| | suspension is filtered, diafiltrated with 1 × PBS (pH 7.4), concentrated and |
| | stored at 2-8 °C. |
| 7 | Aliquots of 50 mg/mL ethanolic solutions of HGT5001, DOPE, cholesterol |
| | and DMG-PEG2K (35:16:46.5:2.5) are mixed and diluted with ethanol to 3 |
| | mL final volume. Separately, an aqueous buffered solution (10 mM |
| | citrate/150 mM NaCl, pH 4.5) of EPO circRNA is prepared from a 1 mg/mL |
| | stock. The lipid solution is injected rapidly into the aqueous circRNA |
| | solution and shaken to yield a final suspension in 20% ethanol. The resulting |
| | nanoparticle suspension is filtered, diafiltrated with 1 × PBS (pH 7.4), |
| | concentrated and stored at 2-8 °C. |
| 8 | Aliquots of 50 mg/mL ethanolic solutions of HGT5001, DOPE, cholesterol |
| | and DMG-PEG2K (40:10:40:10) are mixed and diluted with ethanol to 3 mL |
| | final volume. Separately, an aqueous buffered solution (10 mM citrate/150 |
| | mM NaCl, pH 4.5) of EPO circRNA is prepared from a 1 mg/mL stock. The |
| | lipid solution is injected rapidly into the aqueous circRNA solution and |
| | shaken to yield a final suspension in 20% ethanol. The resulting nanoparticle |
| | suspension is filtered, diafiltrated with 1 × PBS (pH 7.4), concentrated and |
| | stored at 2-8 °C. |
| 9 | Aliquots of 50 mg/mL ethanolic solutions of LP1, DSPC, cholesterol and |
| | DMG-PEG2K (45:9:44:2) are mixed and diluted with ethanol to 3 mL final |
| | volume. Separately, an aqueous buffered solution (50 mM Na Acetate, pH |
| | 4.5) of FLuc, hEPO, micro-dystrophin, mini-dystrophin, or dystrophin |
| | circRNA is prepared from a 1 mg/mL stock. The lipid solution is injected |
| | rapidly into the aqueous circRNA solution and shaken to yield a final |
| | suspension in 25% ethanol. The resulting nanoparticle suspension is filtered, |
| | diafiltrated with 1 × PBS (pH 7.4), concentrated and stored at 2-8 °C. |
| 10 | Aliquots of 50 mg/mL ethanolic solutions of LP1, DSPC, cholesterol and |
| | DMG-PEG2K, and DMSO solution of Ethyl Lauroyl Arginate |
| | (38.25:7.65:37.4:1.7:15) are mixed and diluted with ethanol to 3 mL final |
| | volume. Separately, an aqueous buffered solution (50 mM Bis-Tris, pH 7.0) |
| | of FLuc, hEPO, micro-dystrophin, mini-dystrophin, or dystrophin circRNA |
| | is prepared from a 1 mg/mL stock. The lipid solution is injected rapidly into |
| | the aqueous circRNA solution and shaken to yield a final suspension in 25% |
| | ethanol. The resulting nanoparticle suspension is filtered, diafiltrated with |
| | 1 × (pH 7.4), concentrated and stored at 2-8 °C. |
| 11 | Aliquots of 50 mg/mL ethanolic solutions of SM-102, DSPC, cholesterol and |
| | DMG-PEG2K (50:10:38.5:1.5) are mixed and diluted with ethanol to 3 mL |
| | final volume. Separately, an aqueous buffered solution (6.25 mM Na |
| | Acetate, pH 4.5) of FLuc, hEPO, micro-dystrophin, mini-dystrophin, or |
| | dystrophin circRNA is prepared from a 1 mg/mL stock. The lipid solution is |
| | injected rapidly into the aqueous circRNA solution and shaken to yield a |
| | final suspension in 25% ethanol. The resulting nanoparticle suspension is |
| | filtered, diafiltrated with 1 × PBS (pH 7.4), concentrated and stored at 2-8 °C. |
| 12 | Aliquots of 50 mg/mL ethanolic solutions of SM-102, DSPC, cholesterol and |
| | DMG-PEG2K, and DMSO solution of Ethyl Lauroyl Arginate |
| | (42.5:8.5:32.73:1.275:15) are mixed and diluted with ethanol to 3 mL final |
| | volume. Separately, an aqueous buffered solution (20 mM Na Acetate, pH |
| | 4.5) of FLuc, hEPO, micro-dystrophin, mini-dystrophin, or dystrophin |
| | circRNA is prepared from a 1 mg/mL stock. The lipid solution is injected |
| | rapidly into the aqueous circRNA solution and shaken to yield a final |
| | suspension in 25% ethanol. The resulting nanoparticle suspension is filtered, |
| | diafiltrated with 1 × PBS (pH 7.4), concentrated and stored at 2-8 °C. |
| 13 | Ionizable lipids, phospholipid, cholesterol, and a PEG lipid were dissolved |
| | in pure ethanol at the specified mol % ratios with a total lipid concentration |
| | of 10.8 mM. A 0.10 mg/mL mRNA solution was prepared using acidic buffer |
| | (pH 4.0-5.0) containing mRNA encoding VHH. The nucleotide and lipid |
| | solutions were mixed at a 3:1 volume ratio using the NanoAssemblr |
| | microfluidic system at a 12 mL/min total flow rate resulting in rapid mixing |
| | and self-assembly of LNPs. Formulations were further dialyzed against PBS |
| | (pH 7.4) overnight at 4 °C., concentrated using centrifugal filtration and |
| | filtered (0.2 μm pore size). The particle size and polydispersity index (PDI) |
| | of formulations was measured by dynamic light scattering (DLS) using a |
| | Zetasizer Ultra (Malvern Panalytical). RNA encapsulation efficiency (EE %) |
| | was determined by Ribogreen assay. |
| 14 | Ionizable lipids, phospholipid, cholesterol, PEG-lipid were dissolved in pure |
| | ethanol at the specified mol % ratio with a total lipid concentration of 10.8 |
| | mM. A 0.10-0.20 mg/mL reporter mRNA + barcoded DNA solution was |
| | prepared using acidic buffer (pH 4.0-5.0) at a ratio of 1:1, 10:1, or 100:1 |
| | ratios by weight. The nucleotide and lipid solutions were mixed at a 3:1 |
| | volume ratio using the NanoAssemblr microfluidic system at a 12 mL/min |
| | total flow rate resulting in rapid mixing and self-assembly of LNPs. |
| | Formulations were further dialyzed against PBS (pH 7.4) overnight at 4 °C., |
| | and buffer exchanged into a sucrose-containing Tris-HCl cryoprotectant |
| | buffer for subsequent storage at −80 °C. The individual particle sizes of |
| | formulations was measured by dynamic light scattering (DLS) using a |
| | Zetasizer Ultra (Malvern Panalytical). RNA encapsulation efficiency was |
| | determined by Ribogreen assay. |

In some embodiments, the transfer vehicle has a formulation as described in Table 4b.

TABLE 4b
Exemplary Lipid Vehicle Formulations

| Ionizable Lipid | Phospholipid | Sterol | PEG Lipid |
|---|---|---|---|
| (mol %) | (mol %) | (mol %) | (mol %) |
| 40 | 20 | 38.5 | 1.5 |
| 45 | 15 | 38.5 | 1.5 |
| 50 | 10 | 38.5 | 1.5 |
| 55 | 5 | 38.5 | 1.5 |
| 60 | 5 | 33.5 | 1.5 |
| 45 | 20 | 33.5 | 1.5 |
| 50 | 20 | 28.5 | 1.5 |
| 55 | 20 | 23.5 | 1.5 |
| 60 | 20 | 18.5 | 1.5 |
| 40 | 15 | 43.5 | 1.5 |
| 50 | 15 | 33.5 | 1.5 |
| 55 | 15 | 28.5 | 1.5 |
| 60 | 15 | 23.5 | 1.5 |
| 40 | 10 | 48.5 | 1.5 |
| 45 | 10 | 43.5 | 1.5 |
| 55 | 10 | 33.5 | 1.5 |
| 60 | 10 | 28.5 | 1.5 |
| 40 | 5 | 53.5 | 1.5 |
| 45 | 5 | 48.5 | 1.5 |
| 50 | 5 | 43.5 | 1.5 |
| 40 | 20 | 40 | 0 |
| 45 | 20 | 35 | 0 |
| 50 | 20 | 30 | 0 |
| 55 | 20 | 25 | 0 |
| 60 | 20 | 20 | 0 |
| 40 | 15 | 45 | 0 |
| 48.5 | 10 | 40 | 1.5 |
| 48.5 | 10 | 39 | 2.5 |
| 33 | 40 | 25 | 2 |

In certain embodiments, the lipid nanoparticle formulation comprises one or more ionizable lipids at a total molar ratio disclosed above, wherein the ionizable lipid is selected from those disclosed in any one of US 2023/0053437; US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; WO 2019/089828A1; WO2023044343A1; WO2023044333A1; WO2023122752A1; WO2024044728A1; WO2023196931A1; and PCT/US2024/019990 (each of which is incorporated by reference herein, in its entirety), or any other ionizable lipid of the present disclosure, or those otherwise incorporated by reference herein. In certain embodiments, the ionizable lipid is one of the following:

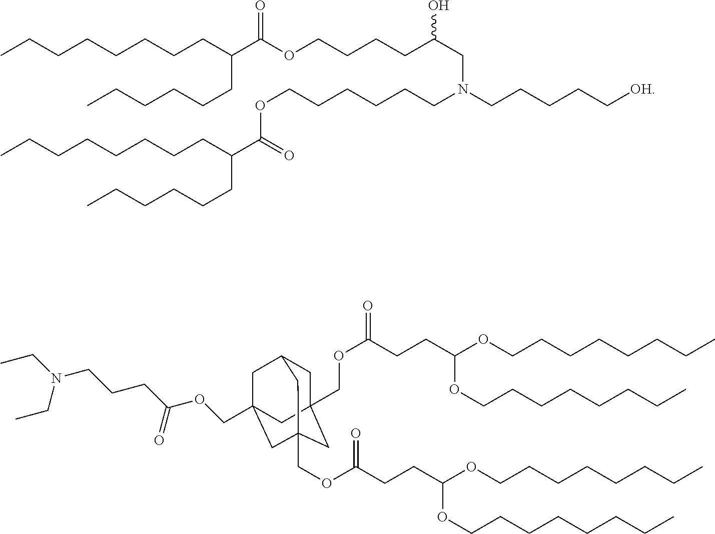 [SMILES: CCCCCCCCC(CCCCCC)C(=O)OCCCCCCN(CCCCCO)CC(O)CCCCOC(=O)C(CCCCCC)CCCCCCCC ; CCCCCCCCOC(CCC(=O)OCC12CC3CC(COC(=O)CCCN(CC)CC)(C1)CC(COC(=O)CCC(OCCCCCCCC)OCCCCCCCC)(C3)C2)OCCCCCCCC]

In certain embodiments, the lipid nanoparticle formulation comprises one or more phospholipids at a total molar ratio disclosed above. In certain embodiments the phospholipid is selected from DOPE, DSPC, sphingomyelin and mixtures thereof.

In certain embodiments, the lipid nanoparticle formulation comprises one or more sterols at a total molar ratio disclosed above. In certain embodiments the sterol is cholesterol.

In certain embodiments, the lipid nanoparticle formulation comprises one or more PEG lipids at a total molar ratio disclosed above. In certain embodiments the PEG lipid is selected from DMG-PEG2k and DSPE-PEG2k. In certain embodiments, the PEG lipid is disclosed in WO2024044728A1, which is incorporated by reference herein, in its entirety.

For nanoparticle compositions including circRNA, solutions of the circRNA at concentrations of 0.1 mg/ml in deionized water are diluted in a buffer, e.g., 50 mM sodium citrate buffer at a pH between 3 and 4 to form a stock solution. Alternatively, solutions of the circRNA at concentrations of 0.15 mg/ml in deionized water are diluted in a buffer, e.g., 6.25 mM sodium acetate buffer at a pH between 3 and 4.5 to form a stock solution.

Nanoparticle compositions including a circular RNA and a lipid component are prepared by combining the lipid solution with a solution including the circular RNA at lipid component to circRNA wt:wt ratios between about 5:1 and about 50:1. The lipid solution is rapidly injected using, e.g., a NanoAssemblr microfluidic based system at flow rates between about 10 ml/min and about 18 ml/min or between about 5 ml/min and about 18 ml/min into the circRNA solution, to produce a suspension with a water to ethanol ratio between about 1:1 and about 4:1.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa or 20 kDa. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/ml to 0.15 mg/ml are generally obtained.

The method described above induces nano-precipitation and particle formation.

Alternative processes including, but not limited to, T-junction and direct injection, may be used to achieve the same nano-precipitation.

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of circRNA in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of circRNA in the nanoparticle composition can be calculated based on the extinction coefficient of the circRNA used in the composition and on the difference between the absorbance at a wavelength of, for example, 260 nm and the baseline value at a wavelength of, for example, 330 nm.

A QUANT-IT™ RIBOGREEN® RNA assay (Invitrogen Corporation Carlsbad, CA) can be used to evaluate the encapsulation of circRNA by the nanoparticle composition. The samples are diluted to a concentration of approximately 5 g/mL or 1 g/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free circRNA is determined by dividing the fluorescence intensity of the intact sample (without addition of Triton X-100) by the fluorescence value of the disrupted sample (caused by the addition of Triton X-100).

As used herein, Formulation A and Formulation B have the following identities:

### Formulation A:

| Ionizable Lipid | Phospholipid | Sterol | PEG Lipid |
|---|---|---|---|
| (mol %) | (mol %) | (mol %) | (mol %) |
| O-1 | DSPC | Cholesterol | DMG-PEG2k |
| 50 | 10 | 38.5 | 1.5 |

### Formulation B:

| Ionizable Lipid | Phospholipid | Sterol | PEG Lipid |
|---|---|---|---|
| (mol %) | (mol %) | (mol %) | (mol %) |
| AX-6 | Egg | Cholesterol | DMG-PEG2k |
| | Sphingomyelin (ESM) | | |
| 33 | 40 | 25 | 2 |

### Example 2B: Exemplary LNP Formulations 2

Ionizable lipids, phospholipid, cholesterol, and PEG lipid were dissolved in pure ethanol at the specified mol % ratios with a total lipid concentration of 15.9 mM. A 0.075 mg/mL mRNA solution was prepared using acidic buffer (pH 4.0-5.0) containing circular RNA encoding VHH or a CAR protein. The nucleotide and lipid solutions were mixed at a 3:1 volume ratio using the Knauer mixing system at a 30-40 mL/min total flow rate resulting in rapid mixing and self-assembly of LNPs. Formulations were further buffer exchanged against cryopreservation buffer using a Repligen TFF system and concentrated prior to sterile filtration (0.2 μm pore size). The particle size and polydispersity index (PDI) of formulations was measured by dynamic light scattering (DLS) using a Zetasizer Ultra (Malvern Panalytical). RNA encapsulation efficiency (EE %) was determined by Ribogreen assay.

TABLE XA
In Vivo VHH and ORNA CAR Formulations

| | | | | | | N:P/ | Size | | |
|---|---|---|---|---|---|---|---|---|---|
| Form. | Payload | IL | PL | PEG | Sterol | Buffer | (nm) | PDI | ee % |
| F-2 | VHH | AX-6 | ESM | PEG2k-DMG | Chol. | 8 | 86.6 | 0.07 | 90.1 |
| | oRNA | 33 | 40 | 2 | 25 | Y | | | |
| F-4 | CAR | AX-6 | ESM | PEG2k-DMG | Chol. | 8 | 80.9 | 0.07 | 92.3 |
| | oRNA | 33 | 40 | 2 | 25 | Y | | | |
| F-S1 | VHH | AX-6 | ESM | PEG2k-DMG | Chol. | 8 | 77.1 | 0.05 | 96.1 |
| | oRNA | 33 | 40 | 2 | 25 | Y | | | |
| F-S2 | CAR | AX-6 | ESM | PEG2k-DMG | Chol. | 8 | 90.1 | 0.10 | 90.4 |
| | oRNA | 33 | 40 | 2 | 25 | Y | | | |

Buffer X: 25 mM Sodium Acetate, pH 5.0; Buffer Y: 50 mM Citrate, pH 4.0; ESM = egg sphingomyelin; PEG2k-DMG = 1,2-dimyristoyl-rac-glycero-3-methoxypolyethylene glycol-2000; Chol = cholesterol

### Example 2C: Exemplary LNP Formulations 3-CD19

Ionizable lipids, phospholipid, cholesterol, and PEG lipid were dissolved in pure ethanol at the specified mol % ratios with a total lipid concentration of 10.8 mM. A 0.05 mg/mL RNA solution was prepared using acidic buffer (pH 4.0-5.0) containing circular RNA encoding a VHH or a CAR protein or linear mRNA encoding a CAR protein. The nucleotide and lipid solutions were mixed at a 3:1 volume ratio using either the Knauer mixing system at a 20-40 mL/min total flow rate or the NanoAssemblr Ignite+ at a flow rate of 12 mL/min, resulting in rapid mixing and self-assembly of LNPs. Formulations were further buffer exchanged against cryopreservation buffer (20 mM Tris HCl, 8% Sucrose, pH 8) using a Repligen TFF system and concentrated prior to sterile filtration (0.2 μm pore size). The particle size and polydispersity index (PDI) of formulations was measured by dynamic light scattering (DLS) using a Zetasizer Ultra (Malvern Panalytical). RNA encapsulation efficiency (EE %) was determined by Ribogreen assay.

TABLE XB
oRNA and mRNA CAR

| | | | | | N:P/ | Size | | |
|---|---|---|---|---|---|---|---|---|
| Form. | IL | PL | PEG | Sterol | Buffer | (nm) | PDI | ee % |
| F-C1 | AX-6 | ESM | PEG2k-DMG | Chol. | 8 | — | — | — |
| | 33 | 40 | 2 | 25 | Y | | | |
| F-C2 | AX-6 | ESM | PEG2k-DMG | Chol. | 8 | 74.8 | 0.003 | 96.1 |
| | 33 | 40 | 2 | 25 | Y | | | |
| F-C3 | AX-6 | ESM | PEG2k-DMG | Chol. | 8 | 73.9 | 0.011 | 95.7 |
| | 33 | 40 | 2 | 25 | Y | | | |
| F-C4 | AX-6 | ESM | PEG2k-DMG | Chol. | 8 | 75.1 | 0.015 | 94.7 |
| | 33 | 40 | 2 | 25 | Y | | | |
| F-C5 | AX-6 | ESM | PEG2k-DMG | Chol. | 8 | 70.5 | 0.006 | 96.0 |
| | 33 | 40 | 2 | 25 | Y | | | |
| F-C6 | AX-6 | ESM | PEG2k-DMG | Chol. | 8 | 70.3 | 0.026 | 95.0 |
| | 33 | 40 | 2 | 25 | Y | | | |
| F-C7 | AX-6 | ESM | PEG2k-DMG | Chol. | 8 | 66.7 | 0.071 | 96.1 |
| | 33 | 40 | 2 | 25 | Y | | | |
| F-C8 | AX-6 | ESM | PEG2k-DMG | Chol. | 8 | 74.8 | 0.024 | 95.6 |
| | 33 | 40 | 2 | 25 | Y | | | |
| F-C9 | AX-6 | ESM | PEG2k-DMG | Chol. | 8 | 75.2 | 0.023 | 96.4 |
| | 33 | 40 | 2 | 25 | Y | | | |
| F-C10 | AX-6 | ESM | PEG2k-DMG | Chol. | 8 | 74.1 | 0.009 | 96.3 |
| | 33 | 40 | 2 | 25 | Y | | | |
| F-C11 | AX-6 | ESM | PEG2k-DMG | Chol. | 8 | 74.7 | 0.022 | 97.0 |
| | 33 | 40 | 2 | 25 | Y | | | |
| F-C12 | AX-6 | ESM | PEG2k-DMG | Chol. | 8 | 74.3 | 0.023 | 95.4 |
| | 33 | 40 | 2 | 25 | Y | | | |
| F-C13 | AX-6 | ESM | PEG2k-DMG | Chol. | 8 | 72.6 | 0.084 | 94.4 |
| | 33 | 40 | 2 | 25 | Y | | | |
| F-C14 | AX-6 | ESM | PEG2k-DMG | Chol. | 8 | 72.5 | 0.112 | 94.2 |
| | 33 | 40 | 2 | 25 | Y | | | |
| F-C15 | AX-6 | ESM | PEG2k-DMG | Chol. | 8 | 72.1 | 0.099 | 94.8 |
| | 33 | 40 | 2 | 25 | Y | | | |
| F-C16 | AX-6 | ESM | PEG2k-DMG | Chol. | 8 | 74.3 | 0.093 | 93.4 |
| | 33 | 40 | 2 | 25 | Y | | | |
| F-C17 | AX-6 | ESM | PEG2k-DMG | Chol. | 8 | 72.4 | 0.109 | 94.5 |
| | 33 | 40 | 2 | 25 | Y | | | |

Buffer X: 25 mM Sodium Acetate, pH 5.0; Buffer Y: 50 mM Citrate, pH 4.0; ESM = egg sphingomyelin; PEG2k-DMG = 1,2-dimyristoyl-rac-glycero-3-methoxypolyethylene glycol-2000; Chol = cholesterol

### Example 3: Synthesis of Exemplary Ionizable Lipids

### Example 2.1 Synthesis of heptadecan-9-yl 8-((3-hydroxypropyl)(2-hydroxytetradecyl)amino)octanoate (Table 8, Lipid 1)

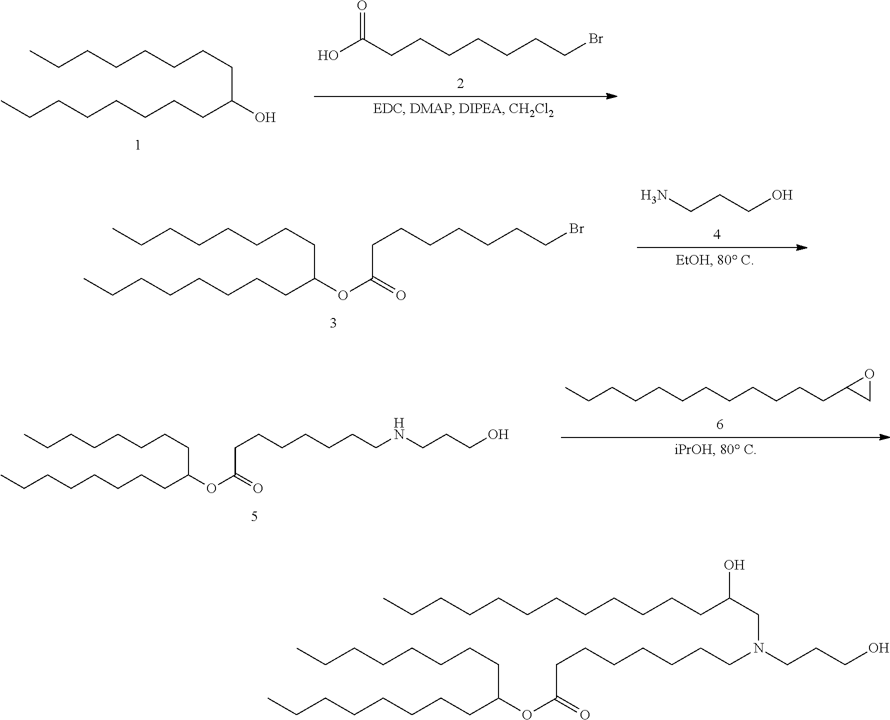

### Example 2.1.1 Synthesis of heptadecan-9-yl 8-bromooctanoate (3)

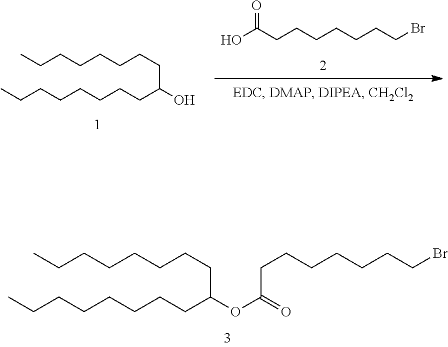

To a mixture of 8-bromooctanoic acid 2 (10 g, 44.82 mmol) and heptadecan-9-ol 1 (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated, and the crude residue was purified by flash chromatography (SiO₂: Hexane=100% to 30% of EtOAc in Hexane) and colorless oil product 3 was obtained (5 g, 29%).

¹H NMR (300 MHz, CDCl3): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H), 0.87 (t, J=6.7 Hz, 6H).

### Example 2.1.2 Synthesis of heptadecan-9-yl 8-((3-hydroxypropyl)amino)octanoate (5)

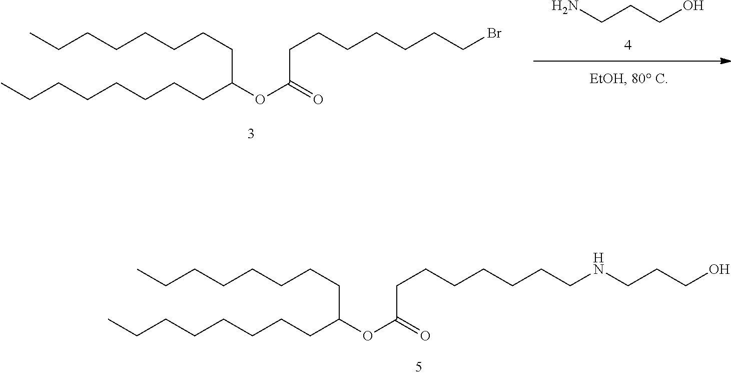 [SMILES: NCCCO ; CCCCCCCCC(CCCCCCCC)OC(=O)CCCCCCCBr ; CCCCCCCCC(CCCCCCCC)OC(=O)CCCCCCCNCCCO]

A solution of 1-octylnonyl 8-bromooctanoate 3 (7.4 g, 16.03 mmol) in EtOH (200 mL) was added 3-amino-1-propanol 4 (24.4 mL, 320 mmol) and the reaction solution was heated at 70° C. overnight. MS showed the expected product: [APCI]: [MH]⁺456.4. After concentration of the reaction mixture, the crude residue was dissolved in methyl tert-butyl ether (500 mL), washed with sat. NaHCO₃, water and Brine. The organic layer was dried over anhydrous Na₂SO₄. The solvent was evaporated, and the crude residue was purified by flash chromatography (SiO₂: CH₂Cl₂=100% to 10% of MeOH in CH₂Cl₂ with 1% NH₄OH) and colorless oil product 5 was obtained (6.6 g, 88%).

¹H NMR (300 MHz, CDCl₃): δ ppm 4.84 (m, 1H), 3.80 (t, J=5.5 Hz, 2H), 2.87 (t, J=5.76 Hz, 2H), 2.59 (t, J=7.2 Hz, 2H), 2.27 (t, J=7.6 Hz, 2H), 1.68 (m, 2H), 1.62 (m, 2H), 1.5-1.4 (m, 5H), 1.35-1.2 (m, 32H), 0.87 (t, J=6.7 Hz, 6H). MS (APCI+): 456.4 (M+1).

### Example 2.1.3 Synthesis of heptadecan-9-yl 8-((3-hydroxypropyl)(2-hydroxytetradecyl)amino)octanoate (7)

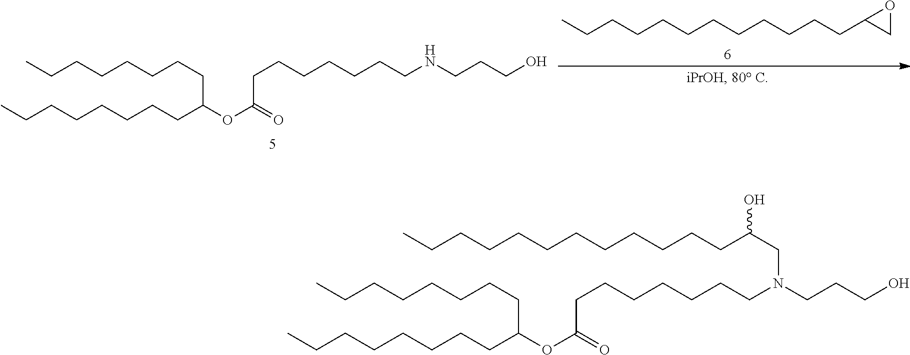 [SMILES: CCCCCCCCCCCCC1CO1 ; CCCCCCCCC(CCCCCCCC)OC(=O)CCCCCCCNCCCO ; CCCCCCCCCCCCC(O)CN(CCCO)CCCCCCCC(=O)OC(CCCCCCCC)CCCCCCCC]

A mixture of compound 5 (6.6 g, 14.5 mmol) and 1,2-epoxytetradecane (3.68 g, 17.4 mmol) in isopropanol (150 mL) was heated to reflux for overnight. MS showed the expected product: [APCI]: [MH]⁺668.6. The reaction mixture was concentrated, and crude product was purified flash chromatography (SiO₂: CH₂Cl₂=100% to 10% of MeOH in CH₂Cl₂ with 1% NH₄OH) to obtained Lipid 10e-1 as colorless oil (6.34 g, 65%).

1H NMR (300 MHz, CDCl₃): δ ppm 4.85 (m, 1H), 3.76 (t, J=5.49 Hz, 2H), 3.68 (m, 1H), 2.75 (m, 1H), 2.59 (m, 2H), 2.38 (m, 3H), 2.27 (m, 2H), 1.58-1.68 (m, 2H), 1.48 (m, 6H), 1.24 (m, 56H), 0.87 (m, 9H). MS (APCI+): 668.6 (M+1).

### Example 2.2 Synthesis of Di(undecan-3-yl) 8,8′-((3-hydroxypropyl)azanediyl)bis(7-hydroxyoctanoate) (Table 8, Lipid 7)

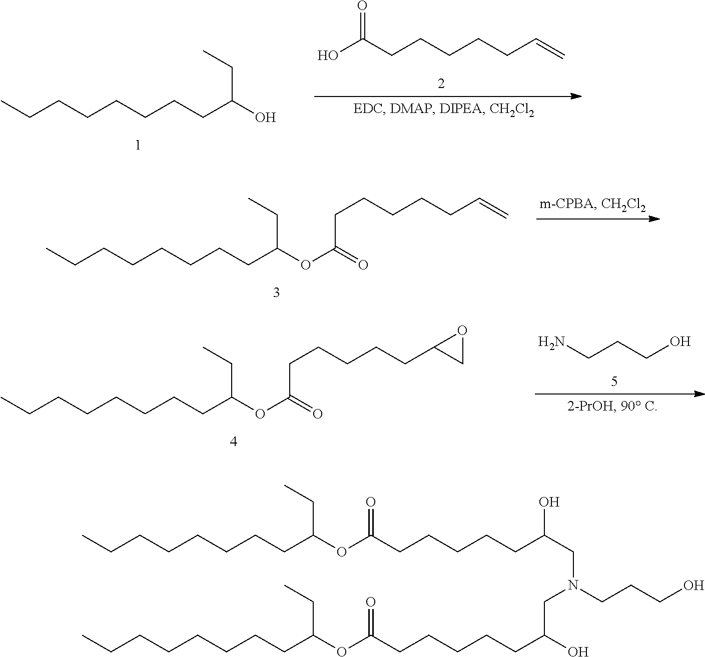

### Example 2.2.1 Synthesis of undecan-3-yl oct-7-enoate (3)

To a mixture of oct-7-enoic acid 2 (10 g, 70.3 mmol) and undecan-3-ol 1 (10 g, 58.6 mmol) in CH₂Cl₂ (300 mL) was added DMAP (1.4 g, 11.6 mmol), DIPEA (40 mL, 232 mmol) and EDC (16.9 g, 87.9 mmol). The reaction was stirred at room temperature overnight. After concentration of the reaction mixture, the crude residue was dissolved in tert-butylmethyl ether (500 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 20% of EtOAc in Hexane) and colorless oil product 3 was obtained (17.2 g, 98%).

¹H NMR (300 MHz, CDCl₃): δ ppm 5.88-5.72 (m, 1H), 5.02-4.91 (m, 1H), 4.80 (m, 1H), 2.28 (t, J=7.4 Hz, 2H), 2.05-2.03 (m, 2H), 1.62-1.49 (m, 6H), 1.37-1.25 (m, 16H), 0.87 (t, J=7.4 Hz, 6H).

### Example 2.2.2 Synthesis of undecan-3-yl 6-(oxiran-2-yl)hexanoate (4)

To a mixture of undecan-3-yl oct-7-enoate 3 (17.2 g, 58.1 mmol) in CH₂Cl₂ (300 mL) was added meta-chloroperoxybenzoic acid (mCPBA, <77%) (19.5 g, 87 mmol) in one portion at 0° C. ice-water bath. The reaction was stirred at room temperature overnight. The white precipitate (meta-benzoic acid) was filtered and the filtrate was diluted with CH2C12 (200 mL), washed with 10% Na₂S₂O₃, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 3 was obtained (17.1 g, 97%).

¹H NMR (300 MHz, CDCl3): δ ppm 4.80 (m, 1H), 2.89-2.86 (m, 1H), 3.39 (t, J=7.0 Hz, 2H), 2.74 (t, J=4.7 Hz, 1H), 2.47 (dd, J=4.9, 2.2 Hz, 1H), 2.28 (t, J=7.4 Hz, 1H), 1.74-1.46 (m, 10H), 1.35-1.2 (m, 13H) 0.87 (m, 6H).

### Example 2.2.3 Synthesis of Di(undecan-3-yl) 8,8′-((3-hydroxypropyl)azanediyl)bis(7-hydroxyoctanoate) (Table 8, lipid 7)

A solution of undecan-3-yl 6-(oxiran-2-yl)hexanoate 4 (8 g, 25.6 mmol) in isopropanol (50 mL) was added 3-amino-1-propanol (769.1 mg, 10.2 mmol) and the reaction solution was heated at 90° C. overnight. MS showed the expected product: [APCI]: [MH]⁺700.6. After concentration of the reaction mixture, the crude residue was purified by flash chromatography (SiO₂: CH₂Cl₂=100% to 10% of MeOH in CH₂Cl₂) and colorless oil product was obtained (5.1 g, 71%).

1H NMR (300 MHz, CDCl₃): δ ppm 4.81 (m, 2H), 3.80 (m, 2H), 3.73 (m, 2H), 2.78 (m, 2H), 2.52-2.43 (m, 4H), 2.28 (t, J=7.3 Hz, 2H), 1.68-1.48 (m, 15H), 1.35-1.17 (m, 37H), 0.88-0.83 (m, 12H). MS (APCI+): 700.6 (M+1).

### Example 2.3 Synthesis of ((3-(2-methyl-1H-imidazol-1-yl)propyl)azanediyl)bis(hexane-6,1-diyl) bis(2-hexyldecanoate) (Table 8, lipid 129) and ((3-(1H-imidazol-1-yl)propyl)azanediyl)bis(hexane-6,1-diyl) bis(2-hexyldecanoate) (Table 8, lipid 128)

In a 100 mL round bottom flask connected with condenser, 3-(1H-imidazol-1-yl)propan-1-amine (100 mg, 0.799 mmol) or 3-(2-methyl-1H-imidazol-1-yl)propan-1-amine (0.799 mmol), 6-bromohexyl 2-hexyldecanoate (737.2 mg, 1.757 mmol), potassium carbonate (485 mg, 3.515 mmol) and potassium iodide (13 mg, 0.08 mmol) were mixed in acetonitrile (30 mL), and the reaction mixture was heated to 80° C. for 48 h. The mixture was cooled to room temperature and was filtered through a pad of Celite. The filtrate was diluted with ethyl acetate. After washing with water, brine and dried over anhydrous sodium sulfate. The solvent was evaporated and the crude residue was purified by flash chromatography (SiO₂: CH₂Cl₂=100% to 10% of methanol in CH₂Cl₂) and colorless oil product was obtained (92 mg, 15%). Molecular formula of ((3-(1H-imidazol-1-yl)propyl)azanediyl)bis(hexane-6,1-diyl) bis(2-hexyldecanoate)) is C₅₀H₉₅N₃O₄ and molecular weight (Mw) is 801.7.

Reaction scheme for synthesis of ((3-(1H-imidazol-1-yl)propyl)azanediyl)bis(hexane-6,1-diyl) bis(2-hexyldecanoate)) (Table 8, lipid 128).

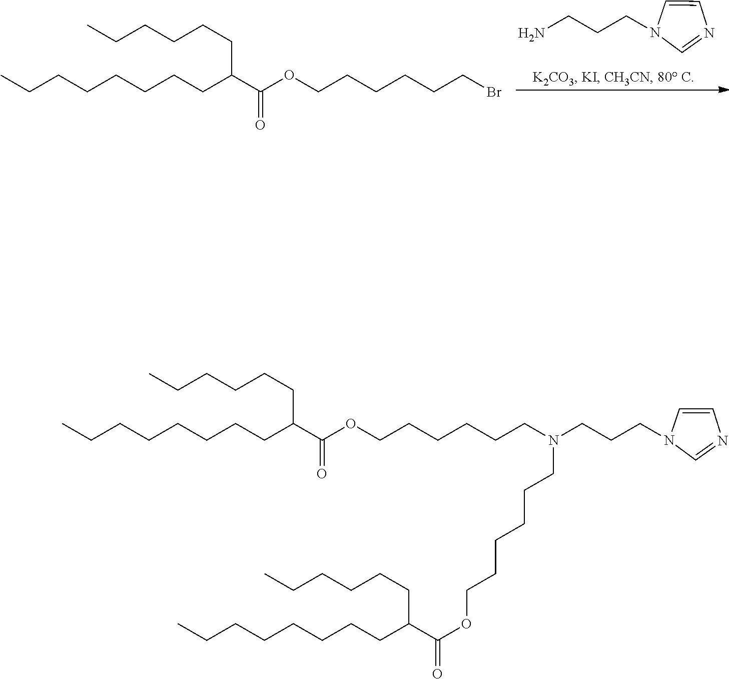 [SMILES: CCCCCCCCC(CCCCCC)C(=O)OCCCCCCBr ; NCCCn1ccnc1 ; CCCCCCCCC(CCCCCC)C(=O)OCCCCCCN(CCCCCCOC(=O)C(CCCCCC)CCCCCCCC)CCCn1ccnc1]

### Example 4: Synthesis of Additional Exemplary Ionizable Lipids

### Synthesis of Exemplary Intermediates

### Synthesis of ((1s,3s,5s)-adamantane-1,3,5-triyl)trimethanol (L150-1)

Intermediate L150-1 can be made by one of the methods generally outlined below.

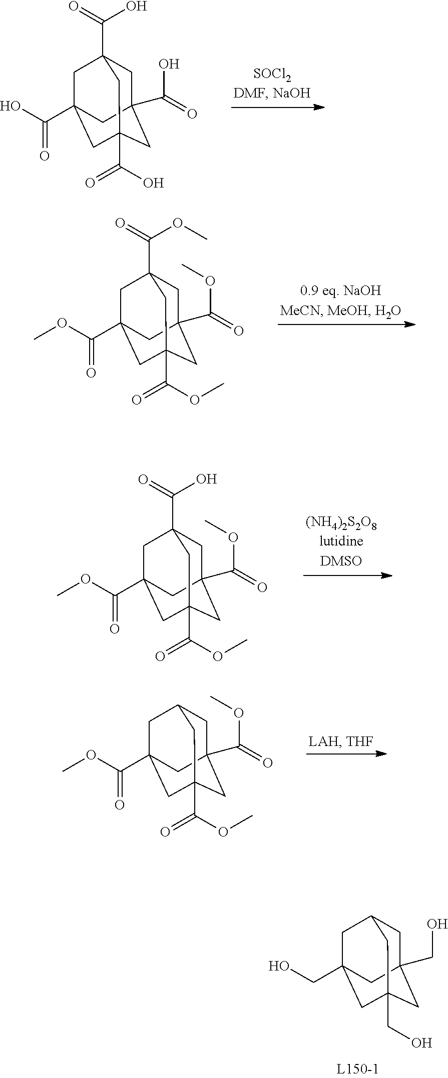

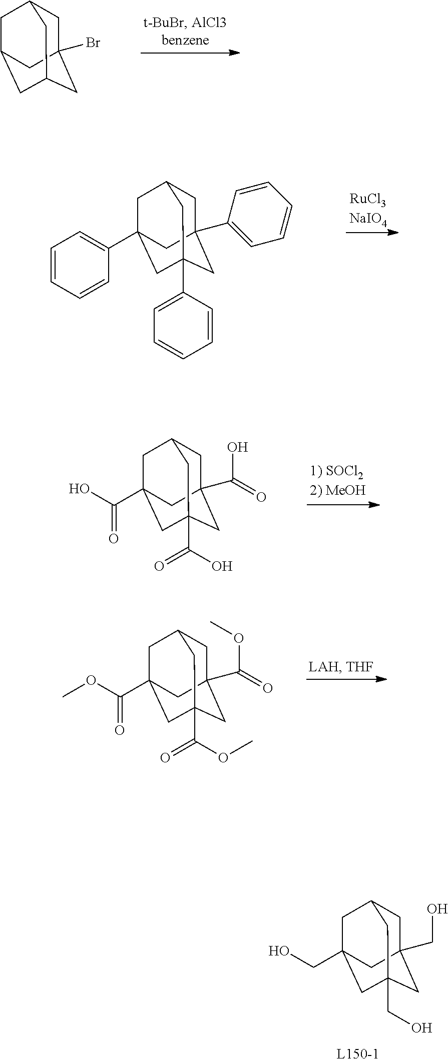

### Synthesis of 3,3′,3″-((1s,3s,5s)-adamantane-1,3,5-triyl)tris(propan-1-ol)

3,3′,3″-((1s,3s,5s)-adamantane-1,3,5-triyl)tris(propan-1-ol) can be made by a method generally outlined below.

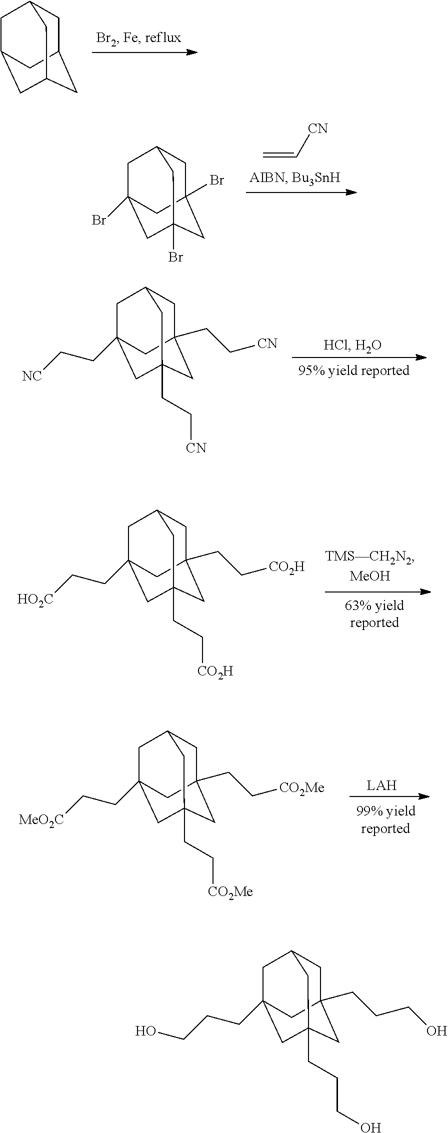

### Synthesis of 2,2′,2″-((1s,3s,5s)-adamantane-1,3,5-triyl)tris(ethan-1-ol)

2,2′,2″-((1s,3s,5s)-adamantane-1,3,5-triyl)tris(ethan-1-ol) can be made by a method generally outlined below.

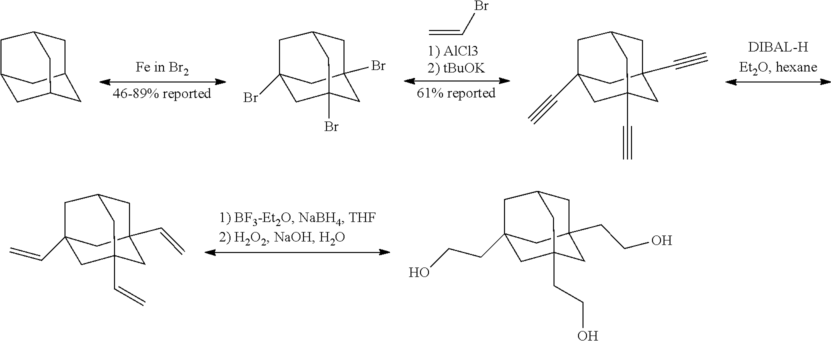

### Synthetic Scheme for Compound AX-3

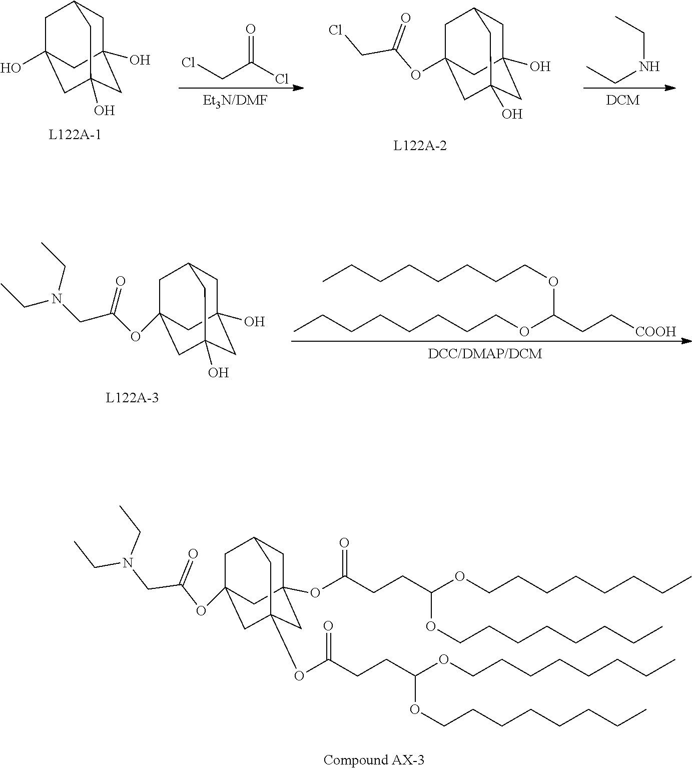

### Synthesis of (1R,3R,5S)-3,5-dihydroxyadamantan-1-yl 2-chloroacetate (L122A-2)

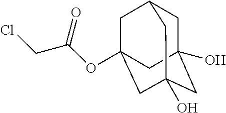 [SMILES: O=C(CCl)OC12CC3CC(O)(CC(O)(C3)C1)C2]

A solution of L122A-1 (5.0 g, 27.14 mmol) in DMF (100 mL) was heated to 45° C. under nitrogen atmosphere. Triethylamine (11.0 mL, 81.4 mmol) and chloroacetyl chloride (5.4 mL, 67.9 mmol) were added portion wise to the above solution and the reaction was monitored by TLC. Upon consumption of the starting material, the reaction mixture was concentrated under reduced pressure and the crude was purified by flash chromatography (SiO₂: 0-100% ethyl acetate in hexanes gradient and then 0-5% methanol in dichloromethane gradient) to yield L122A-2 as beige solid (3.01 g, 43%); ¹HNMR (CDCl₃) δ 3.98 (s, 2H), 2.42 (p, J=3.2 Hz, 1H), 2.09 (s, 4H), 1.98 (d, J=3.1 Hz, 2H), 1.82-1.69 (m, 2H), 1.68-1.59 (m, 4H).

### Synthesis of (1R,3R,5S)-3,5-dihydroxyadamantan-1-yl diethylglycinate (L122A-3)

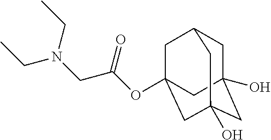 [SMILES: CCN(CC)CC(=O)OC12CC3CC(O)(CC(O)(C3)C1)C2]

A mixture of L122A-2 (3.00 g, 11.51 mmol) and diethylamine (12.0 mL, 115.1 mmol) in DCM (50 mL) was stirred at room temperature for 48 h. The mixture was concentrated under reduced pressure and the crude was purified by flash chromatography (SiO₂: 0-5% methanol in dichloromethane gradient) to afford L122A-3 as brownish solid (2.47 g, 72%); ¹HNMR (CDCl₃) δ 3.24 (s, 2H), 2.64 (q, J=6.5 Hz, 4H), 2.47-2.32 (m, 1H), 2.10 (s, 4H), 1.98 (s, 2H), 1.81-1.66 (m, 6H), 1.06 (t, J=7.1 Hz, 6H); CIMS m/z [M+H]298.2.

### Synthesis of (1R,3S,5S)-5-((diethylglycyl)oxy)adamantane-1,3-diyl bis(4,4-bis(octyloxy) butanoate) (Compound AX-3)

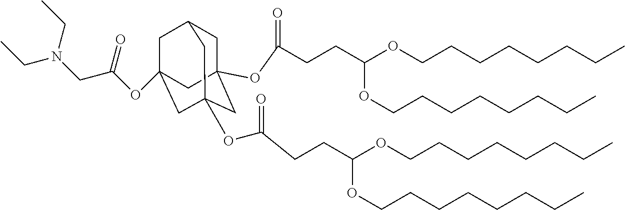 [SMILES: CCCCCCCCOC(CCC(=O)OC12CC3CC(OC(=O)CCC(OCCCCCCCC)OCCCCCCCC)(C1)CC(OC(=O)CN(CC)CC)(C3)C2)OCCCCCCCC]

A mixture of L122A-3 (0.70 g, 2.35 mmol), 4,4-bis(octyloxy)butanoic acid (2.43 g, 7.06 mmol), DCC (1.94 g, 7.06 mmol) and DMAP (288 mg, 2.35 mmol) in DCM (50 mL) was stirred at room temperature for 24 h. The reaction mixture was concentrated under reduced pressure and the residue was purified by flash chromatography (SiO2: 0-20% ethyl acetate in hexane) to afford Compound L122A as yellow oil (1.0 g, 45%); ¹H NMR (CDCl3)) δ 4.47 (t, J=5.6 Hz, 2H), 3.64-3.50 (m, 4H), 3.47-3.33 (m, 4H), 3.23 (s, 2H), 2.64 (q, J=7.2 Hz, 4H), 2.47 (p, J=12.0 Hz, 7H), 2.30 (t, J=7.6 Hz, 4H), 2.09-1.94 (m, 6H), 1.87 (q, J=7.4 Hz, 4H), 1.60-1.50 (m, 8H), 1.40-1.18 (m, 40H), 1.05 (t, J=7.2 Hz, 6H), 0.88 (t, J=6.8 Hz, 12H); CIMS m/z [M+H]950.8; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 10 min then 100% for 5 min, flow rate: 1 mL/min, column temperature: 20±2° C., detector: ELSD, t_{R}=7.7 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: A in B 60% to 100% in 10 min, flow rate: 1 mL/min, column temperature: 55±2° C., detector: CAD, t_{R}=15.2 min, purity: 99%,

### Synthetic Scheme for Compound AX-5

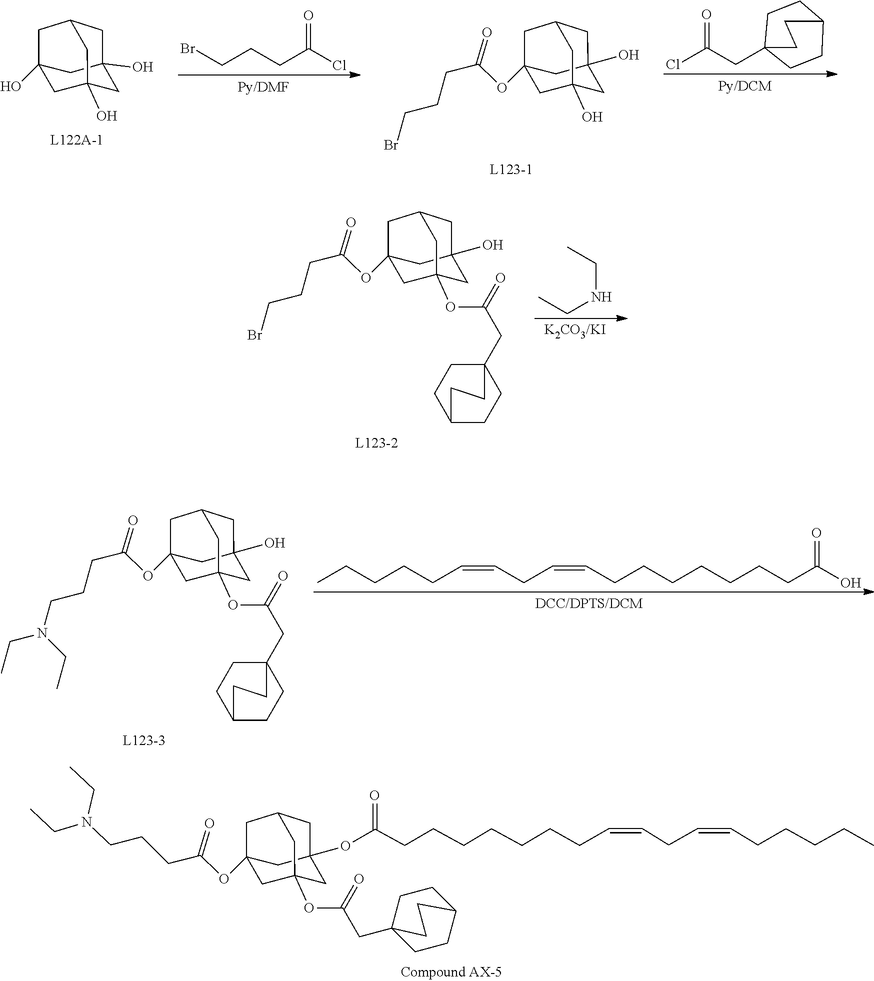

### Synthesis of (1R,3R,5S,7S)-3,5-dihydroxyadamantan-1-yl 4-bromobutanoate (L126A-1)

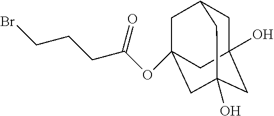 [SMILES: O=C(CCCBr)OC12CC3CC(O)(CC(O)(C3)C1)C2]

The starting material L122A-1 (3 g, 16.28 mmol) and pyridine (2 mL, 24.42 mmol) were dissolved in DMF (42 mL). 4-Bromobutanoyl chloride (1.98 mL, 16.28 mmol) was added slowly to the above solution through a syringe over 30 min at room temperature. The reaction mixture was then stirred at room temperature for 20 h. The solvent was evaporated under reduced pressure and the crude product was subjected to silica gel column using 0-10% MeOH in DCM as eluent to afford L123-1 (1.2 g, 22%) as yellow oil; ¹H-NMR (400 MHz, CDCl₃) δ 3.58 (t, 2H), 2.43 (t, 2H), 2.44-2.36 (m, 1H), 2.14-2.04 (m, 2H), 1.82-1.66 (m, 3H), 1.62 (bs, 5H), 1.56 (bs, 4H).

### Synthesis of (1R,3S,5S,7S)-3-(2-(bicyclo[2.2.2]octan-1-yl)acetoxy)-5-hydroxyadamantan-1-yl 4-bromobutanoate (L123-2)

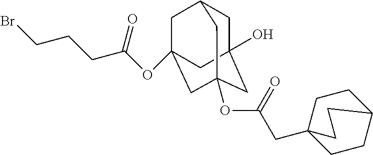 [SMILES: O=C(CCCBr)OC12CC3CC(O)(C1)CC(OC(=O)CC14CCC(CC1)CC4)(C3)C2]

2-(Bicyclo[2.2.2]octan-1-yl)acetic acid (606 mg, 3.6 mmol) was dissolved in DCM (6 mL) at 0° C. After adding DMF (12 uL), a solution of oxalyl chloride (468 uL, 5.4 mmol) in DCM (6 ml) was dropped in through syringe. After addition finished, the reaction mixture was allowed to warm to room temperature and stirred for 2 h. The solvent was evaporated under reduced pressure and the crude 2-(bicyclo[2.2.2]octan-1-yl)acetyl chloride was used directly for the next step without further purification.

The above crude acid chloride was dissolved in DCM (3 mL) and added slowly to a stirring solution of L123-1 (1.2 g, 3.6 mmol) and pyridine (438 uL, 5.4 mmol) in DCM (15 mL) over a period of one hour. After the addition finished, the reaction mixture was stirred at room temperature for 20 h. The solvent was evaporated to give the crude product which was subjected to silica gel column using 0-5% MeOH in DCM as eluent to afford L123-2 (0.9 g, 52%) as yellow oil; ¹H-NMR (400 MHz, CDCl₃) δ 3.58 (t, 2H), 2.50-2.34 (m, 5H), 2.18-2.04 (m, 2H), 1.72-1.34 (m, 25H).

### Synthesis of (1R,3S,5S,7S)-3-(2-(bicyclo[2.2.2]octan-1-yl)acetoxy)-5-hydroxyadamantan-1-yl 4-(diethylamino)butanoate (L123-3)

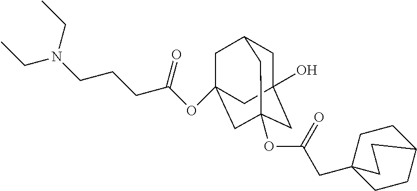 [SMILES: CCN(CC)CCCC(=O)OC12CC3CC(O)(C1)CC(OC(=O)CC14CCC(CC1)CC4)(C3)C2]

The starting material L123-2 (650 mg, 1.34 mmol) was dissolved in diethylamine (15 mL) at room temperature. An excess amount of potassium carbonate and potassium iodide were then added to the reaction mixture. The reaction was sealed and stirred at 90° C. for 20 h. Diethylamine was evaporated to provide the crude product which was purified by silica gel column using 0-20% MeOH in DCM as eluent to afford L123-3 (350 mg, 55%) as light-yellow oil; ¹H-NMR (400 MHz, CDCl₃) δ 2.50 (q, 4H), 2.44-2.28 (m, 4H), 2.23 (t, 2H), 2.18-2.02 (m, 5H), 2.00-1.82 (m, 6H), 1.72-1.58 (m, 4H), 1.58-1.28 (m, 13H), 0.99 (t, 6H); CIMS m/z [M+H]⁺ 476.3.

### Synthesis of (1S,3S,5R,7S)-3-(2-(bicyclo[2.2.2]octan-1-yl)acetoxy)-5-((4-(diethylamino) butanoyl)oxy)adamantan-1-yl(9Z,12Z)-octadeca-9,12-dienoate (Compound AX-5)

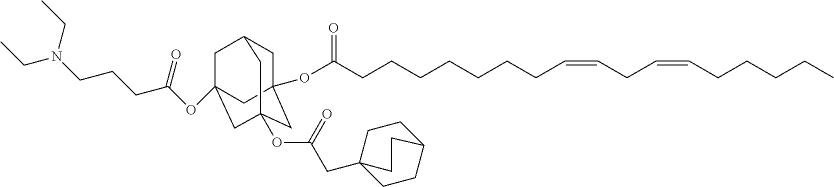 [SMILES: CCCCC/C=C\C/C=C\CCCCCCCC(=O)OC12CC3CC(OC(=O)CCCN(CC)CC)(C1)CC(OC(=O)CC14CCC(CC1)CC4)(C3)C2]

A mixture of linoleic acid (295 mg, 1.05 mmol), DCC (260 mg, 1.26 mmol) and 1,4-dimethylpyridinium 4-methylbenzenesulfonate (180 mg, 0.61 mmol) in DCM (15 mL) was stirred at room temperature for 5 min to give a clear solution. The starting material L123-3 (250 mg, 0.53 mmol) was then added to the above solution and the resulting mixture was stirred at room temperature for 3 days. The solvent was evaporated to give the crude product which was subjected to silica gel column using 0-20% MeOH in DCM with 1% NH₄OH as eluent to afford a mixture of product and impurities. This mixture was dissolved in ethyl acetate (2 mL), washed by aq. HCl (0.2 N, 2 mL×3), sat. aq. sodium bicarbonate (2 mL×2) and brine (2 mL). The organic phase was dried over anhydrous Na₂SO₄. Filtration and concentration provided Compound L123 (101 mg, 26%) as yellow oil; ¹H-NMR (400 MHz, CDCl₃) δ 5.42-5.27 (m, 4H), 2.76 (t, 2H), 2.54-2.36 (m, 13H), 2.27-2.14 (m, 4H), 2.09-1.96 (m, 10H), 1.93 (s, 2H), 1.73-1.66 (m, 2H), 1.66-1.58 (m, 2H), 1.58-1.48 (m, 7H), 1.48-1.38 (m, 6H), 1.38-1.22 (m, 14H), 0.99 (t, 6H), 0.88 (t, 3H); CIMS m/z [M+H]⁺738.5; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=7.05 min, purity: >99.9%; UPLC column: Thermo Scientific Hypersil GOLD C18, mobile phase A: acetonitrile with 0.1% trifluoroacetic acid, mobile phase B: water with 0.1% trifluoroacetic acid, use gradient: A in B 5% to 100% in 5 min, then 100% for 15 min. Flow rate: 1 mL/min, column temperature: 20±2° C., detector: CAD, t_{R}=8.02 min, purity: 89.7%.

### Synthetic Scheme for Compound AX-1

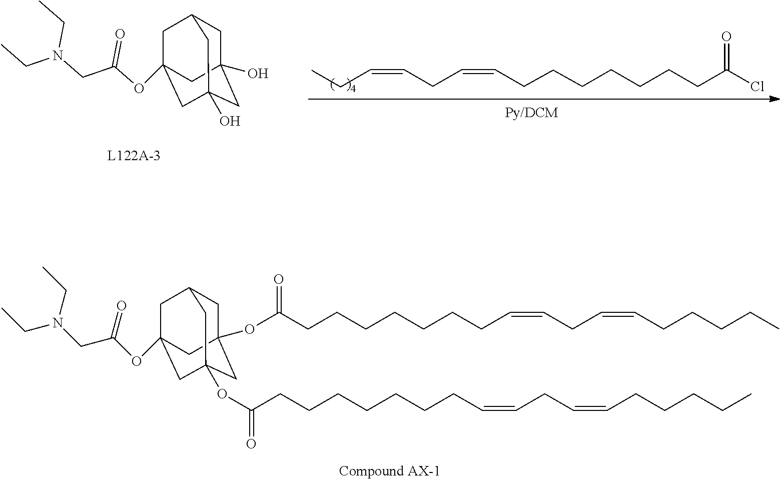

### Synthesis of (1R,3S,5R)-5-((diethylglycyl)oxy)adamantane-1,3-diyl (9Z,9′Z,12Z,12′Z)-bis(octadeca-9,12-dienoate) (Compound AX-1)

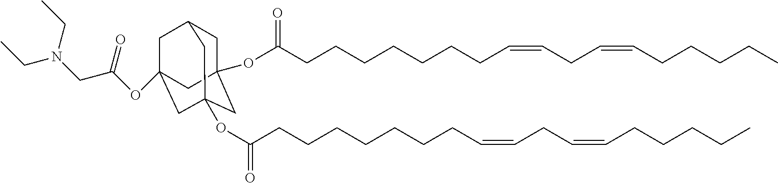 [SMILES: CCCCC/C=C\C/C=C\CCCCCCCC(=O)OC12CC3CC(OC(=O)CCCCCCC/C=C\C/C=C\CCCCC)(C1)CC(OC(=O)CN(CC)CC)(C3)C2]

To an ice bath cooled solution of linoleic acid (566 mg, 2.02 mmol) in DCM (5 mL) under nitrogen atmosphere was added oxalyl chloride (230 μl, 2.69 mmol) and DMF (50 μl) by syringe. The mixture was stirred at room temperature for 2 h and concentrated. The residue was co-evaporated with toluene (10 mL×3). The crude acid chloride was then dissolved in DCM (5 mL) and dropped into a solution of L122A-3 (100 mg, 0.34 mmol) and pyridine (160 μl, 2.02 mmol) in DCM (5 mL) at 45° C. over a period of 15 min. The resulting mixture was stirred at 45° C. for 2 h and at room temperature for 18 h. The reaction mixture was concentrated under reduced pressure and the crude was purified using flash chromatography (SiO₂: first 0-20% ethyl acetate in hexane gradient, then 0-4% methanol in dichloromethane gradient) to afford Compound AX-1 as yellow oil (69 mg, 25%); ¹HNMR (CDCl₃) δ 5.38-5.36 (m, 8H), 3.26 (s, 2H), 2.78 (t, J=5.9 Hz, 4H), 2.66 (q, J=7.2 Hz, 4H), 2.55-2.42 (m, 7H), 2.22 (t, J=7.6 Hz, 4H), 2.09-2.04 (m, 14H), 1.61-1.52 (m, 4H), 1.37-1.30 (m, 28H), 1.07 (t, J=7.2 Hz, 6H), 0.90 (t, J=6.7 Hz, 6H); CIMS m/z [M+H]822.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 10 min then 100% for 5 min, flow rate: 1 mL/min, column temperature: 20±2° C., detector: ELSD, t_{R}=7.8 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: A in B 60% to 100% in 10 min, flow rate: 1 mL/min, column temperature: 55±2° C., detector: CAD, t_{R}=13.9 min, purity: 93.13%.

### Synthetic Scheme for Compound AX-2

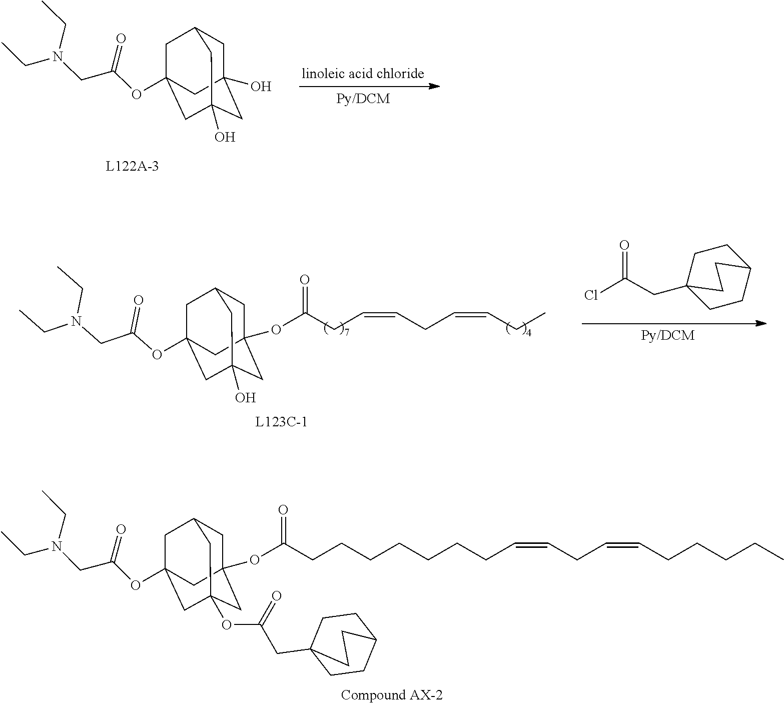

### Synthesis of (1R,3R,5S)-3-((diethylglycyl)oxy)-5-hydroxyadamantan-1-yl(9Z,12Z)-octadeca-9,12-dienoate (L123C-1)

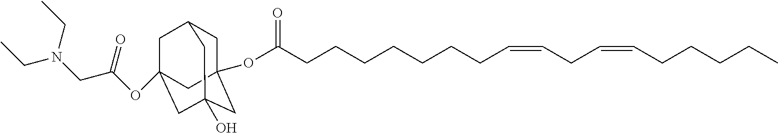 [SMILES: CCCCC/C=C\C/C=C\CCCCCCCC(=O)OC12CC3CC(O)(C1)CC(OC(=O)CN(CC)CC)(C3)C2]

To an ice bath cooled solution of linoleic acid (373 mg, 1.33 mmol) in DCM (5 mL) under nitrogen atmosphere was added oxalyl chloride (155 μl, 1.82 mmol) and DMF (50 μl) by syringe. The mixture was stirred at room temperature for 2 h and concentrated. The residue was co-evaporated with toluene (10 mL×3). The crude acid chloride was then dissolved in DCM (5 mL) and dropped into a solution of L122A-3 (180 mg, 0.61 mmol) and pyridine (120 μl, 1.51 mmol) in DCM (5 mL) at 45° C. over a period of 15 min. The resulting mixture was stirred at 45° C. for 2 h and at room temperature for 18 h. The reaction mixture was concentrated under reduced pressure and the crude was purified using flash chromatography (SiO₂: 0-2% methanol in dichloromethane gradient) to afford L123C-1 as yellowish oil (144 mg, 43%); ¹HNMR (CDCl₃) δ 4.82-5.62 (4H), 3.24 (s, 2H), 2.77 (t, J=5.6 Hz, 2H), 2.68-2.62 (q, J=7.2 Hz, 4H), 2.47-2.39 (m, 3H), 2.23-2.17 (m, 2H), 2.13-1.98 (m, 10H), 1.64-1.52 (m, 6H), 1.37-1.20 (m, 14H), 1.05 (t, J=7.2 Hz, 6H), 0.88 (t, J=6.9 Hz, 3H); CIMS m/z [M+H]560.4.

### Synthesis of (1R,3S,5S)-3-(2-(bicyclo[2.2.2]octan-1-yl)acetoxy)-5-((diethylglycyl)oxy) adamantan-1-yl(9Z,12Z)-octadeca-9,12-dienoate (Compound AX-2)

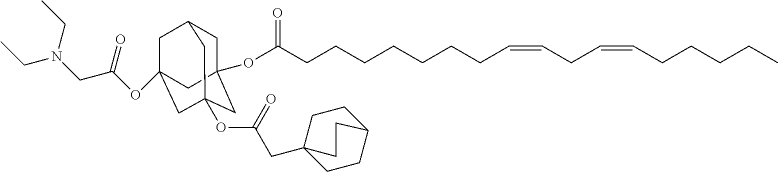 [SMILES: CCCCC/C=C\C/C=C\CCCCCCCC(=O)OC12CC3CC(OC(=O)CN(CC)CC)(C1)CC(OC(=O)CC14CCC(CC1)CC4)(C3)C2]

To an ice bath cooled solution of 2-(bicyclo[2.2.2]octan-1-yl)acetic acid (195 mg, 1.16 mmol) in DCM (5 mL) under nitrogen atmosphere was added oxalyl chloride (208 μl, 2.32 mmol) and DMF (50 μl) by syringe. The mixture was stirred at room temperature for 2 h and concentrated. The residue was co-evaporated with toluene (10 mL×3). The crude acid chloride was then dissolved in DCM (5 mL) and dropped into a solution of L123C-1 (130 mg, 0.23 mmol) and pyridine (184 μl, 2.32 mmol) in DCM (5 mL) at 45° C. over a period of 15 min. The resulting mixture was stirred at 45° C. for 2 h and at room temperature for 18 h. The reaction mixture was concentrated under reduced pressure and the crude was purified using flash chromatography (SiO₂: 0-2% methanol in dichloromethane gradient) to afford Compound AX-2 as yellow oil (63 mg, 38%); ¹HNMR (CDCl₃) δ 5.39-5.30 (m, 4H), 3.24 (s, 2H), 2.77 (t, J=6.1 Hz, 2H), 2.64 (q, J=7.2 Hz, 4H), 2.54-2.41 (m, 7H), 2.20 (t, J=7.4 Hz, 2H), 2.08-2.03 (m, 10H), 1.94 (s, 2H), 1.57-1.25 (m, 29H), 1.05 (t, J=7.2 Hz, 6H), 0.88 (t, J=6.9 Hz, 3H); CIMS m/z [M+H]710.4; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 10 min then 100% for 5 min, flow rate: 1 mL/min, column temperature: 20±2° C., detector: ELSD, t_{R}=9.7 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: A in B 60% to 100% in 10 min, flow rate: 1 mL/min, column temperature: 55±2° C., detector: CAD, t_{R}=11.4 min, purity: 94.22%.

### Synthetic Scheme for Compound AX-8

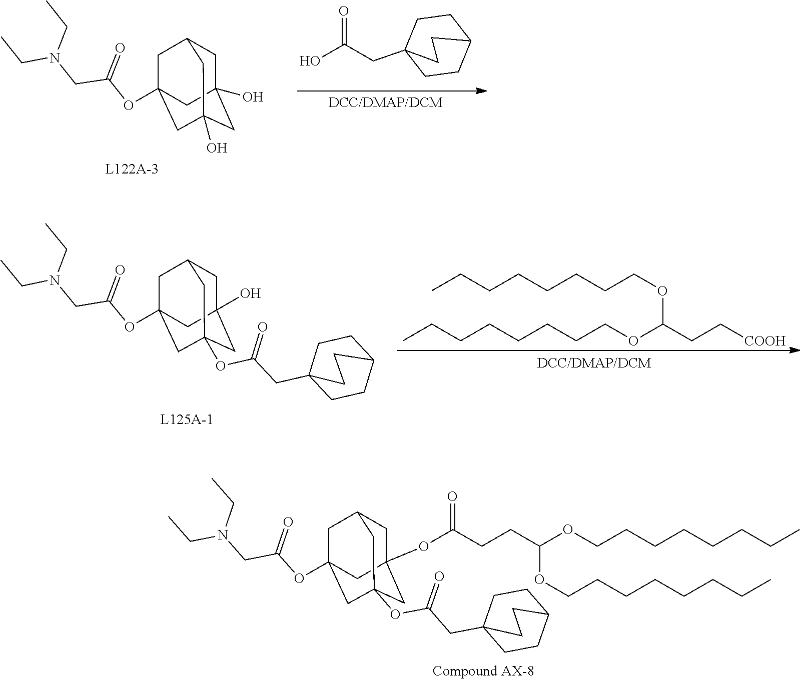

### Synthesis of (1S,3S,5R)-3-(2-(bicyclo[2.2.2]octan-1-yl)acetoxy)-5-hydroxyadamantan-1-yl diethylglycinate (L125A-1)

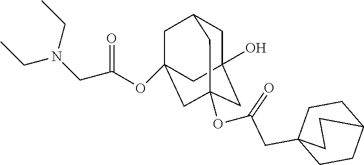 [SMILES: CCN(CC)CC(=O)OC12CC3CC(O)(C1)CC(OC(=O)CC14CCC(CC1)CC4)(C3)C2]

A mixture of L122A-3 (300 mg, 1.01 mmol), 2-(bicyclo[2.2.2]octan-1-yl)acetic acid (170 mg, 1.01 mmol), DCC (416 mg, 2.02 mmol) and DMAP (123 mg, 1.01 mmol) in DCM (10 mL) was stirred at room temperature for 24 h. The reaction mixture was filtered over Celite. The filtrate was concentrated under reduced pressure and the residue was purified using flash chromatography (SiO₂: 0-5% methanol in dichloromethane) to give impure product which was repurified using flash chromatography (SiO₂, 0-100% ethyl acetate in hexanes gradient) to afford L125A-1 as yellow oil (140 mg, 31%); ¹HNMR (CDCl₃) δ 3.22 (s, 2H), 2.63 (q, J=7.2 Hz, 4H), 2.42-2.38 (m, 3H), 2.15-2.09 (m, 4H), 2.03-1.97 (m, 4H), 1.94 (s, 2H), 1.64 (d, J=2.7 Hz, 2H), 1.56-1.49 (m, 10H), 1.45-1.41 (m, 4H), 1.04 (t, J=7.1 Hz, 6H); CIMS m/z [M+H]448.3.

### Synthesis of (1R,3S,5R)-3-(2-(bicyclo[2.2.2]octan-1-yl)acetoxy)-5-((diethylglycyl)oxy) adamantan-1-yl 4,4-bis(octyloxy)butanoate) (Compound AX-8)

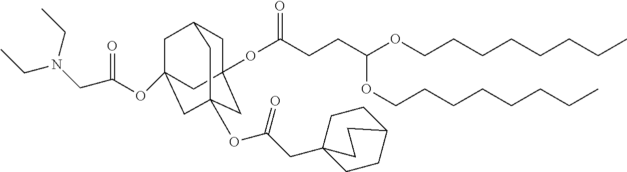 [SMILES: CCCCCCCCOC(CCC(=O)OC12CC3CC(OC(=O)CN(CC)CC)(C1)CC(OC(=O)CC14CCC(CC1)CC4)(C3)C2)OCCCCCCCC]

A mixture of L125A-1 (140 mg, 0.31 mmol), 4,4-bis(octyloxy)butanoic acid (323 mg, 0.94 mmol), DCC (387 mg, 1.88 mmol) and DMAP (38 mg, 0.31 mmol) in DCM (10 mL) was stirred at room temperature for 24 h. The reaction mixture was filtered through Celite. The filtrate was concentrated under reduced pressure and the residue was purified using flash chromatography (SiO₂: 0-50% ethyl acetate in hexanes gradient) to afford Compound AX-8 as yellow oil (145 mg, 58%); ¹HNMR (CDCl₃) δ 4.47 (t, J=5.5 Hz, 1H), 3.56 (dd, J=15.7, 7.0 Hz, 2H), 3.40 (dd, J=15.6, 6.9 Hz, 2H), 3.23 (s, 2H), 2.64 (q, J=7.1 Hz, 4H), 2.54-2.41 (m, 7H), 2.31 (t, J=7.4 Hz, 2H), 2.06-2.02 (m, 6H), 1.95 (s, 2H), 1.87 (dd, J=13.3, 7.1 Hz, 2H), 1.61-1.50 (m, 14H) 1.45 (d, J=9.2 Hz, 4H), 1.28 (s, 20H), 1.05 (t, J=7.1 Hz, 6H), 0.90-0.87 (m, 6H); CIMS m/z [M+H]774.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 10 min then 100% for 5 min, flow rate: 1 mL/min, column temperature: 20±2° C., detector: ELSD, t_{R}=7.2 min, purity: 99.8%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: A in B 60% to 100% in 10 min, flow rate: 1 mL/min, column temperature: 55±2° C., detector: CAD, t_{R}=11.8 min, purity: >96.55 Synthetic Scheme for Compound AX-4

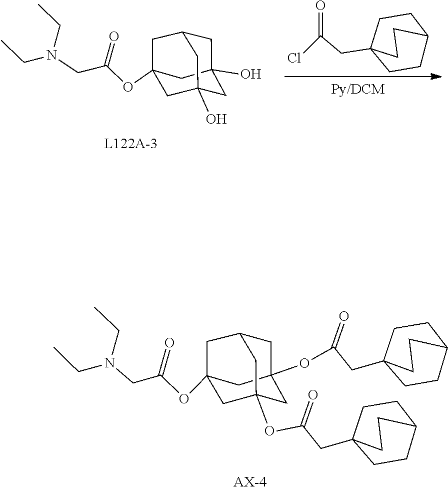

### Synthesis of (1R,3S,5R)-5-((diethylglycyl)oxy)adamantane-1,3-diyl bis(2-(bicyclo[2.2.2]octan-1-yl)acetate) (L125A-2)

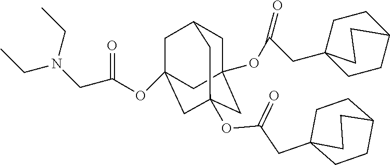 [SMILES: CCN(CC)CC(=O)OC12CC3CC(OC(=O)CC45CCC(CC4)CC5)(C1)CC(OC(=O)CC14CCC(CC1)CC4)(C3)C2]

To an ice bath cooled solution of 2-(bicyclo[2.2.2]octan-1-yl)acetic acid acid (300 mg, 1.78 mmol) in DCM (5 mL) under nitrogen atmosphere was added oxalyl chloride (623 μl, 7.26 mmol) and DMF (50 μl) by syringe. The mixture was stirred at room temperature for 2 h and concentrated. The residue was co-evaporated with toluene (10 mL×3). The crude acid chloride was then dissolved in DCM (5 mL) and dropped into a solution of L122A-3 (360 mg, 1.21 mmol) and pyridine (586 μl, 7.26 mmol) in DCM (5 mL) at 45° C. over a period of 15 min. The resulting mixture was stirred at 45° C. for 2 h and at room temperature for 18 h. The reaction mixture was concentrated under reduced pressure and the crude was purified using flash chromatography (SiO₂: 0-30% ethyl acetate in hexanes gradient) to afford AX-4 as yellow oil (64 mg, 9%); ¹HNMR (CDCl₃) δ 3.22 (s, 2H), 2.63 (q, J=7.0 Hz, 4H), 2.53-2.39 (m, 7H), 2.09-2.03 (m, 6H), 1.93 (s, 4H), 1.44 (s, 26H), 1.04 (t, J=7.1 Hz, 6H); CIMS m/z [M+H]598.4; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 10 min then 100% for 5 min, flow rate: 1 mL/min, column temperature: 20±2° C., detector: ELSD, t_{R}=3.9 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: A in B 60% to 100% in 10 min, flow rate: 1 mL/min, column temperature: 55±2° C., detector: CAD, t_{R} 6.2 min, purity: 93.09%.

### Synthetic Scheme for Compound AX-6

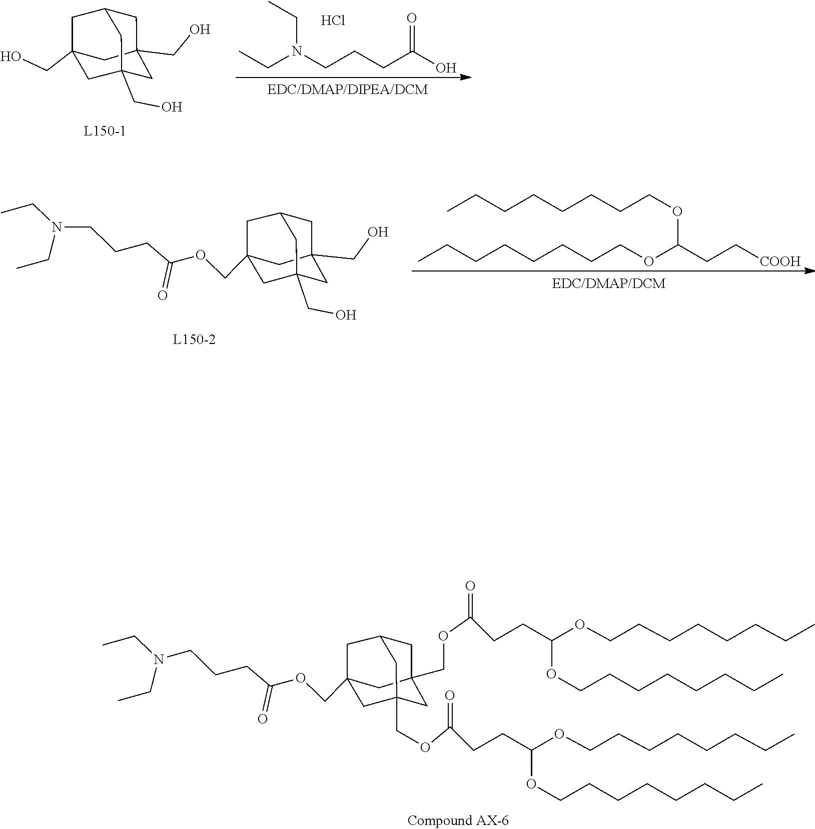 [SMILES: OCC12CC3CC(CO)(C1)CC(CO)(C3)C2 ; CCN(CC)CCCC(=O)O ; Cl ; CCN(CC)CCCC(=O)OCC12CC3CC(CO)(CC(CO)(C3)C1)C2 ; CCCCCCCCOC(CCC(=O)O)OCCCCCCCC ; CCCCCCCCOC(CCC(=O)OCC12CC3CC(COC(=O)CCCN(CC)CC)(C1)CC(COC(=O)CCC(OCCCCCCCC)OCCCCCCCC)(C3)C2)OCCCCCCCC]

### Synthesis of ((1S,3R,5S,7S)-3,5-bis(hydroxymethyl)adamantan-1-yl)methyl 4-(diethylamino) butanoate (L150-2)

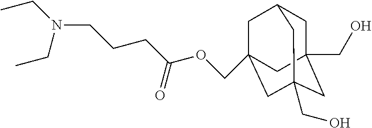 [SMILES: CCN(CC)CCCC(=O)OCC12CC3CC(CO)(CC(CO)(C3)C1)C2]

A solution of 4-(diethylamino)butanoic acid hydrochloride (0.64 g, 3.3 mmol), EDC (5.0 g, 26.5 mmol), DIPEA (0.5 mL) and DMAP (813 mg, 6.6 mmol) in DCM (20 mL) was stirred at room temperature for 30 min. The starting alcohol L150-1 (1.5 g, 6.6 mmol) was added in. The reaction mixture was stirred at room temperature for one hour and concentrated under reduced pressure. The residue was purified by column chromatography (SiO₂, solvent 0-10% MeOH/DCM) to provide L150-2 (1.3 g, 53%) as colorless liquid; ¹H NMR (CDCl₃): 0.98 (t, 6H), 1.38-1.34 (m, 6H), 1.45-1.40 (m, 6H), 1.78-1.73 (m, 2H), 2.18-2.16 (m, 3H), 2.51-246 (m, 4H), 2.40 (t, 2H), 2.31 (t, 2H), 3.26-3.23 (m, 4H), 3.74-3.72 (m, 2H); CIMS m/z [M+H]⁺ 368.1.

### Synthesis of ((1R,3S,5R,7R)-5-(((4-(diethylamino)butanoyl)oxy)methyl)adamantane-1,3-diyl)bis(methylene) bis(4,4-bis(octyloxy)butanoate) (Compound AX-6)

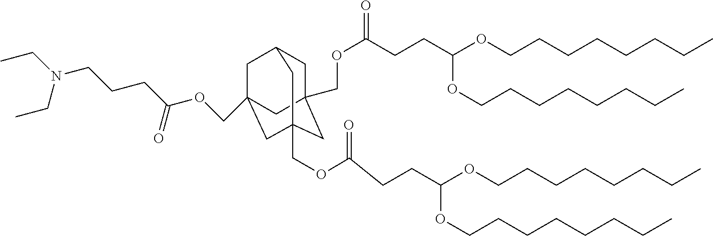 [SMILES: CCCCCCCCOC(CCC(=O)OCC12CC3CC(COC(=O)CCCN(CC)CC)(C1)CC(COC(=O)CCC(OCCCCCCCC)OCCCCCCCC)(C3)C2)OCCCCCCCC]

A solution of 4,4-bis(octyloxy)butanoic acid (1.8 g, 5.2 mmol), EDC (1.6 g, 8.7 mmol) and DMAP (267 mg, 2.1 mmol) in DCM (15 mL) was stirred at room temperature for 30 min. The starting alcohol L150-2 (800 mg, 2.1 mmol) was added in. The reaction mixture was stirred at room temperature for 12 h and concentrated under reduced pressure. The residue was purified by column chromatography (SiO₂, solvent 0-10% MeOH/DCM) to provide Compound AX-6 (1.2 g, 55%) as colorless liquid; ¹H NMR (CDCl₃): 0.85 (t, 12H), 1.11 (t, 6H), 1.4-1.25 (m, 46H), 1.45-1.44 (m, 6H), 1.61-1.50 (m, 8H), 1.93-1.86 (m, 6H), 2.22-2.10 (s, 1H), 2.40-2.35 (m, 6H), 2.69-2.58 (m, 6H), 3.41-3.33 (m, 4H), 3.57-3.51 (m, 4H), 3.72 (s, 6H), 4.47 (t, 2H); CIMS m/z [M+H]⁺ 1020.8. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 100% in 17 min, flow rate: 1 mL/min, column temperature: 20±2° C., detector: ELSD, t_{R}=7.93 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: B in A 60% to 100% in 17 min, flow rate: 0.5 mL/min, column temperature: 20±2° C., detector: CAD, t_{R}=13.50 min, purity: >99%.

### Synthetic Scheme for Compound AX-7

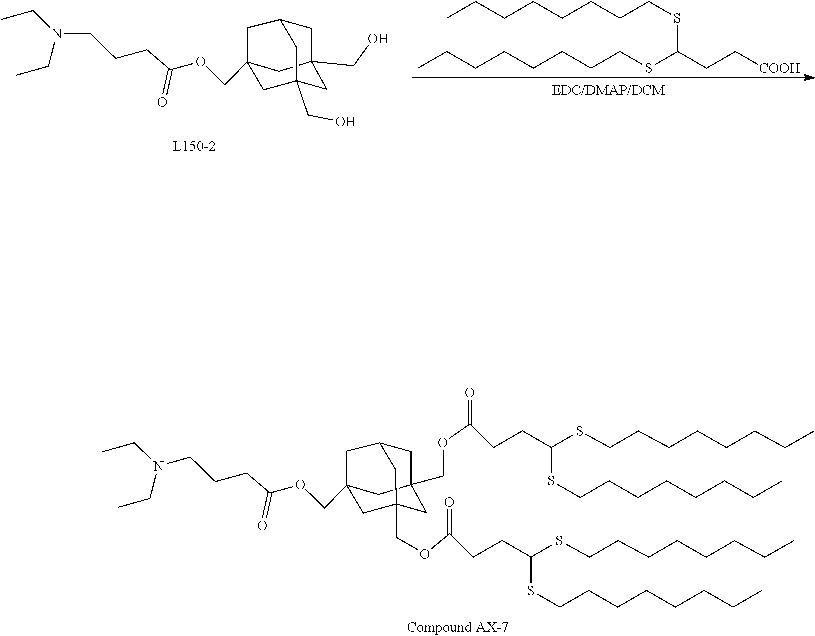 [SMILES: CCN(CC)CCCC(=O)OCC12CC3CC(CO)(CC(CO)(C3)C1)C2 ; CCCCCCCCSC(CCC(=O)O)SCCCCCCCC ; CCCCCCCCSC(CCC(=O)OCC12CC3CC(COC(=O)CCCN(CC)CC)(C1)CC(COC(=O)CCC(SCCCCCCCC)SCCCCCCCC)(C3)C2)SCCCCCCCC]

### Synthesis of ((1R,3S,5R,7R)-5-(((4-(diethylamino)butanoyl)oxy)methyl)adamantane-1,3-diyl)bis(methylene) bis(4,4-bis(octylthio)butanoate) (Compound AX-7)

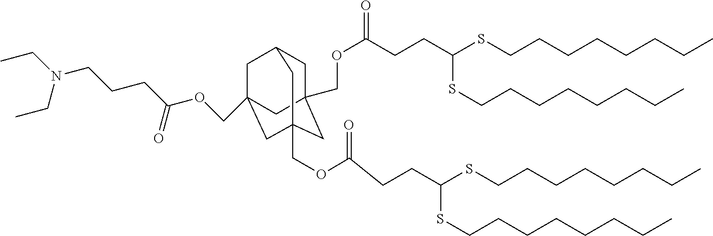 [SMILES: CCCCCCCCSC(CCC(=O)OCC12CC3CC(COC(=O)CCCN(CC)CC)(C1)CC(COC(=O)CCC(SCCCCCCCC)SCCCCCCCC)(C3)C2)SCCCCCCCC]

A solution of 4,4-bis(octylthio)butanoic acid (184 mg, 0.49 mmol), EDC (156 mg, 0.81 mmol) and DMAP (25 mg, 0.2 mmol) in DCM (5 mL) was stirred at room temperature for 20 min. The starting alcohol L150-2 (75 mg, 0.2 mmol) was added in. The reaction mixture was stirred at room temperature for 12 h and concentrated under reduced pressure. The residue was purified by column chromatography (SiO₂, solvent 0-10% MeOH/DCM) to provide Compound AX-7 (125 mg, 56%) as colorless liquid; ¹H NMR (CDCl₃): 0.85 (t, 12H), 1.16 (t, 6H), 1.36-1.20 (m, 46H), 1.43-1.42 (m, 6H), 1.57-1.53 (m, 6H), 1.92-1.88 (m, 2H), 2.10-2.05 (m, 4H), 2.16-2.19 (m, 1H), 2.39 (t, 2H), 2.52-2.63 (m, 14H), 2.66-2.88 (m, 6H), 3.74 (s, 6H), 3.79 (t, 2H); CIMS m/z [M+H]⁺ 1084.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 100% in 17 min, flow rate: 1 mL/min, column temperature: 20±2° C., detector: ELSD, t_{R}=8.35 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: B in A 60% to 100% in 17 min, flow rate: 0.5 mL/min, column temperature: 20±2° C., detector: CAD, t_{R}=13.58 min, purity: 85.57%.

### Synthetic Scheme for Compound AX-9

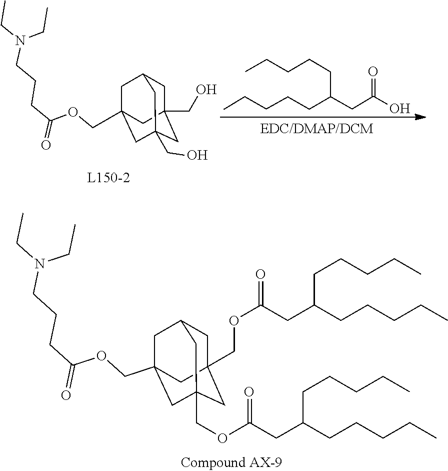

### Synthesis of ((1R,3S,5R,7R)-5-(((4-(diethylamino)butanoyl)oxy)methyl)adamantane-1,3-diyl)bis(methylene) bis(3-pentyloctanoate)) (Compound AX-9)

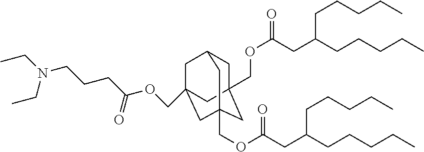 [SMILES: CCCCCC(CCCCC)CC(=O)OCC12CC3CC(COC(=O)CCCN(CC)CC)(C1)CC(COC(=O)CC(CCCCC)CCCCC)(C3)C2]

A solution of 3-pentyloctanoic acid (10⁵ mg, 0.49 mmol), EDC (156 mg, 0.81 mmol) and DMAP (25 mg, 0.2 mmol) in DCM (5 mL) was stirred at room temperature for 30 min. The starting alcohol L150-2 (75 mg, 0.2 mmol) was added in. The reaction mixture was stirred at room temperature for 12 h and concentrated under reduced pressure. The residue was purified by column chromatography (SiO₂, solvent 0-10% MeOH/DCM) to provide Compound AX-9 (88 mg, 66%) as colorless liquid; ¹H NMR (CDCl₃): 0.85 (t, 12H), 1.33-1.27 (m, 44H), 1.48-1.42 (m, 6H), 1.86-1.79 (m, 2H), 2.07-1.97 (m, 2H), 2.18-2.14 (m, 1H), 2.24-2.22 (m, 4H), 2.42 (t, 2H), 2.98-2.86 (m, 6H), 3.73-3.71 (m, 6H); CIMS m/z [M+H]⁺ 760.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 100% in 17 min, flow rate: 1 mL/min, column temperature: 20±2° C., detector: ELSD, t_{R}=6.59 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: B in A 60% to 100% in 17 min, flow rate: 0.5 mL/min, column temperature: 20±2° C., detector: CAD, t_{R}=11.88 min, purity: 92.45%.

### Synthetic Scheme for Compound AX-10

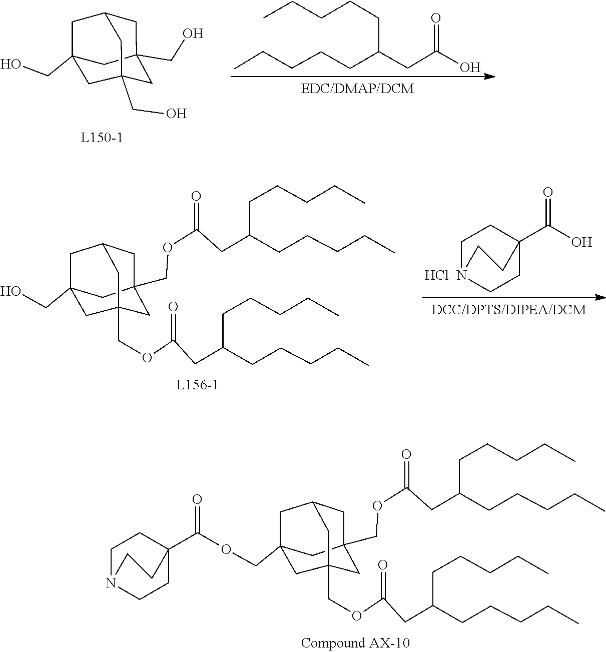

### Synthesis of ((1R,3S,5R,7R)-5-(hydroxymethyl)adamantane-1,3-diyl)bis(methylene) bis(3-pentyloctanoate) (L156-1)

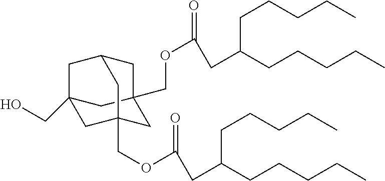 [SMILES: CCCCCC(CCCCC)CC(=O)OCC12CC3CC(CO)(C1)CC(COC(=O)CC(CCCCC)CCCCC)(C3)C2]

A solution of 3-pentyloctanoic acid (428 mg, 2.0 mmol), EDC (768 mg, 4.0 mmol) and DMAP (122 mg, 1.0 mmol) in DCM (6 mL) was stirred at room temperature for 30 min, and then added dropwise into a solution of the starting alcohol L150-1 (226 mg, 1.0 mmol) in DMF (4 mL) over a period of 30 min. The resulting reaction mixture was stirred at room temperature for 20 h and concentrated under reduced pressure. The residue was purified by column chromatography (SiO₂, 0-100% EtOAc in hexane) to provide L156-1 (253 mg, 41%) as colorless liquid; ¹H-NMR (300 MHz, CDCl₃) δ 3.72 (s, 4H), 3.28 (s, 2H), 2.23 (d, J=6.8 Hz, 4H), 2.19 (m, 1H), 1.91-1.75 (m, 2H), 1.41-1.30 (m, 6H), 1.29-1.12 (m, 38H), 0.87 (t, J=7.2 Hz, 12H).

### Synthesis of ((1R,3S,5S,7S)-5-(((quinuclidine-4-carbonyl)oxy)methyl)adamantane-1,3-diyl)bis(methylene) bis(3-pentyloctanoate) (Compound AX-10)

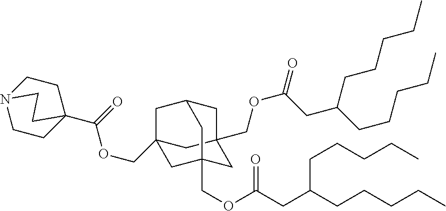 [SMILES: CCCCCC(CCCCC)CC(=O)OCC12CC3CC(COC(=O)CC(CCCCC)CCCCC)(C1)CC(COC(=O)C14CCN(CC1)CC4)(C3)C2]

A mixture of quinuclidine-4-carboxylic acid hydrochloride (115 mg, 0.6 mmol), DCC (205 mg, 0.8 mmol), 1,4-dimethylpyridinium 4-methylbenzenesulfonate (120 mg, 0.44 mmol) and DIPEA (0.5 mL) in DCM (10 mL) was stirred at room temperature for 15 min. A solution of L156-1 (250 mg, 0.4 mmol) in DCM (2 mL) was then added in and the resulting mixture was stirred at room temperature for 3 days. The solvent was evaporated to give the crude product which was subjected to silica gel column using 0-20% MeOH in DCM with 1% NH₄OH as eluent to afford Compound AX-10 (60 mg, 20%) as slightly yellow oil; ¹H-NMR (300 MHz, CDCl₃) δ 3.80 (s, 2H), 3.74 (s, 4H), 3.36-3.32 (m, 6H), 2.23 (d, J=6.8 Hz, 4H), 2.21 (m, 1H), 2.18-2.05 (m, 6H), 1.90-1.76 (m, 2H), 1.51-1.40 (m, 6H), 1.32-1.15 (m, 38H), 0.87 (t, J=7.2 Hz, 12H); APCI-MS: m/z [M+H]⁺ 756.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: t_{R}=7.82 min, purity: 96.24%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 40% to 100% in 5 min, then 100% for 15 min. Flow rate: 0.5 mL/min, column temperature: 20±2° C., detector: CAD, t_{R}=12.38 min, purity: 92.85%.

### Synthetic Scheme for Compound AX-11

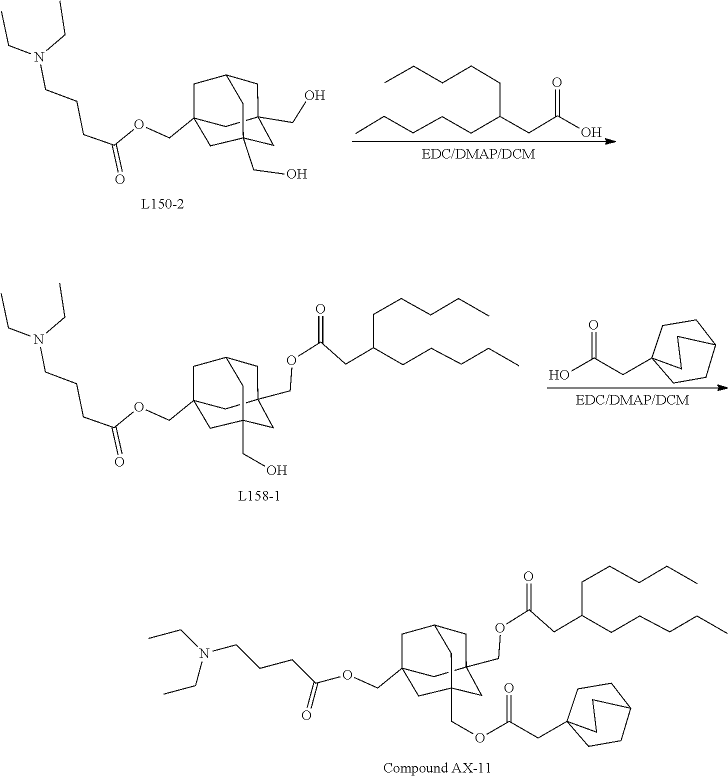

### Synthesis of ((1R,3S,5R,7R)-3-(((4-(diethylamino)butanoyl)oxy)methyl)-5-(hydroxymethyl) adamantan-1-yl)methyl 3-pentyloctanoate (L158-1)

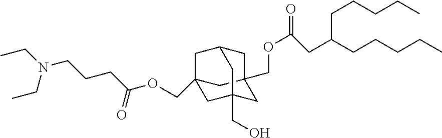 [SMILES: CCCCCC(CCCCC)CC(=O)OCC12CC3CC(CO)(CC(COC(=O)CCCN(CC)CC)(C3)C1)C2]

A solution of 3-pentyloctanoic acid (121 mg, 0.56 mmol), EDC (542 mg, 2.8 mmol) and DMAP (86 mg, 0.70 mmol) in DCM (5 mL) was stirred at room temperature for 20 min. The starting alcohol L150-2 (260 mg, 0.70 mmol) was added in. The reaction mixture was stirred at room temperature for 12 h and concentrated under reduced pressure. The residue was purified by column chromatography (SiO₂, solvent 0-10% MeOH/DCM) to provide L158-1 (85 mg, 22%) as colorless liquid; ¹H NMR (CDCl₃): 0.86, (t, 6H), 1.16 (t, 6H), 1.26-1.24 (m, 21H), 1.44-1.35 (m, 7H), 1.92-1.81 (m, 3H), 2.24-2.22 (m, 3H), 2.38-2.35 (m, 2H), 2.75-2.67 (m, 5H), 3.01 (s, 2H), 3.27 (s, 2H), 3.74-3.72 (m, 4H); CIMS m/z [M+H]⁺ 564.5.

### Synthesis of ((1S,3R,5R,7R)-3-((2-(bicyclo[2.2.2]octan-1-yl)acetoxy)methyl)-5-(((4-(diethylamino)butanoyl)oxy)methyl)adamantan-1-yl)methyl 3-pentyloctanoate (Compound AX-11)

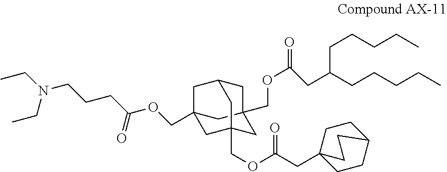 [SMILES: CCCCCC(CCCCC)CC(=O)OCC12CC3CC(COC(=O)CCCN(CC)CC)(C1)CC(COC(=O)CC14CCC(CC1)CC4)(C3)C2]

A solution of 2-(bicyclo[2.2.2]octan-1-yl)acetic acid (38 mg, 0.22 mmol), EDC (115 mg, 0.60 mmol) and DMAP (18 mg, 0.15 mmol) in DCM (5 mL) was stirred at room temperature for 20 min. The starting alcohol L158-1 (85 mg, 0.15 mmol) was added in. The reaction mixture was stirred at room temperature for 12 h and concentrated under reduced pressure. The residue was purified by column chromatography (SiO₂, solvent 0-10% MeOH/DCM) to provide Compound AX-11 (78 mg, 74%) as colorless liquid; ¹H NMR (CDCl₃): 0.85 (t, 6H), 1.28-1.19 (m, 27H), 1.53-1.43 (m, 20H), 1.85-1.78 (m, 1H), 2.03-1.97 (m, 4H), 2.23-2.19 (m, 3H), 2.42 (t, 2H), 2.97-2.85 (m, 6H), 3.72-3.67 (m, 6H); CIMS m/z [M+H]⁺ 714.6. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 100% in 17 min, flow rate: 1 mL/min, column temperature: 20±2° C., detector: ELSD, t_{R}=6.70 min, purity: >99%; UPLC column: Waters Aquity UPLC® CSH™, C18, 1.7 μm, 3.0×150 mm, (Part No. 186005302), mobile phase A: water with 0.1% trifluoroacetic acid, mobile phase B: acetonitrile with 0.1% trifluoroacetic acid, use gradient: B in A 60% to 100% in 17 min, flow rate: 0.5 mL/min, column temperature: 20±2° C., detector: CAD, t_{R}=10.10 min, purity: 94.04%.

### Synthetic Scheme for Compound AX-12

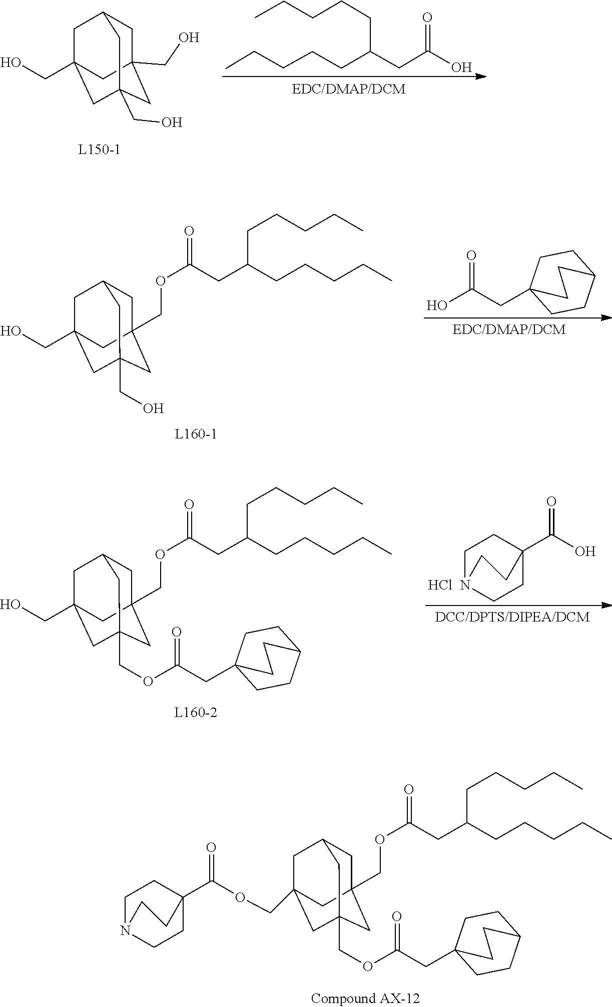

### Synthesis of ((1S,3R,5S,7S)-3,5-bis(hydroxymethyl)adamantan-1-yl)methyl 3-pentyloctanoate (L160-1)

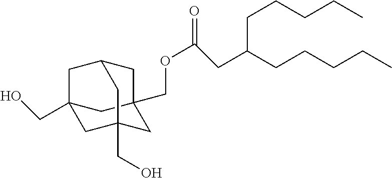 [SMILES: CCCCCC(CCCCC)CC(=O)OCC12CC3CC(CO)(CC(CO)(C3)C1)C2]

A solution of 3-pentyloctanoic acid (428 mg, 2.0 mmol), EDC (768 mg, 4.0 mmol) and DMAP (122 mg, 1.0 mmol) in DCM (12 mL) was stirred at room temperature for 30 min, and then added dropwise into a solution of the starting alcohol L150-1 (452 mg, 2.0 mmol) in DMF (6 mL) over a period of 30 min. The resulting reaction mixture was stirred at room temperature for 18 h and concentrated under reduced pressure. The residue was purified by column chromatography (SiO₂, 0-100% EtOAc in hexane) to provide L160-1 (330 mg, 70%) as colorless liquid; ¹H-NMR (300 MHz, CDCl₃) δ 3.74 (s, 4H), 3.29-3.28 (m, 2H), 2.23 (d, J=6.8 Hz, 2H), 2.22-2.18 (m, 1H), 1.91-1.76 (m, 1H), 1.49-1.40 (m, 6H), 1.35-1.15 (m, 22H), 0.87 (t, J=7.2 Hz, 6H).

### Synthesis of ((1S,3R,5R,7R)-3-((2-(bicyclo[2.2.2]octan-1-yl)acetoxy)methyl)-5-(hydroxymethyl)adamantan-1-yl)methyl 3-pentyloctanoate (L160-2)

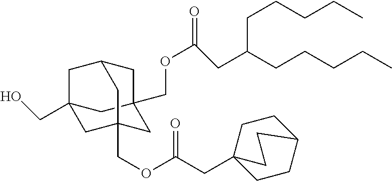 [SMILES: CCCCCC(CCCCC)CC(=O)OCC12CC3CC(CO)(C1)CC(COC(=O)CC14CCC(CC1)CC4)(C3)C2]

A solution of 2-(bicyclo[2.2.2]octan-1-yl)acetic acid (125 mg, 0.74 mmol), EDC (320 mg, 1.66 mmol) and DMAP (52 mg, 0.41 mmol) in DCM (12 mL) was stirred at room temperature for 30 min, and then added dropwise into a solution of the starting alcohol L160-1 (350 mg, 0.83 mmol) in DMF (4 mL) over a period of 30 min. The reaction mixture was stirred at room temperature for 20 h and concentrated under reduced pressure. The residue was purified by column chromatography (SiO₂, 0-100% EtOAc in hexane) to provide L160-2 (181 mg, 50%) as colorless liquid; ¹H-NMR (300 MHz, CDCl₃) δ 3.73 (s, 2H), 3.69 (s, 2H), 3.31-3.28 (m, 2H), 2.23 (d, J=6.8 Hz, 2H), 2.22-2.18 (m, 1H), 2.05 (s, 2H), 1.88-1.76 (m, 1H), 1.60-1.50 (m, 13H), 1.49-1.39 (m, 6H), 1.38-1.15 (m, 22H), 0.87 (t, J=7.2 Hz, 6H).

### Synthesis of ((1R,3S,5R,7R)-3-((2-(bicyclo[2.2.2]octan-1-yl)acetoxy)methyl)-5-(((3-pentyloctanoyl)oxy)methyl)adamantan-1-yl)methyl quinuclidine-4-carboxylate (Compound AX-12)

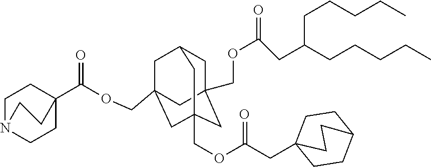 [SMILES: CCCCCC(CCCCC)CC(=O)OCC12CC3CC(COC(=O)CC45CCC(CC4)CC5)(C1)CC(COC(=O)C14CCN(CC1)CC4)(C3)C2]

A mixture of quinuclidine-4-carboxylic acid hydrochloride (130 mg, 0.67 mmol), DCC (232 mg, 0.9 mmol), 1,4-dimethylpyridinium 4-methylbenzenesulfonate (135 mg, 0.49 mmol) and DIPEA (0.5 mL) in DCM (10 mL) was stirred at room temperature for 15 min. A solution of L160-2 (260 mg, 0.45 mmol) in DCM (2 mL) was then added and the resulting mixture was stirred at room temperature for 3 days. The solvent was evaporated to give the crude product which was subjected to silica gel column using 0-20% MeOH in DCM with 1% NH₄OH as eluent to afford Compound AX-12 (156 mg, 48%) as slightly yellow oil; ¹H-NMR (300 MHz, CDCl₃) δ 3.73 (s, 4H), 3.69 (s, 2H), 2.92-2.87 (m, 6H), 2.23 (d, J=6.8 Hz, 2H), 2.21-2.18 (m, 1H), 2.03 (s, 2H), 1.85-1.76 (m, 1H), 1.73-1.65 (m, 6H), 1.60-1.49 (m, 13H), 1.50-1.39 (m, 6H), 1.38-1.16 (m, 22H), 0.85 (t, J=7.0 Hz, 6H); APCI-MS: m/z [M+H]⁺ 710.5; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: t_{R}=6.76 min, purity: >99%; UPLC column: Waters Aquity UPLC® CSH™, C18, 1.7 μm, 3.0×150 mm, (Part No. 186005302)), mobilephase A: acetonitrile with 0.1% trifluoroacetic acid, mobile phase B: water with 0.1% trifluoroacetic acid, use gradient: A in B 40% to 100% in 5 min, then 100% for 15 min. Flow rate: 0.5 mL/min, column temperature: 20±2° C., detector: CAD, t_{R}=8.30 min, purity: 96.29%.

### Example 5: oCAR Expression, Percent Cytotoxicity Killing, and Frequency of Anti-CD19 CARs in Human and T Cells

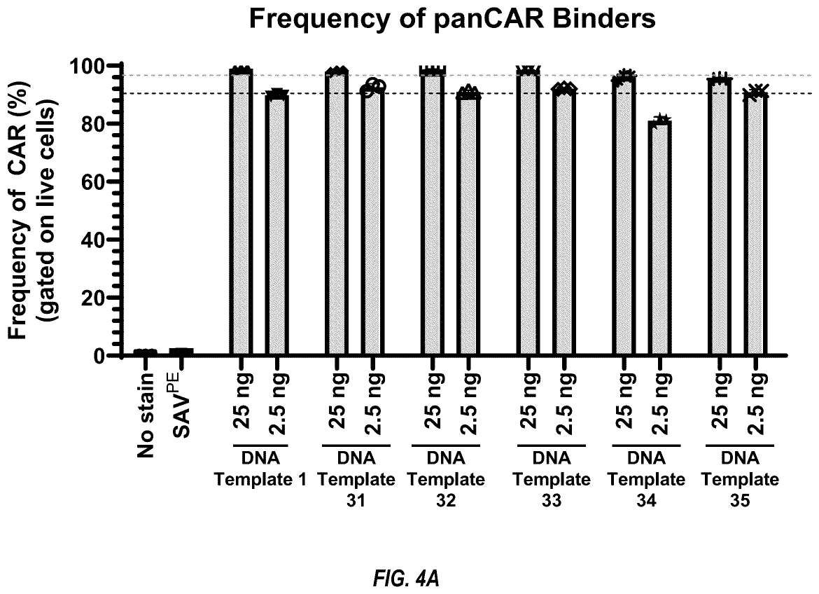

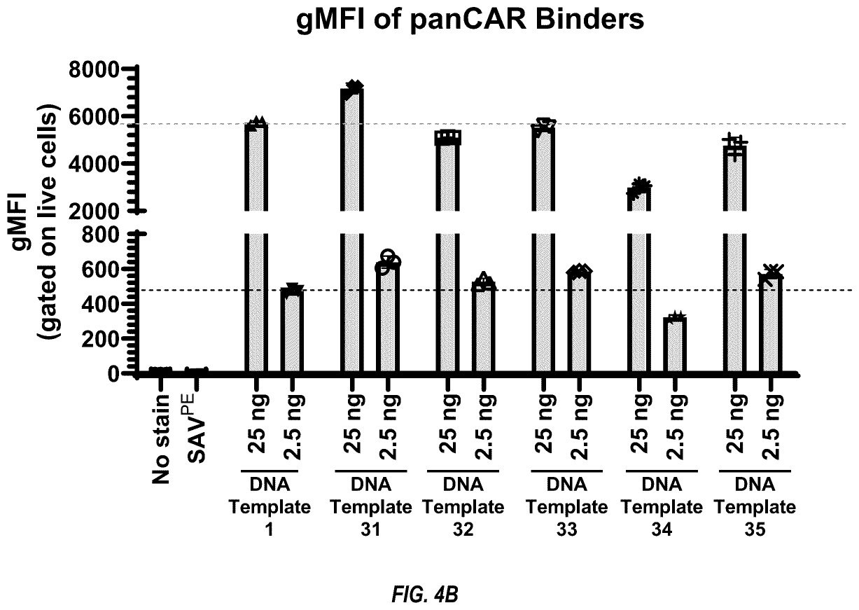

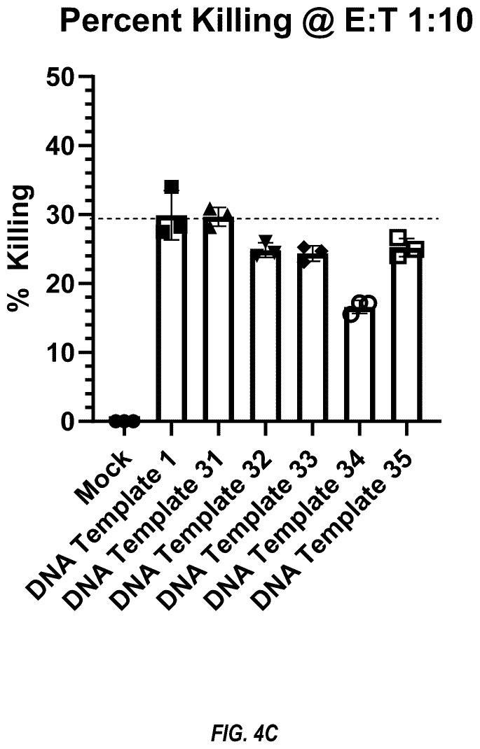

6 circular RNAs encoding anti-CD19 chimeric antigen receptor (CAR) candidates were tested to express in vitro. Human T cells were activated for 3 days with anti-CD3 and anti-CD28 solution and allowed to rest for 24 hours. Concurrently, engineered circular RNA constructs were designed to encode a CD19-CD28ζ chimeric antigen receptor (CAR). For comparison purposes, “mock” T cells were not electroporated with circular RNA. The circular RNAs were developed from an in vitro translation (IVT) reaction of DNA (e.g., DNA Templates 1, 31-25 of Table 00) comprising a T7 polymerase promoter, permuted Anabaena intron exon segments, internal ribosome entry site (IRES), internal spacers, optionally internal homology arms, and a Xlab restriction site. The circular RNAs were engineered to comprise an internal ribosome entry site (IRES) derived from and/or belonging to Hunnivirus. Anti-CD19 CAR sequences used comprised of Binders A-E or X as shown in Table 00. DNA templates comprised of sequences from the table below. Human T cells were electroporated with the oRNA at 25 ng or 5 ng per 0.1×10⁶ T cells to form CAR-T cells respectively (FIGS. 4A-4C). As a control, “Mock” T cells not electroporated with circular RNAs were analyzed. T cells were then allowed to rest for 24 hours after transfection. 24 hours after transfection, the CAR-T cells were counted and assessed for anti-CD19 CAR expression. Resulting oCAR-T cells were given a commercially available biotinylated soluble human CD19 detection reagent (e.g., from Miltenyi Biotec, Bergisch Gladbach) and Streptavidin conjugated to R-phycoerythrin (PE) fluorophores (e.g., from Biolegend, San Diego), or commercially available anti-cynomolgus CD19 detection reagent (e.g., from KactusBio, Waltham Massachusetts). Anti-CD19 expression was assessed using fluorescence activated cell sorting (FACS) and gating flow cytometry methods known in the art at one or more timepoints from 24 hours post transfection. CAR expression and intensity as depicted from the soluble CD19 detection reagent is shown in FIGS. 4A-4B for the human T cells tested.

Selected oCAR-T cells were co-cultured with targeted Nalm6 cells E:T ratios (i.e., 1:10 ratio) 24-hours following electroporation of the oRNA to the donor T cells. Cytotoxicity of CD19 targeted killing on Nalm6 cells was studied via a commercially available firefly luciferase (FLuc) assay. 48 hours post electroporation of the circular RNA into the T cells, oCAR-T cells were sub-gated on live T cells based on the FLuc assay results and amount of detectable reagent was collected. The oCAR-T cells were analyzed using a commercially available firefly luciferase assay and analyzed 48 hours post co-culture for cytotoxicity. Resulting human CD3+ T cells co-cultured with Nalm6 cytotoxicity levels are shown in FIG. 4C.

TABLE ββ

| DNA | Construct Number | | Costim | |
|---|---|---|---|---|
| Template # | from Table 5 | Binder (s) | domain | IRES |
| 1 | Construct Q | Binder X | CD28ζ | IRES A |
| 32 | Construct AV | Binder B (Table | CD28ζ | IRES A |
| | | 10, #20) | | |
| 33 | Construct AW | Binder C (Table | CD28ζ | IRES A |
| | | 10, #21) | | |
| 34 | Construct AX | Binder D (Table | CD28ζ | IRES A |
| | | 10, #22) | | |
| 35 | Construct AY | Binder E (Table | CD28ζ | IRES A |
| | | 10, #23) | | |

### Example 6: oCAR Frequency and Intensity of Anti-CD19 CARs Expression in Human and Cynomolgus T Cells

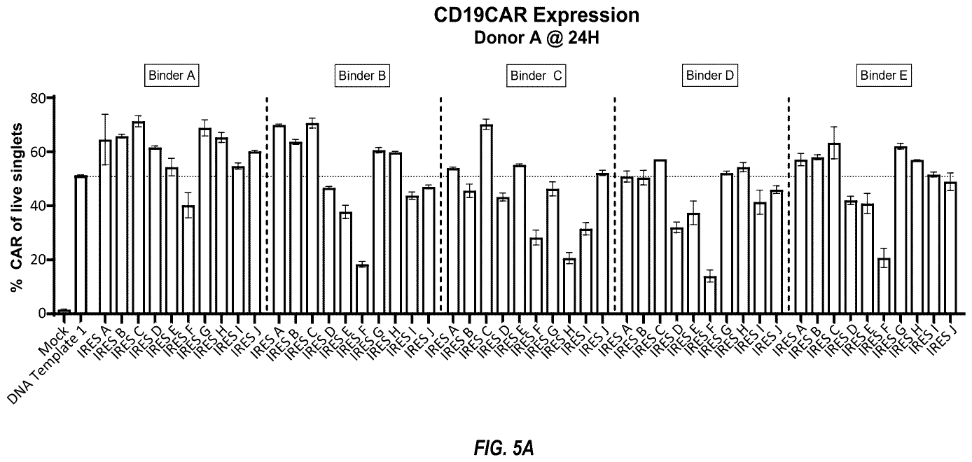

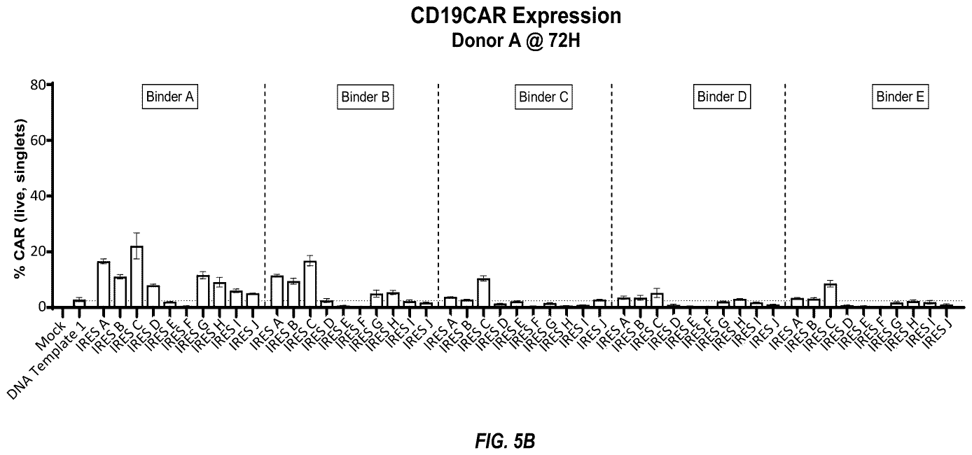

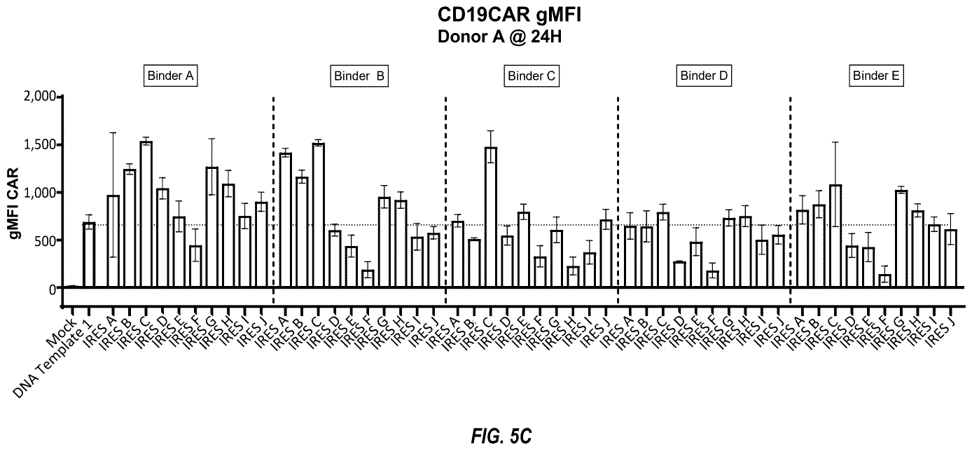

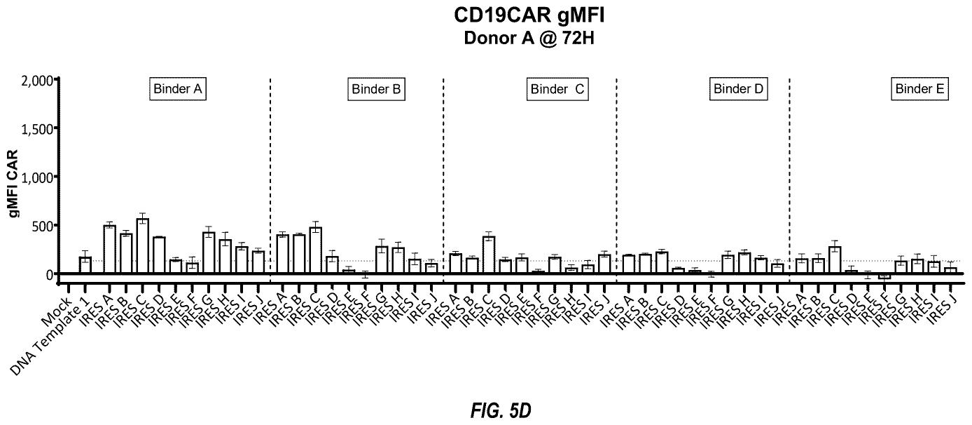

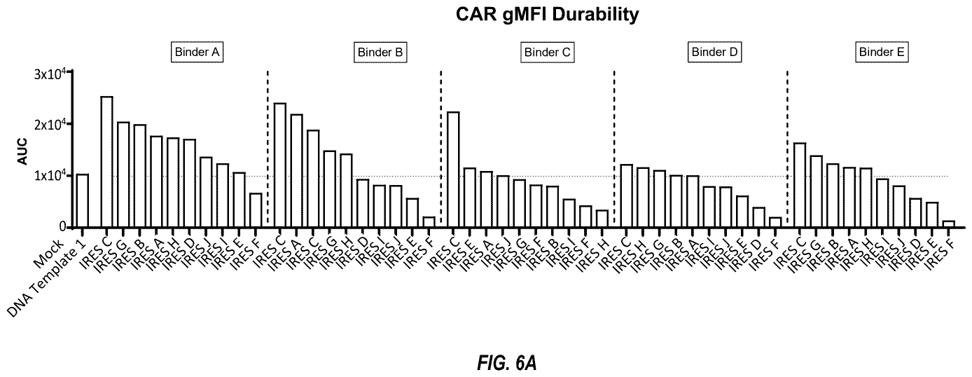

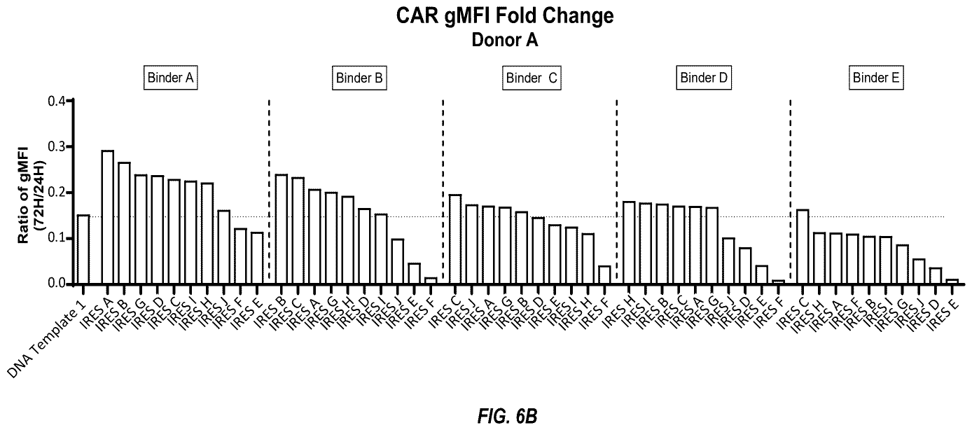

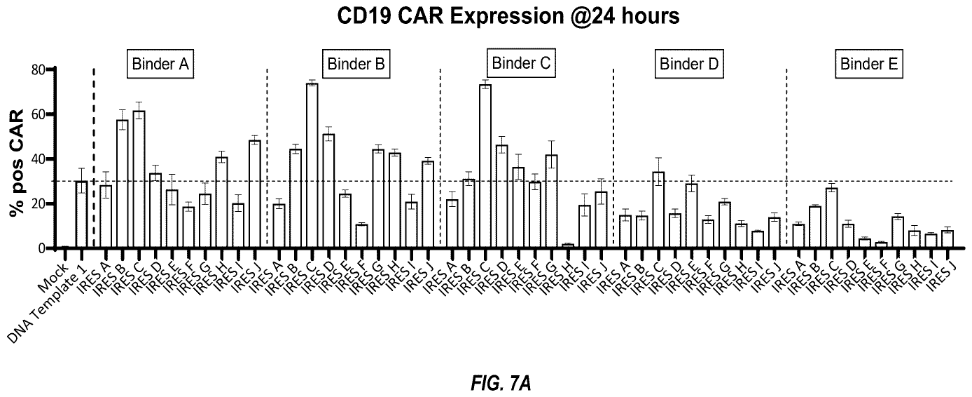

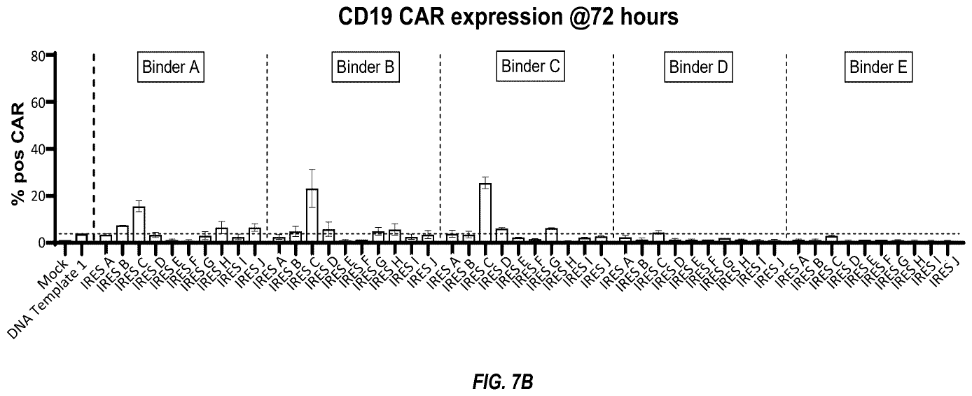

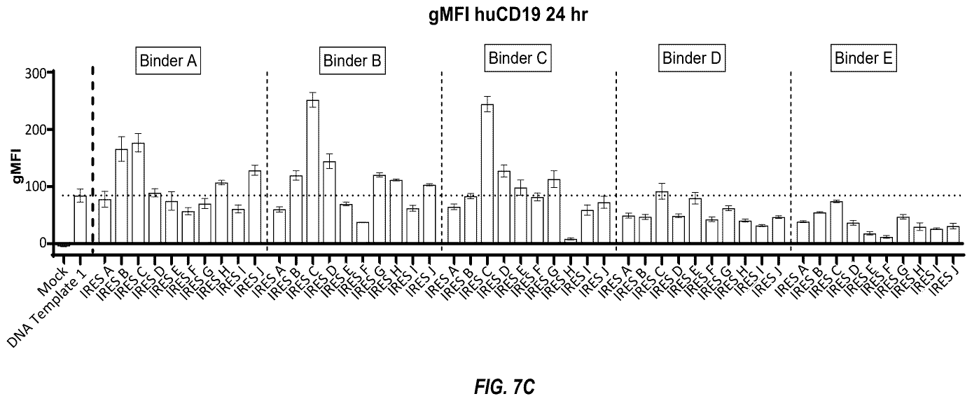

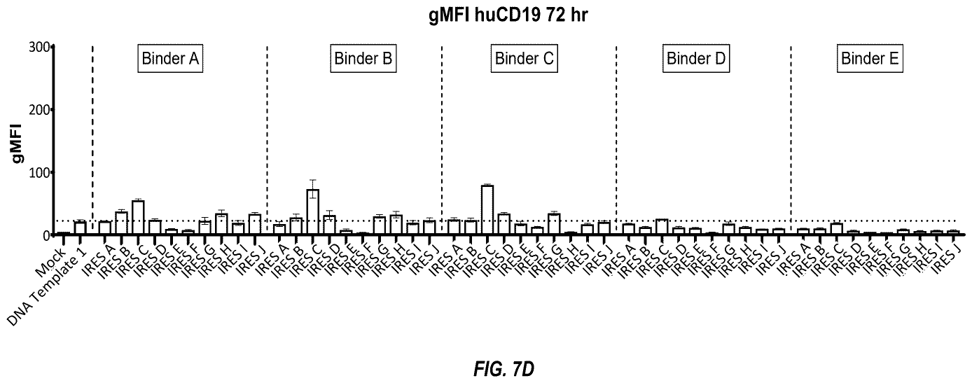

51 circular RNAs encoding anti-CD19 chimeric antigen receptor (CAR) candidates were tested to express in vitro. Human and cynomolgus T cells were activated for 3 days with anti-CD3 and anti-CD28 solution and allowed to rest for 24 hours. Concurrently, engineered circular RNA constructs were designed to encode a CD19-CD28ζ chimeric antigen receptor (CAR). For comparison purposes, “mock” T cells were not transfected. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs were engineered to comprise an internal ribosome entry site (IRES). Anti-CD19 CAR sequences used comprised of Binders A-E or X as shown in Table 7T. DNA templates comprised of sequences from the table below. Circular RNAs were formulated with LNPs to form oRNA-LNPs (Formulation A). Human and cynomolgus donor T cells were transfected with the oRNA-LNPs at 100 ng or 200 ng per 0.1×10⁶ T cells to form CAR-T cells respectively (FIGS. 5A-5D, FIGS. 6A-6B, and FIGS. 7A-7D). As a control, “Mock” T cells not transfected with circular RNAs were analyzed. T cells were then allowed to rest for 24 hours after transfection. 24 and 72 hours after transfection, the CAR-T cells were counted and assessed for CD19 CAR expression. Resulting oCAR-T cells were given a commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 expression was assessed using fluorescence activated cell sorting (FACS) and gating flow cytometry methods known in the art at one or more timepoints from 24 hours post transfection. Durability of CAR expression was measured by calculating total expression over 72 hours post transfection (e.g., via area under the curve (AUC) of anti-CD19 CAR expression graph) (illustrated in FIG. 6A). Decay of CAR expression at 72 hours was calculated by determining fold change of geometric mean fluorescent intensity (gMFI) between 72 and 24 hours post transfection (i.e., [gMFI at 72 hours]/[gMFI at 24 hours]) (illustrated in FIG. 6B) CAR expression and intensity as depicted from the soluble CD19 detection reagent is shown in FIGS. 7A-7D for the human T cells tested and FIGS. 5A-5D for the cynomolgus T cells tested.

TABLE γγ

| DNA | Construct Number | | Costim | |
|---|---|---|---|---|
| Template # | from Table 5 | Binder (s) | domain | IRES |
| 1 | Construct Q | Binder X | CD28ζ | IRES A |
| 32 | Construct AV | Binder B | CD28ζ | IRES A |
| 45 | Construct BI | Binder B | CD28ζ | IRES B |
| 46 | Construct BJ | Binder B | CD28ζ | IRES C |
| 47 | Construct BK | Binder B | CD28ζ | IRES D |
| 48 | Construct BL | Binder B | CD28ζ | IRES E |
| 49 | Construct BM | Binder B | CD28ζ | IRES F |
| 50 | Construct BN | Binder B | CD28ζ | IRES G |
| 51 | Construct BO | Binder B | CD28ζ | IRES H |
| 52 | Construct BP | Binder B | CD28ζ | IRES I |
| 53 | Construct BQ | Binder B | CD28ζ | IRES J |
| 33 | Construct AW | Binder C | CD28ζ | IRES A |
| 54 | Construct BR | Binder C | CD28ζ | IRES B |
| 55 | Construct BS | Binder C | CD28ζ | IRES C |
| 56 | Construct BT | Binder C | CD28ζ | IRES D |
| 57 | Construct BU | Binder C | CD28ζ | IRES E |
| 58 | Construct BV | Binder C | CD28ζ | IRES F |
| 59 | Construct BW | Binder C | CD28ζ | IRES G |
| 60 | Construct BX | Binder C | CD28ζ | IRES H |
| 61 | Construct BY | Binder C | CD28ζ | IRES I |
| 62 | Construct BZ | Binder C | CD28ζ | IRES J |
| 34 | Construct AX | Binder D | CD28ζ | IRES A |
| 63 | Construct CA | Binder D | CD28ζ | IRES B |
| 64 | Construct CB | Binder D | CD28ζ | IRES C |
| 65 | Construct CC | Binder D | CD28ζ | IRES D |
| 66 | Construct CD | Binder D | CD28ζ | IRES E |
| 67 | Construct CE | Binder D | CD28ζ | IRES F |
| 68 | Construct CF | Binder D | CD28ζ | IRES G |
| 69 | Construct CG | Binder D | CD28ζ | IRES H |
| 70 | Construct CH | Binder D | CD28ζ | IRES I |
| 71 | Construct CI | Binder D | CD28ζ | IRES J |
| 35 | Construct AY | Binder E | CD28ζ | IRES A |
| 72 | Construct CJ | Binder E | CD28ζ | IRES B |
| 73 | Construct CK | Binder E | CD28ζ | IRES C |
| 74 | Construct CL | Binder E | CD28ζ | IRES D |
| 75 | Construct CM | Binder E | CD28ζ | IRES E |
| 76 | Construct CN | Binder E | CD28ζ | IRES F |
| 77 | Construct CO | Binder E | CD28ζ | IRES G |
| 78 | Construct CP | Binder E | CD28ζ | IRES H |
| 79 | Construct CQ | Binder E | CD28ζ | IRES I |
| 80 | Construct CR | Binder E | CD28ζ | IRES J |

### Example 7: oCAR Expression and Percent Killing of Anti-CD19 CARs in Human T Cells

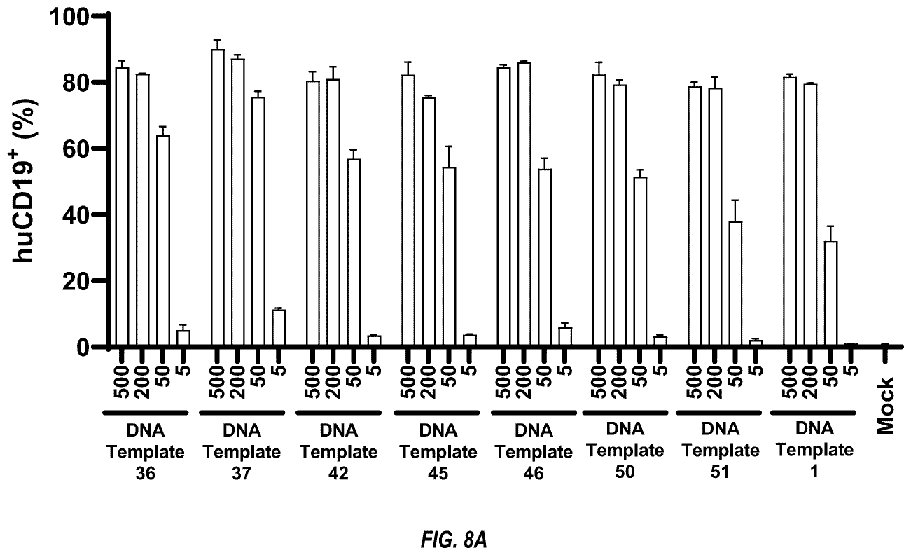

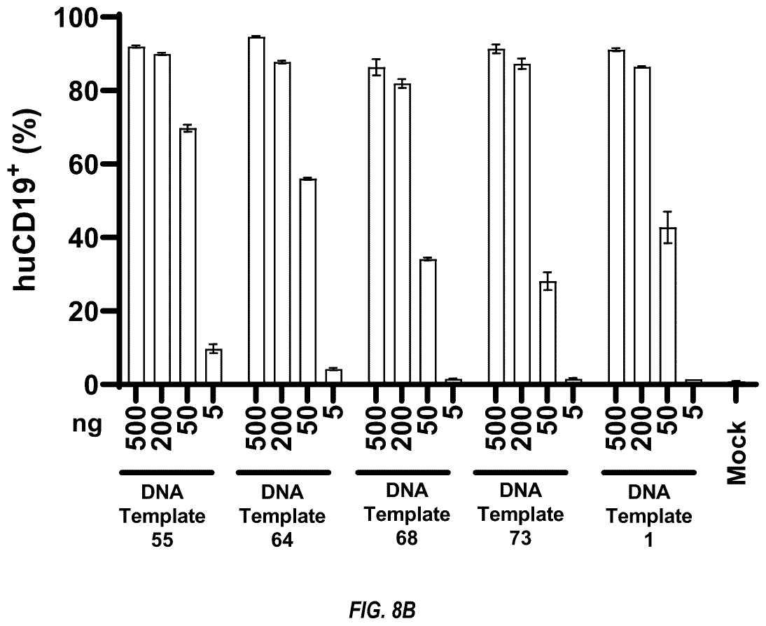

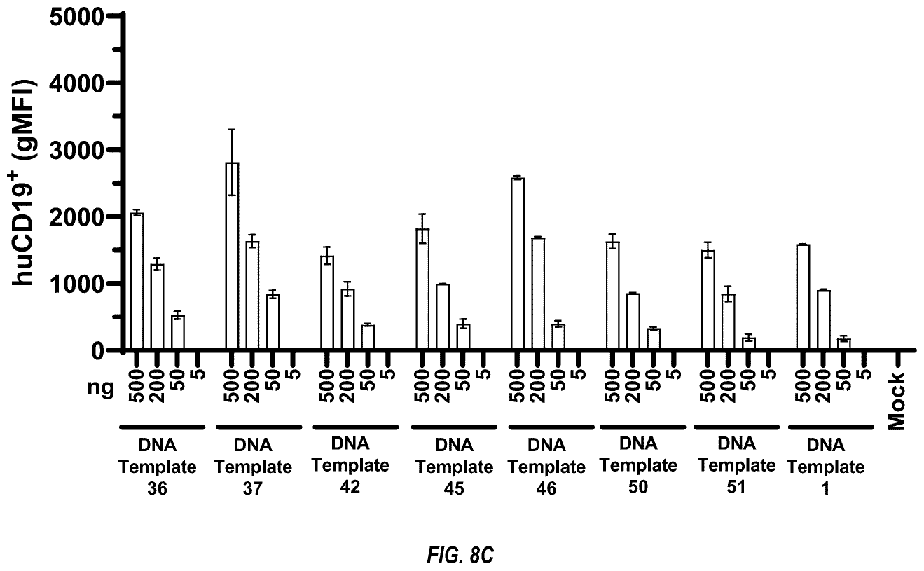

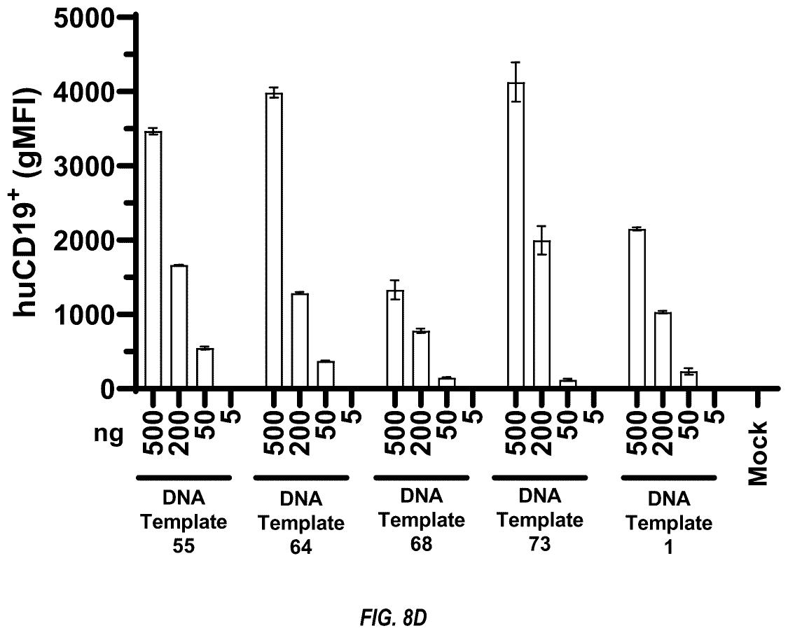

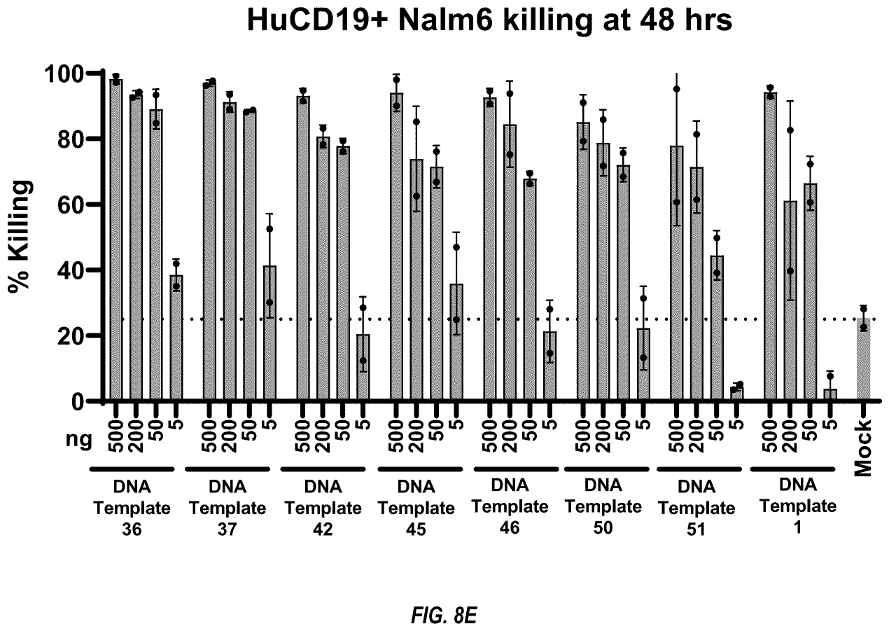

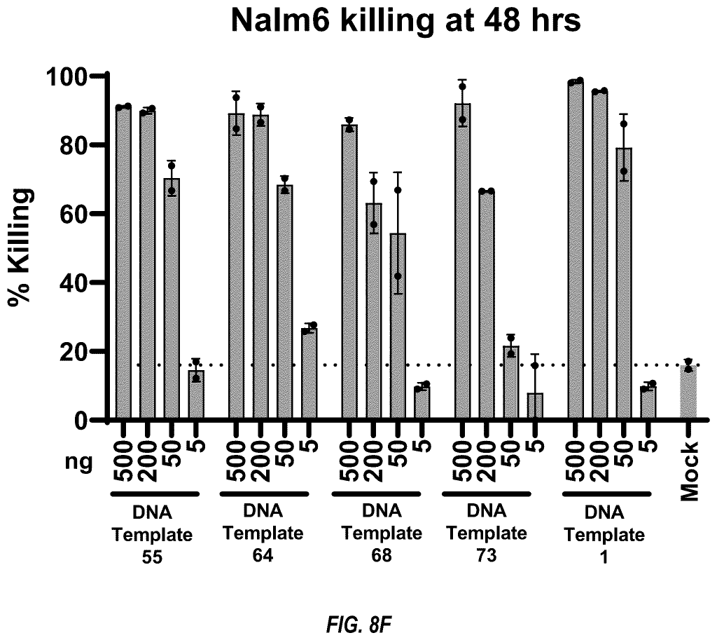

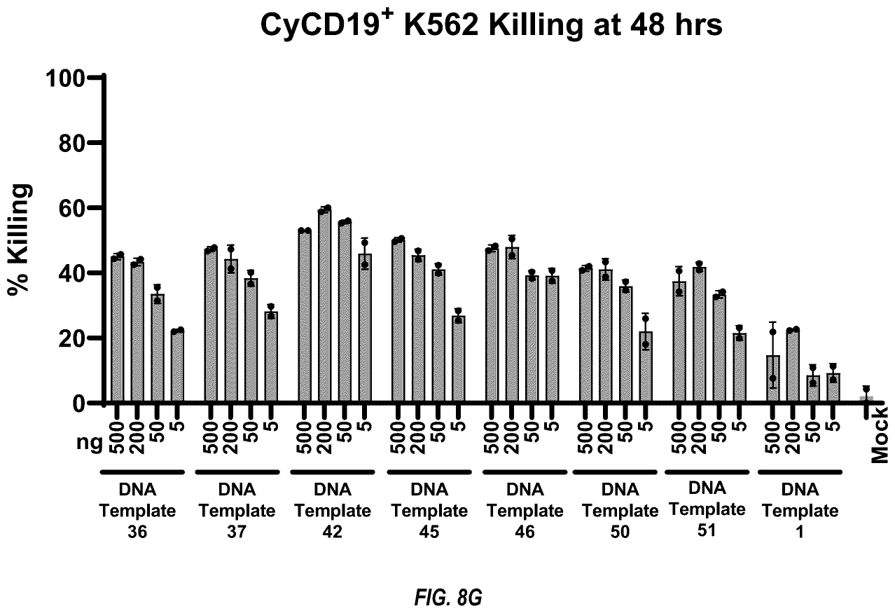

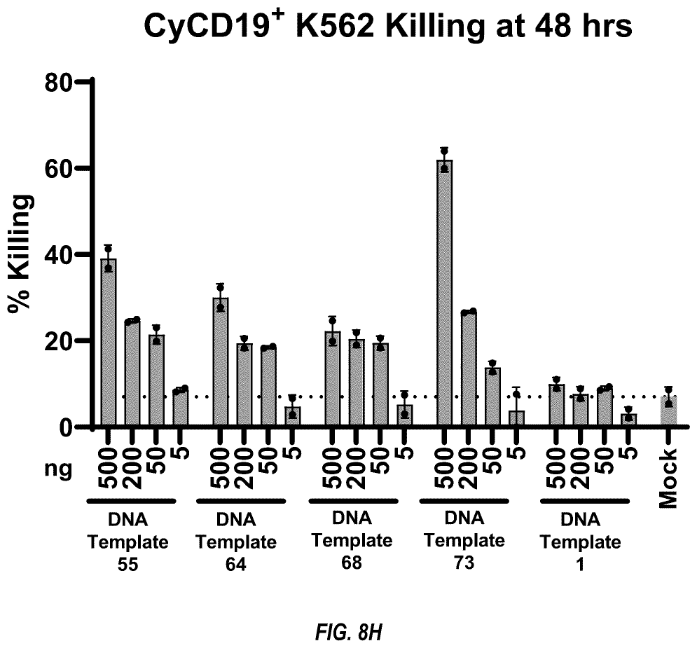

12 circular RNAs encoding anti-CD19 chimeric antigen receptor (CAR) candidates were tested to express in vitro. Human T cells were activated for 3 days with anti-CD3 and anti-CD28 solution and allowed to rest for 24 hours. Concurrently, engineered circular RNA constructs were designed to encode a CD19-CD28ζ chimeric antigen receptor (CAR). For comparison purposes, “mock” T cells were not transfected. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs were engineered to comprise an internal ribosome entry site (IRES). Anti-CD19 CAR sequences used comprised of Binder X, A-E as shown in Table γγ. DNA templates comprised of DNA Template 1 from Table ββ and DNA Templates 36-37, 42, 46-47, 51-52, 57, 67, 71, 77s from the Table γγ. Circular RNAs were formulated with LNPs to form oRNA-LNPs (Formulation A). Human donor T cells were transfected with the oRNA-LNPs at 500 ng, 200 ng, 50 ng, or 5 ng per 0.1×10 T cell to form CAR-T cells (FIGS. 8A-8D and 8E-8H). As a control, “Mock” T cells not transfected with oRNA-LNPs were analyzed. T cells were then allowed to rest for 24 hours after transfection. 24 hours after transfection, the CAR-T cells were counted and assessed for CD19 CAR expression. Resulting oCAR-T cells were given a commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 expression was assessed using fluorescence (e.g., via a commercially available firefly luciferase assay) and gating flow cytometry methods known in the art at one or more timepoints from 24 hours post transfection. CAR expression and intensity as depicted from the soluble CD19 detection reagent is shown in FIGS. 8A-8D for the human T cells tested.

Selected oCAR-T cells were co-cultured with targeted Nalm6 or humanized cynomolgus CD19 (K562.cyCD19) cells at an E:T ratio (i.e., 1:10 ratio) 24-hours following transfection of the oRNA-LNPs to the donor T cells. 48 hours later, cytotoxicity of CD19 targeted killing on Nalm6 or K562.cyCD19 was studied via FACS. Resulting human CD3+ T cells co-cultured with Nalm6 or K562.cyCD19 cytotoxicity levels are shown in FIG. 8E-8H.

### Example 8: oCAR Expression, Durability and Decay of Anti-CD19 CARs in Human and Cynomolgus T Cells

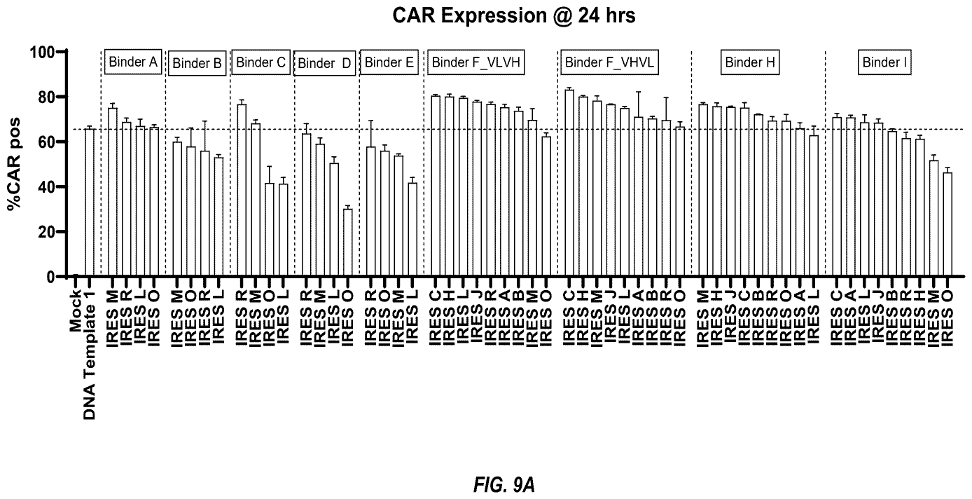

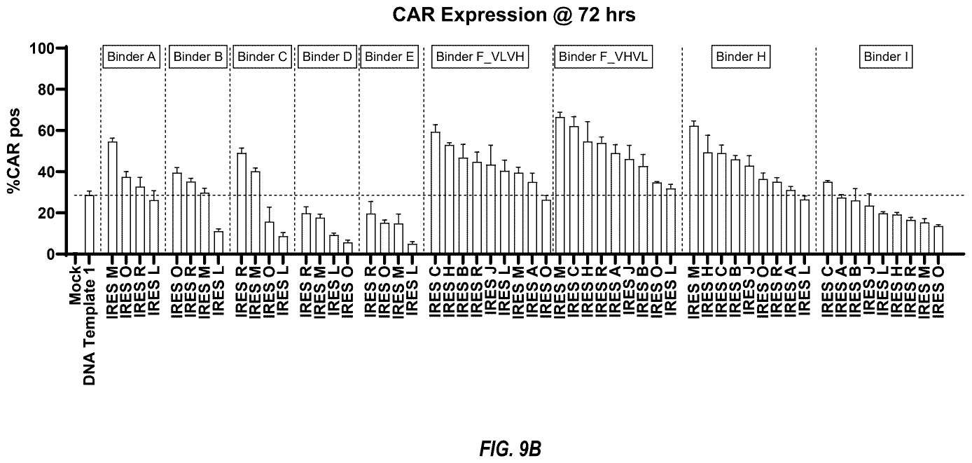

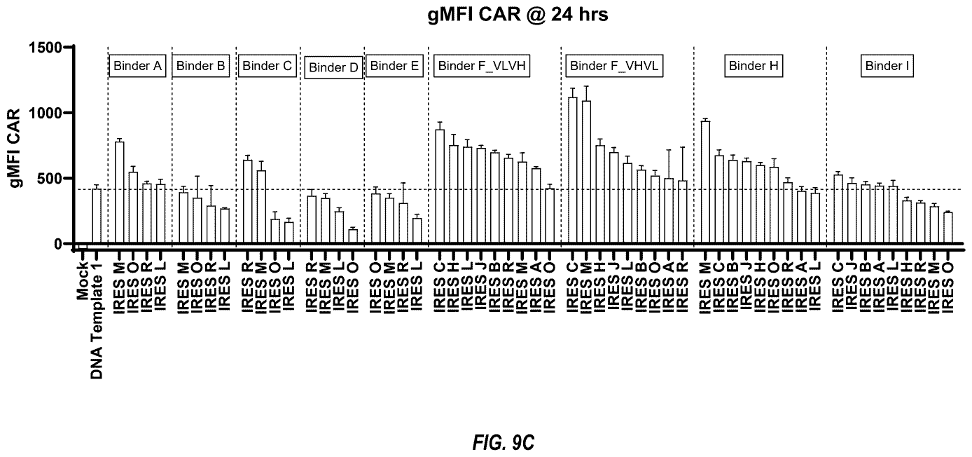

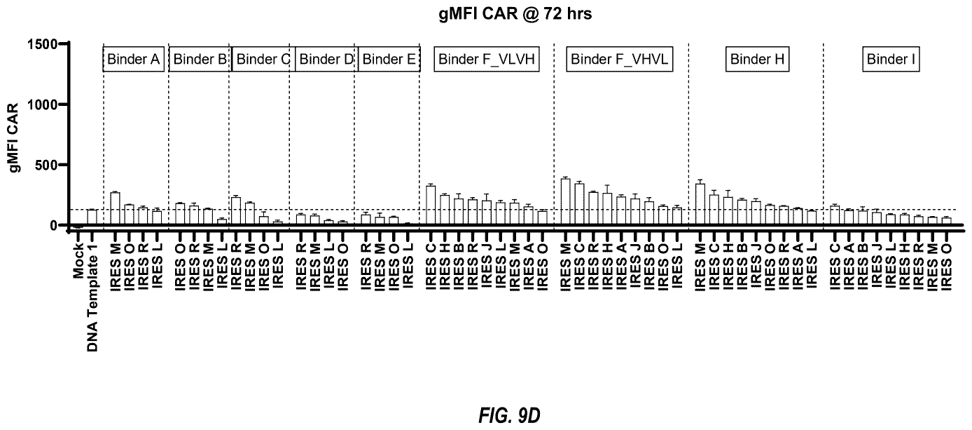

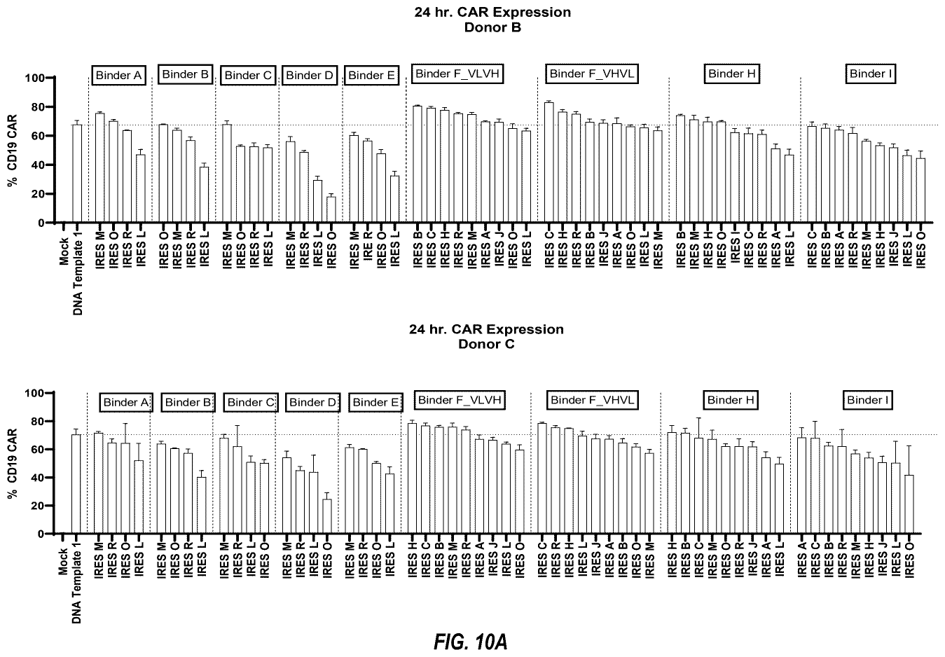

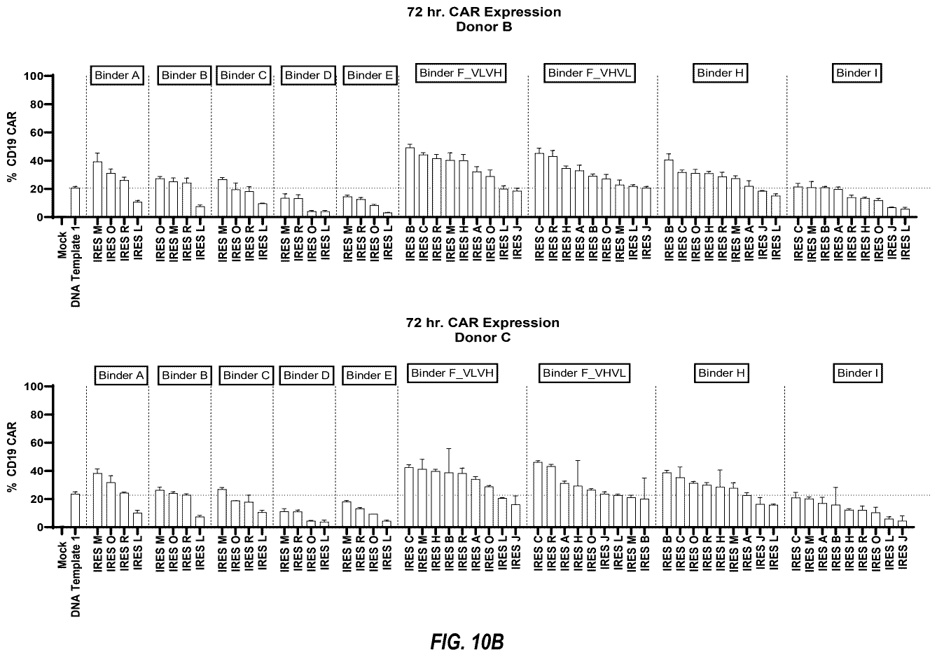

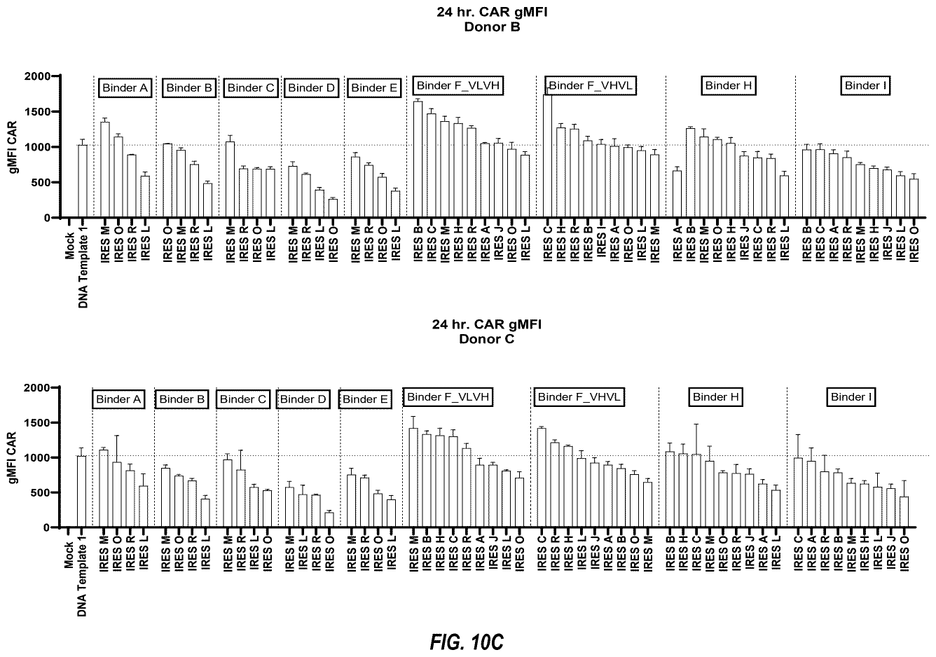

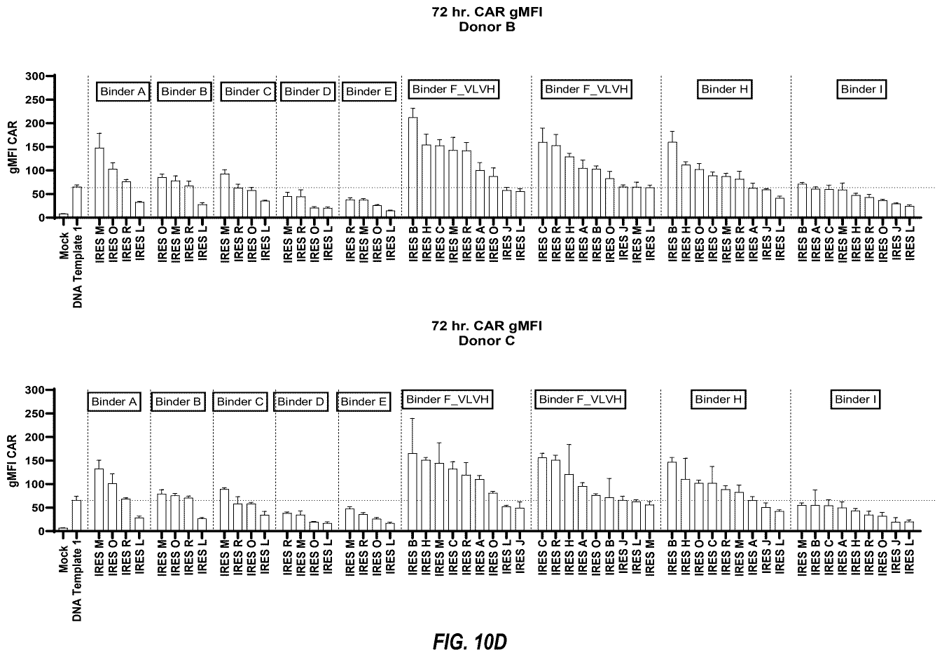

57 circular RNAs encoding anti-CD19 chimeric antigen receptor (CAR) candidates were tested to express in vitro. Human and cynomolgus T cells were activated for 3 days with anti-CD3 and CD28 solution and allowed to rest for 24 hours. Concurrently, engineered circular RNA constructs were designed to encode a CD19-CD28ζ chimeric antigen receptor (CAR). For comparison purposes, “mock” T cells were not transfected. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs were engineered to comprise an internal ribosome entry site (IRES). Anti-CD19 CAR sequences used comprised of Binder B-I as shown in Table 66. DNA templates comprised of sequences from Table 66. Circular RNAs were formulated with LNPs to form oRNA-LNPs (Formulation A). Human and cynomolgus donor T cells were transfected with the oRNA-LNPs at 100 ng or 200 ng per 0.1×10⁶ T cells to form CAR-T cells respectively (FIGS. 9A-9D and FIGS. 10A-10D). As a control, “Mock” T cells not transfected with circular RNAs were analyzed. T cells were then allowed to rest for 24 hours after transfection. 24 and 72 hours after transfection, the CAR-T cells were counted and assessed for CD19 CAR expression. Resulting oCAR-T cells were given a commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 expression was assessed using fluorescence activated cell sorting (FACS) and gating flow cytometry methods known in the art at one or more timepoints from 24 hours post transfection. CAR expression and intensity as depicted from the soluble CD19 detection reagent is shown in FIGS. 9A-9D for the human T cells tested and FIGS. 10A-10D for the cynomolgus T cells tested.

TABLE δδ

| DNA | Construct Number | | Costim | |
|---|---|---|---|---|
| Template # | from Table 5 | Binder (s) | domain | IRES |
| 1 | Construct Q | Binder X | CD28ζ | IRES A |
| 81 | Construct CS | Binder B | CD28ζ | IRES O |
| 82 | Construct CT | Binder C | CD28ζ | IRES O |
| 83 | Construct CU | Binder B | CD282 | IRES R |
| 84 | Construct CV | Binder F (Table | CD28ζ | IRES R |
| | | 10, #24) | | |
| 85 | Construct CW | Binder E | CD28ζ | IRES R |
| 86 | Construct CX | Binder F | CD28ζ | IRES A |
| 87 | Construct CY | Binder G (Table | CD28ζ | IRES L |
| | | 10, #25) | | |
| 88 | Construct CZ | Binder G | CD28ζ | IRES C |
| 90 | Construct DB | Binder G | CD28ζ | IRES R |
| 91 | Construct DC | Binder D | CD28ζ | IRES R |
| 92 | Construct DD | Binder F | CD28ζ | IRES C |
| 93 | Construct DE | Binder H (Table | CD28ζ | IRES O |
| | | 10, #26) | | |
| 94 | Construct DF | Binder F | CD28ζ | IRES M |
| 95 | Construct DG | Binder H | CD28ζ | IRES A |
| 96 | Construct DH | Binder H | CD28ζ | IRES R |
| 97 | Construct DI | Binder B | CD28ζ | IRES L |
| 98 | Construct DJ | Binder H | CD28ζ | IRES L |
| 99 | Construct DK | Binder D | CD28ζ | IRES M |
| 100 | Construct DL | Binder G | CD28ζ | IRES O |
| 101 | Construct DM | Binder F | CD28ζ | IRES O |
| 102 | Construct DN | Binder I (Table | CD28ζ | IRES M |
| | | 10, #27) | | |
| 104 | Construct DP | Binder G | CD28ζ | IRES A |
| 105 | Construct DQ | Binder G | CD28ζ | IRES M |
| 106 | Construct DR | Binder | CD28ζ | IRES C |
| 108 | Construct DT | Binder I | CD28ζ | IRES O |
| 109 | Construct DU | Binder I | CD28ζ | IRES R |
| 110 | Construct DV | Binder I | CD28ζ | IRES C |
| 111 | Construct DW | Binder H | CD28ζ | IRES M |
| 112 | Construct DX | Binder I | CD28ζ | IRES L |
| 113 | Construct DY | Binder I | CD28ζ | IRES A |
| 114 | Construct DZ | Binder D | CD28ζ | IRES L |
| 115 | Construct EA | Binder C | CD28ζ | IRES R |
| 117 | Construct EC | Binder E | CD28ζ | IRES M |
| 118 | Construct ED | Binder C | CD28ζ | IRES L |
| 119 | Construct EE | Binder E | CD28ζ | IRES O |
| 120 | Construct EF | Binder D | CD28ζ | IRES O |
| 121 | Construct EG | Binder C | CD28ζ | IRES M |
| 122 | Construct EH | Binder B | CD28ζ | IRES M |
| 123 | Construct EI | Binder E | CD28ζ | IRES L |
| 124 | Construct EJ | Binder F | CD28ζ | IRES L |
| 125 | Construct EK | Binder I | CD28ζ | IRES H |
| 126 | Construct EL | Binder G | CD28ζ | IRES H |
| 127 | Construct EM | Binder H | CD28ζ | IRES B |
| 128 | Construct EN | Binder G | CD28ζ | IRES B |
| 129 | Construct EO | Binder F | CD28ζ | IRES H |
| 130 | Construct EP | Binder I | CD28ζ | IRES B |
| 131 | Construct EQ | Binder F | CD28ζ | IRES B |
| 132 | Construct ER | Binder I | CD28ζ | IRES J |
| 133 | Construct ES | Binder H | CD28ζ | IRES H |
| 134 | Construct ET | Binder G | CD28ζ | IRES J |
| 135 | Construct EU | Binder H | CD28ζ | IRES J |
| 136 | Construct EV | Binder F | CD28ζ | IRES J |

### Example 9: oCAR Expression of Codon Optimized or Other Altered Anti-CD19 CARs in Cynomolgus and Human T Cells

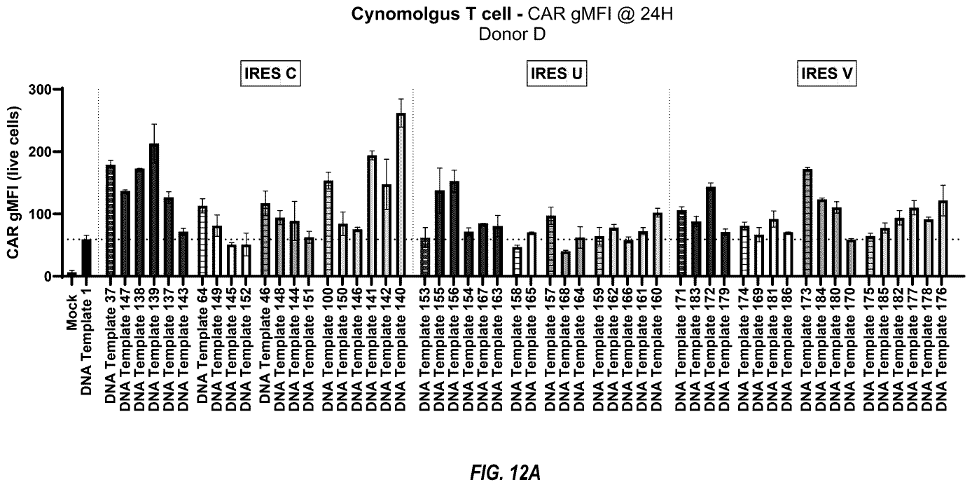

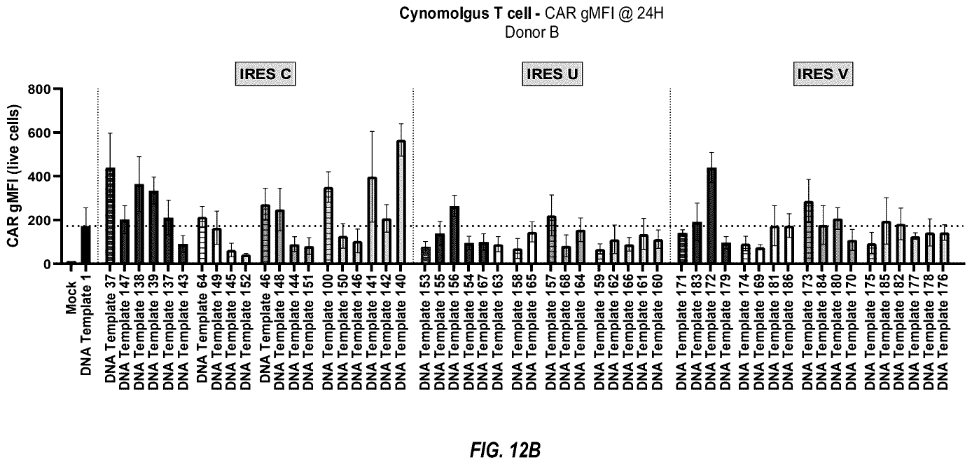

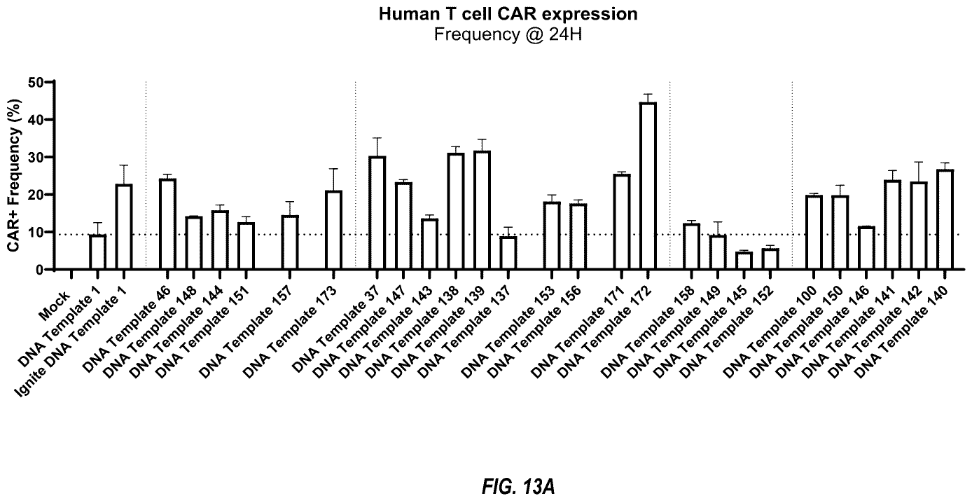

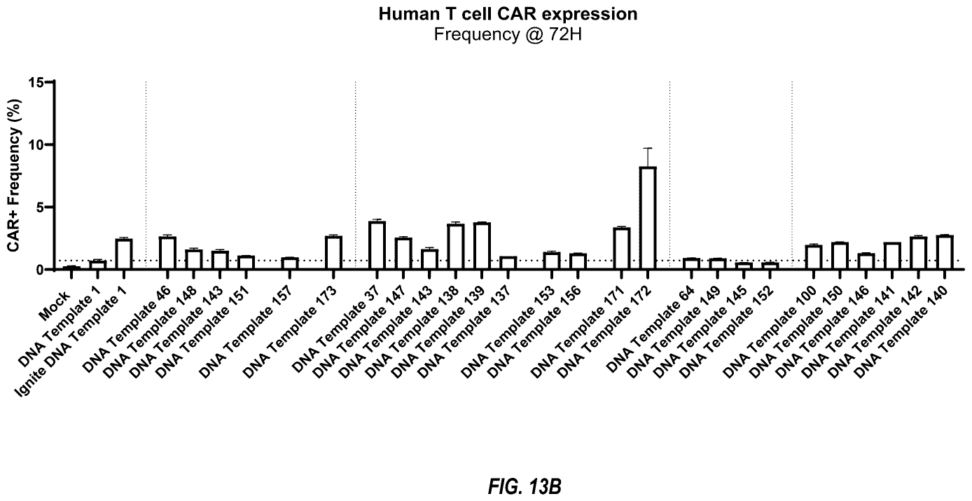

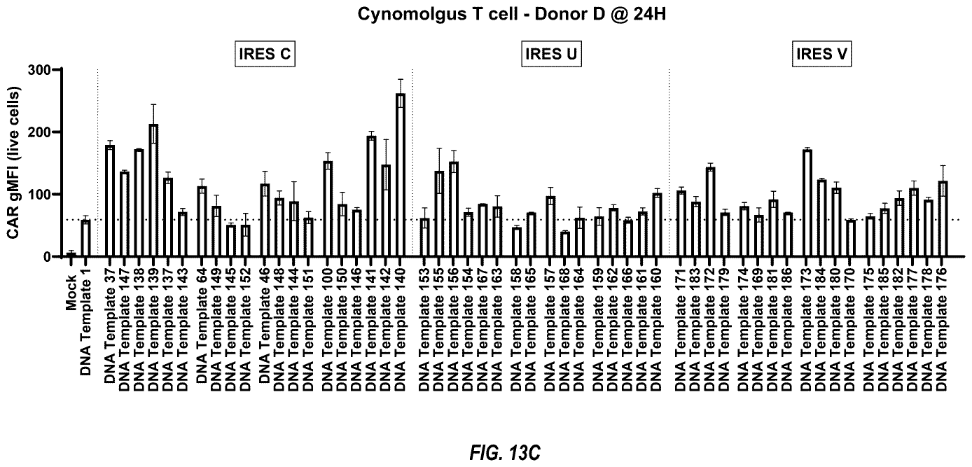

55 circular RNAs encoding anti-CD19 chimeric antigen receptor (CAR) candidates were tested to express in vitro. Cynomolgus T cells from two donors (e.g., Donor B and Donor D) were activated for 3 days with anti-CD2, anti-CD3 and anti-CD28 beads, and allowed to rest for 24 hours. Concurrently, engineered circular RNA constructs were designed to encode a CD19-CD28 CAR. For comparison purposes, “mock” or control circular RNAs encoded a HER2. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs comprise various internal ribosome entry sites (IRES). CAR sequences tested comprise of Binders A, B, D, I-Z, or AA as shown in Table ωω. DNA templates (e.g., DNA Templates from Table ωω) comprising alterations including codon optimizations, lysine to arginine mutations, and linker extensions (e.g., additions of one or more G4S linker sequences (SEQ ID NO: 1)). Circular RNAs were formulated to form oRNA-LNPs (Formulation A). Cynomolgus donor T cells were transfected with the circular RNAs at 200 ng per 0.1×10⁶ T cell to form CAR-T cells. (FIG. 12A-12B and FIG. 13C). As a control, “Mock” T cells not transfected with circular RNAs were analyzed. 24 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at 24 hours post transfection. CAR expression intensity as detected from the soluble CD19 detection reagent is shown in FIGS. 12A-12B and FIG. 13C for the cynomolgus T cells tested.

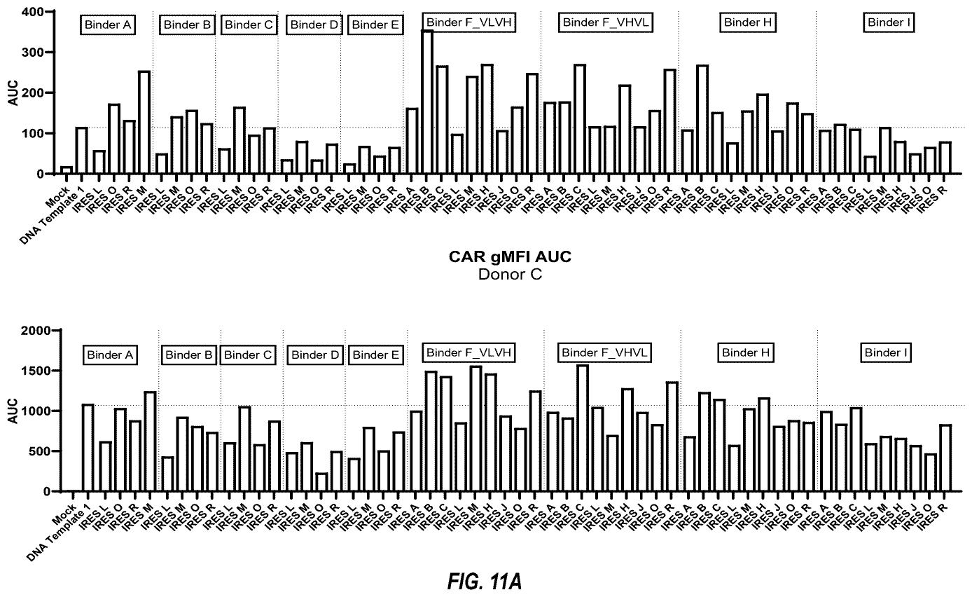

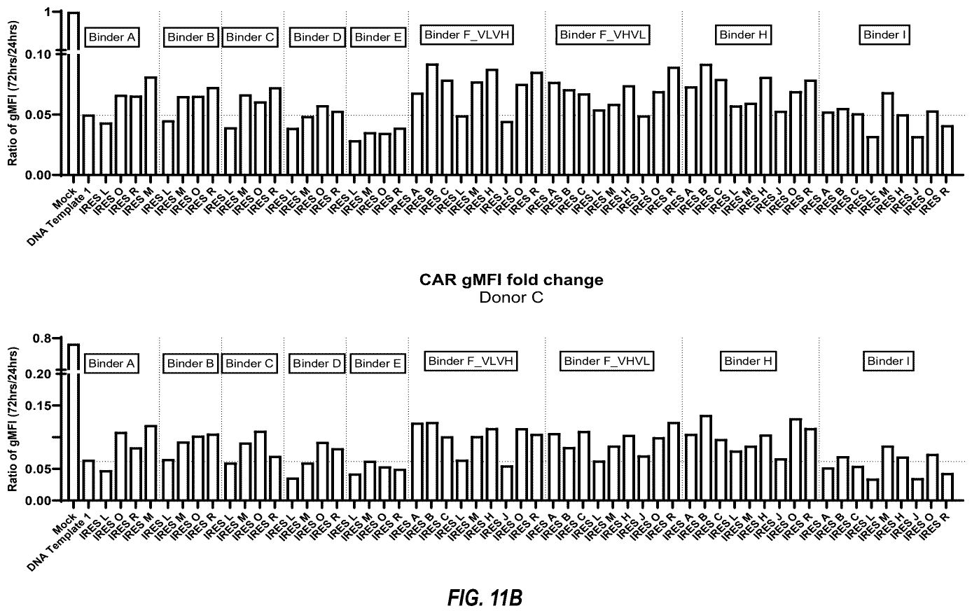

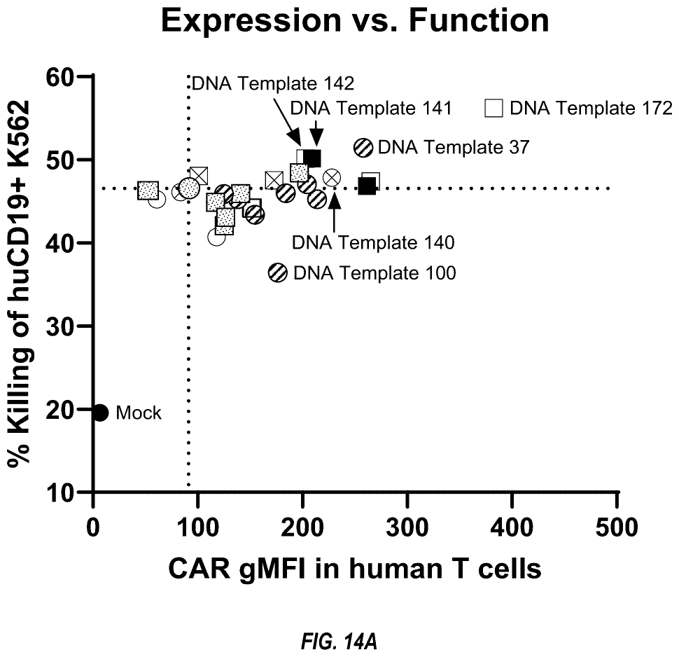

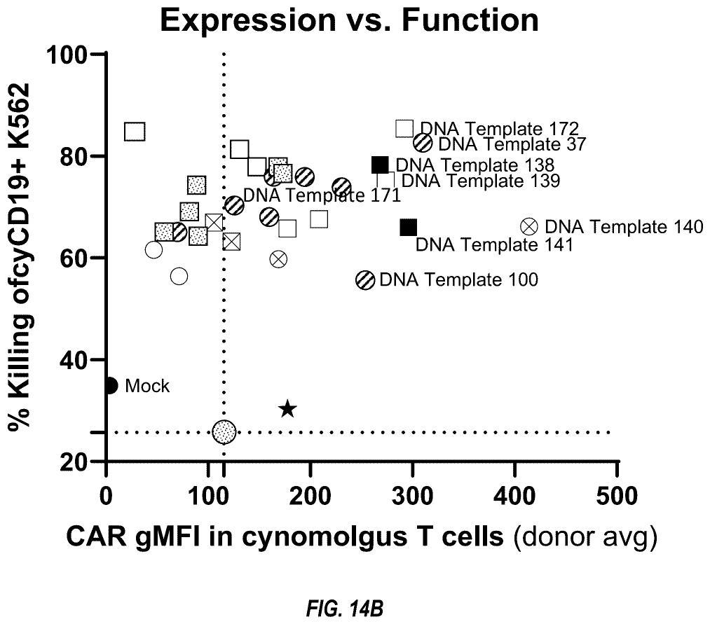

27 circular RNAs encoding anti-CD19 chimeric antigen receptor (CAR) candidates selected from the 54 circular RNAs were tested to express in vitro. Human T cells from one donor (e.g., Donor were cultured for one day, activated with a soluble anti-CD3 and CD28 cocktail for 3 days, and allowed to rest for 24 hours. Human donor T cells were transfected with the circular RNAs at 84 ng per 0.1×10⁶ T cell to form CAR-T cells. (FIGS. 13A-13B, 14A-14B). As a control, “Mock” T cells not transfected with circular RNAs were analyzed. 24 and 72 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at one or more timepoints from 24 hours post transfection. CAR expression frequency as detected from the soluble CD19 detection reagent is shown in FIGS. 13A-13B for the human T cells tested. Durability of CAR expression was measured by calculating total expression over 72 hours post transfection (e.g., via area under the curve (AUC) of anti-CD19 CAR expression graph) (illustrated in FIG. 11A). Decay of CAR expression at 72 hours was calculated by determining foldchange of geometric mean fluorescent intensity (gMFI) between 72 and 24 hours post transfection (i.e., [IgMFI at 72 hours]/[IgMFI at 24 hours]) (illustrated in FIG. 11B).

24 hours post transfection, the oCAR-T cells generated from the 27 circular oRNAs encoding anti-CD19 CAR sequences were co-cultured with a tumor cell line (e.g. K562 cell line) and engineered to overexpress either soluble human CD19 (huCD19) protein or soluble cynomolgus CD19 (cyCD19) protein at a 1:0.5 or 1:1 E:T ratio, respectively. Cytotoxicity of CD19 targeted killing against huCD9+K562 and cyCD9+K562 was studied via FACS after 48 hours of continuous co-culture. Resulting cytotoxicity levels are shown in FIGS. 14A-14B.

TABLE ωω

| DNA | | | | |
|---|---|---|---|---|
| Template | Construct Number | | Costim | |
| # | from Table 5 | Binder (s) | domain | IRES |
| 1 | Construct Q | Binder X | CD28ζ | IRES A |
| 46 | Construct BJ | Binder B | CD28ζ | IRES C |
| 64 | Construct CB | Binder D | CD28ζ | IRES C |
| 100 | Construct DV | Binder I | CD28ζ | IRES C |
| 140 | Construct EZ | Binder M (Table | CD28ζ | IRES C |
| | | 10, #31) | | |
| 141 | Construct FA | Binder N (Table | CD28ζ | IRES C |
| | | 10, #32) | | |
| 142 | Construct FB | Binder O (Table | CD28ζ | IRES C |
| | | 10, #33) | | |
| 144 | Construct FD | Binder Q (Table | CD28ζ | IRES C |
| | | 10, #35) | | |
| 145 | Construct FE | Binder R (Table | CD28ζ | IRES C |
| | | 10, #36) | | |
| 146 | Construct FF | Binder S (Table | CD28ζ | IRES C |
| | | 10, #37) | | |
| 148 | Construct FH | Binder U (Table | CD28ζ | IRES C |
| | | 10, #39) | | |
| 149 | Construct FI | Binder V (Table | CD28ζ | IRES C |
| | | 10, #40) | | |
| 150 | Construct FJ | Binder W (Table | CD28ζ | IRES C |
| | | 10, #41) | | |
| 151 | Construct FK | Binder Z (Table | CD28ζ | IRES C |
| | | 10, #42) | | |
| 152 | Construct FL | Binder AA (Table | CD28ζ | IRES C |
| | | 10, #43) | | |
| 157 | Construct FQ | Binder B | CD28ζ | IRES U |
| | | | | (Table 4, |
| | | | | #39) |
| 158 | Construct FR | Binder D | CD28ζ | IRES U |
| 159 | Construct FS | Binder I | CD28ζ | IRES U |
| 160 | Construct FT | Binder M | CD28ζ | IRES U |
| 161 | Construct FU | Binder N | CD28ζ | IRES U |
| 162 | Construct FV | Binder O | CD28ζ | IRES U |
| 164 | Construct FX | Binder Q | CD28ζ | IRES U |
| 165 | Construct FY | Binder R | CD28ζ | IRES U |
| 166 | Construct FZ | Binder S | CD28ζ | IRES U |
| 168 | Construct GB | Binder U | CD28ζ | IRES U |
| 169 | Construct GC | Binder V | CD28ζ | IRES V |
| | | | | (Table 4, |
| | | | | #40) |
| 170 | Construct GD | Binder Z | CD28ζ | IRES V |
| 173 | Construct GG | Binder B | CD28ζ | IRES V |
| 174 | Construct GH | Binder D | CD28ζ | IRES V |
| 175 | Construct GI | Binder I | CD28ζ | IRES V |
| 176 | Construct GJ | Binder M | CD28ζ | IRES V |
| 177 | Construct GK | Binder N | CD28ζ | IRES V |
| 178 | Construct GL | Binder O | CD28ζ | IRES V |
| 180 | Construct GN | Binder Q | CD28ζ | IRES V |
| 181 | Construct GO | Binder R | CD28ζ | IRES V |
| 182 | Construct GP | Binder S | CD28ζ | IRES V |
| 184 | Construct GR | Binder U | CD28ζ | IRES V |
| 185 | Construct GS | Binder W | CD28ζ | IRES V |
| 186 | Construct GT | Binder AA | CD28ζ | IRES V |

### Example 10: oCAR Expression of Codon Optimized or Other Altered Anti-CD19 CARs in Cynomolgus and Human T Cells

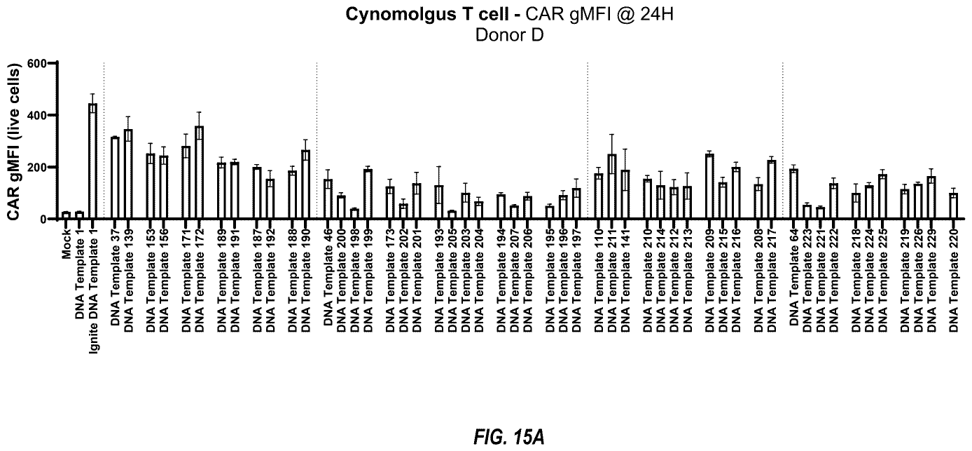

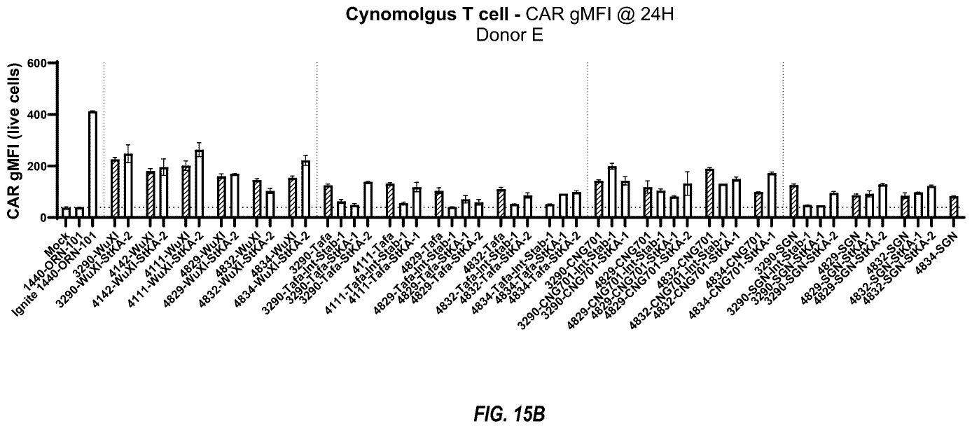

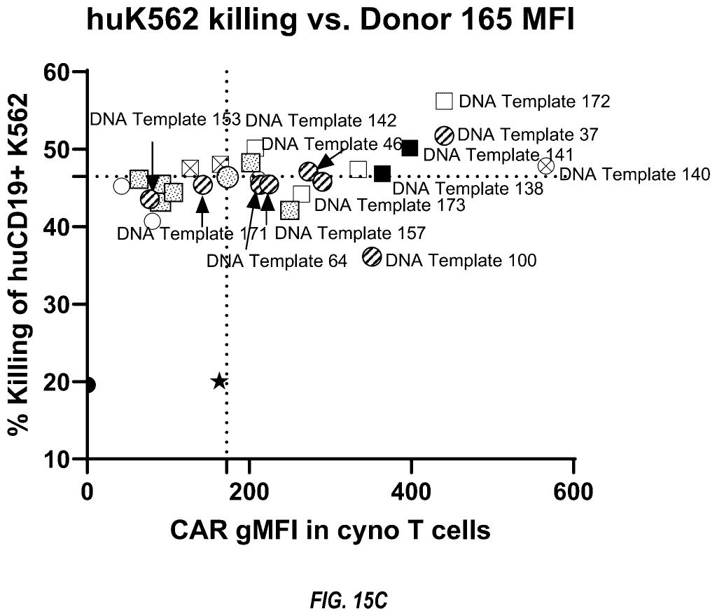

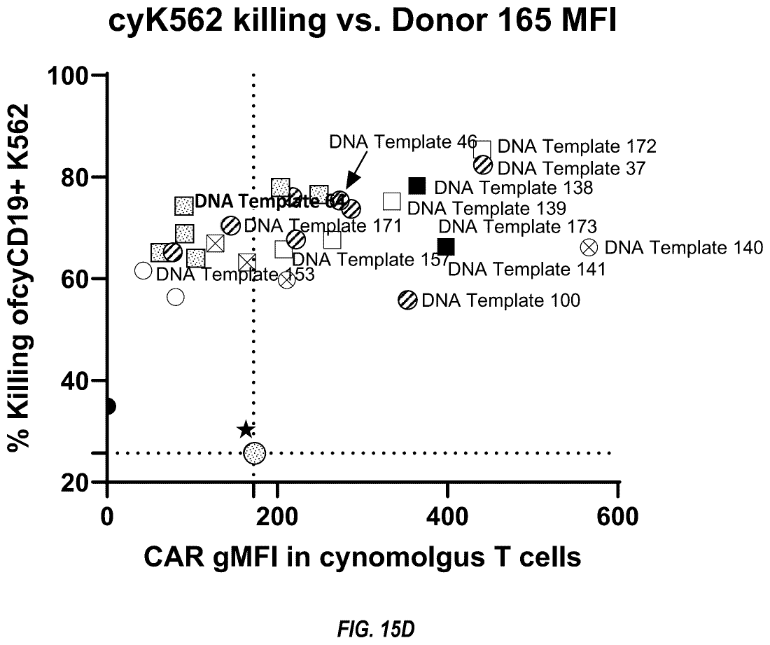

54 circular RNAs encoding anti-CD19 chimeric antigen receptor (CAR) candidates were tested to express in vitro. Cynomolgus T cells from two donors (e.g., Donor D and Donor E) were activated for 3 days with anti-CD2, anti-CD3 and anti-CD28 beads, and allowed to rest for 24 hours. Concurrently, engineered circular RNA constructs were designed to encode a CD19-CD28ζ CAR. For comparison purposes, “mock” or control circular RNAs encoded a HER2. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs comprise different internal ribosome entry sites (IRES) that have undergone different engineering strategies. CAR sequences tested comprise of binders A, B, D, I, L, N, AB-AG as shown in Table TT. DNA templates (e.g., DNA Templates from Table TT) comprising alterations including codon optimizations, lysine to arginine mutations, and (e.g., additions of one or more G4S linker sequences (SEQ ID NO: 1)). Circular RNAs were formulated with Formula A to form oRNA-LNPs (Formulation A). Cynomolgus donor T cells were transfected with the circular RNAs at 200 ng per 0.1×10⁶ T cell to form CAR-T cells (e.g., see FIG. 15A-15B). As a control, “Mock” T cells not transfected with circular RNAs were analyzed. 24 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at 24 hours post transfection. CAR expression intensity as detected from the soluble CD19 detection reagent is shown in FIG. 15A-15B for the cynomolgus T cells tested.

27 circular RNAs encoding anti-CD19 chimeric antigen receptor (CAR) candidates selected from the 54 circular RNAs were tested to express in vitro. Human T cells from one donor (e.g., Donor were cultured for one day, activated with a soluble anti-CD3 and CD28 cocktail for 3 days, and allowed to rest for 24 hours. Human donor T cells were transfected with the circular RNAs at 84 ng per 0.1×10⁶ T cell to form CAR-T cells. (FIG. 15C). As a control, “Mock” T cells not transfected with circular RNAs were analyzed. 24 and 72 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at one or more timepoints from 24 hours post transfection. Durability of CAR expression was measured by calculating total expression over 72 hours post transfection (e.g., via area under the curve (AUC) of anti-CD19 CAR expression graph). Decay of CAR expression at 72 hours was calculated by determining fold change of geometric mean fluorescent intensity (gMFI) between 72 and 24 hours post transfection (i.e., [gMFI at 72 hours]/[gMFI at 24 hours]). The decay and fold changes are shown in FIGS. 15C-15D for human T cells and cynomolgus T cells respectively.

TABLE ΨΨ

| DNA | | | | |
|---|---|---|---|---|
| Template | Construct Number | | Costim | |
| # | from Table 5 | Binder (s) | domain | IRES |
| 1 | Construct Q | Binder X | CD28ζ | IRES A |
| 46 | Construct BJ | Binder B | CD28ζ | IRES C |
| 64 | Construct CB | Binder D | CD28ζ | IRES C |
| 110 | Construct DV | Binder I | CD28ζ | IRES C |
| 141 | Construct FA | Binder N | CD28ζ | IRES C |
| 173 | Construct GG | Binder B | CD28ζ | IRES V |
| 193 | Construct HA | Binder B | CD28ζ | IRES Z |
| | | | | (Table 4, |
| | | | | #43) |
| 194 | Construct HB | Binder B | CD28ζ | IRES W |
| | | | | (Table 4, |
| | | | | #41) |
| 195 | Construct HC | Binder AB (Table | CD28ζ | IRES X |
| | | 10, #44) | | (Table 4, |
| | | | | #42) |
| 196 | Construct HD | Binder AC (Table | CD28ζ | IRES X |
| | | 10, #45) | | |
| 197 | Construct HE | Binder AD (Table | CD28ζ | IRES X |
| | | 10, #46) | | |
| 198 | Construct HF | Binder AC | CD28ζ | IRES C |
| 199 | Construct HG | Binder AD | CD28ζ | IRES C |
| 200 | Construct HH | Binder AB | CD28ζ | IRES C |
| 201 | Construct HI | Binder AC | CD28ζ | IRES V |
| 202 | Construct HJ | Binder AB | CD28ζ | IRES V |
| 203 | Construct HK | Binder AC | CD28ζ | IRES Z |
| 204 | Construct HL | Binder AD | CD28ζ | IRES Z |
| 205 | Construct HM | Binder AB | CD28ζ | IRES Z |
| 206 | Construct HN | Binder AD | CD28ζ | IRES W |
| 207 | Construct HO | Binder AB | CD28ζ | IRES W |
| 208 | Construct HP | Binder I | CD28ζ | IRES X |
| 209 | Construct HQ | Binder I | CD28ζ | IRES W |
| 210 | Construct HR | Binder I | CD28ζ | IRES Z |
| 211 | Construct HS | Binder M | CD28ζ | IRES C |
| 212 | Construct HT | Binder N | CD28ζ | IRES Z |
| 213 | Construct HU | Binder O | CD28ζ | IRES Z |
| 214 | Construct HV | Binder M | CD28ζ | IRES Z |
| 215 | Construct HW | Binder M | CD28ζ | IRES W |
| 216 | Construct HX | Binder N | CD28ζ | IRES W |
| 217 | Construct HY | Binder N | CD28ζ | IRES X |
| 218 | Construct HZ | Binder D | CD28ζ | IRES Z |
| 219 | Construct IA | Binder D | CD28ζ | IRES W |
| 220 | Construct IB | Binder D | CD28ζ | IRES X |
| 221 | Construct IC | Binder AE (Table | CD28ζ | IRES C |
| | | 10, #47) | | |
| 221 | Construct ID | Binder AF (Table | CD28ζ | IRES C |
| | | 10, #48) | | |
| 223 | Construct IE | Binder AG (Table | CD28ζ | IRES C |
| | | 10, #49) | | |
| 224 | Construct IF | Binder AE | CD28ζ | IRES Z |
| 225 | Construct IG | Binder AF | CD28ζ | IRES Z |
| 226 | Construct IH | Binder AE | CD28ζ | IRES W |
| 229 | Construct II | Binder AF | CD28ζ | IRES W |
| 227 | Construct IJ | Binder HER2 | CD282 | IRES A |

### Example 11: oCAR Expression of Codon Optimized or Other Altered Anti-CD19 CARs in Cynomolgus and Human T cells

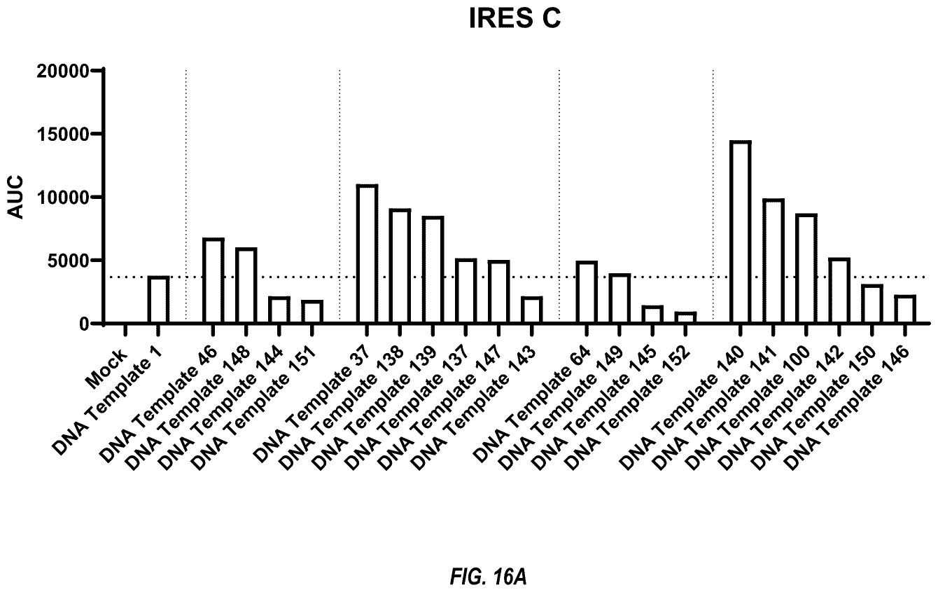

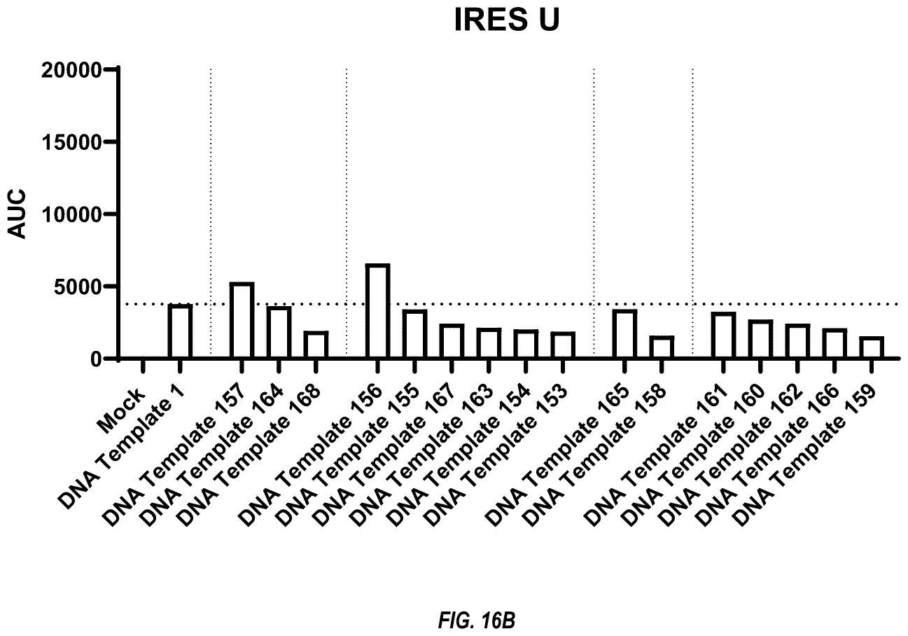

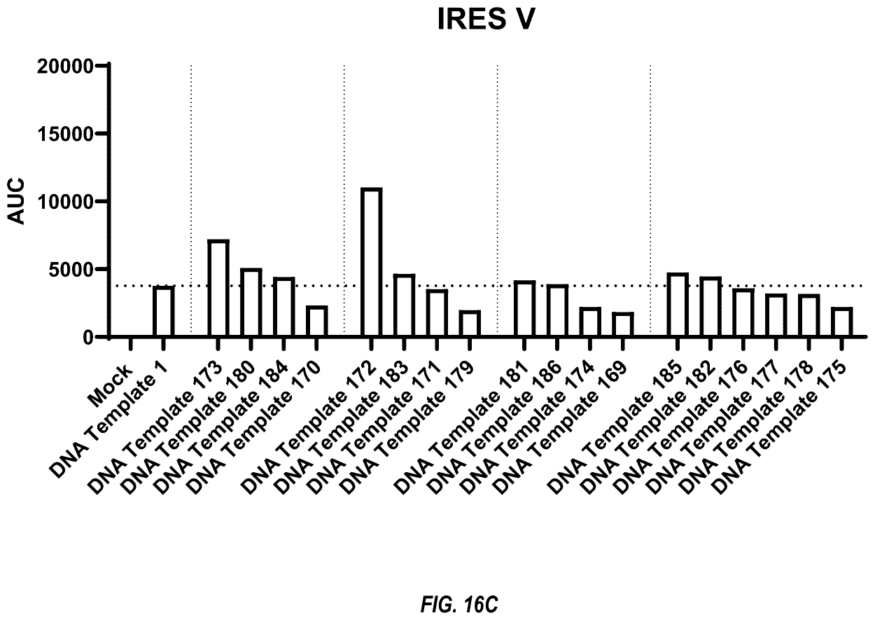

54 circular RNAs encoding anti-CD19 chimeric antigen receptor (CAR) candidates were tested to express in vitro. Cynomolgus T cells from one donor were activated for 3 days with anti-CD2, anti-CD3 and anti-CD28 beads, and allowed to rest for 24 hours. Concurrently, engineered circular RNA constructs were designed to encode a CD19-CD28 CAR. For comparison purposes, “mock” or control circular RNAs encoded a HER2. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs comprise different internal ribosome entry sites (IRES) that have undergone different engineering strategies and originate from Caprine kobuvirus, Hunnivirus, Picornavirus, Coxsackievirus, Enterovirus, or Echovirus. CAR sequences tested comprise of A, B, D, I-Z, AA as shown in Table ωω. DNA templates (e.g., DNA Templates 37, 46, 64, 100, 137-186 from Table ωω) comprising alterations including codon optimizations, lysine to arginine mutations, and (e.g., additions of one or more G4S linker sequences (SEQ ID NO: 1)). Circular RNAs were formulated to form oRNA-LNPs (Formulation A). Cynomolgus donor T cells were transfected with the circular RNAs at 200 ng per 0.1×10⁶ T cell to form CAR-T cells. (FIGS. 16A-16C). As a control, “Mock” T cells not transfected with circular RNAs were analyzed. 24 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at 24 and 72 hours post transfection. The area under the curve for CAR expression intensity as detected from the soluble CD19 detection reagent across these two timepoints is shown in FIGS. 16A-16C for the cynomolgus T cells tested.

### Example 12: oCAR Expression of Codon Optimized or Other Altered Anti-CD19 CARs in Cynomolgus and Human T Cells

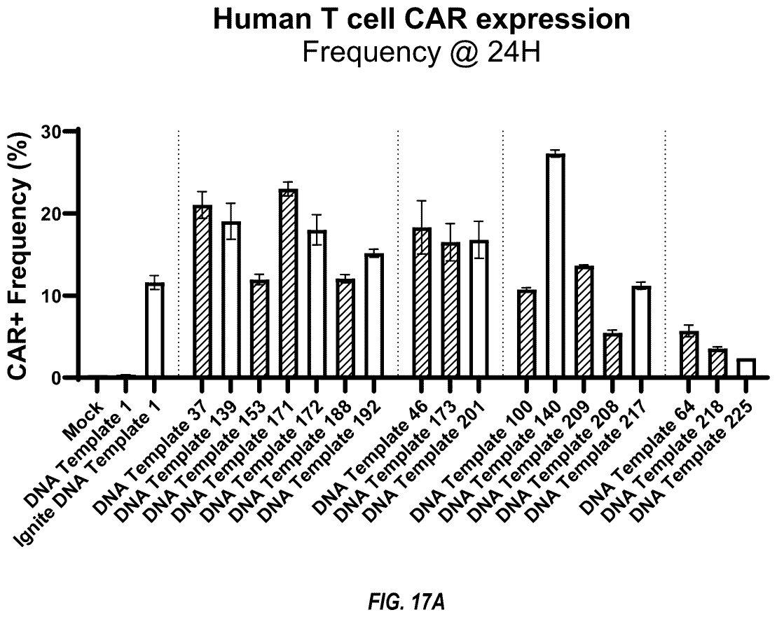

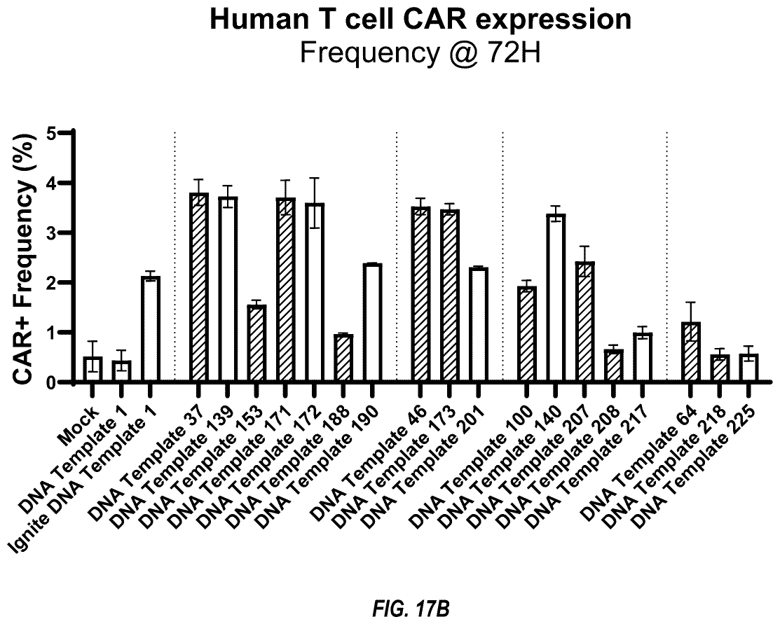

19 circular RNAs encoding anti-CD19 chimeric antigen receptor (CAR) candidates selected from the 52 circular RNAs of Example 10 were tested to express in vitro. DNA templates used to engineer the circular RNAs comprised DNA Templates 1, 37, 46, 64, 110, 139, 153, 171-173, 188, 190, 201, 207-208, 210, 216-217, 224 from Table ‘P’P. Human T cells from one donor were cultured for 1 day, activated with a soluble anti-CD3 and anti-CD28 solution for 3 days, and allowed to rest for 24 hours. The circular RNAs were formulated into LNPs using the methods in Example 10 (i.e., using Formulation A) to produce oRNA-LNPs. Human donor T cells were transfected with the circular RNAs at 100 ng per 0.1×10⁶ T cell to form CAR-T cells. (FIGS. 17A-17B). As a control, “Mock” T cells not transfected with circular RNAs were analyzed. 24 and 72 hours after transfection, CAR-T cells were counted and assessed for CD19CAR expression using commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 CAR expression was assessed using fluorescence activated cell sorting (FACS) and gating flow cytometry methods known in the art at one or more timepoints from 24 hours post transfection. CAR expression frequency as detected from the soluble CD19 detection reagent is shown in FIGS. 17A-17B for the human T cells tested.

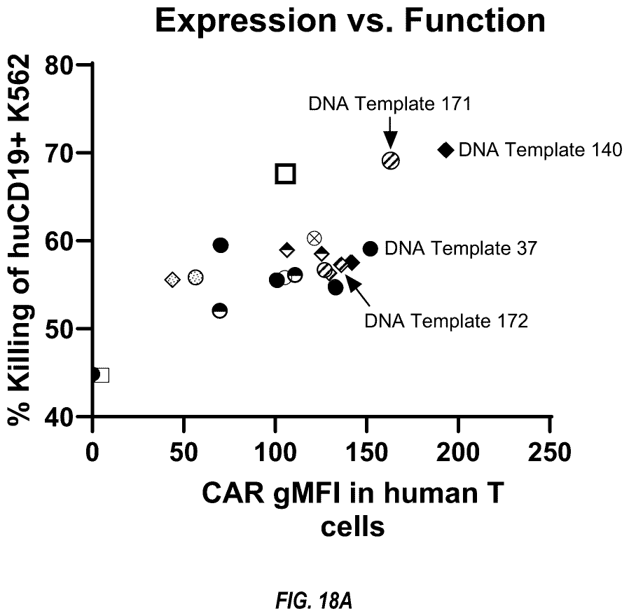

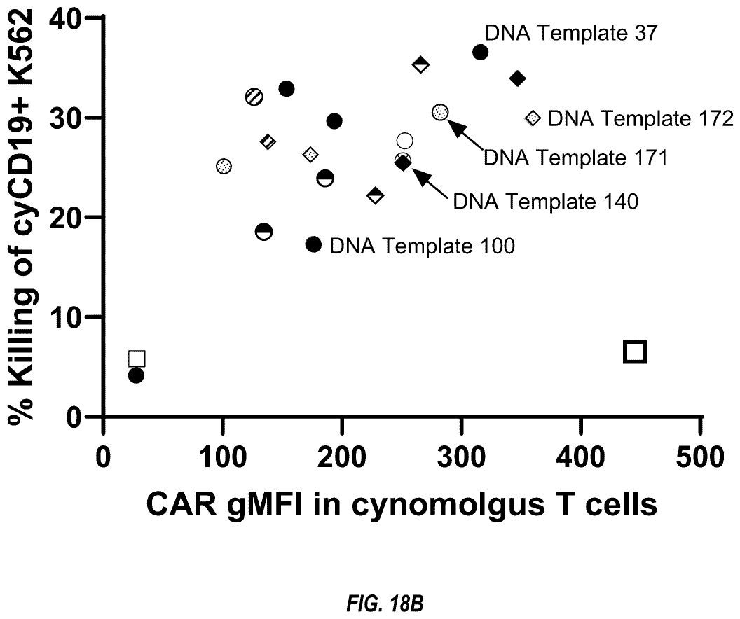

24 hours post transfection, the oCAR-T cells generated from the 18 circular oRNAs encoding anti-CD19 CAR sequences were co-cultured with a tumor cell line (e.g. K562) engineered to overexpress either soluble human CD19 (huCD19) protein or soluble cynomolgus CD19 (cyCD19) protein at a 1:1 or 1:3 E:T, respectively. Cytotoxicity of CD19 targeted killing against huCD19+K562 and cyCD19+K562 was studied via FACS after 48 hours of continuous co-culture. Resulting cytotoxicity levels are shown in FIG. 18A (human T cells) and FIG. 18B.

### Example 13: oCAR Expression of Codon Optimized or Other Altered Anti-CD19 CARs in Cynomolgus and Human T Cells

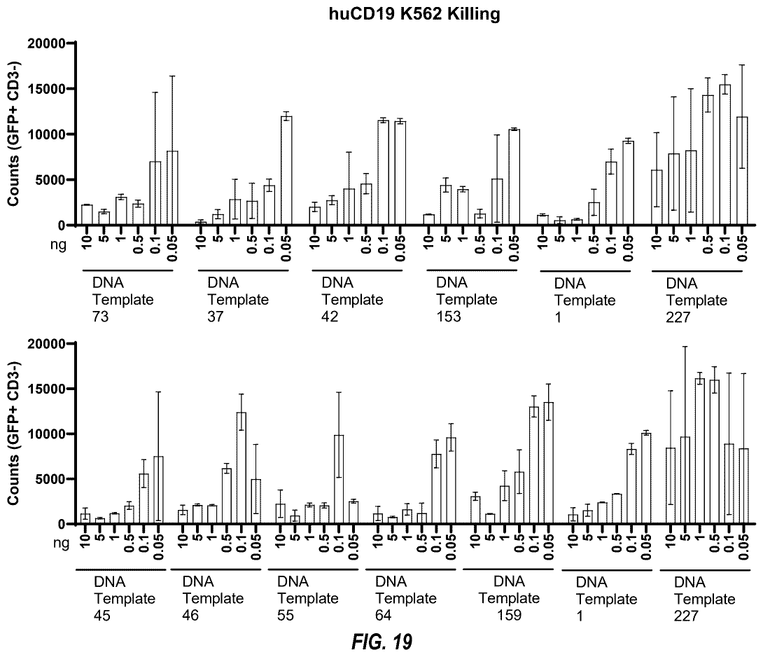

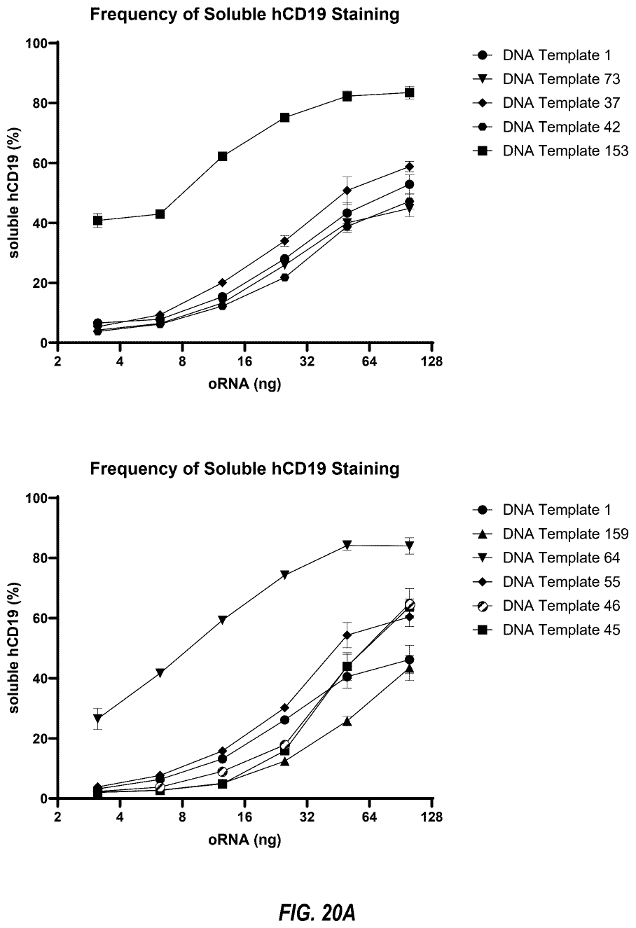

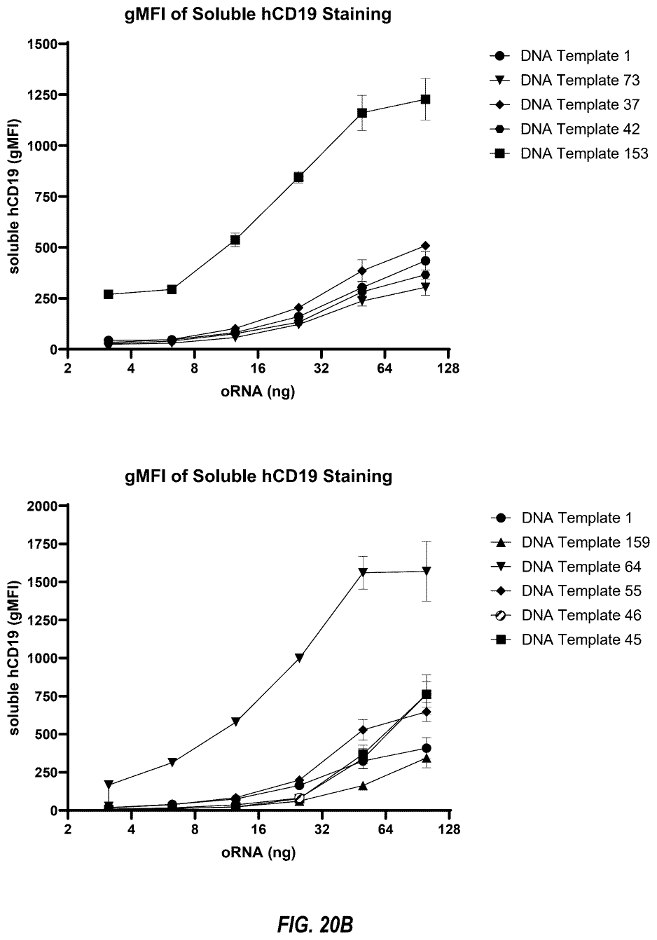

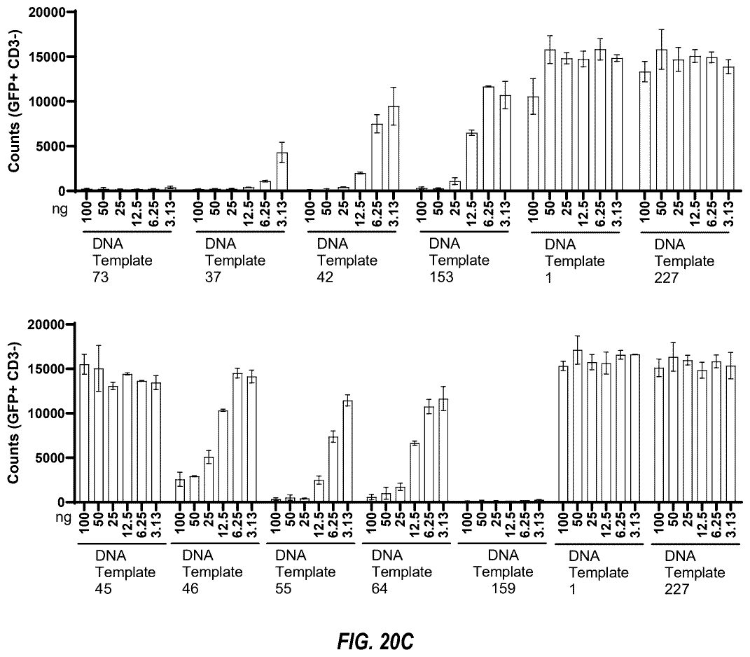

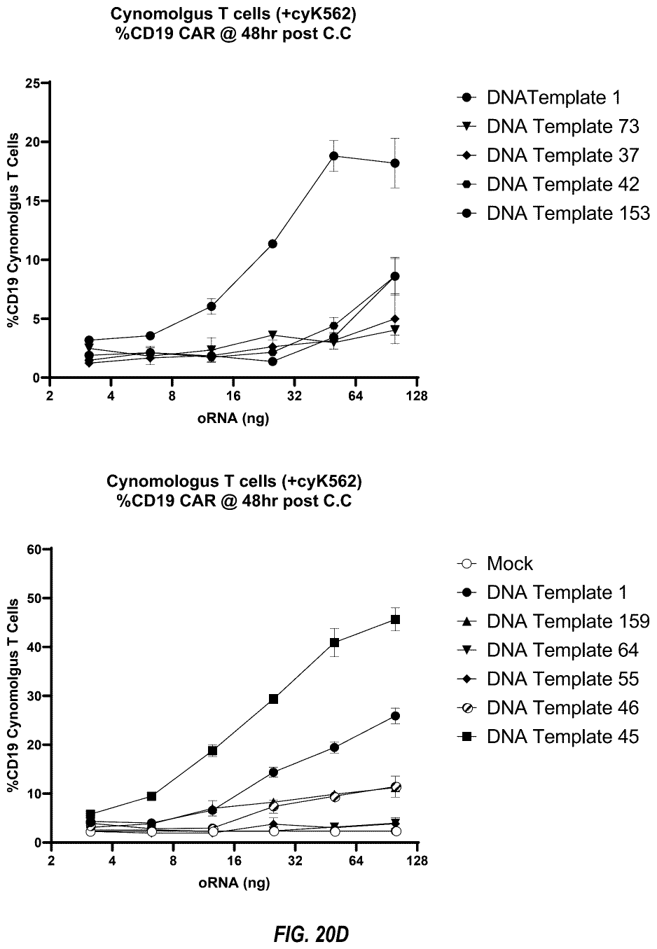

12 circular RNAs encoding anti-CD19 chimeric antigen receptor (CAR) candidates were tested to express in vitro. Human and cynomolgus T cells were activated for 3 days with anti-CD3 and anti-CD28 solution and allowed to rest for 24 hours. Concurrently, engineered circular RNA constructs were designed to encode a CD19-CD28ζ or HER2 (positive control) chimeric antigen receptor (CAR). For comparison purposes, “mock” T cells were not transfected. The circular RNAs were developed from an in vitro translation (IVT) reaction of DNA comprising a T7 polymerase promoter, permuted Anabaena intron exon segments, internal ribosome entry site (IRES), internal spacers, optionally internal homology arms, and a Xlab restriction site. The circular RNAs were engineered to comprise an internal ribosome entry site (IRES). Anti-CD19 CAR sequences used comprised Binder B-I, E, or X as shown in Table W. DNA templates comprised of sequences from the table below. Circular RNAs were formulated with LNPs to form oRNA-LNPs (Formulation A). Human donor T cells were transfected with the oRNA-LNPs at 10 ng, 5 ng, Ing, 0.5 ng, 0.1 ng, or 0.05 ng per 0.1×10⁶ T cells to form CAR-T cells (FIG. 19). Cynomolgus donor T cells were transfected with the oRNA-LNPs at 100 ng, 50 ng, 25 ng, 12. ng, 6.25 ng, or 3.125 ng per 0.1×10⁶ T cells to form CAR-T cells (FIGS. 20A-20C, 20D). As a control, “Mock” T cells not transfected with oRNA-LNPs were analyzed. T cells were then allowed to rest for 24 hours after transfection. 24 hours after transfection, the CAR-T cells were counted and assessed for CD19 CAR expression. Resulting oCAR-T cells were given a commercially available biotinylated soluble CD19 detection reagent (e.g., from Miltenyi Biotec, Bergisch Gladbach) and Streptavidin conjugated to R-phycoerythrin (PE) fluorophores (e.g., from Biolegend, San Diego). Anti-CD19 expression was assessed using fluorescence activated cell sorting (FACS) and gating flow cytometry methods known in the art at one or more timepoints from 24 hours post transfection. CAR expression and intensity as depicted from the soluble CD19 detection reagent is shown in FIGS. 20A-20B for the cynomolgus T cells tested.

Human oCAR-T cells were co-cultured with targeted humanized CD19 (K562.huCD19) or humanized cynomolgus CD19 (K562.cyCD19) cells at selected E:T ratio (i.e., 1:1 ratio) 24-hours following transfection of the oRNA-LNPs to the donor T cells. 48 hours later, cytotoxicity of CD19 targeted killing on K562.cyCD19 (FIG. 20D) was studied via FACS. Anti-CD19 CAR expression was assessed using fluorescence activated cell sorting (FACS) and gating flow cytometry methods known in the art at one or more timepoints from 48 hours post co-culture with K562.cyCD19 (FIG. 20C).

Cynomolgus oCAR-T cells were co-cultured with targeted humanized cynomolgus CD19 (K562.cyCD19) cells at selected E:T ratio (i.e., 1:1 ratio) 24-hours following transfection of the oRNA-LNPs to the donor T cells. 48 hours later, cytotoxicity of CD19 targeted killing on K562.cyCD19 (FIG. 20D) was studied via FACS. Anti-CD19 CAR expression was assessed using fluorescence activated cell sorting (FACS) and gating flow cytometry methods known in the art at one or more timepoints from 48 hours post co-culture with K562.cyCD19 (FIG. 20D).

TABLE ψψ

| DNA | Construct Number | | Costim | |
|---|---|---|---|---|
| Template # | from Table 5 | Binder (s) | domain | IRES |
| 3 | Construct S | Binder X | CD28ζ | IRES C |
| 45 | Construct BI | Binder B | CD28ζ | IRES B |
| 46 | Construct BJ | Binder B | CD28ζ | IRES C |
| 55 | Construct BS | Binder C | CD28ζ | IRES C |
| 64 | Construct CB | Binder D | CD28ζ | IRES C |
| 73 | Construct CK | Binder E | CD28ζ | IRES C |
| 159 | Construct FS | Binder I | CD28ζ | IRES U |
| 227 | Construct IJ | Binder HER2 | CD28ζ | IRES A |
| 228 | Construct IK | Binder X | CD28ζ | IRES A |

### Example 14: LNP Delivery of oRNA CAR Payload to T and NK Cells—Non-Human Primates

LNP formulations were prepared as described above. Exemplary LNPs comprised circular RNA encoding anti-CD19 CAR as set forth in Table 10 at 10-97, IRES set forth in Table 4 at 4-18, CD28ζ (set forth above, miR-122 binding site after the stop codon, and formulated as shown in the table below. Each formulation was administered to Cynomolgus monkeys on Day 1, Day 15 and Day 29 via 60-min intravenous infusion into an appropriate peripheral vein using an infusion pump at a dose level of either 0.3 mg/kg or 0.1 mg/kg of oRNA (dose volume of 5 mL/kg; concentration 0.06 mg/mL or 0.02 mg/mL, respectively). Groups dosed with 0.3 mg/kg of LNP formulation were administered dexamethasone prior to LNP infusion; dexamethasone was administered IM at a dose of 1 mg/kg. Each LNP formulation and control was dosed in the following number of animals: F-A 0.3 mg/kg-3; F-B 0.3 mg/kg-3; F-B 0.1 mg/kg-3. Blood samples were collected at baseline, 24 hours, 48 hours, 72 hours, and 168 hours post dosing for dose 1 and dose 2, and at baseline and 24 hours post dosing for dose 3. Whole blood was processed to isolate Peripheral Blood Mononuclear Cells (PBMC) and cryopreserved for later flow cytometry analysis.

oRNA CAR Formulations

| | | | | | N:P/ | Size | | |
|---|---|---|---|---|---|---|---|---|
| Form | IL | PL | PEG | Sterol | Buffer | (nm) | PDI | ee % |
| F-A* | AX-6 | ESM | PEG2k-DMG | Chol. | 8 | 86.3 | 0.06 | 92.1 |
| | 33 | 40 | 2 | 25 | Y | | | |
| F-B# | AX-6 | ESM | PEG2k-DMG | Chol. | 8 | 85.7 | 0.07 | 92.4 |
| | 33 | 40 | 2 | 25 | Y | | | |

*non-NHP cross-reactive anti-CD19 CAR ORNA payload (negative control)
#anti-CD19 CAR ORNA payload
Buffer X: 25 mM Sodium Acetate, pH 5.0; Buffer Y: 50 mM Citrate, pH 4.0; SM = egg sphingomyelin; PEG2k-DMG = 1,2-dimyristoyl-rac-glycero-3-methoxypolyethylene glycol-2000; Chol = cholesterol

Cryopreserved PBMC were thawed and washed with complete RPMI supplemented with 10% heat-inactivated FBS (GIBCO 72400-047 and A38400-01) before incubating with plain RPMI with 50 U/mL Benzonase (EMD 7066-10KUN) at 37° C. for 15 minutes. Cells were then centrifuged, resuspended in cRPMI and counted (Cellaca MX, PerkinElmer MX-AOPI). Cells were plated at 5 million cells per well in a 96-well round bottom plate (Costar 3799) and stained for flow cytometry. Briefly, cells were stained in 1×PBS with Live/Dead Fixable Aqua (Invitrogen L34966) at 1:1000 for 20 minutes at room temperature. Cells were then washed twice with Cell Staining Buffer (BioLegend 420201) and incubated with Fc block for 5 minutes at 4° C. and surface antibody stains either in full or FMO (fluorescence minus one) master mixes were added on top of the Fc Block for an additional 30 minutes at 4° C. Cells were then washed three times with Cell Staining Buffer, and resuspended in fixative (BD 554655) then incubated at 4° C. for 20 minutes. Cells were washed twice with 1×PBS and filtered through a 30-40 μm filter (Pall 8027) and 20 μL of counting beads (CountBright Absolute Counting Beads, ThermoFisher C36950) were added to each sample before acquisition on the cytometer (Sony ID7000 Cell Analyzer: Cat #LE-ID7000C with UV/V/B/YG/R lasers). Single stain controls were made using UltraComp eBeads (ThermoFisher 01-3333-41) and ArC Amine Reactive Compensation Bead Kit (ThermoFisher A10346). Analysis was performed using Flowjo (BD V10.8.1). B cells were identified as Live/Dead (L/D) Aqua-CD3-CD20+.

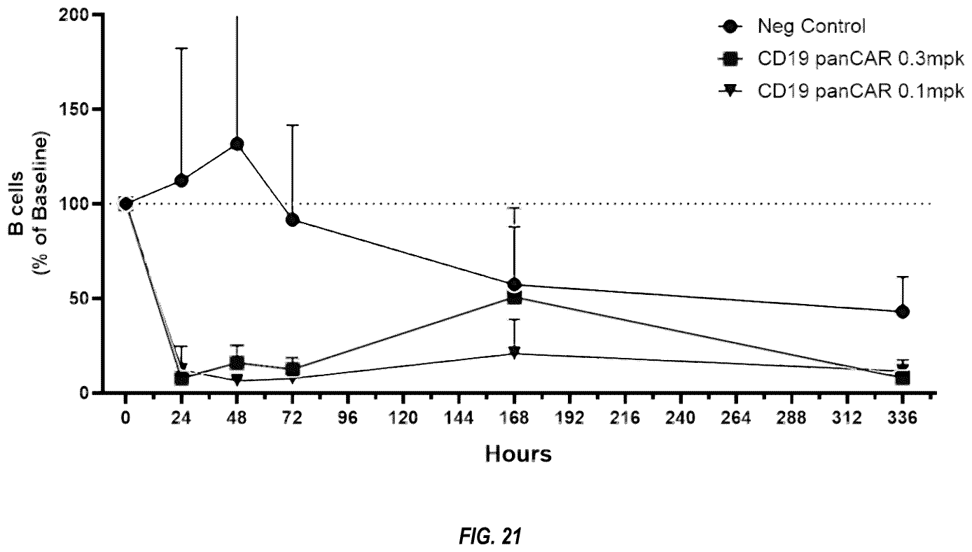

Results are shown in FIG. 21.

### Example 15: B-Cell Depletion in Non-Human Primates

LNP formulations were prepared as described in Example 2B. Thirty minutes prior to all doses, all animals were dosed with Dexamethasone at 1 mg/kg via intramuscular injection. Each formulation was administered to Cynomolgus monkeys on Day 1 and Day 4 via 60-min intravenous infusion into an appropriate peripheral vein using an infusion pump. Dose groups were as follows: Group 1 contained 2 monkeys dosed with PBS; Group 2 contained 5 monkeys dosed with formulation F-S1, containing VHH oRNA, at a dose level of 1.0 mg/kg oRNA (dose volume of 5 mL/kg); Group 3 contained 6 monkeys dosed with formulation F-S2, containing the anti-CD19 panCAR encoding oRNA (anti-CD19 CAR as set forth in Table 10 at 10-97, IRES set forth in Table 4 at 4-18), at a dose level of 1 mg/kg oRNA (dose volume of 5 mL/kg); Group 4 contained 6 monkeys dosed with formulation F-S2 at a dose level of 0.5 mg/kg (dose volume of 5 mL/kg); Group 5 contained 6 monkeys dosed with formulation F-S2 at a dose level of 0.1 mg/kg (dose volume of 5 mL/kg). Blood samples were collected in EDTA at Day −7, Day 1 (pre-dose 1), Day 2, Day 4 (pre-dose 2), Day 5, Day 9, Day 11, Day 18, Day 25, Day 32, Day 39 and Day 56. A subset of test subjects were terminated at Day 7 (Group 1 n=2, Group 2 n=3, Group 3 n=3, Group 4 n=3, Group 5 n=3); the remaining test subjects were terminated at Day 56. 100 μL of whole blood per sample was aliquoted into a deep-well 96-well plate (USA Scientific 1896-2000) and then 2 mL of 1× BD Pharm Lyse (BD Biosciences 555889) was added to the wells and mixed by pipetting; samples were incubated at room temperature for 15 minutes, and were mixed by pipette half-way through the incubation. Cells were centrifuged at 350×g for 5 minutes, and supernatant discarded. Cells were washed with PBS and transferred to a standard 96-well round bottom plate and stained for flow cytometry. Test subject femurs were flushed for collection of bone marrow and spleens and lymph nodes were collected for analysis by flow cytometry. For the lymph nodes, mesenteric lymph nodes were collected and pooled and are designated as ‘mLN’; the draining lymph nodes, designated as ‘dLN’, consist of a pool of the Iliac, Axillary and Inguinal lymph nodes. For spleens and for lymph nodes up to 1 gram of harvested tissue type per animal was dissociated into single cell suspension using the gentleMACS Octo Dissociator with Heaters (Miltenyi 130-096-427) with the Multi Tissue Dissociation Kit I (Miltenyi 130-110-201) per manufacturer's instructions. Dissociated splenocytes or lymph nodes were then passed through a 70 μm filter (Miltenyi 130-098-462) and washed with 1×PBS (ThermoFisher 10010049) containing 2 mM EDTA (ThermoFisher 15575-020), 0.5% BSA (Miltenyi 130-091-376) and 1×+1× Antimycotic/Antibiotic (ThermoFisher 15240-062). Red blood cells were lysed using ACK Lysing Buffer (Thermo Fisher A1049201) and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA+1× Antimycotic/Antibiotic and counted (Cellaca MX, PerkinElmer MX-AOPI). Cells were diluted, plated (5,000,000 per well) in a 96-well round bottom plate (Costar 3799), and stained for flow cytometry. Flushed bone marrow was centrifuged and cells were collected, passed through a 70 μm filter, and washed with 1×PBS+2 mM EDTA+0.5% BSA+1× Antimycotic/Antibiotic. Red blood cells were lysed using ACK Lysing Buffer and washed twice with 1×PBS+2 mM EDTA+0.5% BSA+1× Antimycotic/Antibiotic, passing the cell suspension through an additional 70 μm filter prior to the last wash. Following final wash, cells were resuspended in 1×PBS+2 mM EDTA+0.5% BSA+1× Antimycotic/Antibiotic and counted. Cells were diluted, plated (15 million per well) in a 96-well bottom plate, and stained for flow cytometry. Cells were stained in 1×PBS with Live/Dead Fixable NearIR-780 (Invitrogen L34994) at 1:1000 for 20 min at room temperature. Cells were then washed twice with Cell Staining Buffer (BioLegend 420201) and incubated with Fc block for 5 min at 4° C. and surface antibody stains either in full or FMO (fluorescence minus one) master mixes (panels and dilutions shown below in Tables ZA-ZC) added on top of the Fc Block for an additional 30 min at 4° C. For cells stain with Panel 1 and Panel 2, (Table ZA-ZB), cells were washed twice with Cell Staining Buffer and incubated with Streptavidin-PE (BioLegend 405204) at 4° C. for 15 min. For cells stained with Panel 3 (Table ZC), they proceeded directly to washing and fixing. For all panels, cells were then washed three times with Cell Staining Buffer and fixed with Cytofix (BD 554655) diluted 1:2 in PBS and incubated at 4° C. for 20 min. Cells were washed twice with 1×PBS and filtered through a 30-40 μm filter (Pall 8027) and acquired on cytometer(Sony D7000 Cell Analyzer: Cat #LE-ID7000C with UV/V/B/YG/R lasers). Single stain controls were made using UltraComp eBeads (ThermoFisher 01-3333-41) and ArC Amine Reactive Compensation Bead Kit (ThermoFisher A10346). Analysis was performed using Flowjo (BD V10.8.1). Cells were identified with markers in Tables ZD-ZF and gates for measuring VHH reporter+ or CD19 CAR+ cells were placed so that the negative control would be <0.5%.

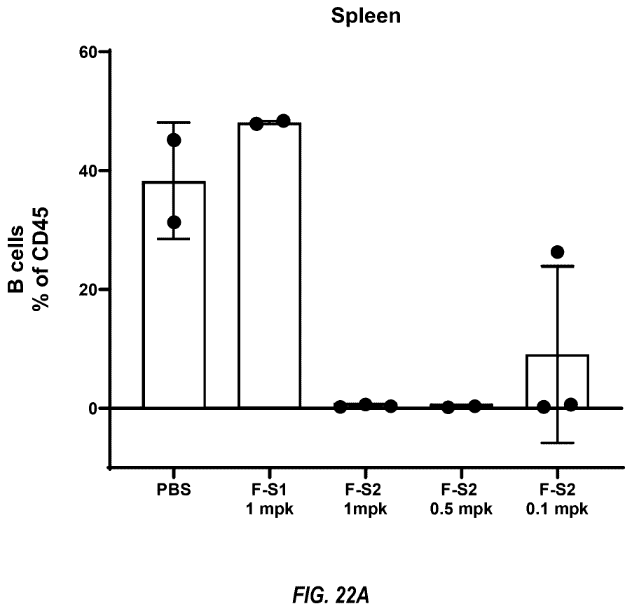

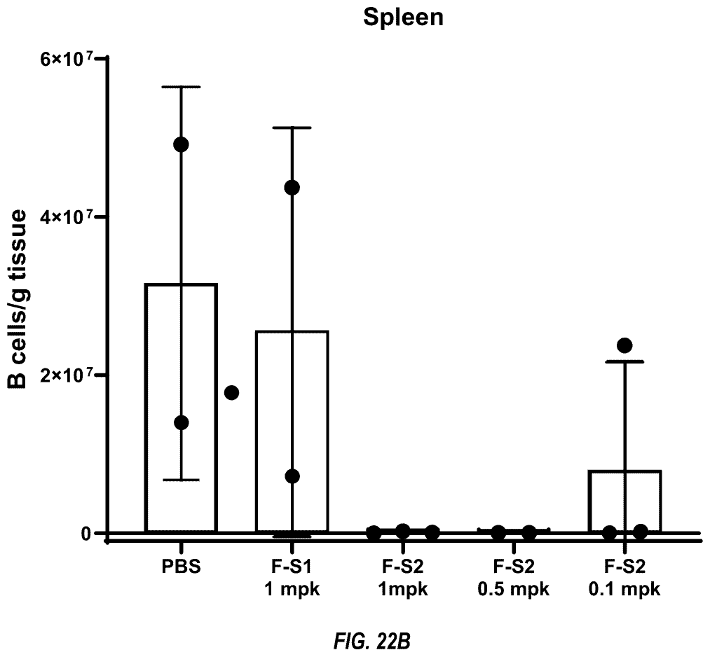

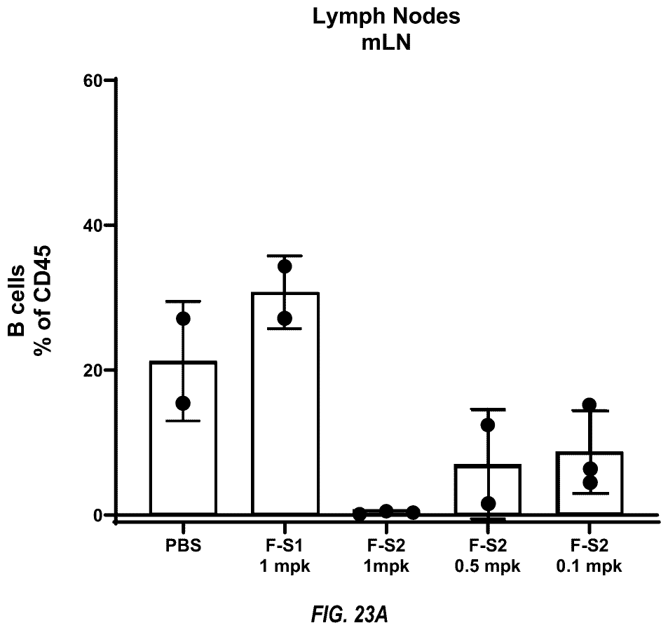

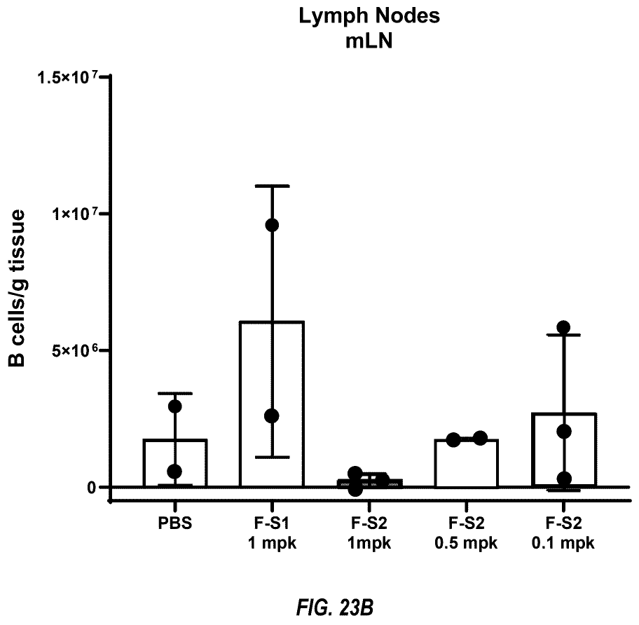

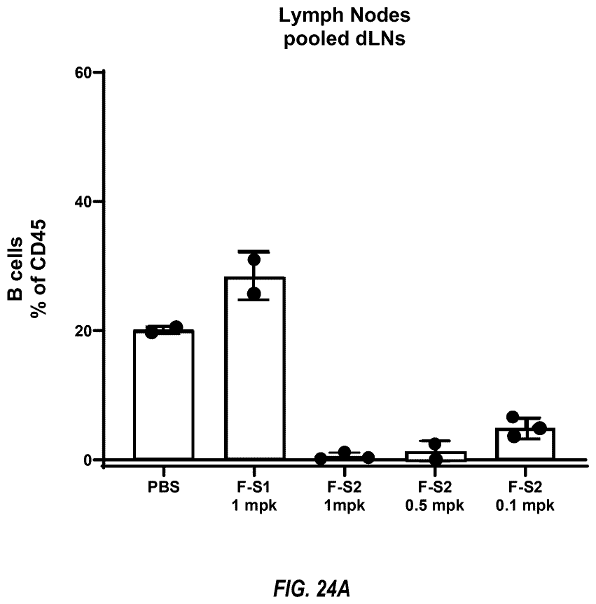

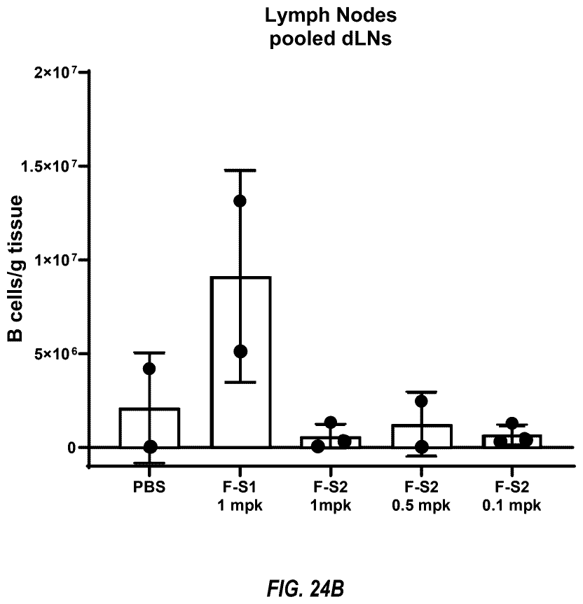

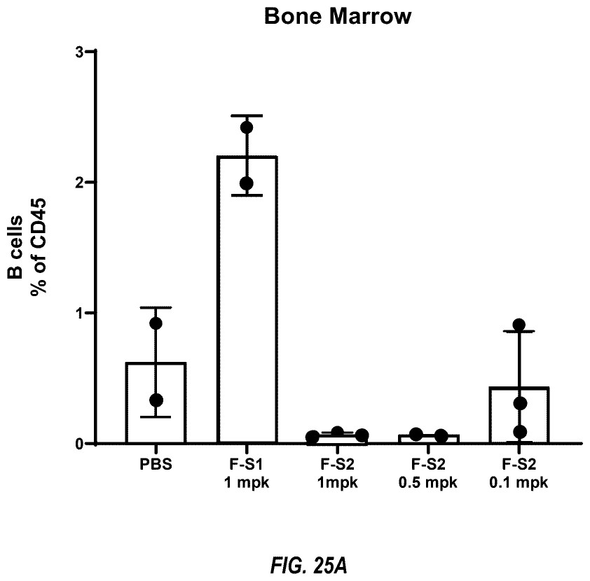

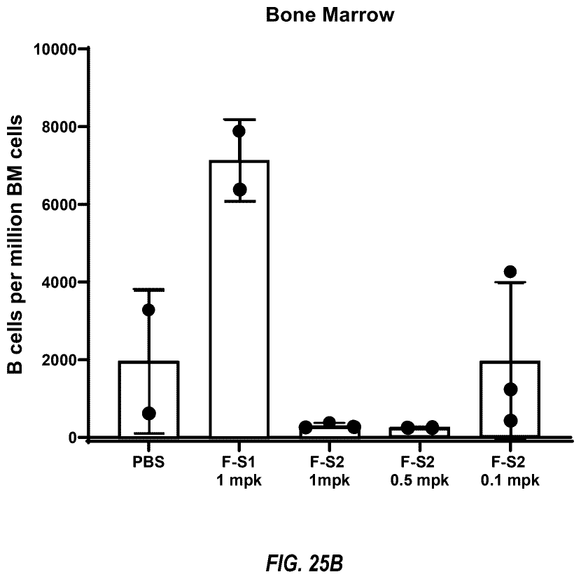

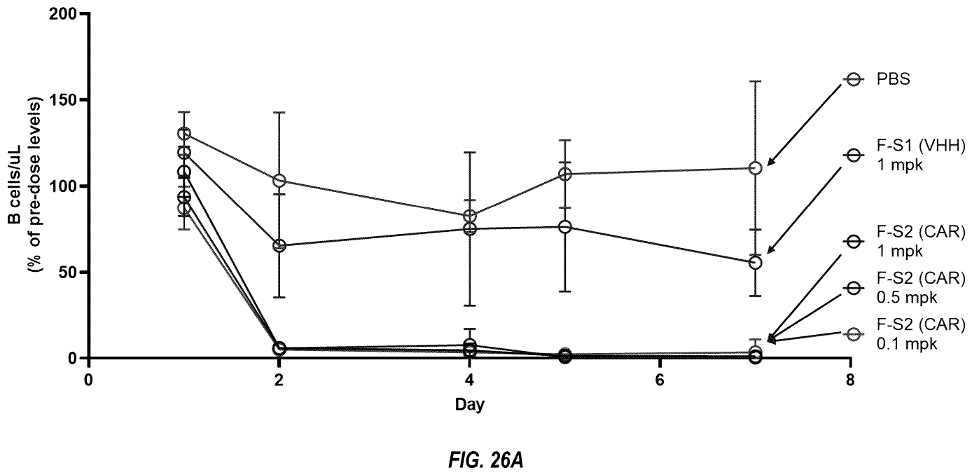

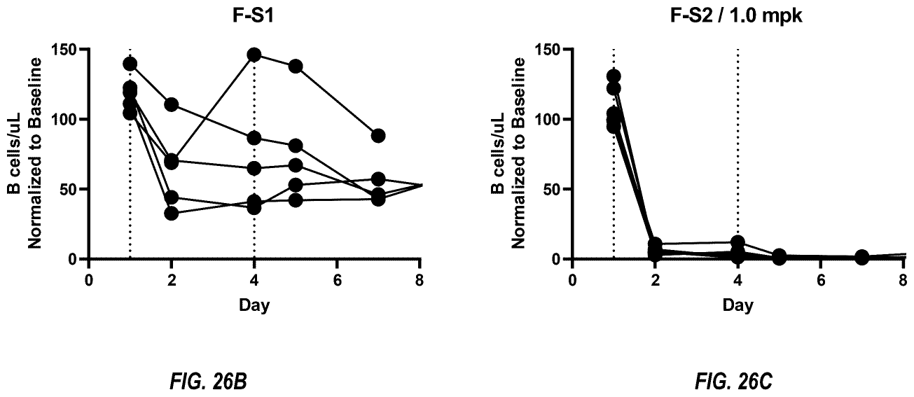

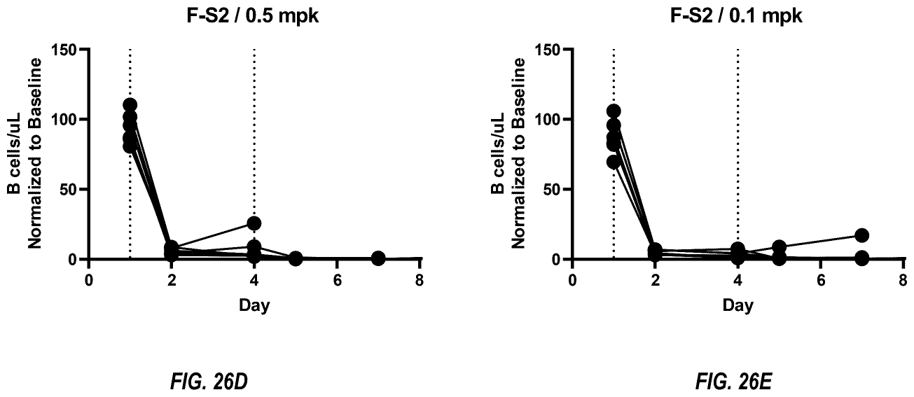

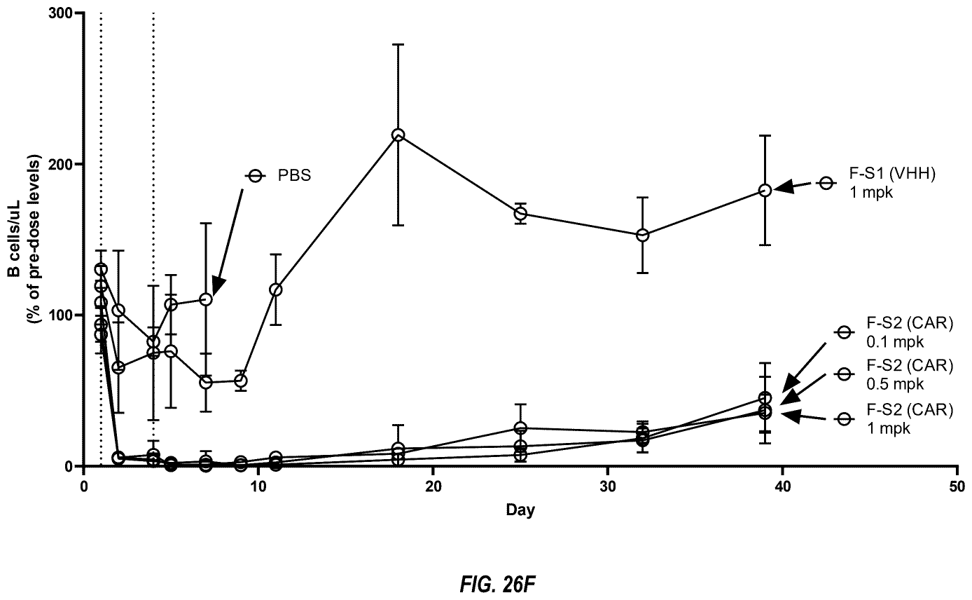

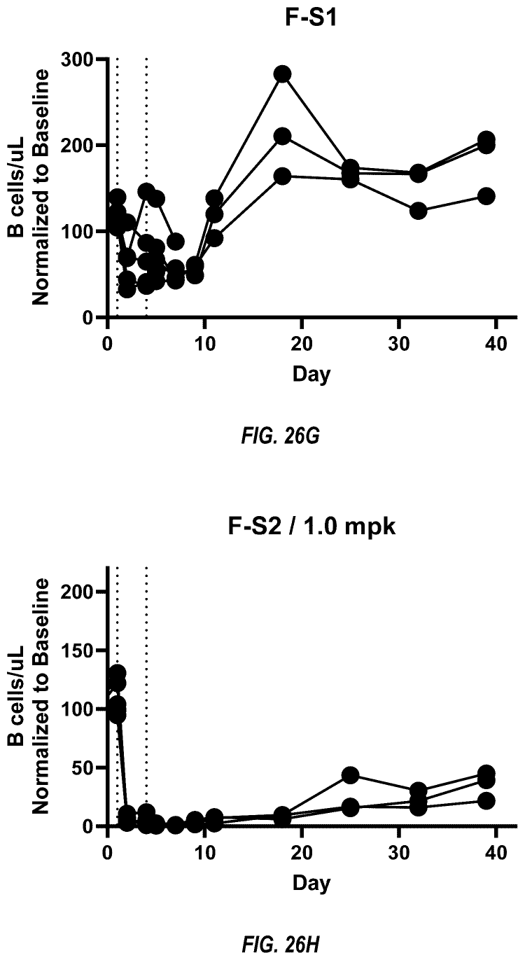

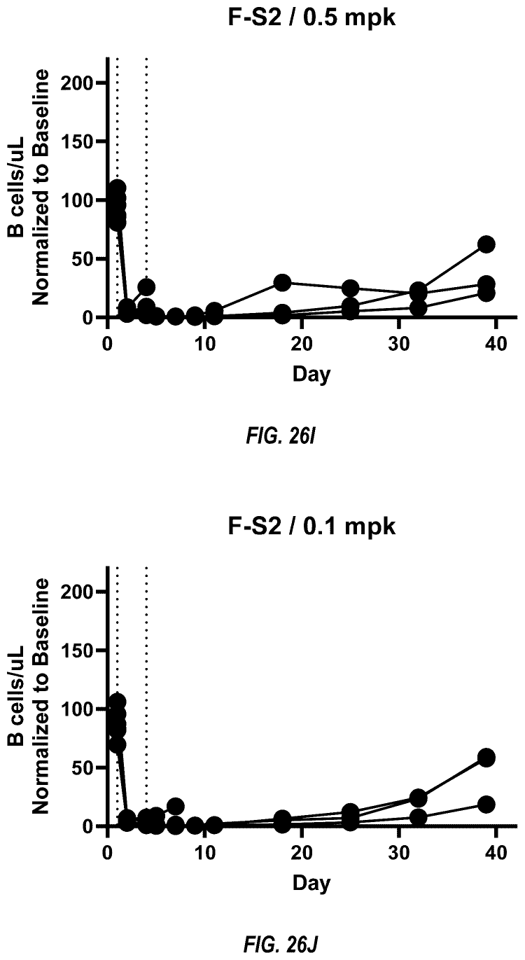

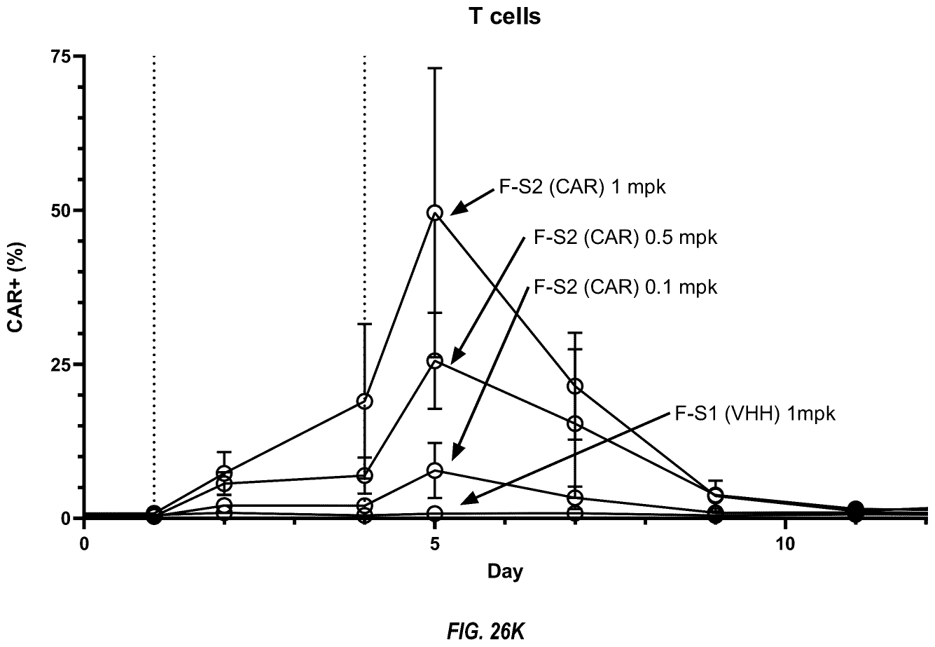

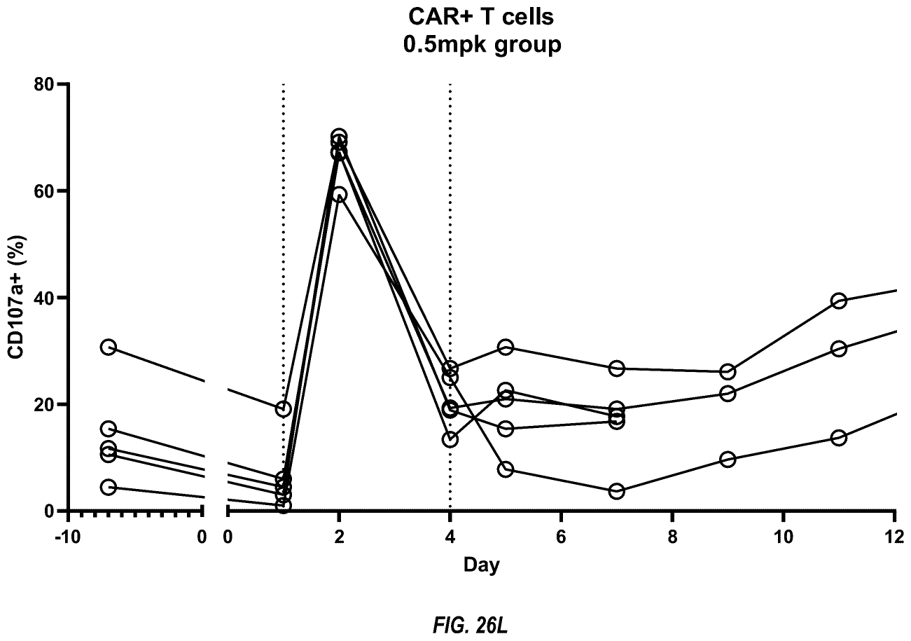

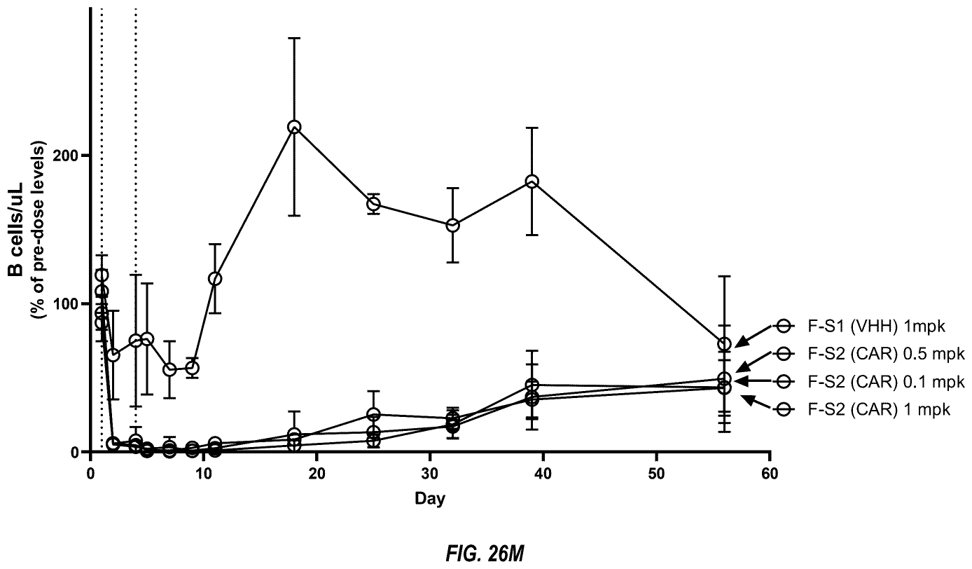

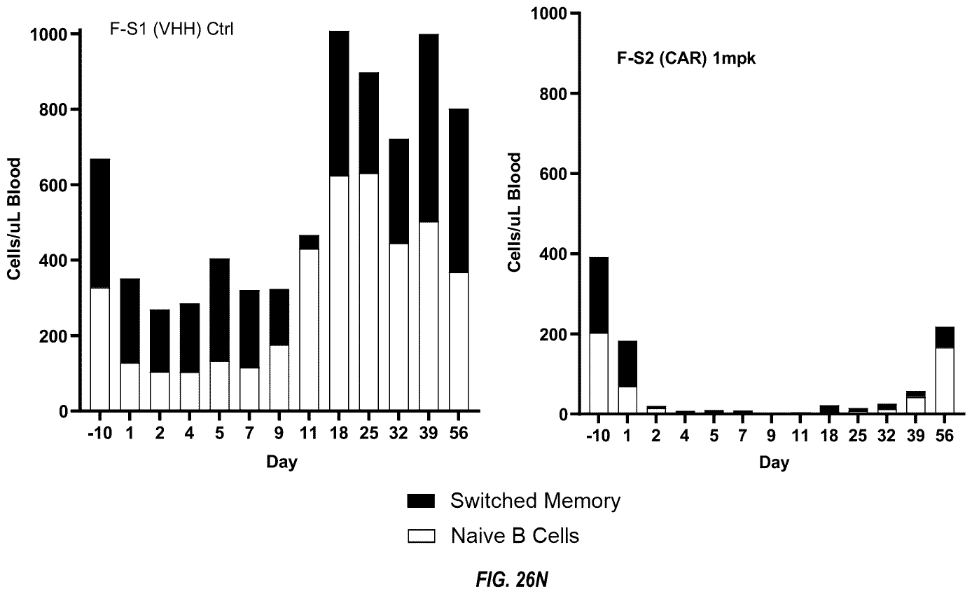

Results are shown in FIGS. 22A through 26J. As shown in FIGS. 26A through 26J, animals dosed with CAR test article F-S2 demonstrated robust B cell depletion at all dose levels, as compared animals dosed with PBS and VHH control F-S1. As shown in FIG. 26F-26J, B cell levels began to rise again around day 10, recovering to about ⅓ of pre-dose levels by day 39 for all groups dosed with F-S2. FIG. 26M shows B cell levels at extended time points. FIG. 26N shows reconstituted B cells (naive B cells and switched memory cells) over time after dosing. FIG. 26K shows CAR expression on T cells. FIG. 26L shows upregulation of an exemplary cytotoxicity marker. Together, these data show that rapid cytotoxic effects occur after the first dose, and the majority of target cell killing occurs after the first dose.

TABLE ZA
Panel 1 T cell phenotyping

| | | | | | | Usage |
|---|---|---|---|---|---|---|
| Staining | | | | | | per test |
| Step: | Target | Fluorophore | Clone | Manufacturer | Cat No. | (dilution) |
| Live/Dead | Viability | Near IR-780 | N/A | Invitrogen | L34994 | 1000 |
| Surface | Super Bright Staining Buffer | | | eBioscience | SB-4401-75 | 20 |
| Primary | VHH | iFluor 488 | 96A3F5 | GenScript | A01862 | 100 |
| | CD19 CAR | Biotin | rCD19-biotin | Miltenyi | 130-129-550 | 100 |
| | CD45 | AF700 | D058-1283 | BD | 561288 | 20 |
| | CD3 | PERCP-Cy5.5 | SP34-2 | BD | 552852 | 10 |
| | CD4 | BV785 | OKT4 | Biolegend | 317442 | 20 |
| | CD8 | BV510 | SK1 | Biolegend | 344732 | 20 |
| | CD25 | BV605 | BC96 | Biolegend | 302632 | 20 |
| | CD127 | PE-Vio615 | MB15-18C9 | Miltenyi | 130-113-411 | 20 |
| | PD1 | PE-Cy7 | EH12.2H7 | Biolegend | 329918 | 20 |
| | TIGIT | APC | MBSA43 | eBioscience | 17-9500-42 | 20 |
| | CD95 | BV421 | DX2 | Biolegend | 305624 | 20 |
| | CD28 | BV650 | CD28.2 | Biolegend | 302946 | 20 |
| Surface | Strepavidin | PE | N/A | Biolegend | 405204 | 200 |
| Secondary | | | | | | |

TABLE ZB
Panel 2 CAR expression on Lymphocyte and myeloid
cell subsets and B cell enumeration.

| | | | | | | Usage |
|---|---|---|---|---|---|---|
| Staining | | | | | | per test |
| Step: | Target | Fluorophore | Clone | Manufacturer | Cat No. | (dilution) |
| Live/Dead | Viability | Near IR-780 | N/A | Invitrogen | L34994 | 1000 |
| Surface | Super Bright Staining Buffer | | | eBioscience | SB-4401-75 | 20 |
| Primary | VHH | iFluor 488 | 96A3F5 | GenScript | A01862 | 100 |
| | CD19 CAR | Biotin | rCD19 | Miltenyi | 130-129-550 | 100 |
| | CD45 | AF700 | D058-1283 | BD | 561288 | 20 |
| | CD20 | PE-Cy7 | 2H7 | BioLegend | 302312 | 20 |
| | HLA-DR | PE-CF594 | G46-6 | BD | 562304 | 100 |
| | CD3 | PERCP Cy5.5 | SP34-2 | BD | 552852 | 10 |
| | CD159a | APC | Z199 | Beckman Coulter | A60797 | 10 |
| | CD11b | BV650 | ICRF44 | Biolegend | 301336 | 20 |
| | CD69 | BV605 | FN50 | Biolegend | 310938 | 20 |
| | CD107a | BV421 | H4A3 | Biolegend | 328626 | 20 |
| | CD163 | BV510 | GHI/61 | Biolegend | 333628 | 20 |
| Surface | Strepavidin | PE | N/A | Biolegend | 405204 | 200 |
| Secondary | | | | | | |

TABLE ZC
Panel 3 B cell phenotyping

| | | | | | | Usage |
|---|---|---|---|---|---|---|
| Staining | | | | | | per test |
| Step: | Target | Fluorophore | Clone | Manufacturer | Cat No. | (dilution) |
| Live/Dead | Viability | Near IR-780 | N/A | Invitrogen | L34994 | 1000 |
| Surface | Super Bright Staining Buffer | | | eBioscience | SB-4401-75 | 20 |
| | IgD | AF488 | Polyclonal (Goat) | Southern Biotech | 2030-30 | 20 |
| | CD38 | APC | OKT10 | Caprico | 100846 | 50 |
| | CD20 | PERCP-Cy5.5 | 2H7 | BioLegend | 302326 | 20 |
| | CD45 | AF700 | D058-1283 | BD | 561288 | 20 |
| | IgM | BV421 | G20-127 | BD | 562618 | 20 |
| | CD21 | BV605 | B-ly4 | BD | 740395 | 20 |
| | CD27 | PE | 0323 | eBio | 12-0279-42 | 20 |
| | CD19 | PE-Cy7 | J3-119 | Beckman Coulter | IM3628U | 4 |

TABLE ZD
Definition of Cell Subsets in NHP
by Flow Cytometry used in Table ZA:

| Cell Type: | Surface Marker Phenotype: |
|---|---|
| T cells (Total) | L/DNIR780−CD45+CD3+ |
| CD4 T cells | L/DNIR780−CD45+CD3+CD4+CD8− |
| CD8 T cells | L/DNIR780−CD45+CD3+CD4−CD8+ |
| Naïve T cells | L/DNIR780−CD45+CD3+CD28+CD95− |
| Central Memory | L/DNIR780−CD45+CD3+CD28+CD95+ |
| T cells | |
| Effector Memory | L/DNIR780−CD45+CD3+CD28−CD95+ |
| T cells | |
| Regulatory | L/DNIR780−CD45+CD3+CD4+CD8−CD127−CD25^{hi} |
| T cells | |
| Exhausted | L/DNIR780−CD45+CD3+PD1^{hi}TIGIT^{h}i |
| T cells | |

TABLE ZE
Definition of Cell Subsets in NHP by Flow Cytometry used in Table ZB:

| Cell Type: | Surface Marker Phenotype: |
|---|---|
| T cells (Total) | L/DNIR780−CD45+CD20−CD3+CD159- |
| B cells | L/DNIR780−CD45+CD20+CD3− |
| NK cells | L/DNIR780−CD45+CD20−CD3−CD159a+ |
| Granulocytes | L/DNIR780−CD45+CD20−CD3−CD159−SSC^{hi}CD11b+ |
| Macrophages | L/DNIR780−CD45+CD20−CD3−CD159−CD163+ |
| HLA-DR+ Myeloid cells | L/DNIR780−CD45+CD20−CD3−CD159−CD163−HLA-DR+ |

TABLE ZF
Definition of Cell Subsets in NHP by Flow Cytometry used in Table ZC

| Cell Type: | Surface Marker Phenotype: |
|---|---|
| B cells | L/DNIR780−CD45+CD20+CD19+ |
| Naive B cells | L/DNIR780−CD45+CD20+CD19+IgM+IgD+CD27−CD21+ |
| Unswitched Memory B cells | L/DNIR780−CD45+CD20+CD19+IgM+IgD+/−CD27+ |
| Switched Memory B cells | L/DNIR780−CD45+CD20+CD19+IgM−IgD−CD27+ |
| Plasmablasts | L/DNIR780−CD45+CD20−CD19+CD38^{hi}CD27− |

### Immunohistochemical Staining and Imaging

Immunohistochemical stained images were collected of cell populations of interest. Briefly, slides were stained using the Leica Bond RX instrument. The reagents used were: Dako Serum free block (catalog #X0909), Cell Signaling CD19 antibody (catalog #90176S), Leica Bond Primary Antibody Diluent (catalog #AR9352), Leica Bond DAB Enhancer (catalog #AR9432), and Biocare Bluing solution (catalog #HTBLU-MX). Polymer, DAB, and hematoxylin were all provided by the Leica Bond Polymer Refine Kit (catalog #DS9800). The Cell Signaling CD19 antibody was used at a dilution of 1:1000 and diluted in Leica Bond Primary antibody diluent. All slides were pretreated using Leica Bond Heat Induced Epitope Retrieval 2 for 20 minutes. Slides were blocked using Dako S block for 5 minutes, treated with primary antibody Cell Signaling CD19 for 60 minutes at room temperature, followed by a Polymer treatment for 16 minutes at room temperature. Both DAB and Hematoxylin staining occurred over 10 minutes at room temperature. Slides were incubated in Leica Bond DAB Enhancer for 5 minutes at room temp. Bluing solution was left on for 1 minute at room temp. Slides were mounted with Polyscience Poly-mount (catalog #24176-120) and coverslipped. Slides were scanned using a Hamamatsu Nanozoomer S60 whole slide scanner.

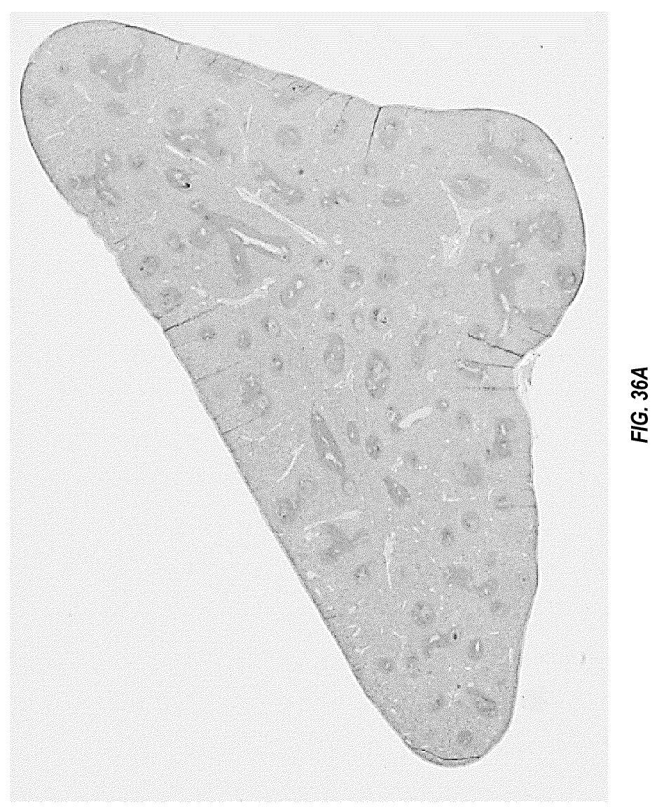

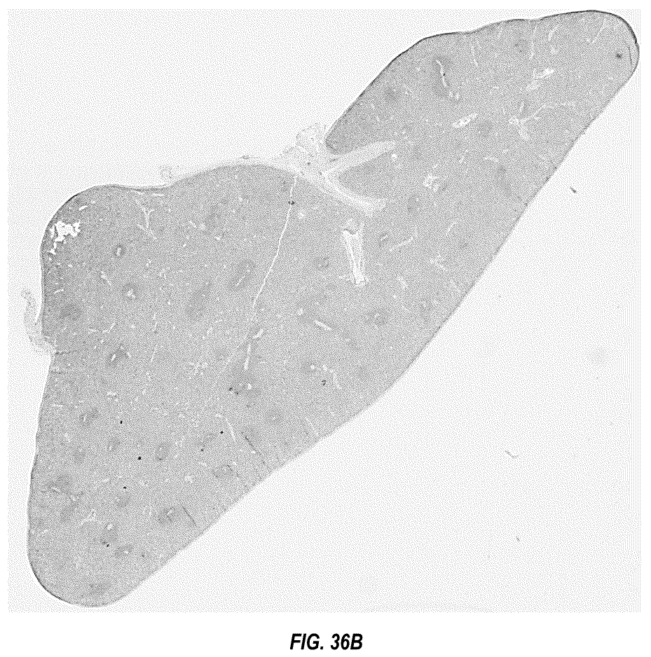

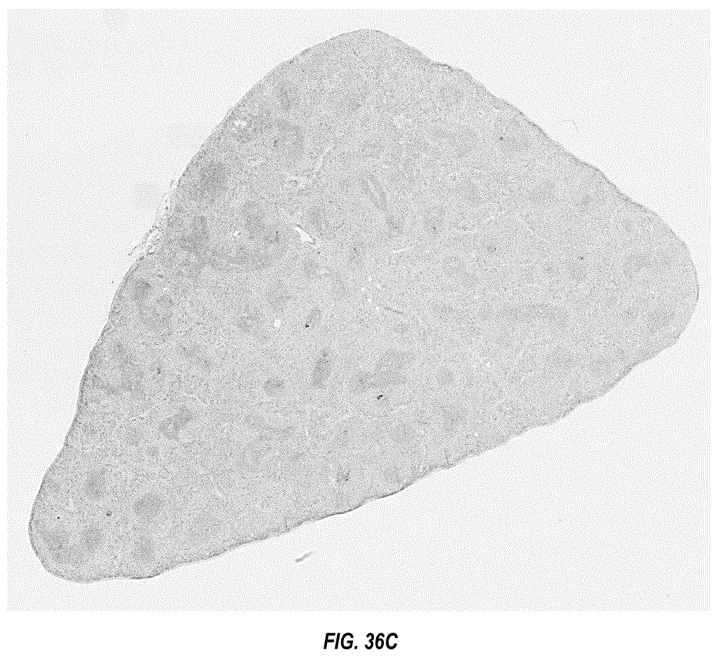

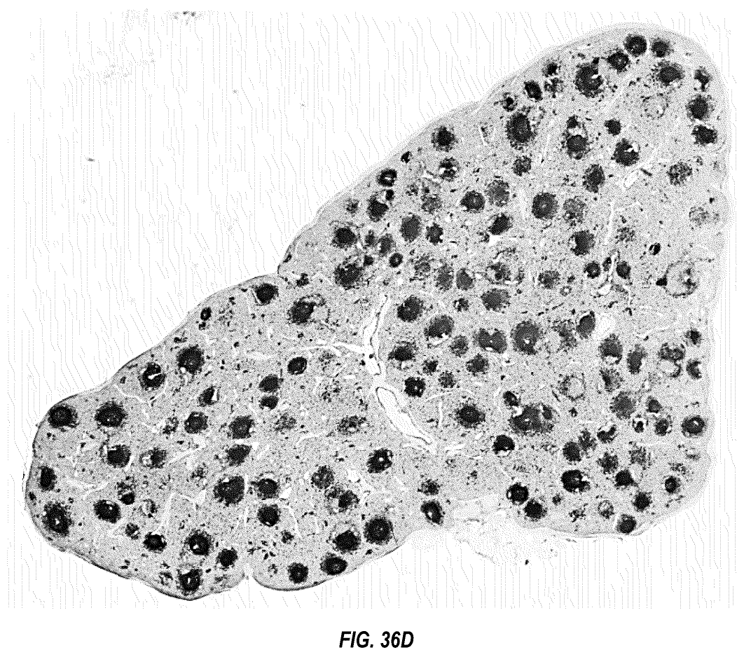

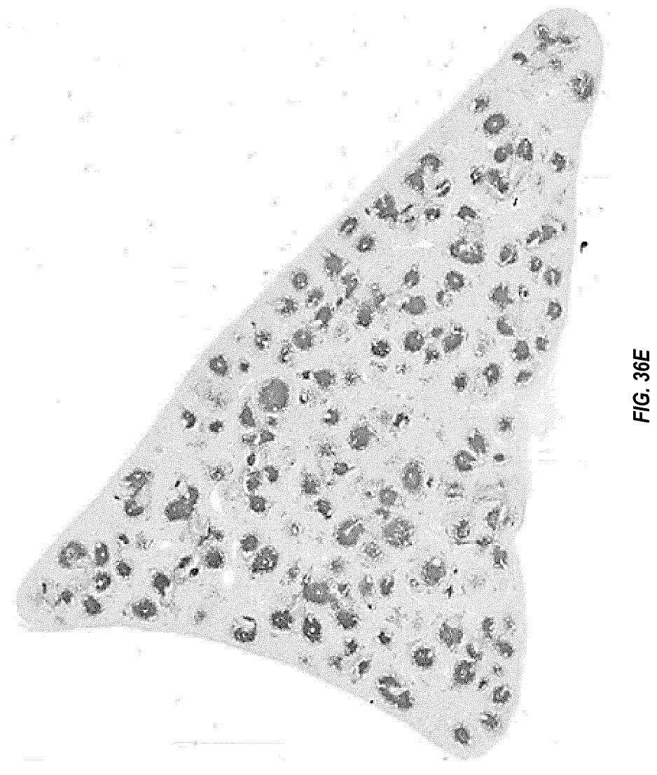

Results are shown in FIGS. 36A-36E. Brown (darker) spots indicate positive CD19 staining while blue (lighter) spots are nuclei counterstains, providing contrast. A reduction in CD19 positive staining in the B-cell follicles, as well as the red pulp of the spleen, was observed in all three groups treated with F-S2 (FIGS. 36A-36C), as compared to the PBS (FIG. 36D) or F-S1 (FIG. 36E) control groups.

### Example 16: LNP oCAR Dose-Dependent B Cell Depletion in NSG-SGM3 Humanized Mice

LNP formulations were prepared as described in Example 2B. Immunodeficient NOD SCID gamma (NSG) mice with transgenic expression of human IL3, GM-CSF (CSF2) and SCF (KITLG) cytokines (NSG-SGM3) were engrafted with human cord blood-derived CD34+ hematopoietic stem cells (hCD34+ NSG-SGM3), 20 weeks old, were pre-bled at Day −6 and on Days 1 and 4 administered (1) 1×PBS (control), (2) formulation F-S1, containing oRNA expressing the VHH reporter, at 1.0 mg/kg, 0.3 mg/kg, 0.1 mg/kg, 0.03 mg/kg, 0.01 mg/kg, or 0.003 mg/kg (3) formulation F-S2, containing the oRNA expressing the CD19 CAR (oCAR), at 1.0 mg/kg, 0.3 mg/kg, 0.1 mg/kg, 0.03 mg/kg, 0.01 mg/kg, or 0.003 mg/kg. At 24 hours following administration on Day 1 (first dose), reporter or CAR expression was measured, and B cell depletion was measured in the blood. Blood was collected into EDTA tubes via submandibular bleed and 75 μL was plated into a 2-mL deep well plate (USA Scientific 1896-2000). Lysis of red blood cells was performed with 2 mL of 1×BD Pharm Lyse Buffer (BD Biosciences 555889) according to manufacturer's protocol prior to staining for flow cytometry. At 72 hours following administration on Day 4 (second dose), reporter or CAR expression was measured by flow cytometry, and B cell depletion was measured in the blood, spleen, and bone marrow by flow cytometry. Blood was collected and processed as described above. Whole spleens were manually dissociated into single cell suspension by gently pressing the spleen through a 70 μm filter with a plunger and washed with autoMACS Running Buffer (Miltenyi Biotec 130-091-221). Red blood cells were lysed using ACK Lysing Buffer (Thermo Fisher A1049201) and washed twice with autoMACS Running Buffer prior to passing through a 40 μm filter. Splenocytes were counted (Cellaca MX, PerkinElmer MX-AOPI), diluted, plated (1,000,000 per well) in a 96-well round bottom plate (Corning 378), and stained for flow cytometry. Bone marrow was flushed from femurs using centrifugation, and red blood cells were lysed with ACK Lysing buffer, as described above. Entire bone marrow sample was plated in a 96-well round bottom plate and stained for flow cytometry. The staining panel disclosed in Table ZG and the cell population definitions in Table ZH were utilized in the above procedure.

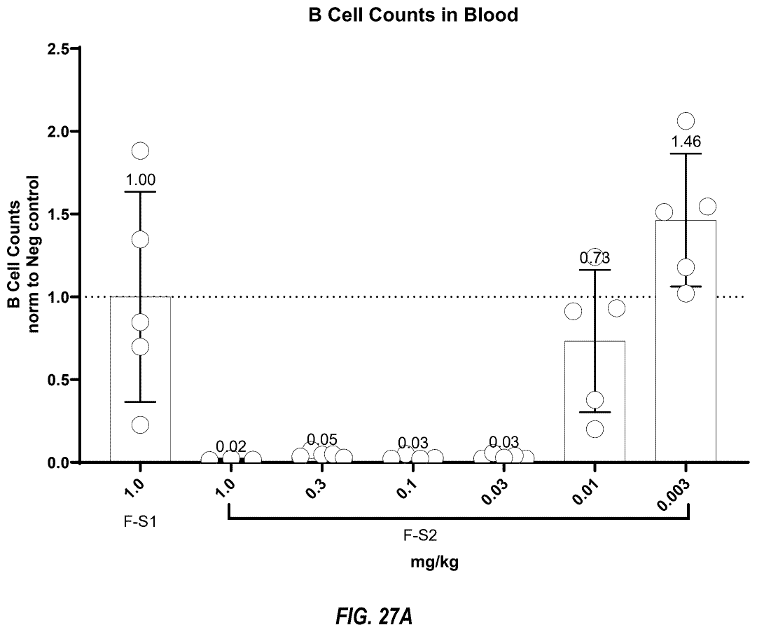

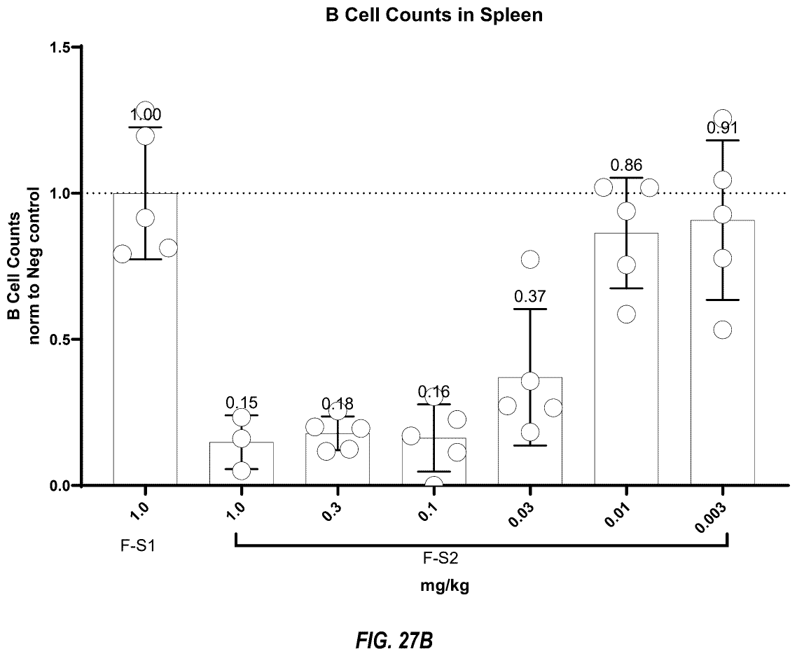

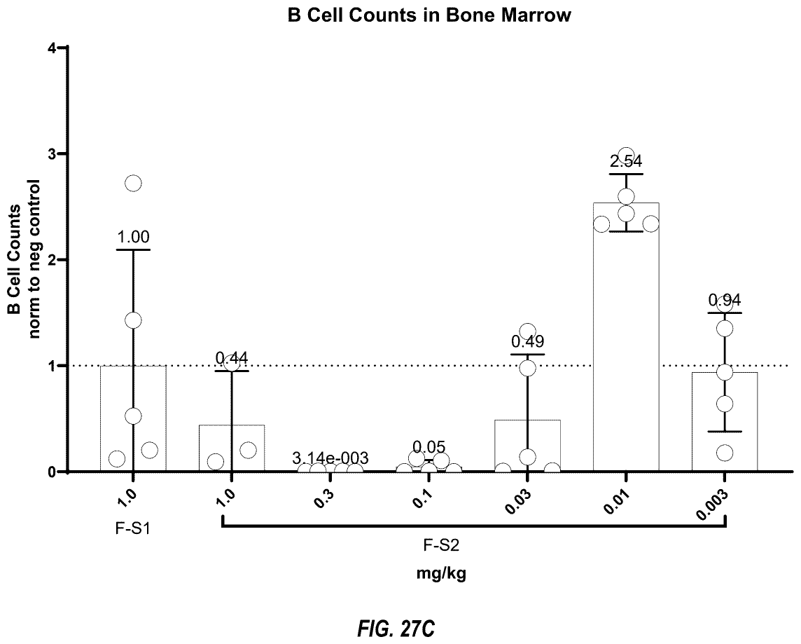

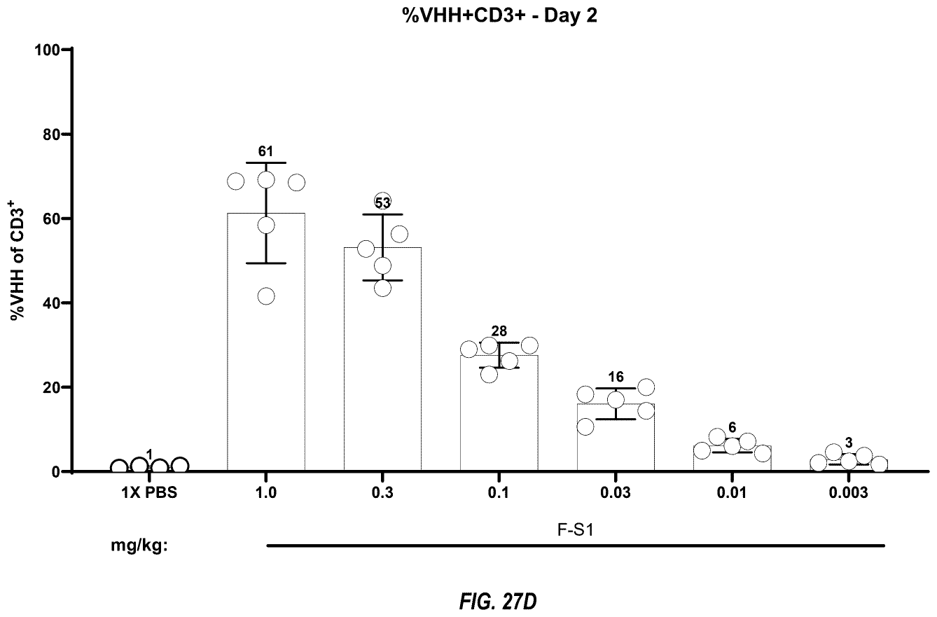

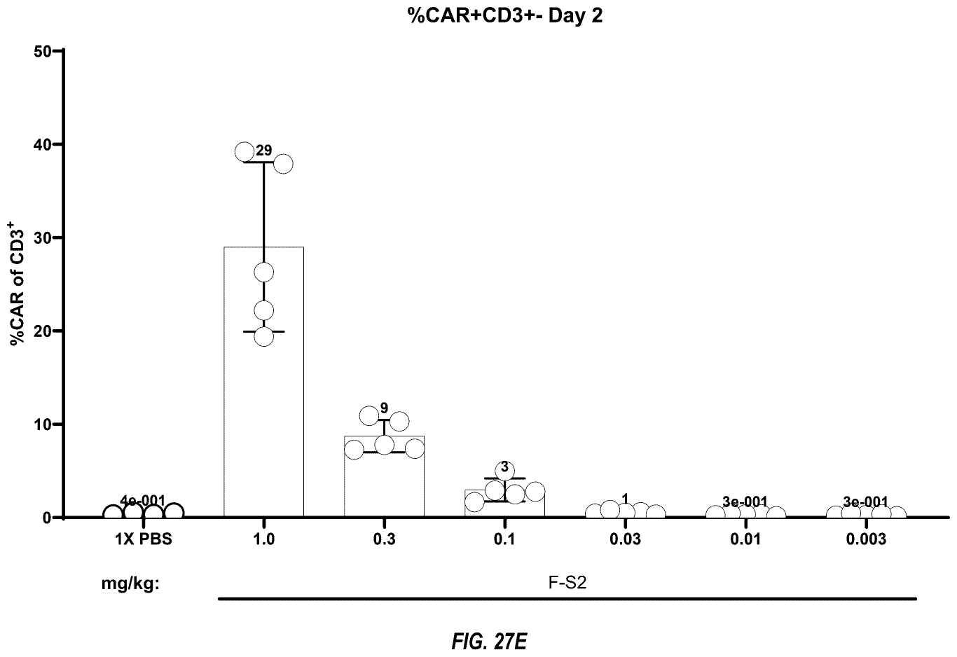

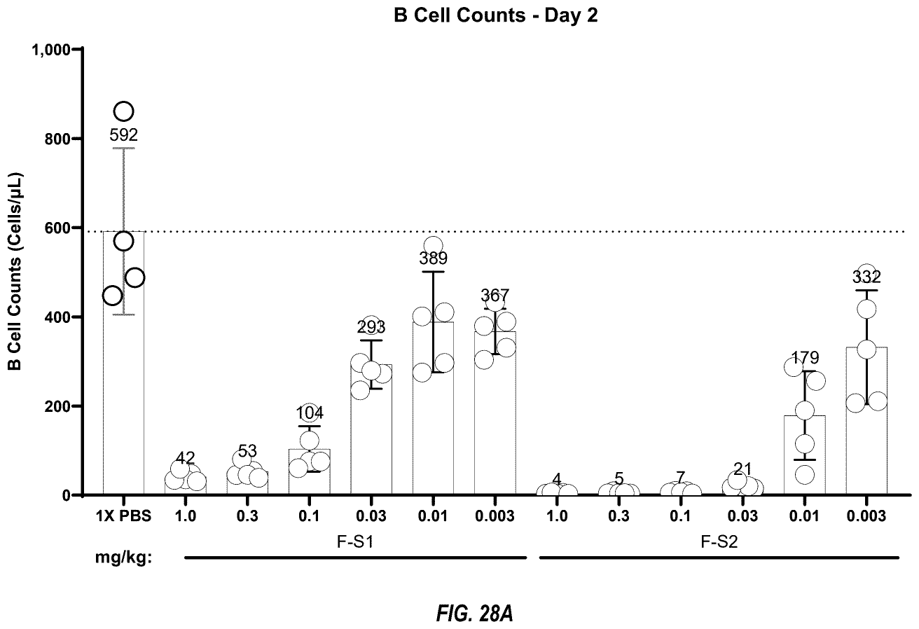

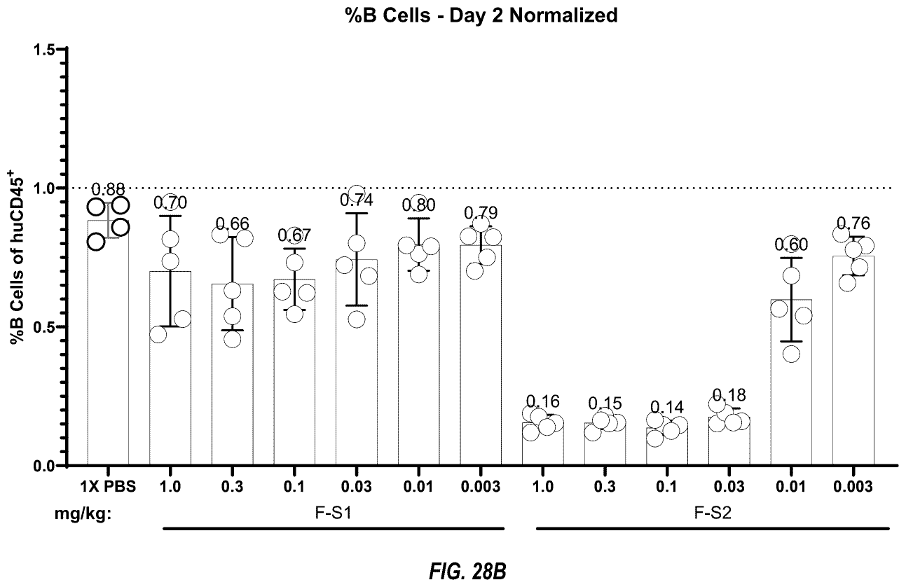

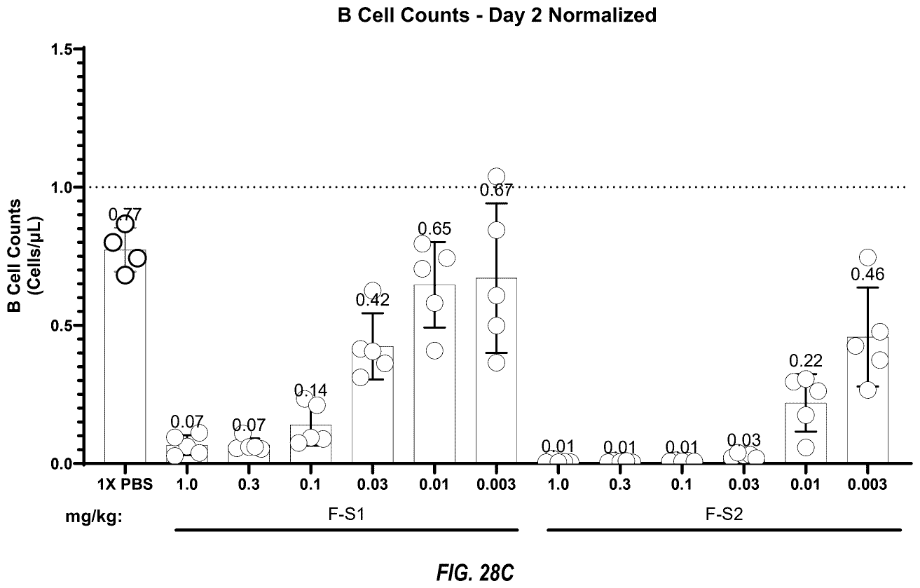

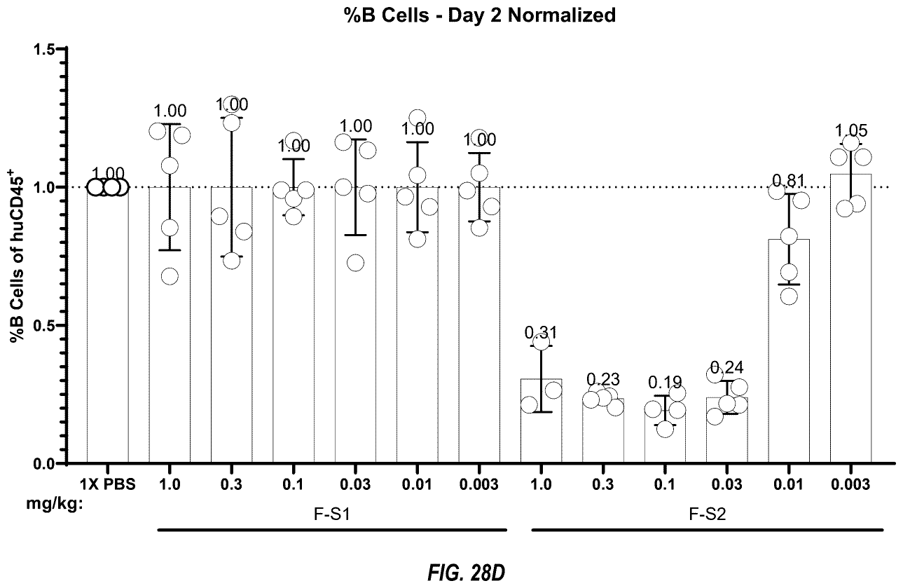

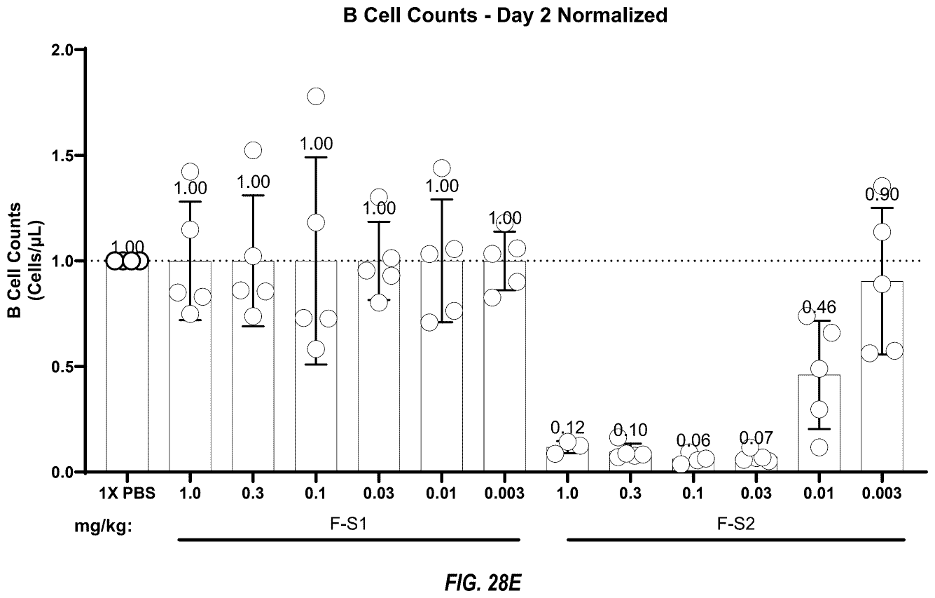

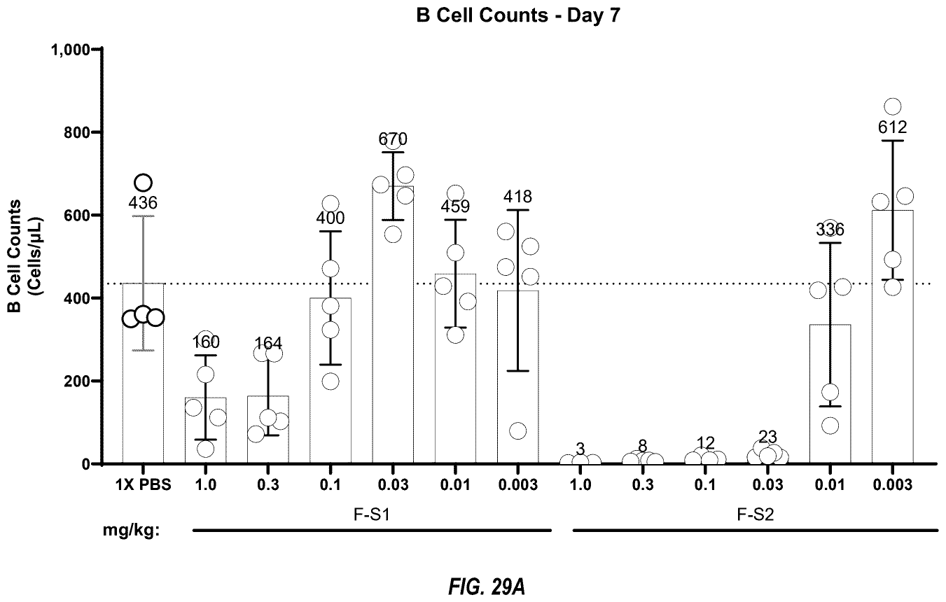

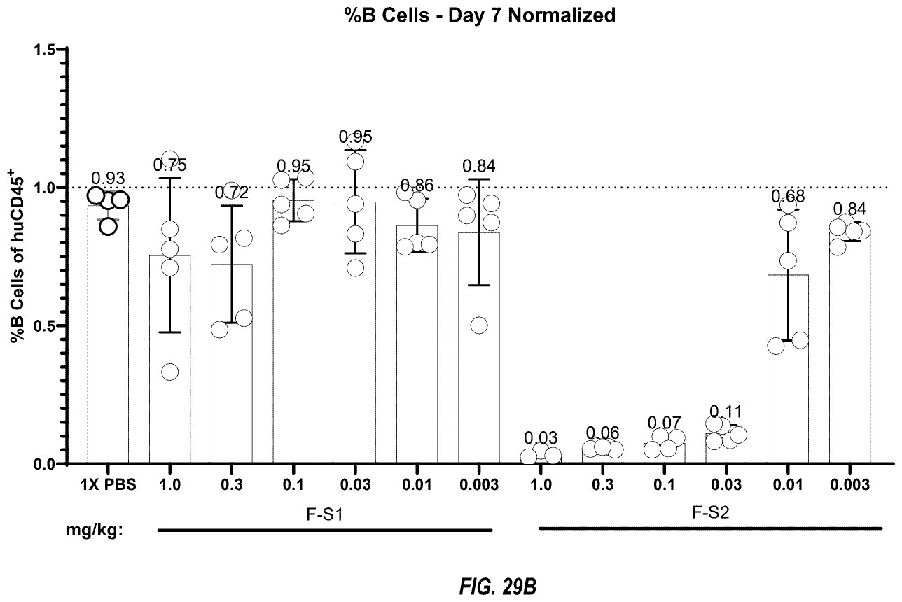

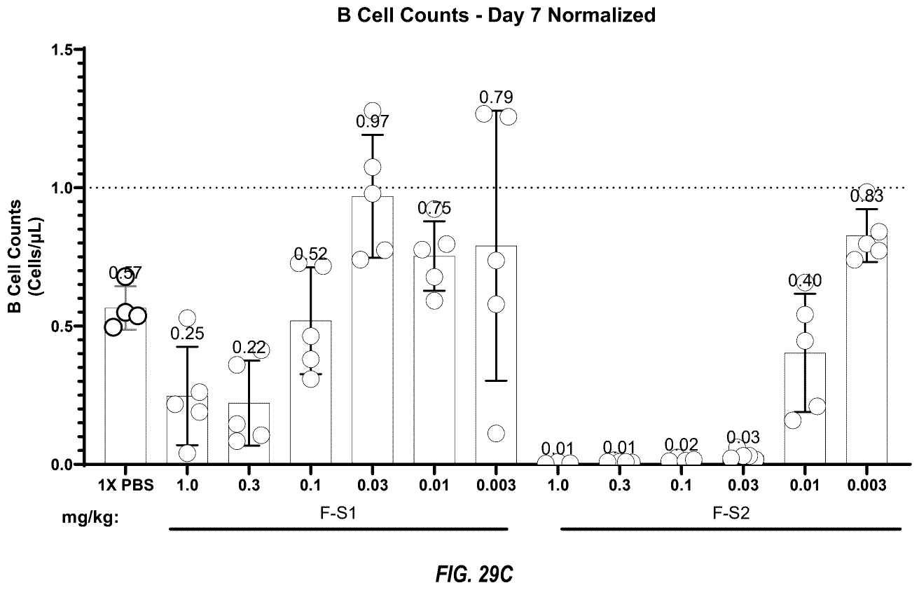

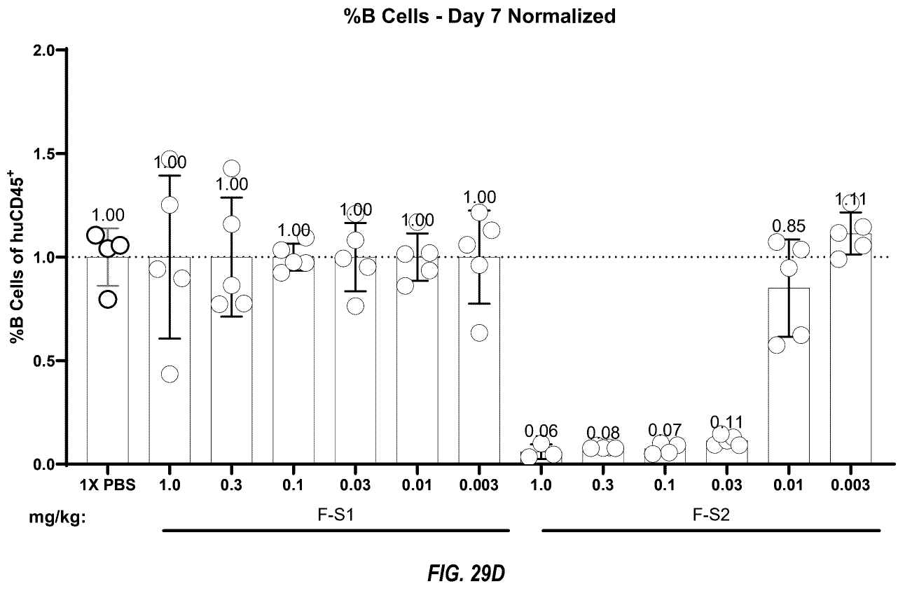

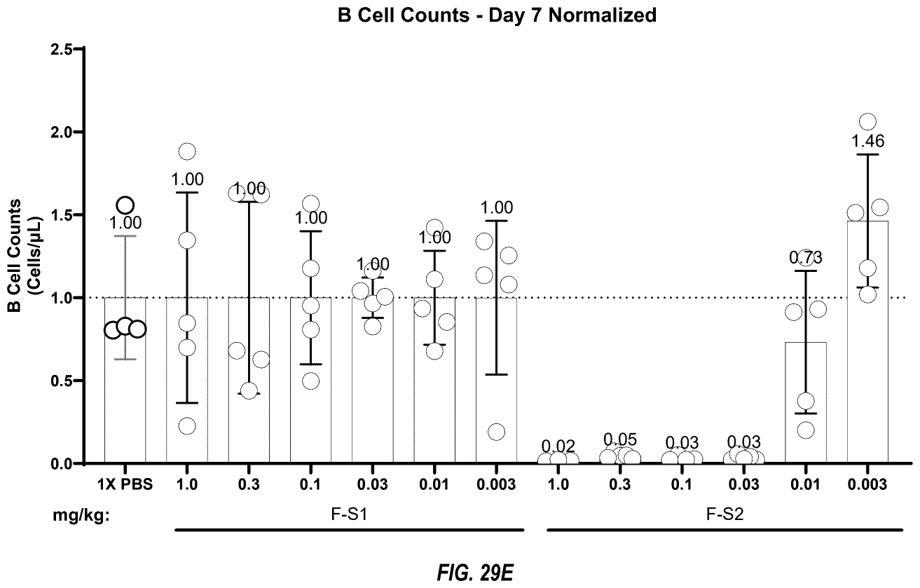

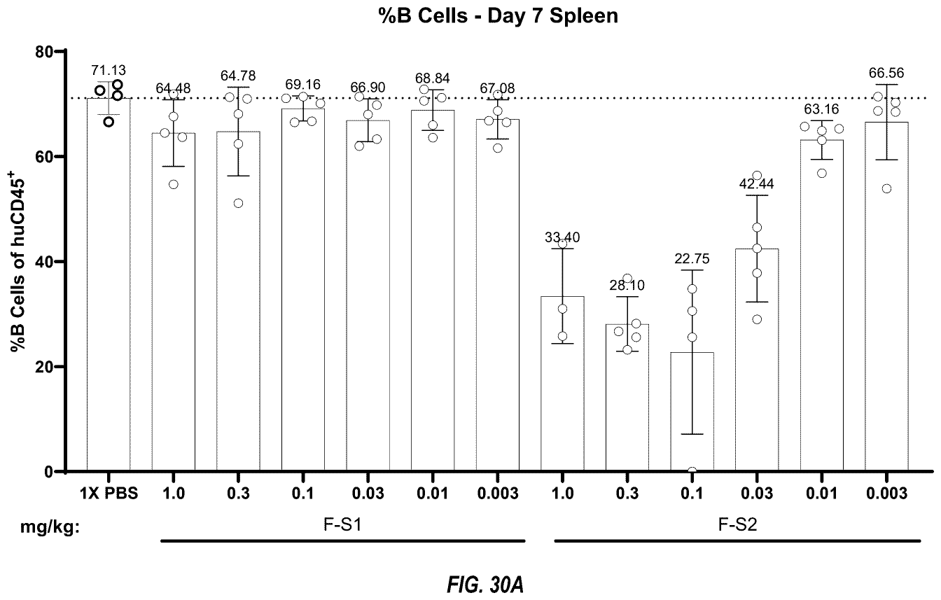

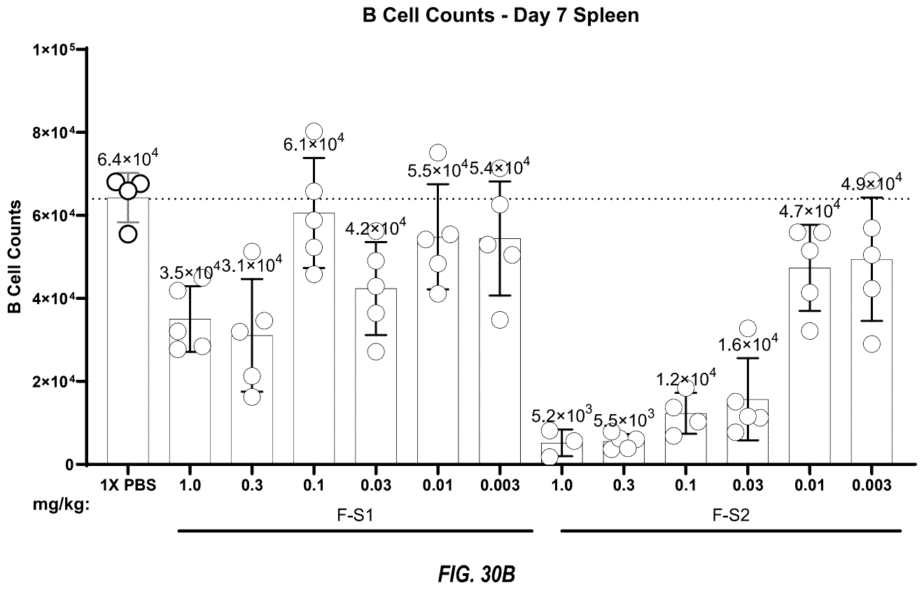

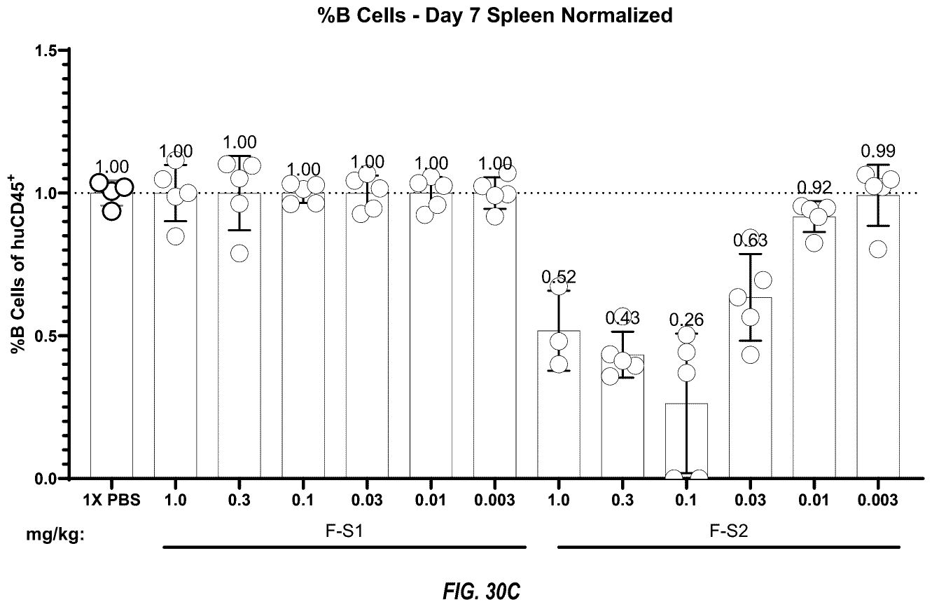

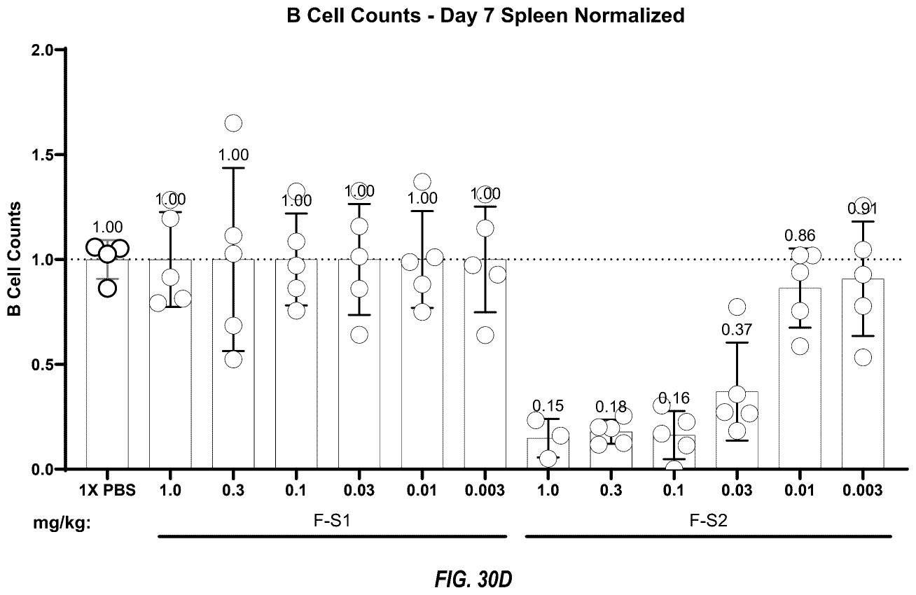

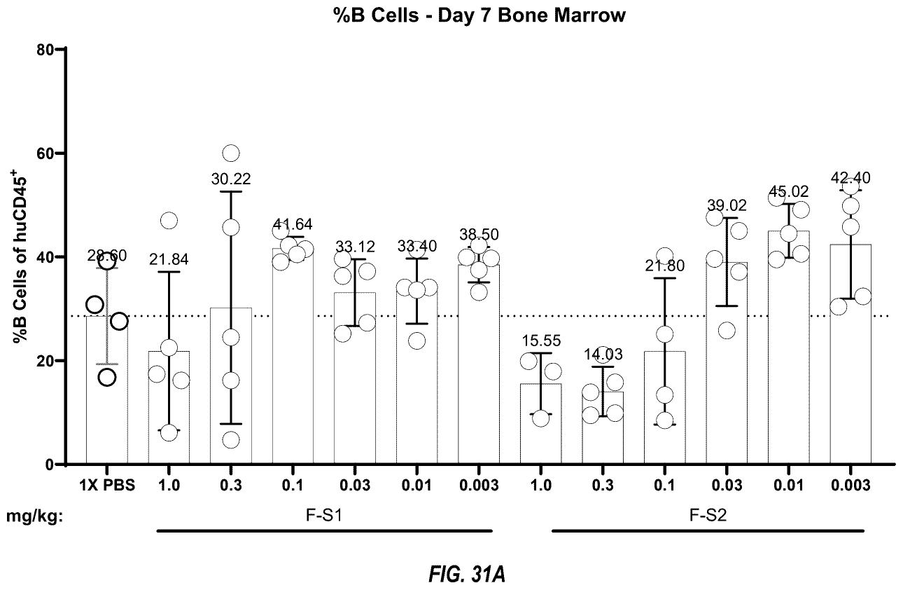

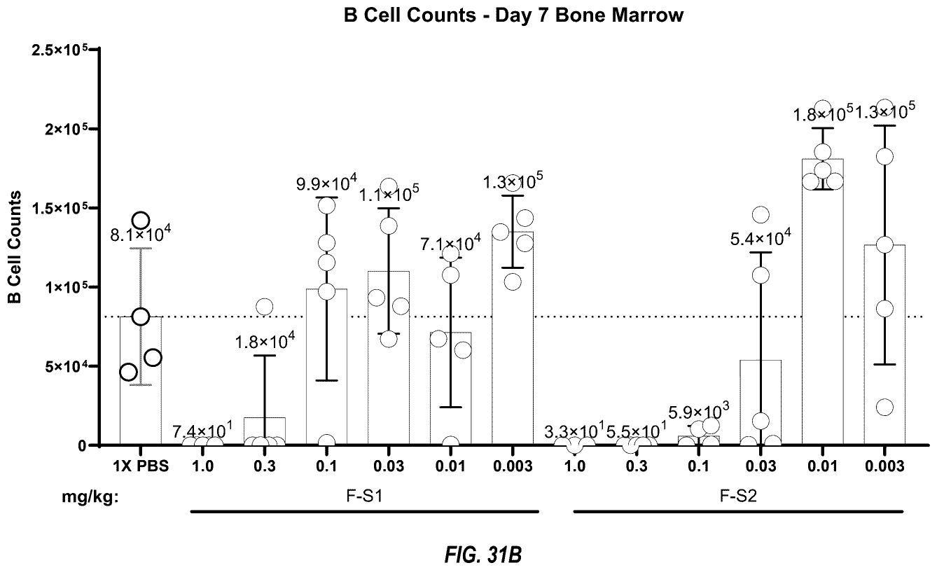

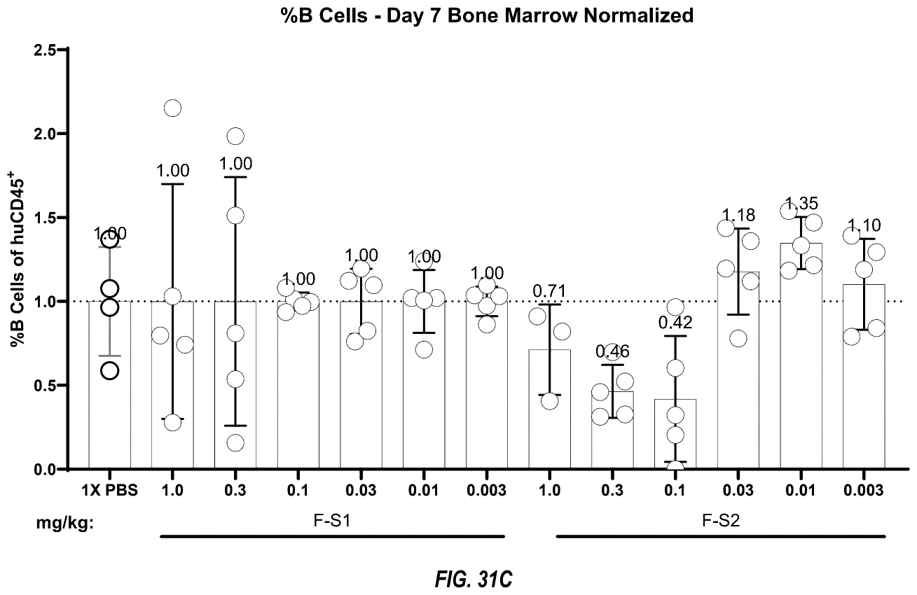

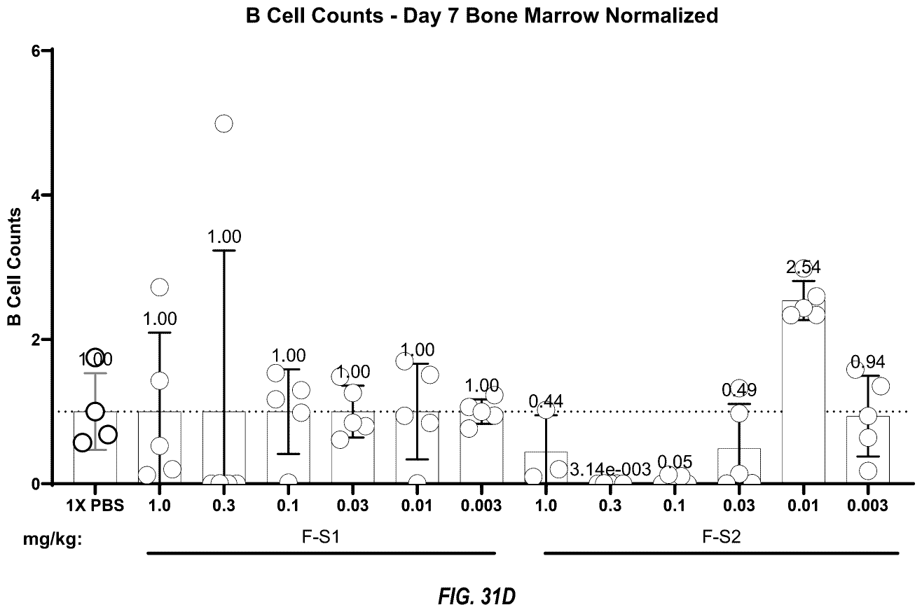

Results are reported in FIGS. 27A through 31D. F-S1 demonstrated dose-dependent reporter (VHH) expression and delivery to peripheral blood lymphocytes from samples collected 24 hours following administration on Day 1 (first dose) (FIG. 27D), ie. samples collected on Day 2. F-S2 demonstrated dose-dependent CD19 CAR expression and delivery to peripheral blood 24 hours following administration on Day 1 (first dose) (FIG. 27E), ie. samples collected on Day 2. F-S2 demonstrated dose-dependent peripheral blood B cell depletion 24 hours following administration on Day 1 (first dose, FIGS. 28A-28E), ie. samples collected on Day 2, and 72 hours following administration of the second dose on Day 4, ie. on Day 7 (second dose, FIGS. 29A-29E). F-S2 demonstrated a dose-dependent B cell depletion in the spleen (FIGS. 30A-30D) and bone marrow (FIGS. 31A-31D) 72 hours following administration of the second on Day 4, ie. on Day 7 (second dose).

TABLE ZG
Panel for CD19CAR and Reporter Expression and B Cell Depletion

| Staining | | | | | | |
|---|---|---|---|---|---|---|
| Step | Target | Fluorophore | Clone | Manufacturer | Catalog | Dilution |
| Live/Dead | Viability | Near IR-780 | N/A | Invitrogen | Number | 1000 |
| Surface | Anti-mouse | AF700 | 30-F11 | Biolegend | 103128 | 200 |
| | CD45 | | | | | |
| | Anti-human | BV421 | 2H7 | Biolegend | 302330 | 200 |
| | CD20 | | | | | |
| | Anti-human | BV650 | UCHT1 | Biolegend | 300468 | 200 |
| | CD3 | | | | | |
| | Anti-human | BV785 | HI30 | Biolegend | 304048 | 200 |
| | CD45 | | | | | |
| | Anti-human | PE-Cy7 | HIB19 | Biolegend | 302216 | 200 |
| | CD19 | | | | | |
| Reporter | CD19CAR | Biotin | rCD19- | Miltenyi | 130-129- | 50 |
| | | | biotin | Biotec | 550 | |
| Primary | VHH | iFluor 488 | 96A3F5 | GenScript | A01862 | 100 |
| Reporter | Streptavidin | PE | N/A | Biolegend | 405204 | 100 |
| Secondary | | | | | | |

TABLE ZH
Definition of Cell Subsets in SGM3 Humanized Mice by Flow Cytometry

| Cell Type: | Surface Marker Phenotype: |
|---|---|
| Human Lymphocytes | L/D NIR780⁻muCD45⁻huCD45⁺ |
| B Cells | L/D NIR780⁻muCD45⁻huCD45⁺CD20⁺CD19⁺ |
| T Cells | L/D NIR780⁻muCD45⁻huCD45⁺CD20⁻CD19⁻CD3⁺ |
| VHH-Expressing- | L/D NIR780⁻muCD45⁻huCD45⁺VHH+ |
| Human Lymphocytes | |
| CD19CAR-Expressing- | L/D NIR780⁻muCD45⁻huCD45⁺CD19CAR+ |
| Human Lymphocytes | |
| VHH-Expressing-T Cells | L/D NIR780⁻muCD45⁻huCD45⁺CD20⁻CD19⁻CD3⁺VHH⁺ |
| CD19CAR-Expressing- | L/D NIR780⁻muCD45⁻huCD45⁺CD20⁻CD19⁻ |
| T Cells | CD3⁺CD19CAR⁺ |

### Example 17: LNP oCAR Multi-Dose Potency in NSG-SGM3 Humanized Mice

LNP formulations were prepared as described in Example 2B3. Immunodeficient NOD SCID gamma (NSG) mice with transgenic expression of human IL3, GM-CSF (CSF2) and SCF (KITLG) cytokines (NSG-SGM3) were engrafted with human cord blood-derived CD34+ hematopoietic stem cells (hCD34+ NSG-SGM3), 16 weeks old, were pre-bled at Day −7 and on Days 1 and 4 administered (1) 1×PBS (control), (2) formulation F-S1, containing oRNA expressing the VHH reporter, at 0.1 mg/kg & 0.03 mg/kg (3) formulation F-S2, containing the oRNA expressing the CD19 CAR (oCAR), at 0.1 mg/kg & 0.03 mg/kg for 2 doses (2×Q3D). Cohorts receiving a single dose were pre-bled at Day −7 and on Day 4 administered (1) 1×PBS (control), (2) formulation F-S1, containing oRNA expressing the VHH reporter, at 0.1 mg/kg & 0.03 mg/kg (3) formulation F-S2, containing the oRNA expressing the CD19 CAR (oCAR), at 0.1 mg/kg & 0.03 mg/kg. At 24 hours following administration of the first dose, reporter or CAR expression was measured, and B cell depletion was measured in the blood. Blood was collected into EDTA tubes via submandibular bleed and 50-70 μL was plated into a 2-mL deep well plate (USA Scientific 1896-2000). Lysis of red blood cells was performed with 2 mL of 1×BD Pharm Lyse Buffer (BD Biosciences 555889) according to manufacturer's protocol prior to staining for flow cytometry. At 72 hours following the final administration, reporter or CAR expression was measured by flow cytometry, and B cell depletion was measured in the blood, spleen, and bone marrow by flow cytometry. Blood was collected and processed as described above. Whole spleens were manually dissociated into single cell suspension by gently pressing the spleen through a 70 μm filter with a plunger and washed with autoMACS Running Buffer (Miltenyi Biotec 130-091-221). Red blood cells were lysed using ACK Lysing Buffer (Thermo Fisher A1049201) and washed twice with autoMACS Running Buffer prior to passing through a 40 μm filter. Splenocytes were counted (Cellaca MX, PerkinElmer MX-AOPI), diluted, plated (1,000,000 per well) in a 96-well round bottom plate (Corning 378), and stained for flow cytometry. Bone marrow was flushed from femurs using centrifugation, and red blood cells were lysed with ACK Lysing buffer, as described above. Entire bone marrow sample was plated in a 96-well round bottom plate and stained for flow cytometry. The staining panel disclosed in Table ZG-II and the cell population definitions in Table ZH (Example 16) were utilized in the above procedure.

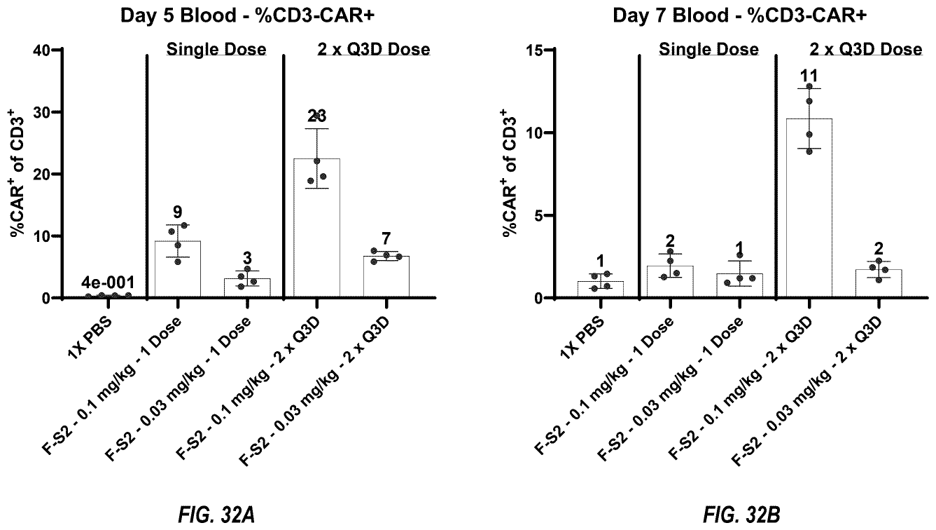

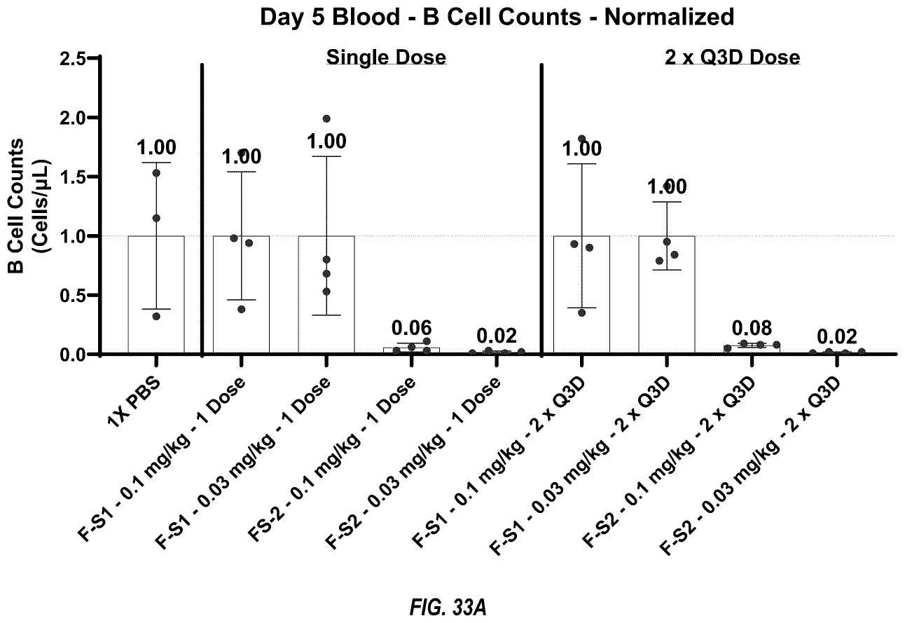

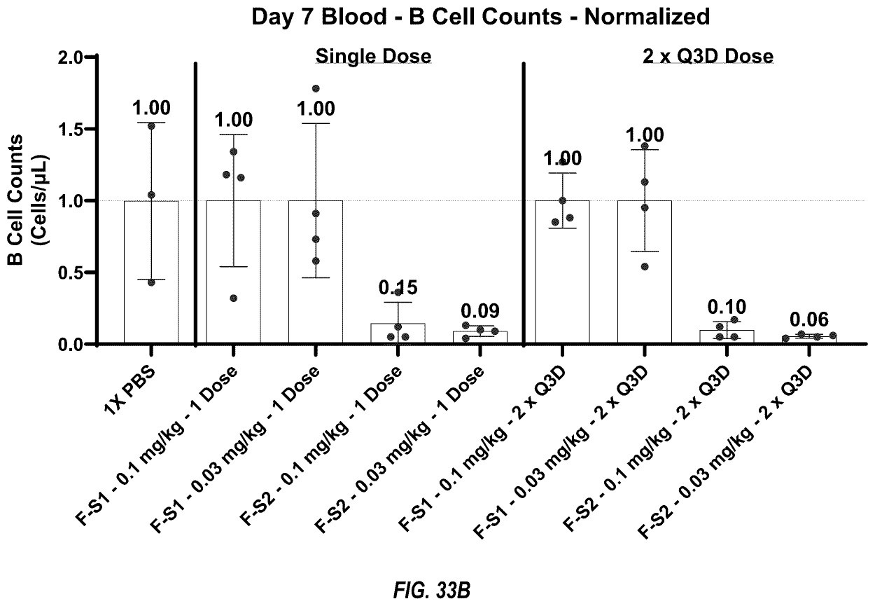

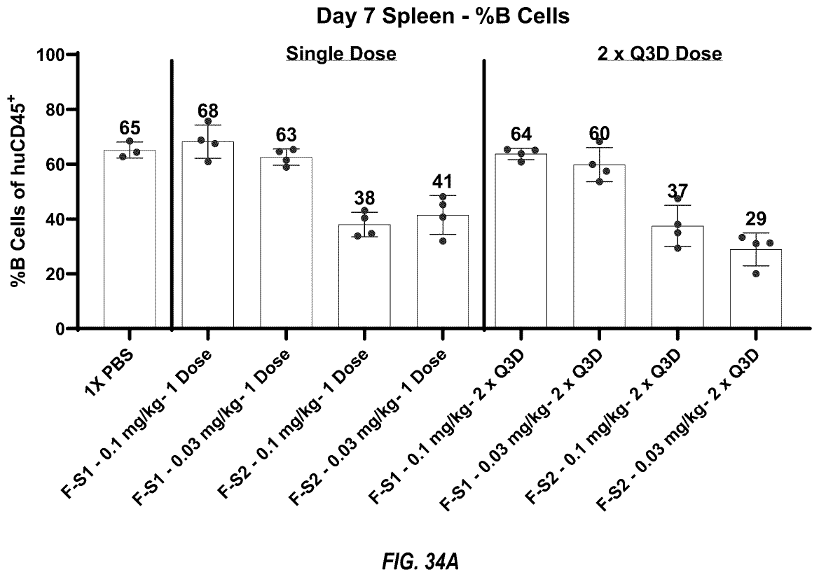

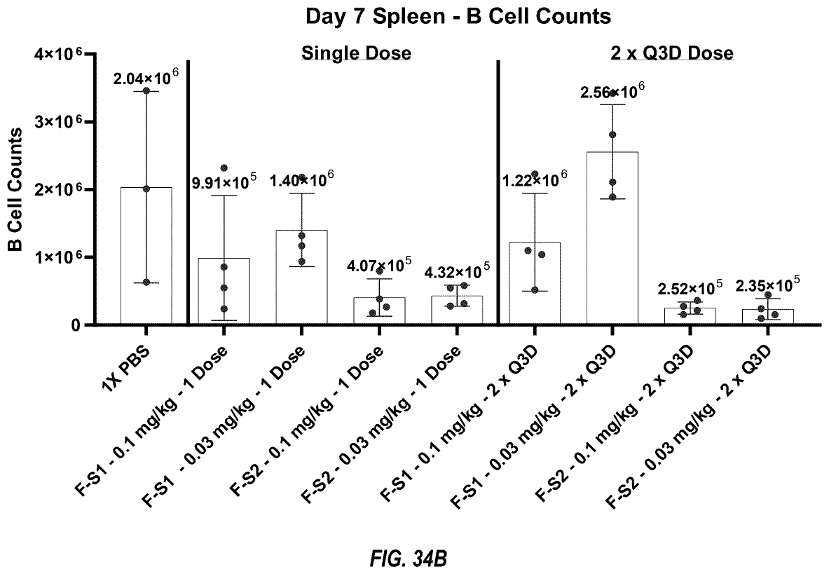

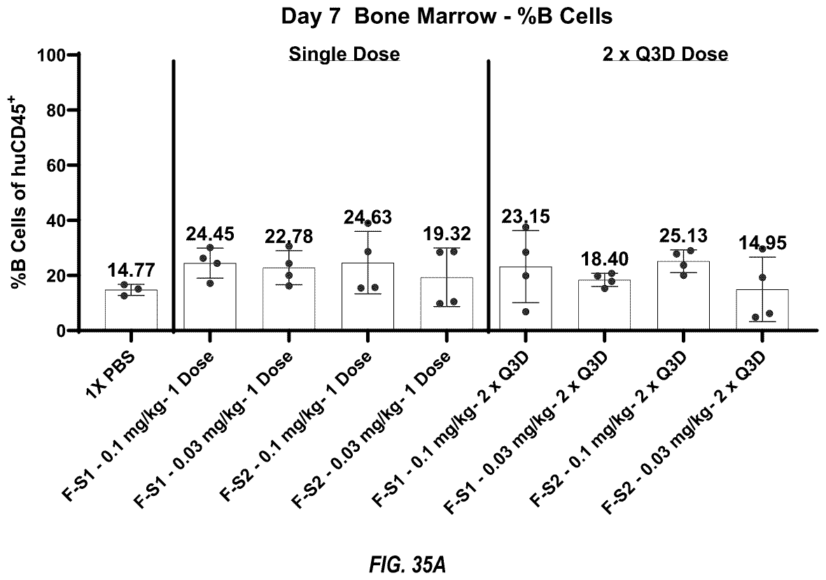

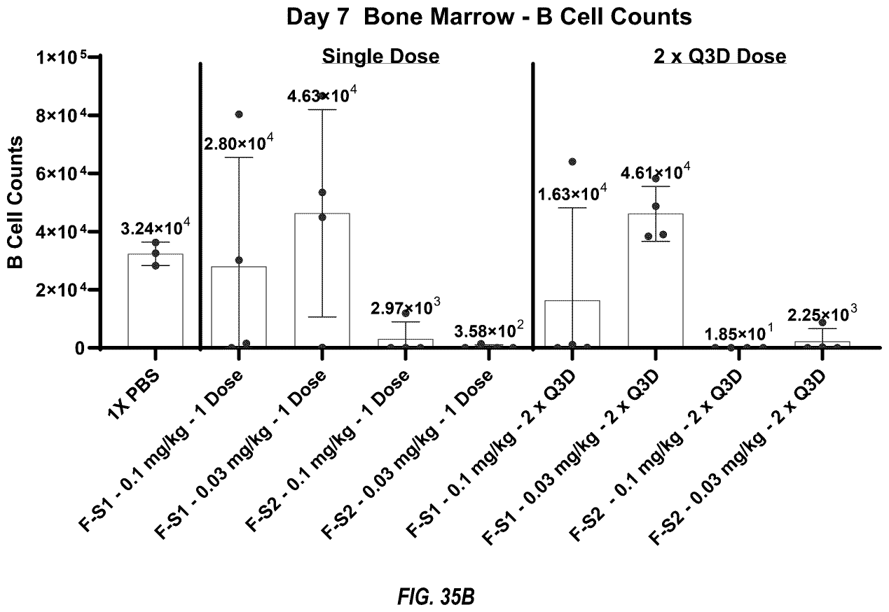

Results are reported in FIGS. 32A through 35B. F-S1 demonstrated dose-dependent reporter (VHH) expression and delivery to peripheral blood lymphocytes 24 hours following administration of the last dose. VHH expression increased upon the administration of a second dose in mice compared to the single dose cohorts. F-S2 demonstrated dose-dependent CD19 CAR expression and delivery to peripheral blood 24 hours following administration of the last dose (FIGS. 32A-32B). CD19 CAR expression increased upon administration of a second dose in mice compared to the single dose cohorts. F-S2 demonstrated dose-dependent peripheral blood B cell depletion 24 hours and 72 hours following the last dose (FIGS. 33A-33B). F-S2 demonstrated a dose-dependent B cell depletion in the spleen 72 hours following administration on Day 4 (FIGS. 34A-34B). F-S2 demonstrated bone marrow B cell depletion 72 hours following the last dose (FIGS. 35A-35B). Overall, mice dosed at 2×Q3D demonstrated higher levels of the payload expression and B cell depletion in the blood and spleen compared to the single dose cohorts.

TABLE ZG-II
Panel for CD19CAR and Reporter Expression and B Cell Depletion

| Staining | | | | | Catalog | |
|---|---|---|---|---|---|---|
| Step | Target | Fluorophore | Clone | Manufacturer | Number | Dilution |
| Live/Dead | Viability | Near IR-780 | N/A | Invitrogen | | 1000 |
| Surface | Anti-human | PerCP-Cy5.5 | W18340F | Biolegend | 307810 | 200 |
| | IgD | | | | | |
| | Anti-human | APC | M-T271 | Biolegend | 986904 | 200 |
| | CD27 | | | | | |
| | Anti-mouse | AF700 | 30-F11 | Biolegend | 103128 | 200 |
| | CD45 | | | | | |
| | Anti-human | BV421 | 2H7 | Biolegend | 302330 | 200 |
| | CD20 | | | | | |
| | Anti-human | BV650 | UCHT1 | Biolegend | 300468 | 200 |
| | CD3 | | | | | |
| | Anti-human | BV785 | HI30 | Biolegend | 304048 | 200 |
| | CD45 | | | | | |
| | Anti-human | PE-Cy7 | HIB19 | Biolegend | 302216 | 200 |
| | CD19 | | | | | |
| Reporter | CD19CAR | Biotin | rCD19- | Miltenyi Biotec | 130-129- | 50 |
| | | | biotin | | 550 | |
| Primary | VHH | iFluor 488 | 96A3F5 | GenScript | A01862 | 100 |
| Reporter | Streptavidin | PE | N/A | Biolegend | 405204 | 100 |
| Secondary | | | | | | |

### Example 18: LNP Circular oRNA CAR (oCAR) and Linear mRNA CAR (mCAR) in NSG-SGM3 Humanized Mice

LNP formulations were prepared as described in Example 2C. Immunodeficient NOD SCID gamma (NSG) mice with transgenic expression of human IL3, GM-CSF (CSF2) and SCF (KITLG) cytokines (NSG-SGM3) were engrafted with human cord blood-derived CD34+ hematopoietic stem cells (hCD34+ NSG-SGM3), 15 weeks old, were pre-bled at Day −3 and on Days 0 administered (1) 1×PBS (control), (2) F-C1 (IRES sequence Table 4-19 and amino acid binder sequence of Construct UJ, HER2 control), at 0.1 mg/kg, (3) CD19 oCAR F-C2 (IRES sequence of Table 4-19 and amino acid binder sequence of Table 10-50) at 0.1 mg/kg, (4) CD19 oCAR F-C3 (IRES sequence of Table 4-21 and amino acid binder sequence of Table 10-60 and miR122 binding site described herein) at 0.1 mg/kg, (5) CD19 oCAR F-C4 at 0.1 mg/kg, (6) CD19 mCAR F-C5 (5′ cap as described herein and amino acid binder sequence of Table 10-60 and miR122 binding site described herein) at 0.1 mg/kg, and (7) CD19 mCAR F-C6 (5′ cap as described herein and same binder as in F-C4) at 0.1 mg/kg. For all of the mRNA constructs described in this example, N1-methyl-pseudouridine was used for all U. All of the CARs described in this example comprised CD8 signal, CD28 hinge, CD28 transmembrane, CD28 signal, and CD3zeta domains (28ζ). At 24 hours following administration of the dose, CAR expression was measured, and B cell depletion was measured in the blood. Blood was collected into EDTA tubes via submandibular bleed and 50-70 μL was plated into a 2-mL deep well plate (USA Scientific 1896-2000). Lysis of red blood cells was performed with 2 mL of 1×BD Pharm Lyse Buffer (BD Biosciences 555889) according to manufacturer's protocol prior to staining for flow cytometry. At 192 hours following the final administration, CAR expression was measured by flow cytometry, and B cell depletion was measured in the blood & spleen by flow cytometry. Blood was collected and processed as described above. Whole spleens were manually dissociated into single cell suspension by gently pressing the spleen through a 70 μm filter with a plunger and washed with autoMACS Running Buffer (Miltenyi Biotec 130-091-221). Red blood cells were lysed using ACK Lysing Buffer (Thermo Fisher A1049201) and washed twice with autoMACS Running Buffer prior to passing through a 40 μm filter. Splenocytes were counted (Cellaca MX, PerkinElmer MX-AOPI), diluted, plated (1,000,000 per well) in a 96-well round bottom plate (Corning 378), and stained for flow cytometry. Bone marrow was flushed from femurs using centrifugation, and red blood cells were lysed with ACK Lysing buffer, as described above. Entire bone marrow sample was plated in a 96-well round bottom plate and stained for flow cytometry. The staining panel disclosed in Table ZJ.

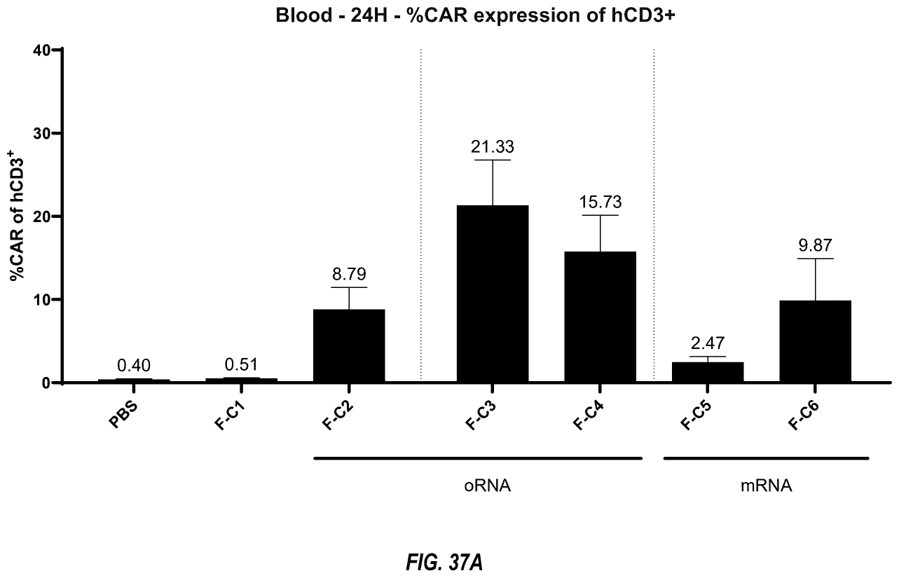

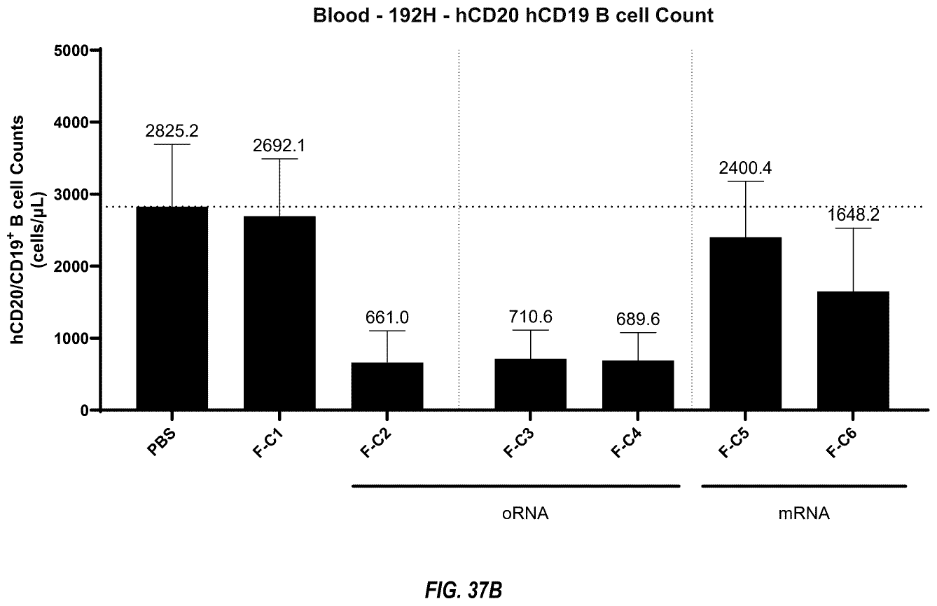

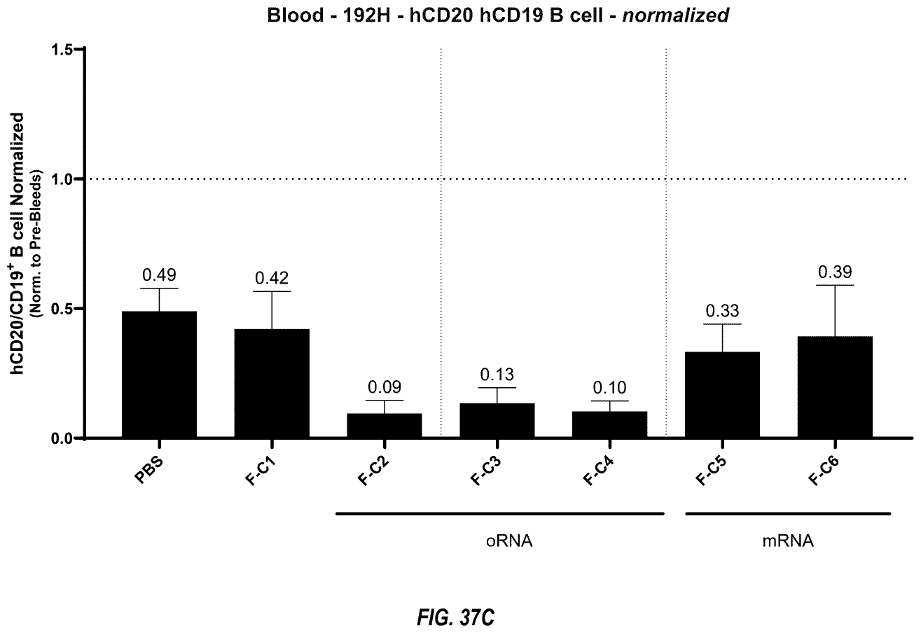

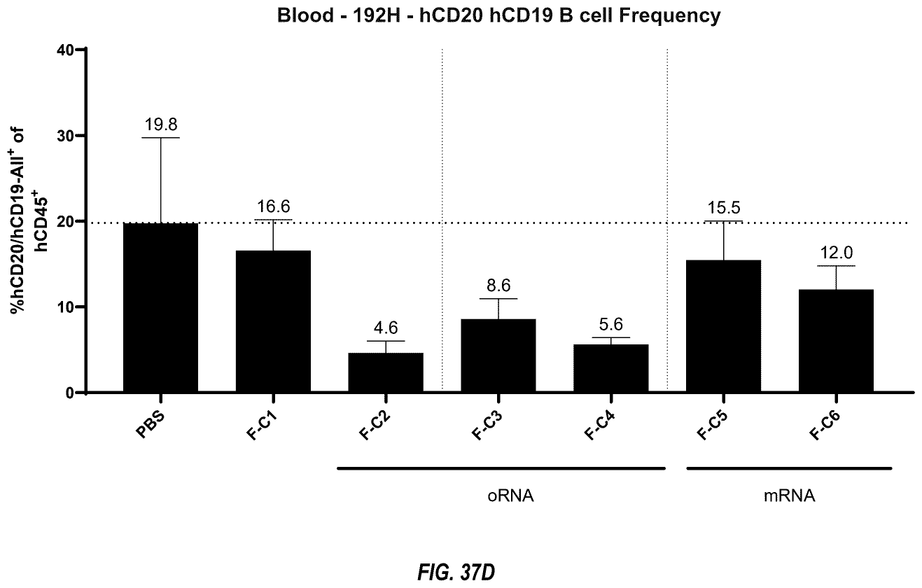

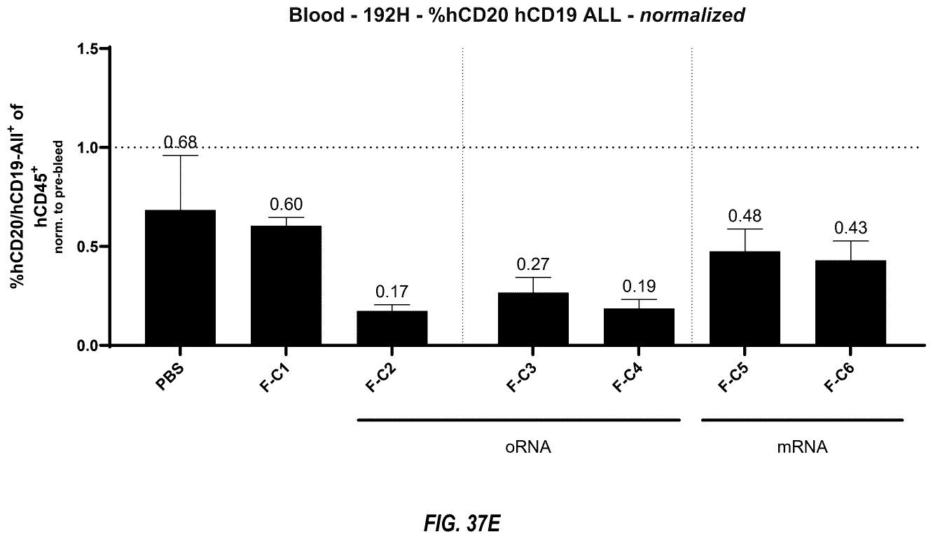

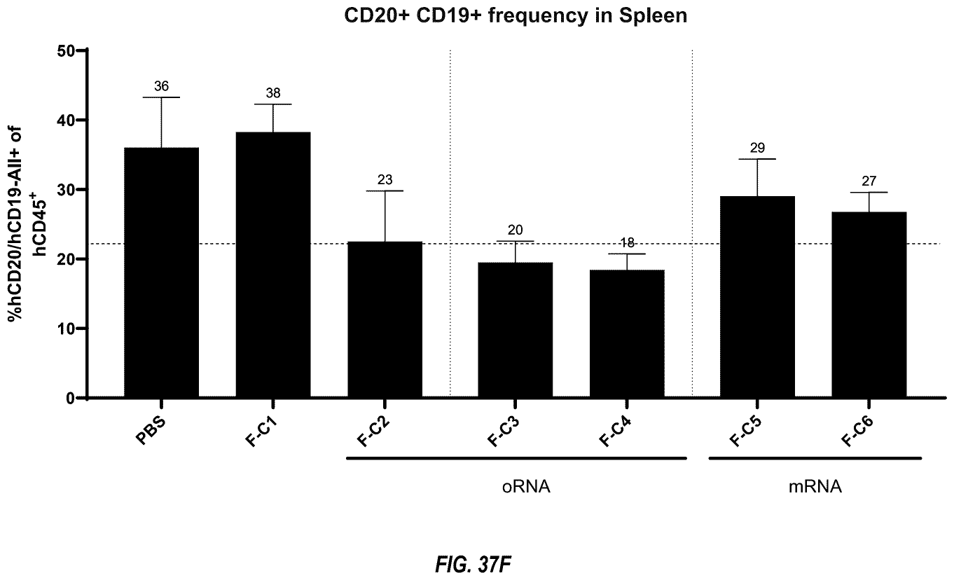

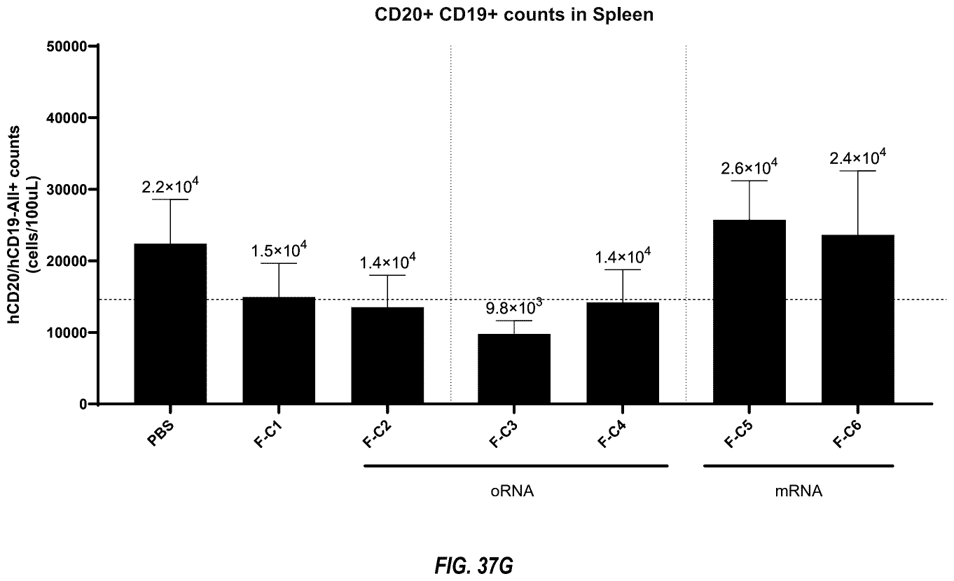

Results are reported in FIGS. 37A through 37G. As shown in FIG. 37A, F-C2 through F-C6 all demonstrated CAR expression and delivery to peripheral blood lymphocytes 24 hours following administration of the last dose. As shown in FIGS. 37B through 37E, F-C2 through F-C6 all demonstrated peripheral blood B cell depletion 192 hours following the last dose, with oRNA F-C2, F-C3, and F-C4 demonstrating superior depletion as compared to mRNA formulations (F-C5 and F-C6).

TABLE ZJ
Panel for CD19CAR and Reporter Expression and B Cell Depletion

| Staining | | | | | Catalog | |
|---|---|---|---|---|---|---|
| Step | Target | Fluorophore | Clone | Manufacturer | Number | Dilution |
| Live/Dead | Viability | Near IR-780 | N/A | Invitrogen | L34994 | 1000 |
| Surface | Anti-human | PerCP-Cy5.5 | W18340F | Biolegend | 307810 | 200 |
| | IgD | | | | | |
| | Human | AF647 | N/A | R&D Systems | AFR1129 | 200 |
| | ErbB2/Her2 | | | | | |
| | Fc His | | | | | |
| | Anti-mouse | AF700 | 30-F11 | Biolegend | 103128 | 200 |
| | CD45 | | | | | |
| | Anti-human | BV421 | 2H7 | Biolegend | 302330 | 200 |
| | CD20 | | | | | |
| | Anti-human | BV650 | UCHT1 | Biolegend | 300468 | 200 |
| | CD3 | | | | | |
| | Anti-human | BV785 | HI30 | Biolegend | 304048 | 200 |
| | CD45 | | | | | |
| | Anti-human | PE-Cy7 | HIB 19 | BD | 560728 | 200 |
| | CD19 | | | | | |
| Reporter | CD19CAR | Biotin | rCD19- | Miltenyi | 130-129- | 50 |
| Primary | | | biotin | Biotec | 550 | |
| Reporter | Streptavidin | PE | N/A | Biolegend | 405204 | 100 |
| Secondary | | | | | | |

### Example 19: LNP oCAR and mCAR In Vitro Human T Cells

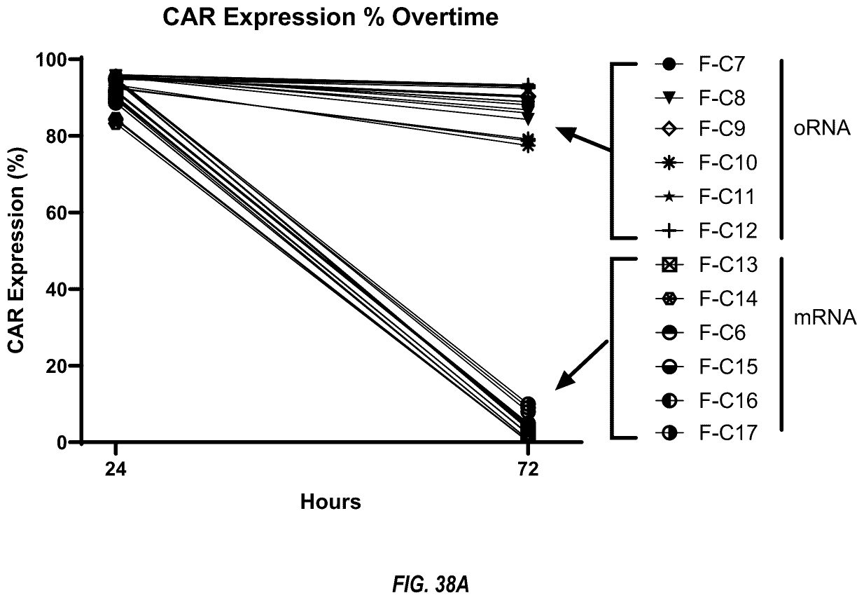

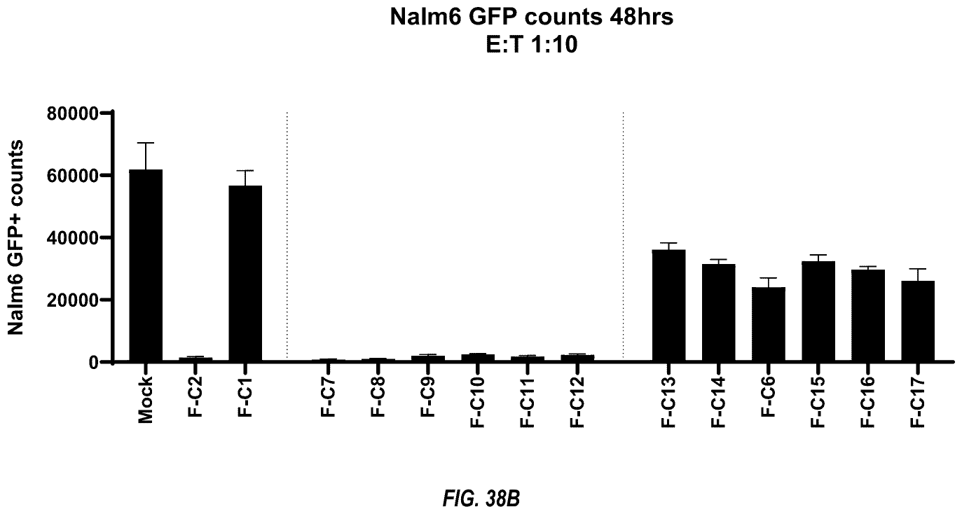

Frozen human T cells were thawed and activated with Stemcell CD3/CD28 antibody cocktail for 96 hours. Post activation T cells were transfected with LNP containing either linear mRNA CD19.28ζ CAR (F-C6 (described above), F-C13 (5′ cap as described herein and amino acid binder sequence of Table 10-53) and miR122 binding site described herein), F-C14 (5′ cap as described herein and amino acid binder sequence of Table 10-60 and miR122 binding site described herein), F-C15 (5′ cap as described herein and amino acid binder sequence of Table 10-54 and miR122 binding site described herein), F-C16 (5′ cap as described herein and amino acid binder sequence of Table 10-58 and miR122 binding site described herein), F-C17 (5′ cap as described herein and amino acid binder sequence of Table 10-59 and miR122 binding site described herein)) or circular RNA CD19.28ζ CAR (F-C7 (IRES sequence of Table 4-21 and amino acid binder sequence of Table 10-53 and miR122 binding site described herein), F-C8 (IRES sequence of Table 4-21 and amino acid binder sequence of Table 10-60 (alternative codon optimization compared to F-C3)), F-C9 (same binder as in F-C4 and F-C9 further comprising miR122 binding site described herein), F-C10 (IRES sequence of Table 4-21 and amino acid binder sequence of Table 10-54 and miR122 binding site described herein), F-C11 (IRES sequence of Table 4-21 and amino acid binder sequence of Table 10-58 and miR122 binding site described herein), F-C12 (IRES sequence of Table 4-21 and amino acid binder sequence of Table 10-59 and miR122 binding site described herein)) as shown above at 0.1 ng of RNA-LNP cargo per 1×10⁵ cells. For all of the mRNA constructs described in this example, N1-methyl-pseudouridine was used for all U. T cells that were transfected but had no circular RNA (mock), F-C2, and F-C1 served as controls. After LNP transfection, cells were allowed to recover for 24 hours, following which they were assessed for expression by FACS at 24 and 72 hours post transfection. CD19 CAR was detected using soluble recombinant CD19 fluorokines (soluble proteins conjugated to fluorophores) (FIG. 38A). Transfected T cells were co-cultured with Nalm6 lymphoblastic leukemia cells at a 1:10 ratio of effector to target cells for 48 hours. Target cell killing was evaluated by FACS, where oCAR demonstrated superior CAR-mediated cytotoxicity of target cells compared to the mCAR-treated cohorts (FIG. 38B).

### Example 20: mRNA Synthesis

### Materials

DNA template. The DNA template comprised a T7 promoter with AGG as initiator sequence, a 5′ untranslated region (5′ UTR), an open reading frame (ORF), a 3′ untranslated region (3′ UTR), a polyA tail and a BspQI linearization site.

In vitro transcription. The IVT reaction system contained Tris (pH 7.5, Sigma-Aldrich), magnesium chloride (Sigma-Aldrich), spermidine (Sigma-Aldrich), dithiothreitol (DTT, Thermo Fisher), adenosine triphosphate (ATP, Hongene), cytidine triphosphate (CTP, Hongne), guanosine triphosphate (GTP, Hongene), N1-methylpseudouridine triphosphate (m1ψ, Hongene), Cleancap m6 (Trilink), RNase inhibitor (Hongene), pyrophosphatase inorganic (yeast, Hongene), T7 RNA polymerase (Hongene) and linearized DNA template (in-house). After the reaction, DNA template was digested by DNase I (Hongene) and then quenched by ethylenediaminetetraacetic acid (EDTA, Thermo Fisher).

mRNA purification. Affinity purification was performed via ÄKTA avant™ chromatography system (Cytiva) with a monolithic oligo dT (18) column (Sartorius). Buffers contained sodium chloride (NaCl, Thermo Fisher), Tris (pH 7.4, Thermo Fisher) and EDTA (pH 8.0, Thermo Fisher) were used during the purification. Tangential flow filtration (TFF, Repligen) was performed with 100 kDa cutoff MidiKros hollow fiber filter. Sodium citrate (pH 6.5, Thermo Fisher) was used as the storage buffer for purified mRNA.

### Methods

DNA template preparation. A plasmid comprising the DNA template was cloned and transformed into 5-alpha competent E. coli (NEB). The bacteria were grown at 37° C. overnight and the plasmid was then extracted via EndoFree Plasmid Giga kit (Qiagen). BspQI (Hongene) was used to linearize the plasmid followed by Amicon Ultra centrifuge filter (100 kDa, Sigma-Aldrich) for dialysis and purification.

In vitro transcription (IVT). The system contained the following: 40 mM Tris, pH 7.5, 16 mM magnesium chloride, 16 mM hydrochloric acid, 2.12 mM spermidine, 10 mM DTT, 5 mM ATP, 5 mM CTP, 5 mM GTP, 5 mM m1ψ, 10 mM Cleancap m6, 0.25 U/μL RNase inhibitor, 0.001 U/μL pyrophosphatase inorganic (yeast), 15 U/μL T7 RNA polymerase and 0.05 g/L linearized DNA template. The reaction was incubated at 37° C. for 2 hours, followed by DNase I treatment (0.05 U/μL) for 20 minutes. 40 mM EDTA was used to quench the reaction.

mRNA purification. A high salt buffer was added to the quenched samples from IVT to make the final concentration of NaCl as 500 mM. The samples were then loaded onto the monolithic oligo dT (18) column, followed by wash with buffer A1 (500 mM NaCl, 10 mM Tris and 1 mM EDTA). A low salt buffer A2 (100 mM NaCl, 10 mM Tris and 1 mM EDTA) was applied to further wash the column. An elution buffer B1 (10 mM Tris and 1 mM EDTA) was then used to elute mRNAs that bind to the column. The eluted samples were applied to TFF to remove the salt and 1 mM sodium citrate, pH 6.5 was used as the dialysis buffer to store the purified mRNAs.

### ENUMERATED EMBODIMENTS

1. An RNA comprising:
a. a translation initiation element comprising a sequence that is at least 80% identical to a sequence selected from Table 4 or Table 5 or a functional fragment thereof, and
b. at least one expression sequence encoding a CD19 binding molecule that is at least 80% identical to a sequence selected from Table 5, Table 9, or Table 10 or a functional fragment thereof.

2. The RNA of embodiment 1, wherein the translation initiation element comprises an internal ribosome entry site (IRES).

3. The RNA of embodiment 1 or 2, wherein the translation initiation element comprises a sequence that is at least 90% identical to the sequence selected from Table 4 or Table 5 or a functional fragment thereof.

4. The RNA of any one of embodiments 1-3, wherein the translation initiation element comprises a sequence that is at least 95% identical to the sequence selected from Table 4 or Table 5 or a functional fragment thereof.

5. The RNA of any one of embodiments 1-4, wherein the RNA is a circular RNA, comprising in the following order:
a. a 3′ self-spliced exon segment;
b. the translation initiation element;
c. the at least one expression sequence encoding a CD19 binding molecule; and
d. a 5′ self-spliced exon segment.

6. The RNA of embodiment 5, wherein the 3′ self-spliced exon segment is a Group I or Group II self-spliced exon segment, and/or the 5′ self-spliced exon segment is a Group I or Group II self-spliced exon segment.

7. The RNA of any one of embodiments 5 or 6, further comprising a 5′ internal duplex and/or a 3′ internal duplex.

8. The RNA of any one of embodiments 5-7, further comprising a 5′ internal spacer and/or a 3′ internal spacer.

9. The RNA of any one of embodiments 5-8, wherein the circular RNA is 50 nucleotides to 15 kilobases in length.

10. A linear RNA comprising at least one expression sequence encoding a CD19 binding molecule that is at least 80% identical to a sequence selected from Table 5, Table 9, or Table 10, or a functional fragment thereof.

11. The RNA of any one of embodiments 1-10, wherein the functional fragment of the CD19 binding molecule comprises a first complementary determining region (CDR1), a second complementary determining region (CDR2), and a third complementary determining region (CDR3) selected from Table 5, Table 9, or Table 10.

12. The RNA of any one of embodiments 1-11, wherein the CD19 binding molecule is a sdAb or a scFv.

13. The RNA of any one of embodiments 1-11, wherein the CD19 binding molecule is a nanobody or a VHH.

14. The RNA of any one of embodiments 1-13, wherein the CD19 binding molecule is a CAR.

15. The RNA of embodiment 14, wherein the CAR comprises a CD28 or 4-1BB costimulatory domain.

16. The RNA of any one of embodiments 1-15, comprising at least one modified A, C, G, or U nucleotide or nucleoside.

17. The RNA of any one of embodiments 1-16, wherein the modified nucleotide or nucleoside is:
a) is selected from one or more of m⁵U (5-methyluridine); m⁶A (N⁶-methyladenosine); s²U (2-thiouridine); Ψ (pseudouridine); Um (2′-O-methyluridine); m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms² hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine(phosphate)); yW (wybutosine); o₂yW (peroxywybutosine); OHyW (hydroxy wybutosine); OHyW* (undermodified hydroxywybutosine); imG (wyosine); m¹mG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G+(archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(β-amino-3-carboxypropyl)uridine); ho⁵U (5-hydroxyuridine); mo⁵U (5-methoxyuridine); cmo⁵U (uridine 5-oxyacetic acid); mCmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine); pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-methylpseudouridine, and N2,N2-dimethyl-6-thio-guanosine; or
b) is selected from one or more of: 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine, 5-(2-amino)propyl uridine, 5-halocytidine, 5-halouridine, 4-acetylcytidine, 1-methyladenosine, 2-methyladenosine, 3-methyicytidine, 6-methyluridine, 2-methylguanosine, 7-methylguanosine, 2,2-dimethylguanosine, 5-methylaminoethyluridine, 5-methyloxyuridine, 7-deaza-adenosine, 6-azouridine, 6-azocytidine, 6-azothymidine, 5-methyl-2-thiouridine, 2-thiouridine, 4-thiouridine, 2-thiocytidine, dihydrouridine, pseudouridine, queuosine, archaeosine, naphthyl substituted naphthyl groups, an O- and N-alkylated purines and pyrimidines, N6-methyladenosine, 5-methylcarbonylmethyluridine, uridine 5-oxyacetic acid, pyridine-4-one, pyridine-2-one, aminophenol, 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, N1-methylpseudouridine, and alkylcarbonylalkylated nucleotides; or
c) is selected from one or more of: 5-methylcytidine, 5-methoxyuridine, 1-methyl-pseudouridine, N6-methyladenosine, pseudouridine, and combinations thereof.

18. A pharmaceutical composition comprising:
a. the RNA of any one of embodiments 1-17; and
b. a lipid nanoparticle comprising:
   i. one or more ionizable lipids;
   ii. one or more phospholipids, in an amount of about 20 mol % to about 60 mol % of the total lipid content of the lipid nanoparticle;
   iii. one or more structural lipids; and
   iv. one or more PEG lipids.

19. The pharmaceutical composition of embodiment 18, wherein the lipid nanoparticle comprises about 25 mol % to about 45 mol % of the one or more ionizable lipids, as a proportion of the total lipid content of the lipid nanoparticle.

20. The pharmaceutical composition of embodiment 18 or 19, wherein the lipid nanoparticle comprises about 15 mol % to about 35 mol % of the one or more structural lipids, as a proportion of the total lipid content of the lipid nanoparticle.

21. The pharmaceutical composition of any one of embodiments 18-20, wherein the lipid nanoparticle comprises about 1 mol % to about 3 mol % of the one or more PEG lipids, as a proportion of the total lipid content of the lipid nanoparticle.

22. The pharmaceutical composition of any one of embodiments 18-21, wherein the lipid nanoparticle comprises (a) about 1 mol % to about 3 mol % of the one or more PEG lipids; (b) about 15 mol % to about 35 mol % of the one or more structural lipids; (c) about 30 mol % to about 60 mol % of the one or more phospholipids; and (d) about 25 mol % to about 45 mol % of the one or more ionizable lipids.

23. The pharmaceutical composition of any one of embodiments 18-22, wherein the lipid nanoparticle comprises (a) about 1.5 mol % to about 2.5 mol % of the one or more PEG lipids; (b) about 20 mol % to about 30 mol % of the one or more structural lipids; (c) about 35 mol % to about 45 mol % of the one or more phospholipids; and (d) about 28 mol % to about 40 mol % of the one or more ionizable lipids.

24. The pharmaceutical composition of any one of embodiments 18-23, wherein the one or more ionizable lipids comprises a compound selected from those depicted in any one of Tables (I-A), (I-B), (I-C), (I-D), (I-E), (I-F), (I-G), (I-H), (I-I), (I-J), (I-K), or (I-L), an enantiomer thereof, or any mixture of enantiomers thereof, or a pharmaceutically acceptable salt of any such compound.

25. The pharmaceutical composition of any one of embodiments 18-23, wherein the one or more ionizable lipids comprises a compound of Formula (AX)

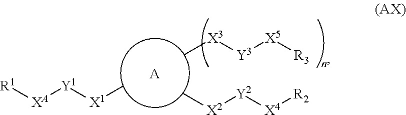

or a pharmaceutically acceptable salt thereof, wherein:
A is selected from an optionally substituted bridged carbocyclic or heterocyclic core selected from the group consisting of:

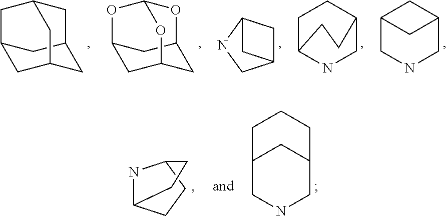

n is and integer selected from 1 or 2;
R¹ is selected from the group consisting of —OH, —OAc, —NR₂,

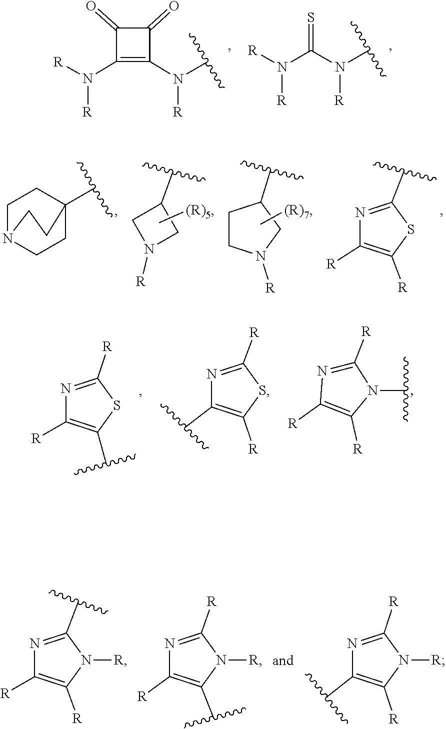

each R is independently —H or C₁-C₆ aliphatic;
X¹ and X^{A} are each independently a bond or optionally substituted C₁-C₆ aliphatic;
Y¹ is selected from the group consisting of

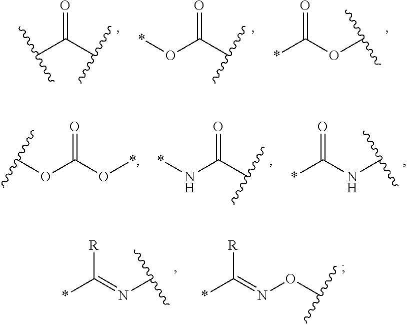

and bond; wherein the bond marked with an “*” is attached to X¹;
each X² and X³ is independently a bond or optionally substituted C₁-C₁₂ aliphatic;
each Y² and Y³ is independently selected from the group consisting of

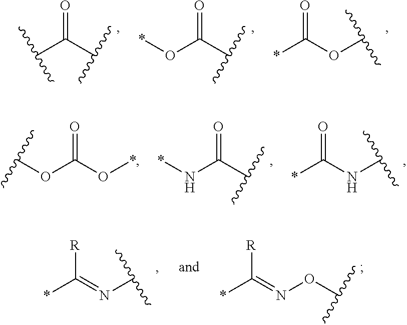

wherein the bond marked with an “*” is attached to X² or X³;
each X⁴ and X⁵ is independently optionally substituted C₁-C₆ aliphatic;
R² is —CH(OR⁶)(OR⁷), —CH(SR⁶)(SR⁷), —CH(R⁶)(R⁷), —R¹⁰, optionally substituted C₅-C₁₈ aliphatic, or optionally substituted C₁-C₁₄ aliphatic-R¹⁰, wherein one or more methylene linkages of R² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R³ is independently —CH(OR⁸)(OR⁹), —CH(SR⁸)(SR⁹), —CH(R⁸)(R⁹), —R¹¹, optionally substituted C₅-C₁₈ aliphatic, or optionally substituted C₁-C₁₄ aliphatic-R¹¹, wherein one or more methylene linkages of R³ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁶ and R⁷ are each independently optionally substituted —C₁-C₁₄ aliphatic, —R¹⁰, or optionally substituted —C₁-C₁₄ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁸ and R⁹ are each independently optionally substituted —C₁-C₁₄ aliphatic, —R¹¹, or optionally substituted —C₁-C₁₄ aliphatic-R¹¹; wherein one or more methylene linkages of R⁸ and R⁹ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R¹⁰ and R¹¹ is independently an optionally substituted cylic, bicyclic, bridged bicyclic, multicyclic or bridged multicyclic C₄-C₁₄ cycloalkyl or optionally substituted cylic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

26. The pharmaceutical composition of any one of embodiments 18-25, wherein the one or more phospholipids are selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin, or combinations of 2, 3, 4, 5, 6, 7, 8, 9, 10, 11, or 12 of the above phospholipids.

27. The pharmaceutical composition of any one of embodiments 18-25, wherein the one or more phospholipids are selected from DSPC and egg sphingomyelin, or a combination thereof.

28. The pharmaceutical composition of any one of embodiments 18-27, wherein the one or more structural lipids are selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.

29. The pharmaceutical composition of any one of embodiments 18-28, wherein the one or more PEG lipids is selected from DMG-PEG2k, DSPE-PEG2k, DSG-PEG2k, DMPE-PEG2k, DPPE-PEG2k and mixtures thereof.

30. The pharmaceutical composition of any one of embodiments 18-25, 28 and 29, wherein the one or more phospholipids comprises one or more selected from phosphatidylcholine, phosphatidylserine, phosphoethanolamine, and sphingoid lipids or a combination thereof.

31. The pharmaceutical composition of any one of embodiments 18-25, 28 and 29, wherein the one or more phospholipids comprises 1,2-distearoyl-sn-glycero-3-phosphocholine (DSPC), sphingomyelin or a combination thereof.

32. The pharmaceutical composition of any one of embodiments 18-25, 28, 29, 30 and 31, wherein the one or more phospholipids comprises two or more phospholipids, such that no single phospholipid makes up more than 30 mol % of the total lipid content of the nanoparticle.

33. The pharmaceutical composition of any one of embodiments 18-25, 28 and 29, wherein the lipid nanoparticle comprises about 40 mol % 1,2-distearoyl-sn-glycero-3-phosphocholine (DSPC).

34. The pharmaceutical composition of any one of embodiments 18-25, 28 and 29, wherein the lipid nanoparticle comprises about 40 mol % sphingomyelin.

35. The pharmaceutical composition of any one of embodiments 18-29, wherein the phospholipid is sphingomyelin, the structural lipid is cholesterol, and the PEG lipid is DMG-PEG2k.

36. The pharmaceutical composition of any one of embodiments 18-29, wherein the ionizable lipid is a compound of Formula (AX), the phospholipid is sphingomyelin, the structural lipid is cholesterol, and the PEG lipid is DMG-PEG2k.

37. The pharmaceutical composition of any one of embodiments 18-29, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of the one or more ionizable lipids.

38. The pharmaceutical composition of any one of embodiments 18-29, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of an ionizable lipid of Formula (AX).

39. The pharmaceutical composition of any one of embodiments 18-29, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of the one or more ionizable lipids.

40. The pharmaceutical composition of any one of embodiments 18-29, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of an ionizable lipid of Formula (AX).

41. The pharmaceutical composition of any one of embodiments 18-29, wherein the lipid nanoparticle comprises (a) about 2 mol % of DMG-PEG2k; (b) about 25 mol % cholesterol; (c) about 40 mol % sphingomyelin; and (d) about 33 mol % of an ionizable lipid of Formula (AX).

42. The pharmaceutical composition of any one of embodiments 18-29, wherein the lipid nanoparticle comprises (a) about 2 mol % of DMG-PEG2k; (b) about 25 mol % cholesterol; (c) about 40 mol % sphingomyelin; and (d) about 33 mol % of the one or more ionizable lipids.

43. The pharmaceutical composition of any one of embodiments 18-22 and 24-29, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of the one or more ionizable lipids.

44. The pharmaceutical composition of any one of embodiments 18-22 and 24-29, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of an ionizable lipid of Formula (AX).

45. The pharmaceutical composition of any one of embodiments 18-29, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of the one or more ionizable lipids.

46. The pharmaceutical composition of any one of embodiments 18-29, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of an ionizable lipid of Formula (AX).

47. The pharmaceutical composition of any one of embodiments 18-29, wherein the lipid nanoparticle comprises (a) about 2 mol % of DMG-PEG2k; (b) about 25 mol % cholesterol; (c) about 40 mol % DSPC; and (d) about 33 mol % of an ionizable lipid of Formula (AX).

48. The pharmaceutical composition of any one of embodiments 18-29 and 32, wherein the one or more phospholipids comprises a mixture of sphingomyelin and DSPC, the structural lipid is cholesterol, and the PEG lipid is DMG-PEG2k.

49. The pharmaceutical composition of any one of embodiments 18-29 and 32, wherein the ionizable lipid of Formula (AX), the one or more phospholipids comprise a mixture of sphingomyelin and DSPC, the structural lipid is cholesterol, and the PEG lipid is DMG-PEG2k.

50. The pharmaceutical composition of any one of embodiments 18-22, 24-29 and 32, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of the one or more ionizable lipids.

51. The pharmaceutical composition of any one of embodiments 18-22, 24-29 and 32, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of an ionizable lipid of Formula (AX).

52. The pharmaceutical composition of any one of embodiments 18-29 and 32, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of the one or more ionizable lipids.

53. The pharmaceutical composition of any one of embodiments 18-29 and 32, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of an ionizable lipid of Formula (AX).

54. The pharmaceutical composition of any one of embodiments 18-29 and 32, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of an ionizable lipid of Formula (AX).

55. The pharmaceutical composition of any one of embodiments 18-29 and 32, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of the one or more ionizable lipids.

56. The pharmaceutical composition of any one of embodiments 18-29, wherein the lipid nanoparticle comprises about 20 mol % 1,2-distearoyl-sn-glycero-3-phosphocholine (DSPC) and about 20 mol % sphingomyelin.

57. The pharmaceutical composition of any one of embodiments 18-56, wherein the one or more ionizable lipids is compound AX-6.

58. A method of expressing the CAR in an immune cell by administering the pharmaceutical composition of any one of embodiments 18-57.

59. A method of expressing the CAR in a T cell by administering the pharmaceutical composition of any one of embodiments 18-57.

60. A method of expressing the CAR in blood, optionally selected from whole blood or peripheral blood, by administering the pharmaceutical composition of any one of embodiments 18-57.

61. A method of expressing the CAR in spleen by administering the pharmaceutical composition of any one of embodiments 18-57.

62. A method of expressing the CAR in bone marrow by administering the pharmaceutical composition of any one of embodiments 18-57.

63. A method of treating a subject in need thereof, comprising administering to the subject the pharmaceutical composition of any one of embodiments 18-57.

64. The method of embodiment 63, wherein the subject's B cell count is decreased 48 hours post-treatment as compared to pre-treatment.

65. The pharmaceutical composition of any one of embodiments 18-57 for use as a medicament.

66. Use of a pharmaceutical composition of any one of embodiments 18-57 for the manufacture of a medicament for treatment of a disease or disorder in a subject in need thereof.

66. The method of embodiment 63, the pharmaceutical composition for use of embodiment 64 or the use of embodiment 65, wherein the subject's B cell count is decreased post-treatment as compared to pre-treatment.

### INCORPORATION BY REFERENCE

All publications, patents, and patent applications mentioned in this specification are herein incorporated by reference to the same extent as if each individual publication, patent, or patent application was specifically and individually indicated as being incorporated by reference herein.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

LENGTHY TABLES
The patent application contains a lengthy table section. A copy of the table is available in electronic form from the USPTO web site (https://seqdata.uspto.gov/docdetail?docId=US20260325678A1). An electronic copy of the table will also be available from the USPTO upon request and payment of the fee set forth in 37 CFR 1.19(b)(3).

1. A circular RNA comprising:
a. a translation initiation element comprising an internal ribosome entry site (IRES) sequence, wherein the IRES sequence is at least 95% identical to SEQ ID NO: 207 or SEQ ID NO: 217, and
b. an expression sequence encoding a CD19 binding molecule, wherein the CD19 binding molecule comprises a heavy chain variable (VH) domain comprising a VH CDR1 comprising SEQ ID NO: 412, a VH CDR2 comprising SEQ ID NO: 413, a VH CDR3 comprising SEQ ID NO: 414, and a light chain variable (VL) domain comprising a VL CDR1 comprising SEQ ID NO: 415, a VL CDR2 comprising SEQ ID NO: 416, and a VL CDR3 comprising SEQ ID NO: 417.

2. The circular RNA of claim 1, wherein the heavy chain variable domain (VH) comprises an amino acid sequence that is at least 95% identical to SEQ ID NO: 385 and the light chain variable domain (VL) comprises an amino acid sequence that is at least 95% identical to SEQ ID NO: 386.

3. The circular RNA of claim 2, wherein the CD19 binding molecule is an scFv.

4. The circular RNA of claim 3, wherein the scFv comprises an amino acid sequence that is at least 95% identical to SEQ ID NO: 398.

5. The circular RNA of claim 1, wherein the CD19 binding molecule is a chimeric antigen receptor (CAR).

6. The circular RNA of claim 5, wherein the CAR comprises an amino acid sequence that is at least 95% identical to SEQ ID NO: 295 or SEQ ID NO: 298.

7. The circular RNA of claim 1, further comprising a miRNA binding site.

8. The circular RNA of claim 1, further comprising a 3′ self-spliced exon segment upstream of the translation initiation element and a 5′ self-spliced exon segment downstream of the expression sequence.

9. The circular RNA of claim 1, comprising at least one modified A, C, G, or U nucleotide or nucleoside.

10. A pharmaceutical composition comprising:
a. a circular RNA comprising:
i. a translation initiation element comprising an internal ribosome entry site (IRES) sequence, wherein the IRES sequence is at least 95% identical to SEQ ID NO: 207 or SEQ ID NO: 217, and
ii. an expression sequence encoding a CD19 binding molecule, wherein the CD19 binding molecule comprises a heavy chain variable (VH) domain comprising a VH CDR1 comprising SEQ ID NO: 412, a VH CDR2 comprising SEQ ID NO: 413, a VH CDR3 comprising SEQ ID NO: 414, and a light chain variable (VL) domain comprising a VL CDR1 comprising SEQ ID NO: 415, a VL CDR2 comprising SEQ ID NO: 416, and a VL CDR3 comprising SEQ ID NO: 417; and
b. a lipid nanoparticle comprising:
i. one or more ionizable lipids;
ii. one or more phospholipids, in an amount of about 20 mol % to about 60 mol % of the total lipid content of the lipid nanoparticle;
ii. one or more structural lipids; and
iv. one or more PEG lipids.

11. The pharmaceutical composition of claim 10, wherein the lipid nanoparticle comprises (a) about 25 mol % to about 45 mol % of the one or more ionizable lipids; (b) about 30 mol % to about 60 mol % of the one or more phospholipids; (c) about 15 mol % to about 35 mol % of the one or more structural lipids; and (d) about 1 mol % to about 3 mol % of the one or more PEG lipids.

12. The pharmaceutical composition of claim 10, wherein the one or more ionizable lipids comprises a compound of Formula (AX) 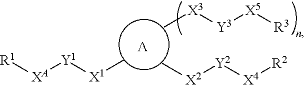
or a pharmaceutically acceptable salt thereof, wherein:
A is selected from an optionally substituted bridged carbocyclic or heterocyclic core selected from the group consisting of: 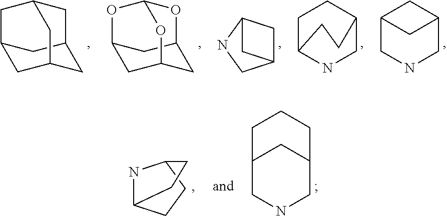
n is and integer selected from 1 or 2;
R¹ is selected from the group consisting of —OH, —OAc, —NR₂, 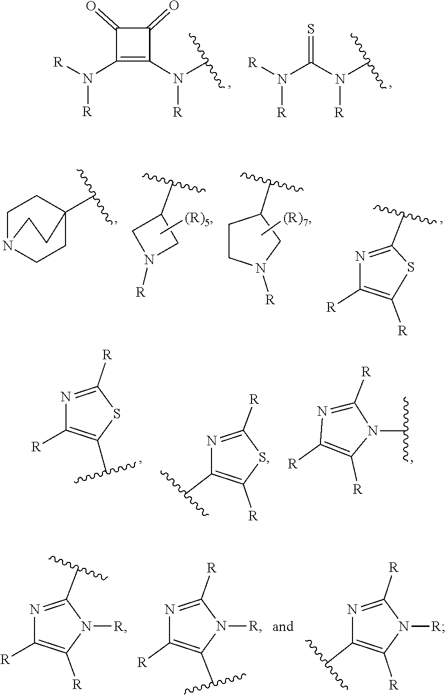
each R is independently —H or C₁-C₆ aliphatic;
X¹ and X^{A} are each independently a bond or optionally substituted C₁-C₆ aliphatic;
Y¹ is selected from the group consisting of 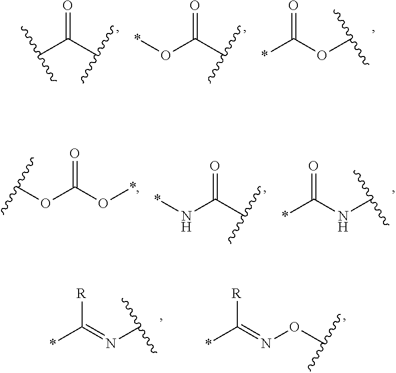
and bond; wherein the bond marked with an “*” is attached to X¹;
each X² and X³ is independently a bond or optionally substituted C₁-C₁₂ aliphatic;
each Y² and Y³ is independently selected from the group consisting of 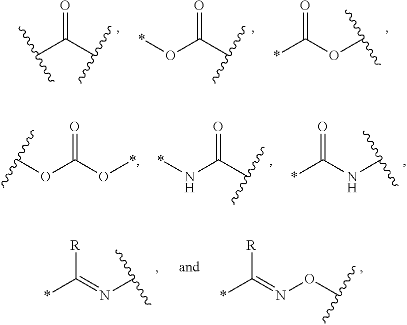
wherein the bond marked with an “*” is attached to X² or X³;
each X⁴ and X⁵ is independently optionally substituted C₁-C₆ aliphatic;
R² is —CH(OR⁶)(OR⁷), —CH(SR⁶)(SR⁷), —CH(R⁶)(R⁷), —R¹⁰, optionally substituted C₅-C₁₈ aliphatic, or optionally substituted C₁-C₁₄ aliphatic-R¹⁰, wherein one or more methylene linkages of R² are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R³ is independently —CH(OR⁸)(OR⁹), —CH(SR⁸)(SR⁹), —CH(R⁸)(R⁹), —R¹¹, optionally substituted C₅-C₁ aliphatic, or optionally substituted C₁-C₁₄ aliphatic-R¹¹, wherein one or more methylene linkages of R³ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁶ and R⁷ are each independently optionally substituted —C₁-C₁₄ aliphatic, —R¹⁰, or optionally substituted —C₁-C₁₄ aliphatic-R¹⁰; wherein one or more methylene linkages of R⁶ and R⁷ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
R⁸ and R⁹ are each independently optionally substituted —C₁-C₁₄ aliphatic, —R¹¹, or optionally substituted —C₁-C₁₄ aliphatic-R¹¹; wherein one or more methylene linkages of R⁸ and R⁹ are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)—, or —C(O)O—;
each R¹⁰ and R¹¹ is independently an optionally substituted cylic, bicyclic, bridged bicyclic, multicyclic or bridged multicyclic C₄-C₁₄ cycloalkyl or optionally substituted cylic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

13. The pharmaceutical composition of claim 10, wherein:
(a) the one or more phospholipids are selected from 1,2-distearoyl-sn-glycero-3-phosphocholine (DSPC) and egg sphingomyelin, and combinations thereof;
(b) the one or more structural lipids are selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate, and combinations thereof; and/or
(c) the one or more PEG lipids is selected from DMG-PEG2k, DSPE-PEG2k, DSG-PEG2k, DMPE-PEG2k, DPPE-PEG2k, and mixtures thereof.

14. The pharmaceutical composition of claim 10, wherein the phospholipid is sphingomyelin, the structural lipid is cholesterol, and the PEG lipid is DMG-PEG2k.

15. The pharmaceutical composition of claim 12, wherein the ionizable lipid is a compound of Formula (AX), the phospholipid is sphingomyelin, the structural lipid is cholesterol, and the PEG lipid is DMG-PEG2k.

16. The pharmaceutical composition of claim 12, wherein the lipid nanoparticle comprises about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of an ionizable lipid of Formula (AX).

17. The pharmaceutical composition of claim 10, wherein the one or more ionizable lipids is compound AX-6.

18. The pharmaceutical composition of claim 10, wherein the lipid nanoparticle comprises (a) about 2 mol % of DMG-PEG2k; (b) about 25 mol % cholesterol; (c) about 40 mol % sphingomyelin; and (d) about 33 mol % of an ionizable lipid of compound AX-6.

19. A method of expressing CAR in an immune cell by administering the pharmaceutical composition of claim 10.

20. A method of treating a subject in need thereof, comprising administering to the subject the pharmaceutical composition of claim 10.


SEQUENCE LISTING (435 sequences)

SEQ ID NO: 1 - protein, 5 aa, synthetic construct
GGGGS

SEQ ID NO: 2 - DNA, 294 bp, Anabaena sp.
  source 1..294: mol_type = genomic DNA
gaatggtagacgctacggacttaaataattgagccttagagaagaaattctttaagtggatgctctcaaactcagggaaacctaaatctagctatagacaaggcaatcctgagccaagccgaagtagtaattagtaagttaacaacagataacttacagctaatcggaaggtgcagagactcgacgggagctaccctaacgtcaagacgagggtaaagagagagtccaattctcaaagccaataggcagtagcgaaagctgcgggagaatgaaaatccgttgaccttaaacggt

SEQ ID NO: 3 - DNA, 361 bp, Hypocrea pallida
  source 1..361: mol_type = genomic DNA
gagggcaagtctggtaaacgttcgctggtgccagaaatagccagccctgtaaaatggctagtcgagtgctcgatctgtagcgggtgagcaccggcgagacaacctggatcggggaagcctaagggacctgtcacagggtcctatggtgatcccgagcagagccgcaaggcctgtgtagagcacgctaaggtgtcggtcccgtcctctgcggcggggcttaaggtacgtgccaatcccctgcgaaagctgggccctagggagtagcacccgctgtgcgaagctctaggggacctgtgcccgccgtggcgcaggccgtccgacagtcgtcagactgctcactataatggccagcagccgcggt

SEQ ID NO: 4 - DNA, 324 bp, Bulbithecium hyalosporum
  source 1..324: mol_type = genomic DNA
gagggcaagtctggtgaactgttggtgccagagatagctgcggcccgcaacggcagctagtgggtgctcgatctgtgacgggtgagcacctgcgagacaacctggatcgggggaccctaagggcgccacgtgcgcctatggaaatcccgagcagagccgcaaggcctgtgtagagcgcgccaaggtgtcggtctgacttctgtcaggcttgaggtacgtgccgatccccagcgaaagctgggcccagtggactagcacccgtcgtgcgaagccgctggggagtccgtgatggactttagcgacaaatcggccagcagccgcggt

SEQ ID NO: 5 - DNA, 320 bp, Mycoarachis inversa
  source 1..320: mol_type = genomic DNA
gagggcaagtctggtgaactgttggtgccagatagagccgcggccctgaacggcggctagtcgagtgcccgtcagtgacgggtgggcaccggcgagacaacctggatcgggggaccctaagggccgagaggcctatggaaatcccgagcagagcctgtagaggcctgtgtagagcgcgccaaggtgtcggtccgtccccgcggggacgggcttaaggtacgtgccgaccccccgcgaaagcggggcctaggggatgagcacccgtcgtgcgacgcccctggggagcatacgggctatgtataacggccagcagccgcggt

SEQ ID NO: 6 - DNA, 320 bp, Allomyces macrogynus
  source 1..320: mol_type = genomic DNA; note = Mitochondrion
tagttgaatttgtttaacaagttccggagaatagtaatattctcatgaaaatcgggtgaattgtcaaggagacctcctacaatggagatgacttgcagcgaagcttatatcagtgcttaatagatataagaacgttcaacgactaatcggtgagtagcactaacaataaaccggacactaggtggtgagtacaaccaacaataatccacccacgagcgcccgacaaccataattatggttgatgacatagtctgaacaggcaggtgactgtctgaagagaaatttaaacgatttctcgataacataattgttgtttgggg

SEQ ID NO: 7 - DNA, 411 bp, Geosmithia argillacea
  source 1..411: mol_type = genomic DNA
ggcaccacaaggcgtaaaccattgctacagccgcagcaactctgcgccgaacagcagcccgaaagggtgatgtggtgccctctaagcggtaaatgctagtctgcccccgggcaggcgacacgctcaaattgcgggaaacccctaaagctcccagctaccaagccgccggtcgaaagacggcggtggccaggttaattgcctcgggtacggtcacaacgctgggagatgcatgggcgatccgcagccaagcccctacgtccccccggggatacggggaaggttcagagactagatgggcgtgggttgaccgggaggggggcgtgtccccccccgggcggcttaagatatagtccggcaccgggagagatcccggtggtcaggtccacctgtaacacgggagcctgcggctta

SEQ ID NO: 8 - DNA, 440 bp, Stephanoascus ciferrii
  source 1..440: mol_type = genomic DNA
aggctgaaacttaaaggaattgacggaagggcaccaccaggagtaaacttttacagctagccgcagtaactctgctccaaaaagcagcctgaaagggttagtggtgtcttcttttaattgctagtcgatatttttgtatcggcaacacgctcaaattgcgggaaattcctaaaggctttattaccaagctaacattgaaaggtgttggtggccgagcgaaatgccctgggtatggtaaaaacataaagcatgttacaatggatgatccgcagccaagctcctaagttcttgaaaaaaggatatggagaaggttcagagactaaatgggtgtgggtgtttgtaacgcttaagatatagtccgtccatgactgaaaagtcaatggtggtaaccttaaaacgggagcctgcggcttaatttgactcaacacggggaaactcac

SEQ ID NO: 9 - DNA, 391 bp, Coxiella burnetii
  source 1..391: mol_type = genomic DNA; note = RSA 493
tggggttagccctcgggcgaagctcttgatcgaagccccggtaaacggcggccgtaactataaattaaccgttgtagttacgtaaataaacttggctatatgctggaaactccgagtatcctccagtactcatctcaactaaaattgaggtagtaaaaatctgcgaggtgcggacaatcagcaggaaagactaatctttttgattagaatcctcagagactaatacgccgagcgtacttagaaaaaatccattctaagtgtgatgatagagtccgatctctatagcgatatagagggttttcaaaagaaacctgttaatcattatcccatgattaacataacgtcaataaacggtcctaaggtagcgaaattccttgtcgggtaaaaaatt

SEQ ID NO: 10 - DNA, 267 bp, Agrobacterium tumefaciens
  source 1..267: mol_type = genomic DNA
gagcacaggattcctaaggtaaattgggggttgcgcccggaaacgacgcaatcgatctgctcaaagtcggggaaagcttcgctggtcttccagtatgccaatcccgagccaagctccggagaaatccggtgaaggtgtagagactggatgggcagcacctaaaggccgcaaggtccacggtgaagggacagtccagaccacgaacgcccttatccgattgagatgggcggcggcgaaagccgaagcggtatgaatcctggggtcgga

SEQ ID NO: 11 - DNA, 408 bp, Ankistrodesmus stipitatus
  source 1..408: mol_type = genomic DNA
atgggtgaaagcactattgcccatcatagcagctgtccacaaagcagtctgctagtcgtcgttgcaccgctttgcaaggcgcaacgcacggcgagaccgtcgaattgcggggacatccttacagctcaagctaccaacttgtcgcagaaatgctggcaagggccggggtaatgacccagggtatggtaaaaacgcttgagattggataatccgcagccaagcaccttacccggattcaacgtccggaaaggtgaaggttcagagactaggtggcggtcggtcacgagacgtggcttaagatagagtccgttcgcgctgaaaagcgttcgtcgggaagaaagccttagcaaggccggagccagacgaagggtagccattattggctgcctcgaggtacacgggtggtgc

SEQ ID NO: 12 - DNA, 445 bp, Monacrosporium leptosporum
  source 1..445: mol_type = genomic DNA
tttagaggaagtaaaagtcgtaacaaggtttgaagtatgacccttctgtggtgacacttacgagaagcctttgcggccccgcaaggggtaccggccgcgactataaatgaaaggcaagccggcattaaatcgcaagtcagctcagctggctacactttcgaattgcgggaaacccctaaagccgacctctaccaactgttcagggaaacctggatggggcctgtgctaacatcacagggtacggtaacaatgaggaggatatatgggcaatccgcagccaagtccctaaggctctcgagctatgggaaaggttcacagactaagtggaagtgggttggggcggttgtaatccgccccggcttaagatatagtcgggctgtccgcgagagcagacaggatgtcacaaatcaaccgttccgtaggtgaacctgcggaaggatcatta

SEQ ID NO: 13 - DNA, 277 bp, Nostoc sp.
  source 1..277: mol_type = genomic DNA; note = F.
agataactgagccttgaaggagaaatccctcaagtggaagctctcaaactcagggaaacctaaatctggtgacagacatggcaatcctgagccaagcccaaaaattttagatttgcgattagtcttaaattcaaaatctaaaatccaaaattacgggaaggtgcagagacccgacgggagctaccctaacgtgaagtcgagggtaaagggagagtccaattctcaaaacctgatttggcgattgccagcaagtagcagtgaaaactgcgggagaatg

SEQ ID NO: 14 - DNA, 272 bp, Nostoc sp.
  source 1..272: mol_type = genomic DNA; note = C.
agataactgagccttgaaggagaaatccctcaagtggaagctctcaaactcagggaaacctaaatctggtgacagacatggcaatcctgagccaagccgaaaaaagtcgtgagtgcagagtaaattaaaactcttaactcctaactattcggaaggtgcagagacccgacgggagctaccctaacgttaagtcgagggtaaagggagagtccaattctcaaaacctgatttggctattgctatcaggtagcagtgaaaactgcgggaggatg

SEQ ID NO: 15 - DNA, 439 bp, Monoraphidium terrestre
  source 1..439: mol_type = genomic DNA
ggagaagtcgtaacaaggtttccgttgaattcactagcggataacagctgccctgaaaggggtggctagtcattgatagctcatgatggtgagggctttcaattggcaacactgtcgaattgcgggaaattccttacagttctcactaccaagccttcgtggaaacacagtcggtggcctagggtaatcacctagggtatggtcaaaacgtgagggattggaaaatccgcagccaagctcctaaaggcgacatgacctatggagaaggttcagagactaggtggcagtgggctagttgctgcgtgcaattagtttaagatagagtccggccctaccgtgaggtagcctcaagacgaaaggactaaccagcccagagcttgaggagctgatgtgttacgtatcagtgggatcaaccgaggtgaacctgcggaaggatcat

SEQ ID NO: 16 - DNA, 450 bp, Arxula adeninivorans
  source 1..450: mol_type = genomic DNA
cagaaaagttaccacagggatgtgctaagtctattaagacaaatttacaacataacgtcccaggcatcgactcccattgatgcctagtccggctatagcttacttgtgcttgctatagttagctctctagaagagtgtaagggcaacacgtctggatgcggggaactctcgctaggtctttggtaccaaacattggcggtaacaccccaatgtagagtctgttggggcagacgggtaaaaatccaaagaatagagacaatccgcagctgacctggccaaaagtatatttacaaagtagctaggcagttcaacgctcgctaagatgtgggttgaccattaatggtcggcttaaggtacgggctacgccacccgagagggtgcaactacctaccgggcgcacaacctgagaagtagttggtcatggtagtacggaactggcttgtggcagtc

SEQ ID NO: 17 - DNA, 411 bp, Penicillium oblatum
  source 1..411: mol_type = genomic DNA
gaaagttaggggatcgaagacgatttaaggaagcctcccggcctggtttccgtggaaatcaggccggagatggtatttggtcgtccaaagtaagcctgaaaggacttgctagtctcggccgtggccgaggcgacaccgtcaaattgcggggacctcctaaagcctggactaccaagccgacgccgaaaggcgccggtggccggggtaatgacctagggtatggtaacaacgtccgggatgtgacaatggacaatccgcagccaagcgctaccgcccctccggggaccacgcgtgcagttcacagactagatgtcggtgggggatccgtctcctaagatatagtcgaggcccagcgcgaaagcctgggagtatccgcagataccgtcgtagtcttaaccataaactatgccg

SEQ ID NO: 18 - DNA, 282 bp, unidentified
  source 1..282: mol_type = genomic DNA; note = Staphylococcus phage Twort
aactactgaaagcataaataattgtgcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttattt

SEQ ID NO: 19 - DNA, 431 bp, Cryptodiaporthe corni
  source 1..431: mol_type = genomic DNA
gagcatgggggcgaaagactaatcgaactgttgacgcacgagagtgtgggcacaccgtatgaaaattcgttttcgcgggctgctaggcagttaagagtccggccgtaaggcagggctagtaggcttcagagaggcctgcgacaccgtcaaattgcggggacaccccattagaaagctgcgctaccgcctacggctcgaaagagctgtcaggcaccaggtgtaacggcctgggtatggtaataacgcgtgtacgcaaggggcaatccgcagccaatccctaacccacctctataggtggtatggaggcagttcacagactagacggcggtgggcaccgttacagaaggtgcttaagatatagtcgagtcccgcctagcaataggtgggtagtttaacgctagtagctggttaccgccgaagtttccctcagg

SEQ ID NO: 20 - DNA, 267 bp, Pleurocapsa sp.
  source 1..267: mol_type = genomic DNA
acggacttaaaaaaattgagccttggcagagaaatctgtcatgcgaacgctctcaaattcagggaaacctaagtctggcaacagatatggcaatcctgagccaagccttaatcaaggaaaaaaacatttttaccttttaccttgaaaggaaggtgcagagactcaacgggagctaccctaacaggtcaagctgagggtaaagagagagtccaattctcaaagccagcagatggcagtagcgaaagctgcgggagaatgaaaatccgt

SEQ ID NO: 21 - DNA, 447 bp, Spizellomyces punctatus
  source 1..447: mol_type = genomic DNA
cgttgattacgtccctgcccttttccttgatggtacaaggggctaaatgtgctcccgaaaggaatgcgcaagtcttcagtggcaccgaaggcaacactgtcaaattgcggggaagccctaaagctttgtctaccaagcagggatggaaacatttctgtggccagggtaatgacctcgggtatggtaaaaacggcaaagatagaacaatgggtaatccgcagccaagtcctaaacttgcacccagctggggcaagcatggatgcagttcacagactaaatggcagtgggctctgcgatagagattattgcggggcttaagatatagtcggacccaccgcgagagcggttctctagatgaagctcagtgatttccaggtgcctgaagaactgatcctgagcccgagctagagtgtaaagggagcatcgttgtacacaccgcccgtcgct

SEQ ID NO: 22 - DNA, 359 bp, Chlamydomonas moewusii
  source 1..359: mol_type = genomic DNA; note = Z17234
caggcttagttgtaggcctcctattaggcaactaatgggctaacattgggtgaattgcatgaaagctaaggcgtaacgctatgctaacctgcagcgaattacatcgactgatattaatataaaaatagatgtacacgttcaacgactagcgagtgatgtaaatcagtcaaataatctcgccacgagcgcccgacatcttgatatttacttagacccccgcttaagtaaatatggccaagatgatgacatagtctgagcaacgtggaaacacgttggagccaaagataaaaaactttggcacatttctgaaacatgaaatgttcacgttaaaaaaaataaaaacgtgaaaaacataactg

SEQ ID NO: 23 - DNA, 439 bp, Kirchneriella aperta
  source 1..439: mol_type = genomic DNA
ggaattgacggaagggcaccaccaggcgttaaaatagctctagcgccttagagtctcctcgagaggggaggctagtctgtagtggctcacatcggtgagagcttctatcaggctacactgtcgaattgcggggaaatccttatagctcaagctaccaagccaggatggaaacattctggtggcctggttaatgacccagggtatggtaacaacgcttgagattggacaatccgcagccaagcttcccaccaggtaatgctgaggaagacggttcagagactaagtggcagtgggcgggtgcaaacctgcttaagatagagtccgtccctaccgaaaggtagctctgagaggaaaggcccaaccagcctaaagctcagagagcttgcttgagcaagtgggagtaaacgggagcctgcggcttaatttgactcaacacggg

SEQ ID NO: 24 - DNA, 384 bp, Pneumocystis carinii
  source 1..384: mol_type = genomic DNA
taaacggcgggagtaactatgactcaccttctgagggtcatgaaagcggcgtgaaaacgttagctagtgatctggaataaattcagattgcgacactgtcaaattgcggggaagccctaaagattcaactactaagcagtttgtggaaacacagctgtggccgagttaatagccctgggtatagtaacaatgttgaatatgaatcttttgcgagatgaaatgggtgatccgcagccaagtcctaagggcatttttgtctatggatgcagttcaacgactagatggcagtgggtattgtaaggaattgcagttttcttgcagtgcttaaggtatagtctatcctctttcgaaagaaagagtatattgtgctcttaaggtagccaa

SEQ ID NO: 25 - DNA, 400 bp, Panellus stipticus
  source 1..400: mol_type = genomic DNA
ccaccaggtgtaaacttgttagccacagttaactctgcactaaaaagcagccctaacgggtgaggtggttcagccctagagtgcgcaatgactcttaaatctaaatgctagtctgtgcatccttgtgttgcacgggcgagaccttcaaactgcgggaaactccttagagctttcactaccgctgctatcactgaaaagtgtagtctgcaccagggtaatgacctcgggtatggtaataacgtgaaagattggacaatccgcagccaagtccctaaaggtagttattgacctacgggaaaggttcagagactagatggaggttggtcaactaagttggcttaaggtatagtccgtctctgcatgaaagtgcaagaggtttgaaatgggagcctgcggctta

SEQ ID NO: 26 - DNA, 396 bp, Dunaliella parva
  source 1..396: mol_type = genomic DNA
accaggcgttaaaaagcagactcagcgcctaagagtcagtgggaaaccattggctagtgcttgggtttcattactcaagtgcaacactgatcaaattgcgggaaagccctaaagctttgctaaccaagctaagtgtgaaagcactcagtggccgggttaaagacctcgggtatggtaaaatcagcaaagatgcaacaatgggcaatccgcagccaagctcctgaagccttataggcaatggagaaggttcagagactaaatggcagtgggccaacttgttggcttaagatatagtccgtcccagctgaaaggctgtctgctagaggaagacctttctaggtctgagagctagtagagggtaggagaagtctctcctacctggaggaaacgggagcc

SEQ ID NO: 27 - DNA, 217 bp, Azoarcus sp.
  source 1..217: mol_type = genomic DNA
ctcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaaacgctatggtgaaggcatagtccagggagtggcgaaagtcacacaaaccggaatccgt

SEQ ID NO: 28 - DNA, 424 bp, Ditopella ditopa
  source 1..424: mol_type = genomic DNA
gagcatgggggcgaaagactaatcgaactgttagctactaggcagttaagagtctcccagtaatgggaggctagtaggctctttttcagagtttgcgacaccatcaaattgcggggacaccctgttataacgctatgataccgcgcctcgtggaaacacagagagtagcactaggtgtaacggcctaggtatggtaaaaagtcattagctaggggcaatccgcagccaagccctacgtcttcggatatgggtgcagttcacagactaaacgttggtgggcttagaatctgtattctaggcttaagatatagtcgaatccgcctagtaataggtgggtatcaaatgaataggtgcaaaaacttgtttatttgactacagcagaaatgctagtagctggttaccgccgaagtttccctcaggatag

SEQ ID NO: 29 - DNA, 264 bp, Anabaena sp.
  source 1..264: mol_type = genomic DNA
cggacttaaataattgagccttagagaagaaattctttaagtggatgctctcaaactcagggaaacctaaatctagctatagacaaggcaatcctgagccaagccgaagtagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatgaaaatccg

SEQ ID NO: 30 - DNA, 442 bp, unidentified
  source 1..442: mol_type = genomic DNA; note = chlamydomonad sp. NDem2 24P 3d
gttatagtttatttgatggtactcttactcggataaccaagcaaaaggttagtccgtgtcagtttaaacgactggcaagtcgattctattggatcggcaacaccatcgaattgcgggaaagtccttagagccagtgctaccaagctggagtggaaacacaccggtggccagggtaatgacctcgggtatggtaaaaacgcactggattggataatccgcagccaagctcctaagggtagcattgcctatctatggagaaggtccaacgactaagtgttggtgggctggtagcaaaccggcttaagatatagtctggccctgctgagaggcagccttgggattaaaggcttaaacagcccggagcccaaggggttggagcacatgctctgaccggaacaaccgaaccgtagtaattctagagctaatacgtgcgtaaatcccg

SEQ ID NO: 31 - DNA, 400 bp, Penicillium sabulosum
  source 1..400: mol_type = genomic DNA
gaaagttaggggatcgaagacgatttagggccctcccggcgtccgccgacgccgggggatggtatttggtcgtcaaaagtaagctgaaaaggtcttgctagtcccggccgcggccggggcgacaccgtcaaattgcggggactccctaaagcctgaactaccaagccgacgccgaaaggcgccggtggccggggtaatgacctagggtacggtaacaacgttcaggatgtgccaatgggtaatccgcagccaagcgctaccgcccgtcccgggccacgcgtgcagttcacagactagatgtcggtgggggatccgtcccctaagatatagtcgagccccggagcgaaagcccggggaggatccgcagataccgtcgtagtcttaaccataaactatgccg

SEQ ID NO: 32 - DNA, 429 bp, Ustilago maydis
  source 1..429: mol_type = genomic DNA
accaggagtaaactttcacagctagccgcagtgtctctgctccaaaaagccgcctgaaagggtcggtgcgtgttcctatttgaaataatggctagtctgatgcgaatcaggcgacacatccaaattgcgggaaactccttagagctctagctaccgcggccccatcgtgagattagggtgcagcaccaggttaatggcctcgggtatggtaaaaacgctagagattggacaatccgcagccaagcctcttcgcaagaagtggaaggttcagagactaaatggttgtgggtggcagacggtttcgcgaatgtatgaatcaatcataaacacgtgaaggaagtctgctgcttaagatatagtccgagccacagggaaaccttgggaattttggcaacaaaccgaaacagataaataacatcgggagcctgc

SEQ ID NO: 33 - DNA, 442 bp, Pleurastrum paucicellulare
  source 1..442: mol_type = genomic DNA
ccaggcgtaaaaccaagccgtttggctctgcgccagtcgggtgtgaggagggcctcaaacaagctcctcacacgagaaacccggctagtcctggtgcggcttgcatcgcgccgccgcacctgggcaacatcatcgaattgctggaaacccctaaagctcttggtaccaacctgcatgggaaaccgtgcgggggcccactccggggaggttataatccaagagatgcaacaatgggcaatcagcagcgaagaccctgtcggttgggacttgcgtcccggcccatgggtaacgttcagagactaagtggtgatgggccccgatgccacccggcctcggggcttaagatatagtccggccccggcgaaagccgatgtcgagatgagggggtgcgaccccagagctcggcagtagggatcagagttcctgctctaatgtnggagcg

SEQ ID NO: 34 - DNA, 420 bp, Macropharyngomonas halophila
  source 1..420: mol_type = genomic DNA
agtacaaggaagcggaaggctataacaggtttaaaggcttgagtggcgcgaaagcgaccgctttgtggctacaaagtcgcgacaccgtcaaattgcgggaacaccctaaagcttcaactaccaagcgtatgtggaaacacttgcgtggccagggtaatgacctcgggtatggtaaaaacgttgaagatgaaacaatgggcaatccgcagccaagccctaaagggatttttcctaagggtgcagttcacagactagatgtcggtgggtagagtcgtactctgcttaagatatagtcgaggccaccgtgaaaacggtgcacagtggaggaaattcggggtaactcctggatggagagccgctgtgggtagacaaaagtctactggtaacctgctgtaatgcccttagatgttctgggctgca

SEQ ID NO: 35 - DNA, 298 bp, Nostoc sp.
  source 1..298: mol_type = genomic DNA; note = A.
agataactgagccttggaggagaaatccctcaagtggaagctctcaaactcagggaaacctaaatctgacaacagacatggcaatcctgagccaagcccaaaaattttagatttgtgattttagatttacgattagtcttcaatccaaaattcaaaatctaaaatccaaaattgcgggaaggtgcagagacccgacgggagctaccctaacgttaagtcgagggtaaagggagagtccaattcttaaaacctgagctggctattgccattaggtagcagtgaaaactgcggaagaatg

SEQ ID NO: 36 - DNA, 322 bp, unidentified
  source 1..322: mol_type = genomic DNA; note = Nostoc sp. Nephroma resupinatum
agataactgagccttgaaggagaaatccctcaagtggaagctctcaaactcagggaaacctaaatctggttacagacatggcaatcctgagccaagcccaaaagttttagatttgcgattttagatttgcgattaatcttcaatccaaaattcaaaatcgttcgactgagcgaagccgaagtctaaaatccaaaattgagggaaggtgcagagacccgacgggagctaccctaacgttaagtcgagggtaaagggagagtccaattctcaaaatctgatctggctattgtcatcaggtagcagtgaaaactgcgggagaatg

SEQ ID NO: 37 - DNA, 331 bp, Nostoc sp.
  source 1..331: mol_type = genomic DNA; note = J.
agacgctncggacttaaataattgagccttgaaggagaaatccctcaagtggaagctctcaaactcagggaaacctaaatctggtgacagacatggcaatcctgagccaagccgaagaaagtcctgagtcatgagtgctgagtgctgagtaaatttaaaactcttaactcctaactcctaactcataactgttcggaaggtgcagagacccgacgggagctaccctaacgttaagtcgagggtaaagggagagtccaattctcaaaatctgatctggctattgtcatcaggtagcaatgaaaattgcaggagaatgaaaatccgttgaccg

SEQ ID NO: 38 - DNA, 350 bp, Cordyceps capitata
  source 1..350: mol_type = genomic DNA
aattggagggcaagtctggtgaacgtcgctggcgccagagatagccgccctcggtaaacaaccggctagtcgagcgctcgttgcgtgacgggtgagcgccggcgagacaacctggatcggggaaggctaagggccccgcgggcctacgctgatcccgagcagagccacgcccggtggcctgtgtagagcgcgccaaggtgtcggtcccgaggtctcctcggggcttaaggtacgtgccaatcccctgcgaaagcagggccctgcggaacagcacccgtcgtgcgaagccgcagggagcccccctccccgggggggcgtgtgctacaggccagcagccgcggtaattccag

SEQ ID NO: 39 - DNA, 270 bp, Nodularia sp.
  source 1..270: mol_type = genomic DNA
acggacttagaaaactgagccttgatcgagaaatctttcaagtggaagctctcaaattcagggaaacctaaatctgtttacagatatggcaatcctgagccaagccgaaacaagtcctgagtgttaaagctcataactcatcggaaggtgcagagactcgacgggagctaccctaacgttaagccgagggtaaagggagagtccaattctcaaagccgaaggttattaaaacctggcagcagtgaaagctgcgggagaatgaaaatccgt

SEQ ID NO: 40 - DNA, 436 bp, Dactylella copepodii
  source 1..436: mol_type = genomic DNA
gtcgtaacaaggttgaagtaagatccttctacagtgacacttacgagaagcctttgtggccccgcaaggggtacctgccgcgactataaacaatgcaagtgggtattaaatcgcaagtcagctttagctggccacactttcgaattgcggggaaaccctaaagctcaccataccaaccagcctgggaaatcaggttggggcccgtgctaactccacgggatatggtaataatgggatgagatgtaacaatgggcaatccgcagccaagtccctaaggtcttgtccagactacgggaaaggttcacagactaagtggaagtgggccggagtttgtaccctccggcttaagatatagtcgggctgcctgtgagagcaagtgggaagtcacagttgcacttcaaaccgttccgtaggtgaacctgcggaaggatcatta

SEQ ID NO: 41 - DNA, 299 bp, Prochlorothrix hollandica
  source 1..299: mol_type = genomic DNA
agacgctgcggacttagataaactgggccttggtggagaaatccgcgaagtgtaagctctcaaattcagggaaacctaaggccaagggctagggcaatcctgagccaagctgagggatcaaccacccctaggggcgttgaccttcggaaggtgcagagactcgacgggagctaccctaacaggccgtctgccaaggcaggcgatcgctgagggtaaagggagagtccaattctcaaaacctctggcaccacgtcaggggcaacagtgaaagctgtgggagaatgaaaatccgttgaccg

SEQ ID NO: 42 - DNA, 392 bp, Protomyces pachydermus
  source 1..392: mol_type = genomic DNA
ttgacggaagggcaccaccaggagtaaacgtttatgtcgcagttaactctgctccgaaaagccactcgaaagagtgtggtgtccttagaggctagtcagtccgctggcaacacaatcaaattgcgggaaactccttagagctcaagctaccgctgctgcccagagatgggctgtggcaccaggttaacgacctcgggtatggtaacaacgcctgagattggacaatccgcagccaagctccctcatgggaagaaggttcagagactagatggttgtgggttgaacacgtttatctgaagtatttgaacggttcggcttaagatatagtccaatccaatgtggaaacgcactggtctgaaagatagaaaatgggagcctgcgcttaatttgac

SEQ ID NO: 43 - DNA, 430 bp, Monacrosporium bembicodes
  source 1..430: mol_type = genomic DNA
gtcgtaacaaggtttggaatataagatccttccgtgacgcttatgagaagcctttgtggccccgcaaggggtacccgccgcgactgtaaaaaaggatgcaggtattaaatcgcaagtcagcctttggctggccacactttcgaattgcggggatgccctaaagccctcctctaccaaccaagctgggaaacctgcttggggcccgtgttaacggcacggggtacggtaacaacgaggttggataggtgggtaatccgcagccgagtccctaaggcaagccgctatgggaaaggttcacagactaagtggaagtgggctggatgctccagcttaagatatagtcgggctgttggcgagagcgaacagatctgtcacttcatcatgattagtgcaaaaccgttccgtaggtgaacctgcggaaggatcatta

SEQ ID NO: 44 - DNA, 338 bp, Tilletiopsis oryzicola
  source 1..338: mol_type = genomic DNA
gaggcaataacaggttaacctatcacaggcctgtaacagctgcgaggcagctagtccagcgtcgacgtctgcatttggggcagatggggtctgtttagaccttggcgcgggcgacacaacctggtacaggaaacgccccggcagcaatgccaggccgatcctgtggcgagctctgcgaggagccgtcgcaacgtgcggaaaggtgtgggtctttctaggaaaggcttaaggtacgtactaatccccggggaaacccggcctctgcgagaagagcccaaactctatgcgcagggggaggcaacggtgttgccttggtatacatgctgtgatgcccttag

SEQ ID NO: 45 - DNA, 382 bp, Protomyces inouyei
  source 1..382: mol_type = genomic DNA
acggaagggcaccaccaggagtaacgtttatgtcgcagttaactctgctccgaaaagccactcgaaagagtgtggtgtccttagaggctagtcagtacgctggcaacacaatcaaattgcgggaaactccttagagctcaagctaccgctgctgcccagagatgggttgtggcaccaggttaacgacctcgggtatggtaacaacgcttgagattggacaatccgcagccaagctccctcatgggaagaaggttcagagactagatggttgtgggttgaacacacgtttaattgaaacagttcagcttaagatatagtccaatccagtgtggaaacgcactggtctaaaagatagaaaatgggagcctgcggcttaatttga

SEQ ID NO: 46 - DNA, 442 bp, Artomyces pyxidatus
  source 1..442: mol_type = genomic DNA
ttgacggaagggcaccaccaggagtaaacgcaacagcttagccgcagtggctctgctccgaacagcagcccgaaagggtgaggtggtttaaaagccttgaggttcgatccgaggacctctaaagcgaaatgctagtctccctcgtggggggcgacaccttcaaattgacggggaactcctaaagctcagagcaccaagcttcgttccgaaagggcggggtggccaggttaatagcctcgggtacggtaaaagactctcgagatgttacaatggacgatccgcagccaaggccctacggcttcccttcgcggggtgagctacgggacaggttcagagactagatggaggtgggtcgtcgcgcgtcgacggcttaaggtatagtccgttcgcgagcgaaagctcgatgaggtttaggtaaacccgcctttgacaaaacacggga

SEQ ID NO: 47 - DNA, 270 bp, Nostoc sp.
  source 1..270: mol_type = genomic DNA; note = B.
agataactgagccttgaaggagaaatccctcaagtggaagctctcaaactcagggaaacctaaatctggtgacagacatggcaatcctgagccaagcccaaaaattttagatttgcgattagtcttaaatctaaaatccaaaattgcgggaaggtgcagagacccgacgggagctaccctaacgttaagccgagggtaaagggagagtccaattctcaaaacctgagctggctattgccatcaggtagcagtgaaaactgcgggagaatg

SEQ ID NO: 48 - DNA, 447 bp, Fusarium solani
  source 1..447: mol_type = genomic DNA; note = f. sp. phaseoli
ggcaccaccaggggtaaactacaacggtttctcgaaagccgcagttaactctgctcctaaaagccgctctaacgagttgatggtgttccctggctcaggcctaaactaatggctagtctcctcggaggcgacatcctcaaactgcgggaaactcctaaagctcacactaccaagcaggcctcgaaagagagctctgtggccggggtaacgacctagggtacggtaaaaacgtgtgagatgctacagtggacgatccgcagccaagcccctacgtgcgccagcatacggggaaggttcagagacttgacgggggtgggtgggctacaggcctgcctaagataaagtccggccccgcgtgaaagcgctgggggttacaactgcttctgcactattcgaaatactgcatcgaggccccagtaaaacgggagcctgcggcttaatttgact

SEQ ID NO: 49 - DNA, 298 bp, Nostoc sp.
  source 1..298: mol_type = genomic DNA; note = I.
agataactgagccttgaaggagaaatccctcaagtggaagctctcaaactcagggaaacctaaatctggtgacagacatggcaatcctgagccaagcccaaaaattttagatttgtgattttagatttatgattagtcttcaatccaaaattcaaaatctaaaatccaaaattgcgggaaggtgcagagacccgacgggagctaccctaacgttaagtcgagggtaaagggagagtccaattctcaaaacctggccttgctaaagccatcaggtagcagtgaaaactgcgggagaatg

SEQ ID NO: 50 - DNA, 375 bp, Pneumocystis carinii
  source 1..375: mol_type = genomic DNA
aactatgactcaccttttgagggtcatgaaagcggcgcgaaagtgttagctagtgatccgaaaaataaattcgggttgcgacactgtcaaattgcggggagtccctaaagattcaactactaagcagcttgtggaaacacagttgtggccgagttaatagccctgggtatagtaacaatgttgaatatgactcttaattgaggaaatgggtgatccgcagccaaatcctaaggacattttattgtctatggatgcagttcagcgactagacggcagtgggtattgtagagatatggggttatttatggccttatctacaatgcttaaggtatagtctaatctctttcgaaagaaagagtagtgtgctcttaaggt

SEQ ID NO: 51 - DNA, 442 bp, Trebouxia gelatinosa
  source 1..442: mol_type = genomic DNA
aggtttccgtaggtttnantatgcggataaaagcaggcttgaaaagagtttcctgctagtacctaactttttaagtaaggtgcgacattgtcaaattgcctggaaatcctgtcaagcccctggtaccgctcatcttgaggaaactctggtgtgcacctcggtgaaagtcgagggtatggtaataatccagcgggataaggacaataggcagccaactgctaaagancccgaaggccctcangcaggcagtccacagactaaatggcaatgggtcaaggagcattaactgctcattggcttaagatatagtcggtcctcgctgaaaagcggccaatgagaggaattctctttgagaaggagagctcattggagtttgggtaatactctgcaatgcaaattgtatgagggcaataagcctccaaactgggtgaaacgaggtgaa

SEQ ID NO: 52 - DNA, 420 bp, Bionectria aureofulva
  source 1..420: mol_type = genomic DNA
aattgacggaagggcaccaccaggggttaacagaccgcagtcgctctgcccctaaaagcagccgggaaactggttaaggtgggagtccttggccggaaggccgaaaactgctagtcatcgaacgatggcaacaccctcaaactgacgggaacgtcctaaagctgatcaacaccaagcaagtgtccgaaaggtgcttgtggccgagctaatagccctgggtatggtaaaagcttgatcanatgctacaatggataactcgcagccaagcccctaagtagagcgatctatacggggaaggttcagagacttaacgggggtgggtgtggtgtanaccatgcctaagataaagtccgtccgcctgcgaaagcttgcgtagaaaaccgtaaaaacgggagcctgcggcttaatttgactcaacac

SEQ ID NO: 53 - DNA, 448 bp, Pleurastrum paucicellulare
  source 1..448: mol_type = genomic DNA; note = SAG 463 1
gattctgtgggtgaacggtgtccgtgacactactgccccagcgggtgaaagcctacgctagtcgtccttgctcgacccaaattgctatagcaagggaacggcgagaccatcgaattgcgggaaccccctaaagcttgcaccaccaaggcatgcgggaaaccgacatgctggccagggtaatgacctcgggtacggtaaaagcgtgcaagatgcaacaatgggcaatccgcagccaagcgccctaacaggtaatgctgagggtgaaggttcagagactaagtggtggtcggtgccggagcctgtgtgccccggtgcttaagatatagtccggcactacggaaacgtagccacgagaggaaaggccagcagcgcccggagctcgtggaggctgtcggtgctccgctcaccgggantgtgactaatcgatgtgattttgtgggtggtgcat

SEQ ID NO: 54 - DNA, 443 bp, Dunaliella salina
  source 1..443: mol_type = genomic DNA
gacggaagggcaccaccaggcgttaacttagcagcaagctcagcgcctcaaagtcgaagggaaacctttggctagtatctgggtgtagatttcacctaagtgcaacactgttcaaattgcgggaaagccctaaagctttgctaaccaagctgtcctagaaatgggatggtggccaggtgaaagaccttgggtacggtaaaatcagcaaagatgcaacaatgggcaatccgcagccaagctcctacgggctgtcaaagcctatggagaaggttcagagactaaatggcagtgggcaagcatggcaatgcttgcttaagatatagtccgtcccagctgagaagctgcctatgagaggaatgccgtaaggcaggagagctaataggaagtaagtgtctttaatcaacttacttggattccacgggagcctgcggcttaatttgact

SEQ ID NO: 55 - DNA, 380 bp, Protomyces macrosporus
  source 1..380: mol_type = genomic DNA
cggaagggcaccaccaggagtaaacgtttatgtcgcagttaactctgctccgaaaagccgctcgaaagagtcatggtgtccttgaaggctagtcagttcgctggcgacatttccaaattgcgggaaactccttagagctcaagctaccgcctctgtttagtgatagacagctggcaccgggttaacgacctcgggtatggtaaaaacgtttgagattggacaatccgcagccagcttccttgagaggaagaaggttcagagactagatggatgtgggtgaagtctttggatatgaagattttgcttaagatatagtccaatccaaagcggagacgcattggttacaacaagtagaaaatgggagcctgcgcttaatttga

SEQ ID NO: 56 - DNA, 276 bp, Planktothrix sp.
  source 1..276: mol_type = genomic DNA
acggacttaaagataaattgagccttgaggcgagaaatctctcaagtgtaagctgtcaaattcagggaaacctaaatctgtaaattcagacaaggcaatcctgagccaagcctaggggtattagaaatgagggagtttccccaatctaagatcaatacctaggaaggtgcagagactcgacgggagctaccctaacgttaagccgagggtaaagagagagtccaattctcaaagccaattggtagtagcgaaagctacgggagaatgaaaatccgt

SEQ ID NO: 57 - DNA, 417 bp, Bionectria ochroleuca
  source 1..417: mol_type = genomic DNA
aattgacggaagggcaccaccaggggttaacagaccgcagtcgctctgcccctaaaagcagccgggaaactggttaggtgggaaagtccttggccggaaggccgaatactgctagtcatcgaacgatggcaacaccctcaaactgacgggaacgtcctaaagccgatcaacaccaagcaggcgtccgaaaggtgcctgtggccgagctaatagccctggagatggtaaaagcttgatcggatgctacaatggataactcgcagccaagcccctacgtgttccgacatacggggaaggttcagagacttaacgggggtgggtgtggtgtagaccatgcctaagataaagtccgtccgcctgcgaaagcttgcgaatacaaccgtaaaaacgggagcctgcggcttaatttgactcaac

SEQ ID NO: 58 - DNA, 369 bp, Prochlorothrix hollandica
  source 1..369: mol_type = genomic DNA
gtcaacccgtagaccatcaggggggcgtggcggaatggtagacgctgcggacttagataaactgggccttggtggagaaatccgcgaagtgtaagctctcaaattcagggaaacctaaggccaagggctagggcaatcctgagccaagctgagggatcaaccacccctaggggcgttgaccttcggaaggtgcagagactcgacgggagctaccctaacaggccgtctgccaaggcaggcgatcgctgagggtaaagggagagtccaattctcaaaacctctggcaccacgtcaggggcaacagtgaaagctgtgggagaatgaaaatccgttgaccgtgaggttcgtgagggttcaagtccctccgcc

SEQ ID NO: 59 - DNA, 439 bp, Dactylella huisuniana
  source 1..439: mol_type = genomic DNA
gtcgtaacaaggtttgaagtaagtatccttccgagggtgacgctcacgagaagcctttgtggccccgcaaggggtacccgccgcgactgtaaagaatgaaagcaggtattaaatcgcaagtcagctctgctggccacactttcgaattgcggggacaccctaaagcctgcctctaccaaccgatcggggaaacctgaacggggcctgtgctaacgacacagggtacggtaacaacgaggaggatgcgaagaaatgggcgatccgcagccaacccctaaggcctgaaaaaggctacgggagaggttcacagactaagtggaagtgggctggaggtgttgaaaacctccggcttaagatatagtcgggccctgcgcgagagcgcgggggagtcacaactggactgaaccgttccgtaggtgaacctgcggaaggatcatta

SEQ ID NO: 60 - DNA, 434 bp, Graphium putredinis
  source 1..434: mol_type = genomic DNA
aaattgacggaagggcaccaccaggggttaacgtctaactgttaagccacagaagaaatctgctcctgaaagccgcccgaaagggtgaggtggtgtccggagcgtgcgagctcgaatttaaaggctagtcctccaatgcggagggcgacatcctcaaattgcggggaactcctaaagctttaactccaaagccggttcgcgaaagccgccggtggccaggttaacagcctcgggtactggaaaaacgtggaagatgcgacaatggacaatccgcagccaagcccctaaaggctccccgtcgagggacgcccacggggaaggttcagagacttaacggggatgggtattcgccgagaggcgcctgcttaagataaagtccggccctgcgcgagagcgcgggggtagcagggaactgcggcttaatttgactcaac

SEQ ID NO: 61 - DNA, 437 bp, Capronia pilosella
  source 1..437: mol_type = genomic DNA
ggcgaaagactaatcgaaccattaagaagccactcggtggttgagtgtaggcccgaaagggtccgcaagtgccggatcctggcgcgacactgtcaaattgcgggaacaccctaaaactccagggaccgcctggcccccgaaagtgggccgaggcaccaggttaacgacctcgggcacggtaatactcctggagatgtaacaatgggcaatctgcagccaagtcctacggggtcctccggggcctacggatgcagttcacagactagatggcagtgggctcggagagcttaagatatagtcggccccctggggagacccaggcgtgtcgctaaactcgaggtgaaggattccgcccccggcggccacctcgagcctgaacgacgcgggcctgtgttaaacaggtcggataagcgctggtagctggttcctgccgaagt

SEQ ID NO: 62 - DNA, 426 bp, Gaeumannomyces graminis
  source 1..426: mol_type = genomic DNA; note = var. avenae
gggagtaactatgactcaagaattggcgaacgtgagagggtcatgaaagcggcgggaaaccggcgctagtgggcagccctgcctgcgacattgtcaaattgcgggaacctcctaaagcctcgcccaccgcggcccgccgaaaggctgggtgcagcaccaggttaacgacctcggggacggtaatagcggcgaggatatgtggacaacctgcagccaagtcctaagttttggggtccgacgcgatgtcaagcgccggacctgggccaaaggcgaccgaaatagggatgcagttcaacgactagacggcaatgggtccccggggtgacggccacggcgcttcgccgtcgtcccatacaaggggcttaagatatagtcttgtggtccgcttaacggcgtgtctgccacgtaaaagctcttaaggtagcc

SEQ ID NO: 63 - DNA, 308 bp, Aspergillus pseudoglaucus
  source 1..308: mol_type = genomic DNA
cgctagggatttataactgtgagtcctccaatattataaaatgttggtaatatattgggtaaatttcaaagacaacttttctccacgtcaggatatagtgtatttgaagcgaaacttattttagcagtgaaaaagcaaataaggacgttcaacgactaaaaggtgagtattgctaacaataatccttttttttaatgcccaacatctttattaactagttaataaagatgatgaaatagtctgaaccattttgagaaaagtggaaataaaagaaaatcttttatgataacataaattgaacaggctaa

SEQ ID NO: 64 - DNA, 438 bp, Arthrobotrys cladodes
  source 1..438: mol_type = genomic DNA
gtcgtaacaaggtttgaagtttgttcccttccacaggtgacgctcacgagaagcctttgtgaccccgcaaggggtacccgccgcgactgtaaagaatggaatgtgggtattaaatcgcaagtcagccgccgctggcaacactttcgaattgcgggaacaccctaaagcccacctctaccaaccagcctgagaaatccagctggggcccgtgttaacagcacgggaaatggtcataatgaggtgggataggtcagcctttccggttggcatcatggacaatccgcagccaagtccctaaggtccagctggggctacgggaaaggttcacagactaggtggaagtgggttggttcagcaccaacttaagatatagtcgggctctgcgcgaaagcggaggggagtcactgttccgtaggtgaacctgcggaaggatcatta

SEQ ID NO: 65 - DNA, 400 bp, Geosmithia viridis
  source 1..400: mol_type = genomic DNA
gaaagttaggggatcgaagacgatttaagaagggtccgcgccccccccggtgggggccggctcggtatttggtcgttaaaagtaagctcaaagtgtcttgctagtcgccgcgaccgcgggggcaacaccgtcaaactgcgggaacgtcctaaagcctgaactaccaagccgacgccgaaaggcgccggtggccggggtaatgacccagggtacggtaacaacgttcaggatgcgccaatggataacctgcaaccaagcgctaccgcctattggccatgcgtgcagttcacagactagatgtgggtggggggttgtaccccttaagatatagtcgaagcccagtgtgaaagcctgggattagttgcagataccgtcgtagtcttaaccataaactatgccg

SEQ ID NO: 66 - DNA, 254 bp, Thiomargarita namibiensis
  source 1..254: mol_type = genomic DNA
aggattagatactacactaagtgtcccccagactggtgacagtctggtgtgcatccagctatatcggtgaaaccccattggggtaataccgagggaagctatattatatatatattaataaatagccccgtagagactatgtaggtaaggagatagaagatgataaaatcaaaatcatcattaaagttatagaattatcagagaatgatatagtccaagccttatggtaacatgagggcacttgaccctggtag

SEQ ID NO: 67 - DNA, 331 bp, Allomyces macrogynus
  source 1..331: mol_type = genomic DNA; note = Mitochondrion
ctgcgttagttgaatttgtttaacaagttccggagaatagtaatattctcatgaaaatcgggtgaattgtcaaggagacctcctacaatggagatgacttgcagcgaagcttatatcagtgcttaatagatataagaacgttcaacgactaatcggtgagtagcactaacaataaaccggacactaggtggtgagtacaaccaacaataatccacccacgagcgcccgacaaccataattatggttgatgacatagtctgaacaggcaggtgactgtctgaagagaaatttaaacgatttctcgataacataattgttgtttggggtggat

SEQ ID NO: 68 - DNA, 229 bp, Trimorphomyces papilionaceus
  source 1..229: mol_type = genomic DNA; note = Mitochondrion
tacctcgactacggtacttttgtgccctgctttagcagtaaccgggttaattttgagaacatctaataagacaattcaaagcctatatataactgtataagttgaacgactattaataataattaactagaaaacctggctcctacttggagatgaaatagtctaaacattagtgtgaactaatgaaaccaataaaaggtgataatatgtattgacgatcagttaatgg

SEQ ID NO: 69 - DNA, 400 bp, Genicularia spirotaenia
  source 1..400: mol_type = genomic DNA
aagtcgtaacaaggttttgccttgagcagacgcttgcggaagccttagcagcccacaagggttcccctcgcgactatatttaaaggaaggggattgaatgctagtgcctgagctgccaacttgggtgcgacatcgccaaattgcggagaactccacatttgcctcgattaccaagcggaccgccgaaaggaccccgtggccgggctaattgctttgggtatggtcaaaacatcgaggaaaaggatgatccgcagcgaaaccctcttgttgtacaaaagtgggtggcgttcacagactaaacggcgatgggttccagaaattcgaaacctgggcttaagatgtagtcggtttgtcttcgaaagtggcagattgcagagtttcaccgttccgtaggtgaacc

SEQ ID NO: 70 - DNA, 276 bp, Oscillatoria williamsii
  source 1..276: mol_type = genomic DNA
gtagctcgtcgggctcaatacgcaagttaactaaacgcttaatagttagcattgcgatgggctgcatactaaggaaactggtatgcgatagcctctcaaattcggggaagccggtagcgcggtaatcccgagccaagctctgaacaagagaaggtgtagagactcgatgggaggcaccctaacagttaaagctgagggtgaagggagagtccagaccacaaactggaaagataccgggcagcgaaagctgtagatggtacgcataacccgaaggtc

SEQ ID NO: 71 - DNA, 248 bp, Anabaena sp.
  source 1..248: mol_type = genomic DNA
aaataattgagccttagagaagaaattctttaagtggatgctctcaaactcagggaaacctaaatctagctatagacaaggcaatcctgagccaagctgaagtagtaattagtaagttaacaacagataacttacagctaatcggaaggtgcagagactcgacgggagctaccctaacgtcaagacgagggtaaagagagagtccaattctcaaagccaataggcagtagcgaaacgtgcggagaatg

SEQ ID NO: 72 - DNA, 216 bp, Oscillatoria sp.
  source 1..216: mol_type = genomic DNA
agaaaactgagccttattggagaaatccattaagtgaccgctctcaaattcagggaaacctaactctggtaacagacaaggcaatcctgagccaagccgaaatttcggaaggtgcagagactcgacgggagctaccctaacgtaaagccgagggtaaagggagagtccaattctcaaaaccagaattctggcagcagcgaaagttgcgggagaatg

SEQ ID NO: 73 - DNA, 255 bp, Microcystis aeruginosa
  source 1..255: mol_type = genomic DNA
acgcaacggacttagaccaaacttgagtgctcgcagggaaacctgcgatgcagaactgctcaaattcggggaaacctcttaactggcaatcccgagccaagccttggcgcgaaagtgcctcggaaggtgtagagactagacgggcagcacctaaaccccgccagcggggcacggtgaagggatagtccagaccacaaacattcgggctaccgagtggcggtgaaaaccgtagcaggtaagaaaatccgtcgggct

SEQ ID NO: 74 - DNA, 327 bp, Fischerella ambigua
  source 1..327: mol_type = genomic DNA
cggtcaacggattttcattctcccgcagttttcactactgcctagtagactagctacttgtggctttgagaattggactctccctttaccctcgacttaacgttagggtagctcccgtcgggtctctgcaccttccaaataagttgtttgttatttatcgcttgtcttttgttattcaacaattgacgaatgacaaatcacaaatgactaatttggcttggctcaggattgccttgtctgttgccagatttaggtttccctgaatttgagagcattcacttaaagagtttcttctttaaggctcaattacttaagtccgtagcgtct

SEQ ID NO: 75 - DNA, 295 bp, Synechococcus sp.
  source 1..295: mol_type = genomic DNA
tcacgacctttacagtcagcggattttcattctctctgcaactttcgctgccatcaacaatcgcgatgttgagaattggactatccctttaccctcggctgaatccgttagggtagctcccgtctagtctctgcaccttctaaagctgttagttgtttagctttagcttggctcaggattgccatgactttaatgtttaaagttttaggtttccctgagtttgagagctgccactcgatccctttcgcgatcgaggcccagttttctaagtccgctgcgtctaccgttccgccac

SEQ ID NO: 76 - DNA, 287 bp, Synechococcus sp.
  source 1..287: mol_type = genomic DNA; strain = PCC6301
cgacctttacagtcagcggattttcattctctctgcaactttcgctgccatcaacaatcgcgatgttgagaattggactatccctttaccctcggctgaatccgttagggtagctcccgtctagtctctgcaccttctaaagctgttagttgtttagctttagcttggctcaggattgccatgactttaatgtttaaagttttaggtttccctgagtttgagagctgccactcgatccctttcgcgatcgaggcccagttttctaagtccgctgcgtctaccgttcc

SEQ ID NO: 77 - DNA, 406 bp, Gentiana brachyphylla
  source 1..406: mol_type = genomic DNA; note = Chloroplast
agacgctacggacttaattggattgagccttggtatggaaacctactaagtgataattttcaaattcagagaaaccctggaattaataaaaagggcaatcctgagccaaatcctagttttcgaaaaagcaaaaagaaaggcttagaaagaaatgaaagaaataaaggataggtgcagagactcaacggaagctgttctaacaaatggagttgattgcgttggtaaagaaacctttctaccaaaaattccaaaaggatgaggaaaaggggtatatacagactgaatcaaatgattcacccacaacctgcagatcttttcacgaacggattaatcagacgagaataaagagagagtcccattctgcatgtcaatgccgacaacaatgaaatttatagtaagaggaaaa

SEQ ID NO: 78 - DNA, 355 bp, Mycosphaerella juvenis
  source 1..355: mol_type = genomic DNA
agggcaagtctggtggacgtccagtcccaatgctggcgccagaagcagtgaggccccaactcactagtcgatgcacccaccaacagagagggcgggtgccggcaagacgacctggtacgagggaccccttaacggccgcctataggtggggccgaaggcaatctcgtggcgacctccctcacacggaggcgtcgtaacgcgcggaaaggcgtcggtcgcgataagcggcttaaggaacgtgctagacccgagggaaacctcgctccttctgatcagccccctctcggcgaagcagtggaggcgcccggcgaacgggtgtggacgagtcaatgaaaatcggccagcagccgcggta

SEQ ID NO: 79 - DNA, 403 bp, Barrmaelia oxyacanthae
  source 1..403: mol_type = genomic DNA
agggcaagtctggtgaactgaactcagcttccagctggcgccagagatagtggttaaaatcactagtcgagcatcctctcccacggtgaggacgccggcaaggcaacctggattcggggaaggctaaggggcttacgggccctatgccaatcccgagcagaaattacttctgtgtagagcgcggaaaggtgccggtcgtgagacctaccctggaacagcctaccctgtagcaagttaccctgtaacgagttaccctgtaggccttctcggttggggctcgcggctcgagggacgtgccaaccacctacgaaagtaggttctagtatcccgagcgtagagctaggggtatggagccctgtattttagggtcgtcagtcactgatagtggccagcagccgcggta

SEQ ID NO: 80 - DNA, 281 bp, Leotiomycetes sp.
  source 1..281: mol_type = genomic DNA
ggagcctgcggcttaatttgactcaacacggggaaactcaccaggttaactacagttacgtctgggcctggaatagttatttgacaactagtacacgcctttacttgtgtgggagaagtctcccaattttggacacaaggcctttcaggagggtttggcggtgcgacactacctggtacaggggacgcctcgctggagtgatccggcaggccgactctgtggcgagctcgagtaacgtcgagccgtcgcaacggcgggaaaggagtgggttcggtggatct

SEQ ID NO: 81 - DNA, 365 bp, Tilletiopsis flava
  source 1..365: mol_type = genomic DNA
gaggcaataacaggttaacctatcacaggcctgtaacagctgcaaggcagctagtccagcgtcgacgtctgcgtttgaggcagatggggtcttttggaccttggcgcgggcgacacaacctggtacaggaaacgccccggcagcaatgccaggccgatctgttggcgagctctgcgaggagccgtcgcaacgtgcggaaaggtgtgggtctttctctatgaaaggcttaaggtacgtactaatccccggggaaacccgggcctctgcgagaagagcccaaactctatgcgcagggggaagcttgcggaatgcaaagctttggcgatgatcctttgtgacatcgataaatgctgtgatgcccttag

SEQ ID NO: 82 - DNA, 328 bp, Papulosa amerospora
  source 1..328: mol_type = genomic DNA
cgatggaagttcgaggcaataacaggtcaacgcatcgcaggcctgtaatagtttaaataacgctagtccgctggatttacagcgggcgacactaccttgtacaggggacgcagtgctcgtcttgggagtctgccaatcctgtggcgagccctttgggccgtcgcaacgcgcgcagggagtgggctggggcttattggctccggcttaagatacgtgctaagctactgggtgaaaaccaggcttcgcgcttaaaaacgcggaggatttcgtgacgaacttagaaatagactgctgtgatgcccttagatgttctgggccgcacgcgcgt

SEQ ID NO: 83 - DNA, 415 bp, Chlamydomonas moewusii
  source 1..415: mol_type = genomic DNA; note = Chloroplast
cagcagccgcggtctggcgtaactaatagaagttgcaatgccgccatagcgagtgatcgttatgtaaaaaaaccctgctcataacggtgaagccttagtaatttatctttttagggtattttacggtaataccgtgggaagtttatcaacaaaaggctgcccgcggcaaagccgcgggcacaaaaaaagcttttttaaaacgtgttgatataaccccgtaacgacttttcttgaaagagaaagaggtgctttattagcgcctaacacgcaggcacttacgagtcctaaatgcagcccgcggaaaccgcgggcggctatggtaataatctagcccgcggctttgccgcgggcgatgcctaaagtctaaagtaagttgaaggtatagtctgagcctctggaaacaggggaataatcg

SEQ ID NO: 84 - DNA, 383 bp, Chlamydomonas callosa
  source 1..383: mol_type = genomic DNA
gcccattaaagtggtacgtgagctgggttcaaaacgtaaataacactgcgtgtagttgtaaaaatacaactaaaagatttggcttatatcggcgaaaccttactatctttttaactgtacgatagatggcaatgccgagggaagttcttattctaaattaatagattaagacacctgtagagactttataagtttataaaacttataagatacttcctacaaaggaaagtataatacgccaacccctactttttacctgagcttataaacaagcgaaaggctaaagcggggaggagatatagtccatgctcttatgaaaataggagggaaggtttaacaatatttgttaaactgcatgcgtgagacagttcggtccatatccg

SEQ ID NO: 85 - DNA, 372 bp, Amoebidium parasiticum
  source 1..372: mol_type = genomic DNA
ggaaagtagacgcgttccggtgaagagtgttctacccttacgacaaaccgctcgaatgggccttgtgagagacaactctcacattgcagttggctatatgctagaaactaccgttgtattcataaatacaaaatgttatctactcttattgtatatgggagtgacaatgatggacacgaggtacaattagcaggaaaccaaaggttacaagggtaacttagtaggatcttcagagactacacgccaaccatcttgaattagcaagatgaagacatagtccatagtggattagtaaagatactagtattaatattaatctactagcgtcaagctgagaaagggttagttcgatgaagacaatcagtgttaagg

SEQ ID NO: 86 - DNA, 312 bp, Chara vulgaris
  source 1..312: mol_type = genomic DNA
agcgaaattcctcgacaaaggggtattttctgaacagcatcaaagacaaaatctggctatatgctggaacctccgagtgacgcaagcaaaatgcgtccggacaatcagcagggaaggaagtcatacatgtgttgatacatgcgctgcgcccctcaacgactacacgccagacatccctgcttgggatgatgatatagtctgaacttcgtggcgacacgaaggaggaaggcctcggttgaaatgatgttcgaagtttatttcaattaacacaattgtgtcgcataagtagcgacctgcacgaatggtgtaacg

SEQ ID NO: 87 - DNA, 227 bp, Chlamydomonas moewusii
  source 1..227: mol_type = genomic DNA; note = Mitochondrion
cgacgcgcgtgttcggcaccttgaggcgctacgcaagcgtagcgccgattcatcggggtatttttatagtgatataaaaaaaagaaaaaggctaaaagctggaaaatctataatatgataatcagctggaacaaaagtatgtaataagtaatagttattatatataaaagattcttcaacgattaaaagcctagctcttttaataaagagtatgatgtagtctgatc

SEQ ID NO: 88 - DNA, 267 bp, Pyricularia higginsii
  source 1..267: mol_type = genomic DNA
atttagccgaggctaaaaaaatattctgatttggtctggttatatgtcgcaaattcctggaaaatctttataaggtaatcagcaggaaaacttaattaatttagtgattaaactcttcatcgactaaacgtgacaatttaatttttttattaaataagagatagtcaaaatttcgatgtgaatcgaataatatttattataagagcaccaggttcccagagcgctaggcgattattataatttatattagccctcccttggaatcag

SEQ ID NO: 89 - DNA, 414 bp, Stigeoclonium helveticum
  source 1..414: mol_type = genomic DNA
agtgaacggcggccgtaactatgacggtcaactttagggccgtctttaaagataactttttgctggaacctccataaaaagagcaatcagcaagagattatattatttgttctaataatttcaacatctgtataaatttccgtctacattaaatggcttactttacgaatgtcttcctttatgggatgccatgagaatacaaaaaggccaaaaaaatcaaagttttgaaactcttgaagacgcgcaaaattatgtacgagattttgttgcgaacaaataaaaatagtttcttcagagactactggttatccgattttaaatattttagtatttaagattgaagatatagtccgatattatatgaatgtgtaataaataccttgcctaaggtagcgaaattctttgtcgagtaag

SEQ ID NO: 90 - DNA, 240 bp, Neochloris aquatica
  source 1..240: mol_type = genomic DNA
ggcagctgtaactatgacagttctaaggttaagaaatgctagctttagtaaaatcaaactatttgctgggaacccgtaataaaggaaatcagcaggtaactctataataatttatttatagagcacctcagagactatacgtttgacgttttgatgaaaacatcaactttaagatagagtccagcgttttttgaaagaataacgattatctgagcgaaattcattgtcgagtaagtttcg

SEQ ID NO: 91 - DNA, 406 bp, Pedinomonas tuberculata
  source 1..406: mol_type = genomic DNA
caggctgatcttccccaagagttcacatcgacgggaaggtttggcacctcaactggggtctatcaatgtgaattgataatgcacaagttagctatatgctggaaaatccggaggatccaaaaacccaccctaattttgttaggtcggaaaagttttttggttcggacaatcagcagggaagaccatgtgcccctcaacgactacacgctgacctacttaaaaaaaaagtagatgatatagtctgaacttcaaggcgacttgaaggtattcatttttctgtgaacgaactgcaactagcagcacgcgaaaaggaacacgttttcgattgcattcgaaaattaacgtctttgcgatgtcggctcatcgcaacctggggcggtagtacgtcccaagggttgggctgttc

SEQ ID NO: 92 - DNA, 428 bp, Chlamydomonas starrii
  source 1..428: mol_type = genomic DNA
caggctgatcttccccaagagttcacatcgacgggaaggtttggcacctcgacatagggggtcgctttcacgcaagtgaaggctgtaaattagctgtatgctggaacttacctattaggtaaaatcagcaggaagtgaagcacacgtgtttagctcctcaacgactacatgctaacactcttgtccttgcgtcctgtgccgcgccgcgccttcttgtccttcgccgaaggcgcggacgcgaaggcgaaggcaaggacgcgaaggggaaaggacgcaagagcagagtgatgatatagtctgaactacgctttaacggcatagaccaacttagttggtttctttctagataacctaggagggataacatatttgcgatgtcggctcatcacatcctcggtctgtagtaggtccgaagggttgggctgttc

SEQ ID NO: 93 - DNA, 414 bp, Chara sp.
  source 1..414: mol_type = genomic DNA; note = CL 2002 1
gatcttccccaagagttcacatcgacgggaaggtttggcacctcaaatcggggcaatagtccagtaatgggaaaataaagtagagaatttggctatatgctgggacatccgtagtttctaatctaaaaaaataagtgacaatagcgtaggaaatgaattgaatgaattcggattatcagcagggaagtttcttcaagatacccctcaacgactacacgccatgtatcctttgcttttcaaggataatgatagagtctgagcttttaggtaactaaaaggccactattgagtgttagtggcgttaaaccatacatgtcgaaattccccggaaaattgataatgtttaattaacacattgcgatgtcggctcttcgccacctggagcggtagtacgttccaagggttgggctgttc

SEQ ID NO: 94 - DNA, 434 bp, Chara corallina
  source 1..434: mol_type = genomic DNA
gatcttccccaagagttcacatcgacgggaaggtttggcacctcaaatcggggcaatcgtccagtaatgggaaaataaagtagagaatttggctatatgctgggacatccgtagtttctaatcaaaaaaatcagtaacaatagcgtaggaaatgaattgaatgaattcggattatcagcagggaagtttcttcaagatacccctcaacgactacacgccatgtatcctttgcttttcaaggataatgatagagtctgagcttttaggtaactaaaaggccactattgagtgtgagtggcgttaaaccatacatgtataaattacccggactaaaaatccataaaaaaatcaaattgagaatgtttaattaacacattgcgatgtcggctcttcgccacctggagcggtagtacgttccaagggttgggctgttc

SEQ ID NO: 95 - DNA, 428 bp, Selenastrum capricornutum
  source 1..428: mol_type = genomic DNA
cctcttacacggataaccgtataatacatgcggaagaagtactcgcgttcctagtagccttaaaaggctgcgacattgcaattgcctgaaccctgttaatctcaccagtaccgctgctgtcccgaaagggaccacgcagcaccttggcaacgcccgagggtgtggtaaaaacctgaagagtaggggcgatgggcagccaagtcctgttggtagccaatacctacggatgcagttcacagactaaatggtaatgggttcttgagtttgactacacaagagcttaagatatagtcggtgggtgccaaaaggcagaccgcaggaggacaacaaccacacgttgtctgagagcctgtggtggacagtaatagttgggtgcaaccaaccggtctgaccgtctgctcccggaacaaacgagtaattctagagct

SEQ ID NO: 96 - DNA, 415 bp, Chlorella trebouxioides
  source 1..415: mol_type = genomic DNA
tactcggataaccgtaaggattnttgcgggagggaancgaaagcctgccctagtggctcgaaaagcgagctgcgacactgtcgaattgcccggacgtcctgctacgctggagggaccgtctgggtcgggagaccggcctggacactcgtggcgaaagcctcgaggtacggtaacactcctcccgctaggggctatgggcagccaagctctaagtcgctctcgcagcgatacgagtgcagtccacagactagatggcagtgggtatcgctcatcgaagcgatgcttaagacatagtcggtccccaccgagaggtggttcgacggaggatcgaggcgaccgcctcgtgagagccgccgaggtcgggcgaggtctagctagacgacccgccgacggagcacacgagtaattctagagc

SEQ ID NO: 97 - DNA, 406 bp, Delitschia didyma
  source 1..406: mol_type = genomic DNA
gaacgaaatttaggggatcgaaaacaattttacagttaaacgtgacagtttctggtcgtcgaaagtgcgtaagcactaatctgccccccccgggggcgggcgacatcatcaaattgcggggacccctgggaaccccagctaccacacacggttgaaaggcactgtgccaacttcggagagagaccgaantgaagtaaaaacgctgggtcaggatctattcctacaggaaatccgcagccgacgccctaaaccccctcccgggggcatgggtgcacttcacagactaaattgtgatgggtarggggcaytscctcctgcttaagatttattcgggcccggtgcgaaarcgstngnaacaatcttaanaatycgncagataccgtcgtagtcttaaccgtaaactatg

SEQ ID NO: 98 - DNA, 445 bp, Coelastrella multistriata
  source 1..445: mol_type = genomic DNA
gaattgacggaagggcaccaccaggcgttaataagagctgctcagctttagcgccagtggcttggaaacaagctgctagtcacactcctatttgtgagtcagagacttgctatagcagtgtggcaacatcctcaaattgctgggaagccctaaagctgctacataccaaagttggttggaaacaactgactggccaggttaacaacctcgggtatggtgacaactgtagcagatgaagcatgctcacatgctgaagtgggttatcagcagccaagtcctaaacgtgctcggtgtgttcaactctaccgcaaggtagcttattccaagacacaccaagcatgcatggatgcagttcacagactaaatgggggtgggccctcgaagcttaagatataggagcctgcggcttaatttgactcaacacgggaaaacttaccaggtccag

SEQ ID NO: 99 - DNA, 399 bp, Naohidea sebacea
  source 1..399: mol_type = genomic DNA
gtctaggttcgggggggagtatggtcaagcgttgcggccatgaagagtgacaaggtaacttgttgctagtctatttaataggcaacaccgtgcaaattgcggcaacacccttaagcttgttaacaccaagcacggtaccgaaaggtacgtgtggccagatgtagtgatctgggtatggttatagtttatcangtttgggcaagcacgcagccaagttcttactcgttagcgagagaatgcagttcacagactaaatgccggtgggtaggaaaaagcaatttcattgcaaaagtgttcttatcaaatcctacttaagatatagtcgggccacgtcgaaagatagtggattagcctgcgcaaggctgaaacttaaaggaattgacggaagggcaccaccag

SEQ ID NO: 100 - DNA, 396 bp, Sclerotinia trifoliorum
  source 1..396: mol_type = genomic DNA
gaaacttaaagaaattgacggaaaggcaccaccaggcgttaactgcagtaactctgcgcctaaaagcagctcgtaagagttggtggtagccttcaaatatgctagttggaaatcagctataccttcaaactgcggggaactccttaaaaactcaactactaaacctcaattgaaagattgtggtggccagctgattctgggtagagtaataatgttgagaatttggacaatccgcatccaaccctctaaggttttaaactacgaggaaggttcagagactaaatgtaggtaggtagcgtcgctacttaagatatagtccgcctcgagattaacgtctcaagaataacaatgggagcctgcggcttaatttgactcaacacggggaaactcaccagg

SEQ ID NO: 101 - DNA, 420 bp, Pseudallescheria boydii
  source 1..420: mol_type = genomic DNA
ggaagggcaccaccaggggttaacgtctaactgttaagccacagaagaaatctgctcctgaaagccgcccgaaagggtgaggtggtgtccggagcgtgcgagctcgaatttaaaggctagtcctccaatgcggagggcgacatcctcaaattgnggggaactcctaaagctttcactccaaagccggttcgcgaaagccgccggtggccaggttaacagcctcgggtactggaaaaacgtggaagatgcgacaatggacaatccgnagccaagcccctaaaggntccccgtcgagggacgcccacggggaaggttcagagacttaacggggatgggtatgcgccgagaggcgcctgnttaagataaagtccggccctgcncgagagcgcgggggtagcagggaacctncggcttaatttg

SEQ ID NO: 102 - DNA, 350 bp, Sporobolomyces sasicola
  source 1..350: mol_type = genomic DNA
acttaaaggaattgacggaagggcaccaccaggagttaacgcaagacaaagctctgctcttcaaggcgaaccagaaatggtttgtggtgtcccttgtaacattcgccagtactctacgcgagggcgacactttcaaattgcggggaactcctgagagccctcagctaccaagtcgaagcgctgaaaagcctttgatggccagattaaaaatcttgggtacggtaacaacgttgagggattggatgatccgcagccgcctccccttcaatggggcgcggttcagagactaaacggaagtgggtccactctaaggatctaagatatagtccatggcttgaggaaactcaaag

SEQ ID NO: 103 - DNA, 448 bp, Nigrosabulum globosum
  source 1..448: mol_type = genomic DNA
ggctgaaacttaaagaaattgacggaagggcaccaccaggggtaaaatccgcagaagctctgcccctaagagcagccgggagaccggtcggtgggagaaaacagcccgggcgccgcagagcgcgaaccaactgctagtcggcccccccgggggtccggcgacatcctcaaattgcggggaatccctaaagccgcgtgctaccaagtgggccgccgaaaggccgcccgtggccgggtctaacaaccccgggtacgttacaacgcaccggatgacaacaatgggtgacctcgcagccaagctcctacacggcttgccgcgcaggaggaaggtccagagacttgacggggatgggtcccgccgccagccggcgtggcctaagataaagtccgtctgcgcgcgaaagcgctgcagagccctaaacgggacgtgcggcttaatttgactcaac

SEQ ID NO: 104 - DNA, 440 bp, Beauveria bassiana
  source 1..440: mol_type = genomic DNA
attgacggaagggcaccaccaggggtaaaccgcttcatgcagcccgcagtagctctgctcccaaaagcagcccgaaagggtcaagtggtgttcctgtccgcgctccggcgcggcgaaccaaagtgctagtctctccggaggcgacaccctcaaattgcgggaaacttctaaaaccagtgtcaccaagccggcgtcgaaaaggcgtcggtggccggggcaatgacctagggtacggtaaaagcacactggatatatggacaatccgcagccaagcctcgtcacggggaaggttcagagactaaatgggggtgggtagtgtgctggaggattcgtcctccgagcgctcgctcacacactgcttaagatatagtccgcccgcgctggaaacagcgcggattggcaagctcaaataaacgggagcctgcggcttaatttgactc

SEQ ID NO: 105 - DNA, 447 bp, Mycoarachis inversa
  source 1..447: mol_type = genomic DNA
ggctgaaacttaaagaaattgacggaagggcaccaccaggggtaaaacgccgcaagctctgctccggaaagcagccaggagactggtcggtggtgccttcagaacacaagtgctagtcaccctccgcggtggcgacaccctcaaattgcgggaaatccctaaagccgcgtgctaccaaccggaccgccgaaaggcggccggggctgggctaactgccacaggtatggtcacaacgcaccggatgagcggccgagaggccgcgaagtgggtgatccgcagccaagctcctacgtagaggtcccctctatacggagaaggtccagagacttgacgggggtgggtgcggcagcggccgcgccgaagataaagtccgtccggcgtgcgaaagcgccccggatccctaaacgggagcctgcggcttaatttgactcaacacggggaatctca

SEQ ID NO: 106 - DNA, 360 bp, unidentified
  source 1..360: mol_type = genomic DNA; note = uncultured soil fungus
ggaagggcaccaccaggagttcgccgaactgcgatttattagcagactctgctcctgaaagccgcccgtagaagggtcggtggtcgtccaggcttcgagccgaacaagtgctagtcacgcgagtggcaacatcgcccaactgcgggaacgtcctaaagctctacctaccaacccgccgtcgaaaggcgtcgggggcccggttaatgcccggggtacggtcacaacgggagagatatacaatggataatccgcaaccaagcccctacagcaatctgccacggggaaggtccagagactaaacggcggtgggctcacggggagcttaagatatantccgtccggggatcagcgtcccacgga

SEQ ID NO: 107 - DNA, 424 bp, Kirschsteiniothelia aethiops
  source 1..424: mol_type = genomic DNA
gaaattgacggaagggcaccaccaggcgttgtggcagactctgcgccgagatgcggccccccaggcggggggtgggtggtgccgtccttaaatgccagtctccgacgagcctcatcgtcgggggcgacatgccgaaattgcggggacatcccaaggctccgaccaccaggtcgccccccgaaaggcgggcgacgaccgggttctccccgggtagggtgggcacggtagagctggggatcactcgcagccgagcgccttccgttccatcggggccccgagggcccgggtgggaccacggcgccgggtccagagactagacaggcatgggtgggccccgttgcggaggggcccgcttaagatatagtccacgagcatccgaggatgcctccatcagggagcctgcggcttaatttgactcaacacg

SEQ ID NO: 108 - DNA, 439 bp, Protomyces lactucae-debilis
  source 1..439: mol_type = genomic DNA
tatggtcgcaaggctgaaacttaaagaaattgacggaagggcaccaccaggagtaaacgtttatgtcgcagttaactctgctccgaaaagccactcgaaggagtgtggtgtccttagaggctagtcagtacgctggcaacacaatcaaattgcgggaaactccttagagctcaagctaccgctgctgcccagagatgggttgtggcaccaggttaacgacctcgggtatggtaacaacgcttgagattggacaatccgcagccaagctccctcacgggaagaaggttcagagactagatggttgtgggttgaacacacgtttattgaaacatgttcggcttaagatatagtccaatccagtgtggaaacgcactggtctaaaagatagaaaatgggagcctgcggcttaatttgactaacacggggaaactcaccaggt

SEQ ID NO: 109 - DNA, 360 bp, Bensingtonia ciliata
  source 1..360: mol_type = genomic DNA
accaggagttaacaaacaggctctgctccaaacagtagctcgaatagagtacggtggtcgctgtcgtttaaatactagtctgtttataacaggcaagatcttcaaattgcggggaagtactggggctctaatctaccaagtcattgtctgaaaaggcatgatggcctgaggtaataactcaggatatggttacaacgattagagatgttacaatgtatgattcgcagcccaatccctcaatatgggatcaggttcagagactaaacggagatcgatctaggcattgaactaggtttaagatatagtccattattctgcttaacagcaattgaatgtattaattgggagcctgcggcttaa

SEQ ID NO: 110 - DNA, 424 bp, Pyxidiophora arvernensis
  source 1..424: mol_type = genomic DNA
acaccaggggtaaacttcgaactgtagccgcagtcgctctgctcctaaaagcagccgggaaaccgtcaggtggtaagctccttgcccaggtgggcgaagtattgctagtcgtcgtaagggcaacaccctcaaattgacgggaagtccctaaagccagctgagaccaagcgagtatccgaaaggtgctcgtggccgagctaatagccctgggtatggtaaaacagcagctggatattacaatgggtaatccgcagccaagctcctaaacagatttatactgcacggagaaggttcagagacttgacgggggtgggtgcgtaattggtttttattgaactgcttacacgtgcttaagataaagtccgttgctgctgtaagcaggacaggtcttggtaacctttgaccattaaagggagtgcggctt

SEQ ID NO: 111 - DNA, 448 bp, Geomyces pannorum
  source 1..448: mol_type = genomic DNA; note = pannorum
cttaaagaaattgacggaagggcaccaccaggagtaaacttagtttcgtcgcagcaaactctgctccagaaagcagcccgaaagggttagtggtgtcccaggcctcgggccgaattaaatgctagtctgccacggcaggcgacaccctcaaactgcggggaactcctaaagcttctaggtaccaagccggcgtgcgaaagctgccggcggccagggtaattgcctcgggtaccgccaaaaccctagaagatgctacaatggacaatccgcattcaagcccctaagggaccacggtcctacggggaaggttcagagacttgatgggggtgggtttgagaatactcaggcttaaggtaaagtccggccgctggagaaatccatgcggattggtaaataaaacgggagctgcggcttaatttgactcaacacgggaaactcaccaggtcca

SEQ ID NO: 112 - DNA, 448 bp, Penicilliopsis clavariiformis
  source 1..448: mol_type = genomic DNA
tcgcaaggctgaaacttaaagaaattgacggaagggcaccacaaggcgtaaacttttaccgctagccgcagtaactctgcgccgaacagcagcccgaaagggtgatgtggtgaccctctaaacagtaaatgctagtccagcacggctgggcgacacgctcaaattgcgggaaaccccttagagcctctagctaccaagccgccggtcgaaagacggcggtggccaggttaattccctcgggtacggtgataacgctagaggattgggcaatccgcagccaagcccctacgttccgcagggaatacggggaaggttcagagactaaatgggtgtgggttgggtcctccgggggaggacaccagcttaagatatagtccggcgccaggggaaaccatggcggttcaggtatacctgtcttaacgggagcctgcggcttaatttgactcaa

SEQ ID NO: 113 - DNA, 448 bp, Dactylella cylindrospora
  source 1..448: mol_type = genomic DNA
cttaaaggaattgacggaagggcaccaccagatgtaaaccgtagttaactctgcatctgaaagccgtctcgggagtagcatcctgaaggttgaatctggccctgttcgcaatggctagtccaagctttttcagctgggcaacaccctcaaattgcgggaaactcctaaagctgctgaacaccaaacctgaccggtaacggttgggtggccgccctgaaagggtgcgggtatggtaacagagtcagcagattgctcgccagcgggccagcaaatggacgatccgcagccaagcgaccggttcaaacccaaccggttgaaggttcagagactagatgggggtgggttttcaaaaacttgaaggcttaaggtatagtccagccgttccagcgaaagcaaacgggtcaggcaatgggagcctgcggcttaatttgactcaacacggggaaac

SEQ ID NO: 114 - DNA, 417 bp, Graphium basitruncatum
  source 1..417: mol_type = genomic DNA
ggaagggcaccaccaggggtaaactgaagccgtatcgctctgccccgaaaagcgagcccgtaacgggttcggtggttgccggcattcgctaggtccccgtccaggcgggggcgacaccctcaacgtgcggggaaccctcgaagggcaccactcctaagtaacggtcgaaagacgcgctatggcggcgccgtaatggccgccggtacagggaaaacgtggtgcttaggaggcgattcgcagccaagctcctaccccgcctccggggcgcagggagaaggtccagagacttgacgggggtggggtgngcgcgcagcccgcgccnnngngcctaagataaagtccgtgcctgcgcgagagcgcgggaatgctctacagcagagccccgaacaggggatgcggcttaatttgactcaacac

SEQ ID NO: 115 - DNA, 409 bp, Graphium penicillioides
  source 1..409: mol_type = genomic DNA
ggaagggcaccaccaggggtaaactgcaaagccgtatcgctctgccccggaaagcgagcccgtaacgggtccagtggtcgccggcattcgctaagtctgcacgcgcagcggcgacaccctcaaattgcggggaaccctcgaagggcgtcactcctaaggacggtcgaaaggcgcgctatggngcagcgtaacggctgcnntacagggacaacgtgancctaggaggcgattcgaagccaagctcctaccngcgccccggcgccgtagggagaaggtccagagacttgacgggggtgggtaggtcctcgcgggcccgcctaagataaagtccgtgcctncgcgagagcgcgggaatgctccaagcagagccccggaaaggggctgcggcttaatttgactcaacacgggg

SEQ ID NO: 116 - DNA, 280 bp, Scotiellopsis oocystiformis
  source 1..280: mol_type = genomic DNA
ntcaacacgggaaaacttaccaggtccagacatagtgtaacgatagctgctcagctttagcgccagtggcttggaaacaagctgctagtcacactcccatctctaggcaagacagcctgcagtatggatgtggcaacaccctcaaattgctgggaagccctaaagctgctacataccaaagcttgttggaaacaactagctggccaggttaactacctcgggtatggtgacaattgtcccagacatagggaggattgacaagattgagagntntttctng

SEQ ID NO: 117 - DNA, 387 bp, Bensingtonia thailandica
  source 1..387: mol_type = genomic DNA
gcgttgattacgtccctgcccttttttcattttaaacgcttgatgtacaaggggttcaaagtgtcttggtaaaagatgctagtagtgaacacggtattactatttgtcaacttgccagaccgtcaaattgctggaaagcctaaagctttaactaccaagtcaagacagaaatgtactgatggctgggtcacgacccagggtaaggtgaaaacgttaaagatgtttaacaatgggtaatcagcagccaagccctaaggtgtcttgtacactagggtgagttnacagactagatgtcggtcggcttttcgattaatcgaaaaagcttaagatatagtcggcccaagtctgaaaggactttggataaagtgttgtacacaccgcccgtcg

SEQ ID NO: 118 - DNA, 437 bp, Gonatozygon aculeatum
  source 1..437: mol_type = genomic DNA
accttatcatttagaggaaggagaagtcgtaacaaggttttgcctcgagcaaacgcttgcggaagccttagcagcccgaaaaggttcccctcgcgaccaaatccgaaggaaggggattgaatgctagtgcccaagttgccaacttgggcgcgacatcgccaaattgcggagaactccactatttcgcctcgattaccaagcggtacgccgaaaggactccgtggccgggctagttgcctcgggtaaggtcataatatcgaggatgagggaagatccgcagccaagccctcgcgccaaacgctcgtgggtgctgttcacagactaaacggcggtgggttccggactctagaagcctggagcctaagatatagtcgttcgcctcgaaagagcaatctgcagagattcaccgttccgtaggtgaacctgcagaaggatca

SEQ ID NO: 119 - DNA, 439 bp, Mesotaenium caldariorum
  source 1..439: mol_type = genomic DNA
aacaaggtttggctcgatcgtggatcctttcgcggttgaaactaccgaagccttagctgcccgaaagggtgacctccgcgactcgcaaaacaaggagaggtctcaaaagctagtggctcctagctgctgctcgtgcagtgagtgcgacatcaccaaattgcgggaaatccctaaagctttgactaccaagtgcggcgccgaaaggctcgcatggccagggtaatgacctcgggtatggtaacaacgtcaaagatgcaacaatgggctatccgcagccaagtcctcgccgcttcgcagcggttggatgctgttcacagactagatggtggtgggttctcggatgcgtcccggggcttaagatatagtcgggcctgcttggaaacagcgggaaatcgcagatgcattcaccgttccgtaggtgaacctgcagaaggatcaa

SEQ ID NO: 120 - DNA, 414 bp, Staurastrum sp.
  source 1..414: mol_type = genomic DNA; note = M752
aacaaggttccgtttatgatccttctcgccccgttatgggttcgagtcgcacggaagccttagcactatgtgcattgtattttaatacgctgcacccgtgctagtgccaatgatgatgtcattggtgcgacgtcatcaaactgcgggaaagctcctatatggcatgggataccaagcacggcatcacacattgtgtgtgatgagtgtggctgagttaatgcactcgggtaaggtaacaacccccatgtcaagaggaccgacctgcagccaagcctttggttataccaagggtgctgttcacagactaaatgatgacgggttgctaatcacaaagcgtgattgcagctcaagatatagtcggactcactctagagtgagacaacacgtaataccttcccgcacgtgaattcaccg

SEQ ID NO: 121 - DNA, 417 bp, Cylindrocystis brebissonii
  source 1..417: mol_type = genomic DNA
tttagaggaaggagaagtcgtaacaaggtttcattgcgtccttctgcaagtttgctactgaagccttagctgcctgaaaaggtgaccttgacgacttgataaagaaacaaaggtcttaaatgctagtgttcaccctgagtggacgcgacatcaccaaattgcgggaaaaccctaaagctttgactaccaagtacacgtccgaaaggagtgcatggccagggtaatgacctcgggtatggtaataacgtcaaagatgaaacaatgggtaatccgcagccaagtccttgcttgtcatggatgccgttcacagactaaatggtggtgggtttgcgaagcaagcttaagatatattcgggcctctcggaaaccaaaagggggcagtttcnccgttccgtaggtgaacctgcagaaggatca

SEQ ID NO: 122 - DNA, 435 bp, Closterium spinosporum
  source 1..435: mol_type = genomic DNA; note = var. spinosporum
gaggaaggagaagtcgtaacaaggttgcccctgggcacgatccttccgaggaccccagacggaagcctgagctatctctgtccgcggcgtggagagagagagagctaggtgtggctcatgccataccgacaccgtccaactgcgcgagagttccagaccctggccggtaccaagcgctctcaagagcgtggccggagaagctgtcatccatcgcggctctcttcgggtatggtcataacccggcaccgggctcaaggataatccgcagccaacccgtctcaggcgggaccgttcacagactgaacgacggtgggtccatgcgtgcgtgggcttaagatacagtcggcatctccccatttaagggagattcgagcaagcacagtcggcctgcagcgatttcaccgttccgtaggtgaacctgcggaaggatcattg

SEQ ID NO: 123 - DNA, 445 bp, Closterium spinosporum
  source 1..445: mol_type = genomic DNA; note = malaysiense
gaggaaggagaagtcgtaacaaggttgcccctgggcacttccttccgacaagggacccagacggaagcctgagcaccgcccgctggcgcacagcgggctcggtgctaggcggtgcgcgcgtgcgcgcactgtcgacaccgtccaactgcgagagagttcccgaccgcggatggtaccaagcgcctcagagcgtggccggaggcggagcgccgctcccccctcygggtatggtcacaacccatccctggcagacggatgatccgcagccaacccgtcgcgtcgtgacgggccagttcacagactgaacgacggtgggtccgggtgtcgcaagccgcgaaacccgggcttaagatacagtcggcggctccgggagaaccggagtcccaccccctcccctcgcagaggacttcaccgttccgtaggtgaacctgcggaaggatcattg

SEQ ID NO: 124 - DNA, 359 bp, Phialophora gregata
  source 1..359: mol_type = genomic DNA; note = f. sp. adzukicola
ggtcatttagaggaagtaaaagtcgtaacaaggtttcaccacggagaccatgcatcccctgagggaggcagcgcgactgtaaataacgctgccgtatgcaagtcaggctaccgctctggcaacacgatcgaattgcggggacctcctaaagcttcactcactacctacccccgaaaggaggcagcacagttataacagtgaagatgttacaatggacgatccgcagcggagaccctacagcgcaagccacgggtagcgttcacagactaagtgattgtgggtagtacggtatactacttaagatatagtcgaacccttactgaaagtgaggggtaaaggtccgtaggtgaacctgcgga

SEQ ID NO: 125 - DNA, 443 bp, Dactylella gampsospora
  source 1..443: mol_type = genomic DNA
gtcgtaacaaggtttgaagtaagtttccttccataggtgacgctcacgagaagcctttgtggccccgcaaggggtacctgccacgactgtaaagaatggatgcaggtattaaatcgcaagtcagctagcagctggcaacacttttgaattgacggggacaccctaaagcccacctctaccaaccagctggagaaatccggttggggcctgtgttaacagcacagggtacggtaacaacgaggtcggataagccagccctctggcacaatgggtaatcctcagcccaagtccctaatcttttcgttcagaaaaagacatgggaaaggttcacagactaagtggaagtgggttggttcctgaacccaacttaagatatagtcgggctctgtgcgaaagcagagaggagtcactgttccgtaggtgaacctgcggaaggatcatta

SEQ ID NO: 126 - DNA, 349 bp, unidentified
  source 1..349: mol_type = genomic DNA; note = anamorphic fungus JCM 8069
gaggaagtaaaagtcgtaacaaggttttgctaagcaacagtccttctgtagcagagcttaacggagaccttgcgccaaaaggcgactataaataatcggcagcaagtcagtatcactggcgacacaatcgaattgcggggacgctttaaagctcgacggtaccaaccgctggaaatcccagcggggctagcatgctagaggtcagaactcgtcgagatgtacaataagcaatccgcagcggtccccttcgaggggccgtccacagactaagtggttgtgggtaggacgtctgttctatctaagatatagtcgggccttgcgagagatcgcaggggtttctgcgtccgta

SEQ ID NO: 127 - DNA, 410 bp, Encoelia fascicularis
  source 1..410: mol_type = genomic DNA
gcgtataggggcgaaagactaatcgaaccattggaagctactaggtggttaagaagagacttgaacaagtctcctagtcctattgtagggcaacactatcaaattgatcgggaactacctgttaaattctatgctaccgcagcctggtcgaaagacgggttgcgcaccaggttaacgaccttggggatggtaataatgcttagatagggtatatccgcagcgaaatcctaaggctcaggctatggatagcgttcacagactagacgatagtgggcttggcagctctatcttgccacagcttaagatatagtcgaacccttcagagatgcggggcgtgagtgatggatgcaagtctgttgctcagcgcatcagatgctagtagctggttcctgccgaagtttccctcagga

SEQ ID NO: 128 - DNA, 830 bp, Grovesinia pyramidalis
  source 1..830: mol_type = genomic DNA
gcgtataggggcgaaagactaatcgaatcatccacatggcatatatttatatagttgctaggtgattaagaagaggcttgaacaaggtctcctagtcctaccttagggcaacactatcaaattgttcgggaactacctgttagaattctaagctaccgcagctttgtcgaaagacggttgcgcaccaggttaatgacctcggggatggtaataatgcttagatagggtatatccgcagcgaaatcctaaggcttctataagctatggatagcgttcacagactagacgatagtggggcttttatgatattgaatcattagggcttaagatatagtcgaaaccttcagagatgaagggcatgagcaataggatatgaatacaatccgaatgctcatgcattacatgctagtagctggttcctgccgaagtttccctcaggaatagttgctaggtgattaagaagaggcttgaacaaggtctcctagtcctaccttagggcaacactatcaaattgttcgggaactacctgttagaattctaagctaccgcagctttgtcgaaagacggttgcgcaccaggttaatgacctcggggatggtaataatgcttagatagggtatatccgcagcgaaatcctaaggcttctataagctatggatagcgttcacagactagacgatagtggggcttttatgatattgaatcattagggcttaagatatagtcgaaaccttcagagatgaagggcatgagcaataggatatgaatacaatccgaatgctcatgcattacatgctagtagctggttcctgccgaagtttccctcagga

SEQ ID NO: 129 - DNA, 389 bp, Myriosclerotinia curreyana
  source 1..389: mol_type = genomic DNA
gcgtaaaggggcgaaagactaatcgaatcattaacactactaggtgattaagaagaaacttgaacaagtttcctagtcctatttcagggcaacactatcaaattgatcgggaactacctgctgaattctaagctaccgcagccttgtctaaagacgggtgcgcaccaggttaataacctcggggatggtaataatgcttagatagggtacatccgcagcgaaatcctaaggcctttgctatggatagcgttcacagactagacgatagtgggcttctcagtgagagcttaagatatagtcgaaaccttcagagatggagggcgtgagtatcttcgaatgctcatgcatcaaatgctagtagctggttcctgccgaagtttccctcagga

SEQ ID NO: 130 - DNA, 422 bp, Myriosclerotinia ciborium
  source 1..422: mol_type = genomic DNA
gcgtaaaggggcgaaagactaatcgaaccattaaggaatagaccaagctctaggtggttgagaaacccctttggtattagtcctggagacagggcgacattgtcaaattgttcggggaccacccgttaaattacatactaccgcaacaggcctgaaaggcctgcgagcactaggggtagcgcctctagggatggtaataacgtatgtatagggtatatccgcagcgaagttctaaggcctcacggctacgaatcgcgttcacagactagacggcaatgggctcttcacagggcttaagatatagtcgaacccctcagagatgaggatggaatcaatgctagtagctggttcctgccgaagtttccctcaggagcgtataggggcgaaagactaatcgaaccattaaggaatagaccaagctc

SEQ ID NO: 131 - DNA, 372 bp, Myriosclerotinia luzulae
  source 1..372: mol_type = genomic DNA
gcgtataggggcgaaagactaatcgaaccattaaggaatagaccaagctctaggtggttgagaaacccctttggtattagtcctggagacagggcgacattgtcaaattgttcggggaccacctgttaaattacatactaccgcaacagcgttgaaaggcctgtgagcactagaggtaacgcctttagggatggtaataacgtatgtatagggtatatccgcagcgaagttctaaggtcttacggctacgaatcgcgttcacagactagacggcaatgggctcttcacagggcttaagatatagtcgaacccctcagagatgaggatgggatcaatgctagtagctggttcctgccgaagtttccctcagga

SEQ ID NO: 132 - DNA, 370 bp, Myriosclerotinia caricis-ampullaceae
  source 1..370: mol_type = genomic DNA
gcgtataggggcgaaagactaatcgaaccattaaggaatagaccaagctctaggtggttgagaaacccccatggtattagtcctggagacagggcgacattgtcaaattgttcggggaccacctgtcaagttacacactaccgcaacagtgctgaaaagctgtgagcactagagatagcgcctctagggatggtaataacgtgtgtatagggtatatccgcagcgaagttctaaggcctcgcggctacgaatcgcgttcacagactagacggcaatgggtcctcgcagggcttaagatatagtcgaacccctcagagatgaggatggaatcaatgctagtagctggttcctgccgaagtttccctcagga

SEQ ID NO: 133 - DNA, 372 bp, Botrytis tulipae
  source 1..372: mol_type = genomic DNA
gcgtataggggcgaaagactaatcgaaccattaaggaatagaccaagctctaggtggttgagaaacccccttggtattagtcctggagacagggcgacattgtcaaattgttcggggaccacctgttaaattacatactaccgcagcagtgctgaaaggcctgtgagcactagaggtaacgcctctagggatggtaataacgtatgtatagggtatatccgcagcgaagttctaaggcctcacggctacgaatcgcgttcacagactagacggcaatgggctcctaacggggcttaagatatagtcgaacccctcagagatgaggatggaatcaaagctagtagctggttcctgccgaagtttccctcagga

SEQ ID NO: 134 - DNA, 373 bp, Botrytis porri
  source 1..373: mol_type = genomic DNA
gcgtataggggcgaaagactaatcgaaccattaaggaatagaccaagctctaggtggttgagaaacccctttggtattagtcctggagacagggcgacattgtcaaattgttcggggaccacctgttaaattacacgctaccgcaacagtgctgaaaggcctgtgagcactagaggtaacgcctctagggatggtaataacgcgtgtatagggtatatccgcagcgaagttctaaggccttcacggctacgaatcgcgttcacagactagacggcaatgggctcctcgcggggcttaagatatagtcgaacccctcagagatgaggatggaatcaaagctagtagctggttcctgccgaagtttccctcagga

SEQ ID NO: 135 - DNA, 371 bp, Sclerotinia sclerotiorum
  source 1..371: mol_type = genomic DNA
gcgtataggggcgaaagactaatcgaatcattaaggaatagaccaagctctaggtgattgagaaacccctttggtattagtcctggagacagggcgacattgtcaaattgttcggggaccacctgttaaattatatgctactgcagcagtgctgaaaggcctgtgagcactgagggtaacgccctcagggatggtaataacgcatatatagggtatatccgcagcgaagttctaaggcttcgagctatgaatcgcgttcacagactagacggcaatgggctcctcgcggggcttaagatatagtcgaacccctcagagatgaggatggaatcaatgctagtagctggttcctgccgaagtttccctcagga

SEQ ID NO: 136 - DNA, 370 bp, Sclerotinia tetraspora
  source 1..370: mol_type = genomic DNA
gcgtataggggcgaaagactaatcgaaccattaaggaatagaccaagctctaggtggttgagaaacccctttgtattagtcctggaaacagggcgacattgtcaaattgttcggggaccacccgctaaattacatgctaccgcagcagtgctgaaaggcctgtgagcactagaggtaacgcctctagggatggtaataacgcgtgtatagggtatatccgcagcgaagttctaaggccttctgctacgaatcgcgttcacagactagacggcaatgggctccttgcggggcttaagatatagtcgaacccctcagagatgaggatggaatcaatgctagtagctggttcctgccgaagtttccctcagga

SEQ ID NO: 137 - DNA, 291 bp, Nostoc sp.
  source 1..291: mol_type = genomic DNA; note = PCC73102
agataactgagccttgaaggagaaatccctcaagtggaagctctcaaactcagggaaacctaaatctggtgacagacatggcaatcctgagccaagcccgaaaattttagatttgcgattttagatttgggattagttttcaatctaaaatccaaaacccaaaattgagggaaggtgcagagacccgacgggagctaccctaacgtaaagtcgagggtaaagggagagtccaattctcaaaatctgatttggctattgccatcaggtagcagtgaaaactgcgggaggatg

SEQ ID NO: 138 - DNA, 363 bp, Gentiana ligustica
  source 1..363: mol_type = genomic DNA; note = Chloroplast
aattggattgagccttggtatggaaacctactaagtgataattttcaaattcagagaaaccctggaattaataaaaagggcaatcctgagccaaatcctatttttcgaaaaagcaaaaagaaaggcttagaaagaaataaaggataggtgcagagactcaacggaagctgttctaacaaatggagttggttgcgttggtaaagaaatctttctaccaaaaattccaaaaggatgaagaagaggggtgactgtatcaaatgattcgcccacagcctgtagatcaacggattaatcggacgagaataaagagagagtcccattctgcatgtcaatgccgacaacaatgaaattgatagtaagagg

SEQ ID NO: 139 - DNA, 407 bp, Draba tomentosa
  source 1..407: mol_type = genomic DNA; note = Chloroplast
atcggtagacgctacggacttaattggattgagccttggtatggaaacctactaagtgataactttcaaattcagagaaacccgggaattaacaatgggcaatcctgagccaaatcctggtttacgcgaacaaaccaaagtgtagaaagcgagaaagaagggataggtgcagagactcaatggaagctgttctaacaaatggagttcactaccttgtattgatcaaatgattcacttcatagtctgatagatccttggtggaactgattaatcggacgagaataaagatagagtcccattctacatgtcaatactgacaacaatgaaatttatagtaagatgaaaatccgttgacttttaaaatcgtgagggttcaagtccctctatccccccaaacagtctgtttg

SEQ ID NO: 140 - DNA, 407 bp, Draba dubia
  source 1..407: mol_type = genomic DNA; note = Chloroplast
atcggtagacgctacggacttaattggattgagccttggtatggaaacctactaagtgataactttcaaattcagagaaacccgggaattaacaatgggcaatcctgagccaaatcctggtttacgcgaacaaaccaaagtgtagaaagcgagaaagaagggataggtgcagagactcaatggaagctgttctaacaaatggagttcactaccttgtattgatcaaatgattcacttcatagtctgatagatccttggtggaactgattaatcggacgagaataaagatagagtcccattctacatgtcaatactgacaacaatgaaatttatagtaagatgaaaatccgttgacttttaaaatcgtgagggttcaagtccctctatccccatccccaactatactc

SEQ ID NO: 141 - DNA, 290 bp, unidentified
  source 1..290: mol_type = genomic DNA; note = T4 td
gatgttttcttgggttaattgaggcctgagtataaggtgacttatacttgtaatctatctaaacggggaacctctctagtagacaatcccgtgctaaattgtaggactggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatatt

SEQ ID NO: 142 - DNA, 437 bp, Tetrahymena thermophila
  source 1..437: mol_type = genomic DNA
taactatgactctctaaattgcaaaatttacctttggagggaaaagttatcaggcctgcacctgatagctagtctttaaaccaatagattgcatcggtttaataggcaagaccgtcaaattgcgggaaaagggtcaacagccgttcagtaccaagtctcaggggaaactttgagatggccttgcaaaggatatggtaataagctgacggacagggtcctaaccacgcagccaagtcctaagtcaacatttcggtgttgatatggatgcagttcacagactaaatgtcggtcggggaagaataggtattcttctcataagatatagtcggacctctccttaatgggagctggcggatgaagtggtgcaacactagagccgccgggaactaataataaacataatattagttttggactaatcgtaaggtagccaaatg

SEQ ID NO: 143 - DNA, 846 bp, Paraburkholderia xenovorans
  source 1..846: mol_type = genomic DNA
tacgcctatggtgcgacacgaagcatgaggctcgttgttgaagagcgcagtgcatgtccgcttcatcattgtccagtcgcgaacatgttcgtacgatagaggacccgctgtgcgaccagaggtggcctaggaaagcatctttcgttggcgacgacgctggatgaagcctttccgacaacgtagtccaactgtatgcgccgccgcgaggtgacgtgtagaagctctgagcagcgacggggccgagacgcaaggctggccggtatgggtaacacatgtgaatcgctgataaacgtcgtcagaagaagaatgccaaagctgctgtcaggcatttaccaaacaggtgcacgggcggatgctccgttcataggtcggagcacgtcgtcgagatgcccgccggcgaaaaggcggaccctaacccggtcgtgtgatgaagcgggaacgtggtaagcccatacagttgcccacgaggcagacgagtcgcaaggcgagttgttgactgtgcgggtaagagaagacggaaaaagcgaatgctgggctgaaacggtccagataagggtttcaacattaccctacgcgaaagcgtgcagacttccgtccggtgtccagcgcaaagctgtcgaaagacttcgtagatggaagcatgaaagagcatcgttcgcggtgtctgcgcccttcgctgcggcggaagaacctcggcaagacaatgcgcaggcgtcagatggtggacgccggtcacaccggcgtgtgactttggaatgcttgagccgtatgcagggaaacttgcacgtacggttcttaggggaggccgcagcggcgacgctgtggccttacccgattttaagcccg

SEQ ID NO: 144 - DNA, 642 bp, Methanosarcina acetivorans
  source 1..642: mol_type = genomic DNA
gagggtcatggtgtgccacgaggctgcagaaaagatgagggtagacccctcggactcgctttacaggaagttattccaaaccacccataagctgctttggtcaaaagccacggtagtgagcattatgggaaaaggcgaggctatgacttcgtcaggtgtgggttaaggagccgaatgaaaatgaaccactgaagacgtatcgaaaacgcgatgatgtcgtcaaaaccaggtggcgtgactaacctgggataagtctggaagatactctgtttactgaccagatggtgaccggtatataggtggcgggaacttaactcgtgcttttctgtggaacgtgggaacctgctcatcgatgtaaagagaaacctccaagcggtggacccgcaaggacaaaagtatcgatgcgatgaacaggggcggaacaattcgtagtagtgatgaagtgcctgtaatgggcatggagcgaagggattgtattatccagttttgaactgcggtcaaccttgtcttcaagggaggaaccaatggacaaaacaaggctttatggctggataatgggagccgtatgaatcgagagattcaagtacggatctgagagggcctgggggtgaaattcccctgggctacttaccaatgcccaga

SEQ ID NO: 145 - DNA, 859 bp, Thermosynechococcus elongatus
  source 1..859: mol_type = genomic DNA
agacaaggcagtgcgacgcgaaagctagccagatgattgtcccactagcccaacaagctagaacgggaccggttgttcccccaaccgtagcctaaggaggcatgcgtgactggtaacggtcaggtatgaagccctcccgacaatgaagcccgaaccggaaggttggagccgaatccgtgaggaggaagcaacttcaccagcgtcaggtgatagggagctaggcttgagggtatggtgaacgcaagtgaagtgacgctagaagcctcgttactccaagcaggccaaagatgctgataggcctgagccaaaatggcaaagcggattggattcactgcttgcttttcagtgaacggggacagcaactccgccggggtatagtcaccacctaacccctcgtgtcatctggttggaacgcggtaagcccgtatcttcgccttgaacattcaaggcaggcaaaccgtaaggaatgctgatgggggtgcgggtagaggaggtgggaaaaagcgaatgctagcctgtaatgggctagatagggattgagaatgctggcaggacattcaacatcatcccactcgaaagagggcagacttcccgccggtccccccttacgagaaagtctatagaaccttcctaatggtgacaatgcaaatggcggtgatcttgagtcactggtgcggtcaccaacctgctcaaaaggtagagaggctgtagatgagtatcgtaaaggttgcatgcacaaccggttcctcttaaatgaggggtcctggggggctcgagccggatgcggggaaacttgcacgtccggttcctagggggctagggggcagcgatgcccccctgctacccgacaatgacggtg

SEQ ID NO: 146 - DNA, 2487 bp, Bryopsis maxima
  source 1..2487: mol_type = genomic DNA
aatgcatgacgtgcgacacgaaagtcgctgtatttagtagatatttacaaatcttatcctagcattcggatattcccatactttgggtgcaatcctataataataaaagattgtgctaagccttagtaaaagccaaaacttatgatgttctcgtaatataaggcaagggaaaatagaatagagttggtttaacggcctcgtaaagatgagaacagcccaaaagggttaactacttcgctaacagactgaatggatcgtcgtggtcattcaggaatgcttctaagaagtcggggtgattgtctccaaaactattctatgtatctaaatatagtggaacgtgggaaatccaaaaatctccctatgggtaggtgtttagcaataaacactaaaagattttggattgcggattgtcctgttatgtaagaattccatgcaaaggaataatgaacccatagcaaaaaggaaaatatgggatgcgaattactcagtaaaaagtaagttctaatacagaaaacagcccatatataactggaattcaggaaagttatttccaacaaatgaggacaaggtcttatttcataaaatttatattaaaataacaaagaacaaaggagattgacttatgatagtactttgggagactcaccagagtggataccatccaattggaacaccacaatggcacaagttcggaatttaaaatatagaatctttatggcgaaactagaaggtaatcggagaacattacgaagattacaaaaattgatgttagatagtcgccacaagatactcctgtccgtgagaagggtaacggtgattaatgatggaaaaatagattcctgggatagataaagttctaataaaagataaaaatgacagaatggtattggtagaaaaactctataaaatgaatattttacaatacgagcctaagcccgtgaagagaatacaaataccaaaatcaaacgggaaaactagacctctgggaatccctaccatactagacagaatgtatacaagccatagtgctaaatgcattgcaaccagaatgggaagcctgttttgaagcaaatagttacgctttcgcccaggaagaagtgcacacgacgcgattggtcgggtgttcaacgtactaagtgttaagtcaaatggaaacaataataaaccatggatattagatgcacatatcgaagggtttttcgataacatatgtcatcaagacatattagaaaaactcgacaacttcccagccaaaaagcttataaaccgttggttaaaagcaggctacatggaagaagatatcttttcaaaaactgagaaaggtacaccgaaaggatccattatcagcccactactcgcaaacatcgcattgtatgagttagaggaatttcttaaagcatcaccagactcaacaggtcgagtgagggggaatagagtatacgttcgatatgcagacgacttcatagtcatatgcgcatcaaaaacggaagctcaacaaaccaagaaacaaatctcaggttggttaaaaaccaaaggtctcagatttgcgccaaataaaataaaaatcagtgatattgataaaggctttgaatttttaggtgtagatattcgccatgtaagtacgaatgcctcgcataaaaccaaaggtaaaatgcttctgatacgcccctctacaaaggctatcgacaaaataacacaaaaactgaaaaaagaatggtttctcccgagaagtaaactcatagaaacggtactcacacgtctaaacccaatgatcacgggctgggcaaattaccatcaaaaattttcatccacggatatctttaataaattagacaactggacattccagaaaagttggaattatgcatccagaatgcacgccaacaaatcgaaacattggatttatgataagtattatgggaacttcaatttaaaccgaaaggaccgttggatattcggagataaggaaacaggaagataccttaaaaagtttgcttggttcaatataaagagacacgtggtggtaaaaggctacaattccccttgtgacccaaggttacaagaatactgggaagaacgccggaaaactctctataaagtcagtagaacctaatcggaacaaataatcgcgaaaaatcaagattatagatgtccagtttgtgaacacccgatgagaatctacagaaacatcacctaatttcccaacgaaatataaaaattgaaaattattggaacctagtatatatcatagacaatgccatcaattctgacattccaacccgaaaattgaacgagaattacatggaaaattttattcgaggaaaatcgtgttcgtaagcgtctaacaaagaatccgcgaacacacatagaaagattaaaactcgaacttttagaacataaggcttgagccgtatgcggggagactcgcacgtacggttcttaggggggaattggtggaaacgcctgacctacccgactatttaactg

SEQ ID NO: 147 - DNA, 1858 bp, Methanosarcinales archaeon
  source 1..1858: mol_type = genomic DNA
cgttactcacgtgcgccacgaagctgcacaagagatgagggttcggcccctcgaagcctgcttgcaggagccggcttcaaaccgccttaagctgctgtggtcaaaagccatggtggtgagcgttaaggaaaaggtcccacaagatgggatcaggtgtagatcatggagacgaacgtaagtgaaccgctgaggaagtgtcgaaagcgtagggacgacgtcaaaaccaggggctggtcgttgacctgggataagtctgggagagcgcctgcttactgcccagatggcgtccggcatgaaggcggcgtgaacatggtcctggctcttgtgtggaacgtgggaacctatcccctaatgttaagggagaaacacaagtgggaggaccccacaagtgtgagagtaccgatgcaggggaataggggcggaccagcccgtattagtgatgaagtttctgtaatggagatagagcgaaggggttgggtcatcctgcttgatttaaagatcaaccaggagatgggaggaatctttgatgaaagcaaagccatttaagatttccaagaaagttgtattggaagcgtggaaagaggtgaaagcaaaccgtggagcagcaggtgtagataagaagtcgattgcggattttgagaaggatcttaataataacctttacaagatcaggaatcggatgtcatcggggagctactttcctccaccggtgaggacggtagggataccgaagaagagtgggggagagaggttgttaggcatacccactgtggctgacagagtggcccagacagtggcaaagatgtaccttgagcctttggtagagccatactttcacaaggactcatacgggtacagacctggtaaatcagcgattcaagcagttggagtgacgcgtaagcgttgctggaggtatgattggatgcttgaattcgacatcaaggggttatttgacaatattaatcataacctcttgataagggcggtaaggaaacacacaaactgtaaatggatgcttttgtacatagacagatggttgaaggctccatttcagagacaagatggtacgttagtgcagagagaaaaaggaactccccaaggtggcgtgataagccccttgcttgccaatctcgttctgcactatgtgtttgataaatggatggagcggaactatccgcaagttccattctgccgctatgctgatgatggtgttgtgcactgccggtctgaagccgaggcattaaagctaaggaaaactctgggggctaggtttgggaagtacaatcttgagcttcatccggagaagacgaagatcgtctattgtaaagatgatgatcgtcgagatgactatcccaacacgagttttgattttcttggctttacattccggccaaggaggtcaaagaatcggtggggtaagcttttcatcaactttactcctgccgtgagtaataatgcggcagaggcgatgaggcaaaaggctcgaagatggaagatgcacctgcgaagtgacaagtccttggacgatctgtcaaggatgttcagtcctattatcagaggctggatcaattactatgggcgcttttataaatccgcgctgtacccaactttgcaacatctgaacagaacgttagtcagatgggcaatgaggaaatttaagaggtttagacaccatcgtaggcgtgcggaatactggttaggggaaattgctcaaaggcagcccgggctgtttccgcattggcagatgggtgtgagacctacggctggatgatgggagcccggtgagctgagaggttcacgccgggttctgagaggacgtaggggtgagattcccctgcgttactcacctgcgccacga

SEQ ID NO: 148 - DNA, 1529 bp, Marchantia polymorpha
  source 1..1529: mol_type = genomic DNA
aagagttatcatgacaaaatgcaattacgagcaactactagaccccgagatatttaggctagcttacgagctaaagaaatcgaaatcaggcaatatgaaacctggtgcggataaagaaactctcgacggtttctcccaagcctatgttgagaaggtcgtccgtcaactaaaagacgaatcatttcaattccgtccgtcacgaagagaattcattcctaaagcagacggcaagcttcgttccctgggcataccatcacctagagacaagatagtacaagaggtcatgaggaggatccttgaacctgtatttgaaccgcgattcctggattcgtctcacggatttagacctcatcgatcaccgcacacggctctacgacaaatccgtcgatggacaggcacctcctggatgatcgaaggagacatcaaaggatacttcgacaacatcgatcatcacctactcgcgggattcatagcagagttggtaaaggaccaacggcttctcgcgctttattggaaattggtacgcgctggctatgtaaatcaaggcaaagcagagccacacttgctaacaggagtacctcaaggaaggatactatcgcctctgctttccaacatctacctacaccagttcgatctattcatggaggaaatcaaagtcaaatatacaacgaccggtgcgctttccaaaaacaacccgatttacttgaaggcgcggaataaatactacaaacttgtgaaatcattaaaggcttcttccgccgaaatcatccgagcgagacgcgatatgttgaaaatgacttacgggattcaaacaggttctagggtgcgttatgttagatacgcggacgattgggtgatcggggtcacgggtccaaaagccctggccgtacaaatcaaagaagaggtctctaccttcctccaagaaaaactaaaactttcgcttcaggccgaaaaaacacgtattaccaatctatcaagaagcgaagctttattcctaggaaccttaataagcataacaactcgtaaatacgtgcaaagccagaaggtgggcggggggcacaggcgagcctccctcggtagaatacgcctgtgcatccccatcgatatccttatcgggaagctctcacaaatgggggcgtgcgacgaaaagggaacgcccaaagcggtgaccaaatggatctttctaaacgtgggagaaataatcaacaaatatatggctgtgttccggggatactacaactactactcattcgcagacgatatccatcaccttctccaaataatatacatactaagatactcggctatcaacacggtcgcccgtaaactggggcttaacacagccaaagtaataaaacgcttcggcgtggacctaatcttccgggaccacacgaatgagatcaagcataagctcaatttcccgcgatccctacctaataagcgcatgaacttcgccttgagtccgccttcggaccctagagttctatttgatacaagctgcgctcgcattcagtgttgaacgacctgtg

SEQ ID NO: 149 - DNA, 1910 bp, Vibrio harveyi
  source 1..1910: mol_type = genomic DNA
ttttcttttggtgcgccgagcatggcgttgatctaggaggtgaaagtcctctacgggctcagtcgagcgagaaccgttagcctatgcaagggtgttcactgtgaggtgaaatctgaaggaagcaaatggcaaaacttggttttgacgaacagaaatctgatagtaggtcactacaacttgggcaacctagccatagatagaatagcccaatgcctcgacgggaagtgtgtagtgataaatcaggcacggccaagggaaagagcaacgtcttacctcgggagatcttattgtttgtctcgatagagactaatgcagcagtgatgttgtgtgacgggcaataagaagtcagcagagggcatagtaccttgagacagtacaacttaagggaaggcctgaactgaatatatcaagaagtaagtcactagacactcaatcatgtggagtcatagcaagatggacatcatctctacgtcccaatgggcgataccgcaagtaaagctcacggtgacgaagaatggcaagcatggtcggcgtagacaggaggacgagtcttggtgacctcaacttcgttgatggaacaaatctgctcctcaaccaacctgaatcaagctttacgaagagtgaagaagaacaagggatgtgcaggtgtcgacaaactcgacatagatgcaactatctttaagcttcgacaagcttcaaacgggcaagcgcttcgccagagtcttctggatggaagctaccgacctcaacccgttcttggtgtgggaatccccaaaccgagtggtggcgtcagacaactaggtatcccaacagtgctcgatagaattgtccaacaggcaatcacctcagtactgtcagatatctatgaagctaagttctccaatagcagttacggatttagacccagccgcagtgcacatcatgcgctagcggcagccagtcgctacatccgagaggggcgaggttatgtagtagatatcgacctagcgaaatacttcgataccgttaatcacgatagactgatgcacagactgtcagaggatatcgcggataaacgagtattgaagctcatcaggtcatatctacaggcaggcataatgcgaaacggtttagtcgaacagagacaacgagggacaccacagggtggtccattatctccgttgctatccaatatcgtattagatgaactggataaagagctagagcgtagagggcataagttctgtcgatatgcagacgactgtcaaatctatgttggtagtgaggaagccgcttatcgagtgaaagagtcgataacggagtacttggagcaaaaactgaaactcacggtaaaccgagagaaaagtgcagcaacaagagtgacagagcgaacttacctaagccaccgcttcgggatagatgggacaatccatatatcgaagccagcgcaaactcaaatgaagacgcgagtgcggcagataacgaagcgaaaccgaggtcgagagttaaaggtgataatcgccgagttaactcaatacctaagaggttggcaagactacttcaaactcgccatacggaagagcagcatgaagcgcttggacgaatggatacggcgacggctaagatgctatcgacttaagcaacgcaaacacagatacggcatagcgacatggttacagcgacaaggtgtaacagagcgtaatgcgtggaagttagcgatgtcagataaaggttggtggcggctagcattaacgccccagacaaatcacgctatgccaacaaaatggttcgaggagttgggtctgtactcgttgcgagatgggtatgagtcactaaaagtatattcggaaccgccgtatgcgacccacgcttgtacggtggtgtgagaggacggaggccgtgaggcctcctcctactcgattattgccttt

SEQ ID NO: 150 - DNA, 2742 bp, Crocosphaera watsonii
  source 1..2742: mol_type = genomic DNA
ttaggggatagtgcgattcgttgagctagaaagctgacatccaagtatattacaaggatacagcgtgtagctcgttcaagagccttttaaaaggtaatccaaaggttaaatgaccaatcaccctccgaagaatcaccctccgaagagaaaacaaagcctagagcgcattctttaaaaggggtaaatacgaaggtcgaatgggtaaaacaacacccaacaaagcaataactaataaagtttacataacttaattagttattaccgaactagctaagtagggataaccgaacagccaatgaacctggaaacttgaataactgttggtttgttggcagaaaggagccacactcggaatcaagggataactcatcccaagagtgacgacgtaaccctccgttttggaattagtaaacccaggatggaagcagggttaaaaggagaacccgtcatgaaacagcatttcggagtcgttctttaagtcaatcggtcatggttaaacattacgttgaatcatcgtcctgaaccctcatgaatactataagtagccaaatctgtttgataggctgcgttcaaaagaacaaggtgaaaagtggagagtttgtcccctcatccacacagattcctaaaaataacctgggggagagatgaaacctaaaaccgatatacccaaatcaacggaacgaattaagtccatgatttctcttaccatcaggtcgatgatgtaagtagtgacaaacttaagcgcaagggtcatggatgtgagaagcaataagaagcaaatgcctacttgtaatgagtacggataagctgacgtattgctggttaattagaagaaatagtctctattagaggtagttaaatgtcgaatgcgagtataaccaagactacgccagaatggaacacaatcaactgggcaaaagtccagaggaaagtgtttaagcttcaaaaaagaatatttcaagctgttaaatcaggtaataaagttaaagctaaaaagcttcagaagttactgttaaagtcacactacgcaaagctcctagccatacgcaaagtgacccaagacaaccaaggcaagaaaactgctggagttgatgggataaaggcattacgccccaaccaaagactaaagctaattaaagaacttaccttaaaaggttacaaagcaaaggcacttcgacgggtttggattcctaagcctggaagagaagaaaaacgtggactgggaataccaaccatgaaagacagagcgatgcaagccttggttaaatcagccttagaaccatattgggaagcccaattcgagggaacatcttatggtttccgtccaggcagaagcgcacatgacgctatcggtagaatatactctggaatcaatcaatgttccggtggtaaatatgtcttagatgccgatatagcaaaatgcttcgataagattaaccatgagtacctactgtccaaagtagattgtccatatctcatcaaaagaatcattaaacaatggttacaaagcggggtaatggacaatggtgtatttgaggaaaccgattcaggaacacctcaaggtggtgtaataagtcccttgttggctaacattgcactacatggaatgataaatgacacaatgaacaagtttcccaaatccttttacagaaatgggaaacaagtgagggataatcaacccaaaatcatccgatacgcagatgacttcgtaattatacataacgaatacgaagtcatcctacaatgtaaagaaataattgcacaatggttaaaaaaggctggactggaactaaaaccagaaaagacctccatcagacataccttaaaaagtattgaacacgatggacaaaccgttgaccctggtttcgattttcttggattcaacatcaggtcatattcaaagggaaagcaccgttcaggtaaaaccccacgtggcaagataataggaagcaagacactaatcaaaccaagcaagaaaaaaatactagctcaccacgaggctttaaaagaggtcattaaagacaataaaaaagccccacaagcagcattaatcgcaagattaaatcccataatactaggatggtgtaactactatcgaacggtcgcaagtaaggaaacatttgcatccgaaaacaacgtactatggaatatgctaagagcttggacagttaacaggaaaaagaaaaaaacaccattgtatgaagcccttagaaaatatttctcatacggaaagtacggaaaatggacgtttcaaacagaagaagcagtcctctactatcatgccgaaacggaaataaagagacatcaattggttaaacctgacgcatcaccttatgacggaaactggacttactggagtaaaagacggggaacatataccgggacaccagctagagtatcaagactcttaaagaaacaaaaaggtatatgccctcaatgtaagcaacacttcacacctgaagacttgatagaagttgaccacattatccctaaatcgaaaggtggtttggatacatacaacaatcttcaagctctacatcgtcactgtcatgaggccaaaagcaaaaatgactacctctatgattggcacttataatggctatgaatggaaagacgatgtattaacagtaccctagacaagggactaattgccgagagccgtgtgaggtgaaagtctcacgcacggttcggactgggagtcgtggtaggtgactaccacttcgacccctaatcaagcttatt

SEQ ID NO: 151 - DNA, 2038 bp, unidentified
  source 1..2038: mol_type = genomic DNA; note = Candidatus Kuenenia stuttgartiensis
cgttaattcggtacgccaagaagcgtatatatatcagttggtgcaaatccaacccgccaattgaccgttcgggcggtagtgaaagaggcagaaaacctcacgggagaacgtaaacagcgatgaatacgttcgagtcccctgtagcgagcagtcagaccgtagcgcaagcgaacacgcaggcttcgttacgctaaaaatacgatagatgagcctcccggattgggcgaaatctgtaatcgcataggtagaaagagacaaacggtagaactatgcggtgtcgtcggagttgagagtgacggtatgactgtaaaaggtatatatatgaacttgggaggtctcattgttcaggttgatgaccgtaacgcatactataagggaagccgaagggtatagcgggataatgagaagtcggagatgcccatagtagtgaagatggtgaagacaacataacttcaccgtagcgaaggggcattacttcagttaacgtttacaaagaaaggaggaaattgtattgggagacctacaagcaataccgaaatcgcaaatgagcgatgatgaaagagtccgagactttcagcgtaaactatatcgaaaagccaaacaggaagaaggatttcgcttttatgtattgtatgacaaggtgagaatgttgcactttttgagagaggcgtacaagcgctgcaaagccaatggaggaagcgccggggcagacgggattacatttgaagatgtggagtcatacggggtggagaagtttcttggagagataatagaggaacttgaaaacaaaacctacgaaccacagccggtactgagagtgtacatacctaaaaccaacggtaagacacgaccattgggaatccctgtgataaaagacagggtagtgcagatgagtgtaaagcttgtaatagagccgatattcgaagcagactttgaagatagctcctacggattccgaccgggacgttcagcgggcgatgctgtgaggaaaatcaaagaaaaactgcgagaagggaaaacagaggtatttgacgcagacctgagttcttattttgacacaataccgcacaaggaattgttactgttaatagggatgaggataagcgacaagaacgtactgcacttgataaaaatgtggttaaaagcaccggtgatagaggaaggcaaaccaggaggcgggaggaaaaataagataggaacaccgcagggaagcgtaatatcaccattattagcaaacatatatctgcacatgctcgacaaagcagtcaatagagagaatggggttttctataagtatgggataacaatcatacgttatgcggatgattgggtattgatggcaaagcgaatacctcgtgaagcattagactatctcaacaggttgttaaagaagttaaagctgagtcttaacgaggataagagtaaaatagtaaaagctgaagaagagagctttgactttctcggacataccatatccttctcagaagatttgtttggtaggaaacacaagaaatactggaacatagaaccaagcaggaaatcacagaagaaggttagggaaaagataggaaactacctaaagagcaatgggcataaagcggcggaaaaagtagctaatgaactgaatgcaataacaagaggatggataaattacttcacaataaagggagtaacgtatccaaacaaggcgaaaagagacctacgatattatcttttcaggagattaacgagatattataagaggaaaagtcagcgcaggagcaagctctataatcgaggagcgtttaaggaattagtcaatagatattgactaatagacccgacaaaatatgttcctgccagacaacctgtgaaagcttaagaagaagactgtcggaaagccgtgtgcgggaaaattgcatgcacggtttgacgaggggggcagatgataagtatatggcggagatatggtggcactggcgggaaaccagccagcaaacagagaaaacaaacttctgcctaagttctagtcgtctgtttctactctaccgttcattag

SEQ ID NO: 152 - DNA, 2003 bp, unidentified
  source 1..2003: mol_type = genomic DNA; note = Candidatus Accumulibacter phosphatis
agcgccgggtgtgcgcccagcatgggcgcgatgtgatggggtggaagtcccctgtgggagtaccgacgtgacttcatgatgaagccatgataaccactagccgaaggcaagggcaaggtcgtgaggacttgtctggaggaagccggagcgcaaaagtgcgaggcgacggacagaaactgcatagaaggcctggtctgcgggcgagttggcaccccatgacgaagcccaccggatacggcggacaaggtaaatgcagcgcttgtgcacggaaacatcgcgttcttacctggggagacctgatcggcaagcgattgccgacgtttcgcaaaagcagtggggatcgctgcttaacagaatgatccgggcgttgcgggaggctgccggccgcagacgagcgaacaggcagcgagcgaagcgaaggccatagcgcgacggcgagcaatcgccagcgtgactgatcaggagtcagcagaggccatagtagcgacaccgaaaggtaacgcgaagggccgaacatgaacagacaaggaggagccgtgagtcactcgaacccgacaatgaacccgaccgggggagccggggatcggcgtgatcttgcccagccagccttgcacgacgccccgaaggaaggttgtatggggaaggacttgatggaagccgtactctcaccggccaacctgaaacaggcgtggcgtcgagtgaaatccaatcgcggcgcaccgggaatcgacggactgcgcattgaggacttcccggcgtatgcttgtgagcactggccggccatccgccagaccctgagcgagggtcgctatcagccacaagcggtgcgacgggtgattatcccgaagcccaatggtggcgaacgcgcgctgggcatcccgaccgttgttgatcgggtcgtccagcaagccatcgctcaaatcatgacaccgatcttcgatccggaattctccgaatcgagctacgggtttcggccccgacgctccgcccacggcgcactcaagcaggtcagagccgacctcaaggccggctatcgcatcgccgttgatctcgacttggcgaaatttttcgacaacgtcgatcacgacatcctgatggcccgagtggccagaaaggtgagcgacaagcggttgctggccctcatcggccgctacctgcgggcgggtgtcatgattggttccacgcttcaacccagcgagttgggaacgccgcaaggcggacccctctcgccgctgctggccaacatcctgcttgacgatctggatcggactctggaagggcgaggccaccgctttgcgcggtacgccgacgacctcatggttctggtcaagagcgaacgggccggccagcgcgtcaaggccagtctcaccgcctacctcggccggcaactcaaactgccggtcaacgagaagaagagccaggtggcaaagatcgagcagtgcgtttttctgggcttcaccttcaggaagaacaaactgcgctggtcggatgccgccttcgccgacttcaagcaccgcctccgggaactgacggggcgaagctggggagtctcgatgccacatcggttcgagaaactcgggcagtacctgcgcggctggatgggctactttggcatctcggagtattaccgaccgattcccgaactggatgaatggctacgccgacgtgtgcgcatgtgctactggaaacagtggcgtttgtgtcgcacgaagatcagccaccttctggcgctgggggtcgaccggcggacggcgatcttgacgggcgtcagcagcaagagctactggcatttgtctcggtcaaaggccacgcaagtcgggatgacgaacgattggttgagagcacaaggtctggtgagtatccggaatctttggatgaaggctcacggttacgcttaacaacgtcgtgtgcgccctgttgatgaaccgcccggtgcggacccgcatgccgggtggtgtggggagggccggttagacgccggcccttacccgattagggcctga

SEQ ID NO: 153 - DNA, 2557 bp, Bordetella petrii
  source 1..2557: mol_type = genomic DNA
ctgcggcaaggtgcgccgtatcggcgcttagaagaagcgtagcgcaagctaccagggctactggaatgccctgtactcaccggacttatccctgcctgcaagggcttgaattccgaaaggaaggggggagtgcagcatgttcggaaaaggccttttacccaacagctattagcgggggcgggccagagtaagacgtgtatggtcaaagctgaattgcctgaagtctagttccaagcggtctaccagtcccgacgctggcgaaagtagctgaaacgccagactcgcgagttgtgggaaatgttttgcctctcccacaaaccagtatcgcgagcgcaaccccgatcaggggattacggaacgaacggacgacctaaccacgtcgcatctttgtctaagcgtaaatgcgggatcgcctaaacaggtcgtaagacctgcatggcgacggagtcttcgtattacccgatgtggcctctgtaatggaggttgctggccgcagcagtaagcggcaccgacatggctataagcgagcagcaatgcgcgtgaaaggcccactgtccggggggaaggaagacagcttatctgtaaccaactcatcgaaaaggaggttgtgctgatgctaaccgacactactaatcgaaggttggatgcgctgggaatcttgtctcaacagggcaagcgcatcaacggccttagtcgtctcatggaaaacccaatcctgtggaagcaggcgtatgtcaacatctatgcgaattccggtgcaacaacggccggcgtagatggctcgtcactggatggtatgtcctacgagcggctggccggcctgatggccgccgtcaaaagcggtaattatcgcttcaagcccgtgaggcgagtcctcattccaaagagcaatgggaaaacccgtccgctcggcatccccactggggatgacaagctggtacaggaggttgtacggatgcttctcgtgaagatctacgagccggtcttttccgacgactcgcacggttttcgcaacgggcgttcgtgccacaccgctctcatgcaagtccggcagaaatggacgggcatgaagtggatcgtgaacatggatatcaaaggttacttcgacaacatcgaccatgaagttctggtggatgtgcttgccaaacgcattgacgataagcgctttcttgggctgattcattcgatgctgaaggcgggctacatggaggactggaagttccatgacaccttcagcgggacaccgcaaggcggagttgtctccccggtcttggccaacatctacttgcacgaactcgacgagtacgttgcagggttgaaggccgaattcaaccgaggaaaccgccgggcgtcgaaccgcgagtacaagcggatcagtggtgcaattgaacggttgatgaaacgcattgatgcctacaaagccgatggcgactcgccaaaggtcgaggaagcgaagcgtgaattggccgaactttatctacgccgcaaggcactgagcagctccgacccgatggacgccaattaccggcgtctggtgtatgtccggtatgccgatgattttctgatcggcatcatcggcagtcgggacgaggcggtaaccgtcatgcagcgagtcgcaggtttcatcagcgacaaactgcacttggagatcgctgaggaaaagtccggtgtggttcacgcatcggacggagttcgctttctggggtacgacgttcgcacgtactccggtgatcgcaacgtgcgcaccgtccgttcaggacggagcatcacagcacgcagcgtatccgagcggatgcaattgcacgtaccggctgaaaagctgcgtagtttctgccagcgaaagggctacggcgtctatgacgctcacacccccacgcacaggagcgaactgatctcactgagcgaagcggaaatcgttcaaacctacaacgctgaaatgcgaggccttgccaactactacggtttggcgaacagcgtcagtcgtgtcctgaacaagctccatcacctctggttgacgagtttgtggaagacattggcggcaaaacgtcgttcgaccgtcacgaaggtagcgcggagcatgaagcgccagggtggatacgtactggctgttcggggtaactcggactcccacacttttcccatctacagcctgaaagacataaggaaagaacccatcacgttccagagtattgacctaccccccaagacttgggttttcacgcatagccgctcggagttgatccagcggctcgaagcgaggaagtgcgagtactgcgggaccaccgaaggcagtttccaagtgcatcacattcggaagctgaaggacgtggacaaagggaagtacctctggccacaggtgatgtcatggcgccgtcgcaagactttggtgctgtgcgttccttgccatcagaagctacacgcaggcgttctttgaaaggactgttcctagtcaagtaagtggagagccggatacgctgaaaggtgtaagtccggttcggaggggggctgacgggtgatcctttacgggacgcccgcagcctaccctacacgaccgtcg

SEQ ID NO: 154 - DNA, 1957 bp, Cupriavidus taiwanensis
  source 1..1957: mol_type = genomic DNA
agtacgattagtgcgccaggagactggcagagagtagtcgatgaaagttcgatacaggaaaggaatagcgaaccaccctggccccgagtcatgcggcggcacccgcgagggtgcagccgaagcgttgacaggggagagtgcaggccagccattgagccgcgaaatcattctgtctggagcgccgacgctgttgatcgaagcggaaggcaacatcgtcgagggcgctaaacgcgagttctcggcgggctccacgcggtcgaagaccctgagcatgcacagaagctctctgcacaggaactgggagatctcgccagtgcccaccgagcagctggcggtgggcgggtcggggaaggccgatggccgtacgcccgacatcgacgctggcgagaagtcagatgcttgcgtagtaccgaggaacgatccgaacaacgacgcggccgcaaagccggcgtctgcggaggatcgggagggaaggcgagcagccaagaggaacgccgaacaatctcccgcgccccggacacagagccggtcccgcgcgtcgacggggttgaacggcgtacgcgaagcagcctgtgcccaaaaggcatcgggcaagaaggtgcagttcaccgcgctgatgcaccacatcacgccgcgattgctgatcgacagcttcatgcatttgaagaagtcggcggcggccggggttgatggagtgacatggcacgactacgaggaatgcttggtcgagcggataggcaagctctgggacgcggtgcaagctggccgctaccgtgccctgccatcaaggcgagtgtacatacccaaagcggacggcaaacagcgtccgctaggcatcgctgcgctggaggacaagatcgtccagcaagcggtggtgacagtgctcacaccaatctacgagtccgacttccttgggttttcctacgggttccggcccggacgtggccagcaccaagcgcttgatgcgctgtgggtggggctgcattggaagaaagtgaactgggtgctcgacgcagatattcgctcgttcttcgacacggtcgaccacgggtggatgatgcggttccttgagcaccgaattgcggataagcgcctgctgcggctcatccgcaagtggctgacggccggcgtcatcgagaacggggcgaagacggaaatacgggtaggtaccccacagggggcggtgatctcaccgctgctcgcgaacatctacctgcactacgtctttgacttatggctccagcgatggcgtcgccgcgacgctaagggtgacgtgatcgtcgtgcgatatgctgacgatagcgttgtgggcttcgaggccgaggctgatgcctcgcgattcctggaggccttaaaggcacgatttgctcagttcgggctgtcgctcaacgaacagaagacgcgagtgttgcagtttggtcgctacgcggccagcctacgcaagcgcgctggcttgggccggccgcagacgtttgactttcttgggttcacgcacatttgcgcgaccaagcggtcgaatggcggtttcatcgtcaggcggttgacatcgagcaagcggatgcgtgccacactcaaggcactgcggcaggcgctgtaccggcgccggcacgaaccgatcgcagtggtcggcacatggctacgccgtgtgatgcagggctacttcaactatcacgccgtcccgggcaacaacctgcgattaagtagattccgttctggggtctgccgagcctggttacatgccctccggcgacgtgggcaatatgggcgaatgtcctgggcgcgattcctccgacgggtcgcgccatatgtaccgtcagttcgcgttctacacccttacccaacacagcgcttcttcgcgtcatgactcaaggcaggagccgtatgcggtagccctgcacgtacggatctgtgcggggggcggggggtaacctccgtccctaccgcgatccccgtcaca

SEQ ID NO: 155 - DNA, 1049 bp, Clostridium tetani
  source 1..1049: mol_type = genomic DNA
gaatatactatagccatacaataaaagtgcgaaacgttatcctataagtaagaaagttttaaaattttcttacgaaaaggatagaacttaaaagttctaactgttctactaaagtaataagtgaaaatcttatttaaagcaaacaaccaagtagctttaagtctaagtcccctacacaagttttatactactatgcaaaacttgtgaagctaggtaaggtcgtaatccgtgaaagtcggatgcggggctccttaaaagattactatggtaaacataagctaatccattaagatgcgatttatatgtattttatactgttaaatatttttgtgcttgtggcttggtataaaacagttaagatgaagtacttaactggttttggaataattggttgttaaactaaaacattataaatcgttagtggatacctaaggtaatcaaaaatagggataggtagaatggaacgtttgatgctgtatatgaagaggtttagtagaacctaggacacatatacgggctcagcaggttcatagtagctatgatactcagccggaagtcaaattaattttgaaatacttctatggtaacataggagaaggataaaactgagtgagccaaggaacctagtcggtaatagaaaagtggaagttaaaacaaatataagattttagaattaatttaattaatgaacggaattaatttaatgatatttaaagttagacggttataaattaaacatttcaaaattaaaccatatccaaattcataaatatagctagatcatatcactagtttaaaaataaataaatcatttcaaattactattaagtaaggtattaataccttacttaatagtaatctcattacataagagaattactagattagcagacagattcataaaaactatatcaactaggacaatagaaaatatatttatacacttcctattatcgagcgaacgccttatgcgatgaaagtcgcacgtagggtgtagaccaagcgaaatcctatgcatttaggatagtgaggtatagcaaaggagaaaa

SEQ ID NO: 156 - DNA, 866 bp, Thermosynechococcus elongatus
  source 1..866: mol_type = genomic DNA
aggttcttctgttgcgacgtggagctaaccagatgattgtctcgctaacccgaaaggttagaatgagaccagctgttcccccagctgtagcctagggaggcatgcgtgactggcaacggtcaggtatgaagccctcccgacaacgtagcctgaaccgtaaggttgaagccgaatccgtgaggaggaagcaacttcaccagtgtcaggtgatagggagctaggcttgagggtatggtgaacgtaagtgaagtgacgctagaagcctcgtgatcttagacaggccaaagacgctgataggcctgggccaaaatggcaaacggacaggactcactgtttgttcttcagtgaggggggacgatagctccgtcggggcagagtcaccacctaacccctcatatgtcatctaaacggaacgtggtaagcccgtactttcgccttgataggctaagaacaaggcaagtggaccgcggggaacacccatggaagtgcgggtaaaggaggtgggaaaaagcgaatgccaatctgtaatggattggatagggattgaggatggtggcaggacatccaacatcatcccactcgaaagagggcagacttcccactggtccccagttacgagaaaactcacagaacctccctaatggtgacaaagcaaatggcggtgatcttgagtcactggtgcggtcaccaacctgctcaaaaggtagagaggctgtagatgagtatcgtaaaggttgcatgcacaaccggttcctcttaaatgaggggtcctggggggctcgagccggatgcagggaaacttgcacgtccggttcctagggggctagggggcagcaatgcccccctgctacccgaccgacatttct

SEQ ID NO: 157 - DNA, 1930 bp, Azotobacter vinelandii
  source 1..1930: mol_type = genomic DNA
tgaacgcttagtgcgccagttaaagctggcggaggatagtcgatgaaagttcgatactggtaagaaatggcgaatcaaccagcccccgagtcatgcacgttgcaccgtgaggtgcagggtgaagcgttgacaggggaaaccgatgggccagccattgagccgcgtaatcagaaatccgggacgccgatgctgttaagcgaagcagaaggcaatacggggcatggcgttatacgccagtcatgccctggtcctgcgcggtcggagaccctgcgcacgtcgggaagtccttcgcacagaaactgggagatctcagtgatgcccggtgcgcatgcactgggcggagcaggcaaggccaaaagctgtaaccctgctgtctacgtcgctgagaagtcggatacgtccgtagtacctgagaaaccgtcgaacaaaggggctagccctgcggagagggtggaggaaaggggcgtagccaaggggaacaccgacaagaaccccacgccccggacgctgagccggagcagttgcgtgtcgatgggacttgaaggtgtacgtgaagcagcccgtagggacaagggtatgcagttcacggcattgttgcaccacatcacaccgcaactgttggcgcagagcttctacgcacttcgccgcgacgcggcggtgggagtggacggtatgtcgtggcgagaatacgaagaagacctccaccagagggtgggcaagttgcacgcacggctccacagaggagcctatcgggcaacgccgtcacggcgggtatatatccccaaagctgatggcaggcagcggccactgggtattgcctctttggaggacaagatcgtacagcaggcagtcgtcaccgttctgaatgcgatctatgaggaggatttccaggggttctcgtatgggtttcggccgggacgaagccagcacgatgcgctggatgcgttgacggtcgcgctcaagagccagaaggtgaactggatactggacgcggacatcacgtcgttctttgacgagatcgaccatgaatggatgctgatgtttctggggcaccgaattgcagaccgacgcatgctcggactcatctgcaaatggcttcaggcgggagtaatggaggatggccgtaggttggctgcgaccaaggggactccccaaggcgcagtgatatcgccgttgctggcgaatatctatcttcactacgtgctggatctgtgggcaaggcagtggcgccaacggcatgcccgtggcgagatgattgttgtgcgctacgcggacgacagcgtggtgggtttcaggacgcaatggcaggcgcagcgtttcctggtgcagttgcaggaacgcatggccaggttcggtctatctcttaatgcctcgaaaacacggctgatcgagttcggtcgctttgctgtacagaatcgcaggcggcaagggctgggcaaaccggagacgtttgatttcctgggcttcactcattgctgtagtaccaacagaagcgggggatttcaaatcctgcggctgacggtcaagaagcggatgcgtgcgacactgcaagccatccggatagcactgaatcgccggcgacatgagccgatccgagtcgtaggtcaatggctcggcagtgtggttggaggatatttcaactaccacgccgtgccgggaaacctgatccgtcttgacggttttcgggtggcggtttgccgtctttggcggcaagccctcaaacggcgcagccagcgtaaccggctccagtggtcgcgctatggacgccttgcagacctctatataccaagacccagaactgcacatccttaccctgaggagcgcttcgcgtcacgtacctgaggcaggagccgtatgcggtagttccgcacgtacggatctgtgcggggggtggcaggtaactgccattcctaccgcgacctataggaac

SEQ ID NO: 158 - DNA, 767 bp, Bacillus cereus
  source 1..767: mol_type = genomic DNA
gcaggtcgtggtgcgaatcgttcctaagtgaaaagcttaggcatcttagacaagggaaggccccattaagatagaactaatgattcgagagacagaatgatagagtaacgtcttgaaatgtttcccctaagtctccgacatgcattgaaaggtaggatgtttaaatgtgtgaagctcggtgaagacggctgacagataccgtagtgaaaaatgtgttttaaccgaaagtccttttagtaagaggatgtatcccataggccgggggtccgtaaagtatctatggtgagaatgttatatgaaataactgacgaactttcgaattaacgagtctataaccatacaatcagaaatgatagtaaaccatattttgtgttgtgtgtgaggttaagtaaattcgcggctatgaacaaccttacagtattataggtactgtctagcacgcaggctcatagaaggcacctaagggtaccatatagtggatagaatcattggaacgatgaaagctcaggacgcagagaacctacattctgtgaagcggtggtaaggaaaaggaggaatcctataacttatctttatttgagtgatagcgatgtcatagtagcgtggaatttatggaaacataaaggagcgaagggcattagtcaatattgtgaggaaaaagcaatattgatggcaagccgtatgaagggaaactttcatgtacggtttagtgtgggggaaaaagcagagattatatcaaagttttacctatcacaataaggagagaa

SEQ ID NO: 159 - DNA, 1933 bp, unidentified
  source 1..1933: mol_type = genomic DNA; note = unknown bacterium
gggtcgggccgaaagcgccgattaaaatcggtattggatagagaatgaaagagtcttatggtggaaggtttagcaaaccacaccagcctcgagtcatgcggcaattgcagtaatgcattggctgaagcgttgacagagggaagcacaggcgggctattgagctccgaaatcactcattctcgggtgccgaccacgtggtccgatggggaaggcaacacgttttgccgcgttattatgcgagctgtaagacgacccggcggagtcctagaaccttgcatgtgtggacatcctttacgcgagaatcgggagatccgtgagtcttcctcaaaatgaaaggaacggccagtgaaagagaaattctgtacgcctggcatgaacgacttacggaagtcggacaacaacatagtacctgagaagcttgcgaacaaggattttaaacatccgcggagcaggtggagggaagggcgttgaccaaggggaacatcataaacgctgccgcagtctgtacacagggacaggtagctgcgtcgaccggcctttgtggtgtgtgtagaaaagctcagcaggacaagagcgccagatttaacaatctttttcaccatattacgccagagctgttgacttcaagtttctacaacctgaatagaaaagcagctgctggagtagatgaaatgacatggcgcaagtacaaggaaggctcgcctggacgaatagcagatttaaatgaacgtgtgcataccggaagctatagagctaaacccgttagaagaagctacattaataaatctgacggccggaaaaggccacttggtgttactgctctggaggataagatagtccagcaggcagtctcaacgatattaaatcaaatctatgagaccgattttatgggtttcagctatggtttcagagaaaaacgtagtcaacacaatgccctcgatgctttatatattggaatatcgaggcgtaagataaattacatacttgatgcagatatctctggattttttgataaaatcaatcatgactggttattgaaattcctggagcatcgagtagcggatcgcaaaatattgcggctgattaaaaaatggctaaaagtcggtgtgattgaagatggtaaacgaacatcacttgaagttggtaccccacaagggtctgtgatttcacccgtgttggcaaatgtttacctgcactacgcacaagatctttgggcacaccaatggagaaaaaggcatgcagacggagacgtgattattgtgcgttacgccgatgatagtgttgtagggtttcaatacagaaaagatgcagacaggttcttgaaggatctgattgagagaatgggtcagttcggtctcgaactacacccagtcaaaactcgattaatagaatttggacgctttgcagtggttaatcgccggaaaagaggcgaaagaaaacctgaaacgtttgatttcttgggcttcacacactcctgctccaaaacacgcactggacgattcttcatacgccgtaaaacgattaagaaacggtttgtccagaagtataaggaagtgaagcaagaactgaaggaacgtatgcatgatgatgtgaaggaaactggaaaatggctgaattcagtcatcaggggatttcaaaattactatgcagtccctggtaatatgaatttggtaaagagtttctatgaccaaacaacgcgggcatggctacataatctaagacgcagaagccacaaagggcaaagtttcagttggttgcgctttacaaaattaatcaggtggttgataccgagagttcgaattgcacatccttttcctgaccagagatttcagcgttattacctgaggtaggagcccagtgcggtagttccgcacgctgggatctgaacggggggtgctgggtaaccggcattcctaccgtgacgtatcaagtg

SEQ ID NO: 160 - DNA, 1961 bp, Methanosarcinales archaeon
  source 1..1961: mol_type = genomic DNA
aaaatgataggagtgccgagataaatgcataatatccagttggtgaaaatccaacctgaggaaaggttagccgccagctcagaactgaaccttgcgcccagcctggtaacaggctgtggtgaagccaggggaatgggatactgggccgcaacgcaagtgaaggaattcagccacgaaatatttattgtcccggaggccgacgttgtcttgttgacggaaggcagtactctcatcgccgatagtcttattgggacatggcaaggcgatgcgagcccggcggggtctaagaccgtggcacggtatcaaacggatattatggaaactcgggagacccagagtgttctccttacaggagtatgtcctatcaagcctataaacggtaagactgtacagacgacactctgggagtcggaccagttcatagtaccggtgatgtggggtaatgcccatagagggaagggactggcaggagcgcgatggaccggcagggacacatcccccatacccagagatgggcaaggggtgtcaacaaaactgtggtccataactcaacgagccagagaagatccaacctgtaagtttacgtctttagcgcacttacttacggcggattttctcaaggaatgtttccgggagttaaagaaagataaagctcccggcgttgatggggtaacatggagaaagtatgaagagaacttagacgagaatacagaagatttggtaacaagattgatagcgaagcaataccgaccacaaccagtaaaacgagcctatataccgaagtcgaacggggaacgaagaccgctaggaattccagctctcgaagataagatcgtccagttggcgataaagaaaattctggaggctatctttgaagaggacttctgtgacgtatcctacgggtttcgacccaaccgcagttgccatgacgcactggacatggttgacatgatcatcatgaccaagccggttagctatgtggtggatatggacattgcgaagttctttgatacggtggatcacgaatgcctgatggaatgcctgaaacagcgagtagtagacccaagtctgctgcgaataatcgcccggtgcttaaaatcgggagtgatggaggaaggaaaatatctggagacggataaaggcactccgcagggagggatattaagtccgatacttgccaacatatacctccattatgcgttggacctttggtttgagaaagaggtgaaggagcagctaaaggggtttgctcagctcatccgttacgcagatgacttcattgtctgttttcaacacgatgatgaagccagagcgtttgggaaaacattaagggaaagactggctaaattcggactgacgatttcggaagagaagagcagaatcattaagtttggtcgctatgcctgtcagcaagcaagaaagcagagtaagaaatgtgcaacttttgacttccttggttttacgctctactgcgataaaacccgaaacggtaaatttaaagtggggcgaaagacatctagcaagaagttcaggcagaagatgaaaataatgaatctgtggctaaagggcgttcgaaatcgtgtgaaattagagttatggtggctattgttagcacaaaagatgattggacactaccagtactatggtataagtggcaatatacggggattacggagtttctactatcatgcgacggaactcgcctttaagtggataaacaggcgaagccagaggaagagctataattggagtcaattcaatcgttttctttcttttaatccactgccaaaaccgaagatatatcatttctatgccctatcaaaacaaaagtagaggatgtactcctgaagagccggatgagggaaaacttcaagtccggttctgtgagggggctcatagtaaccttggagccaatactccaacagaaggtggactatgagccctactcgacatacagaccc

SEQ ID NO: 161 - DNA, 2173 bp, Methanosarcina acetivorans
  source 1..2173: mol_type = genomic DNA
ggacggaatcgtgcgacgagaagttatgccatggattgcggtacaatccgcctttctatccattcagaatgtctctactctgatgtgcatctctggcaacggatttgtgcaaagctcagaggacaacgaggtgtaaaactgaacctgacagttccaaaccgctagggaaatggacatatggagaaccgaaaggtgaagattcacaggggtcgtgacttgcaacatgcgaaagcaggttgaattacgcatgagccaaatggcatcgaaacccggttatgacaatccgattccgtgtcataacgaatggacgaatgggtttccggcctataggatcctcctaagtgttcagcaactatctatggtatagaactcggtaaaccatttgtggccctataaggtaggaattccgtgaggaaaaccaataccacaggtggcagaggaaatggcaaaaagctaaagattccctgtaacggggaatatagaggagggaaaaaaaaactctttccttaatgcgaaagcatgcccacttgccattggtcacgaaagcaacagggattgttaattatgaatgtaagaaaaccagagataacttcggttacggaccttacagacaaagaactcacccaacaatggaaaagcattgattggaaaagagttaaagaagttgttaataaccttcagtctcgaattgcaagtgcagctaagaacggaaaatggataaccgtgaacaaactctcccgtcttctgacccggtccttatatgccaaactactttcagttcgtaaagtaaccactaacaagggaagccgaactcccggaattgatggaattatctggtcatcgtcggcagataagatgcgttccgctttacaactaacgaacaagggctaccgtgcaaaaccattaacacgaaagtacattcgaaagaagaacggtaaactacgacctcttagcataccaactatgtatgacagagcaatgcaaaccctgcactctctggtgctaggtccaatcgaatctgctataggtgacaagacttcgtttgggtttaaaccttaccgctcaactaaagatgcttacgcctaccttcacatctgtttaagcaagaaaattgctcctgaatggattgtcgaaggtgatattaaagcctgctttgatgaaatcaaccacacttggatacttgacaacatccctatggataaacgaatccttaaggagtttctaaaagccggatatgtcgagaattatcatctgtttcctacggaaaaaggcacaccacaaggaagccccatatctccaataattggaaatatggccttaaacggcttagaaaacgccttagcaatgagattttactccagatcagatggaacaattgacaaatctcatcaaaacaggcacaaagtcaattgtgcccgttttgctgatgactttgtggcaactgctgattccccggaaacagctcttgaaataatcgatgtcatccaagaatttttagatcctcgtggacttaagctcagcgaagaaaagactcttgtaactaatattagtgaaggattcaattttttaggctggaacttcaggaaatacaaaggaaaactccttccgaagccatctaaagactcccaaagggaaattatcaagaaaatcagtgatgtaattcacaaagcaaaagcatgggatcaagaccgactgatacgaatcctcaacccaatcattaggggatggacacagtatcacaaccatacagtttcctctgagattttcagcaaacttgatgatacagtctataacatgcttatctcttgggcgaaaagaagacactcaaataaaggtctcacctggataatgaccaaatactggcataaatccggtagcagaaaatatgtattctgcacagagctaaagacgttggagaggttctccaattccaaagttgttaggcaaagactagcaagccttaacaaaaatccatttatcgacaaggaatatttcgaacaatggaaattcatggaataccaccgaaagaaacgcatcaccaacccaattctgttctaaactgacacccgaaagggtagtagtggctcgagccggatgatgggaaactatcaagtccggttcctagaagacggggtagggagtaatcccaccctgttattcgacgtacgcgcca

SEQ ID NO: 162 - DNA, 2126 bp, Methanosarcinales archaeon
  source 1..2126: mol_type = genomic DNA
tggtctctgtttgttgacaagaagttgcatcacagattgcccaactggctaccccttccattcggggtaatcctaccgcggaatgcatggatggcaacgcccacgcagtaagcctgcggaacaacgtggaaaatcagaaaatccgattcggatcaactgctaggatacgaatgctatgaagagtgtaatgcgaaccatcgaaagagtcgtaacacaggataagcgaaagcggactgttacgcttagaccaaaaggtacgaaatcagactataactgctgcagagacgttatagagggtggacaaacggtttccggcttacagatggcttctaaggcgttcataattatctgtgacgtagaacttggtaagccccctaagctcccggaatatctgggtagagaccccgtgagggcgaacgatggcatagggggtagaggaatcgataaaaagcgaatgacatcttgtaatgaggtgcatagaggttcaaaatttgccttaacccaaaagggtgctgacttatcgagtggtgtctcattgcatgaacggaggcaaacctgtgaatgtaagtaattcaattacgcccttaaaaggcgagaagcttacagacaagcgaccaaaaccccggtgggacgataccgactggaagagggtcgaagaacatgttaacaggctgcaaaccagaattgcaaaagcagttaagcaagacaagtggaatttggtcaaaaggttaagatttctgctaactaactcgttttatgcgaaattgttggcagtnaagcgtgtaacccagaatagagggaagagaacagctggaatcgatggcgtgaagtgggcaacactgaactccaaaatgaacgctgctcttatattatccgatgtgaagtataaagcaaagccgttaagaagagtttacatctcaaagcagggaacgacgaagaagaggcctttaggaatcccaaccatgtacgatagggcgatgcaagcgctgtacgcccttgcgttactaccgattgcagagacgacatctgatccacgttcgttcggcttcaggatacatagaagcacacaagatgtgcgccaatatgcatattgttgccttggtgggaaatactctgcaaaatgggttttggaaggggatatcaagggatgtttcgataatatcgaccatgactggcttctgaacaacatcccgatggataaatcgattctaaagcaattccttaaggctggatttgtgtataatcgacatttgaatcctacccccgcaggaacacctcagggaggaattatctccccgatactggcgaacatgacactggatgggatggaaaaagctatctcatctgtgtattacgtcggtaagaatgggaaaatcgataaacatcgatataatctccacaaggtgaatttcgtgagatatgcggacgatttcatcgttaccgcaaactcagaagagacggcaaaggagattgcggagttgattaaagagttcctgaaggcacgaggtctggaattgtcagaggaaaagactcatatcacccatatcgattgtggctttgactttttgggctggaacttccgcaaatacggagggaagcttctgataaagccatccaagaactcgatggggaatctcatccgtaaaatcggtgatgtgatcaagcgagcgaaggcatggaaacaggaagacctcatcaacgtattgaaccccctcattactggctggtcgaattatcatcgatcggctgtagctaaggagatattcagcaaattagatcatattgtctgggatatgctctggaggtgggctaagaggagacacccggacaaacgtaatacgtgggttgctaatagatattggcattctgtgggaactcggaacagggtgttttctaccggaaggaataggttgaaactattctcggatacgaagattgtccggtgtgctggcttgaaattggataagaacccctttattgaccaagactactttaacttacggaactgctgcccgatactgaaagggttatgagatgcttgagcggtatgaggtgaaagtctcacgtaccgttctgagatgaggaggggcgggtaaccgccctattcttaatctgcggctatcctt

SEQ ID NO: 163 - DNA, 1895 bp, Methanosarcina acetivorans
  source 1..1895: mol_type = genomic DNA
gctgatgactgtgcgccacgaaagcgaatccgtgtgacagaatgtcgcatagtgtaactatgctgcatagaagatgagggtgggcccctcggaatcgctatacaggtagagattccaaaccaccataagctgctgtggtcaaaagccatggtagtgagcgttatggaaaaggcgagacttgacctcgtcaggtatgtgctatggagacgaacgcaatgaaccactgataaagtgtcgaaagcgtagggatgtcatcaaaaccagggggtagtcgttaacctgggataagtctggaagaaacctgtttactgaccagatggtggccggcatgaaggtggcgtgaacttagcacaggcttctgtgtggaacgtgggaacctgtacttcgatggtaagagaaaattccaagcggaggacccgtaaggaggaaagtatcgaagcgatgtacaggggcggaacaatccgtagtagtgatgaagctcctgtaatgggagtgaagcgaagggattgtgttatccagttttgaaaattggtcaacctgacaaatcaagggaggaacccatggacgaaacaaagccttatgaaatctctaaagatatagtacaagaagcttttcagagagtaaaagcaaacaaaggtgctgctggtgttgatgatgaaaacattgcagcttttgaatcagatctaacaaacaatctctataagatttggaatagaatgtcctctggctgctatttccccccatcagtgaaagcaatcgaaatccctaaaaagagcggcggaactcgcattctgggaattcccacagtactcgacagggtagcacagatggttacaaaaatctatttggaaccgcaattagaaccactctttcaccccgactcttacggctatagacccggcaagtcagctgcagacgctcttgctgcaacacgtaagcgttgttggagatataactggttactggaattcgatatcaaaggattgtttgacaatattaatcacgatctgctaatgaagcaagtcagtatgcatactgacaaaccatggatcattctctacattcagagatggctcaaagcaccctttcagatggcagatggaacagtcaatgaacggacaaaaggaactccccagggaggcgttgttagtccgcttcttgcgaatctgtttcttcattatgcttttgaccagtggatggatagtcatcatcggtataatccatttgaaagatacgccgatgatagtgtcatacactgccgaagcaagaaagatgcagatcgactgaggattaagttagaaaagcgtttgtctgaatttggacttgaactacatccaactaaaacacggatcgtctactgtaaagatgacgatcggcaagaggattatcctgaaacaaaatttgattttctcggatacacctttagaccccgtaggtccaaaaacaagtatggaaaacactttatcaacttcactcctgcagtcagtaatactgctaaaaagtctatgcagcaagaaatccataattggcgtatgcacctcaaacccgatttaacgttagaagacctatcccatatgtataatcctatagttagaggctgggtcaactactatggtctcttttacaaatctgaactgtactgtgtactcaagcatatgaatcgtgccttgactcgctgggccttgcgcaaatacaagaaactttcagggcacaagcgacgagcaagatactggcttgggaaaattgccagaagggatccaaaactttttgttcactggcaaatggggatttttcctgaggctggataatgggagccggatgagctgagaggttcaagtccggtgctgagagggcctgggggtgaaactcccccgggctactcaccttattgttac

SEQ ID NO: 164 - DNA, 847 bp, Bacillus cereus
  source 1..847: mol_type = genomic DNA
gtgcgacacgtttctttataagtgtgcaaacacgaagtaggagggttatcaatttgatgacaacaacggattgactgcaagtaggaatgaaagccgtcaggttgagctgaaactacttatctgatactcctatatgcaaggcgtgatagtagtcacaaattgtatgaagctaggtgaagtcggctgaacaaaacctaagtgagaaatcatatggtaatggataggtcgggatgctacaaaacatctatggtgagaatgtcctaacggactggcgaatgtacaggtttaaaggattaattcattagaaatgtgtatattgtcaacgagtagttatttcgacgctatctaccgaaaagtaagagtaaataatatgaaattcggaagatctaacgatgaggatgtaaagataacaggcttacagcaagcacctaaagatatatgtatagctaagtcattcagaacgtggtaagcaagagactgtcacaaatgcctactaacagacaaggtgcatataaggttctaacgaaccaaaattgctttattcttgtgaaggtggggacacagtaccgacgaagcatgtaacaaatgtggagggatagtccctagtcttgttcgttgaaaactaaatcaactggatataactcacaggatcgagtaagatgatgtgacttttcgtaagaaaagggaatgaatacgttggtgagtacaacatggtttgtaatctagttgttttagtataaaaggataagatggggcgctgtatgcgatgaaagtcgcacgtacagtgtcaagcgggggaaaagatggagataactttaaagtcttacctatcgcaacggataaaaag

SEQ ID NO: 165 - DNA, 969 bp, Bacillus thuringiensis
  source 1..969: mol_type = genomic DNA
cctatcactagcgcgacacgttcctaagtgaaaagcttaggcactgtcgaactcaacagttcagcagtgaactgtcattctaagaagtcaaatgaaggagtaacgtctggaagggcttcccttaatcctccgacatgcaggaaagtaggcaagtactgaactgtgtgaagctcggtgaagtcggttgaaggttaccgtaaattagtatctctaatacgaaagctatccagcggtggatggtgtaactgatagaccggaggtctataaaacactcaaggttaggatgcgcgatgaactagaggcgatcgctagtaagcgcagacgaatccctgatggtacgggtctatatcgggagggaatcgaaaggttctctgacacaaataagtgtcgctactgtgggtgagtaaaactctcctttatgaaagcccatatatcgttacaggcgttattaaggtagcaggctcataggggaaacctaaaagtgtatgtacagataagaatgacggaacgtggtaagctgccgacatggagggcttgttctctttgaagtgttgccaaggaaagtcacaatgagattagttgtcgatataacttggtttaacggcagtgaaagtggtggcacagtaccgatgaaacgtgtaatgaacgtggagggatagccactagtcgattgaagattgaaggttactattggttaacatggtttcgagtaagactaagagatgtaatgctccaaagtaataaggaggttacagcccatgttaaagaaaaccaagctaagacataacgaatattatgatacacaaaaaaagtgtatgacaatttatactcgaacagtcttaacggtaacaatttctttcaattggaaacgatggaacgccgtatgcccggaaacgggcgcgtacggtgtggagtgggggaaaagctggagataatctcaaaggcttacctatcactatcgcgacacgt

SEQ ID NO: 166 - DNA, 755 bp, Onion yellows phytoplasma
  source 1..755: mol_type = genomic DNA
tattgcaaaagggcgagaaagatgcctaatttcttagtaaattaaccacttacaactgcaattagtaatgcagtacaacatatttttggtctattttgtgaaagcaagatagattatagcagaaaataaaagcaaaaaaacgagaaactgtttatcgtttggatttgcgagttgaaaatagtcatggaaaagctttgcaataagcctaagagaaaaaaacgggcagtagctataaggaaaaacagttgataccttatattttagggcagtttatatattttttgcgtatataaaatttactaacacttattgatttaagtggcaagcaaaattgctaaacctaaaaccaccacaaccacataatttttttatttttgtatattaattgtggttatctctcctaaaactattaaggtaagaaattagagtaacttctatcttcctaaaagttatttgctaaaagaaagttatttgataaaagtataaaagcatctaaaataaacttaagtgtttaggctcaaggaaaaagcagaatcgtagtagtcgtaagaaattcaaagcttagctggaatgcataagtggactgacttaccaaggaatttgggaaaaccaaatacaaggcgaaagaagaaatatattttatataaacgctgtatgcttggaaacttgcttgtacggtgttgtgcgaggctcaaaaaaacctaattattagctaataattaaggcgttttgtttctatcgcgataattactcaa

SEQ ID NO: 167 - DNA, 865 bp, Thermosynechococcus elongatus
  source 1..865: mol_type = genomic DNA
agacattagggtgcgacgcgaaagctagccagatgattgtcccactagcccaacaagctagaacgggaccggttgtgcccccaaccgtagcctagggaggcatgcgtgactggtaacggtcaggtatgaagccctcccgacaacgtagcccgaaccgcaaggttgaagccgaatccgtgaggaggaagcaacttcaccagcgtcaggtgatagggagctaggcttgagggtatggtgaacgtaagtgaagtgacgccagaagcctcgttactctcagcaggccaaagacgctgacaggcctgggccaaaatggcaagtggacggctacacctctcctaattaggtgtacggggacattagctccaccggggtaaagtcaccacctaacccctcgcgtcatctggttggaacgcggtaagcccgtacttccgccttgataggccaagcacaaggcaagtggactgcgaggaacacccatggaagtgcgggtagaggagacgggaaaaagcgaatgctagcctgtaatgggttagatagggattgagaatgctggcaggacattcaacatcatcccactcgaaagagggcagacttcccgtcggttccccattacgagaaaatctatagaacctccctaatggtgacaaagcaaatggcggtgaccttgagtcactggtgcggtcaccaaccctcccaccaagggtaggcaggctgtagatgagtatcgtaaaggttgtacacacaaccggttcctcttaaatgaggggttttggggggcctgagccggatgcgaggaaacttgcacgtccggttcttagggggctagggggcagtaatgcccccctgctacccgactggccttgcg

SEQ ID NO: 168 - DNA, 859 bp, Thermosynechococcus elongatus
  source 1..859: mol_type = genomic DNA
tgacaaagcagtgcgacgcgaaagctagccagatgattgtcccactagcccaacaagctagaacgggaccggttgttcccccaaccgtagcctaaggaggcatgcgtgactggtaacggtcaggtatgaagccctcccgacaatgaagcccgaaccggaaggttggagccgaatccgtgaggaggaagcaacttcaccagcgtcaggtgatagggagctaggcttgagggtatggtgaacgcaagtgaagtgacgctagaagcctcgttactccaagcaggccaaagatgctgataggcctgagccaaaatggcaaagcggattggattcactgcttgcttttcagtgaacggggacagcaactccgccggggtatagtcaccacctaacccctcgtgtcatctggttggaacgcggtaagcccgtatcttcgccttgaacattcaaggcaggcaaaccgtaaggaatgctgatgggggtgcgggtagaggaggtgggaaaaagcgaatgctagcctgtaatgggctagatagggattgagaatgctggcaggacattcaacatcatcccactcgaaagagggcagacttcccgccggtccccccttacgagaaagtctatagaaccttcctaatggtgacaatgcaaatggcggtgatcttgagtcactggtgcggtcaccaacctgctcaaaaggtagagaggctgtagatgagtatcgtaaaggttgcatgcacaaccggttcctcttaaatgaggggtcctggggggctcgagccggatgcggggaaacttgcacgtccggttcctagggggctagggggcagcgatgcccccctgctacccgacaatggcggtg

SEQ ID NO: 169 - DNA, 858 bp, Thermosynechococcus elongatus
  source 1..858: mol_type = genomic DNA
gaggcgtggagtgcgacacgaagccgcttaagctgactgtcccactagcccaacaagctagaatgggaccggttattcccccagctgtagcctagggaggcatgcgtgactggcaacggtcaggtatgaagccctcccgacaacgtagcccgaaccgtaaggttggagctgaatccgtgaggaggaagcaacttcaccagcgtcaggtggtagggagctaggcttaagggtatggtgaacgcaagtgaagtgacgttagaagacccgttatcacatacaggccaaagacgctgacaggcctgagccaaaacggcaagcggacaagatacaacgcttccacgtcggtgtacggggatgttgatccgtcggggcatagtcaccacctaacccctcgtgtcaactaagcggaacgtggtaagcccgtatcttcgccttgaacattcaaggcaggcaaaccgcaaggaaagctgatgagggtgcgggtagaggaggcgggaaaaagcgaatgctagcctgtaatgggctagatagggattgagaatgttggcaggacattcaacatcatccgactcgaaagagggcagacttcccgctggtccccgattacgggaaaacctacagaacctccctaatggtgacaaagcaaatggcggtgaccttgagtcactggtgcggtcaccaaccttacacacaaggtagagaggctgtagatgagtatcgtaaaggttgtacatacaaccggttcctcctaaaagaggagtcttggggggctcgagccggatgcggggaaacttgcacgtccggttcctagggggctagggggcagtaatgcccccccgctacccgacaaaggctttc

SEQ ID NO: 170 - DNA, 2546 bp, Nicotiana tabacum
  source 1..2546: mol_type = genomic DNA
cggcttttaagtgcggctagtctcttttacacatatggatgaagtgagggattcgtccatactctcggtaaagtttggaagaccacgactgatcctgaaagggaatgaatggtaaaaatagcatgtcgtatcaacggaaagttctgagaatatttcattgttcctagatgggtataaaaccgtgttagaattcttggaacggaacaaaataaagttgggtcgaatgaataaatggatagggctgcggcttcaattaaattatagggaaagaaagaaaaagcaacgagcttttgttcttaatttgaatgattcccgatctaattagacgttaaaaatttattagtgcctgatgcgggaagggtttcttgtcccatgagtggattctccatttttttaatgaatcctaactattaccattttctattacggagatgtgtgtgtagaagaaacagtatattgataaagaaagttttttccgaagtcaaaagagcgattaggttgaaaaaataaaggatttctaaccatcttattatcctataacactataacatagaccaattaaacgaaacgaaaaaaaaaagagatgatagagaatccgttgagaagtttacctgtatccaaggtatctattcttactaaaatactttgttttaactgtatcgcactatgtatcatttgataaccctcaaaatcttccgtctttggttcaaatcgaatttcaaatggaagaaatccaaagatatttacagccagatagatcgcaacaacacaacttcctatatccacttatctttcaggagtatatttatgcacttgctcatgatcatggtttaaatagaaataggtcgattttgttggaaaatccaggttataacaataaattaagtttcctaattgtgaaacgtttaattactcgaatgtatcaacagaatcattttcttatttctactaatgattctaacaaaaattcatttttggggtgcaacaagagtttgtattctcaaatgatatcagagggatttgcgtttattgtggaaattccgttttctctacgattaatatcttctttatcttctttcgaaggcaaaaagatttttaaatcttataatttacgatcaattcattcaacatttccttttttagaggacaatttttcacatctaaattatgtattagatatactaataccctaccctgttcatctggaaatcttggttcaaactcttcgctattgggtaaaagatgcctcttctttacatttattacgattctttctccatgaattttggaatttgaatagtcttattacttcaaagaagcccggttactccttttcaaaaaaaaatcaaagattcttcttcttcttatataattcttatgtatatgaatgcgaatccactttcgtctttctacggaaccaatcttctcatttacgatcaacatcttttggagcccttcttgaacgaatatatttctatggaaaaatagaacgtcttgtagaagtctttgctaaggattttcaggttaccctatggttattcaaggatcctttcatgcattatgttaggtatcaaggaaaatccattctggcttcaaaagggacgtttcttttgatgaataaatggaaattttaccttgtcaatttttggcaatgtcattgttctctgtgctttcacacaggaaggatccatataaaccaattatccaatcattcccgtgactttatgggctatctttcaagtgtgcgactaaatccttcaatggtacgtagtcaaatgttagaaaattcatttctaatcaataatgcaattaagaagttcgatacccttgttccaattattcctttgattggatcattagctaaagcaaacttttgtaccgtattagggcatcccattagtaaaccggtttggtccgatttatcagattctgatattattgaccgatttgggcgtatatgcagaaatctttttcattattatagcggatcttccaaaaaaaagactttatatcgaataaagtatatacttcgactttcttgtgctagaactttagctcggaaacacaaaagtactgtacgcacttttttgaaaagatcgggctcggaattattggaagaattcttaacgtcggaagaacaagttctttctttgaccttcccacgagcttcttctagtttgtggggagtatatagaagtcggatttggtatttggatattttttgtatcaatgatctggcgaattatcaatgattcattcttagattttctaaatggaaatttgtttctaaatgatgaagagataaaaaaatttcactattctgaaatgttgattgtaatagtaattaaggggtaaatcaactgagtattcaactttttaaagtctttctaatttctataagaaaggaactgatgtatacatagggaaagccgtgtgcaatgaaaaatgcaagcacggcttggggaggggtctttacttgtttatttaatttaagattaacatttattttatttaacaaggaacttatctactccatccgactagtt

SEQ ID NO: 171 - DNA, 1545 bp, Euglena myxocylindracea
  source 1..1545: mol_type = genomic DNA
cttttaggtgttgtgttttaatcattatataagaatttacaaaaaaatttgtgactttacaaatcatattaaaacaaagttttaactgagttttttattttatcttttaataaactaatgttttggaataaatcgtttgtggtggttagtaatttgcaaaaaaaaattttcaagagtagttattccaatgattttaagcgttccaaaagctttcaggctttaattttaaaatcaaatgctgcaaaatacttagcaattcgctatgttctacaatctaatttggataaaaaaaatttaactattgatggcaaaactactttaagttatttagagcgtatctcgttattagagtttttaaaaattaattataataatcggtttcctcaaactatacaaaatgtgggttctttgaacaaaaatggtgattttaagttaacatttacttgtacgatatcagatcgtgtatggcaaacttttttaaatttttctatgcaaccggctcatgattctacttttcatccctttaattttggctttcggtttactacggaaaacttttttatcctaaaagtcttacttttaaatttaaaaaatcaatctcttcctttacaaaaacgtatatactatttttctttagaaaatagattggttgattttgattttctttatttagtgaaaaagttaagaatttttagaactattaaaattggaatctttcgtttattaaaaaaaaactactctttgcagtattgtgaagataagtatgatacattttctacattgttatttaatgttcttttggatggcttgctagattttggtaatatttttcattatggttatcattttttgttaattctcaaaccaatagagagcgagtttgttttttttaataatatttcgtgttttttttctagtataggtgtttctttttcagggaagtattcattactttctattacaggtttcgattttttatactggagctttacaaaaaaaggaaacttttatatttcagttccttccacaaaaaactactttaattttcttggacgagttaaatatatagttaacaactcgaattatggtgtaaaaatcaaagctgctaaattattcccgataatcaaaaattggtatgtttatcataaatattcttttaagaaaggttgtagctctattattttttctataaaaaagaaagtattaaaagcttttgttaaagagtctaaacaagatatttattctgctaagagattattggataaatgctttttttttgttgttattcacagtcttaaaattatcaagcaaaaaaaattttttcttgcaaaacacttagtttttcgcacaggttatttaaaatttcaatgtattcactgtggcgtgaatttaattttctcataatgtttctaaaattaataatgttttcccttaatttttctagtcaagattttatctatggcctaaaatataattttttcttaacaaaatctttcctttaattaattttgaaaatctcaacggattaatcttttctcatgtagc

SEQ ID NO: 172 - DNA, 2154 bp, Methanospirillum hungatei
  source 1..2154: mol_type = genomic DNA
agttaatatagtgcgacaagaagcagtaaatcggattgcgaacccggtttaaaatcggtaacgccacccaatccattctgggcgtccctacctgcacgtgcgtgtttagcaataaacgggtgcgaagctgcaggaacaacgtggtgattcccatagtctctttgggataagctgccagggaaaggtatcatggacaaacaaacgttgaaccgctgcaggatcgttaattatatcaggaggaaagagatttgcccctgtccccactgagggagaatcacggtatcagtagcttgaattatgctggtacgaatggacgaagctgattccggtctatgggcgggtcctaaaataccacaattatccgatttattgaacttggaaaacccaatacgctcctaaaaaaaaaaaaaaaaaatttttttttaggtaagaaccttgtgagaggaaccacatgcgtagtgggagtaggaggctgcaaaacgcgaatgcccggctgtaatggctgtggataggggttcaaactttgccctgacctgaaagggtgccgacgtgcagctggtttttcacggcgaacaggatggtttcaatgaacgggataagttcaattacgcataaaagcgaggacatctcggacagaaaacttgcaaaacaatggaagaaatttccatttgctaaagcaagagattatgtaaagcgacttcagacacgtatcgcaaaagcagtgaagaacggccaatatcgacttgcaagacgactccagtatctgcttacacattcgttttatgcaaaaatgttggcagtacaacgagtaaccaaaaacagaggtaaaagaagtgcaggagtagatggggaaaaatggaccacccctgaacagaaaatgaaagcagcattaacgctttcggacaagggctatcgggcaaaacctctccggagaatctacatccctaaaccgcaatcgagtaaaatgcgacctctttcgattccaaccatgtatgaccgtgctatgcaagctttgtatgcaatggctcttatgccttgggctgagaccacagcagacaagacatcattcggattccgaatgaagcgaaatgcacaggatgctgcttcatacacttttcagtgcttaagcagaaagacttcaggtcaatggatattagaaggtgatatccgcgggtgcttcgataatttcgcacaccaatggatgcttgataacatccctcttgaccaaagaatccttaaccaattcctgaaagccggttatatttatgatggaatactctaccgtaacaagtcaggtacgccccaaggcggcttaatttcccccttattggctaacatggctcttgacggcatggaaagaatgttgaaagaacactttcccggaaataaggttcatctcatacggtttgcagatgattttctcgtaacggcagactcacaggaaacggcactccagtgcaaggaactcatcactgaatttcttcatgaacgagggcttgaactctctgaggaaaagaccaaaatcgttcatatcaacgaggggttcgatttcctgggctggaatttcagaaaattcaaaggcaaattcctgatacaaccttcgaagaaagctattgccgcaatcattgataaagtaagggtaatcattaagtcggcgaaggcctggaaacaggaagaccttatcaaagccctaaatcccgtaattaagggatgggcaatgtatcaccggacggtttctgcaagtatgacctttgggaaacttgactgggttgtccgaaatatgttgtggagatgggcaaagcgccgtcataataataaagggaagagatggattgccagaaaatactggcacccaacacttaccagaaaacaggtctttagaacctctactcttactctcgaaaacttctccaacaccaaaattcaataccgaaaattcataaaactggatgcaaatccgtttattgataccgagtattttgaaaacaggccaggagttttcctctcaaagcaaagatcgatacggatgttccttcactacgcccataaaagcgggtagtgaaaaacttgagcggattgcggtgaaaatcgcacggtccgttcttggaaggctgggttagggtaacctaacctggctattcgacatacaccaaa

SEQ ID NO: 173 - DNA, 1858 bp, Methanosarcinales archaeon
  source 1..1858: mol_type = genomic DNA
gcggatgactgtgtgccacgaagctgcacgagagatgagggtttggcccctcgaagcctgcttgcaggagcaggcttcaaaccgccttaagctgctgtggttaaaagccatggtggtgagcgttaaggaaaaggtcccacaagatgggatcaggtgtggatcatggaaacgaacgtaagtgaaccgctgaggaagtgtcgaaagcgtatggacggcgtcaaaaccaggggctggtcgttgacctgggataagtctgggggaaaacctgcttactgcccagatggcgtccggcataaaggcggcgtgaacatggtcctggctctcgtgtggaacgtgggaacctatcccctaatgttaagggagaaacacaagtgggaggaccccacaagtgtgagagtaccgatgcaggggaataggggcggaccagcccgtattagtgatgaagtctctgtaatggagatagagcgaaggggttgggtcatcctgcttgatttaaagatcaaccaggagatgggaggaatctttgatgaaagcaaagccatttgagatttccaagaaagttgtattggaagcgtggaaagaggtgaaagcaaaccgtggagcagcaggtgtagataagaagtcgattgcggattttgagaaggatcataagaataatctttacaagatctggaatcggatgtcatcggggagctactttcctccaccagtgaggacggtagggataccaaagaagagtgggggagagaggttgttaggcatacccactgtggctgacagagtggcccagacagtggcaaagaggtaccttgagcctttggtagagccatactttcacaaggactcatacgggtacagacctggtaaatcagcgattcaagcagttggagtgacgcgtaagcgttgctggaggtatgattgggtgcttgaattcgacatcaaggggttatttgacaatattaatcatgacctcttgataagggcggtaaggaagcacacaaactgtaaatggatgcttttgtacatagacagatggttgaaggctccatttcagggagaggatggcacgctagtgcagagagaaaaaggaacaccccaagggggtgtgataagccccttgcttgccaatctcgttttgcattatgtgtttgataaatggatggagcgaaattattcgcaagttccattttgccgctatgctgatgacggagttgtgcactgtcggtctgaagcagaggctttaaggctaagggaaaatctggaggctcggtttgggaagtacaatctcgagcttcaccctgagaagacgaggatcgtctattgtaaagatgacactcgtcgagatgagtatcccaacacgagttttgattttcttggctttacattcaggccaaggaggtcaaagaatcggtggggtaagtatttcatcaacttcactcctgccgtgagtaataaagcggcaaaggtgatgaggcagaaggctcgaagatggaaaatacacctgcgaagtgacatgtccttagacgaactgtcaaggatgctcggtcccattatcaggggctggatcagttattacgggagcttttataaatccgcgctgtacccaattttgcgacatctgaacaggatattagttagatgggcaatgaggaaatttaagaggtttagacgccatcgtaggcgtgcggaatactggttgggggagattgcccaaaggcagccctggctatttccacactggcagatgggtgtgagacctatggctggatgatgggagcccggtgagctgagaggttcacgccgggttctgagaggacgtaggggggaagttcccctgcgttactcacctcattgtctg

SEQ ID NO: 174 - DNA, 1961 bp, Methanosarcinales archaeon
  source 1..1961: mol_type = genomic DNA
ataaatgtgggagtgccgagctaaatgcataatatccagttggtgaaaatccaacctgaggaaaggttagcagccacctcagaactgaaccttgcatccagtctagtaacaggctgtggtgaagccagggggatgggatactgggccgcaacgcaagtgaagggattcagccacgaaatatttattgttccggaggccgacgttgtcctgttgacggaaggcaatactctcgtcaccgctagtcttaataggacatggcgaggtgatgcgagcccggcggggtccgagaccgtggcacggtatcaaatggatattatgcgaactcgggaggcccagaatgttctcctttcaagagtatgtcctatcaaacctataaacggtaagatggtacagacaacactctgggagtcggaccagttcatagtaccggtgaagcagggtaatgccttcagagggaagggactggcaggagcgcgttggaccggcagggacacaccttccatactcagagatgggcgagaggtgtcaacaaaactgtggtccataactctacgagctagagaagatccaacctgtaagtttacgtctttagcgcacttacttacggcggattttctcaaggaatgtttccgggagttaaagaaagataaagctcctggcattgatggggtaacatggagaaagtatgaagagaacttagacgagaatatagacaatttggtaacaagattgatagcgaagcaatacaggccacaaccagtcaaacgagcctatataccaaagtcaaatggggaacgaagaccgctaggaattccatctctcgaagataagatcgtccagttggcgataaagagaattctggaggctatctttgaagaggtcttctgtgacgtatcatatgggtttcgacccaaccgcagttgccatgacgcgctggacatggttgacatgatcatcatgaccaagccggttaactctgtggtggatatggacattacgaagttctttgacatggtagatcacgaatgcatgatggaatgcctgaaacaacgtgtggtagacccaagtttgctgcgaataatcgcccggttcttaaagtcgggagtgatggaggaaggaaaatatcagaagactgatagaggcactccacagggagggatattgagtccgatacttgccaacatatacctccattatgcgttggacctttggtttgagaaagaggtgaagaagcagttaaatgggtttgctcagctcacccgttacgcagatgacttcattgtctgttttcaatacgatgatgaagccagagcgtttgggaaggcattaagggaaagactggctaaattcagactgacgatttcggaagagaagagcagaatcattgcgtttggtcgttatgcctgtcagcaagcaaggaagcagggtaagaaatgtgcaacttttgacttcctcggttttacgctctactgcgataaaacccgtaacggtaaatttaaagtggggcgaaagacatctagcaagaagttcaggcagaagatgaaaataatgaatctgtggctaaagggcgttcgaaatcgcatgaaattagagttatggtggccattgttagcacaaaagatgattggacattatcagtactacggtataagtggcaatatacggggattacagagtttctactatcatacgacggaattcgcctttaagtggataaaccggcgaagtcagaggaagagctataattggagtcaattcaatcgttttctctcttttaatccattgccaaaaccgaagatatatcatttctatgccctatccaaacaaaagtagaggatgtactcctgaagagccggatgagggaaaacttcatgtccggttctgtgagggggctcatagtaaccttggagccattactccaacagaaggtggactatgagccctactcgacgcatctgtgt

SEQ ID NO: 175 - DNA, 2126 bp, Methanosarcinales archaeon
  source 1..2126: mol_type = genomic DNA
gttctctgcctgttgacaagaagttgcatcacagattgcctaaccggctgccccctccattcggggtaatcctaccgcggaatgcgtggatggtaacgcccacgcagtaagcctgcggaacaacgtgaaaaatcagaaagtccgattcggatcaatcgccaggataaagaacgctatgaagagtgtaatgcgaaccatcgaaagagtcgtaacgcaggataagcgaaagcggactgttacgcttagaccaaaaggtacgaaatcagactataacggctgcagagacgttatagggaatggacagacggtttccggcttacaggtggcttctaaggcgttcataattatctgtgatgtagaacttggtaagcccaatatgctcccggaaaatccgggtagggatcccgtgagggcgaacgatggcatagtgggtagaggaatcgataaaaagcaaatgtcgatctgtaatggatcgaatagaggttcaaattttgccttaacccgaaagggtgctgacttatcgagtggtgtctcattgcatgaacggaggcaaacctgtgaatgtaagtaattcaattacgcccttaaaaggcgagagacttacagacaagcggccaaaacccctgtggaatgataccgactggaagagggtcgaagaacatgttaacaggctgcaaaccagaattgcaaaagcagttaagcaaggaaagtggaatttggtcaaaaggttaagacatctgctaactaactcgttttatgcgaaattgttggcagtaaagcgtgtaacccagaatagagggaagagaacagcgggaatcgatggcgtgaagtggacaacacagaactccaaaatgaacgctgctcttaaattatccgatacgaagtataaagcaaagccattaagaagagtctatatcccaaagcctggaacgacgaagaagcgacctttaggaatcccaaccatgtacgatagggcgatgcaagcgctgcacgctctcgcgttacaaccgattgcagaaacaacagcagatccacgctcgtttagcttcagggtgcatagaagcacacaagatgcgcgtcaatacgcattctgttgccttggtgggaaatacactgcaaaatgggtttgggaaggggacatcaagggctgctttgataatatcaaccatgactggcttctggacaatattccgatggataaattgattctaaagcaatttcttaaggcgggatttgtgtataatcgacatttgaaccccaccacagcgggaacaccacaaggaggaattatctccccgatactggcgaatatgacactggatgggatggaaaaagccatcgcatctgtgtaccaagttggtaagaatggcaaaatcgataagtgtcgatataatccccataaggtgaatttcgtgagatatgcggacgattttatcgtcaccgcagactcgaaagagacggcaaaggagattgctgagttgattaaagagttcctgaaggcacgaggtctggaattgtcagaggaaaagactcatatcacctatatcgattgtggctttgactttttgggctggaacttccgcaaatacggagaagagcttctgataaagccatccaagaagtcgattgggaatatcatctgtaaaatcggtgatgtgatcaagcgagcgaaggcatggaaacaggaagacctcatcaacgtattgaaccccatcattactggctggtcgaattatcatcgatcggctgtagctaaagagatattcagcaaattagatcatattgtctggaatatgctctggatgtgggctaagaggagacacccggacaaacgtaatacgtgggttgctaacagatattggcattctgtgggaactcggaactgggtgttctctaccggaaggaataggttgagactattctcggatacgaagattgtccggtgtgccggcttgaaattggataagaacccctttattgaccaagactactttaactttcggaaccgttgcccgatactgaaagggttatgagatgcttgagcggtatgaggtgaaagtctcacgtaccgttatgagatgagaaggggcgggtaaccgccctattcttaatctgcgtgctctgcg

SEQ ID NO: 176 - DNA, 644 bp, Amoebidium parasiticum
  source 1..644: mol_type = genomic DNA
taacagacgcatgaatgtcttggaatgaaaatggaaacccacgtattggattcaaacagggtgaagtaaactttcctttttatggcatgtgtatttagaacttatatctttaacatctagtttccctggcattcgacaatcaatagtaagaggaaagtctcataattcacactcttttgatacaaaaaatctccaatgcttaaatgaactaagcaatattctgtacactaatataaatggtaaaatggtaaaatctattaaaggagaactatttgattatttaaactacagaacattagcgtacatgataatgggagatggagcgcgcaaaaataaagggtttgtaatatgtctcgagaatttcacacttcaggaagtcgttttactattaaatatgttagcaatcaaatttaatttggaggacctaactattcaaactgaagactctaaaccgtctccttttaaaaaagaaaaagaggaaattaagaagatttatagaatctactttggaatagagggaaccaaaacattattacctttcattaaaccttatttactagaccattttgattatcgcatatcgggaatgactaaagcacagtatgaacaacgtggtgtaaagcataaatcttaggtagtaggag

SEQ ID NO: 177 - DNA, 680 bp, Mesostigma viride
  source 1..680: mol_type = genomic DNA
agaaaattggatgccgcgatttttttgcataagtgatccacatttttccctaaattcttctttatttctttccgtggtcactgcaatttctgcattatcagatcctttagatatttggatagaaaaatcgttcggaaatgaaaaatttttttattcacgttttatggatcaatgtattgtagcatgtttttcagacgctgattttttaaattcaaaatttgaaaaaatatcacaaaaatcttgttggaaattttctaaatgtttttccactgttccttttttgggtggcacgctaattcactcttcaacaaggaaacccattgaatctaatgagctaaaaagtattaataatattaacagttcttggaaatggaaaatttcatggaatgttcttataaggttttgtcaaaatgaaagatattttcatcaaaattctgcaaaacctacggctgtgccctaccttgttccacgaactcatgaagagattttggctcattataaaaaattaatttttcttttttatctttattacaatcctatgcaagatcaccaagcattcaattgtttagtagatggtttaaaggaatcatgtatattgacattggcccttaaatataaaatgaaaagtagaactaaaataattcaaaaactttttcctcaaaagctgtaggagtttttct

SEQ ID NO: 178 - DNA, 2817 bp, Azotobacter vinelandii
  source 1..2817: mol_type = genomic DNA
gcatgatgtagtgcgacgagagtcgctataggtaggagtacctagcgggctacatcctaggcggtcaccacatggttggtggccgcttttcccgcaaaacagctctaaaaacccgagatccgggcgacaaaagcccggattcagggtgccgtcaaggcgctaaggattcggcaattcgttgcctaaatatccggtcttccaccgtggagaccataaaacgatattcgtactgagcctgtatttcgcgttttgagtacaggggaggtatgggagatcgtgtccaaaagctggctgctcctatccgtagggcgcaagccatagctgtcattgtctcacccgaaaaccgcaaggtggccggggcgcgagaccttctgttcagccagaaagtagcctagggacagtgcgtcgccggtaacggcggggtgcgaagcgtcctgacaatgtagccccatgatgtctgtggtgcgtggtcgccgcgaggcgccatgcagaccctgcgagcaacacgcgggcgaggcgctaggctgggtggtacggccaacgtaagtgaactgctgataaacaccgtcaaccgcatggagccaaagttgctgacaggcttcgaccaaaagggtgcgtgactggttgtccctcttacatctggggacacgctgacatcgagtcaccggtgaacaggcagggcctaacccactcttgtgatggttatggaactcggtaagcccgtattttcgccactggctggtagagcgggggcaggtcgaccgcaaggaaggctgttgaaagtgcgggtaaaggaggtcggaaaaagcgaatgccgccctgtaacggggctggataggggccgttgccccgacccgaaagggtgcccacttccgactggtctttgatcgcgaaaaggcttgtaaagccataaaagctggcgtcgttcggtccgaccggcggcgtctgaacagaaatcctcgcggggtagtgaactcactccccgcaagggcgacgtgcgtccgctgctcctccggggaaatcttctttggaggaaagcaagatgagtgcgtctgaaaaggaagtacctgcgtcctccctcccggccagcagttggcacacgatcgactgggcaaccagtcatcggcgggtgagagggctacaggtacggatcgcgaaggcagccaagaatcggcaatggcggcaggtgaaaaccttgcagcgcatgctggtccgctcgttcgccgccaaggcattggcggtcaaacgggtcactgaaaaccggggtcgcaggactccgggcgtcgacggggaaacctggagcacgcctgaaagcaaatggaaggctatttttcggttacagcgcacgggctaccggcccaggccgttgcggcgggtgtacatccccaaagccaacggccagcgccgaccgctgggaataccgacgatgctggatcgggccatgcaggcattatacctgctggcgctcgaaccggtgtccgagaccacggcggaccggaattcctatggctttcgtcctcaccgttccacggcggatgcaatcgagcaattgttcgtcaacctcggtcgcaagcactccgcgcagtgggtcatggaaggggatatcaaaggctgtttcgacaacatcagtcacgattggctgatcgccaacgtcccgctggacaaggcggtcttacggaaatggctgaaggcgggatacctggaatccggccaattgaaccccaccggggcgggaactccgcaaggaggcatcatctcgccggtgctggccaacctggcgcttgatggtctggaaaaggcgctcgaatcgcgattcgggcaacgcaacaccaaagccagctacaagaccaaggtcaactatgtgcggtatgcggacgatttcgtcatcaccggcatctcgaaagagctgctggtgaatgaggtgaagcccgtcgtcgccgccttcatggcggagcgcgggttgagcctcgcggcggaaaagtcgctgttcacgcatgtatcggaggggttcgatttcctcggccagaatgttcgcaagtacggtgacaagctgctgatcaagccagctcaccggaacgtcaaggcatttctggccaaggtcaaggcgttgatcgaggggaacaagacggctccggccagtctgttgatagacaagctcaacccggtcatccggggctgggccaactatcatcggccgatagtcgccaagcagaccttcaattacgtggattaccggatatggaagttgttgtggcggtggtgcaggcggcgacatggaaatcgctgcaaacgttggatcaaggagaaatacttcaagcgcatcggtacgcgcagttgggtgttctccgggcggtatcccagcggcaaactggccacattgctgtacgccgacgacacaaccatacagcggcacaagaaaatcagagcggaggccaatccgtacgacccggaagacgagatgtacttcgaggaaaggctggaatacgcctggcggagatcggacgaagggaaacggaaaacgctgaggctgtggctaggccagagcaagcgctgcccgatgtgcaagcagttgatcacgttcgagacgggatggaacatccaccacatcatcaagcggcacatgggcggaggcgacgagctggacaatctggtgctgttgcatcccaactgtcatcgacagctacacagtgcggcaccggctctttcgattgagaaagggcttacaaaggcttgagccgtatgcggggaaactcgcacgtacggttcttaggggaggggtggccagcaatggtcgctccttacccgacagccctgtcg

SEQ ID NO: 179 - DNA, 1879 bp, Azotobacter vinelandii
  source 1..1879: mol_type = genomic DNA
ctttttgctggtgcgcccagcatgggcgcactcctgcgggtgcaagtcccgccatgagctggtcacagcgaatgaagcgaagcgcaactgcgtgagggcgaccgagcgtggggaggaagcgcggagcgtaaatcgtgagccgatgaacaagaaccgcatagaaggcgctgccgagcagggcaagcgggccagtaaccgcgaagctcttgtgaccaaggcgaggcggcgtagatgcggcggttgtgcggtgaaggagtgcgttcttacctggggagatctcgcctcatgcctgaaagggcgacggtgtcgagccggagcgagaagtcagcagaggtcgtagtagccgcagtagcggggccgcagaggccagatgcgaaccggtgaaggaccgaacgagaggaagtgttggaaacgatgtcgatgtcggaggccatgcaccagaagcccgcgtgcgcggggcggcatgccgcaggccagggtgaagccccggccaaggtatcccgtggtgaagccgaaggcccgcgacatgagatggaaggcacagggtcggcgctgctggaagcggcgctgacgcgagagaacctgcggcaagcgttcaagcgggtgcgagccaaccggggatcggcgggcgtggacggtctggacatcgaccagacggcgcgcaagctggtgaccgagtggcctgcgatccgggagcaattgctgcgggggacgtaccggcccagtccggtacggcgggtgatgattccgaagccggatgggagccaacgagaattgggtattccgacggtgacggatagactgatccagcaggcgttgttgcaagtgctgcaactgctgcttgatccgagcttcagtgagcacagctacgggttcaggcccggaagacgggcgcatgacgcggtgttggccgcgcaatcgttcgtgcagtcgggccggcggatagtggtggacgtggacctggagaaattcttcgaccgggtcaaccacgacattctgatcgaccgcctacgcaaacgcatcgacgacgcgggagtcatccggctgattcgtgcgtacctgagcgcggggatcatggatggcggggtggtcatcgagcgggaccaggggacgccgcaaggcgggccgctgtcgccgctgttggccaacgtcctgctcgacgaggtggaccgggcgctggagcggcggggccattgcttcgtgcgctacgccgatgactgcaacgtgtacgtacgcagtcggcgggcgggcgagcgggtgatgaatctgctgcgcaagctgtacggccggctcaagctgagggtcaacgaagccaagagcgcggtggccagtgcgttcggccgcaagttcctggggtatgccttctgggcagcgccgaagggacaggtcaagcgcaaagtggcggccaagccgttggcgacgttcaagcagcggatcaggcaactgacgcggcgcagcggtgggcgcagcatggcgcaagtcgtgcaggagctgcgtccgtatgtgctgggctggaaggcttacttcggactgtcgcagacaccgagagtctggcgttcgctgggcgaatggctgcggcatcggttgcgtgccgtccagctcaaacagtggaaacgcggcaagaccctgtttcgggaactgcgcgccctgggggccagccacgaggtggcgcaacggatcgcggccaacagccgccgatggtggcgcaacagcggcaagctcctgaacagcgtgctcaacctggcgtggttcgaccggcttggcctaccccgactcgcctgacctcaagctctcgaaccgcccggtgcggacccgcatgccgggtggtgtggcaggggcgctgccttgacggcagccccctatgccgatcatgccggaa

SEQ ID NO: 180 - DNA, 1879 bp, Agrobacterium radiobacter
  source 1..1879: mol_type = genomic DNA
ttggacgagggtgtgcggcgaggcacccgcagcagtgtgggagccgagctgcacgaaagatgagggtaggtccctcgaagccgaccgtcaggggtaggtttcaaaccactgcaagctgcgacggtaaagagccgtggtggtgagcgttgcagaaaaggcggatgttagtccgtcaggtaaggttcagagagacgagcgcaagcgaaccgctgatgacgtgtcgaaaagtgcaagatgatgtcgaaaccgggaggaattcgttagcccgggacgagtctggggggcgacctgatttctgcccagacggcatccggcatgaaggcggcgtgactcttcaccaggctttcgtgtggaacgcgggaacctgtcgctccgatgttaagggagaaacccaagcaggcggtccctgcgagggtgagattaccgaagcggggcacaggggcggagcagcccgtagtagtgctgaaggtcctgtaatggggctggagcgaaggggttgcgttgttcagctctggagcgtggacaaccggcgacgggaggatcctcgtggatagagcaaagccgtatacgatcccgaaaagggaggtgtgggaagcatataagagggttagagccaaccaaggagcggctggtatagatggccagacgattgcggacttcgaggctgatcttaggaacaacctgtacaagctctggaatcggctggcgtcgggcagctactttccgccgccggtgcggcgggtcgacatacccaagagcgatgggaagacgcgcccgctgggcatacctacggtcgccgatcgcgttgcgcagatggtggtcaaacgccatctggaaccggtggtggagcctgaattccatccggattcctatgggtatcggcccggcaaatcggcacttgatgcgatcagcgtggcacggcagcgatgctggcgttacaactgggttcttgatctcgatatcaaggctttctttgatagcattgaaccggacctgctgatgcgggctgtgcgcaagcacaccgattgcccgtgggtgcttttatacattgaacgatggctgaaagcgccagtgcagatgccggacggaaatctcgtcgccagagaacgaggaacgccacagggcggggtgatcagccccctcttggcaagcctgtttcttcactacgcgtttgacatgtggatgtgtcggaatttcccggacattccgtttgaacggtatgctgacgacgcgatctgtcattgtcggagcgaggatcaggcgatggcgcttcagaacgctctggacgcgcgttttactgactgtgggctgacgcttcatcctgacaagactaaaatcgtctactgcagggatgaaagtcggcgaggtactcatccggtctacaagttcgactttctcggctacactttccggccgagacttgtgagtaagaaggccgggggcatgggtgtttcattcggtcctgcggccagtccgacggcgctcaaggcgatacggggcacgatccgtagttggtccttgcaccttcgcagtgacaaagctctcgatgatctggcacggatgttcaactcgtacatccgcggctggatcaattactatggtcggttctgtccttcagctctccaacctacgctttggagtgtcgagcgatacttggctcgatgggcatcgggaaagtacaagtccttgcgtaggcataaacggcgatcccggcattggcttttgcgcatcgcgcagcgccaacctcggctgttcgcccattggcctctgcttcatggatacggtcgaacaatgggagccggatgatgcgagagtatcacgtccggttctaagagagcgtgggggtggaactcccccgcgccactcgcctcctgatttc

SEQ ID NO: 181 - DNA, 2512 bp, Alistipes shahii
  source 1..2512: mol_type = genomic DNA
aagtatttgtgtgcgcccgtacaagctgcttagtaaatatcccactggcaagggattaggtaagtgttctttgaaaaacctatcatcacccgacttatccgaaagggaaaccggacggggagtgtagcatgtcgggaacgtcgtaagtcagtcagttaccagactgccactgaacggcaagatgaaacgatggatatgaggatgaaaaccgaattgtttgaacgacagtccgagctgctctacttgaaggctatgacgtaaggtaactgaaatcgtcatactgcggtactgctccggtgtataggtacggacgcaagcaagaacttgccgcagagcaaccatgataaatggtaaaggacggaaatcgcatccgacaatccatcatgccaacagttactaagtaagtaaacggggattacctaaaccggaacgccgtaaggctatgaggtgcagacctctgaatatccggcacggtaacggagcctccgtagtagtccgggcagggtaacgccctgcacatggcgaagggaggcagctaattttctttaatacaatttaatggataatgtgagagacattatgagaagtccggaaagagtattaaacagtctgaacgaacacagtaaggattcgagctacaagttcgaacgcctttaccggattctgttcaacgaagagttgttctatgttgcctaccaaaagatcgcatcgaacggaggcagtacgaccaaaggctccgacggtcgcagtatcgacgagatgagccttgcccggatcgaaacgctgatagcctcgttgaaagacgaaagttatcagcctcacccgtcgcggagggtgcatatcccgaagaaaaacgggaaaacgcgccctctgggaataccggcttttgaagacaagctggtgcaggaggtagtccgcatgattctggaagccatttacgagggacattttgagaccacctcgcacggtttccgacctaaacggagctgtcacactgcattacttcatattcaaaagacgttcagcggggcgaaatggtttatcgagggcgacatcaaagggttcttcgacaacattgaccatgatgtacttgtcggaatcctgcgggagcgcatttcagatgaccgttttatccgtctgatacggaaattcctgaaagcgggatacgtcgaggattggacgtttcacaacacttacagcggaatgccgcagggaggtatcgtcagcccgattctggcgaatatatacctcgataaattagacaagtacgtgaaagagtatatccgacatttcgacatgggaaccaaacgcaggccgggcaaagagagcaacgatttggccaatgaacgaaaacggactgtgcggaaactgaaaaagataaaagacgggactgagaaggcggctttggtcgcaagactcaaagccatcgaacaggaacgtgcagcatttccaagcggagatgaaatggacggaagttaccgcaggctcaaatacatccgttacgccgacgattttattctgggtgtaatcggtagcaaagaggatgcactgcggataaaggaggatattaaatcattcctatccgaaagcctcgccctcgaactgtccgaagaaaagacgctgataacccatacgggtaaatcggcgaaatttctcggatatgagattacggtaacacggaacaatcatcaacgacgggatgtgcaaggacgtctgcgacgcacctacggcaagcgtgtccggctgaatgtcagcatggcaacgctgcgggacaaacttctggaatacggagctatggaaatcaagctccgcaacgggaaagagatttggaaacccaaatgccgttcaggattgatattcaacgacgatcttgaaatcctcgaccgatacaatcgggaaacagtgggattttgcaactattacctgatcgccaacaactgcgtcgtactgcacaacttcagatatatcatggagtacagcatgtataaaacatttgcgggcaaatataggagcacggtacgaaaaatcaacaaaaagtaccgtctcaacaaactgttcaccgtaaagtacgagcagaaaggggtaatcaagtcccgaaccttttacaagacaagtttcaaacgccggacaacggcgttcaacggaagctgcgacatcgaaccgtactctatcgcagacgcgagccgaaccaatttgacagacaggctcaaagcggaaaaatgcgaattgtgtggggcaacgggcaagctgattatgcaccatgtccgcaacctcaaagacctgaaagagaaagagagttggaaacggctcatgtcagcccgaaaacgcaagaccattgcgttgtgtccgagttgccacaggctgcggcatctgggaaaagtttagactgaaaaaaaaaattagtggagagccggatacgccgagaggtgtaagtccggttcgggggcgagttcttggaaacctgccgtagaaatacggtaaggcgccgggtgcttagcctacataaaacctg

SEQ ID NO: 182 - DNA, 2007 bp, Wolbachia sp.
  source 1..2007: mol_type = genomic DNA
tcagtaccaagtgcgtccagagaaggcgagtattgttcagtaggaaatagacctaccaggataaaagtcggaatcctgtagcttgggtagcagcataaagggaaaccaaaatgttgaagcctaccgacaacattatcgtaggataatggcgaattaccaggccgtagcgaaagcgaatgcacagattgcctcgaaaggaatattctgaaggtcgagccatcttggtactggcgaagacagaatagaccagacgaaaaacgaatgaaacgattggtctacttcagcggggtgttagtaacagcatggcaatgaggacatacaaagcaactggagaagccctcctcatcccaaggagaaacaccttggagaggtaggttctataactggaaaacaggaagtgaactgaagatgagagggtgacggatgggtgcgtagtagtgaagaagtgaggtaacgctcatggagcaaaggcaccctactagcagggatcttttaacaaaaggaaggtaaaggtaaaaatgacaaaaatgccgataaaattacaagacctgagaagaaagatatacaccaaagcgaaggcagaaccggaatggcgattctggggaatccatgttcatgtctgtaaaatggagacactggaggaagcttacaaactaacgaagaggagtaatggggcaccagggattgatggggtgacattcgaatcgatagagacagagggctcaagaaagtatcttcaacgaataagacacgaattaataaccaagacctatagcccaaataggaaccggagaaaggaaataccaaaatctggagaaaaattcagaacgttgaacataccctgcatccgggatagaatagtgcaaacagcactgaagctaatacttgaaccgatattcgaatcagacttccaaaaaggatcatacgggtatagacctaagcgtaatgcgcatgaagcagtacaaaaagtaacggaagcagcaatcaaaggcaatacaaaagtaattgacgtagatctaaaatcctactttgacagtgtgcgacatcatattctgatggaaaagattgccaaaagaataaatgacaaagaaatcatgcgcatgattaagctaatcctcaaaataggaggaaaacgaggaatggcacaaggatcaccactttcaccactactaagcaatatatacctaaatgaggtagataaaatgttggagaaagcaaaagaggtgaccaaagaaggaaagtaccaacgtatggaatatgccagatgggcagatgatttggtgatactgatcagagaatacccgaaacgggaatggctagaaagagcagtatacagaaggttggaagaagaattagcaaaactagaagtgagagtaaatgaagaaaagacaaaagtcattaacctaaagaaaggagaaacatttagcttcctagggtttgattttcaagagaatatcacaaaacaaggaaagtggaatgtcaggaaaacaccgcagatgaaagcacgaacaaacctgctacgcaagttgaaggaaatatttcggcgccataaatcgcaaccgataaaaagagtgataaatgacatcaatccgatattgagagggtgggtaaactactttaggataggaaactctagtcgaaagttcagttacgtaaaacactgggtggaaaagaaggtaagacgcaatttaatgcgagccaggaaagcaaagaaagggtttggttggaagaaatggagtagcgaatggatatacaaaacgctgggtctatattcagactacagaataaggtattacagaccgaaagcatcaccaacacaatatgtgataaactttggtaaagaacttgctaggaaagcgtagtgcgggaaaaccgcatgctgcgtttgacgaggcgggagctggaaacgttaacatgggaatttggattgaggcccaggatgaaaataatggatcagccaccaaaccctaaagaggcgcgccagttctcgaccctacagatgcgctt

SEQ ID NO: 183 - DNA, 2422 bp, Xylella fastidiosa
  source 1..2422: mol_type = genomic DNA
tggcaagctcgtgcgacgtgaagtcgcttgtttcattgcttcaccgcttcggcgtgatgaagcctgatgttccgtcatcagtagcctatcggtgcatgcgtggttggcaacgactgcgtgtgaagccaccgagacaatgtaggtcggcgaaagccaggtggagaccgcgaggtcgaaaccgcatctgcgagcgccaacgcggatggatcgctagactggtcggtatggttaacgtatgtgaagtgctgataaacaccgtcaagatgaaagagccaaagacgctgacaggctctacccaaaatggggcgtagctggacattcctctgagctttgggaatgcacggacacgaaagctaccggtggagaggcgcaacctaacctgacctgtgaatcaagtggaacacggtaagcccgtatctctgcccctcggggcagacagaccgtaaggaaagttcatggagatgcgggtagaggaatgtggaaaaagcgaatgccgccctgtaatggggcggataggggctgagccgcgaggcaacatcgcttcacgtgaaaacgggcagacttccacttggtctttctttacgagattgtttgaagaacctttttaggagagaaagcaaatgactacgcaagctgcttgcgcgggtgcgctctccggcgactcgggcggatggcactgcatcgactgggccaactgccaccgggaagtcaggaggctgcaaacgcgcatcgtaaaggcgacacaggaaggccgctggggcaaggtgaaagccttgcaatggctgctgacccactcgttcagcggcaaggctcttgccgtgcggcgggtgactgaaaaccagggcaagaagactccgggtgtggacaaggtggtatggggcaccccggaaaagaaggaaaacgcagtagatgagttgcggcgccatggccataaagccagaccgctgaggcgagtctatatcccgaagtccaatgggaagctacggccattgggtatcccaaccatgagagatcgcgcgatgcaggcattgcatctgctaagtctgctcccggtgtcggaaacccaagctgatggatgttcctacggcttccggcccgagcgttcagtggcagatgcagtcgataggtgcttcagggcgcttaatggacaacattccgcccaatggatactggaggccgatatcaagggatgcttcgatcacatcagccacgactggcttctgggcaacgtgcccatggacaagcggatgctggaaatgtggttgaagtgcggcttcatggaagcaggtgcatggtcggaaactgaggcagggacaccacaaggcgggatcatctcgccgacactggcaaacatggcgctggacgggctggaacaactattggaacagtccttccgctcaagaatgcgagatcggcagatcaccaacccgaaggtcaaccttgtccgttatgccgatgactttgtgatcaccggagactcagagaagcttttgatcaaggaggtcaagccactggtggagcgattcctggcggaccgtggactcaccttgtctgcggagaaaaccagggtgacgcacatcgacgaaggcttcgacttcctcggccagaatgtgcgcaagtacaaagggaagttgctgatcaagccatcagcagcgagcctcaagaattgcgcagacaaaatccgggccatcgtacggtcgaacaaggcagccaatcaggcatcgctaattcacctgctcaaccctgtgatccaggggtgggcaaacttccatcggcatattgttgccaagaagaccttcaagtctttggatgatcatgtctggagggccctgtggaaatgggctctacggcgtcatccggcaaaaggcaaaaagtggattcggcatcggtatttccatgcgtagcgtggtcgtcaatgggtatttgcagcccgatacgtggactcttcgagcaaagaggctcgatgggcaaccctccgtaaggcagtagacaccaagatcaggcgaaatacgatgatccgcatgggtgcgaatccattcgacccggagtgggaaacctactttgaacagcgcctcggcgccaagatgaaggacaacctacaaggtcgaaaacgtctattgcacttgtggttggaacaggacgggaagtgtcctgtatgcgacgaaccgctgacgaaagaaagcggctggcatgtccaccatatcgtccgtcgcgtcgatggtggttccaatcaaagcgggaacctggtgatggttcacctgaactgtcattaccagatccacaaccttggtttggatgttgtgaaaccggctccagcaatggggctttgaaaggcttgagccgtatgaggggaaaccttcacgtacggttcttaggggaggatgtggtggcgacgccatgtccttacccgaccacataggca

SEQ ID NO: 184 - DNA, 1884 bp, Zunongwangia profunda
  source 1..1884: mol_type = genomic DNA
ggatgaaagtgtgtgccttgaagcggcctaaagtaatgccgcaggctgttagattgatggtggtagacccaccgtaggggttgacagacagaccctacaaaccaccttgtggtgctttatttcgaaagaatgaggtgctgagcgacaaggaaaaggcgagagaatactcgtcaggtgaatttcagtgagctgaacgaaagtgaatcactggttaagttaaagtaccgatatcgccagattctgtcgaaatcggagaattgtgtttcttctccgagaataaaacttagtggcagtaacctgaagttgactaagtgacaggcggtacagaggtggcaggacactgaaataggcgatctaacggaacaagggaatttgtacagggatggtaagggaaatgcacaagcggaaaccccgtgaggcagaaaaccaatgaactgtacagaggcggactaagccgtagtactgcagaagtctctgtaatggagatggagggaagggcttagccagattagttttatgtaagacacaactttgaaagaaggatgattgattattatgaaacaaaacaacatccaataaccaagaaaatggtattggatgcgtacaagaaagtaaagtccaaaaagggcagtgccggagtcgatggacagagtttacagaattttagggagaacctctctggaaatttgtataaaatctggaaccgtatgacctcaggcagttattttccgcctgttgtaaaggaagttcgtataaccaagaaaacaggaggctttcgcagtctcggaattcctacggtatcggatcgcattgcgcaacaggtgattaaaagttacttagaacccaaggtggaaagcagttttcatcagaatagttatggatacagacccaggaaaagtgcgcatcaggcactggagaaaacagtgtccagatgtggttattacagttgggtggtagaccttgatattcgtggattctttgataacattgaccataccctactgatgaaggcagtagaaaggtatacaaaagagaaatgggtgttgatgtatattggaaggtggttgaaaacaggagtatccagagaaggtgaaatcaccgatagaataaaaggtactcctcaaggcggcgtcattagtccgctacttgccaatatatttcttcactttgcattcgataaatggatgcaaatccatcactccaatatgcctttcgaacgatattgcgacgatgccatcatacattgcacatcagagaaacaagcatatttcattagagaggctgtttctaaacggatgaaagcttgtaagttggaactcaatagtgagaaaactcacattgtgtattgcaagaatcacgtgcacagcgaaagtcacaagaatacaagttttgattttctcggttacacgttccgccccctcagacgaccaaccaaaaatggttggaaactgacttactttccagtaatgagccacgcctcaaagaaagaagcaaggcgaagacttaagaaggttgtcaatagaaagtttcggggatccatccaagaactggcacagataatcaaccctatgataaggggctggtatcaatatttttgcaagtattcgaaatggacaacccgtggactatggtattggttaaacaggaaaatagttagatggattaggaggtacagaaagcgcagtagagtgcaagctcgaaaatggctaaaaaatgtgtacaagactaacccacaattgtttgagcattggaaacctgtcttagatataaaaccttactaattgtaccgttaatctggaagcgccgtatgatgggagactatcacgtacggtgctgtgagagacttggggtgaaattccccttgtctactcgacccgataaagg

SEQ ID NO: 185 - DNA, 1906 bp, Zunongwangia profunda
  source 1..1906: mol_type = genomic DNA
acttgcgatagtgtgccgagcaaacaatgaccggcaataaatagtcattgtgaactataattaagatggtagaatcgacctaccggtgtcgtcttataggagaaggcatcaaaccaaccaaagatgctttggtaaagagccaaggtattgagcgtttaggaaacggccagtcaagaattggtcaggtatgattaagagagatgaacaaaagtgaaccccttctgaggtgtcgagaagttgctacatcctgtcaaaagccacgaagtatcgcacggggttaaagagtatagcagttacctatttattggctatacggcaggcgtcattcagggggcatgactcttattcaggcttagttatggaactcgggaacctgactatagatgctaagggaaatacacaataggctcaacctagaggtagaataccaatgctgttttcagggacggactaacccgtagtagcaatgaagttcctgtaatgggaatagagcaaaggggttaggttatacagttacgttacatttaacaactcagttttgaggatgactttatgcaatgaaacaaaatcaatacctataagcaaagctatgatatggcaagcttataaaagagtgcgcgccaacaaaggaagtgcagggatcgatacagtaagcattgagcaattcgatgagagtttatcaaagaacctgtacaaactttggaatcgtatggcatcgggaagttatttcccccctgcggtcaaagaagtggaaatcccgaaaaaggatggtaaagtccgtaaattagggattcctacgataagtgaccgtatcgggcaaatggtggtaaaaatgtacctggagccccgtttagaaaatgtattcaatccaaactcctacggatatagaccaaacaaaagtgcccatcaggcacttgaacaggtcaggaaaaactgttggaaaatggattgggttattgatctggacattaaaggtttttttgataacattgaccatcataaaatgatgcttgctatagaaaaacacgttcctgaaagatgggttaggttatatatagcacgttggctcgcaagtccagtaatgacaaagtcgggaaatttagtctctaaccagggaagaggaactccccagggaggggttataagcccattactggccaaccttttccttcactatggtttagataaatggctggaacagaacgacaatacggtgaaattcacacggtatgccgatgatgtgattgtgaactgtaaaagtcaaaagcacgcagagcaaacacttgaagctatcaaaagcaggatgcatcaaattggtctggagttacatccagagaaaaccaaaattgtatactgtagagattaccgaaggcaagaaaagtattccaatgtgaagtttgattttctaggttattcttatcaacctagaactacaaaatcgaagaaatcaaacggactctatcttggctttgattgcggcataagtataagttcgaggaaacgaattgcagataaacttgaagaattgaaagtagagcgaatgacttctgatcgtatagtaggaattgctgccattctcaatccaatgattaggggatgggtgaactactatggtaaatttagaaggtcaatgctacacaaagtcttcaagttattgaataatcgaatcgtgaaatgggcaagaaaaaggtataaacgttacaaaaccagtataaaacgtgcctatcaatggtttgaaaggattaaggaacaatatccaaaactattttatcattggcaggtgggatttgtatggtaatttgtaatttagattgtataacaagagccgtatgatgggagactatcacgtacggttctgtgagaggcttggggtgaaattccccttgtctactcgacaccgtaaccg

SEQ ID NO: 186 - DNA, 1950 bp, Vibrio vulnificus
  source 1..1950: mol_type = genomic DNA
tcaagtaaaagtgtgccgtgggcacaggatcctgttatctaaggaaggtaaacaggagaatgagcataagctcatgaagagatggaggtaggccctccagagtctgctgtcggaagtcggctctaaaccactttcaagtggctgttattaagagtaatggtcagaagcgttgaaggaaagtcagaatagaattctggcaaggtagagtgcaaggagatgagtaatcccgaacccatgtggacgcgtcgttaattagaaccagcatcaaaactgaggtcgttttattatctcaggacgagtacaccggtaaattccgaatgctgggtgtgcggtgctcggcgtaaagtgggcatgactttgcactaggctctttgtgggaaccacgggaaccagtcatcacgatgtaaaggaagaaatataaatgactaaattcatgagtatgagagtaccgatgcgtgatactggggcggagcaattcgtagtagtgatgaagggcttgtaatgagcgtggagcgaagggattgcgtcaagtcggttggaacgatgactcaactgccgacaggcaggaggaggtcgcacggacaaccaaaaccttttgcaatatccaaatgggatgtaatgactgcgttcgagaaggtaaaagccaacaaaggcggagccggagtggatggagtaacgatagaggactttgaaaaagaccttaaaaacaacctttacaagatatggaacagaatgtcatcggggtcctactttcccacaccggtcgcagcggtaagtataccgaaaaagtctggtggagagagggtattgggtatcccaacggtcagcgaccgagtggcgcaaactgtggtaagagacaagcttgaaatcatgctcgagcatcacttcctagatgactcttacggctatcgagtgggtaaatctgctcatgatgcgatagaagtcactagaagacgatgttggcagtatgattgggtactagagtttgatatcaaaggtctctttgacaatattcgtcatgacttgttgatgaaagcggtaaagaaacatgttcagctcgctgaagagagtcagagccgggattatcaatggataacactgtacatcgaaaggtggttagttgctccgttacagaaagcagatgggacgcaaacagaaagagagttaggaacgccacaaggtggcgtggtaagcccagtgcttgccaacctatttcttcactatgtttttgataaatggctggagaagaattatccagacaacccatggtgtcgatacgcagatgatggacttgtccatgcaaggacaaagccgaaagcggagaagttgagggatgagctagcgaagcgcttcaaggagtgcggactggagatgcacccaattaagacgaagattgtttactgcaaagatgatattagacgagggtcaggtaagcatatagagcataaacaatttgattttctagggtataccttcagggccagaacaaataagtgtaaacgtacaggtcaactctataatcgattcctgcctgcggtcagtatggcagcaaagaaagtcatgcgaaggcaaatcagagagctaagagttcgccaggaaacgcagtacagcttagagcaattaagcagatggttaagtccaatgctgaatggttggataaactactacggaaagttcaggcgaagcgaattagactcggtattcagacacttcaacaagactttagtacgttgggcgagaaggaaattcaaatcgctaaaatgtcacaaaagtcgaaccgtagcgttctttgataagctatcggctcaatgccctagattgtttcctcactggaaatttggttcagcaagaagttttacttgatgggagccgtatgagctgagaggttcacgtacggttctgcgagaggctgctggggtggttccggtggcctactcacctcagttggtt

SEQ ID NO: 187 - DNA, 2036 bp, unidentified
  source 1..2036: mol_type = genomic DNA; note = unknown marine bacterium
tgagattatgtgtgcacagaggattgagcgagagtaaagcacatttaccccacataaattgctgaatcgaactatagataagatggtggactgtaagccatattttaaagtttatttaaatagaagattaaytgtagaggtcgagcgaacagactggctgagttagtcaataaatagctttaaattacaggtaaaaaaaaacaggattgaaccaccagcagggactgtcaagagtatctgctaaaccgccttacggtgcttttatttaacaatagaggtattgagcggctaaggaaaaggggcgatgaaagaatcgtttcaggtgtttgtacaagagctgaacgaaagtaaactgaccgatgagatgtcgaaactcatttaggcagtgttggaaaccgagggaatataatctgcttcgggacaagcacaagggtaacttgaatataccacttgagccaacactcggcgtataggcagcaggcttgtatatgggcttgtttatggaactgtggaaaccatcatatgttgtaaattgaaacaacacaagcggtaaaaccrcgaggttaaaagtagagatacatatgatggtggcggattaagtcgtagtagtgaagaagttcttgtaatgagggtggagcgaaggacttaagcaaacaggagttaacaattgcaacaacttaaaattaaggatgattgaggcgttaagaccaaaaacacaaccagtaagtaaagagcaggtatggtctgcgtggaaacgagtaaagcgaggaggaaaaggcatgggagttgaccatgttagtatggaagccatcgcatcgaatccacggaaatacctgtatccattgtggaatagactatcaagtgggagttattttcctccacctgttaagttagtgcctattcccaaaggagatggcaaagaaaggatgttgggcataccaaccataatcgacagagtagcacaagaagtcatcaaggcggaattggaagtcatagtcgaaccgaggtttcaccccagttcatttggctatagaccgcacaaaagtgctcatgaagccttggaacaatgtgcaaagaatagttgggaaaggtggtatgtggtagatttggacattaaaggattctttgacaacatagaccatgaaaagatgatggggatattacgaaagcataccaacaagaaacatatcctactttactgcgataggtggctaaaaacccctatgcaagacagggttggaggggtacaggcaagaatgaaaggaacgccacaaggaggtgtaataagccctttgttggcaaatctttatttgcacgaggcttttgaccaatggatatcgacaacacaacctcgtatagtatttgaaagatatgccgatgatattgttattcatacaagaagtatggagcaatcgcattttatattagacaaattaaaagcacggttgaaaagttattctttagagctacatcccgataagacaaagatagtatattgctatcgcactgcgcggtttcacaaagaaggtaaagagatacccgtatcttttgatttccttggttttacttttaaaccacggctttgtttaaaatcaaacggtgagaaattttggggattccgtcctgcaattagtaaaaagagtgagaaacggatattaggagagttacgaaagttaaaaatacatagatggatatatatgaatattcatcaagtatcacaacgcttaagagcaaaggtacgaggttggctaaattattatggtagatttcgtaaatcagagcttagttggattttcaggcatctaaacatccgaatttcaaaatgggcacggaataaatataagttaaagacttattccaaatcttatgggtggcttaaacgtatggtaaagtggtatccaaatacatctttacattgggagcatggctttacaggttaggtttgtttggaagagccgtatgaggggagactttcacgtacggttctgtgagaggtttagggtgaaatcccctttacctacttgactcgcttgtgt

SEQ ID NO: 188 - DNA, 2735 bp, Crocosphaera watsonii
  source 1..2735: mol_type = genomic DNA
ttatacttctgtgcgattcgttgagctaaaaagctgacatccaagtacatcacaaggatacagcgtgtagctcattcaagagccttctaaaggcaattcaatggttaaacgaccaatctaccccaaagatgaaaaacaagagccttaaagctcaattttcaaggggcagaacacaaaggtcagaagggtaaaaacaatacccgaacggtaaaaaccaaagaaaagtttacaaacttaagtagtaattaccgaattaactaagtagggataaccgaacagtaaacgaacctagaaacttgaataactgttggtttgttggctaaaggagccatactcggaatcaagggagaaatcccgagagtaacgacataaccctccgttttggacttagtaaacccaggacggaagcagggataaaaggggatacccactgtgaaacagcacaagaaatcctcaagtcaatcggtcacggttaaacattacgttaaatcatcgacttgaaccctcaaaaaaccgaagtagccaaatctgtttgataggctgtgttcaaaagaacaaggtgaaaagtggagtttttgaagtaacatccgcacagattcctaaaagtaacctgggggagagatgaaacctaacaccgatatacccaaaccaacggaacgaattaagtccatgattgctcttaccactggtcggtaggtaagtaggaacaaacttaaccgcaagggtcatggatgtgagaagcaataagaagctaatgcctacttgtaataagtatggatatgctgacgtattgctggtctattagtaagaaatagtctttattagaggtagttaatgtcgaatgcgagtataaccaagactacgccagaatggaacacaatcaactgggcaaaagtccagaggaaagtgtttaagcttcaaaagagaatatttcaagctgttaaatcaggtaataaagttaaagctaaaaagcttcagaagttactgttaaagtcacactacgcaaagctcttagccatacgcaaagtgacccaagacaaccaaggcaagaaaactgccggagttgatgggaaaaaggcactacaccctaaccaaagaataaagttagtcaaagaattaactttaaaaggttacaaagcaaaggcactcagacgggtatggattcccaaccacagaagagatgaaaaacgtggattggggatacccaccatgaaagatagagtaatgcaagccttggttaaatcagccttagaaccatattgggaagcccaatttgagggaacatcctatggttttcgtccaggcagaagcgcacacgatgctatcaccagaatatacacagcaattaataagaaagctaggtatgtactagatgccgatatcgccaagtgctttgacaagattaaccatgattacctactgtctaaagtagattgtccacacaacatcaaaagaatcattaaacaatggttagaatgcggcgttatggacaaaggcatcttagaggaaaccgattcgggaacacctcaaggtggtgtaataagcccactattggctaacattgcactacacggaatgataaaagatacagaaaaccacttcccccgtacaaaacgtaaggaagatggaagtctacacagggagtataaacccaaaatcatccgttatgccgatgatttcgtagttctccacgaggattacgacgttattctacaatgtaaaactttaattgcacaatggctagaaaaggtcggattggaactcaaaccagaaaaaacctccatcagacataccctaaaaagtattgaacatgacgggaaaaccattgaccccggtttcgatttccttggattcaacataaggtcataccccgttggaaaacaccactccggtaaaacaggaggaaattcaaaatgggggatagaatccaaagcaataggtttcaaaaccttaatcaaaccaagcaagaaaaagatactggcgcatcatgaggcaatcaaagaggttgtaaaagccaacaagaaagccccacaagaagtgttaatcgcaagattaaaccccataatccgaggatggtgtaattattaccgaactgtaacaagtagagaaacattctcatccgaagattcaatattatggaatacacttagagcctggacagtaaacaggaaaaagaaagagacaccactgtatgatgccctcaaaaaatatttctcatacggaaaacaagggaaatggacattccaaacaagggaatatgtcctctatcaccacaccgaaacggaaattaagagacatacactggttaagcctgaatcatcaccctacgacgggaactggacttactggagcaaaagaagaggtacatatactgggacaccagccagggttgcgaaactcttaaagaaacagaaaggtatatgccctcaatgcaagcaacacttcacacctgaagacttgatagaagttgaccacatcatcccgaaatcgaaaggtggaaaggatacttacaacaacctacaagctctacatcgtcactgtcacgatgccaaaagcaaaaatgactacctctatgattggcacttataatggctatgaatggaaagacgatgtattaacagtaccctagacaagggactaattgccgagagccgtgtgaggtgaaagtctcacgcacggttcggaatgggaggagaggaaggtgacttccttttcgacccctaatcatctatccc

SEQ ID NO: 189 - DNA, 2609 bp, Clostridium sp.
  source 1..2609: mol_type = genomic DNA; note = cf
gaaatttaccgcgcgtccagaagcgccgcttgtcatccgcaggcggtgtgcggacattttgggaatccggctttggcaagccggtaaagaaaagtatcaaagacgctgaaaaagtgtcgctttagcgtcagctggtttaccccggagggaaaccacaagggggacaatagcgtaccggaaacgtcgcaagttgggaaaaccatctacccacgactgagcggcaagacgcaacattgtgaacaaggaataaggctaaactgctttaagggcagtccgaggcgggatactcgacccggcggcgtaggatggttgtattcgccgtatgtcatagcggaatgtgacaggtatacacagaccgcaggatacttatgacagccagccgcagtaagcggaaaaggacgaaaacccactccgacatcacaatacgcttttcccaaagtgtctaactggagattgcctaaaccggaacgccggaaccatccggctatgcgtaatgccgcaaggtgaaaaatttcaaggggtaaaaacctaagaaagatgacgctgaatatccggcatggcaacggagccttcgtagtagtccgaggacgggaaagccgtctacatggcgaaggaaggcaggatgtcaaccaactcataaaaaggaaaggtgcgtgaggcattatgagaagtcctgagaatgtgttggaaagcctaaaatccaaggcgtgtaaccagagttacaagtacgagcggttataccgcaatctgtacaatccacaattctatctgctggcataccagcggatacaggcaaaaccaggcaatatgactgccggaacagacggcaagacgattgacggcatgggaatggcgagaatcaacgccctcattgaaaaaatgcggaattccagctaccagcccaaccccgcaaggagaacatacattccgaaatccaacggaaaaatgcgccccttgggaataccgtcattcgatgacaaactgatacaggaggtggtgcggctcattctggagagtatctatgagccaaccttcagcgaccactcccacggcttccgcatgaacaaaagctgccatacggcactcaagtatgtgcagaaatatttcacggggacaaaatggtttgttgagggcgacataaagggctgttttgacaatgtagaccatcacgtgctgattgccattctgcgaaagcggattgcggacgagcagttcattggtttgctctggaagttcctgaaagccggatatatggaggactggaactaccacaatacttactccggtactccgcaaggctctattatcagccccatcctcgcaaacatttatctgaacgaattggatcacttcatggcggaatacgcagagaagttcaactgcggagatcgccgtagaatcaatccggcgttcaagaagaaattggatgtctgccgggggaaagaagaacggctcaaaagaaacatctccaaaatgagcgaggaggaaaaagagggcttgctcgccgaaatcagcgaactgcggcgtagcttgcggtcaatgccgtacagcgaccagatggacgaaggctataaaagagttttctatatccggtacgccgatgattttctgattggagtaattggcagaaaagcggacgcagaacaggtcaaacaggatgtggggcactttatccgggaaaatctccatctggaaatgtccgaggaaaaaactctgattacccacgggcatgactttgccaagttcctgggatatgaggtcacaatcgccaaaggtgagtgtaacaaaaagaccagaaccggagccaccagacgagtaaacaacggaaaggtcctgctctatgtcccccacgacaagtggatcaagcggctactctcctatcatgccctcaaaatcagatacgacaaacagaatggaaacagagaggtttgggaacctgtccggcgcacccgcctgttgcacctggacgatttggaaatcctgaaccagtacaacgcagaaatccgtggattgtacaactactatcggctcgcaaacaatgtgtctgtcctcaacaacttctactatgtaatgagatacagtatgctcaagaccttttccggaaaataccggacacgaattagcagaatcattcagaagtaccgtcaagggaaagattttgttgtggagtatccgaagaaaaacggccaggttggaaaggtgctgttctacaacaacgggtttcgccgtgataccaaagtggaaagcggcaatcccgatgtgattgcaagagtgtttgagaactacggacgcaatagcctgataaagcggctgaaagcgagtcgatgtgagtggtgcggtgcagagaatgtaccgattgaaatacaccatgtacgaaaactcaaagacttaagcggcaagaaacaatgggaaattgccatgattgggcgcaagcgcaagacaatggcactctgcgttgactgtcacgataagttacacgctgggaaattagactgacatctggagagccggatacatcgagaggtgtaagtccggttcggagggaagttctcggaaacctgccatagtgatatggcaaggcgctgggttcttaccctacttgtcacgtc

SEQ ID NO: 190 - DNA, 1874 bp, Clostridium saccharolyticum
  source 1..1874: mol_type = genomic DNA
gcttttttccgtgcgcccagcatgggcgcactcttggaggtgcaagtcctcccgtgagttgatcacagcgaacgaagcgaagcgcaactgcatgagggtgaccgagtgtgggaaggaagcgtggagcgcaactgcgagccgatggacaagaaccggatagaaggcggtgccgagcagggcgagcgggcagaaaaccgcgaagctctcgtgatcaaggcgaggtgtcgtaaatccggcggttgtgcagtgaaggagtgcgttcttacctggggagatctcgccttgtgcctgaaagggcgacgtcgagaggcggagcgagaagtcagccgaggtcgtagtagtcaggggctgggccggtgaggctgaaaccgctggcgaaggaccgaacgagagggagtgtagaccgacatgtcgatgcggcaggccatgcgtcagatgcccgcgcaagcggggcgggcgggagtagcgcgcggtgaagccgcgcgtgtacccgtcagcgacgaagcctgcggcccgcggcgtgagccggaaaacacagggtcggcgctgctgcaggcagcgctgacgagagagaacctgcaacgggcgctcaaacgggtacgtgccaataaaggcgcagcgggcgtcgacggtctggacatcgaccaaacggcacgccatctcgcgacggcctggcccggtatccgtgagcacctgttgcgggggacgtaccggccgagtccggtacgacgggtagcgatccctaagccggacggcggcgagcgcgagcttggcatcccgacggtgacggatcggctgatccagcaggcgctgctacaggtactgcaaccgatacttgatccgagcttcagcgagcacagccacggcttccggccggggcggcgtgcgcaggacgcggtgcttgccgcgcaggcgtacgtgcagtccggccggcgcgtggtggtggatgtggacctggagaagttcttcgaccgggtcaaccacgacatcctgatcgaccgcctacagaaacgcatcgatgacgccggggtgatccggctggtgcgtgcgtatctgaacagcgggatcatgagggatggcgtggtgcaggagcgcgggcaggggacgccgcaaggcggaccgctgtcgccgctgctggccaatgtcttgctcgatgaggtggacaaggccttggagcggcgaggccactgcttcgtgcgctatgccgacgactgcaacgtgtatgtgcgcagtcgccgggcgggagcgcgggtgatggctttgctgcggcgtctgtacggcaagttgtgcctgaaggtcaatgaagccaagagcgcggtagccagtgtgttcggccgcaagttcctgggctacagcctgtgggtggcccggggaggaacggtcaagcgcaaggtggcggccaagccgctggcgacgttcaagcagcgggtgcggcaactgacccggcgcctgggcgggcgcagcatgcaggacgtggtgcaaaggctgcgaccttatctgctgggttggaaagcgtacttcggactggcgcaaacaccgaggatctggcgcgagctggacgaggggttgcgtcatcggctgcgtgcgatccagctcaagcagtggaaacgagggccgaccatgttccgggagttgcgcgcgttgggcgcaagccccgccgtggcgcagcgggtggcggccaatagccgctgctggtggcgcaacagcgacaaactgctcaacagcgtgttgacggtggcatggttcgaccgcctgggtctaccccgtctgtcctgacctcaacctctcgaaccgcccggtgcggacccgcatgccgggtggtgtggcaggggcgcagtctacgatgactgtcccctatgccgattccgcacagc

SEQ ID NO: 191 - DNA, 1935 bp, Burkholderia cenocepacia
  source 1..1935: mol_type = genomic DNA
tacgtacagtgtgcgccgggagaccggtagagatcaaggggtgaaagtccccgacattgcaagaactagcgattcacgatttccccgagtcatgcgtggccagccgcgaggcgggttgcgaagcgttgacaggggcgctgataggccagccattgagccacgaaatggatacattcccgagtgctgacgtactactgtatgcggaaggcaacatcgcgcggcgcgctatcgcgagcgctgcgcggactcggcgtggtctgagaccctggcatgtcagtacgatctctacgcgggaaccgggagatctcccatctggccgccgacggaaatatggtcgagcggtccgcatcgggaaggcgaggagctgaagccgatgacgaacggatgggagaagtcggactcgcccatagtagcgaagaagctggcgaacaaacccgggcaaccggatgcggagtcagtggagcgaaggggtggggccaaggggaacacggaatggtcgcgcacgcgccggacgcagagccggataagcgtgtcccagagactggaccgtgtgcgtcaagccgcaaggcaaaggaagaaggagaagttcacggcgttgtaccacttgatcgacctcgaccttctggcgacggcgttcttctggctgaagcggaaggcggcagccggggtcgatggcgtgacgtggcacgactatgagcaagatctcgaccgaaatctcgaagacctgcatggcagactccgccggcaggcttatcgggcgctgcccagtcgcagacggtacataccgaaggcggatggcaaacagcgaccgctgggcattgctgcgctggaagacaagatcgtccagcgtgcactggtggcagtactcaatgctgtctacgagatggacttcttgggattctcatacggtttccggccgcaacgcagccagcacgatgcgctggatgcacttgcgacgggaatcgctcgcacgagcgtgagctggattctggacgccgacatcagcaggttctttgatacggtcgaccacgattggctgatccgtttcgtggaacatcgcataggcgaccagcgtgtgatccgactgatccgcaaatggctgaaggcgggagcgatggaagacggggtgatcgagcccacagacgaggggacgccgcaagggtctgtcatctcgcctctgctggcgaacatctacctgcattacgtcttcgacctctgggcaaaccagtggcgcaagcgccacgccgaggggaacgtcgtaatcgtccgatacgccgacgacgtcgtggttggtttcgataaaccacacgatgcgaagcggttcaggcgggccatgcaacaacgactggagcagtttgggctgtcggtacacccggagaaaacccggctgatcgaattcgggcgctttgcggcccggaaccgggcaagccgagggctgggcaaacccgagacgttcaacttccttggattcacccacatcagtggccgtgcgaaagacggtaggttcatgctcatgcgtaagacgcggagcgaccgtcttcgaacggcgctgaagggaatcaaggatgagttgcggcacagatggcaccagtcgattcccgagcaaggggactggttacgccgcgtggttcaagggtacttcaactaccatgcggtgccgaccaacttcgctgcgttgcgagccttccgcgcgcgcgagatcgacctctggaggctggcgcttcggcgccgcagccaaaaggacgacaccacgtgggcaaagatgcaccggctcgcgaagcagtggatacccaaggctcgcatccttcacccctggccggtcgtgcggtttgacgccaaccacccgaggcaggagcccggtgcgcgaatcgcgcacgccgggatctgtgcggggggtgcccagtaatgggcattcctaccgcgacagtgtagcct

SEQ ID NO: 192 - DNA, 2449 bp, Bacteroides vulgatus
  source 1..2449: mol_type = genomic DNA
tcagaagttcgtgcgacgtgaagttgcgtaagtgattgtgtaacatcaagattacacctccagttccgtctggagtagcctacggacacgtgcattgggagtaatccttttgtacggagctgtcccgacaaggtagcctttccaaaaggagaagattgaaccgtgaggggaaatcaacggctgataacaccattcagcacgaggagcttggcttagcggtatggtcaacgaaagtgaactgtccgataaacatcgtaaggtataacgagccaaaggctgttgacaggctcggaccaaaaggtaagcagtcggctgtcctcttgaactttgggggcacacggacattagcactgccggtggaatagacaggacctaacccgcttatttatcacttatgtaaaacacggtaagcccgtatctctccagttcatcagagctggtaagcaagccgcaaggcaagcctacatggggtgtgggtataggatagcggaaaaagcgaatgcctccctgtaatggggaggatacggattgagccaatatcacgggttggtaaaggtaacatcatccgacacgaaagtgggcagacttccgcctatggtgacactttacaagatagtttacagaacttttaaagaaaggaaagcaaatgaacgcaaagaaattggcgtgtgcgcccgaagacatagacattcacgaactctggtcgaaaatcgactgggacaaatgtgagcgttttgtccaaaagctgcaagcacgtattgtaaaggctcaaagggaaggcagaaataacaaggtgaaagccctgcaatggatgcttacccactcgttctacgccaaagcattggcggtaaaacgagttacgactaacaaaggaaaatctacttccggcgttgacaagattacatggtcttctccattggcgaaagccaaagccatattcactctgaaaagacatggctacaagccacaacctttaaaacgtgtaaatatcaaaaagaagaacgggaaattacgcccactcggaataccaacgatgaaagaccgtgcgatgcaggcattgtacctaatggcacttgacccgatagcggaaaccactggcgacagccattcctacggattccgcagacatcgctgtacacatgatgccatcgagcaatgctacatcgttctttctcgttcggtcgctcccgaatggattcttgagggggacatcaaaggatgcttcgaccatatcagccatgcgtggttaatcaacaatatccctatggataaggagatactacgcaaatggctcgaatgcggatatgtgttcaatggagaactctttcctacggaagagggaaccccacaaggtggcataatatccccaactcttgcaaacatggcacttgacggactgcaagatttgttggaaaagagtgtcaagaaatatcaggtgaactacaaaaagatagttcctaagatacatcttgtacgatatgcagatgatttcattgtaacggcaaaggataaggaaacgatagaacaggttatacttccattagtacgtaagttccttgcggaaagaggtctgacactatcagaagaaaagacaaaaatcacacatataaatgagggatttgactttctcggtttcaatatccgaaagttccgcaataatacgctgctgacaactccatccaaagatgcgcaaaagaggttttgcgaaaaaatccgcaagacgatagaagccaacaagtgtgtaaaacagaagtcacttattatgatgctgaatccaatcataaaaggttggggtaattattataaatatggtacttctgcgaatgtattccaccgaatggattgggaaatcttcaagaaaatatggcaatgggcaagacgcagacatccgcagaaatgtaaaggatgggtaaaggataagtatttcagaacactaaatgggcactcgtggagatttgcggcagatatgggcaagaaagataagattgattatctcgaactgacctatctaccaaccatccaccatgagaaattcgtcaaggttcggcattatgccaatccgtatgacccatcagataaatcgtactacgaatggagagaaacctaccgaatgaagcaaacgctgaaaggcaggcaatctctaataaacatttggaaacgacagaataaagtatgtcctgtatgtggagagcgtattgacagggaaagaccttggagtatcactgaacaaatagtcagtggacgaaaggtaagaactcttgtacataccagttgtaaacgtaaaatgcaaagtagattatgaatatgaaagtcgaaaatgagatggctctaccgataacaaagtcggtaacggtcatgttgcttgagccgtatgcggggaaactcgcacgtacggttcttagaggggaaagctcccgtaagggggctgacctactcgacagttcgggcg

SEQ ID NO: 193 - DNA, 1930 bp, Chlorobium phaeobacteroides
  source 1..1930: mol_type = genomic DNA
tcaatttagggtgcgccgggagaccggtaagaggtaaagggtgcaagtccctgcagtgaaaggattagcgatccgcactggccccgagtcatgcgcgcataaccgcgaggatatgcgcgaagcattgacaggggtgcatataggccagccattgagcggcgaaaatgagcaaatttggggagcccatgccctcctaaaagcagaaggcaatacagacgaagtgcgataacgcgagcacaagactgtccccacgacgtcagagaacctggcatgtatgtaagcctcttacacgggaaccgggagatctccgataccacccatcgtcgttggcgggtcgcatcgggaaaacgtaatagttgaagccgatgatgtacggtacggagaagtcagacctgcccatactaccggagaagcgagcgaacaaagcgggtaagcccgtagcggagttcgtggagggaaggggtgggaacaagagaaatgcggaactgcaaagcatggcccggacacagggtcgggaagccgtgttccaagcgcagggtcgcatacgtgaagcagtaaccagaaatcgaggagaaaagctgacagcgcttctgcatcacatctccgtagactgcctgcgctggtcgtacttcgaactgaagaagaccgcagcaacgggagtcgatgggataacgtggaaagattacggggaaggactggaagagaaccttgcagacctgcaccggcgaatccacacgggagcgtaccgggcacagccatcgcgccggaagtacataccgaaggcaaatggccaacaacgaccgctcggcatcgccgcattggaagacaaaattgtacagcgggcagtggtggcgattctcacgccaatctacgaagcggagtttctggggttcagctatggattccgtccgggacgcagtcagcatgatgcgctggatgcactcgcatacgggatcaaggtgaagaaaatcggctgggtattggacgccgatatttcccggttttttgatacgatcagccatgagtggatgatacgctttttggaacaccgtatcggtgacaagcggattgtccgactgattatcaagtggctgaaagctggggtactggaagatagcgtacggatcgaggcagaagaaggaacaccgcagggagccgtgatatcaccgctccttgcgaacatctaccttcactatgcttatgacctgtgggctaagcagtggcgggaaaagcactgcaaaggtgatatgatcgtcgtgcgtttcgctgacgatagtgtcgcaggcttccagaacaaggaagatggcgaaagattcctagctgatctgaaagagcgactggcaaagttcgcgctaacgttgcatccagagaaaacccgactgattgagttcggcaggtatgctgccaagaaccgacaaagacggggacaagggcggccggaaacctttgactttctcggatttacccacatctgtggagagaaagtagtggggaaaggctttcagctcttgcgcaagacgaagcgcggatcagtgagaaccaagctcggagaggtaaagaaagagctgagacggcgaatgcatgtctcagtatcggaacagggaaagtggctgaacagcgtgttgagaggtcactatgcctatttcgcagtgcccacaaacacgagggcgttatcggcattccgctaccatgttgccagacgttggatgaagagcctcaggcgccgaagccagcggcatgtgatgacatgggagcgaatgatgatatatattgatcagtacctcccaaacccaaagatccttcacccatggccggaacaacggttttgcgtcaaacactcaagttaggagccggatgcgggaattccgcttgtccggatctgtccggggggtgaggggtaactcttgtccctaccgggattttaaatatg

SEQ ID NO: 194 - DNA, 1893 bp, Coprococcus catus
  source 1..1893: mol_type = genomic DNA
aatcggaattgtgcgcccagataagggcgagtagtctagcaggtggaatgcctgtctggtaaggtctaaccaaccgccagtagcgagtcttgggtgactgtcagtaacggtagttgctaagcgtagacagcgagaaaatagagagtgtgattgagcctcgaaattttgacatttagaaggctgacgttctaatttcgacggaaagcaatacaaatcaattcgcattggtgagaaatggtttgcttctgcggggtcaaagagcactttatgttttacattgtgattattcagtcaactggggagagcctattgtttcttgccattgcaagtatggcaaacaactgcaaaacaggagaatgtcaaaagaacagtaggcagtcggatagcttcatagtactgaagaagttgggtaatgccaacggaggaaagggagctacataataatggtctttctgaggacacatcaaccgtactcagggacggaggtattgatggaaacgaaattagaaagaatagcagaaatatcggcaaattcgccgagaccagagttcacatcgttgtatcatctaatcaataaagaaatgcttttgcaatgtcacaaagaattagacggaaacaaagcagtcggtgttgatgagattactaagaaagagtatgagagaaatctggagcaaaacatagatgacttggtagaaagactgaaaagaaaatcatacaaaccacagccatcaattagggtatatatccctaaaagcaatggaaaacttcgaccattaggaattgcgtgctatgaggacaaaatagtccaattagcattaaagaagatattggaagctatctatgaaccgagatttctgaactgcatgtacggattcagaccaaaccgaggatgtcataacgcaataaaagaactgtataagagattaaacaacacgaaaatctgctatattgtggatgctgacatcaaaggattcttcgaccatatgaagcatgaatggattatcaagtttctcaaactttatattaaagaccccaatataattggtcttgtcaagaagtatcttaaagtgggagtgatggataatggtgaacttatggtgaatgaagaaggttcagctcaaggcaatatcataagtccaatcctagcaaacatatatatgcacaatgtattgacactatggtataagtttattattaccaaagaatgcaagggagataactttctaattgcatatgcagatgattttgttgcaggatttcaatgcaaatgggaagcagagaattactacaagcttcttaaagaacgaatggaaaagttcggtttacaattagaagatagtaaaagtcgtctcttacaaagcggtgcttatattgcaagggcaaagcaaaagagcggtgagtgtatcagattacaaacatttgattttctgggattcacattctattgtgggcgttcacgcaaaggcatgccatatataatgccaaagacaagctctaagaaattcagacagaaaattcgaggtatcaaggtatggttatatgcgaatagagaccaaccgctaaagaaactaatgggtatgctaaatctgaaactaataggtcattataggtactatggcataagttttaatggcagaatgatttcaaactacaaacaacaagtgagagaacttctgtttaaggtgctgaatagacgaagtgacagaaagagttatacaagagaaggattcattgaaatgctgaaatattatccattggcaatgccaaagatttatgtcagcttgttttgagaatgtaaatattattatgaagagccgtatgcgggaaagccgcacgtacggttctgtgaggggcttacattgtgaggtgtaagtctactcgactgaggtgatg

SEQ ID NO: 195 - DNA, 1898 bp, Cupriavidus taiwanensis
  source 1..1898: mol_type = genomic DNA
ttttctttttgtgcgcccggcagggcgcactcacttgagggtgaaagtcccttacacacccggcaaggggaagtgttagccataggcaagggttccccgggcgactgggggtctgaaggaagcccgaggcaaagcgctggcctgacgaacaggaagcggatatgaggcgacgcatctgggtaaggcagcaaaaagggccaaagcccagtacttgcacggaacggtgcggcgtagatccgacaggcataagcgtgaaggtgggtgcgtcatacccggggagatctgtaccgctgccacggtgctaccggcgtcgagaggtgtcgggatggcggtgcagaagtcagcagaggccatattaggcgagccgttggcgctgaagggccgaacatgaatgagcgcgattaggacgacagacctcgatgcttaccgacgaagcccaaatgcacgaacgagtgcagcccacagcggaggaaggcgggcggaacctgcccggggccgatgggggtgcggaggctggcacggcggctgttgggcaaacgaaagcgcgggcgccatcgctgatggaggcggtggtcgagagaagcaacatgtggctggcgtaccggagggtggtcggcaatggcggtgcggccggggtggatgccttggaggtgacggcgttgcgtgactggctgaaggtgagctggccaagcgtcagggcggcgttgctgggtggtcagtacatcccgcagtcggtgcgcgcggtggacattcccaagccttcgggcggggtgaggacactgggcatcccgacggtggtggaccggctgatccagcaggcgctgctgcaggttctccaaccgctctacgaaccggggttctccgagtcgagctatggcttcaggccgaggcgcagcgcccagcaggcggtcttgcaggcacagcggtacgtgcaggaaggccggcgatgggtggtggacatcgaccttgagaagttcttcgaccgggtcaaccacgacatcctgatgtcgcgggtggcacggcaagtgaaggatgtccgggtgctcaagctgatccggcggtatctggaagcggggctaatgcgtggcggggtggtcgaggcgaggaggcagggcacgccgcagggcgggccgctgtcgccgctgctgtcgaacattctgctgacggattgggaccgtgaactcgagaagcgggggttggcgttctgtcgctacgcggacgactgcaatatctacgtccgaagccaggcagcaggacagaggctgttggccgggatgatgacgttccttgcggagcggctgaatctgcaggtcaatgaggccaagagtgcctgtgcgcggccttgggcacgcaagttcctgggctacagcctgacagcgcatcgtcaggccaagctgcgtattgccccggaaagtctgcaaagactgaccggacgaatcaaggaactgatgcgcaaagggcgaggaaggagcctggctcacacgatagcggtgctgaatccggtgctgcgcgggtggatcgggtacttccagcacacccagagcaagcggccattggaggagctggacgggtggatacggcgccggctgcggtgcttgctatggcggcaggccaagagccgtgctacccgcactgtgatgctgcgtcggcaaggcttaacggaggatcgcgcgtggcactcggctcgcaacgggcagggcccatggtggaatgccggggctcgccacatgggcgcggccttcccgaagggctacttcgacgccttggggttgatctcgctgctggatactcagcagcgcttgcagtctcgttcgtgaaccgccgtatgcggaaccgcacgtacggtggtgtgagagggctgagggggtgacccctcaccctactcaatcctgccgcac

SEQ ID NO: 196 - DNA, 2179 bp, Chlorobium luteolum
  source 1..2179: mol_type = genomic DNA
atgacatggagtgcgacgtgaagtcgcgtatttgaactgttctttgcagaaccagttgtgccgtcaactgcccccaccggcacatgcaggatgagcaatcgccctgtgtgaagctaccggaaacgtacccgcgggaaaccgtaggccgacccgtgaggagaagctgaatctgccagtagcaggcggagaggggcaagggggaggatggtatggttaacgcaggtgaatcgtcacaaccatcgttataataagcaagcgaaagctcactgacaacgcttggaccaaaaggtacgcgggcaggtatgctcccttccatgtggagcacacacggccaacaaatcccgccggtggagagacggaacctaacccatccgcatgttctatacgcggaacacggaaaccccgtacttccgcccggccatgccgggcaatccgactgtaaggaaggaccatgaaagtgcgggtatgagagtggggaagaagcgaacgccgtcctgtaatcgggcggataggggttgcaacatcaccccacgtgaaaacgggctgacgtcctcatggtctcgatttgcaagataacttgaagaacctcccatgcagaaggaacgcaaatgaacacggataaccctgtgtgtgcaccttccggcctcgactggcaagggatcaactggtcccggatcaagcgacaggtcaggaggcttcaggcacgtattgcaaaggcaacaaaggaaggccgtcatggcagggtgaaagccttgcagtggctgctgacccactcgcacagcggcaaggttcttgccgtcaaacgggtgacggaaaaccgggggaaaaacaccccgggagtcgacggagacgtctggaaaacatcaaaagcaaaggccaatgccgcagcatcgctgagacgaagaggctacaagccccttccccttcgaaggacctacattccgaagaagaacggtaagcaacgacctctcggcatcccaaccatgaaagacagggccatgcaagcgctctactggcttgcactggagcctgtagcggaaaccaccgcggacggcaactcgtacgggttccgaccatggcggtcaacagccgacgtggcagaacagtgtttcatctgcctcgcaagacgcgattccgcacaatggatacttgaggccgatattgccgggtgcttcgatgctatcagccaccaatggctggtcgacaacatccccatggatacgccgatcctccgcaaatggctgaaggcaggcttcgtgttcaacaacgagctctttcccacagcctcgggcacgccgcaaggcggcatcatcagtccgggactggcaaacatgagccttgacggcctcgagcaagcgcttgcaacagcatttccgcaagcgcgaagacgaggactgaaaatgcatatggtgcgctacgctgacgacttcatcatcactggaaactcgaaagagtggctggaacatgagatcatgccggtagtggtcgacttccttaaaaaacgagggctctggctctcagaggaaaagaccagggtaacgcacattacggaaggatttgatttcctcggctggaacatgcgcaagtacgacggcaagctacttatcaagccctcgaaagcgaacatcaaggcccacctcaccaaggtgcggggaatcatcaaggcgaaaaagacgatcaagcaggtggatctcatcggtctcctcaaccccgtcctgcggggatgggcgaactaccaccggcacagtgtcgccaaagacgtcttcgctcgcaacgaccacgaggtctggtcaatgctctggaaatgggcgaaacgacgacacccaaataaaggccttcgatggatcatggacaaatactttcatgccagaggaggcaggaaatgggtgttcgtcgccgaagaggcggaccgaaagaaagaacgacggctctttctggaagccagcatgccaatccagcgacacgtcaaaatcagaacgaaggcaaacccgcacgatccagtgtggagggagtacttcagcgcccgtagaacacagatgtggaaagcgacaccattgagttgccgggtgccttacggcgccttattcgaggcttgagccgtgtgcggtgaaagtcgcacgcacggttcttaggggacggtggcgcagtaatgcgccgctgttacccgattatggaaaac

SEQ ID NO: 197 - DNA, 2175 bp, Chlorobium phaeobacteroides
  source 1..2175: mol_type = genomic DNA
caacccgttcgtgcgacgtgaaagtcgcatagttgaactgttctttgaaccagttgtgccgtcaactgctcccaccggcacatgcaggatgagtaatcgccctgtgtgaagctgccggaaacgtacccgcaggaaaccgtaggctgctccgcgaggaaatgccgaatctgccagtagcaggcggagaggggcaagggagagagtggtcttggctaaggtatctgaatcgctgcaaccgtcgttatgtcgaacgagtgaaagctactgtaacactcgaaccaaaaaggtatgcgagtaggtaaacgcctggatggagcgtttacgcggccaattactctcgccggcggctaggcggagcctacaccacttgagagttcactatgcggaacacggtaagcccgtagtgtcgcccagcatactgggcaatcgaaccgtaaggaacgaccatgatactgtgggtaaaggatcgaggagaaagcgaacgcctccctgtaatcgggcggataagggctacaatatcgccctatgcgaaagcaggcagacttcctcggggtctcgctttacaagaaaacttgaagaacctcttatgcagaaggaattgcaaatgaacatgggtagtaccgtgtgtgcaccttccgcctccaactggcaagggatcgaatggtctcaggtcaaacggcaggtcaagaggcttcaggcccgtattgtaaaggcaacacaggaaggtcgccactgcaaagtaaaagcattgcagtggttactgactcactcgtttagcggcaaagctcttgccgtgaagcgtgtgacggaaaaccggggaaaacacaccccgggagttgataaccaaatctggataacgccaaaagcaaaaaccaatgctgtagcatcactaaagcgaaggggttacaagcccctccctcttcgaaggatcaatattccgaagaagaacggtaagacccgacctctcggcatcccgacaatgaaagacagggccatgcaggcactttatctgcttgcactggaacctgtagcggaaaccactgcggacgataactcgtacgggtttcggccatggcggtcaacagcagacgcgagtgcaagatgttttacctgcctcgctcaacgcaattccgcccaatgggtacttgaggctgatattgccagttgcttcgatgctataagccatgagtggttgatcgacaacattccagttgacactgcgatcctccgcctgtggctaaaagcaggatttgtgctaaagaacgagctctttcccacagaagccggtacgccgcaaggcggaatcatcagcccggttctggcaaacatgtgtcttgacggtctcgagaaggcgctggcgaaagcctttccgcaagcaaagaaacgcggcctgaaaatgcacatggtgcgatacgctgacgactttgttataacaggaaactcgaaagagttgctggaaaacgaggtcttgccagtggtggtcgaattcttggctgaaagagggttattcctctctccggaaaagactaagataacgcacattacggaaggctttgatttcctcggctggaatgtacgcaagtacagcggcaaactgcttatcaagccgtcgaaagtgaataccaaggcccacctcctgaaggtgcgggaaatcattaaaggtaataaaacggccaaacaggtaagcttgatctacctcctcaatcccataatgaggggatgggcgaactaccatcaacatgtggtcgccaagaaatcctttgctcgcaatgacgcagaaatctggtcaatgctctggaaatgggcaaaaagacgacacccaaacaaaggaatgcgatgggtcaaggcacggtacttcaagacccaaaacgcccgcaactgggtgtttgccgctaaggatgaggccacaggcaaggaaatcagacttgtcactgaagccgatacaccaatcaagcgacatgtcaaaattaagtcaaaggcgaaccctcacaatccagtgtgggccgaatactttgcagcccgcaacaaacagatgaagaaatcagcaccgatgtacagccgggcgtcacaaggcgccttatgcgaggcttgagcccagtgcggtgaaagtcgcacgctgggttcttaggggacggtggcgtagcaatgcgccgccgttacccgacatcgagtcgt

SEQ ID NO: 198 - DNA, 2093 bp, Geobacter uraniireducens
  source 1..2093: mol_type = genomic DNA
tgtttattcggtacgccaggccaagcctggctgatctatctgactgaaaagtgtcagacatgtaaattgctcgttcaccatgtagtcgcggtctgtcacgagagggtaaccaaacgtggggagtaagaccggttctttgtaagaaaagagcgaagggtacgataactaccctgacgacgaagacgcgagcccagcgcaagcgaacccgtttcggcctcgttacacgtagttgacagtggccaaccttccgaatgtggtgaagcctgccatccaccggtgataaacgagcgaaagaaccggtgggactgtccggggtgattggggagggcgcgtgtcgaaagatcagccgaggaacctgggaggtccgacgtaattcccaaagcgaaagggtttcgccgaagggagaacgccgacggggaatcaataacccgccaagcgtgtacggtcggaattcggagcggcccatagtagccgggaagcggggtaatgcccgtggagccaaggggccgtacttcagtcatgtttacacggaaagagaggagacccgcttgagcgacagacgctccataacggaatggatggccgaaggattcgaacctgaacccggactgccggtaaaagtctcccttctgcggtggaaactgagcagcaaggccaaacgagaaccgcagttccggttctatgccctttacgaccgaatccatcgtcgtgatgtacttgaaacggcgtggttgcgagtaaaggcgaacaagggcgcgccgggagtcgatggtgtcagcattgaatccatcgaggtgcgggcagacggaatatccggctatcttgacgaaattcaagaaagcctgagaaccaaaaactacaaaccatccccggtgcgtcgcgtctacatcaccaagcccaacggcaaactgcgcccgctcggcataccgtgcgtgcgggacagaattgtacaggcagccgtactccttatcctcgaaccgatatttgaggtggactttctcgactgctcccatggatttcgtcccaaaaggcgtccccatggagccctcgaccaagtcggtaataacctccaactcggacggcaggaagtgtatgacgcggatctttcgagctactttgacagcatcccccacgagcacctgatagtagaacttgagcggcgcatagccgaccggagtgtcctcaaactgatccggcaatggcttcacagtccggtcagagaagaagacggcagcatcagccgtcccaaacaagggacgccacaaggcggggtaatatctccccttcttgccaacatctaccttcaccggctcgaccgggcctttcacgaagaagcagacagcccctaccacttcgccagagcgcgcatggtacggtttgccgacgacttcgtagtaatggccagacacatgggcaatcgcataaccggatggctggaagagaaactcgaaacagaccttggactcagcatcaaccgtgacaaaaccggtattgtcagaatgaacaagaaagagagcctgaactttctgggattcaccctccgctatgaccgtgacctgcggggcagagattgggattatctcaacatcatgccgagtaagaaggccataggagcactcaagggaaaaatccgggagaagacccgaagcggatacaaaaagccgcttgtcgaagcaattgccgaggtaaacagcatccttcgcggatggggcaactacttcgactacgggtatccccggaaagtatttcgagacgtgaatcattacacgaggactcgctttcgaatatttctgaacaatcggagccaaaggcggagcaaacccttcagggcaggtgaaagcctgtatgccggacttaagcgatatggtctggtttacctgtaacagcaaagccacaactcctcaataatgccgtgcagcaagaggctgataggtaagccgtatgcgggaaatccgcacgtacggtttgacgagggggagctggttaaggcaactatggtaaggctacttaggcaccgccaaacgaaaggggcggaaacggacaagccaaacctaatgatgtcgtaacctgcttcctactctactggttaattg

SEQ ID NO: 199 - DNA, 1908 bp, Geobacillus sp.
  source 1..1908: mol_type = genomic DNA
ctcttttcatgtgcgcccggcatgggtgtagtctatagggtgaaagtcccgaactgcgaaggcagaagtagcagttagcttaacgcaagggtgtctgcggcgacgcagaatctgaaggaagcgggcggcaaacttccggtctgaggaacacgaacttcataggaggctgggtatcattgggtgagtttgcatgacaaaacgaagccctttctgccgaaggtgataccgagtaaatgaagcagatagatggaaggaaagactgcactcttacccggggaggtctgtccgggaagccaagtgcgcttggcaaccgttggagcgatccaacgctgaacggacagaagtcagcagaggtcatagtactcgtctagcttaagatagaaggggaaggaccgaaccatgaaggagaacggccactaggcgttcatcttctttgatgaaacagacaacccgaaagggcctgcttgagggaggaaatggtgaagtccatgggggacctcaagagggtggagaagaagatggcacgaatagaacggatcgttcacgtagagaggaagaattggaatgtggatgaaacagatactgtcacgggagaatctcctgcgagcactcaaacaagtggaaaagaataaagggtcccatggaaccgatggaatgtccgtcaaagacctgcgaagacacctcgtggaacattgggacgtgatacggcgtgctttggaagaagggacctacgaaccttgcccggtccgacgggtcgaaatcccgaaaccgaacggaggagtcaggttactaggaatcccgaccgtgacagaccggttcatccaacaggccatcgcccaagtgctcacgccgatctttgacccatccttttcggaacacagctacgggtttcgtcccggtcgaagaggacacgacgcggtgaaaaaggcgaagcagtatattcaggaaggatatacatgggtggtagatatcgacttggaaaagttctttgatcgagtcaaccatgacaaactgatggggatattagcgaaacgaattccagacaaaatcctcctaaagttgatacggaagtatttacaggcaggggtcatgatcaacggggtggtcatggaaacacaagaggggactccacaaggagggccgctcagtccacttttgtccaacattctcttggatgagctggacaaagaattggaaaaacgagggcacaaatttgtacggtatgcggatgactgcaatatctacgtaaggacgaagaaggccggggaacgggtgatgaaatcgatcacggcattcattgaaaagaaactccggctgaaagtcaacgaaaccaaatcggcagtggatcgaccgtggaggagaaaattcctcggttttagcttcaccccaaataaggagccaaaaatccggatcgcaaaggaaagtattcggcgcatgaagcaaaggatgcgcaccatgacgagtcgatcgaaaccgattcccatgctcgagcgaatcgaacagctcaaccagtacattctgggatggtgtggatacttctcgttagcagagactccaagtgtgttcaaagaactagatggatggattcaacgaaggctgcgcatgtgccaatggaaagagtggaaacttccgagaaccagagtccgaaaactgcaaagtttaggagtgcccaagcggaaagcatatgaatggggaaacactcggaagaaatattggagagtggccgctagtcccatcttgcataaagcccttggcaactcctattgggagagccaagggctgaagagtctttatcaacgatatgaatctctgcgtcagacttaatggaaccgccgtataccgaacggtacgtacggtggtgtgagaggacgagggttaatcaccctctcctactcgatcattctgttg

SEQ ID NO: 200 - DNA, 747 bp, synthetic construct
  source 1..747: mol_type = other DNA
ttaaaacagctctggggttgttcccaccccagaggcccacgtggcggccagtactccggtattacggtacccttgtacgcctgttttatactcccttcccctgtaacttagaagcatacaaaccaagttcaatagaagggggtacaaaccagtaccaccacgaacaagcactcctgtttccccggtgacattgcatagactgtacccacggttgaaagcgatcgatccgttacccgctcctgtacttcgagaagcctagtatcatcttggaatcttcgatgcgttgcgctcagcactcaaccccagagtgtagcttaggctgatgagtctggacgtcccccaccggcgacggtggtccaggctgcgttggcggcctacctgtggcccaaagccacaggacgctagttgtgaacaaggtgtgaagagcctattgagctacaagagagtcctccggcccctgaatgcggctaatcctaaccacggagcaggcagttgcaaaccagcaaccggcctgtcgtaacgcgcaagtctgtggcggaaccgactactttgggtgtccgtgtttccttttatttttacaatggctgcttatggtgacaatcatagattgttatcataaagcgacttggattggccatccggtgaaagtaaaacacattgtttacttgtttgttggattcactccaattaacacttttacttacaaactcattacaacaactctattaattagagataagcatcaca

SEQ ID NO: 201 - DNA, 612 bp, synthetic construct
  source 1..612: mol_type = other DNA
tttcccctgttcgtaactaagtgtgtgcccaatctcctcactcctgctggcttcaccgaccggcagtgtccaaaatgctaggtgaatcccctccctttcctctgggcttctgcccagcttcctccccccagcctgacgtgacacaggctgtgcaaagaccccgcgaaagctgccaaaagtggcaattgtgggtcccccctttgtaaaggcgtcgagtctttctccctcaaggctagacccgtcagtgaattctgtcgggcaactagtgacgccactgcacgcctctgacctcggccgcggagtgctgccccccaagtcgtgcccctgaccacaagttgtgctgtctggcaaacattgtctgtgagaatgttccgctgtggctgccaagcctggcaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggttcgccagggatctgagcgtaggctaattgtctaagggtattttcatttcccattctttctttcttgttcata

SEQ ID NO: 202 - DNA, 439 bp, synthetic construct
  source 1..439: mol_type = other DNA
tccccggcatgagaggaatagactctttcagggttgaagccacgagtgtcgttacccgcactggtactacgcaaagcctagtaacatcttgaaactctttttggttggtcgttccactagttaccccctagtagacctggcagatgaggcaggacgctccccactggcgacagtggtcctgcctgcgtggctgcctgcacacccttcggggtgtgaagccaaaagaaagacaaggtgtgaagagccccgtgtgctaccagtgaatcctccggcccctgaatgcggctaatcttaccccacagctattgcacacaatccagtgtgtatgtagtcgtaatgagcaattgtgggacggaaccgactactttgggtgtccgtgtttccttttattcccatgtttctgcttatggtgacaatactgacgtatagtgttgttacc

SEQ ID NO: 203 - DNA, 627 bp, synthetic construct
  source 1..627: mol_type = other DNA
ttaaaacagcggatgggtatcccaccatccggcccactgggtgtagtactctggtacattgtacctttgtacgcctgttttccccctcttgtacccgcccttcaagctccttgcccaagtaacgttagaagtttgaacattggtacaataggaagcatcacatccagtggtgtactgtacaaacacttctgttgccccggagcgaggtatagatggtccccaccgtcaaaagcctttaaccgttatccgccaatcaactacgtaatggctagtagcaccttggatttaagttggcgttcgatcaggtggtaacccccactagtttggtcgatgaggctaggaattccccacgggtgaccgtgtcctagcctgcgtggcggccaacccagcatccgctgggacgccaatttaatgacatggtgtgaagacctgcatgtgcttgattgtgagtcctccggcccctgaatgcggctaaccctaaccccggagccttgcagcacaatccagtgttgttaaggtcgtaatgagcaattctgggatgggaccgactactttgggtgtccgtgtttcttatttttcttgaatttttcttatggtcacagcatatatacattatatactgtgatc

SEQ ID NO: 204 - DNA, 737 bp, synthetic construct
  source 1..737: mol_type = other DNA
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 205 - DNA, 821 bp, synthetic construct
  source 1..821: mol_type = other DNA
ttcaaacagcctgggggttgtacccacccctggggcccacgtggcgctagtactctggtacgttagtacctttgtacgcctgttttcccctcccttaaacaaattaagattaccactactgaggggagtagtccgactccgctccggtactgccgcaccagtactccggtacacttagtaccctagtacggagtagatggtatccccaccccgcaacttagaagcatgcaaacaaaccgaccaataggcgcacgatatccagtcgtgtttcggtcaagcacttctgtctccccggtccgaaaggatcgttacccgcccgacccactacgagaagcccagtaactggccaagtgattgcgaagttgcgctcagccacaaccccagtggtagctctggaagatggggctcgcgtctcccccgtggtgacacggtcgcttgcccgcgtgtgcttccgggttcggcctacgccgttcacttcaatgtcacgtaaccagccaagagcctattgtgctgggacggttttcctccggggccgtgaatgctgctaatcccaacctccgagcgtgtgcgcacaacccagtgttgctacgtcgtaatgcgtaagttggaggcggaacagactactttcggtaccccgtgtttcctttaaattttattcattattttatggtgacaattgctgagatctgcgaattagcgactctgccgttgaatattgctctgtactatttggttgcattccacaaaacctctgacatccccagtacatacattactttacttgtttacctcaatctaaagcacaagctagataatacaaa

SEQ ID NO: 206 - DNA, 822 bp, synthetic construct
  source 1..822: mol_type = other DNA
tttaaacagcctgggggttgttcccacccctggggcccacgtggcgctagtactctggtacgctagtacctttgtacgcctgtttttcccctcccttaaataaatcaaggttgccactactgaggggagtagtccgactccgctccagcaatgctgcaccagtgcactggtacgctagtaccttttcacggagtagatggtatcccttaccccggaacctagaagattgcacacaaaccgaccaataggcgcaccgcatccagccgtgcagcggtcaagcacttctgtctccccggtctgtaaagatcgttatccgcccgacccactacgaaaagcctagtaactggccaagtgaacgcgaagttgcgctccgccacaaccccagtggtagctctggaagatggggctcgcaccacccccgtggtaacacggttgcctgcccgcgtgtgcttccgggttcggtctcgtgccgttcacttcaacttcacgcaaccagccaagagcctattgtgctgggacggttttcctccggggccgtgaatgctgctaatcccaacctccgagcgtgtgcgcacaatccagtgttgctacgtcgtaacgcgtaagttggaggcggaacagactactttcggtaccccgtgtttcctctcattttatttaatattttatggtgacaattgttgagatttgcgctcttgcaacgttgccattgaatattggcttatactatttggttgccttttacaaaacctctgatatacccagttcttacattgatctgcttgtttttctcaatttgaagtatagactacaaatagcaaa

SEQ ID NO: 207 - DNA, 830 bp, synthetic construct
  source 1..830: mol_type = other DNA
cccccctcccccccttcccttccctttgcaacgcaacaattgtaagtgccctcacctgtcaattgggaccaccactttcagtgaccccatgcgaagtgctgagagaaaggaagctttcttacccttcatttgtgaacccactggtctaagccgcttggaatacgatgagtggaaaagttcattcttaatggagtgaaacatgcttaaatttccagctcgtgctggtctttccagtacggggcggccctgtctggccgtaattcttcagagtgtcacgccacacttgtggatctcacgtgccacatgacagcgctacagctggaactgggtgcttggtgcccatggagtaacagcgaaaagtgttagatcaagccttgcttgggctatgagcctgcggaacaacaactggtaacagttgcctcaggggccgaaagccacggtgttaacagcaccctcatagtttgatccacctcagggtggtgatgtttagcagttagtagttgccaatctgtgttcactgaaatctcggcataccgtgtagtgtacaggggtgaaggatgcccagaaggtacccgtaggtaaccttaagagactatggatctgatctggggccttgtccggagtgctttacacacggctcaaggttaaaaaacgtctagccccacagagcccgagggattcgggttttccctttaaaaacccgactagagcttatggtgacaattattgctgttcagacgaacagtgtaattgttgtctattcacagcagttctatcagagcttttcccacaacggatcttcttggcaagcaaatacagcaggagtcaat

SEQ ID NO: 208 - DNA, 1000 bp, synthetic construct
  source 1..1000: mol_type = other DNA
cactacgttacggttcccgcccgggacaactggtaccccattaggctacaacatggctgaaaagggtattgggtccccccggattgtgtccgttcgtagtgtgtgtaacgtggtttaccatctccactaacattggactaagcatttcatctttcctccccgattgtgtactcacttggctaacgctgggtggtcgcggttgggtccttgatttactttttctcgtctaagcattccgactgtcctccccgattatgtgctcattcagttaactgctgggtggtcatgactaacatcgaggaaccttctgtccacgcttactttgagctccggtcgcttgacgcttgtagggcgatagggttatcttcctgacaacatctttattctacctccataggctctatctatggagacggagtgtggcacccgtcccttctttgggagcttcggtagtgacgccctttgtcactctcgccagccgaggcatgcctggtgccaggtagcaaagaaagcatatgtttaaggacttgactgatttagcgcaagagtttgtagcgatgtccatagtgtctgcggattccccacacggcgacgtgtgccgcggaggccaaaagccacggtgttcacagcacccctatggatgcccacagaccccagtgggcactcttgttgccggactttcaggaaattaggcataggctcttctcaaactcctggcattggactaggtaagaatgccccggaggtaccccagtactccttcgggagtctgggatctgaccgggggccccacaaacatgctttacgtgtttcgtgcggtcaaaaattgtctaactagtcccaaccttgaacaagggattgttctttcctttttattactgagactggcctatggtgacaacagagattgactgtgaatacagttattttctggtgtttatcatttggtttttctccgtgctcttttacctttgtggtatttgttctttagataggcaaa

SEQ ID NO: 209 - DNA, 1000 bp, synthetic construct
  source 1..1000: mol_type = other DNA
cccggccaccccctttcgacgcgggtactgcgatagtgccaccccagtctttcctactcccgactcccgactctaacccaggttccttggaacaggaacaccaatatactcatcccctggatgctgactaatcagaggaacgtcagcattttccggcccaggctaagagaagtagataagttagattccaaattgatttatcatccccttgacgaattcgcgttggaaatgcacctctcacttgccgctcttcacacccattaacttgattcggcctctgtgttgagccccttgttgaagtgcttccctccatcgtgacgtggttggagatctaagtcaaccgactccgacgaaactaccatcatgcctccccgattatgtgatgctttctgccctgctgggtggagcatcctcgggttgagaaaaccttcttcctttttccttggaccccggtcccccggtctaagccgcttggaataagacagggttatcttcacctcttccttcttctacttcatagtgttctatactatgaaagggtatgtgtcgccccttccttctttggagaacacgcgcggcggtctttccgtctctcgaaaagcgcgtgtgcgacatgcagagaaccgtgaagaaagcagtttgcggactagctttagtgcccacaagaaaacagctgtagcgaccacacaaaggcagcggaccccccctcctggcaacaggagcctctgcggccaaaagccacgtggataagatccacctttgtgtgcggcacaaccccagtgccctggtttcttggtgacacttcagtgaaaacgcaaatggcgatctgaagcgcctctgtaggaaagccaagaatgtccaggaggtaccccttccctcgggaagggatctgacctggagacacatcacatgtgctttacacctgtgcttgtgtttaaaaattgtcacagctttcccaaaccaagtggtcttggttttcactctttaaactgatttcact

SEQ ID NO: 210 - DNA, 1000 bp, synthetic construct
  source 1..1000: mol_type = other DNA
cccccggttaccccctttcgacgcgggtactgcgatagtgccaccccagtctttcctactcccgactcccgaccctaacccaggttcctcggaacaggaacacaaatttactcatcccctggatgctgactaatcagaggaacgtcagcattttccggcccaggcttagagaagtagataagttagaatctaaattgatatgacttccccttgacgaattcacgttggaaatgcacccctcacttgccgctcttcacacccactaattgattcggcctactgtgttgagccccttgttgaagtgcttccctccctcgtgacgtggttggagaaatcttgtcacccgactccgacgaaactaccatcatgcctccccgattatgtgatgctttctgccctgctgggtggagtatcctcgggttgagaaatccttcttccttttaccttggaccttggtcccccggtctaagccgcttggaataagacagggttattttcacctcttcttcttctacttcatggtgctctataccatgaaagggtatgtgtcgccccttccttcttggagaactcacgcggcggtcttccgtctctcaaaaagcgcgagtgcgacatgcagagtaacgcgaagaaagcagttcctggcctagctctagtgcccacaagaaaacggctgtagcgaccacacaaaggcagcggaactcccctcctggtaacaggagcctctgcggccaaaagccacgtggattagatccacctttgtgtgcggtgcaaccccagcaccccggtttcttgttgacactctagtgaatccttgaatggcaatctcaagcgcctctgtaggaaagccaagaatgtccaggaggtaccccttcctcgcggaagggatctgacctggagacacatcacacgtgctttacacttgtgcttgtgtttaaaaattgtcacagctttcccaaaccaagtggtcttggttttccttttttatcctactgtcaat

SEQ ID NO: 211 - DNA, 415 bp, synthetic construct
  source 1..415: mol_type = other DNA
tcctcacccatgcttttcctacccccaccacgcccgcatgtttactgctttccttgatgctgcccgtgactacttccatgacctccccaacccaaacctcaaacgccttaagctatcatgggctctctatcaccaatccccttccttcccacccagaacccccccctccctcccctacaacgtgtatgagcaagacgagtttgacaagctcgccgaagccatgctcacccactctcccttccccacatcccattacctcttccaccccctcccgtccaacgcccgcaccatcgtcgagcgtgaggctgactgggatggctgtgatctagaaaagaaatggctcgacctcgtcatagaggacgatgccaagttccttctggagaacggctctctcccgtttggctccacccttgcc

SEQ ID NO: 212 - DNA, 659 bp, synthetic construct
  source 1..659: mol_type = other DNA
ttctcgcctgagtcaacaaagcgagaaacctgcccctccagcgccagacgagcggcataaaacttgaacttctggcatgctccaccacccttttccccattccaacccccattgcgctctcaaggtcgcgctttttcgagactagctcggattcaaaagttcctggcaccctttgccccttcaggcccttaaggtaggaactgaccttgtgctgtgccctcggtgcggaagtgctactgcgtagcgattgtaagatccctttgtggttctgccctggcaaggttatagagtactgtgatccgctgcggatgccatcctggtaacaggacccccagtgtgcgcaacagtatgttcacggtcttccgtgtccaccacattcggaacactgctctcgtgaaacagtgtgtgtccaatccctgcaatcagtatcaactacaccacctaggaatgctaggaaggtaccccggtccgccgggatctgatcctaggctaattgtctacggtggtgctcctttttattttccacttcaattcattggttacaactgctcgatccctgtgtttgctgcccttctctgctctcatcgccattctcaagtgttcacactgtccaagttcctttggttgttcgcttccacttgccactgtcaactcttgtc

SEQ ID NO: 213 - DNA, 711 bp, synthetic construct
  source 1..711: mol_type = other DNA
tcaccctcttttccggtggtccggacccagaccaccgttactccattcagcttcttcggaacctgtttggaggaattaaacgggcacccacccaccttcacccccttttcgtaactaagtgtgtgcccaatctcatgactcctgctgacttcaccgaccagcagtgtccaaaacgctaggtgaatttccttcctccccctctgggcttctgcccagctccctccctccagcctgacgtgccacaggctgtgcaaagaccccgcgaaagctgccaaaagtggcaattgtgggtcccccctttgtaaaggcgtcgagtctttctcccttaaggctagacccgtcagtgaattctgtcgggcaactagtgacgccactgcatgcctccgacctcggccgcggagtgctgccccccaagtcgtgcccctgactacaagttgtgctgtctggcaaacattgtctgtgagaatgttccgctgtggctgccaagcctggtaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggctccccagggatctgagcgtaggctaattgtctaagggtattttcatttcccactctttctttcttgttcata

SEQ ID NO: 214 - DNA, 650 bp, synthetic construct
  source 1..650: mol_type = other DNA
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 215 - DNA, 625 bp, synthetic construct
  source 1..625: mol_type = other DNA
attctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 216 - DNA, 549 bp, synthetic construct
  source 1..549: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacg

SEQ ID NO: 217 - DNA, 675 bp, synthetic construct
  source 1..675: mol_type = other DNA
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttttattgcgtttattgagagatttccaacaattgaacagactagaaccacttgttttatcaaaccctcacagaataagataaca

SEQ ID NO: 218 - DNA, 549 bp, synthetic construct
  source 1..549: mol_type = other DNA
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgcgtacaactcactgactatccacttgctctgtcacg

SEQ ID NO: 219 - DNA, 757 bp, synthetic construct
  source 1..757: mol_type = other DNA
cactatttagagaaagtaggagtctggattcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtc

SEQ ID NO: 220 - DNA, 767 bp, synthetic construct
  source 1..767: mol_type = other DNA
caacaggacatacgatgccgacagtaaaacagctctggggttgttcccaccccagaggcccacgtggcggctagtactctggtatcgcggtacctttgtacgcctgttttatacccctaccccatgtaactttagaagcagttcaacaagttcaatagggggggtgcaaaccagcaccaccacgaacaagcacttctgtaaccccggtgaccttgtataagctgtacccacggctgaaagcgagtgatccgttatccgcttaagtacttcgagaagcctagtatcaccttagaatcttcgatgcgttgcgctcagcactctatcccgagtgtagcttaggctgatgagtctgggcgttcctcaccggcgacggtggcccaggctgcgttggcggcctacccatggctaacgccatgggacgctagatgtgaacaaggtgtgaagagcctattgagctacttaagagtcctccggcccctgaatgcggctaatcccaaccacggagcaggtgccttcaacccagagggtggcctgtcgtaacgcgcaagtctgtggcggaaccgactactttgggtgtccgtgtttccttttatcttttatatggctgcttatggtgacaatcatagattgttatcataaagcgacttggattggccatccggtaaaatacaaacacatcatttacttgtttgttggatttactccactctcccaatttactcctagcataatatccattgttttgttaacaagacattactatcaca

SEQ ID NO: 221 - DNA, 766 bp, synthetic construct
  source 1..766: mol_type = other DNA
gaacgtagcggctacactacttaaacaagcctgtgggttgttcccacccacagggcccactgggcgctagcacactgattctgcgggatctttgtgcgcctgttttataaccccttccctaagcagcaacttagaagcttcacactatcacgaccagcagtgggcgtgacgcaccagtcacgtcttggtcaagcacttctgtttccccggactgagtatcaatagactgctcatgcggttgaaggagaaaacgttcgttatccggctaactacttcgagaaacctagtagcaccgtgaaagttgcggagtgtttcgctcagcacttcccccgtgtagatcaggtcgatgagtcactgtaaaccccacgggcgaccgtgacagtggctgcgttggcggcctgcccatggggaaacccatgggacgctctaatacagacacggtgtgaagagtctattgagctagttagtagtcctccggcccctgaatgcggctaatcctaactgcggagcgcgtaccctcaacccagagggcggcgcgtcgtaatgggtaactctgcagcggaaccgactactttgggtgtccgtgtttcctttttattccttattggctgcttatggtgacaattgaaaagttgttaccatatagctattggattggccatccagtgaccaacagagctattgtttacctgtttattggatacgtccctcttaatctcaagaccatccaaactcttgattatatattactccttaatcataagaa

SEQ ID NO: 222 - DNA, 522 bp, synthetic construct
  source 1..522: mol_type = other DNA
cgagctccgtgaataaactccttcactttccataactacagtgctttggtaggtaagcatcctgaccccccgcggaagctgccaacgtggcaactgtggggatccaggcaggttatcaaaggcacccggtctttccgccttcaggagtatccctgccggtgaattccgacagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggtttcccggcgcatgtttggcgaaacgctgaggtaaggttggtgtgcccaatgcccgtaatttggtgacacctcaagaccacccaggaatgccagggaggtaccccacttcggtgggatctgaccctgggctaattgtctacggtggttcttcttgcttccacttctcttttttctggcatg

SEQ ID NO: 223 - DNA, 661 bp, synthetic construct
  source 1..661: mol_type = other DNA
aggagtgctgatatagattcggatttaggccgtggtgcccgtgggcacgacggccacggctccagccgcactcctctccacgccttgtcccacgggacataaagtggagccctaagccctaaacccccatgttacccaattttcttatccacttccctttttccctttgactcacatttttcttgaccgcgcttgtgaggcggttcccgtacagttgagttgacgggtcccgcgcgggacctgaaaattactccatactgattaggaaaggttctagtacgcttgtacccttaaggccagcgggctacctcctttaggtcttatagccccaactggttagaaccactccagctcacctctatcggtgcgggagtgctaccacccctgcctgcgtcaagcccgtttttctagtgatctgtccacaaacacttggattcccgtcgcgactgacagaacctggcgacaggtgtctcgatgtgtgtgaccaagtggcatgagaatcggaaactaacgctggtaagatggtaagtgcccaatccctgtggggtgatgtttttctctgcataggaatgctgcgaaggtaccccggccttgagtcgggatctgatcgcaggctaattgtcctctgtgcagttcatttccttttcttttcattggcatc

SEQ ID NO: 224 - DNA, 747 bp, synthetic construct
  source 1..747: mol_type = other DNA
gtccactgagacatcttactcacccactcacaggacccacgcggtgctagcactctggttccacgggacctttgtgcgcctgttttatgtcccctccccaatttgtaacttagaagcaatttacaacactgatcagtagcaggcatagcgcaccagctatgtcttgatcaagcacttctgtttccccggaccgagtatcaatagactgttcacacggttgaaggagaaagcgtccgttatccggctaactacttcgagaaacccagtagcaccattgaaactacagagtgtttcgctccacacttcccccgtgtagatcaggtcgatgagtcactgcaatccccacgggcgaccgtggcagtggctgcgctggcggcctgcctatggggcaacccataggacgctctaatgtggacatggtgtgaagagtctattgagctagttagtagtcctccggcccctgaatgcggctaatcctaactgcggagcacatgccttcaatccagagggtagtgtgtcgtaatgggcaactctgcagcggaaccgactactttgggtgtccgtgtttccttttattctcacattggctgcttatggtgacaattacagaattgttaccatatagctattggattggccatccggtgtgcaatagagctattatatacctatttgttggctttgtaccactgaccttaaaatctttaaccactcttgagtgtatattaacccttaatacaatcaaac

SEQ ID NO: 225 - DNA, 717 bp, synthetic construct
  source 1..717: mol_type = other DNA
tccgaggaacagcataaacataaaacagccttggggttgttcccactccaagggcccacgtggcggctagtactctggtacttcggtacctttgtacgcctgttttatctcccttcccaatgtaatttagaagctcttaaatcaaggctcaataggtggggcgcaaaccagcgctctcatgagcaagcactcctgtctccccggtgtggttgtataaactgttcccacggttgaaaacaacctatccgttatccgctatagtacttcgagaaacctagtatcacctttggattgttgacgcgttgcgctcagcacactaacccgtgtgtagcttgggtcgatgagtctggacacaccccactggcgacagtggtccaggctgcgttggcggcctactcatggtgaaaaccatgagacgctagacatgaacaaggtgtgaagagtctattgagctactatagagtcctccggcccctgaatgcggctaatcctaaccatggagcaagtgctcacaggccagtgagttgcttgtcgtaatgcgcaagtccgtggcggaaccgactactttgggtgtccgtgtttcactttttacttttatgactgcttatggtgacaatttgatattgttaccatttagcttgtcaaattaattgcaaaggatcctaaatcttacttaccaatttgcattttgacaatcttgatttgaaaattttaata

SEQ ID NO: 226 - DNA, 751 bp, synthetic construct
  source 1..751: mol_type = other DNA
ttaaaacagcctgtgggttgtgacccacccacagggcccactgggcgctagcactctgattctacggaatccttgtgcgcctgttttatgtcccttcccccaatcagtaacttagaagcattgcacctctttcgaccgttagcaggcgtggcgcaccagccatgtcttggtcaagcacttctgtttccccggaccgagtatcaatagactgctcacgcggttgagggagaaaacgtccgttacccggctaactacttcgagaagcctagtagcaccatgaaagttgcagagtgtttcgctcagcacttcccccgtgtagatcaggtcgatgagtcactgcgatccccacgggcgaccgtggcagtggctgcgttggcggcctgcctgtggggtaacccacaggacgctctaatatggacatggtgcaaagagtctattgagctagttagtactcctccggcccctgaatgcggctaatcctaactgcggagcacataccctcgacccagggggcagtgtgtcgtaacgggcaactctgcagcggaaccgactactttgggtgtccgtgtttccttttattcttatactggctgcttatggtgacaattgaaagattgttaccatatagctattggattggccatccggtgtgcaacagagctattatttacctatttgttgggtatataccactcacatccagaaaaaccctcgacacactagtatacattctttacttgaattctagaaa

SEQ ID NO: 227 - DNA, 747 bp, synthetic construct
  source 1..747: mol_type = other DNA
ttaaaacagctctggggttgttcccaccccagaggcccacgtggcggctagtactccggtatcacggtacccttgtacgcctgttttacactcccttcccccgtaacttagaagaaacaaacaaagttcaatagaagggggtacaaaccagtaccaccacgaacaagcacttctgtttccccggtgacgttgtatagactgtacccacggtcgaaaacgattgatccgttatccgcttttgtacttcgagaagcctagtatcatcttggaatcttcgatgcgttgcgctcagcactcaatcccagagtgtagcttaggtcgatgagtctggacgttcctcaccggcgacggtggtccaggctgcgttggcggcctacctgtggcccaaagccacaggacgctagttgtgaacaaggtgtgaagagcctattgagctacaagagagtcctccggcccctgaatgcggctaatcctaaccacggagcaagggtgtgtgagccaacacataccttgtcgtaatgcgtaagttcgtggcggaaccgactactttgggtgtccgtgtttccttttattcttgtattggctgcttatggtgacaatcatagattgttatcataaagcgacttggattggccatccagtggaagtgaaataccttgtctacctgtctgttggttttactccatttgactaattcacttacaagcttattgctataatttttattgactaaacaacaatactaca

SEQ ID NO: 228 - DNA, 581 bp, synthetic construct
  source 1..581: mol_type = other DNA
cgtaggtccacatattgttgctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 229 - DNA, 773 bp, synthetic construct
  source 1..773: mol_type = other DNA
accaccgtgaatgcgacaatggcgcgcctcggcaaggccgccgataccgcactgtgctacttcggtaccactctgcgggaatgacaatatgcccggtgtatagtttgcctgtttctcgcaccgttaccgctcgttcgggaatgtgaaactggcacccctcctctcccctaccaccctttctccttcgcccccattcataatttacaacgccgcacacagcggcggccgccaagggctagcctggcggttataaaaggaacctgggtctttccctcttcaagccaaaaggtaggttccctgtgtccctgaatgctcggtgaggaatgctgcaccgtaacgctttgtgaagtgtttgcaagttctggcccggcaagcctacagagtgctgtgatccgctgcggacgccatcctggtaacaggacccccagtgtgcgcaacagtatgttcagacttcggtttgttcacttgctttcatggaccattgcgcgaaagtgcgtgcgccatatccctgtacttcaggtgtgcttctctggaccctaggaatgctgcgaaggtaccccgtttcggcgggatctgatcgcaggctaattgtctatgggttcagtttcctttttctttactccacaattgactgcttaactgactctggatcttgtgcttccactgctctttctgctctcaaaacggcttcacttaccaactctcacctttcgaccaacaccatttacacactaacttttttcgactcttctgactcctggcttggtgaagac

SEQ ID NO: 230 - DNA, 623 bp, synthetic construct
  source 1..623: mol_type = other DNA
tgagggagatactaaccctgccaccctttctccttcgcccccattcataatttacaacgccgcacacagcggcggccgccaagggctagcctggcggttataaaaggaacctgggtctttccctcttcaagccaaaaggtaggttccctgtgtccctgaatgctcggtgaggaatgctgcaccgtaacgctttgtgaagtgtttgcaagttctggcccggcaagcctacagagtgctgtgatccgctgcggacgccatcctggtaacaggacccccagtgtgcgcaacagtatgttcagacttcggtttgttcacttgctttcatggaccattgcgcgaaagtgcgtgcgccatatccctgtacttcaggtgtgcttctctggaccctaggaatgctgcgaaggtaccccgtttcggcgggatctgatcgcaggctaattgtctatgggttcagtttcctttttctttactccacaattgactgcttaactgactctggatcttgtgcttccactgctctttctgctctcaaaacggcttcacttaccaactctcacctttcgaccaacaccatttacacactaacttttttcgactcttctgactcctggcttggtgaagac

SEQ ID NO: 231 - DNA, 748 bp, synthetic construct
  source 1..748: mol_type = other DNA
atcgaaaggcttatacgatcccacgtgtgtatgccaacctacatggactcgtgcatgtaggttggattcacaaccctttcataaccccccccttttaacccaacccttcgtaaccgtacgcttcactcgcctttgggtatagcggcccaatgtgctgaagaaaaggatacgctataaggggccaacgggtggtggcccttaagaccacccaacctagaagcttgtacactcgggcaatagtgaggcccacatccagtgggtcaagcccaaagcattcttgttccccggtatgatctcataagctgtacccacggctgaaagagtgattatcgttatcccactcagtacttcggagagcctagtacaccacttggaaatggaagtctgtgatccggggttgaccctgaaccccagaaactcatgatgaggctaaccttcccgaacacggcgacgtgtggttagcctgcgctggcatgaggcctctttgtaggcagactgaaatggaagggtgacgaagagccgactgagctactgttttattcctccggccccctgaatgcggctaatcctaactcctggtccagtacttgtaacccaacaggtggctggtcgtaatgcgtaagccgggagcggaaccgactactttggggcgtccgtgtttctcaatattattcatttctagcttatggtgacaatttatgattgcagagattgtgctgtatttgtgtctgagagaagaagtaacaat

SEQ ID NO: 232 - DNA, 768 bp, synthetic construct
  source 1..768: mol_type = other DNA
tcggtgtcaggcctcaccttttaaaacagcctgtgggttgttcccacccacagggcccactgggcgctagcactctgatttcacgaaatctttgtgcgcctgttttatatcccttcccccagtctgaaacatagaagcaatgcgcatcactgatcaatagcaggcgtaacgcgccagttacgtcatgatcaagcacatctgtctccccggaccgagtatcaatagactgcttgcgcggttgaaggagaaacagttcgttacccggctaactacttcgagaagcccagtagcaccatggaagctgcagggtgtttcgctcagcacttcccccgtgtagatcaggtcgatgagtcactgcaacccccacaggcgactgtggcagtggctgcgttggcggcctgcctatggggagacccataggacgctctaatgtggacatggtgtgaagagcctattgagctagttagtagtcctccggcccctgaatgcggctaatcctaactgcggagcacacgccctcaacccagagggtagtgtgtcgtaacgggcaactctgcagcggaaccgactactttgggtgtccgtgtttcttattattcttatattggcagcttatggtgacaattacagaattgttaccatatagctattggattggccatccggtgtgcaatagagctgttatatacctgtttgttggttttgtgccactaactttaaaatctatagtcactcttaatttcatattggctctcaacacaattaaac

SEQ ID NO: 233 - DNA, 669 bp, synthetic construct
  source 1..669: mol_type = other DNA
cgtgaatctcggcatcccatccatgcaacttagaagttttttaaacaaagttcaatagaagggggtacaaaccagtgccaccacgaacaagcacttctgtttccccggtgaagctgcatagactgtccccacggttgaaagcggcagatccgtcatccgctttggtacttcgagaaacctagtatcaccttggaatcttcgatgcgttgcgctcagcactcaaccccagagtgtagcttaggttgatgagtctggacaatcctcactggcgacagtggtccaggctgcgttggcggcctacctgtggtgaaaaccacaggacgctagatgtgaacaaggtgtgaagagtctattgagctaccaaagagtcctccggcccctgaatgcggctaatcctaaccacggagcaagtacccacaaaccagtgggcagcttgtcgtaacgcgcaagtctgtggcggaaccgactactttgggtgtccgtgtttcctttttattttatcatggctgcttatggtgacaatctaagattgttatcatatagctattggattggccatccggtgactaacagagatattatatacctatttgttggttttgttagattaaatactatcacattcaaaactcttctttatatcatacaattgaacagtagaaagagaaa

SEQ ID NO: 234 - DNA, 762 bp, synthetic construct
  source 1..762: mol_type = other DNA
cgatctttgtcaaataccgtttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcata

SEQ ID NO: 235 - DNA, 748 bp, synthetic construct
  source 1..748: mol_type = other DNA
tttaaaacagctctagggttgttcccaccctagaggcccaagtggcggctagcactctggtattacggtacctttgtgcgcctgttttatatcccttcccccatgtaacttagaagatattaaacaaagttcaataggagggggtacaaaccagtgccaccacgaacaaacacttctgtttccccggtgaagctacatagactgttcccacggttgaaagtggcagatccgttatccgctttggtacttcgagaaacctagtaccaccttggaatcttcgatgcgttgcgctcagcactcaaccccagagtgtagcttaggtcgatgagtctggacgatcctcactggcgacagtggtccaggctgcgttggcggcctacctgtggcgaaagccacaggacgctagttgtgaacaaggtgtgaagagtctattgagctaccaaagagtcctccggcccctgaatgcggctaatcccaaccacggagcaagtgcccacaaaccagtgggtggcttgtcgtaatgcgtaagtctgtggcggaaccgactactttgggtgtccgtgtttccttttatttttaaactggctgcttatggtgacaatctaagattgttatcatatagctattggattggccatccggtgactaacagagatcttgcatacctgtttgttggttttactaaactagatatagttacatttaaaactcttctttatatcatacagttgaatagtagaaagagaaa

SEQ ID NO: 236 - DNA, 675 bp, synthetic construct
  source 1..675: mol_type = other DNA
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttcctgtgcgtcctgtgagagatttccaacaattgaacagactagaaccacttgttttatcaaaccctcacagaataagataaca

SEQ ID NO: 237 - DNA, 675 bp, synthetic construct
  source 1..675: mol_type = other DNA
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttatttttaaactggctttttatggtgacaactcctggtagacgttcctgtgcgtcctgtgagagatttccaacaattgaacagactagaaccacttgttttatcaaaccctcacagaataagataaca

SEQ ID NO: 238 - DNA, 748 bp, synthetic construct
  source 1..748: mol_type = other DNA
tttaaaacagctctagggttgttcccaccctagaggcccaagtggcggctagcactctggtattacggtacctttgtgcgcctgttttatatcccttcccccatgtaacttagaagatattaaacaaagttcaataggagggggtacaaaccagtgccaccacgaacaaacacttctgtttccccggtgaagctacatagactgttcccacggttgaaagtggcagatccgttatccgctttggtacttcgagaaacctagtaccaccttggaatcttcgatgcgttgcgctcagcactcaaccccagagtgtagcttaggtcgatgagtctggacattccccactggcgacagtggtccaggctgcgttggcggcctacctgtggcgaaagccacaggacgctagttgtgaacaaggtgtgaagagtctattgagctaccaaagagtcctccggcccctgaatgcggctaatcccaaccacggagcaagtgcccacaaaccagtgggtggcttgtcgtaatgcgtaagtctgtggcggaaccgactactttgggtgtccgtgtttccttttatttttaaactggctgcttatggtgacaatctaagattgttatcatatagctattggattggccatccggtgactaacagagatcttgcatacctgtttgttggttttactaaactagatatagttacatttaaaactcttctttatatcatacagttgaatagtagaaagagaaa

SEQ ID NO: 239 - DNA, 23 bp, synthetic construct
  source 1..23: mol_type = other DNA
caaacaccattgtcacactccaa

SEQ ID NO: 240 - DNA, 767 bp, synthetic construct
  source 1..767: mol_type = other DNA
gcgcaccctgcaaattaaatttaaaacagctctgggggtgttcccaccctagaggcccacgtggcggctagcactctggtatcacggtacctttgtgcgcctgttttatatccctttccccatgcaacttagaagtttttaaacaaagttcaatagaagggggtacaaaccagtgccaccacgaacaagcacttctgtttccccggtgaagctgcatagactgtccccacggttgaaagcggcagatccgttatccgctttggtacttcgagaaacctagtatcaccttggaatcttcgatgcgttgcgctcagcactcaaccccagagtgtagcttaggttgatgagtctggacgatcctcactggcgacagtggtccaggctgcgttggcggcctacctgtggtgaaaaccacaggacgctagatgtgaacaaggtgtgaagagtctattgagctaccaaagagtcctccggcccctgaatgcggctaatcctaaccacggagcaagtacccacaaaccagtgggcagcttgtcgtaacgcgcaagtctgtggcggaaccgactactttgggtgtccgtgtttcctttttattttatcatggctgcttatggtgacaatctaagattgttatcatatagctattggattggccatccggtgactaacagagatattatatacctatttgttggttttattagattaaatactatcacattcaaaactcttctttatatcatacaattgaacagtagaaagagaaa

SEQ ID NO: 241 - DNA, 654 bp, synthetic construct
  source 1..654: mol_type = other DNA
tttgctcagcgtaacttctccgggttacgtggagaccaaaaggctacggagactcgggctacggccctggagcacctaggtgctcctaaagacgttagaagttgtacaaactcgcccaatggggccccccaaccaggggggtagcgggcaggcacttctgtttccccggtatgatctcataggctgtacccacggctgaaagagagattatcgttacccgcctcactacttcgagaagcccagtaatggttcatgaagttgatctcgttgacccgtgtcgcttcccccgcgacacagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttatttttaaactggctttttatggtgacaactcctggtagacgttcctgtgcgtcctgtgagagatttccaacaattgaacagactagaaccacgccgccacc

SEQ ID NO: 242 - DNA, 1494 bp, synthetic construct
  source 1..1494: mol_type = other DNA
atggctctccccgtgaccgctctgctgctccctctggccctccttctgcacgcagccagaccacaggtcaagctggaggagtctggtggcggtctggtgcaggcagggaggagcctgaggctgagctgtgcagcttccgagcacacattctcaagccacgtcatggggtggttcagacaggctcccggtaaagagagggagtccgtcgccgtgatcggatggcgggacatctccacctcctacgccgactctgtgaagggccggttcacaatctcacgcgataatgccaagaagacactgtatctgcagatgaattccttgaagcccgaagacaccgccgtctattactgtgctgctagacggatcgacgctgccgacttcgacagctggggacagggtacccaagtgaccgtttcctccggaggcggaggttctggaggaggtgggtcaggtggaggtggctccgaggtgcagctggtcgagtctggcggtggcttggtccaggctggaggcagtctcagactctcctgcgctgcttcagggcggaccttcaccatgggctggttcaggcaggccccaggtaaggagagggagttcgtggccgccatctccctctcccctaccctggcatactacgctgagtccgtgaagggacggtttaccatctcccgggataacgcaaagaacactgtggtcctccaaatgaactccctcaaacccgaggacaccgctctctactattgtgccgcagatcggaagagcgtcatgtccatccggcccgattactggggccaaggcacacaggtgactgtgtccagcacctccaccaccaccccagcaccaaggcctccaacccctgcaccaaccatcgcctcccagccactgtctttgcggccagaagcatgccgcccagcagcaggtggagccgtgcatacaagaggcctggacttcgcctgcgatatctacatctgggctcctctggccggaacatgcggagtcctgctcttgtccctggtgatcaccctgtactgcaagcggggtcggaagaagctcctctacatcttcaagcagcccttcatgagacccgtccagaccacccaggaggaggacgggtgctcatgcaggttccccgaagaggaggagggtggctgtgagctgcgggtgaagttcagcaggtcagcagacgcccctgcctatcagcagggccaaaaccagttgtacaacgagctgaatctggggagacgggaggagtacgatgtccttgacaagagaaggggccgggatccagagatgggcgggaagccaagacggaagaatcctcaggagggtctgtataacgagctgcagaaggacaagatggccgaggcctactccgagatcggcatgaaaggggagcgccgcagaggaaaaggtcacgatggtctgtaccaggggttgagcaccgctaccaaggatacttacgacgctctgcacatgcaagctctgccaccccgg

SEQ ID NO: 243 - DNA, 1479 bp, synthetic construct
  source 1..1479: mol_type = other DNA
atggcactcccggtaaccgccttattgcttccccttgccctcttgctccacgcagcacgccccgatatagtcttgactcaatccccacccagtttggcaatgtcattaggcaaacgagcaacaatttcatgtagggcatccgaaagtgtaacgattttggggagtcatttaattcattggtaccaacaaaagcctggacaacccccgacgctcttgatccaattagcatctaacgtccaaaccggagtccccgcacgattctcaggatccggttcccggactgattttacattaactattgatccggtagaggaagatgacgtcgctgtctattattgtcttcaaagtaggacgattccacggacattcggtggcggaactaaattggagattaaaggttccacctctggtagtgggaaacccgggtccggtgaagggtccactaaaggccaaattcaactcgttcaatccggaccagaactgaagaagccaggagaaactgtcaaaataagctgtaaagcttccggttatacatttacagattattccataaattgggtgaaaagggcgccaggaaaagggttaaagtggatgggttggattaatacagagactcgggaacctgcatatgcttatgattttaggggaaggtttgccttttctctggagacttccgcttcaactgcttatctccaaattaataatcttaaatatgaggacacagcaacatacttctgtgctttggactatagttatgctatggattactggggacaaggaaccagtgtcactgtaagttccgctgctgcgacgaccactcctgcaccgcgaccacccactcctgcccctactattgctagtcaaccacttagcttgcgacctgaggcatgtcggcccgcggcaggtggcgcagtccacaccaggggtttagactttgcttgtgatatttatatttgggcaccactcgccgggacttgcggtgttcttctcttgtcccttgttataactctttattgtaagcgcggaaggaagaaattgttatatattttcaaacaaccttttatgcgacccgtacaaacaactcaggaagaggacgggtgttcttgtcggtttccagaagaggaagagggtgggtgtgaactccgggtcaaatttagtaggtcagcagatgcgccggcgtaccaacaaggccaaaaccaactgtataatgaactcaatctcggtaggcgtgaggaatatgatgtccttgataaaaggcgcgggagagatccagaaatgggcggaaaaccacggcgaaagaatccgcaggaagggttatataacgaacttcaaaaggataaaatggctgaagcttattccgaaattggcatgaaaggagagcgacgtaggggcaaagggcatgatggcctttaccaagggctctcaaccgctacaaaagatacttacgacgctttacatatgcaagcacttccacccagg

SEQ ID NO: 244 - DNA, 1446 bp, synthetic construct
  source 1..1446: mol_type = other DNA
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 245 - DNA, 1476 bp, synthetic construct
  source 1..1476: mol_type = other DNA
atggccctccccgtcacagctctcctgctcccactggcccttcttttgcacgctgctcgccccgatatcgtgctcacccagtcacctccaagccttgccatgagcctcgggaaacgggctaccatctcctgccgggcttcagagtccgtcaccatcctcgggtcacacctcatccactggtaccaacagaaaccagggcagcctcctaccctcttgatccagttggcctccaacgtgcaaactggggttcccgccaggttcagtggctccggatcccggacagatttcacacttaccatcgatcctgtggaggaggacgatgtggccgtctattactgcctgcagtctcgcaccatccctcggaccttcggtggaggcaccaagctcgagatcaagggtagcacctccggctctggaaagccaggctctggtgagggttctaccaagggccaaatccagctggtccagtctgggcccgagctgaagaaacccggggagaccgtgaagatctcctgcaaggcctccggttataccttcaccgactactccatcaactgggtcaagcgcgctcctggaaagggcctcaagtggatgggctggatcaacaccgaaacccgcgagcctgcctatgcttacgacttcaggggccggttcgctttctcactggagacctccgcttccacagcctacctccagatcaacaacctcaagtacgaagacaccgccacctatttctgcgctctcgactattcctacgctatggactactggggtcagggcacctctgtgaccgtctctagcgcagccgccaccacaacaccagccccacggccacctactcccgcacccaccatcgcatcccaaccactcagtctgaggcccgaggcctgtagacctgctgctggaggcgcagtgcatacccgcggtctcgacttcgcctgcgacatctatatctgggccccattggcaggtacctgtggcgtgctgctgctgtcactcgtcatcaccctgtactgccggagtaagcgctctaggctgttgcacagcgactacatgaacatgaccccaagaagaccagggcctacccggaagcactaccagccatacgcacctccccgggactttgccgcctatcggtctcgggtgaagttctcacgctccgctgatgccccagcataccagcaggggcagaaccagctgtacaatgagctcaacctcggtcgccgcgaagagtacgacgtgctcgacaagagaaggggcagggaccctgagatgggaggcaagccccgcagaaagaatccccaggaaggtctgtacaacgagctgcaaaaggataagatggctgaggcctacagcgagatcggcatgaagggcgaaaggagacggggaaagggccacgacgggctctaccagggactctccaccgccaccaaggacacctacgacgccctccacatgcaggctctgccacccagg

SEQ ID NO: 246 - DNA, 1458 bp, synthetic construct
  source 1..1458: mol_type = other DNA
atggcacttcccgtcaccgctctcctgctgcccctcgcactgctgctccatgcagcccgcccagacatcgtcctgacccagtcccctccctccctcgcaatgtccctcgggaaacgggccaccatcagctgccgggcctctgagtcagtgacaatcctcggaagccatctgatccattggtaccagcagaaacccggtcagcctccaaccctcctcatccagctggcctccaacgtgcagacaggagtccccgctcggttctcaggcagcggttccaggaccgacttcaccctgaccatcgaccccgtggaagaggacgatgtggctgtgtactactgcctccagtcccggaccatcccacggaccttcggaggtgggacaaagctggagatcaaaggcagcaccagcggttctggcaagccagggtcaggtgaggggagcacaaagggtcagatccagctggtgcagagcggtcccgagctgaagaagcccggggagaccgttaagatctcctgcaaggctagcgggtacaccttcaccgactatagtatcaactgggtcaagcgcgctcctggcaaggggctcaagtggatggggtggatcaacaccgaaaccagggagcccgcatacgcttatgactttcggggccggttcgccttttccctggagaccagcgcctctaccgcctacctccagatcaacaacctgaagtacgaggacaccgccacctacttctgcgcactcgactactcctacgctatggactactggggtcagggtacctccgtcaccgtctccagcatcgaggtcatgtaccctcctccctacctggacaacgagaagtccaacggcaccatcatccatgtgaagggcaagcatctctgccccagcccactgttccccggaccctctaagcccttctgggtcctggtcgtcgtcggcggtgttctggcttgctacagcttgctggtcaccgtcgccttcatcatcttctgggtgcgctccaagaggagccggctgctgcatagcgactacatgaacatgacccctagaaggcctggtccaacccgcaagcactaccagccttacgcccctccacgggacttcgcagcctaccggtcacgggtgaagttctctcggagcgcagatgccccagcataccagcagggccagaaccagctgtacaacgaacttaaccttggtcggcgggaggaatacgatgtgctggacaagcgcaggggtcgggatcctgaaatgggcgggaaaccacgccggaagaacccacaggaggggctctataacgagctccaaaaggataagatggctgaggcttacagcgagattggaatgaagggagaaagaagacggggcaagggtcacgacgggttgtaccagggtctgagcaccgccaccaaggacacctacgacgccctccacatgcaagcccttccaccccgc

SEQ ID NO: 247 - DNA, 1491 bp, synthetic construct
  source 1..1491: mol_type = other DNA
atggcactccccgttaccgcccttctgctgcctctcgccttgctgctgcacgcagccagaccacaggtcaagctggaggagtctggtggcgggctcgttcaagcaggtcggagtctccgcctgtcttgcgcagcatcagagcataccttctcctcacacgtgatggggtggttcaggcaagctcccggtaaggagagggagtccgtggccgttatcggctggcgcgatatcagcacctcctacgcagacagcgttaagggccggttcactatctccagggacaacgctaagaagacactctacctccagatgaacagtctgaagcccgaggacaccgcagtgtactattgcgctgctcggcggatcgatgctgccgacttcgacagctggggtcaagggacccaggtcaccgtttccagcggaggtggcggaagtggtggcggaggatcaggtggtggaggctccgaggtccagctggtggaatcaggaggcggcttggtgcaggctggtgggtctttgcggttgtcctgcgcagcttccggcaggaccttcaccatgggatggttcagacaagccccaggtaaggagcgggagtttgtggccgcaatctcactgtctcccaccctcgcttactacgccgagagtgtgaaggggcgcttcacaatcagtcgcgacaacgcaaagaacaccgtcgtcctgcaaatgaactccctgaagcctgaggataccgcactctattactgcgccgccgatcggaagagcgtcatgtccatccggcccgactattggggccaaggcacccaagtgaccgtcagctccacctccacaaccactcccgccccaagaccacctaccccagccccaacaatcgcatcccagcctctgtcccttcggcccgaagcttgtcgccctgcagcaggtggagcagtgcacacccggggactggacttcgcctgcgacatctacatctgggcacccctggctggaacctgcggcgtgttgctgctgagcctggtgatcaccctctactgccgctctaagagaagccggctgctgcatagcgactacatgaacatgacccctaggagaccaggacccacccggaagcactaccagccttacgctcctccacgggatttcgctgcttaccgcagccgggtgaagttttccaggtcagctgacgcccctgcctaccagcagggccagaaccaattgtacaacgaactgaatctgggacggcgcgaggaatacgacgtcctggacaagaggcggggtagagatcccgagatgggcgggaaacctcggcggaagaaccctcaggaggggctctacaacgagctgcagaaggataagatggccgaagcctactccgagatcgggatgaagggtgaacggaggaggggcaagggacacgacggcctgtatcagggcctcagcaccgctaccaaggacacctacgacgccctgcacatgcaggctctcccaccacgg

SEQ ID NO: 248 - DNA, 1476 bp, synthetic construct
  source 1..1476: mol_type = other DNA
atggctcttcccgtcaccgctttgctgctgcccctggcactcctcctccatgctgctcggcctcaggtgaagctggaggagagtggtggcggtctggtgcaagctggcagatctctgcgcctgtcttgcgcagccagcgaacacaccttctcctcccacgtgatggggtggtttcggcaggcacccgggaaagagcgcgagtccgtcgcagtcatcgggtggcgggacatctctaccagctacgcagattccgtcaagggccggttcaccatttcccgggataacgctaagaagaccctctacctgcaaatgaactctctgaagcccgaagacaccgccgtctactattgcgcagcaaggcgcatcgacgctgccgacttcgactcttggggccaaggaacccaggtcaccgtgtcttccggaggaggaggctccggtggtggaggttctggaggtggcggctcagaggtgcagctcgtggagagcggtggtggactcgttcaggcaggcggcagtttgcggctgtcctgtgcagcctccggtcgcactttcactatgggatggttccgccaggctcctggtaaagaaagggagttcgtggccgccatcagtctgagccccaccctcgcatactacgccgagagcgtgaagggtaggttcactatcagccgggacaacgccaagaacaccgtggtgctccagatgaattccctgaagcctgaggataccgccctctactactgcgctgccgaccgcaagagcgtgatgagcatccggcctgactattggggtcaggggacacaggtgaccgtcagcagcatcgaggtgatgtatccaccaccctacctcgacaacgagaagtccaacggcaccatcatccacgtcaaggggaagcacctctgcccttcccctctgttccctggcccctcaaagcccttctgggtcctggtggtggttggtggggtgctggcttgctactccctgctcgtgaccgtggctttcatcatcttctgggttcggagcaaacggtccagactgctgcactccgactacatgaacatgaccccaagaagacctgggcccacacggaagcattaccaaccctatgcaccacctcgggatttcgccgcctacagatcccgggtcaagttctccaggtccgccgatgcaccagcctatcagcaggggcaaaaccagctgtataatgagctgaaccttggacggcgcgaggagtacgacgtgctcgacaaaagacgcggtcgcgacccagagatgggcggcaagcctagacgcaagaatccccaggaggggctctataacgagttgcagaaggataagatggccgaggcctacagcgagatcgggatgaaaggcgaaagacggcgcggaaagggtcacgacggactctaccagggcctgagcacagccaccaaagacacctacgacgctctgcatatgcaagcactgcctccccgg

SEQ ID NO: 249 - DNA, 1452 bp, synthetic construct
  source 1..1452: mol_type = other DNA
atggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 250 - DNA, 1464 bp, synthetic construct
  source 1..1464: mol_type = other DNA
atggctctgcctgtgacagctctgctgctgcctctggctctgcttctgcatgccgccagacctgacatccagatgactcagagccccagcagcctgtctgcctctgtgggagacagagtgacaattacctgccgggccagccaggatgtgaatactgctgtcgcctggtatcaacaaaagcctggcaaggcccctaagctcctgatctacagcgccagctttctgtacagcggcgtgcccagcagattctccggaagcagaagcggcacagatttcacactgaccataagcagcctgcagccagaggatttcgccacctactattgccagcagcactacaccacacctccaacctttggccagggcaccaaggtcgagattaagagaacaggcagcacatctggctctggcaaacctggatctggcgagggctctgaagtccagctggtggaatctggcggaggactggttcaacctggcggctctctgagactgtcttgtgccgcctccggcttcaacatcaaggacacctacatccactgggtccgacaagccccaggcaaaggacttgagtgggtcgccaggatctaccccaccaacggctacaccagatacgccgactctgtgaagggcagattcaccatctctgccgacaccagcaagaataccgcctacctgcagatgaactccctgagagccgaagataccgctgtgtattactgttccagatggggaggcgacggcttctacgccatggatgtttggggccaaggcaccctcgtgaccgtttcttctaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggctcctctggccggaacatgtggcgttctgctgctgagcctggtcatcaccctgtactgtaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 251 - DNA, 1446 bp, synthetic construct
  source 1..1446: mol_type = other DNA
atggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 252 - DNA, 1473 bp, synthetic construct
  source 1..1473: mol_type = other DNA
atgctgctgctggtcacatctctgctgctgtgcgagctgccccatcctgcctttctgctgatccctgagatcgtgctgacacagagccccgccacactgtcactttctccaggcgaaagagccacactgagctgtagcgccagctccagcatcagcagcaactacctgcactggtatcagcaaaagcccggacaggcccctcggttcctgatctacagaaccagcaatctggccagcggcatccccgccagattttctggctctggcagcggaaccgacttcaccctgaccataagcagcctggaacctgaggacttcgccgtgtactactgtcagcaggccagctctatccccagaatgttcacctttggccagggcaccaagctggaaatcaagggcagcacaagcggcagcggcaaacctggatctggcgagggttctaccaagggcgaagttcagctgcagcagtctggcgccgaagtgaagaaacctggcgcctctgtgaaggtgtcctgcaaggcctctggctacacctttaccaactactggatgcactgggtccgacagaggcctggacaaggactggaatggatgggcgagatcgaccccagcgacaactacgccaactacaatcaagagttccagggcagagtgaccatcaccgtggataagagcgccagcaccgcctacatggaactgagcagcctgagaagcgaggataccgctgtgtactattgcgccagacacgacggctactttggcgccctggattattggggccagggaaccacagtgaccgtgtctagcgccgctgccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctcgggatttcgccgcctacagatccagagtgaagttctccagaagcgccgacgctcctgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtatcagggactgagcaccgccaccaaggacacctatgatgccctgcacatgcaggccctgcctccaaga

SEQ ID NO: 253 - DNA, 1464 bp, synthetic construct
  source 1..1464: mol_type = other DNA
atggctctgcctgtgacagctctgctgctgcctctggctctgcttctgcatgccgccagacctgacatcgtgatgacacagtctcccgccacactgtctctgagccctggcgaaagagccacactgagctgcagaagctccaagagcctgcagaacgtgaacggcaacacctacctgtactggttccagcagaagcccggccagtctcctcagctgctgatctacagaatgagcaacctgaacagcggcgtgcccgacagattttctggcagcggaagcggcaccgagttcaccctgaccataagcagcctggaacctgaggacttcgccgtgtactactgtatgcagcacctggaataccccatcaccttcggagccggcaccaagctggaaatcaaaggcggcggaggatctggcggaggtggaagtggcggaggcggatctgaagtgcagctggttgaatctggcggcggactggttaagcctggcggatctctgaagctgagctgtgccgccagcggctacacctttaccagctacgtgatgcactgggtccgacaggcccctggcaaaggacttgagtggatcggctacatcaacccctacaacgatggcaccaagtacaacgagaagttccagggcagagtgaccatcagctccgacaagagcatcagcaccgcctacatggaactgagcagcctgagaagcgaggataccgccatgtactattgcgccaggggcacctactactacggcaccagagtgttcgactactggggccagggaaccctggtcaccgtgtctagcatcgaagtgatgtaccctccaccttacctggacaatgagaagtccaacggcaccatcatccacgtgaagggcaaacatctgtgcccctctccactgttccccggacctagcaagcctttctgggtgctcgttgttgttggcggcgtgctggcctgttactctctgctggttaccgtggccttcatcatcttttgggtccgaagcaagcggagcagactgctgcacagcgactacatgaacatgacccctagacggcccggaccaaccagaaagcactaccagccttacgctcctcctcgggatttcgccgcctacagatccagagtgaagttctccagaagcgccgacgctcctgcctatcagcagggacagaaccagctgtacaatgagctgaacctggggcgcagagaagagtacgacgtgctggataagcggagaggcagagatcctgagatgggcggcaagcccagacggaagaatcctcaagagggcctgtataacgaactgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 254 - DNA, 1443 bp, synthetic construct
  source 1..1443: mol_type = other DNA
atggctctgcctgtgacagctctgctgctgcctctggctctgcttctgcatgccgctagacctcaagtgaccctgagagagtctggccccgctctggttaagcccacacagaccctgacactgacctgcaccttcagcggctttagcctgagcacatctggcatgggcgtcggctggattagacagcctcctggaaaagccctggaatggctggcccacatttggtgggacgacgacaagagatacaaccccgcactgaagtcccggctgaccatcagcaaggacaccagcaagaaccaggtgttcctgaccatgaccaacatggaccccgtggacaccgccacctactactgcgccagaatggaactgtggtcctactacttcgactactggggccagggcaccaccgtgacagtttctagcggaggcggaggatctggtggcggaggaagtggcggaggcggttctgaaaatgtgctgacacagagccccgccacactgtctgttacacctggcgagaaggccaccatcacctgtagagccagccagagcgtgtcctacatgcactggtatcagcagaagcccggacaggcccctagactgctgatctacgacgccagcaatagagcctctggcgtgcccagcagattttctggcagcggctctggcaccgatcacaccctgacaatctctagcctggaagccgaagatgccgctacgtactactgcttccaaggcagcgtgtaccccttcacctttggccagggaacaaagctggaaatcaagatcgaagtgatgtacccgcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgtctaccgccaccaaggatacctatgacgccctgcacatgcaggccctgcctccaaga

SEQ ID NO: 255 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgccagcagcagcgtgtcctacatgcactggtatcagcagaagcccggacaggcccctagactgctgatctacgataccagcaagctggcctctggcatccccgccagattttctggaagcggcagcggcaccgatttcaccctgaccataagcagcctggaacctgaggacgtggccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacatctggcatgggagttggctggatcagacagcaccctggcaaaggcctggaatggatcggccacatttggtgggacgacgacaagcggtacaaccccgctctgaagtccagagtgaccatcagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccgcagcagagtgaagttcagcagatccgccgatgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 256 - DNA, 1449 bp, synthetic construct
  source 1..1449: mol_type = other DNA
atggctctgcctgtgacagctctgctgctgcctctggctctgcttctgcatgccgctagacctgaagtgcagctggttcagtctggcgccgaagtgaagaagcctggcgagagcctgaagatcagctgcaaaggcagcggctacagcttcagcagctcttggatcggctgggtccgacagatgcctggcaaaggccttgagtggatgggcatcatctaccccgacgacagcgacaccagatacagccctagcttccagggccaagtgaccatcagcgccgacaagagcatcagaaccgcctacctgcagtggtccagcctgaaggcctctgacaccgccatgtactactgcgccagacacgtgaccatgatctggggcgtgatcatcgatttctggggccagggcacactggtcacagtttctagcggaggcggaggatctggtggcggaggaagtggcggaggcggatctgctattcagctgacacagagccctagcagcctgtctgcctctgtgggcgacagagtgaccattacctgtagagccagccagggcatctctagcgccctggcttggtatcagcagaagccaggcaaggcccctaagctgctgatctacgatgccagcagcctggaaagcggcgtgccaagcagattttctggcagcggcagcggaaccgacttcaccctgaccatatctagcctgcagcctgaggacttcgccacctactactgccagcagttcaacagctacccctacaccttcggccaggggaccaagctggaaatcaagatcgaagtgatgtacccgcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttctccagatccgctgatgcccctgcctatcagcagggccagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggacacatatgatgccctgcacatgcaggccctgcctccaaga

SEQ ID NO: 257 - DNA, 1449 bp, synthetic construct
  source 1..1449: mol_type = other DNA
atggctctgcctgtgacagctctgctgctgcctctggctctgcttctgcatgctgctagacctcaggtgcagctggttcagtctggcgccgaagtgaagaaacctggcagcagcgtgaaggtgtcctgcaaggcctctggccacaccatcagcagctacgcctacagctgggttcgacaggctcctggacaaggcctggaatggatgggcgatatcatccctgcctacggcagccccaattacgcccagaaattccagggcagagtgaccatcaccgccgacgagtctacaagcaccgcctacatggaactgagcagcctgagaagcgaggacaccgccgtgtactactgcgccagagaggacttcggcaagaactacgccatggacgtgtggggccagggcacactggttacagtttctagcggaggcggaggatctggtggcggaggaagtggcggaggcggttctgaaattgtgctgacacagagccccggcacactgtcactttctccaggcgaaagagccacactgagctgtagagcctctcagcacgtgtccagccactacctggcttggtatcagcagaagcctggacaggcccctcggctgttgatctatggcgcctcttccagagccacaggcatccccgatagattcagcggctctggcagcggcaccgatttcaccctgacaatcagcagactggaacccgaggactttgccgtgtattattgccagcactacggccagagccagttcacctttggacagggcaccaaggtggaaatcaagatcgaagtgatgtacccgcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggtcaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgccgatgctcccgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 258 - DNA, 1449 bp, synthetic construct
  source 1..1449: mol_type = other DNA
atggctctgcctgtgacagctctgctgctgcctctggctctgcttctgcatgccgccagacctgagatcgtgctgactcagtctcccggcacactgtcactgtctccaggcgaaagagccacactgagctgtagagcctctcagcacgtgtccagccactacctggcttggtatcagcagaagcctggacaggcccctcggctgttgatctatggcgcctcttccagagccacaggcatccccgatagattcagcggctctggcagcggcaccgatttcaccctgacaatcagcagactggaacccgaggacttcgccgtgtactactgtcagcactacggccagagccagttcacctttggccagggcaccaaggtggaaatcaaaggcggcggaggatctggcggaggtggaagtggcggaggcggatctcaagttcagctggttcagtctggcgccgaagtgaagaaacctggcagcagcgtgaaggtgtcctgcaaggcctctggccacaccatcagcagctacgcctacagctgggtccgacaggctcctggacaaggcttggagtggatgggcgatatcatccctgcctacggcagccccaattacgcccagaaattccagggcagagtgaccatcaccgccgacgagtctacaagcaccgcctacatggaactgagcagcctgagaagcgaggacacagccgtgtattattgcgccagagaggatttcggcaagaactacgccatggacgtgtggggccagggaacactggttaccgtgtcctccatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttacagtggccttcatcatcttttgggtccgaagcaagcggagccggctgctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 259 - DNA, 1464 bp, synthetic construct
  source 1..1464: mol_type = other DNA
atggctctgcctgtgacagctctgctgctgcctctggctctgcttctgcatgccgccagacctgacatcgtgatgacccagacacctctgagcctgagcgtgacacctggacagcctgccagcatcagctgcaagtctagccagagcctggaaaccaccaccggcaccacctacctgaactggtatctgcagaagcccggccagtctcctcagctgctgatctacagagccagcaagagattcagcggcgtgcccgatagattttccggcagcggctctggcaccgacttcaccctgaagatctccagagtggaagccgaggacgtgggcgtgtactactgtctgcagctgctggaagatccctacaccttcggctgtggcaccaagctggaaatcaaaggcggcggaggatctggcggaggtggaagtggcggaggcggatctcaagttcagctggttcagtctggcgccgaagtgaagaaacctggcgcctctgtgaaggtgtcctgcaaggccagcggctacgatttcaccgactacatcatgcactgggtccgacaggcccctggccaatgtcttgagtggatgggctacatcaacccctacaacgacggcagcaagtacaccgacaagttccaagagcgcgtgaccatgaccagcgacaccagcatctctaccgcctacatggaactgagccggctgagatccgatgacaccgccgtgtactattgcgccagaggaacctactactacggccccgagctgttcgattattggggccagggaaccaccgtgaccgtgtcctctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagcagactgctgcacagcgactacatgaacatgacccctagacggcccggaccaaccagaaagcactaccagccttacgctcctcctagagacttcgccgcctaccggtccagagtgaagttcagcagatccgccgatgctcccgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 260 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgcctctagcagcgtgggctacatgcactggtatcagcagaagcctggacaggcccctcggctgctgatctacgatacaagcaagctggcctctggcatccccgccagattttctggaagcggcagcggcaccgatttcaccctgaccataagcagcctggaacctgaggacttcgccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacaagcacaatgggcgttggctggatcagacagcaccctggcaaaggcctggaatggatcggcttcatttggtgggacgacgacaagcggtacaaccccaacctgaagtccagagtgaccatgagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctcgggatttcgccgcctaccggtccagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 261 - DNA, 1443 bp, synthetic construct
  source 1..1443: mol_type = other DNA
atggcactgcccgttaccgccctcctgctgccactggccctgttgctccacgcagccaggcccgagattgtcctcacccaatccccagccaccctctcactgtcacctggggagagggcaacactgtcctgcagcgcaagctcctccgtgggctacatgcactggtaccagcagaagccagggcaggctccaagactcctgatctacgacaccagcaagcttgcctccggtatccccgctcggttcagtggatccgggtccgggacagacttcactctcaccatctcctcactggaacctgaagatttcgccgtgtattactgcttccagggctcagtgtaccctttcaccttcggtcagggcacaaagctggaaatcaagggtggaggtggtagtggtggtggtggatcaggaggcggcggttcccaagtccagttgcaggagtccggtccaggcctcgtgaagccctcccagaccctgtctctgacttgcaccgtcagcggcggatctatcagtacctccaccatgggcgtgggctggattcggcaacaccccggcaagggactggagtggatcggtttcatctggtgggacgatgataagcggtataaccctaacctcaagtcccgggtcaccatgagcgtcgacacctcaaagaatcagttctccctgaagctctccagtgtcaccgcagccgatacagccgtgtactactgcgctcggatggagctctggtcctactacttcgactactggggacaaggcaccctggtcactgtcagctccatcgaggtcatgtaccctccaccatacctggacaacgagaagtctaacgggacaatcatccatgtgaagggcaagcacctttgccccagccccttgttcccaggcccctccaaacccttctgggtgctcgttgtggtgggtggggtgcttgcctgctactccctcctcgtcaccgtggccttcatcatcttctgggttcggagcaaacgcagccggctcctgcattccgactacatgaacatgactccaagacggccaggtcctaccaggaagcactatcagccctacgctccaccaagggacttcgccgcctacagatcccgggtgaagttctcccggtccgcagatgctcccgcttaccaacagggacagaaccaactgtataacgaactcaacctgggtcggcgggaagagtacgacgtgctggacaagcggcgcggccgggatcctgagatgggcggtaagccacgcaggaagaaccctcaggaggggttgtataacgagctgcagaaggataagatggccgaggcctacagcgagatcggcatgaaaggagaacgcaggagaggtaaggggcacgatggcctctaccaggggcttagcaccgccacaaaggacacctatgacgctttgcacatgcaggccttgccaccccgc

SEQ ID NO: 262 - DNA, 1443 bp, synthetic construct
  source 1..1443: mol_type = other DNA
atggcactccccgttaccgccctcctccttcccctggctttgcttctccacgctgccagacccgagatcgtgctcacccagtctccagccaccttgtccctcagccccggtgaacgcgcaactctgtcctgcagcgcttccagctccgtcggctacatgcactggtaccagcagaagccaggacaggctcccaggctcctcatctacgacaccagcaagctggccagtggtattcccgcccgcttctctggtagcggctccggcacagatttcaccctgaccatcagcagtctcgagccagaggacttcgcagtgtattactgcttccaggggtccgtgtaccccttcaccttcggtcagggcaccaagctggagatcaagggaggcggaggtagtggtggaggtgggagcggaggtggtggatcccaggtccaacttcaggagagcggtcctggccttgtgaagcccagccagaccctcagcctcacctgcaccgtgtccggtggttccatctccaccagcacaatgggcgtcgggtggatcaggcagcacccaggtaagggcctcgagtggatcggattcatctggtgggacgacgacaagaggtacaaccctaacctcaagtccagggtcaccatgtctgtcgataccagcaagaaccagttctccctcaagctgtctagcgtgacagctgccgacactgccgtctactactgtgcccggatggagctctggagctactacttcgactactggggccagggtacactggtgaccgtttcctccatcgaggtgatgtatcctcctccatacctcgacaacgagaagagcaacggcaccatcatccacgtcaaggggaagcacctctgcccctcacccctctttcccggtccctctaagcccttctgggtcctcgtggtcgtgggtggggtcttggcctgttactccctcctcgtcaccgtcgctttcatcatcttctgggtgcggagcaagagatcccggctgctccactccgattacatgaacatgactcctcggagaccaggccctacccgcaagcactatcagccctatgccccacccagggactttgctgcctacaggagccgggtgaagttcagccggagcgctgatgctccagcctaccagcaggggcagaaccagctctacaacgagctgaacctcggcaggcgggaagagtacgacgtgctggacaagcgccggggtagggacccagagatgggtggcaagccaaggcggaagaacccacaggagggactgtacaatgagctgcagaaagacaagatggccgaagcttactctgagatcggtatgaaaggcgaacgcaggcggggcaaaggccacgacgggctctaccagggcctgagcacagctaccaaggacacctacgacgctctgcatatgcaagccttgccaccccgc

SEQ ID NO: 263 - DNA, 1443 bp, synthetic construct
  source 1..1443: mol_type = other DNA
atggccctccccgtgacagctctcctcctgcccctggctctgctcctgcatgctgcacggcccgagatcgtcctgacccagagcccagcaaccctctccctctcccctggggagagagccaccctgagctgcagtgcctctagcagcgtggggtacatgcactggtatcaacagaagcctggtcaggcacccagactgctgatctacgacacaagcaagctggcttccggcatccctgctcggttctcaggctcagggagcggtaccgacttcaccctgaccatctccagccttgaacccgaggacttcgccgtctactactgcttccagggctccgtgtatccattcacattcggccagggcaccaagctcgagatcaagggtggaggaggcagtggtggtggtggttctggcggcggtggttcacaggtgcaactgcaggagagcggaccagggctggtcaaaccctcccagaccctgtccctcacctgcaccgtgtcaggtgggagcatcagcacctctaccatgggagtcgggtggatccggcaacaccccggaaagggcctggagtggattgggttcatctggtgggacgacgacaagcggtacaatcctaacctgaagtctcgggtcaccatgtccgtggacacctcaaagaaccagttctccctcaagctctcctctgtgaccgctgctgacacagccgtgtattattgcgcacgcatggagctgtggtcctactacttcgactattggggtcaaggcaccctcgtcacagtctcaagcatcgaggtgatgtatcctccaccttacctggacaatgagaagtcaaacggcacaatcatccatgtcaagggcaagcacctctgcccctcaccactcttcccaggcccctccaaaccattctgggtcttggtcgtcgtgggtggtgtcctcgcatgctactccctgctcgtcaccgtggccttcatcatcttttgggtgcggtccaagcggtcacggctcctgcacagcgactacatgaacatgacacctagacggccagggcctactcgcaaacactaccagccctacgcacctcctcgcgacttcgctgcctatcggagccgggtgaagttcagcagaagtgctgacgccccagcataccagcaggggcaaaaccagctgtacaacgagctcaacctcggacggcgggaagagtacgacgtgttggacaagcgcaggggtagggacccagagatgggtggcaagcctcgcagaaagaatccccaggaggggctgtacaatgagctgcagaaggataagatggcagaggcctacagcgagatcgggatgaagggagaacgcagacggggtaaggggcacgacggcctgtaccagggtctcagtaccgccaccaaggacacttacgatgctctccacatgcaagccctgccaccacgc

SEQ ID NO: 264 - DNA, 1464 bp, synthetic construct
  source 1..1464: mol_type = other DNA
atggctctgcctgtgacagctctgctgctgcctctggctctgcttctgcatgccgccagacctgacatcgtgatgacacagtctcccgccacactgtctctgagccctggcgaaagagccacactgagctgcagaagctccaagagcctgcagaacgtgaacggcaacacctacctgtactggttccagcagaagcccggccagtctcctcagctgctgatctacagaatgagcaacctgaacagcggcgtgcccgacagattttctggcagcggaagcggcaccgagttcaccctgaccataagcagcctggaacctgaggacttcgccgtgtactactgtatgcagcacctggaataccccatcaccttcggagccggcaccaagctggaaatcaaaggcggcggaggatctggcggaggtggaagtggcggaggcggatctgaagtgcagctggttgaatctggcggcggactggttaagcctggcggatctctgaagctgagctgtgccgccagcggctacacctttaccagctacgtgatgcactgggtccgacaggcccctggcaaaggacttgagtggatcggctacatcaacccctacaacgatggcaccaagtacaacgagaagttccagggcagagtgaccatcagctccgacaagagcatcagcaccgcctacatggaactgagcagcctgagaagcgaggataccgccatgtactattgcgccaggggcacctactactacggcaccagagtgttcgactactggggccagggaaccctggtcaccgtgtctagcatcgaagtgatgtaccctccaccttacctggacaatgagaagtccaacggcaccatcatccacgtgaagggcaaacatctgtgcccctctccactgttccccggacctagcaagcctttctgggtgctcgttgttgttggcggcgtgctggcctgttactctctgctggttaccgtggccttcatcatcttttgggtccgaagcagacggagcagactgctgcacagcgactacatgaacatgacccctagacggcccggaccaaccagaagacactaccagccttacgctcctcctcgggatttcgccgcctacagatccagagtgagattctccagaagcgccgacgctcctgcctatcagcagggacagaaccagctgtacaatgagctgaacctggggcgcagagaagagtacgacgtgctggatagacggagaggcagagatcctgagatgggcggcagacccagacggagaaatcctcaagagggcctgtataacgaactgcagagagacagaatggccgaggcctacagcgagatcggaatgagaggcgaacgcagaagaggcagaggccacgatggactgtatcagggcctgagcaccgccaccagagatacctatgatgccctgcacatgcaggccctgcctccaaga

SEQ ID NO: 265 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgccagcagcagcgtgtcctacatgcactggtatcagcagaagcccggacaggcccctagactgctgatctacgataccagcaagctggcctctggcatccccgccagattttctggaagcggcagcggcaccgatttcaccctgaccataagcagcctggaacctgaggacgtggccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacatctggcatgggagttggctggatcagacagcaccctggcaaaggcctggaatggatcggccacatttggtgggacgacgacaagcggtacaaccccgctctgaagtccagagtgaccatcagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcagacggagccggctgctgcacagcgactacatgaacatgacccctagacggcccggaccaaccagaagacactaccagccttacgctcctcctagagacttcgccgcctaccgcagcagagtgagattcagcagatccgccgatgctcctgcctatcagcagggacagaaccagctgtacaacgagctgaacctggggagaagagaagagtacgacgtgctggacagacggagaggcagagatcctgagatgggcggcagacccagacggagaaatcctcaagagggcctgtataatgagctgcagagagacagaatggccgaggcctacagcgagatcggaatgagaggcgagcgcagaagaggcagaggacacgatggactgtaccagggcctgagcaccgccaccagagatacctatgatgccctgcacatgcaggccctgcctccaaga

SEQ ID NO: 266 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgcctctagcagcgtgggctacatgcactggtatcagcagaagcctggacaggcccctcggctgctgatctacgatacaagcaagctggcctctggcatccccgccagattttctggaagcggcagcggcaccgatttcaccctgaccataagcagcctggaacctgaggacttcgccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacaagcacaatgggcgttggctggatcagacagcaccctggcaaaggcctggaatggatcggcttcatttggtgggacgacgacaagcggtacaaccccaacctgaagtccagagtgaccatgagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcagacggagccggctgctgcacagcgactacatgaacatgacccctagacggcccggaccaaccagaagacactaccagccttacgctcctcctcgggatttcgccgcctaccggtccagagtgagattcagcagatccgccgacgctcctgcctatcagcagggacagaaccagctgtacaacgagctgaacctggggagaagagaagagtacgacgtgctggacagacggagaggcagagatcctgagatgggcggcagacccagacggagaaatcctcaagagggcctgtataatgagctgcagagagacagaatggccgaggcctacagcgagatcggaatgagaggcgagcgcagaagaggcagaggacacgatggactgtaccagggcctgagcaccgccaccagagatacctatgatgccctgcacatgcaggccctgcctccaaga

SEQ ID NO: 267 - DNA, 1464 bp, synthetic construct
  source 1..1464: mol_type = other DNA
atggctctgcctgtgacagctctgctgctgcctctggctctgcttctgcatgccgccagacctgacatcgtgatgacacagtctcccgccacactgtctctgagccctggcgaaagagccacactgagctgcagaagctccaagagcctgcagaacgtgaacggcaacacctacctgtactggttccagcagaagcccggccagtctcctcagctgctgatctacagaatgagcaacctgaacagcggcgtgcccgacagattttctggcagcggaagcggcaccgagttcaccctgaccataagcagcctggaacctgaggacttcgccgtgtactactgtatgcagcacctggaataccccatcaccttcggagccggcaccaagctggaaatcaaaggcggcggaggatctggcggaggtggaagtggcggaggcggatctgaagtgcagctggttgaatctggcggcggactggttaagcctggcggatctctgaagctgagctgtgccgccagcggctacacctttaccagctacgtgatgcactgggtccgacaggcccctggcaaaggacttgagtggatcggctacatcaacccctacaacgatggcaccaagtacaacgagaagttccagggcagagtgaccatcagctccgacaagagcatcagcaccgcctacatggaactgagcagcctgagaagcgaggataccgccatgtactattgcgccaggggcacctactactacggcaccagagtgttcgactactggggccagggaaccctggtcaccgtgtctagcatcgaagtgatgtaccctccaccttacctggacaatgagaagtccaacggcaccatcatccacgtgaagggcaaacatctgtgcccctctccactgttccccggacctagcaagcctttctgggtgctcgttgttgttggcggcgtgctggcctgttactctctgctggttaccgtggccttcatcatcttttgggtccgaagccggcggagcagactgctgcacagcgactacatgaacatgacccctagacggcccggaccaaccagaaggcactaccagccttacgctcctcctagagatttcgccgcctacagatccagagtgcggttctccagatccgctgatgcccctgcatatcagcagggccagaaccagctgtacaatgagctgaacctggggcgcagagaagagtacgacgtgctggacagaagaagaggcagggaccctgagatgggcggcagacctagaagaagaaaccctcaagagggcctgtataacgagctgcagcgggacagaatggccgaggcctattctgagatcggcatgagaggcgagcggcggagaggtagaggacacgatggactgtatcagggcctgagcaccgccaccagagatacctacgatgccctgcacatgcaggccctgcctccaaga

SEQ ID NO: 268 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgccagcagcagcgtgtcctacatgcactggtatcagcagaagcccggacaggcccctagactgctgatctacgataccagcaagctggcctctggcatccccgccagattttctggaagcggcagcggcaccgatttcaccctgaccataagcagcctggaacctgaggacgtggccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacatctggcatgggagttggctggatcagacagcaccctggcaaaggcctggaatggatcggccacatttggtgggacgacgacaagcggtacaaccccgctctgaagtccagagtgaccatcagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagccggcggagcagactgctgcacagcgactacatgaacatgacccctagacggcccggaccaaccagaaggcactaccagccttacgctcctcctagagacttcgccgcctacagatccagagtgcggttcagcagatccgccgatgctcctgcttatcagcagggacagaaccagctgtacaacgagctgaacctggggagaagagaagagtacgacgtgctggacagaagaagaggcagggaccctgagatgggcggcagacctagaagaagaaaccctcaagagggcctctacaatgagctgcagcgcgacagaatggccgaggcctattctgagatcggcatgagaggcgagcggcggagaggtagaggacacgatggactgtatcagggcctgagcaccgccaccagagatacctatgatgccctgcacatgcaggccctgcctccaaga

SEQ ID NO: 269 - DNA, 1443 bp, synthetic construct
  source 1..1443: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgcctctagcagcgtgggctacatgcactggtatcagcagaagcctggacaggcccctcggctgctgatctacgatacaagcaagctggcctctggcatccccgccagattttctggaagcggcagcggcaccgatttcaccctgaccataagcagcctggaacctgaggacttcgccgtgtactactgcttccaaggcagcgtgtaccccttcacctttggccagggcaccaagctggaaatcaaaggcggcggaggatctggcggaggcggtagcggtggtggtggatctcaagttcagctgcaagagtctggccctggcctggtcaagcctagccaaacactgagcctgacctgtacagtgtccggcggcagcatcagcacaagcacaatgggcgttggctggatcagacagcaccctggcaaaggcctggaatggatcggcttcatttggtgggacgacgacaagcggtacaaccccaacctgaagtccagagtgaccatgagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagccggcggagcagactgctgcacagcgactacatgaacatgacccctagacggcccggaccaaccagaaggcactaccagccttacgctcctcctagagatttcgccgcctacagatccagagtgcggttcagcagatccgccgatgctcctgcttatcagcagggacagaaccagctgtacaacgagctgaacctggggagaagagaagagtacgacgtgctggacagaagaagaggcagggaccctgagatgggcggcagacctagaagaagaaaccctcaagagggcctctacaatgagctgcagcgcgacagaatggccgaggcctattctgagatcggcatgagaggcgagcggcggagaggtagaggacacgatggactgtatcagggcctgagcaccgccaccagagatacctatgatgccctgcacatgcaggccctgcctccaaga

SEQ ID NO: 270 - DNA, 1494 bp, synthetic construct
  source 1..1494: mol_type = other DNA
atggctctgcctgtgacagctctgctgctgcctctggctctgcttctgcatgccgccagacctgacatcgtgatgacacagtctcccgccacactgtctctgagccctggcgaaagagccacactgagctgcagaagctccaagagcctgcagaacgtgaacggcaacacctacctgtactggttccagcagaagcccggccagtctcctcagctgctgatctacagaatgagcaacctgaacagcggcgtgcccgacagattttctggcagcggaagcggcaccgagttcaccctgaccataagcagcctggaacctgaggacttcgccgtgtactactgtatgcagcacctggaataccccatcaccttcggagccggcaccaagctggaaatcaaaggcggcggaggatctggcggaggtggaagcggaggcggaggaagcggtggcggcggtagtggcggtggtggatctgaagttcagctggtggaatctggcggcggactggttaagcctggcggatctctgaagctgagctgtgccgccagcggctacacctttaccagctacgtgatgcactgggtccgacaggcccctggcaaaggacttgagtggatcggctacatcaacccctacaacgatggcaccaagtacaacgagaagttccagggcagagtgaccatcagctccgacaagagcatcagcaccgcctacatggaactgagcagcctgagaagcgaggataccgccatgtactattgcgccaggggcacctactactacggcaccagagtgttcgactactggggccagggaaccctggtcaccgtgtctagcatcgaagtgatgtaccctccaccttacctggacaatgagaagtccaacggcaccatcatccacgtgaagggcaaacatctgtgcccctctccactgttccccggacctagcaagcctttctgggtgctcgttgttgttggcggcgtgctggcctgttactctctgctggttaccgtggccttcatcatcttttgggtccgaagcaagcggagcagactgctgcacagcgactacatgaacatgacccctagacggcccggaccaaccagaaagcactaccagccttacgctcctcctcgggatttcgccgcctacagatccagagtgaagttctccagaagcgccgacgctcctgcctatcagcagggacagaaccagctgtacaatgagctgaacctggggcgcagagaagagtacgacgtgctggataagcggagaggcagagatcctgagatgggcggcaagcccagacggaagaatcctcaagagggcctgtataacgaactgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 271 - DNA, 1467 bp, synthetic construct
  source 1..1467: mol_type = other DNA
atggctctgcctgtgacagctctgctgctgcctctggctctgcttctgcatgccgccagacctgagatcgtgctgacacagtctcccgccacactgtcactgtctccaggcgaaagagccacactgagctgtagcgccagcagcagcgtgtcctacatgcactggtatcagcagaagcccggacaggcccctagactgctgatctacgataccagcaagctggcctctggcatccccgccagattttctggaagcggcagcggcaccgatttcaccctgaccataagcagcctggaacctgaggacgtggccgtgtactactgcttccaaggcagcgtgtaccccttcacctttggccagggcaccaagctggaaatcaaaggcggcggaggatctggcggaggaagcggaggcggttcaggtggtggtggtagtggtggcggtggatctcaagtccagctgcaagagtctggccctggcctggttaagcctagccagacactgagcctgacctgtacagtgtccggcggcagcatcagcacatctggcatgggagttggctggatcagacagcaccctggcaaaggcctggaatggatcggccacatttggtgggacgacgacaagcggtacaaccccgctctgaagtccagagtgaccatcagcgtggacaccagcaagaaccagttcagcctgaagctgagcagcgtgacagccgccgataccgccgtgtattattgcgcccggatggaactgtggtcctactacttcgactactggggccagggaaccctggtcaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccgcagcagagtgaagttcagcagatccgccgatgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaaga

SEQ ID NO: 272 - DNA, 1464 bp, synthetic construct
  source 1..1464: mol_type = other DNA
atggcactgcccgtgaccgctctgctcctgccccttgctttgctccttcacgccgcccggcctgatatcgtcatgacccagagtcctgccactctgtcactctctccaggtgagagggctacactctcttgccgctcttccaagtccctccagaacgtgaatggcaatacctatctgtactggttccagcagaagcccgggcagtcaccacagctcctcatctaccgcatgagcaacctcaactcaggcgtccccgatcggttctcaggttcaggatctggtaccgagttcacccttactatcagttccctggaacctgaagacttcgctgtctactactgtatgcagcacctggaatacccaatcaccttcggggcaggtactaagctcgagatcaaaggcggcggagggtccggcggcggtgggagtggtggtggtggaagtgaggtgcagctggtcgagagcggtggtggcctggtcaaaccaggcggttcacttaagctgtcctgcgcagcctccggttacacattcacatcctacgttatgcactgggttcggcaggctcccggcaagggcctcgagtggatcggatatattaatccatacaatgacggtaccaaatacaacgagaaattccagggcagggtgacaatttcttctgataaaagtatctcaaccgcttacatggaactcagcagcttgcgctccgaggatactgctatgtattattgcgcacgcgggacatattactacgggacacgggttttcgattactggggtcaaggcaccttggttacagtcagcagcattgaggtgatgtatccacctccatatctcgacaatgaaaagtcaaacgggactattatccatgtcaagggaaagcacttgtgcccttcccctctgtttccaggtccaagcaagccattttgggtgctcgttgtcgtcggaggcgttctcgcctgctactccctgctcgttaccgttgcattcatcattttctgggtgaggtccaaaagaagccggctcttgcacagcgattatatgaacatgacccctaggcggcctggccctactagaaagcactatcagccctatgcaccaccacgcgacttcgcagcatacagaagccgggtgaagttttctcgcagtgcagacgctccagcttaccagcaaggccaaaaccagctttacaacgagctgaacctcggtagacgggaggagtacgacgtgctggacaaacgccggggacgggatccagaaatgggaggcaaaccaaggcggaagaatccccaagagggtctgtacaacgagctccagaaggacaagatggcagaggcatattcagagatcggtatgaagggagagcgcaggaggggtaagggacacgacggcttgtaccaggggctttccactgccacaaaggacacctacgacgccctgcacatgcaggccttgcctccccgg

SEQ ID NO: 273 - DNA, 1464 bp, synthetic construct
  source 1..1464: mol_type = other DNA
atggctctgcccgtgaccgctcttctcctccctcttgctcttttgctccatgccgccaggcctgacatcgtcatgacccaatctcccgctaccttgagcctgtcacccggcgagcgggcaaccctgtcttgcaggtccagcaagtcactccaaaacgttaacggcaacacctacctgtactggttccaacagaagcctggacagagccctcagctcctgatctacaggatgtccaacctgaattccggggtgcccgatcgcttttccggctccggctccggtaccgagtttactttgaccatctcctcattggagccagaggattttgcagtgtattactgcatgcagcacctggagtaccctatcaccttcggggctgggaccaagctggagatcaaaggcggaggcggttccggtggtggtggttcaggcggcggtgggtctgaggttcagctcgttgagagcggcggcggtcttgtcaaacccggcggttccctgaagctctcatgcgctgcttctggatacacattcacctcctacgttatgcattgggtgcggcaggctccaggaaaggggctcgaatggatcgggtatatcaacccatacaatgatgggaccaagtacaacgagaagttccagggcagggtcaccatctcttctgataagtccatttccaccgcttacatggagctgtctagcctgagatccgaggacacagctatgtactactgtgccagaggcacatactactacgggacccgggttttcgactattggggtcagggaacccttgtcaccgtgtctagcatcgaggtgatgtatcctccaccatacctggataacgagaagtccaatgggaccattatccacgttaaagggaagcacctctgtccttcccctctgtttcccggaccttccaagcccttctgggtgctcgtggtggtcggtggcgtccttgcttgctatagcctgctggtgaccgtggctttcattatcttctgggtgagaagcaagcgcagccggctcctccactcagactacatgaatatgaccccacggcgccccggacccactcgcaaacactatcaaccctacgctcctcctcgggacttcgctgcttatcgctcccgggtcaagttctcccgctctgcagacgccccagcctatcaacaggggcagaaccagctttataacgaactcaacctggggcggagggaggaatacgatgtgcttgacaaaagacggggtcgggatcctgagatgggcggtaagcccagacggaagaatccacaggaggggctgtacaacgaactgcagaaggacaagatggccgaggcctactctgagatcggcatgaaaggggagcgccgccgcggtaagggccatgatggtctctatcagggcttgtccaccgctactaaggacacatacgatgccctgcacatgcaggcactgcctccccgc

SEQ ID NO: 274 - DNA, 1464 bp, synthetic construct
  source 1..1464: mol_type = other DNA
atggccctccccgtgacagccctcctgctccctctggcccttctcttgcatgccgctagacccgatattgtcatgacccagtcaccagccactttgtctctctcacccggtgaacgcgccaccttgtcctgccggagttccaagagcctccaaaatgtgaatggtaatacttacctctactggttccagcaaaagcctgggcaatcacctcagctgctgatttatcggatgtccaaccttaactcaggggttcccgatcgcttctcaggtagcgggagcgggaccgagttcacactcacaattagcagcctggagcccgaggactttgcagtgtactactgcatgcaacacctggagtaccccatcacattcggagcaggaaccaagttggagatcaaaggtggtggtggttccggtggtggtggctctggtggtggtggctcagaagtccagcttgtcgagagcggaggaggccttgttaagcccggagggtctctcaagctgagctgcgccgcatccggctatacattcacctcctacgtgatgcattgggtgcgccaagcacccggcaaaggtctggagtggattgggtacatcaacccctacaacgacggcaccaaatacaacgagaagttccagggcagagttaccatcagcagcgacaagtcaatcagcaccgcctacatggagctctcatccctgcggtccgaggatacagctatgtattattgcgcccggggaacctactactacgggacacgcgtctttgactattggggacaggggactcttgtgactgtttcctcaatcgaggtcatgtacccaccaccttacctcgataacgagaaaagtaacggtaccattatccacgtgaagggaaagcatctgtgtccttccccactctttcctggacccagcaagccattctgggtgctcgtcgtcgtgggcggagttctggcatgctactcactcctggtgaccgttgccttcattatcttttgggtgcgctccaagcgctcccgcctcctccacagcgactacatgaacatgacccctcggcggcccggccctacccgcaaacactatcagccatacgctcctcctcgcgacttcgctgcttaccggagccgcgtcaaattcagtcggtctgccgatgcccctgcataccagcagggtcagaaccagctgtataacgagctgaacctcggccggcgcgaagaatacgatgtcctggataaacggcgcggtagagatcccgagatgggtgggaagccacgccggaagaatccacaggaagggctttacaacgaactgcagaaggataagatggccgaggcatactctgagatcgggatgaaaggtgaacggagaaggggaaagggccatgatggcctctaccaaggcctgtccacagctaccaaggacacctacgacgccctgcacatgcaggccttgcctccccgc

SEQ ID NO: 275 - DNA, 1443 bp, synthetic construct
  source 1..1443: mol_type = other DNA
atggcccttccagtcacagccctgttgctgccactggcacttcttcttcacgccgccagacccgagattgtgctcacacaatcacctgctactctctccctctcacccggggagcgcgcaaccctctcatgctccgctagtagtagcgtgagctacatgcactggtatcagcagaaacccggccaggctccccggctgttgatctacgacacttcaaaactggcttccgggattcccgcacggtttagcggaagcggtagcggcaccgacttcacccttactatctcctctctcgaaccagaggacgtggctgtctattactgctttcaaggttccgtgtaccccttcactttcgggcaggggaccaagctggagatcaagggtggtggtggctccggcggtggaggtagcggcggtggagggagtcaggtccaactccaggaatcaggccctggtctcgtgaaaccctctcagaccctgagcctgacctgcacagtctccggtgggtccatttccacctccggcatgggtgtgggctggattcgccagcaccctgggaaggggctggagtggatcgggcacatctggtgggacgatgataaacgctacaatcctgccctgaagtcccgggtgaccatctccgtggacacatctaagaatcagttttctctgaagctctcatccgttacagccgctgacaccgcagtgtattattgcgctcggatggagctctggtcttattactttgattactggggccaagggactctcgtgacagtgtcatccatcgaggtcatgtatccaccaccatacctggacaatgagaaatccaatgggactatcatccatgtgaaggggaagcacctctgtccctcaccactgttccctgggccctccaagcctttctgggttctcgtggtcgtcggaggggtcctggcatgctatagcctgctggtgaccgttgcatttattattttctgggtccggagcaagcggtctcggctgctccattccgattacatgaatatgacaccccggagacctggccctaccaggaaacactaccagccctacgctcctccccgcgacttcgctgcttacagaagccgcgttaagttcagtcggtccgctgatgcccctgcctaccagcagggccagaatcagctctacaatgagctcaacttgggtagacgggaagagtacgatgtgctcgacaagcggagagggagagatcctgagatgggaggtaagccacggagaaagaatccacaggagggtctgtacaacgaactccaaaaggataaaatggccgaggcatacagcgaaatcggcatgaaaggcgaaaggaggagaggaaagggccatgatgggttgtaccaggggctcagcactgctactaaggacacctacgacgccttgcacatgcaggcactgccaccccgc

SEQ ID NO: 276 - DNA, 1443 bp, synthetic construct
  source 1..1443: mol_type = other DNA
atggccttgcccgttaccgcattgcttctccctctcgccctcctgctgcatgccgcacggcctgagattgtcctgactcaatcacccgccacactgagcctgtctcctggcgagagggctaccctcagttgctctgctagttccagtgtctcctacatgcactggtatcaacaaaagcccggccaagcccctagactgctcatctacgacactagcaagctggcatctgggatccccgcacggttcagcgggagcggtagcggcactgactttacccttaccatcagctctctggaaccagaggacgtggccgtctactactgcttccagggcagtgtgtaccccttcaccttcggccaggggaccaaactggagattaaaggtggtggcggctccggtggaggaggctccggcggtggcggttcccaagttcagcttcaggagtcagggccaggcttggtgaagcctagtcagacattgtccctcacatgcaccgtttcaggagggagtatttctacctccggaatgggtgtgggttggatccggcagcatcctggcaagggcctcgaatggatcggccatatctggtgggatgacgacaagcgctataacccagcacttaagagccgggtcaccatctctgtggacacctccaagaaccagttctccctcaaactgagttccgtgacagccgccgacactgctgtctattattgcgcccggatggaactttggtcatactattttgactactggggccagggcactctggtgactgtctccagtatcgaggtgatgtaccctcctccatatctggataatgagaagtccaacggcaccatcatccatgttaagggaaaacacttgtgcccctctcccttgttccccggaccctccaagcccttctgggtgctcgttgtggtcggaggcgtgcttgcctgctactccctgctggttactgtcgccttcatcatcttctgggttcggagcaagcgctcccgcttgctgcattccgactacatgaatatgactcctcggcggcctggtcctacccggaagcactaccagccttacgcaccaccaagggattttgctgcttaccgcagccgcgtgaaattttcccggtctgccgatgcacccgcctaccagcaaggacaaaatcagctgtacaacgaactcaatctcggacgccgcgaggagtacgacgtcttggacaagaggcggggtagagatcctgagatgggtggcaagccacggcgcaagaatccacaagaaggcctctacaacgagctgcagaaggacaagatggctgaggcatatagcgagattggcatgaaaggagagcgccgcagaggaaagggccacgatggcctctaccaaggtctgtccaccgctaccaaggacacatacgatgccctccacatgcaggccctcccaccccgg

SEQ ID NO: 277 - DNA, 1443 bp, synthetic construct
  source 1..1443: mol_type = other DNA
atggcactgcccgtgaccgctcttttgttgcctctcgcactgctgcttcacgctgcccgccccgaaattgtcctcacccaaagtccagcaaccctcagtctgtctccaggtgagagggctacccttagttgcagtgccagctcctctgtgtcctatatgcactggtaccagcagaagccaggccaagcaccccgcctcctgatctatgatacctcaaaactggcctcaggtattcccgctcggttcagcggaagcggtagcggcacagactttaccctcaccatttcctcactcgaaccagaggacgtcgccgtctattattgcttccaaggatctgtgtaccctttcactttcggccaggggacaaaactggagatcaaaggtggtggtgggtcaggtggaggaggcagcggcggaggtggctcccaggtgcagctgcaagaaagcggccctgggctggtcaagccctctcagaccctttccctcacatgtacagtttccggtggtagcatctcaacctccgggatgggtgtcggatggatccgccaacatccagggaaaggcttggagtggatcggccatatttggtgggatgacgacaagaggtacaacccagctctgaagtcccgggtcactatctctgtggatactagcaagaaccagttcagtctcaagctgagtagcgtcactgcagctgatacagctgtctactattgcgcacgcatggagctgtggtcctactacttcgactattggggacagggcaccctcgtcaccgtttcctcaattgaggtgatgtatcctcctccatatctcgacaacgagaaatccaacgggacaattatccacgtgaagggtaagcatctgtgtcccagccccttgttcccaggccctagcaagcctttctgggtgcttgtcgtggtgggaggcgtccttgcatgttatagcctcctggtcaccgttgcatttatcattttctgggtgaggtctaagagaagcaggctcctgcatagtgactatatgaacatgacccctcggcggcctggaccaacccgcaaacactatcaaccctatgctccaccccgggattttgccgcctacagatctagggtgaaattttcccggtctgcagacgctccagcctaccagcaggggcagaaccaactctacaacgagttgaatctggggcgccgggaggagtatgacgtccttgataagcgcagaggtagggaccctgagatgggcggaaagcccaggaggaagaaccctcaagaggggttgtacaatgagctccagaaagataagatggccgaagcctactcagagattggtatgaagggtgaacgccgcaggggtaagggacacgacgggctttaccaggggttgtcaactgctaccaaggatacctacgacgctctccacatgcaggcccttcctccccgg

SEQ ID NO: 278 - protein, 498 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 279 - protein, 493 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 280 - protein, 482 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 281 - protein, 492 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 282 - protein, 486 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 283 - protein, 497 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 284 - protein, 492 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 285 - protein, 484 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 286 - protein, 488 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 287 - protein, 491 aa, synthetic construct
MLLLVTSLLLCELPHPAFLLIPEIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIKGSTSGSGKPGSGEGSTKGEVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 288 - protein, 488 aa, synthetic construct
MALPVTALLLPLALLLHAARPDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 289 - protein, 481 aa, synthetic construct
MALPVTALLLPLALLLHAARPQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSGGGGSGGGGSGGGGSENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 290 - protein, 481 aa, synthetic construct
MALPVTALLLPLALLLHAARPEIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKGGGGSGGGGSGGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 291 - protein, 483 aa, synthetic construct
MALPVTALLLPLALLLHAARPEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 292 - protein, 483 aa, synthetic construct
MALPVTALLLPLALLLHAARPQVQLVQSGAEVKKPGSSVKVSCKASGHTISSYAYSWVRQAPGQGLEWMGDIIPAYGSPNYAQKFQGRVTITADESTSTAYMELSSLRSEDTAVYYCAREDFGKNYAMDVWGQGTLVTVSSGGGGSGGGGSGGGGSEIVLTQSPGTLSLSPGERATLSCRASQHVSSHYLAWYQQKPGQAPRLLIYGASSRATGIPDRFSGSGSGTDFTLTISRLEPEDFAVYYCQHYGQSQFTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 483 aa, synthetic construct
MALPVTALLLPLALLLHAARPEIVLTQSPGTLSLSPGERATLSCRASQHVSSHYLAWYQQKPGQAPRLLIYGASSRATGIPDRFSGSGSGTDFTLTISRLEPEDFAVYYCQHYGQSQFTFGQGTKVEIKGGGGSGGGGSGGGGSQVQLVQSGAEVKKPGSSVKVSCKASGHTISSYAYSWVRQAPGQGLEWMGDIIPAYGSPNYAQKFQGRVTITADESTSTAYMELSSLRSEDTAVYYCAREDFGKNYAMDVWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 294 - protein, 488 aa, synthetic construct
MALPVTALLLPLALLLHAARPDIVMTQTPLSLSVTPGQPASISCKSSQSLETTTGTTYLNWYLQKPGQSPQLLIYRASKRFSGVPDRFSGSGSGTDFTLKISRVEAEDVGVYYCLQLLEDPYTFGCGTKLEIKGGGGSGGGGSGGGGSQVQLVQSGAEVKKPGASVKVSCKASGYDFTDYIMHWVRQAPGQCLEWMGYINPYNDGSKYTDKFQERVTMTSDTSISTAYMELSRLRSDDTAVYYCARGTYYYGPELFDYWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 481 aa, synthetic construct
MALPVTALLLPLALLLHAARPEIVLTQSPATLSLSPGERATLSCSASSSVGYMHWYQQKPGQAPRLLIYDTSKLASGIPARFSGSGSGTDFTLTISSLEPEDFAVYYCFQGSVYPFTFGQGTKLEIKGGGGSGGGGSGGGGSQVQLQESGPGLVKPSQTLSLTCTVSGGSISTSTMGVGWIRQHPGKGLEWIGFIWWDDDKRYNPNLKSRVTMSVDTSKNQFSLKLSSVTAADTAVYYCARMELWSYYFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - protein, 488 aa, synthetic construct
MALPVTALLLPLALLLHAARPDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSIEVMYPPPYLDNEKSNGTIIHVKGKHLCPSPLFPGPSKPFWVLVVVGGVLACYSLLVTVAFIIFWVRSRRSRLLHSDYMNMTPRRPGPTRRHYQPYAPPRDFAAYRSRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 297 - protein, 481 aa, synthetic construct
MALPVTALLLPLALLLHAARPEIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKGGGGSGGGGSGGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSIEVMYPPPYLDNEKSNGTIIHVKGKHLCPSPLFPGPSKPFWVLVVVGGVLACYSLLVTVAFIIFWVRSRRSRLLHSDYMNMTPRRPGPTRRHYQPYAPPRDFAAYRSRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 298 - protein, 481 aa, synthetic construct
MALPVTALLLPLALLLHAARPEIVLTQSPATLSLSPGERATLSCSASSSVGYMHWYQQKPGQAPRLLIYDTSKLASGIPARFSGSGSGTDFTLTISSLEPEDFAVYYCFQGSVYPFTFGQGTKLEIKGGGGSGGGGSGGGGSQVQLQESGPGLVKPSQTLSLTCTVSGGSISTSTMGVGWIRQHPGKGLEWIGFIWWDDDKRYNPNLKSRVTMSVDTSKNQFSLKLSSVTAADTAVYYCARMELWSYYFDYWGQGTLVTVSSIEVMYPPPYLDNEKSNGTIIHVKGKHLCPSPLFPGPSKPFWVLVVVGGVLACYSLLVTVAFIIFWVRSRRSRLLHSDYMNMTPRRPGPTRRHYQPYAPPRDFAAYRSRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 299 - protein, 498 aa, synthetic construct
MALPVTALLLPLALLLHAARPDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 300 - protein, 489 aa, synthetic construct
MALPVTALLLPLALLLHAARPEIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKGGGGSGGGSGGGSGGGGSGGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 301 - DNA, 1446 bp, synthetic construct
  source 1..1446: mol_type = other DNA
atggcactcccagtcaccgcacttctgctgcctctcgccctgctgctccatgcagccagacccgacatccagatgacccaaaccaccagctccctgtccgcttccctgggtgaccgggtgactatctcttgccgggcctcccaagacatctccaagtacctgaactggtatcagcaaaagcctgacggcaccgtcaagctcctcatctaccatacctccagactgcactccggggtgcctagcaggttcagcggaagtgggagcggcaccgactacagcctcaccatctccaacctggagcaggaggacatcgccacctacttctgccagcaggggaacacactgccctacaccttcggcggtggcaccaagctggagatcacaggtggcggaggttccggaggaggaggtagtggaggtggaggcagcgaggtgaagctccaggaatccggaccaggtctggtggctcccagccagtccctcagcgtgacctgcaccgtgagcggcgtgtctcttcccgattacggagtgtcctggatcagacagccaccccggaagggtctggagtggctgggagtgatctggggttccgagaccacatactacaactcagccctcaagagccggctcaccatcatcaaggataactccaagtcccaggtcttcctgaagatgaactctctccagaccgacgacaccgccatctactactgcgccaagcactactactacggcgggtcctacgccatggactactggggtcagggaacctccgtcaccgtcagctctatcgaggtgatgtaccctcctccctacctcgacaacgagaagagcaacggcaccatcatccatgtgaaggggaagcatctctgcccctcacccctgttccccggaccatccaagccattctgggtgctggtggttgttggtggggtcctggcttgctactcactcctggtcaccgtcgccttcatcatcttctgggtgcggtcaaagaggtcccggctcttgcactccgattacatgaacatgactccaaggaggcctggtcccacacggaagcactaccaaccatatgccccaccacgcgacttcgctgcttaccggagccgggtcaagttcagtcggagtgcagacgccccagcctaccagcagggccagaaccaactctacaacgagcttaatctgggtcgccgggaggagtatgacgtgctcgataagagaaggggccgggatcctgagatgggcggtaagcccagacggaagaaccctcaggaggggttgtataatgagctccagaaggacaagatggccgaggcatactccgagatcggcatgaaaggtgagcggaggagaggcaaggggcatgacggcctgtaccaggggctcagcacagccaccaaggatacctatgacgcactccacatgcaggcactgcctccacgg

SEQ ID NO: 302 - DNA, 1446 bp, synthetic construct
  source 1..1446: mol_type = other DNA
atggcccttcccgtcaccgctctcctcctgccactggccttgctgctgcacgctgcacggccagacatccagatgacccagacaaccagctctctgtcagcctctctcggcgatcgcgtcacaatcagctgccgcgcttcccaagacatctccaagtacctgaactggtaccagcaaaagcccgacggcaccgtgaagctgctcatctaccacacctccagactgcatagcggggtgcccagcagattcagtggctcaggctcaggcaccgactacagcctgaccatctccaacctggagcaggaggacattgccacatacttctgccagcagggcaacaccctgccctacaccttcggaggcggcacaaagctggagatcaccggtggaggagggagtggaggaggaggcagtggtggcggaggttccgaggtgaagctccaggaatcaggtccaggactggtcgccccttcccagtccctgtccgtcacctgcaccgtgagtggcgtcagcctcccagactacggtgtgtcttggatccgccaacctcctcgcaaaggcctggaatggctcggcgtcatctggggaagcgagacaacctactataactccgcactgaagtcccgcctcaccatcatcaaggataatagcaagagccaggtcttcctcaagatgaactccctgcagaccgacgataccgccatctactactgtgccaagcactactactacggaggttcttacgccatggattactggggacagggaacctctgtcaccgtcagctccatcgaggtcatgtatccaccaccctacctggacaacgaaaagagcaatggcaccatcatccacgtgaaggggaagcacctctgcccctcacccctgttccctggtccctccaagcctttctgggtcctggtcgtcgtgggaggcgtgttggcctgttactccctgctcgtcaccgtcgccttcatcatcttctgggttaggagtaagcggtcccggcttctgcactctgactacatgaacatgacacccagaagacctgggccaacccggaagcactaccagccctacgctccacccagggactttgcagcctacaggtcccgcgtcaagttctcccggtctgctgacgcacctgcctaccagcagggccaaaaccagctctacaacgagttgaacctcggcagacgggaggagtacgacgtcctcgacaaaaggcggggtcgggatcctgagatgggcggtaagccaaggcggaagaacccacaggaaggcctctataatgagctccagaaggataagatggctgaggcctactccgagatcgggatgaagggcgaaaggagacggggtaaggggcacgacggcctctatcagggtctgagcaccgccaccaaggacacctacgacgccctgcacatgcaggcactgccacctcgg

SEQ ID NO: 303 - DNA, 1446 bp, synthetic construct
  source 1..1446: mol_type = other DNA
atggctctgccagtgaccgcactgctgctgcccttagccttactccttcacgcagccaggcccgacatccagatgacccagaccaccagctccctttccgcaagcctcggcgacagggtcaccatctcctgtcgggccagccaggacatcagcaagtacctgaactggtaccagcagaagcccgacggcaccgtgaagctgctgatctaccacacctcacggctgcactcaggcgtgccctcacggtttagcggatcaggcagcggcaccgactacagcctgactatcagcaacctggagcaggaggacatcgccacctacttctgccagcagggcaacaccctgccctacaccttcggaggcggcaccaagctggagatcaccggtggcggtggttcaggtggcggaggctcaggaggaggcggcagcgaggtgaagctgcaggagtcaggtccaggactggtggcacccagccagagcctgagcgtgacttgcaccgtgtcaggcgtgagcctgccagactacggcgtgagctggatccggcagcctcctcggaagggcttagagtggctgggcgtgatctggggcagcgagaccacctactacaactcagccctgaagagccggctgaccatcatcaaggacaacagcaagagccaggtgttcctgaagatgaacagcctgcagaccgacgacaccgccatctactactgcgccaagcactactactacggcggcagctacgccatggactactggggacagggtaccagcgtgaccgtgagcagcatcgaggtgatgtaccctcctccctacctggacaacgagaagagcaacggcaccatcatccacgtgaagggcaagcacctgtgccctagccctttattccccggcccctcaaaacccttctgggtgctggtcgtcgtcggtggcgtgctggcatgctacagcctgctggtgaccgtggccttcatcatattctgggtccggtcaaagcggagccggttactgcacagcgactacatgaacatgactccacggcgtccaggtcccactcggaagcactaccaaccctacgctcctccccgtgactttgctgcctaccgtagccgggtgaagttctccaggagcgccgatgccccagcctaccagcagggccagaaccagctctacaatgagcttaaccttggcaggcgggaggagtacgacgtgctggacaagaggaggggccgtgatcccgagatgggaggcaagccccgtaggaagaatccccaggagggcctttacaacgagctccagaaggacaagatggccgaggcctacagcgagatcggcatgaagggagagcgtaggcgtggaaagggccacgacggcctgtaccagggcctgagcactgctaccaaggacacctacgacgccctgcacatgcaggctcttccaccccgg

SEQ ID NO: 304 - DNA, 1467 bp, synthetic construct
  source 1..1467: mol_type = other DNA
atgctcctcctggtgaccagcttgctcctgtgcgaactgccacaccccgccttcctcctcatccccgatatccagatgacccagaccacctcctccctgagcgcaagcctcggcgatcgggtgaccatctcatgcagggcctcccaggacatctccaagtatctgaactggtatcagcagaagcctgacggcaccgtcaagctgctcatctaccacacctcacggctgcactcaggcgtcccctcaagattcagcggtagcggatccgggaccgactactcccttaccatcagcaacctggagcaggaggatatcgccacatacttctgccagcagggtaacaccctgccctataccttcggcggtgggaccaagctggagatcaccggttctacatccggatccggcaagcctggtagtggcgagggctccaccaaaggggaggtgaagctgcaggagtccggtccaggtctggtggctccaagtcagtccctgtctgtgacttgcaccgtgtcaggcgtgagcctgcctgactacggggtgagctggatccggcagccacctcggaaggggttggagtggctgggagtcatctggggatccgagaccacctactacaattccgccctcaaaagccgcctcaccatcatcaaggacaactccaagtcccaggtcttcctgaagatgaattccctgcagaccgacgacaccgctatctattactgcgccaagcattactactacggcgggtcctacgccatggactactggggtcaaggcacctccgtcactgtttcctccgcagcagccatcgaggtcatgtatcctcctccctacctcgacaacgagaagtccaacgggaccatcatccacgtgaagggcaagcacctctgcccaagcccactgttcccagggccctccaaaccattctgggtgctcgtggtggtgggtggcgtgctcgcttgctactccctcctggtcaccgtcgccttcatcatcttttgggtccggagtaagcgcagccgcctgctccatagcgactacatgaacatgaccccacggagacctggtcccacccggaaacactaccagccctacgcaccacccagggacttcgctgcctatcggtcccgggttaaattctctaggtccgctgatgccccagcctaccagcagggccagaaccagctgtacaatgagctgaacctgggtagacgggaggagtatgacgtcctggataagcgcagagggagagaccccgagatgggtggaaagcccaggcggaagaatccccaggagggtctctataacgagctccagaaggacaagatggccgaggcctacagcgagatcgggatgaaaggggaaagaaggcggggaaagggccatgacggactgtaccagggtctgtccaccgctaccaaggacacctacgatgcactgcacatgcaggcactgcctcctcgg

SEQ ID NO: 305 - DNA, 1467 bp, synthetic construct
  source 1..1467: mol_type = other DNA
atgctgcttctcgttacatctctgttgctctgcgagctgcctcatccagccttcctcctgattcccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 306 - DNA, 1464 bp, synthetic construct
  source 1..1464: mol_type = other DNA
atggcacttccagttacagcacttctgcttccattggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 307 - DNA, 1464 bp, synthetic construct
  source 1..1464: mol_type = other DNA
atggcactgcccgtcaccgcactcctgctcccactggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggcagcacctcaggctctgggaagcctggcagcggtgaaggcagcaccaagggtgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccgctgccgctatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 308 - DNA, 1446 bp, synthetic construct
  source 1..1446: mol_type = other DNA
atggcacttccagttacagcacttctgcttccattggcactgctgctccatgcagctcgccccgatatccagatgacccagaccacctctagcctcagcgcctctctgggtgaccgcgtcaccatctcttgccgggccagccaagacatctctaagtacctgaactggtaccagcagaaacctgacggaaccgtgaagctgctgatctaccacaccagtcggctgcattccggggtgccttccaggttcagcggttccggctctgggaccgattatagtctcaccatctccaacctcgagcaggaggacatcgcaacctacttctgccagcaggggaacaccctgccctacaccttcggtggcgggaccaagctggagatcactggaggtggtggcagcggaggtggaggatcaggtggaggcggtagcgaggtgaagctgcaggagtccggacctggtctggtggccccaagccagtccctcagcgtcacctgcacagtgtccggggtgtccctgcctgactacggtgtctcctggatcaggcaaccaccccggaagggtctcgagtggctgggcgtcatctggggctccgagaccacctactacaacagcgctctgaagtcccggctgaccatcatcaaagacaactccaagagccaggtgttcttgaagatgaactccctgcaaaccgatgacaccgccatctactactgcgccaagcactactactatggcggtagctacgccatggattattggggtcagggcaccagtgtcaccgtctcctccatcgaggtgatgtaccctccaccctatctggacaacgagaagtccaacggcaccatcatccacgtgaagggcaagcacctgtgccctagccctctgttcccaggaccctccaagcccttctgggtgctggtcgtggtgggaggagtcctggcctgctattccctcctcgtcaccgtggcatttatcatcttctgggtccggagcaagcggtcacgcctgctccactccgactacatgaacatgactcctcgcagacctggacccacccggaagcactaccagccttatgccccaccccgcgactttgccgcttaccgctctcgggtcaagttctctcggtcagcagacgcccctgcataccagcagggccagaaccagctgtataacgagctgaacctcggcagacgggaggagtacgatgtgctggacaagaggagaggcagagaccccgagatgggtggtaagccacggcgcaagaacccacaggagggcttgtacaacgaactgcagaaggacaagatggccgaggcctacagcgagatcggcatgaagggagagaggcgcaggggcaagggtcacgacggcctgtaccaagggctgtccaccgcaaccaaggacacctacgatgccctgcacatgcaggccctcccaccaagg

SEQ ID NO: 309 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 310 - protein, 488 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 311 - DNA, 1425 bp, synthetic construct
  source 1..1425: mol_type = other DNA
atgggcgtgcctacccagctgctcggtctcctgctgctctggatcaccgacgctatctgcgacatccaaatgacccagagtcccgcttccctcagcacctccctgggtgagaccgtcaccatccagtgccaggcatccgaggacatctacagtggtctcgcctggtaccagcagaagcctggtaagtcccctcagctgctgatctacggtgcttccgatctgcaggacggagtccctagccgcttctcaggctctggctccggtacccagtactccctgaagatcacatccatgcagaccgaagacgagggagtgtacttctgccagcaggggctgacctaccctcggacattcggcggtggaaccaagctcgagctgaagggaggtggaggcagtggtggcggaggatctggtggtggtggctccgaggtccaactgcagcagtccggcgctgagctggtgaggcccggaaccagcgtcaaactcagctgcaaggtgagcggggacaccatcaccttctactacatgcacttcgtcaagcagaggcctgggcagggtcttgaatggatcggccggatcgatccagaggacgagtctacaaagtactccgagaagttcaagaacaaagcaaccctgaccgccgacacaagctccaacaccgcctacctgaagctgtccagcctcacctctgaggacaccgccacctacttctgcatctacggcgggtactacttcgactattggggccaaggggtgatggtcaccgtgtcctctatcgagttcatgtatcctcctccctacctggacaacgagcggagcaacggcaccatcatccacatcaaagagaagcacctctgccacacccaatcctctcccaaactcttctgggccctcgttgtggtcgcaggcgtgctcttctgctacgggctgctggtgactgtggccttgtgcgtgatctggaccaacagtagacggaatcggggaggtcagagcgactacatgaacatgacacctcgcagaccaggcctgacacggaagccctaccaaccatacgctcctgcccgggattttgcagcatatcggccacgggccaagtttagcaggtccgcagagaccgcagccaacctgcaagaccctaaccagctgtacaacgagctgaaccttggtcgccgggaggagtacgacgtcctggagaagaagagagcacgggatcccgagatgggcggaaagcaacaacgccggcggaatcctcaggagggtgtctacaacgccctccagaaggacaagatggctgaggcctactccgagatcggcactaagggcgagcgcagacggggaaagggtcacgacgggctgtaccagggtctcagcaccgcaaccaaggatacctacgacgccctgcacatgcaaaccctcgcaccccgg

SEQ ID NO: 312 - DNA, 1095 bp, synthetic construct
  source 1..1095: mol_type = other DNA
atggctctccccgtgaccgctctcctccttcccttggccttgttgctgcacgccgcaaggcctcaggtgaagctggaggaatccggaggcggactcgtccaagcaggtcggtccctcaggctgtcttgcgctgccagcgagcacaccttctctagccacgtgatgggttggttcagacaggccccaggaaaggagcgggaatccgtggcagtgatcggctggcgggacatcagcacctcctacgccgactccgtcaagggccggttcaccatcagccgcgacaacgccaagaagaccctgtacctgcagatgaacagcctcaagcctgaggacaccgccgtgtactactgcgccgccagaaggatcgacgccgcagacttcgactcctggggtcagggaacccaggtgaccgtgtcctccacctctaccaccacaccagcacccagacctcctactcccgctcccaccatcgcttcccagcccctgtccctcagacccgaagcctgcagaccagcagctggcggtgcagtgcacaccaggggtcttgacttcgcctgtgacatctacatctgggctccactggctgggacttgcggcgttctgctgctgagcctggtgatcaccctgtactgcaagcggggccggaagaagctgctctacatcttcaagcagcctttcatgcggcccgttcagaccacccaggaggaagacgggtgcagttgccgcttccctgaggaggaggagggaggatgcgagctgcgggtcaagttctctcggtccgctgatgccccagcctaccagcagggccagaaccagctctataacgagctgaacctcggtaggcgggaggagtacgacgtcctggacaaaaggaggggacgggatcctgagatgggaggaaagccacggcggaagaaccctcaagaggggctgtacaacgaactccagaaggacaagatggctgaggcttattctgagatcggcatgaagggagagcgcagacgcggcaagggacacgatggcctgtatcagggactgagcaccgccaccaaggatacctacgacgccctccatatgcaggctctcccaccacgg

SEQ ID NO: 313 - DNA, 1092 bp, synthetic construct
  source 1..1092: mol_type = other DNA
atggcactgcccgtcacagctctgctgctgcccttggccctcttgttgcacgcagcacggccagaggtgcaacttgtcgagagcggtggtggccttgtgcaggccggaggcagtttgcgcctcagttgtgcagcttctggccggaccttcaccatgggctggttcagacaggcccctggtaaggaacgcgagttcgtggccgccatcagcctgtccccaaccctggcctactacgcagagagcgtgaagggtcggttcaccatctcccgcgacaacgcaaagaacaccgtggtgctccagatgaactccctgaagccagaagacaccgccctgtactactgcgcagccgaccggaagagcgtgatgtccatccgccctgactactggggccaaggcacacaggtcacagtgtccagcacctccactaccactccagctccacgccctccaacacccgcaccaaccatcgccagccagcctctgagtctgagacccgaagcatgccggccagctgctggaggtgccgtgcacaccagagggctggacttcgcctgcgacatctacatctgggctcctctggccggaacttgcggggtgctgctcctctcactggtcatcaccctgtactgcaagaggggcaggaagaagctcctgtacatcttcaagcagcccttcatgcggccagtccagacaacccaggaggaagacggatgcagctgtcgcttccccgaggaggaggaaggcggctgcgaattgcgggtcaagttcagcagatccgctgacgctcctgcctaccaacagggacagaaccagctctacaacgagctgaacctgggaaggcgggaggagtacgacgtcctggacaagagaagaggacgcgaccccgagatgggaggtaagcccagacgcaagaaccctcaagagggactgtataacgagctgcagaaggacaagatggccgaggcctacagcgagatcggcatgaagggtgaaagaagacggggaaaggggcacgacggtctgtatcaggggctctccaccgcaaccaaggatacctatgacgctctgcacatgcaggcactccctccacgc

SEQ ID NO: 314 - DNA, 1077 bp, synthetic construct
  source 1..1077: mol_type = other DNA
atggcactgcccgtgaccgctctgctgctgcctctcgccctgctgctgcatgctgccaggccccaagtcaagctcgaggagtctggtggtgggctcgtccaagctggcaggtccctgagactgagttgtgccgcctccgagcacactttcagctctcacgtgatgggctggttcaggcaggcacccggtaaggagcgggagtctgtggctgttatcgggtggcgggatatctctacctcctacgccgactccgtcaagggccggttcaccatctccagggacaacgcaaagaagaccctctacctccagatgaactcactcaagcccgaggacaccgctgtgtactactgcgcagccagacgcatcgatgccgcagacttcgactcctggggccaaggtacccaagtgacagtgtccagcatcgaggtgatgtacccacctccctacctcgacaacgagaagagcaacggcaccatcatccacgtgaaggggaagcacctgtgtccctctccccttttcccaggaccctccaagccattctgggtcctggtcgttgtcggaggcgtgctcgcttgctattccctgctcgtcaccgtggccttcatcatcttctgggtgcggtccaagagatcccggctgctgcactctgattacatgaacatgacacccaggaggccagggcctaccaggaagcactaccagccctacgctcctccacgcgacttcgcagcataccggtcccgcgtcaagttctcccggtccgcagatgctccagcctatcagcagggccagaaccagctgtacaacgaactcaatttggggaggcgcgaggagtatgatgtgctcgataagagaaggggccgggatcctgagatgggaggcaagcccagacggaagaaccctcaggagggcctgtacaatgagctgcagaaggacaagatggccgaggcctactccgagatcgggatgaagggtgaaagaaggaggggtaaggggcacgacgggctctaccaaggcctgagcaccgctaccaaggacacctacgatgcactgcatatgcaagccctgccaccacgg

SEQ ID NO: 315 - DNA, 1074 bp, synthetic construct
  source 1..1074: mol_type = other DNA
atggccctccctgtcacagccctcttgctgcccctcgcactccttctgcacgcagctcggcctgaggtccagctggtcgagagtggaggcggactggtccaggcaggtgggagtcttcggctttcctgcgcagcttccggacggaccttcaccatgggttggttccggcaggcacctgggaaggagagggagttcgttgccgctatcagcctctcaccaaccctggcctactatgcagagagcgtcaagggccgcttcaccatcagccgcgacaacgccaagaacaccgtcgtgctgcagatgaactccctcaagcccgaggataccgccctgtactactgcgctgccgatcggaagtccgtcatgtccattcggcccgactactggggacagggcacacaggtgaccgtcagcagcatcgaggtcatgtaccctccaccctacctggacaacgagaagagcaacgggaccatcatccacgtgaaggggaagcacctctgtccaagtcccctcttcccaggaccctccaagccattctgggtcctcgtggtggttggaggagtgctcgcctgctactctctgctggtgaccgtcgccttcatcatcttctgggtgcggtccaagcggtctcgcctcctccactccgactacatgaacatgacaccacgcagacctgggcccactaggaagcactatcagccctatgcaccaccccgggatttcgcagcctaccggtcacgggtgaagttcagcagatccgcagacgcaccagcctaccagcaggggcagaaccagctgtataacgagctgaacctcggtcgcagggaggagtacgatgtcctggataagagaaggggcagggatcccgagatgggtggcaagcccagacggaagaatcctcaggaggggctctacaacgagctgcagaaggacaagatggccgaggcttactcagagatcggcatgaaaggggagaggaggcgcggaaaaggccacgacggcctctaccagggactgtccaccgcaaccaaggatacctacgacgccctgcacatgcaagccctcccacctcgg

SEQ ID NO: 316 - DNA, 2232 bp, synthetic construct
  source 1..2232: mol_type = other DNA
atggctctccccgtgaccgctctcctccttcccttggccttgttgctgcacgccgcaaggcctcaggtgaagctggaggaatccggaggcggactcgtccaagcaggtcggtccctcaggctgtcttgcgctgccagcgagcacaccttctctagccacgtgatgggttggttcagacaggccccaggaaaggagcgggaatccgtggcagtgatcggctggcgggacatcagcacctcctacgccgactccgtcaagggccggttcaccatcagccgcgacaacgccaagaagaccctgtacctgcagatgaacagcctcaagcctgaggacaccgccgtgtactactgcgccgccagaaggatcgacgccgcagacttcgactcctggggtcagggaacccaggtgaccgtgtcctccacctctaccaccacaccagcacccagacctcctactcccgctcccaccatcgcttcccagcccctgtccctcagacccgaagcctgcagaccagcagctggcggtgcagtgcacaccaggggtcttgacttcgcctgtgacatctacatctgggctccactggctgggacttgcggcgttctgctgctgagcctggtgatcaccctgtactgcaagcggggccggaagaagctgctctacatcttcaagcagcctttcatgcggcccgttcagaccacccaggaggaagacgggtgcagttgccgcttccctgaggaggaggagggaggatgcgagctgcgggtcaagttctctcggtccgctgatgccccagcctaccagcagggccagaaccagctctataacgagctgaacctcggtaggcgggaggagtacgacgtcctggacaaaaggaggggacgggatcctgagatgggaggaaagccacggcggaagaaccctcaagaggggctgtacaacgaactccagaaggacaagatggctgaggcttattctgagatcggcatgaagggagagcgcagacgcggcaagggacacgatggcctgtatcagggactgagcaccgccaccaaggatacctacgacgccctccatatgcaggctctcccaccacggggatctggcgaaggcagaggatctctgctgacatgcggcgacgtggaagagaaccctggacctatggccctccctgtcacagccctcttgctgcccctcgcactccttctgcacgcagctcggcctgaggtccagctggtcgagagtggaggcggactggtccaggcaggtgggagtcttcggctttcctgcgcagcttccggacggaccttcaccatgggttggttccggcaggcacctgggaaggagagggagttcgttgccgctatcagcctctcaccaaccctggcctactatgcagagagcgtcaagggccgcttcaccatcagccgcgacaacgccaagaacaccgtcgtgctgcagatgaactccctcaagcccgaggataccgccctgtactactgcgctgccgatcggaagtccgtcatgtccattcggcccgactactggggacagggcacacaggtgaccgtcagcagcatcgaggtcatgtaccctccaccctacctggacaacgagaagagcaacgggaccatcatccacgtgaaggggaagcacctctgtccaagtcccctcttcccaggaccctccaagccattctgggtcctcgtggtggttggaggagtgctcgcctgctactctctgctggtgaccgtcgccttcatcatcttctgggtgcggtccaagcggtctcgcctcctccactccgactacatgaacatgacaccacgcagacctgggcccactaggaagcactatcagccctatgcaccaccccgggatttcgcagcctaccggtcacgggtgaagttcagcagatccgcagacgcaccagcctaccagcaggggcagaaccagctgtataacgagctgaacctcggtcgcagggaggagtacgatgtcctggataagagaaggggcagggatcccgagatgggtggcaagcccagacggaagaatcctcaggaggggctctacaacgagctgcagaaggacaagatggccgaggcttactcagagatcggcatgaaaggggagaggaggcgcggaaaaggccacgacggcctctaccagggactgtccaccgcaaccaaggatacctacgacgccctgcacatgcaagccctcccacctcgg

SEQ ID NO: 317 - DNA, 2232 bp, synthetic construct
  source 1..2232: mol_type = other DNA
atggcactgcccgtcacagctctgctgctgcccttggccctcttgttgcacgcagcacggccagaggtgcaacttgtcgagagcggtggtggccttgtgcaggccggaggcagtttgcgcctcagttgtgcagcttctggccggaccttcaccatgggctggttcagacaggcccctggtaaggaacgcgagttcgtggccgccatcagcctgtccccaaccctggcctactacgcagagagcgtgaagggtcggttcaccatctcccgcgacaacgcaaagaacaccgtggtgctccagatgaactccctgaagccagaagacaccgccctgtactactgcgcagccgaccggaagagcgtgatgtccatccgccctgactactggggccaaggcacacaggtcacagtgtccagcacctccactaccactccagctccacgccctccaacacccgcaccaaccatcgccagccagcctctgagtctgagacccgaagcatgccggccagctgctggaggtgccgtgcacaccagagggctggacttcgcctgcgacatctacatctgggctcctctggccggaacttgcggggtgctgctcctctcactggtcatcaccctgtactgcaagaggggcaggaagaagctcctgtacatcttcaagcagcccttcatgcggccagtccagacaacccaggaggaagacggatgcagctgtcgcttccccgaggaggaggaaggcggctgcgaattgcgggtcaagttcagcagatccgctgacgctcctgcctaccaacagggacagaaccagctctacaacgagctgaacctgggaaggcgggaggagtacgacgtcctggacaagagaagaggacgcgaccccgagatgggaggtaagcccagacgcaagaaccctcaagagggactgtataacgagctgcagaaggacaagatggccgaggcctacagcgagatcggcatgaagggtgaaagaagacggggaaaggggcacgacggtctgtatcaggggctctccaccgcaaccaaggatacctatgacgctctgcacatgcaggcactccctccacgcggatctggcgaaggcagaggatctctgctgacatgcggcgacgtggaagagaaccctggacctatggcactgcccgtgaccgctctgctgctgcctctcgccctgctgctgcatgctgccaggccccaagtcaagctcgaggagtctggtggtgggctcgtccaagctggcaggtccctgagactgagttgtgccgcctccgagcacactttcagctctcacgtgatgggctggttcaggcaggcacccggtaaggagcgggagtctgtggctgttatcgggtggcgggatatctctacctcctacgccgactccgtcaagggccggttcaccatctccagggacaacgcaaagaagaccctctacctccagatgaactcactcaagcccgaggacaccgctgtgtactactgcgcagccagacgcatcgatgccgcagacttcgactcctggggccaaggtacccaagtgacagtgtccagcatcgaggtgatgtacccacctccctacctcgacaacgagaagagcaacggcaccatcatccacgtgaaggggaagcacctgtgtccctctccccttttcccaggaccctccaagccattctgggtcctggtcgttgtcggaggcgtgctcgcttgctattccctgctcgtcaccgtggccttcatcatcttctgggtgcggtccaagagatcccggctgctgcactctgattacatgaacatgacacccaggaggccagggcctaccaggaagcactaccagccctacgctcctccacgcgacttcgcagcataccggtcccgcgtcaagttctcccggtccgcagatgctccagcctatcagcagggccagaaccagctgtacaacgaactcaatttggggaggcgcgaggagtatgatgtgctcgataagagaaggggccgggatcctgagatgggaggcaagcccagacggaagaaccctcaggagggcctgtacaatgagctgcagaaggacaagatggccgaggcctactccgagatcgggatgaagggtgaaagaaggaggggtaaggggcacgacgggctctaccaaggcctgagcaccgctaccaaggacacctacgatgcactgcatatgcaagccctgccaccacgg

SEQ ID NO: 318 - DNA, 1098 bp, synthetic construct
  source 1..1098: mol_type = other DNA
atggctctgcctgtgacagctctgctgctgcctctggctctgcttctgcatgccgctagacctgaggtgcagctggttgaatctggcggaggactggttcagcctggcggatctctgagactgagctgttctgccagcggcttcaccctggactactacatgatcggctggttcagacaggcccctggcaaagagagagagggcctgagcagcatctctcctgccgatggcagcacctactacgccgattctgtgaagggcagattcaccatcagccgggacagcagcaagaacaccgtgtacctgcagatgaacagcctgagagccgaggacaccgccgtgtactattgtgccgccggaaatgaggccacaatcagctggggatttggcccctgggagtacgattattggggccagggcaccatggtcaccgtgtcctctatcgaagtgatgtaccctcctccttacctggacaacgagaagtccaacggcaccatcatccacgtgaagggaaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgctgatgcccctgcctatcagcagggccagaaccagctgtacaacgagctgaacctggggagaagagaagagtacgacgtgctggacaagcggagaggcagagatcctgagatgggcggcaagcccagacggaagaatcctcaagagggactgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 319 - DNA, 1089 bp, synthetic construct
  source 1..1089: mol_type = other DNA
atggctctgcctgtgacagctctgctgctgcctctggctctgcttctgcatgccgctagacctgaggtgcagctggttgaatctggcggaggactggttcagcctggcggatctctgagactgtcttgtgccgccagcgagagcatcagcagcatccacatcatggcctggtacagacaggcccctggcaagcagagagaactggttgccggcatcagaaacgacggcagcacagtgtacgtggacagcgtgaagggcagattcaccatcagccgggacaacgccaagaacagcgtgtacctgcagatgaacagcctgagagccgaggacaccgccgtgtactactgcaatgccgatcaaggcttcggcagctacagcgagtgggagagaagatccagatggggccagggcaccaccgtgacagtgtctagcatcgaagtgatgtaccctccaccttacctggacaacgagaagtccaacggcaccatcatccacgtgaagggaaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgctgatgcccctgcctatcagcagggccagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctactccgagatcggaatgaagggcgagcgcagaagaggcaagggacacgatggactgtatcagggcctgagcaccgccaccaaggatacctatgatgccctgcacatgcaggccctgcctccaaga

SEQ ID NO: 320 - DNA, 1110 bp, synthetic construct
  source 1..1110: mol_type = other DNA
atggctctgcctgtgacagctctgctgctgcctctggctctgcttctgcatgccgctagacctgaggtgcagctggttgaatctggcggaggactggttcagcctggcggatctctgagactgagctgttctgccagcggcttcaccctggactactacatgatcggctggttcagacaggcccctggcaaagagagagagggcctgagcagcatctctcctgccgatggcagcacctactacgccgattctgtgaagggcagattcaccatcagccgggacagcagcaagaacaccgtgtacctgcagatgaacagcctgagagccgaggacaccgccgtgtactattgtgccgccggaaatgaggccacaatcagctggggatttggcccctgggagtacgattattggggccagggcaccatggtcaccgtgtcctctacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgctgacgcccctgcctatcagcagggccagaaccagctgtacaacgagctgaacctggggagaagagaagagtacgacgtgctggacaagcggagaggcagagatcctgagatgggcggcaagcccagacggaagaatcctcaagagggactgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 321 - DNA, 1482 bp, synthetic construct
  source 1..1482: mol_type = other DNA
atggctctgcctgtgacagctctgctgctgcctctggctctgcttctgcatgccgctagacctgaggtgcagctggttgaatctggcggaggactggttcagcctggcggatctctgagactgagctgttctgccagcggcttcaccctggactactacatgatcggctggttcagacaggcccctggcaaagagagagagggcctgagcagcatctctcctgccgatggcagcacctactacgccgattctgtgaagggcagattcaccatcagccgggacagcagcaagaacaccgtgtacctgcagatgaacagcctgagagccgaggacaccgccgtgtactattgtgccgccggaaatgaggccacaatcagctggggatttggcccctgggagtacgattattggggccagggcaccatggtcacagtgtctagtggcggcggaggcagtgaagtccagcttgttgaaagcggaggcggccttgtgcaacctggtggctctctcagactgtcctgtgccgccagcgagagcatcagcagcatccacatcatggcctggtataggcaggctccaggcaagcagagagagctggttgccggcatcagaaacgatggctctaccgtgtacgtggacagcgtgaaaggccggttcaccatctccagagacaacgccaagaattctgtgtatctccagatgaattccctgcgcgccgaagatacagccgtgtattactgcaacgccgaccaaggcttcggcagctacagcgaatgggagagaagatccagatggggacagggaaccaccgtgaccgtgtccagcatcgaagtgatgtaccctccaccttacctggacaacgagaagtccaacggcaccatcatccacgtgaagggaaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgctgatgcccctgcctatcagcagggccagaaccagctgtacaacgagctgaacctggggagaagagaagagtacgacgtgctggacaagcggagaggcagagatcctgagatgggcggcaagcccagacggaagaatcctcaagagggactgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 322 - DNA, 1494 bp, synthetic construct
  source 1..1494: mol_type = other DNA
atggctctgcctgtgacagctctgctgctgcctctggctctgcttctgcatgccgctagacctgaggtgcagctggttgaatctggcggaggactggttcagcctggcggatctctgagactgagctgttctgccagcggcttcaccctggactactacatgatcggctggttcagacaggcccctggcaaagagagagagggcctgagcagcatctctcctgccgatggcagcacctactacgccgattctgtgaagggcagattcaccatcagccgggacagcagcaagaacaccgtgtacctgcagatgaacagcctgagagccgaggacaccgccgtgtactattgtgccgccggaaatgaggccacaatcagctggggatttggcccctgggagtacgattattggggccagggcaccatggtcacagtgtctagtggcggcggaggcagtgaagtccagcttgttgaaagcggaggcggccttgtgcaacctggtggctctctcagactgtcctgtgccgccagcgagagcatcagcagcatccacatcatggcctggtataggcaggctccaggcaagcagagagagctggttgccggcatcagaaacgatggctctaccgtgtacgtggacagcgtgaaaggccggttcaccatctccagagacaacgccaagaattctgtgtatctccagatgaattccctgcgcgccgaagatacagccgtgtattactgcaacgccgaccaaggcttcggcagctacagcgaatgggagagaagatccagatggggacagggaaccaccgtgaccgtgtccagcacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgctgacgcccctgcctatcagcagggccagaaccagctgtacaacgagctgaacctggggagaagagaagagtacgacgtgctggataagcggagaggcagagatcctgagatgggcggcaagcccagacggaagaatcctcaagagggactgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 323 - DNA, 1101 bp, synthetic construct
  source 1..1101: mol_type = other DNA
atggctctgcctgtgacagctctgctgctgcctctggctctgcttctgcatgccgctagacctgaggtgcagctggttgaatctggcggaggactggttcagcctggcggatctctgagactgtcttgtgccgccagcgagagcatcagcagcatccacatcatggcctggtacagacaggcccctggcaagcagagagaactggttgccggcatcagaaacgacggcagcacagtgtacgtggacagcgtgaagggcagattcaccatcagccgggacaacgccaagaacagcgtgtacctgcagatgaacagcctgagagccgaggacaccgccgtgtactactgcaatgccgatcaaggcttcggcagctacagcgagtgggagagaagatccagatggggccagggcaccaccgtgacagtgtctagcacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgctgacgcccctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctactccgagatcggaatgaagggcgagcgcagaagaggcaagggacacgatggactgtatcagggcctgagcaccgccaccaaggatacctatgatgccctgcacatgcaggccctgcctccaaga

SEQ ID NO: 324 - DNA, 1107 bp, synthetic construct
  source 1..1107: mol_type = other DNA
atggctctgcctgtgacagctctgctgctgcctctggctctgcttctgcatgccgctagacctgaggttcagctgcaagcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctgcagatgaacagcctgagagccgaggataccgccgtgtacttctgtgccagcagaggaatcgccgacggcagcgattttggctcttatggccagggcacccaagtgaccgttagctctcctgccaagcctaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaatctggggcgcagagaagagtacgacgtgctggacaagagaagaggcagggaccctgagatgggcggaaagccccagagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 325 - DNA, 1095 bp, synthetic construct
  source 1..1095: mol_type = other DNA
atggctctgcctgtgacagctctgctgctgcctctggctctgcttctgcatgccgctagacctgaggttcagctgcaagcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctgcagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaagcctgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaatctggggcgcagagaagagtacgacgtgctggataagcggagaggcagagatcctgagatgggcggaaagccccagcggagaaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 326 - DNA, 1083 bp, synthetic construct
  source 1..1083: mol_type = other DNA
atggctctgcctgtgacagctctgctgctgcctctggctctgcttctgcatgccgctagacctgaggttcagctgcaagcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctgcagatgaacagcctgagagccgaggataccgccgtgtacttctgtgccagcagaggaatcgccgacggcagcgattttggctcttatggccagggcacccaagtgaccgtgtccagcatcgaagtgatgtaccctccaccttacctggacaacgagaagtccaacggcaccatcatccacgtgaagggaaagcacctgtgtccttctccactgttccccggacctagcaagcctttctgggtgctcgttgttgttggcggcgtgctggcctgttactctctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtccagagtgaagttcagcagatccgccgatgctcccgcctatcagcagggacagaaccagctgtacaacgagctgaatctggggcgcagagaagagtacgacgtgctggataagcggagaggcagagatcctgagatgggcggaaagccccagcggagaaagaatcctcaagagggcctgtataatgagctgcagaaagacaagatggccgaggcctacagcgagatcggaatgaagggcgaacgcagaagaggcaagggccacgatggactgtatcagggcctgagcaccgccaccaaggatacctatgatgccctgcacatgcaggccctgcctccaaga

SEQ ID NO: 327 - protein, 475 aa, synthetic construct
MGVPTQLLGLLLLWITDAICDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 328 - protein, 365 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 329 - protein, 364 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 330 - protein, 359 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 331 - protein, 358 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 332 - protein, 744 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 333 - protein, 744 aa, synthetic construct
MALPVTALLLPLALLLHAARPEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 334 - protein, 366 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 335 - protein, 363 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 336 - protein, 370 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 337 - protein, 494 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 338 - protein, 498 aa, synthetic construct
MALPVTALLLPLALLLHAARPEVQLVESGGGLVQPGGSLRLSCSASGFTLDYYMIGWFRQAPGKEREGLSSISPADGSTYYADSVKGRFTISRDSSKNTVYLQMNSLRAEDTAVYYCAAGNEATISWGFGPWEYDYWGQGTMVTVSSGGGGSEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 339 - protein, 367 aa, synthetic construct
MALPVTALLLPLALLLHAARPEVQLVESGGGLVQPGGSLRLSCAASESISSIHIMAWYRQAPGKQRELVAGIRNDGSTVYVDSVKGRFTISRDNAKNSVYLQMNSLRAEDTAVYYCNADQGFGSYSEWERRSRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 340 - protein, 369 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 341 - protein, 365 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 342 - protein, 361 aa, synthetic construct
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 343 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 443 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 346 - protein, 209 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 347 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 451 aa, synthetic construct
QVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 213 aa, synthetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 352 - protein, 211 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 353 - protein, 448 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 355 - protein, 451 aa, synthetic construct
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 356 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 447 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 210 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 359 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 361 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 362 - protein, 122 aa, synthetic construct
EVQLQQSGAELKKPGASVKVSCKASGYTFTSYNMHWVKQTPGQGLEWIGAIYPGNGDTSYNQKFKGKTTLTADKSSSTAYMELSSLRSEDTAVYYCARSNYYGSSYWFFDVWGTGTTVTVSS

SEQ ID NO: 363 - protein, 106 aa, synthetic construct
DIVLTQSPAIITASPGEKVTMTCRASSSVNYMDWYQKKPGSSPKPWIYATSNLASGVPSRFSGSGSGTTYSMTISSLEAEDAATYYCQQWSFNPPTFGGGTKLEIK

SEQ ID NO: 364 - protein, 245 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTG

SEQ ID NO: 365 - protein, 250 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTGLEIGRTG

SEQ ID NO: 366 - protein, 249 aa, synthetic construct
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 367 - protein, 252 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKY

SEQ ID NO: 368 - protein, 252 aa, synthetic construct
DIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKLELKGSTSGGGSGGGSGGGGSSQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTTLTVSSLDPKSS

SEQ ID NO: 369 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 370 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 371 - protein, 119 aa, synthetic construct
EVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSS

SEQ ID NO: 372 - protein, 110 aa, synthetic construct
EIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIK

SEQ ID NO: 373 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 374 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 375 - protein, 120 aa, synthetic construct
QVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSS

SEQ ID NO: 376 - protein, 106 aa, synthetic construct
ENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIK

SEQ ID NO: 377 - protein, 120 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 378 - protein, 106 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIK

SEQ ID NO: 379 - protein, 121 aa, synthetic construct
EVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSS

SEQ ID NO: 380 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 381 - protein, 120 aa, synthetic construct
QVQLVQSGAEVKKPGSSVKVSCKASGHTISSYAYSWVRQAPGQGLEWMGDIIPAYGSPNYAQKFQGRVTITADESTSTAYMELSSLRSEDTAVYYCAREDFGKNYAMDVWGQGTLVTVSS

SEQ ID NO: 382 - protein, 108 aa, synthetic construct
EIVLTQSPGTLSLSPGERATLSCRASQHVSSHYLAWYQQKPGQAPRLLIYGASSRATGIPDRFSGSGSGTDFTLTISRLEPEDFAVYYCQHYGQSQFTFGQGTKVEIK

SEQ ID NO: 383 - protein, 121 aa, synthetic construct
QVQLVQSGAEVKKPGASVKVSCKASGYDFTDYIMHWVRQAPGQCLEWMGYINPYNDGSKYTDKFQERVTMTSDTSISTAYMELSRLRSDDTAVYYCARGTYYYGPELFDYWGQGTTVTVSS

SEQ ID NO: 384 - protein, 112 aa, synthetic construct
DIVMTQTPLSLSVTPGQPASISCKSSQSLETTTGTTYLNWYLQKPGQSPQLLIYRASKRFSGVPDRFSGSGSGTDFTLKISRVEAEDVGVYYCLQLLEDPYTFGCGTKLEIK

SEQ ID NO: 385 - protein, 120 aa, synthetic construct
QVQLQESGPGLVKPSQTLSLTCTVSGGSISTSTMGVGWIRQHPGKGLEWIGFIWWDDDKRYNPNLKSRVTMSVDTSKNQFSLKLSSVTAADTAVYYCARMELWSYYFDYWGQGTLVTVSS

SEQ ID NO: 386 - protein, 106 aa, synthetic construct
EIVLTQSPATLSLSPGERATLSCSASSSVGYMHWYQQKPGQAPRLLIYDTSKLASGIPARFSGSGSGTDFTLTISSLEPEDFAVYYCFQGSVYPFTFGQGTKLEIK

SEQ ID NO: 387 - protein, 121 aa, synthetic construct
QVQLVQSGAEVKKPGSSVKVSCKASGYTFTDYVIHWVRQAPGQGLEWMGYFNPYNDGTEYNEKFKARVTITADKSTSTAYMELSSLRSEDTAVYYCARGPYYYGSSPFDYWGQGTTVTVSS

SEQ ID NO: 388 - protein, 112 aa, synthetic construct
DIVMTQTPLSLPVTPGEPASISCRSSQSLENSNHNTYINWYLQKPGQSPQLLIYRVSKRFSGVPDRFSGSGSGTDFTLKISRVEAEDVGVYYCHQVTHVPYTFGQGTKLEIK

SEQ ID NO: 389 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 390 - protein, 247 aa, synthetic construct
EIVLTQSPATLSLSPGERATLSCSASSSISSNYLHWYQQKPGQAPRFLIYRTSNLASGIPARFSGSGSGTDFTLTISSLEPEDFAVYYCQQASSIPRMFTFGQGTKLEIKGSTSGSGKPGSGEGSTKGEVQLQQSGAEVKKPGASVKVSCKASGYTFTNYWMHWVRQRPGQGLEWMGEIDPSDNYANYNQEFQGRVTITVDKSASTAYMELSSLRSEDTAVYYCARHDGYFGALDYWGQGTTVTVSS

SEQ ID NO: 391 - protein, 248 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 392 - protein, 241 aa, synthetic construct
QVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSGGGGSGGGGSGGGGSENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIK

SEQ ID NO: 393 - protein, 241 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKGGGGSGGGGSGGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 394 - protein, 243 aa, synthetic construct
EVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 395 - protein, 243 aa, synthetic construct
QVQLVQSGAEVKKPGSSVKVSCKASGHTISSYAYSWVRQAPGQGLEWMGDIIPAYGSPNYAQKFQGRVTITADESTSTAYMELSSLRSEDTAVYYCAREDFGKNYAMDVWGQGTLVTVSSGGGGSGGGGSGGGGSEIVLTQSPGTLSLSPGERATLSCRASQHVSSHYLAWYQQKPGQAPRLLIYGASSRATGIPDRFSGSGSGTDFTLTISRLEPEDFAVYYCQHYGQSQFTFGQGTKVEIK

SEQ ID NO: 396 - protein, 243 aa, synthetic construct
EIVLTQSPGTLSLSPGERATLSCRASQHVSSHYLAWYQQKPGQAPRLLIYGASSRATGIPDRFSGSGSGTDFTLTISRLEPEDFAVYYCQHYGQSQFTFGQGTKVEIKGGGGSGGGGSGGGGSQVQLVQSGAEVKKPGSSVKVSCKASGHTISSYAYSWVRQAPGQGLEWMGDIIPAYGSPNYAQKFQGRVTITADESTSTAYMELSSLRSEDTAVYYCAREDFGKNYAMDVWGQGTLVTVSS

SEQ ID NO: 397 - protein, 248 aa, synthetic construct
DIVMTQTPLSLSVTPGQPASISCKSSQSLETTTGTTYLNWYLQKPGQSPQLLIYRASKRFSGVPDRFSGSGSGTDFTLKISRVEAEDVGVYYCLQLLEDPYTFGCGTKLEIKGGGGSGGGGSGGGGSQVQLVQSGAEVKKPGASVKVSCKASGYDFTDYIMHWVRQAPGQCLEWMGYINPYNDGSKYTDKFQERVTMTSDTSISTAYMELSRLRSDDTAVYYCARGTYYYGPELFDYWGQGTTVTVSS

SEQ ID NO: 398 - protein, 241 aa, synthetic construct
EIVLTQSPATLSLSPGERATLSCSASSSVGYMHWYQQKPGQAPRLLIYDTSKLASGIPARFSGSGSGTDFTLTISSLEPEDFAVYYCFQGSVYPFTFGQGTKLEIKGGGGSGGGGSGGGGSQVQLQESGPGLVKPSQTLSLTCTVSGGSISTSTMGVGWIRQHPGKGLEWIGFIWWDDDKRYNPNLKSRVTMSVDTSKNQFSLKLSSVTAADTAVYYCARMELWSYYFDYWGQGTLVTVSS

SEQ ID NO: 399 - protein, 258 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 400 - protein, 249 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKGGGGSGGGSGGGSGGGGSGGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 401 - protein, 41 aa, synthetic construct
KRGRKKLLYIFKQPFMRPVQTTQEEDGCSCRFPEEEEGGCE

SEQ ID NO: 402 - protein, 241 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 403 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 404 - DNA, 23 bp, synthetic construct
  source 1..23: mol_type = other DNA
atgggagtgaaggtgctgttcgc

SEQ ID NO: 405 - DNA, 37 bp, synthetic construct
  source 1..37: mol_type = other DNA
tttttgttggtttgtgtttgttttgtttggtgtttgc

SEQ ID NO: 406 - protein, 5 aa, synthetic construct
SYVMH

SEQ ID NO: 407 - protein, 6 aa, synthetic construct
NPYNDG

SEQ ID NO: 408 - protein, 12 aa, synthetic construct
GTYYYGTRVFDY

SEQ ID NO: 409 - protein, 16 aa, synthetic construct
RSSKSLQNVNGNTYLY

SEQ ID NO: 410 - protein, 7 aa, synthetic construct
RMSNLNS

SEQ ID NO: 411 - protein, 9 aa, synthetic construct
MQHLEYPIT

SEQ ID NO: 412 - protein, 7 aa, synthetic construct
TSTMGVG

SEQ ID NO: 413 - protein, 16 aa, synthetic construct
FIWWDDDKRYNPNLKS

SEQ ID NO: 414 - protein, 10 aa, synthetic construct
MELWSYYFDY

SEQ ID NO: 415 - protein, 10 aa, synthetic construct
SASSSVGYMH

SEQ ID NO: 416 - protein, 7 aa, synthetic construct
DTSKLAS

SEQ ID NO: 417 - protein, 9 aa, synthetic construct
FQGSVYPFT

SEQ ID NO: 418 - protein, 7 aa, synthetic construct
TSGMGVG

SEQ ID NO: 419 - protein, 5 aa, synthetic construct
WWDDD

SEQ ID NO: 420 - protein, 10 aa, synthetic construct
MELWSYYFDY

SEQ ID NO: 421 - protein, 6 aa, synthetic construct
SVSYMH

SEQ ID NO: 422 - protein, 7 aa, synthetic construct
DASNRAS

SEQ ID NO: 423 - protein, 10 aa, synthetic construct
FQGSVYPFTF

SEQ ID NO: 424 - protein, 13 aa, synthetic construct
KASGHTISSYAYS

SEQ ID NO: 425 - protein, 10 aa, synthetic construct
DIIPAYGSPN

SEQ ID NO: 426 - protein, 13 aa, synthetic construct
AREDFGKNYAMDV

SEQ ID NO: 427 - protein, 12 aa, synthetic construct
RASQHVSSHYLA

SEQ ID NO: 428 - protein, 8 aa, synthetic construct
YGASSRAT

SEQ ID NO: 429 - protein, 9 aa, synthetic construct
QHYGQSQFT

SEQ ID NO: 430 - protein, 5 aa, synthetic construct
DYIMH

SEQ ID NO: 431 - protein, 17 aa, synthetic construct
YINPYNDGSKYTDKFQE

SEQ ID NO: 432 - protein, 12 aa, synthetic construct
GTYYYGPELFDY

SEQ ID NO: 433 - protein, 16 aa, synthetic construct
KSSQSLETTTGTTYLN

SEQ ID NO: 434 - protein, 7 aa, synthetic construct
RASKRFS

SEQ ID NO: 435 - protein, 9 aa, synthetic construct
LQLLEDPYT